(19) United States
(12) Patent Application Publication
(10) Pub. No.: US 2026/0049053 A1
(43) Pub. Date: Feb. 19, 2026

(54) ONIUM SALT, PHOTOACID GENERATOR, CHEMICALLY AMPLIFIED RESIST COMPOSITION, AND PATTERNING PROCESS

(71) Applicant: SHIN-ETSU CHEMICAL CO., LTD., Tokyo (JP)

(72) Inventor: Masahiro Fukushima, Joetsu-shi (JP)

(73) Assignee: SHIN-ETSU CHEMICAL CO., LTD., Tokyo (JP)

(21) Appl. No.: 19/293,577

(22) Filed: Aug. 7, 2025

(30) Foreign Application Priority Data
Aug. 13, 2024 (JP) 2024-134761

(51) Int. Cl. C07C 381/12; G03F 7/004

(52) U.S. Cl. CPC C07C 381/12; G03F 7/0045

(57) ABSTRACT

The present invention is an onium salt, wherein the onium salt includes an anion represented by the following general formula (1A) and a cation represented by the following general formula (1B). This provides: an onium salt used as a photoacid generator contained in a chemically amplified resist composition having excellent solvent solubility, high sensitivity, high contrast, and excellent lithography performance in photolithography using high-energy beams; a photoacid generator consisting of the onium salt; a chemically amplified resist composition containing the photoacid generator; and a patterning process using the chemically amplified resist composition.

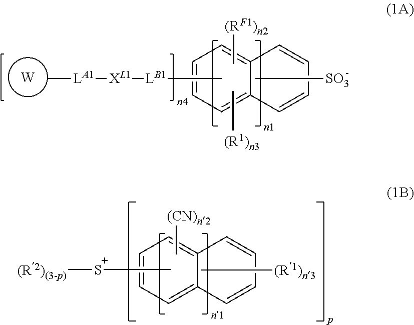

### TECHNICAL FIELD

The present invention relates to: an onium salt; a photoacid generator; a chemically amplified resist composition; and a patterning process.

### BACKGROUND ART

In recent years, along with the increasing integration and speed of LSIs, there has been a demand for finer pattern rules, and far ultraviolet and extreme ultraviolet (EUV) lithographies are seen as promising next-generation fine processing technologies.

Photolithography using ArF excimer laser light (ArF lithography) has begun to be used partially in the production of 130 nm node devices, and has become the main lithography technology for 90 nm node devices. Lithography using a 157 nm wavelength F₂ laser was initially seen as a promising lithography technology for the next 45 nm node, but various problems were noted that caused delays in development, so ArF immersion lithography, which can achieve high resolution by inserting a liquid with a higher refractive index than air, such as water, ethylene glycol, or glycerin, between the projection lens and the wafer, has been put to practical use, allowing the numerical aperture (NA) of the projection lens to be designed to be 1.0 or more. This immersion lithography requires resist compositions that are not easily dissolved in water.

In ArF lithography, there is a demand for highly sensitive resist compositions that can provide sufficient resolution with a small amount of exposure in order to prevent deterioration of precise and expensive optical materials. The most common way to achieve this is to select components that are highly transparent at a wavelength of 193 nm. For example, with regard to the base polymer, polyacrylic acid and its derivatives, norbornene-maleic anhydride alternating polymers, polynorbornene, ring-opening metathesis polymers, and ring-opening metathesis polymer hydrogen additives have been proposed, and some degree of success has been achieved in terms of increasing the transparency of the resin on its own.

In recent years, negative tone resists developed with organic solvents have been attracting attention, along with positive tone resists developed with alkaline aqueous solutions. In order to resolve extremely fine hole patterns that cannot be achieved with positive tone exposure, to resolve with negative tone exposure, a positive resist composition with high resolution is used, and a negative pattern is formed by developing with an organic solvent. Furthermore, research is underway to obtain twice the resolution by combining two developments, one with an alkaline aqueous solution and one with an organic solvent. For an ArF resist composition for negative tone development using an organic solvent, a conventional positive ArF resist composition can be used, and a patterning process using this composition is described in Patent Documents 1 to 3.

In order to accommodate the rapid miniaturization in recent years, development of resist compositions is progressing day by day along with process technologies. Various studies have been conducted on photoacid generators, and sulfonium salts consisting of triphenylsulfonium cations and perfluoroalkane sulfonate anions are commonly used. However, the acids generated, namely, perfluoroalkane sulfonic acids, and in particular perfluorooctane sulfonic acid (PFOS), are difficult to decompose, and there are concerns about bioconcentration and toxicity, making their use in resist compositions difficult; moreover, currently, photoacid generators that generate perfluorobutane sulfonic acid are used. However, when this compound is used in a resist composition, the acid generated diffuses significantly, making it difficult to achieve high resolution. In response to this problem, various partially fluorinated alkane sulfonic acids and their salts have been developed: for example, Patent Document 1 describes, as prior art, photoacid generators that generate α,α-difluoroalkane sulfonic acid upon exposure to light, specifically, photoacid generators that generate di(4-tert-butylphenyl)iodonium 1,1-difluoro-2-(1-naphthyl)ethanesulfonate and α,α,β,β-tetrafluoroalkane sulfonic acid. However, although the fluorine substitution rate of these compounds has been reduced, they do not have decomposable substituents such as ester structures, making them insufficient from the standpoint of environmental safety based on easy decomposition; furthermore, there are limitations on the molecular design for changing the size of the alkane sulfonic acids, and the starting materials containing fluorine atoms are expensive, among other problems.

Furthermore, as circuit line widths shrink, the impact of contrast degradation due to acid diffusion in resist compositions has become more severe. This is because the pattern dimensions approach the diffusion length of the acid, which causes the dimensional deviation on the wafer relative to the mask dimensional deviation (mask error factor (MEF)) to increase, resulting in a decrease in mask fidelity and a deterioration of pattern rectangularity. Therefore, in order to fully benefit from the shorter wavelength and higher NA of light sources, it is necessary to increase the dissolution contrast or suppress acid diffusion more than with conventional materials. One solution is to lower the bake temperature, which reduces acid diffusion and thus improves the MEF, but this inevitably results in a decrease in sensitivity.

Introducing bulky substituents or polar groups into photoacid generators is effective for suppressing acid diffusion. Patent Document 4 describes a photoacid generator having 2-acyloxy-1,1,3,3,3-pentafluoropropane-1-sulfonic acid, which has excellent solubility and stability in solvents and enables a wide range of molecular design possibilities; in particular, photoacid generators having 2-(1-adamantyloxy)-1,1,3,3,3-pentafluoropropane-1-sulfonic acid, which has a bulky substituent introduced therein, have low acid diffusion. Furthermore, Patent Documents 5 to 7 describe photoacid generators in which condensed ring lactones, sultones, or thiolactones are introduced as polar groups. Although it has been confirmed that the introduction of polar groups has an acid diffusion suppressing effect, improving performance to a certain extent, it is still insufficient to achieve a high level of control over acid diffusion, and the lithography performance is not satisfactory in view of the MEF, pattern shape, and sensitivity, etc. as a whole.

Introducing a polar group into the anion of a photoacid generator is effective for suppressing acid diffusion, but is disadvantageous in terms of solvent solubility. In Patent Documents 8 and 9, attempts have been made to improve solvent solubility by introducing an alicyclic group into the cation portion of the photoacid generator, specifically by introducing a cyclohexane ring or an adamantane ring. Although the introduction of such alicyclic groups improves solubility, a certain number of carbon atoms is necessary to ensure solubility, and as a result, the molecular structure of the photoacid generator becomes bulky, which deteriorates lithography performance such as line width roughness (LWR) and dimensional uniformity (CDU) when forming fine patterns.

Furthermore, Patent Document 10 describes a photoacid generator that generates a fluoroalkane sulfonic acid having an anion that is an aromatic condensed ring derived from anthracene. Although this has been shown to improve lithography performance to a certain extent, the alkane sulfonic acid structure lacks rigidity and has been subject to regulation in recent years as an organic fluorine compound subject to PFAS, raising concerns about its impact on the environment and human body.

Since iodine atoms have a very high absorption rate for EUV rays with a wavelength of 13.5 nm, it has been confirmed that secondary electrons are generated from iodine atoms during exposure, making this effect attractive for EUV lithography. Patent Document 11 describes a photoacid generator in which an iodine atom has been introduced into the anion, and Patent Document 12 describes a polymerizable group-containing photoacid generator in which an iodine atom has been introduced into the anion. Patent Document 13 describes a photoacid generator in which iodine atoms are introduced into both the cation and anion. Although this has been confirmed to improve lithography performance to a certain extent, iodine atoms are not highly soluble in organic solvents, and there is concern that they may precipitate in the solvent.

Patent Document 14 describes a photoacid generator in which a plurality of fluorine atoms are introduced into the cation. Although the introduction of a plurality of fluorine atoms improves the solvent solubility of the photoacid generator, this is not sufficient from the perspective of EUV absorption, and there is still room for improvement.

Patent Documents 15 to 19 describe photoacid generators and quenchers (acid diffusion control agents) whose cations contain iodine atoms and fluorine atoms. Although these developments have yielded confirmed improvements in the performance of resist materials, they are still not satisfactory in terms of acid diffusion control, and there is a demand for the development of resist materials that are more useful for forming finer patterns.

### CITATION LIST

### Patent Literature

Patent Document 1: JP2008-281974A
Patent Document 2: JP2008-281975A
Patent Document 3: JP4554665B2
Patent Document 4: JP2007-145797A
Patent Document 5: JP5061484B2
Patent Document 6: JP2016-147879A
Patent Document 7: JP2015-063472A
Patent Document 8: JP5573098B2
Patent Document 9: JP6461919B2
Patent Document 10: JP7109178B2
Patent Document 11: JP6720926B2
Patent Document 12: JP6973274B2
Patent Document 13: JP7041204B2
Patent Document 14: JP7389562B2
Patent Document 15: JP2021-123579A
Patent Document 16: JP2021-123580A
Patent Document 17: JP2022-123839A
Patent Document 18: JP2023-088869A
Patent Document 19: JP2023-088870A

### SUMMARY OF INVENTION

### Technical Problem

In response to recent demands for high resolution resist patterns, resist compositions that use conventional onium salt-type photoacid generators are unable to adequately suppress acid diffusion, which can result in degradation of lithography performance such as contrast, LWR, CDU, MEF, exposure latitude (EL), and depth of focus (DOF).

The present invention has been made in view of the above-described circumstances, and an object of the present invention is to provide: an onium salt used as a photoacid generator contained in a chemically amplified resist composition having excellent solvent solubility, high sensitivity, high contrast, and excellent lithography performance in photolithography using high-energy beams; a photoacid generator consisting of the onium salt; a chemically amplified resist composition containing the photoacid generator; and a patterning process using the chemically amplified resist composition.

### Solution to Problem

To achieve the object, the present invention provides an onium salt, wherein the onium salt comprises an anion represented by the following general formula (1A) and a cation represented by the following general formula (1B),

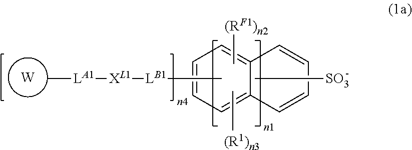

wherein “n1” represents 0 or 1; “n2” represents an integer from 0 to 4; “n3” represents an integer from 0 to 4; however, when “n1” represents 0, 0≤n2+n3≤4, and when “n1” represents 1, 0≤n2+n3≤6; “n4” represents 0 or 1;
W represents a hydrocarbyl group having 6 to 60 carbon atoms and containing at least one aromatic ring, and the hydrocarbyl group may contain a heteroatom;
R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” represents 2, 3, or 4, each R¹ may be the same as or different from each other, and a plurality of R¹ may be bonded to each other to form a ring together with the carbon atoms to which they are bonded;
L^{A1} and L^{B1} each independently represent a single bond, an ether bond, an ester bond, a sulfonate ester bond, an amide bond, a sulfonamide bond, a carbonate bond, or a carbamate bond; and
X^{L1} represents a single bond or a hydrocarbylene group having 1 to 40 carbon atoms and optionally containing a heteroatom;

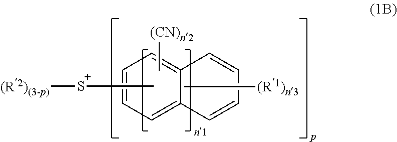

wherein “p” represents 1, 2, or 3; “n′1” represents 0 or 1; “n′2” represents 1 or 2; “n′3” represents an integer from 0 to 6; however, when “n′1” is 0, 1≤n′2+n′3≤5, and when “n′1” is 1, 1≤n′2+n′3≤7;
R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n′3” is 2 to 6, each R′¹ may be the same as or different from each other, and two R′¹ may be bonded to each other to form a ring together with the carbon atoms to which they are bonded;
R′² represents a halogen atom or a hydrocarbyl group having 1 to 30 carbon atoms and optionally containing a heteroatom; when “p” is 1, two R′² may be the same as or different from each other; and also, two of the three substituents bonded to S⁺ may be bonded to each other to form a ring with the sulfur atom to which they are bonded.

Such an onium salt is used, a chemically amplified resist composition containing it as a photoacid generator will have excellent solvent solubility, high sensitivity, high contrast, and excellent lithography performance in photolithography using high-energy beams.

Furthermore, in the present invention, the W is preferably represented by the following general formula (W-1) or (W-2),

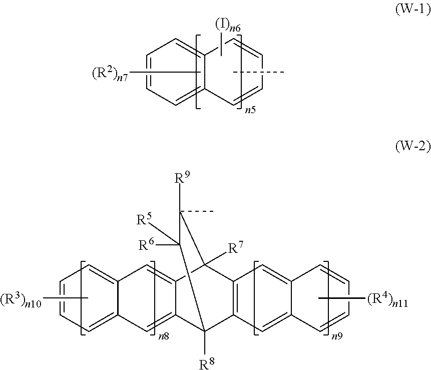

wherein “n5” represents 0 or 1; “n6” represents an integer from 0 to 4; “n7” represents an integer from 1 to 4; however, when “n5” represents 0, 1≤n6+n7≤5, and when “n5” represents 1, 1≤n6+n7≤7; “n8” represents 0 or 1; “n9” represents 0 or 1; “n10” represents an integer from 0 to 4; “n11” represents an integer from 0 to 4;
R² each independently represents a hydrogen atom, a halogen atom other than an iodine atom, or a hydrocarbyl group having 1 to 20 carbon atoms and optionally containing a heteroatom;
R³ and R⁴ each independently represent a hydrogen atom, a halogen atom, or a hydrocarbyl group having 1 to 20 carbon atoms and optionally containing a heteroatom;
R⁵ to R⁹ each independently represent a hydrogen atom, a halogen atom, or a hydrocarbyl group having 1 to 20 carbon atoms and optionally containing a heteroatom; and
a broken line represents an attachment point to L^{A1}.

The anion contained in the onium salt of the present invention preferably has such a group.

Furthermore, in the present invention, the anion is preferably represented by the following general formula (1A-1),

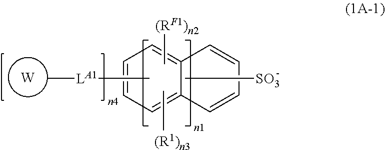

wherein “n1” to “n4”, W, R^{F1}, R¹, and L^{A1} are as defined above.

The onium salt of the present invention preferably has such an anion.

Furthermore, in the present invention, the cation is preferably represented by the following general formula (1B-1),

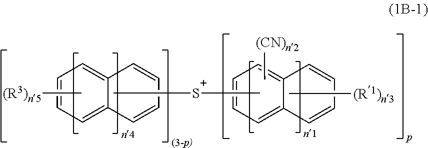

wherein “p”, “n′1” to “n′3”, and R′¹ are as defined above;
“n′4” represents 0 or 1; “n′5” represents an integer from 0 to 5;
R′³ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and when “n′5” represents 2 to 5, each R′³ may be the same as or different from each other, and two R′³ may be bonded to each other to form a ring together with the carbon atoms to which they are bonded.

The onium salt of the present invention preferably has such a cation.

The present invention also provides a photoacid generator, contains the onium salt described above.

Such a photoacid generator can be used as a photoacid generator contained in a chemically amplified resist composition that has excellent solvent solubility, high sensitivity, high contrast, and excellent lithography performance in photolithography using high-energy beams.

The present invention also provides a chemically amplified resist composition, contains the photoacid generator described above.

Such a chemically amplified resist composition can be used as a chemically amplified resist composition that has excellent solvent solubility, high sensitivity, high contrast, and excellent lithography performance in photolithography using high-energy beams.

Furthermore, in the present invention, it is preferable that the chemically amplified resist composition further contains a base polymer containing a repeating unit represented by either one or both of the following general formulae (a1) and (a2),

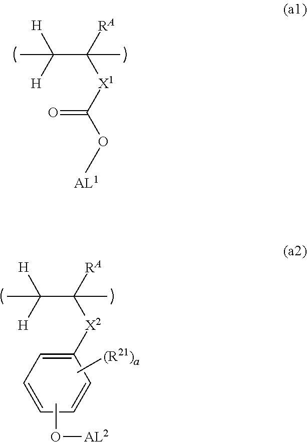

wherein R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
* represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a” represents an integer from 0 to 4.

The chemically amplified resist composition of the present invention preferably contains such a base polymer.

In this case, it is preferable that the base polymer further contains a repeating unit represented by the following general formula (a3),

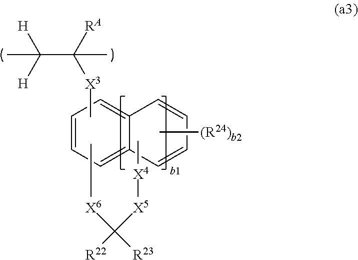

wherein “b1” represents 0 or 1; “b2” represents an integer from 0 to 3 when “b1” is 0, and an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; * represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; however, X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; furthermore, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, a plurality of R²⁴ may be bonded to each other to form a ring together with the carbon atoms of the aromatic ring to which they are bonded.

In the present invention, it is preferable that the base polymer has such a repeating unit.

It is also preferable that the base polymer further contains a repeating unit represented by either one or both of the following general formulae (b1) and (b2),

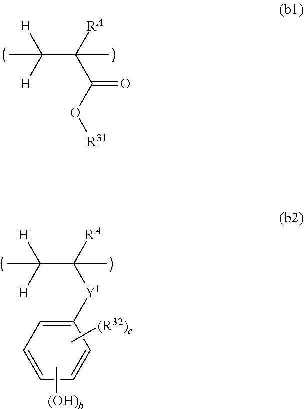

wherein R^{A} each independently represents a hydrogen atom, a fluorine atom, a methyl group, or a trifluoromethyl group;
Y¹ represents a single bond or *—C(═O)—O—; * represents an attachment point to a main chain carbon atom;
R³¹ represents a hydrogen atom, or a group having 1 to 20 carbon atoms and containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic acid anhydride (—C(═O)—O—C(═O)—);
R³² represents a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “c” is 2 or more, each R³² may be the same as or different from each other;
“b” represents an integer from 1 to 4; and “c” represents an integer from 0 to 4; however, 1≤b+c≤5.

In the present invention, it is preferable that the base polymer has such a repeating unit.

It is also preferable that the base polymer further contains at least one repeating unit selected from the group consisting of a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5),

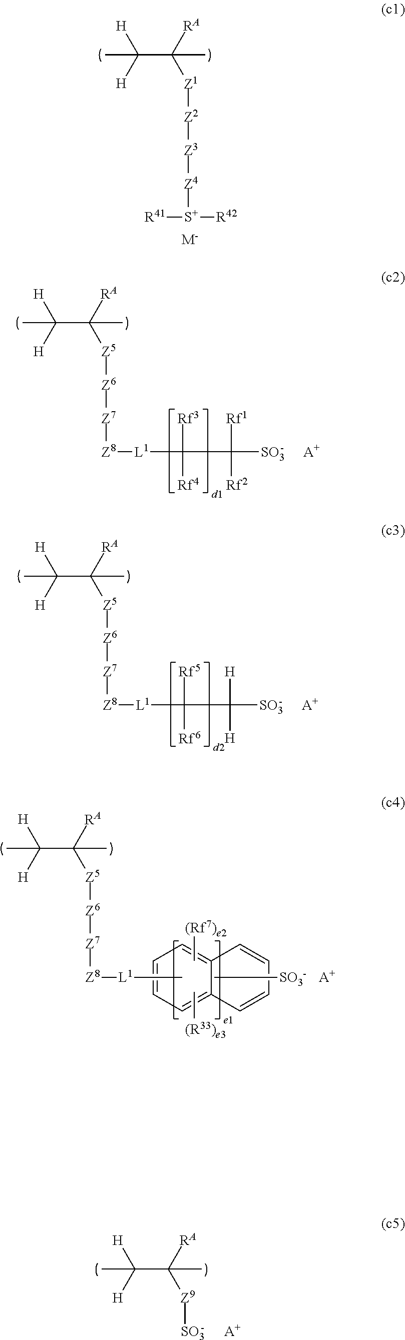

wherein “d1” and “d2” each independently represent an integer from 0 to 3;
“e1” represents 0 or 1; “e2” represents an integer from 0 to 4; “e3” represents an integer from 0 to 4; however, when “e1” represents 0, 0≤e2+e3≤4, and when “e1” represents 1, 0≤e2+e3≤6;
R^{A} each independently represents a hydrogen atom, a fluorine atom, a methyl group, or a trifluoromethyl group;
Z¹ represents a single bond or a phenylene group which may have a substituent;
Z² represents a single bond, **—C(═O)—O—Z²¹—, **—C(═O)—NH—Z²¹—, or **—O—Z²¹—; Z²¹ represents an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ represents a single bond, an ether bond, an ester bond, a sulfonate ester bond, an amide bond, a sulfonamide bond, a carbonate bond, or a carbamate bond;
Z⁴ represents a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ each independently represents a single bond, a phenylene group or naphthylene group which may have a substituent, or *—C(═O)—O—Z⁵¹—; Z⁵¹ represents an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ represents a single bond, an ether bond, an ester bond, a sulfonate ester bond, an amide bond, a sulfonamide bond, a carbonate bond, or a carbamate bond;
Z⁷ each independently represents a single bond, ***—Z⁷¹—C(O)—O—, ***—C(═O)—NH—Z⁷¹—, or ***—O—Z⁷¹; Z⁷¹ represents a hydrocarbylene group having 1 to 20 carbon atoms and optionally containing a heteroatom;
Z⁸ each independently represents a single bond, ****—Z⁸¹—C(═O)—O—, ****—C(═O)—NH—Z⁸¹—, or ****—O—Z⁸¹; Z⁸¹ represents a hydrocarbylene group having 1 to 20 carbon atoms and optionally containing a heteroatom;
Z⁹ represents a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—; Z⁹¹ represents an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
* represents an attachment point to a main chain carbon atom; “**” represents an attachment point to Z¹; “***” represents an attachment point to Z⁶; “****” represents an attachment point to Z⁷;
L¹ represents a single bond, an ether bond, an ester bond, a carbonyl group, a sulfonate ester bond, a sulfonamide bond, a carbonate bond, or a carbamate bond;
Rf¹ and Rf² each independently represent a fluorine atom or a fluorinated saturated hydrocarbyl group having 1 to 6 carbon atoms;
Rf³ and Rf⁴ each independently represent a hydrogen atom, a fluorine atom, or a fluorinated saturated hydrocarbyl group having 1 to 6 carbon atoms;
Rf⁵ and Rf⁶ each independently represent a hydrogen atom, a fluorine atom, or a fluorinated saturated hydrocarbyl group having 1 to 6 carbon atoms; however, all Rf⁵ and Rf⁶ cannot simultaneously become a hydrogen atom;
Rf⁷ represents a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² each independently represent a hydrocarbyl group having 1 to 20 carbon atoms and optionally containing a heteroatom; furthermore, R⁴¹ and R⁴² may be bonded to each other to form a ring together with the sulfur atom to which they are bonded;
R⁴³ represents a halogen atom other than a fluorine atom, or a hydrocarbyl group having 1 to 20 carbon atoms and optionally containing a heteroatom; when “e3” is 2, 3, or 4, a plurality of R⁴³ may be bonded to each other to form a ring together with the carbon atoms to which they are bonded;
M⁻ represents a non-nucleophilic counterion; and
A⁺ represents an onium cation.

In the present invention, it is preferable that the base polymer has such a repeating unit.

In addition, the chemically amplified resist composition of the present invention preferably further contains at least one constituent selected from an organic solvent, a quencher, a photoacid generator other than the above-mentioned photoacid generator, and a surfactant.

The chemically amplified resist composition of the present invention may contain these additives.

The present invention also provides a patterning process, comprising the steps of: forming a resist film on a substrate using the above-described chemically amplified resist composition; exposing the resist film with a high-energy beam; and developing the exposed resist film using a developer.

Such a patterning process can provide a patterning process using a chemically amplified resist composition that has excellent solvent solubility, high sensitivity, high contrast, and excellent lithography performance in photolithography using high-energy beams.

In this case, the high-energy beam is preferable KrF excimer laser light, ArF excimer laser light, an electron beam, or extreme ultraviolet having a wavelength of 3 to 15 nm.

The above-mentioned high-energy beams can be used.

### Advantageous Effects of Invention

When a pattern is formed using a chemically amplified resist composition containing the onium salt of the present invention as a photoacid generator, particularly in photolithography using high-energy beams such as KrF excimer laser light, ArF excimer laser light, electron beam (EB), or EUV, the composition exhibits excellent solvent solubility, high sensitivity, high contrast, and excellent acid diffusion suppression ability, and also improves lithography performance such as LWR, CDU, MEF, EL, and DOF, and can suppress collapse of the resist pattern when forming a fine pattern.

### DESCRIPTION OF EMBODIMENTS

As stated above, there have been demands for the development of: an onium salt used as a photoacid generator contained in a chemically amplified resist composition having excellent solvent solubility, high sensitivity, high contrast, and excellent lithography performance in photolithography using high-energy beams; a photoacid generator consisting of the onium salt; a chemically amplified resist composition containing the photoacid generator; and a patterning process using the chemically amplified resist composition.

As a result of earnest studies conducted by the present inventors in order to achieve the above-mentioned object, they have found that an onium salt consisting of an aromatic sulfonate anion substituted with a hydrocarbyl group containing at least one aromatic ring and a sulfonium cation containing a cyano group has excellent solvent solubility, and that a chemically amplified resist composition using this as a photoacid generator has high sensitivity, high contrast, is extremely effective for suppressing acid diffusion, is excellent in terms of lithography performance such as LWR, CDU, MEF, EL, DOF, etc., and is extremely effective for forming fine patterns. This has led to the completion of the present invention.

That is, the present invention is an onium salt, wherein the onium salt comprises an anion represented by the following general formula (1A) and a cation represented by the following general formula (1B),

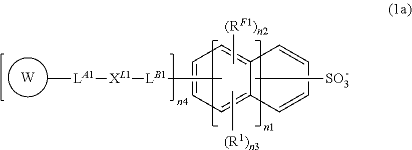

wherein “n1” represents 0 or 1; “n2” represents an integer from 0 to 4; “n3” represents an integer from 0 to 4; however, when “n1” represents 0, 0≤n2+n3≤4, and when “n1” represents 1, 0≤n2+n3≤6; “n4” represents 0 or 1;
W represents a hydrocarbyl group having 6 to 60 carbon atoms and containing at least one aromatic ring, and the hydrocarbyl group may contain a heteroatom;
R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” represents 2, 3, or 4, each R¹ may be the same as or different from each other, and a plurality of R¹ may be bonded to each other to form a ring together with the carbon atoms to which they are bonded;
L^{A1} and L^{B1} each independently represent a single bond, an ether bond, an ester bond, a sulfonate ester bond, an amide bond, a sulfonamide bond, a carbonate bond, or a carbamate bond; and
X^{L1} represents a single bond or a hydrocarbylene group having 1 to 40 carbon atoms and optionally containing a heteroatom;

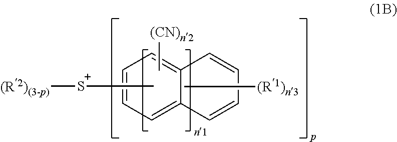

wherein “p” represents 1, 2, or 3; “n′1” represents 0 or 1; “n′2” represents 1 or 2; “n′3” represents an integer from 0 to 6; however, when “n′1” represents 0, 1≤n′2+n′3≤5, and when “n′1” represents 1, 1≤n′2+n′3≤7;
R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n′3” represents 2 to 6, each R′¹ may be the same as or different from each other, and two R′¹ may be bonded to each other to form a ring together with the carbon atoms to which they are bonded;
R′² represents a halogen atom or a hydrocarbyl group having 1 to 30 carbon atoms and optionally containing a heteroatom; when “p” represents 1, two R′² may be the same as or different from each other; and two of the three substituents bonded to S⁺ may be bonded to each other to form a ring together with the sulfur atom to which they are bonded.

Hereinafter, the present invention will be described in detail, but the present invention is not limited thereto. In the following explanation, some structures represented by chemical formulae may have asymmetric carbons and may have enantiomers or diastereomers; in such cases, one formula will be used to represent all of the isomers. These isomers may be used alone or in a combination of two or more types.

### [Onium Salt]

The onium salt of the present invention consists of an anion represented by the following general formula (1A) and a cation represented by the following general formula (1B).

The anion contained in the onium salt of the present invention is represented by the following general formula (1A).

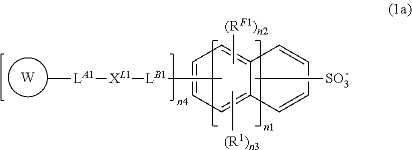

In the formula, “n1” represents 0 or 1; “n2” represents an integer from 0 to 4; “n3” represents an integer from 0 to 4; however, when “n1” represents 0, 0≤n2+n3≤4, and when “n1” represents 1, 0≤n2+n3≤6; “n4” represents 0 or 1;
W represents a hydrocarbyl group having 6 to 60 carbon atoms and containing at least one aromatic ring, and the hydrocarbyl group may contain a heteroatom;
R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” represents 2, 3, or 4, each R¹ may be the same as or different from each other, and a plurality of R¹ may be bonded to each other to form a ring together with the carbon atoms to which they are bonded;
L^{A1} and L^{B1} each independently represent a single bond, an ether bond, an ester bond, a sulfonate ester bond, an amide bond, a sulfonamide bond, a carbonate bond, or a carbamate bond; and
X^{L1} represents a single bond or a hydrocarbylene group having 1 to 40 carbon atoms and optionally containing a heteroatom.

In the general formula (1A), “n1” represents 0 or 1. When “n1” is 0, the structure is a benzene ring, and when “n1” is 1, it is a naphthalene ring; however, from the viewpoint of solvent solubility, it is preferable that “n1” is 0, giving a benzene ring. “n2” represents an integer from 0 to 4. From the viewpoint of raw material procurement, when “n2” is 1 or more, it is preferable that “n2” is 4. “n3” represents an integer from 0 to 4. However, when “n1” is 0, 0≤n2+n3≤4, and when “n1” is 1, 0≤n2+n3≤6. “n4” represents 0 or 1, but from the viewpoint of acid diffusion control, “n4” is preferably 1.

In the general formula (1A), R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. R^{F1} preferably represents a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. The inclusion of a fluorine atom or one of these substituents having a fluorine atom improves the acid strength of the generated acid through an electron-withdrawing effect, allowing the deprotection reaction of acid-labile groups such as tertiary esters and tertiary ethers, which will be described later, to proceed smoothly. When “n2” is 2, 3, or 4, each R^{F1} may be the same as or different from each other.

In the general formula (1A), R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Examples of halogen atoms other than fluorine atoms include chlorine atoms, bromine atoms, iodine atoms, etc., with iodine atoms being preferable. The hydrocarbyl group may be saturated or unsaturated and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, allyl, propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl and naphthyl groups; aralkyl groups having 7 to 20 carbon atoms, such as benzyl, 1-phenylethyl, and 2-phenylethyl groups; and groups obtained by combining these, etc. Of these, aryl groups are preferable. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. When “n3” is 2, 3, or 4, each R¹ may be the same as or different from each other. Also, when “n3” is 2, 3, or 4, a plurality of R¹ may be bonded to each other to form a ring together with the carbon atoms to which they are bonded. The ring is preferably a 5- to 8-member ring.

In the general formula (1A), W is a hydrocarbyl group containing at least one aromatic ring and having 6 to 60 carbon atoms, and preferably 6 to 40 carbon atoms, and the hydrocarbyl group may contain a heteroatom.

In the general formula (1A), the W is preferably represented by the following general formula (W-1) or (W-2),

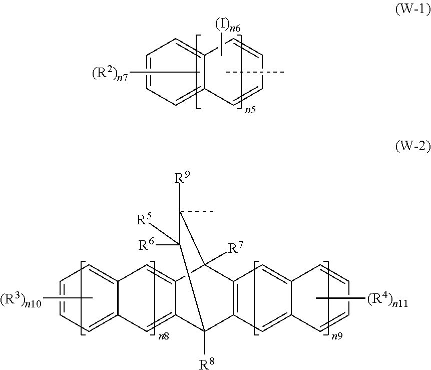

In the formulae, “n5” represents 0 or 1; “n6” represents an integer from 0 to 4; “n7” represents an integer from 1 to 4; however, when “n5” represents 0, 1≤n6+n7≤5, and when “n5” represents 1, 1≤n6+n7≤7; “n8” represents 0 or 1; “n9” represents 0 or 1; “n10” represents an integer from 0 to 4; “n11” represents an integer from 0 to 4;
R² each independently represents a hydrogen atom, a halogen atom other than an iodine atom, or a hydrocarbyl group having 1 to 20 carbon atoms and optionally containing a heteroatom;
R³ and R⁴ each independently represent a hydrogen atom, a halogen atom, or a hydrocarbyl group having 1 to 20 carbon atoms and optionally containing a heteroatom;
R⁵ to R⁹ each independently represent a hydrogen atom, a halogen atom, or a hydrocarbyl group having 1 to 20 carbon atoms and optionally containing a heteroatom; and
a broken line represents an attachment point to L^{A1}.

In the general formula (W-1), “n5” represents 0 or 1. When “n5” is 0, the structure is a benzene ring, and when “n5” is 1, it is a naphthalene ring; however, from the viewpoint of solvent solubility, it is preferable that “n5” is 0, giving a benzene ring. “n6” represents an integer from 0 to 4. When “n5” is 0, “n6” is preferably 2, 3, or 4, and from the viewpoint of EUV light absorption, “n6” is preferably 3 or 4. “n7” represents an integer from 1 to 4. From the viewpoint of raw material procurement, “n7” is preferably 1 to 3, and from the viewpoint of acid diffusion control, “n7” is preferably 2 or 3. However, when “n5” is 0, 1≤n6+n7≤5, and when “n5” is 1, 1≤n6+n7≤7.

In the general formula (W-1), R² each independently represents a hydrogen atom, a halogen atom other than an iodine atom, or a hydrocarbyl group having 1 to 20 carbon atoms and optionally containing a heteroatom. Examples of halogen atoms other than iodine atoms include fluorine atoms, chlorine atoms, and bromine atoms, with fluorine atoms being preferable from the viewpoint of solvent solubility. Furthermore, specific examples of the hydrocarbyl group having 1 to 20 carbon atoms and optionally containing a heteroatom include the same ones as those given as an example of the hydrocarbyl group represented by R¹ in the explanation of the general formula (1A), but are not limited to these. R² preferably represents a group having a branched structure or a cyclic structure. The maximum number of carbon atoms in the general formula (W-1) is 60.

In the general formula (W-1), at least one selected from R² and an iodine atom is preferably bonded to a carbon atom adjacent to the carbon atom to which L^{A1} is bonded. As a result, the rotation between the aromatic ring to which these are bonded and the aromatic ring to which the sulfo group is bonded is suppressed by steric hindrance around the bond axis of -L^{A1}-X^{L1}-L^{B1}-, thereby reducing acid diffusion.

In the general formula (W-2), “n8” represents 0 or 1. When “n8” is 0, the structure is a benzene ring, and when “n8” is 1, it is a naphthalene ring; however, from the viewpoint of solvent solubility, it is preferable that “n8” is 0, giving a benzene ring. “n9” represents 0 or 1. When “n9” is 0, the structure is a benzene ring, and when “n9” is 1, it is a naphthalene ring; however, from the viewpoint of solvent solubility, it is preferable that “n9” is 0, giving a benzene ring. “n10” represents an integer from 0 to 4. From the viewpoint of raw material procurement, “n10” is preferably 0, 1, or 2. “n11” represents an integer from 0 to 4. From the viewpoint of raw material procurement, “n11” is preferably 0, 1, or 2.

In the general formula (W-2), R³ and R⁴ each independently represent a hydrogen atom, a halogen atom, or a hydrocarbyl group having 1 to 20 carbon atoms and optionally containing a heteroatom. The halogen atom includes a fluorine atom, a chlorine atom, a bromine atom, an iodine atom, etc., and is preferably a fluorine atom or an iodine atom. Furthermore, specific examples of the hydrocarbyl group having 1 to 20 carbon atoms and optionally containing a heteroatom include the same ones as those given as an example of the hydrocarbyl group represented by R¹ in the explanation of the general formula (1A), but are not limited to these. R³ and R⁴ preferably represent a group having a branched structure or a cyclic structure.

In the general formula (W-2), R⁵ to R⁹ each independently represent a hydrogen atom, a halogen atom, or a hydrocarbyl group having 1 to 20 carbon atoms and optionally containing a heteroatom. Examples of the halogen atom include a fluorine atom, a chlorine atom, a bromine atom, and an iodine atom, with a fluorine atom and an iodine atom being preferable. Furthermore, specific examples of the hydrocarbyl group having 1 to 20 carbon atoms and optionally containing a heteroatom include the same ones as those given as an example of the hydrocarbyl group represented by R¹ in the explanation of the general formula (1A), but are not limited to these. R⁵ to R⁹ preferably represent a group having a branched structure or a cyclic structure.

In the general formula (W-2), any two of R⁵ to R⁹ may be bonded to each other to form a ring together with the carbon atoms to which they are bonded. The ring is preferably a 5- to 8-member ring. The maximum number of carbon atoms in the general formula (W-2) is 60.

In the general formula (1A), L^{A1} and L^{B1} each independently represent a single bond, an ether bond, an ester bond, a sulfonate ester bond, an amide bond, a sulfonamide bond, a carbonate bond, or a carbamate bond. Among these, a single bond, an ether bond, or an ester bond is preferable.

In the general formula (1A), X^{L1} represents a single bond or a hydrocarbylene group having 1 to 40 carbon atoms and optionally containing a heteroatom. The hydrocarbylene group may be either linear, branched, or cyclic, and specific examples thereof include an alkanediyl group, a cyclic saturated hydrocarbylene group, an arylene group, etc. Specific examples of the heteroatom include an oxygen atom, a nitrogen atom, a sulfur atom, etc.

Specific examples of the hydrocarbylene group having 1 to 40 carbon atoms and optionally containing a heteroatom represented by X^{L1}, include, but are not limited to, the following. Note that in the formulae below, * represents an attachment point to L^{A1} and L^{B1} respectively.

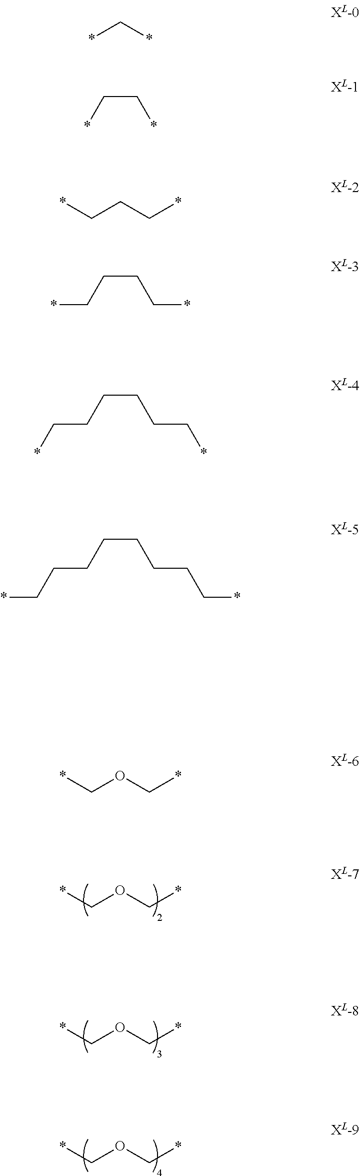

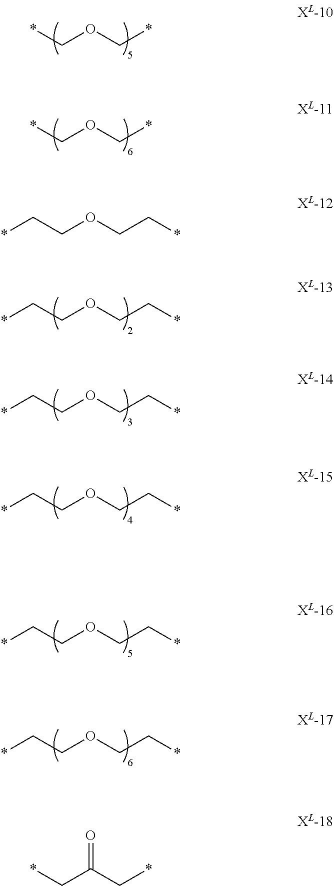

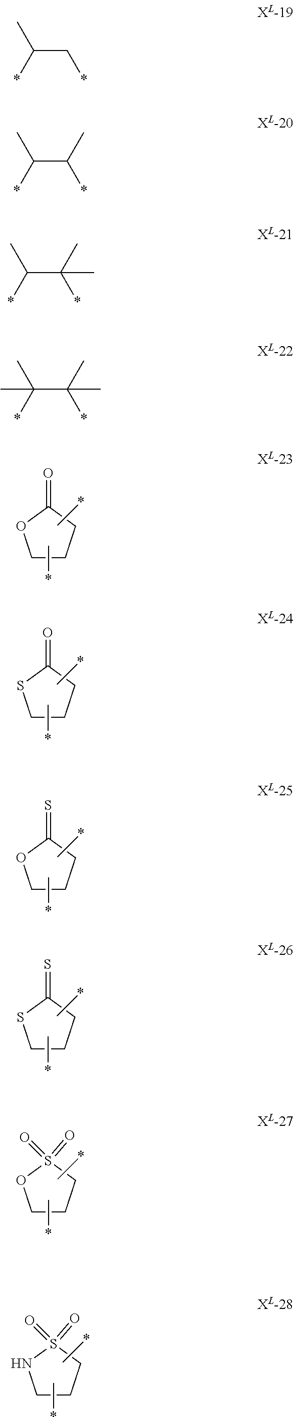

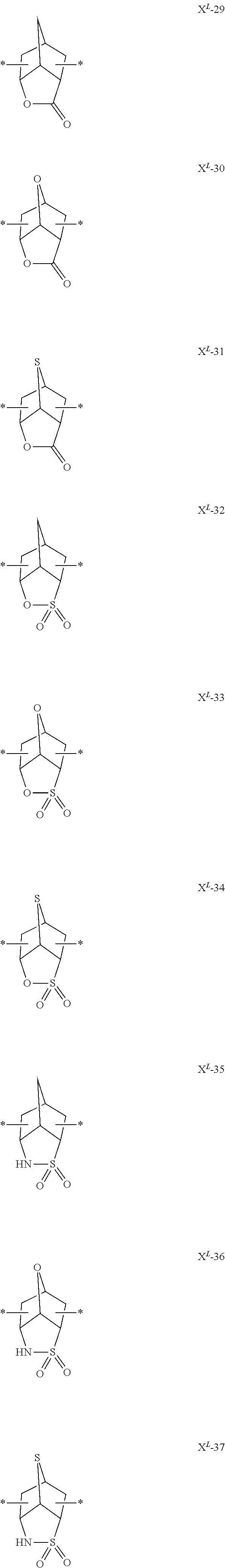

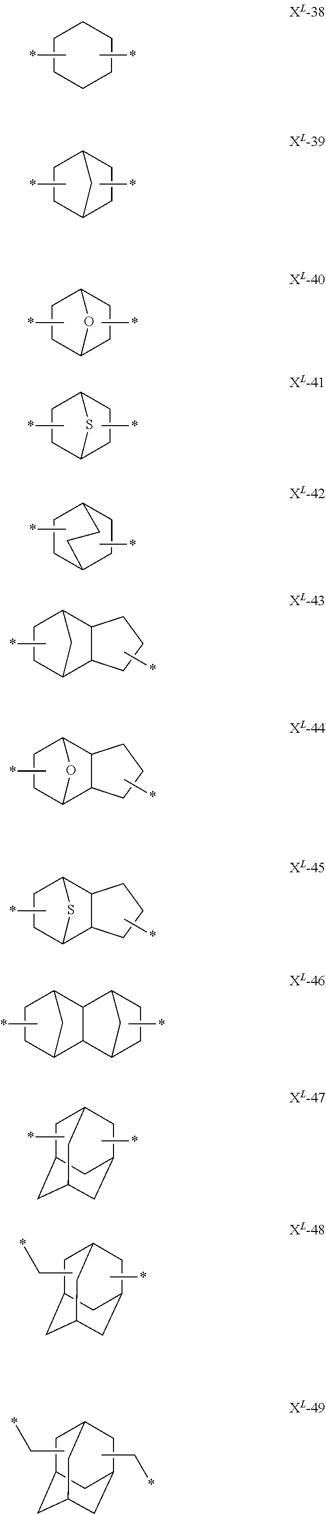

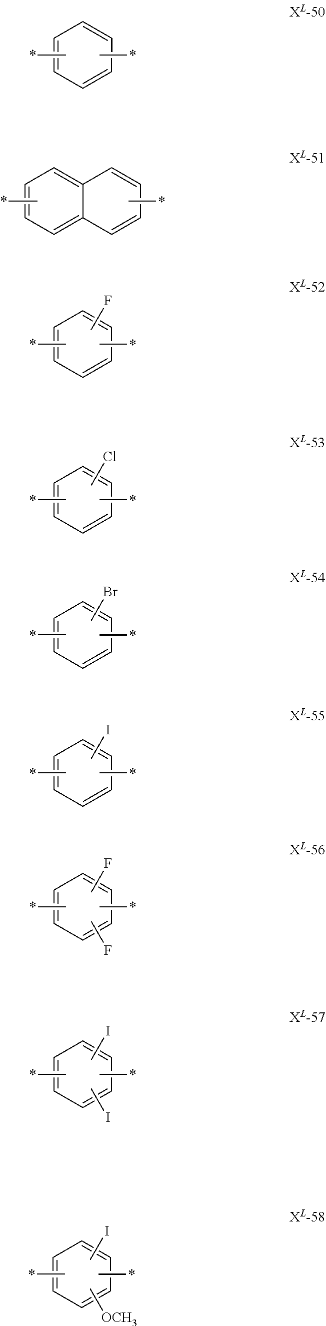

Of these, X^{L}-0 to X^{L}-22 and X^{L}-47 to X^{L}-58 are preferable.

The anion represented by the general formula (1A) is preferably represented by the following general formula (1A-1),

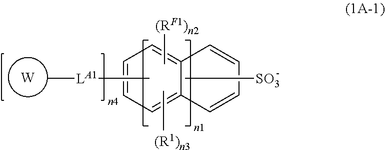

In the formula, “n1” to “n4”, W, R^{F1}, R¹, and L^{A1} are as defined above.

Specific examples of the anion represented by the general formula (1A) include, but are not limited to, the following. Note that in the following formulae, Me represents a methyl group.

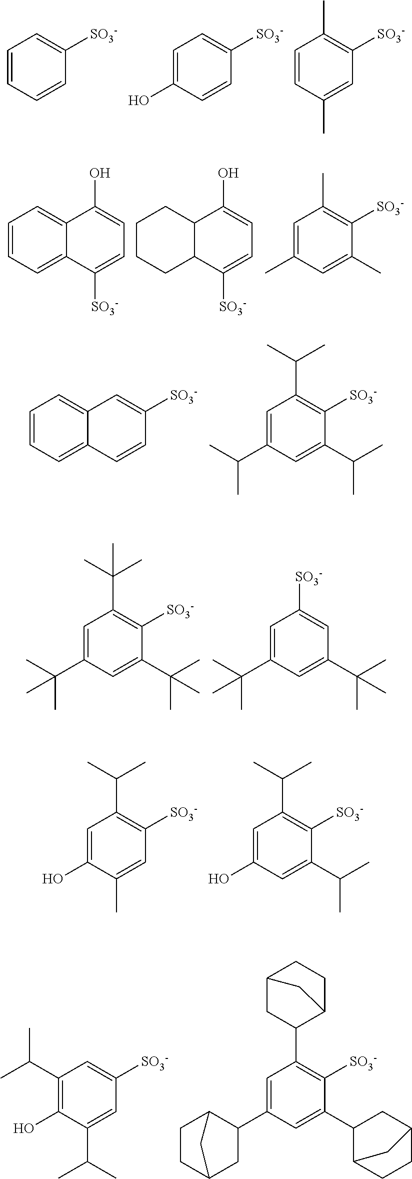

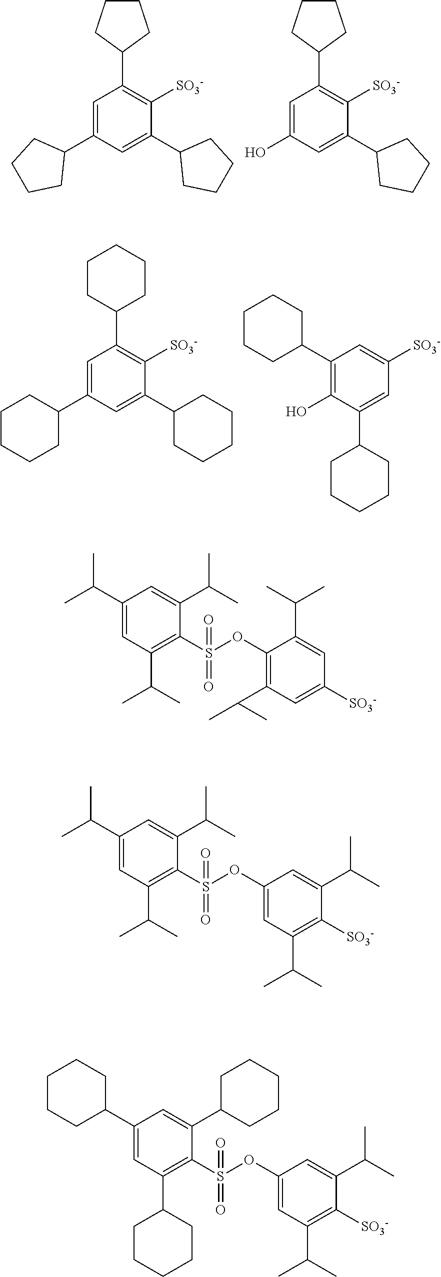

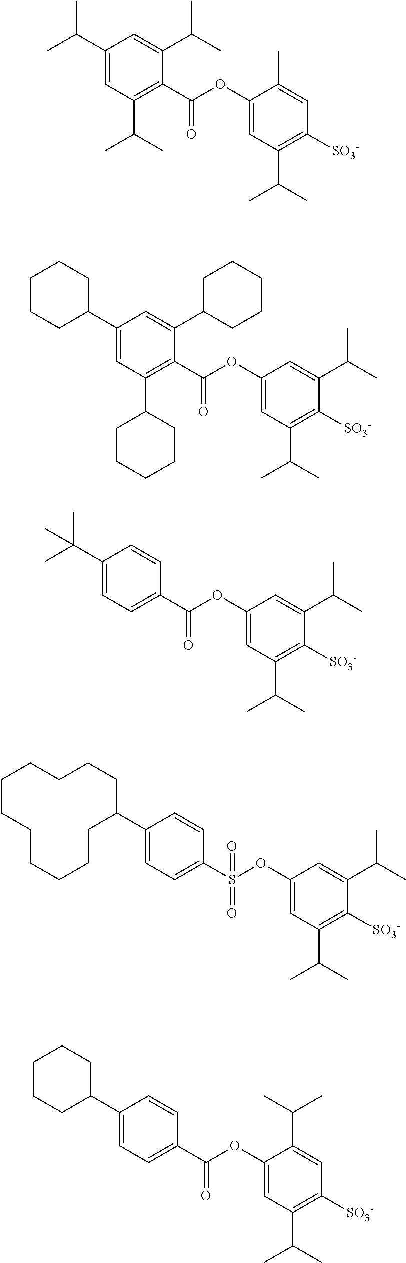

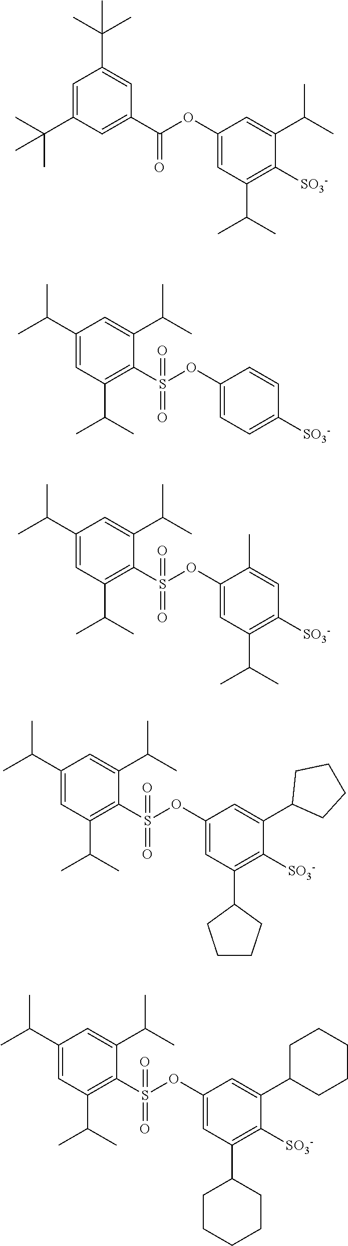

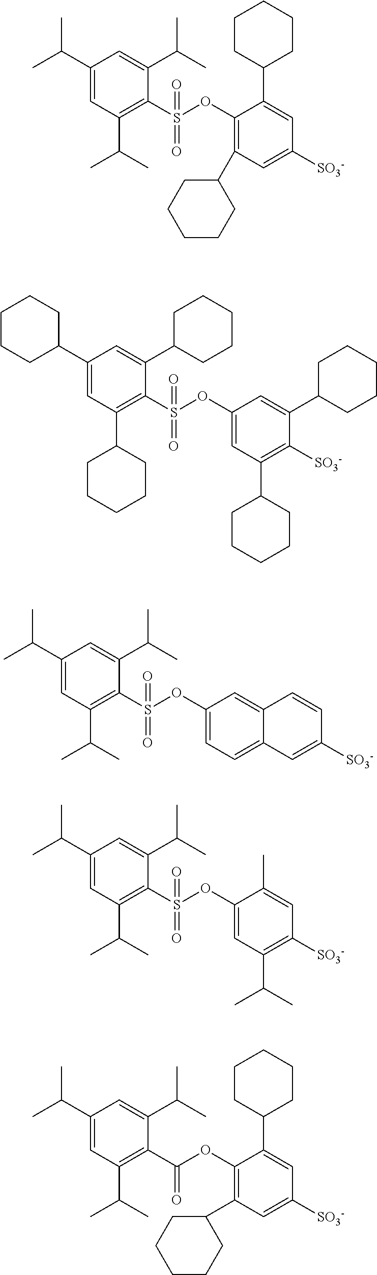

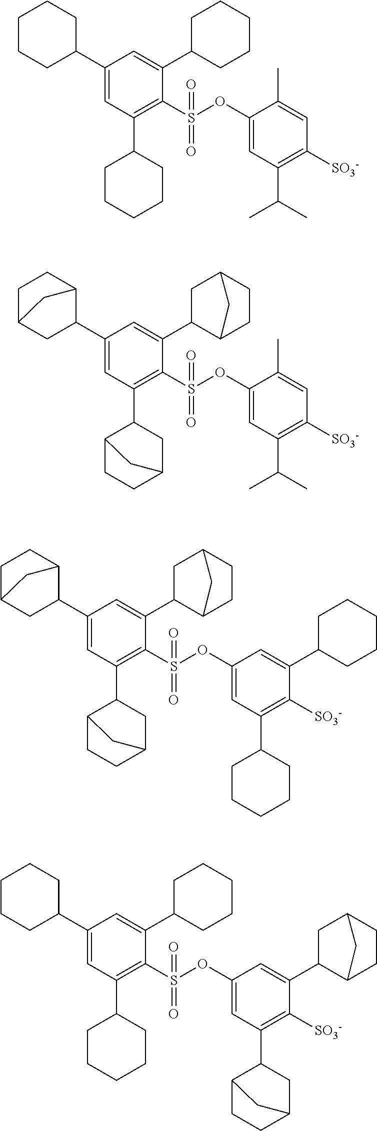

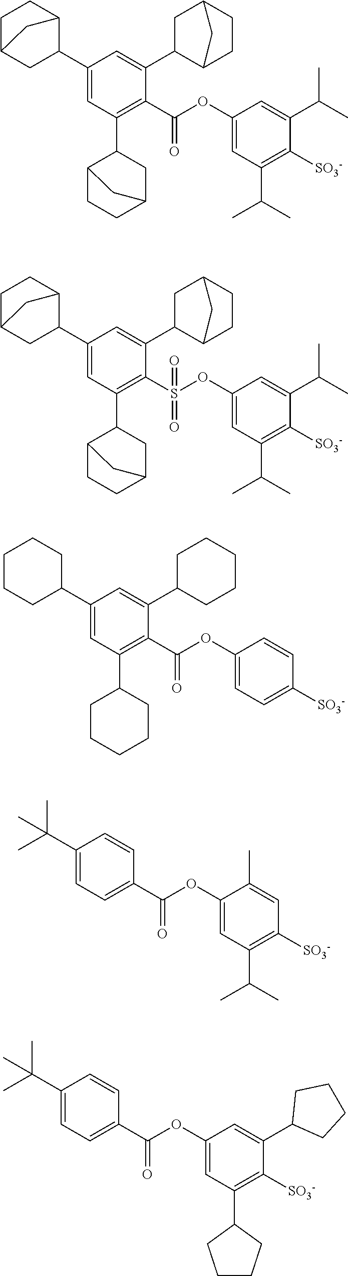

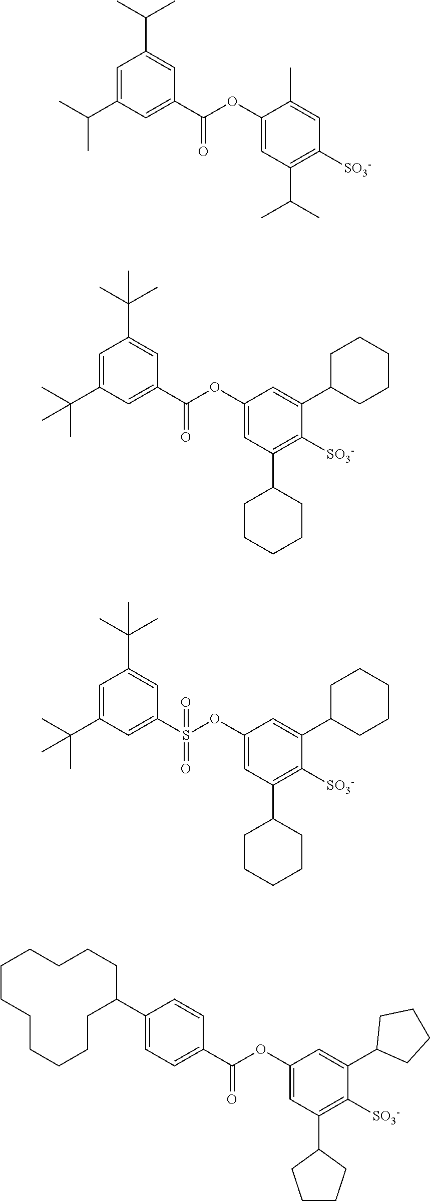

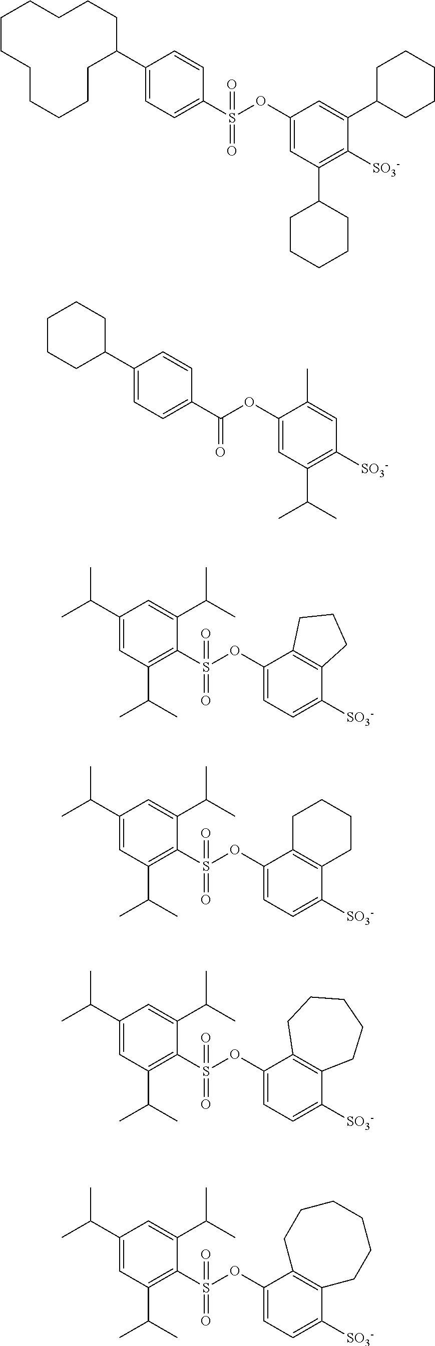

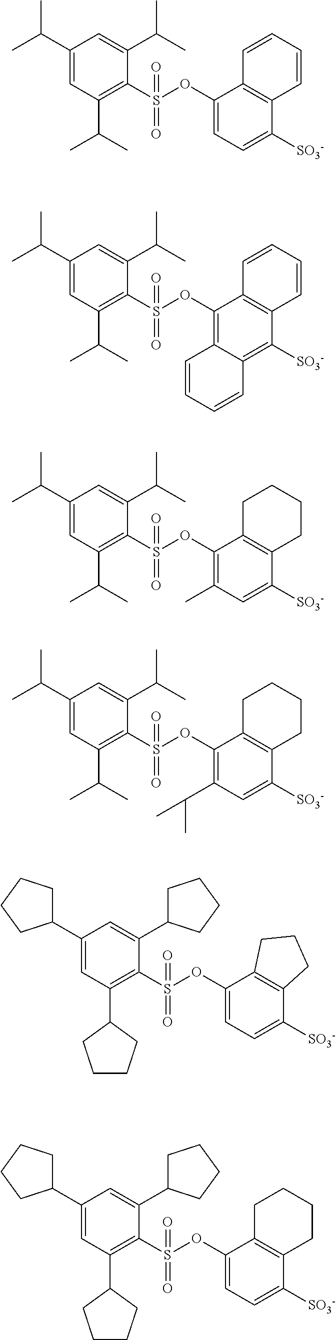

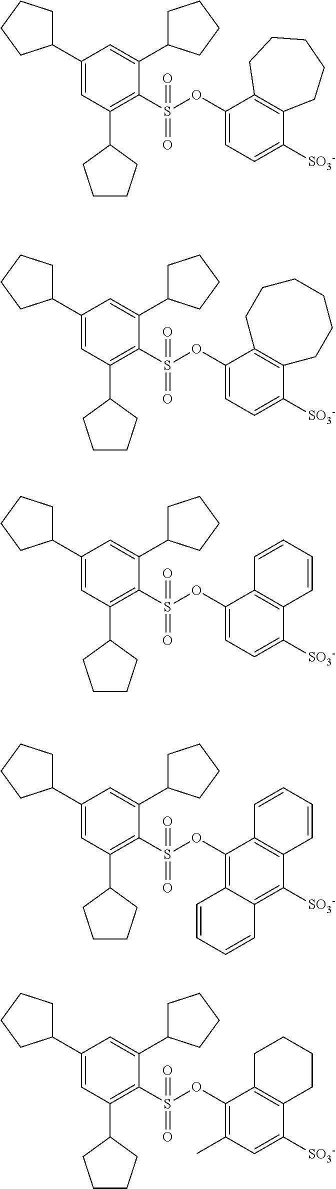

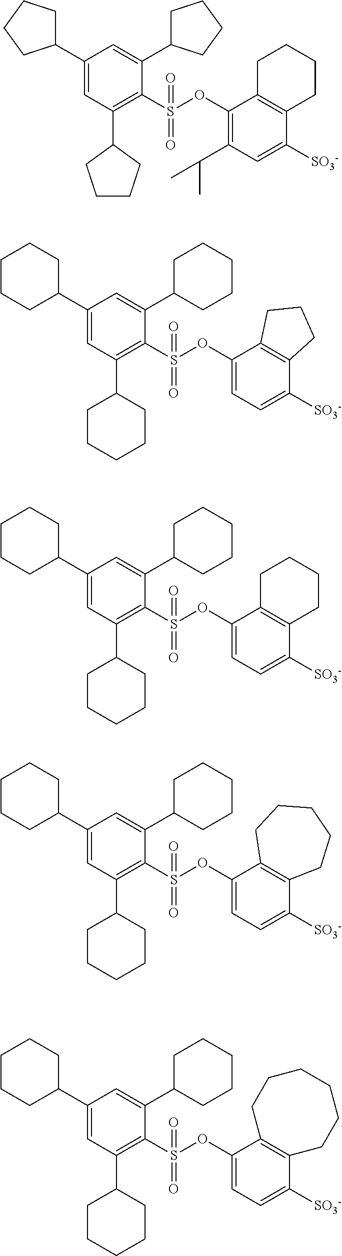

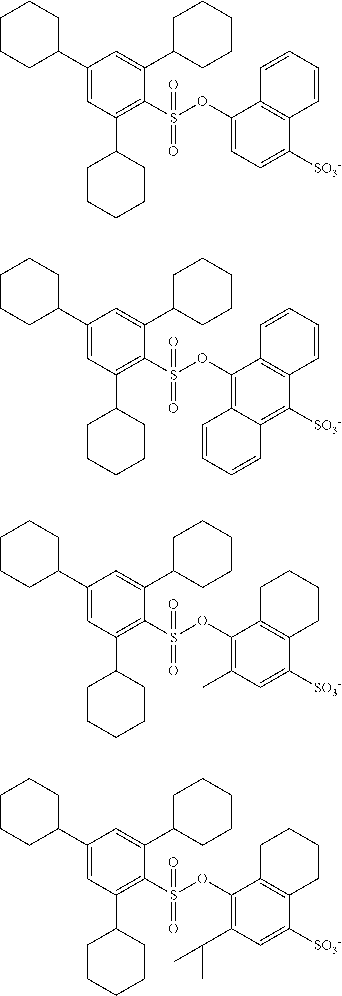

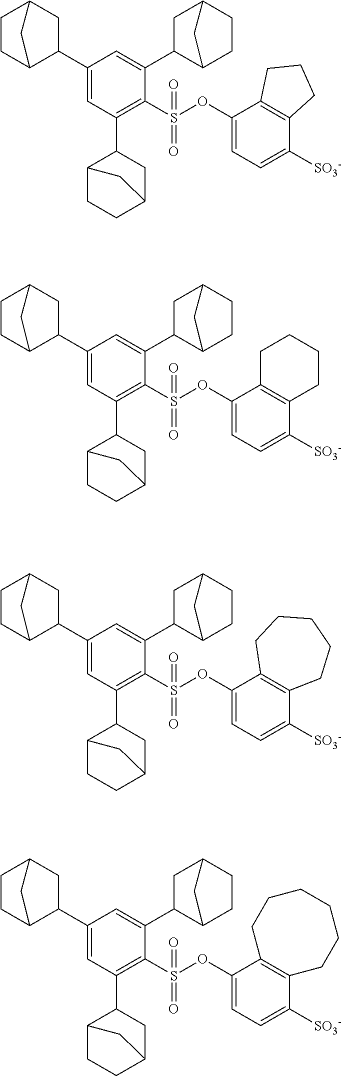

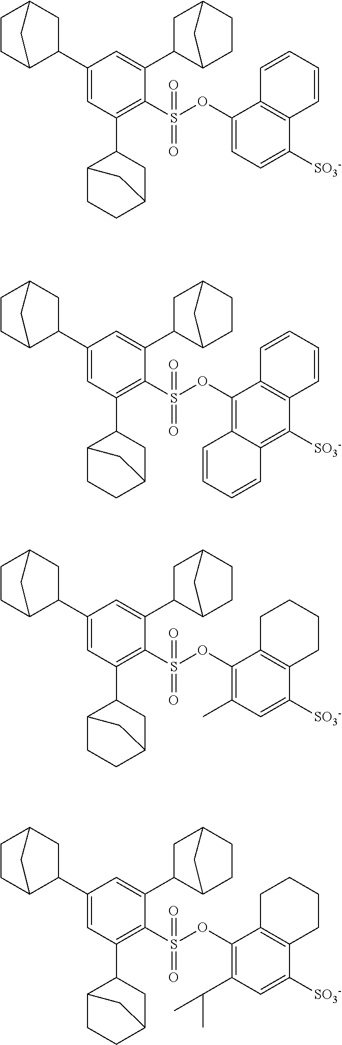

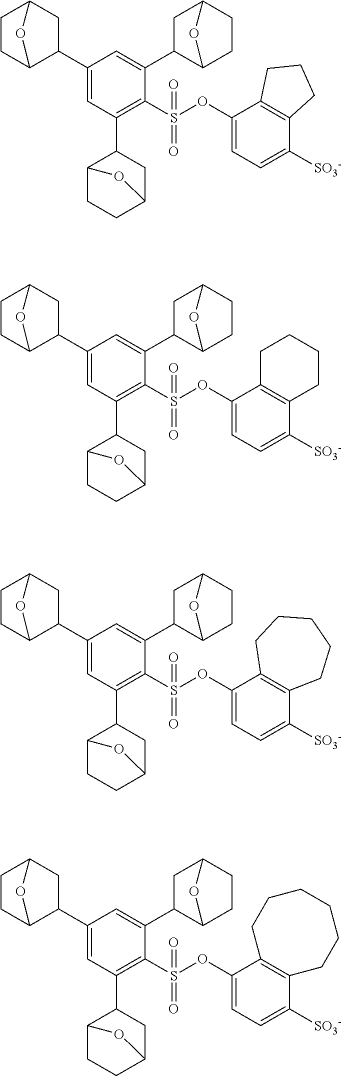

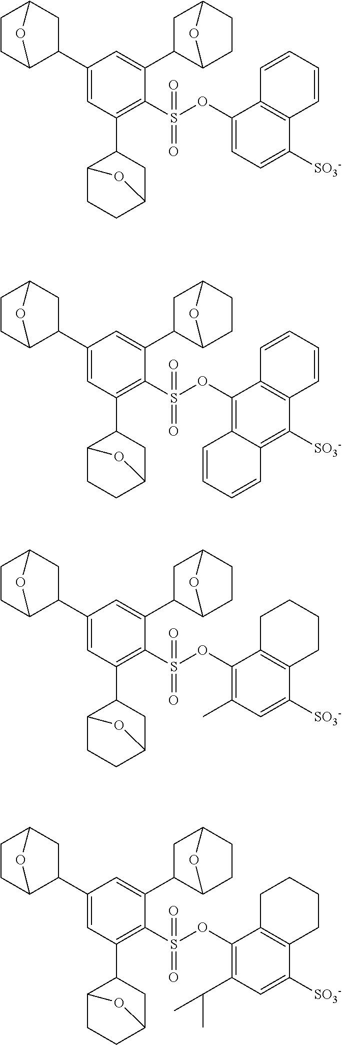

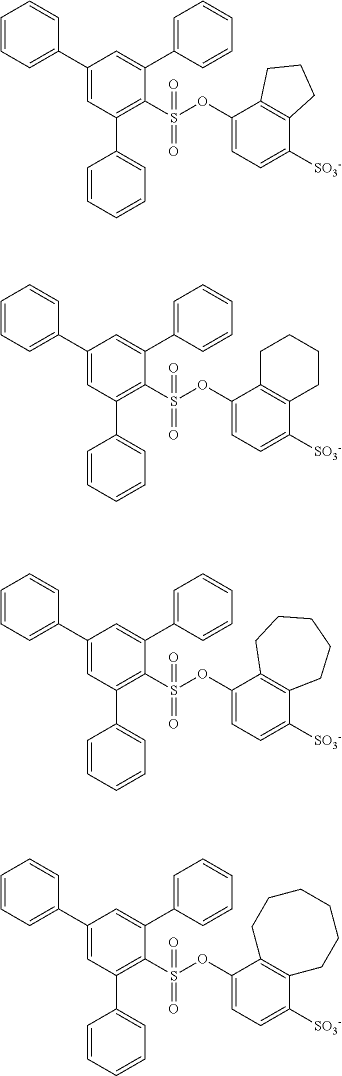

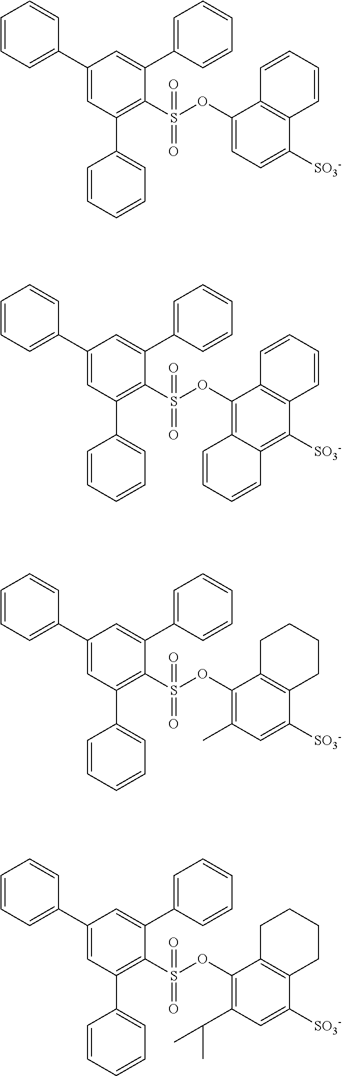

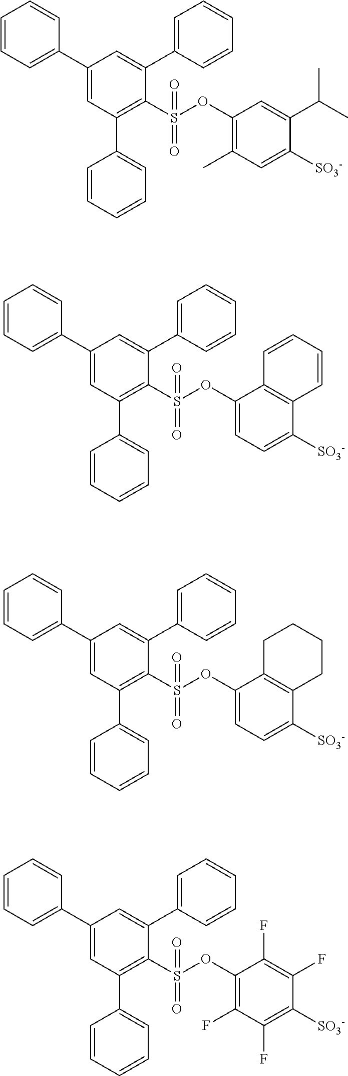

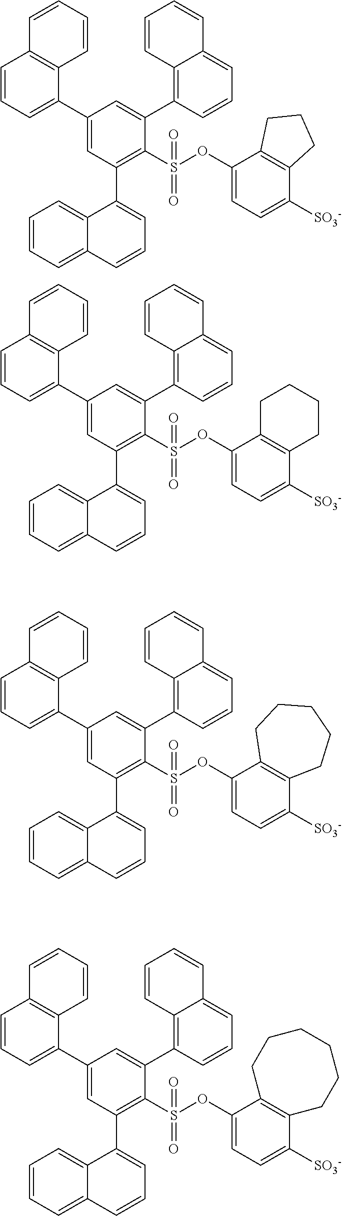

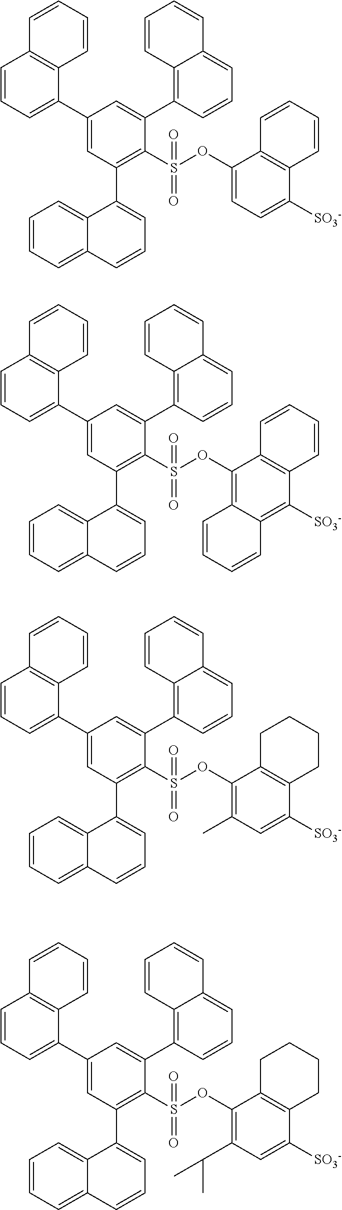

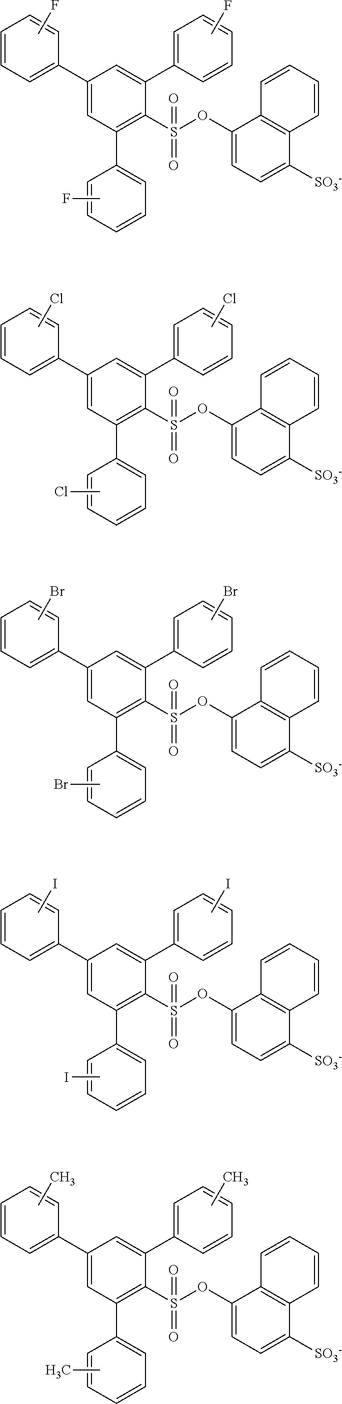

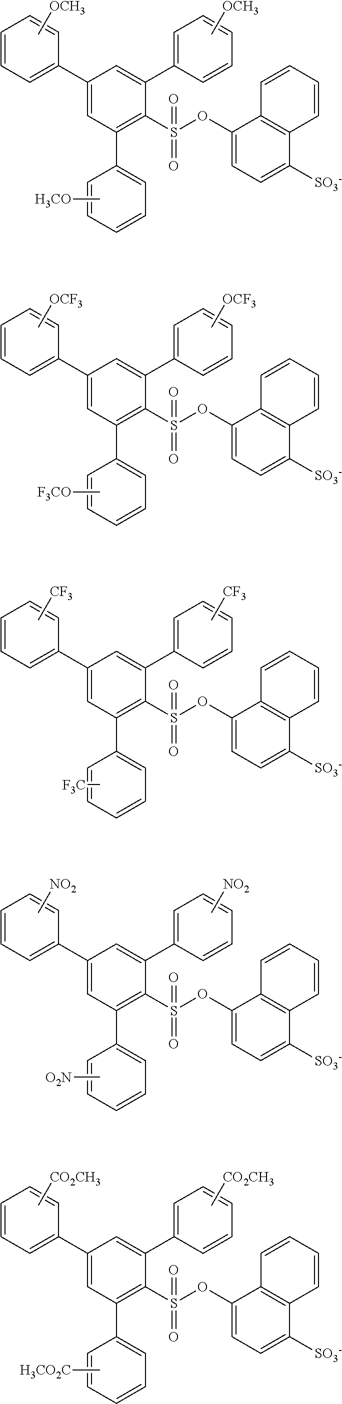

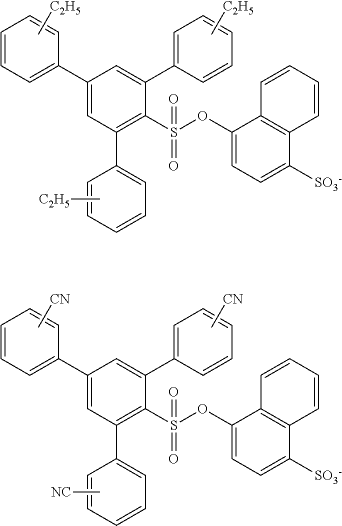

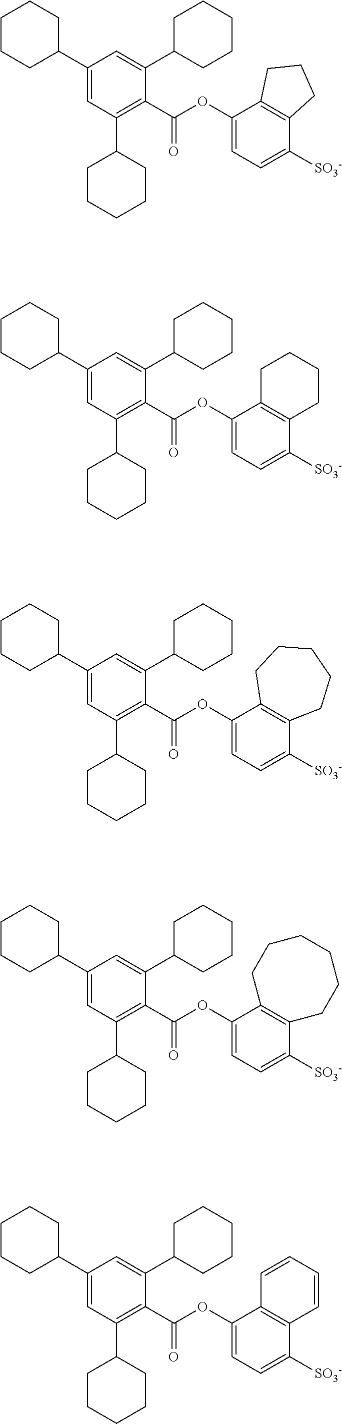

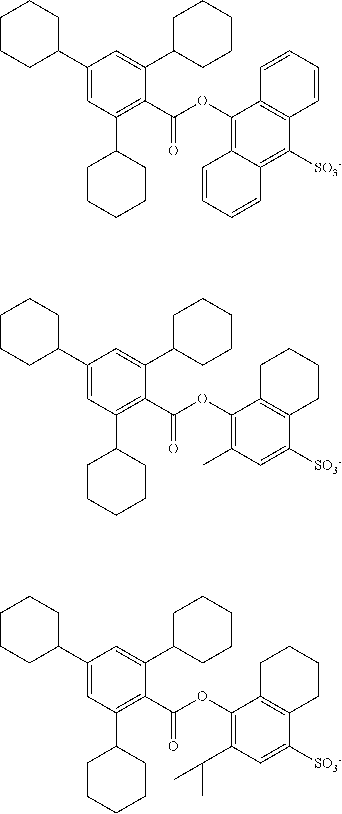

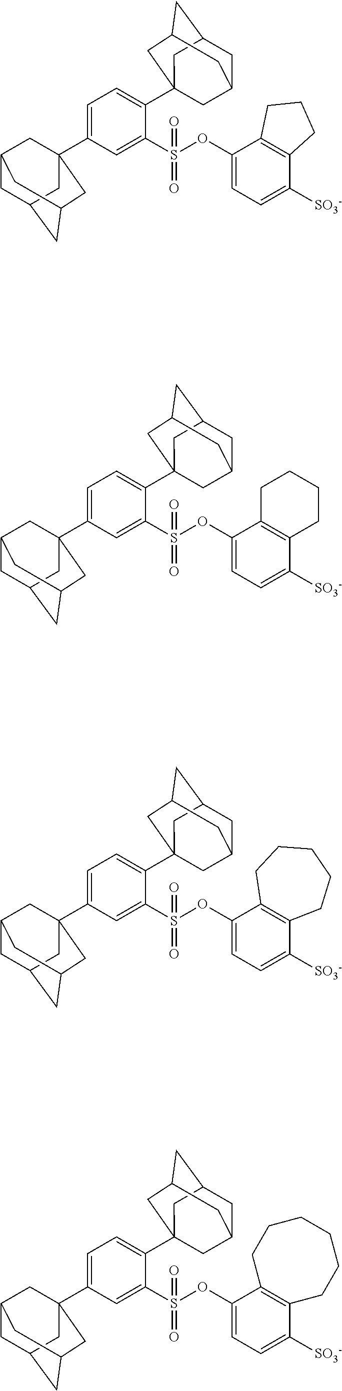

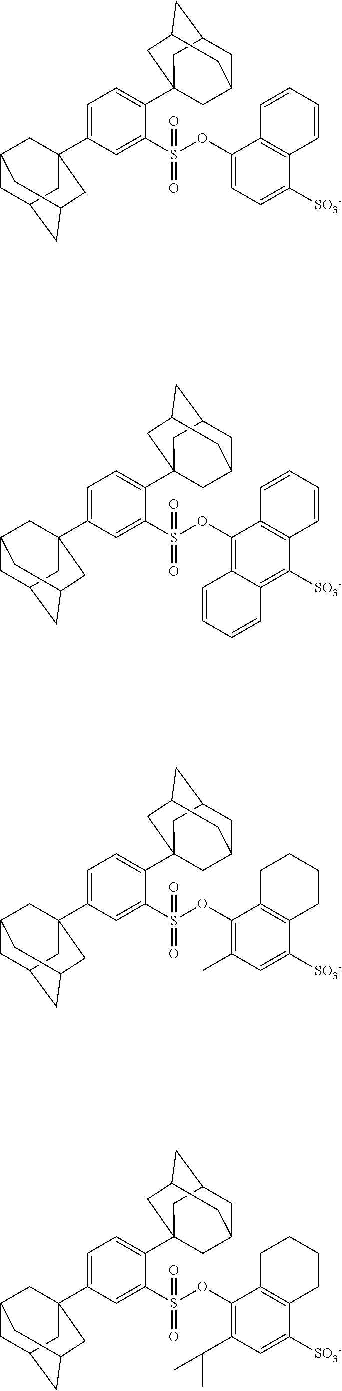

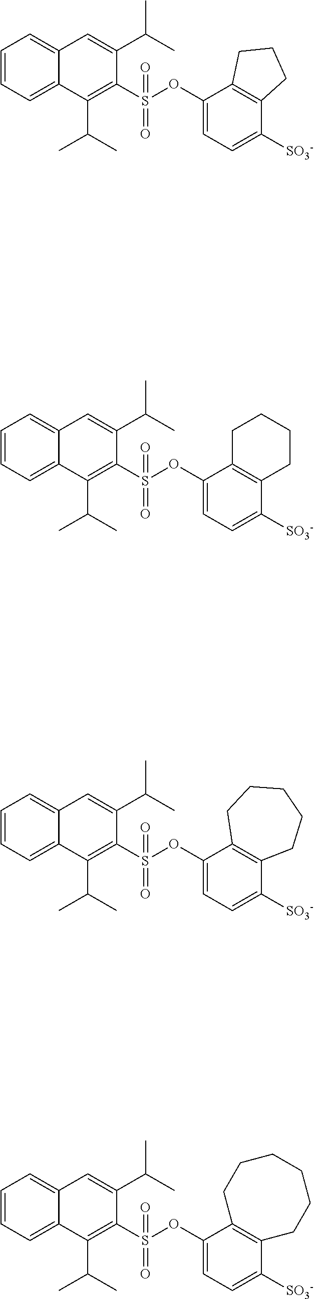

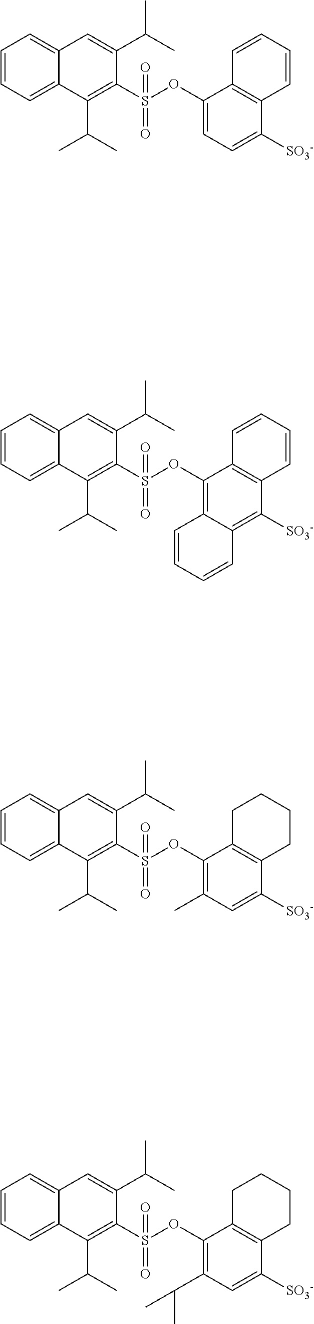

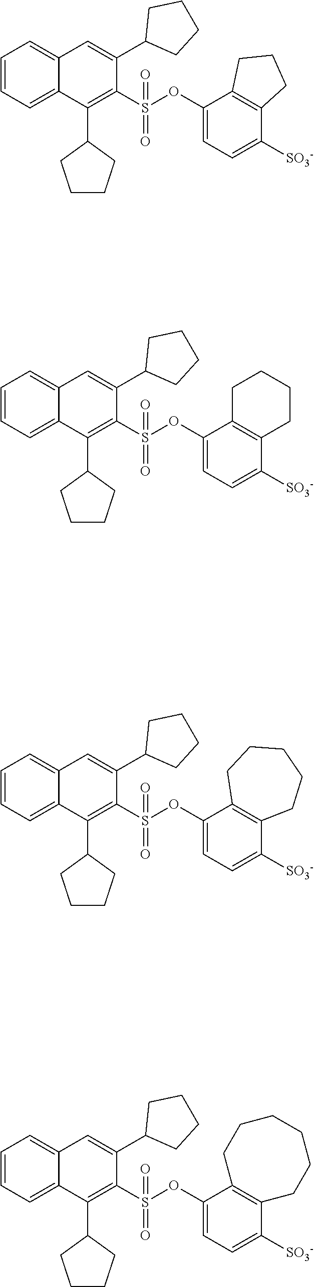

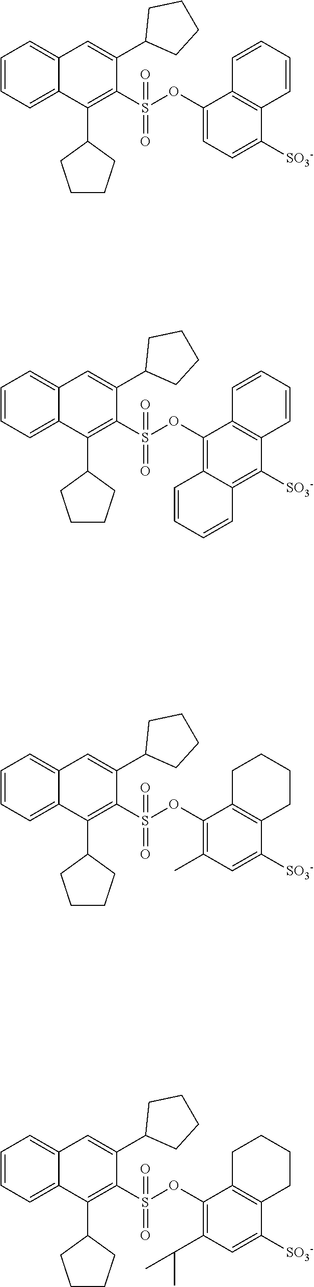

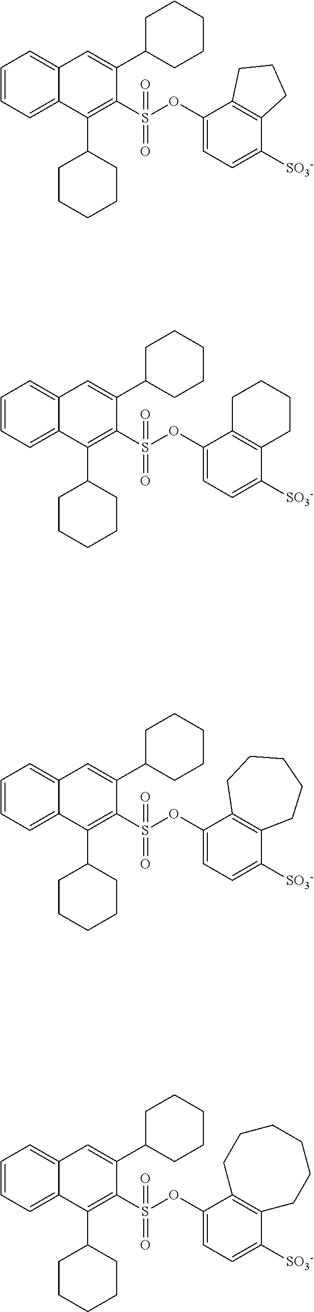

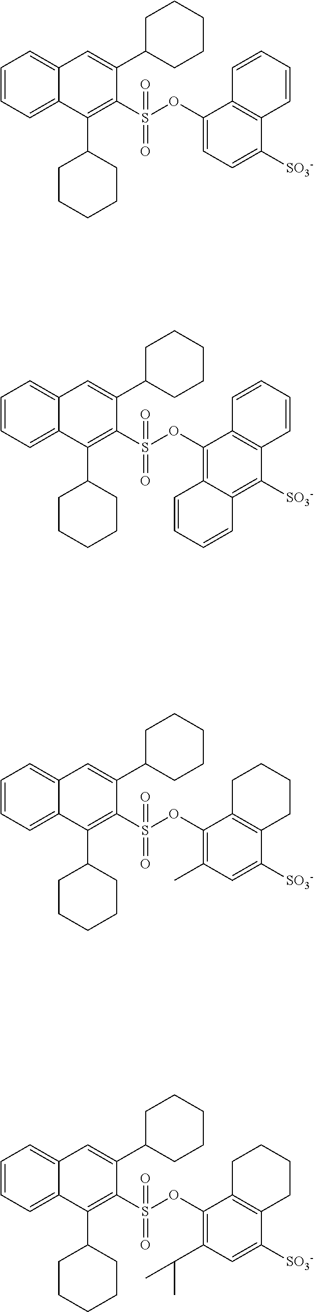

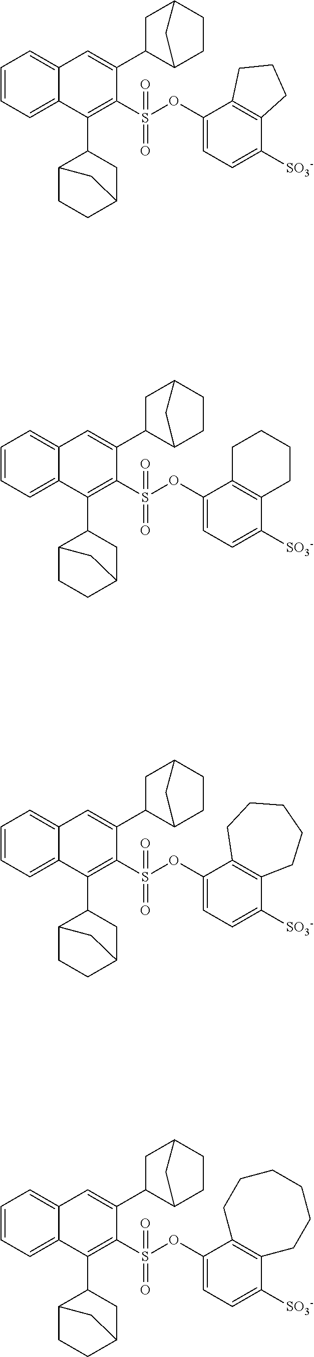

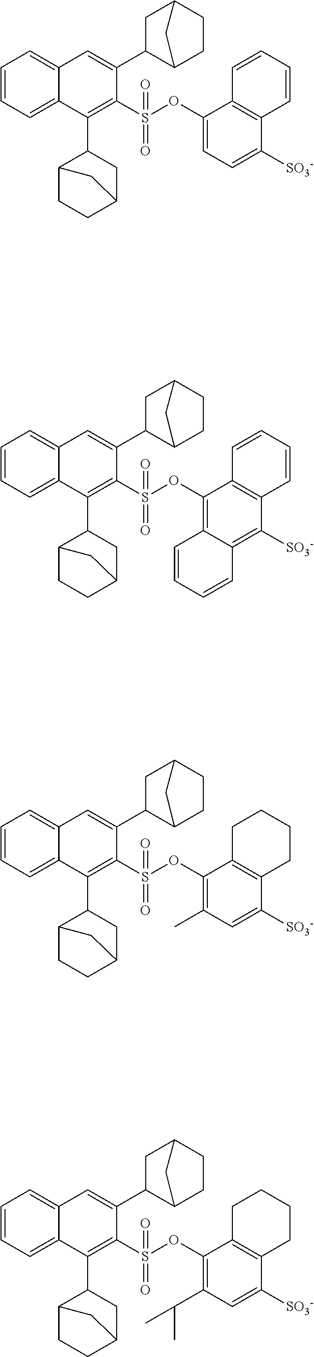

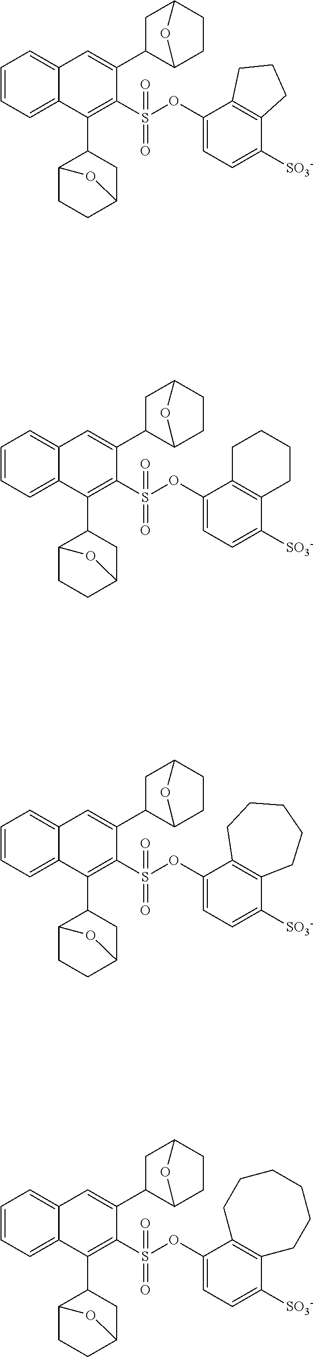

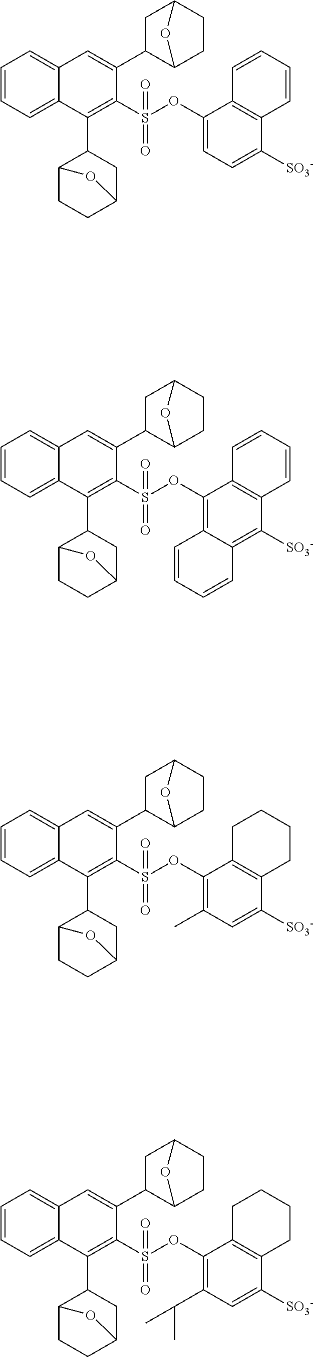

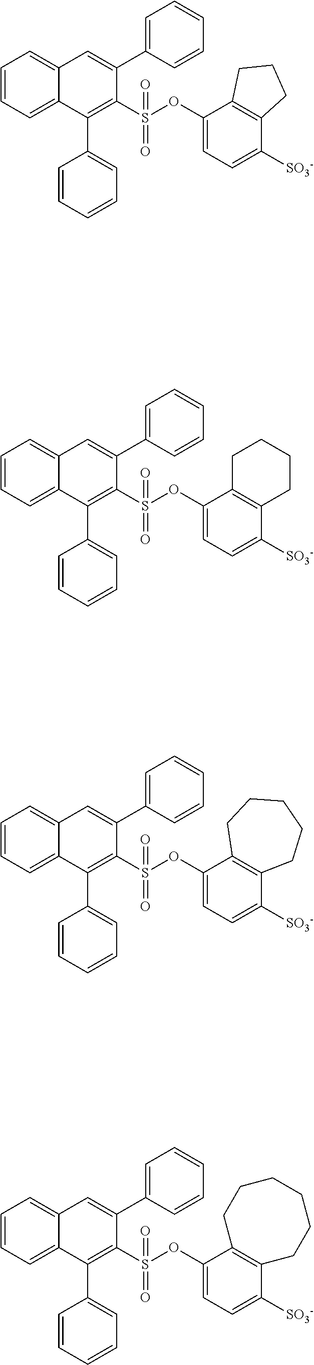

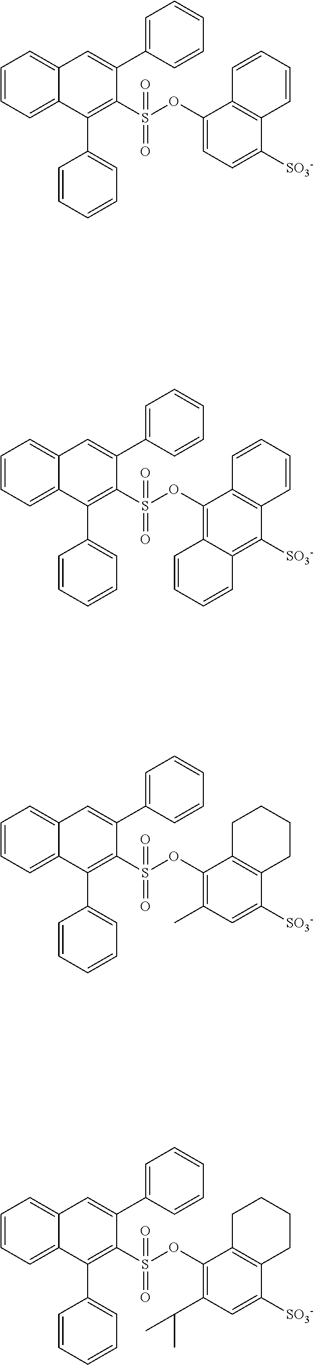

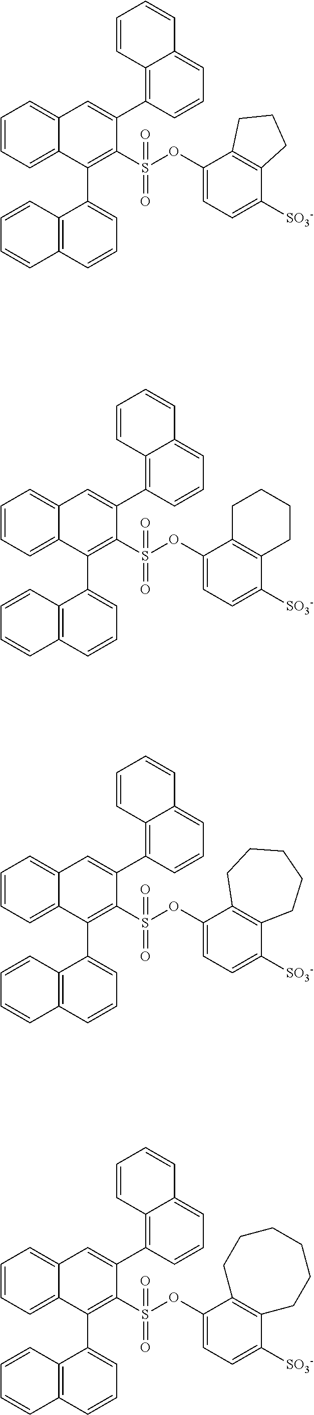

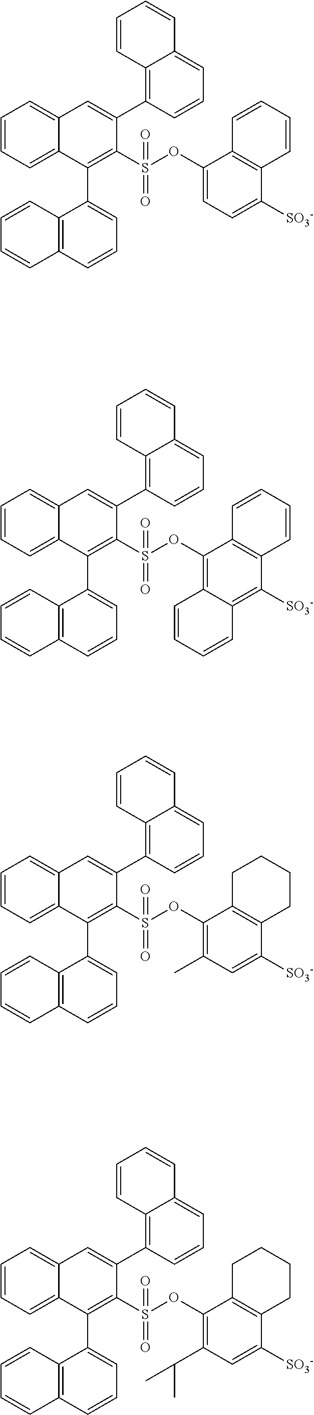

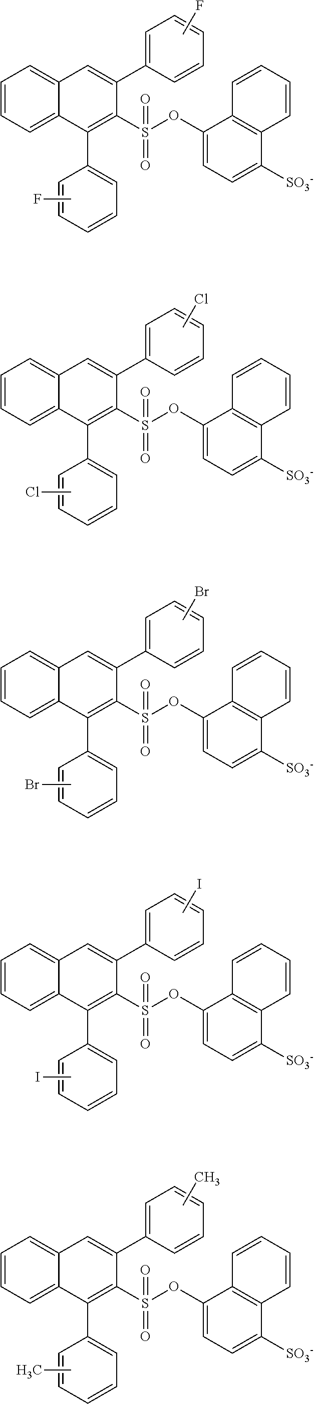

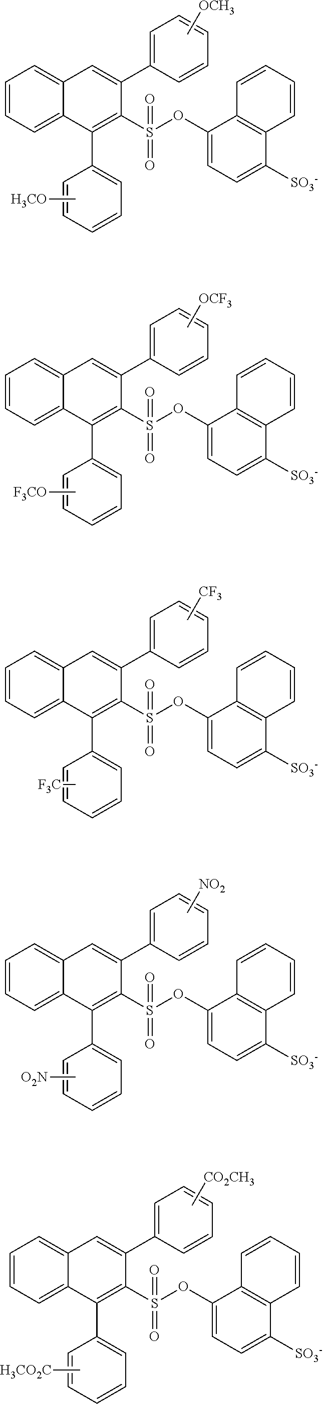

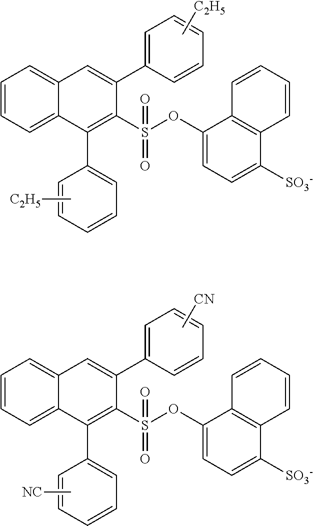

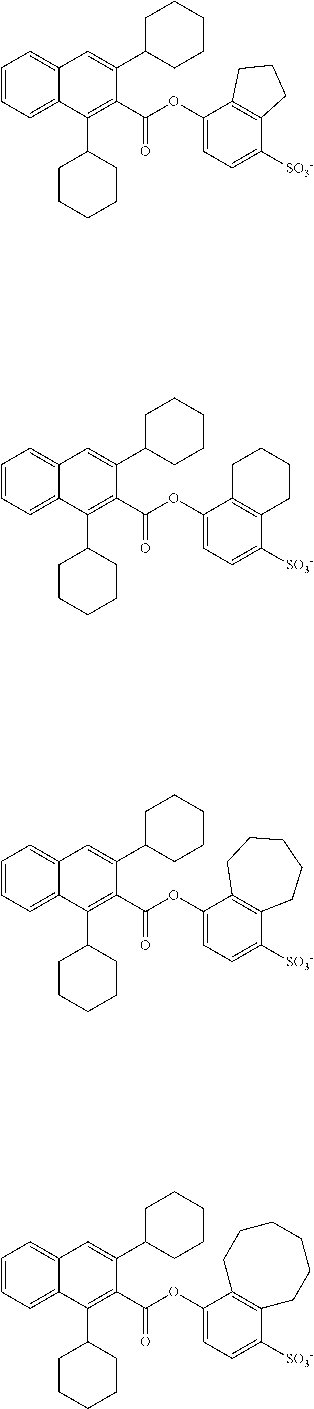

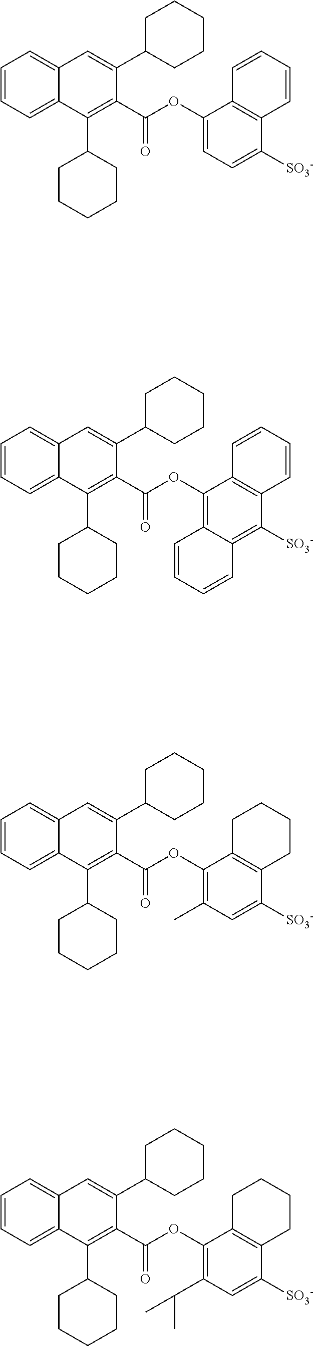

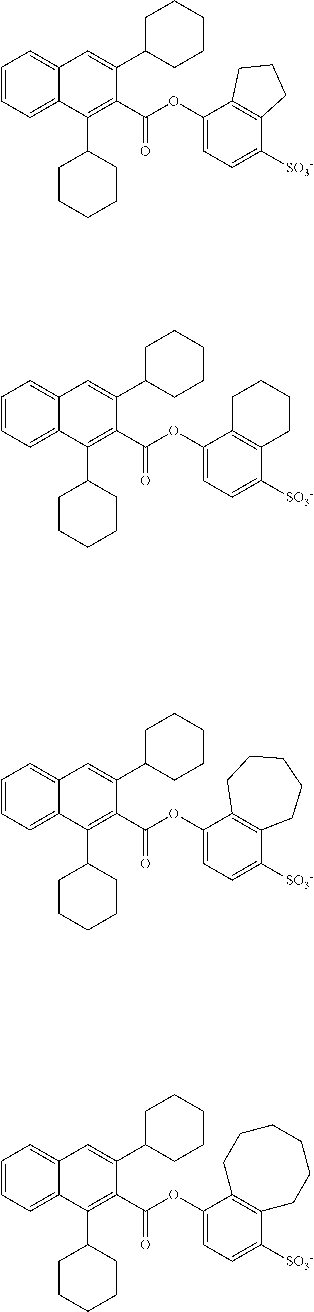

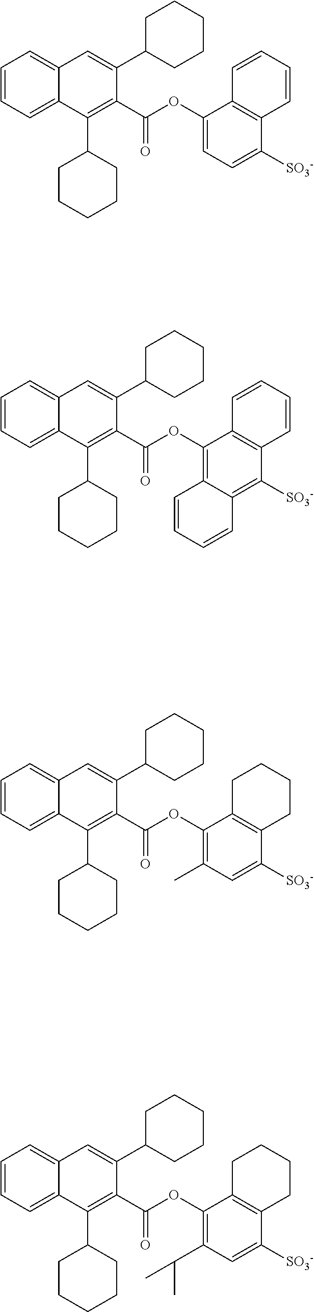

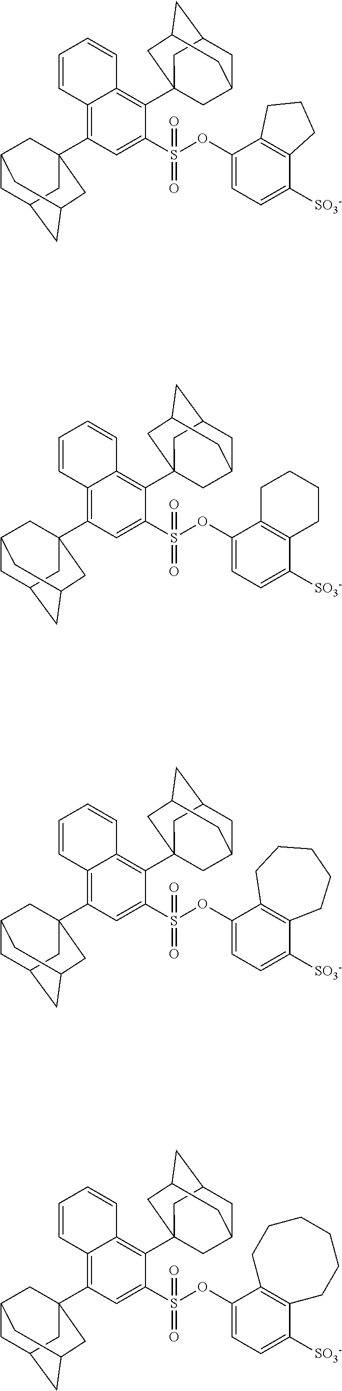

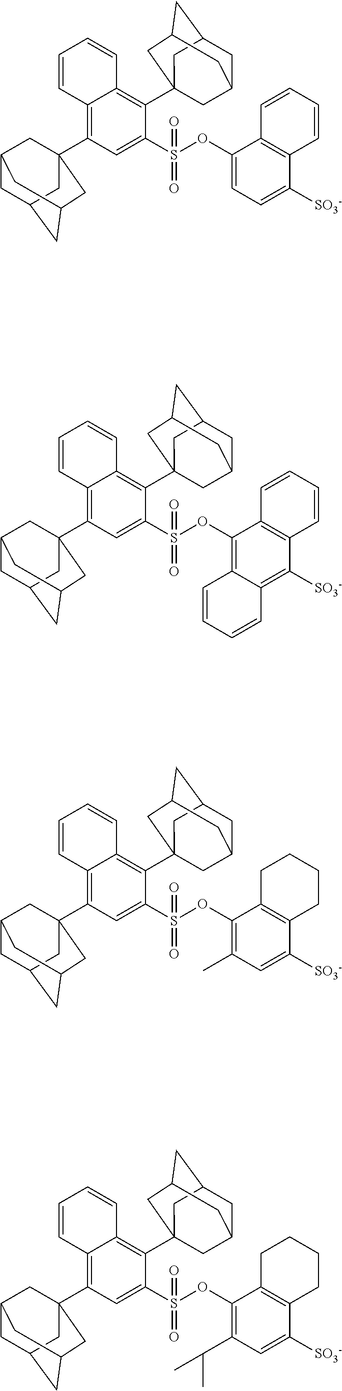

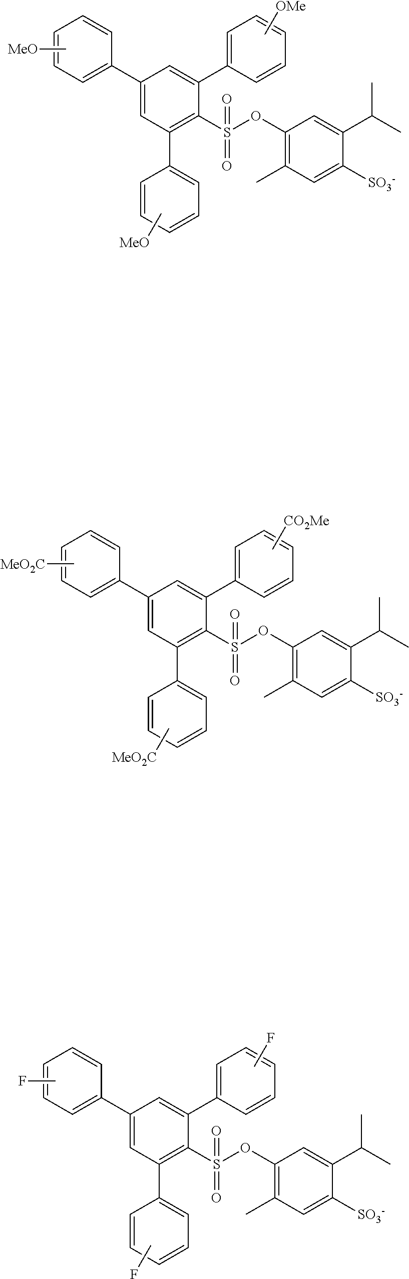

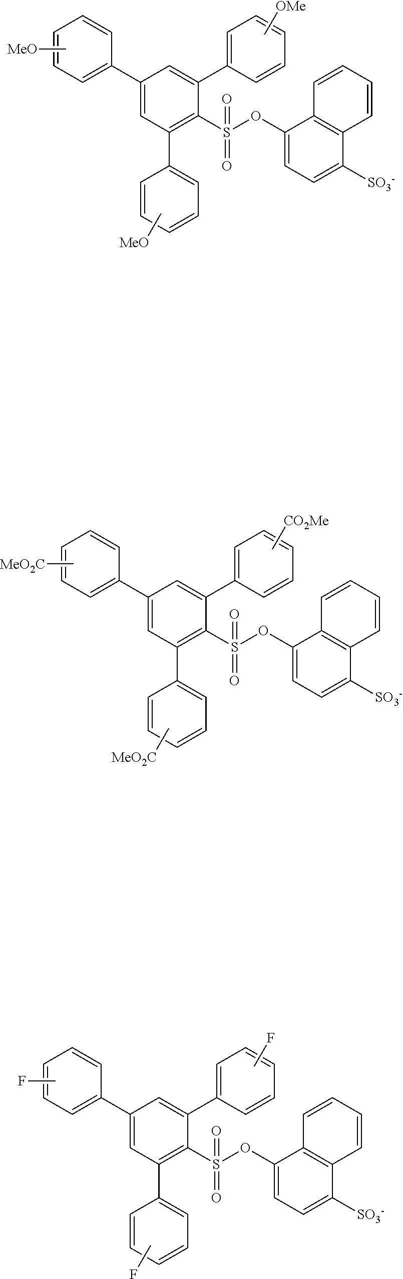

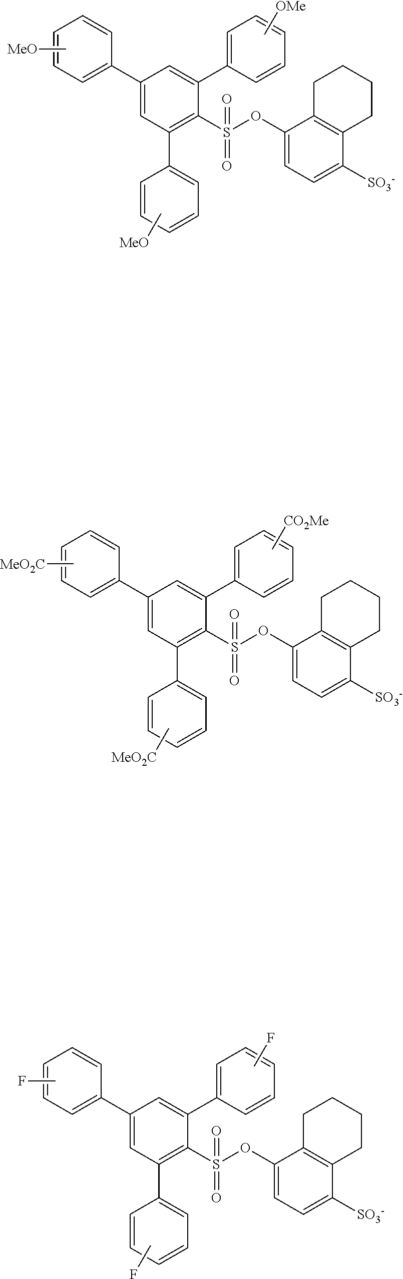

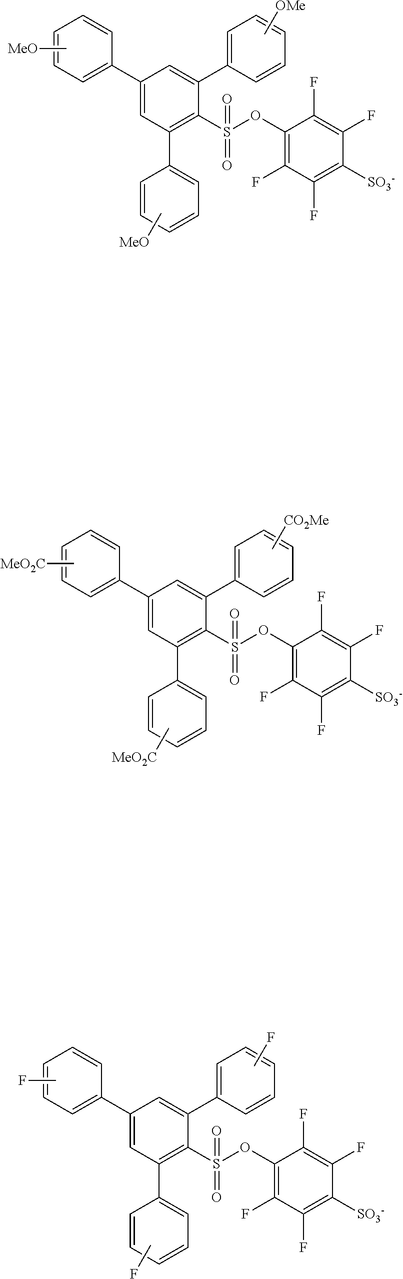

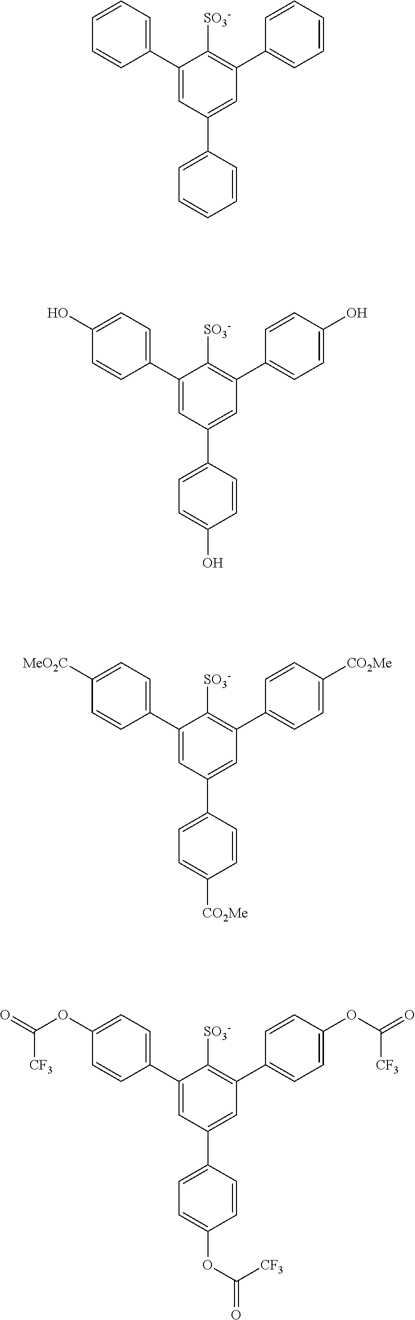

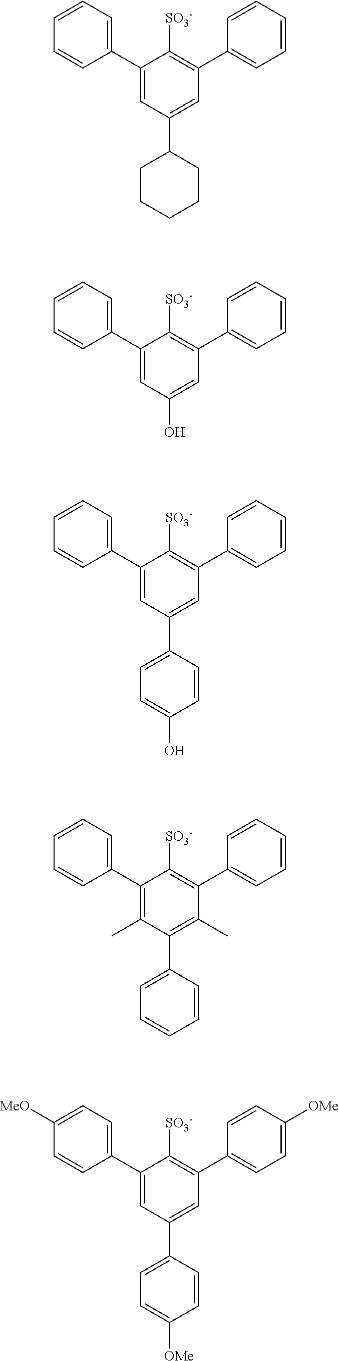

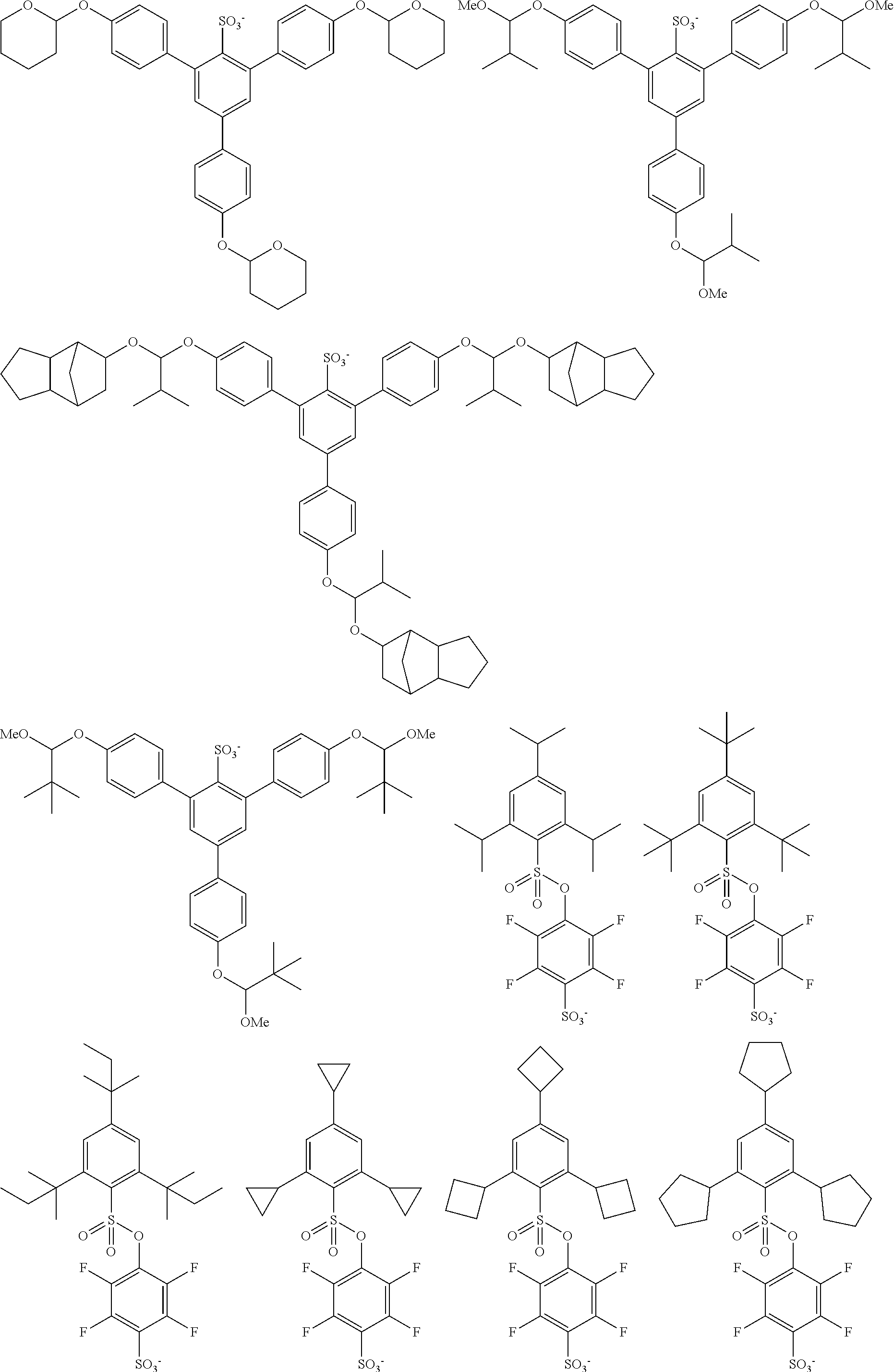

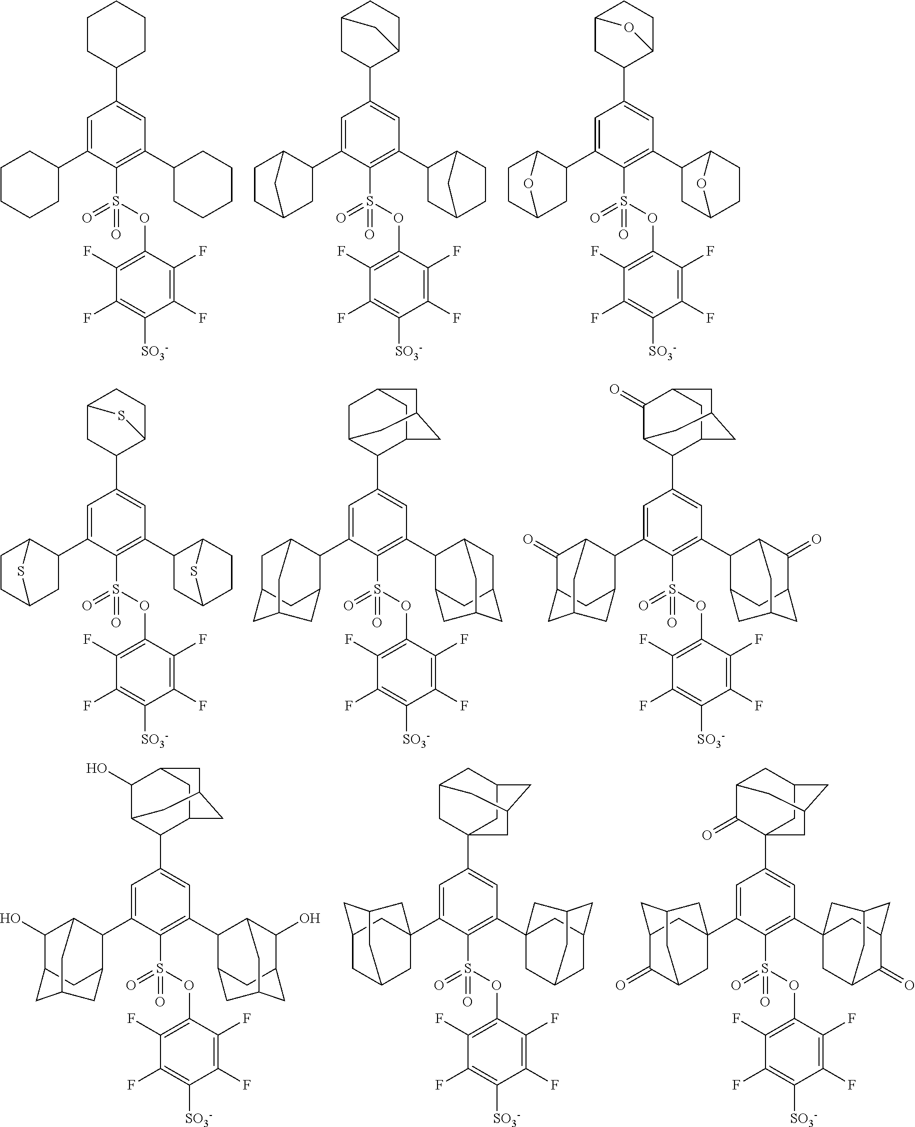

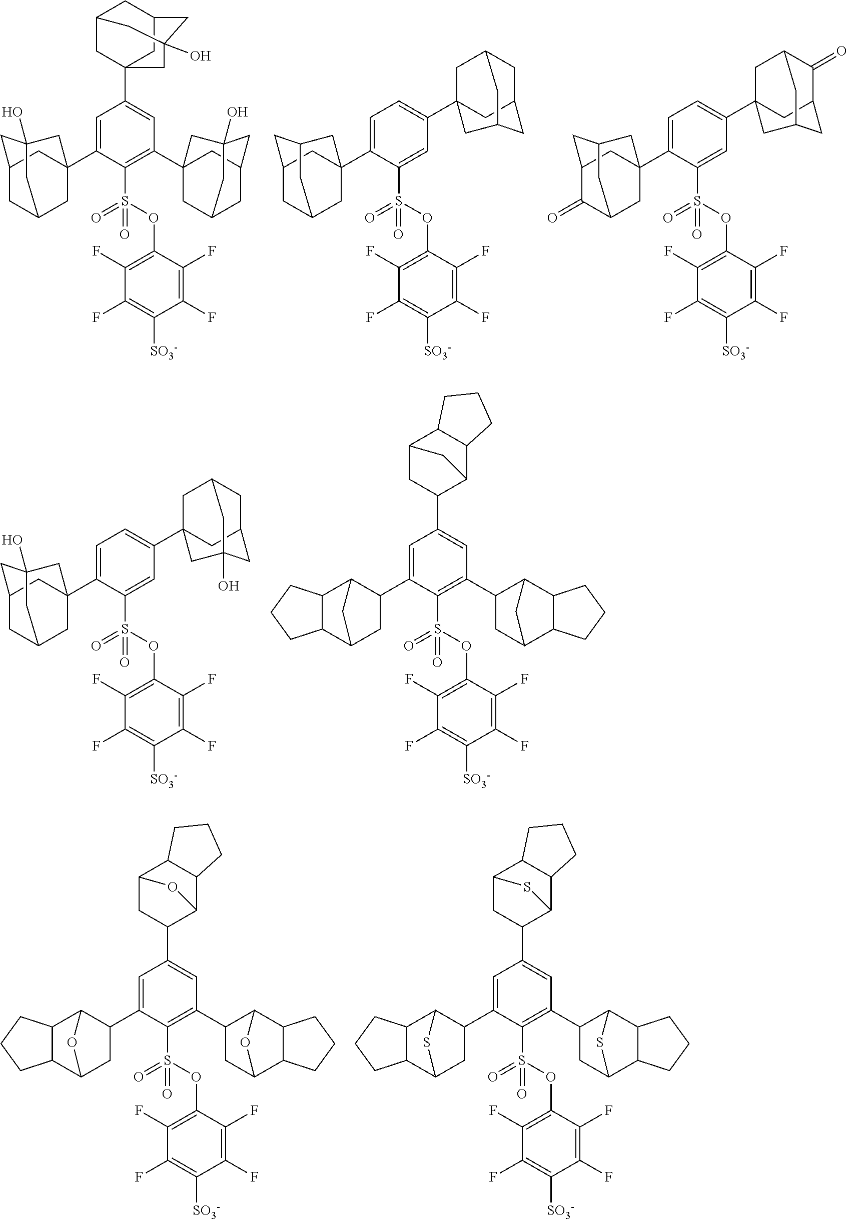

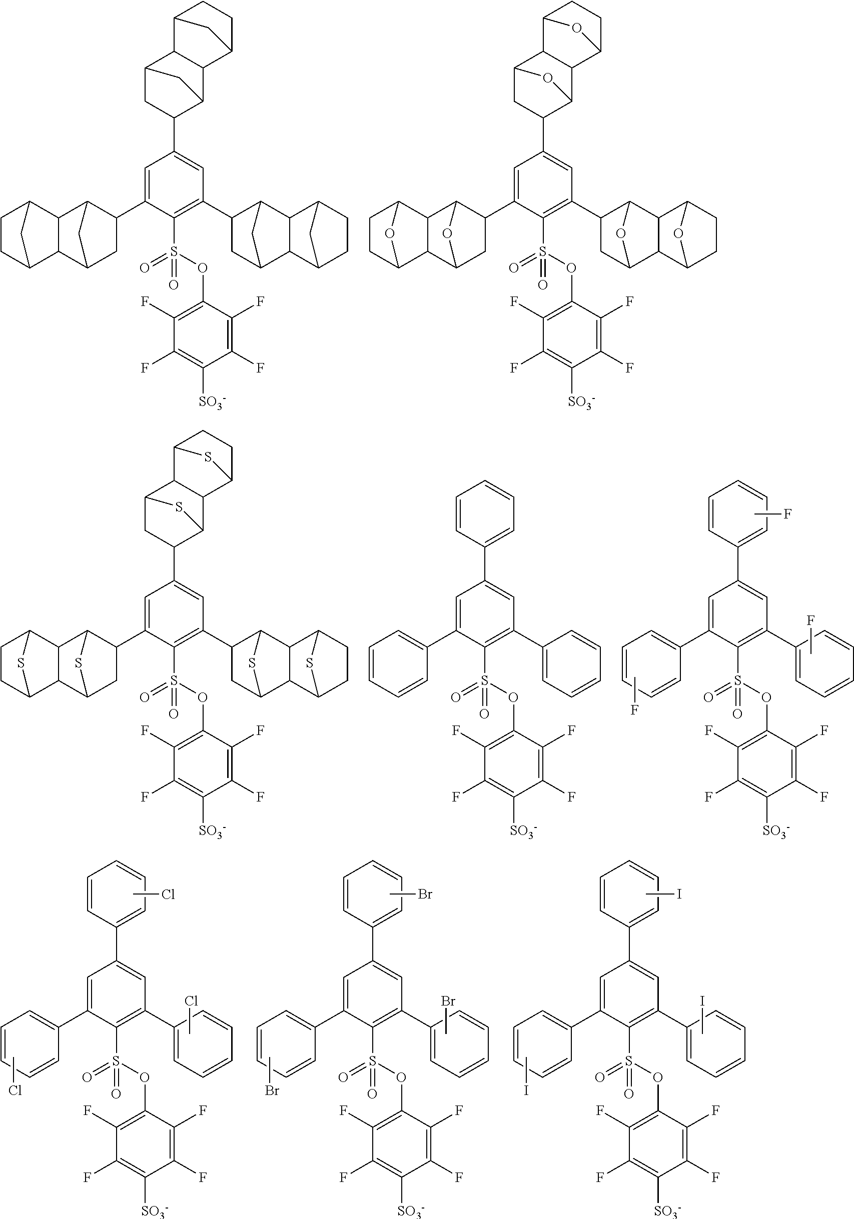

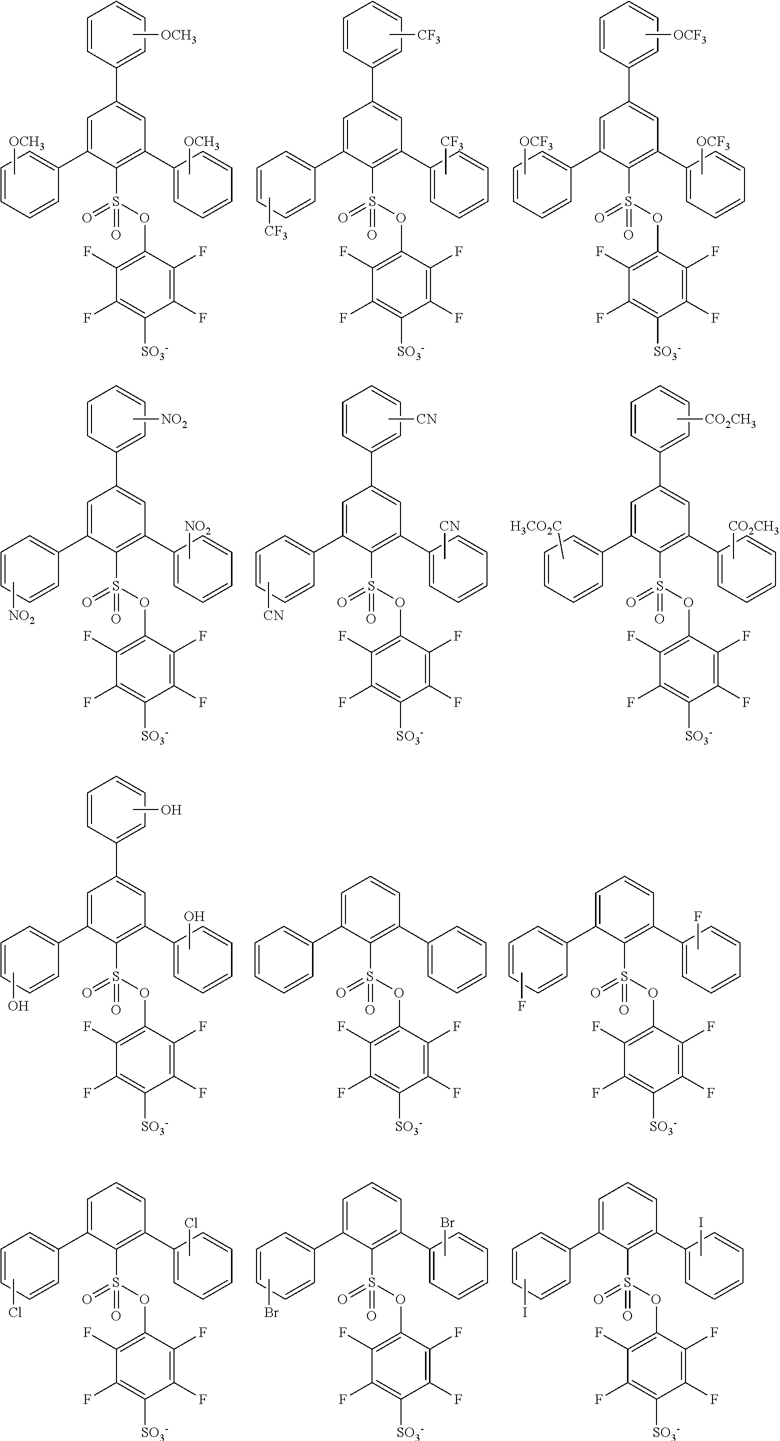

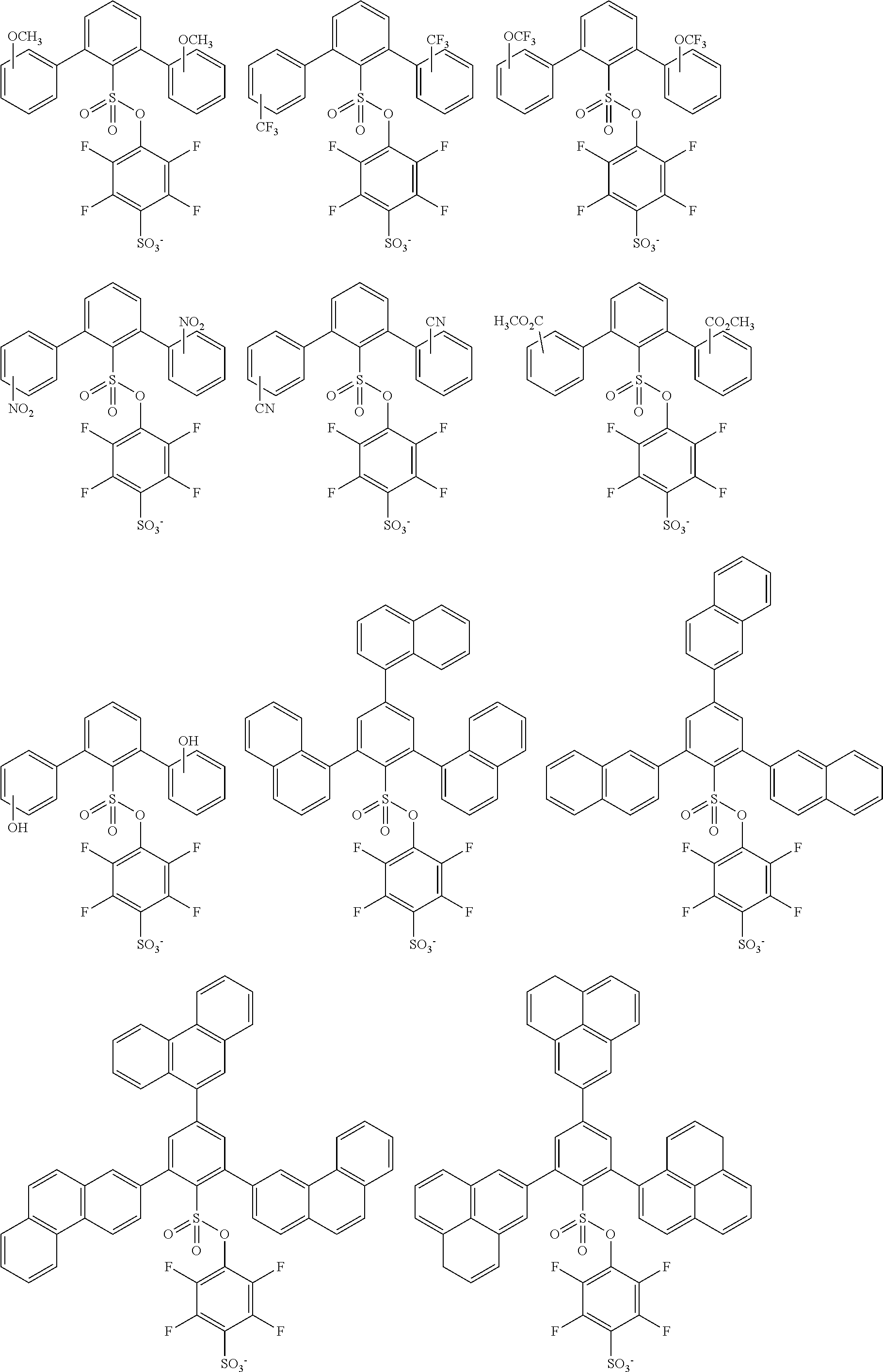

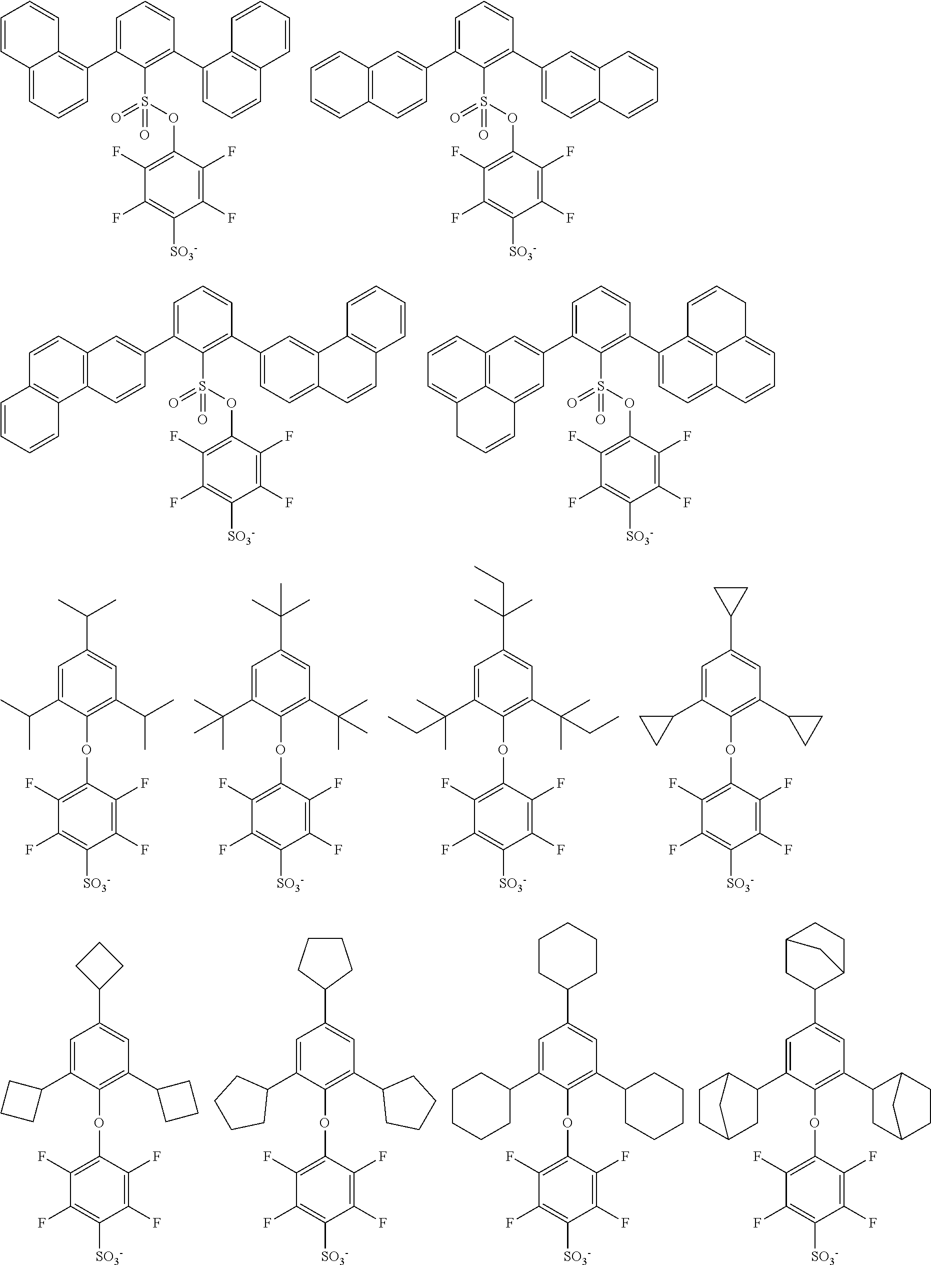

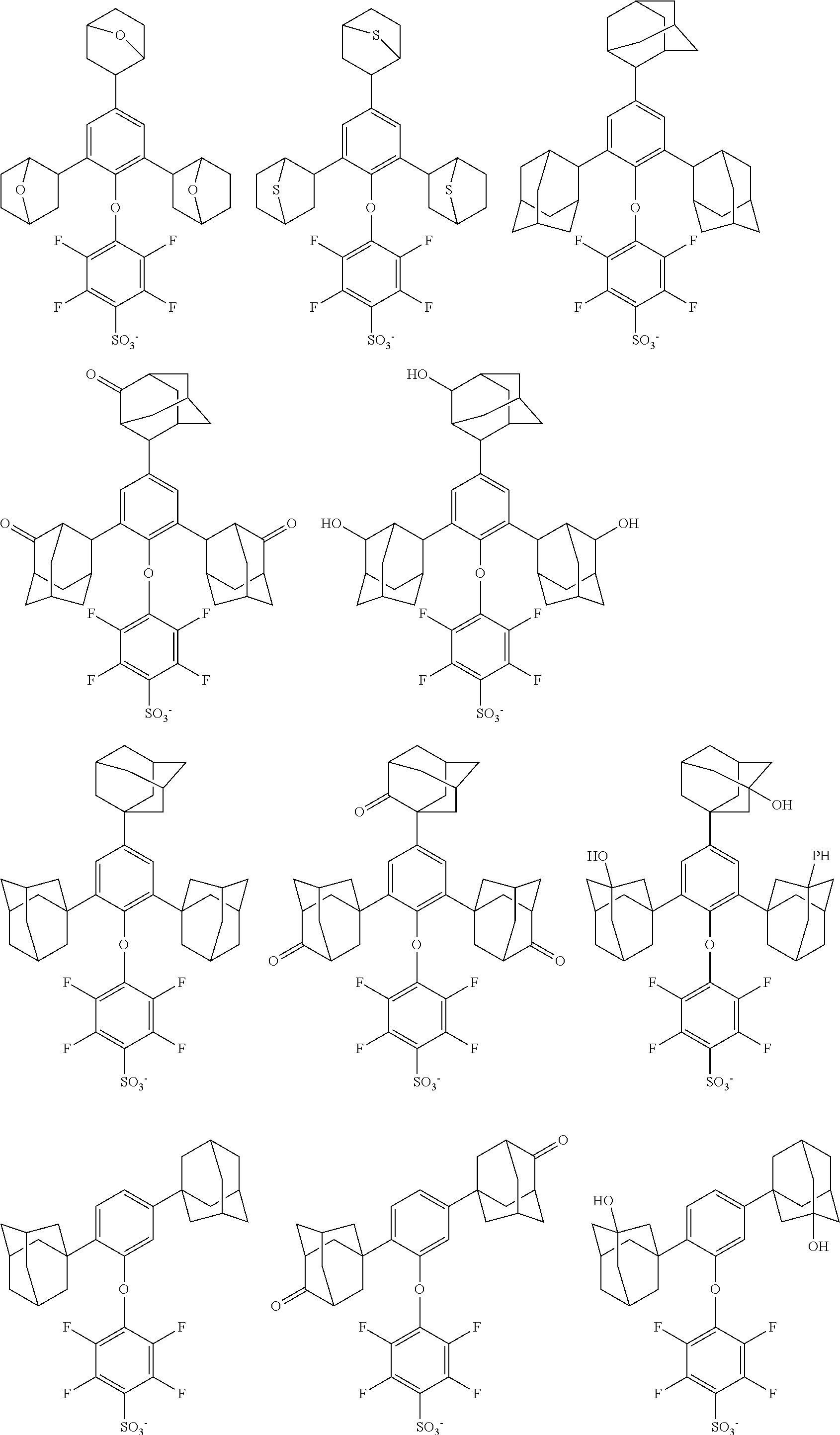

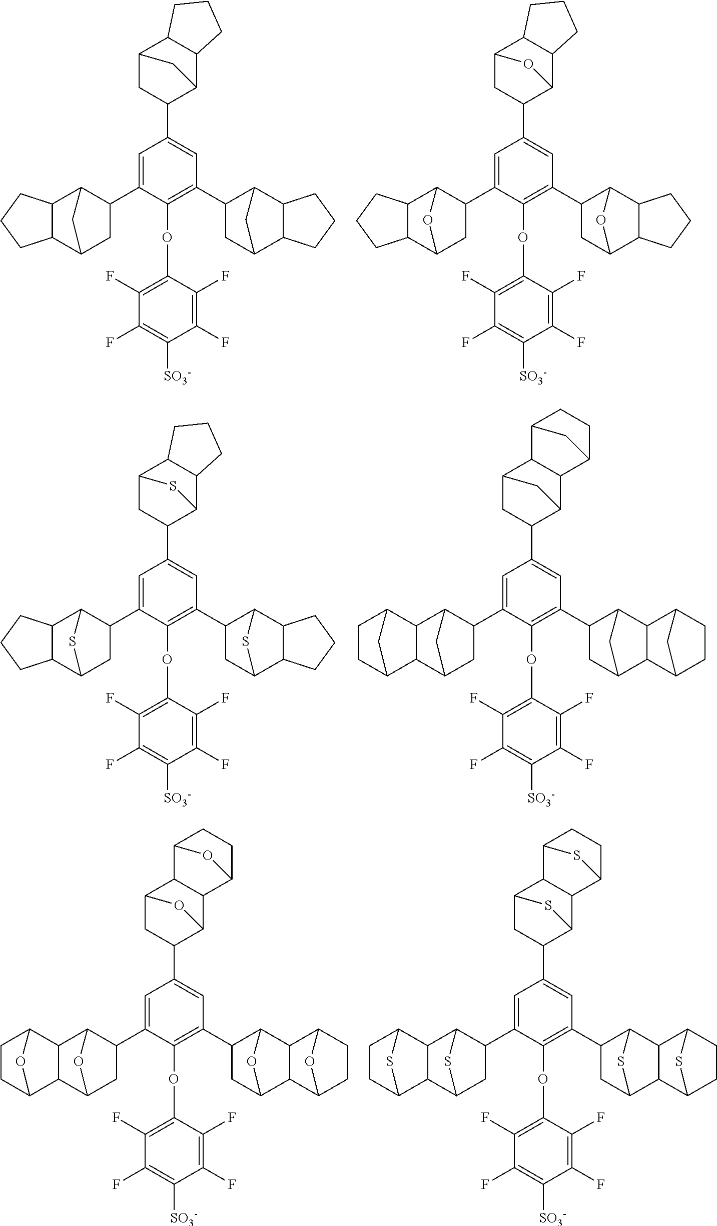

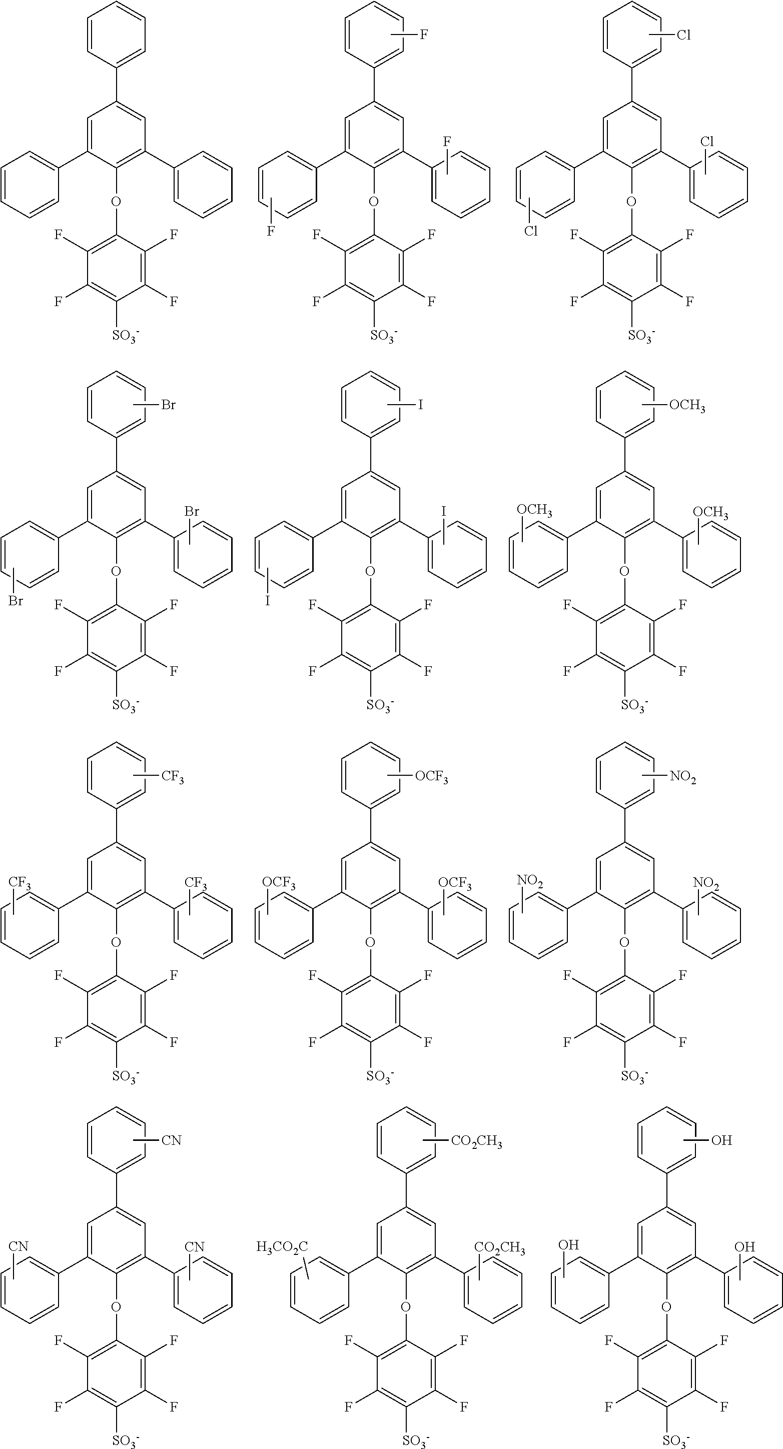

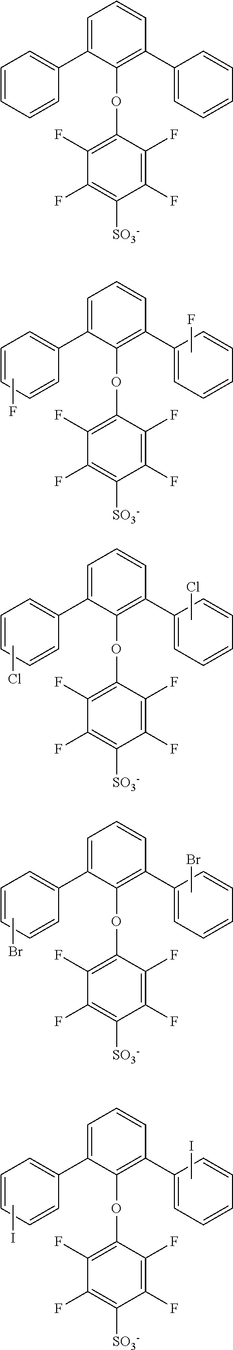

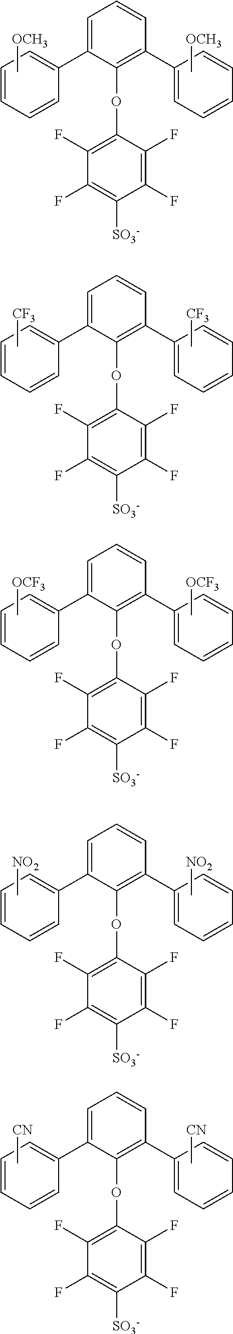

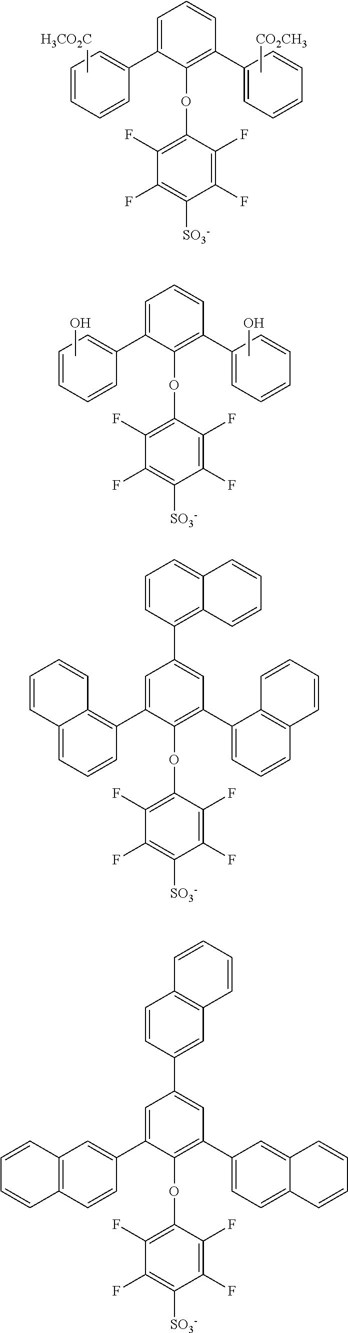

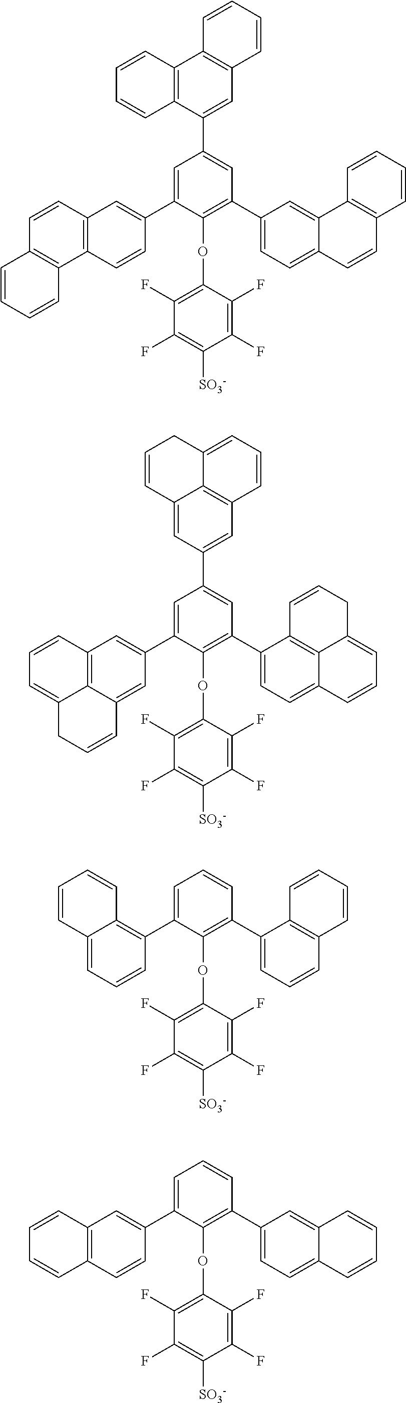

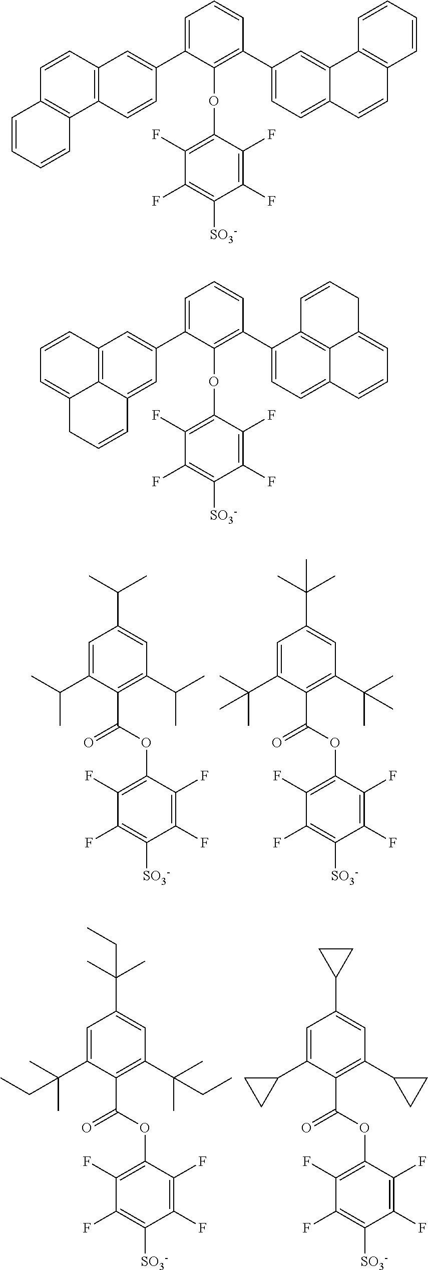

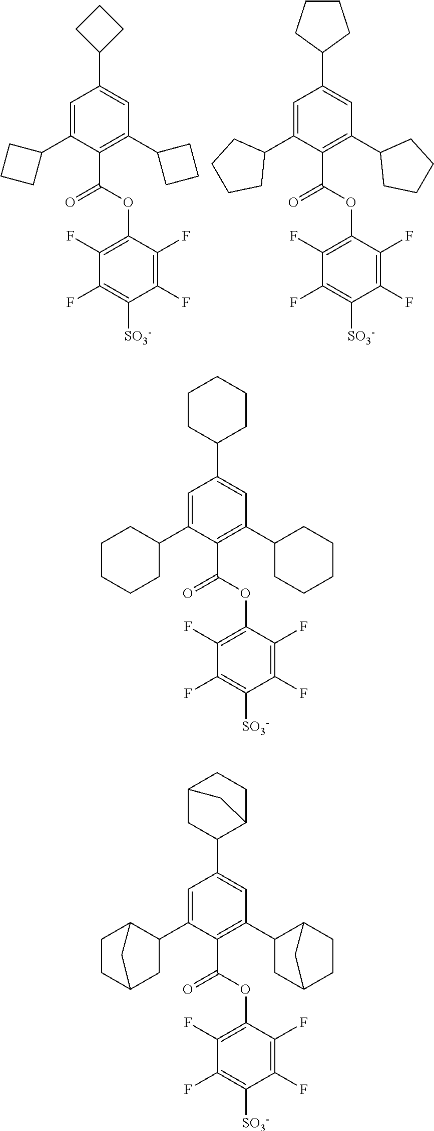

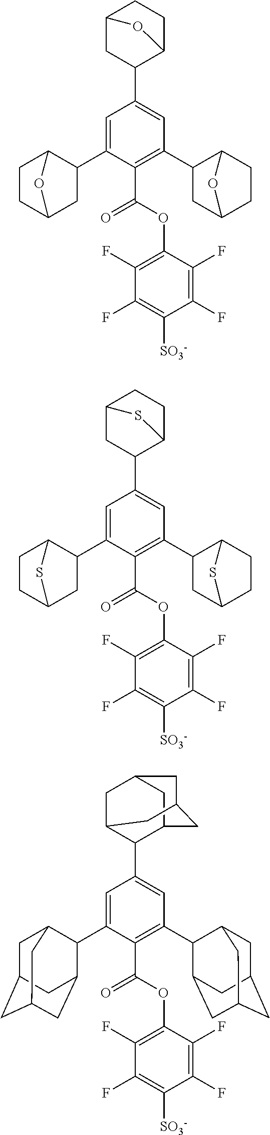

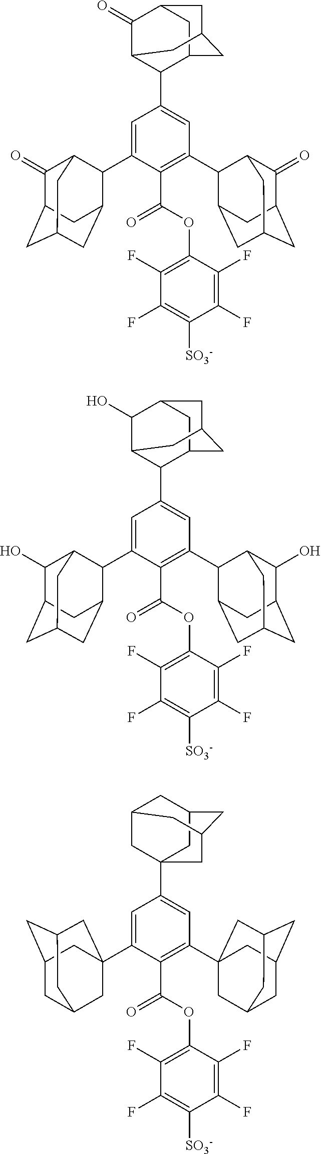

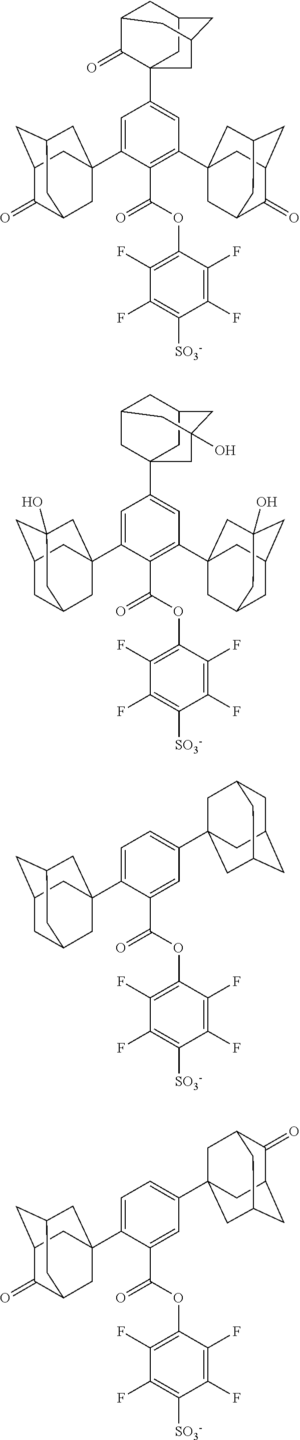

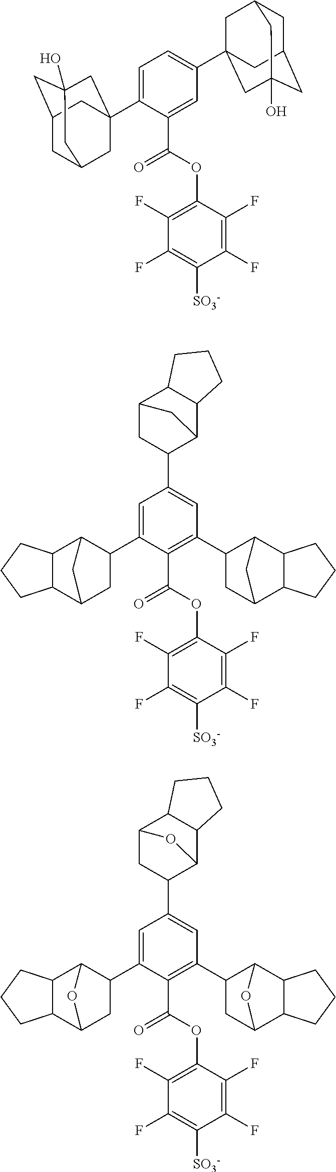

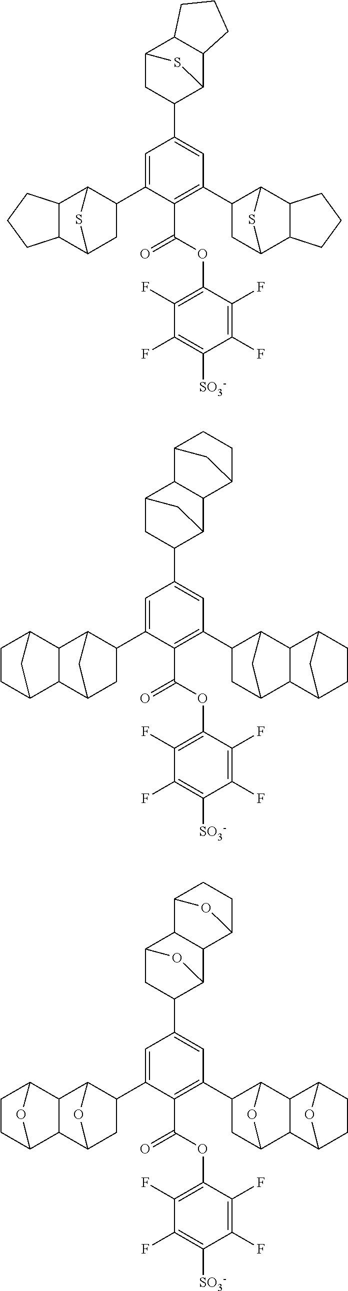

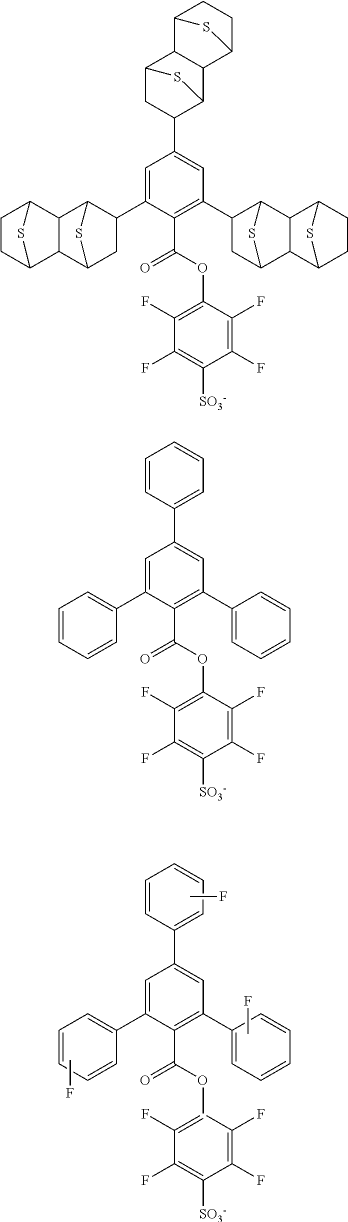

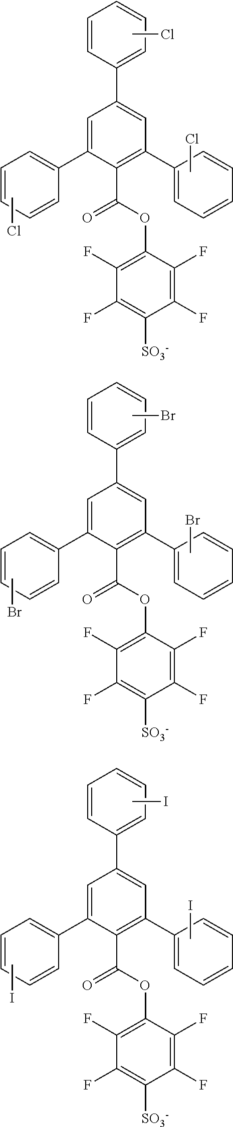

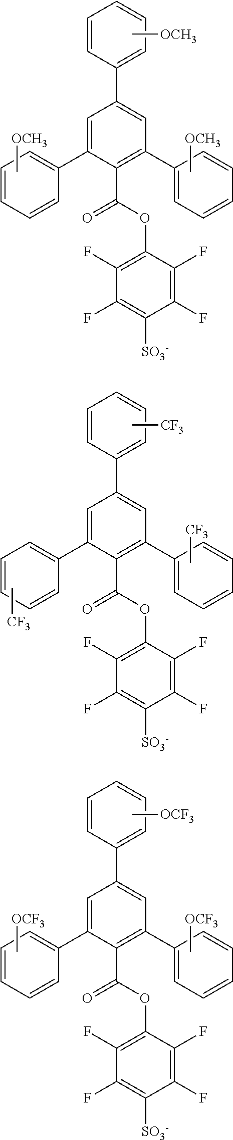

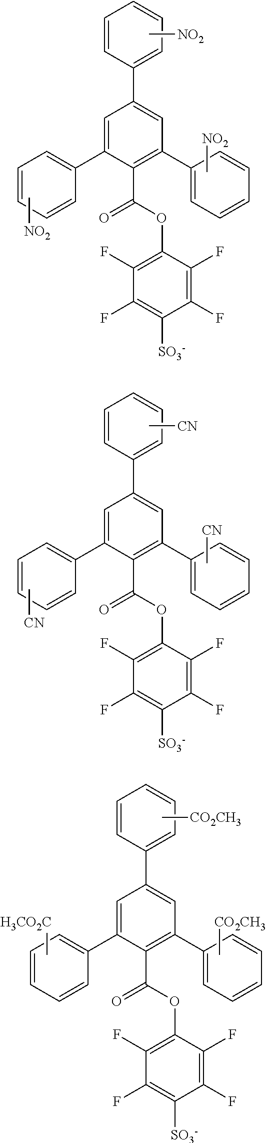

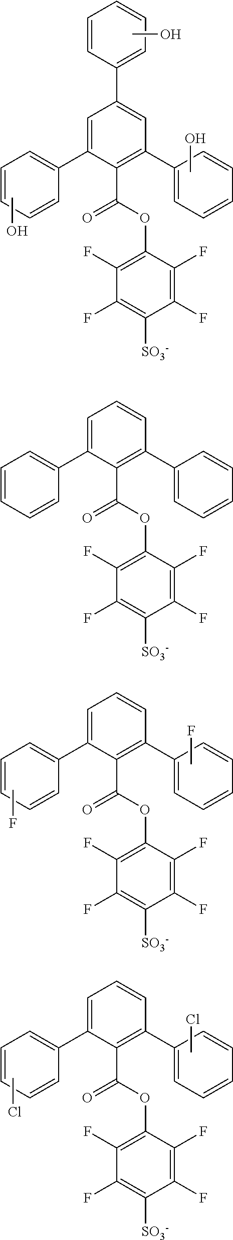

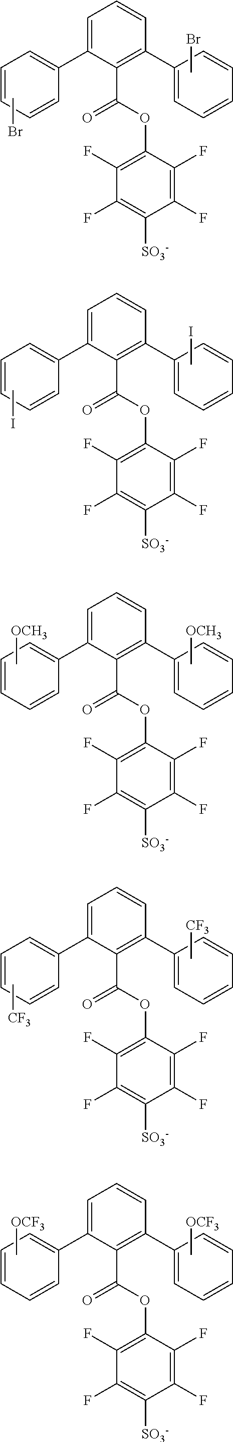

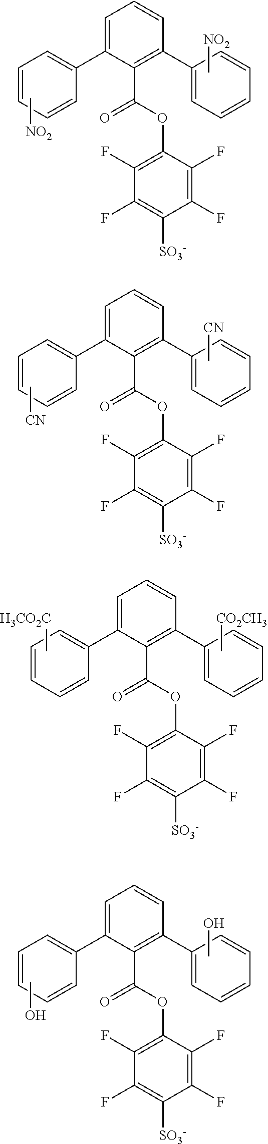

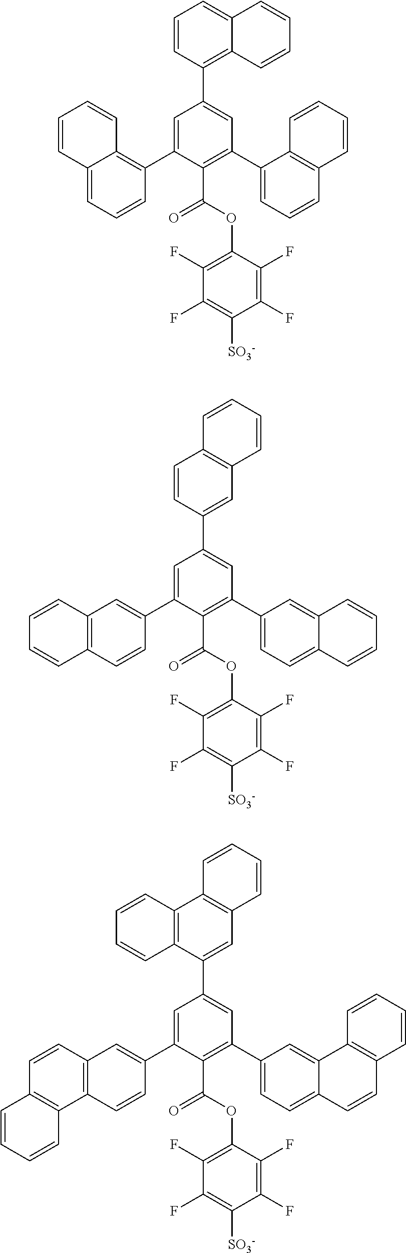

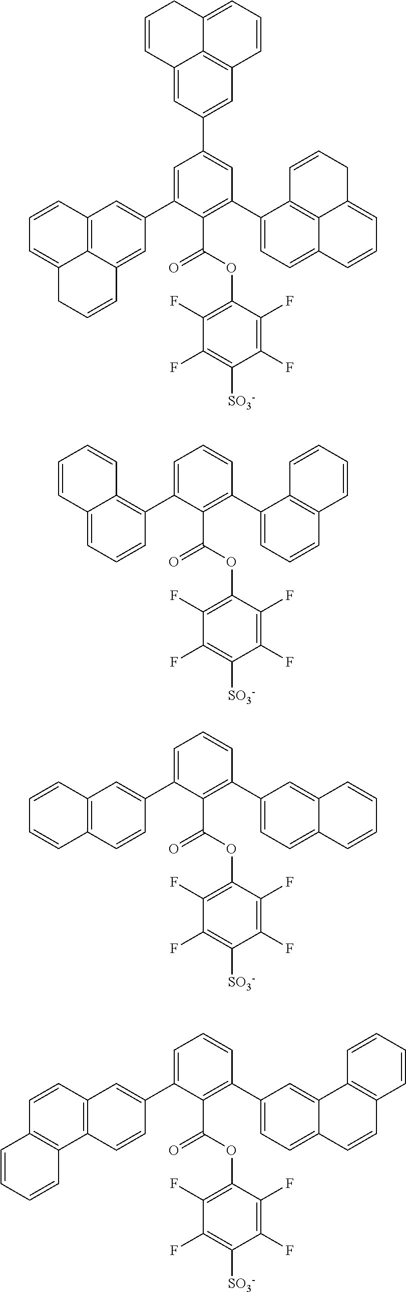

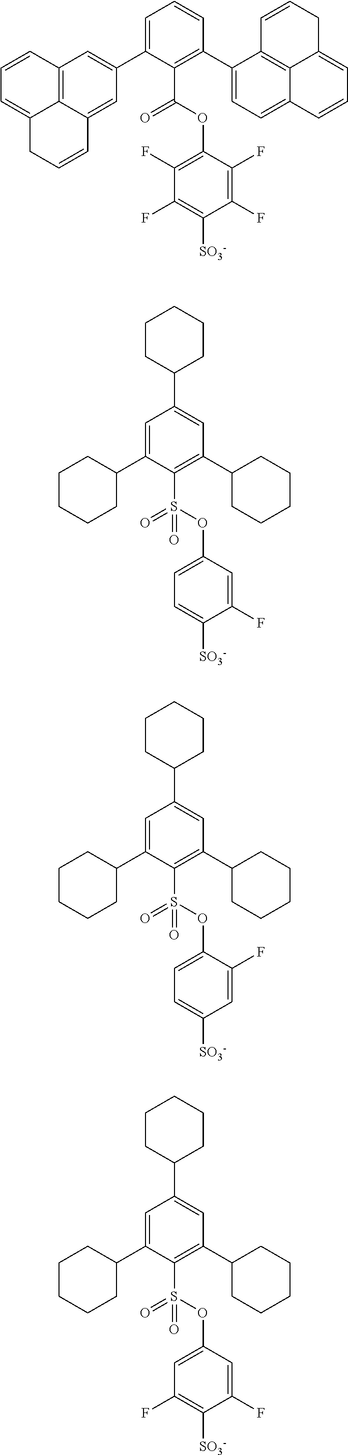

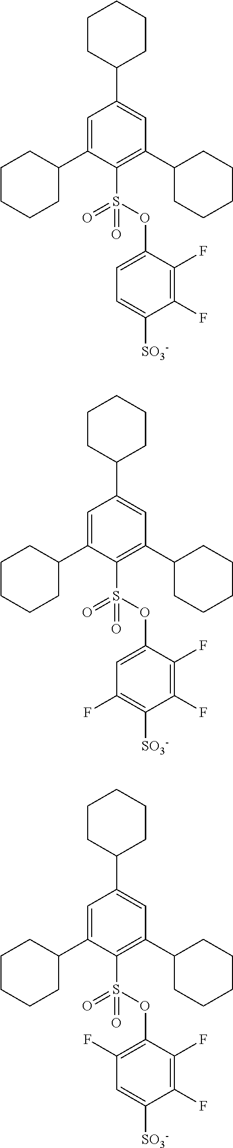

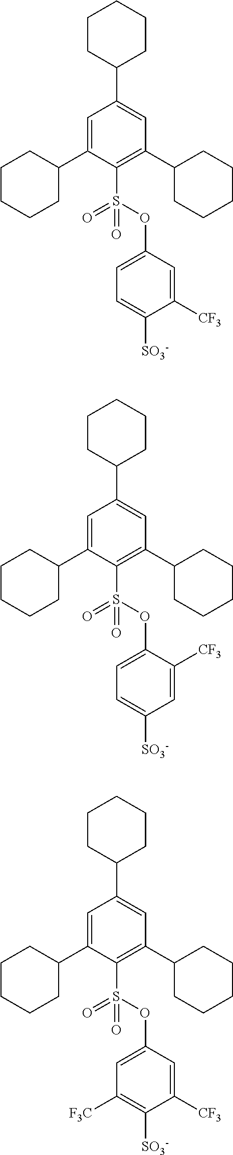

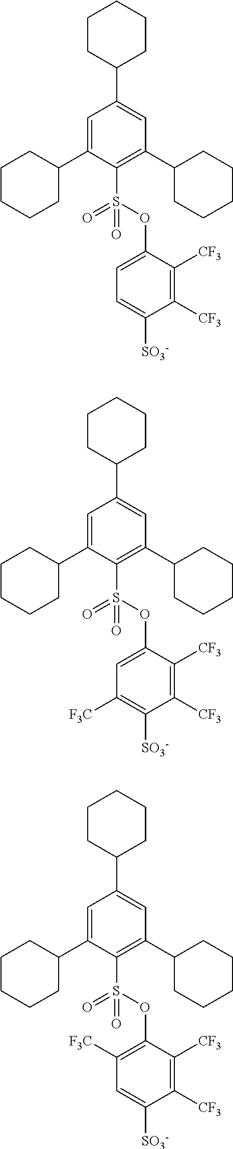

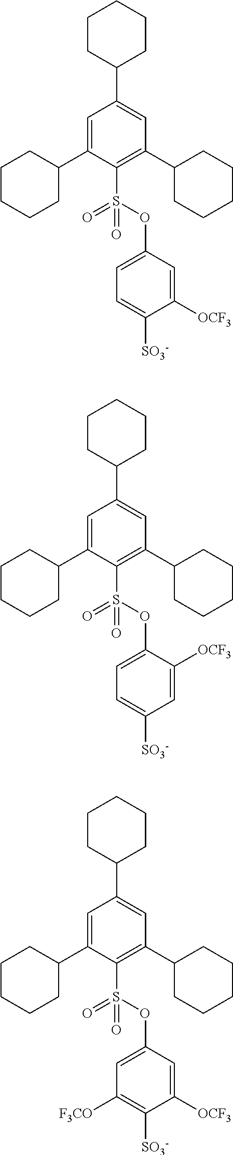

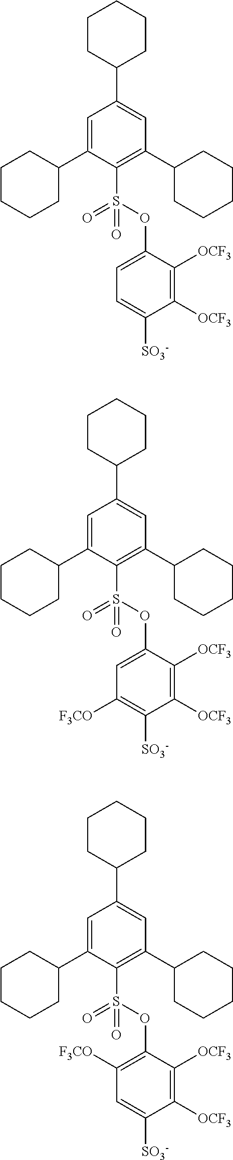

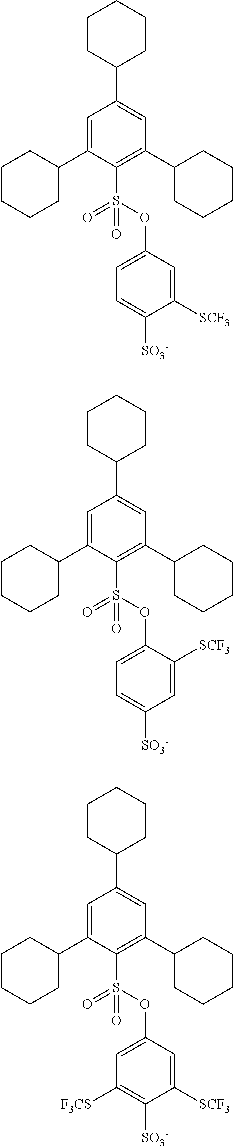

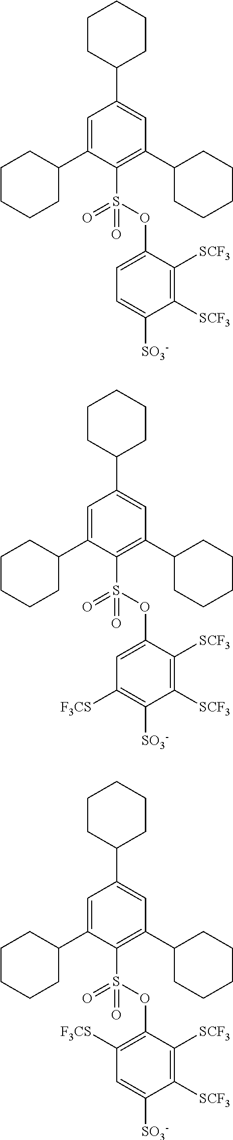

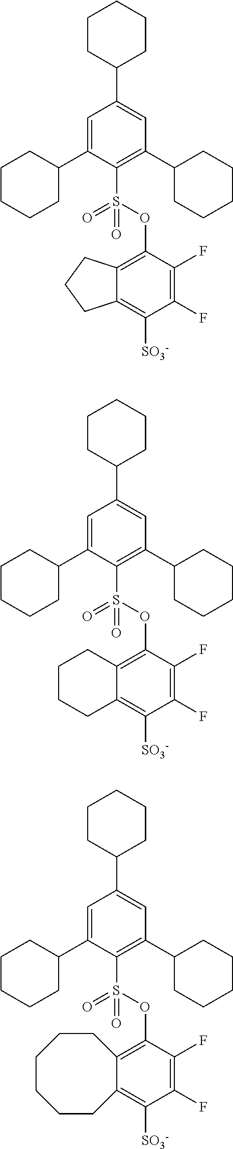

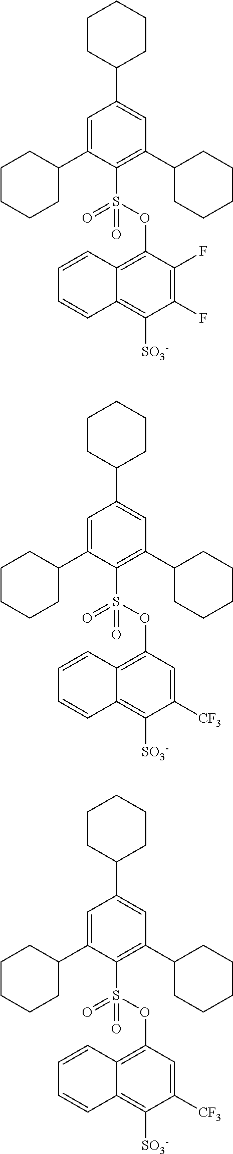

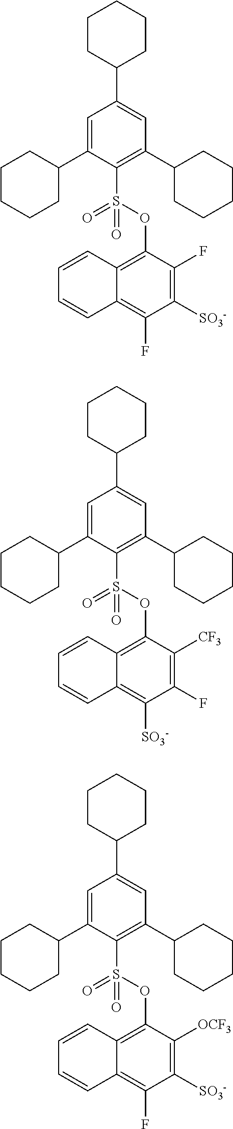

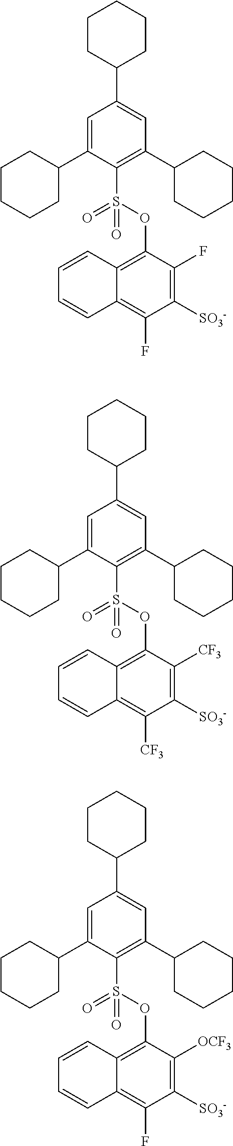

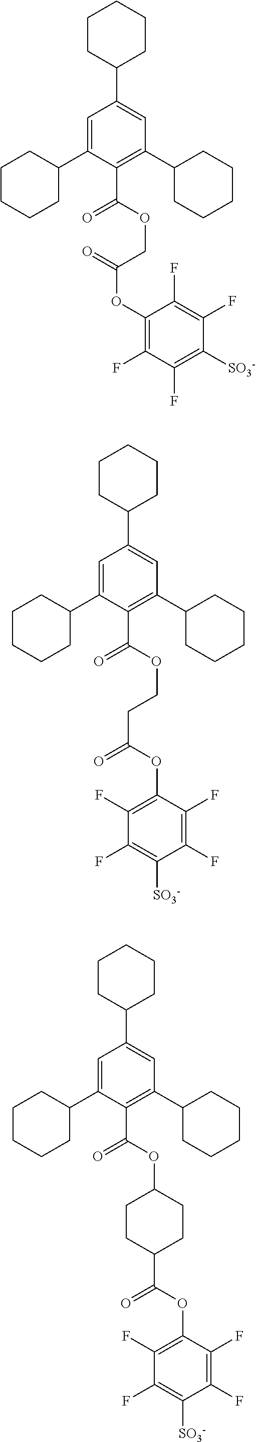

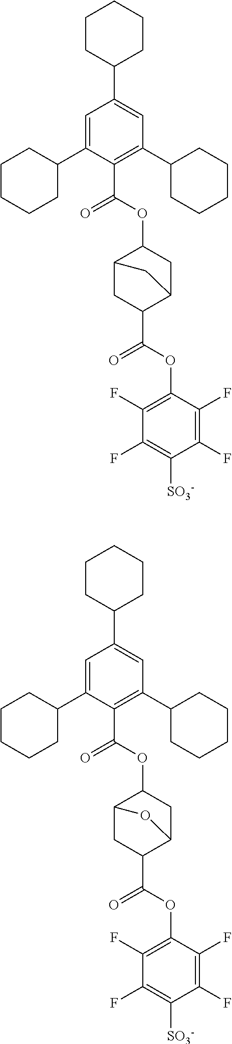

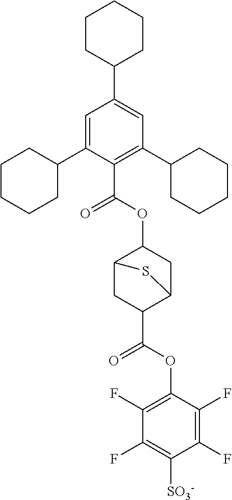

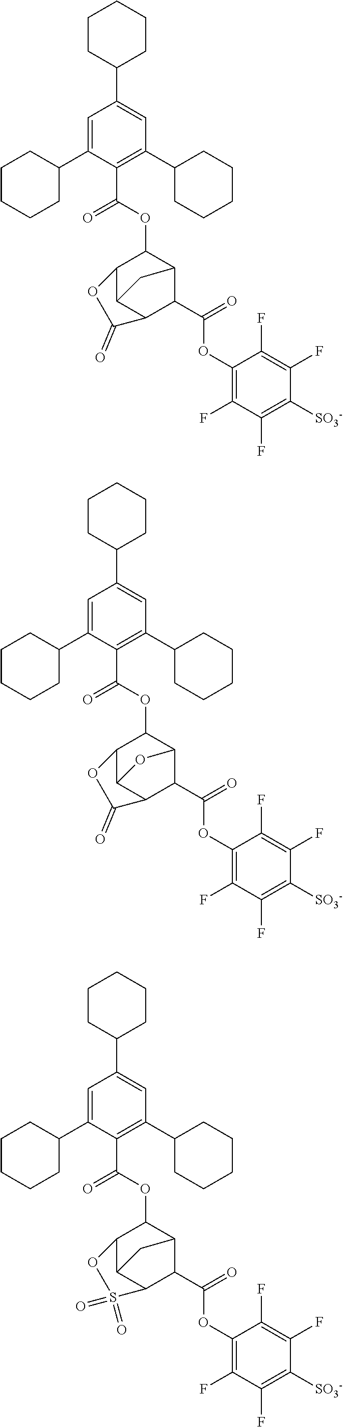

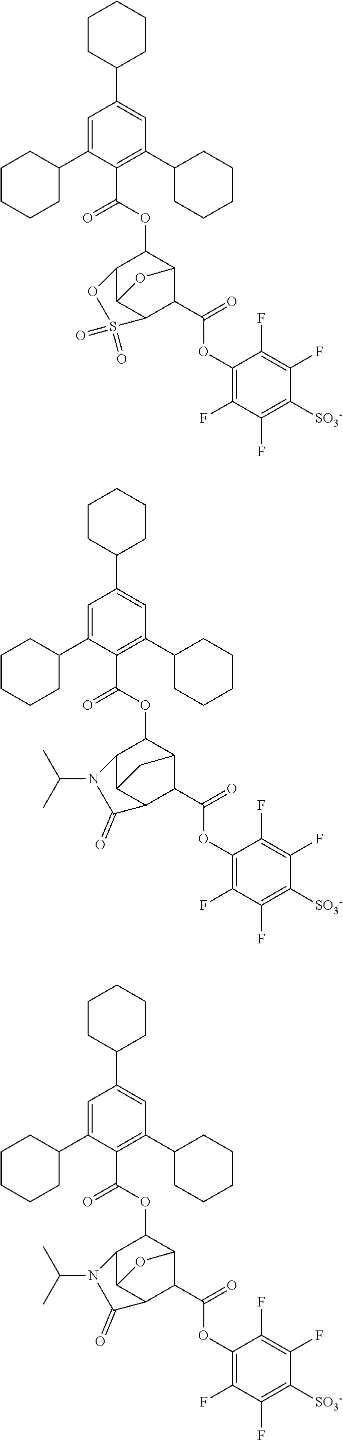

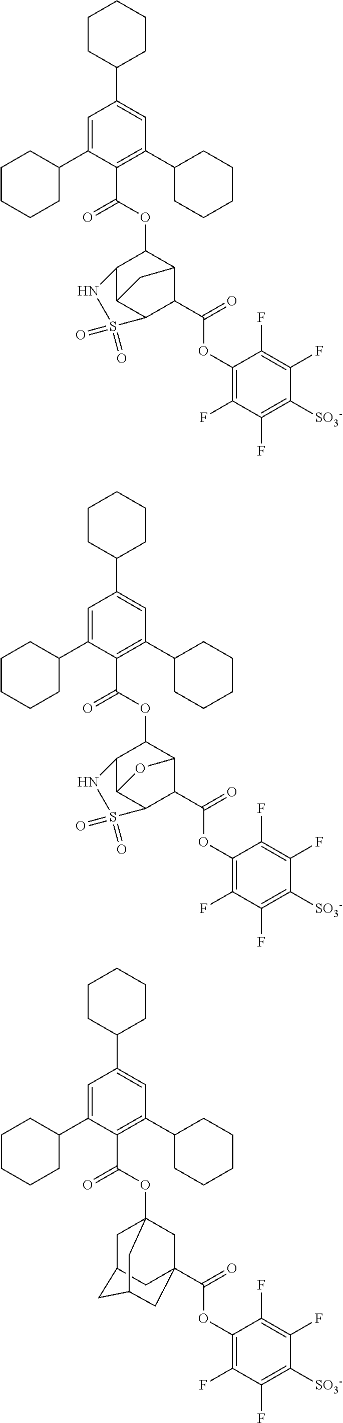

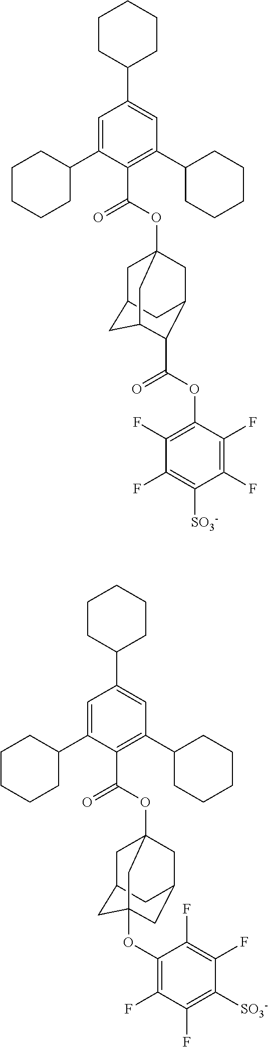

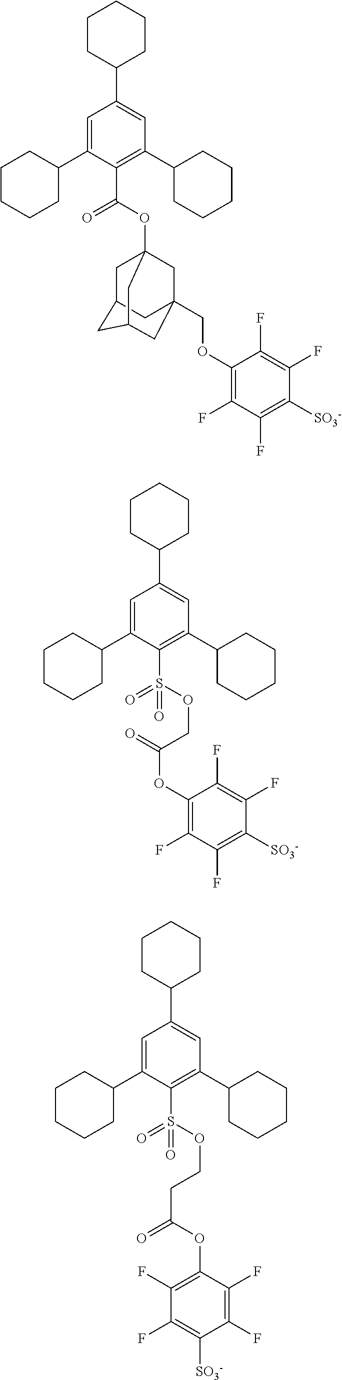

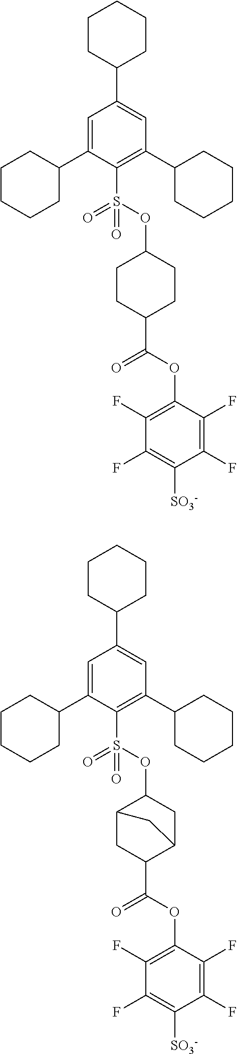

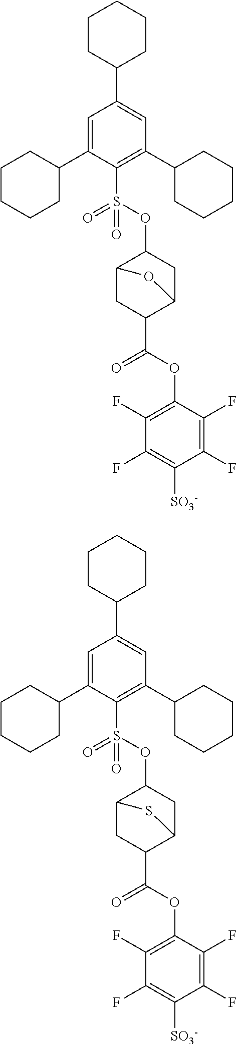

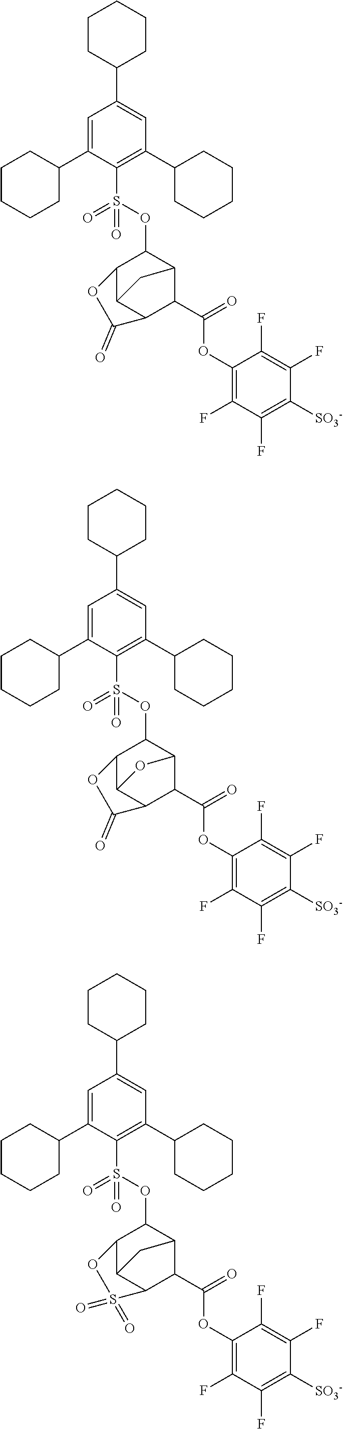

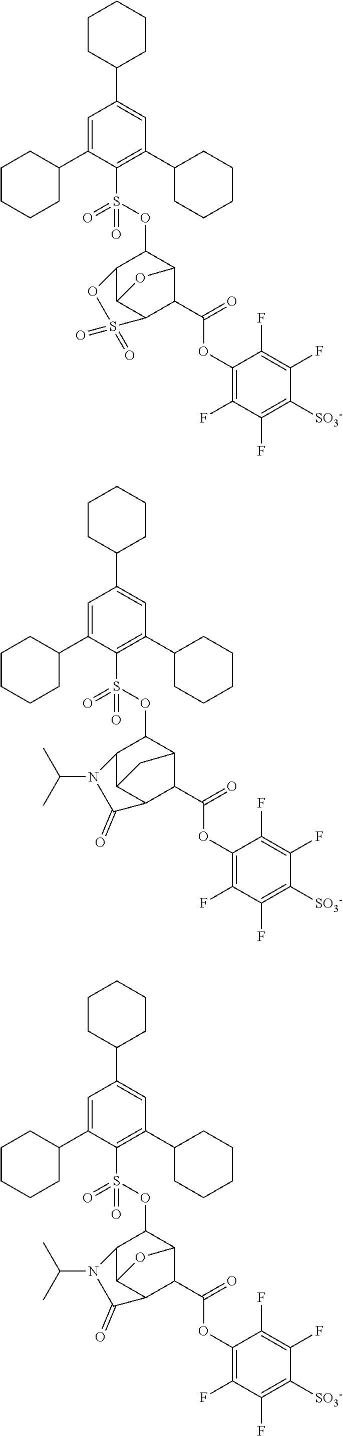

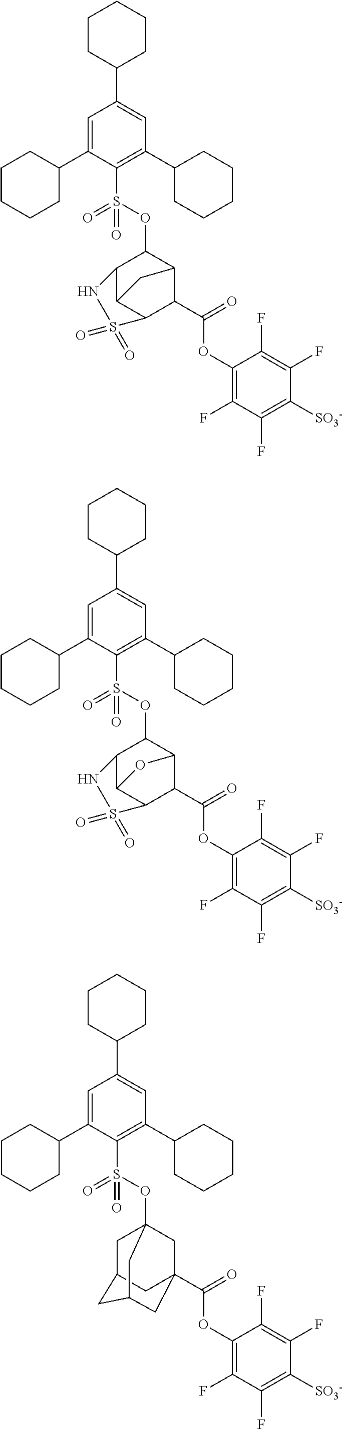

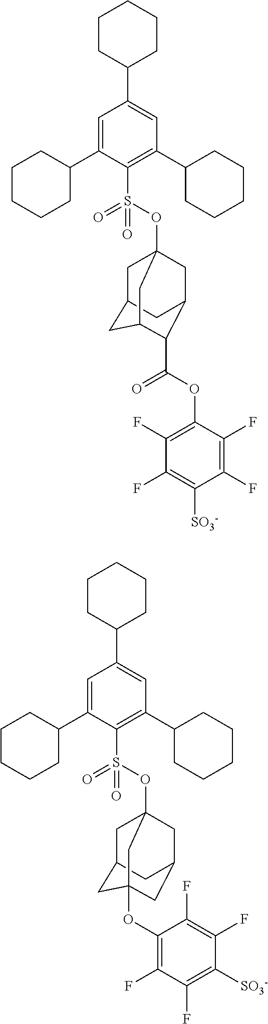

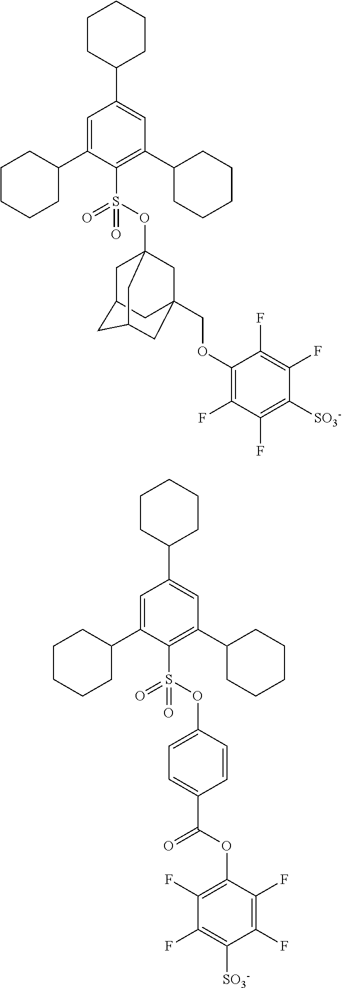

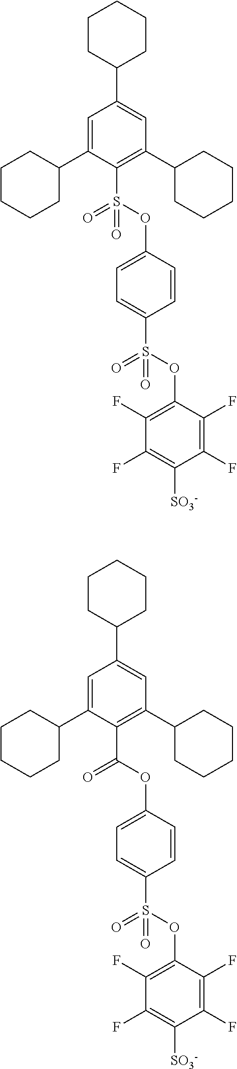

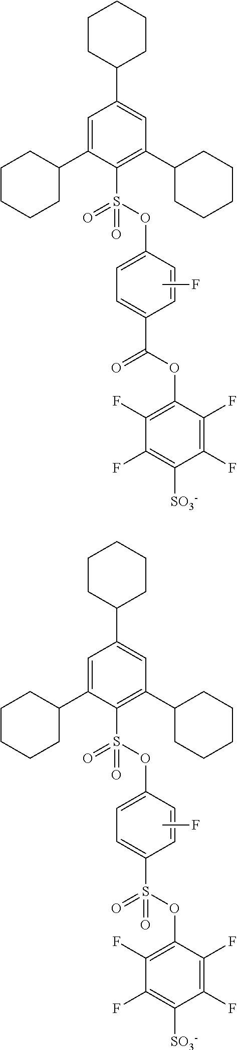

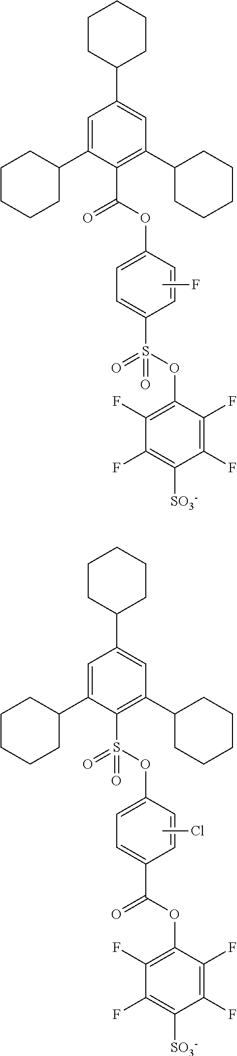

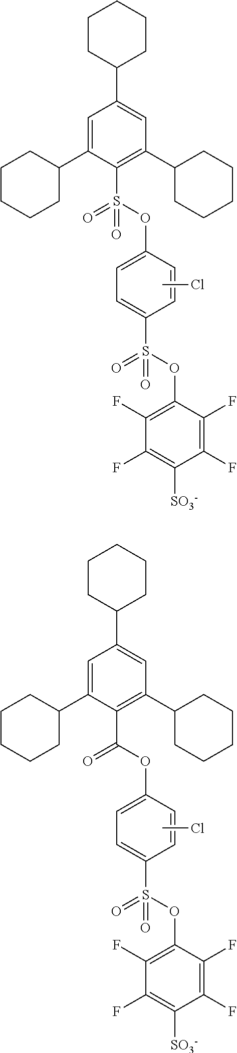

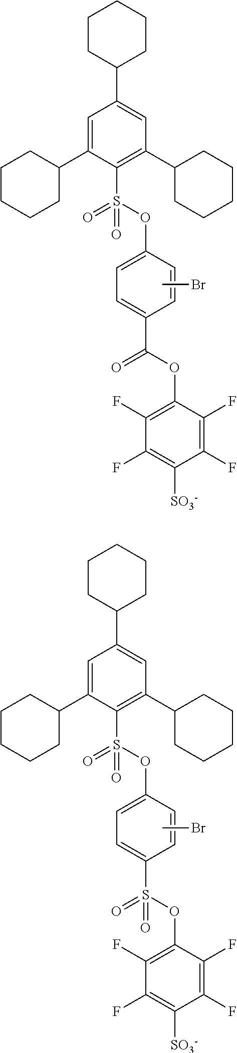

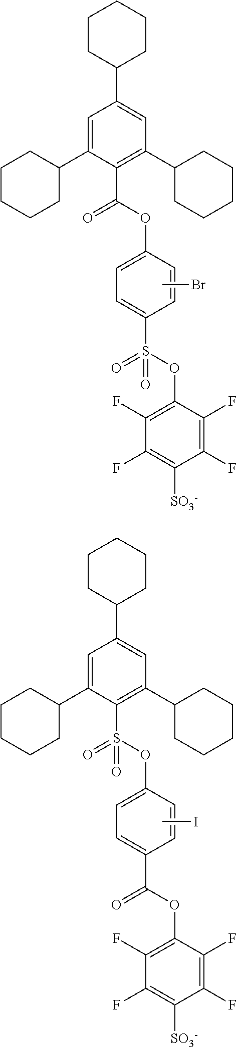

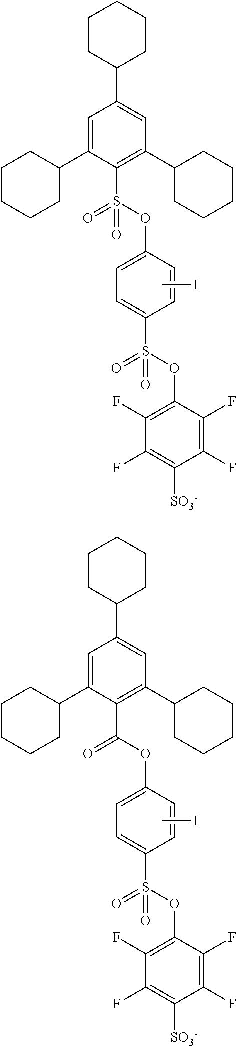

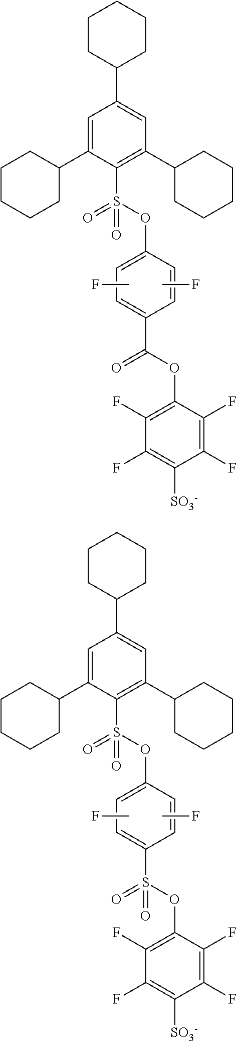

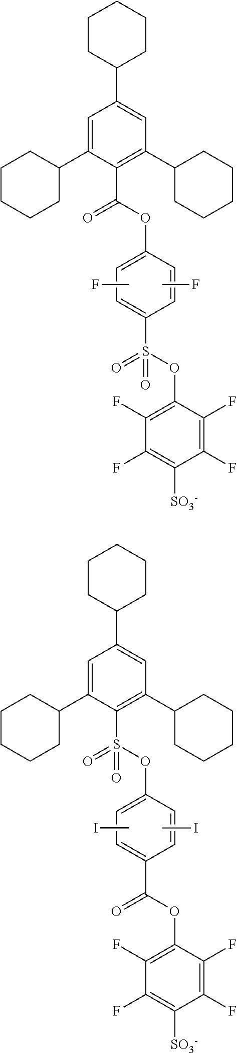

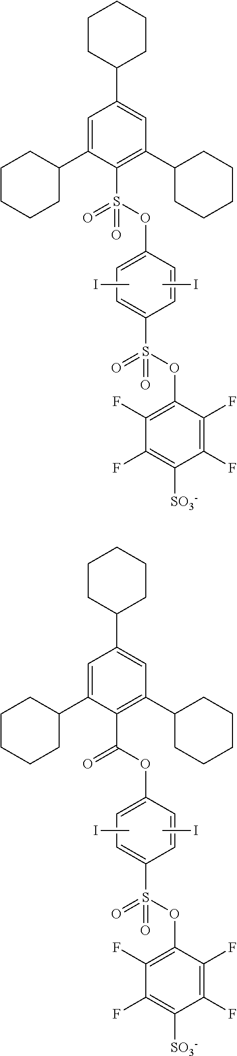

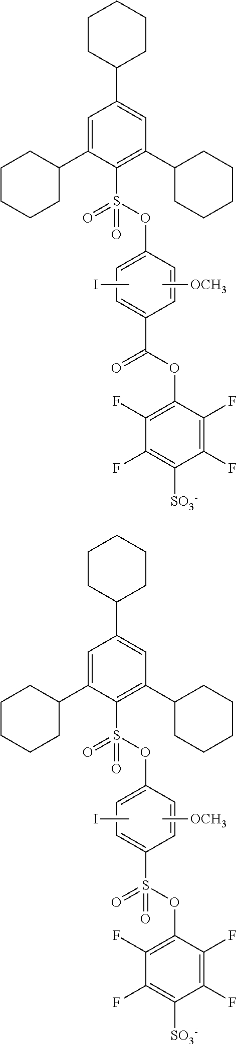

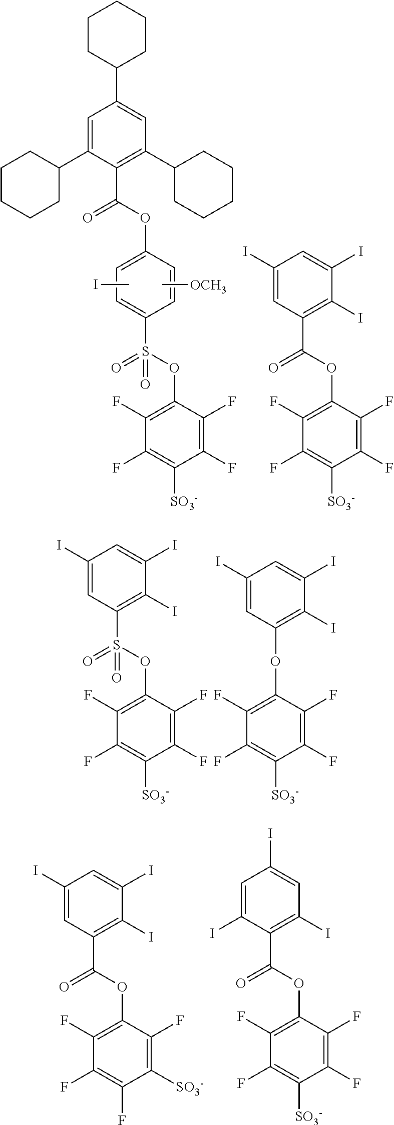

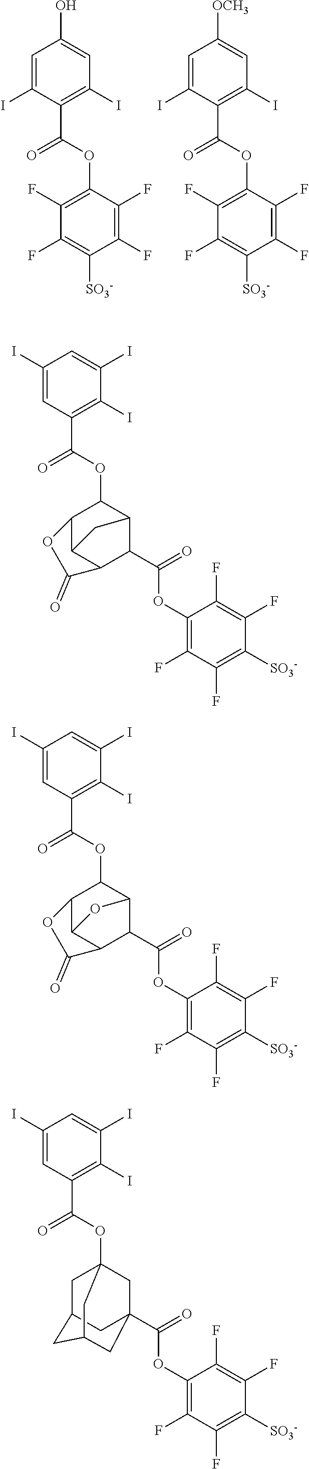

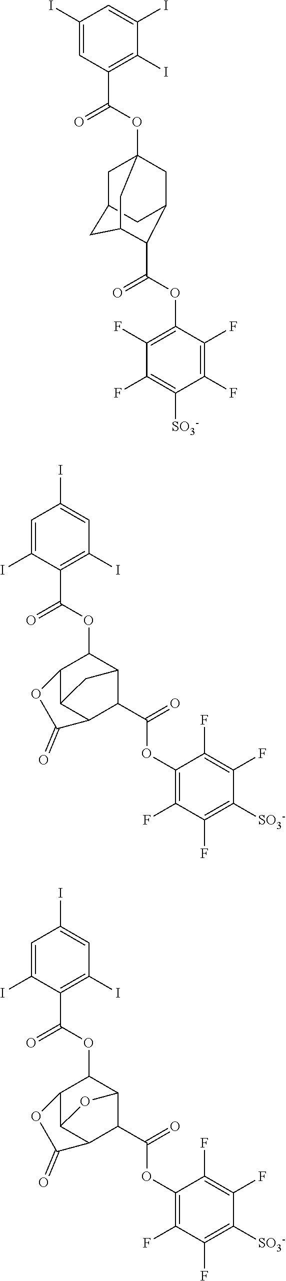

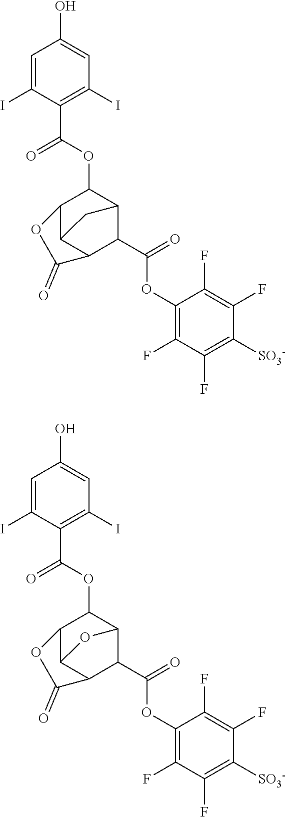

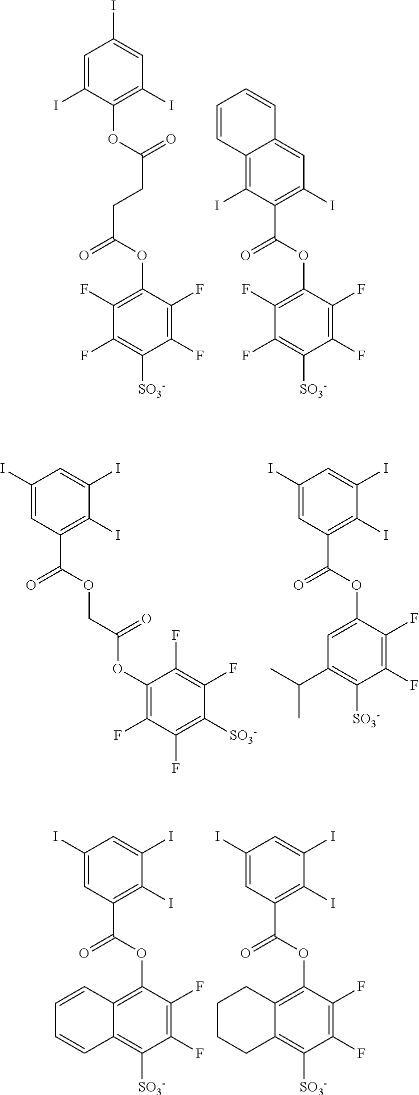

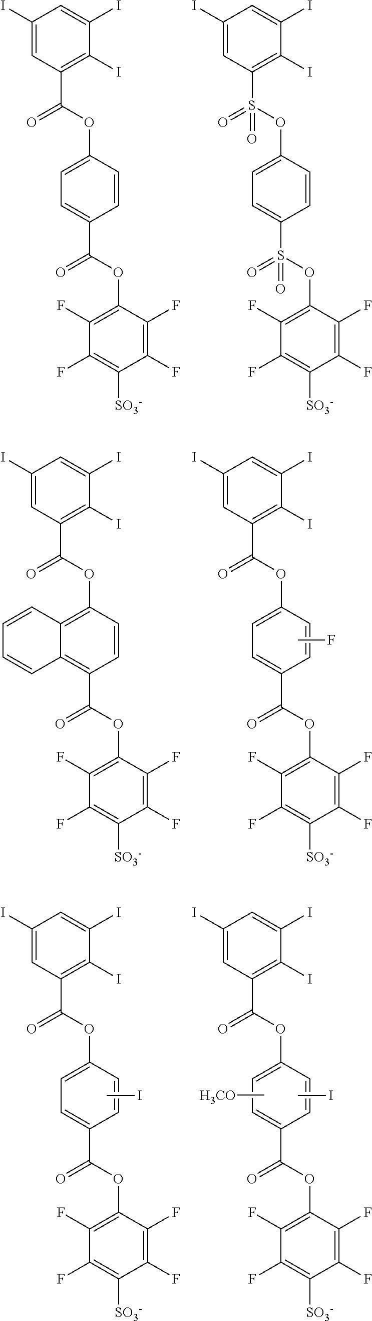

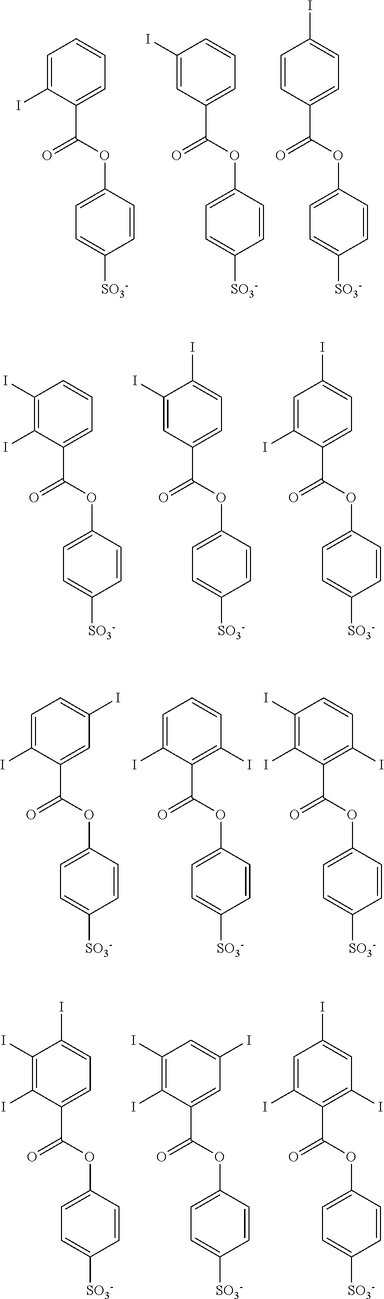

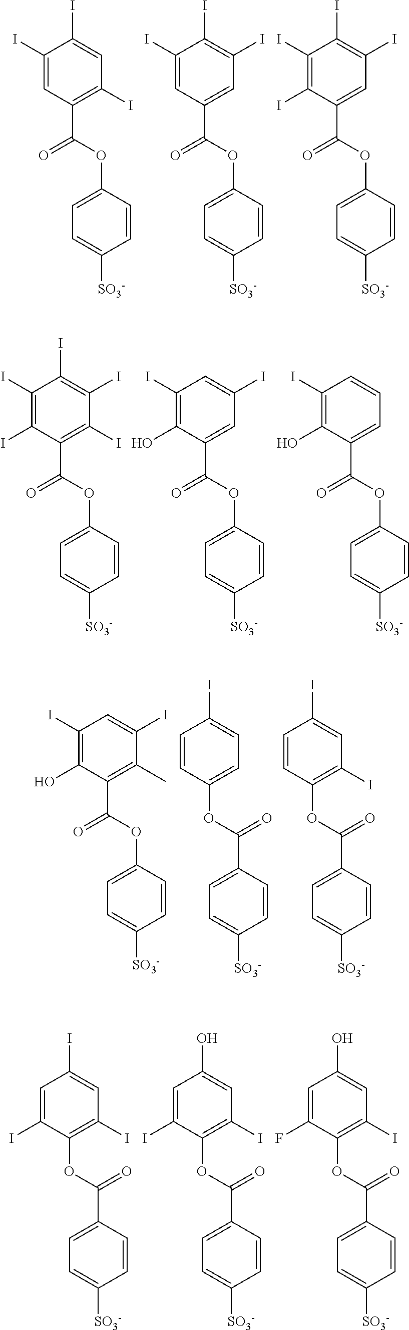

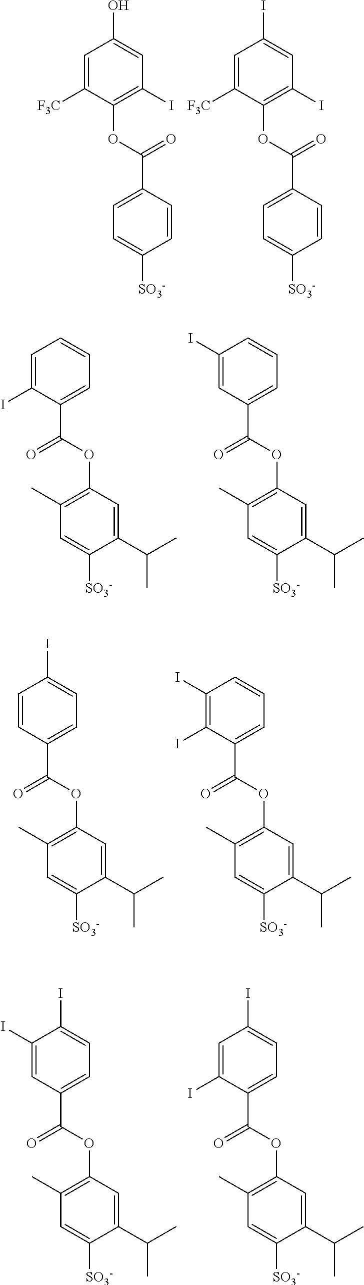

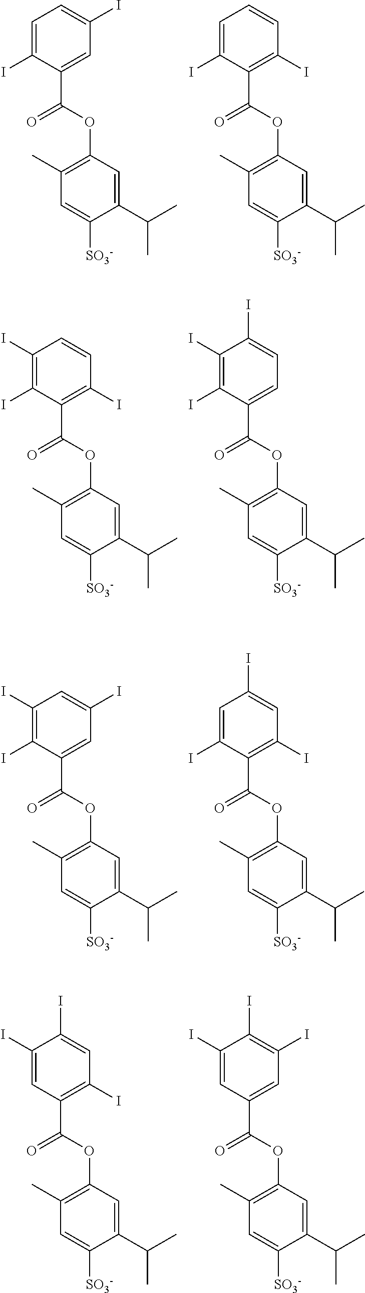

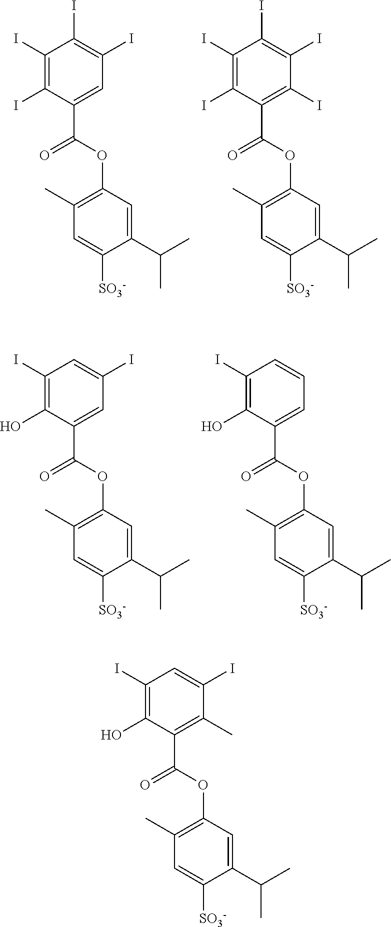

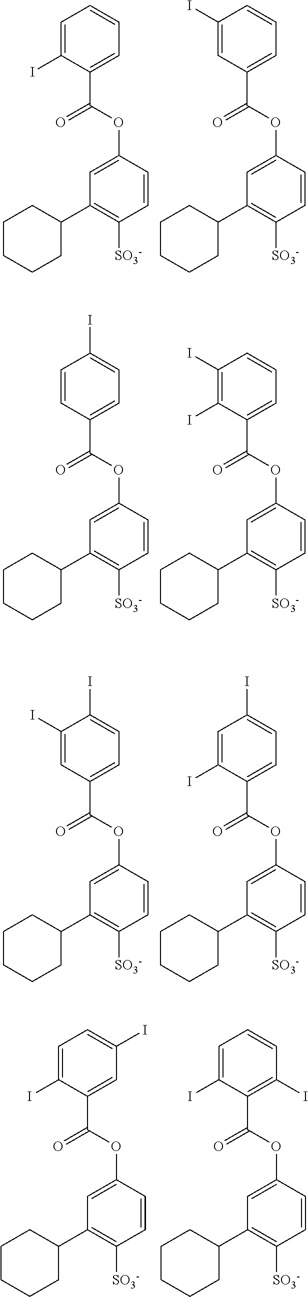

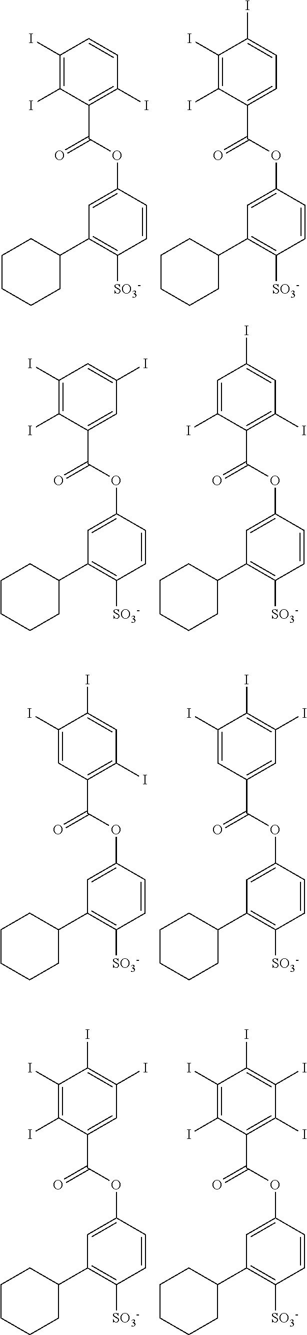

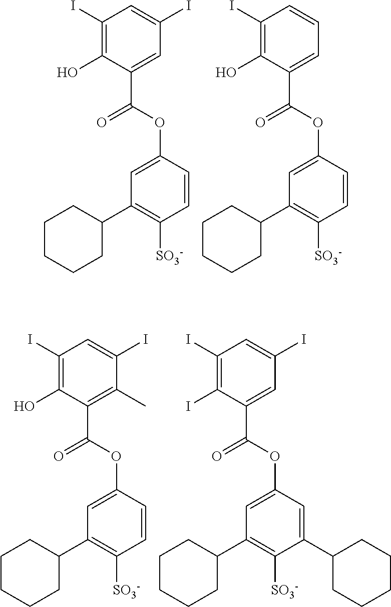

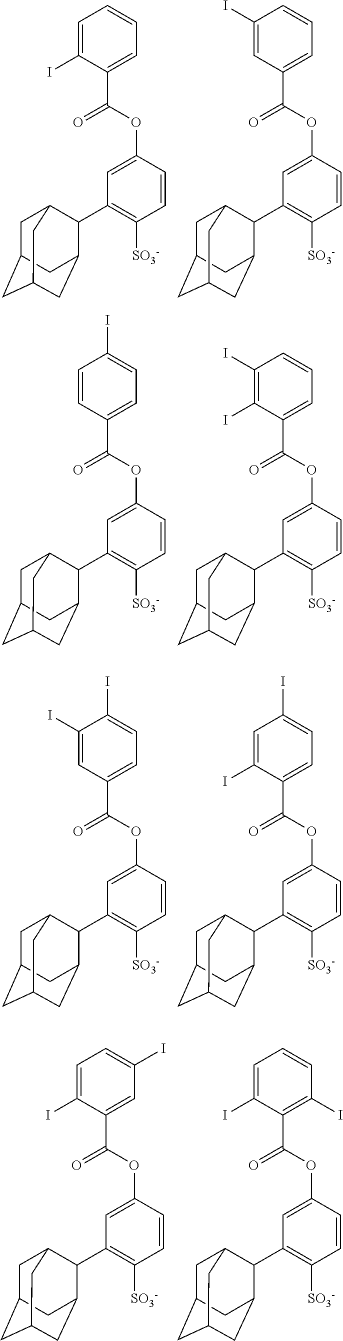

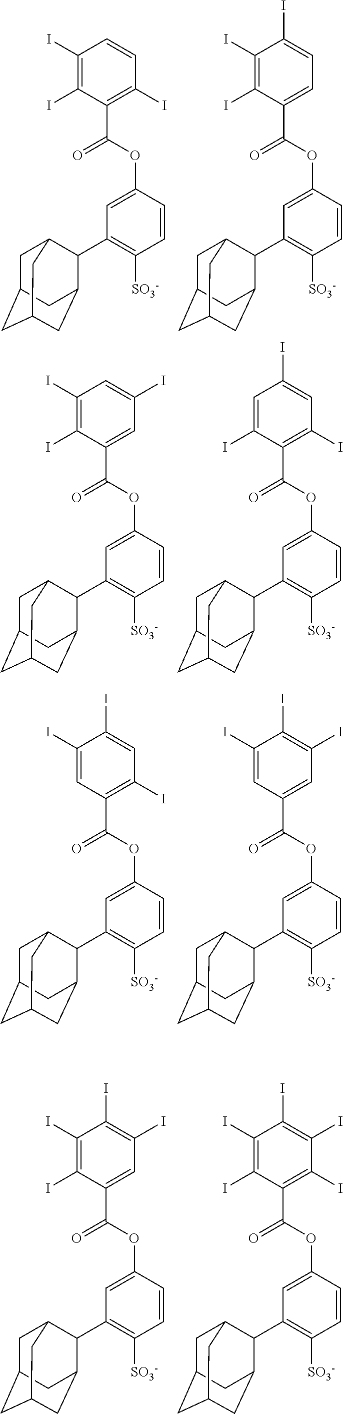

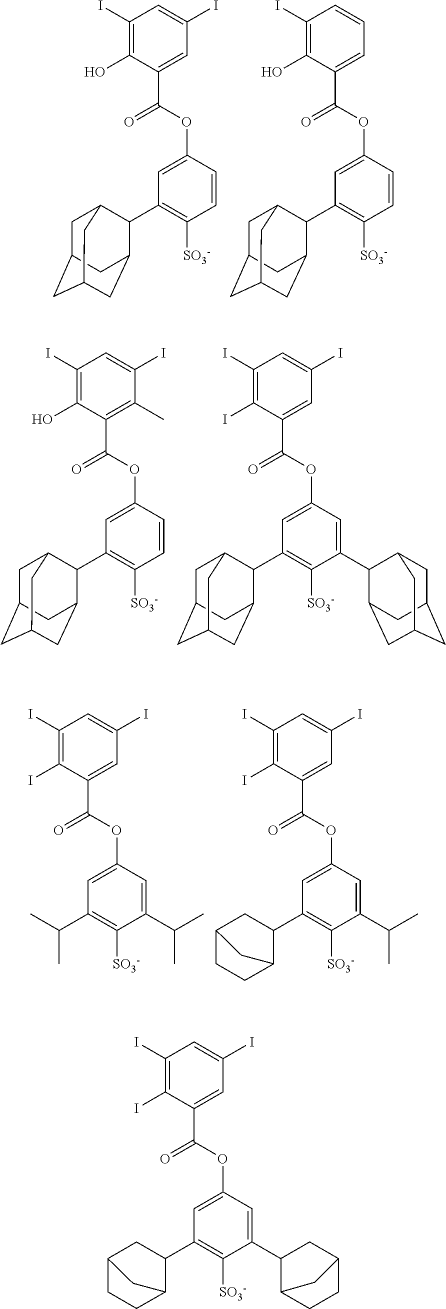

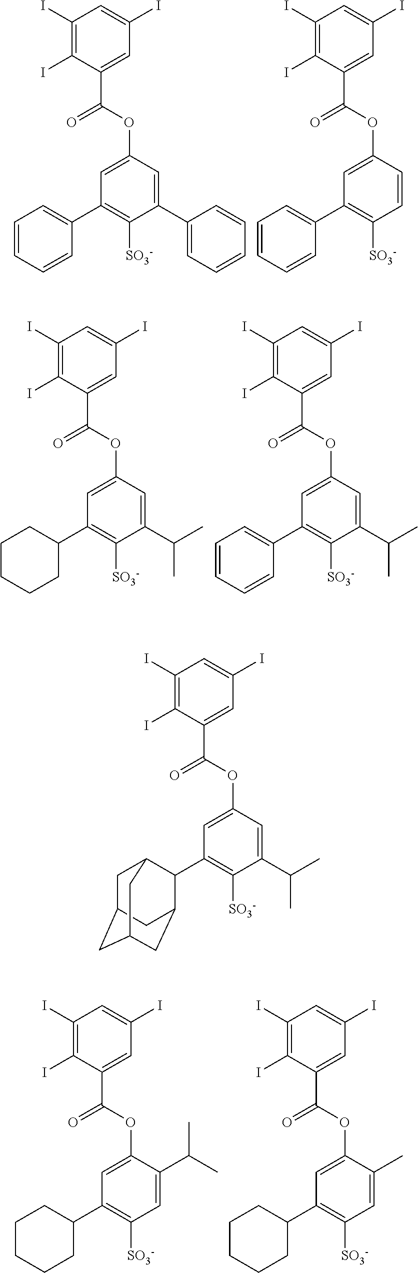

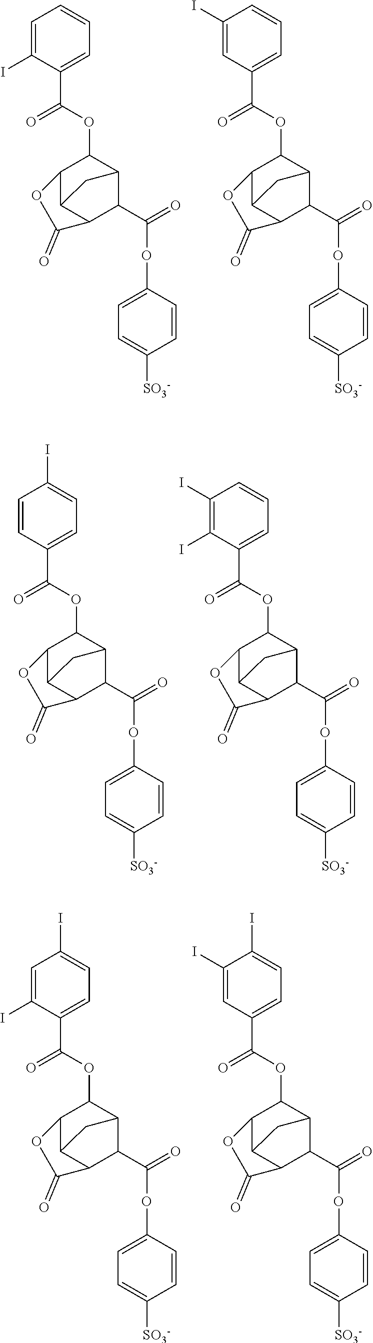

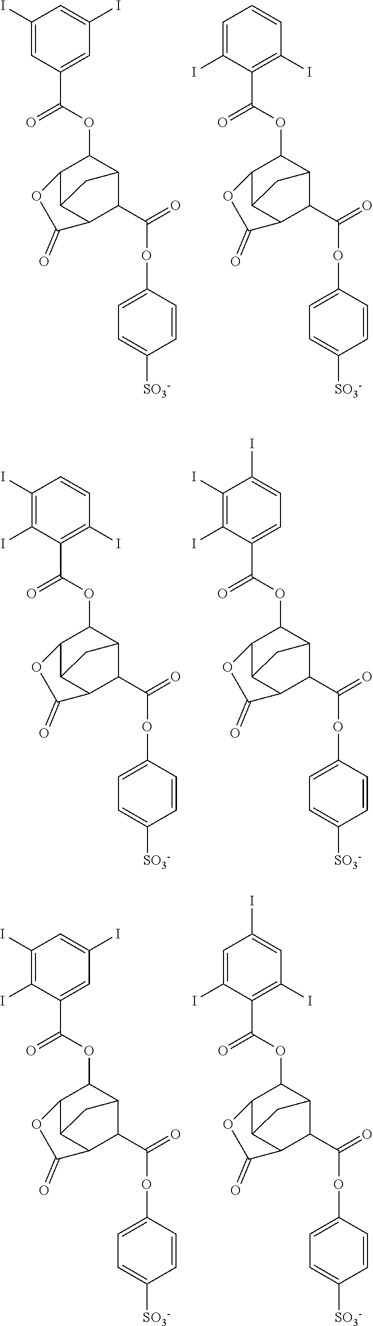

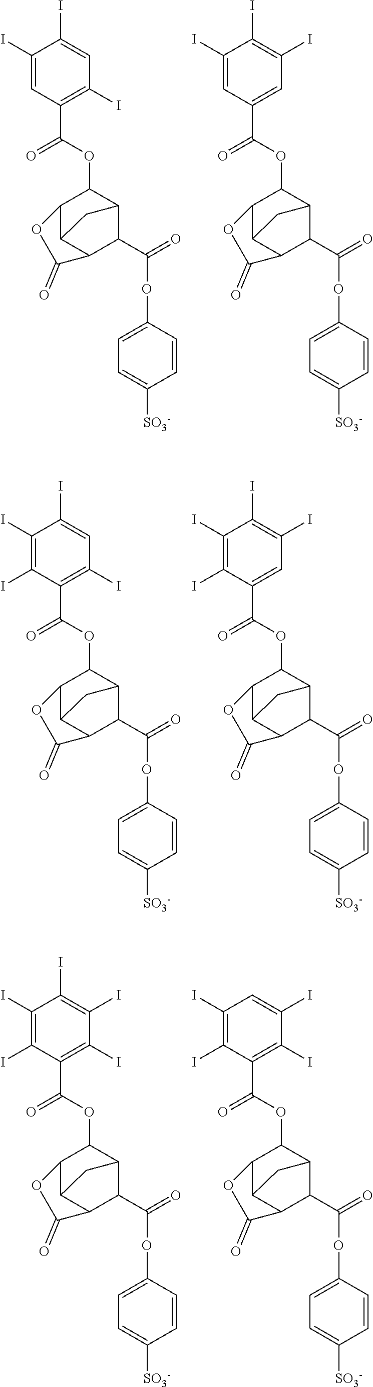

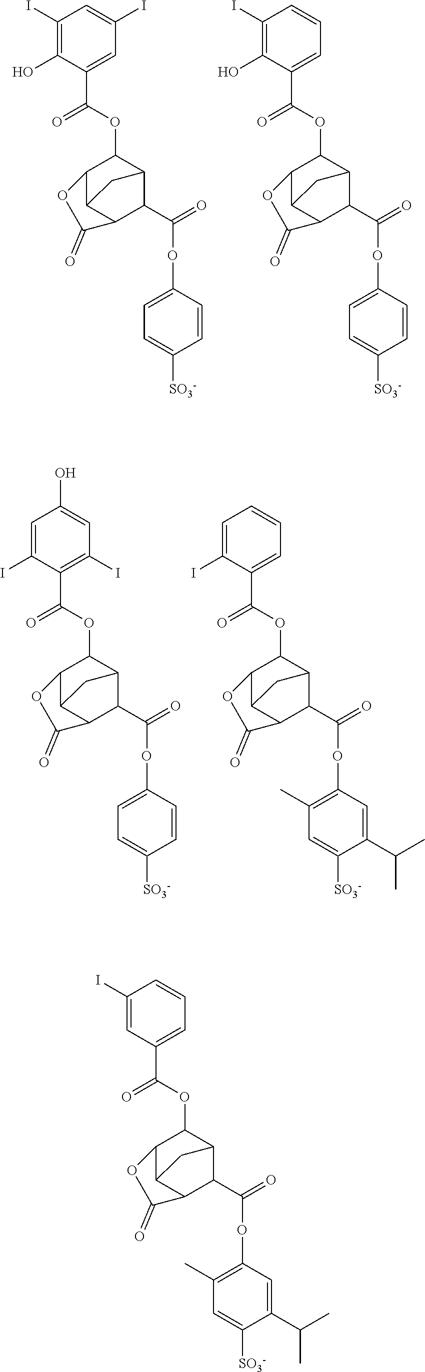

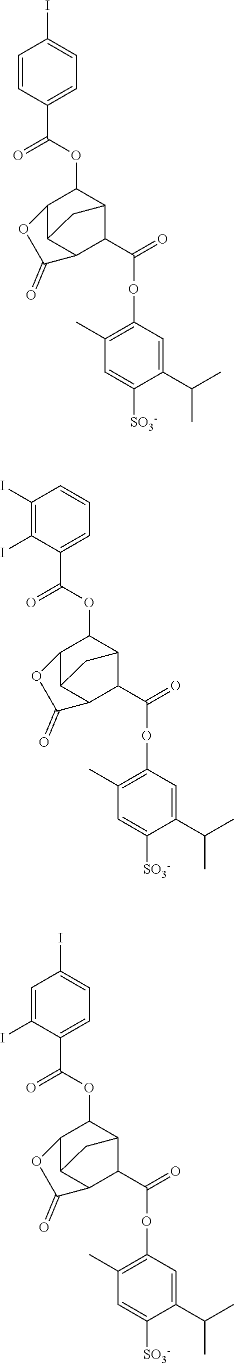

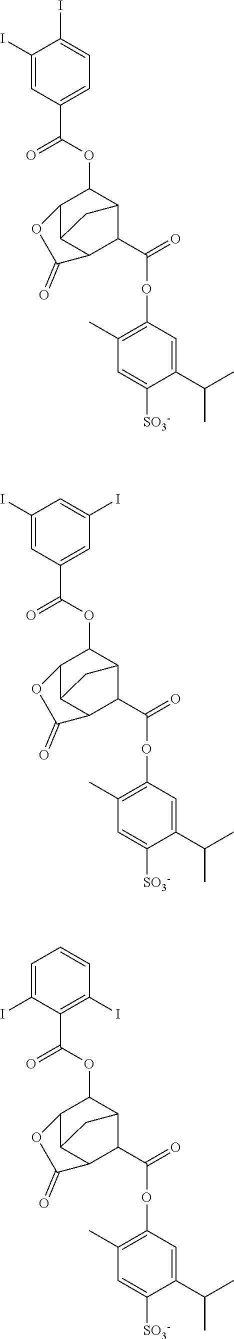

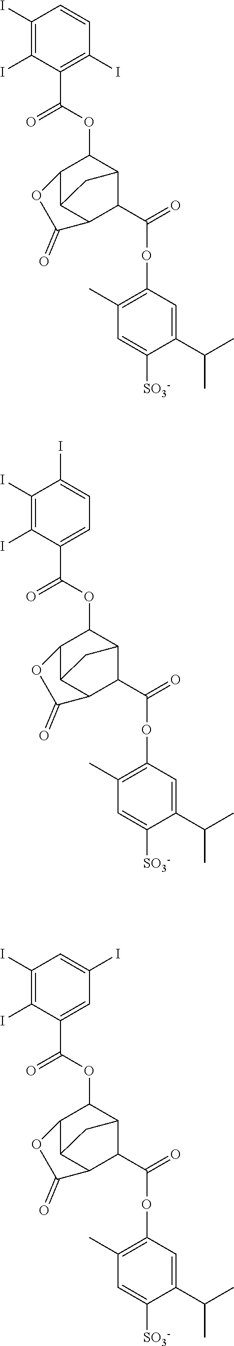

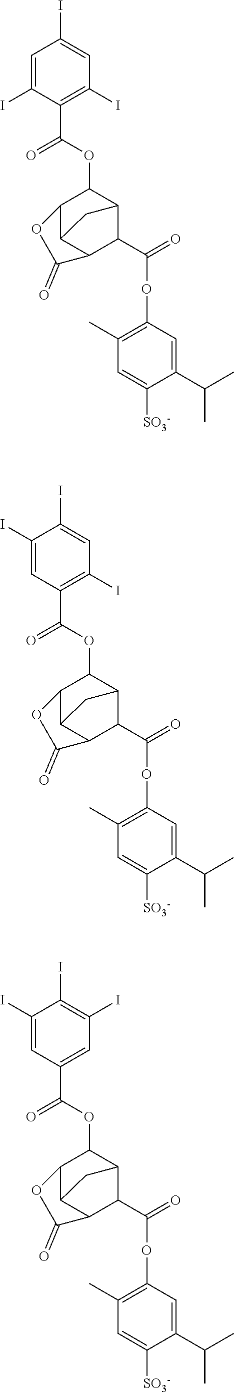

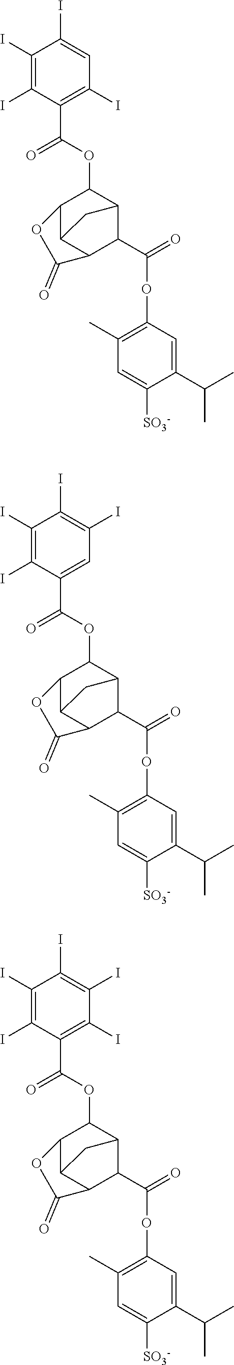

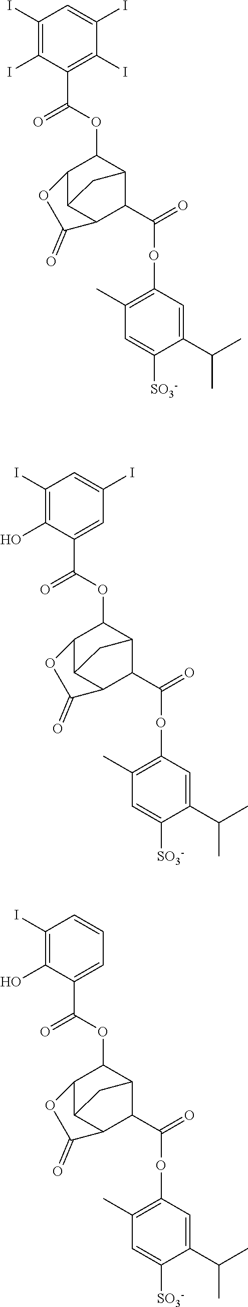

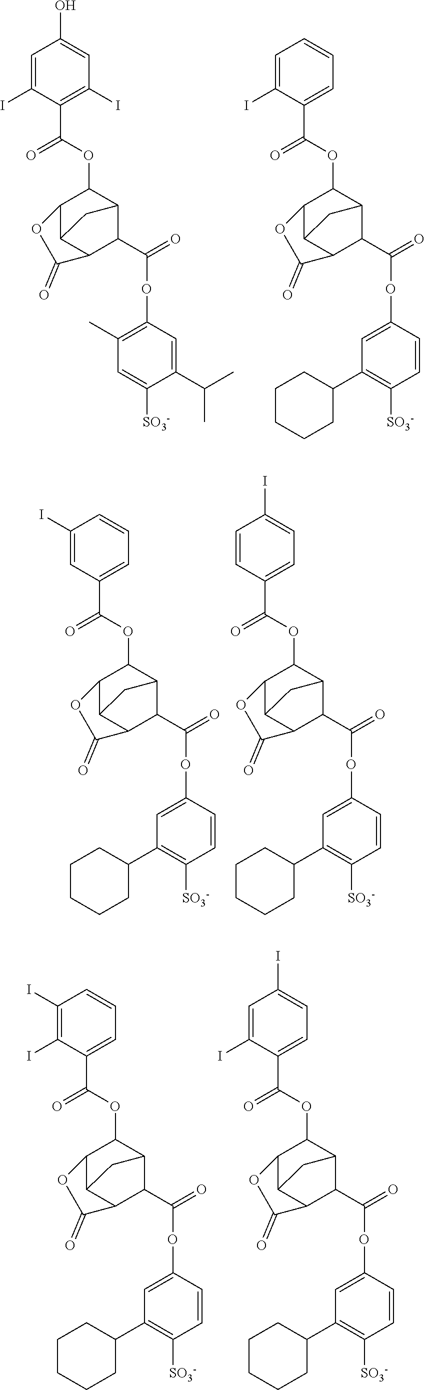

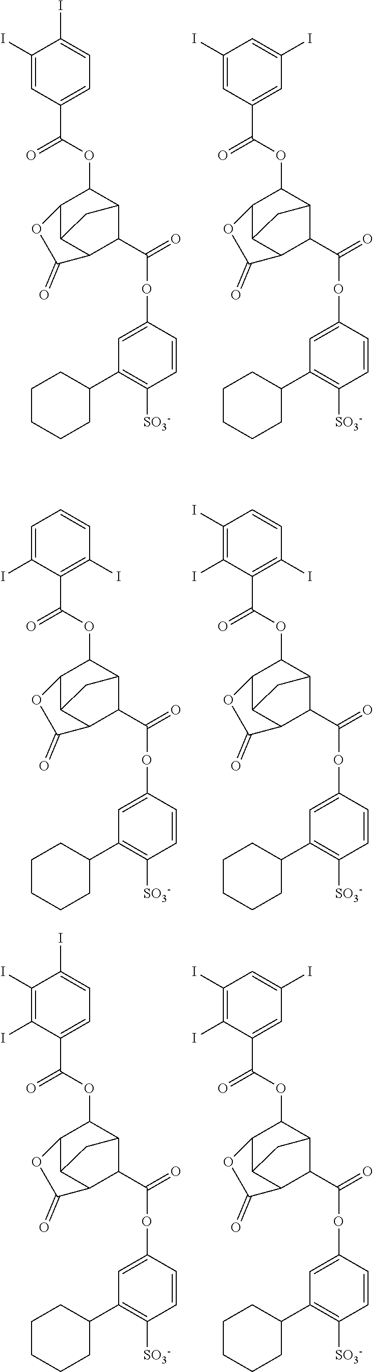

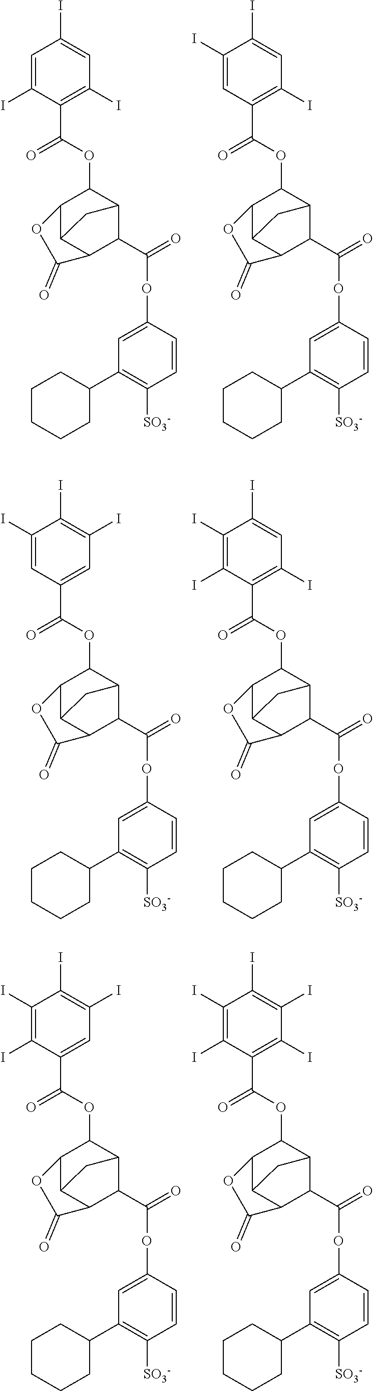

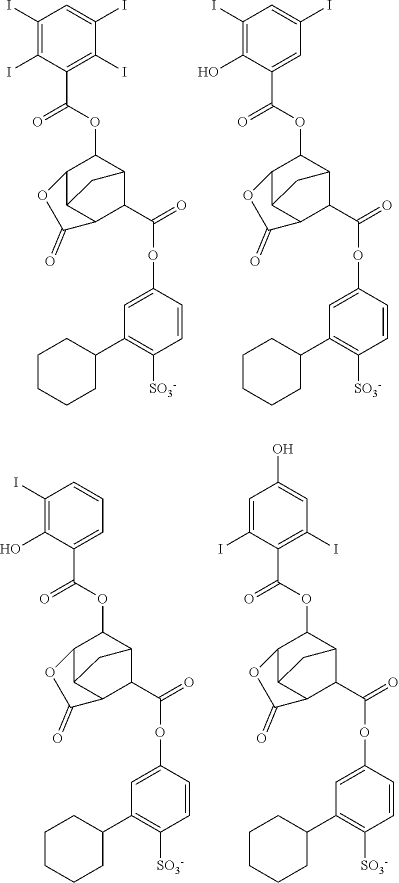

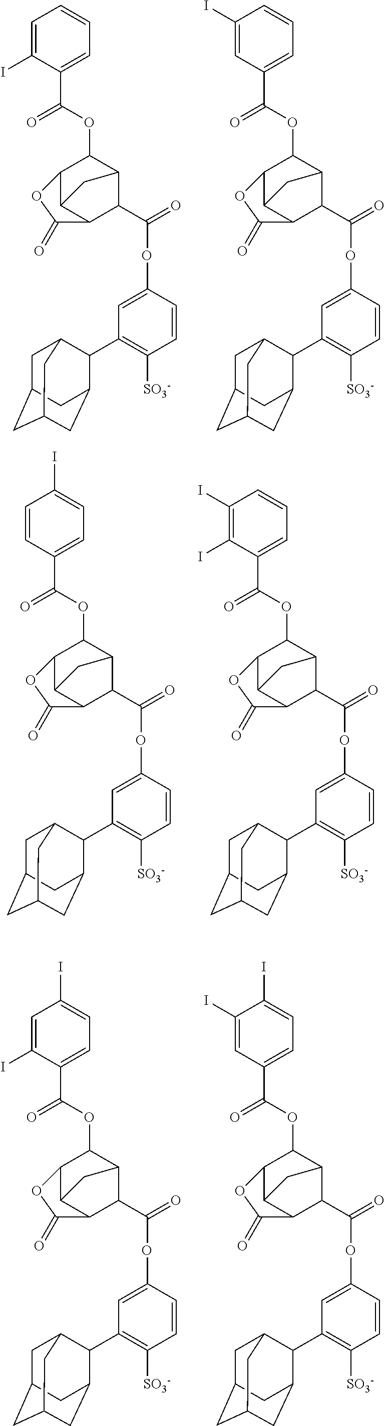

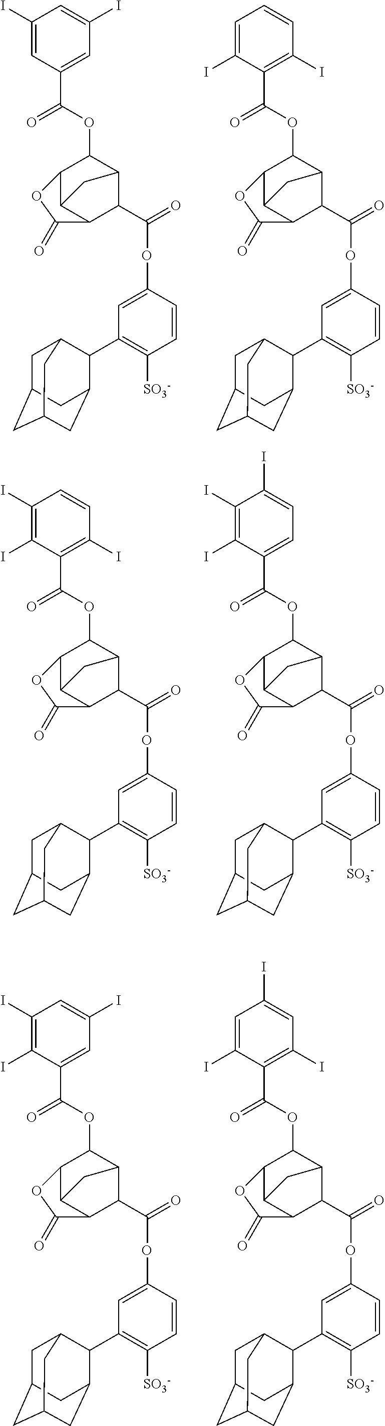

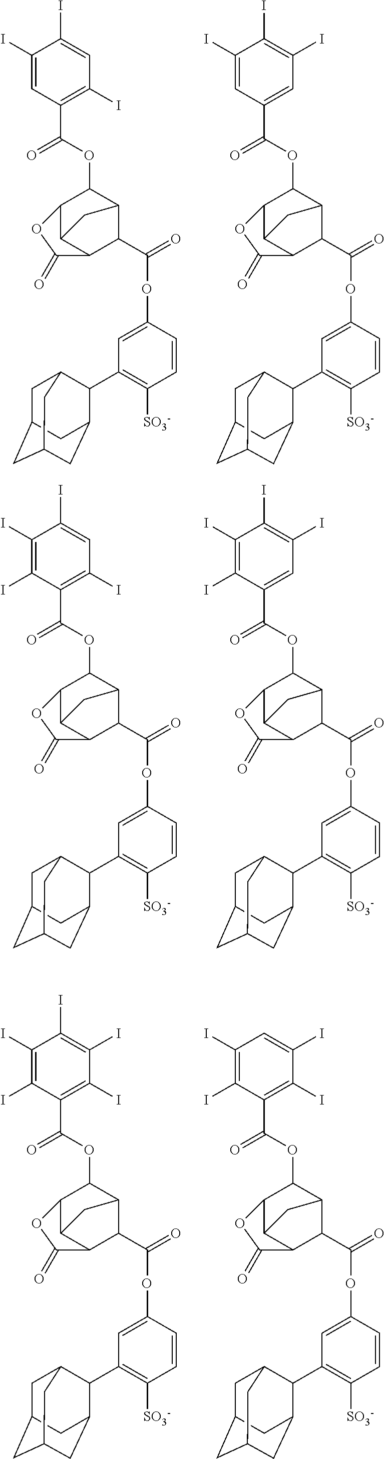

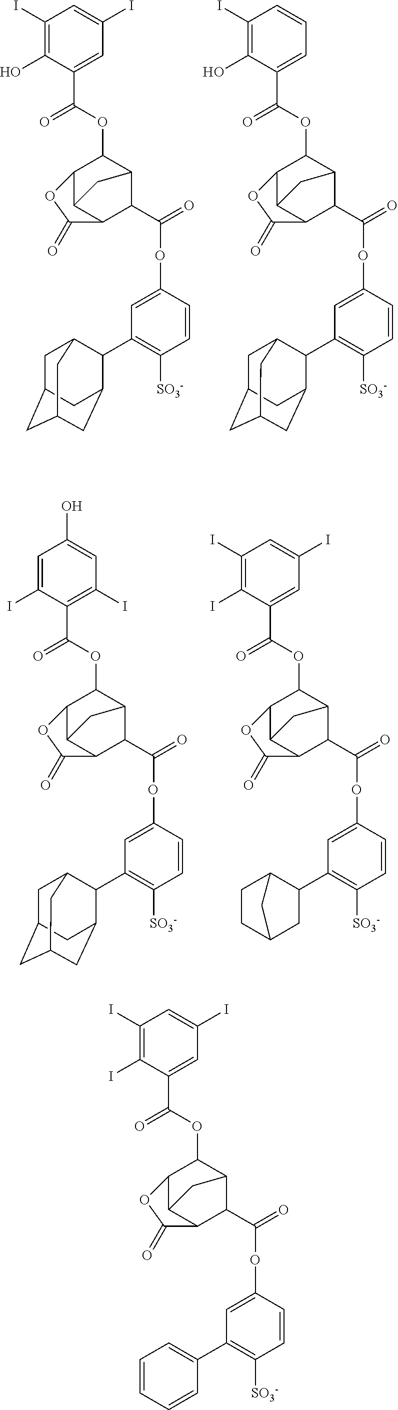

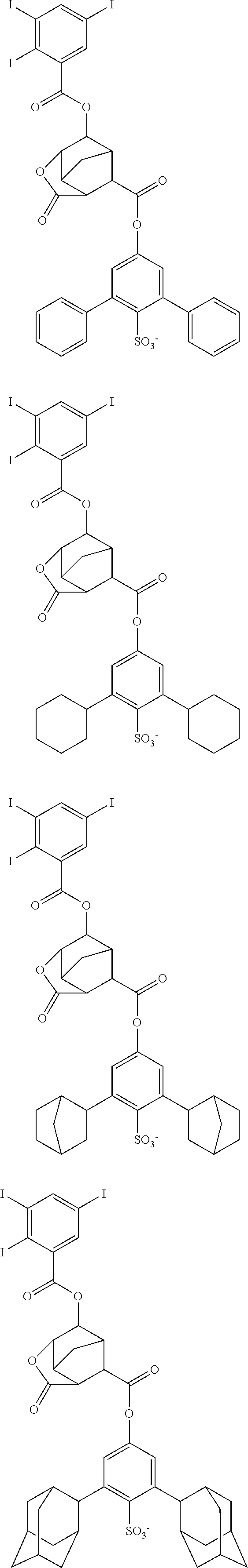

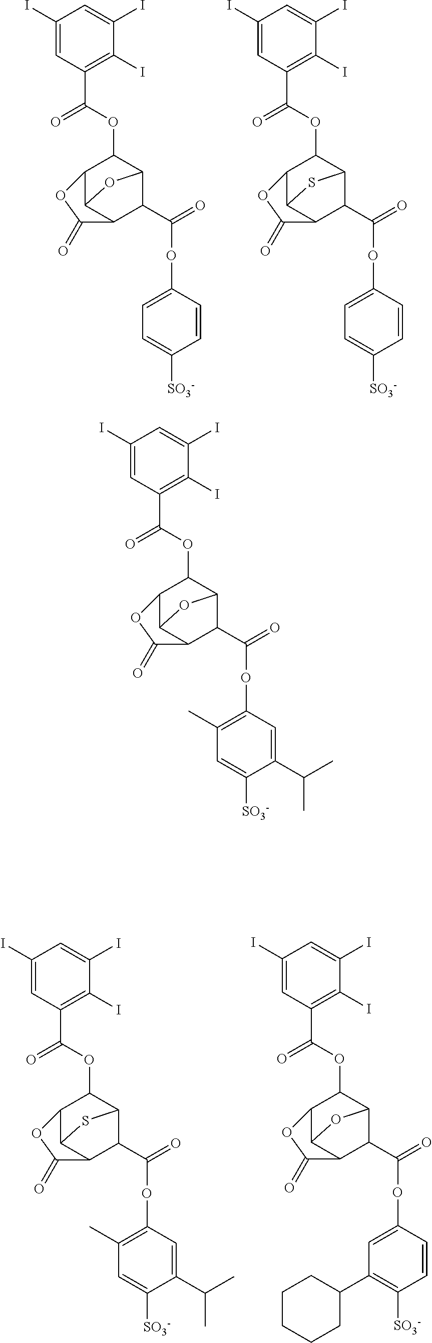

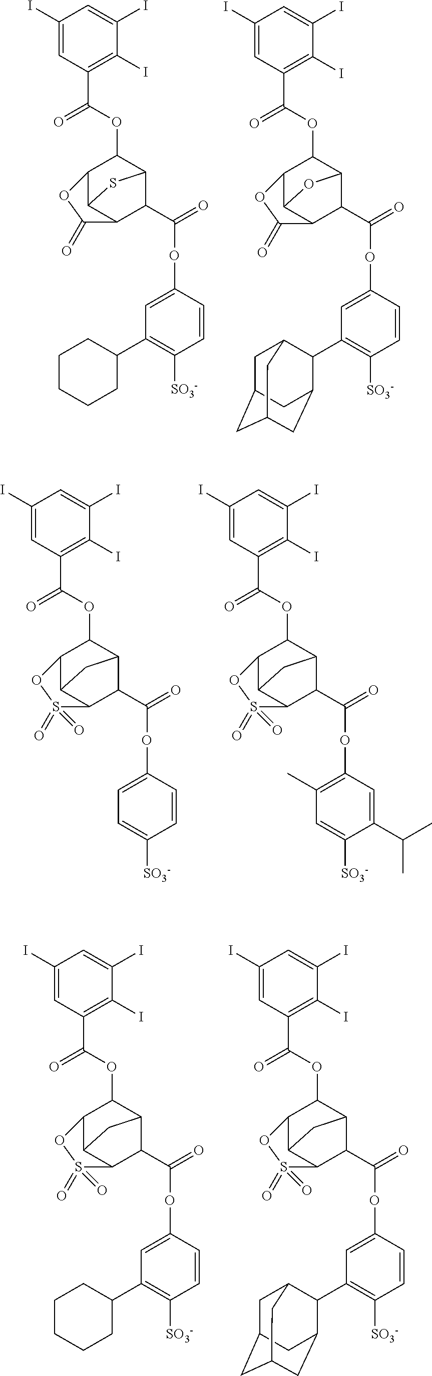

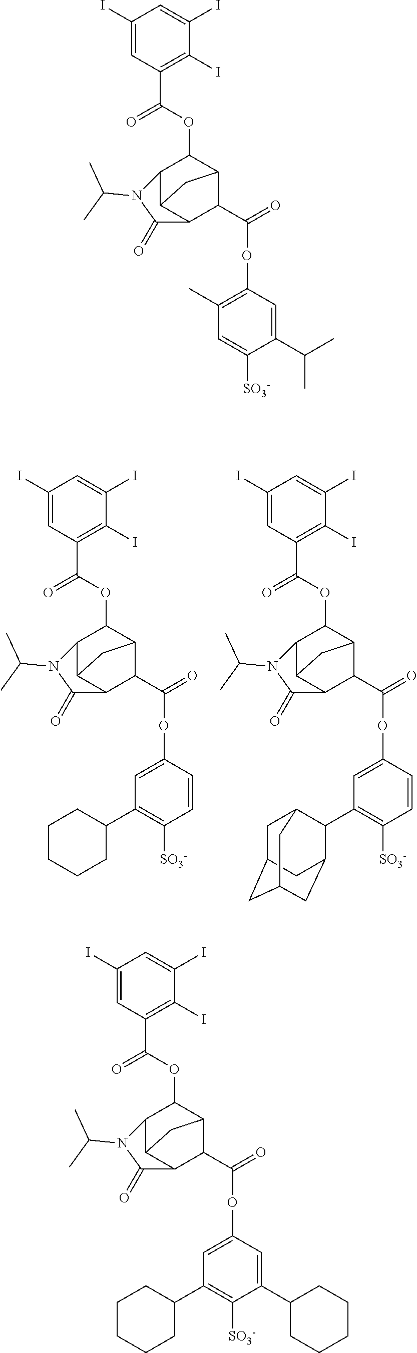

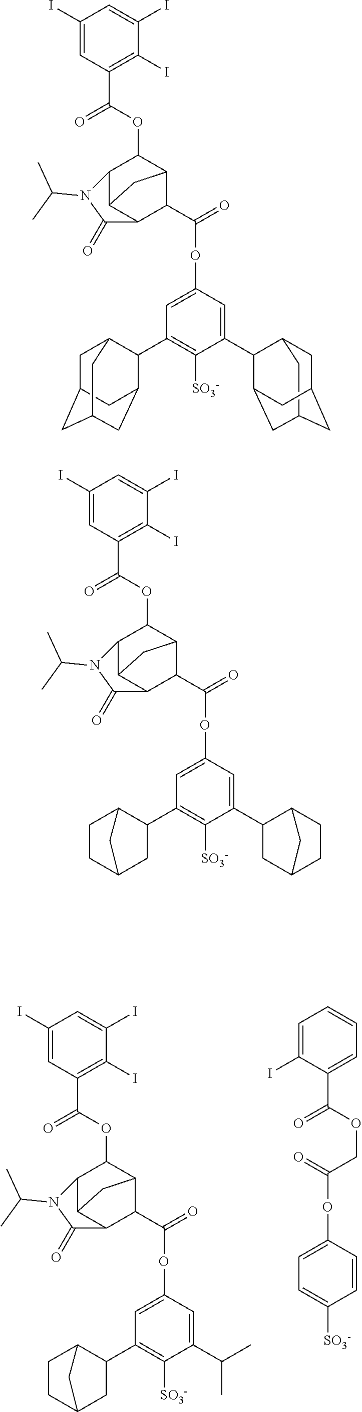

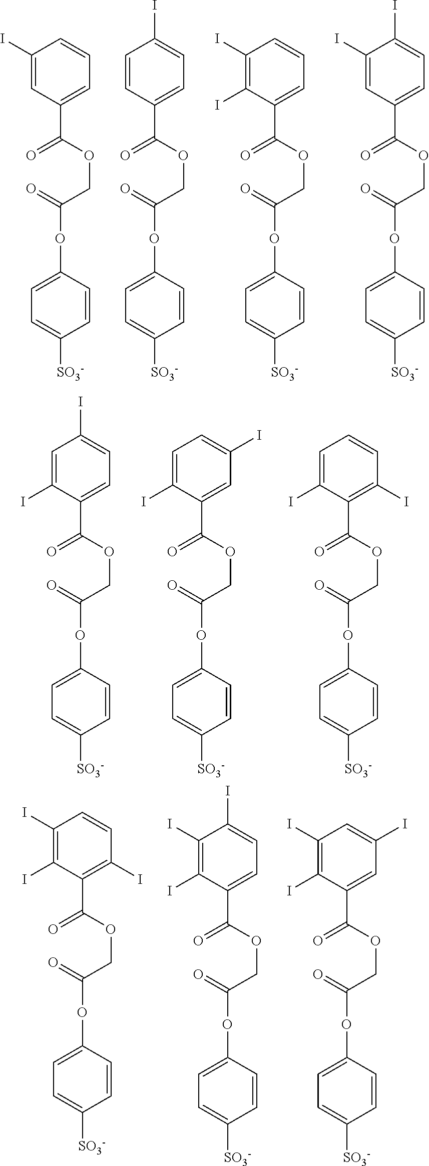

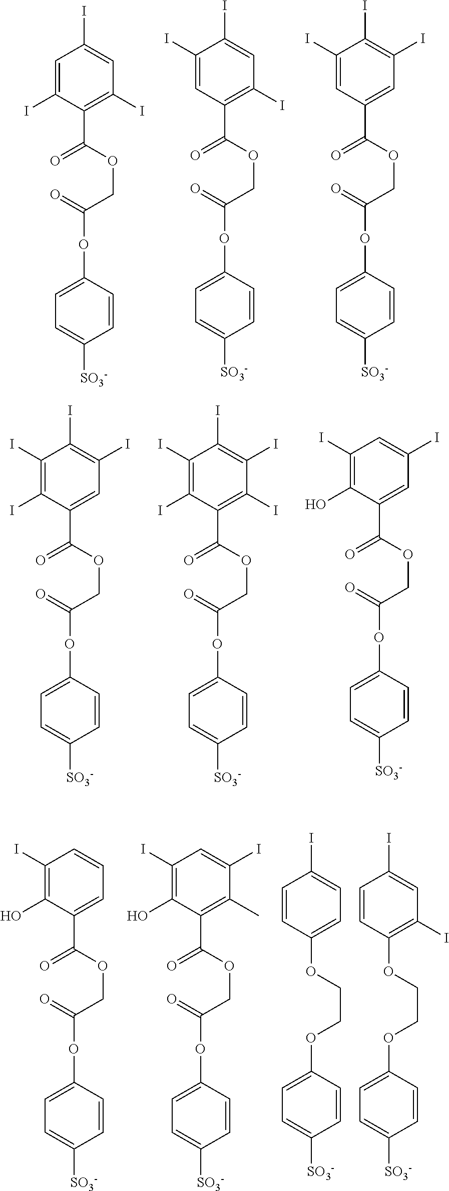

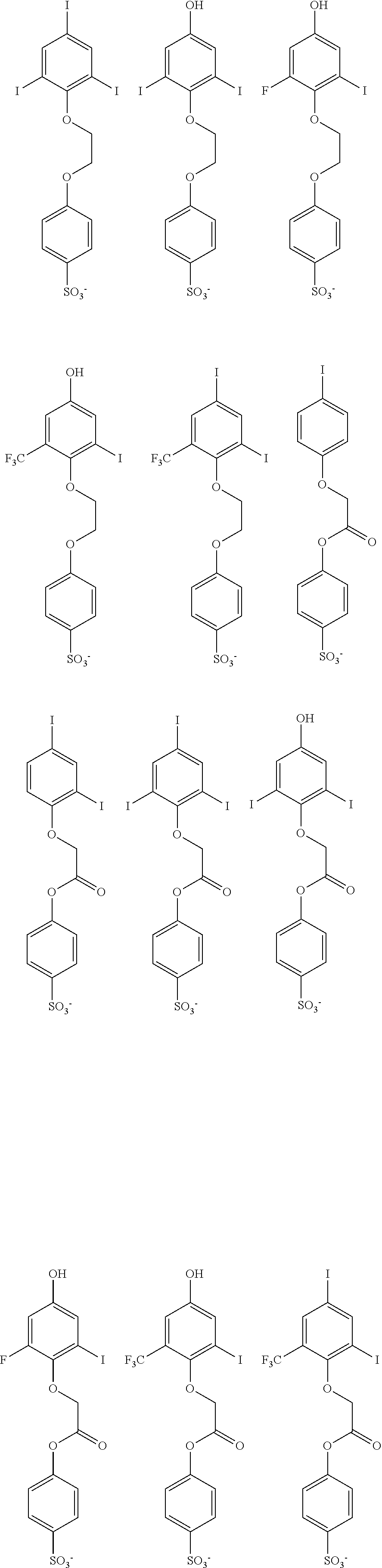

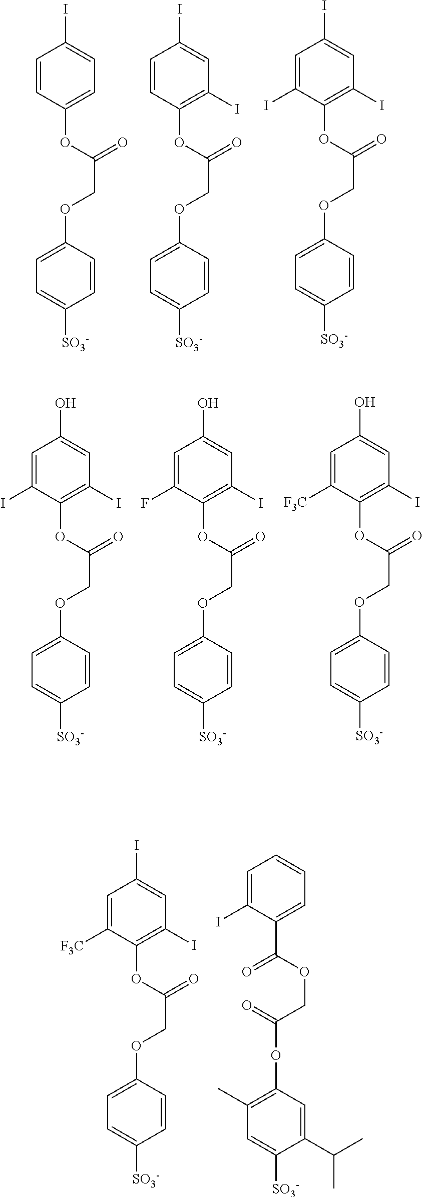

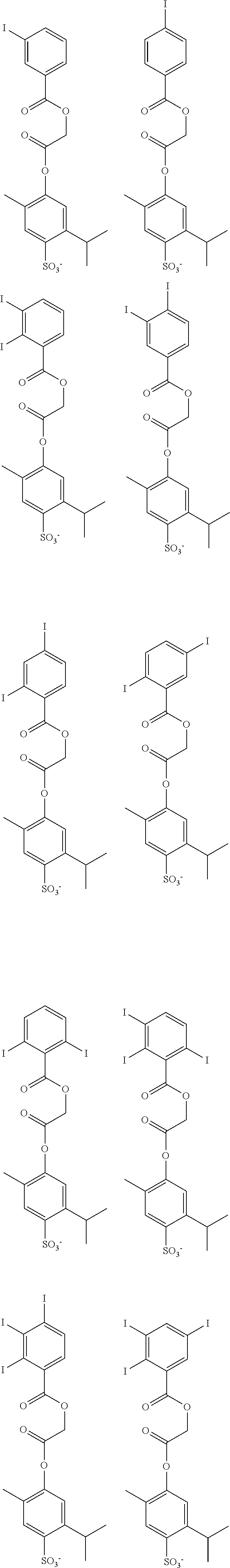

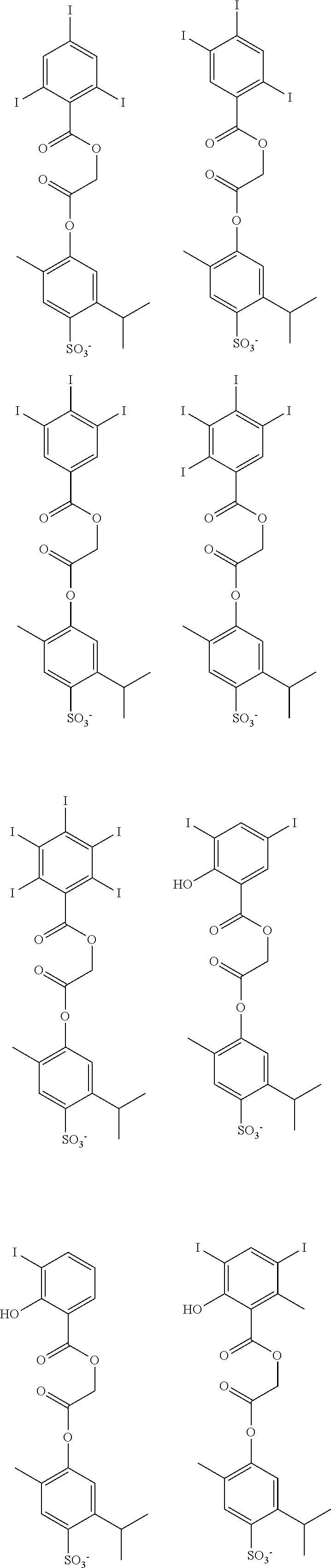

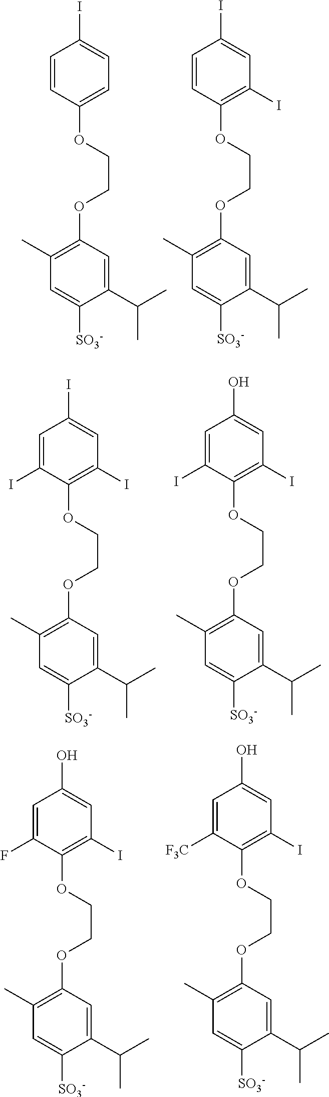

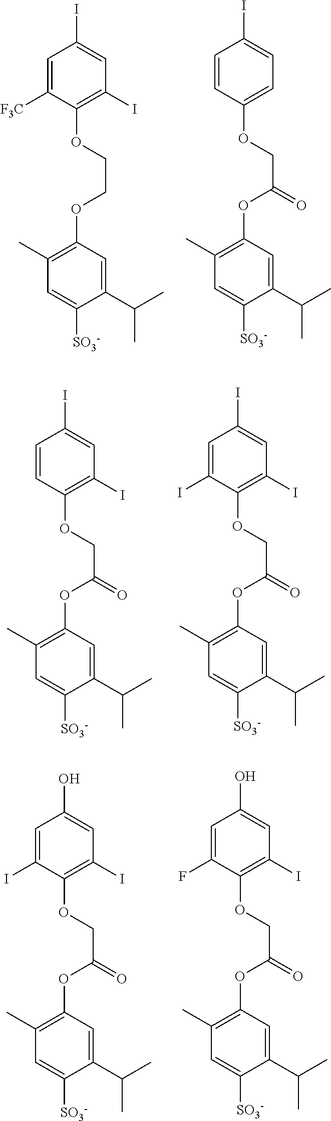

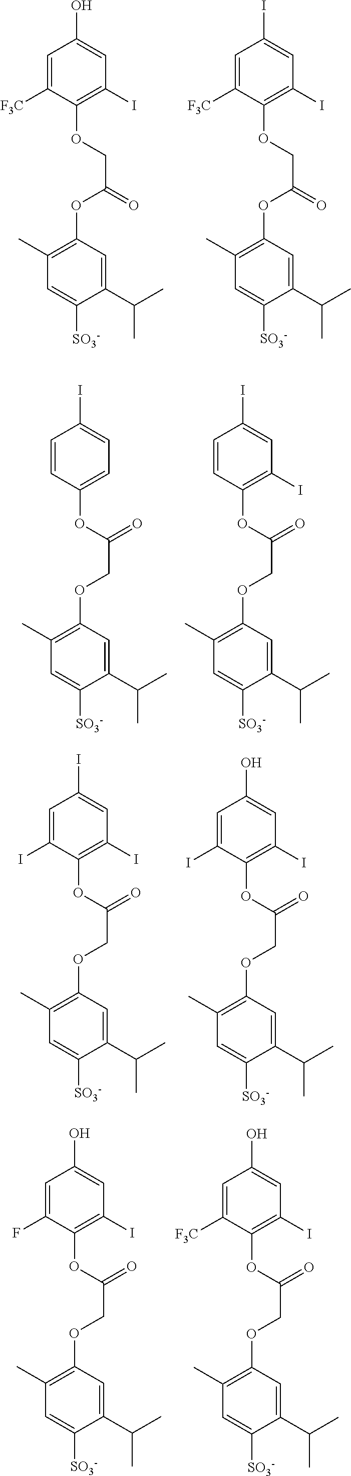

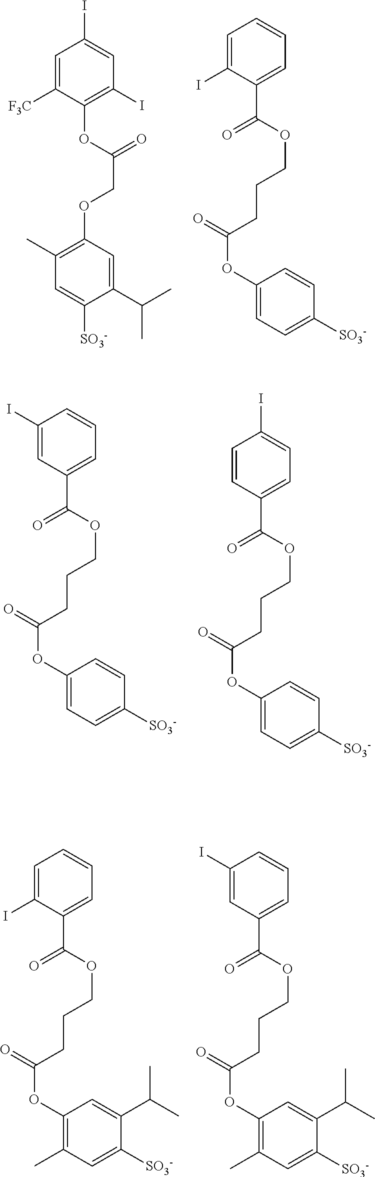

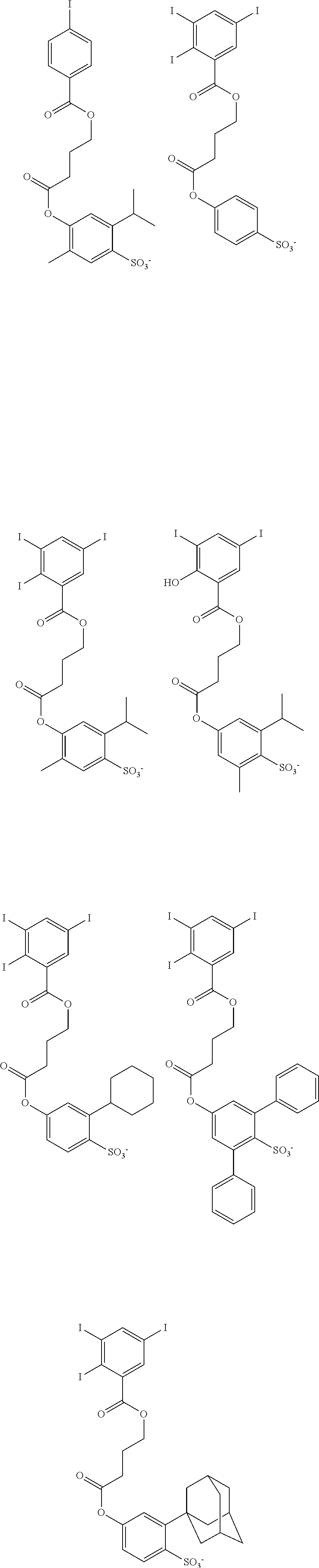

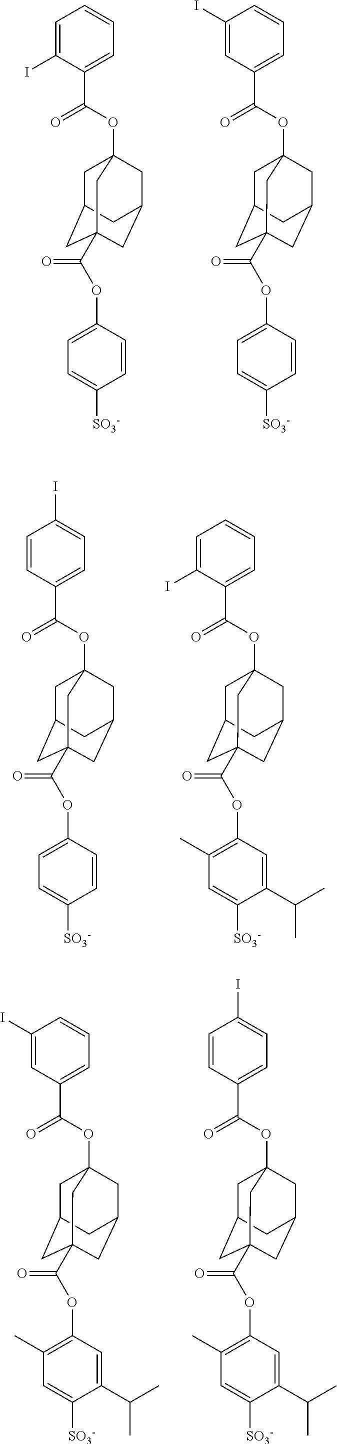

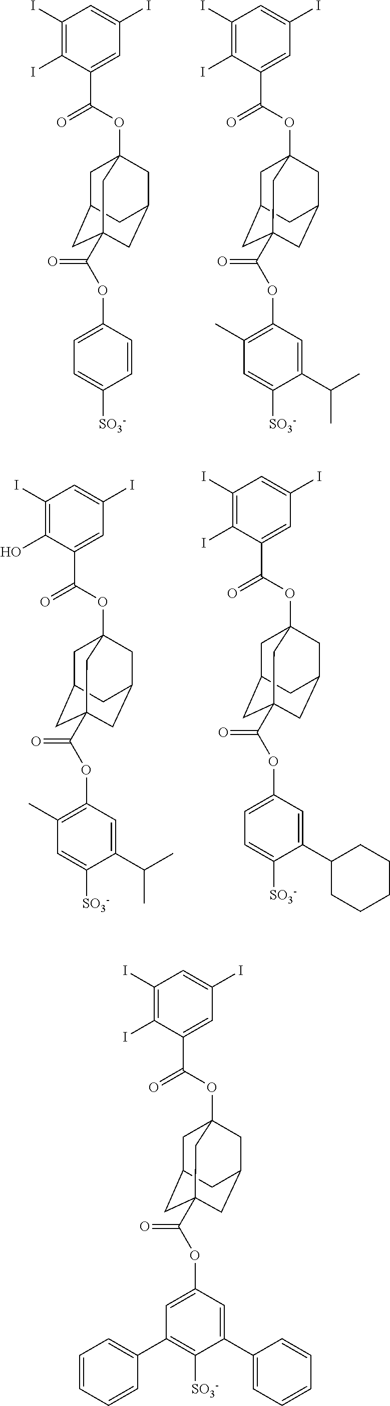

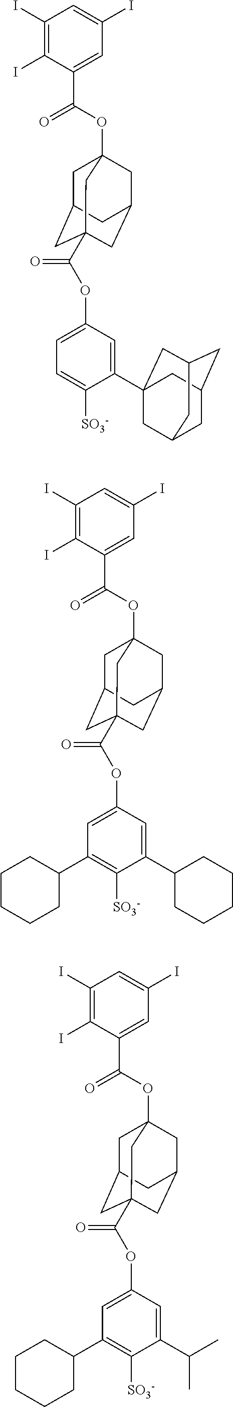

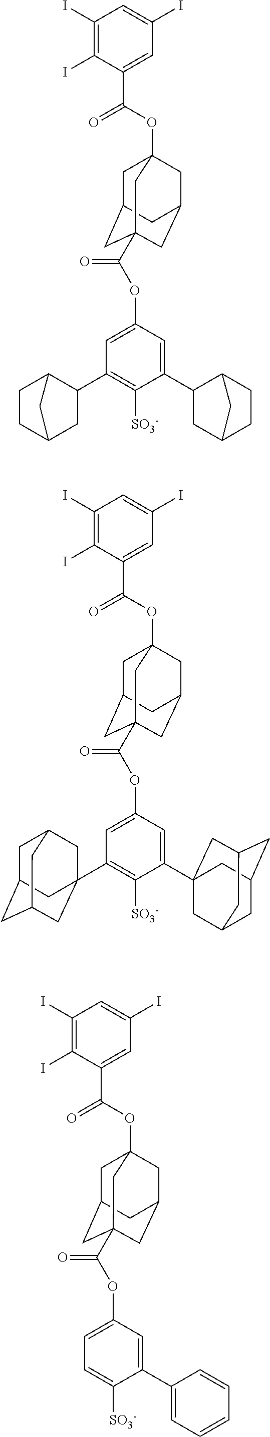

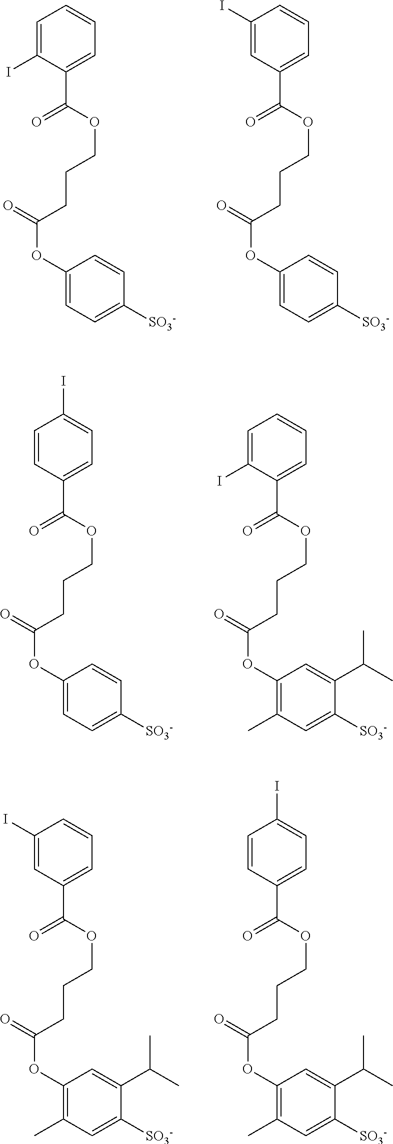

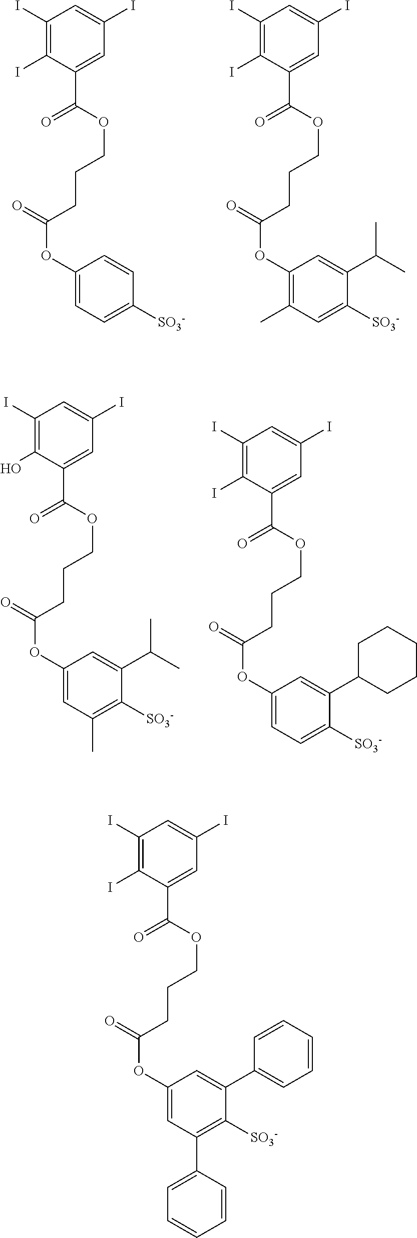

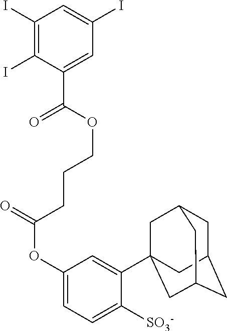

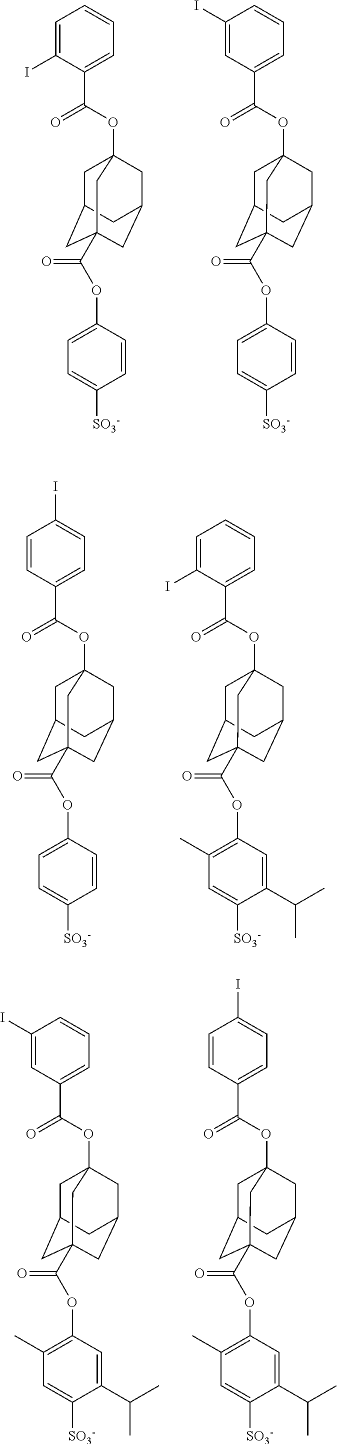

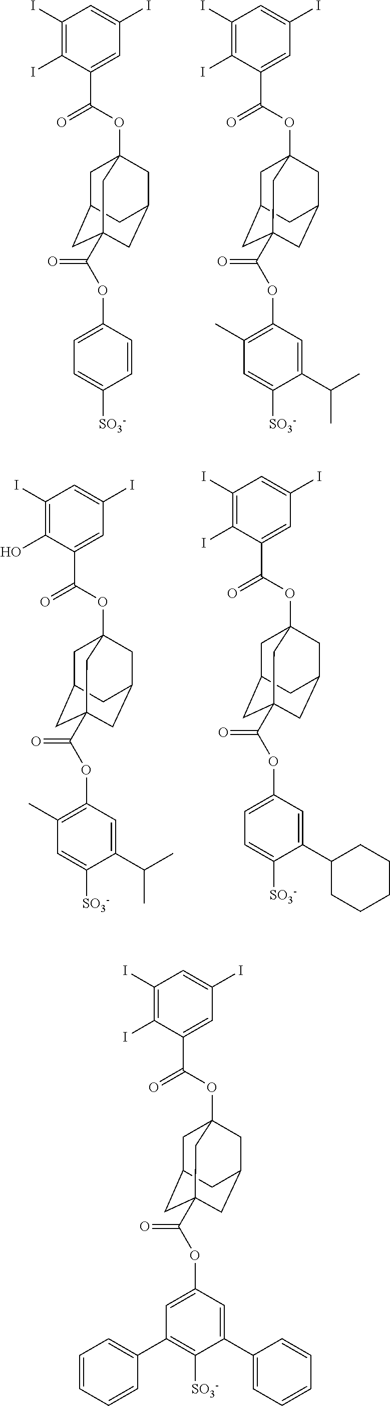

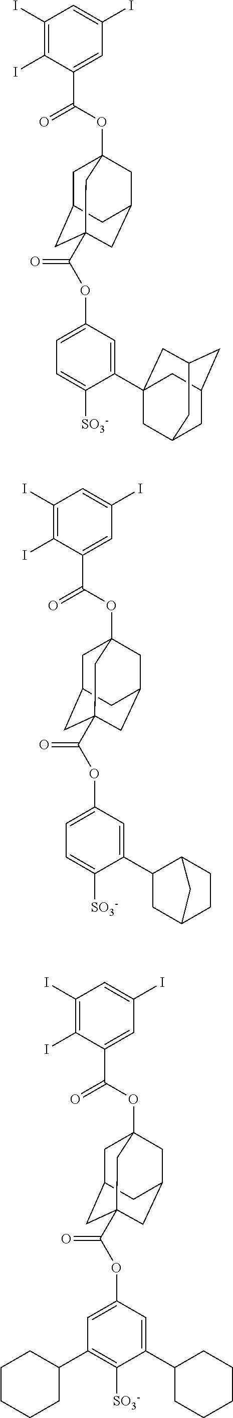

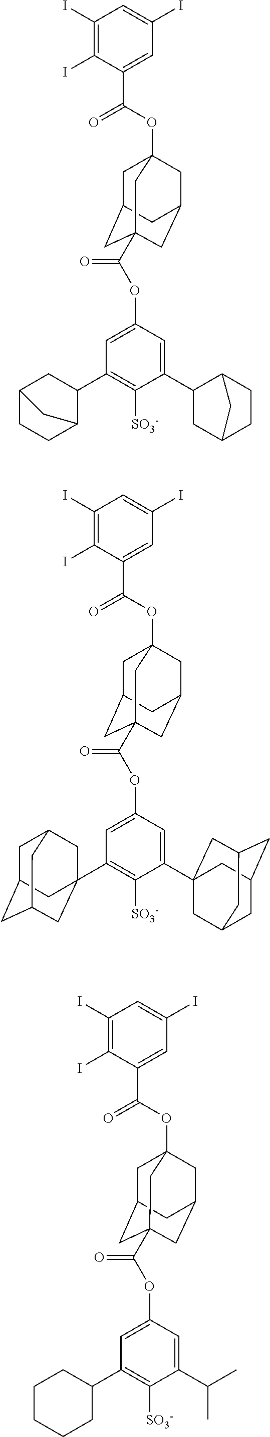

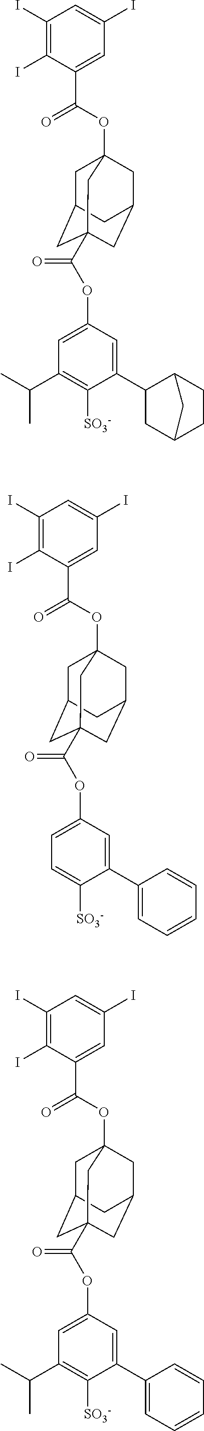

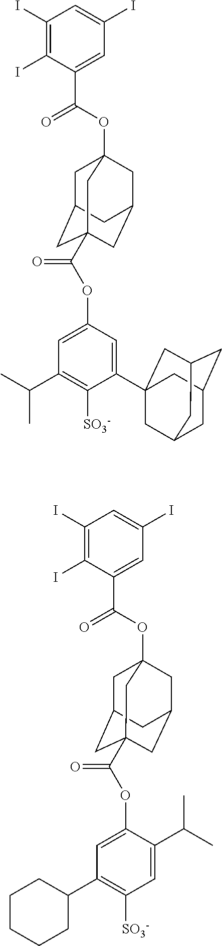

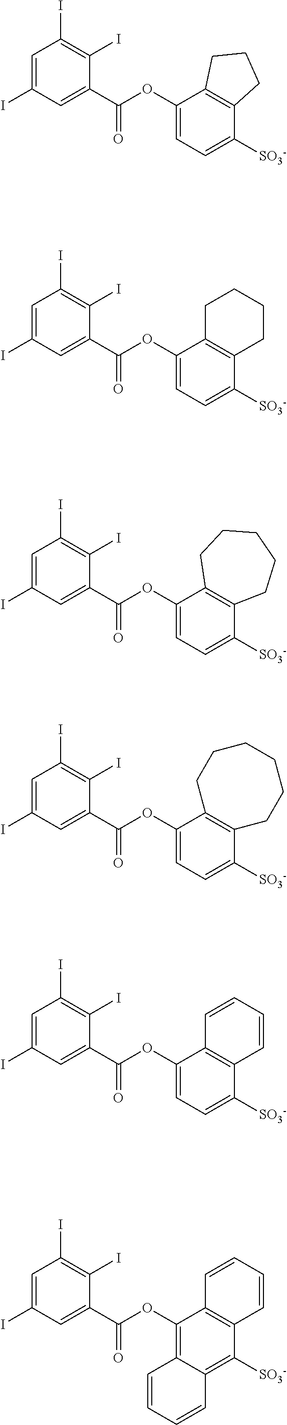

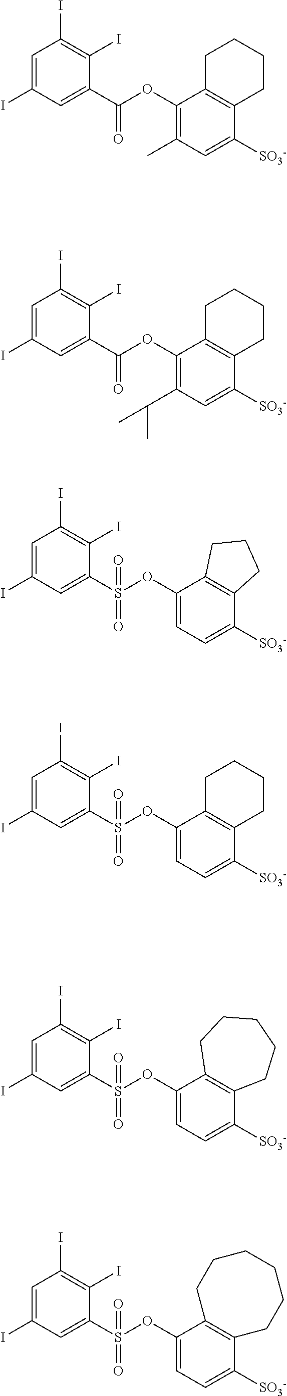

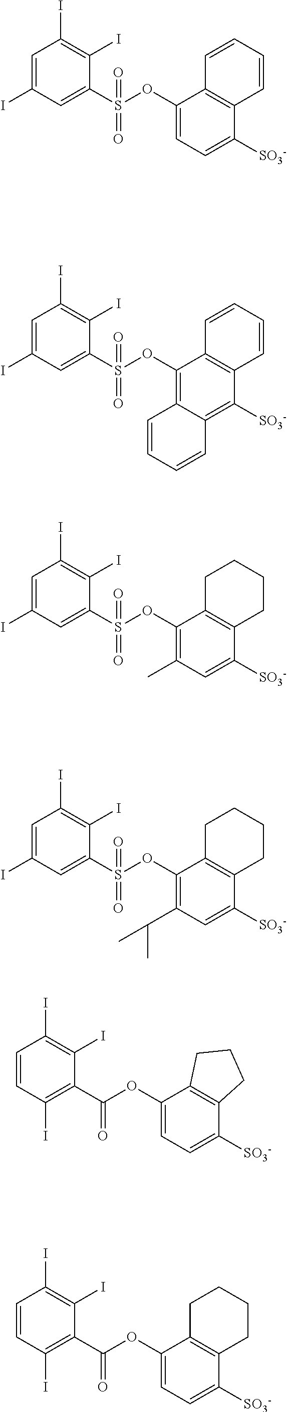

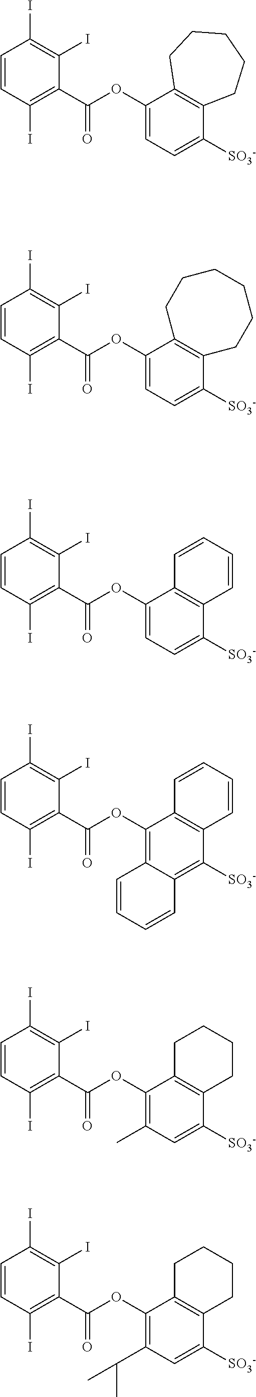

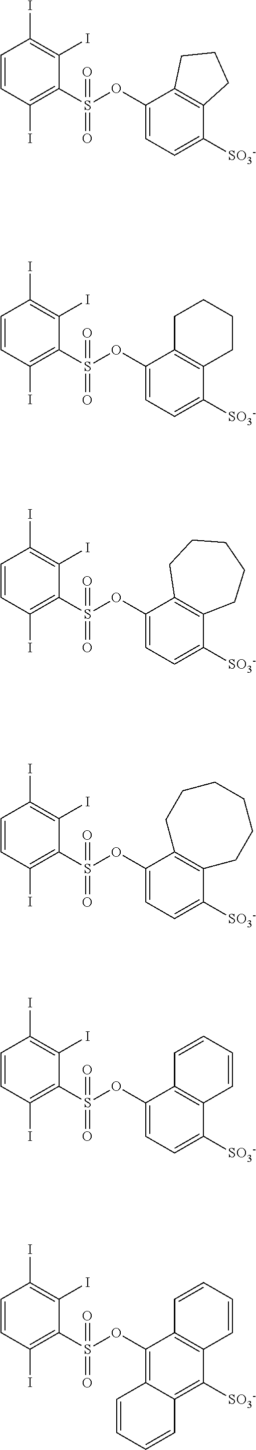

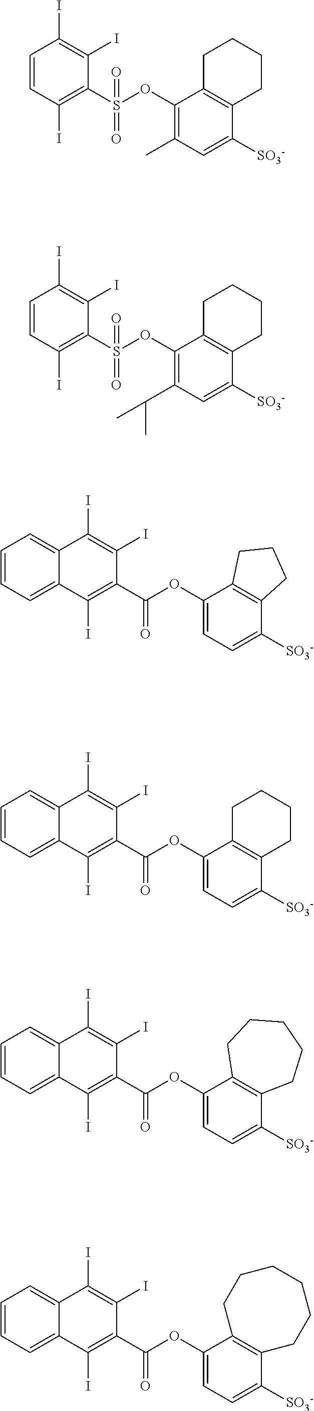

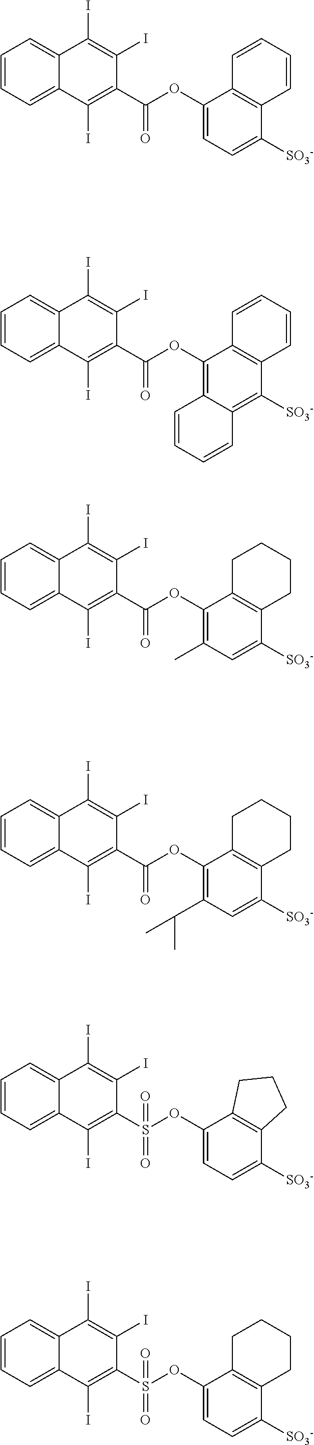

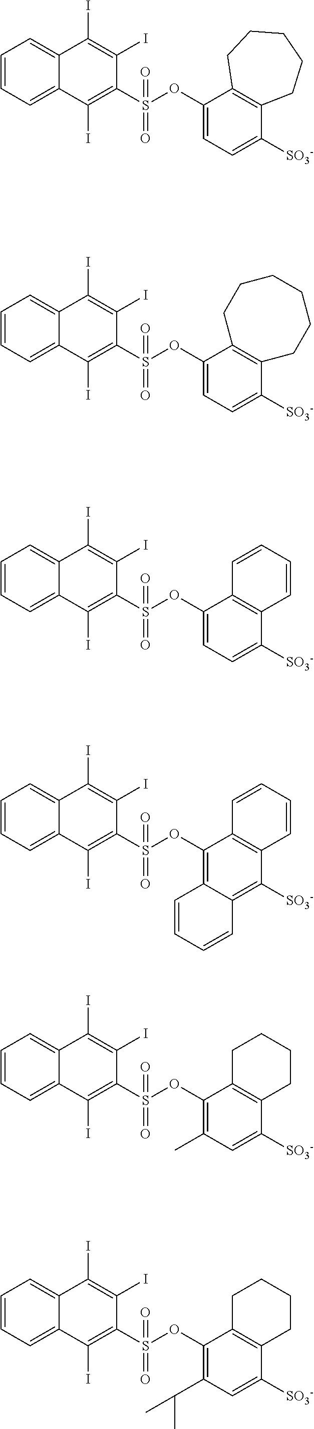

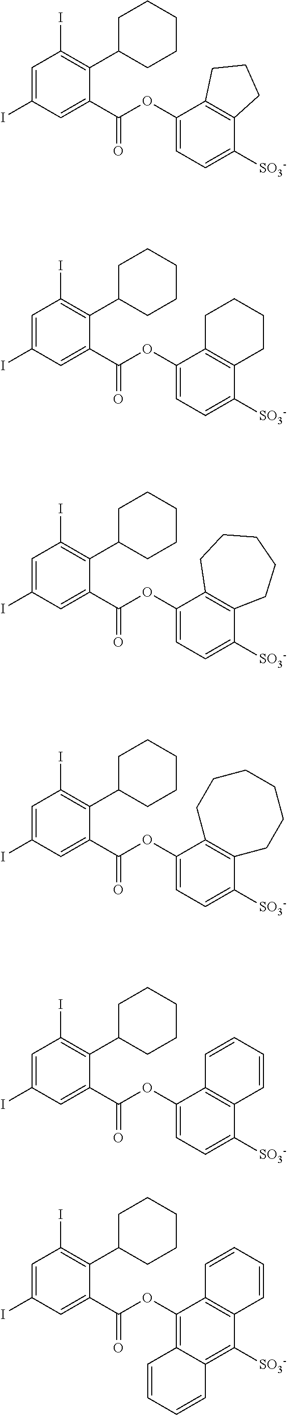

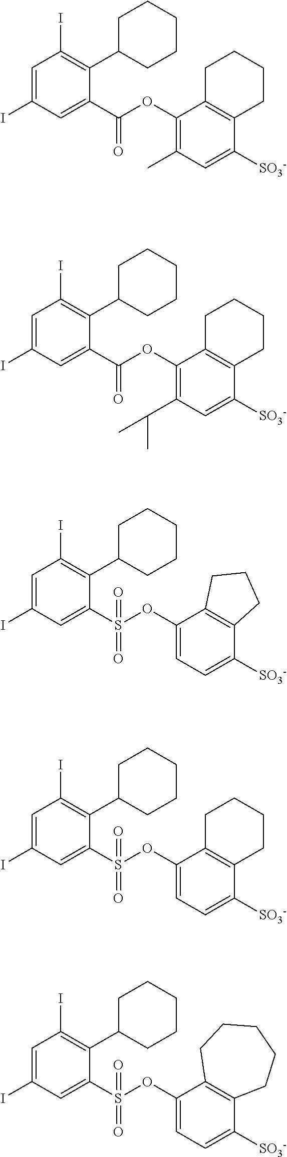

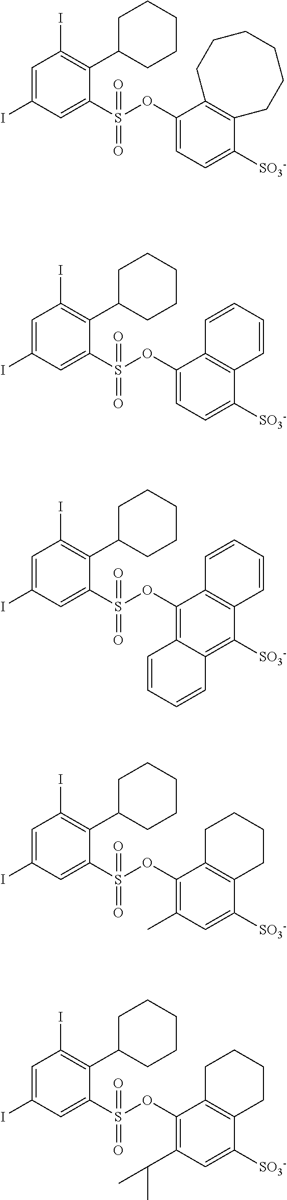

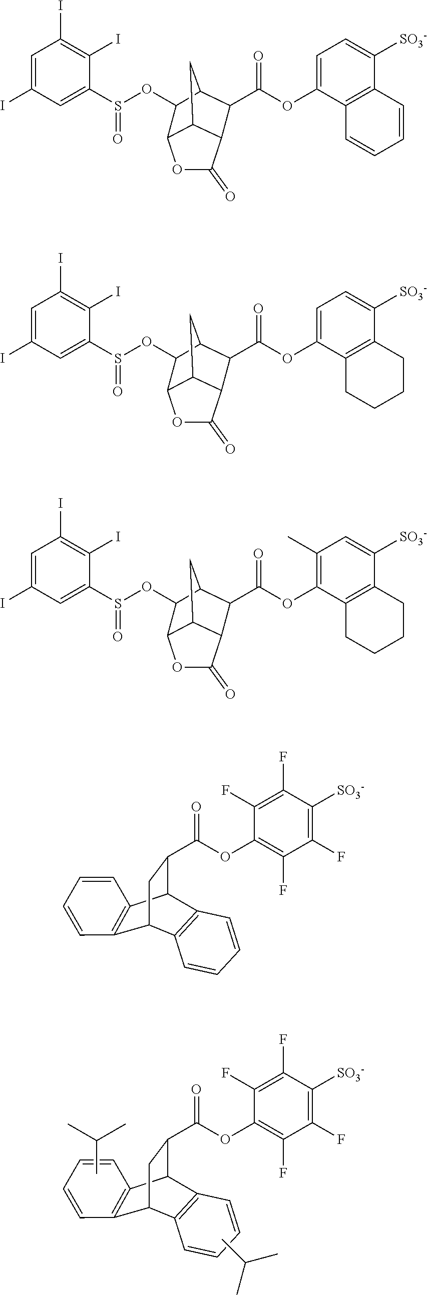

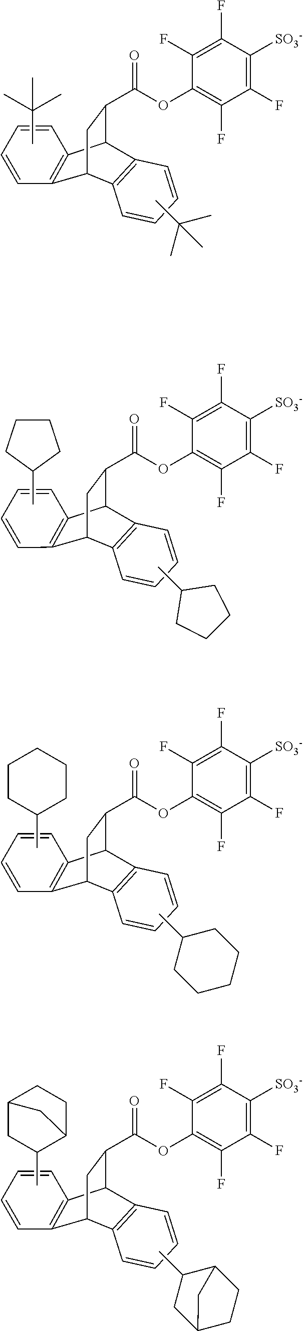

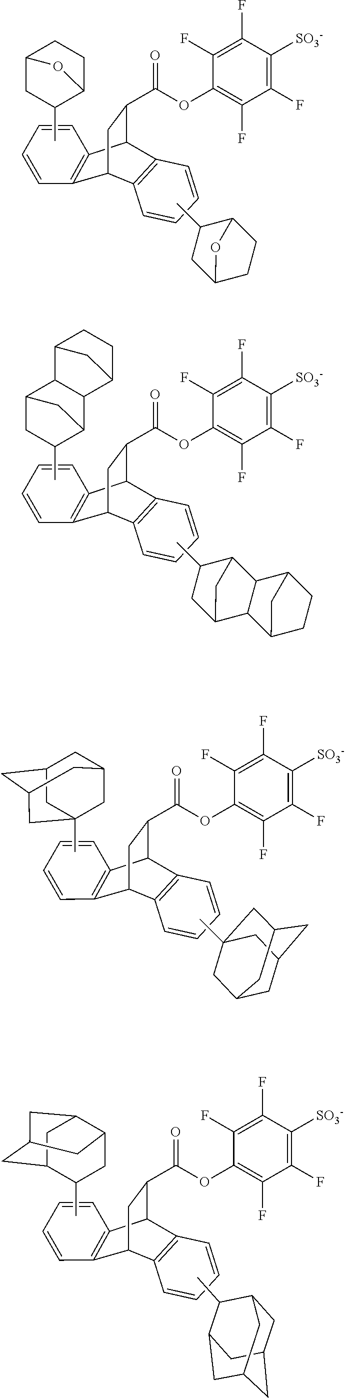

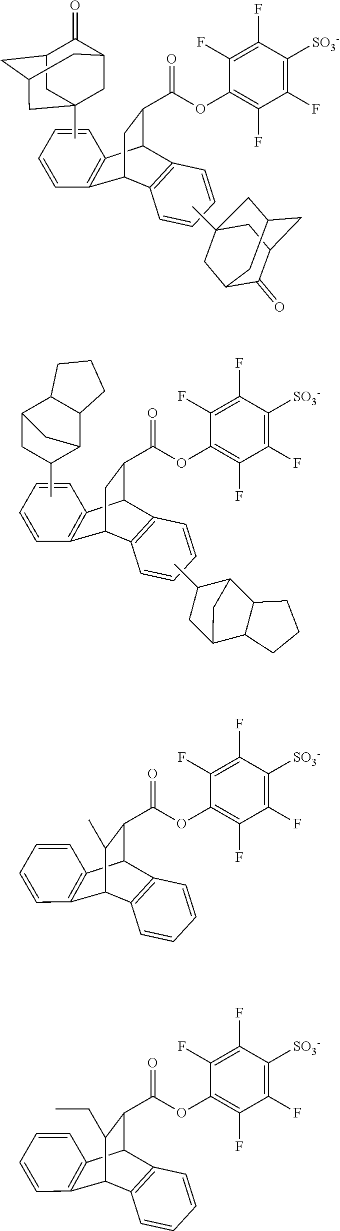

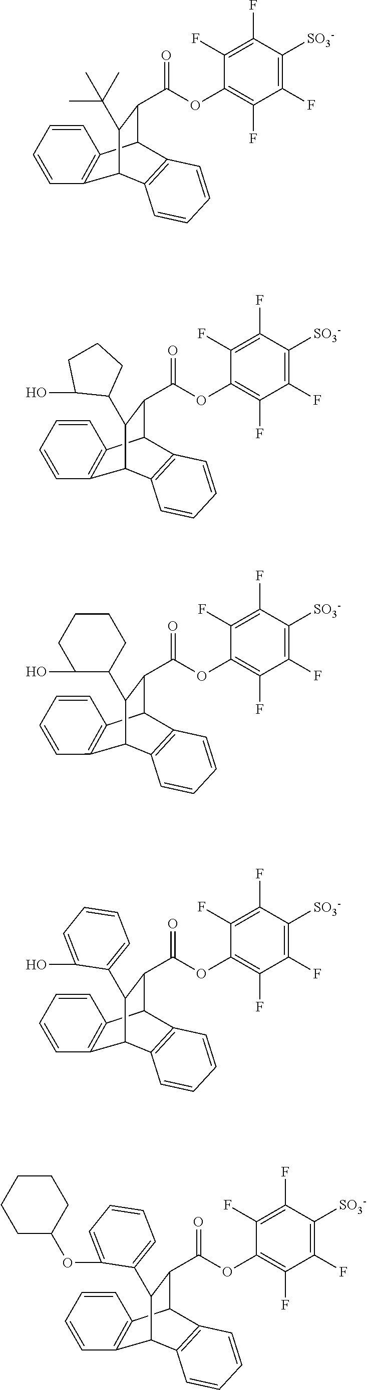

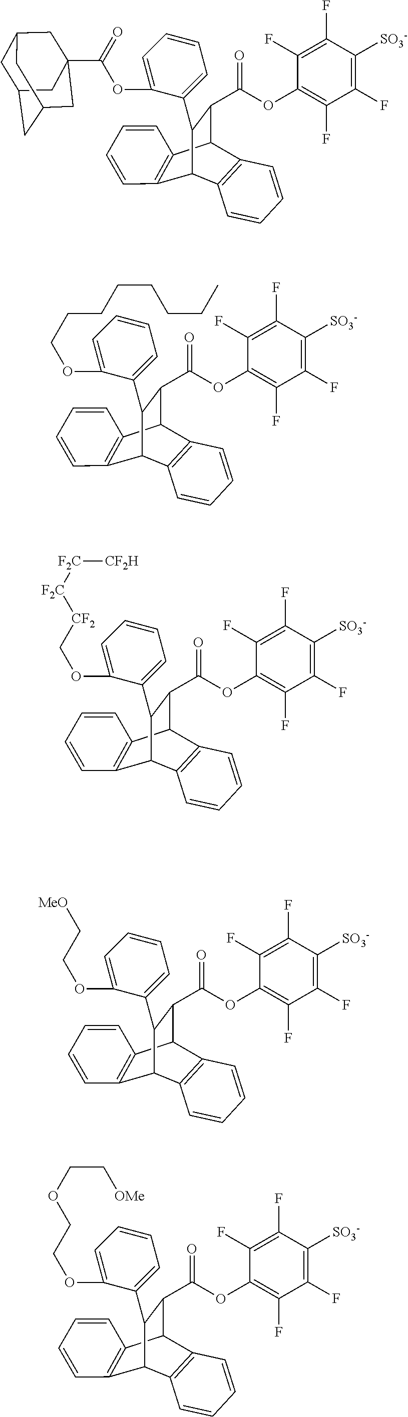

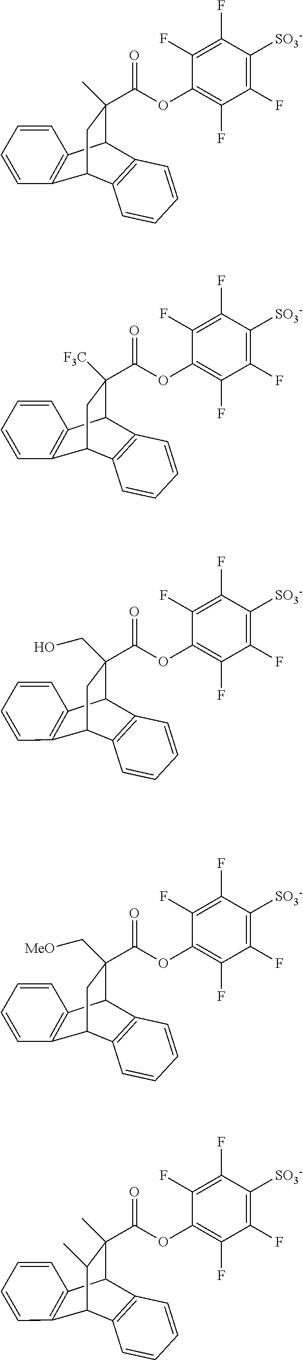

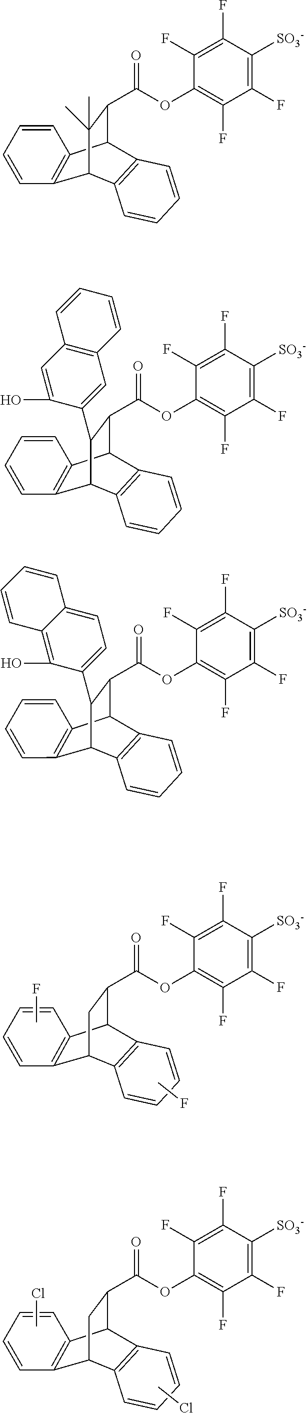

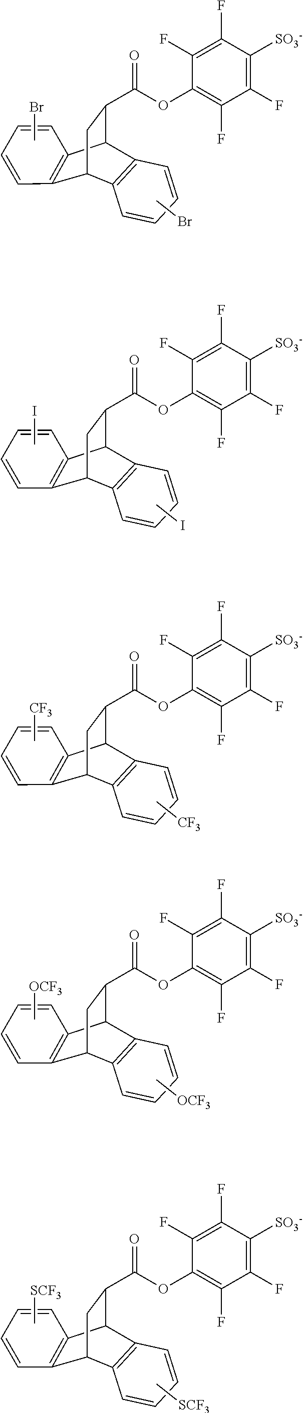

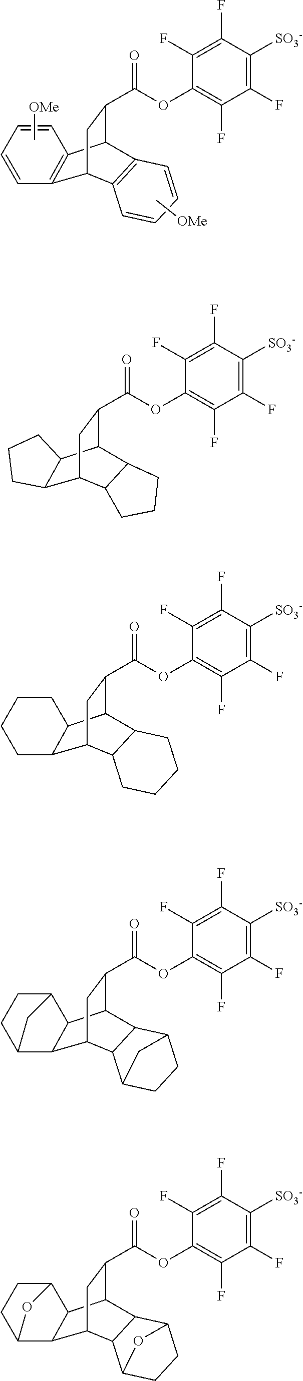

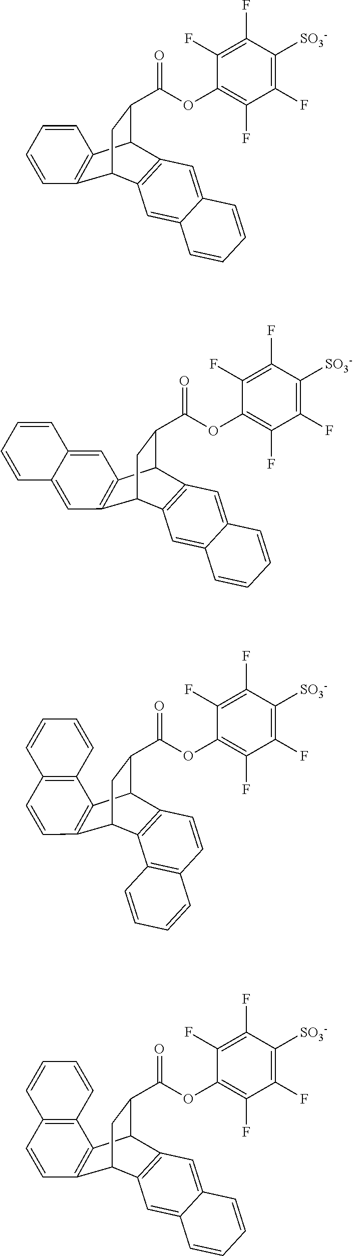

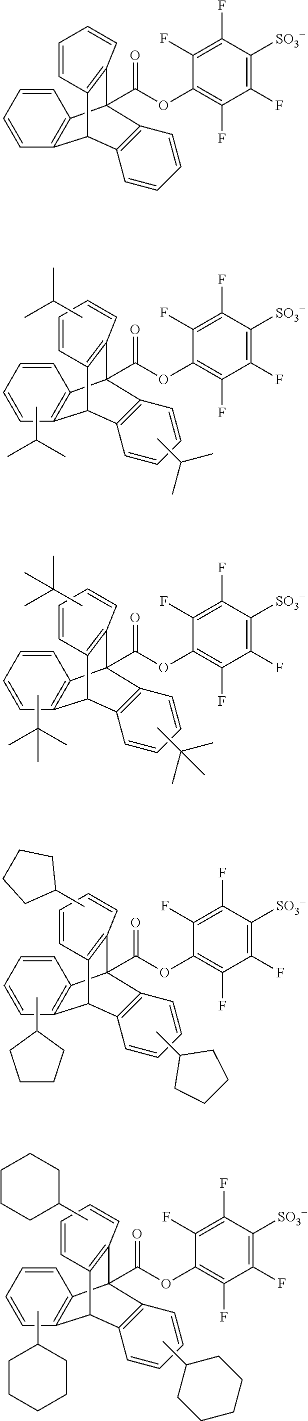

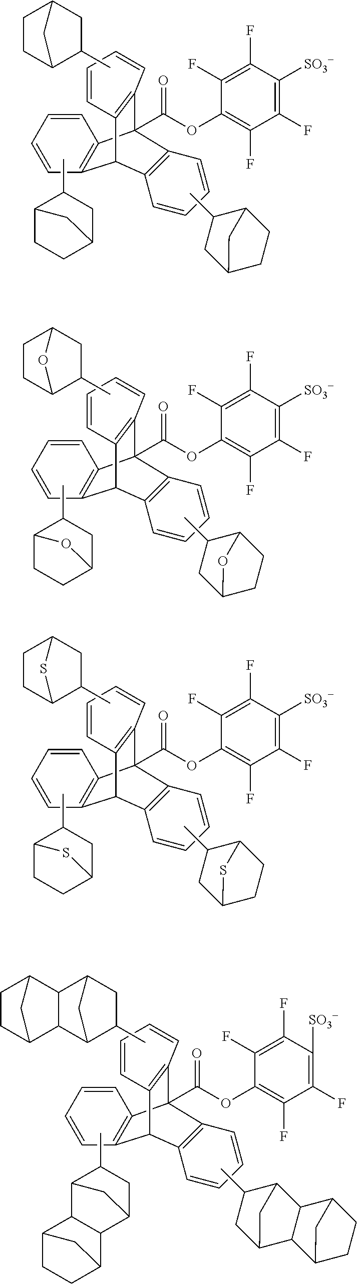

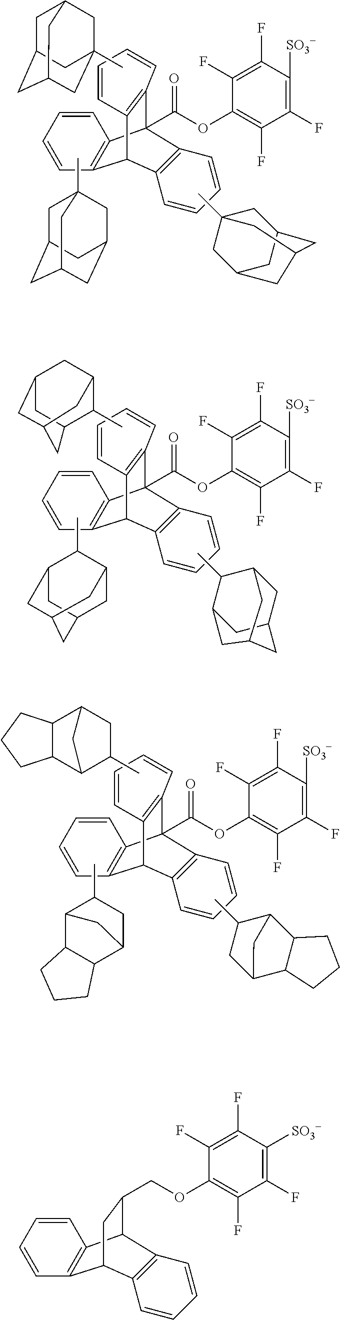

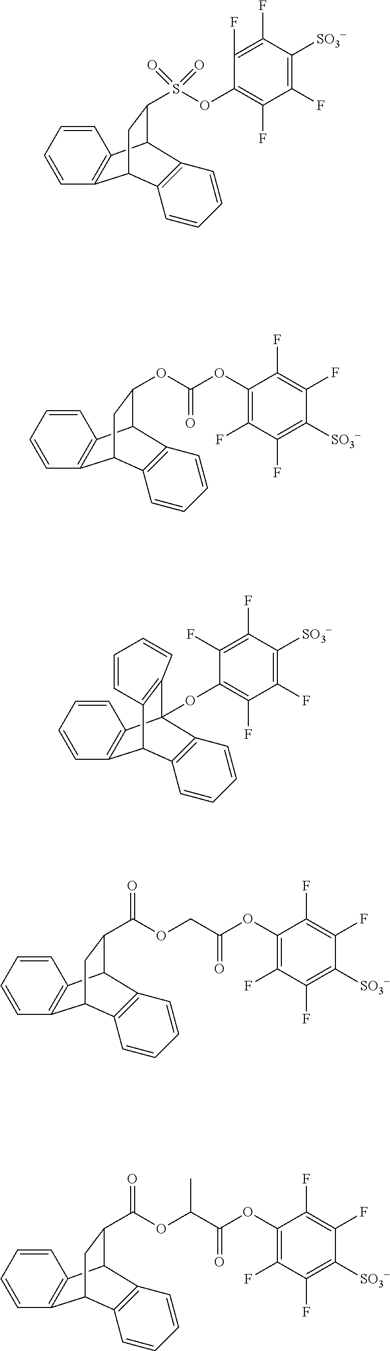

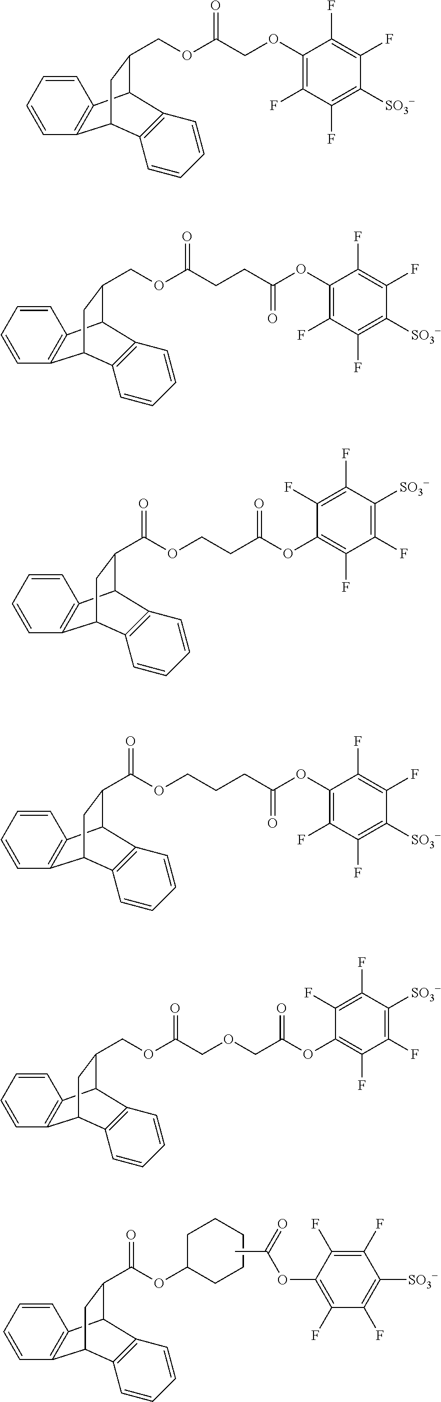

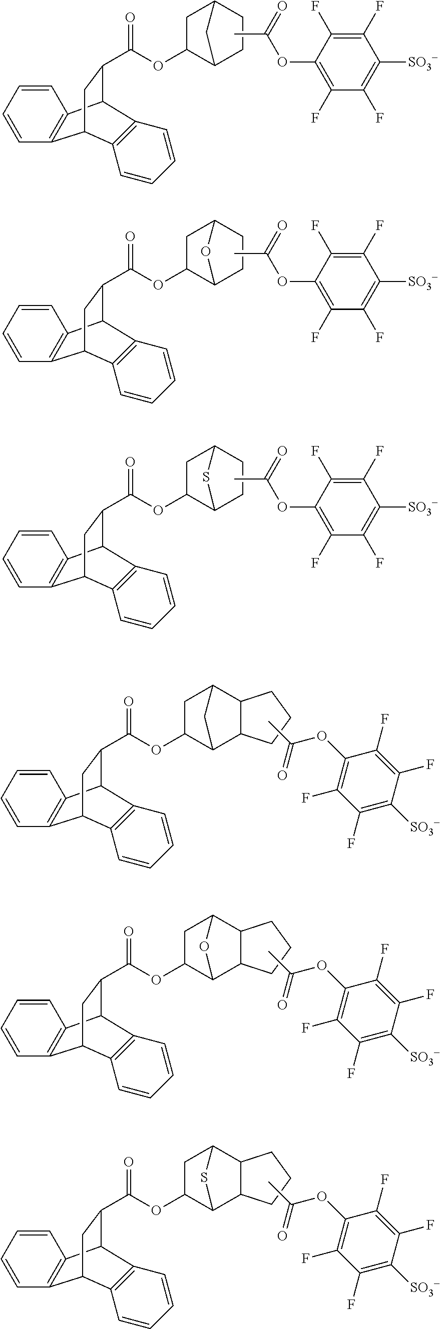

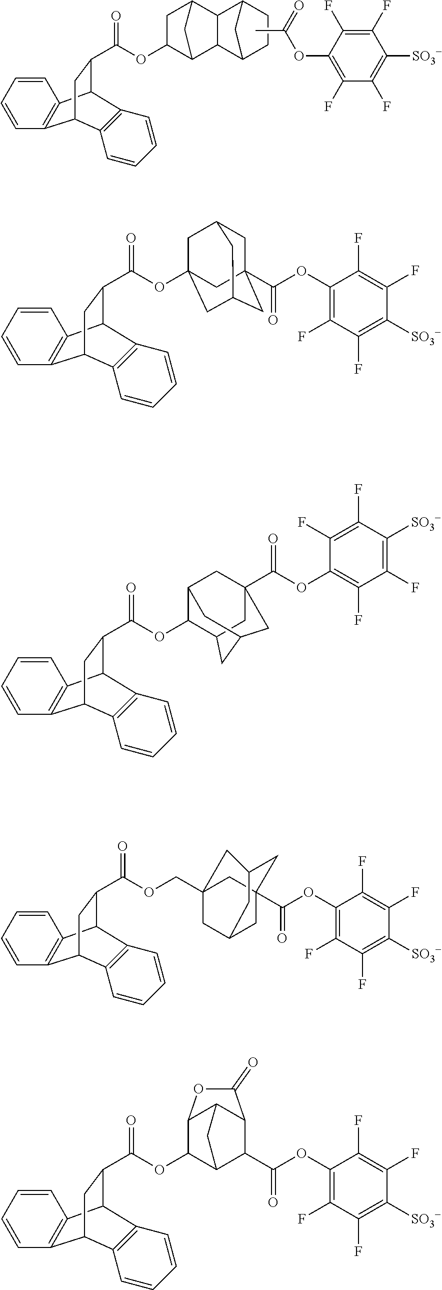

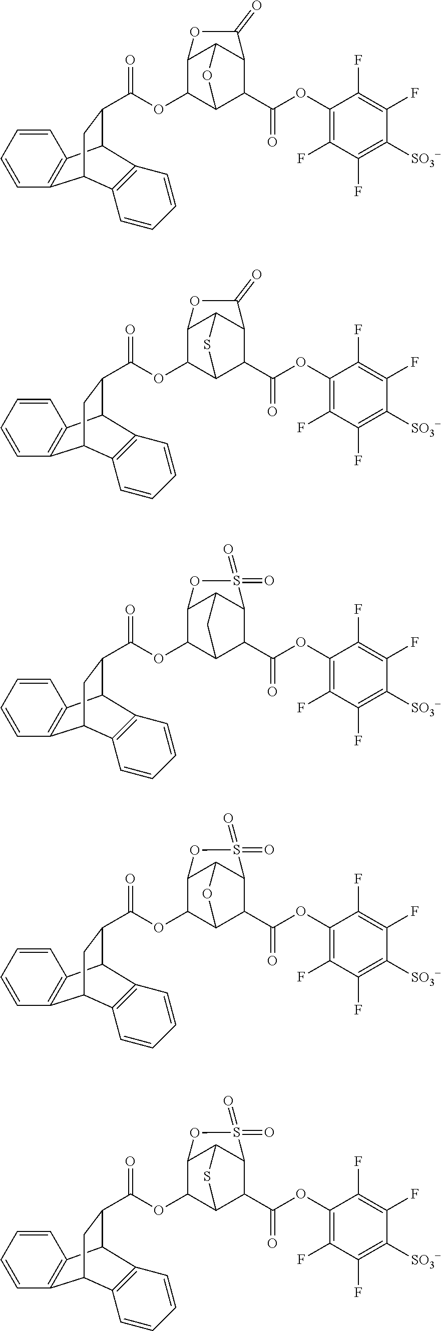

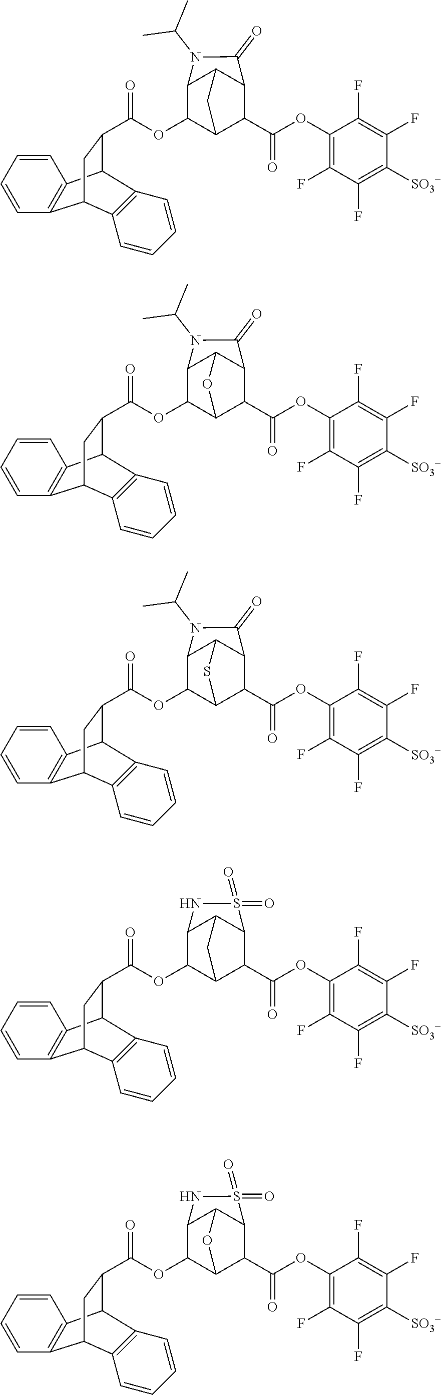

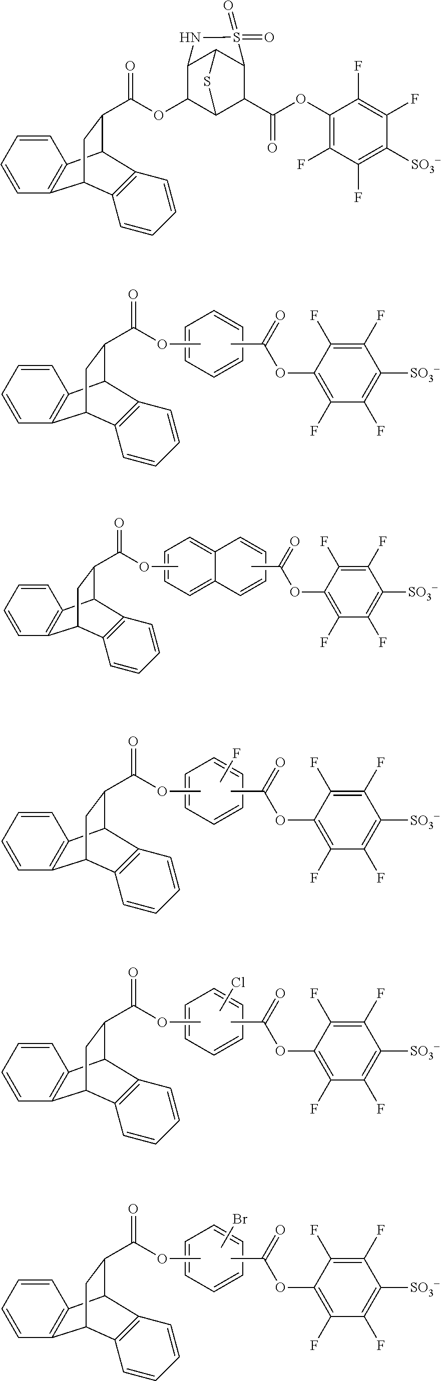

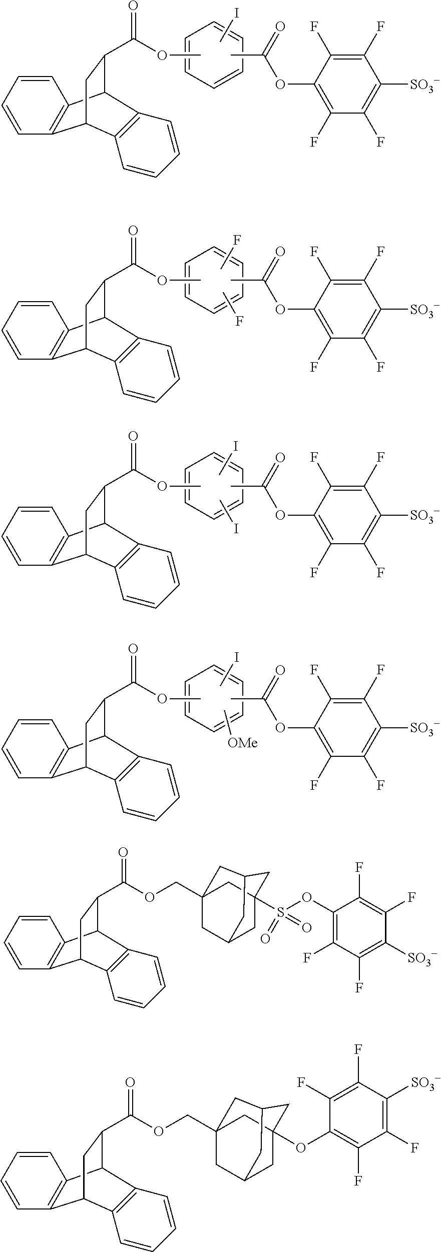

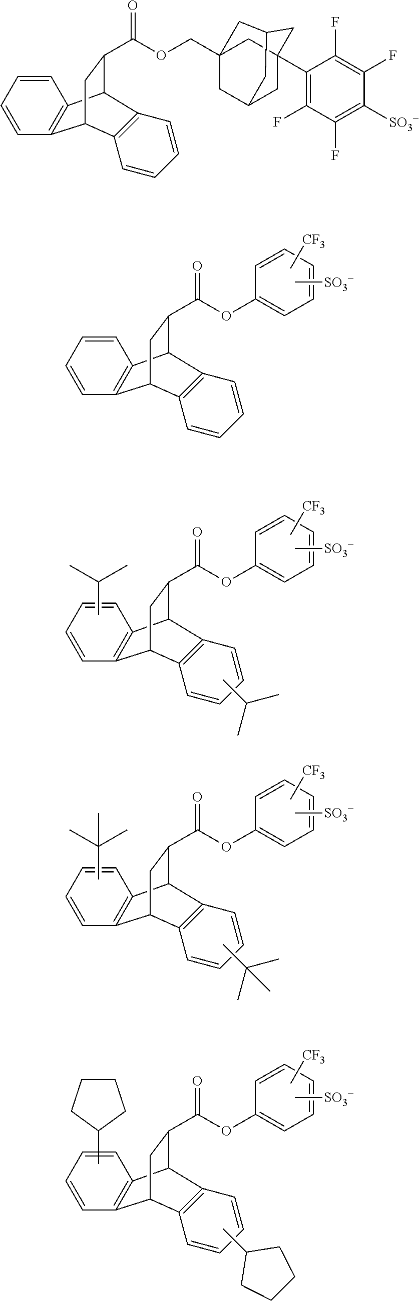

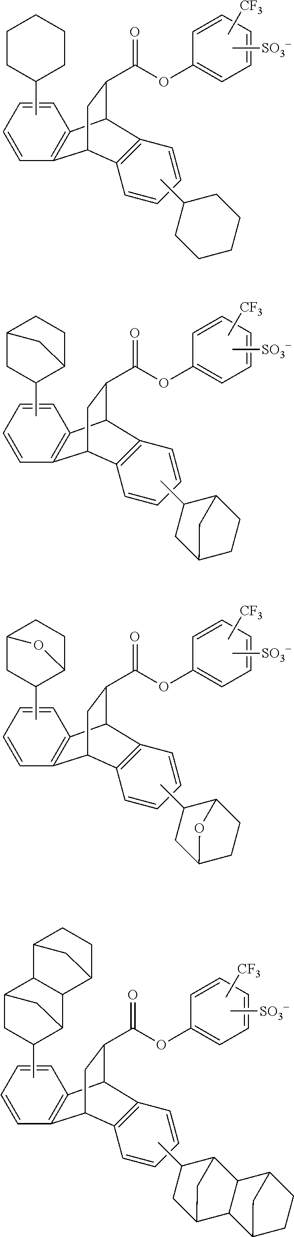

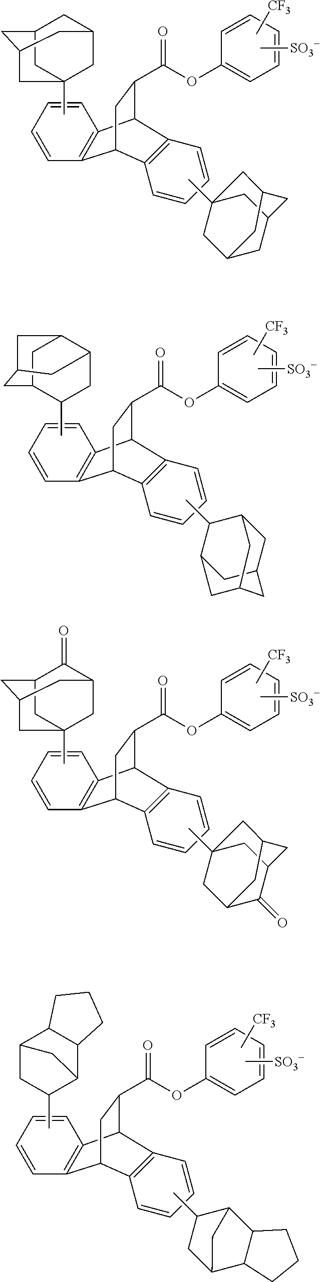

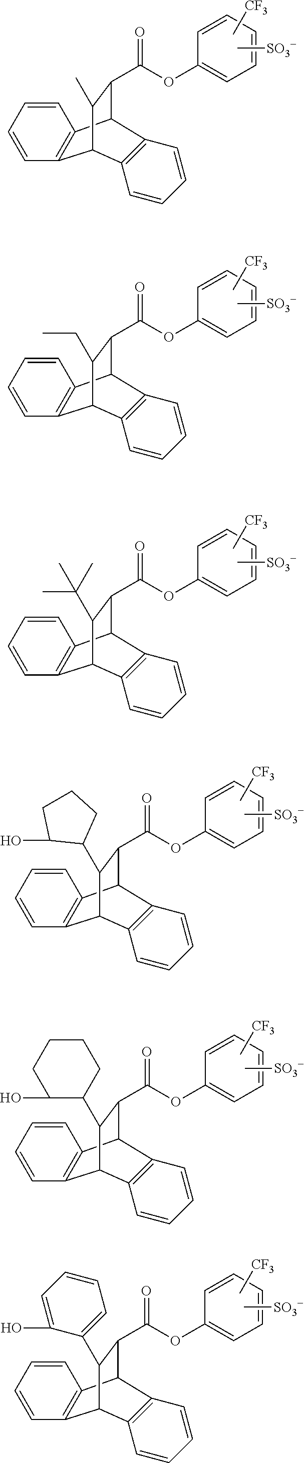

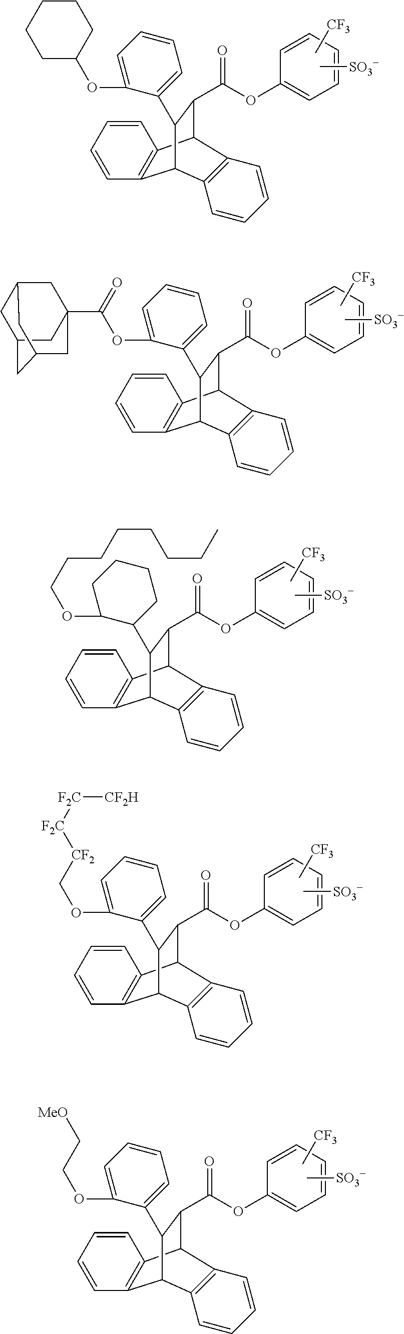

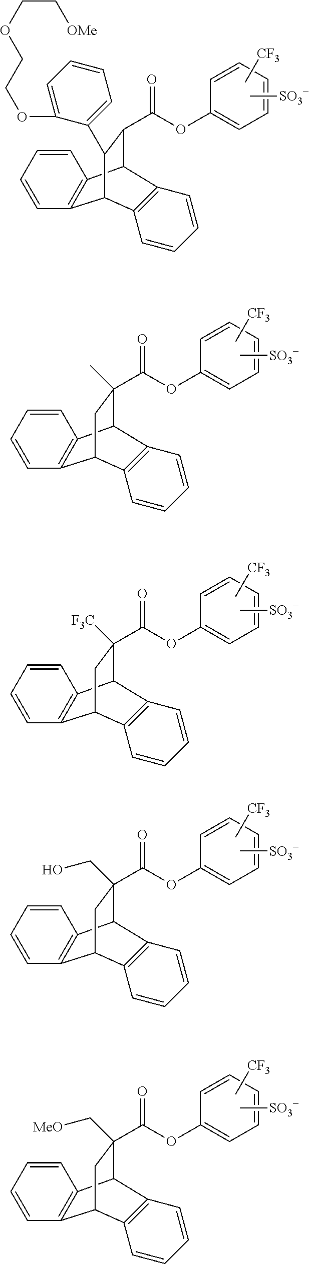

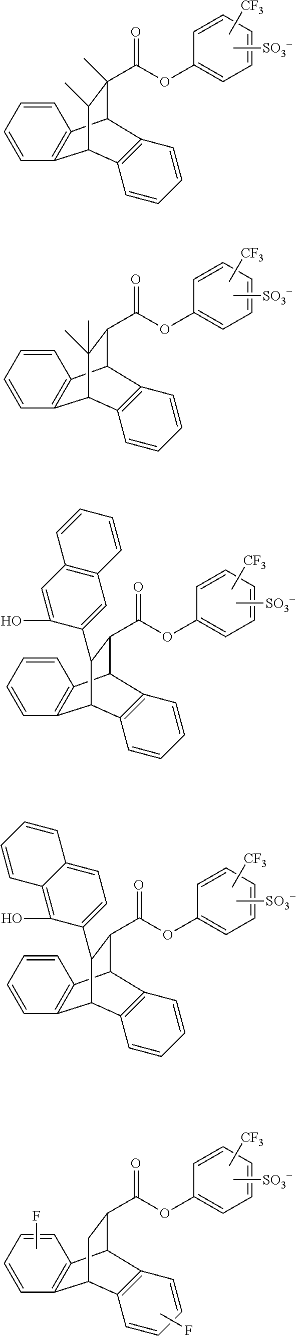

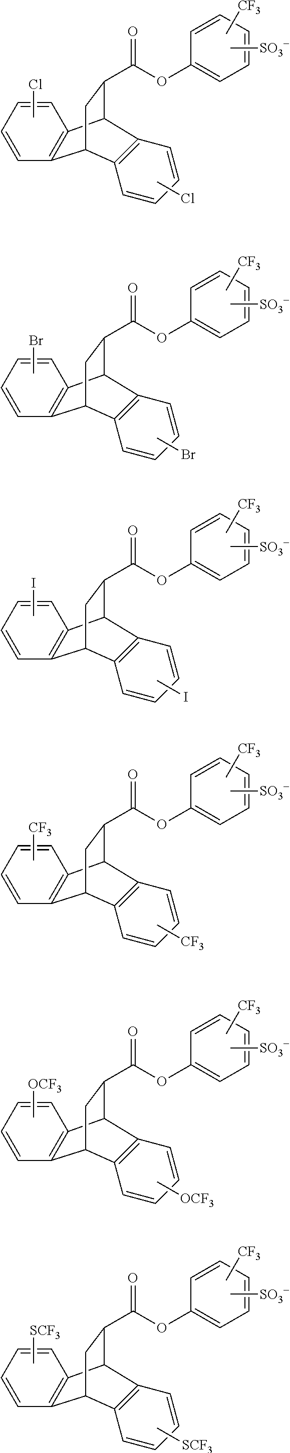

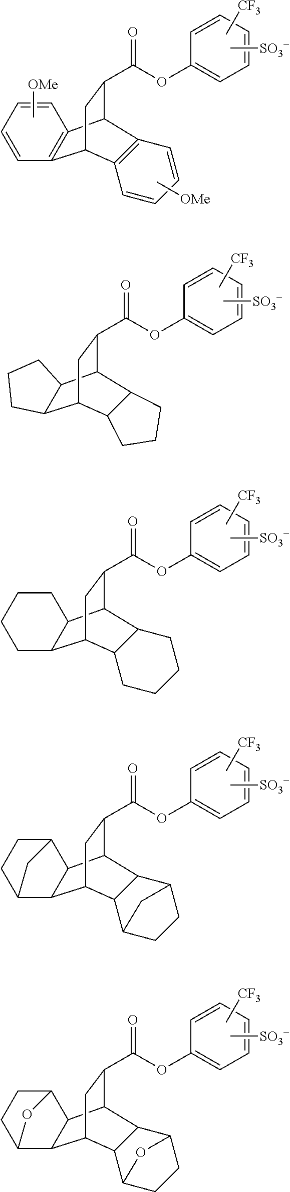

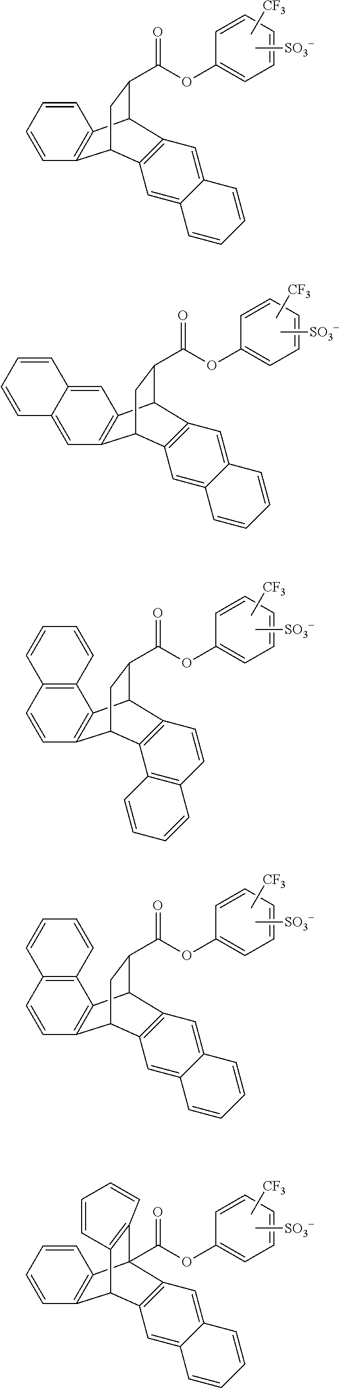

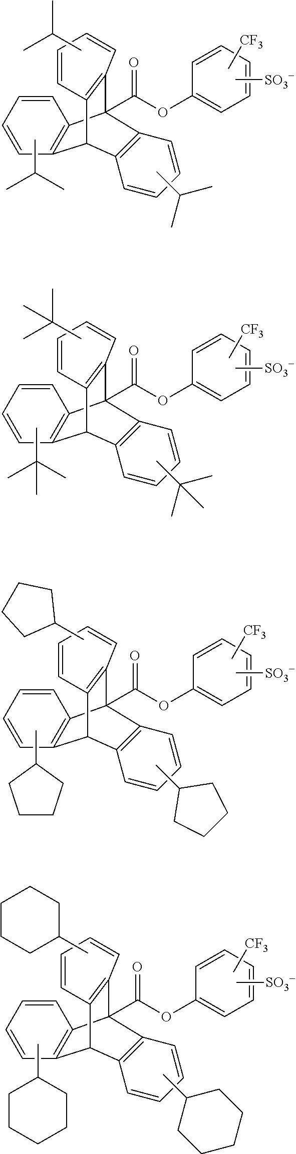

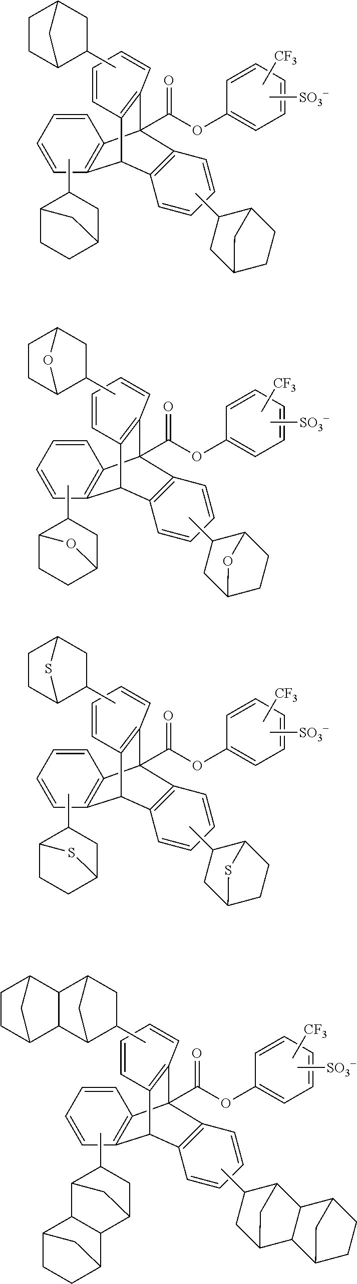

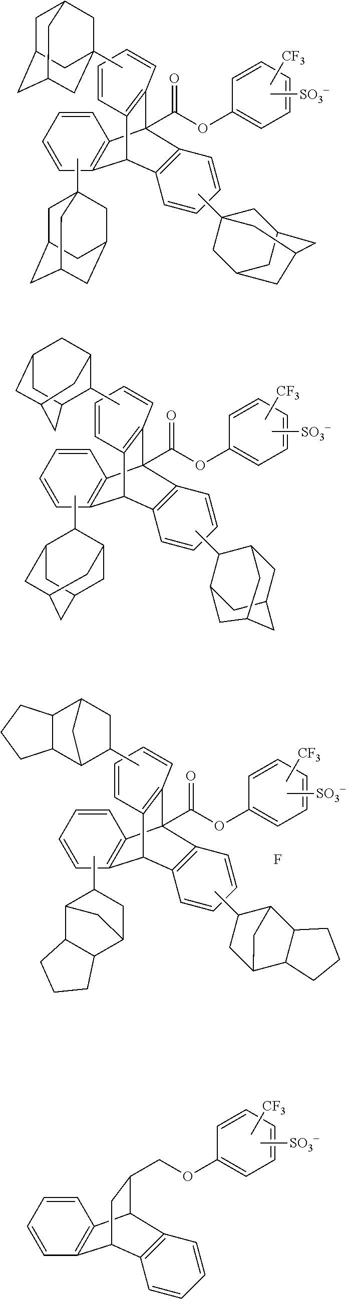

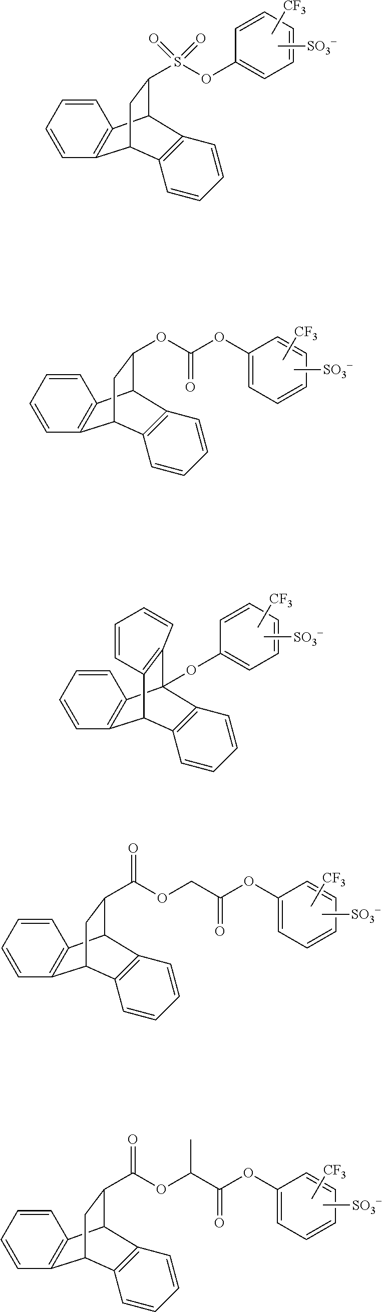

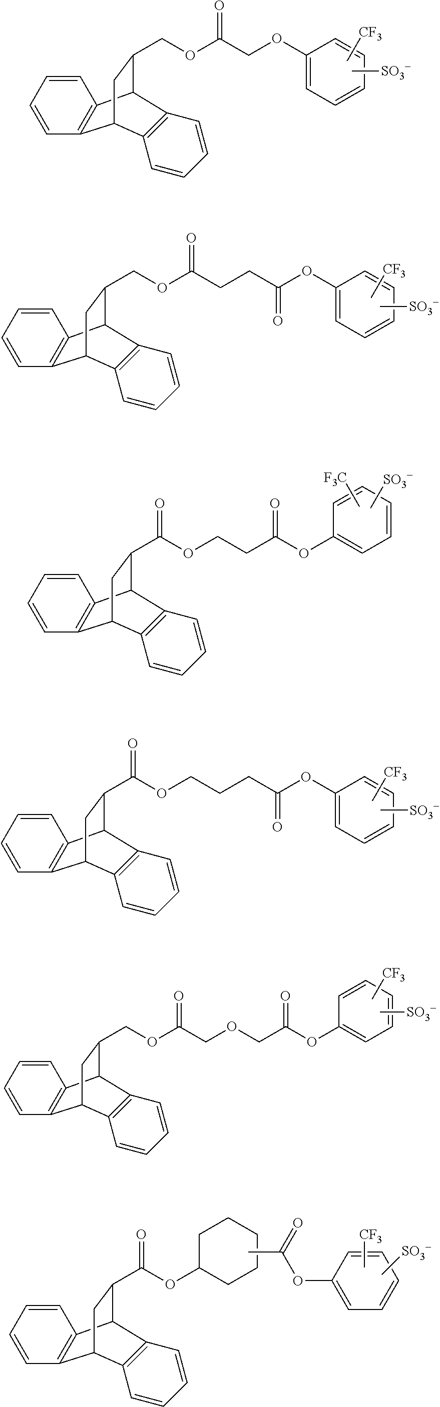

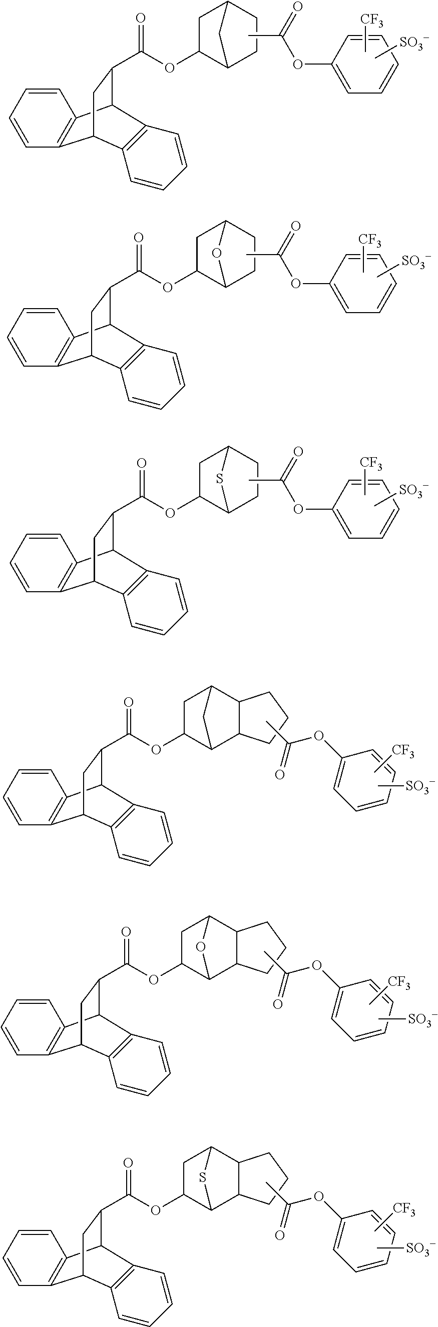

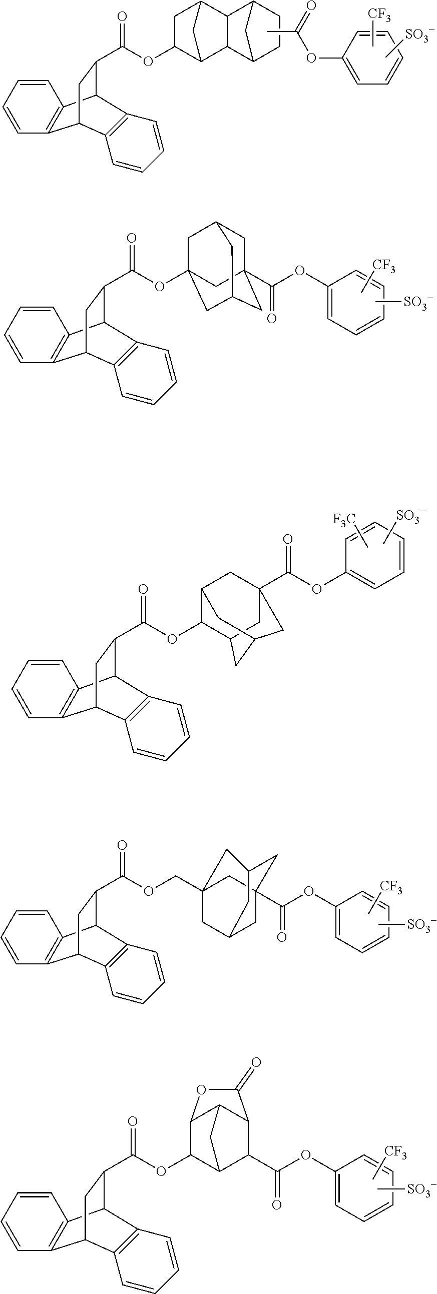

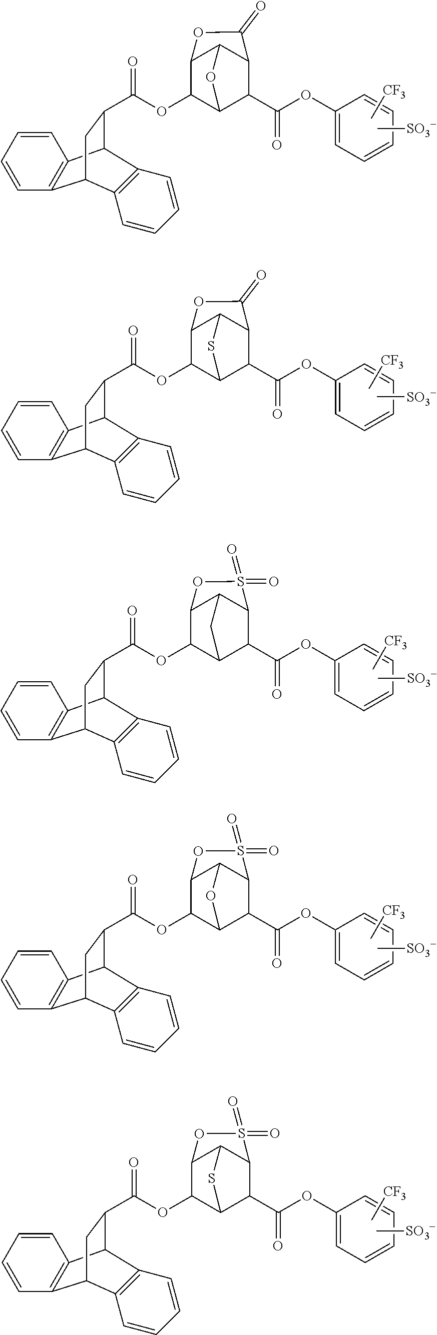

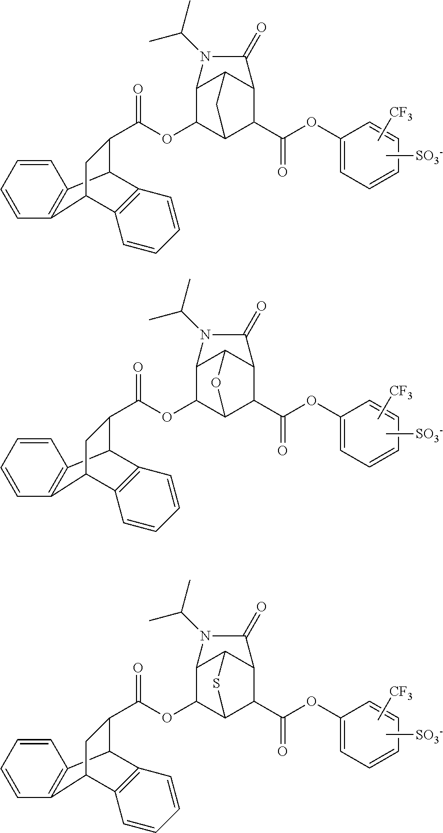

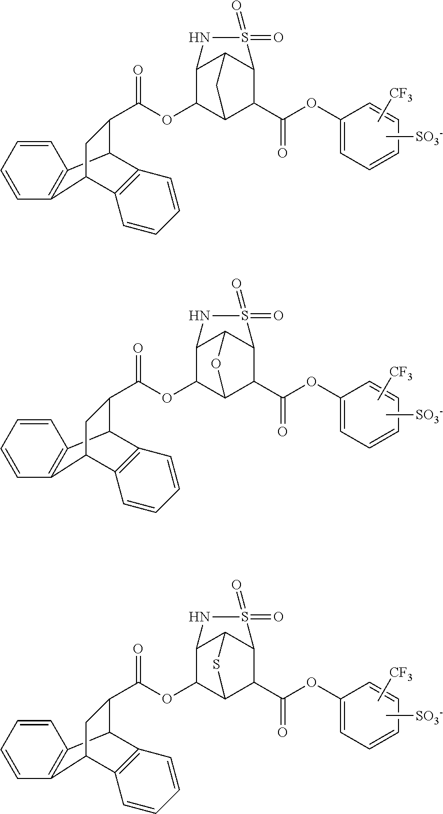

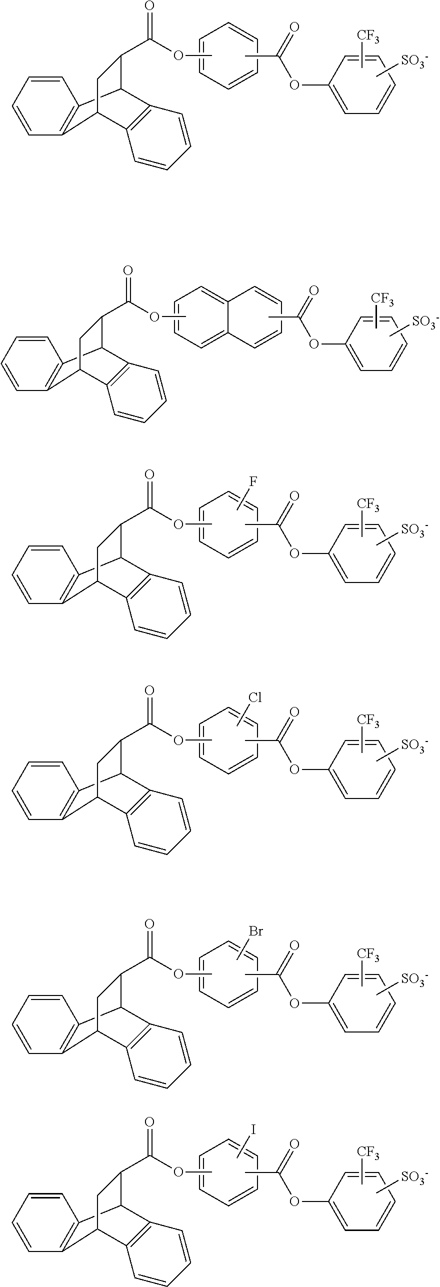

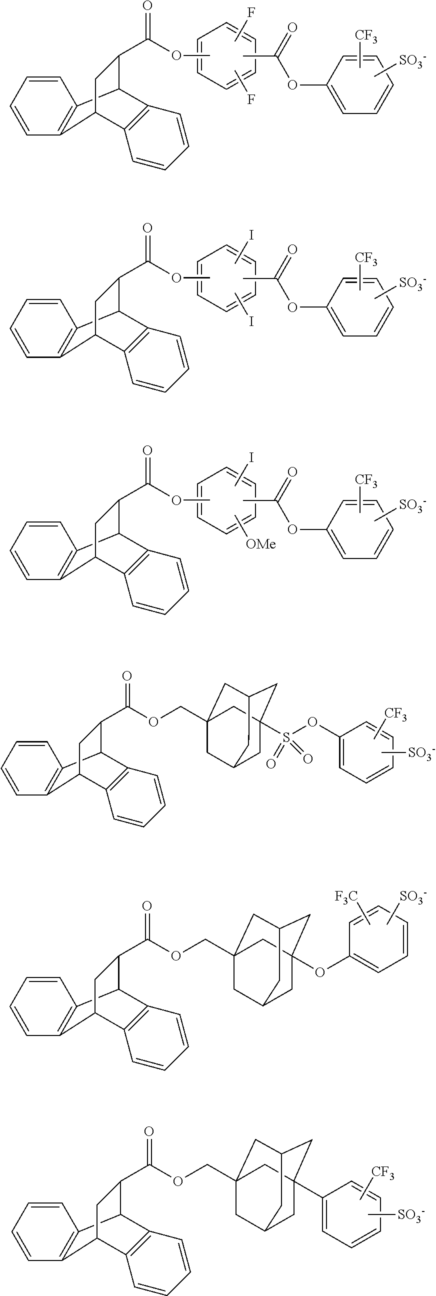

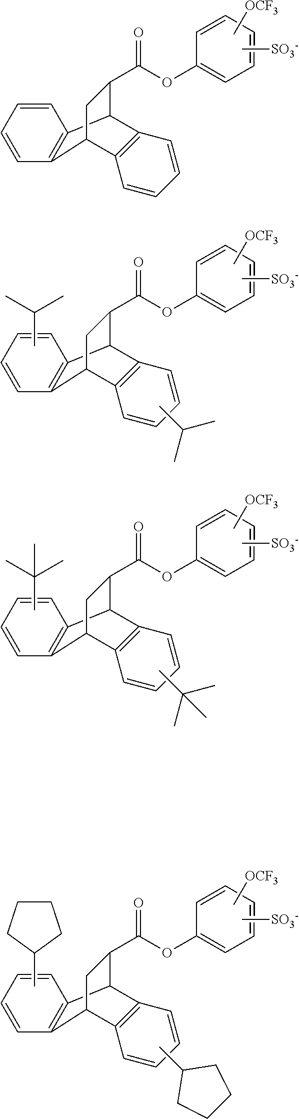

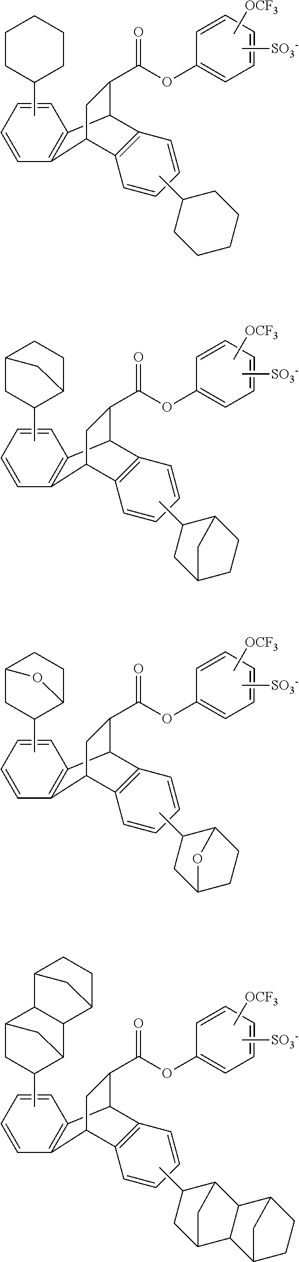

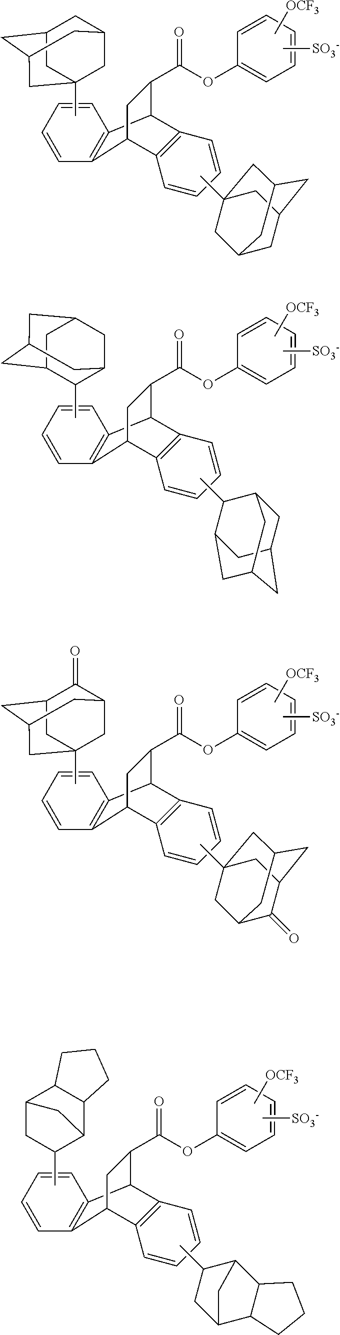

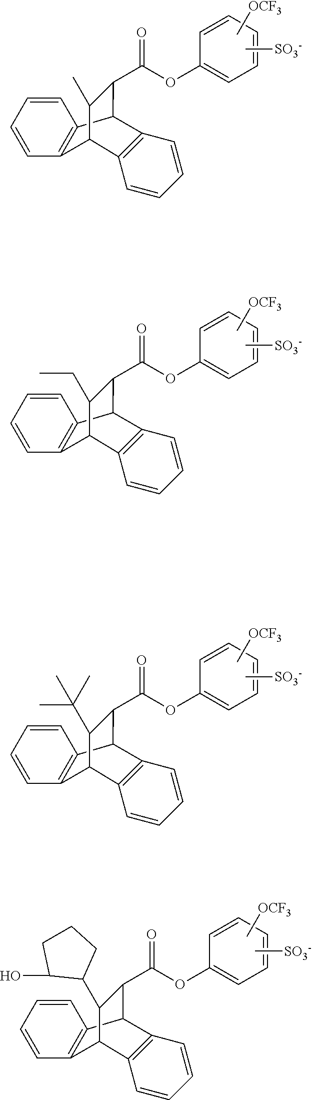

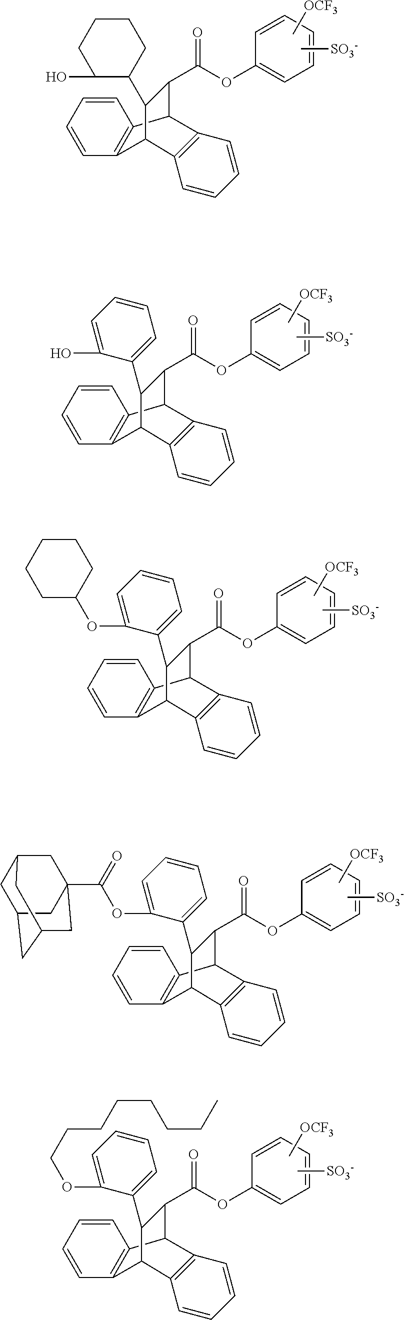

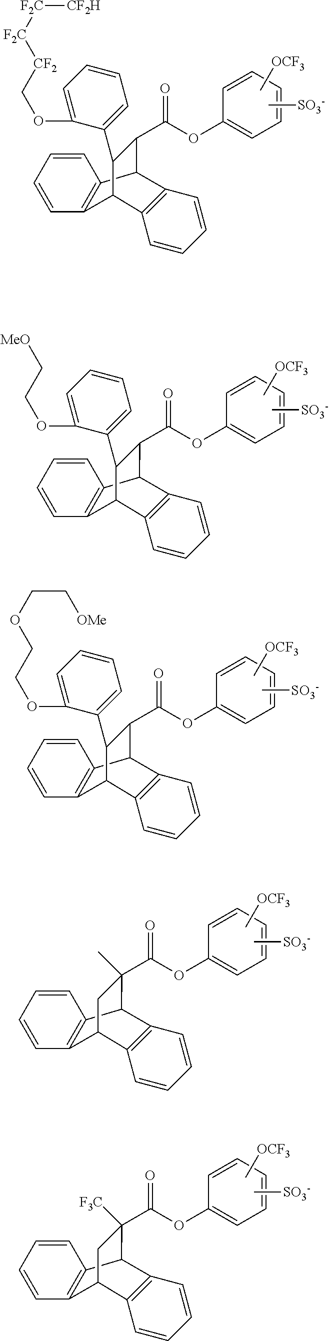

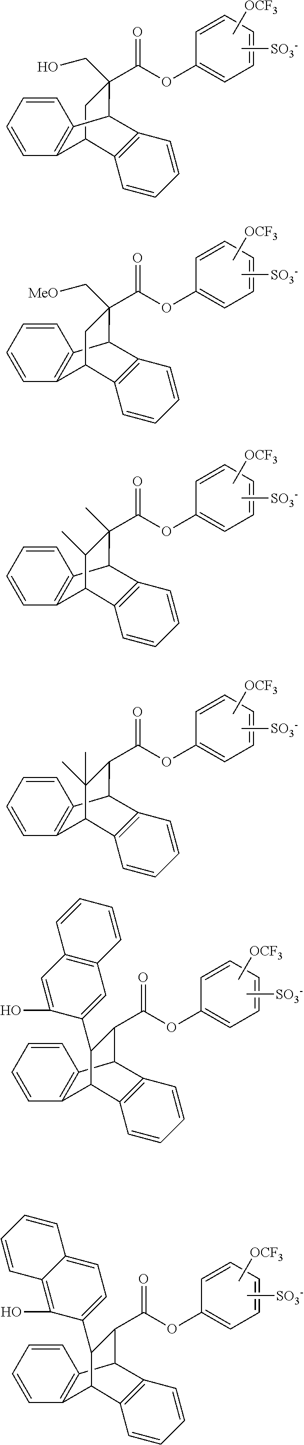

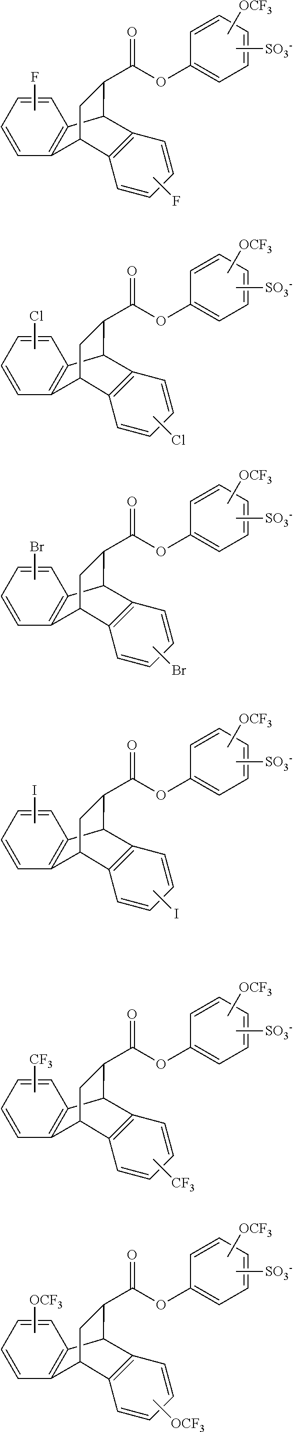

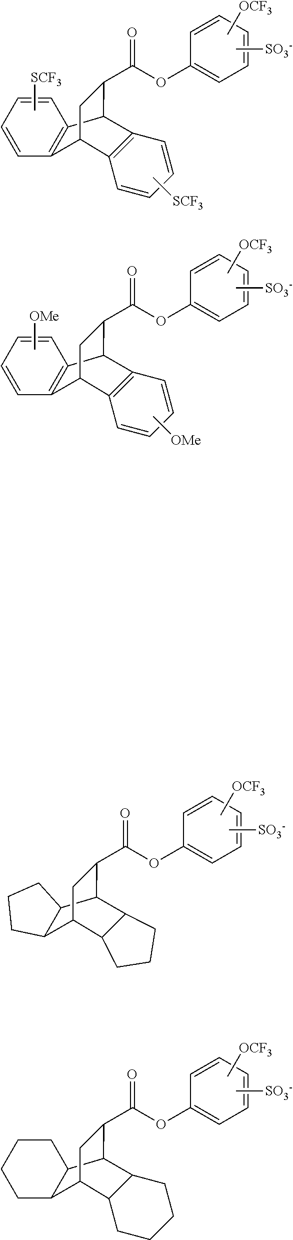

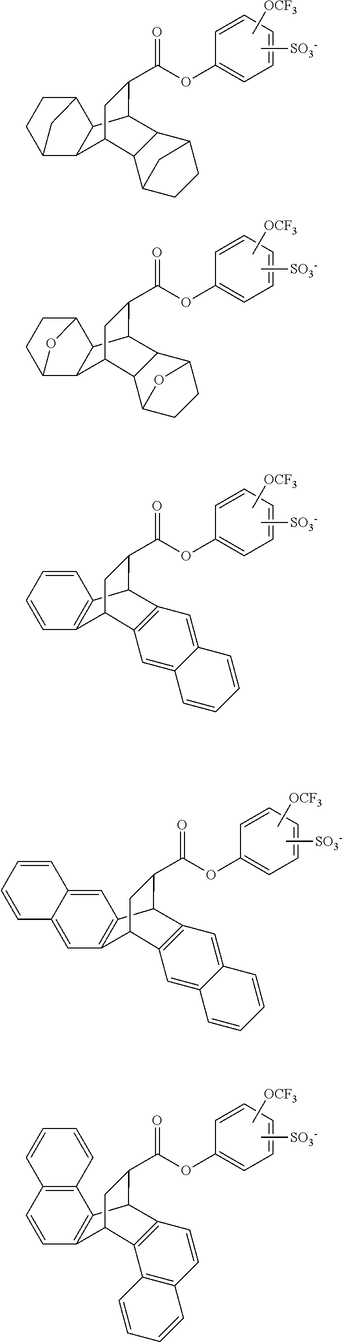

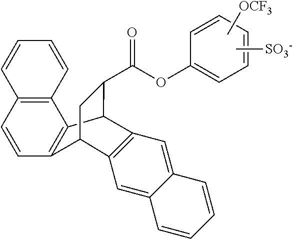

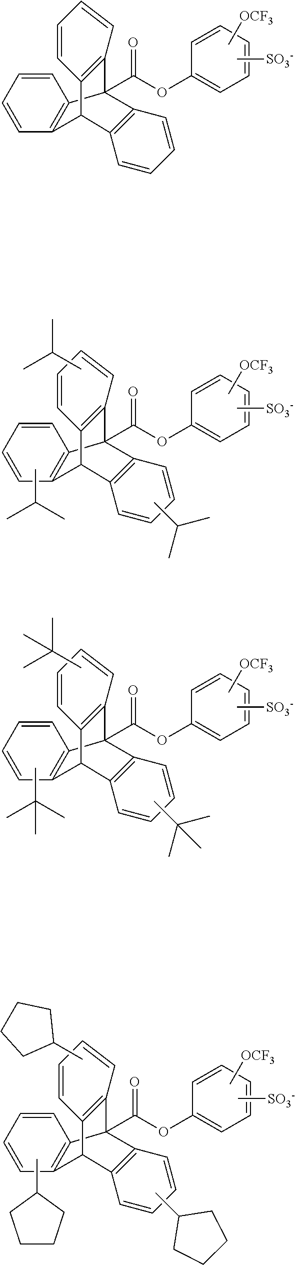

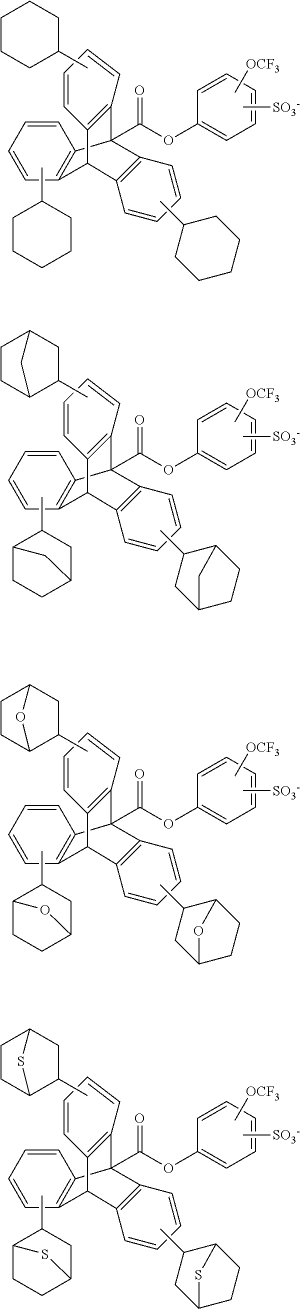

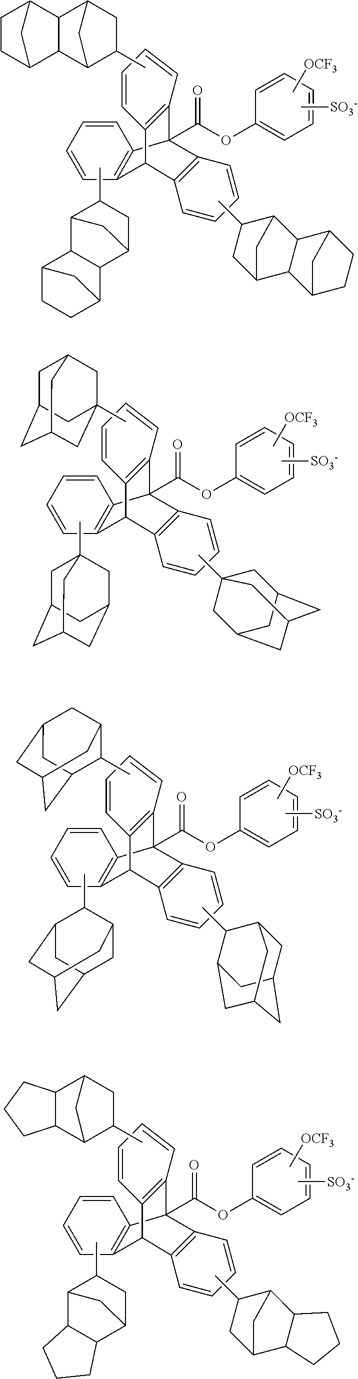

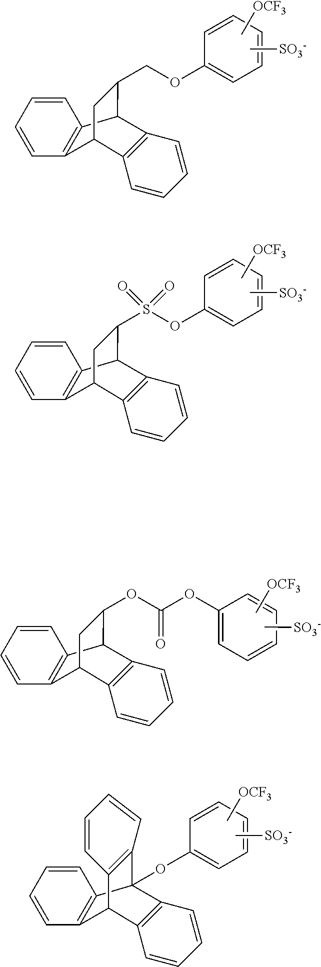

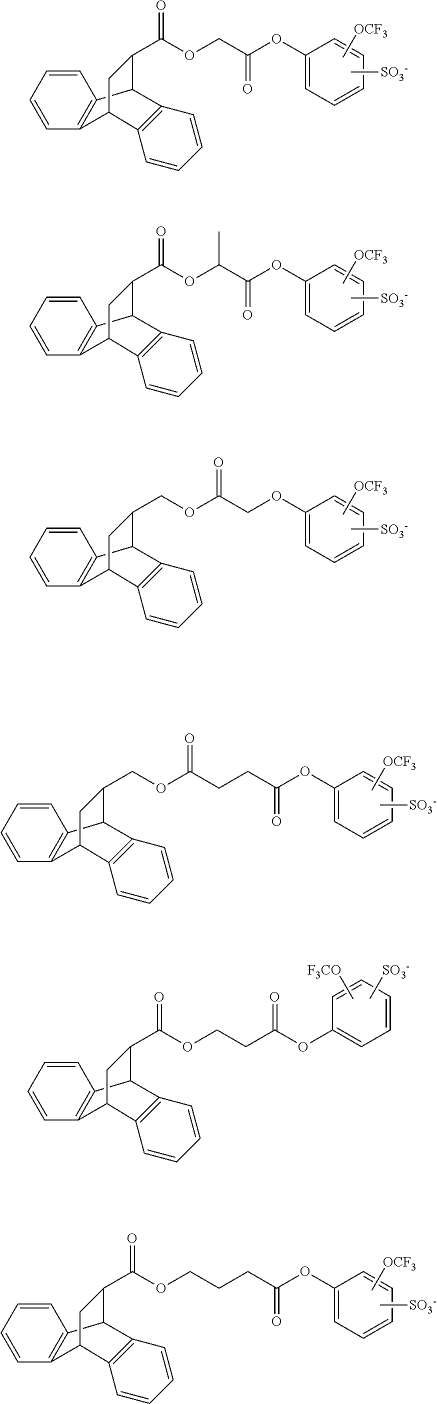

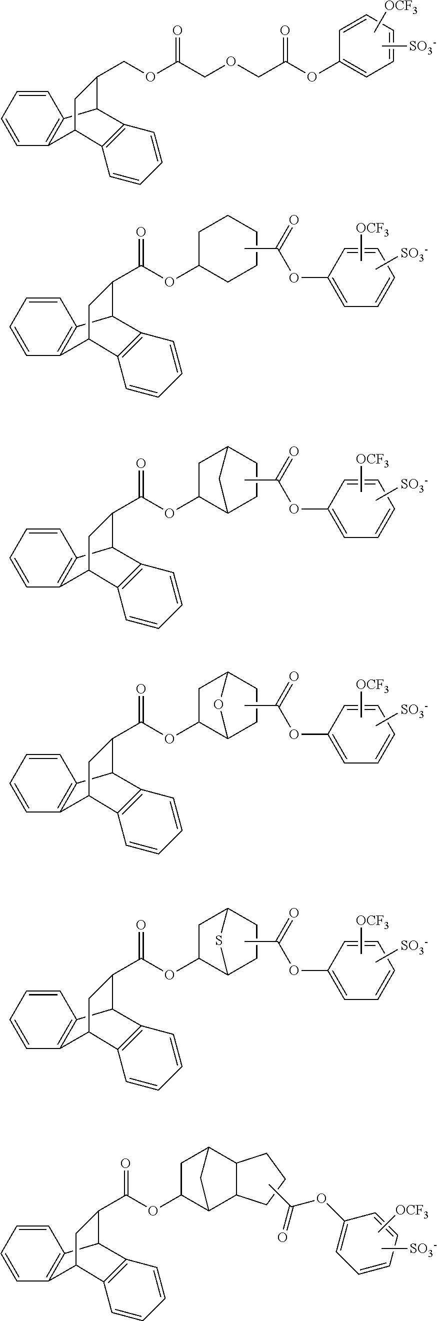

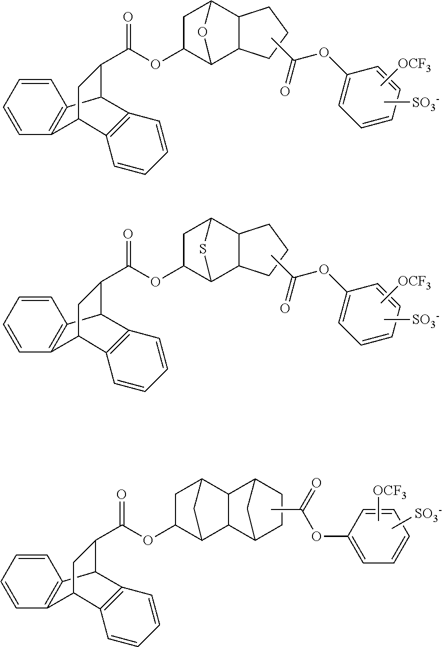

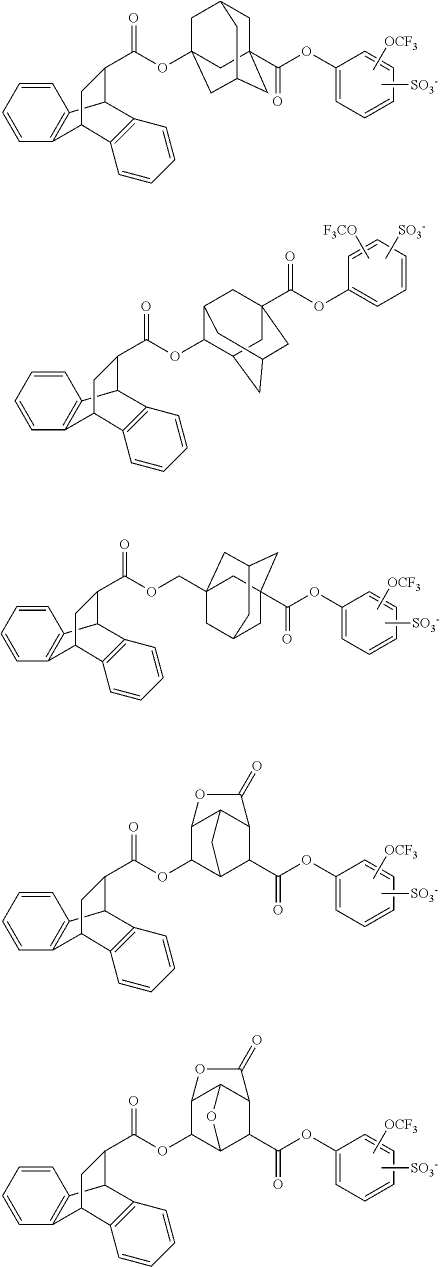

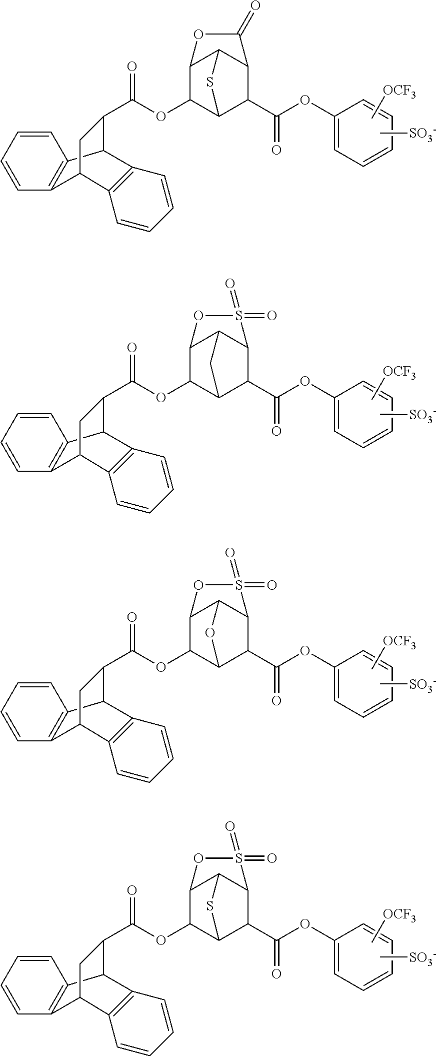

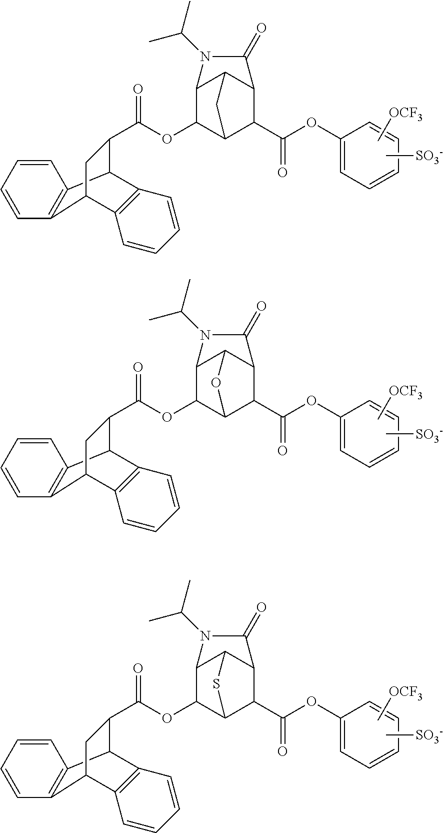

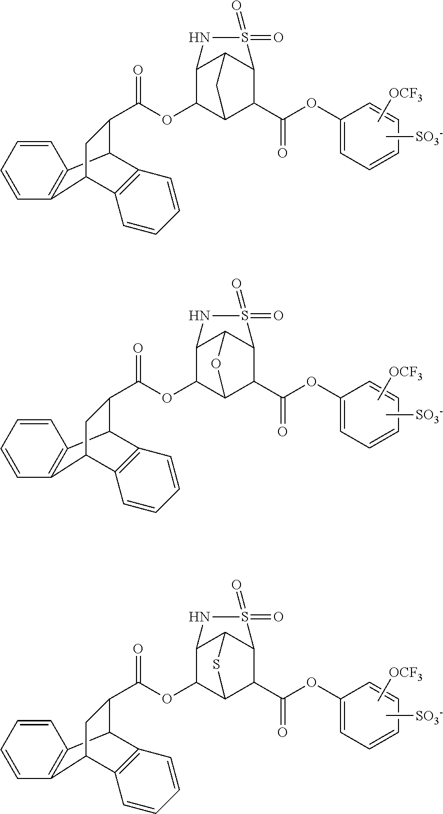

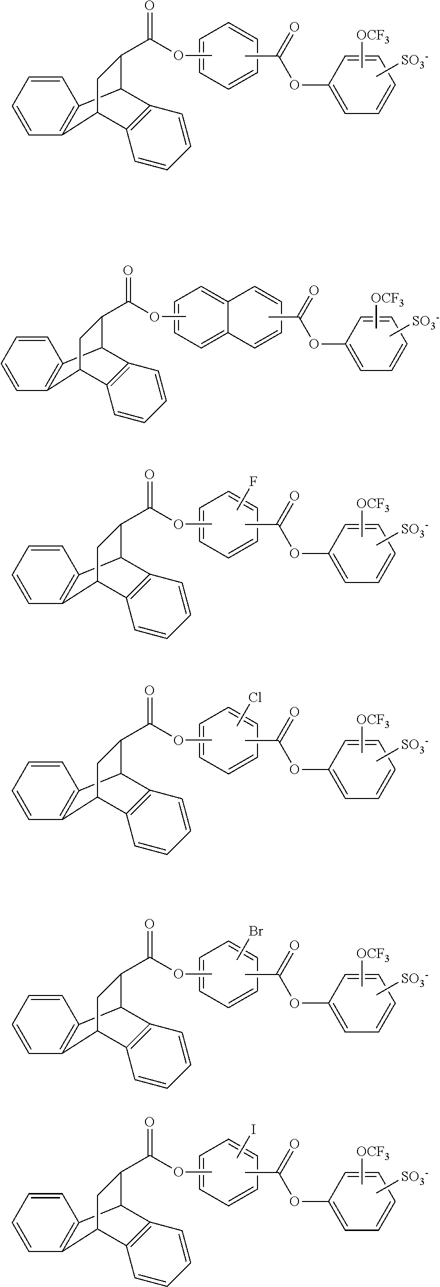

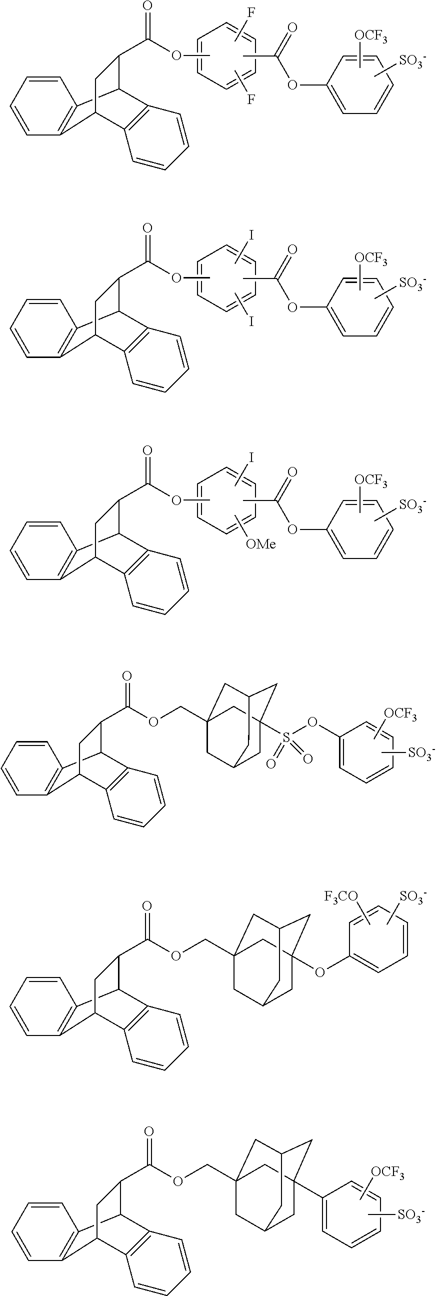

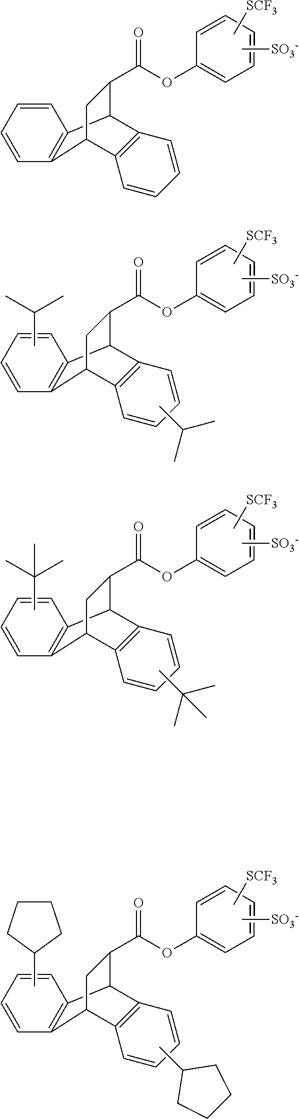

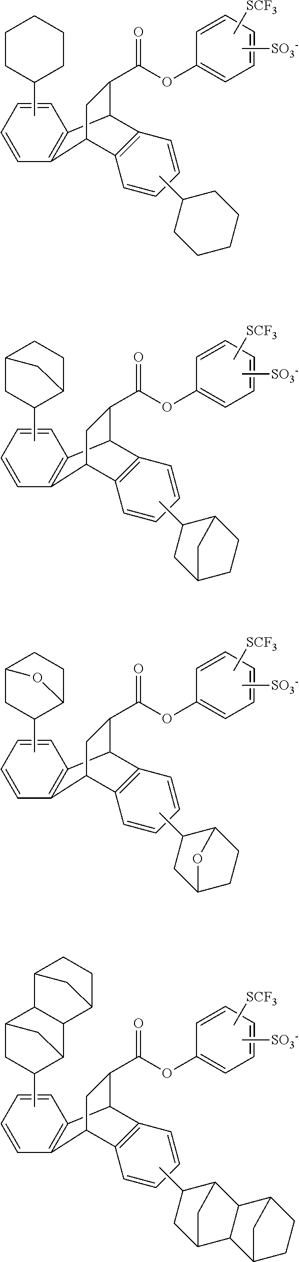

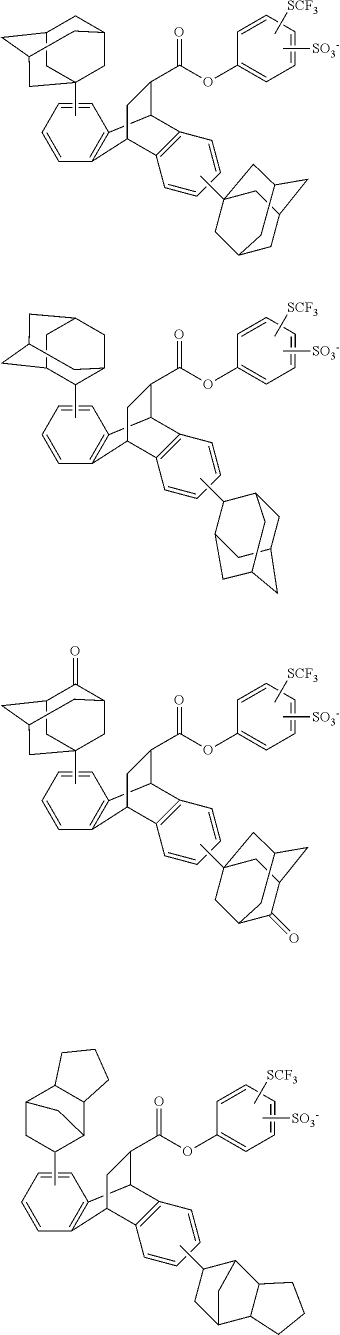

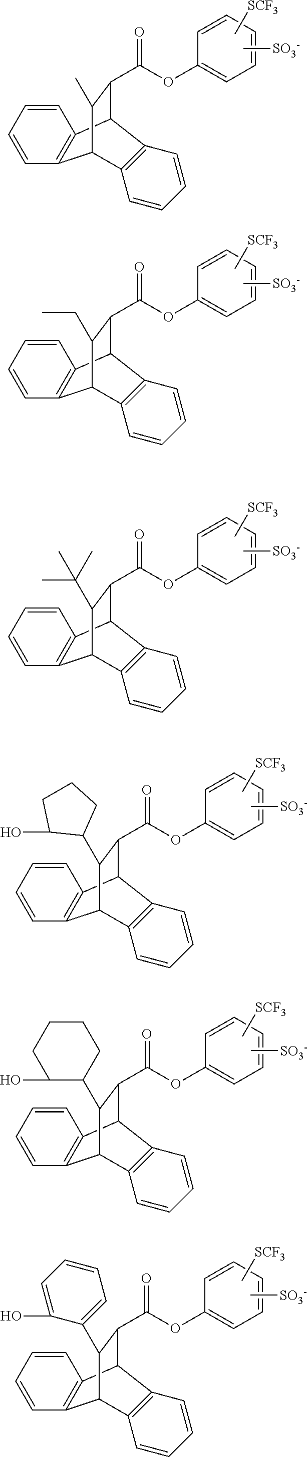

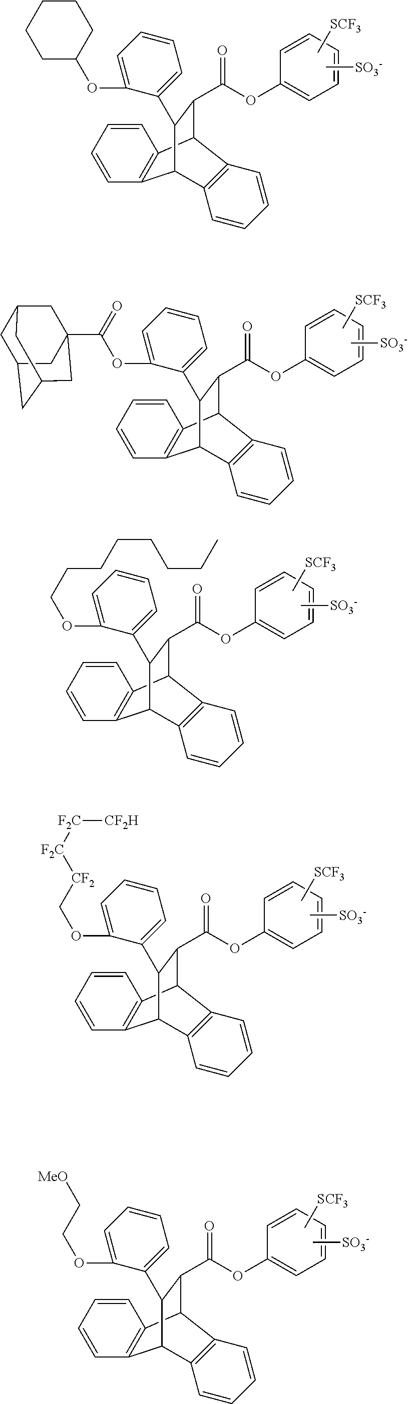

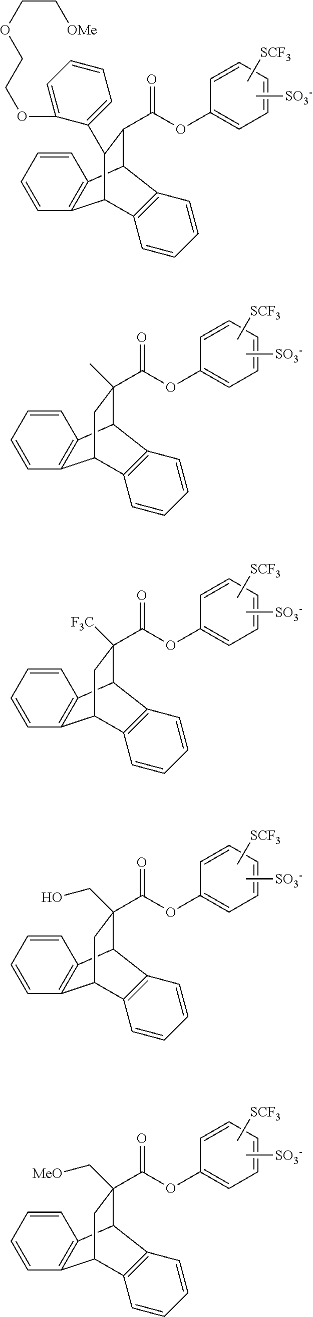

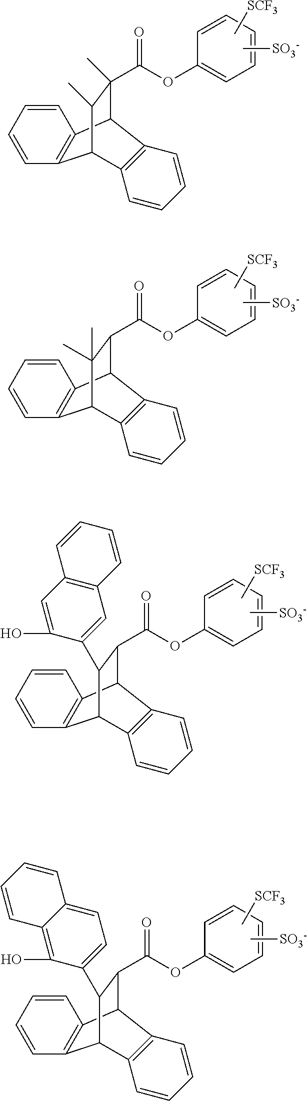

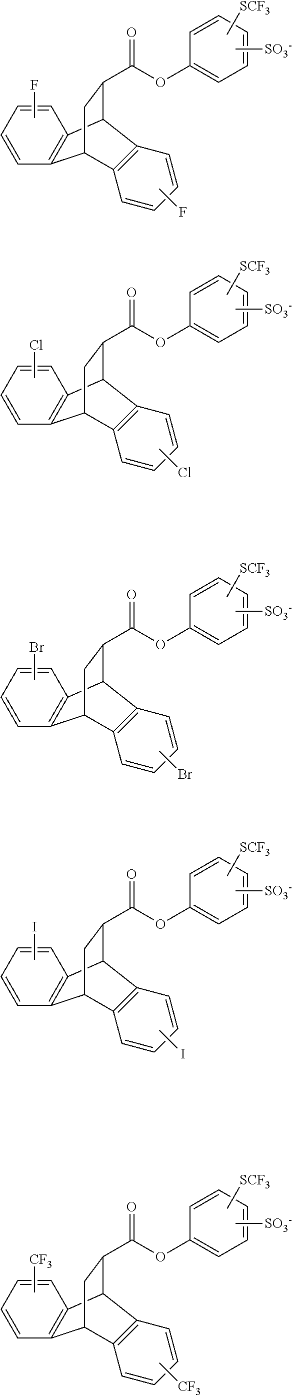

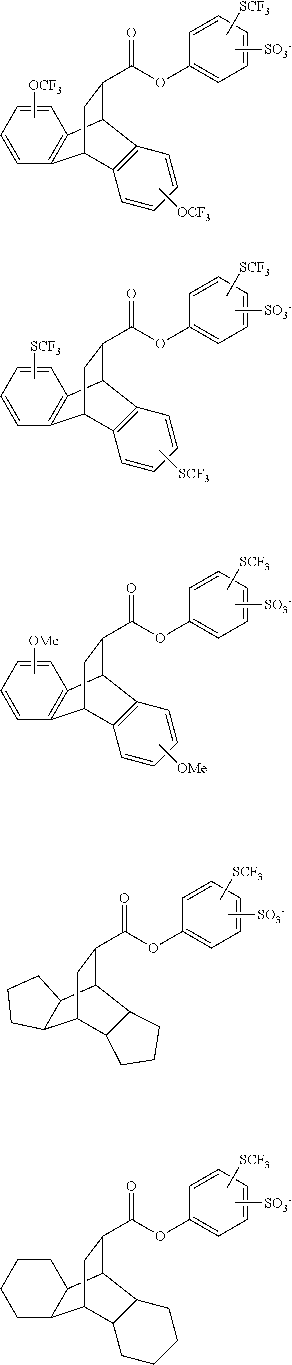

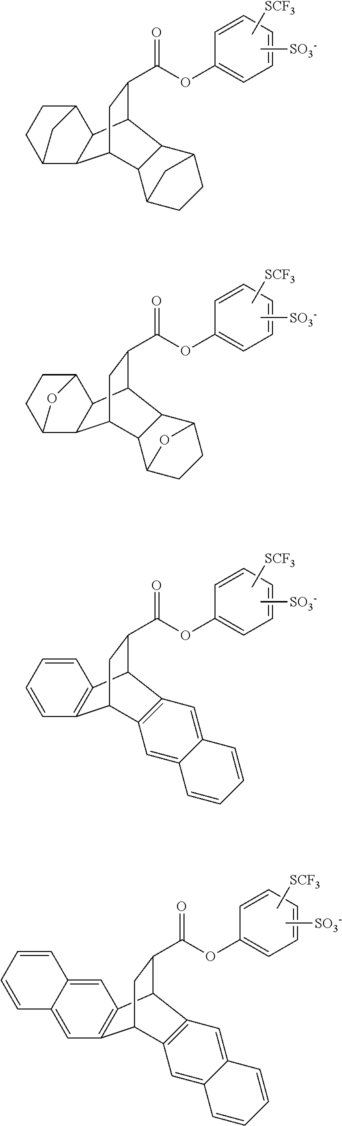

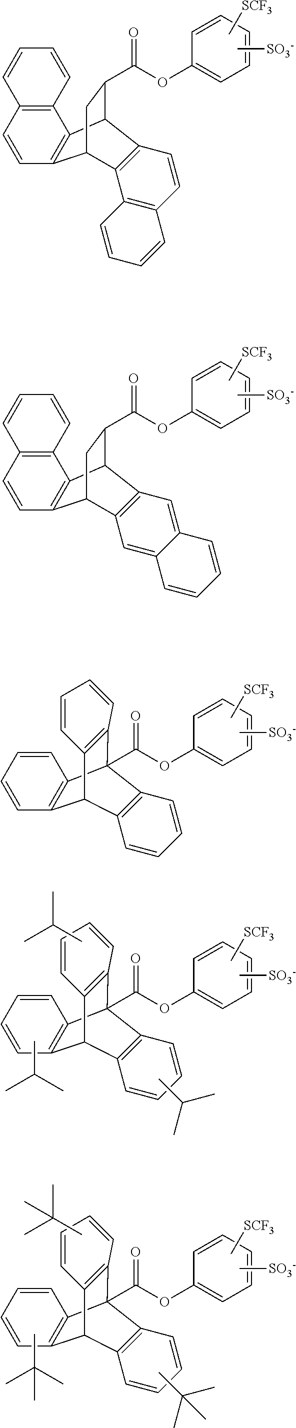

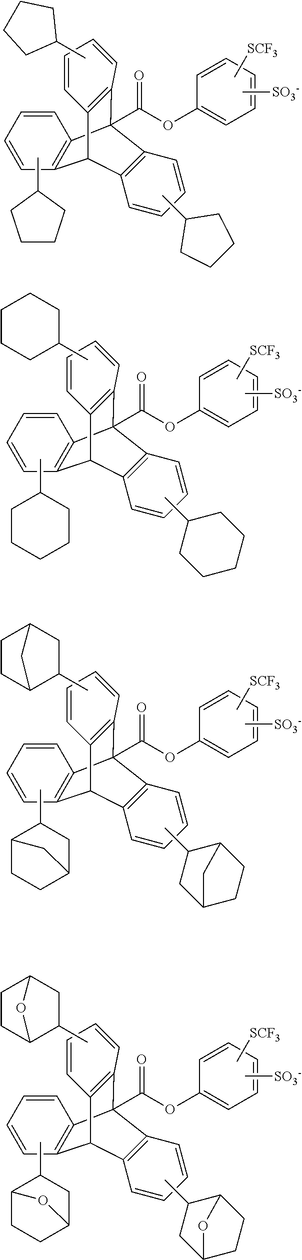

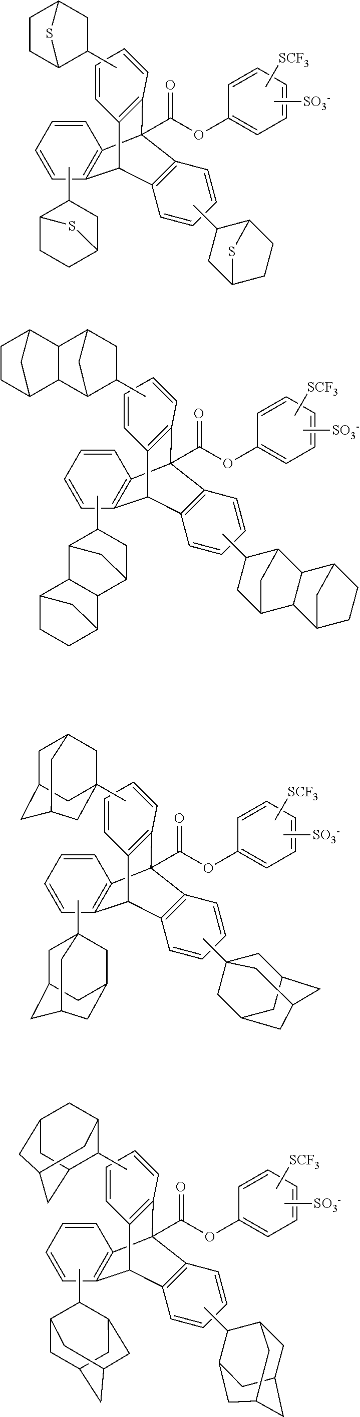

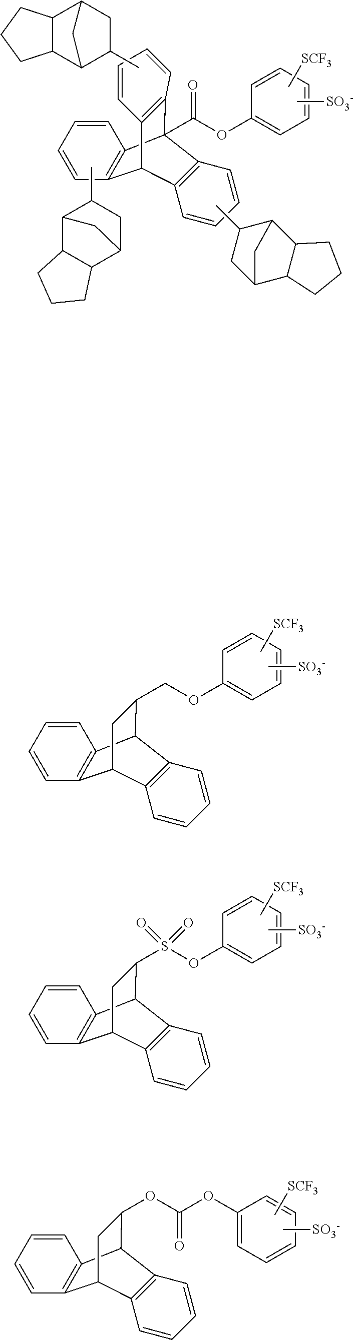

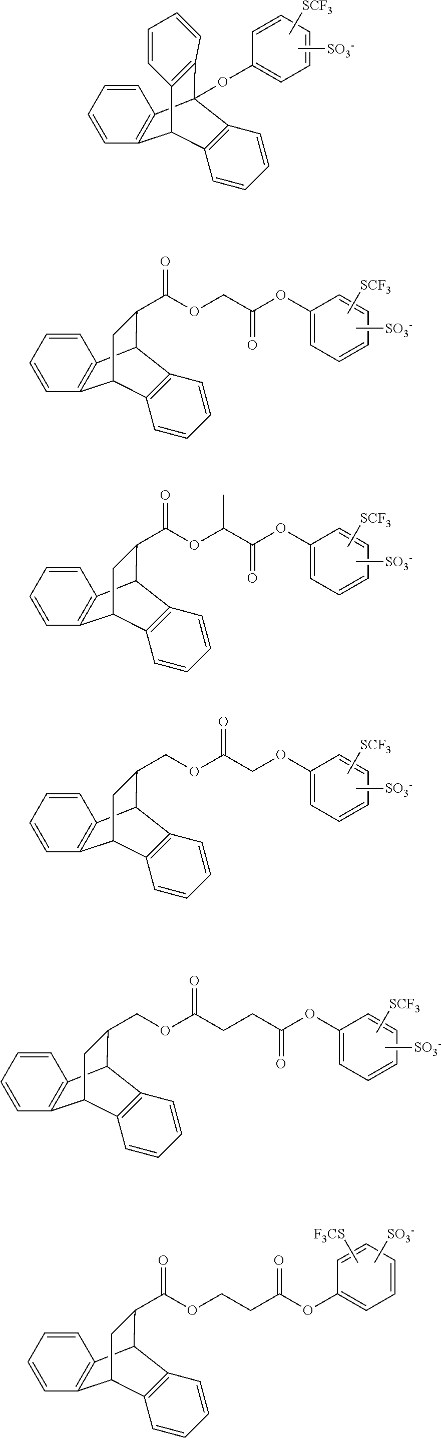

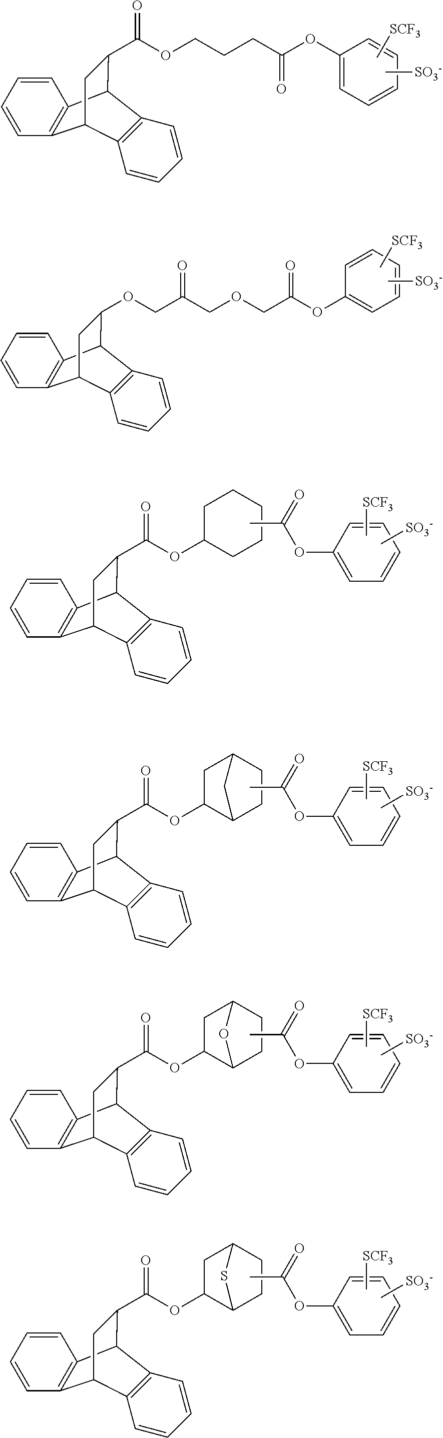

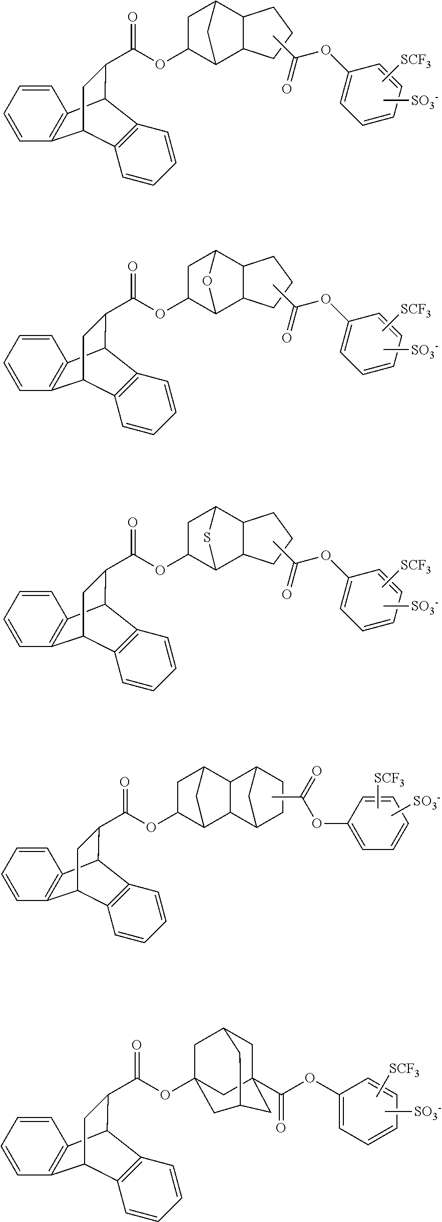

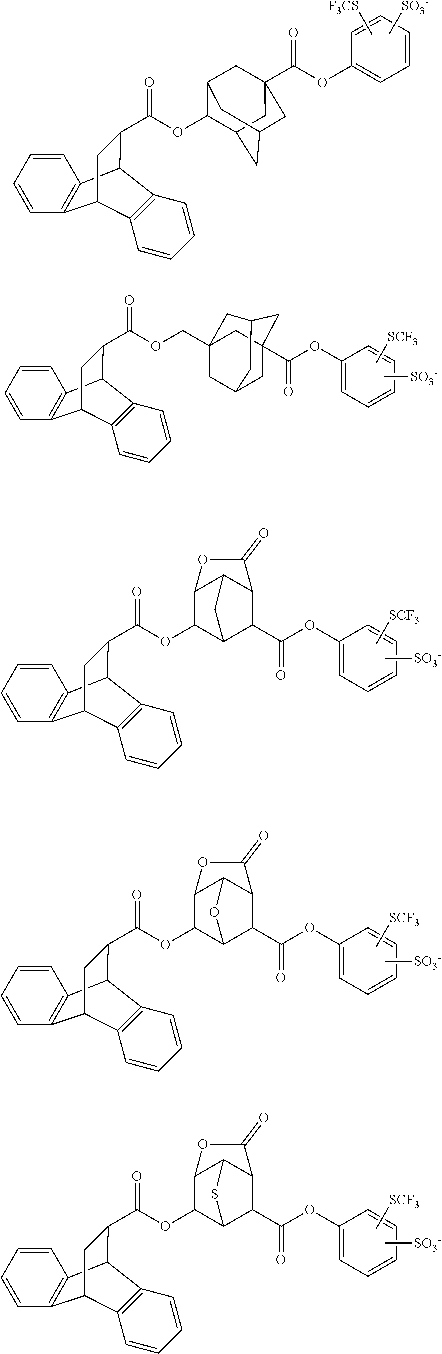

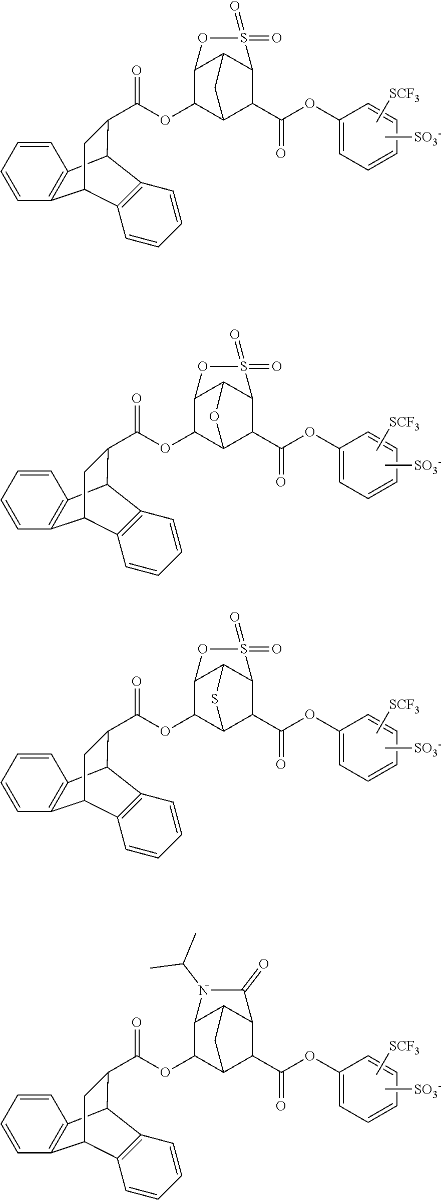

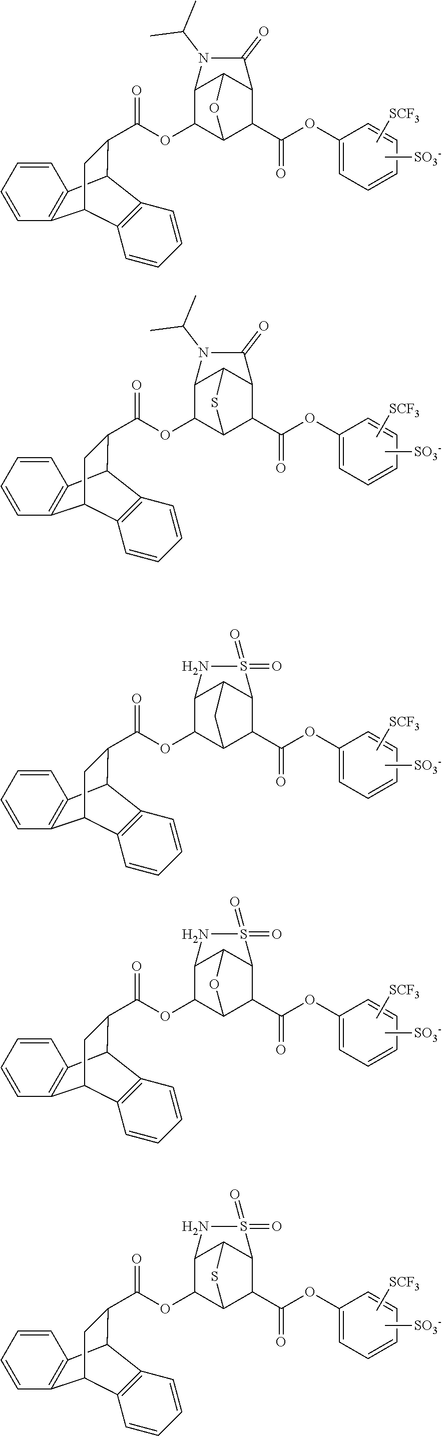

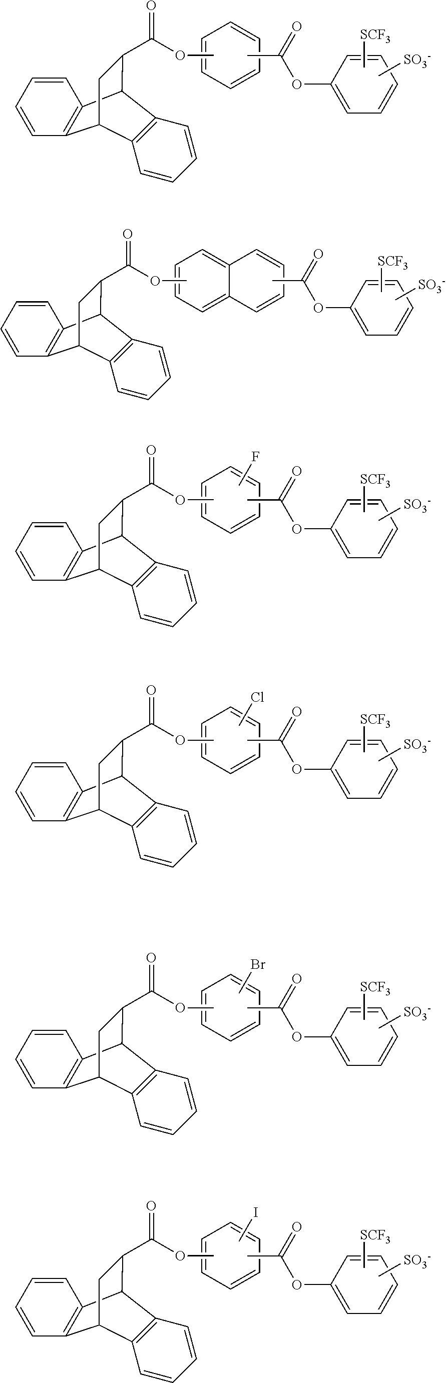

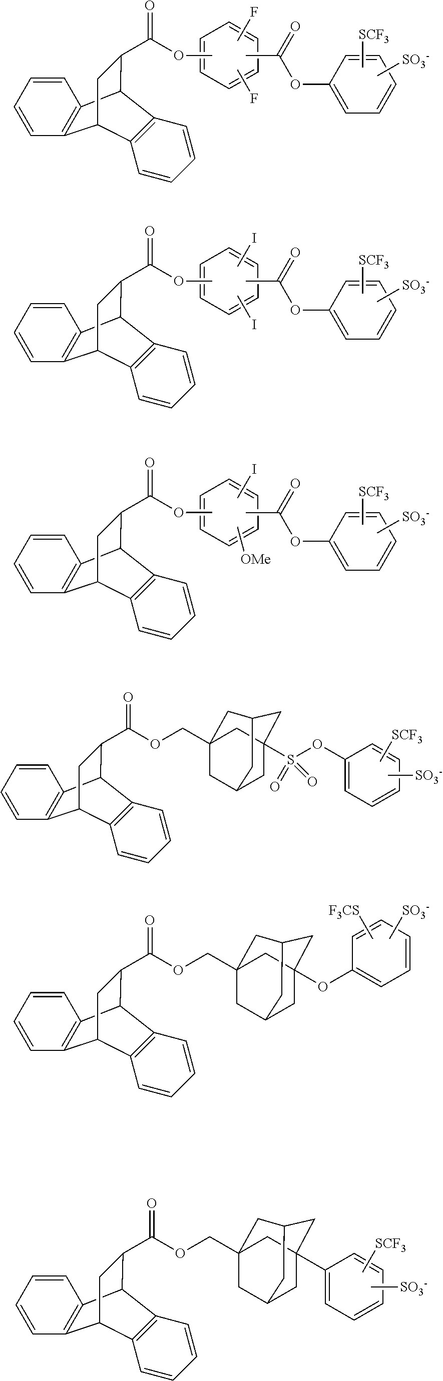

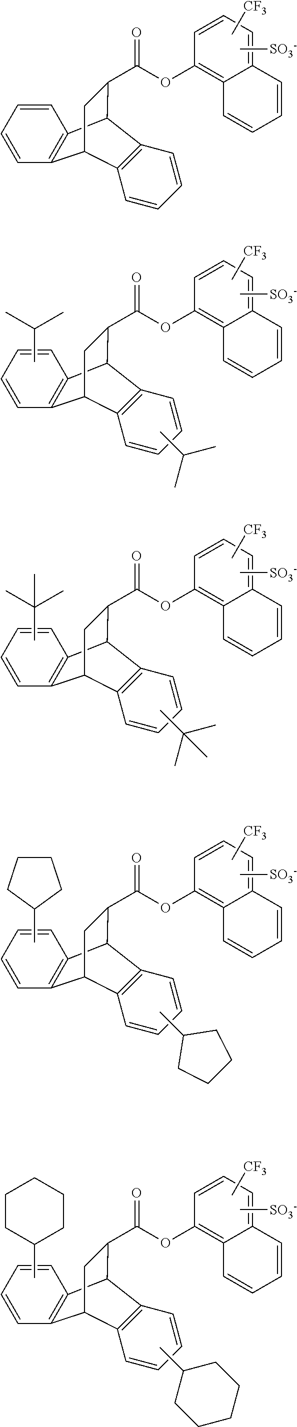

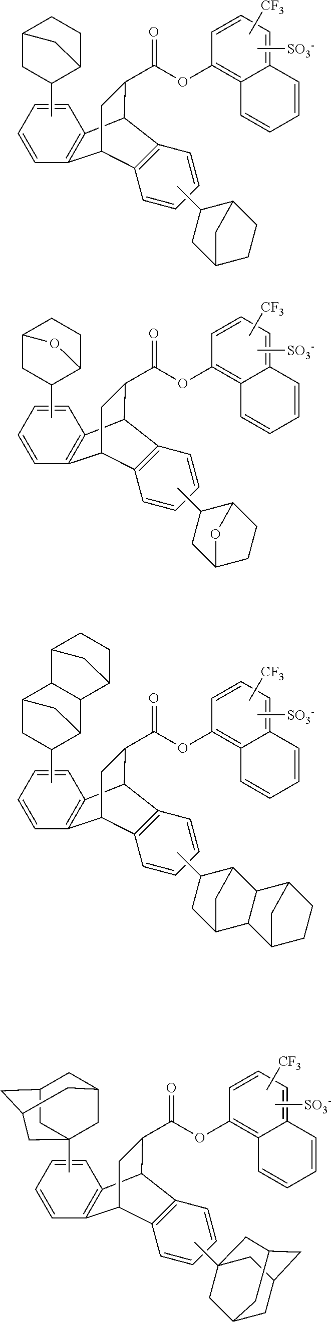

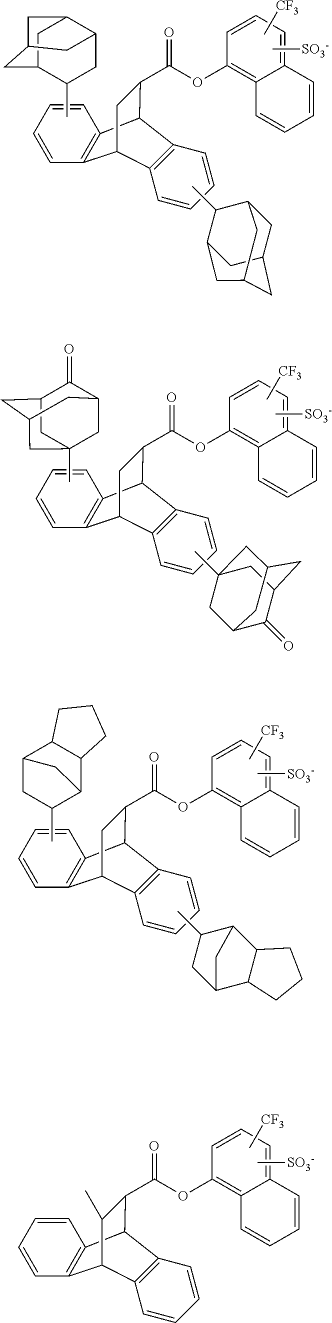

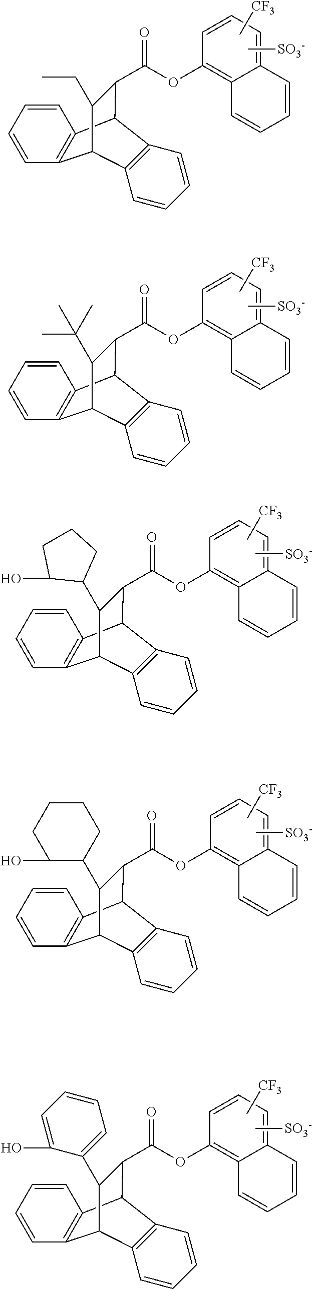

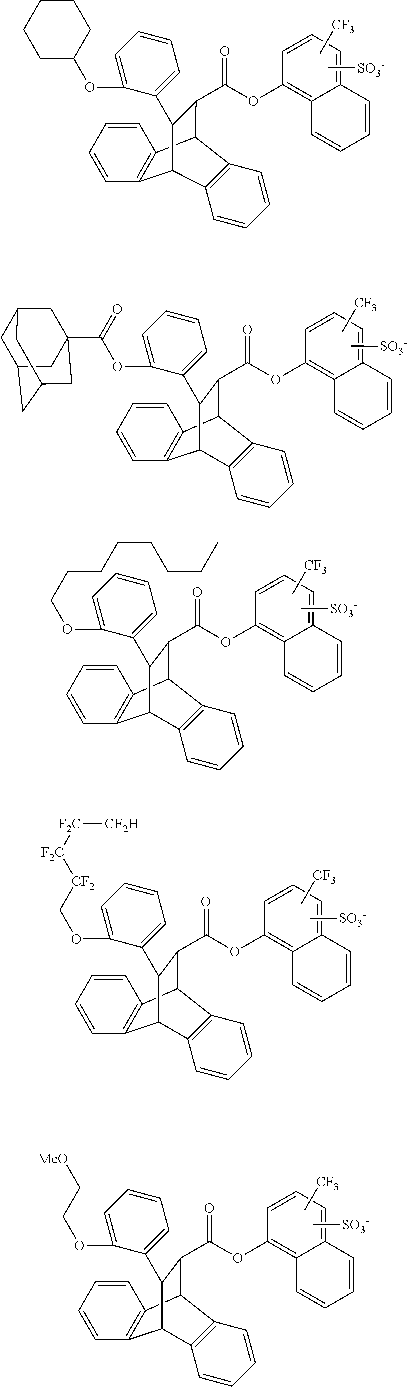

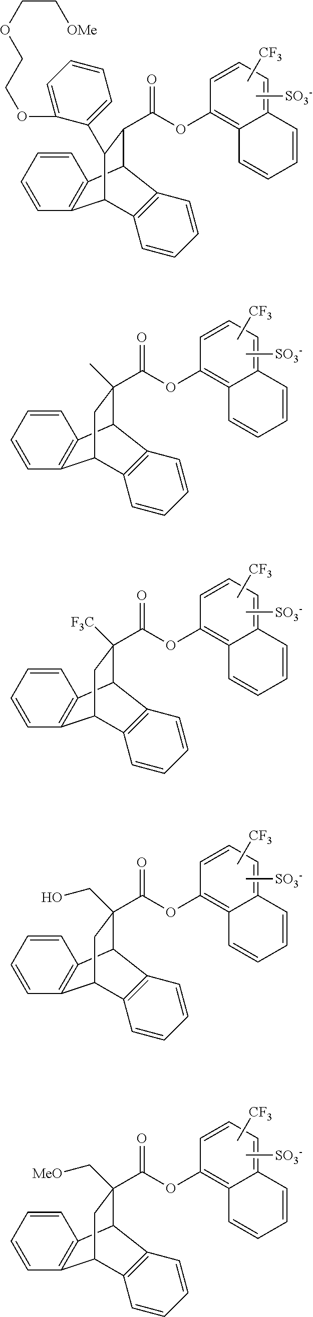

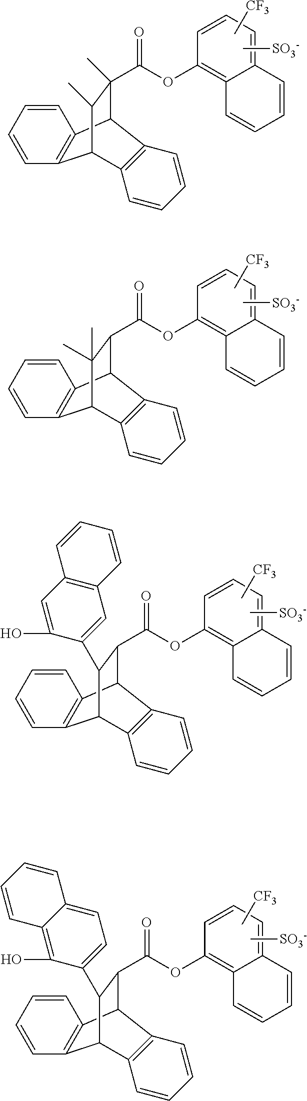

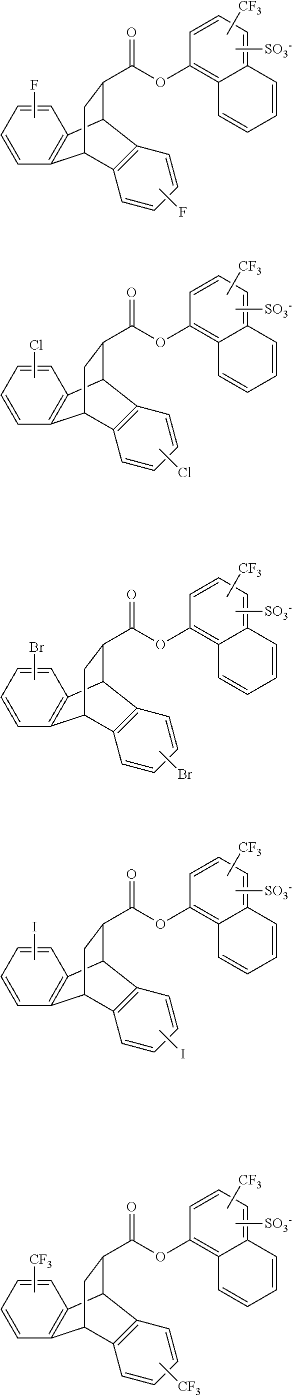

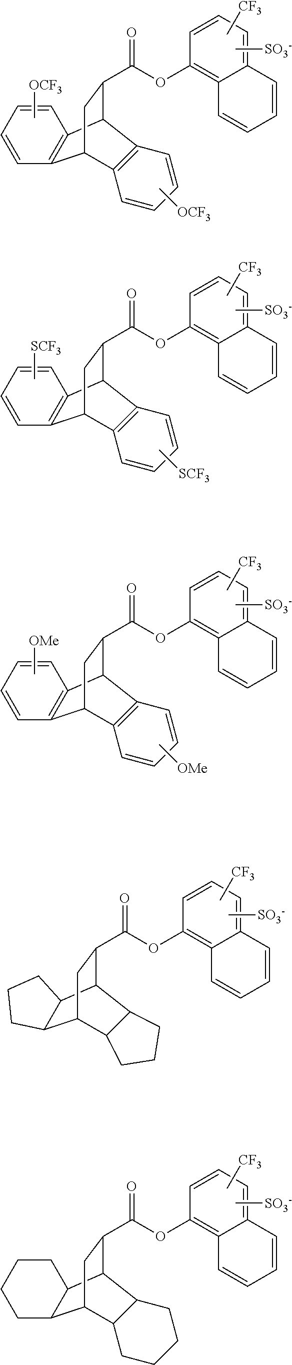

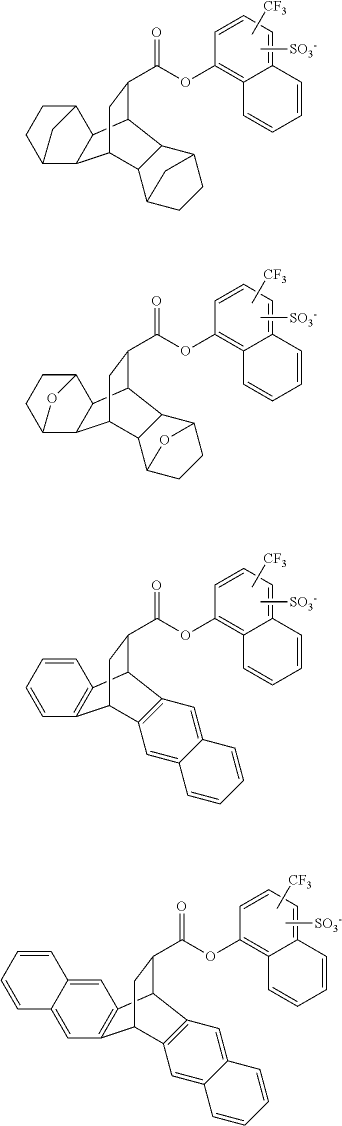

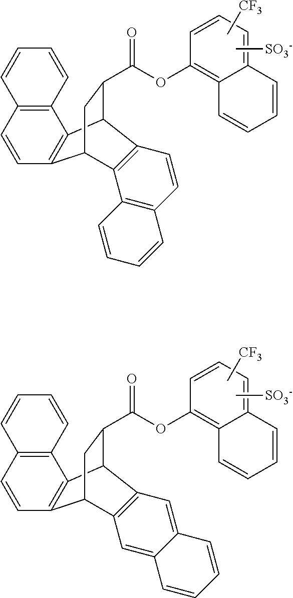

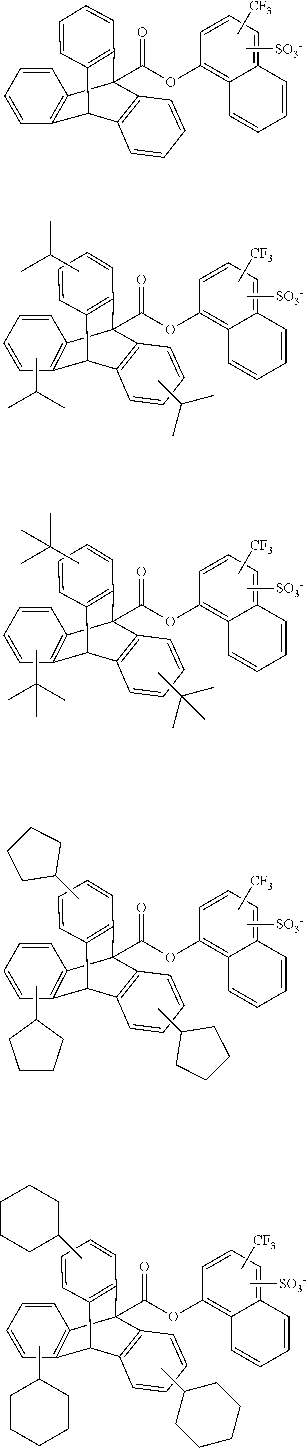

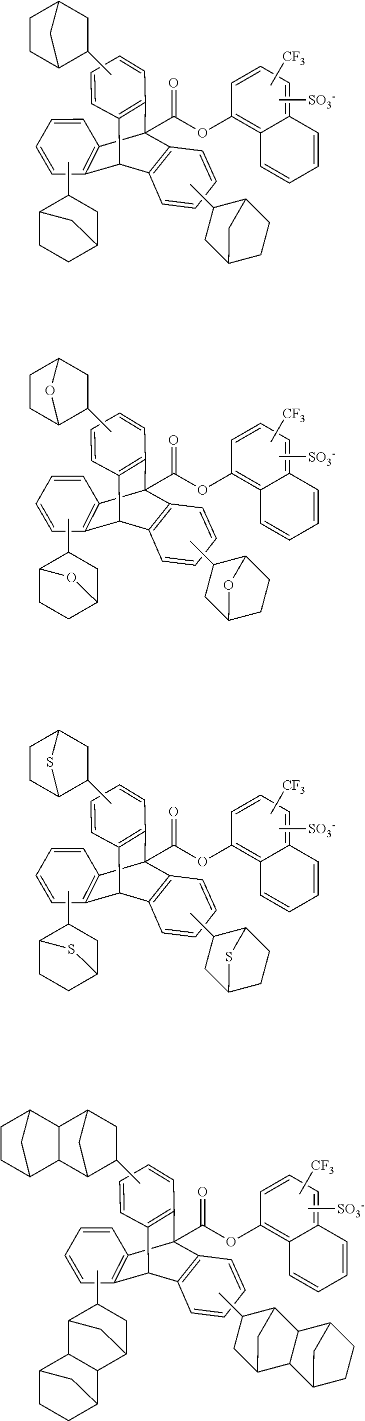

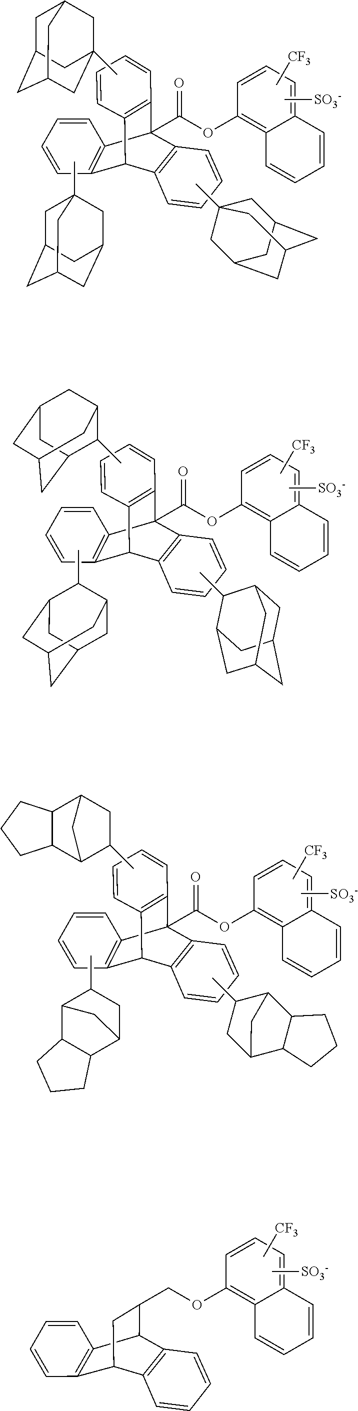

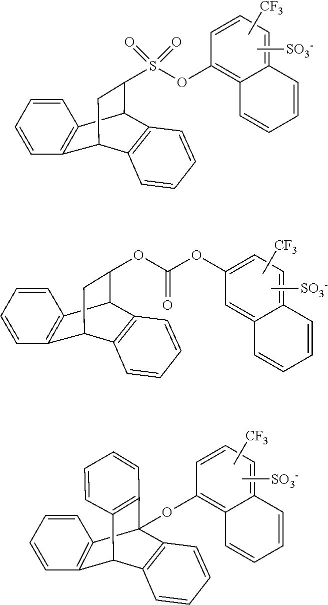

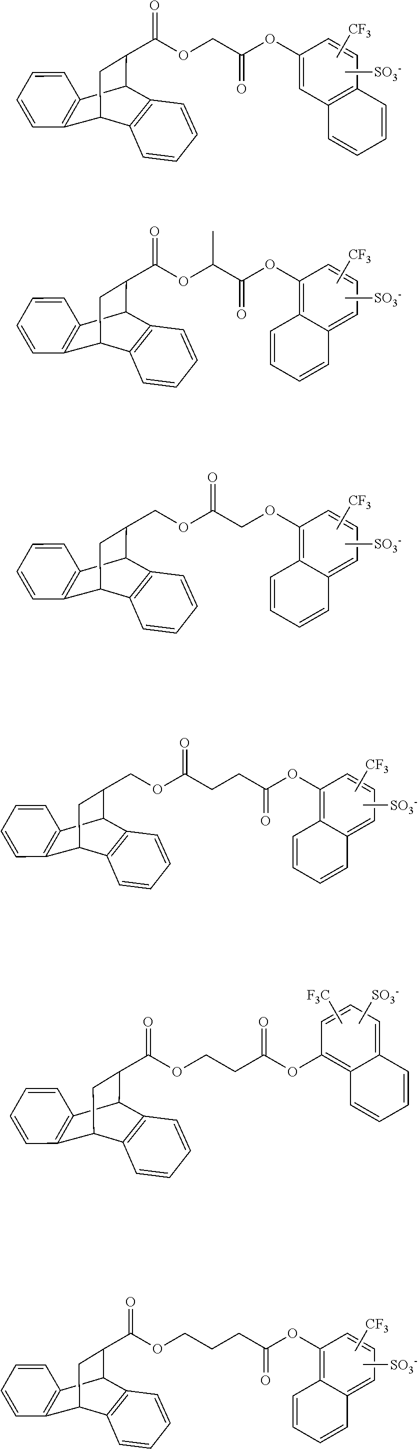

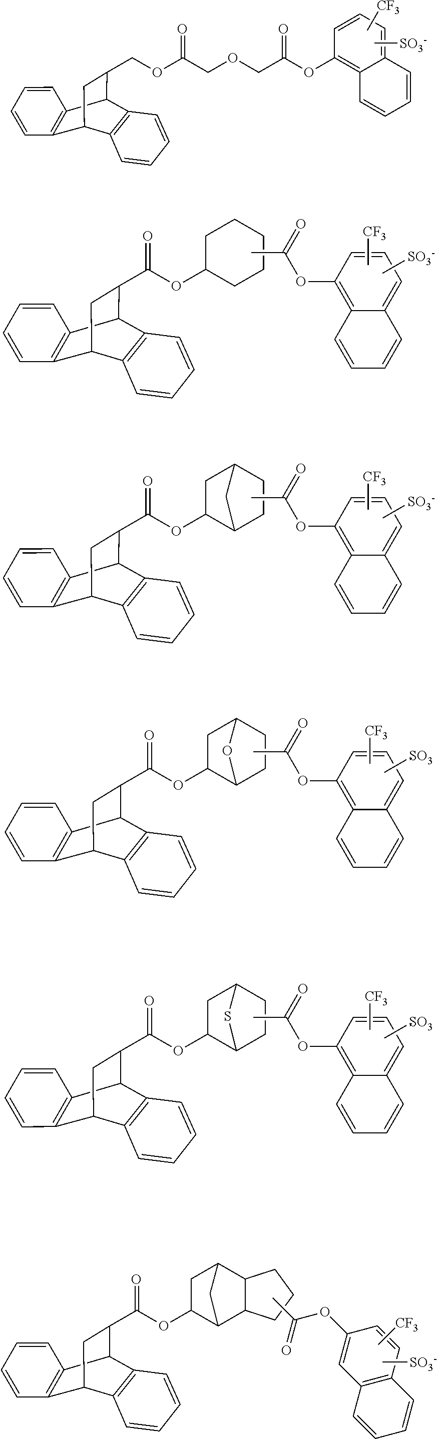

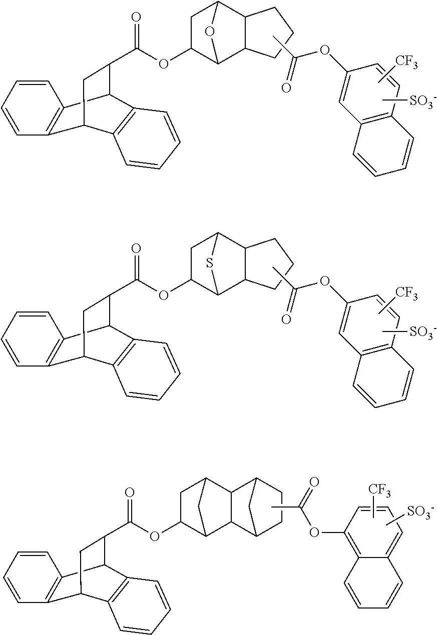

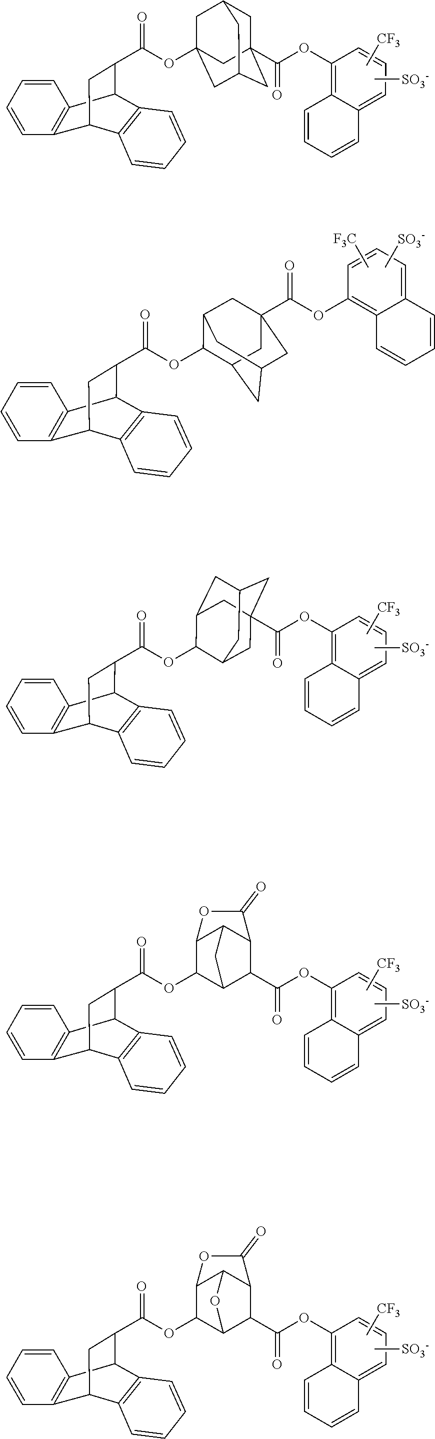

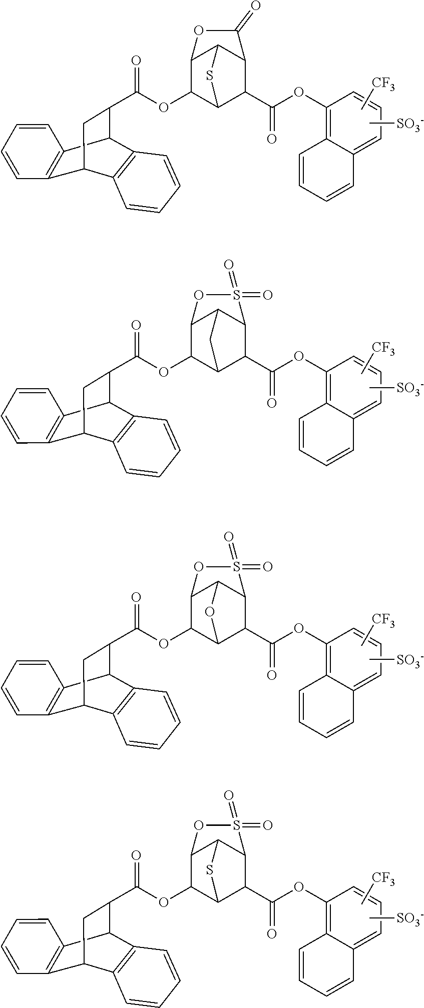

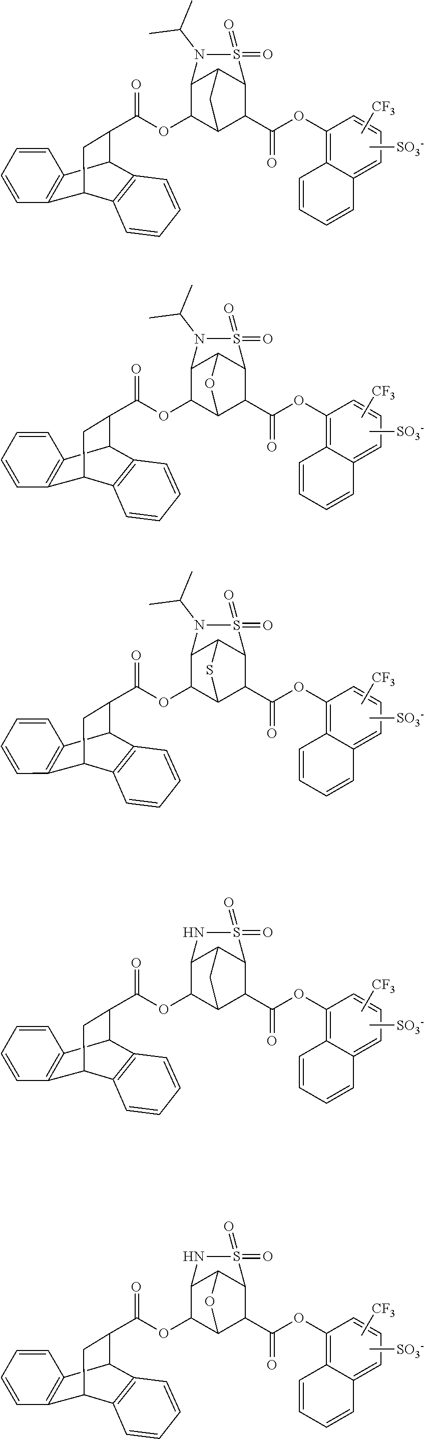

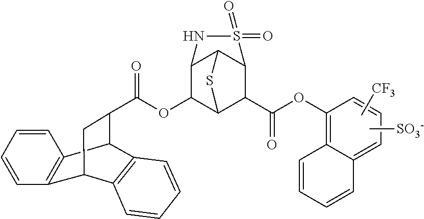

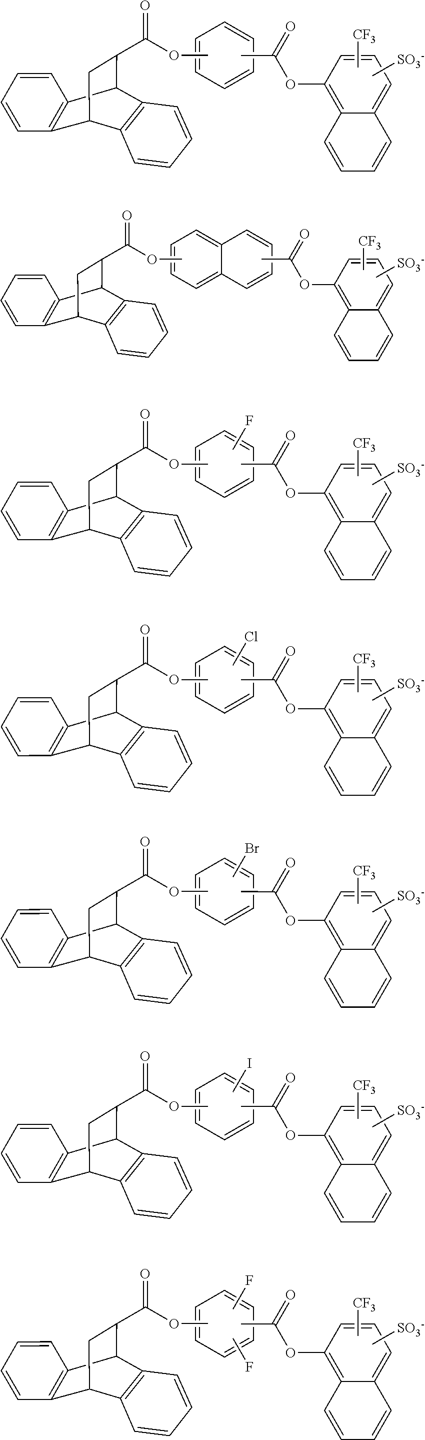

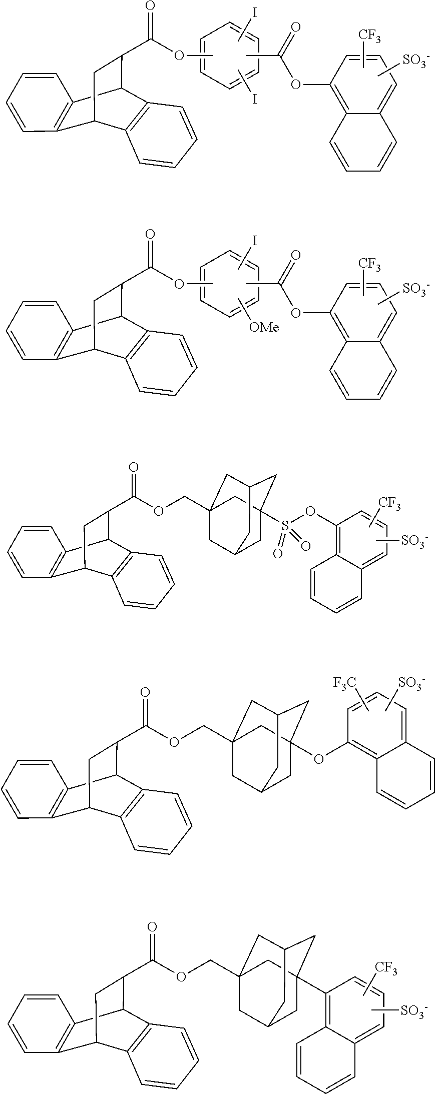

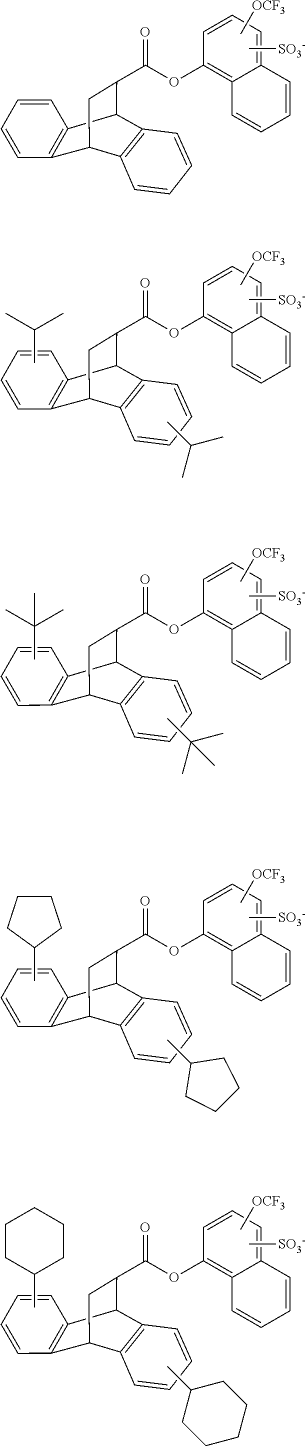

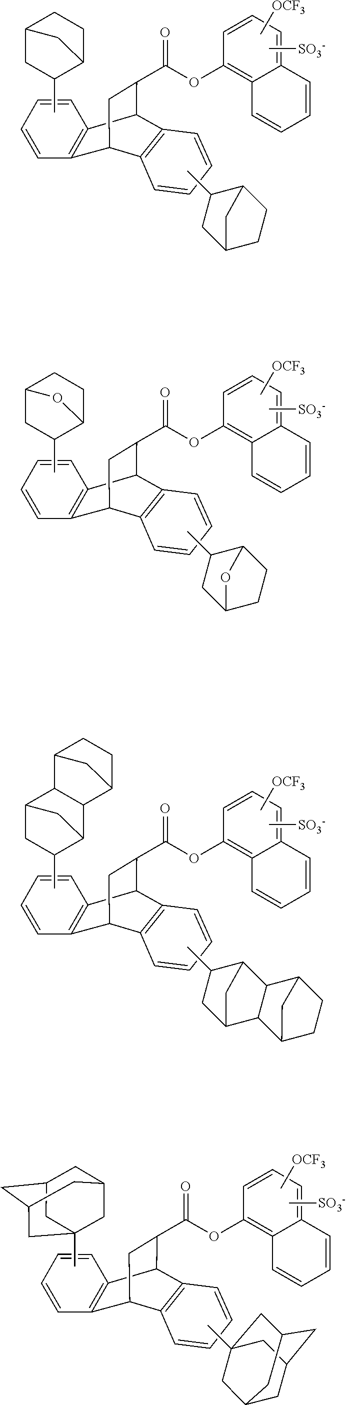

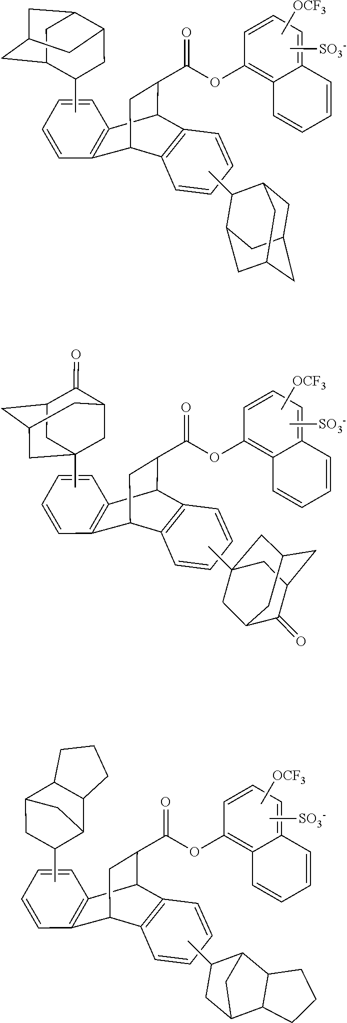

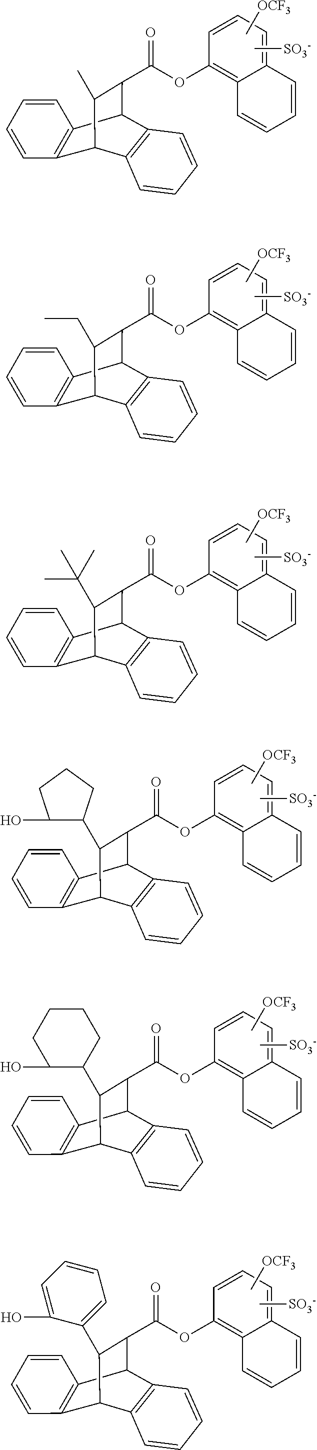

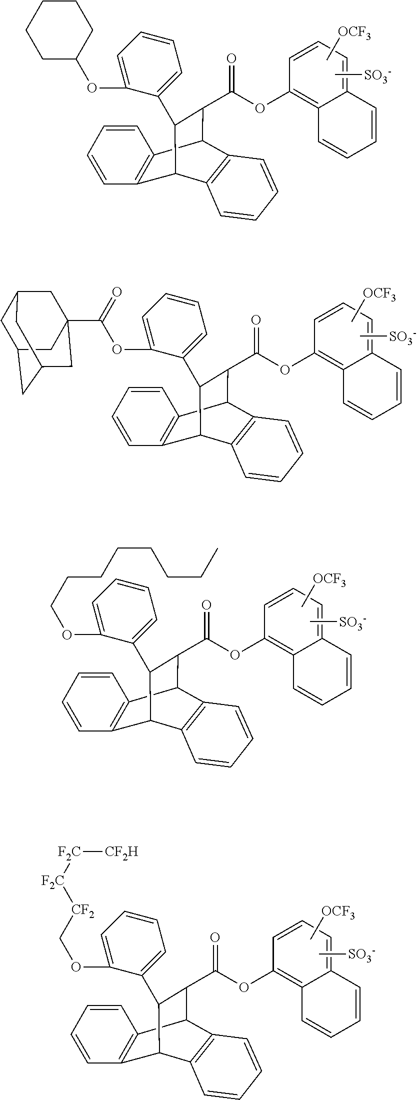

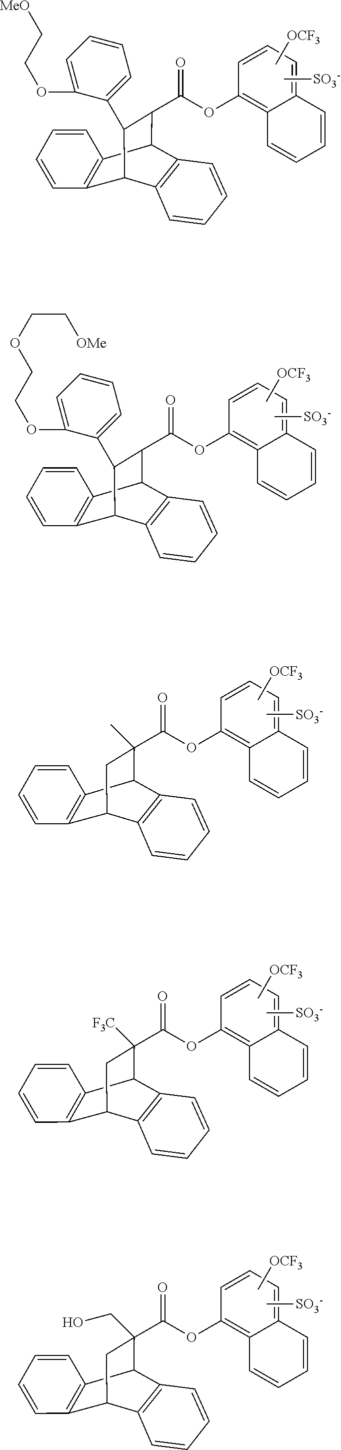

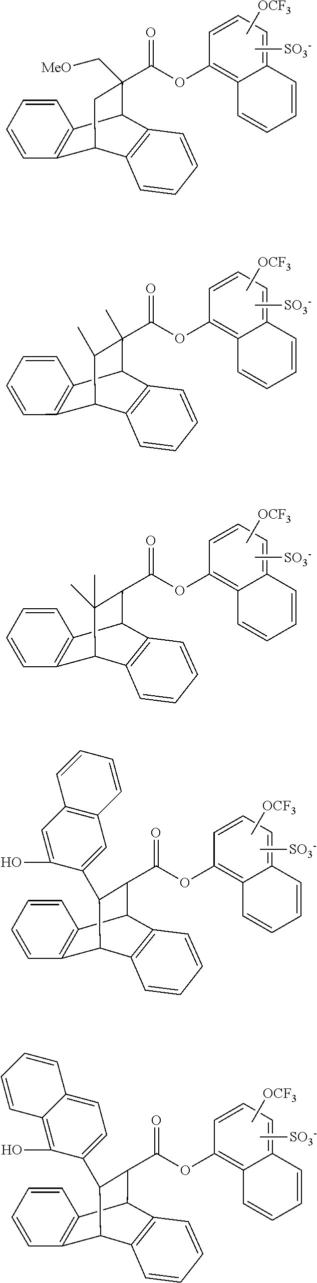

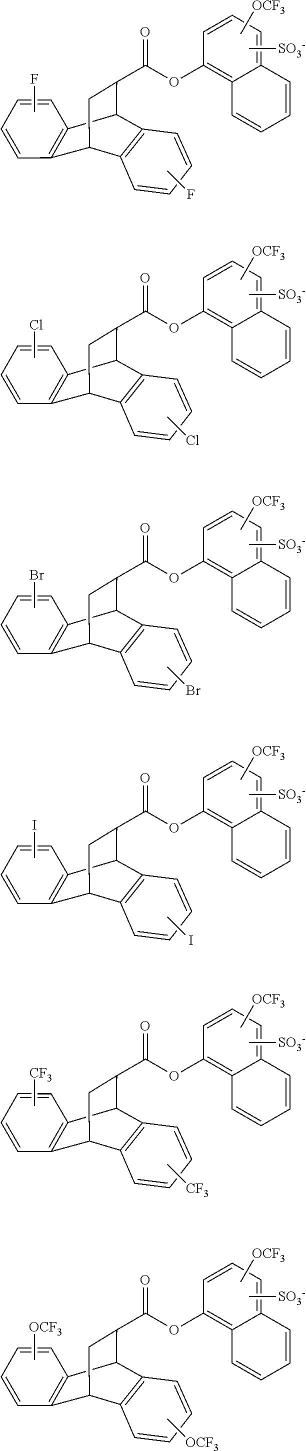

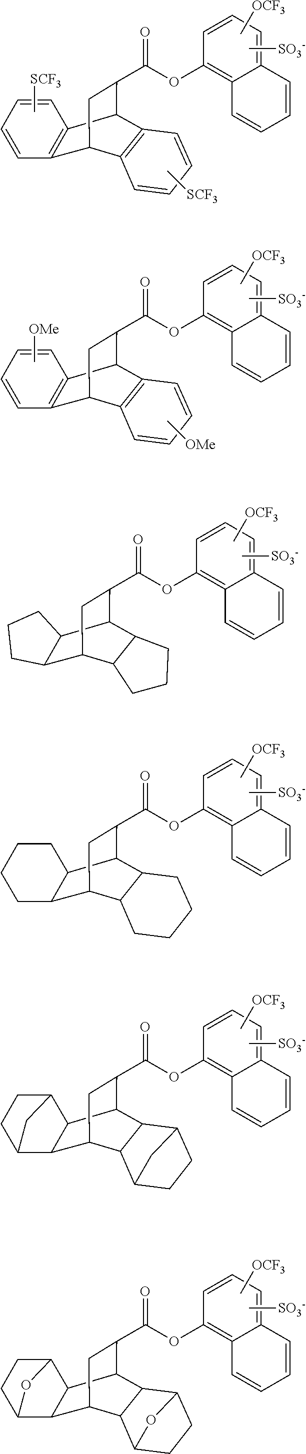

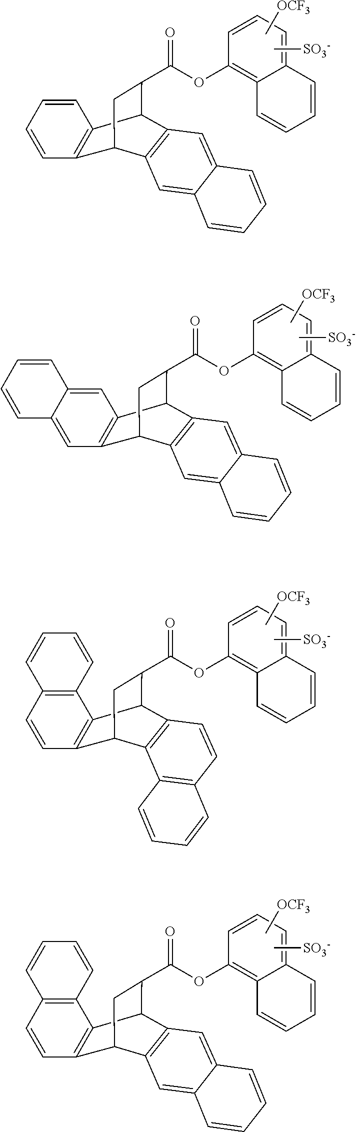

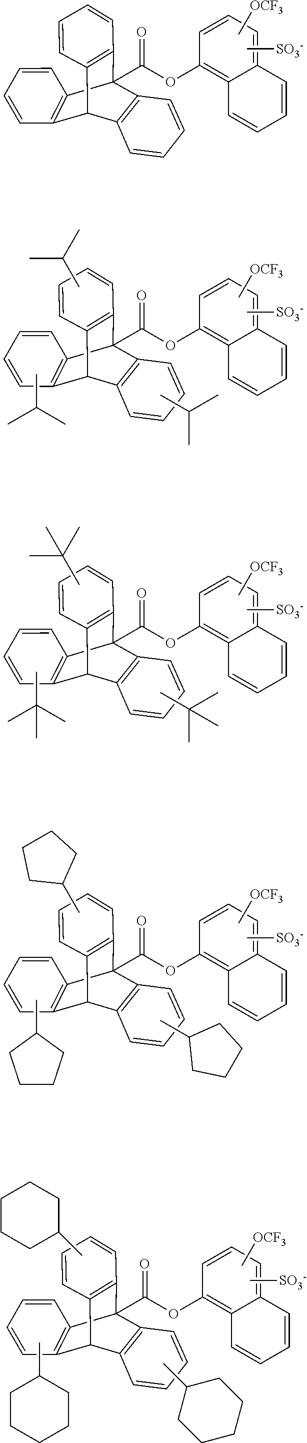

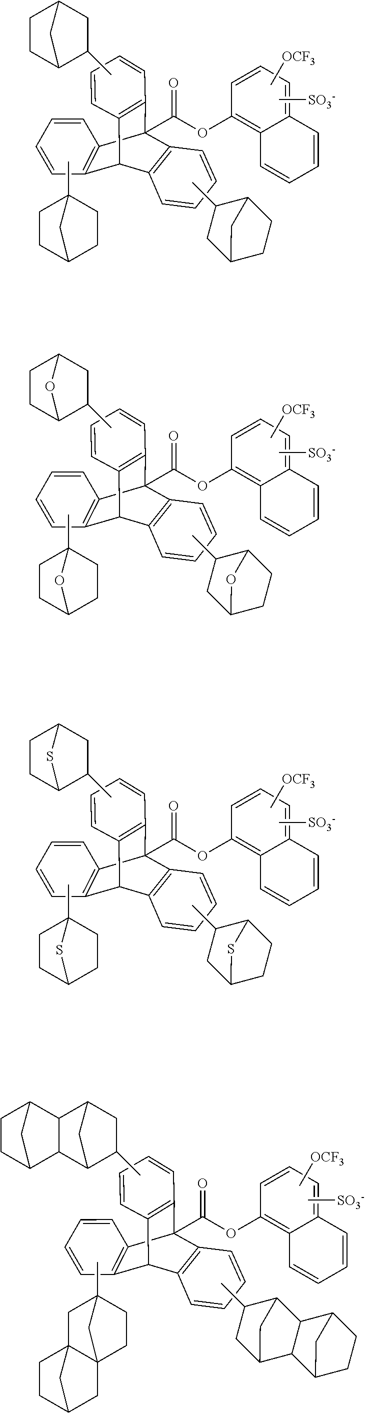

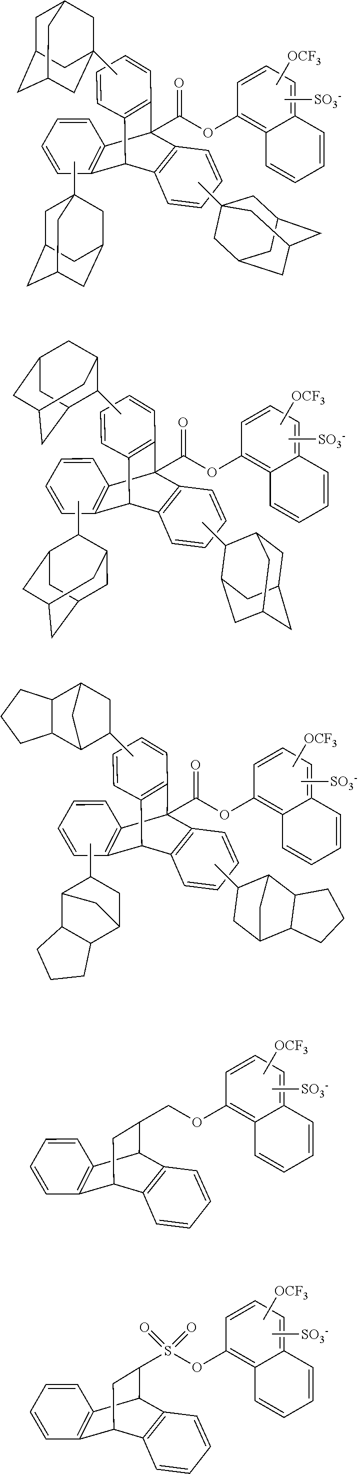

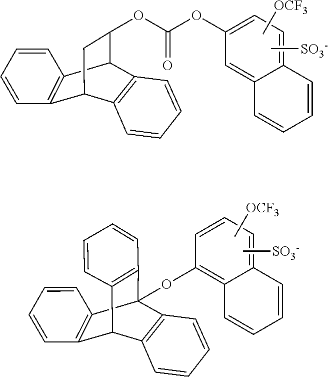

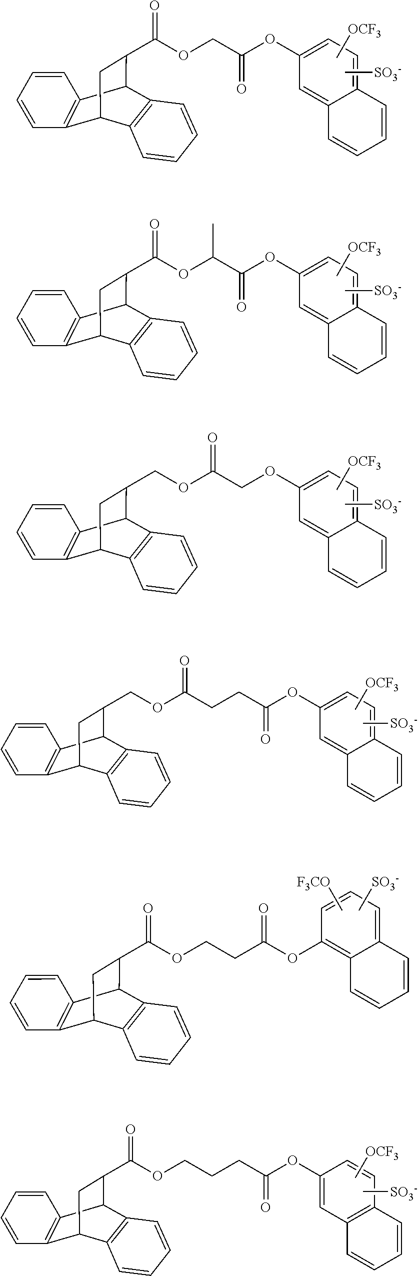

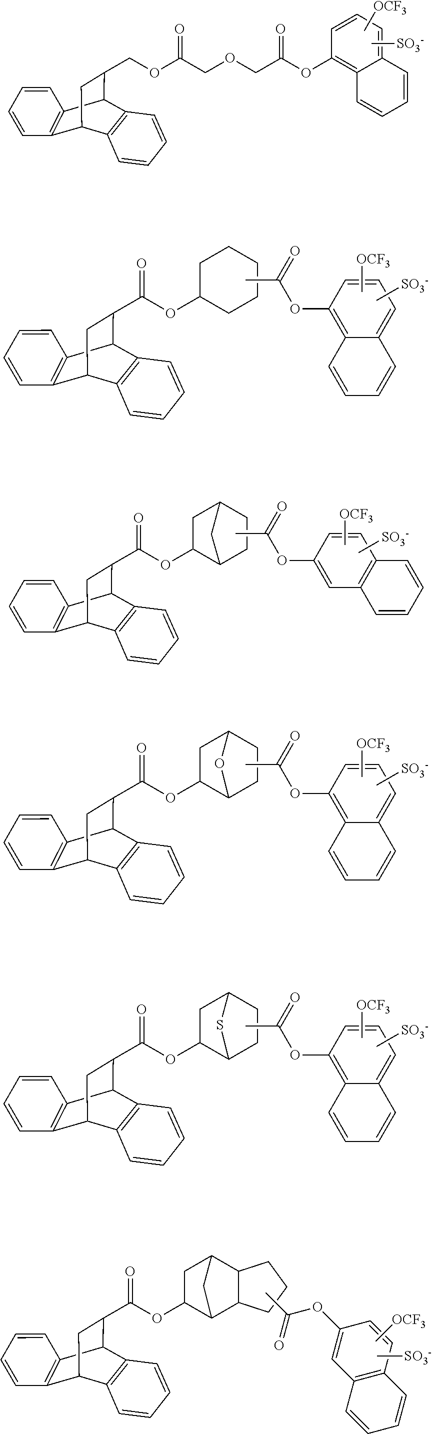

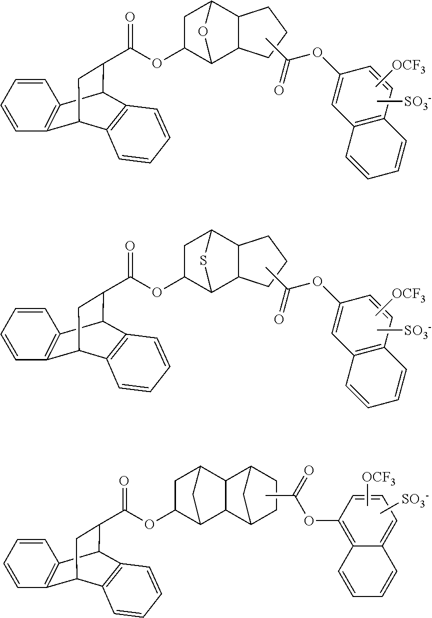

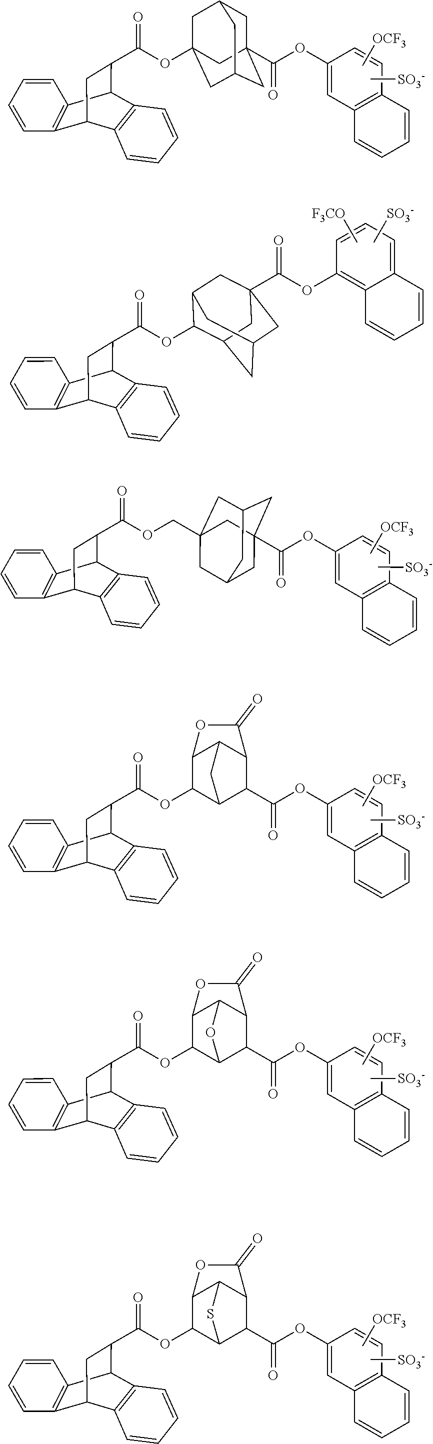

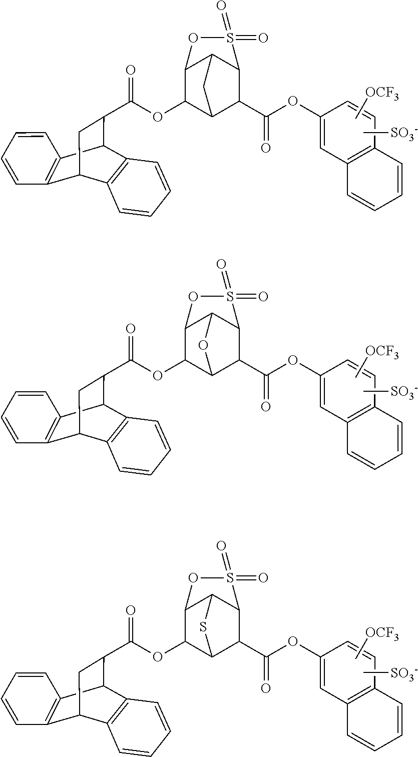

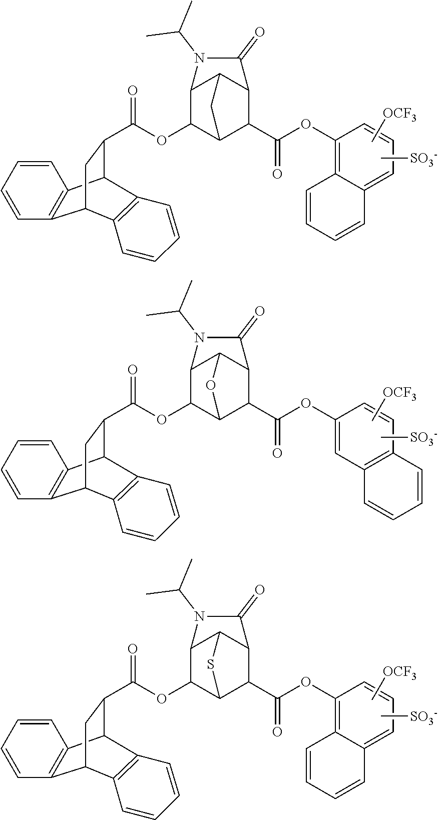

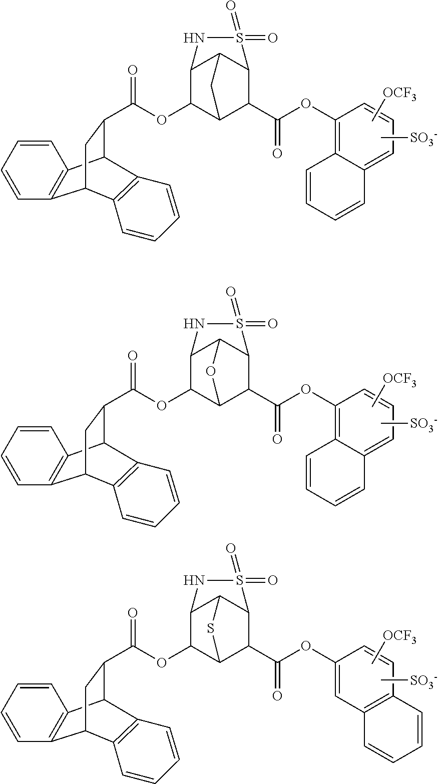

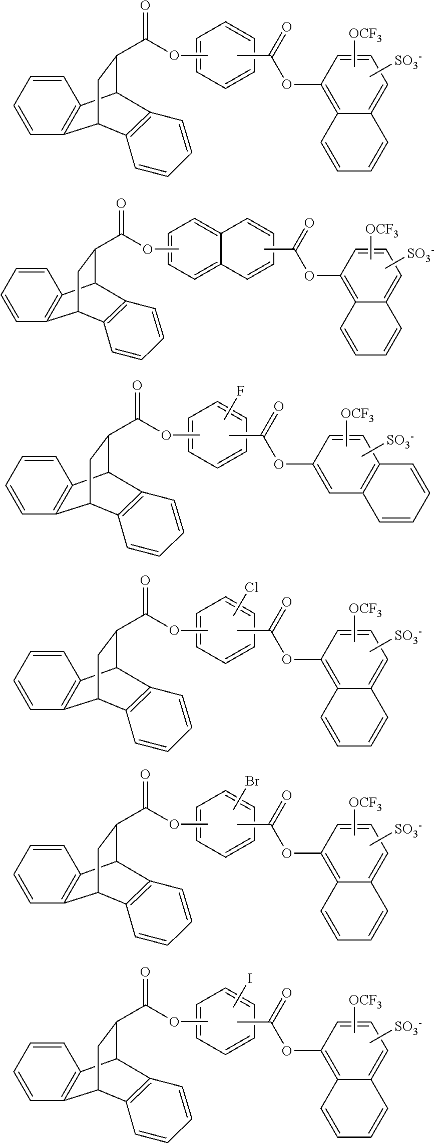

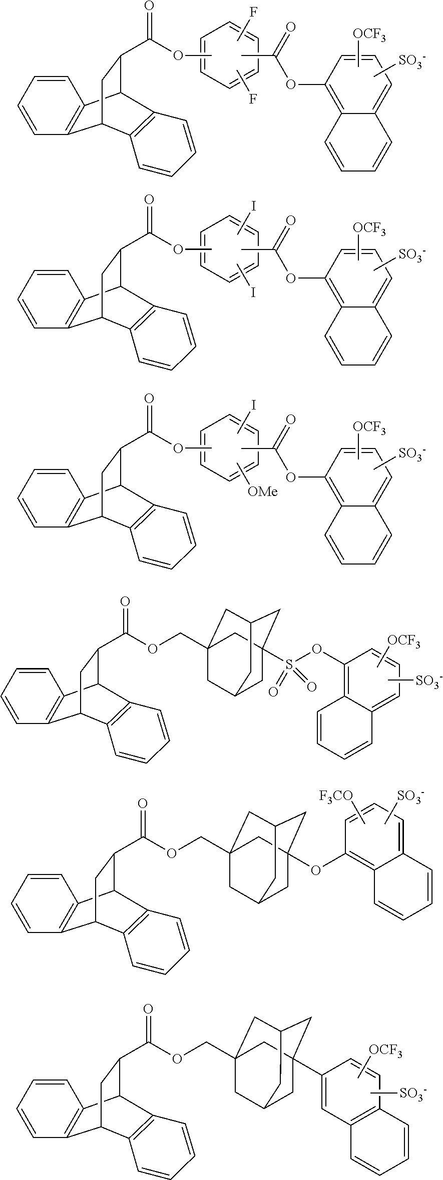

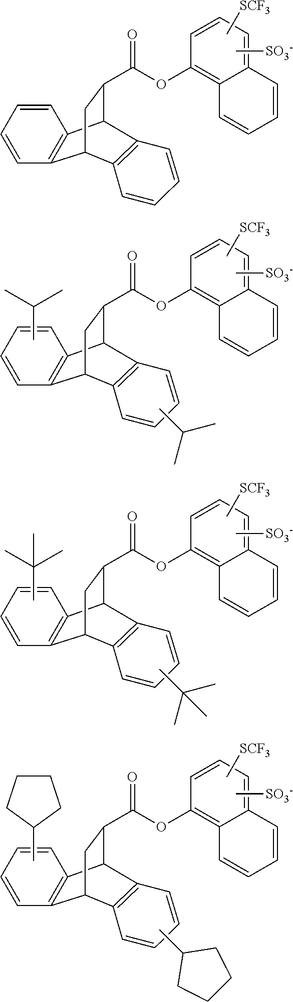

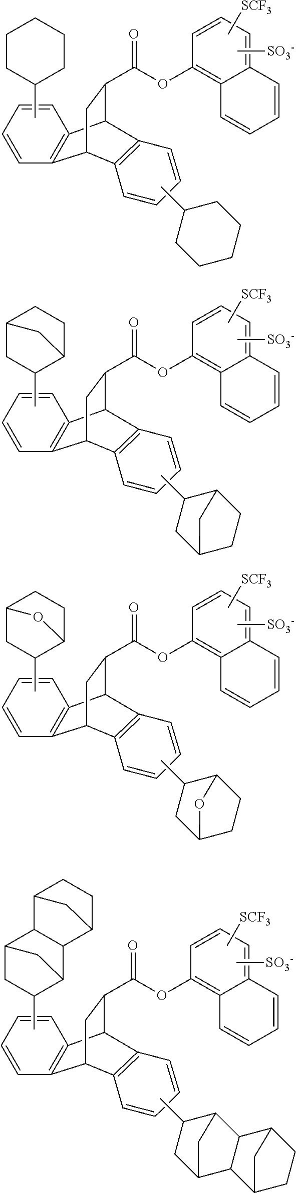

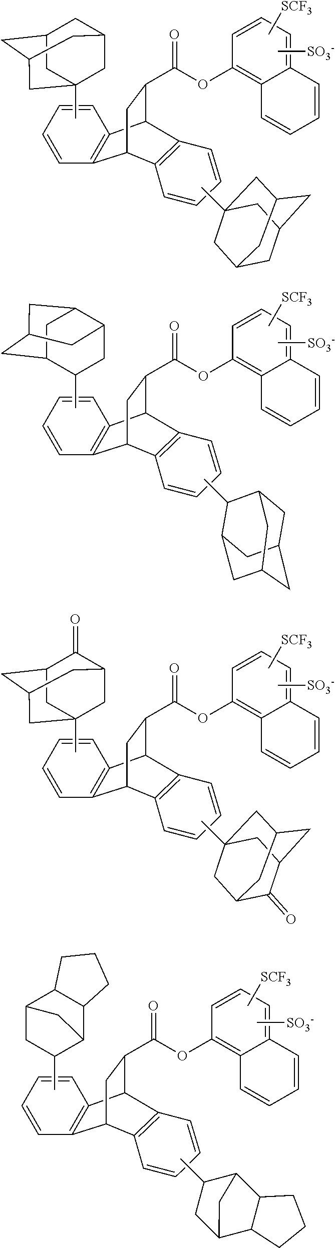

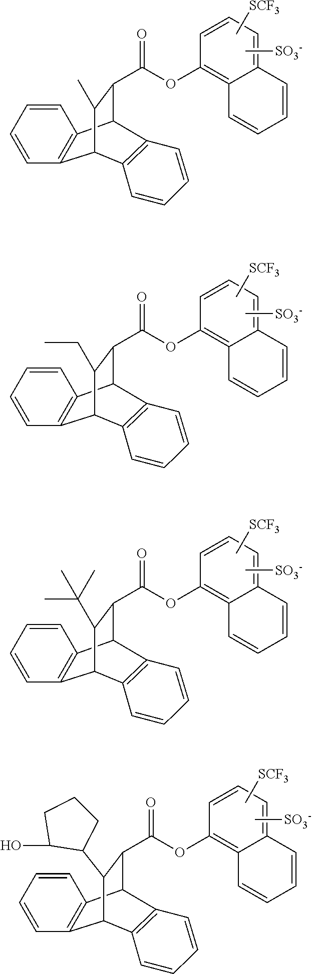

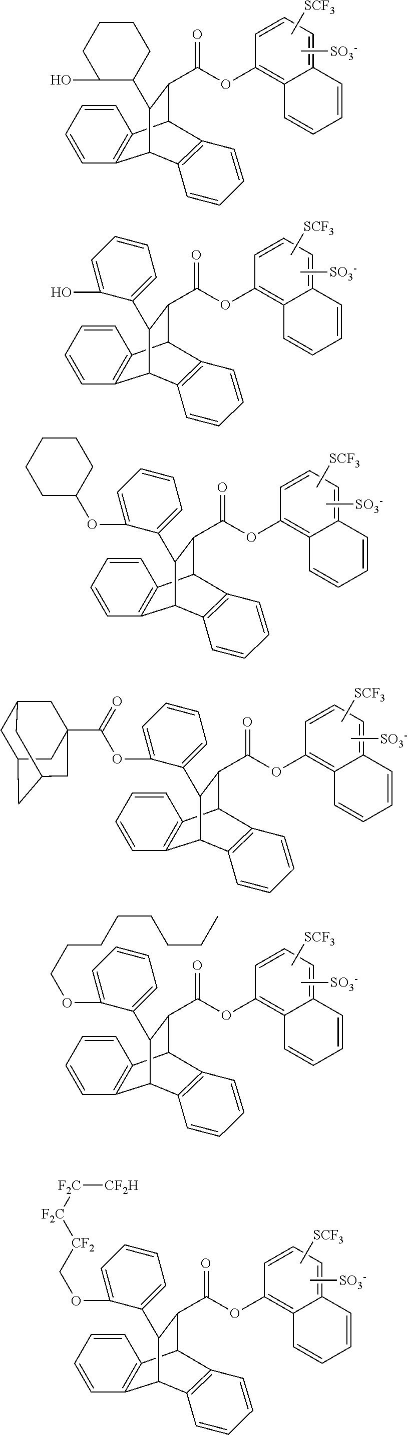

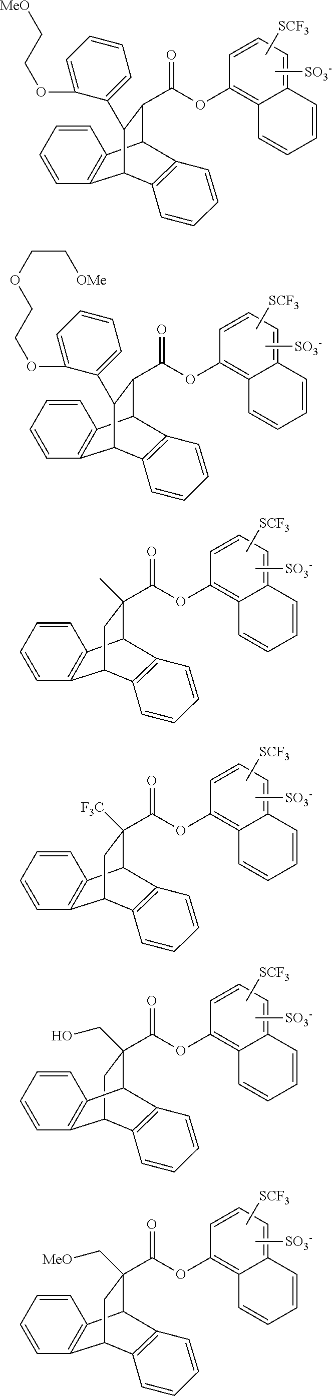

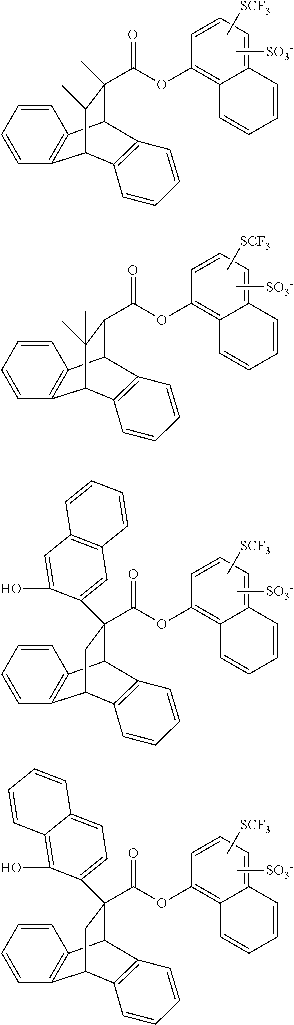

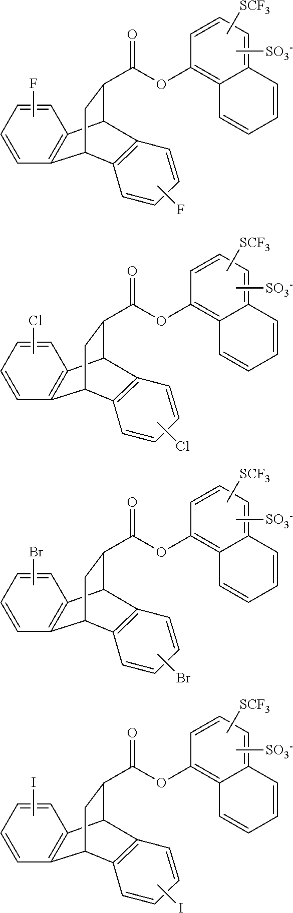

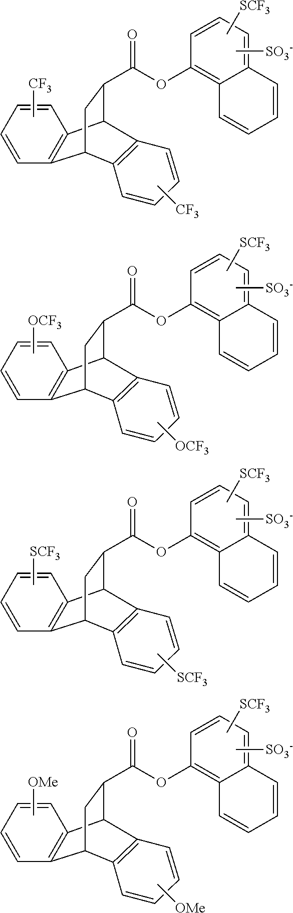

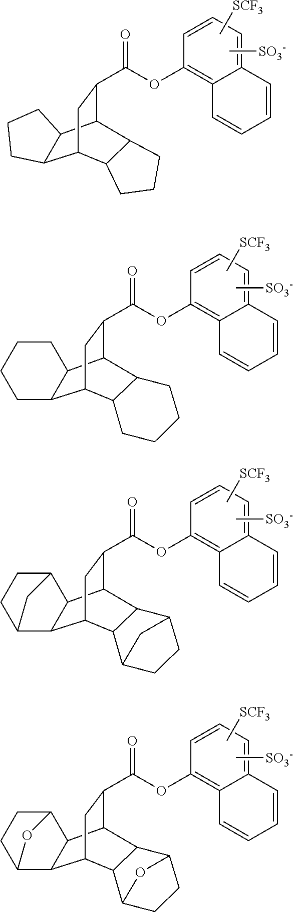

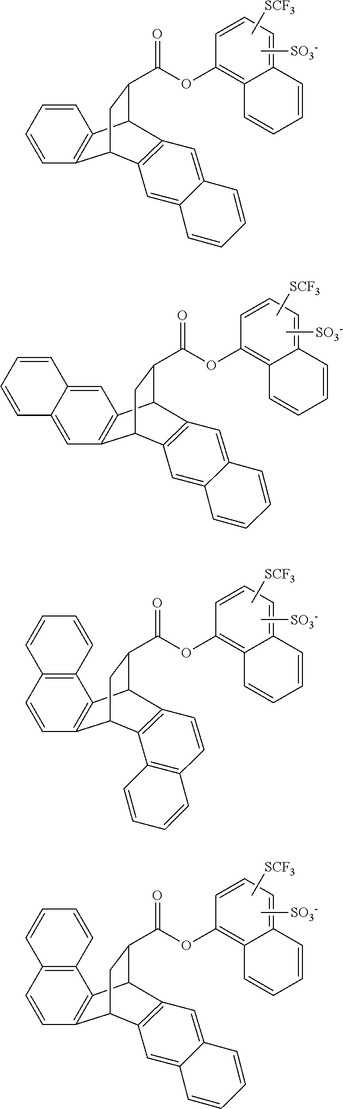

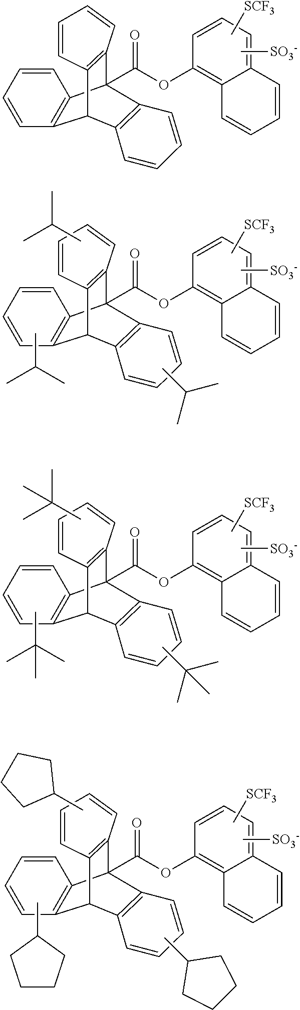

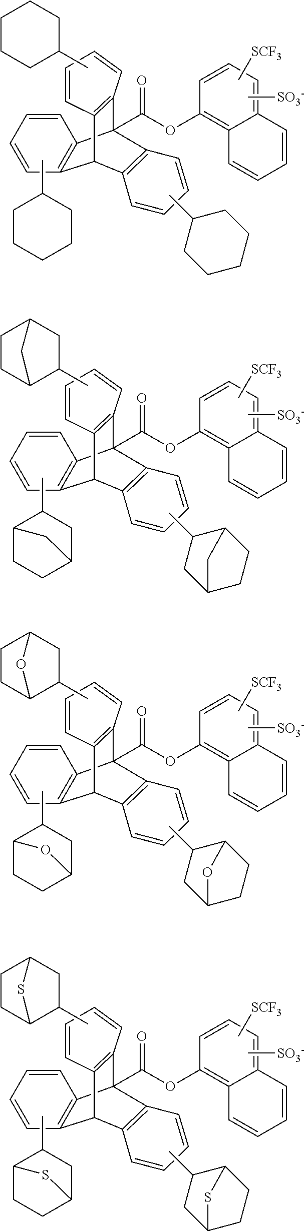

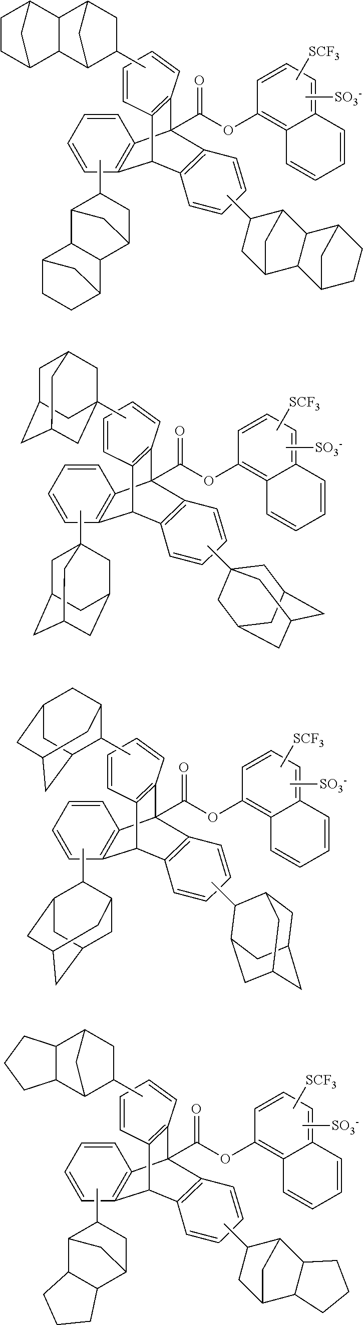

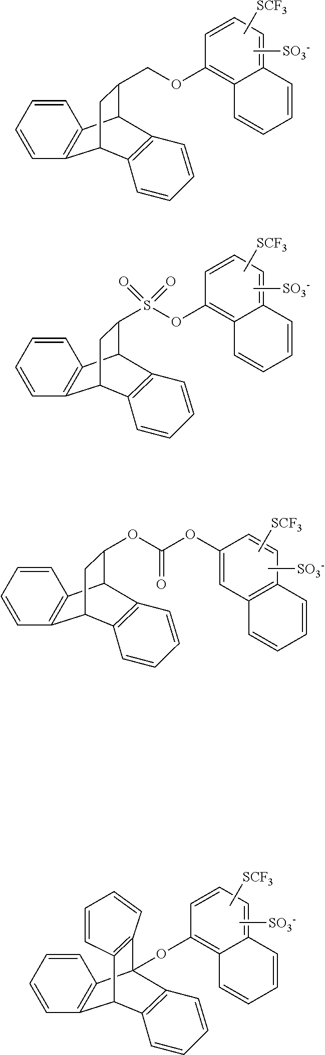

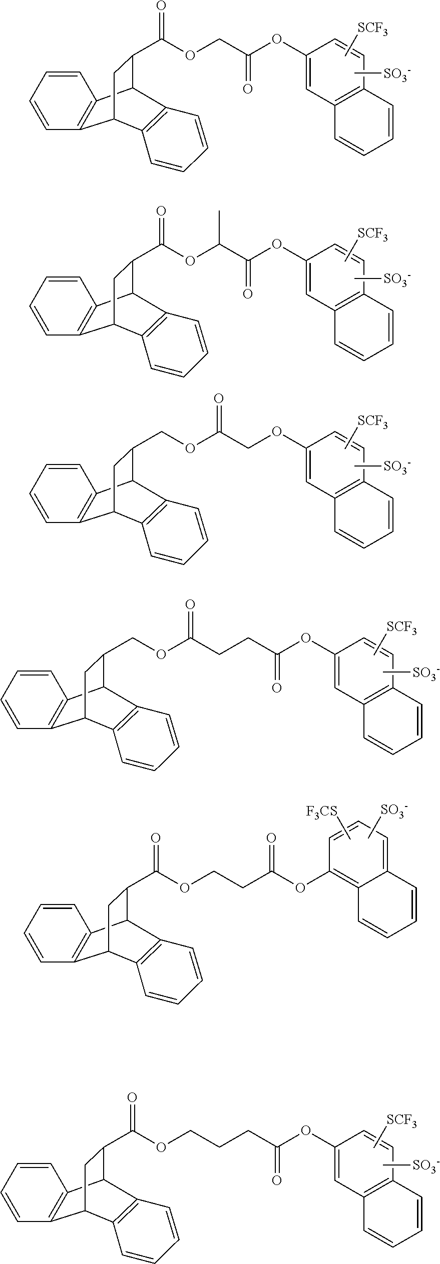

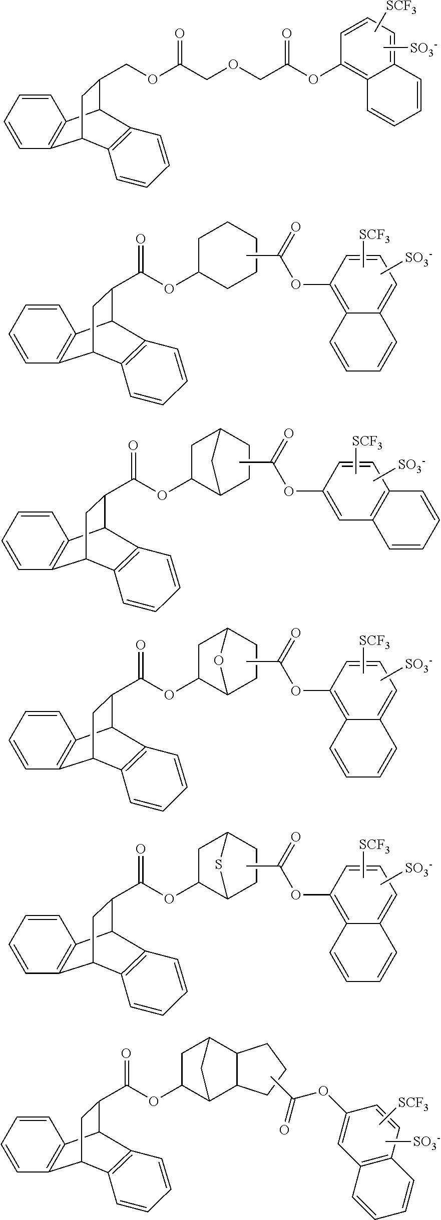

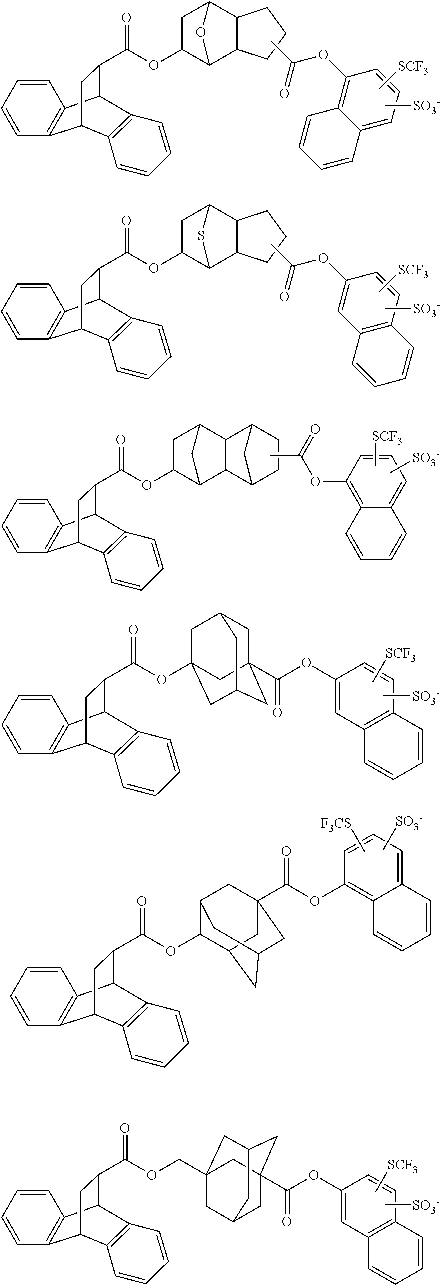

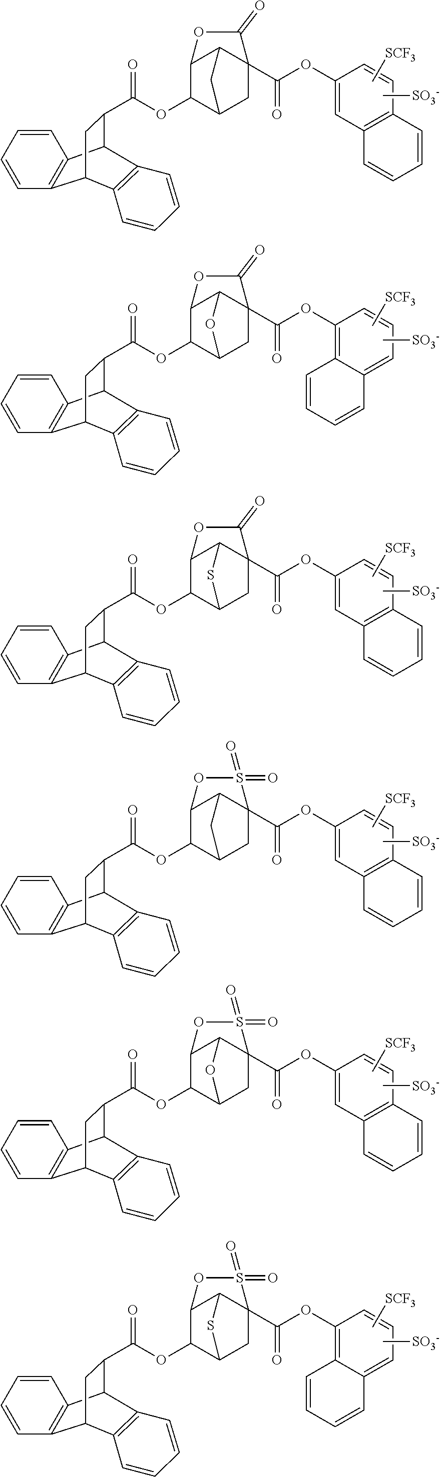

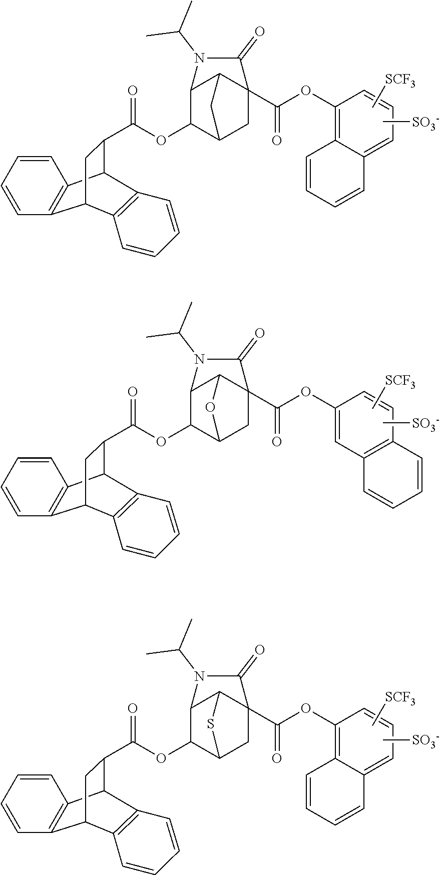

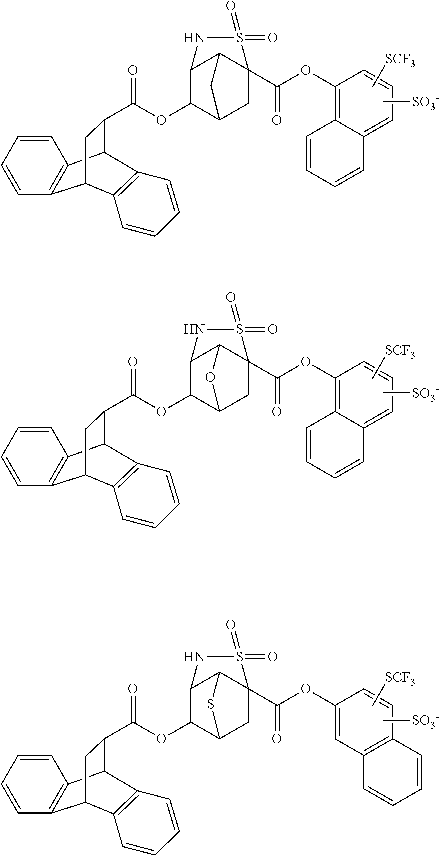

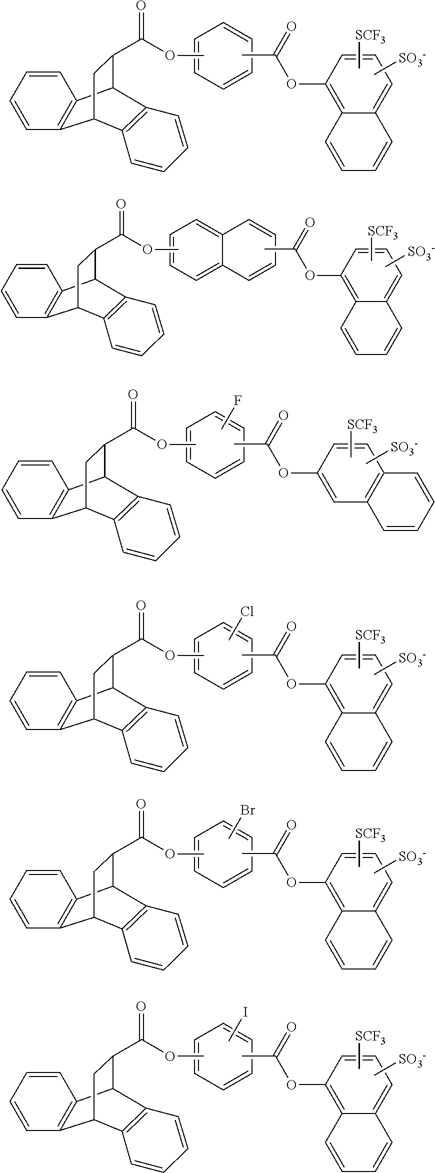

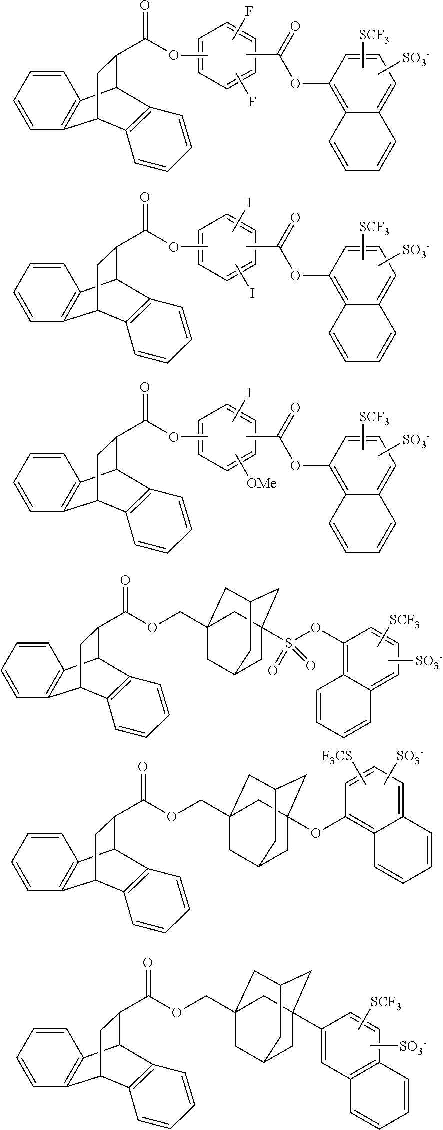

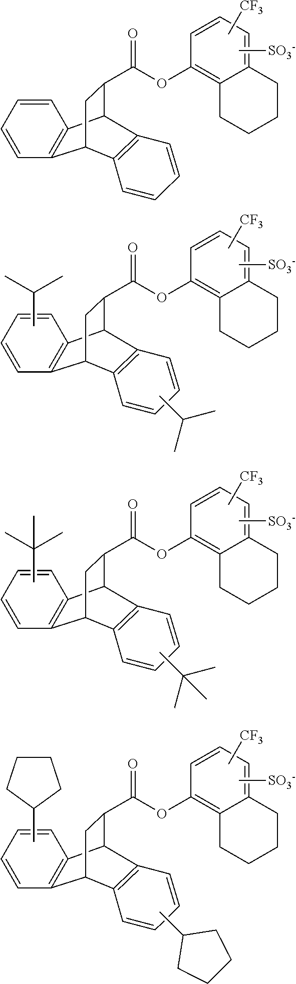

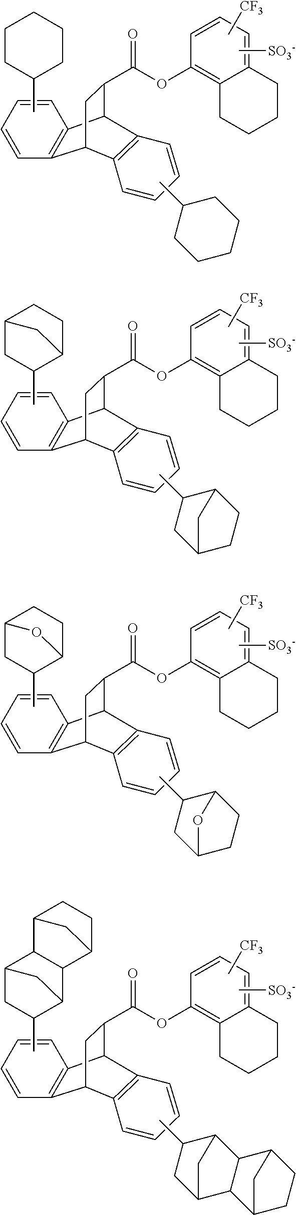

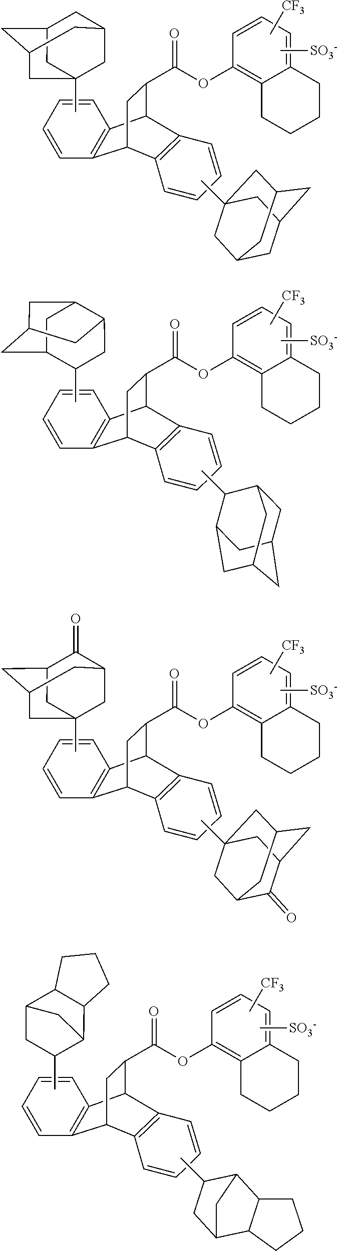

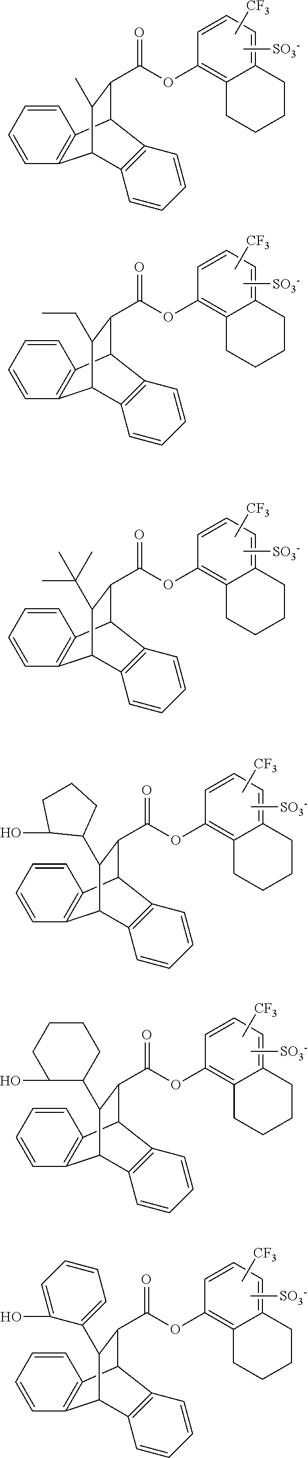

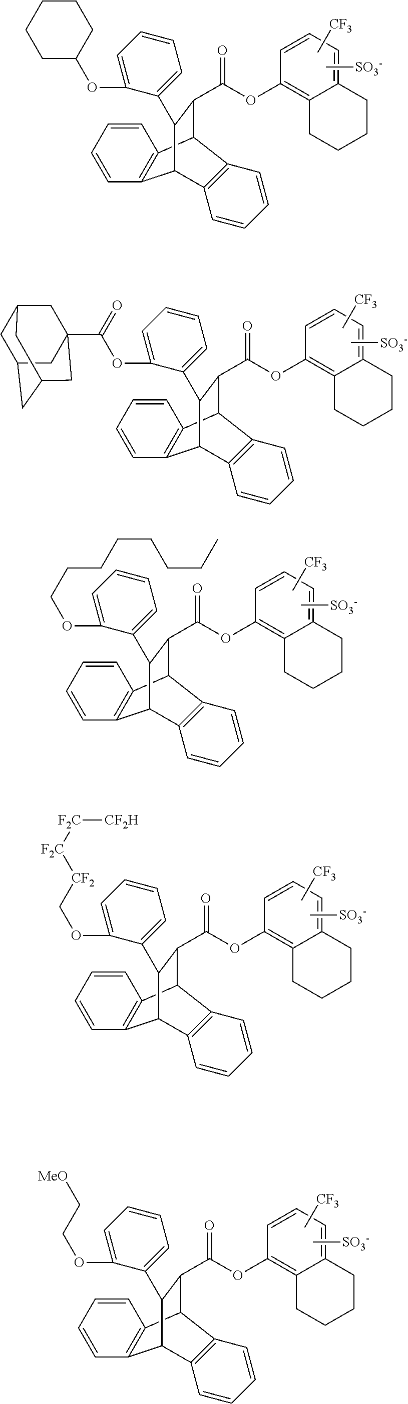

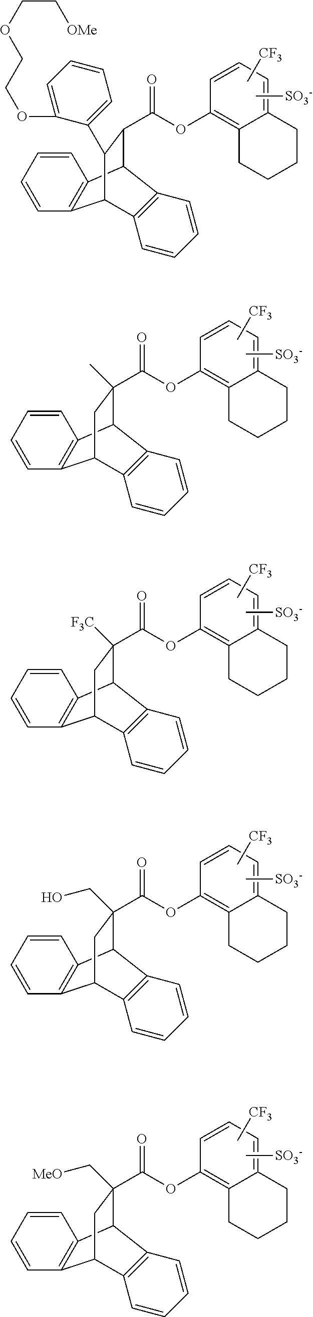

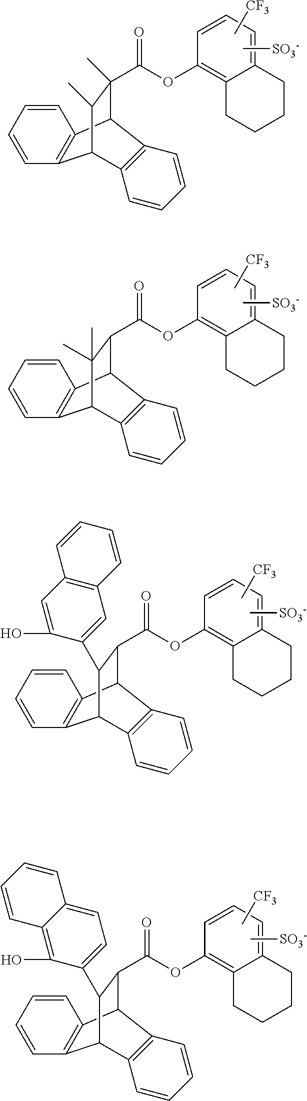

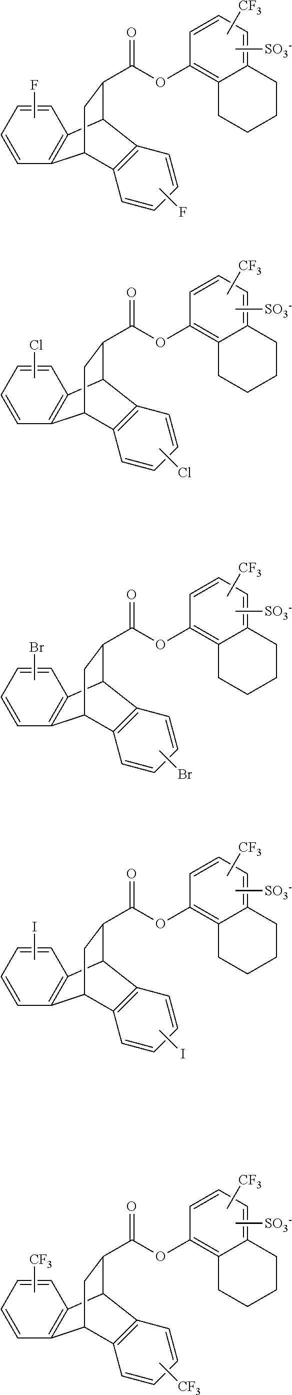

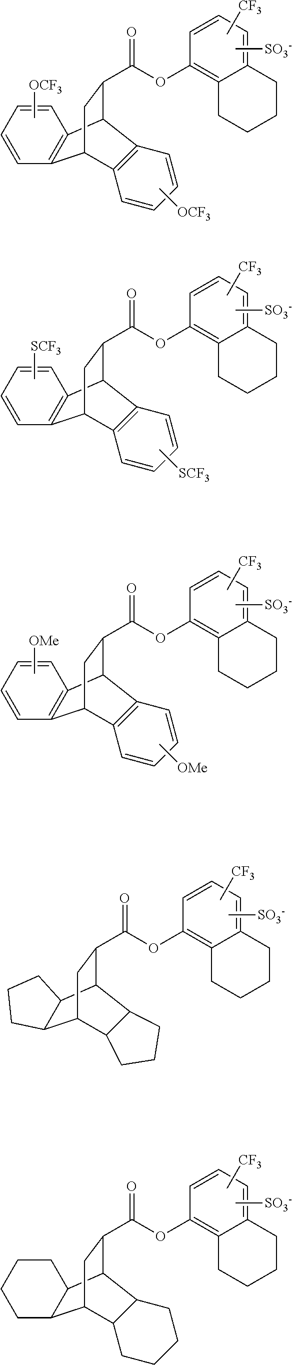

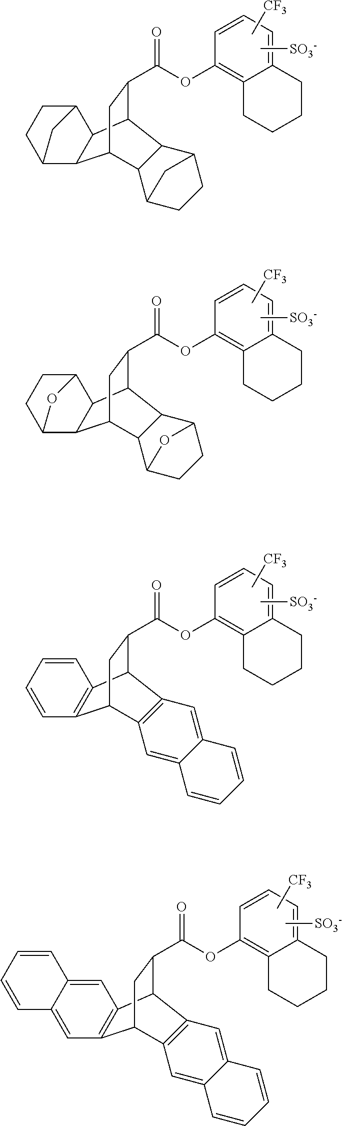

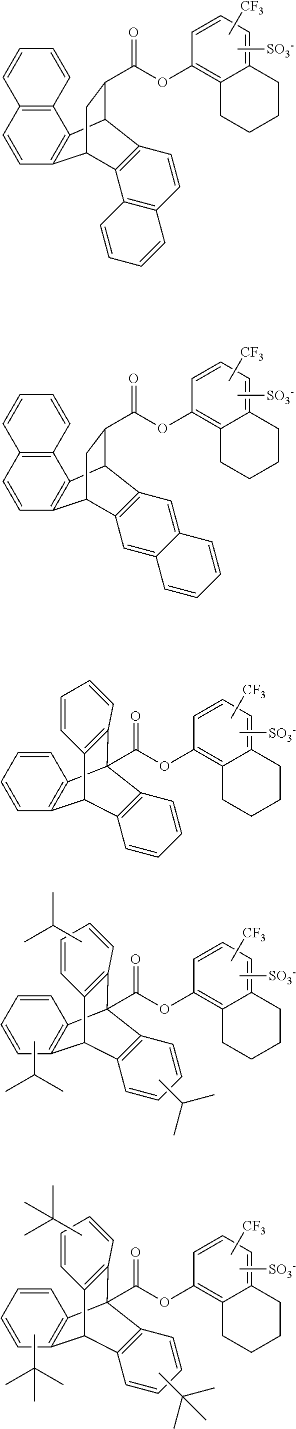

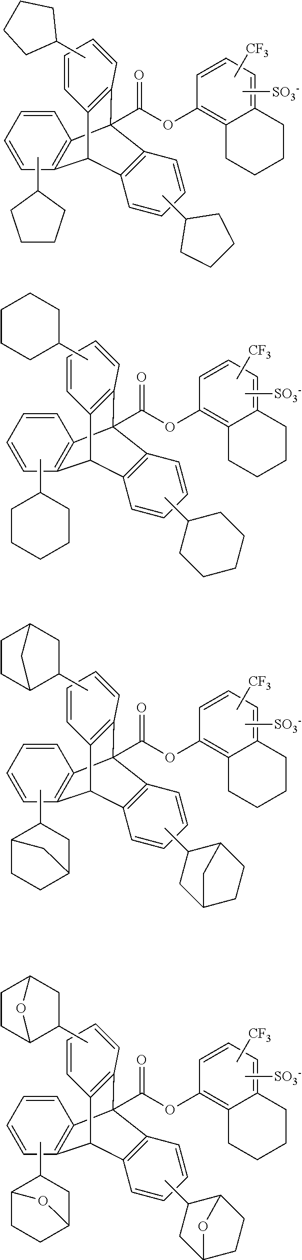

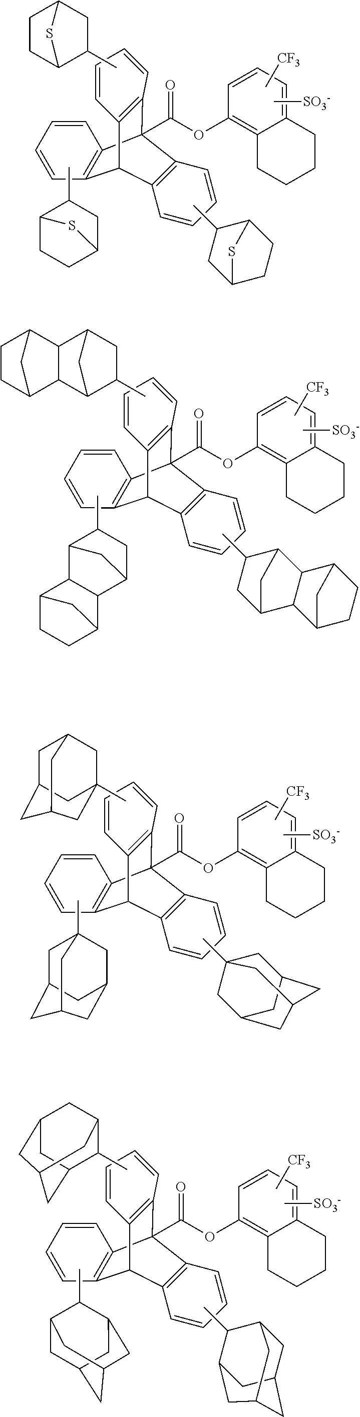

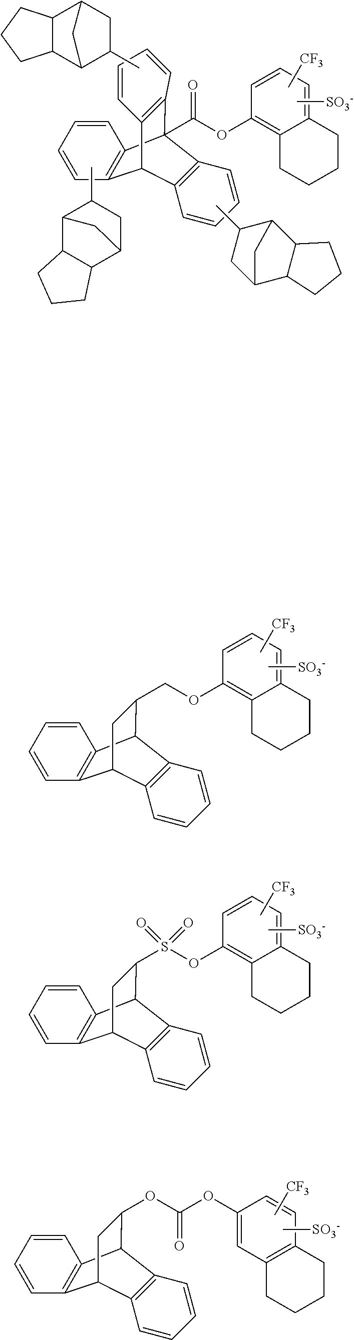

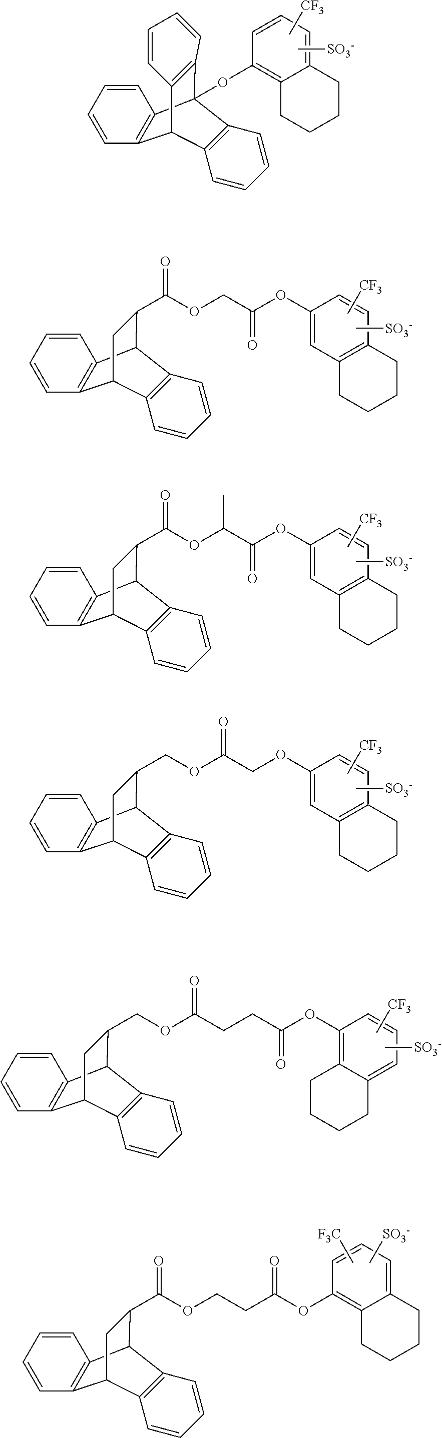

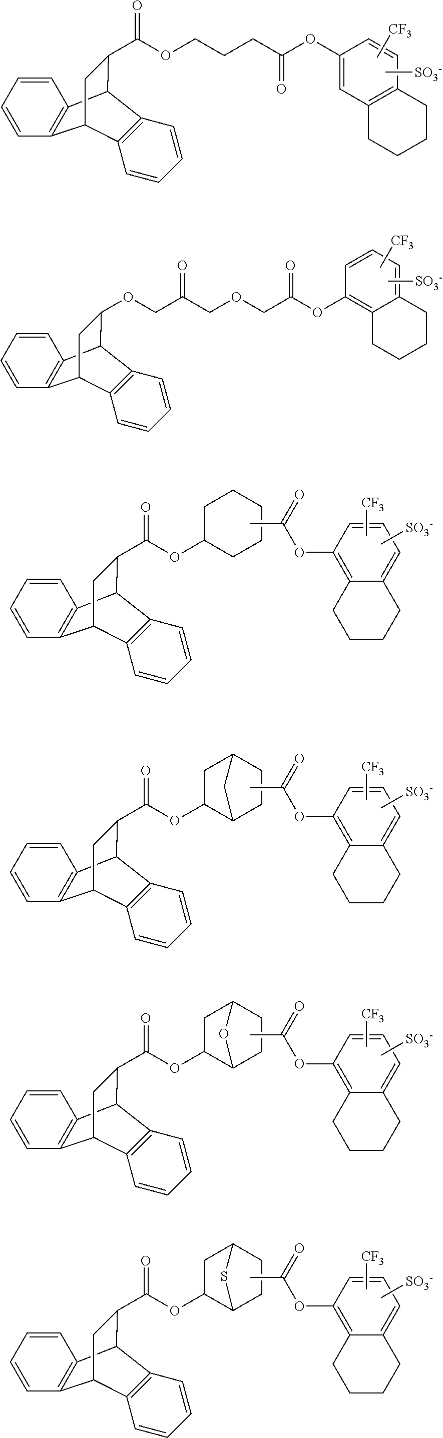

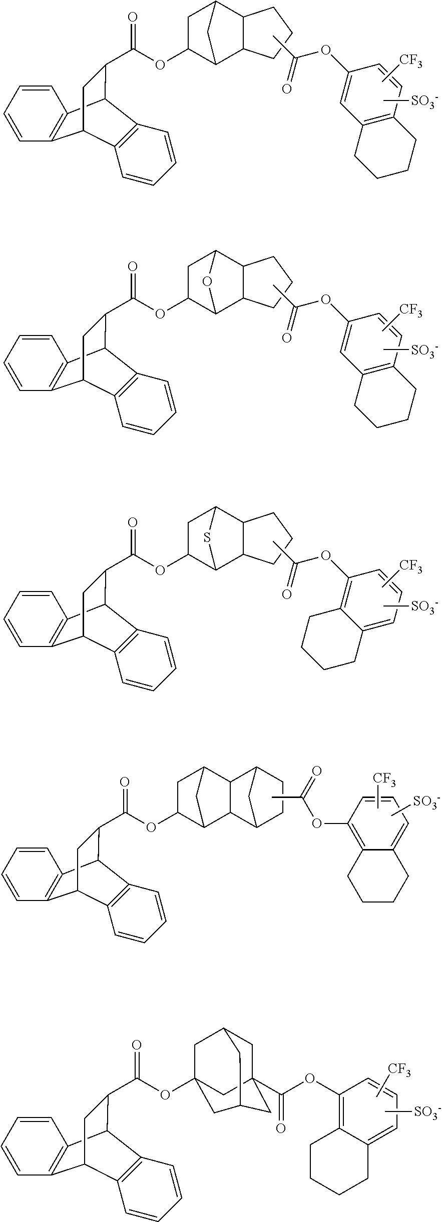

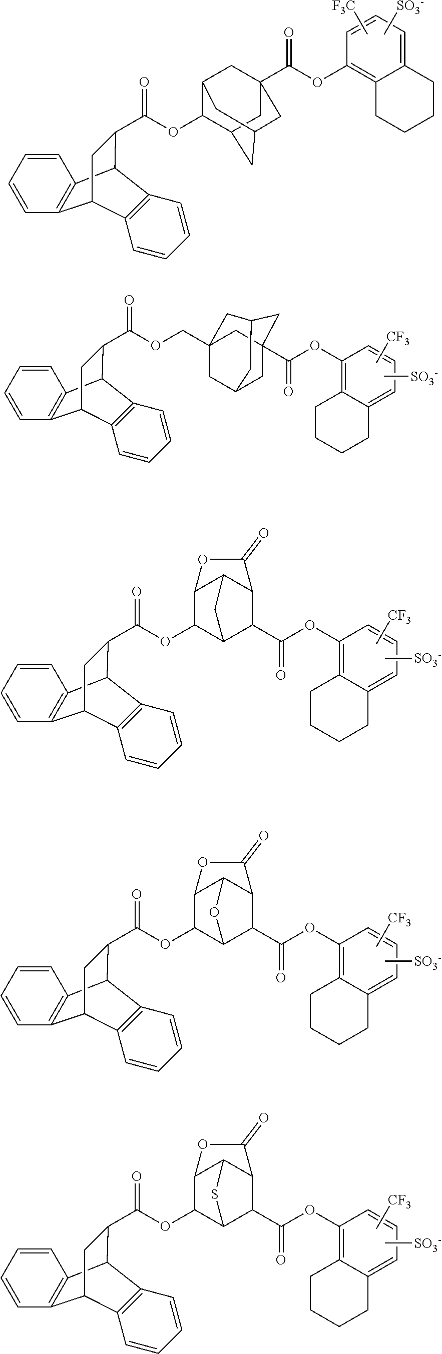

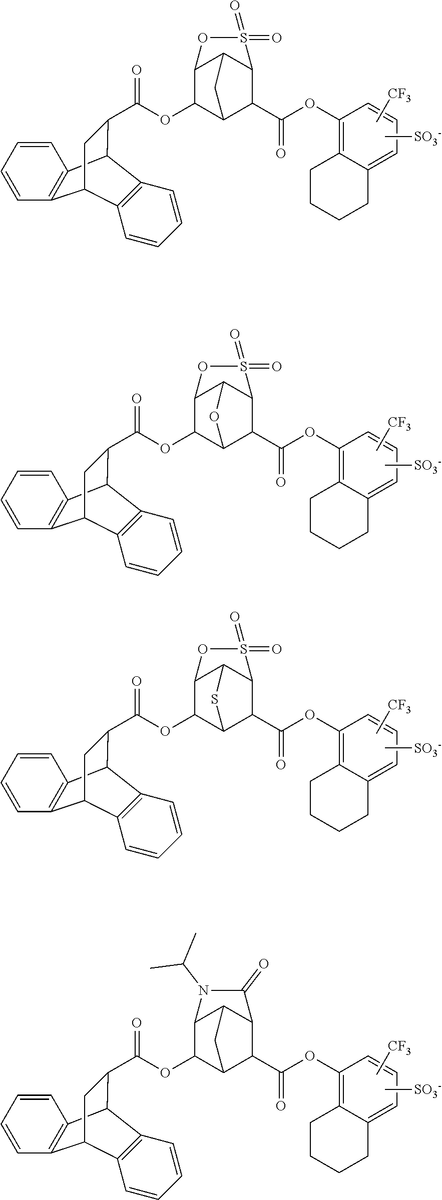

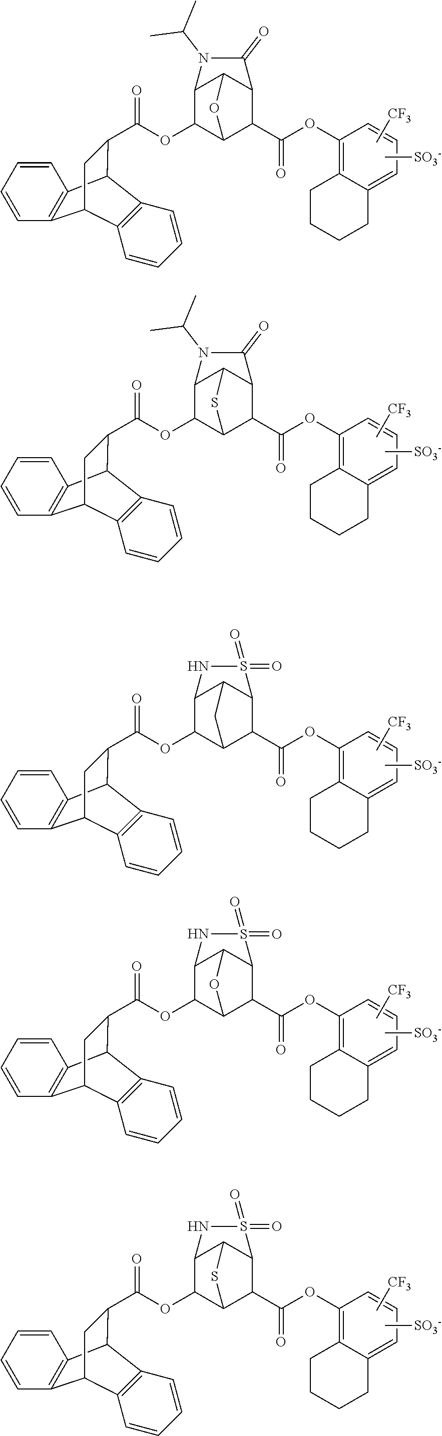

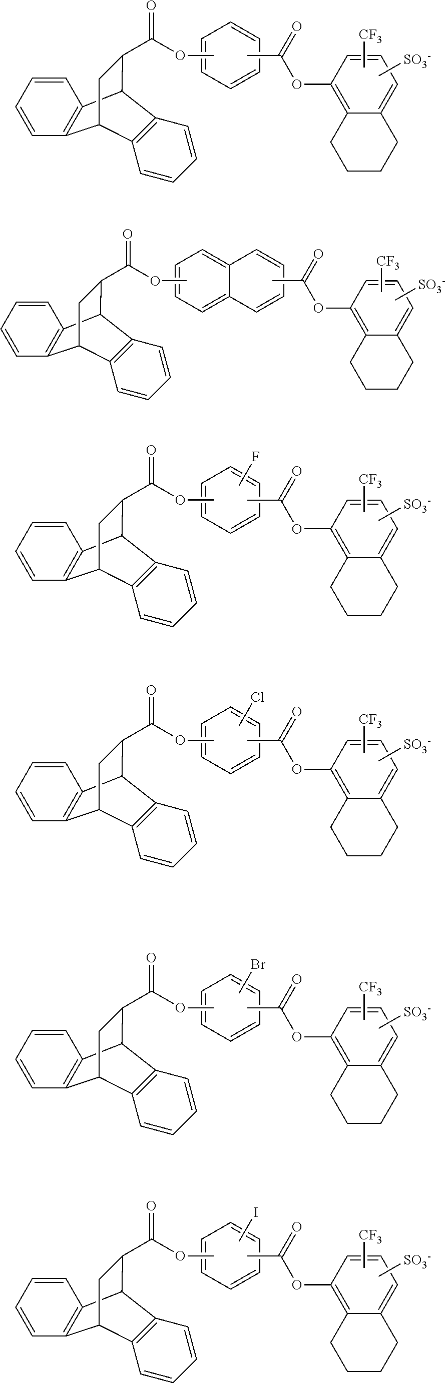

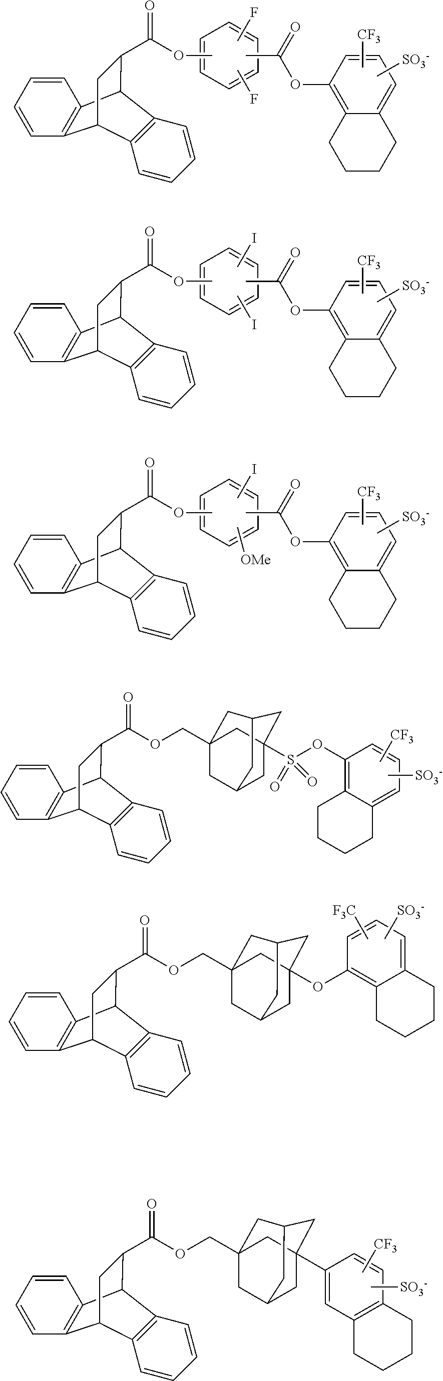

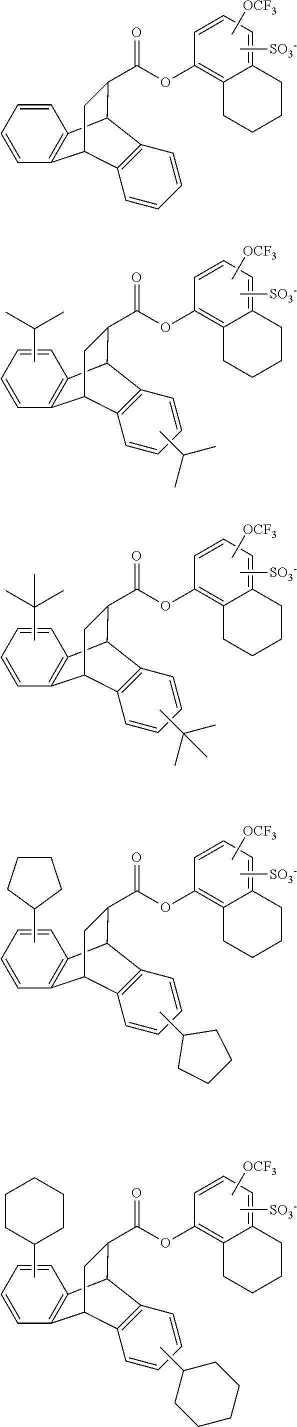

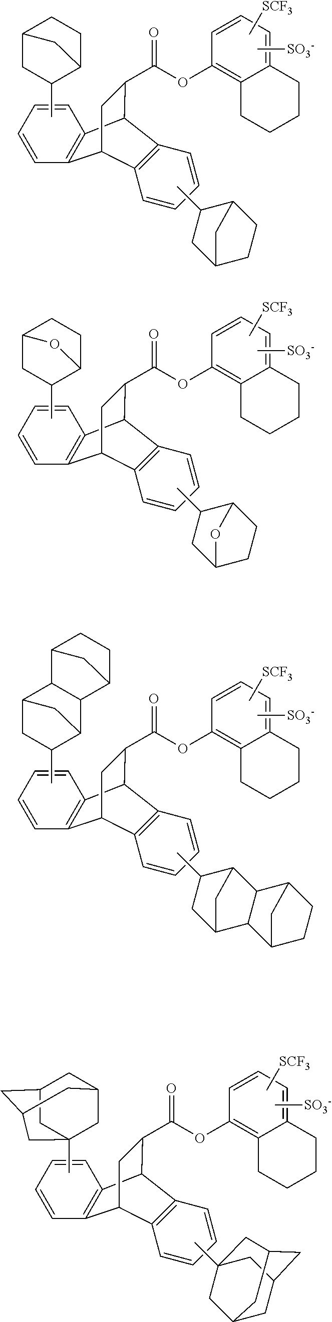

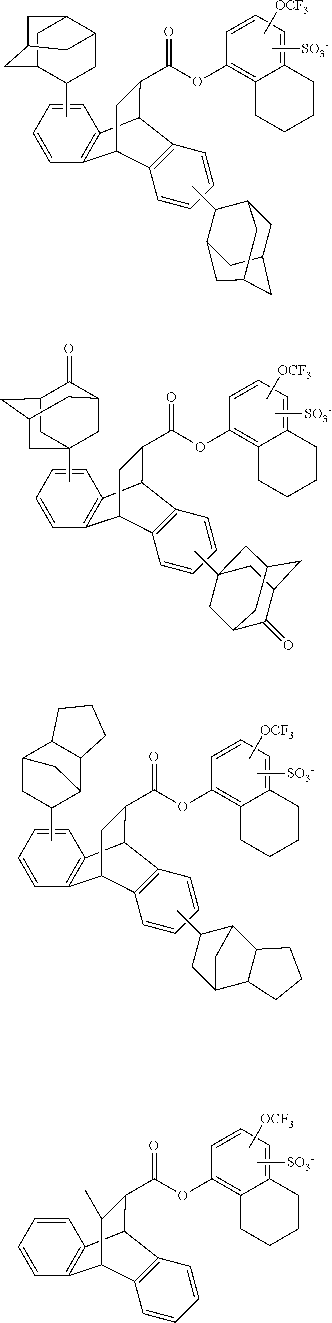

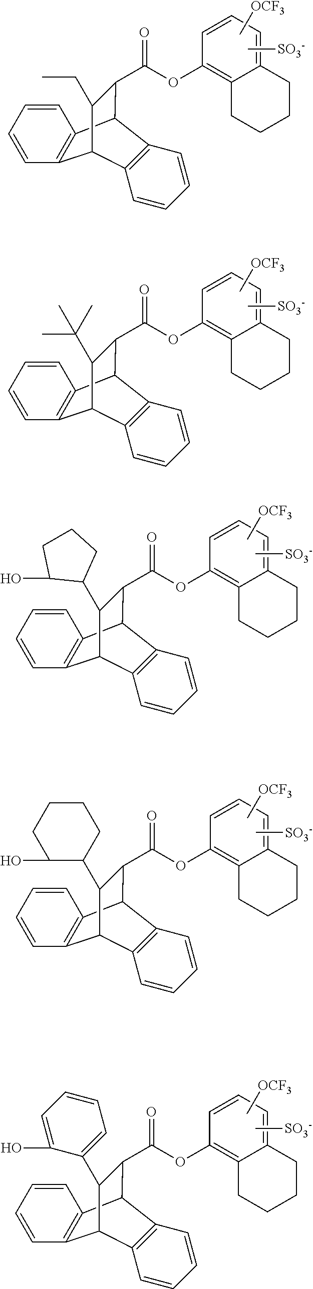

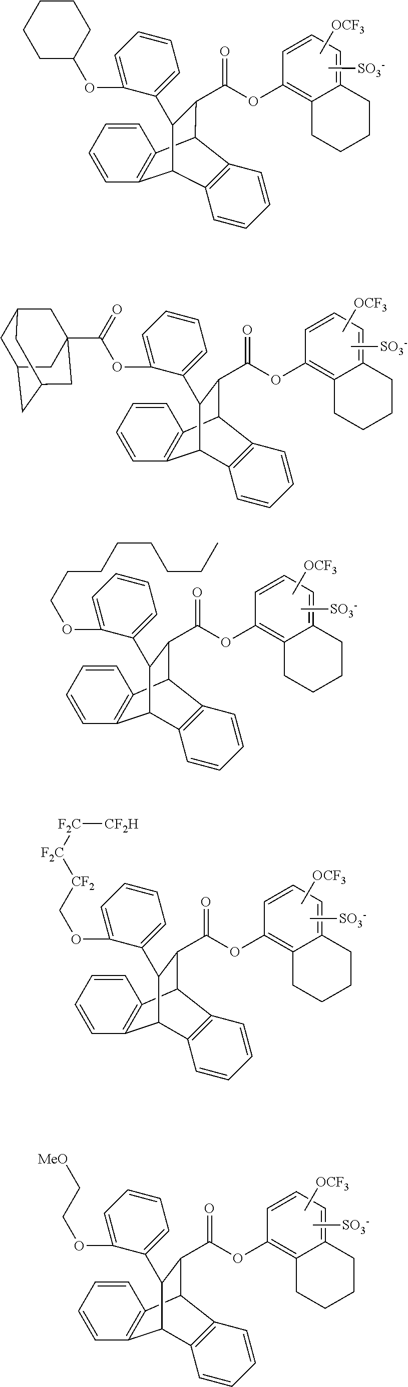

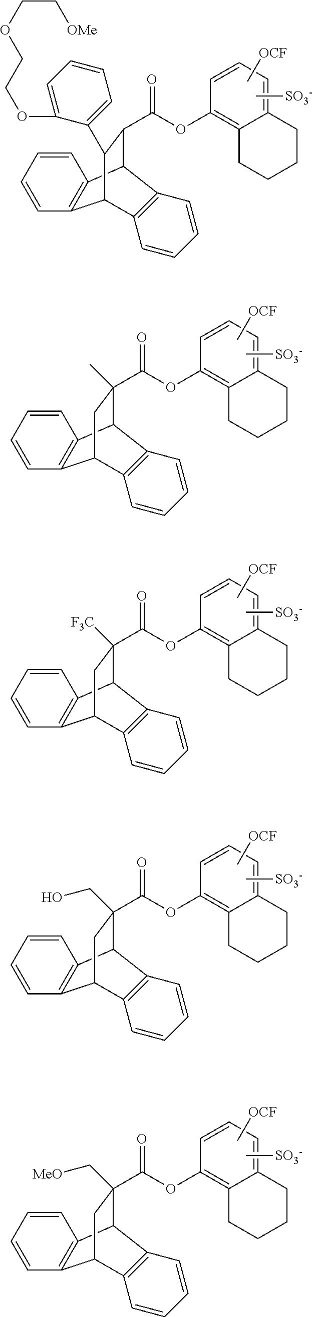

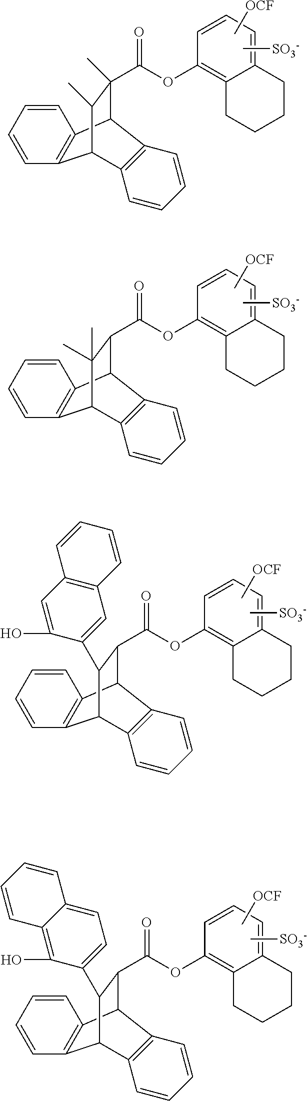

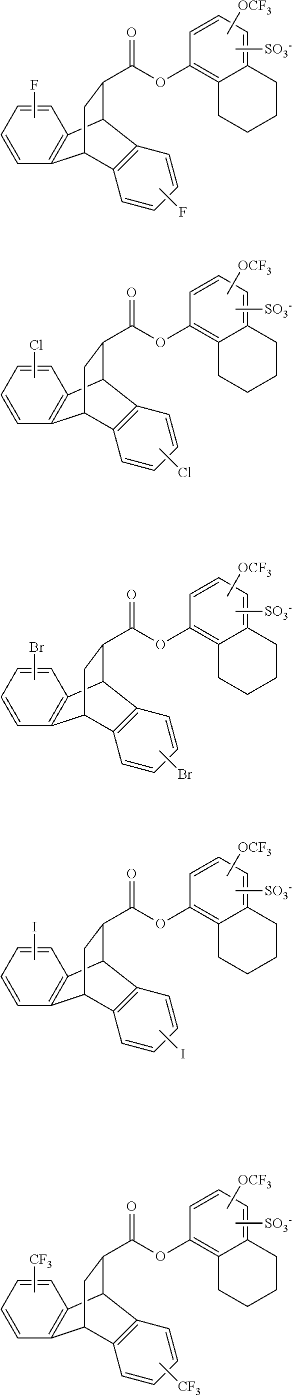

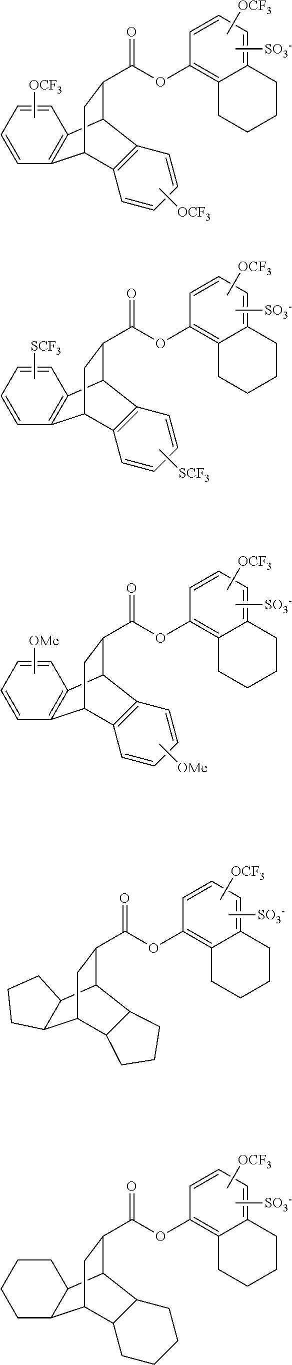

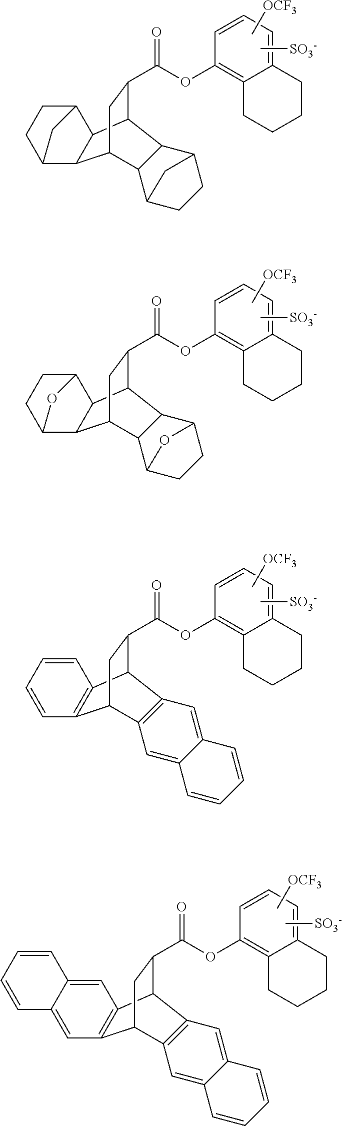

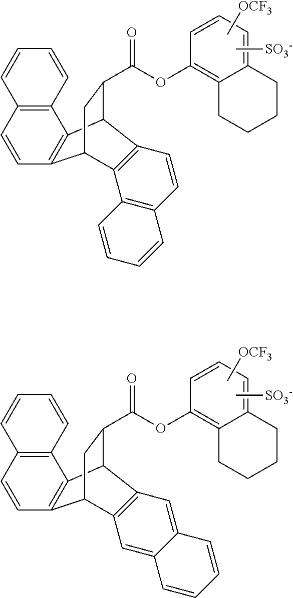

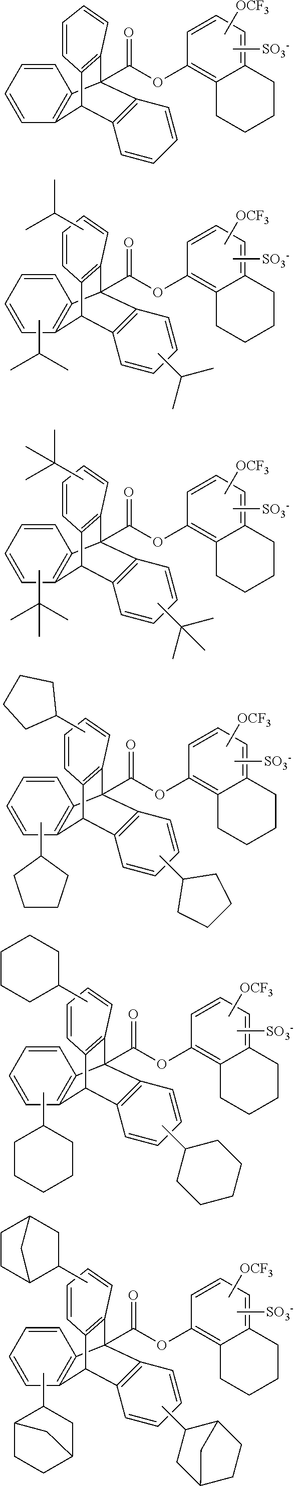

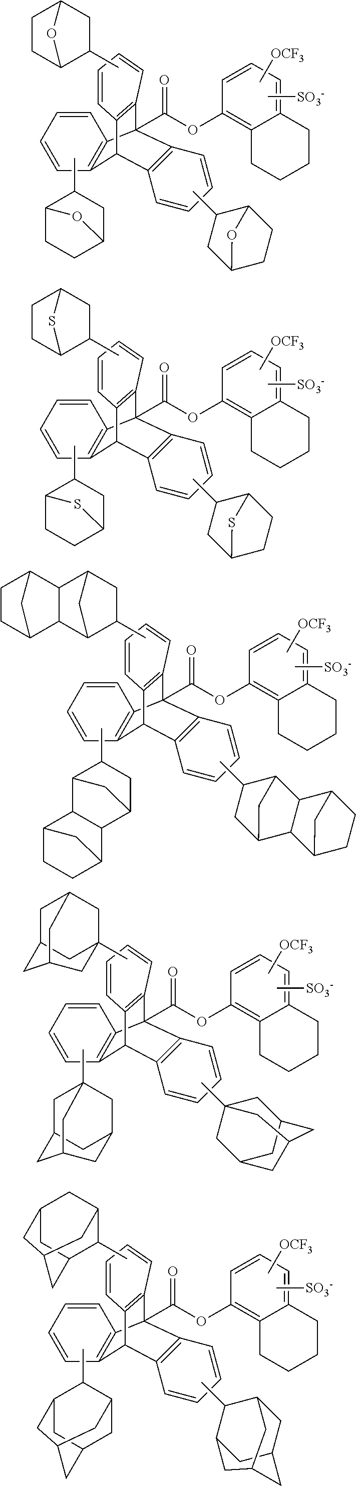

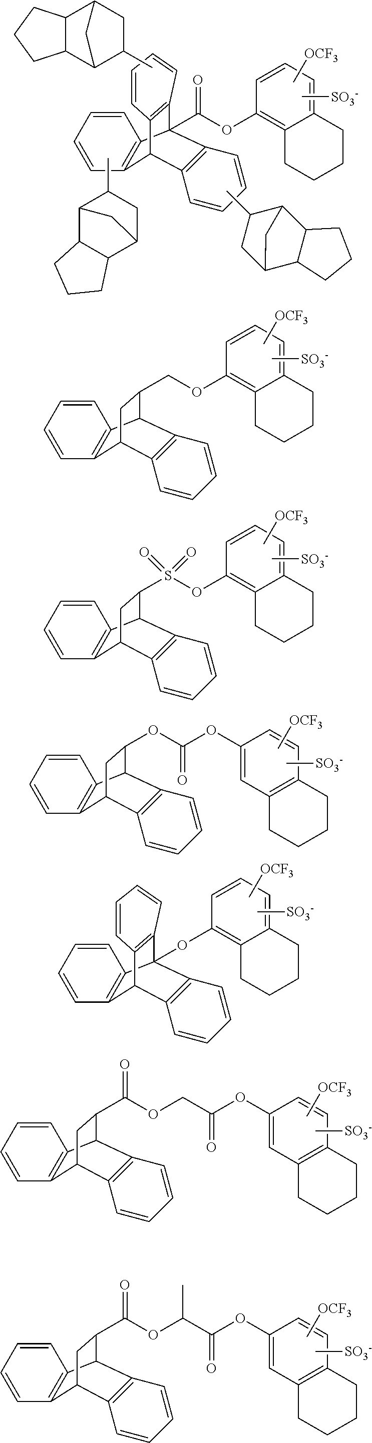

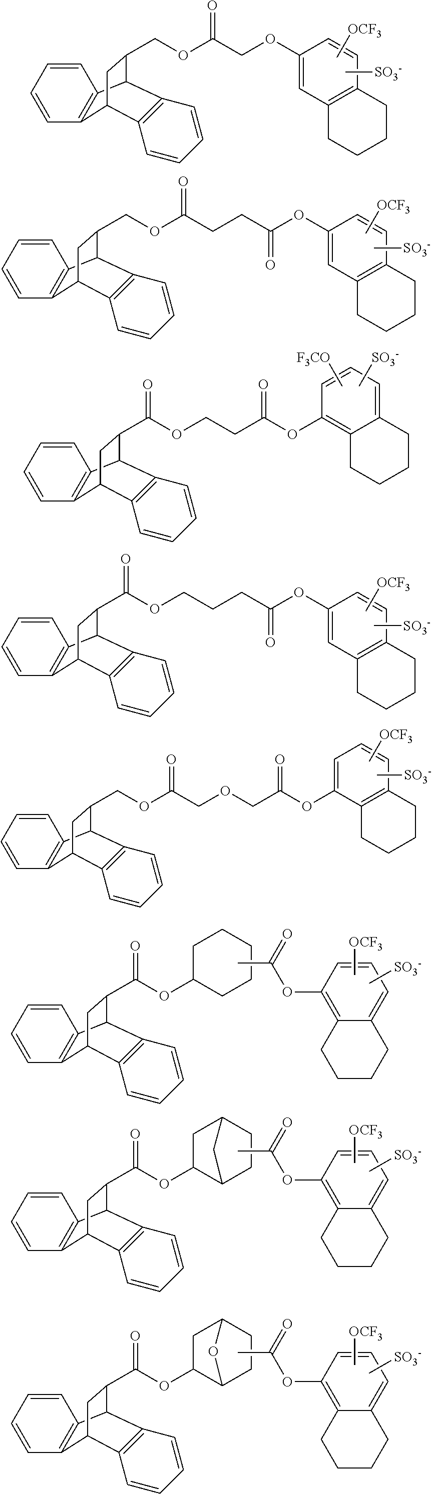

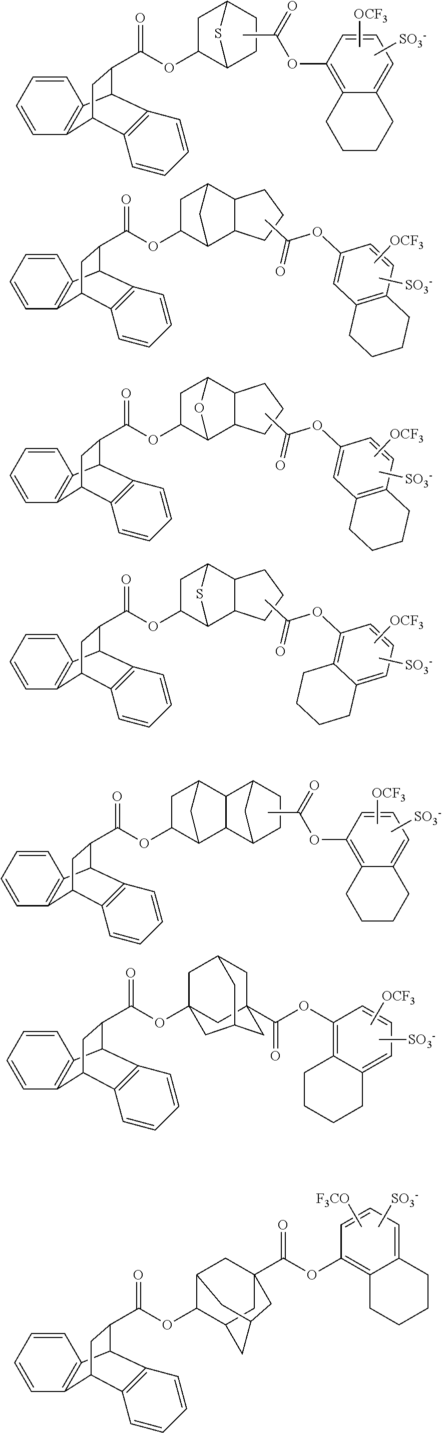

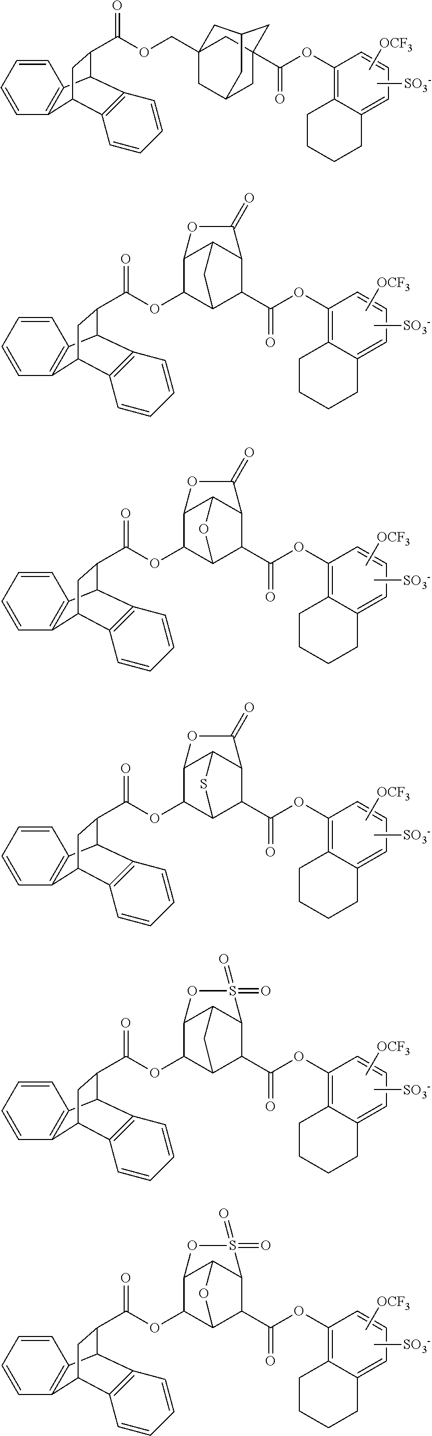

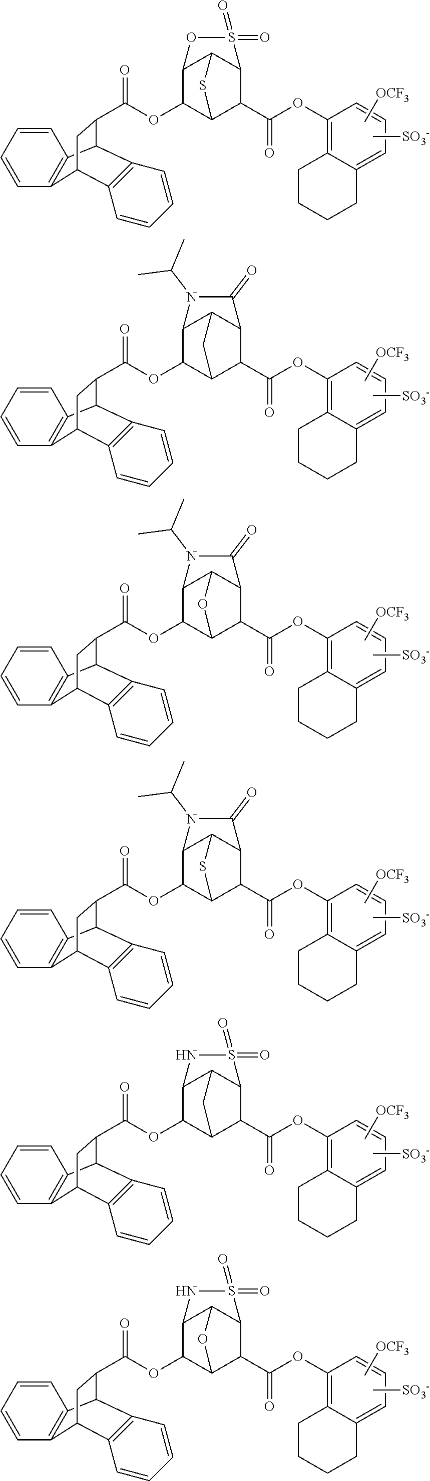

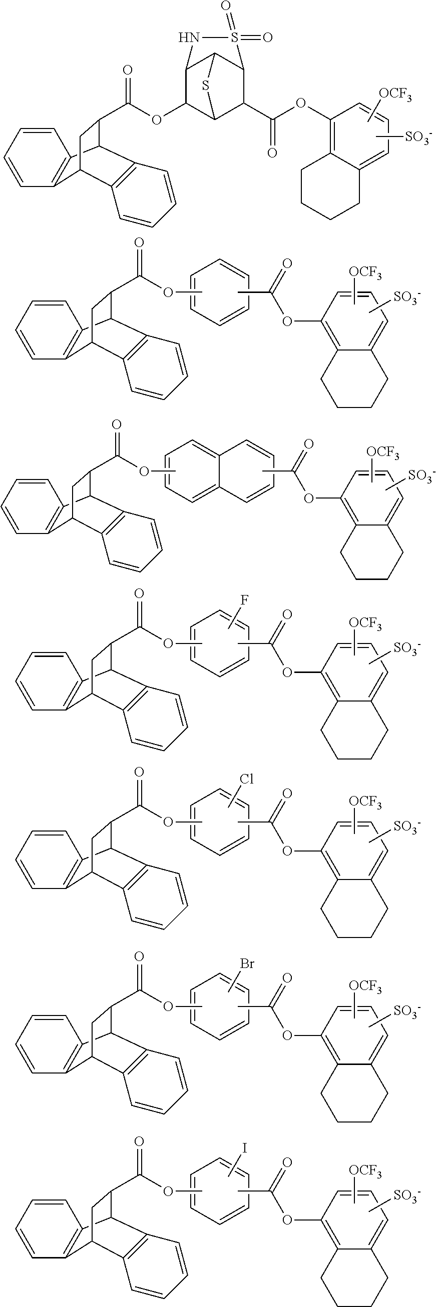

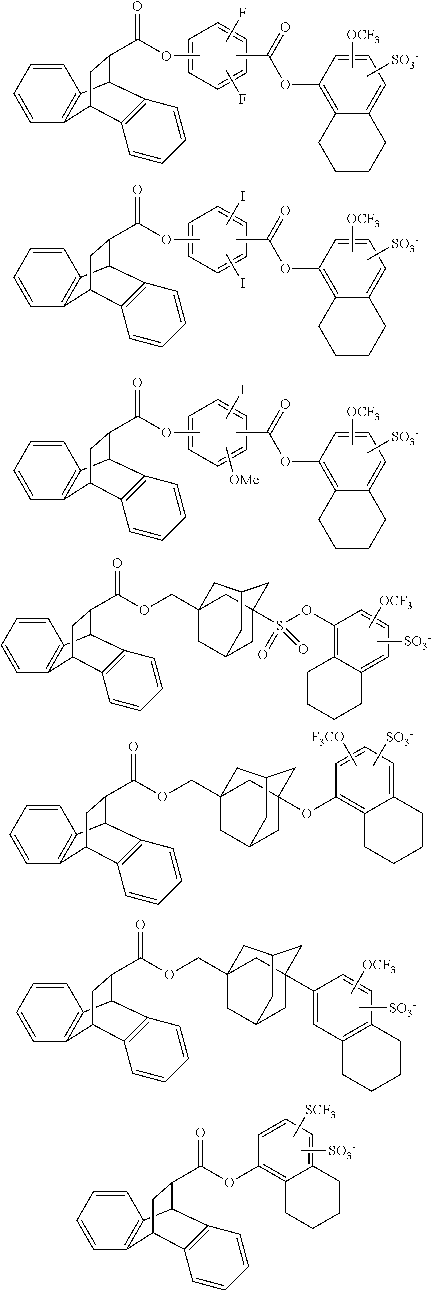

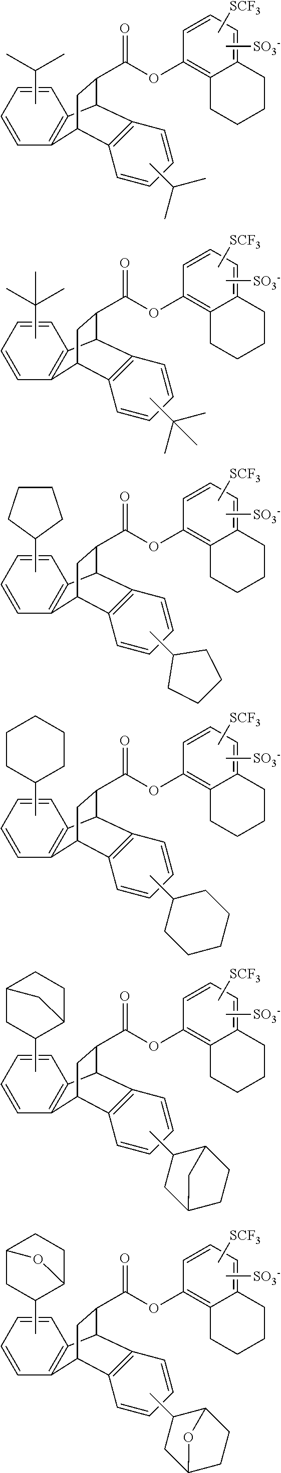

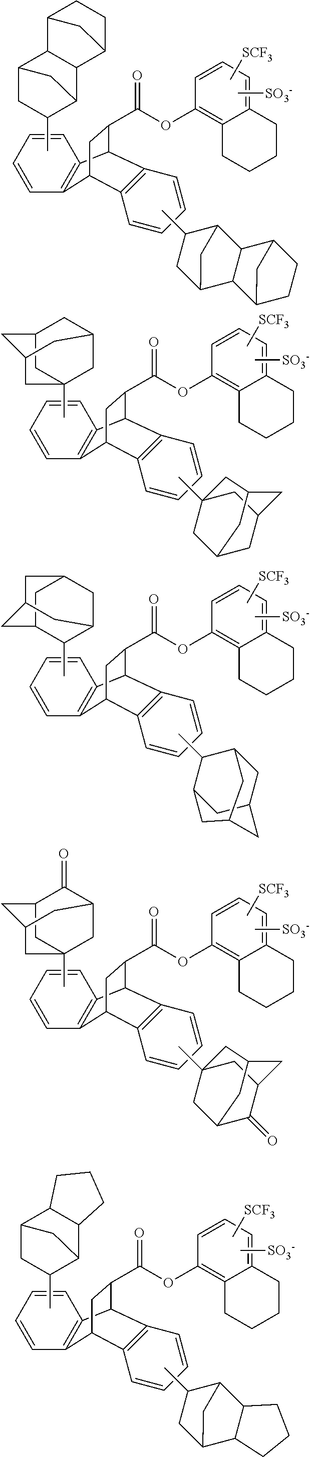

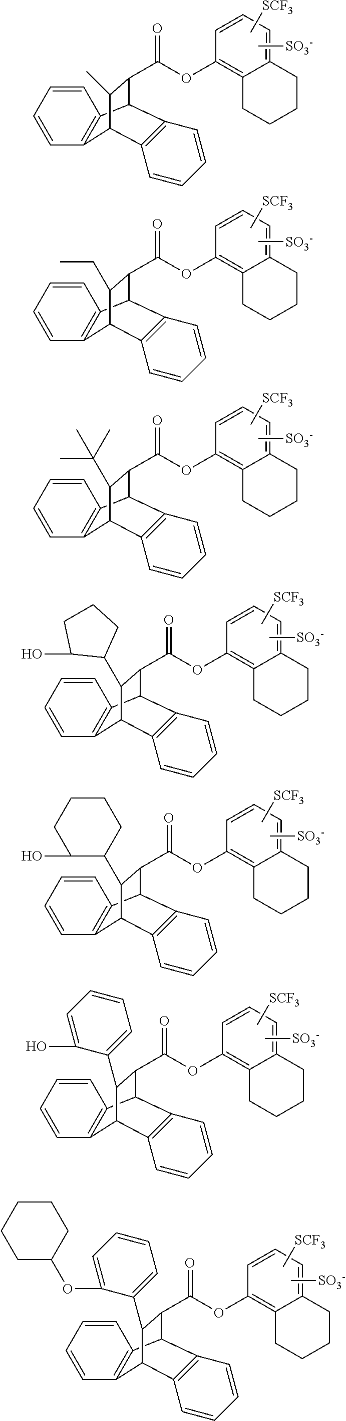

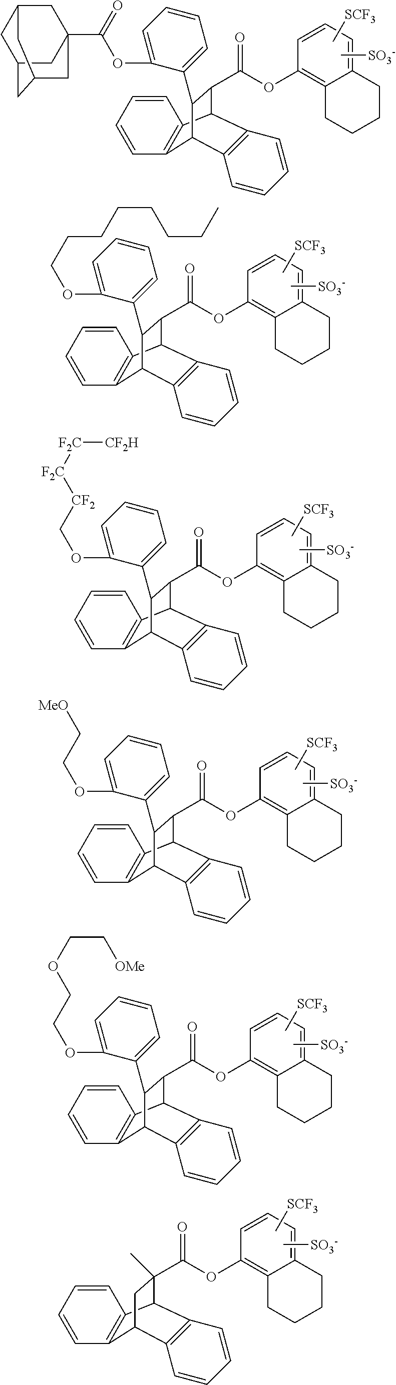

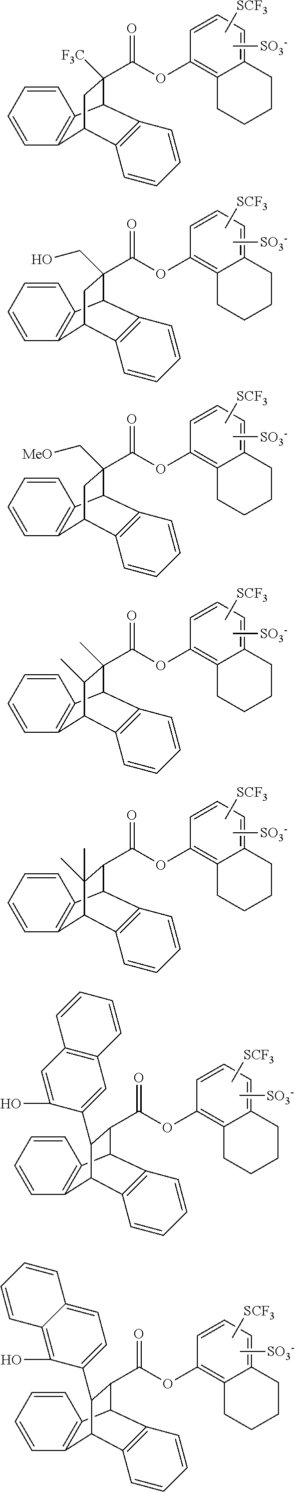

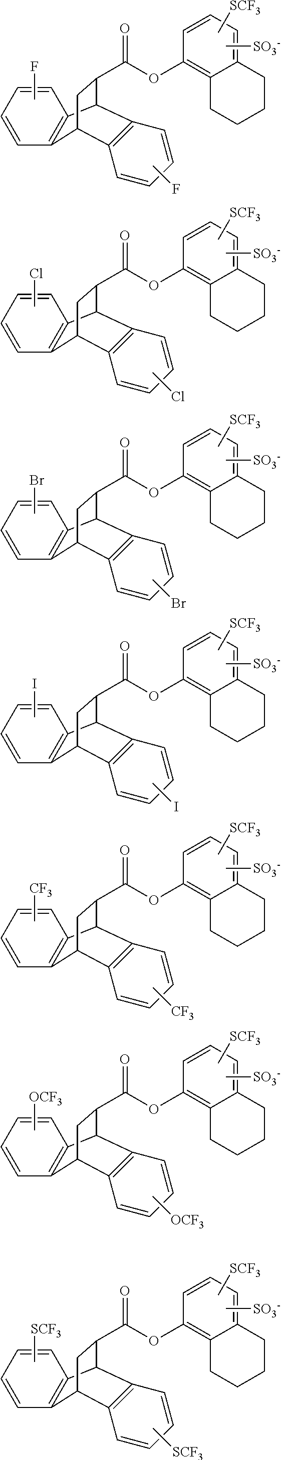

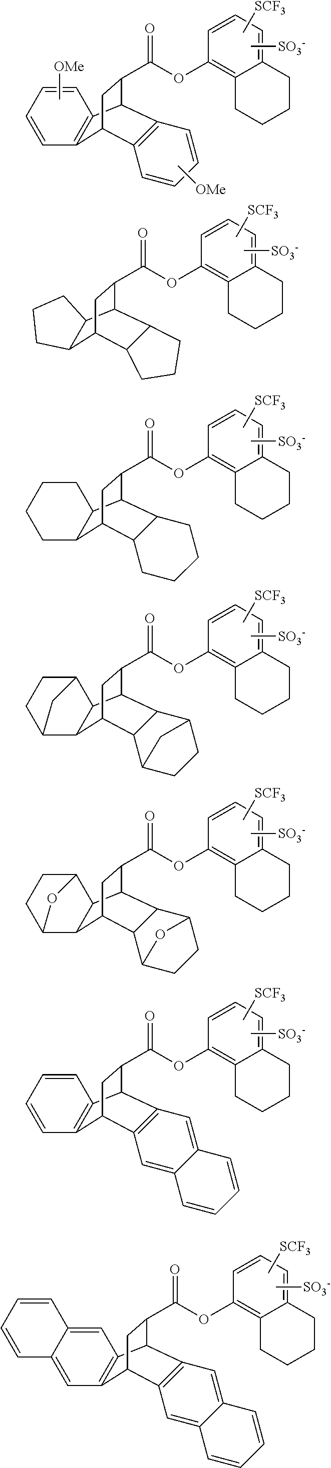

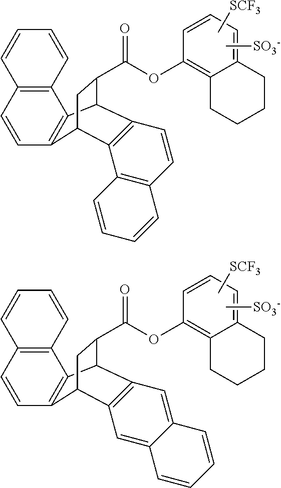

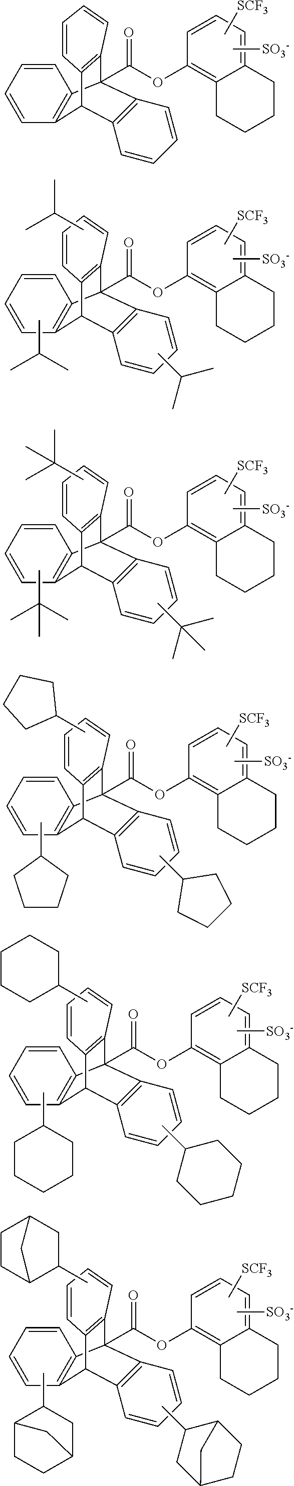

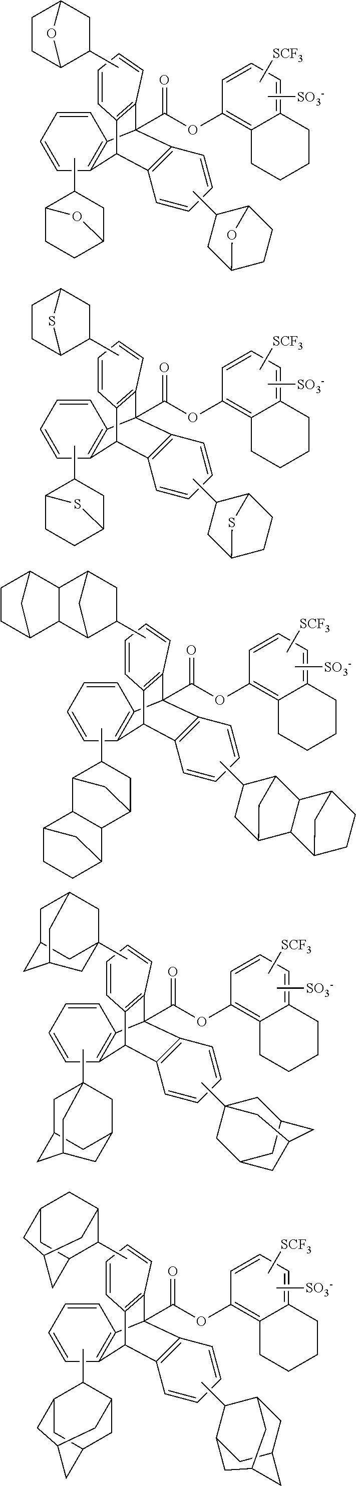

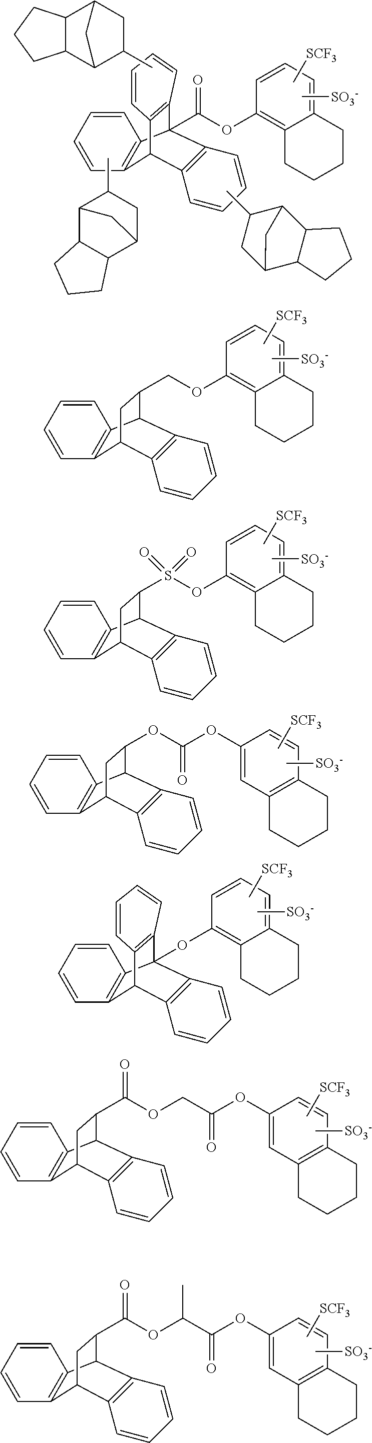

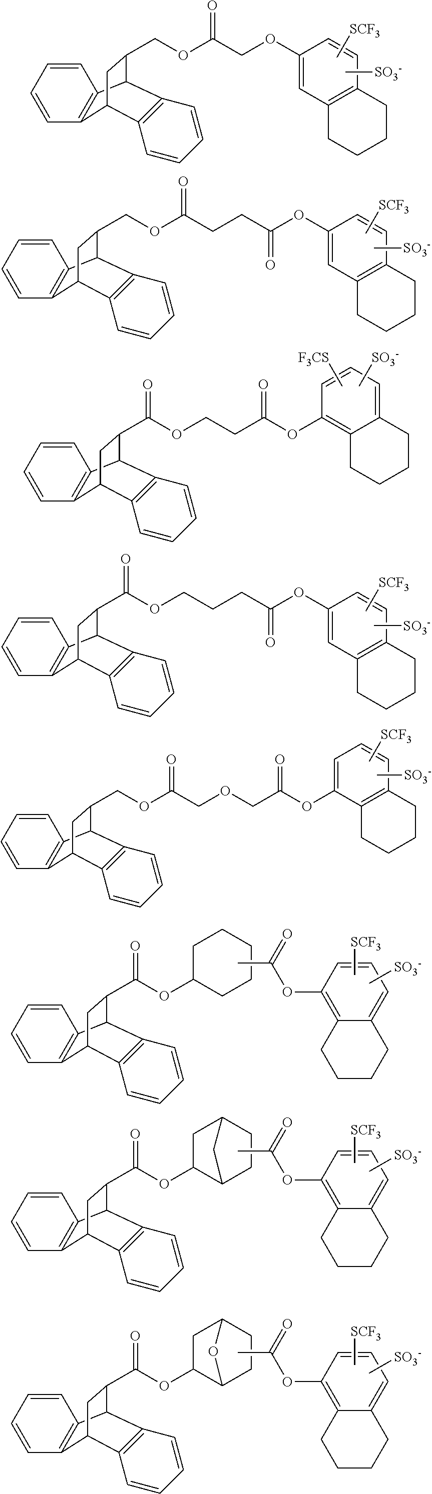

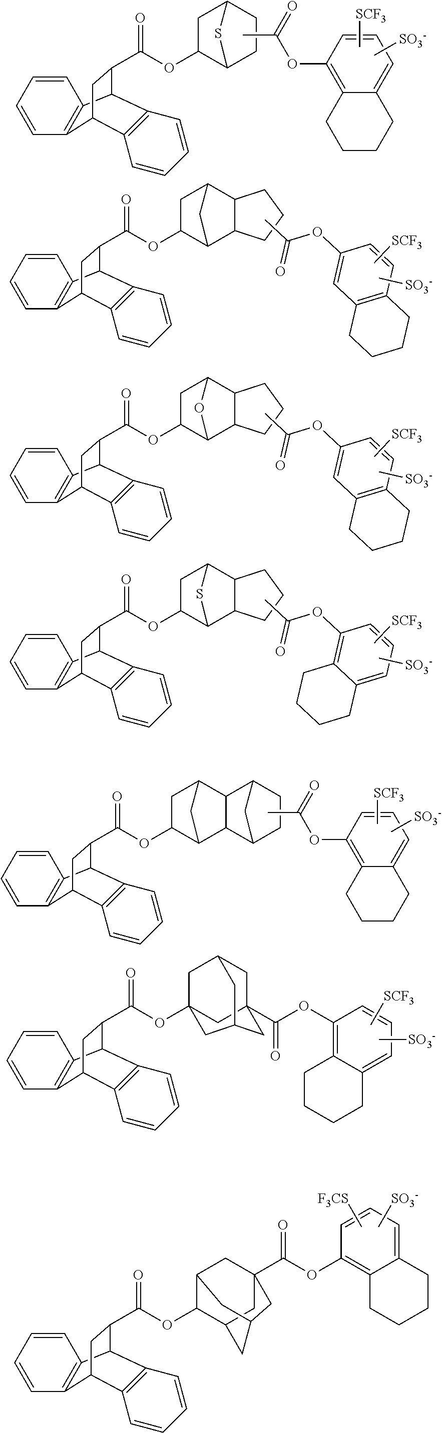

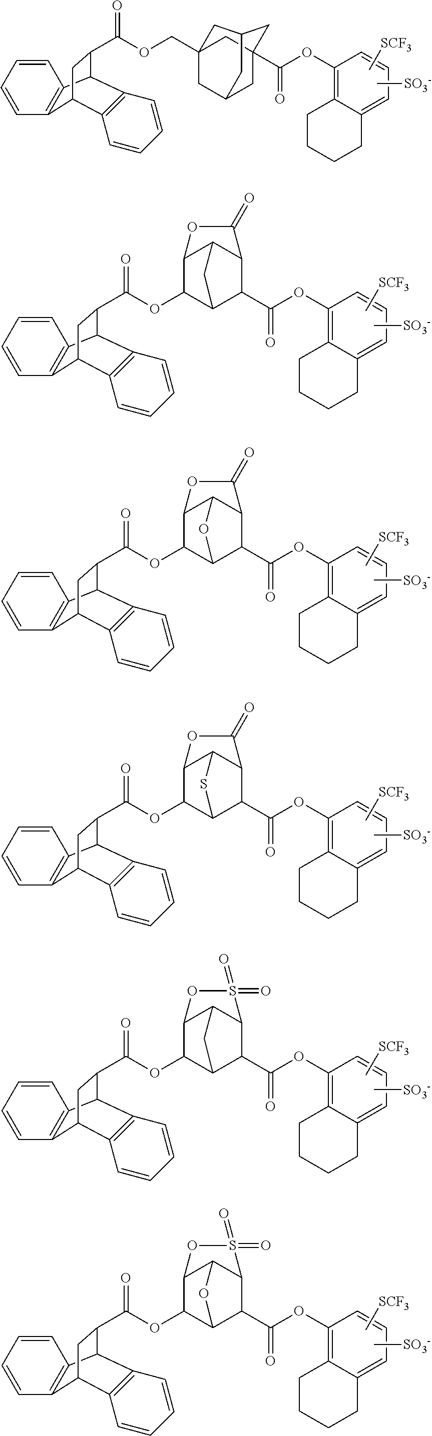

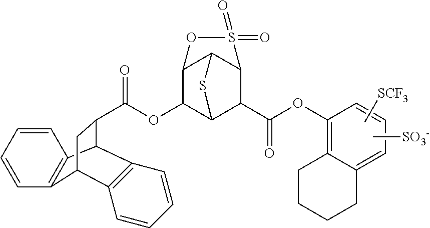

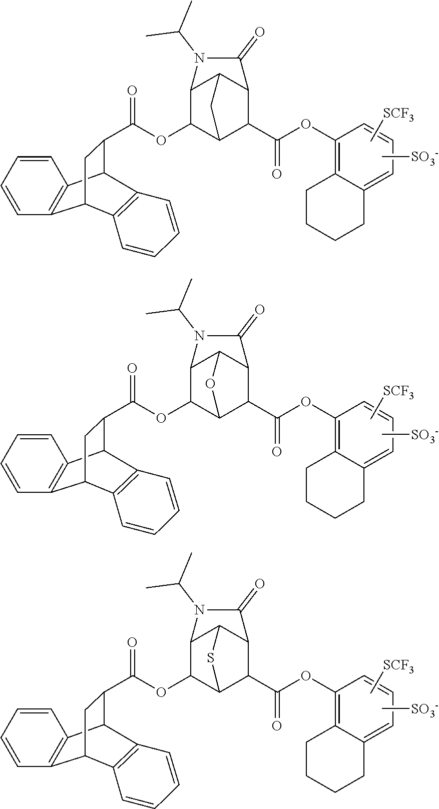

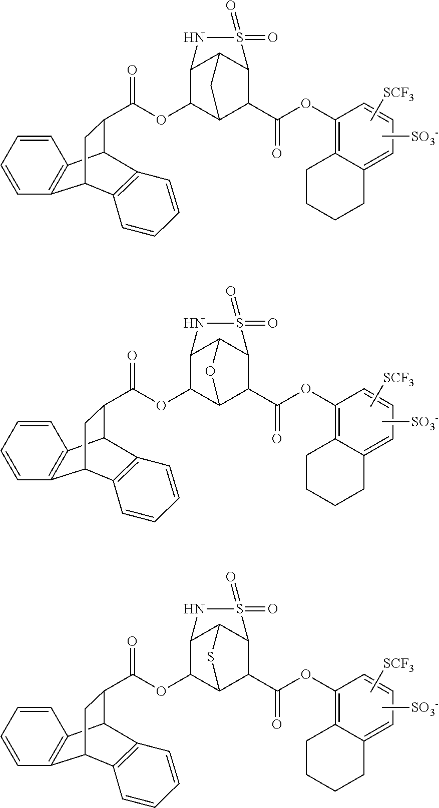

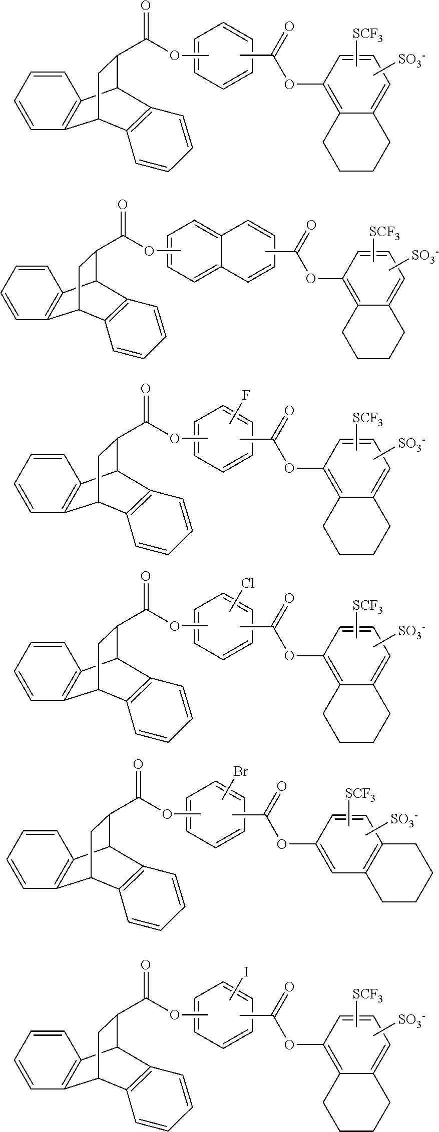

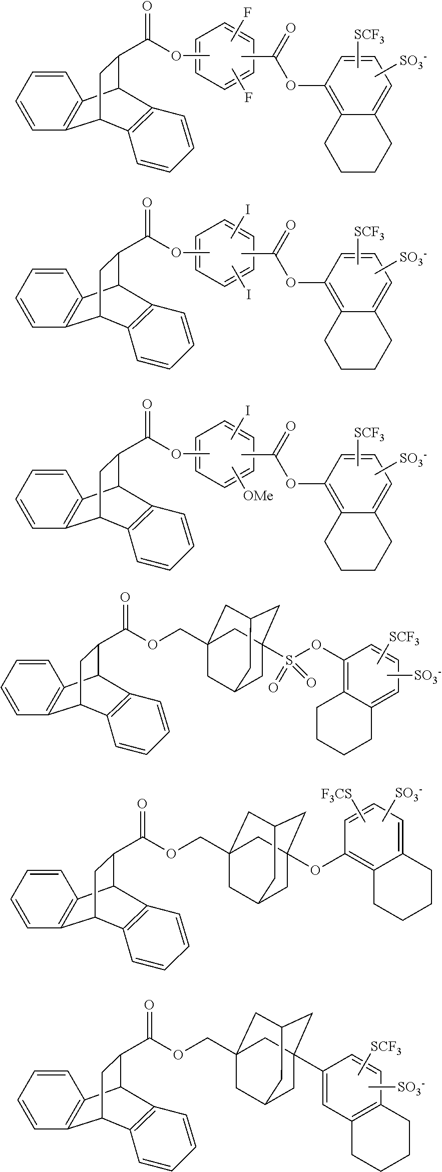

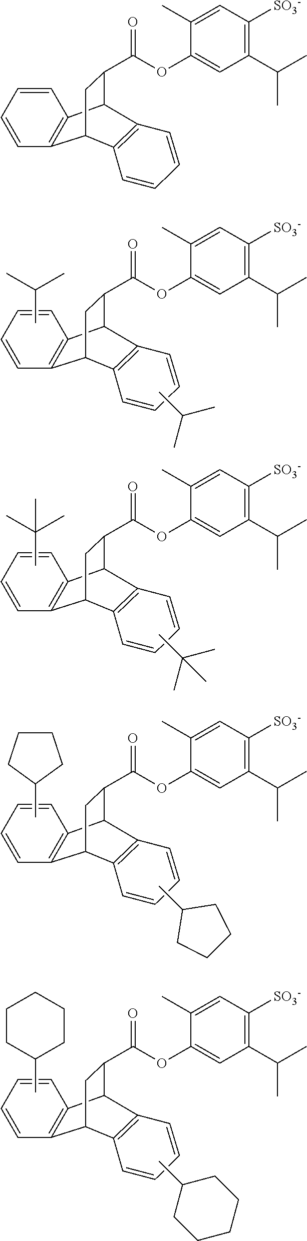

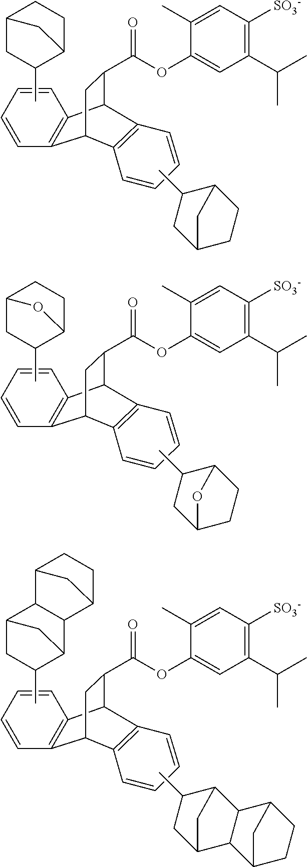

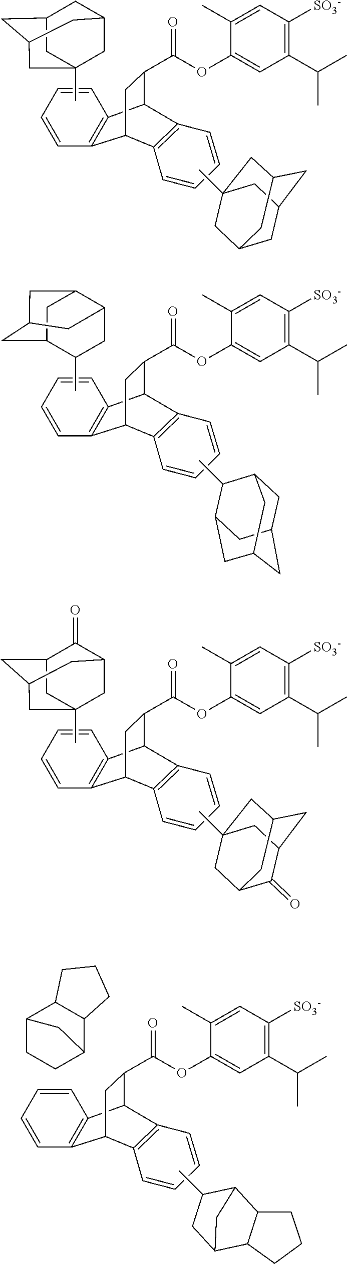

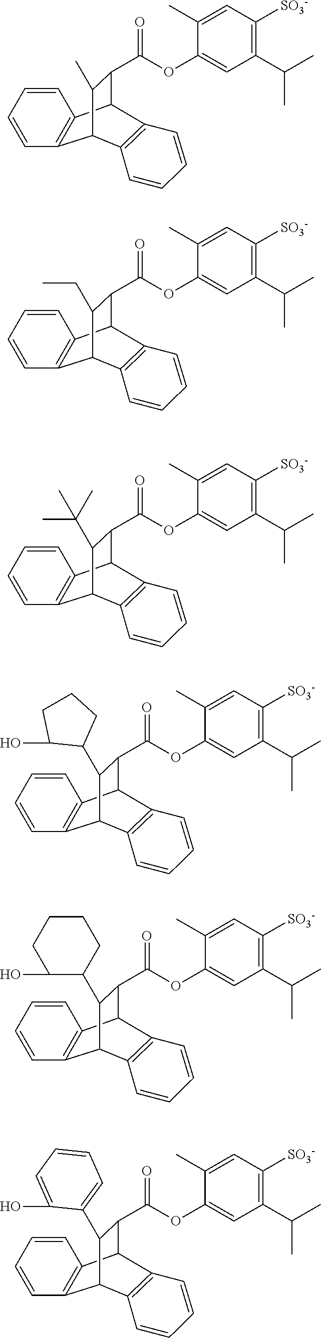

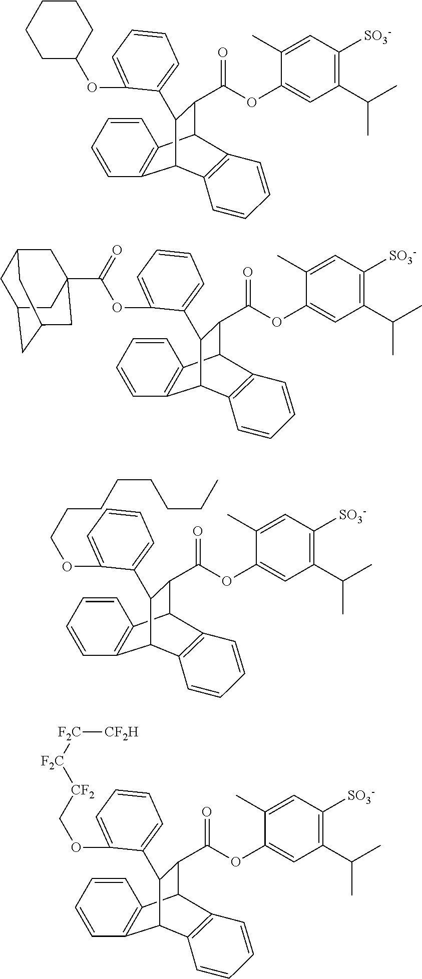

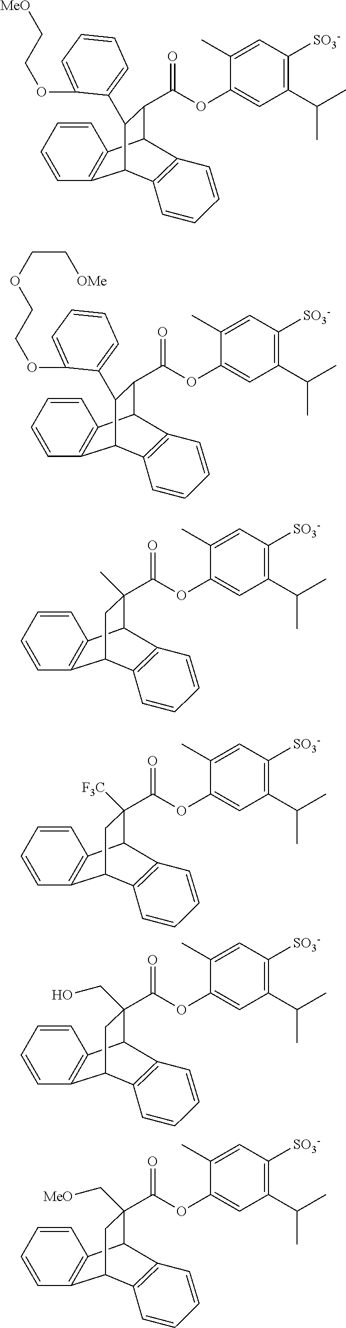

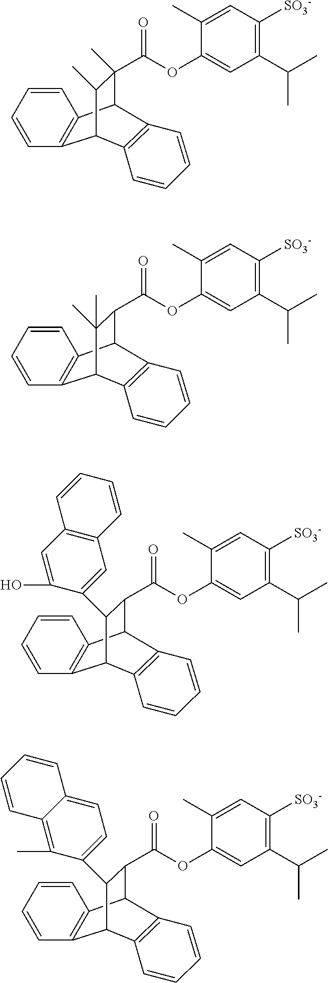

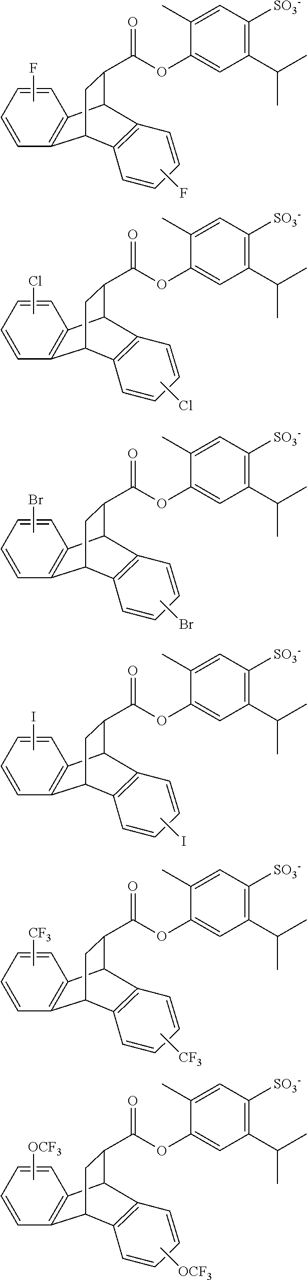

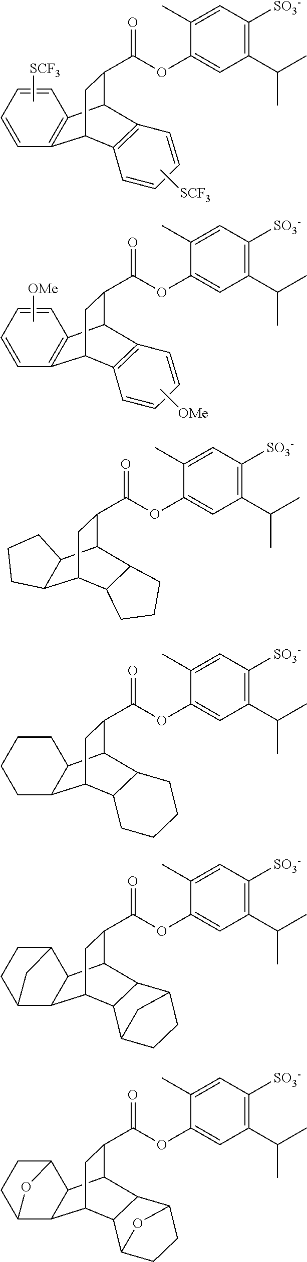

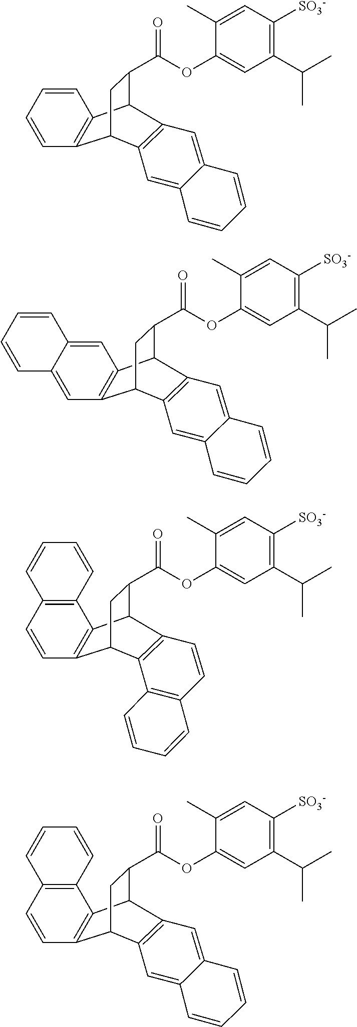

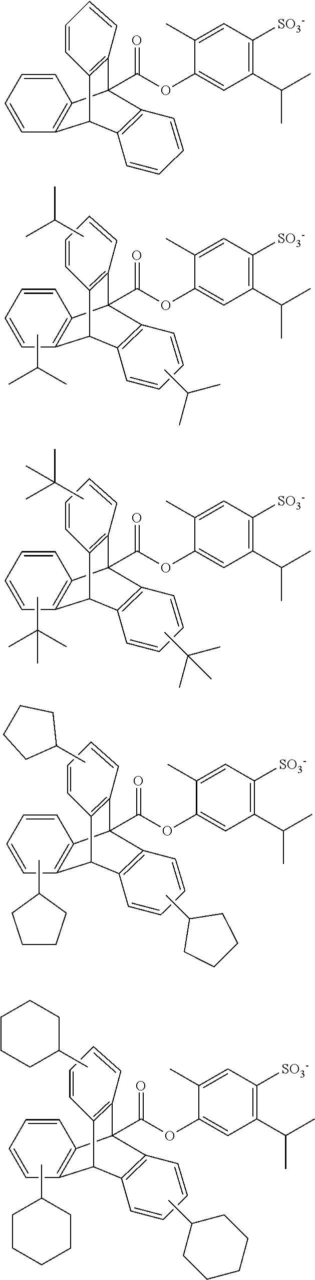

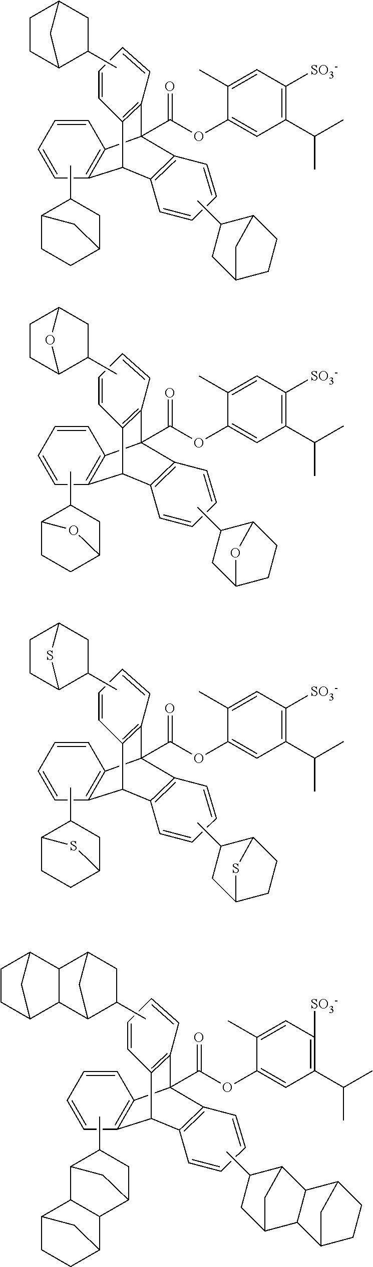

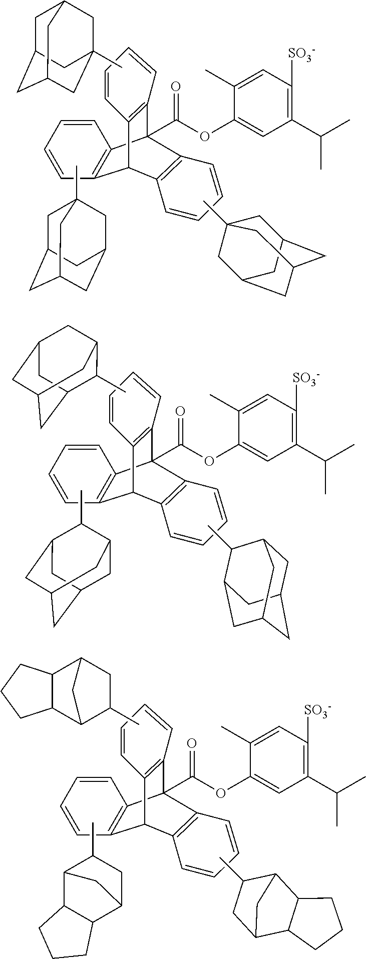

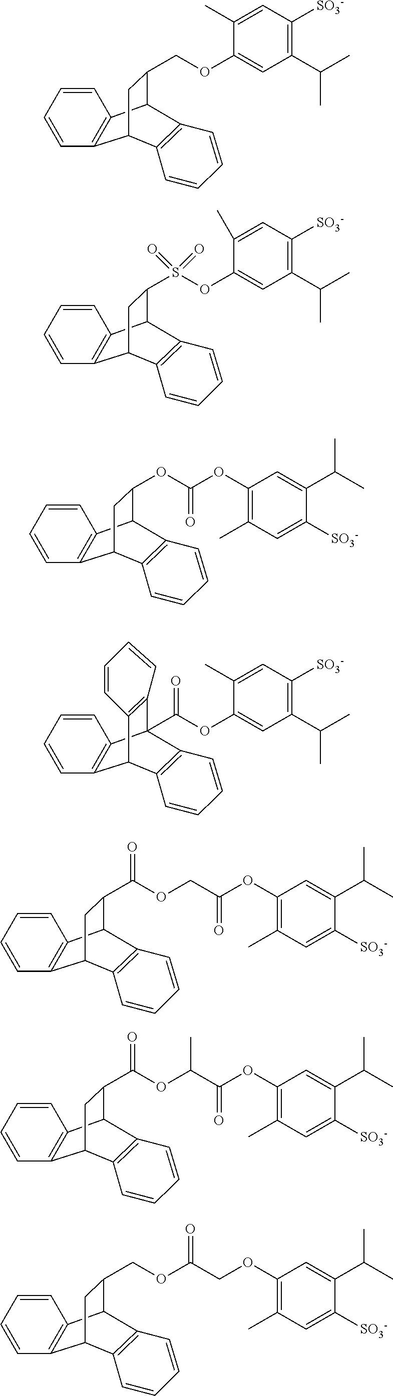

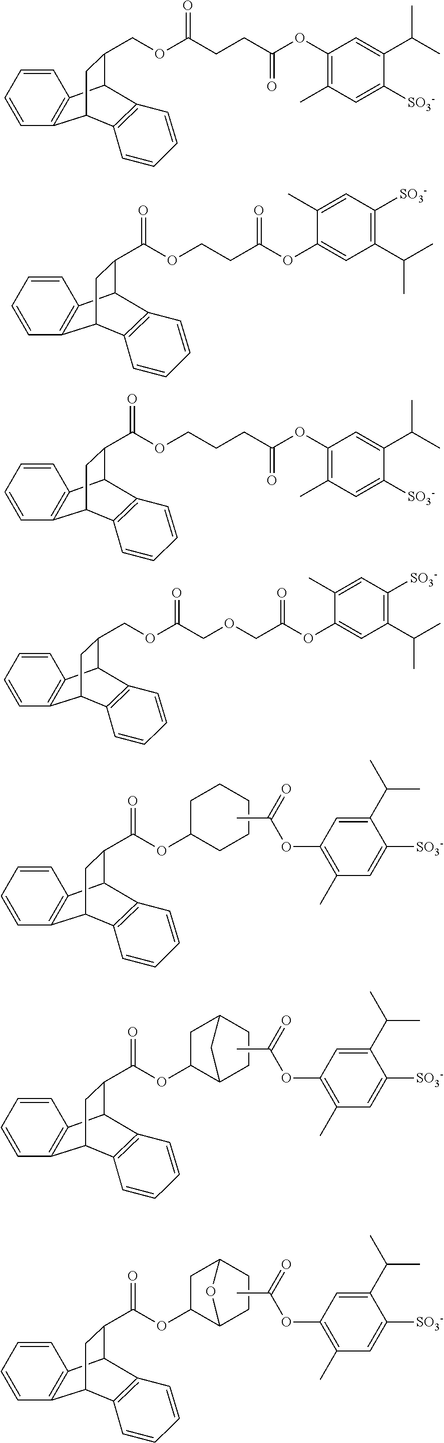

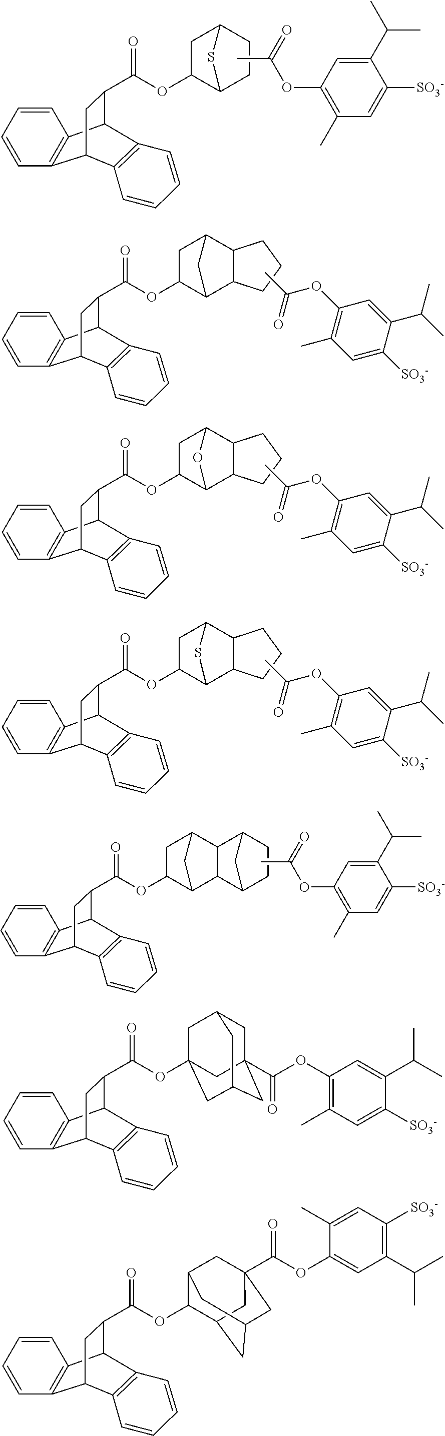

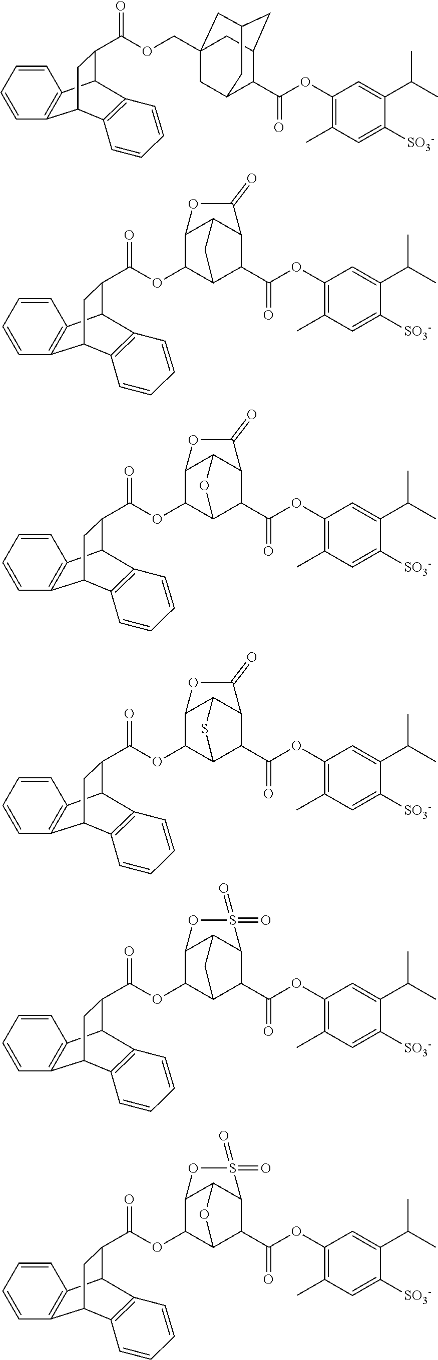

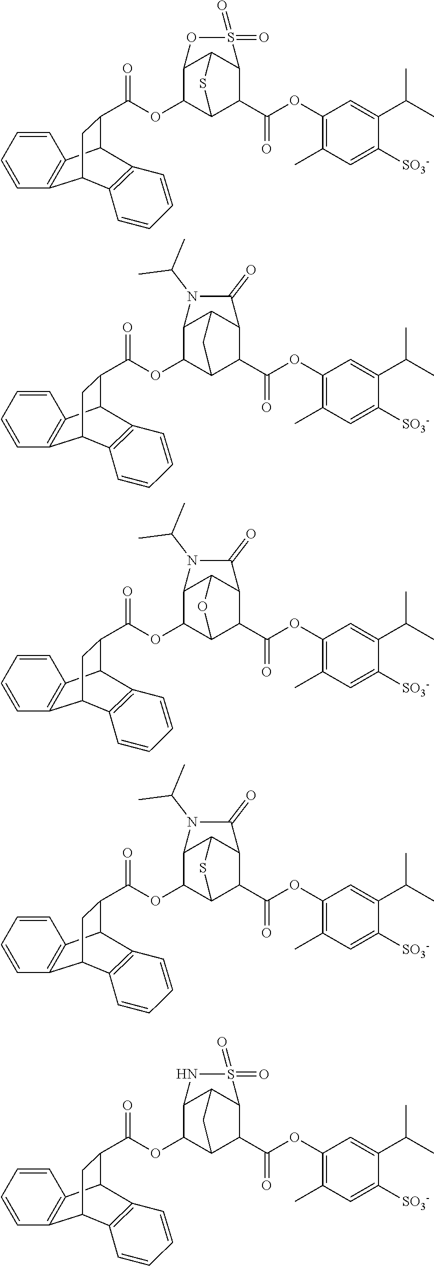

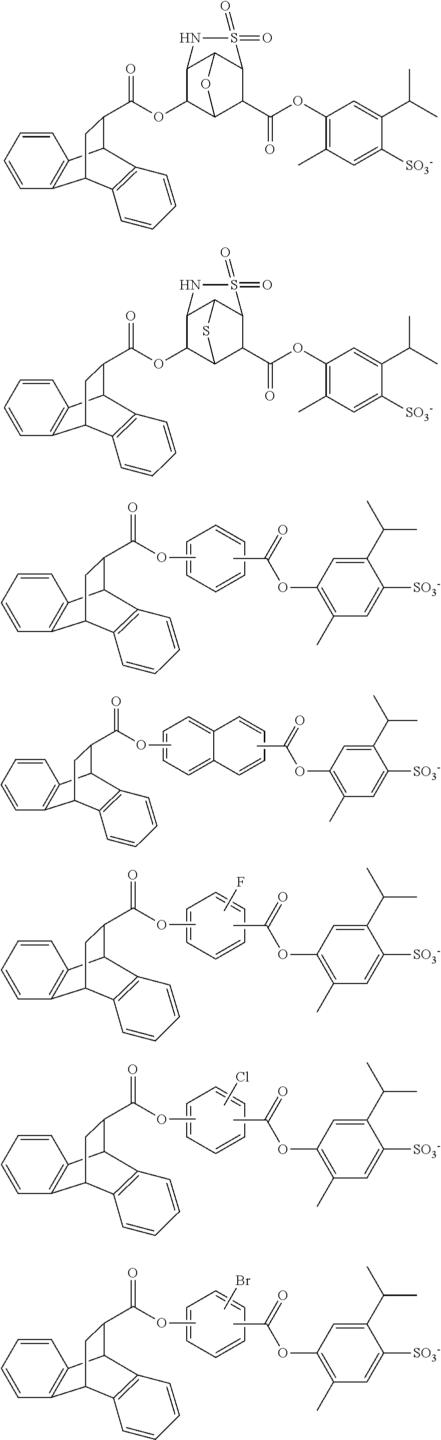

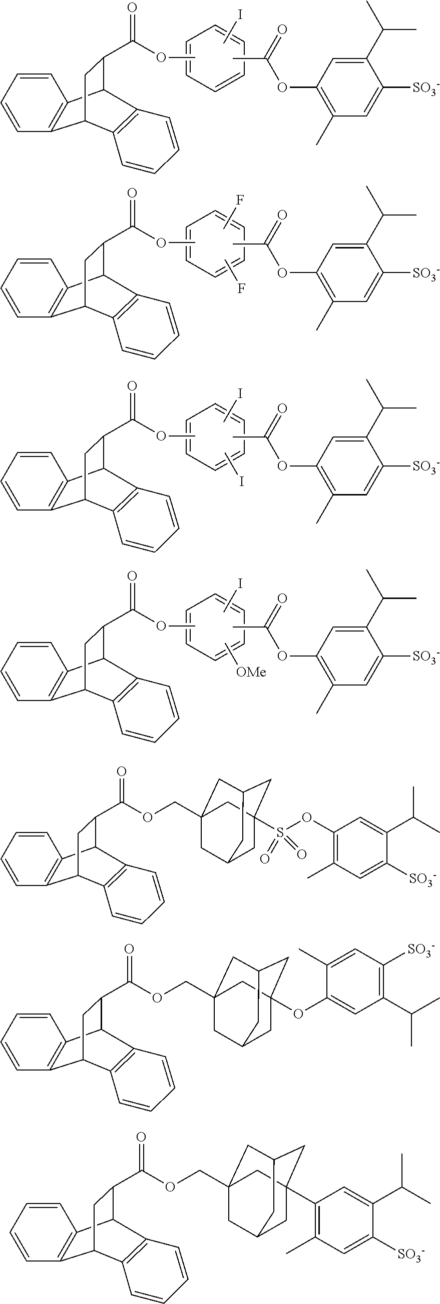

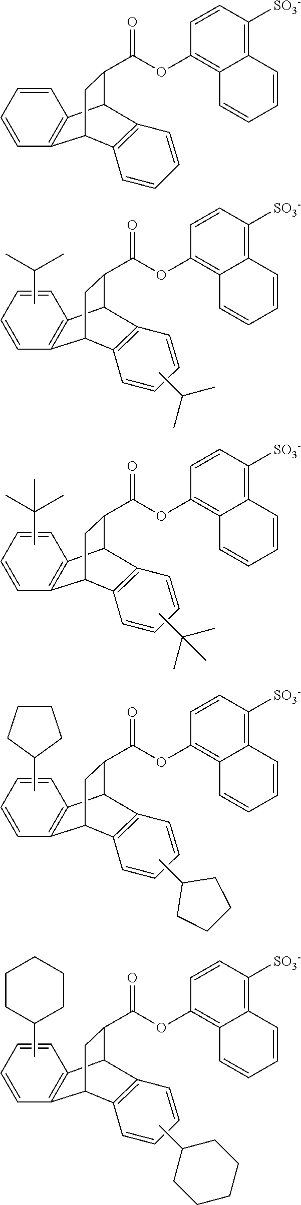

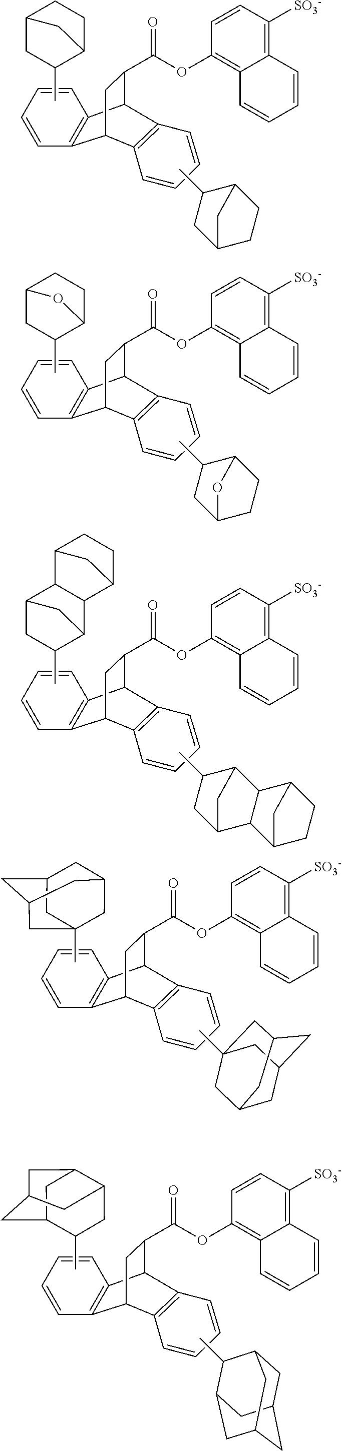

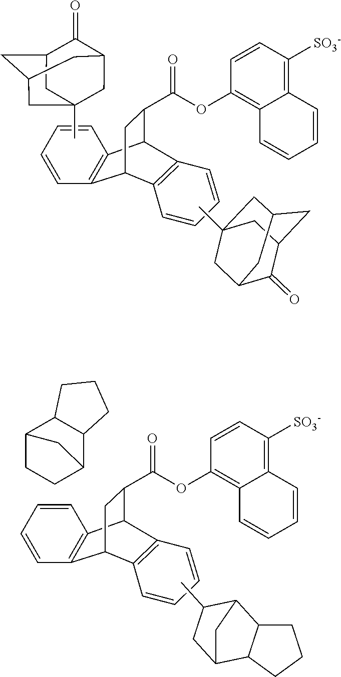

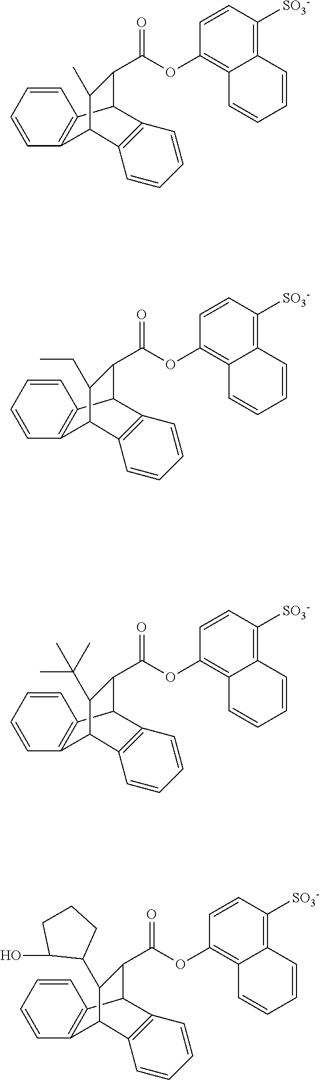

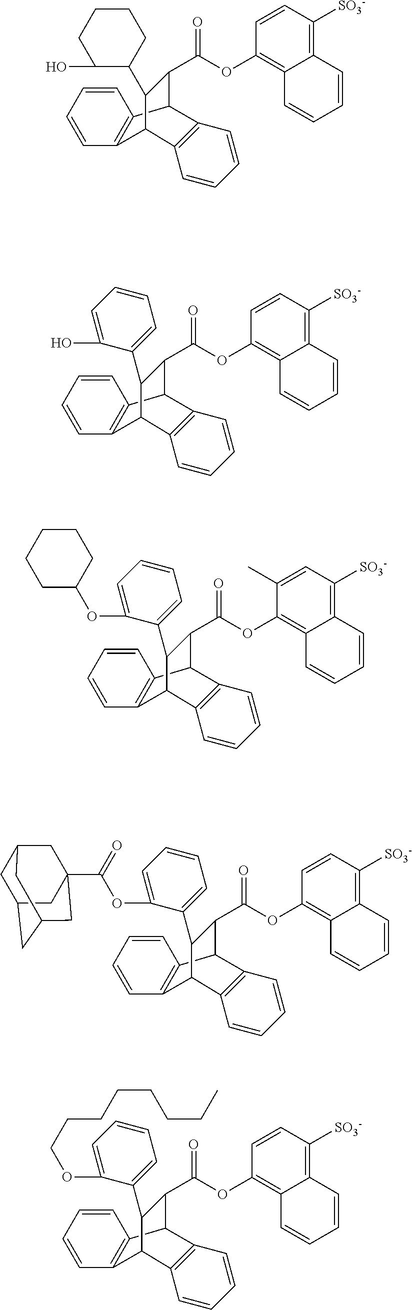

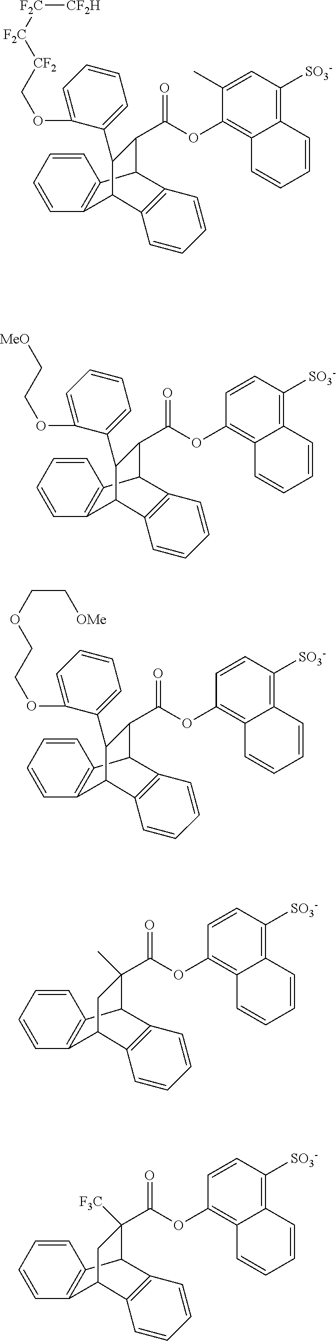

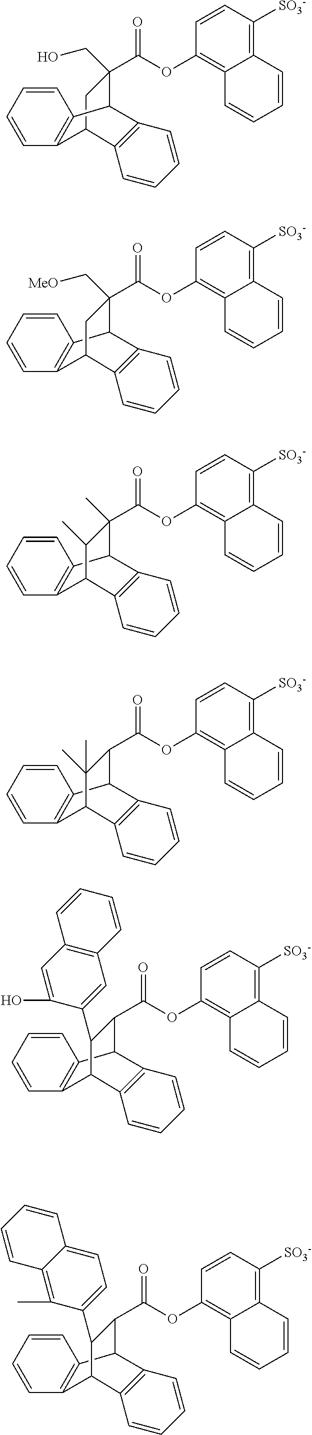

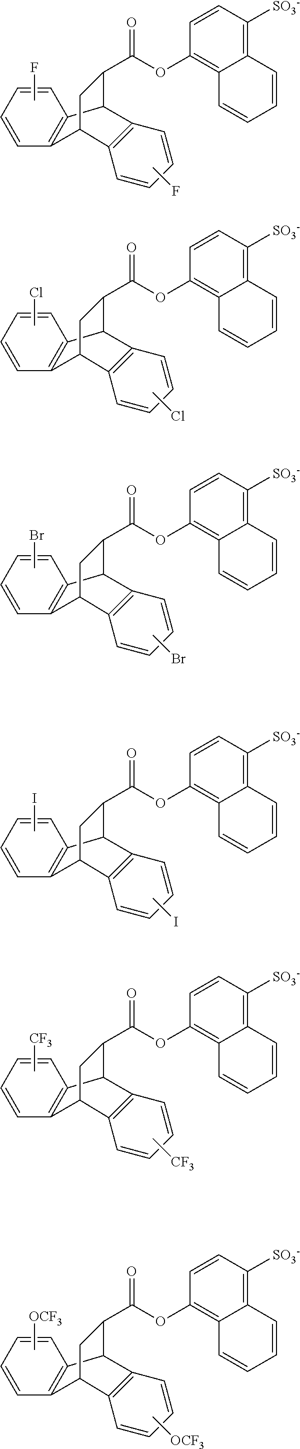

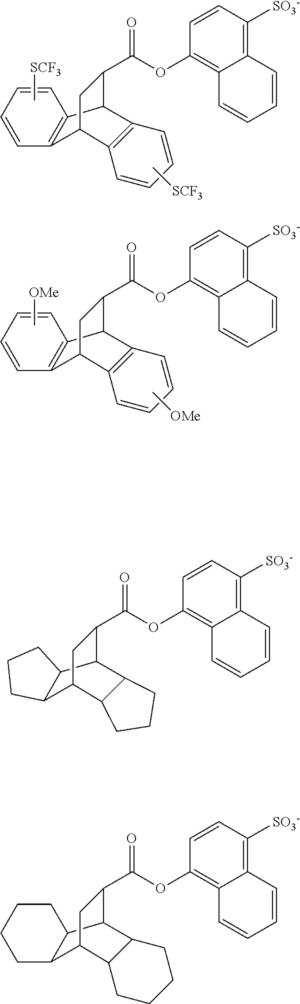

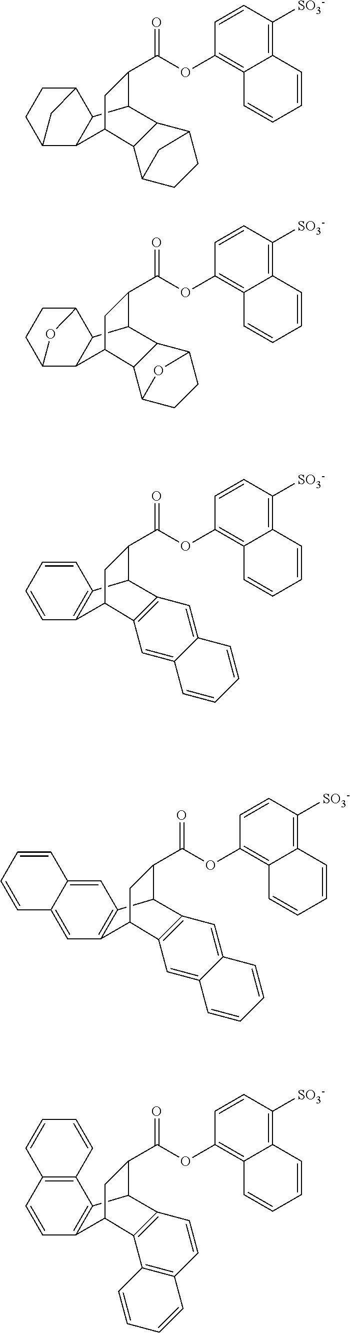

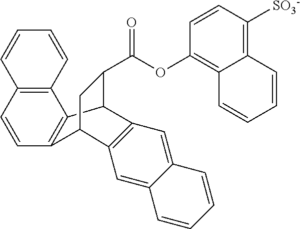

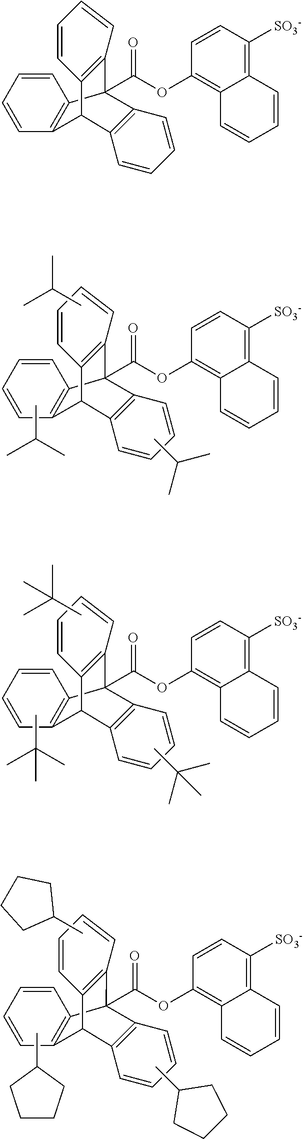

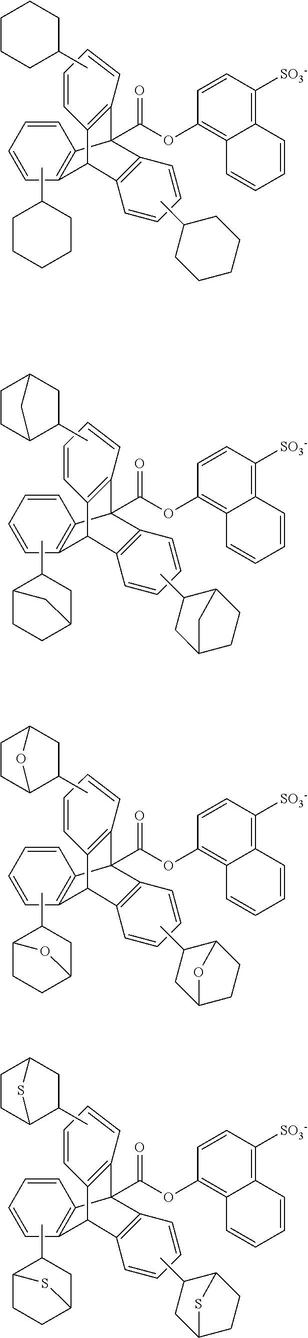

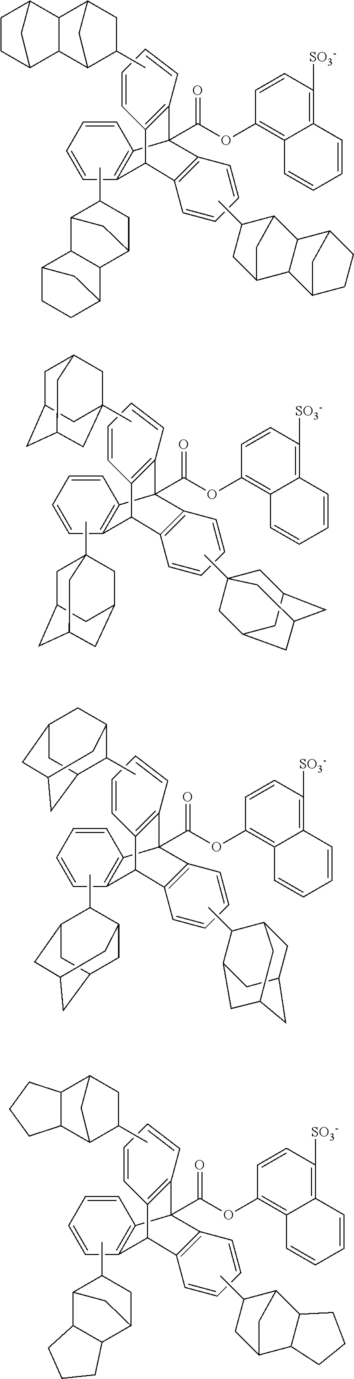

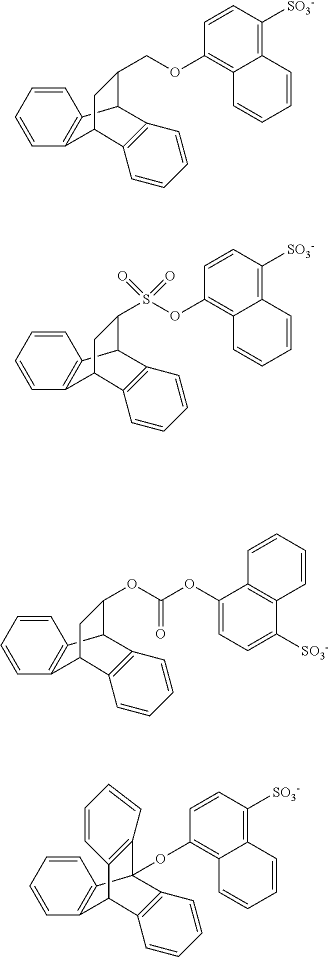

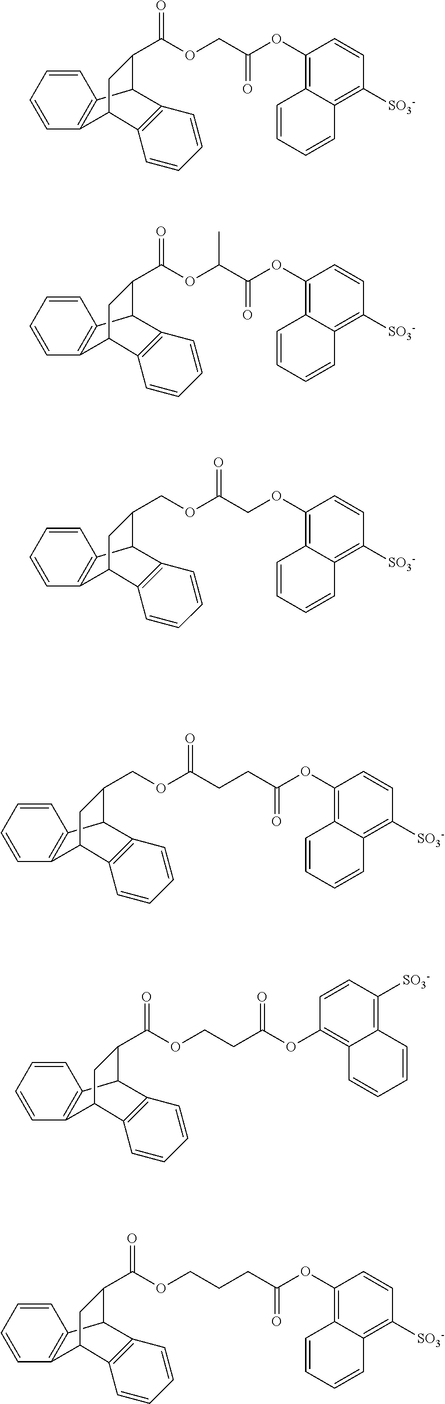

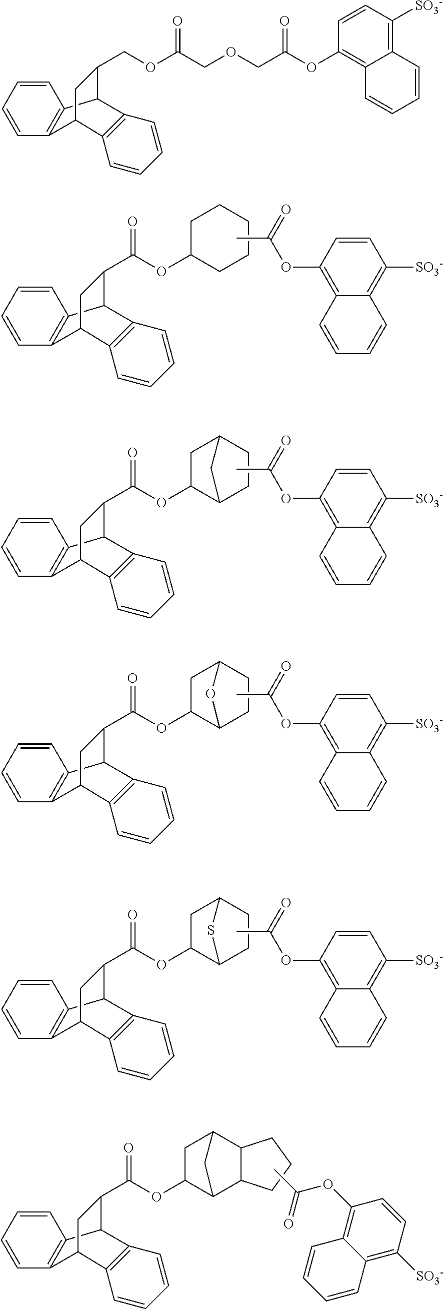

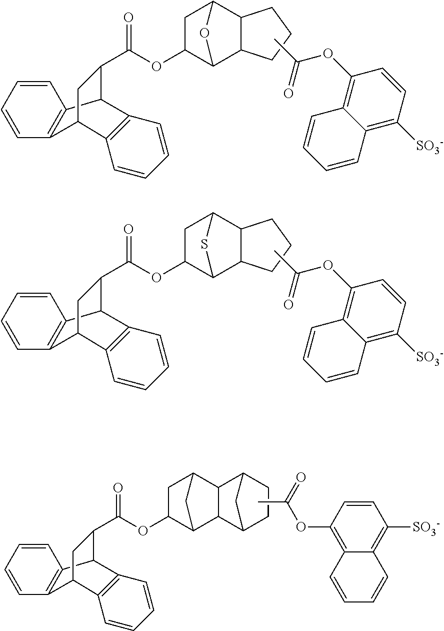

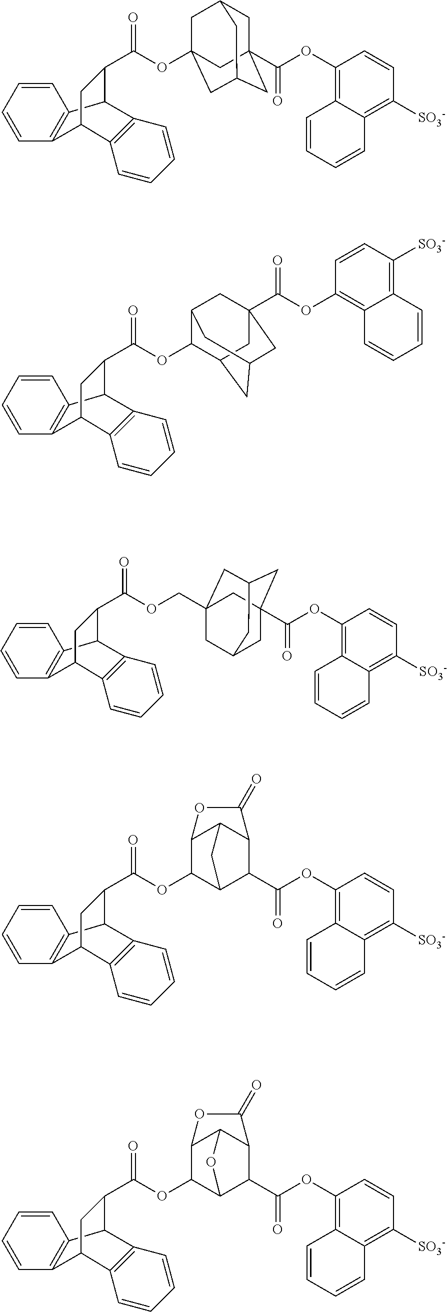

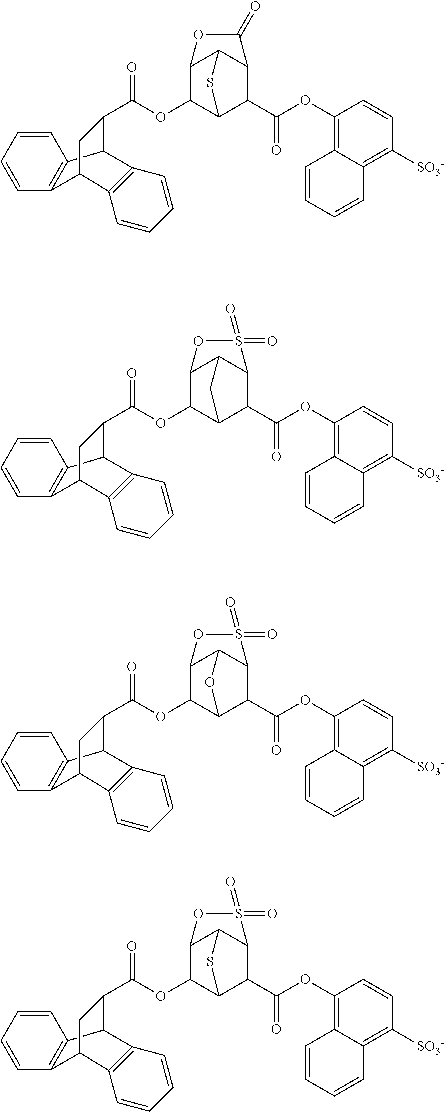

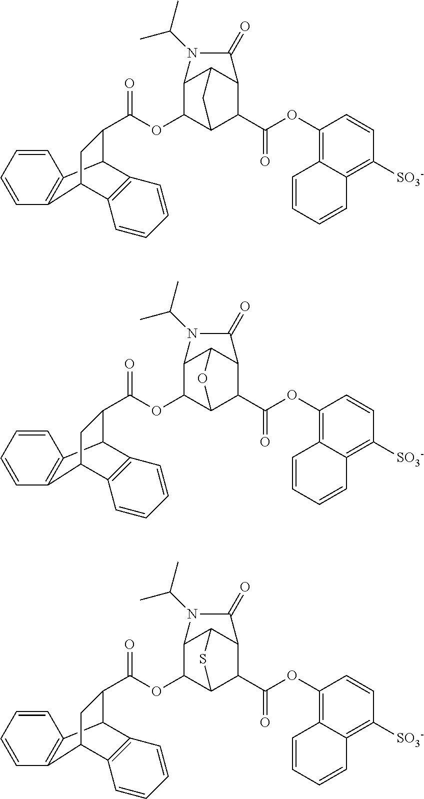

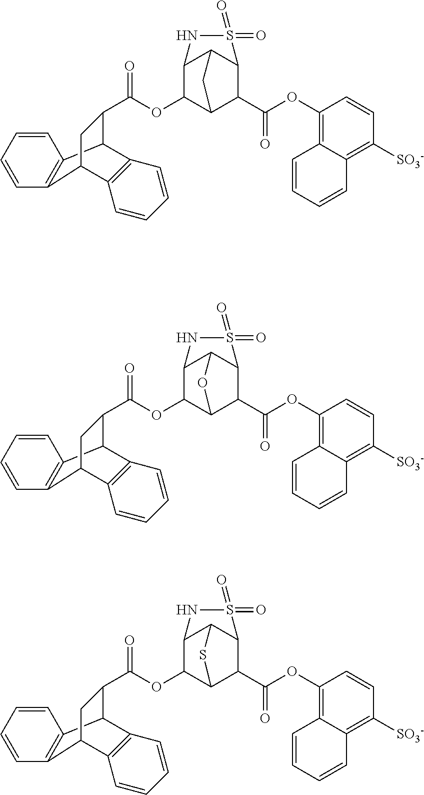

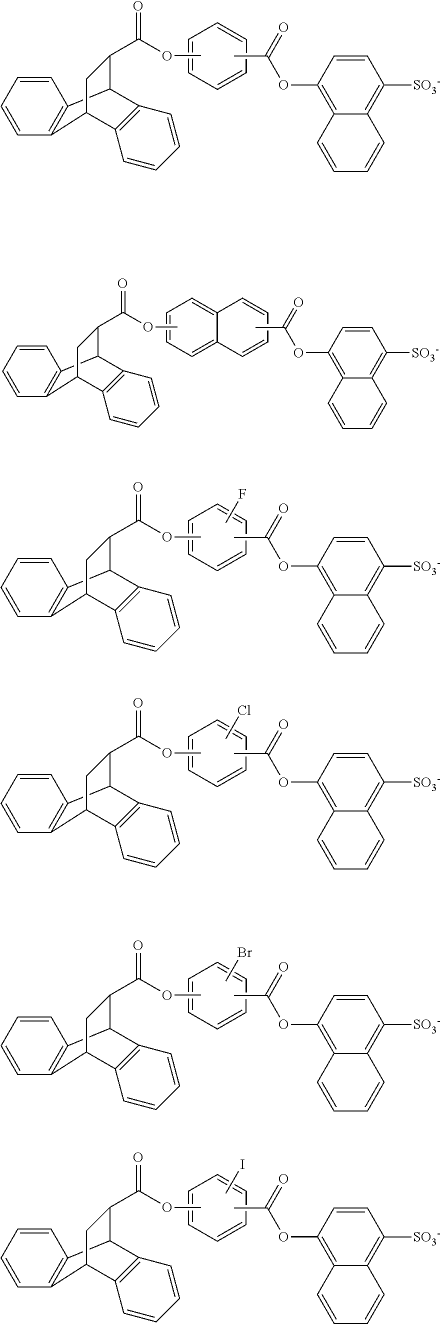

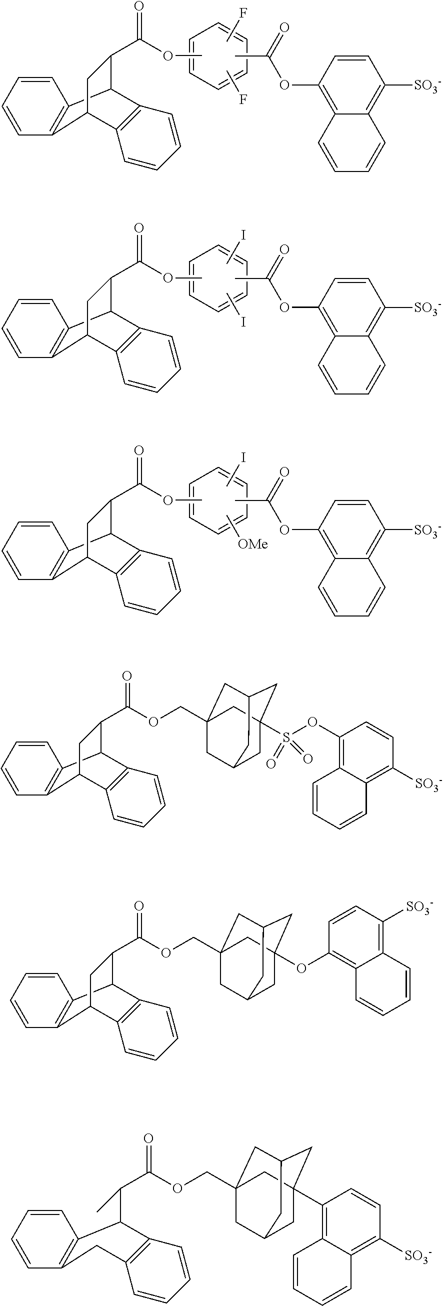

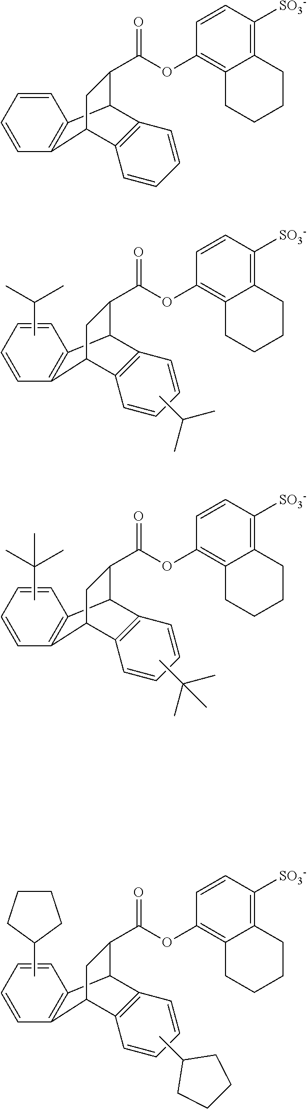

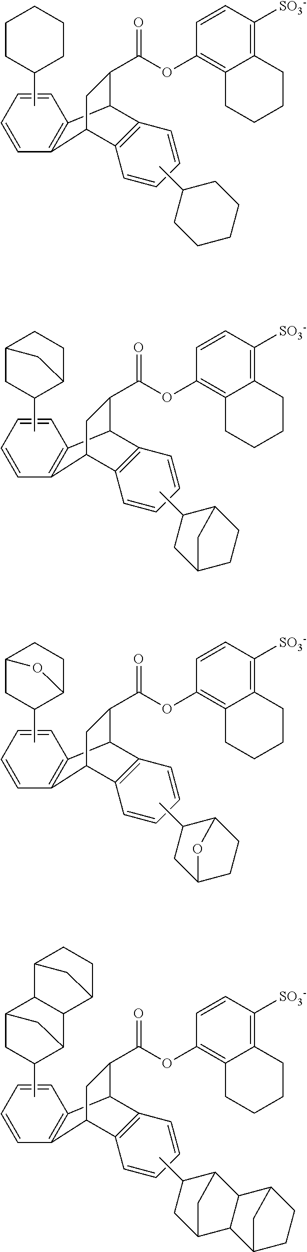

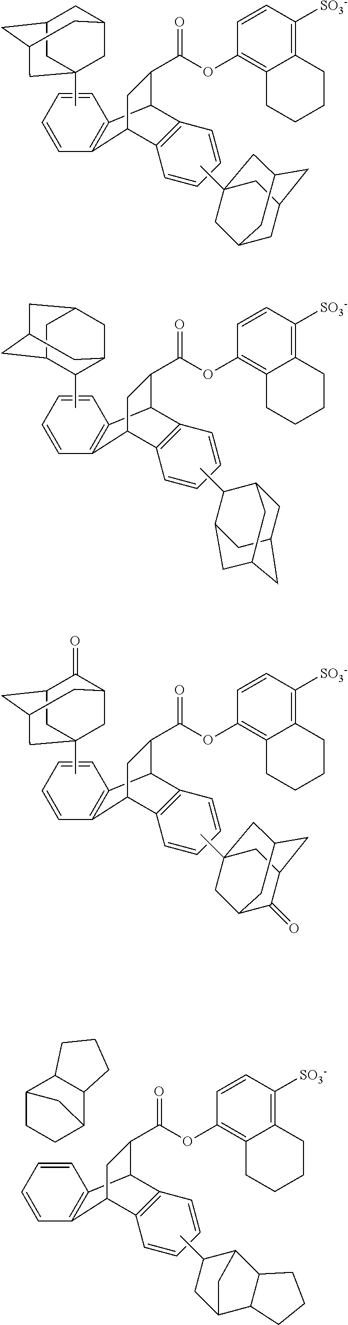

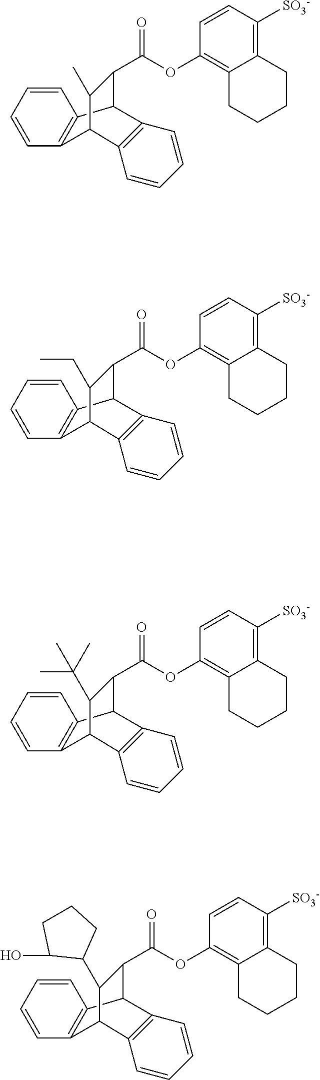

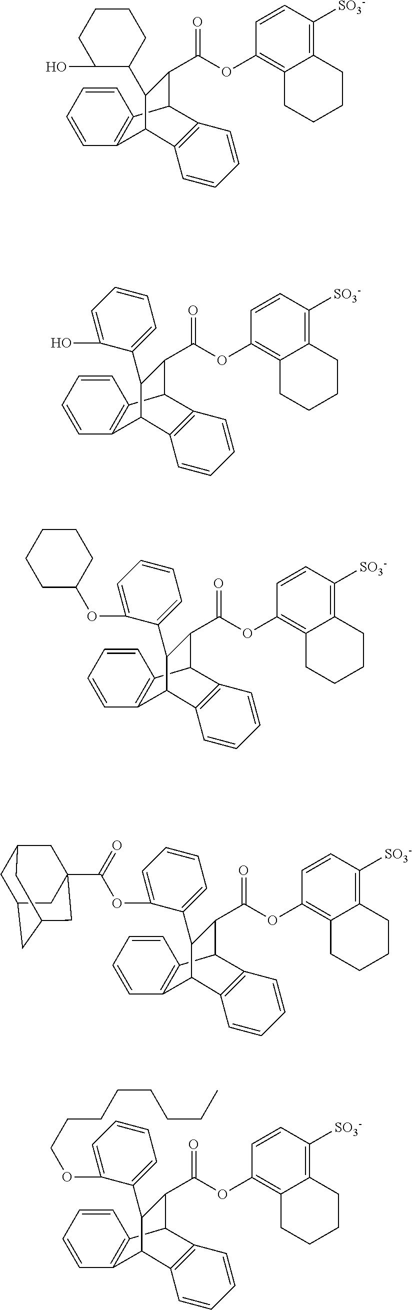

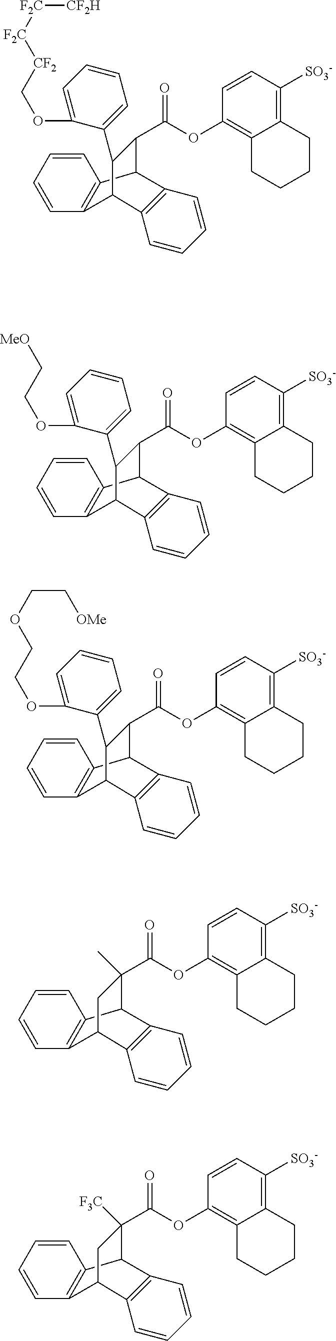

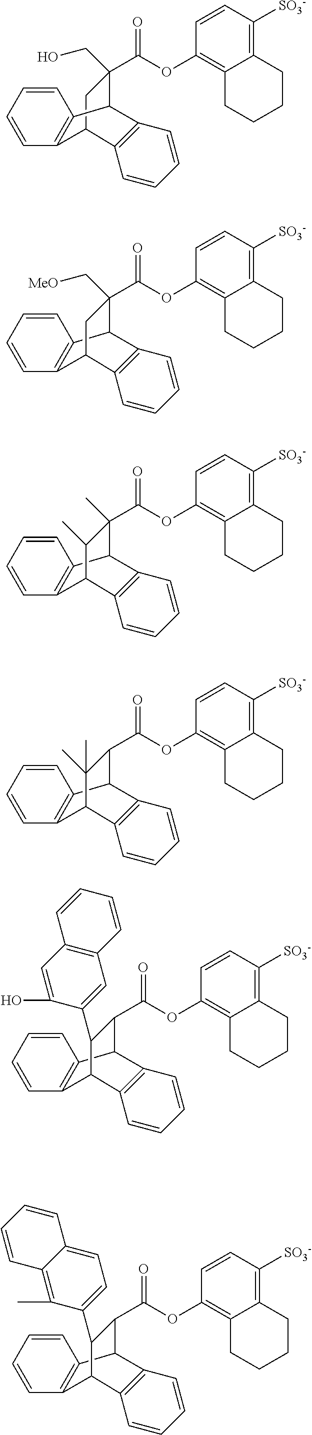

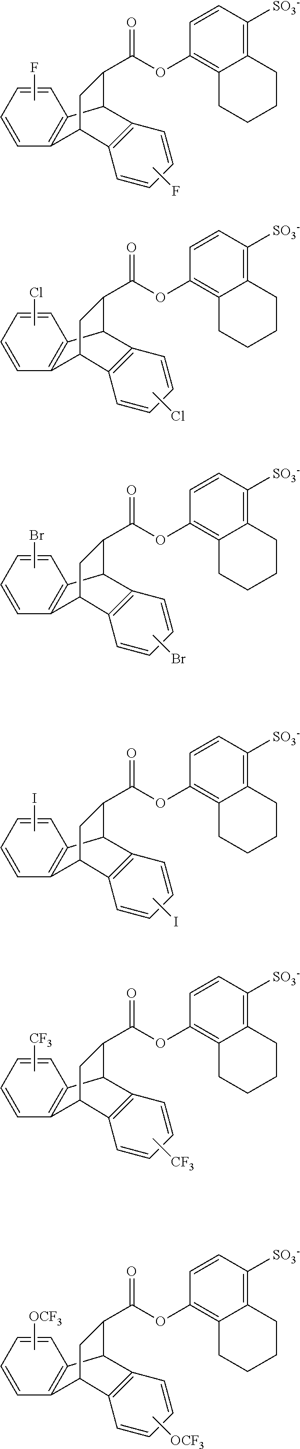

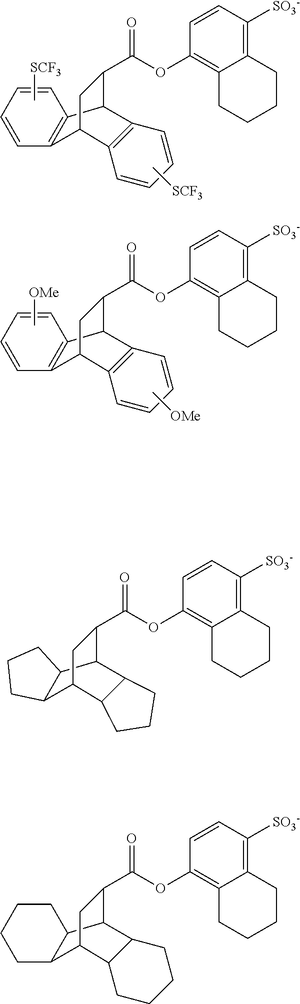

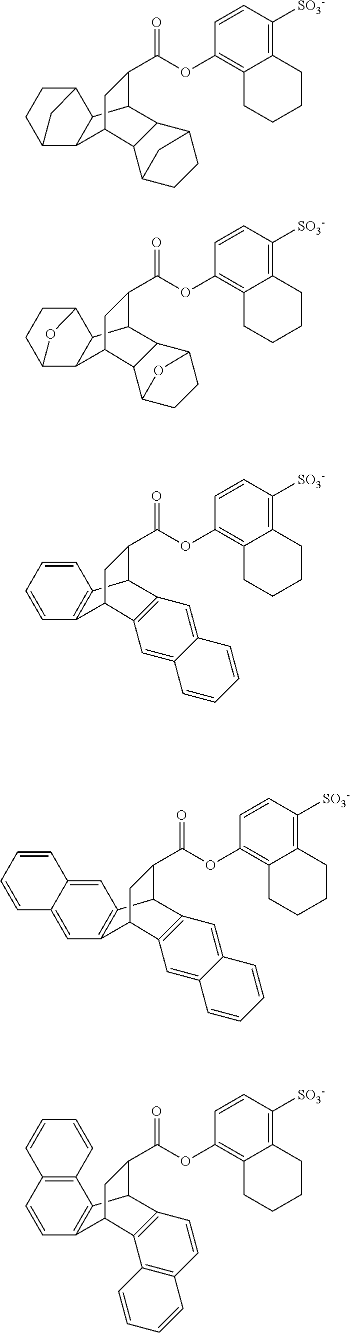

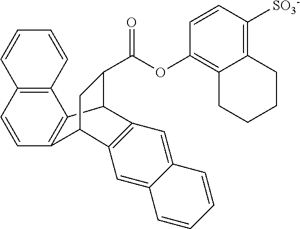

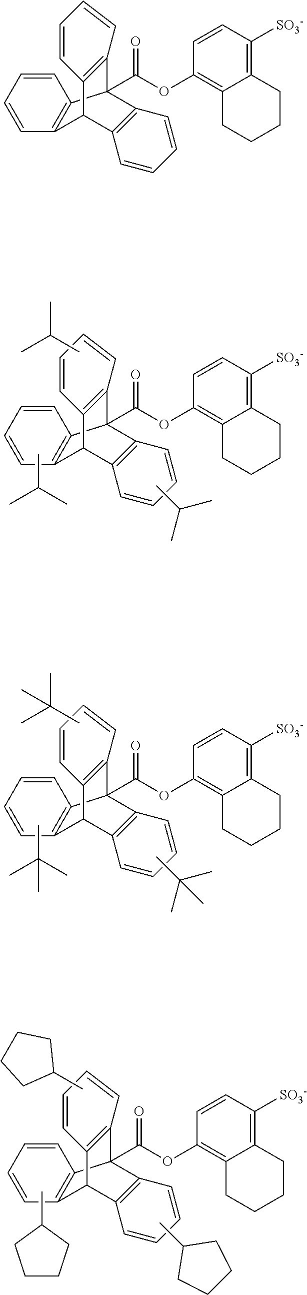

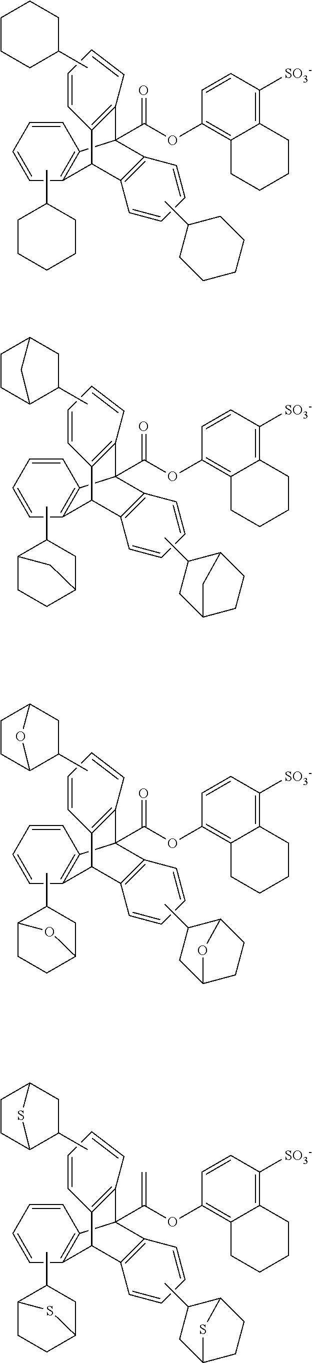

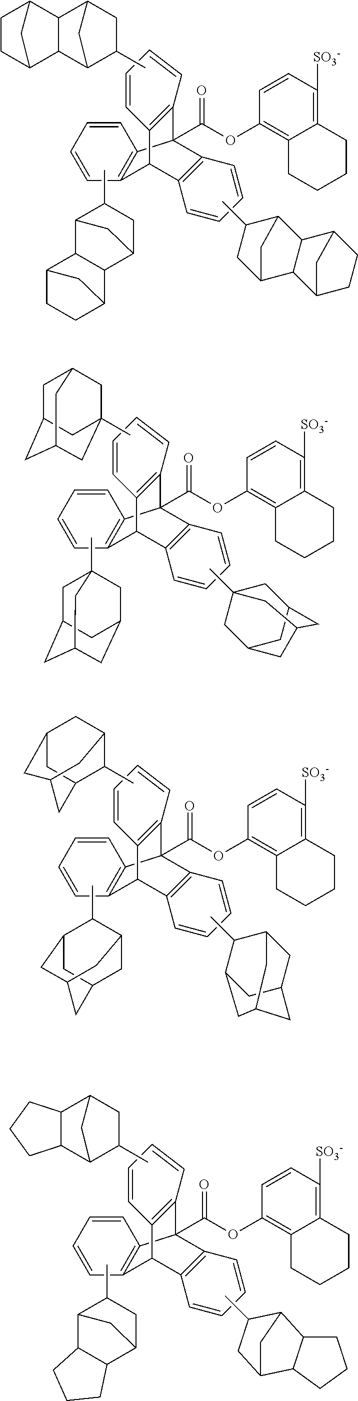

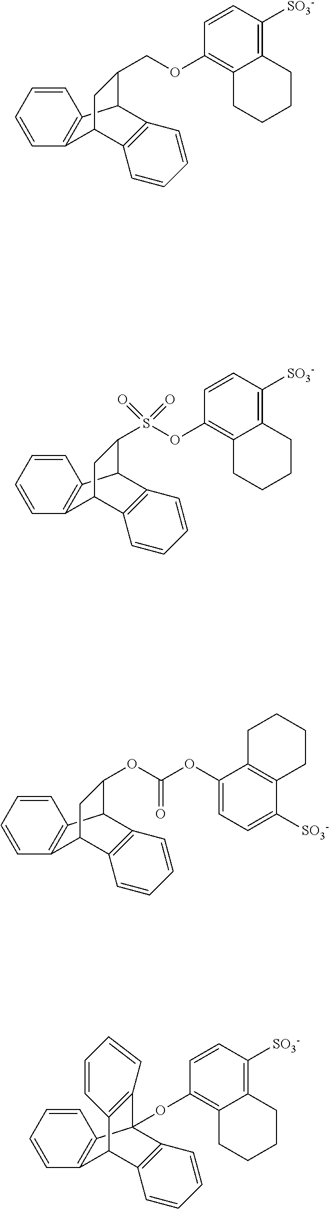

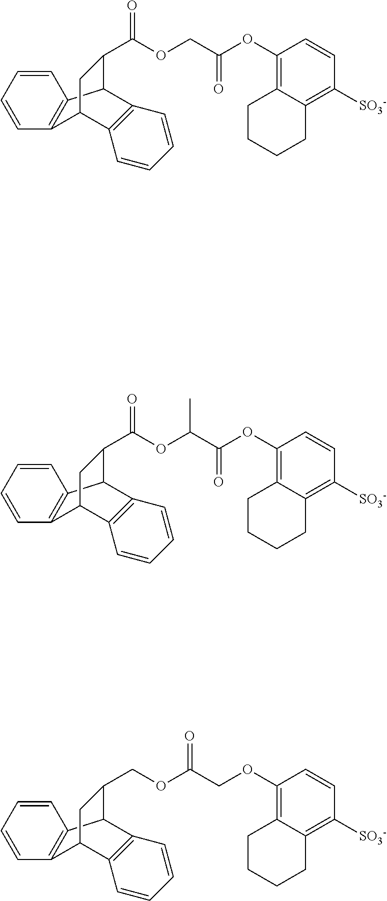

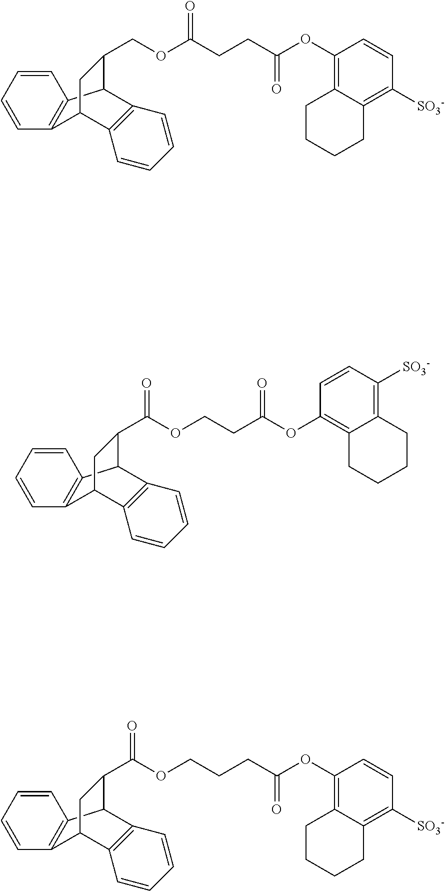

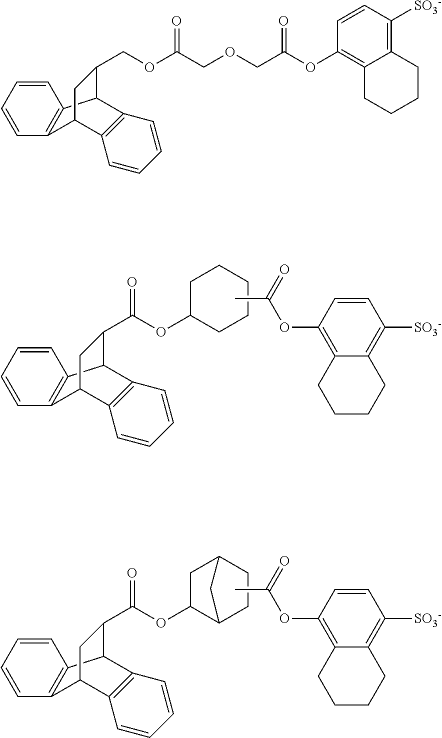

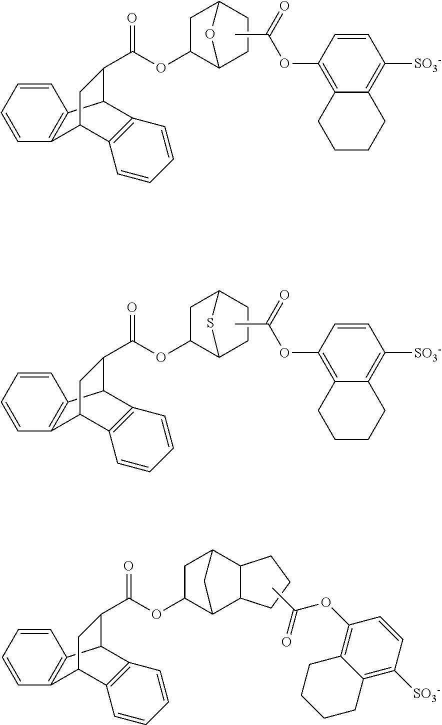

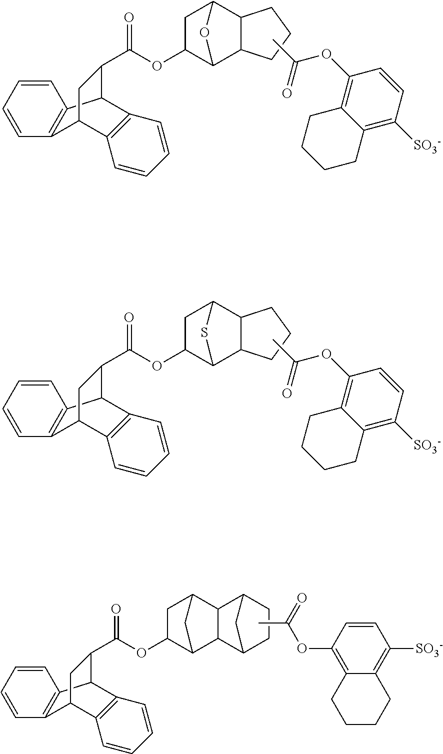

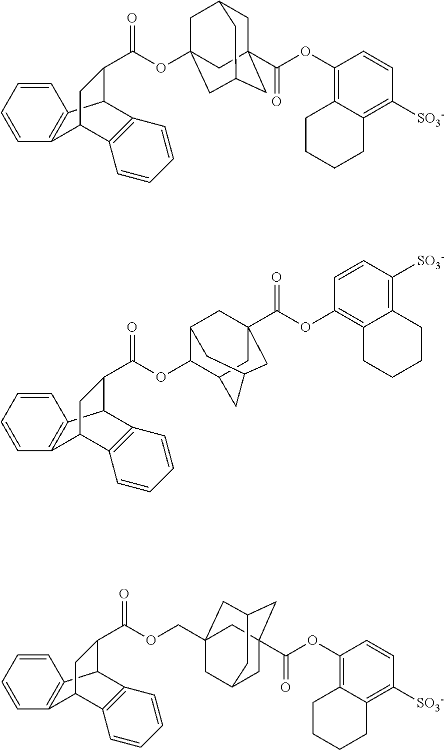

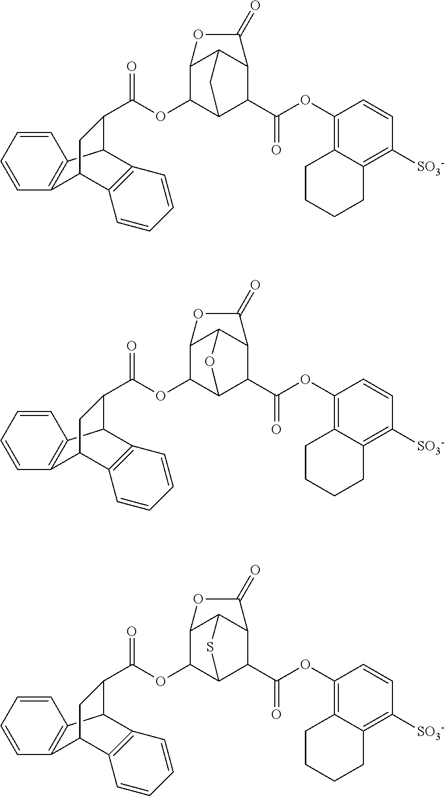

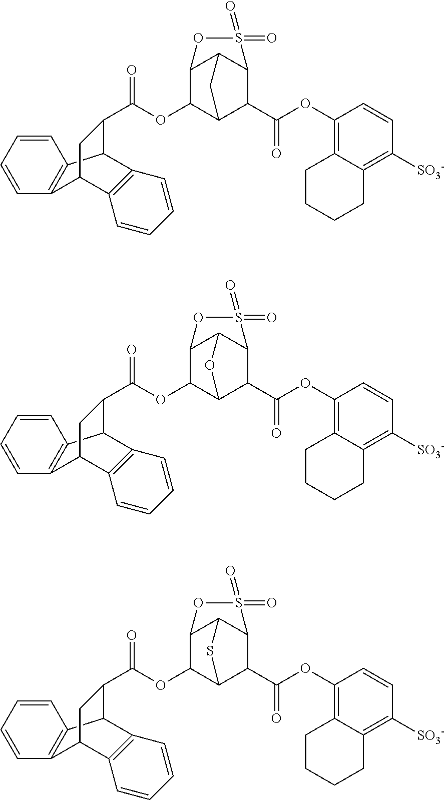

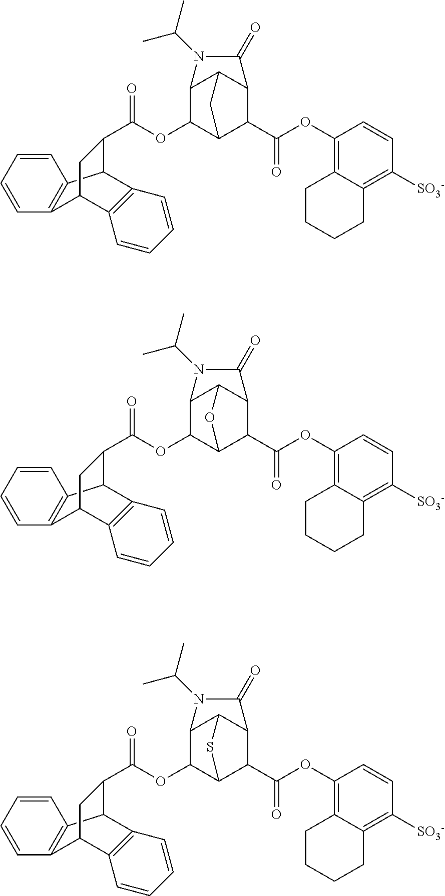

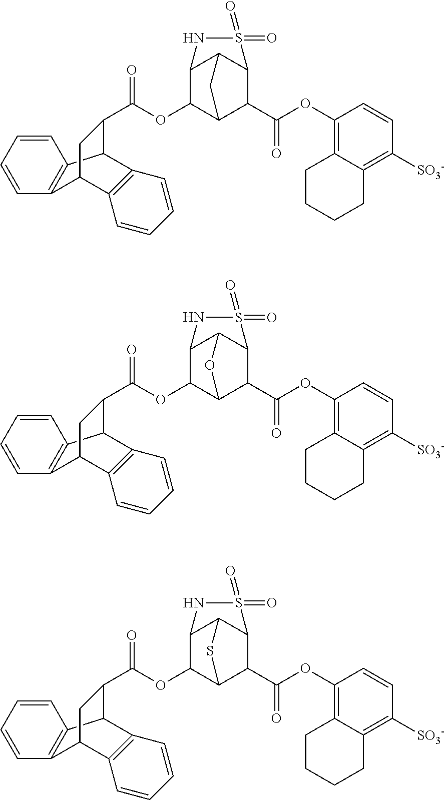

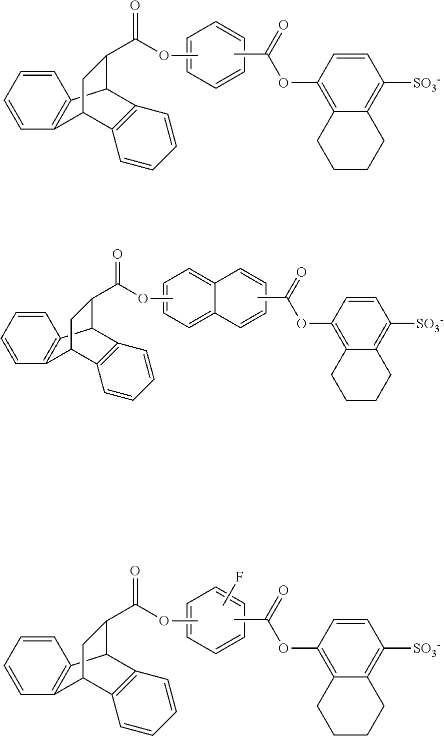

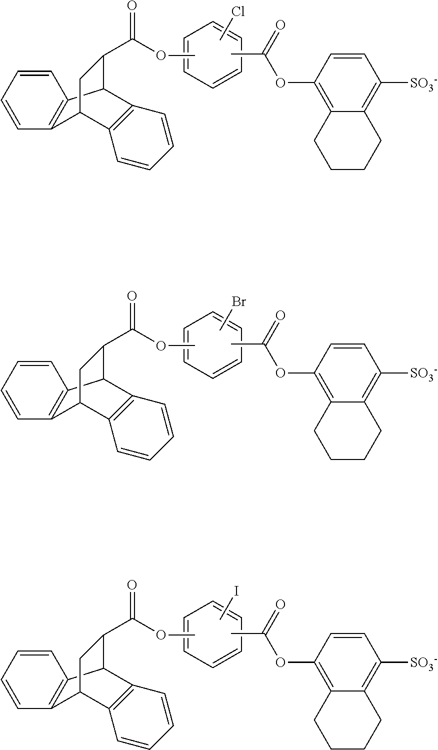

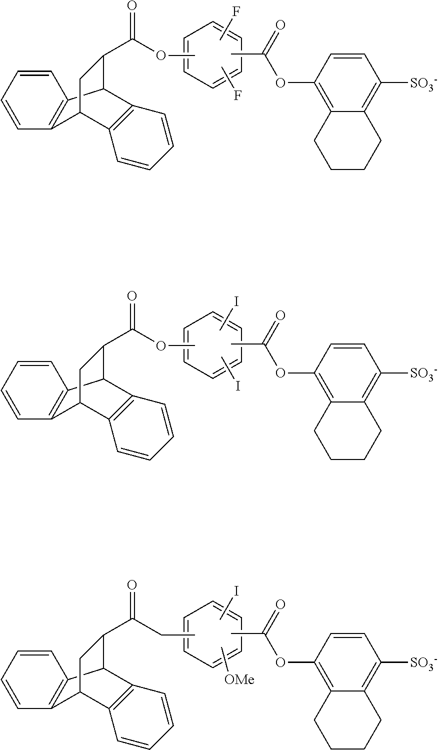

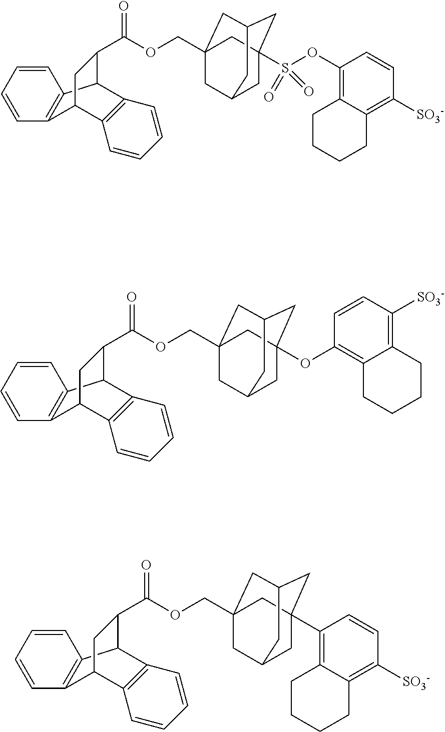

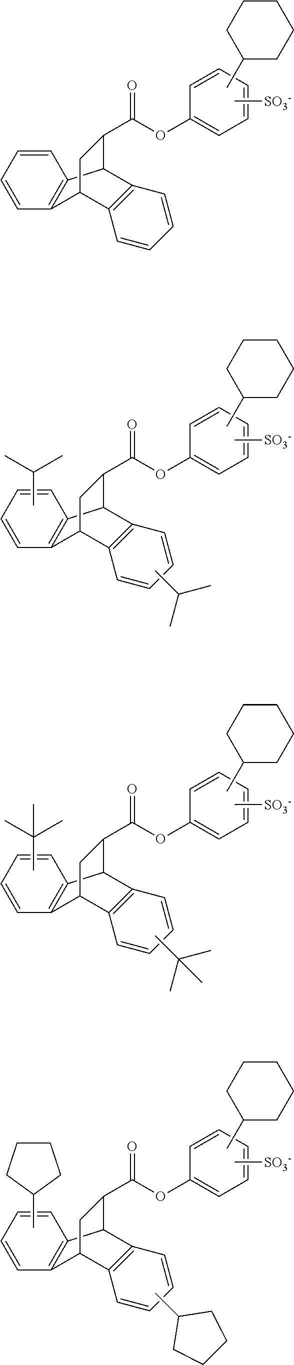

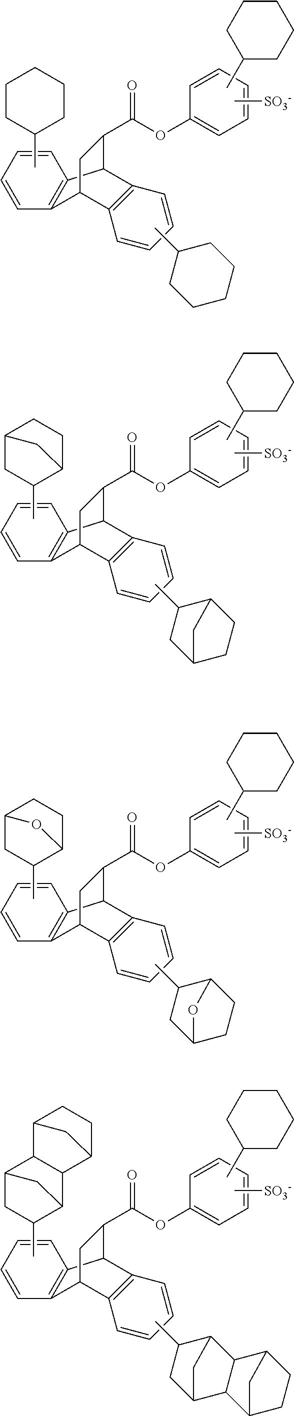

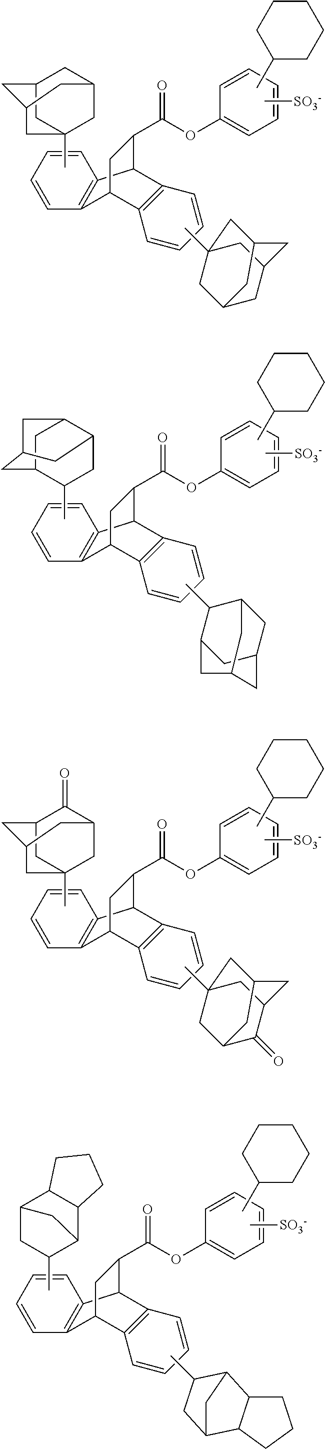

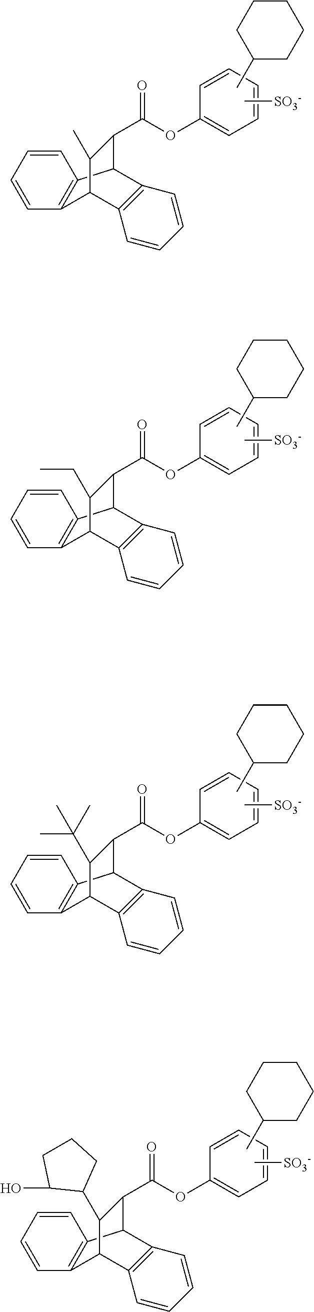

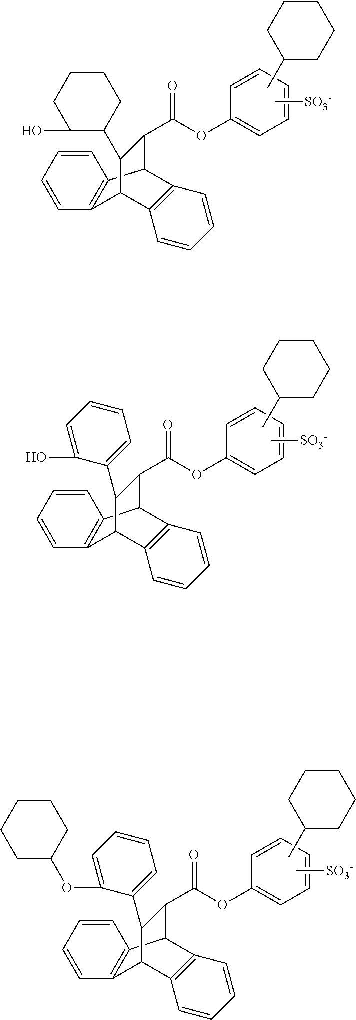

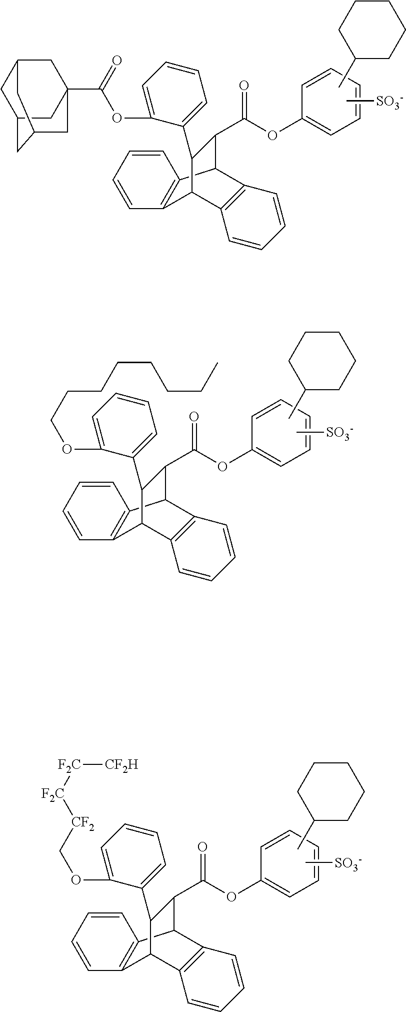

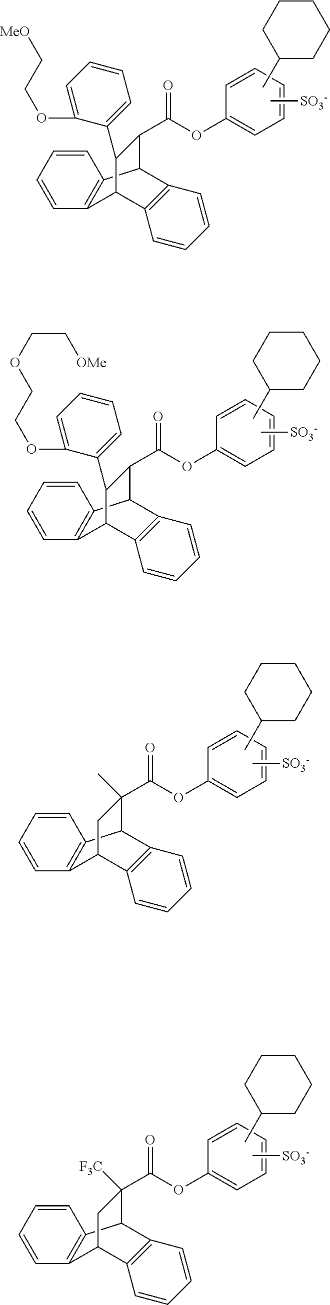

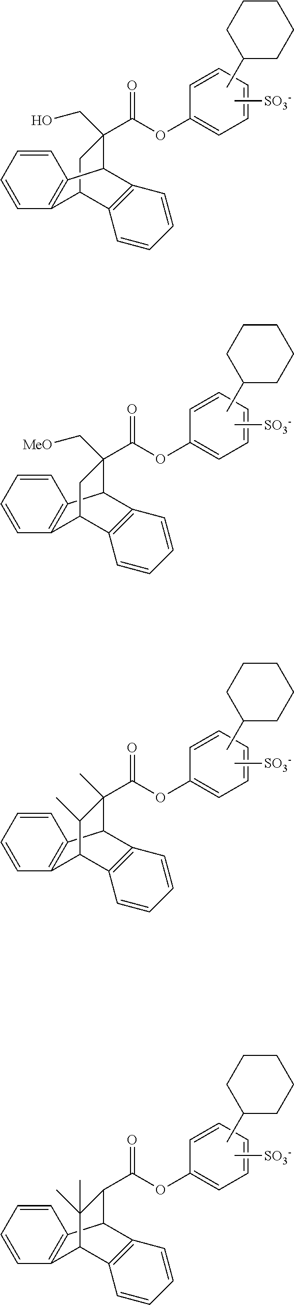

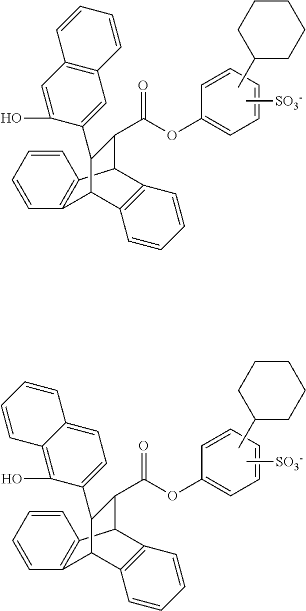

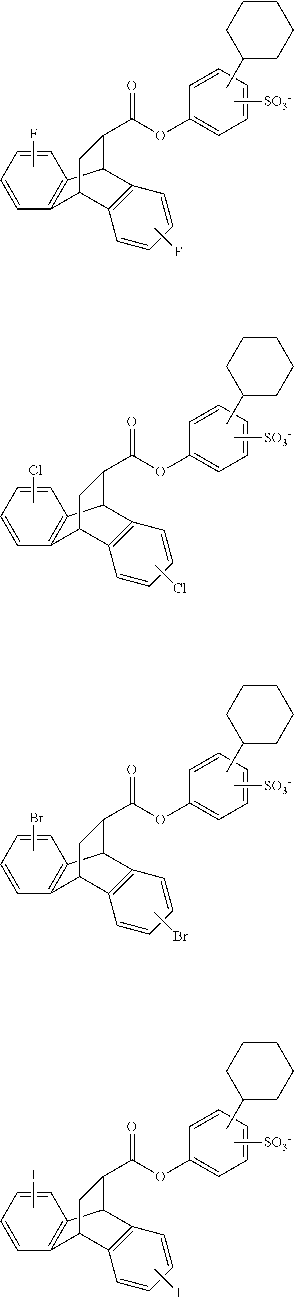

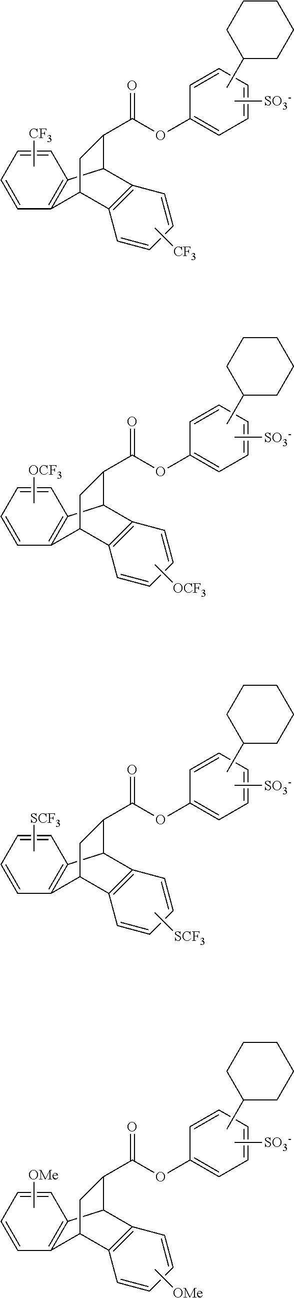

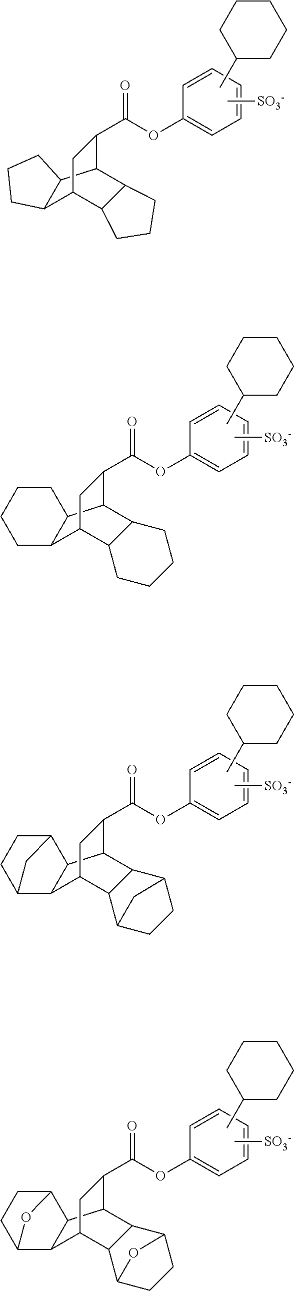

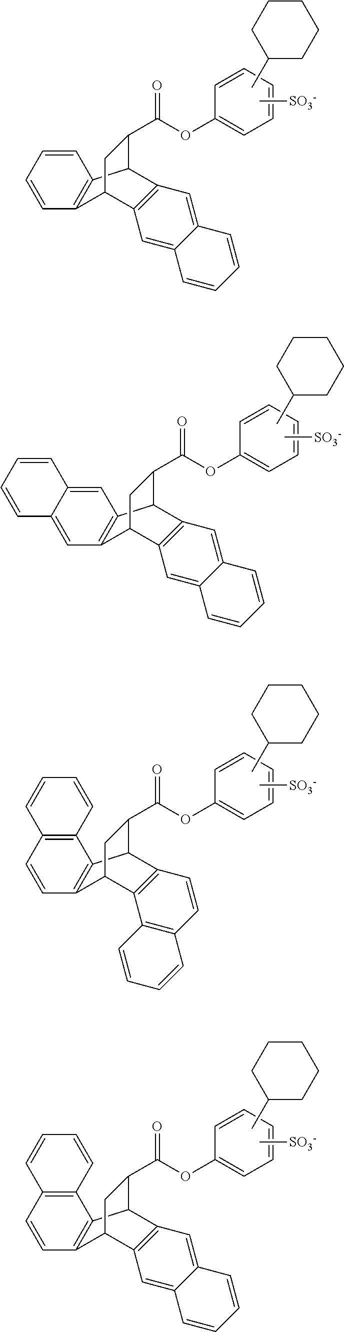

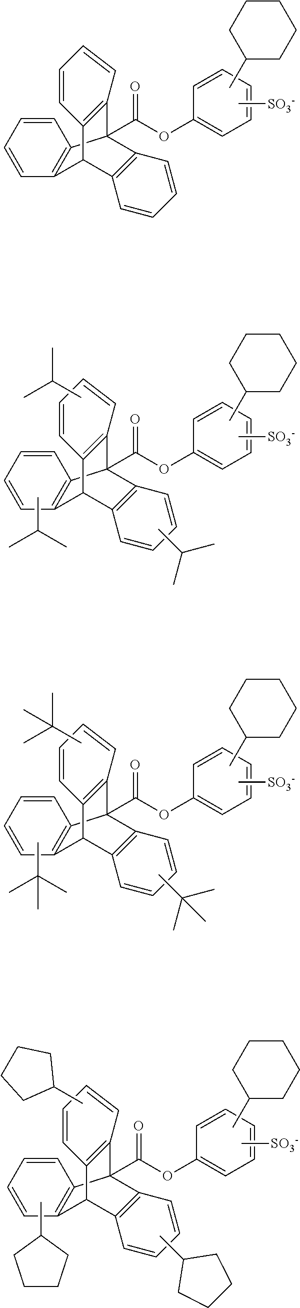

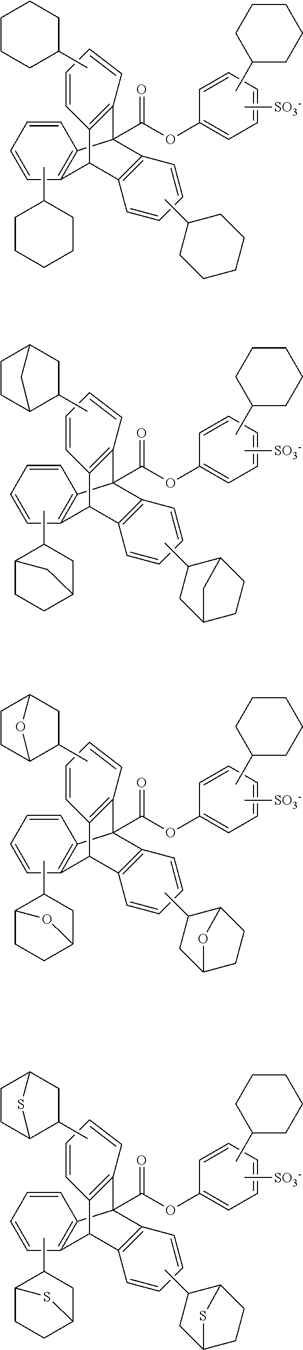

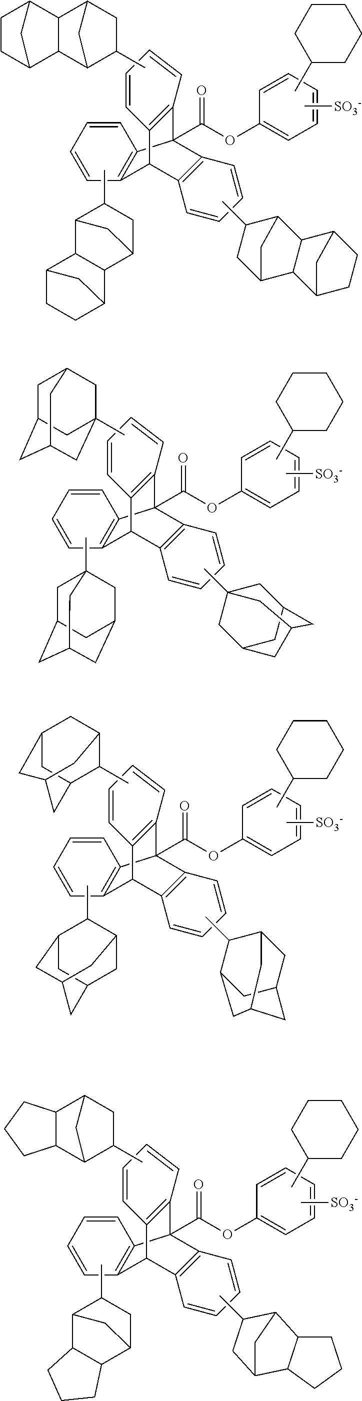

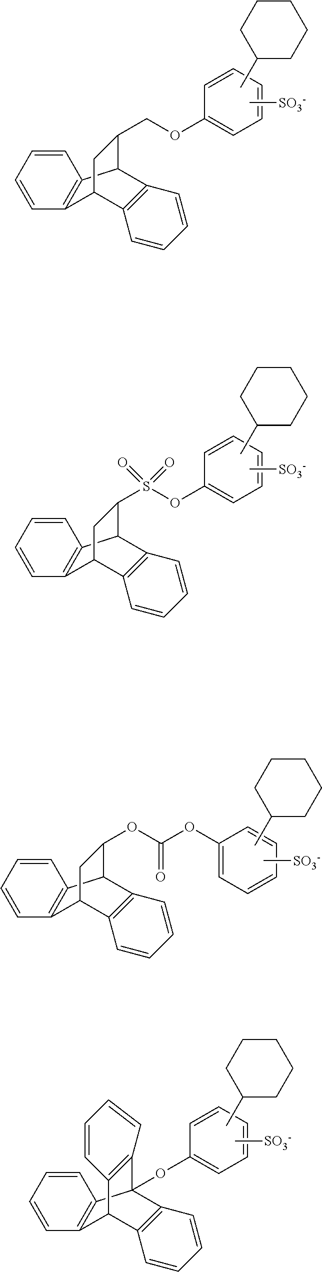

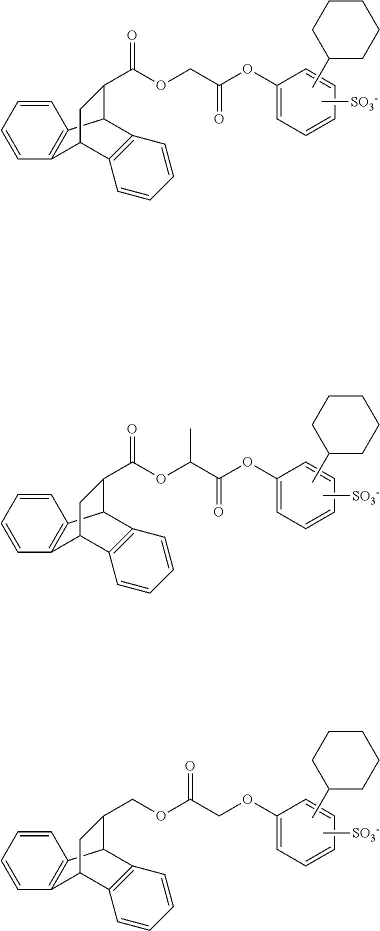

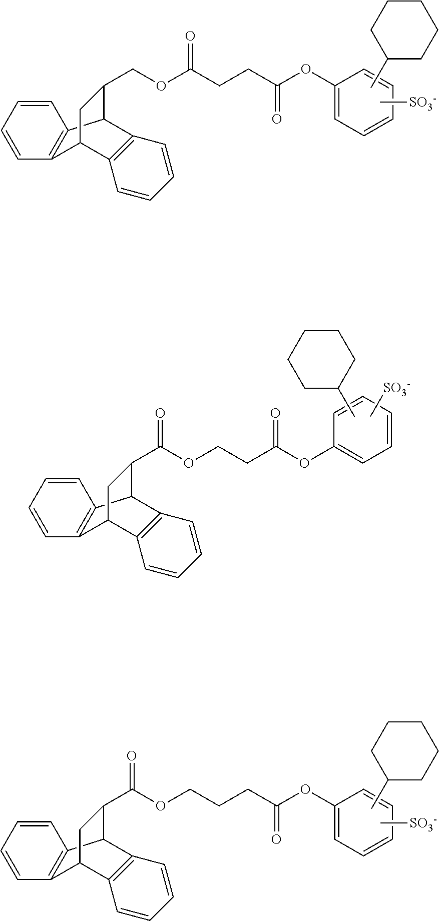

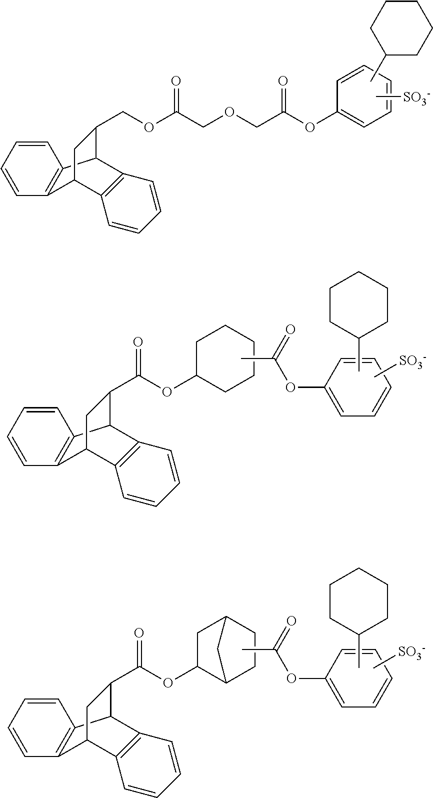

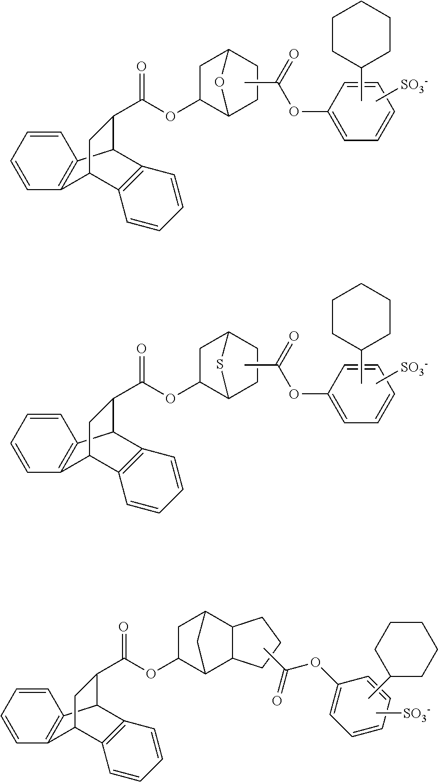

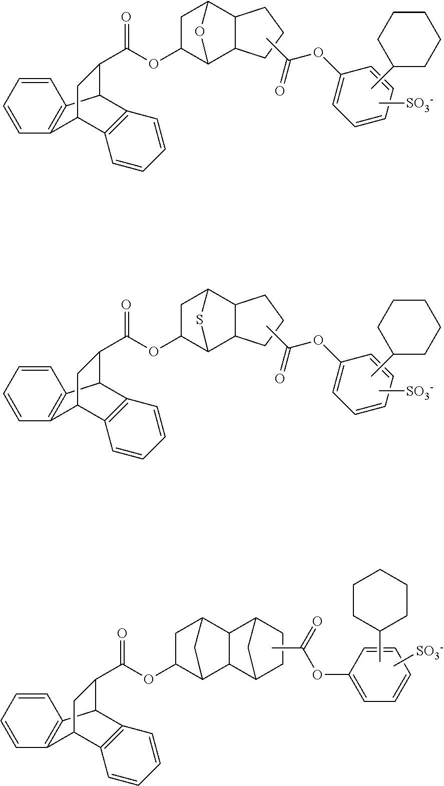

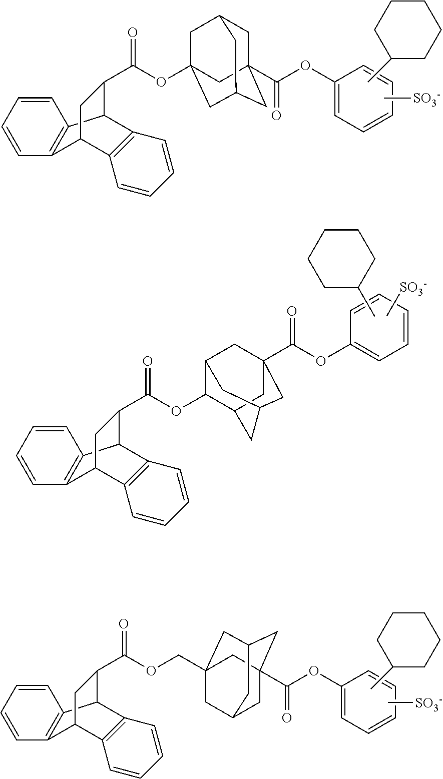

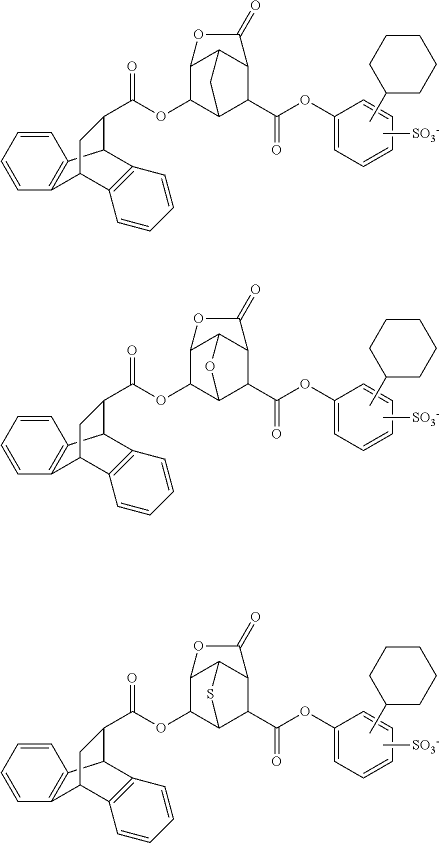

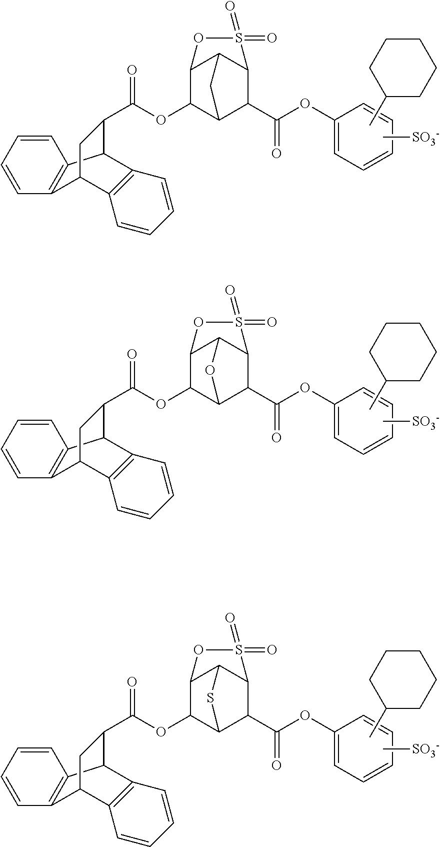

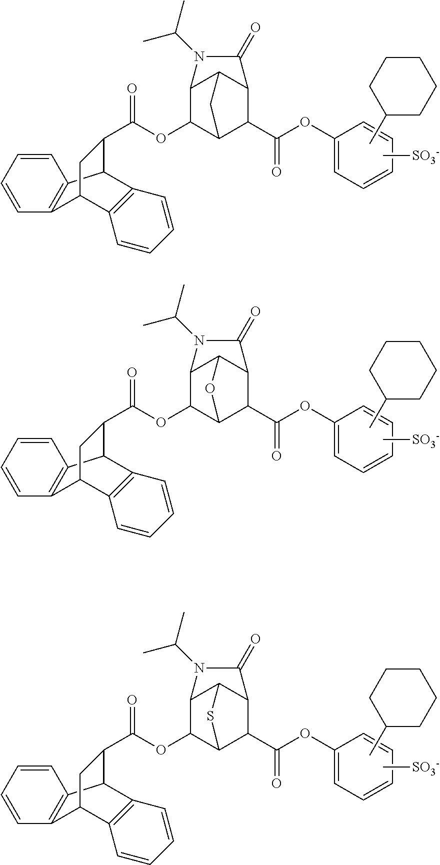

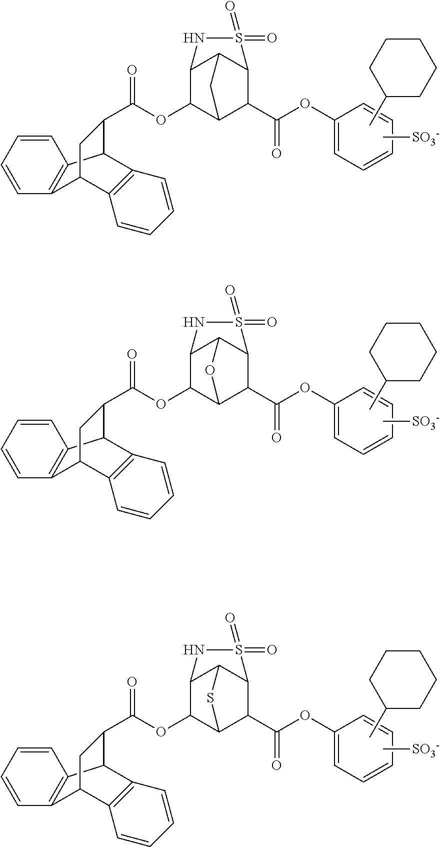

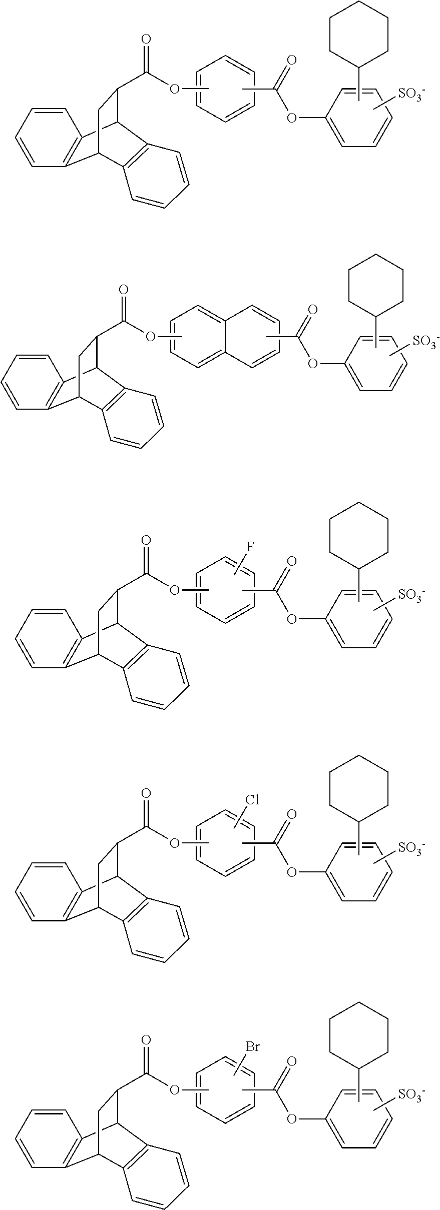

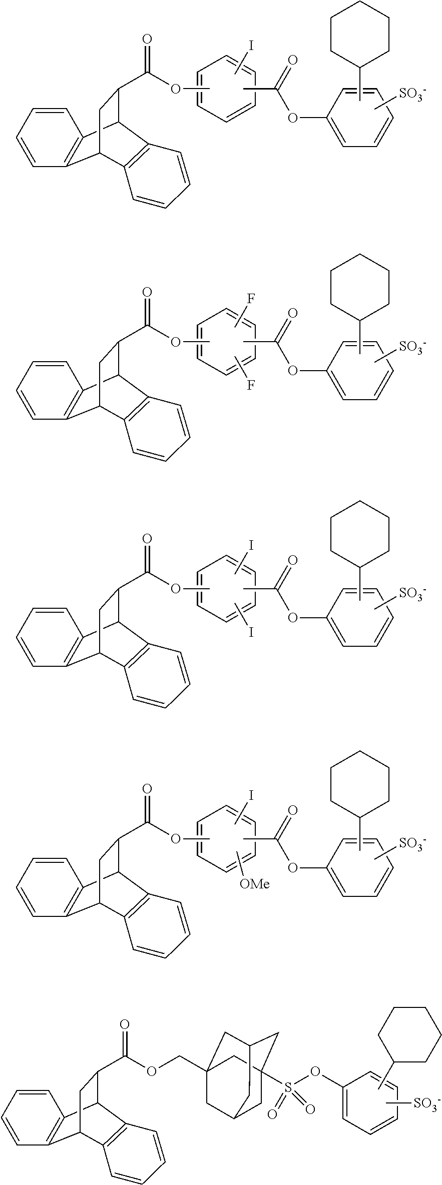

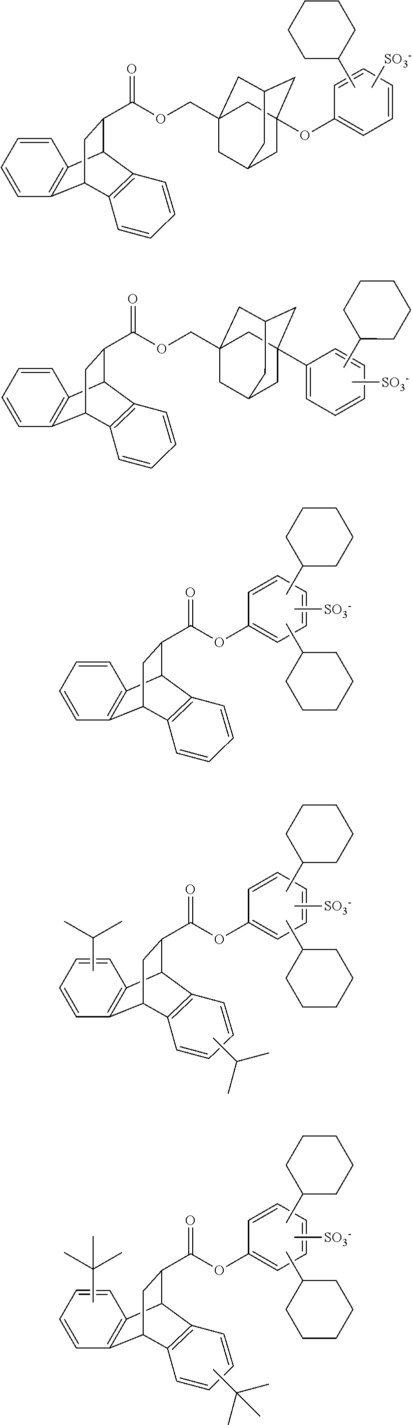

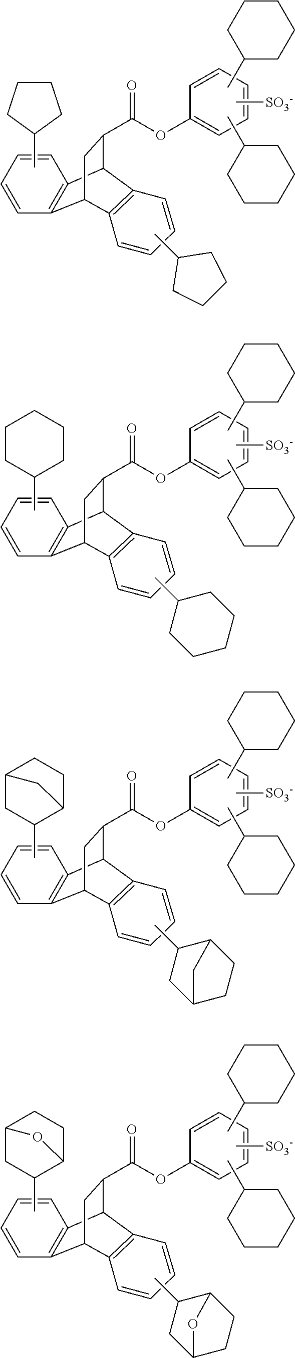

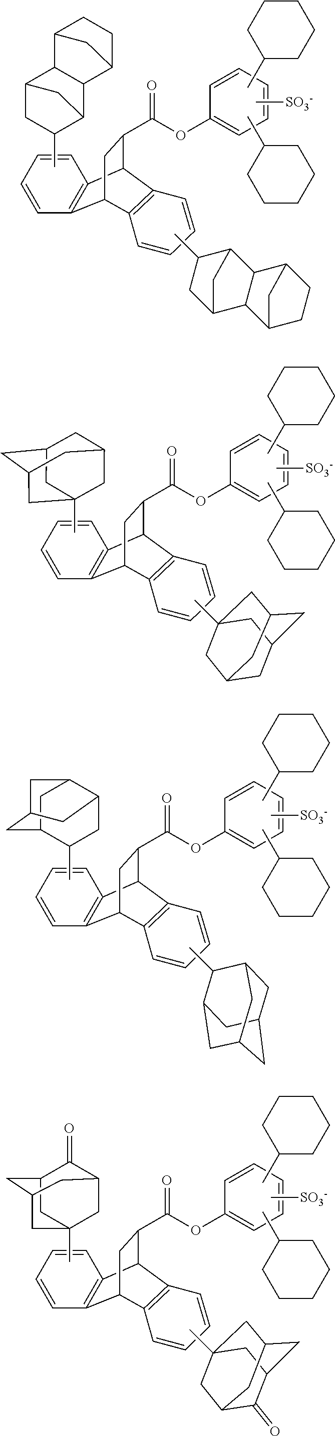

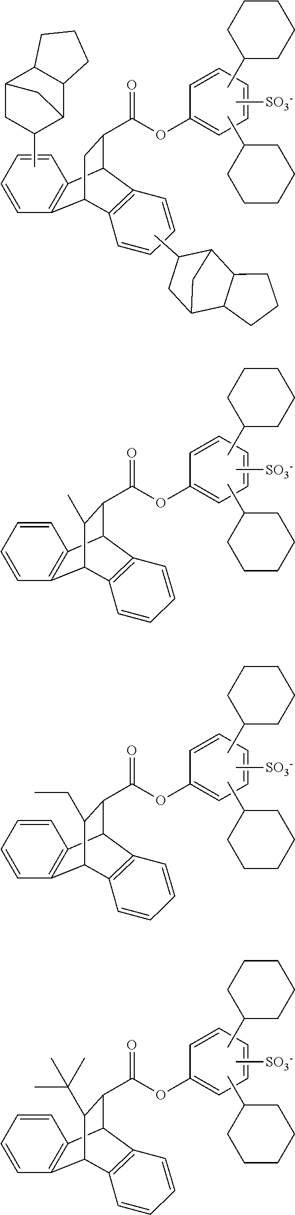

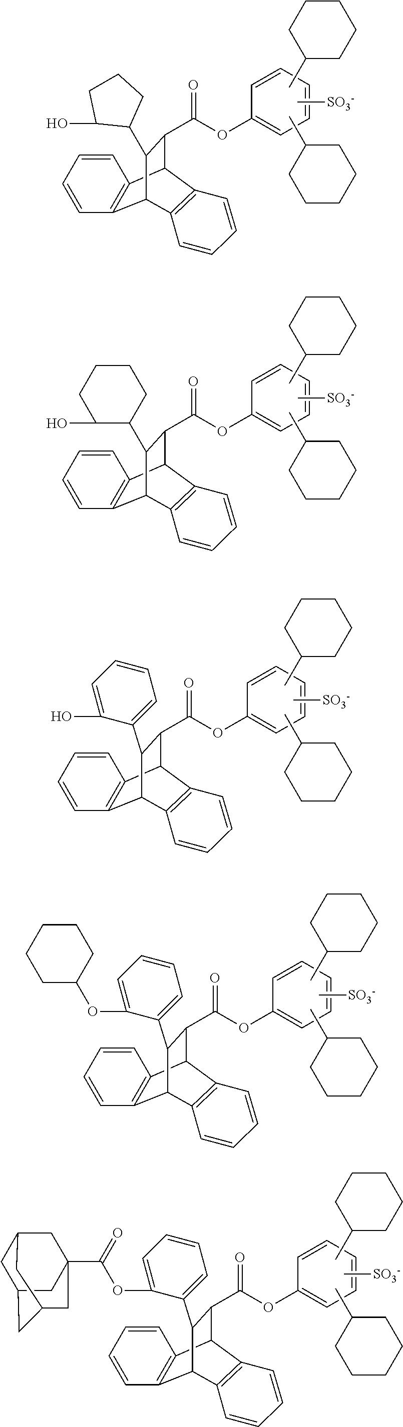

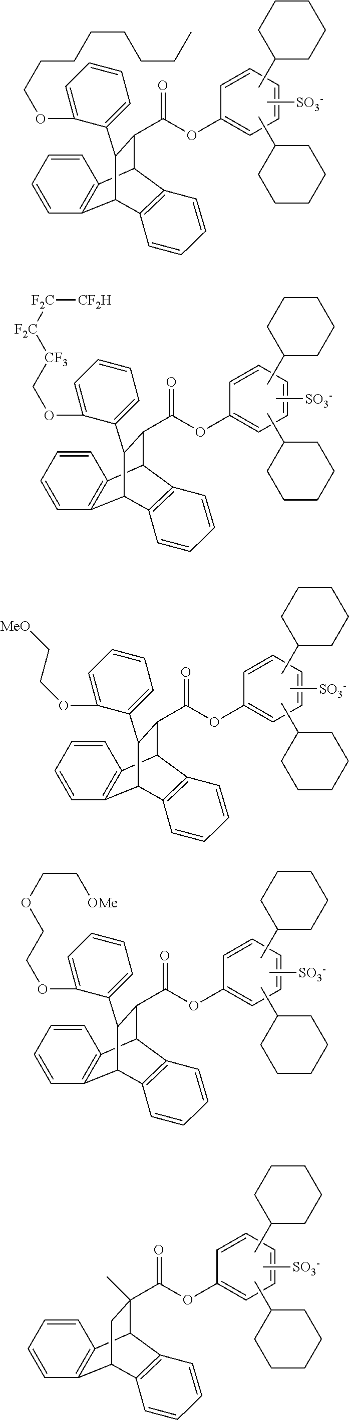

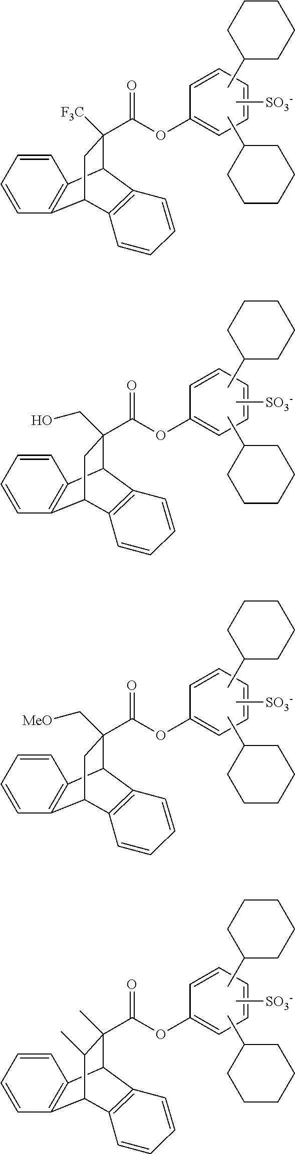

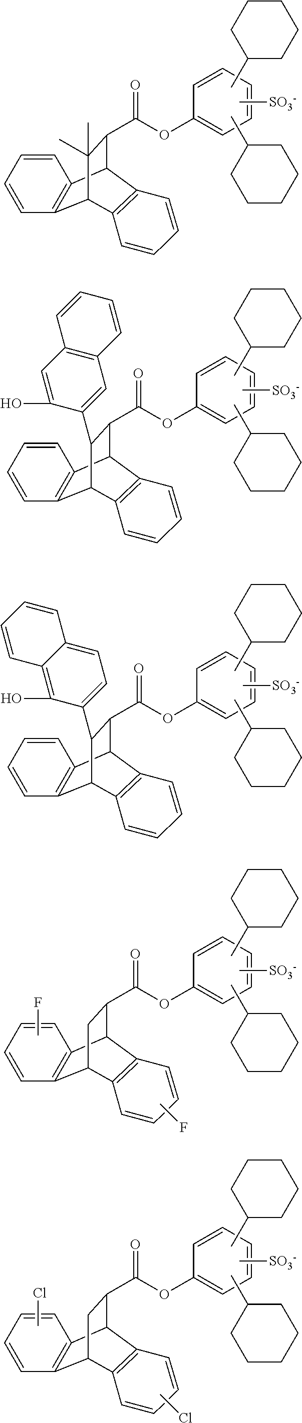

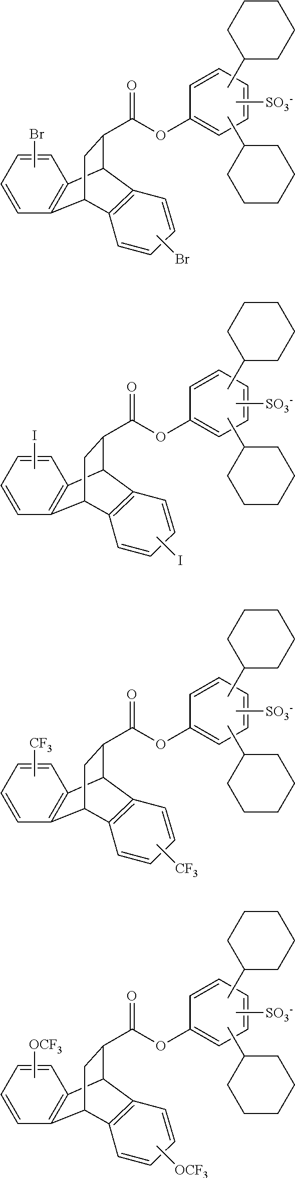

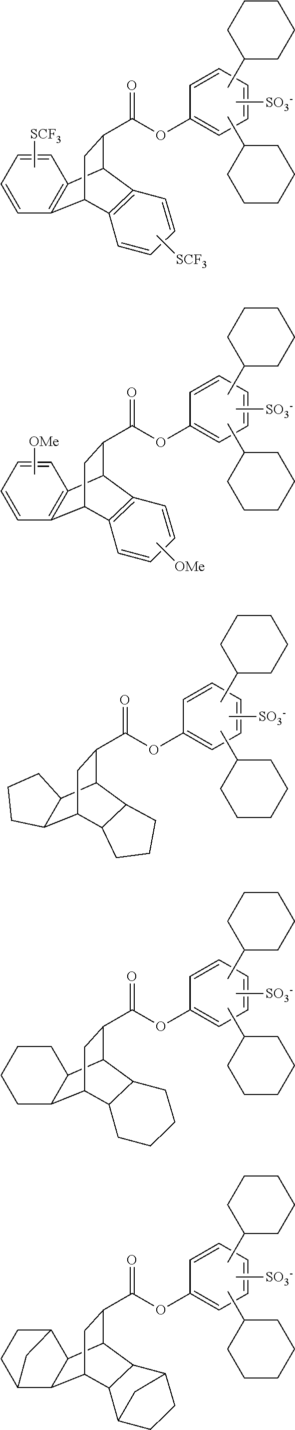

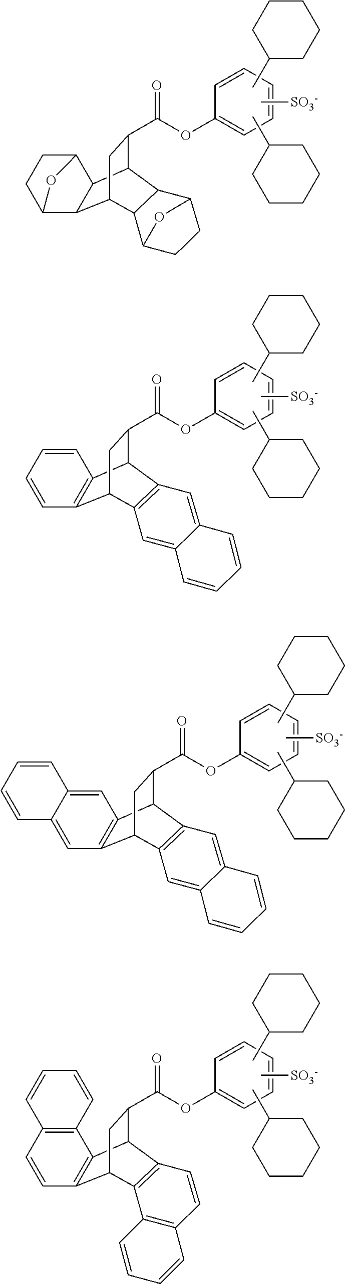

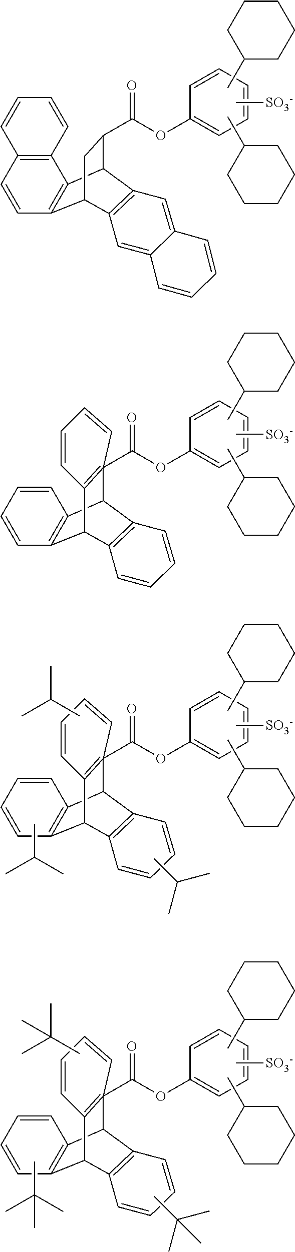

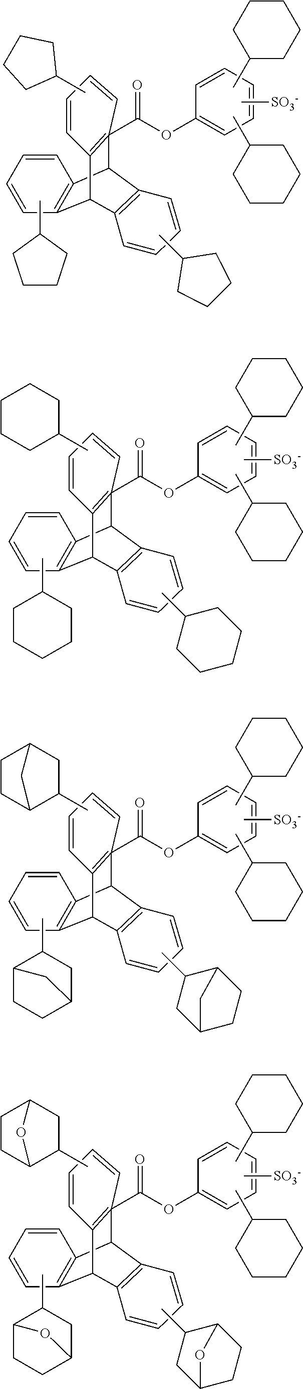

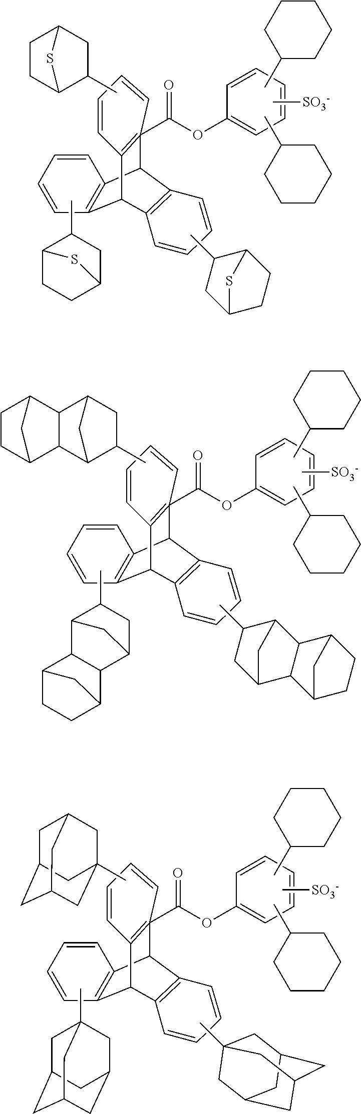

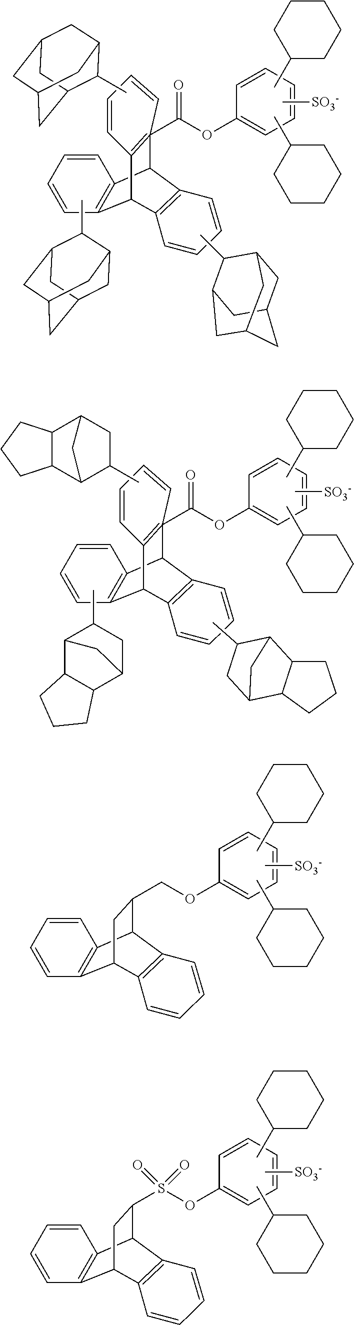

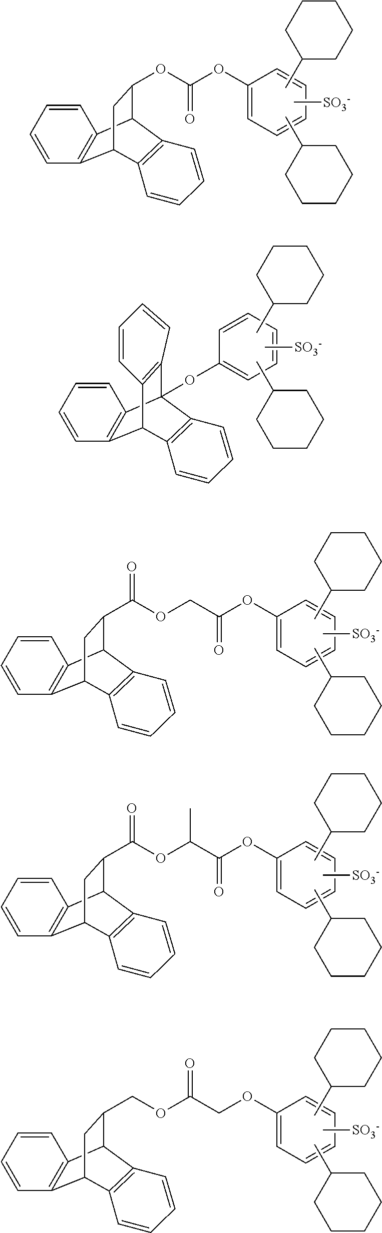

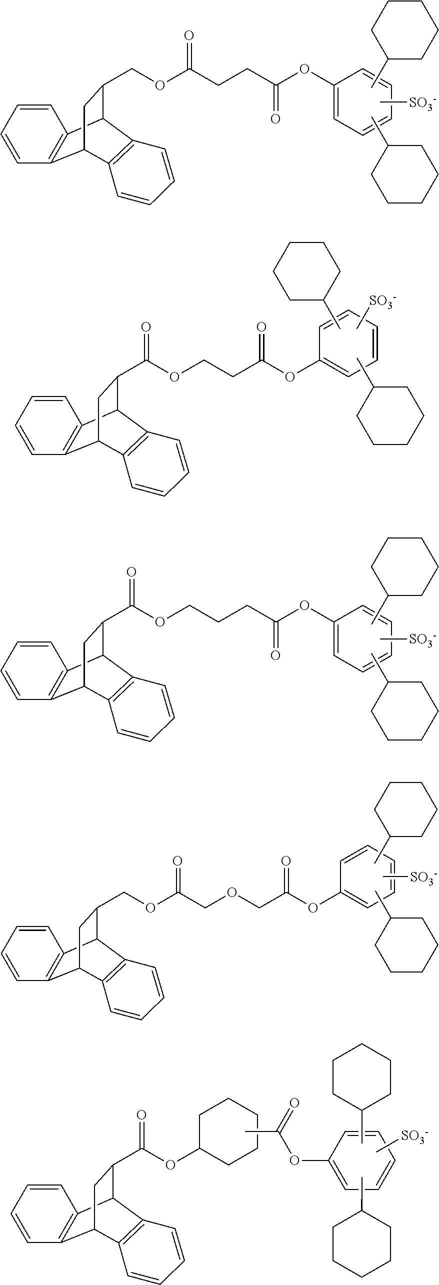

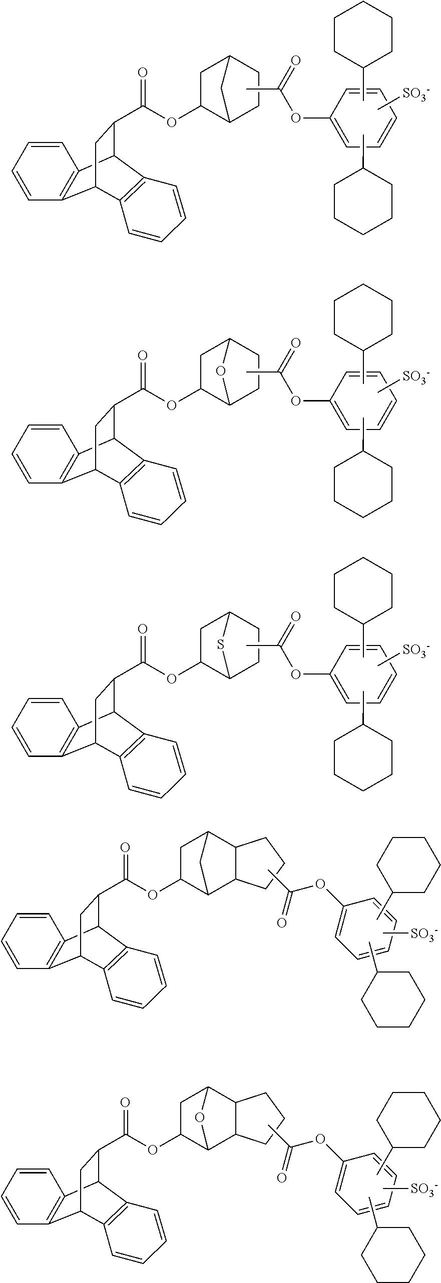

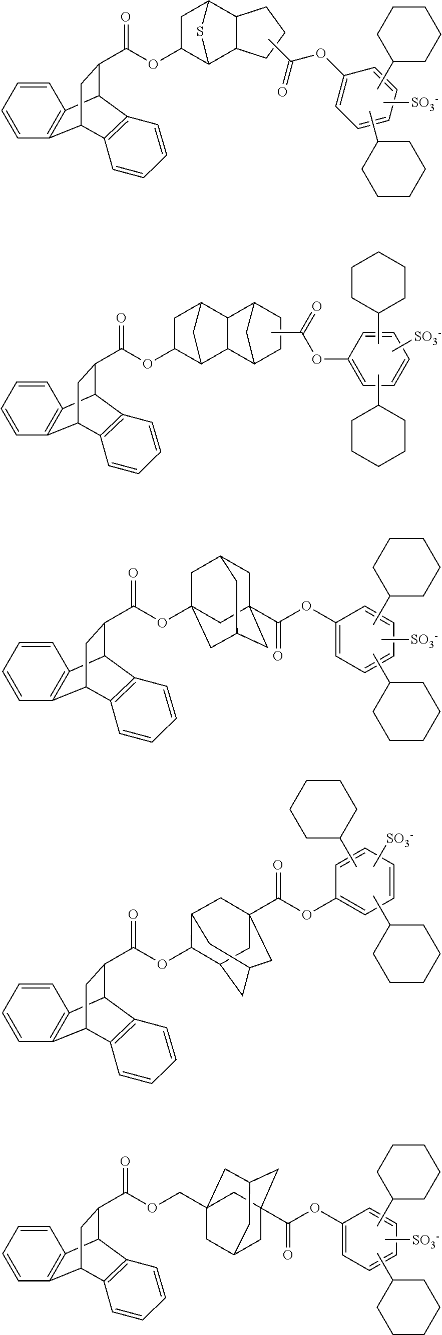

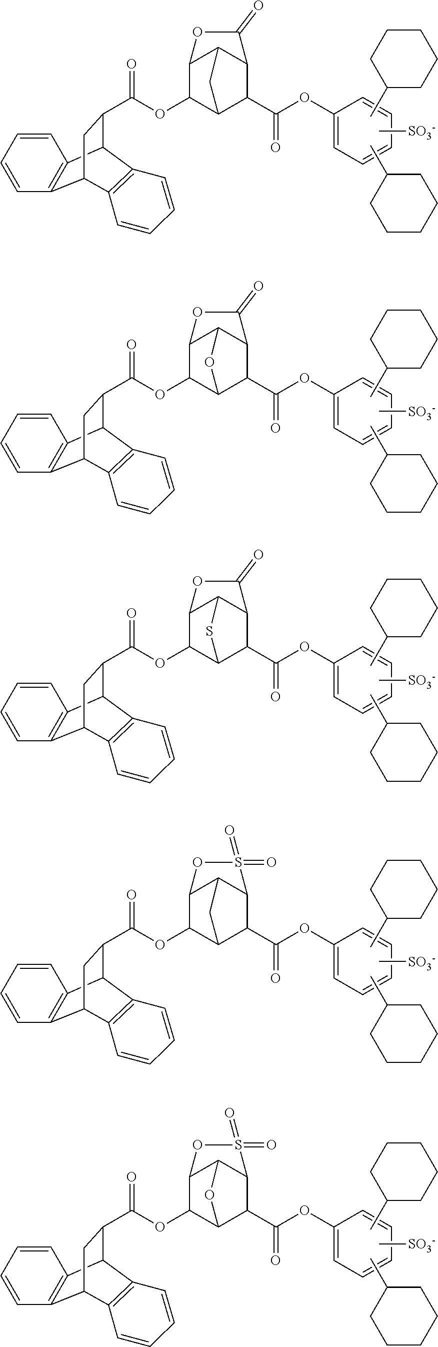

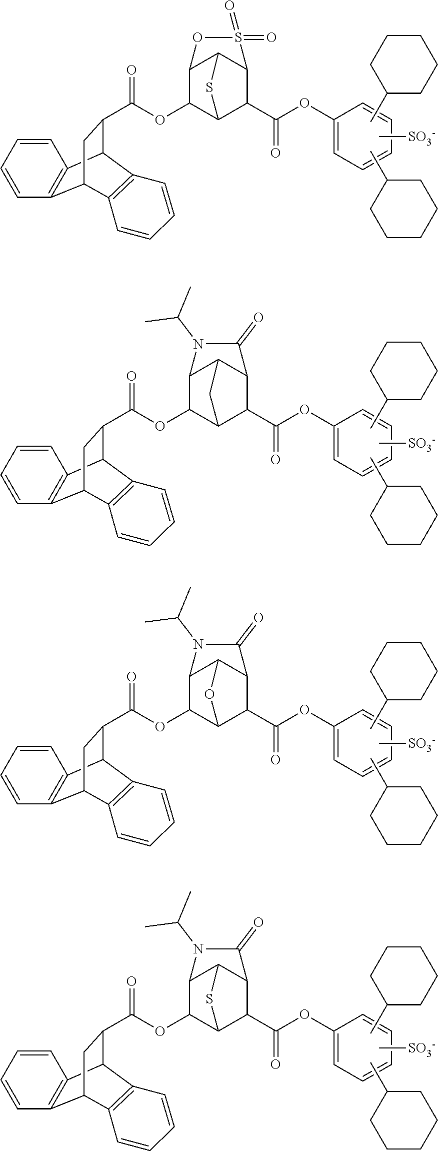

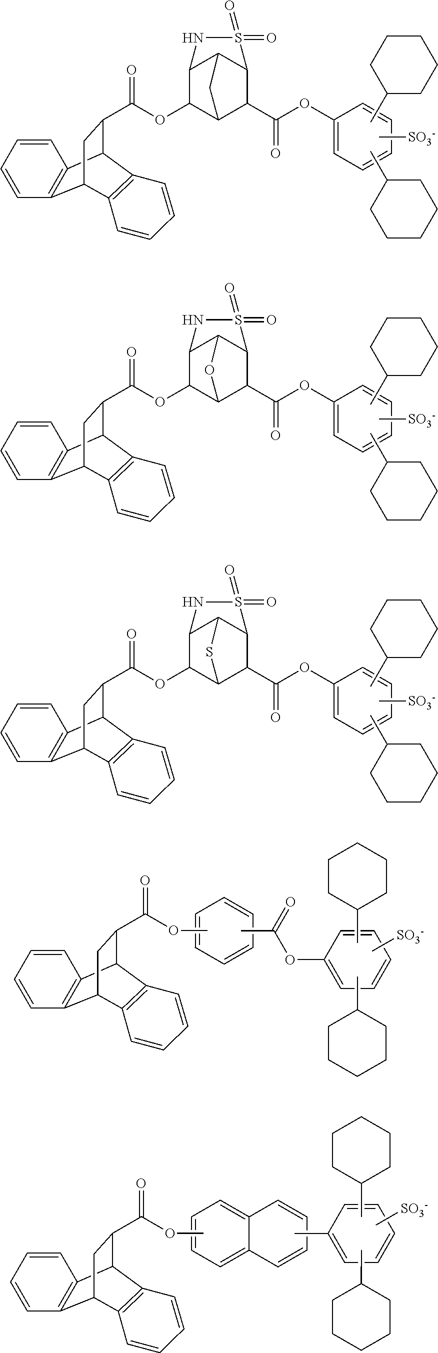

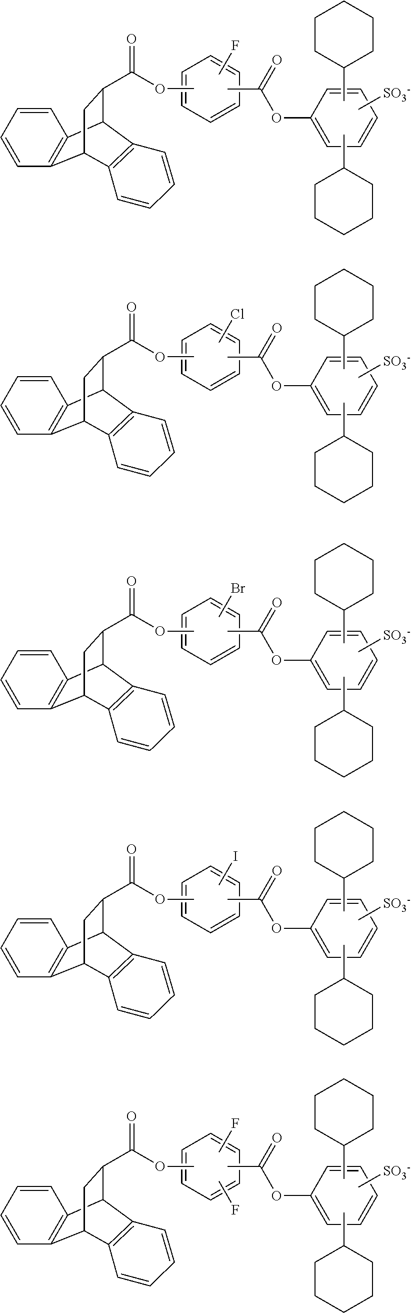

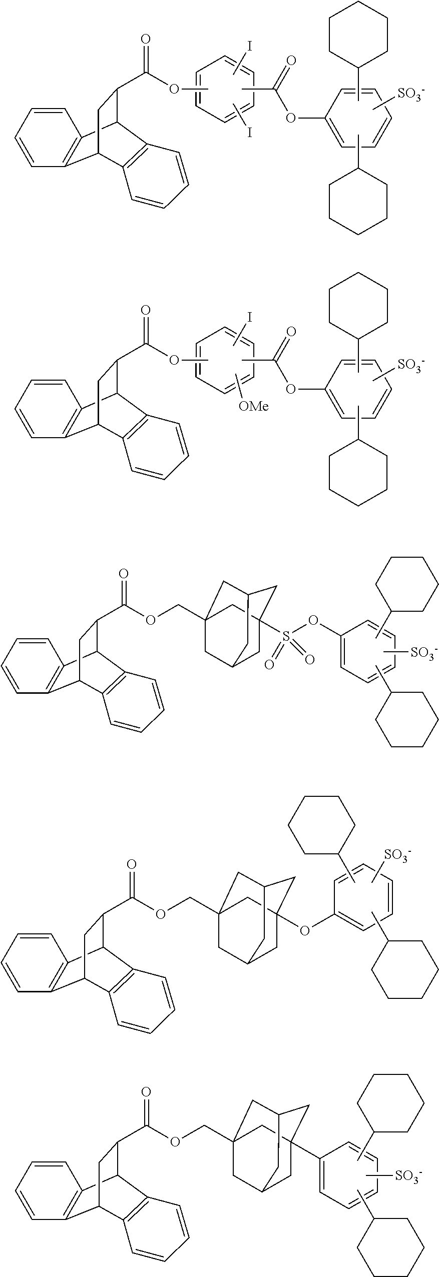

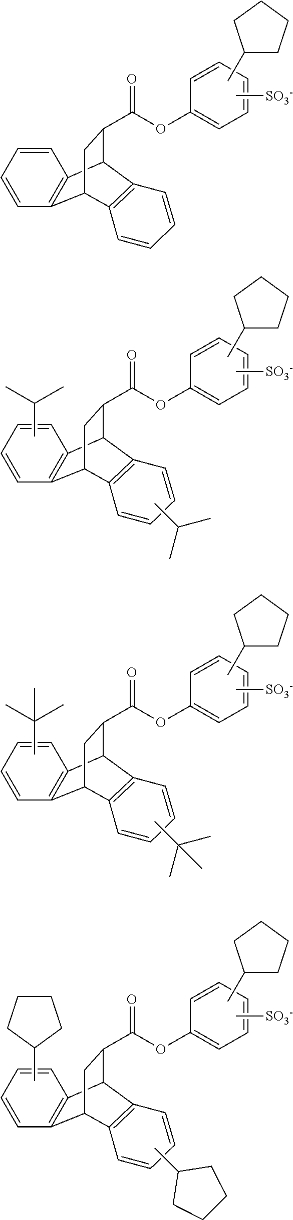

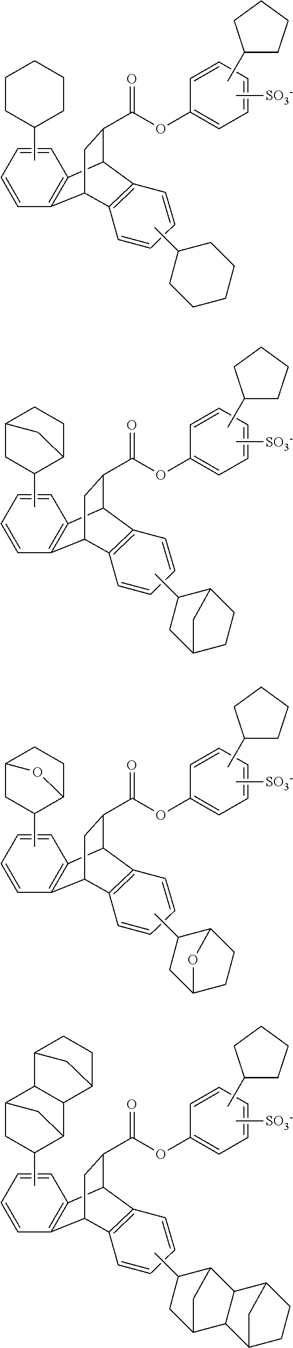

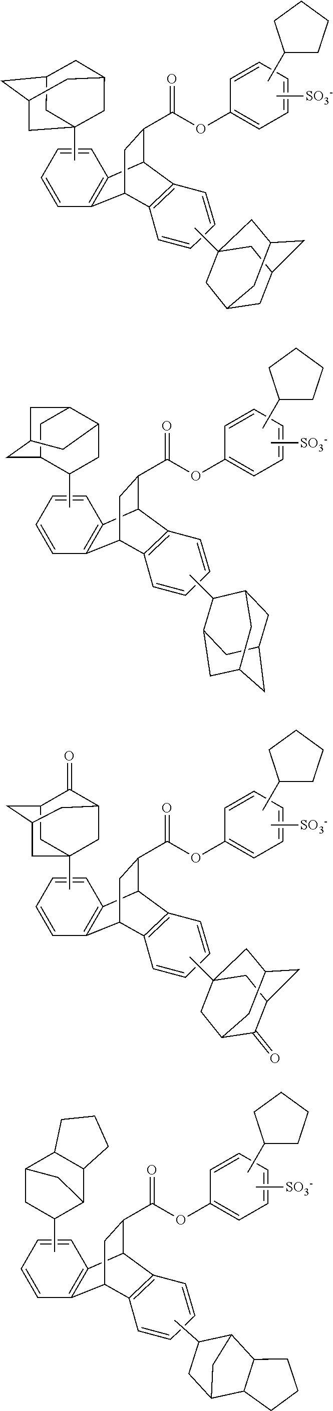

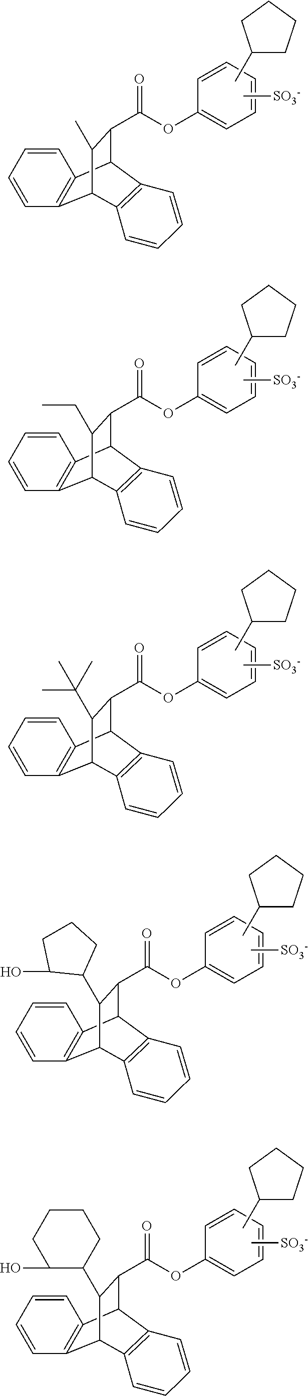

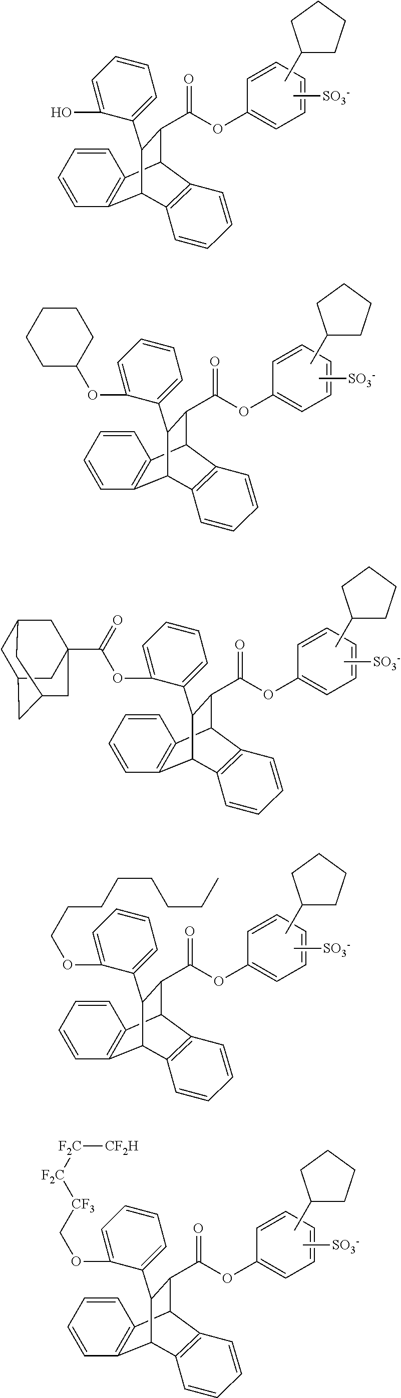

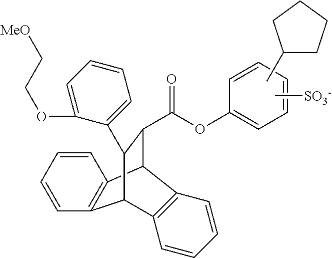

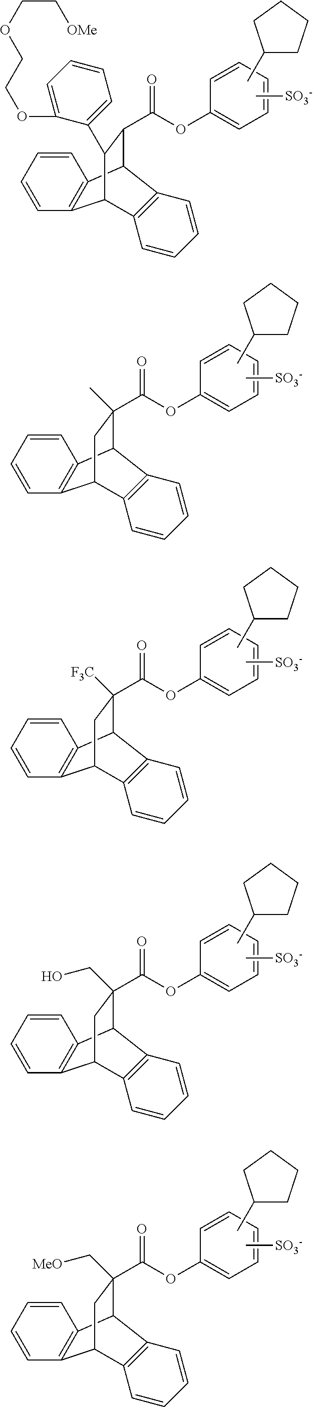

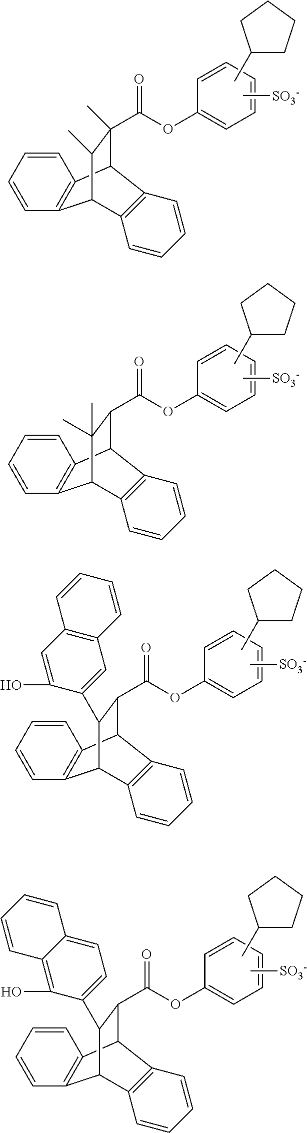

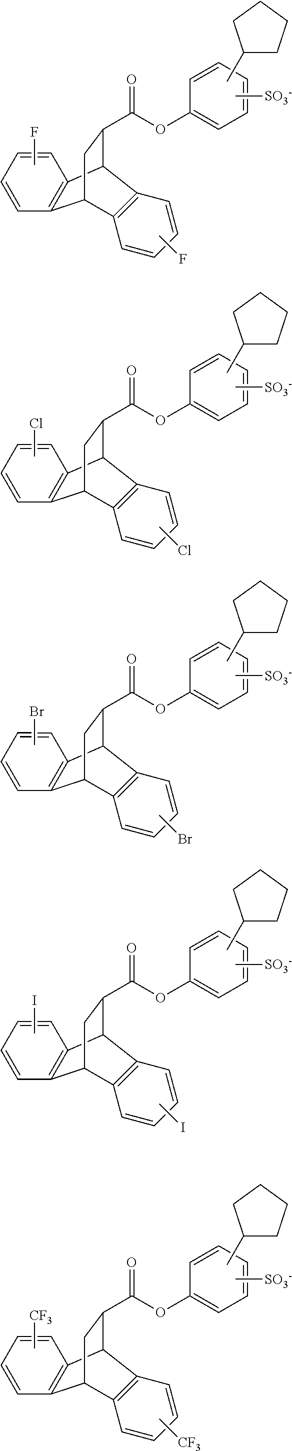

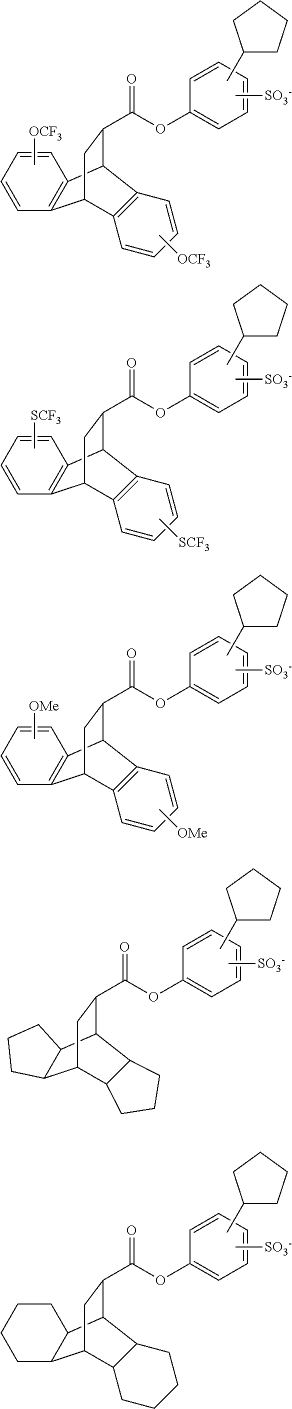

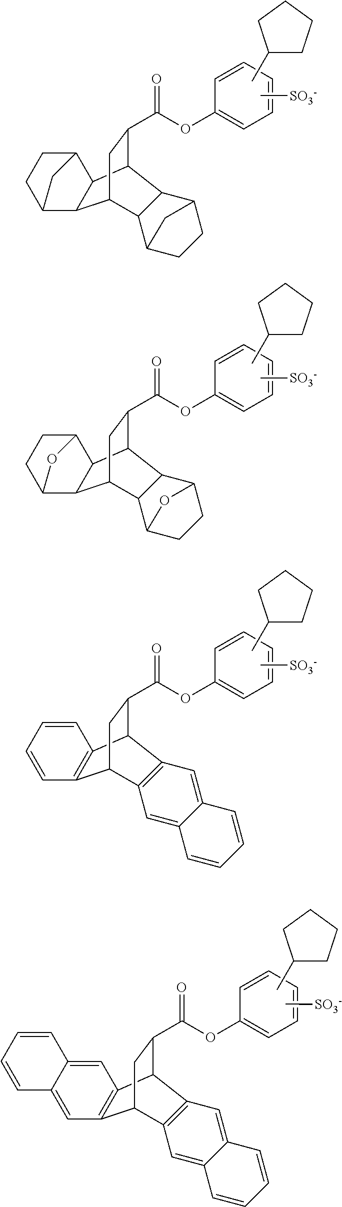

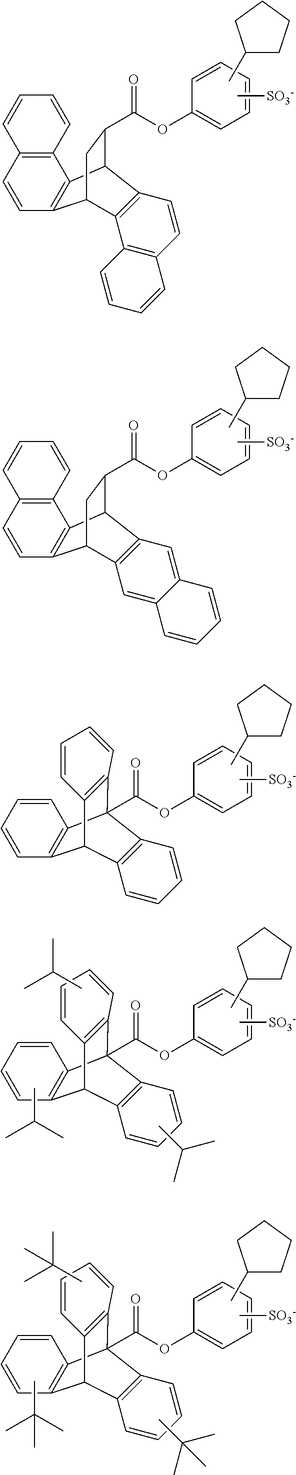

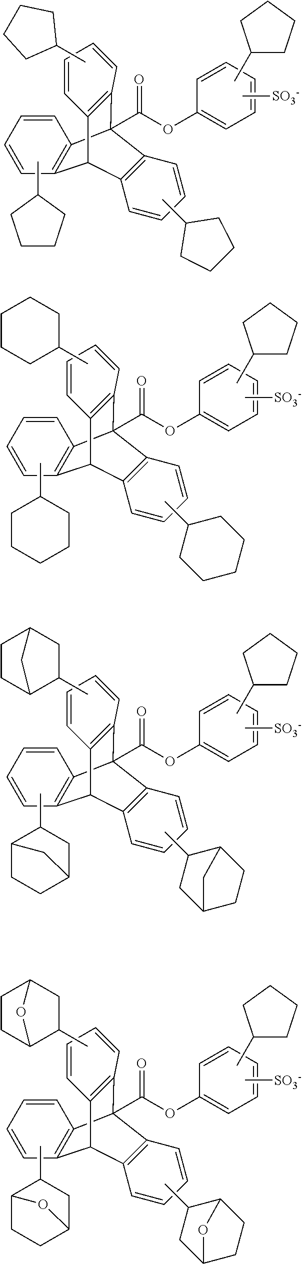

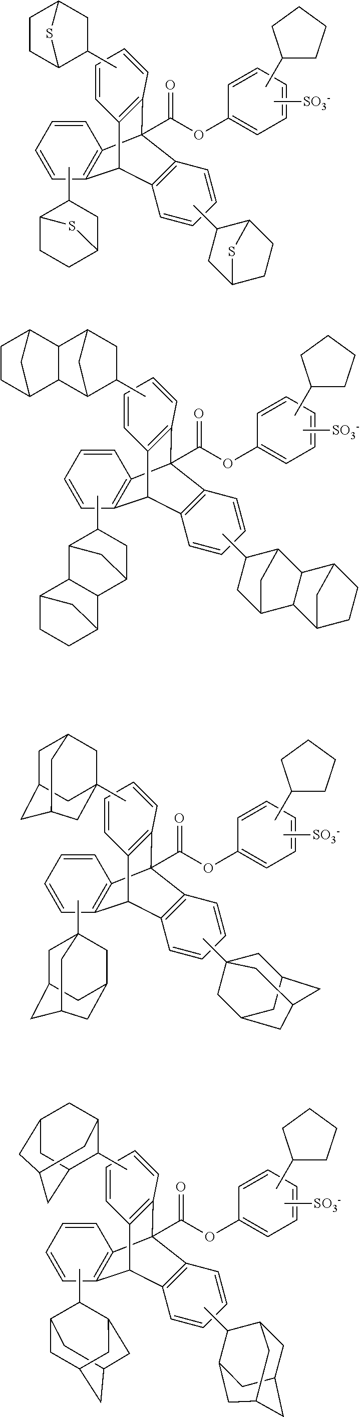

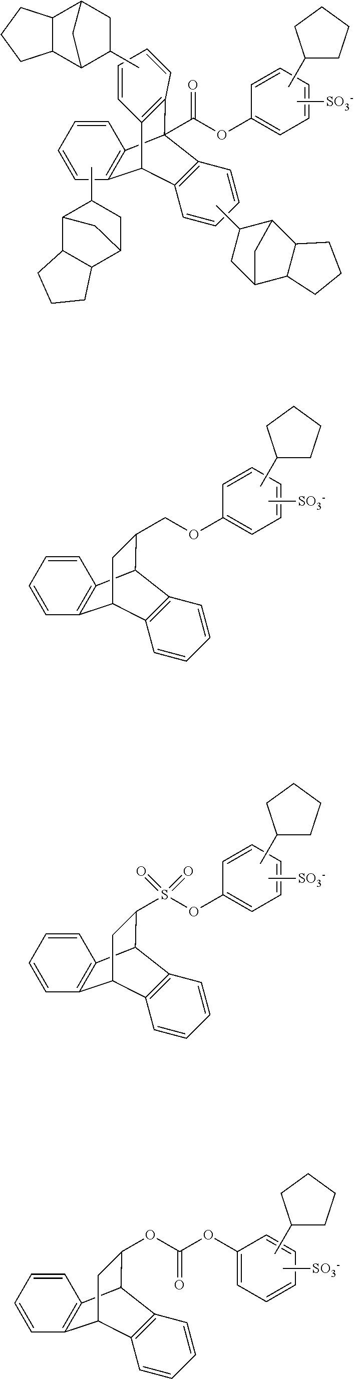

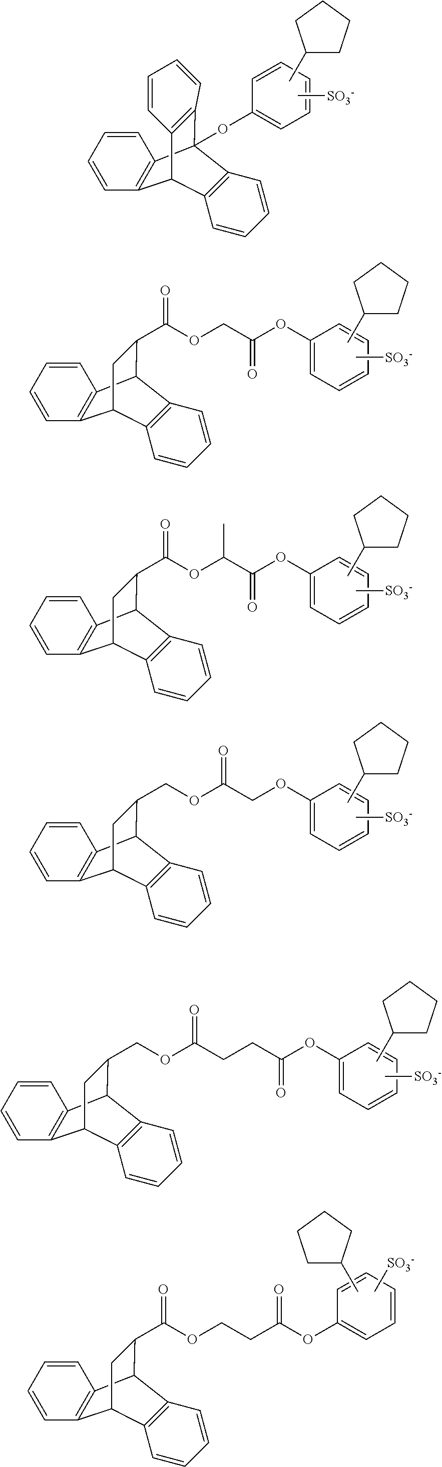

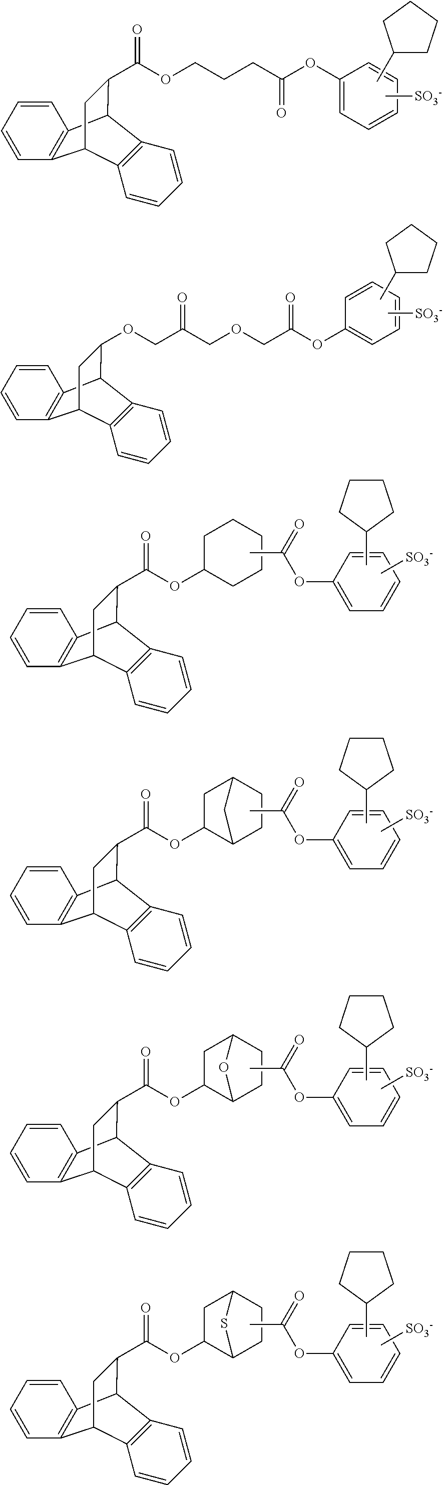

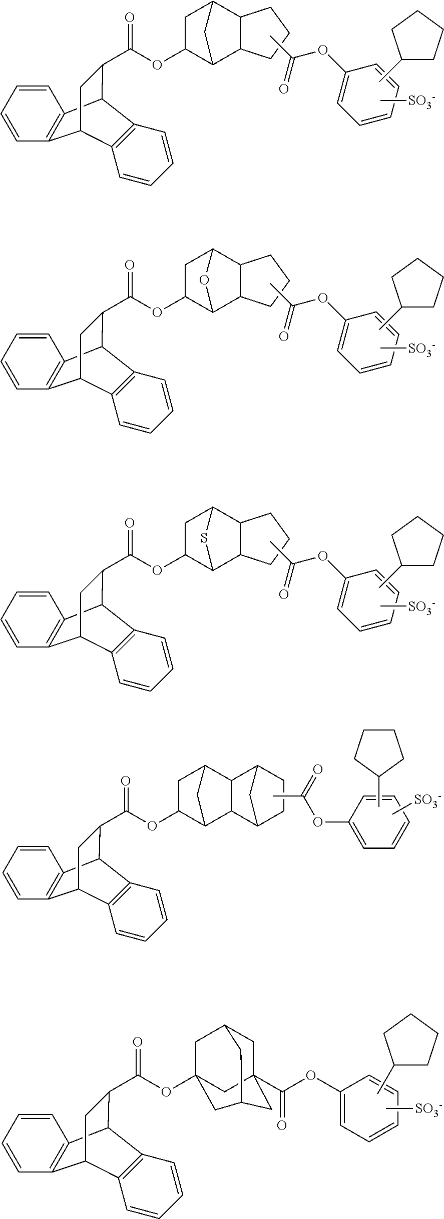

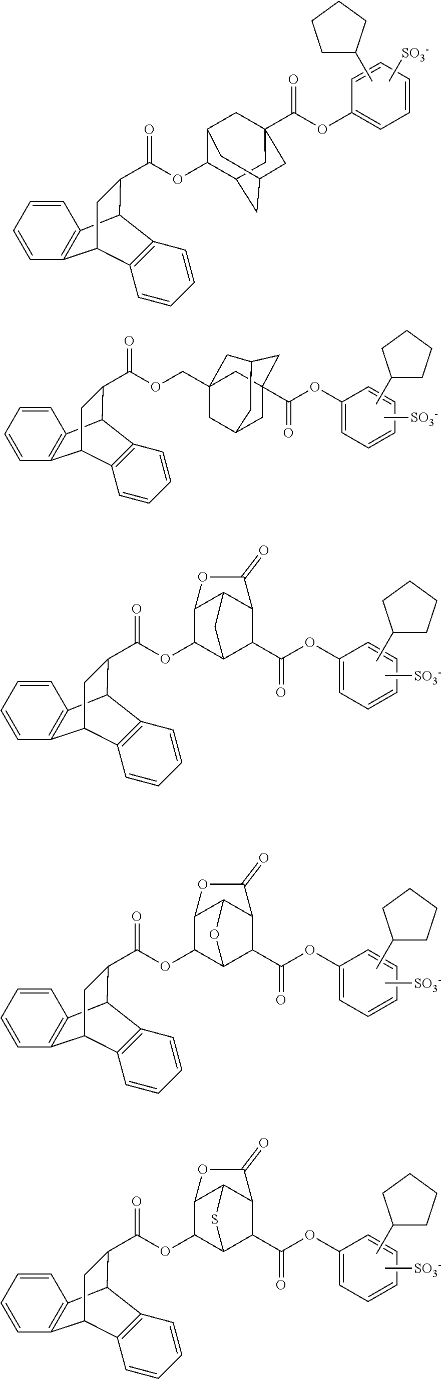

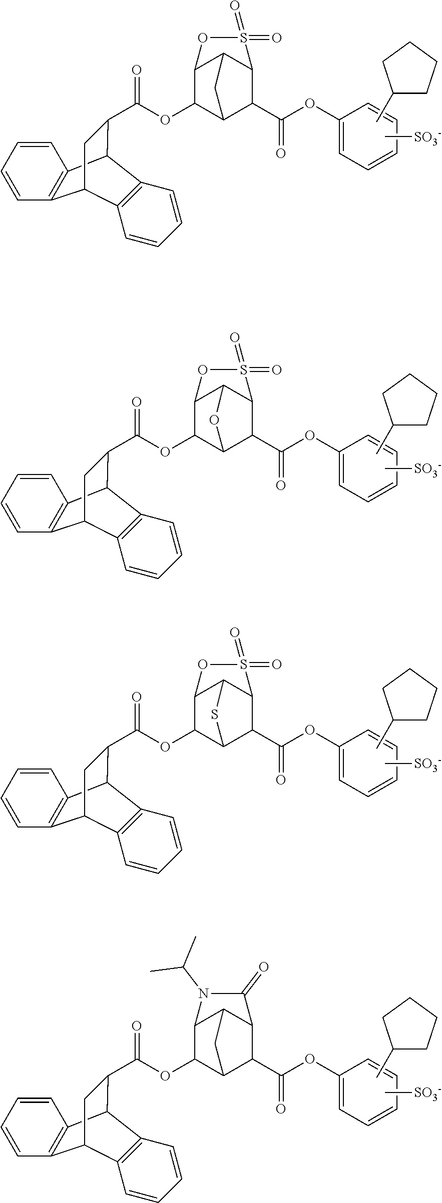

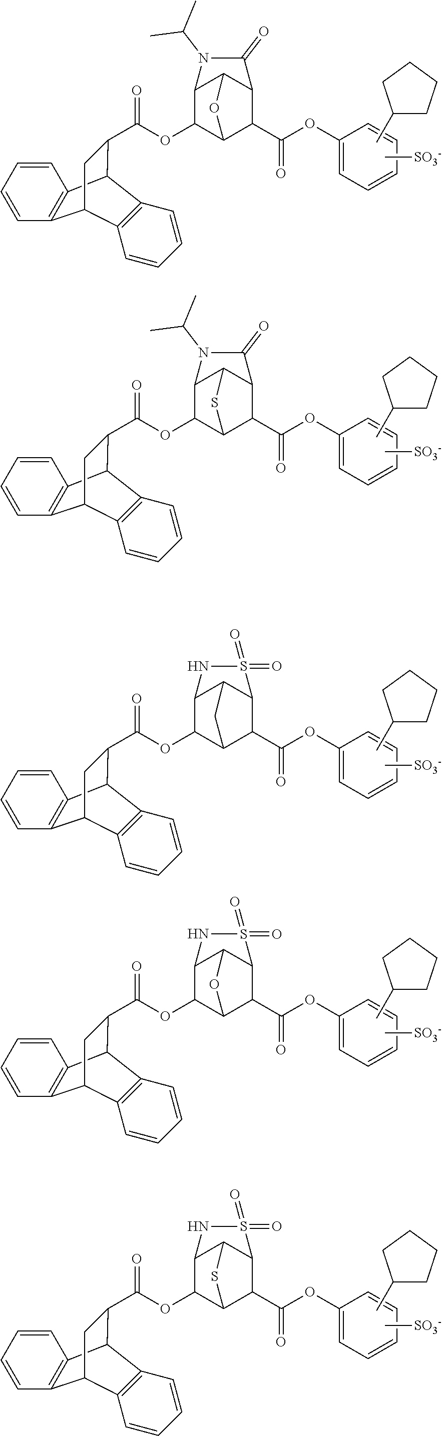

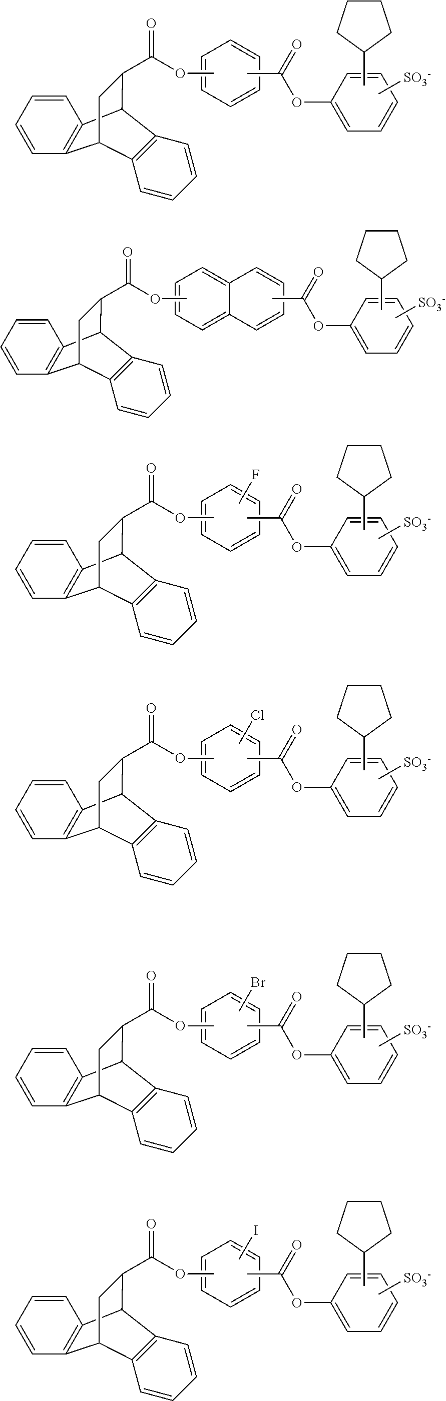

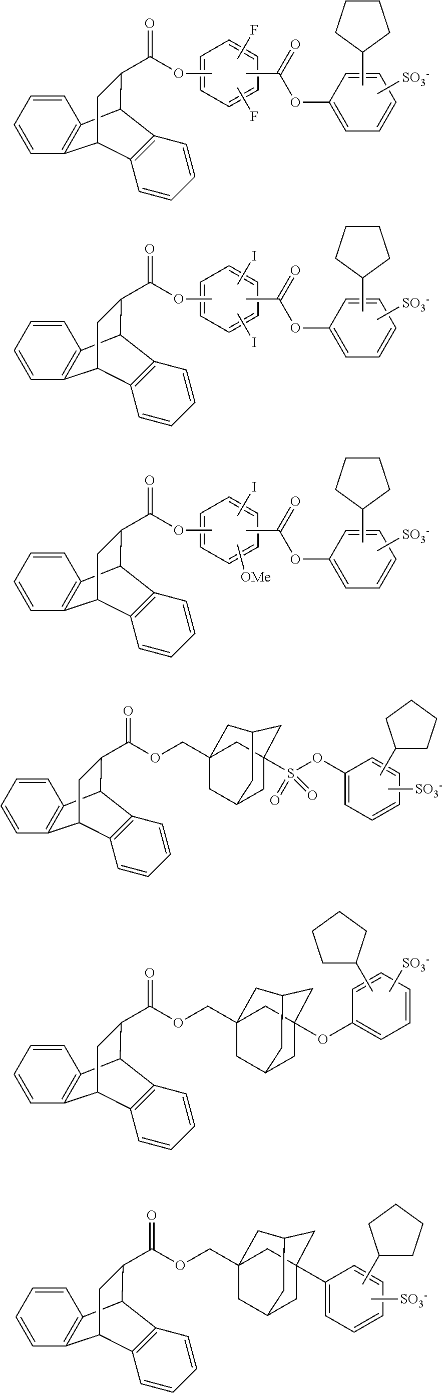

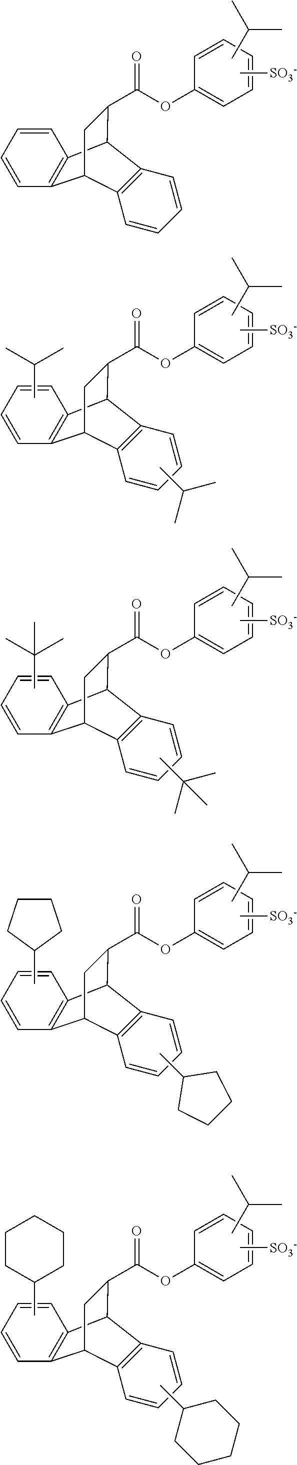

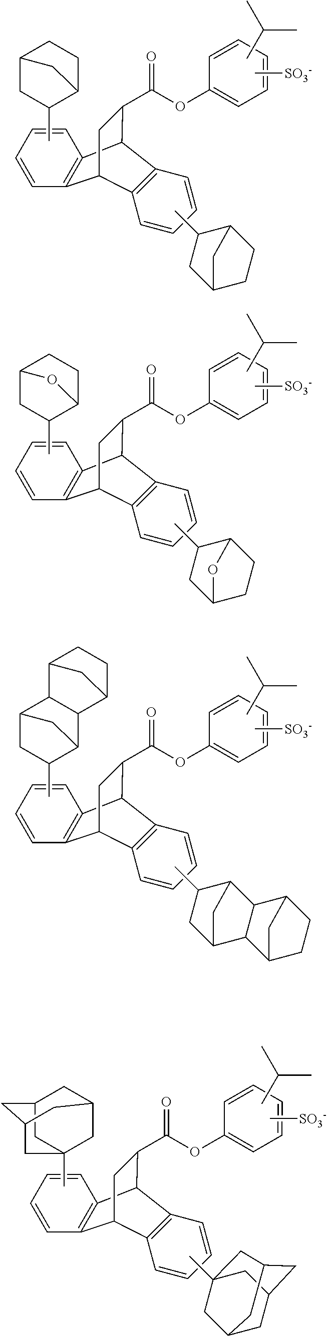

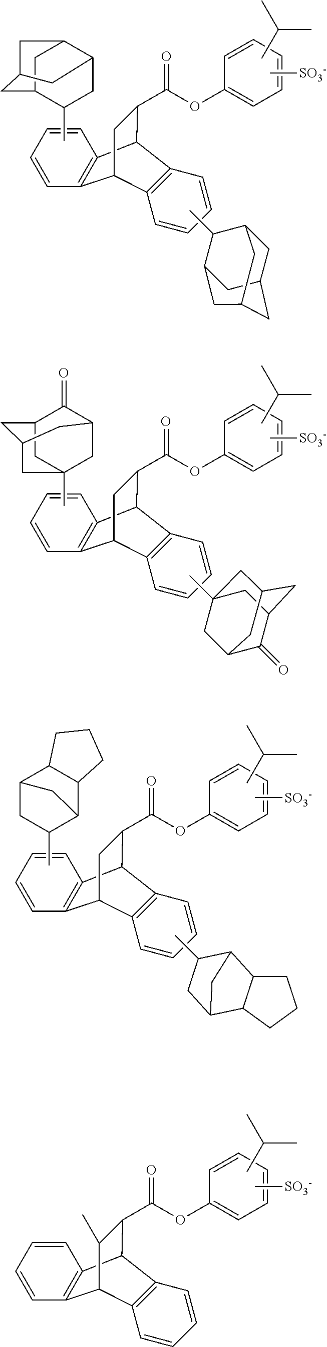

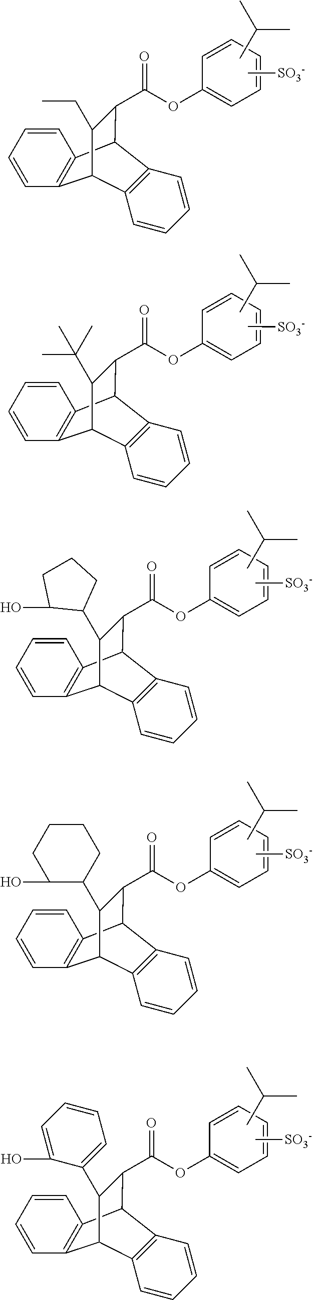

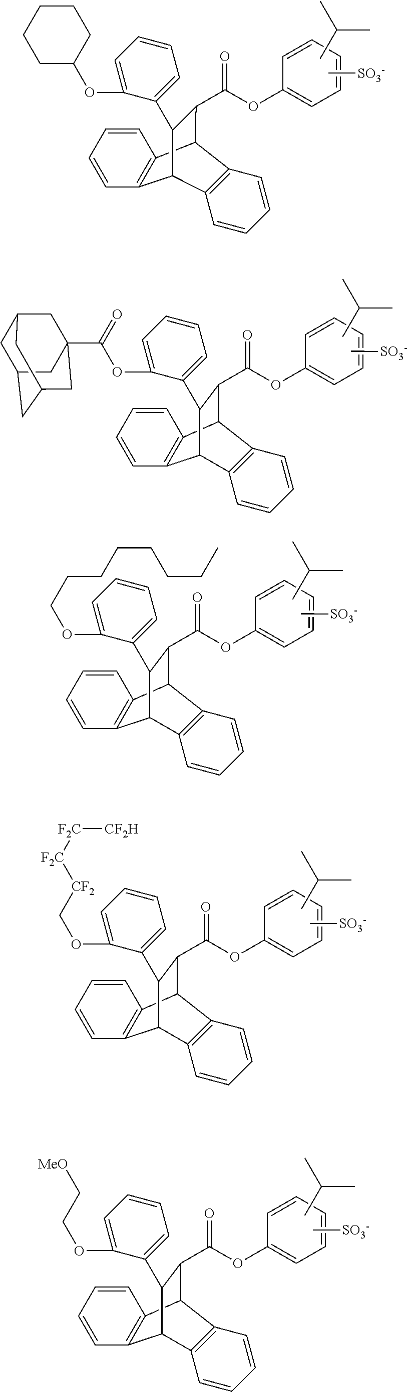

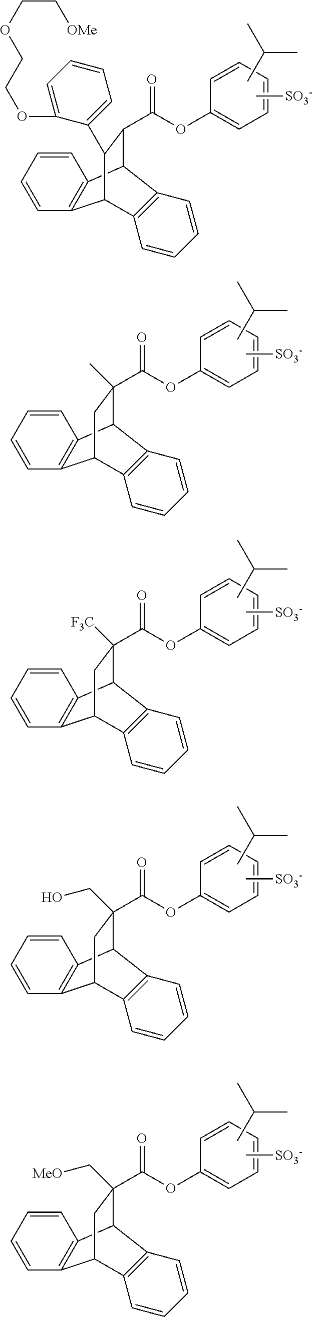

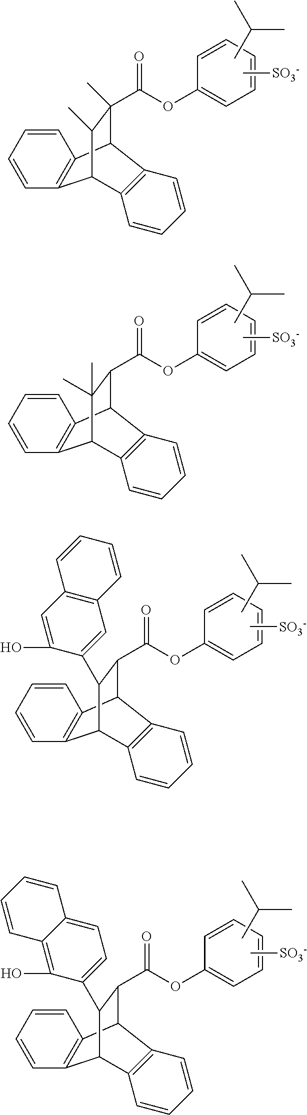

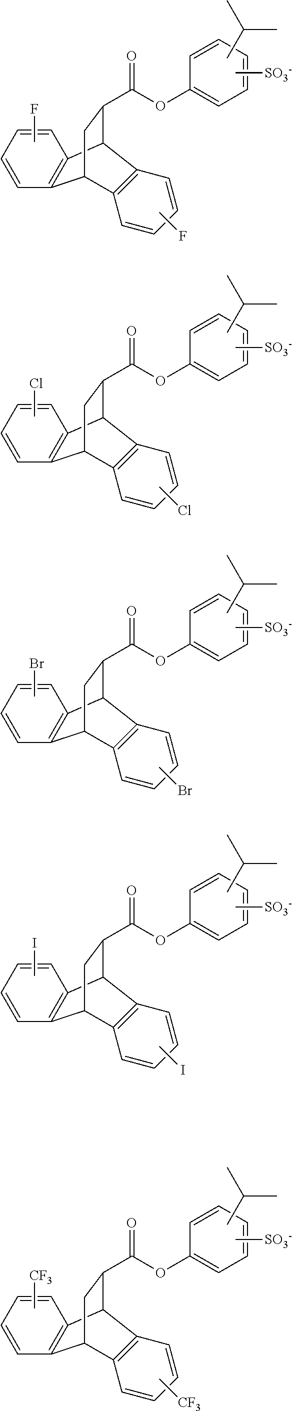

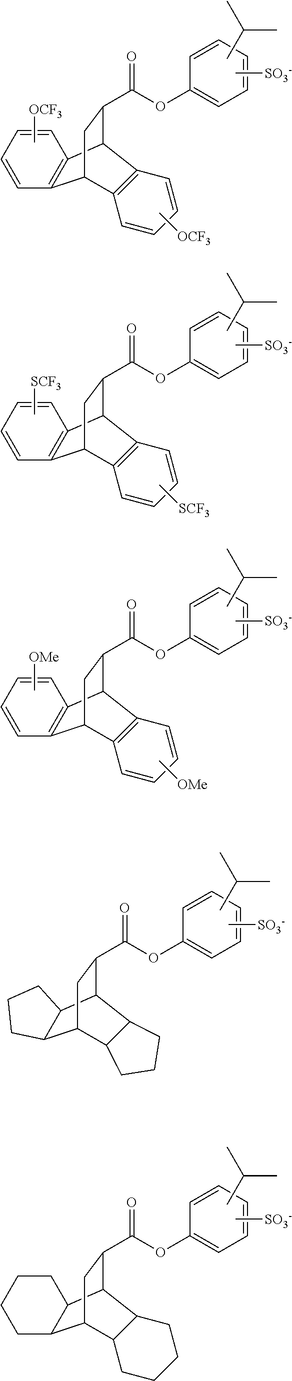

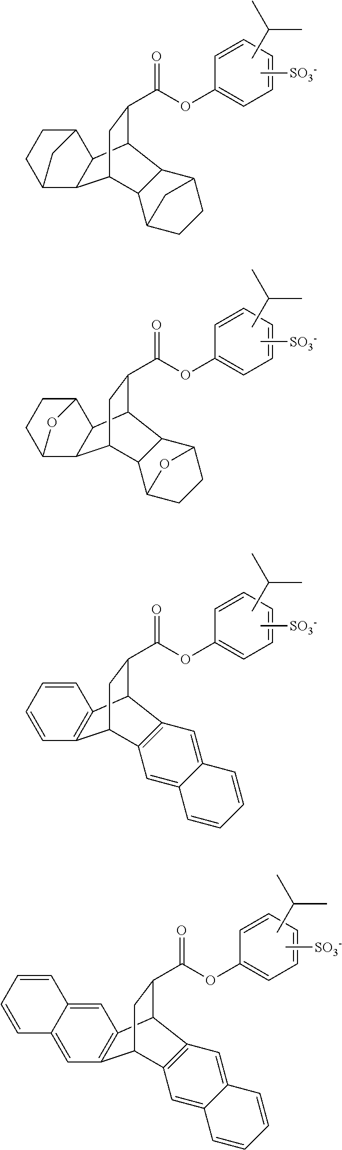

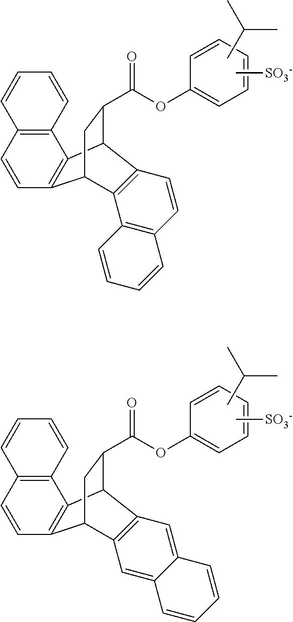

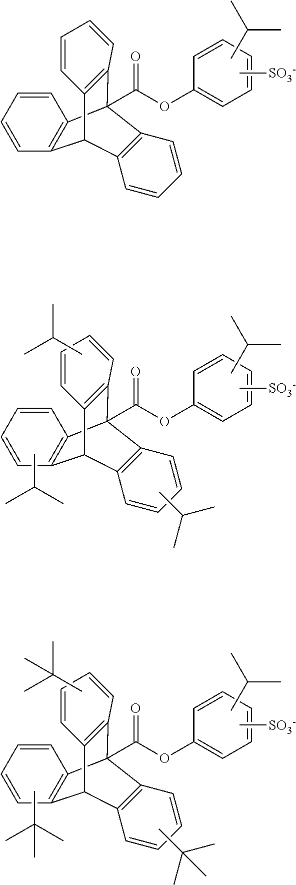

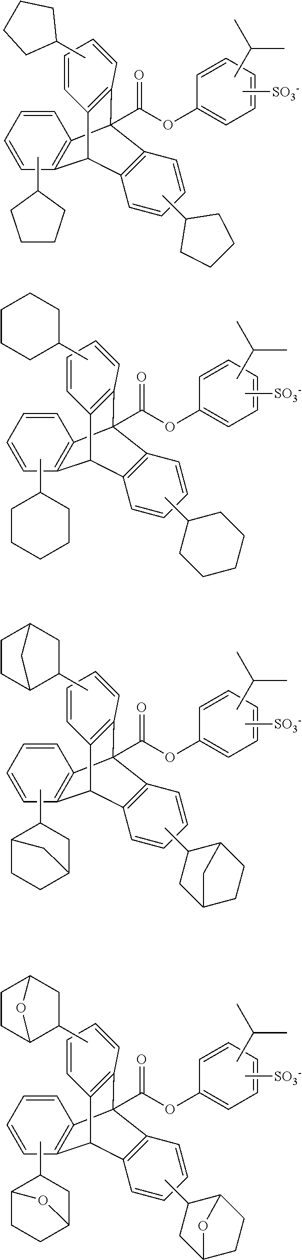

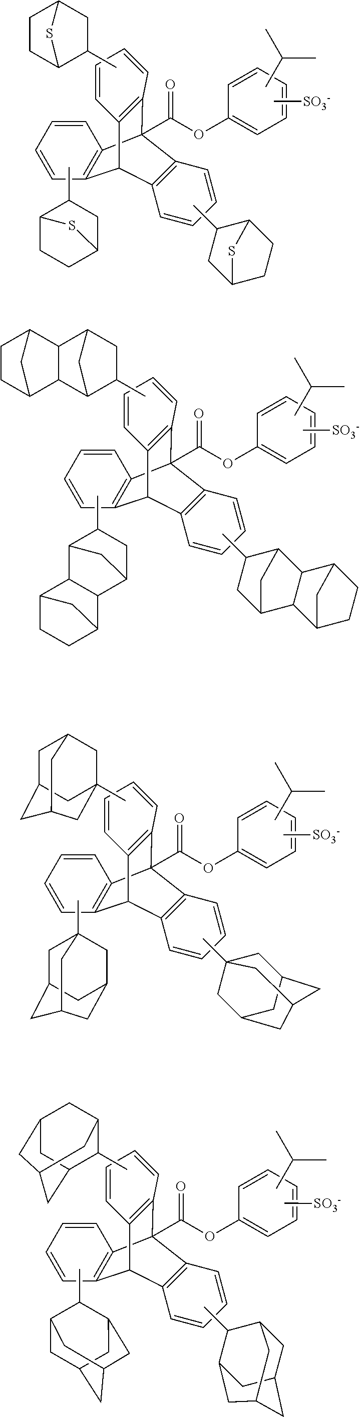

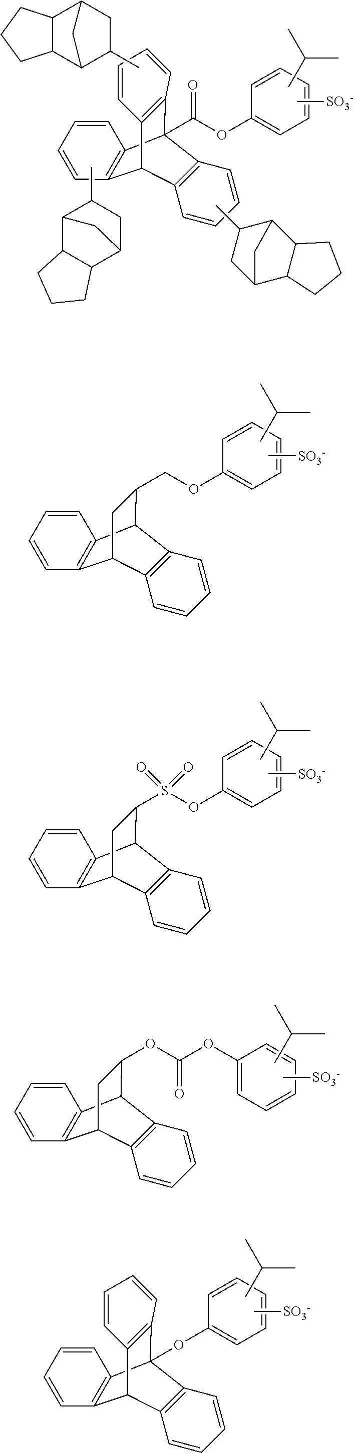

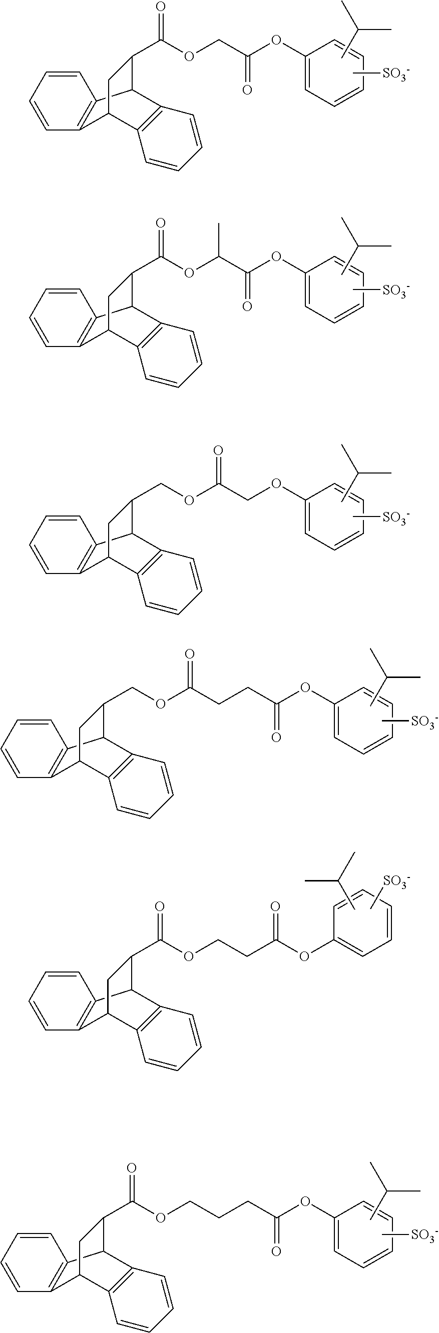

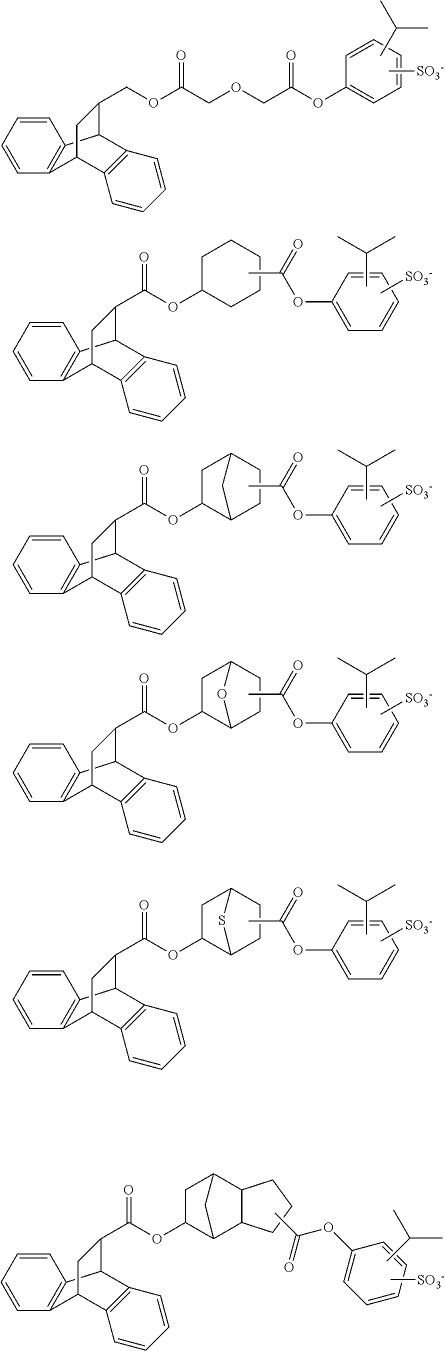

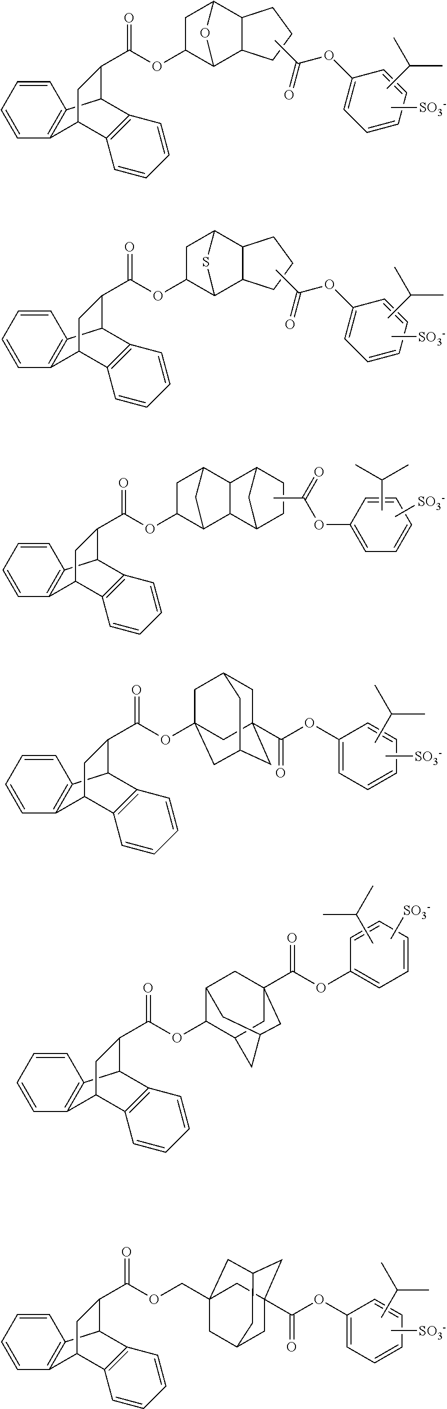

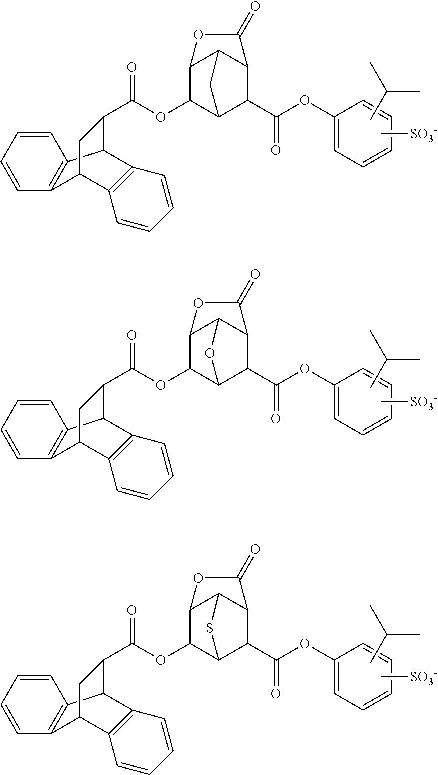

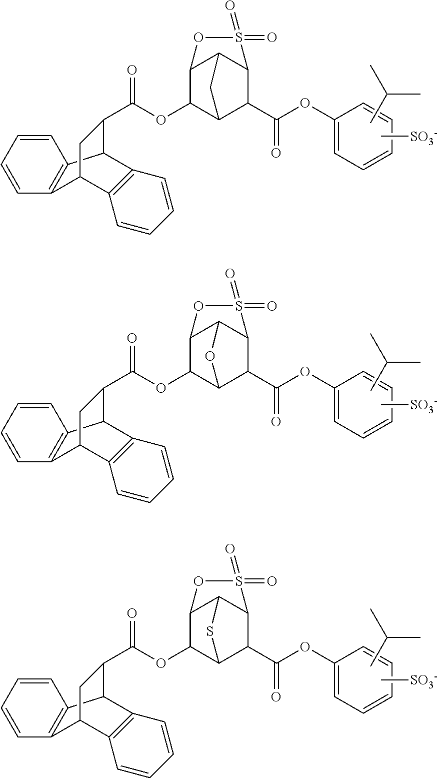

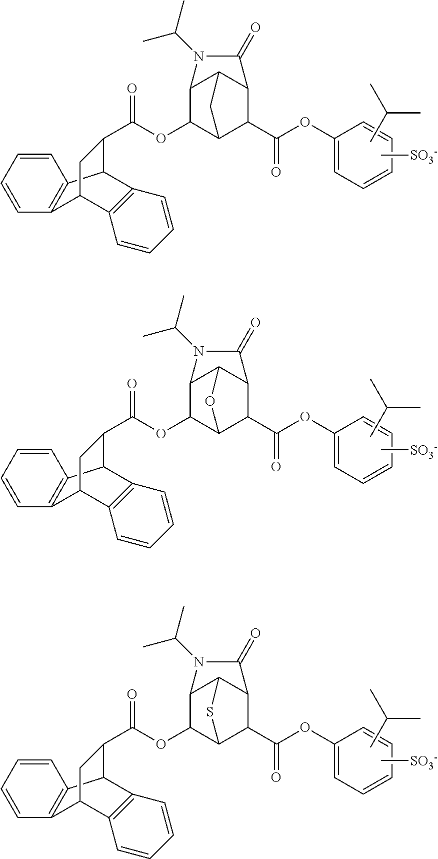

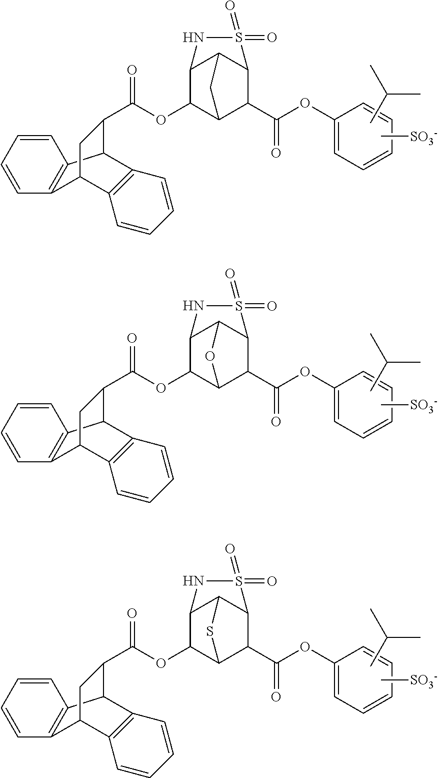

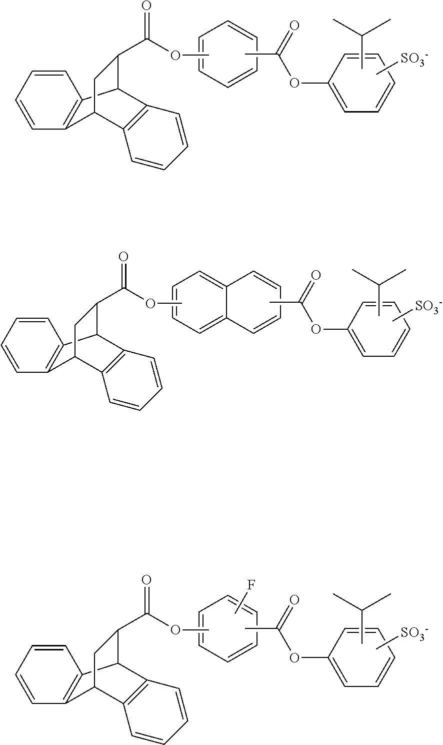

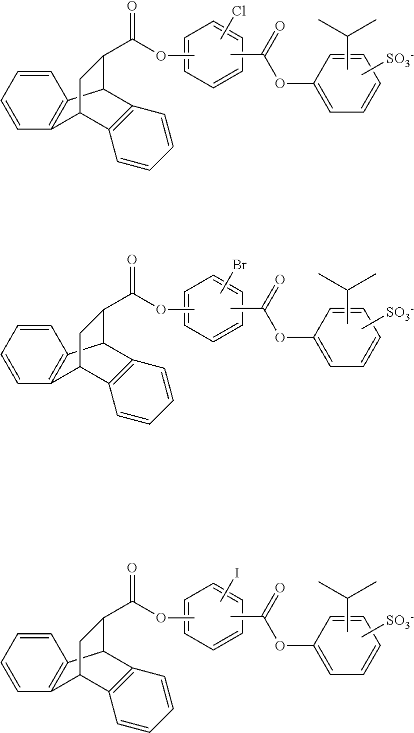

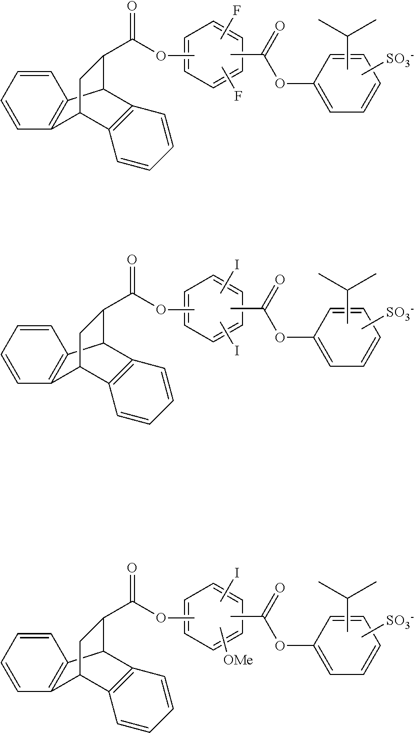

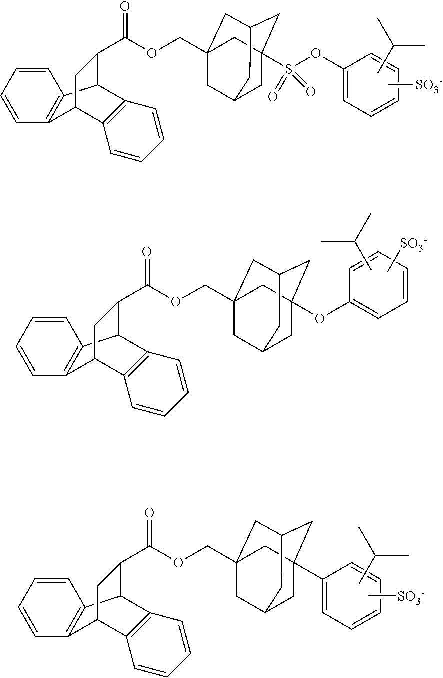

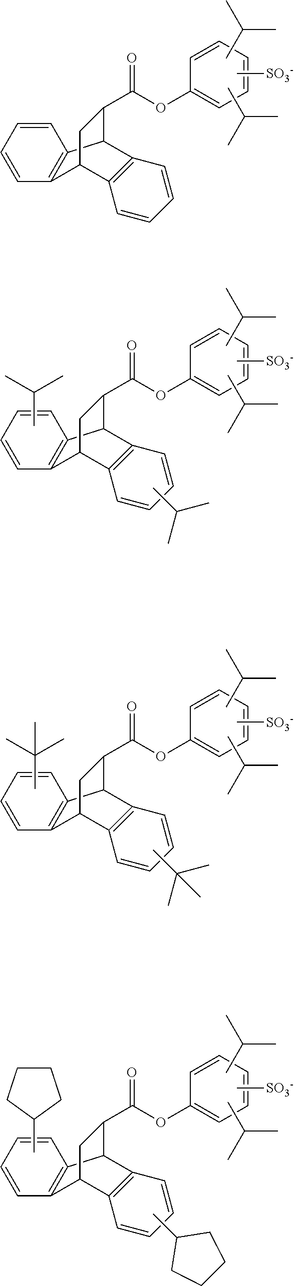

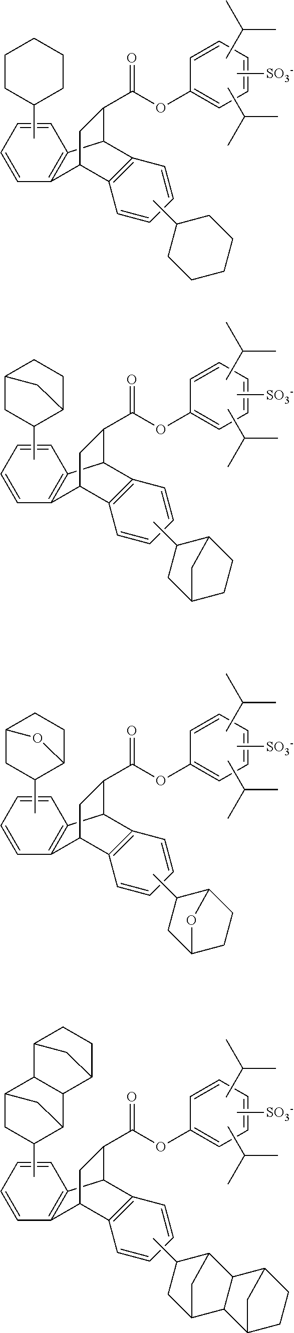

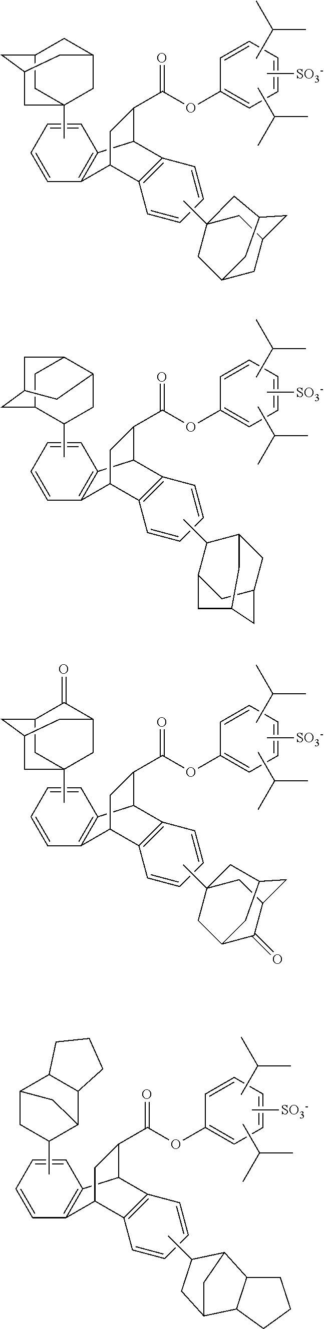

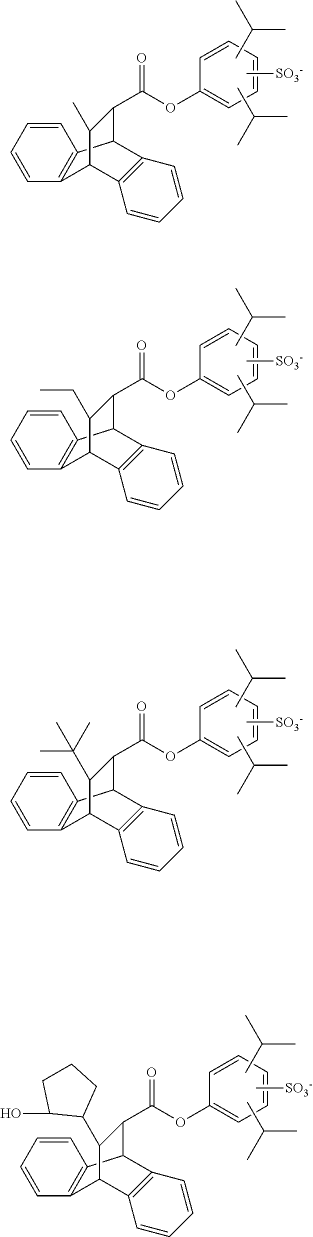

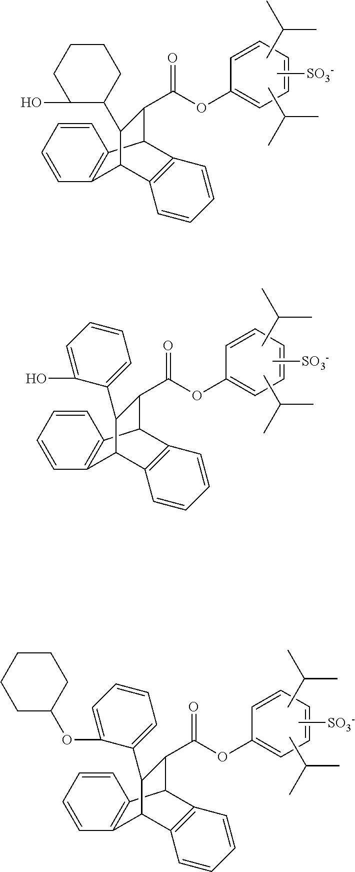

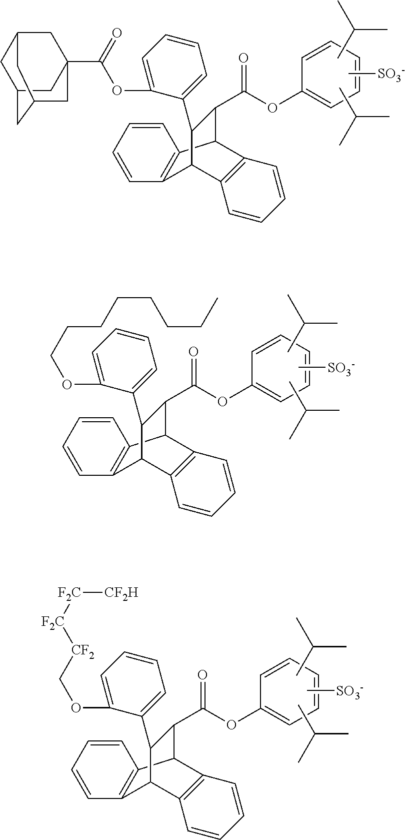

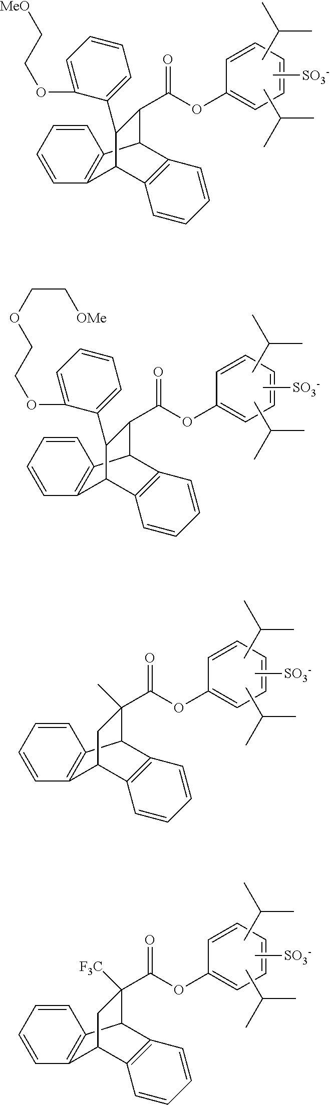

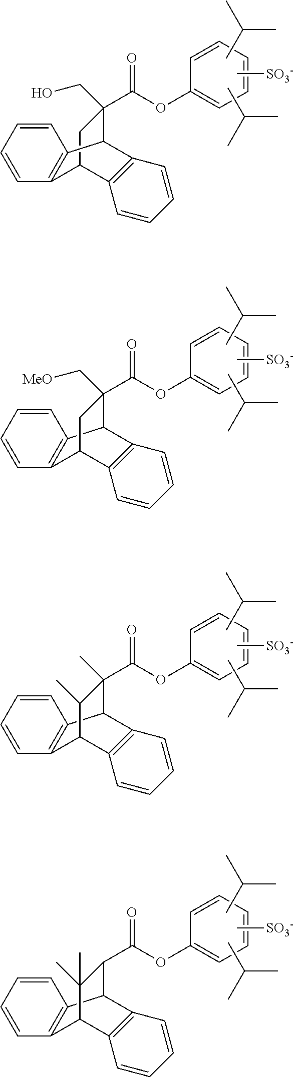

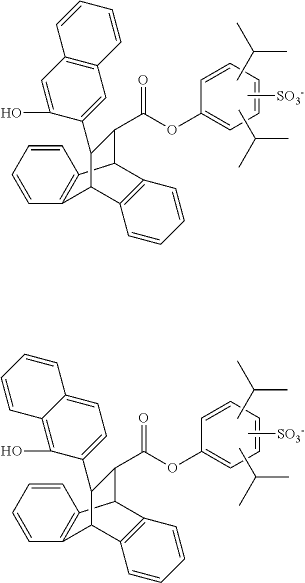

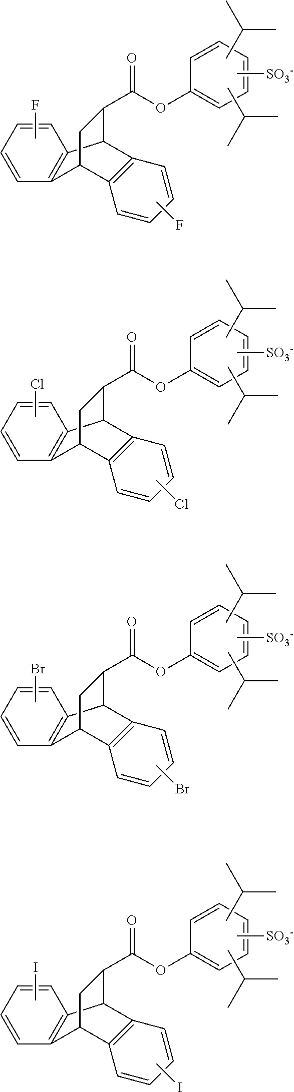

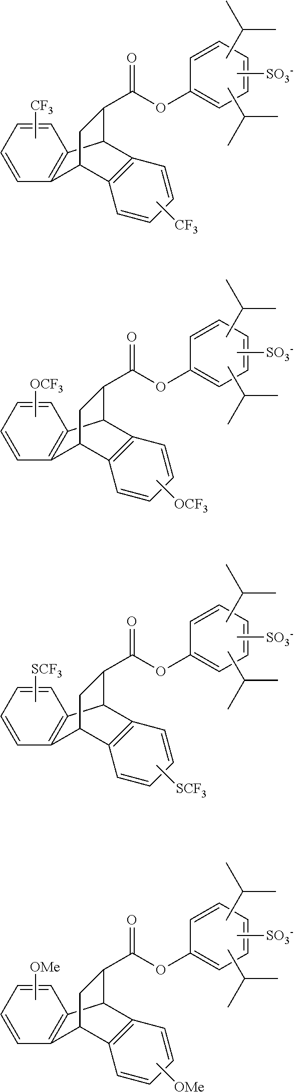

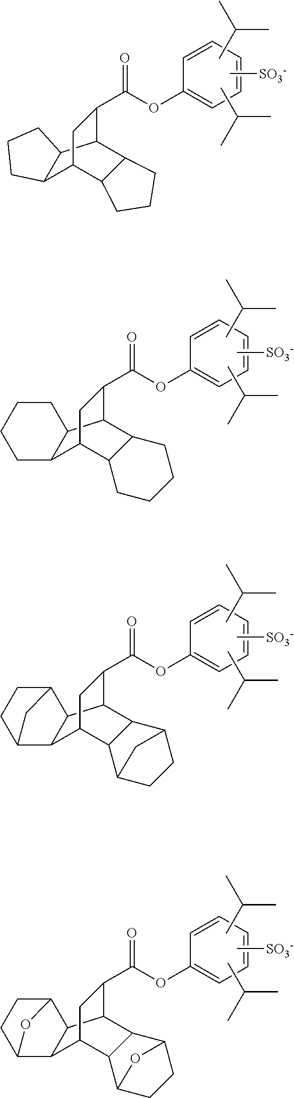

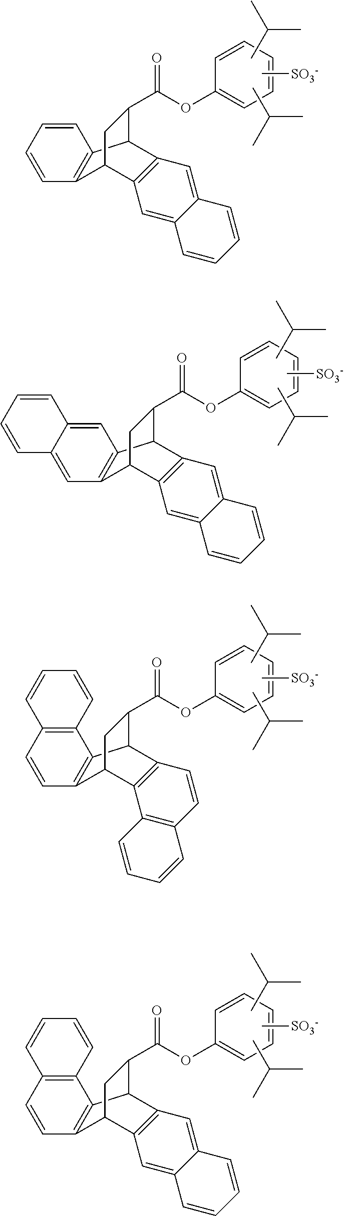

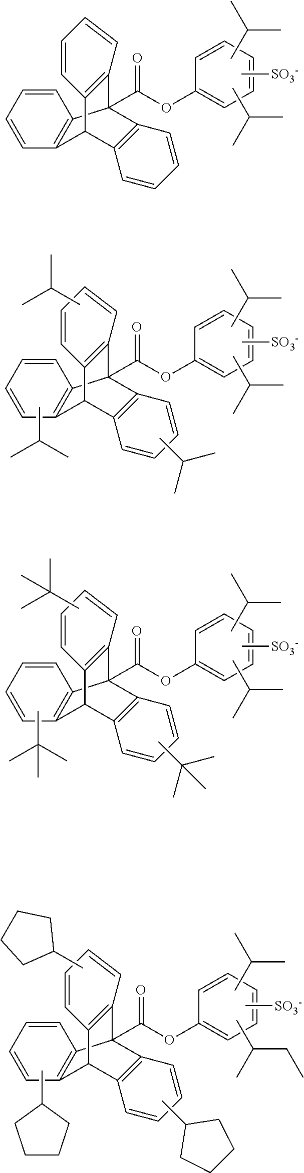

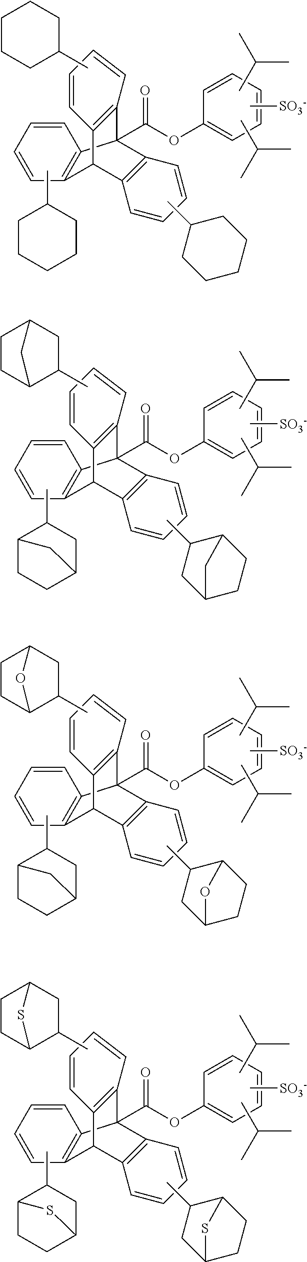

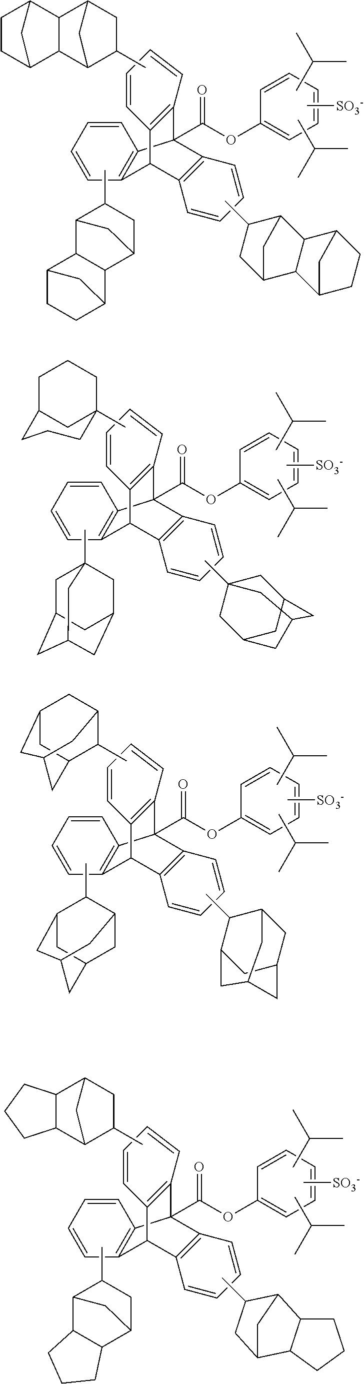

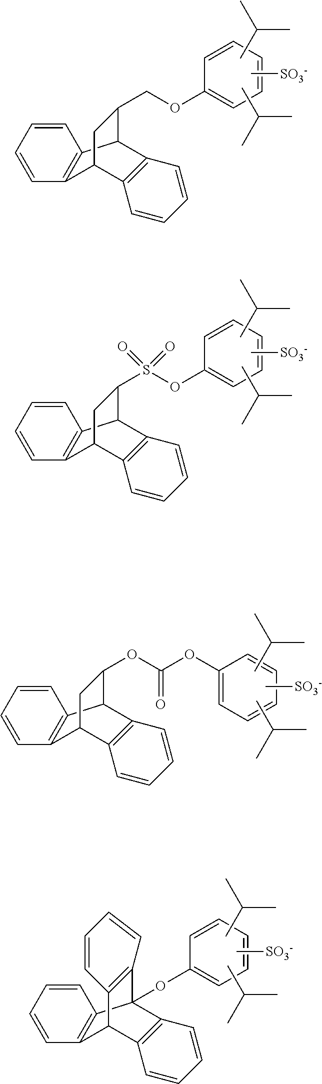

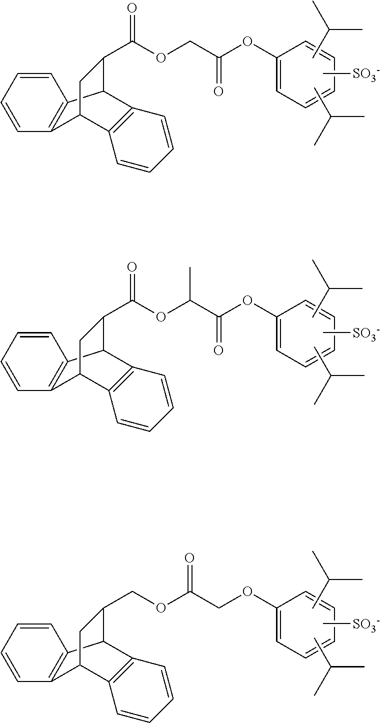

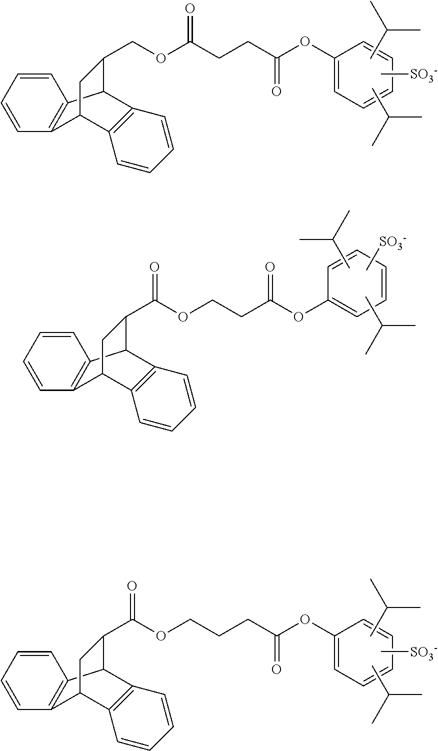

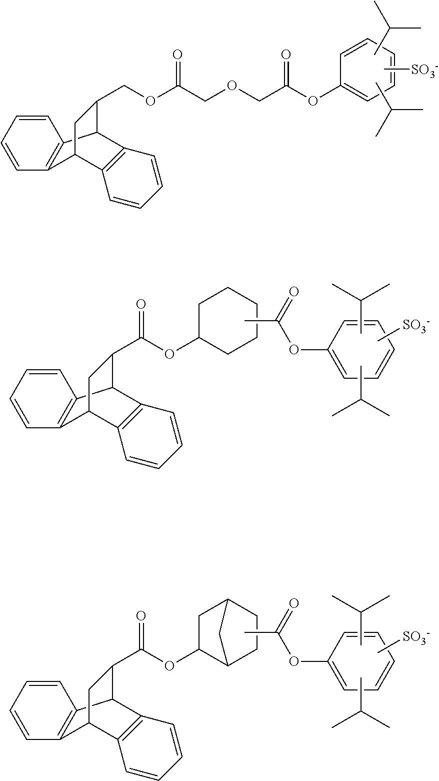

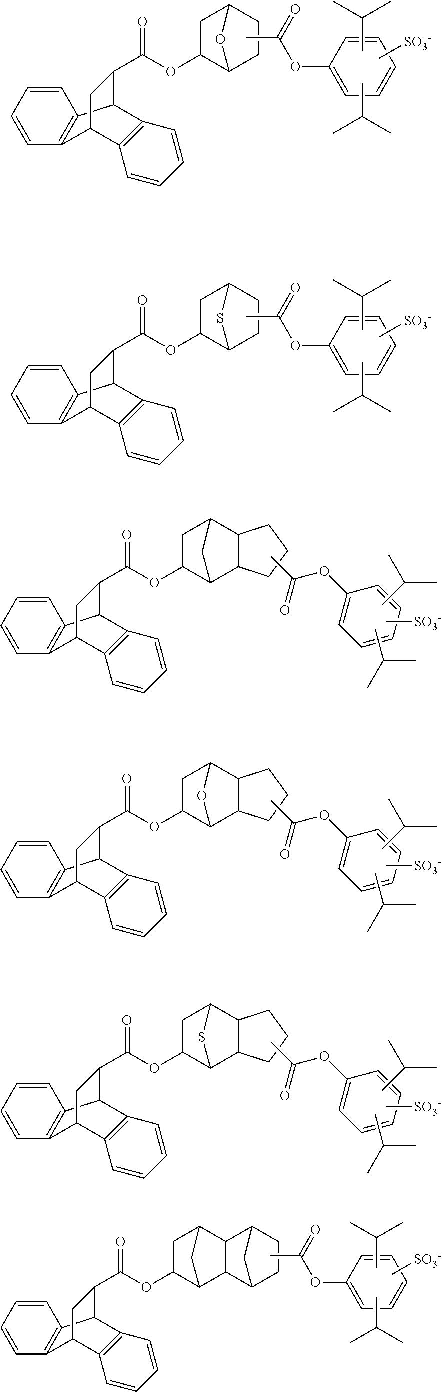

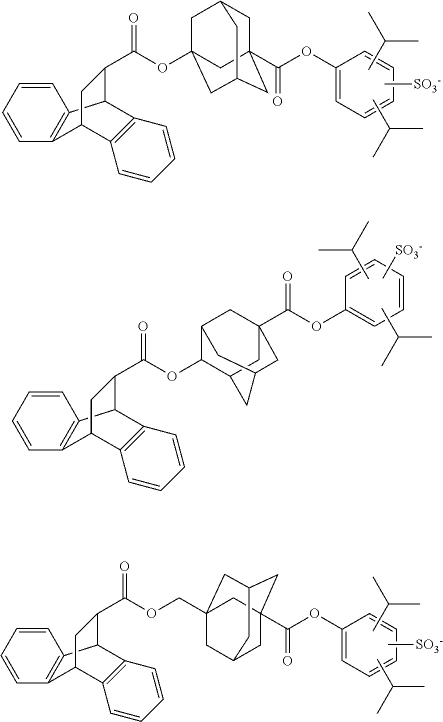

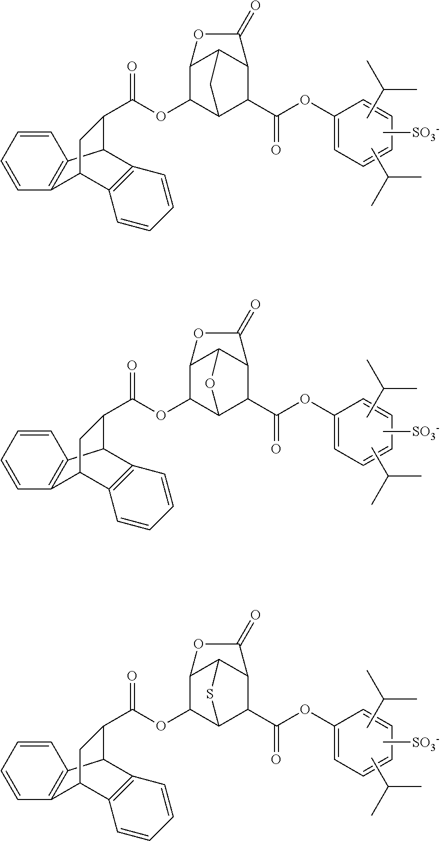

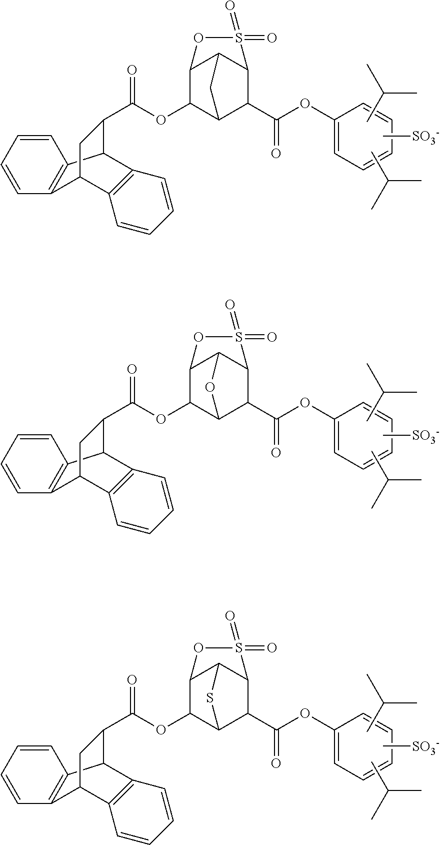

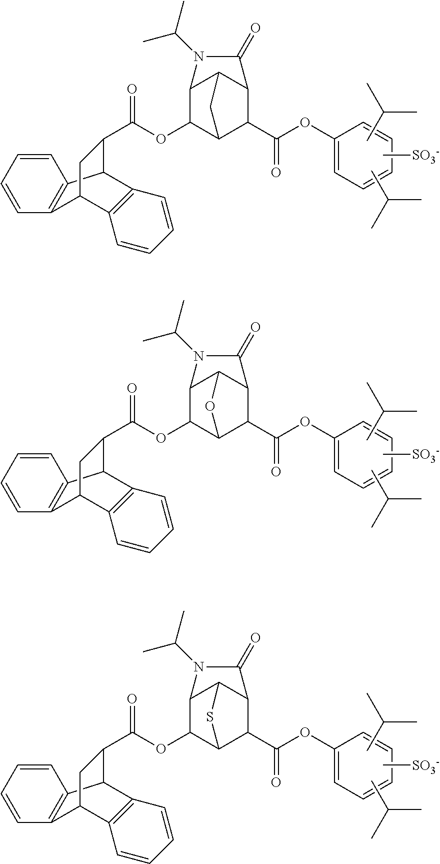

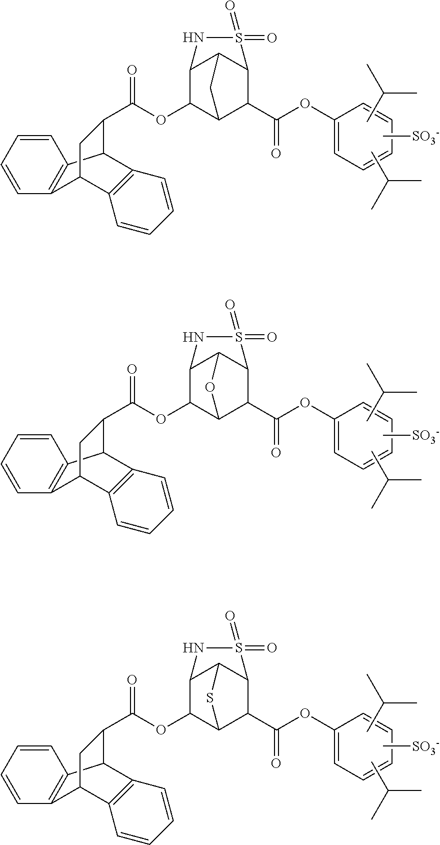

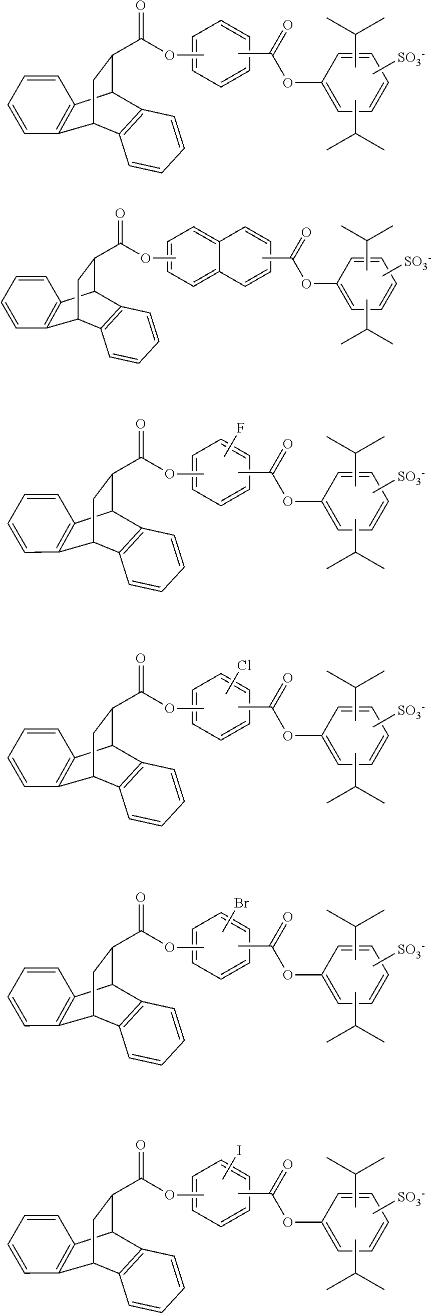

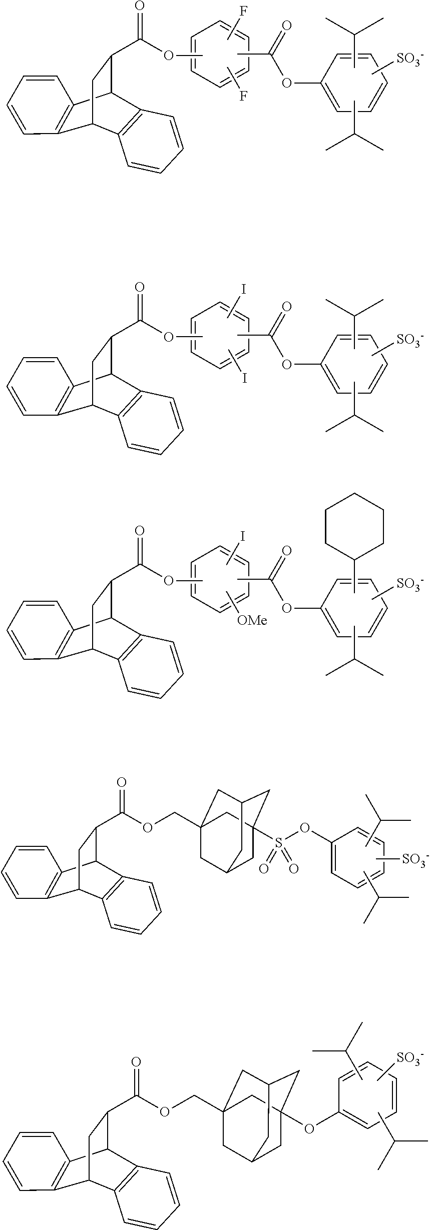

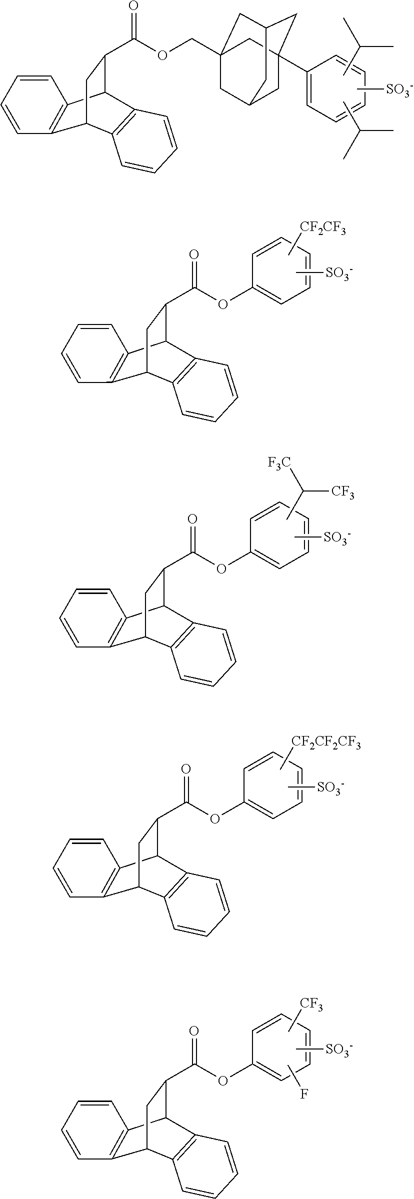

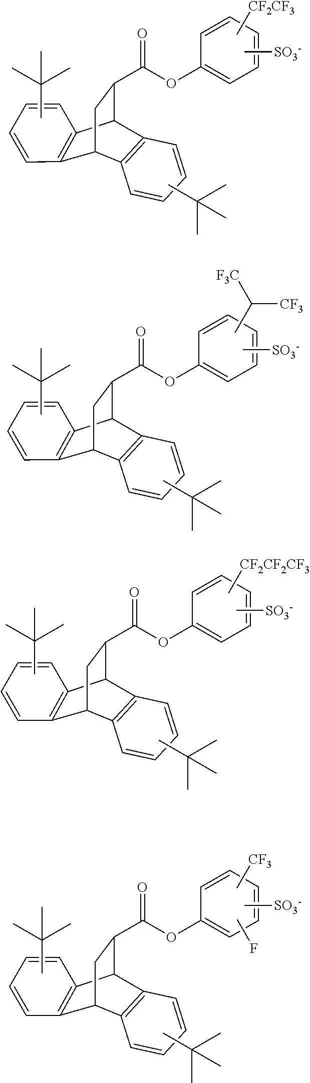

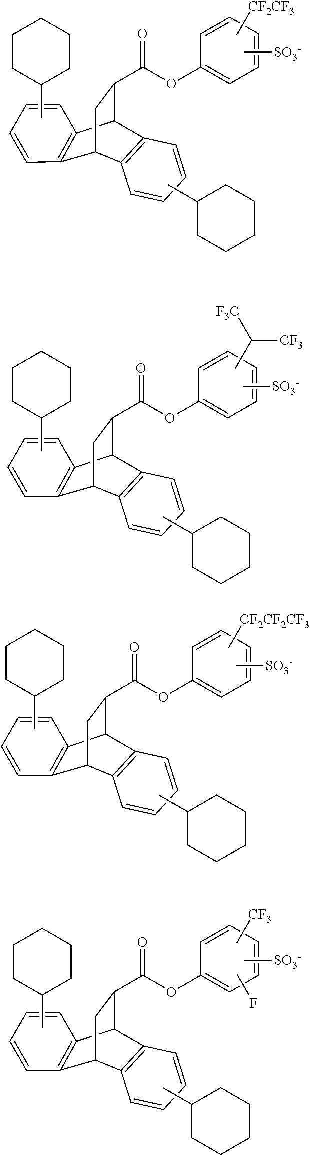

The cation contained in the onium salt of the present invention is represented by the following general formula (1B). Such a cation can be synthesized, for example, by a known method.

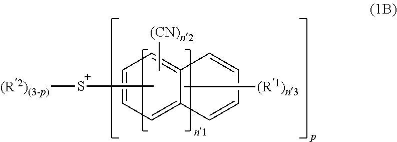

In the formula, “p” represents 1, 2, or 3; “n′1” represents 0 or 1; “n′2” represents 1 or 2; “n′3” represents an integer from 0 to 6; however, when “n′1” represents 0, 1≤n′2+n′3≤5, and when “n′1” represents 1, 1≤n′2+n′3≤7;
R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n′3” represents 2 to 6, each R′¹ may be the same as or different from each other, and two R′¹ may be bonded to each other to form a ring together with the carbon atoms to which they are bonded;
R′² represents a halogen atom or a hydrocarbyl group having 1 to 30 carbon atoms and optionally containing a heteroatom; when “p” represents 1, two R′² may be the same as or different from each other; and two of the three substituents bonded to S⁺ may be bonded to each other to form a ring together with the sulfur atom to which they are bonded.

In the general formula (1B), “p” represents 1, 2 or 3.

In the general formula (1B), “n′1” represents 0 or 1. When “n′1” is 0, the structure is a benzene ring, and when “n′1” is 1, it is a naphthalene ring; however, from the viewpoint of solvent solubility, it is preferable that “n′1” is 0, giving a benzene ring. “n′2” represents 1 or 2. From the viewpoint of raw material procurement, it is preferable that “n′2” is 1. “n′3” represents an integer from 0 to 6. From the viewpoint of raw material procurement, “n′3” is preferably 0, 1, or 2. However, when “n′1” is 0, 1≤n′2+n′3≤5, and when “n′1” is 1, 1≤n′2+n′3≤7.

In the general formula (1B), R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Examples of the halogen atom include a fluorine atom, a chlorine atom, a bromine atom, an iodine atom, etc. The hydrocarbyl groups and the hydrocarbyl moieties of the hydrocarbyloxy groups and the hydrocarbylthio groups may be saturated or unsaturated and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, 1-propenyl, 2-propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl and naphthyl groups; aralkyl groups having 7 to 20 carbon atoms, such as benzyl, 1-phenylethyl, and 2-phenylethyl groups; and groups obtained by combining these, etc. Of these, aryl groups are preferable. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. When “n′3” is 2 to 6, each R′¹ may be the same as or different from each other. Also, when “n′3” is 2 to 6, a plurality of R′¹ may be bonded to each other to form a ring together with the carbon atoms to which they are bonded. The ring is preferably a 5- to 8-member ring.

In the general formula (1B), R′² represents a halogen atom, or a hydrocarbyl group having 1 to 30 carbon atoms and optionally containing a heteroatom. When “p” is 1, two R′² may be the same as or different from each other.

Specific examples of the halogen atom represented by R′² include a fluorine atom, a chlorine atom, a bromine atom, an iodine atom, etc.

The hydrocarbyl group represented by R′² may be saturated or unsaturated, and may be linear, branched, or cyclic. Specific examples thereof include alkyl groups having 1 to 30 carbon atoms, such as methyl, ethyl, n-propyl, isopropyl, n-butyl, isobutyl, sec-butyl, tert-butyl groups; cyclic saturated hydrocarbyl groups having 3 to 30 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 30 carbon atoms, such as vinyl, 1-propenyl, 2-propenyl, butenyl, and hexenyl groups; cyclic unsaturated hydrocarbyl groups having 3 to 30 carbon atoms, such as cyclohexenyl groups; aryl groups having 6 to 30 carbon atoms, such as phenyl, naphthyl, and thienyl groups; aralkyl groups having 7 to 30 carbon atoms, such as benzyl, 1-phenylethyl, and 2-phenylethyl groups; and groups obtained by combining these, etc.; however, an aryl group is preferable. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Also, two of the three substituents bonded to S⁺ may be bonded to each other to form a ring with the sulfur atom to which they are bonded. In this case, specific examples of the ring structure include those represented by the following formulae, etc.

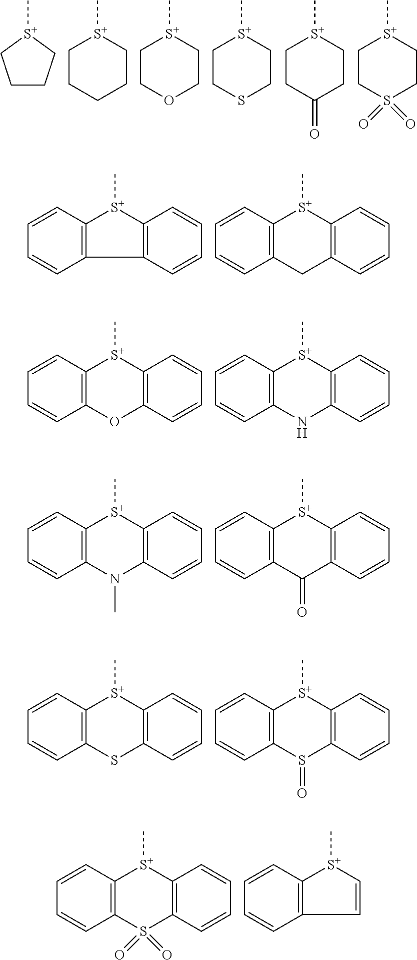

In the formulae, a broken line represents an attachment point.

The cation represented by the general formula (1B) is preferably represented by the following general formula (1B-1).

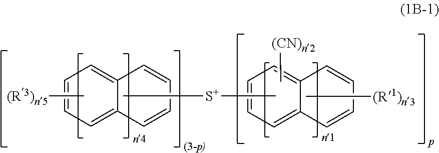

In the formula, “p”, “n′1” to “n′3”, and R′¹ are as defined above;
“n′4” represents 0 or 1; “n′5” represents an integer from 0 to 5;
R′³ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and when “n′5” represents 2 to 5, each R′³ may be the same as or different from each other, and two R′³ may be bonded to each other to form a ring together with the carbon atoms to which they are bonded.

In the general formula (1B-1), “n′4” represents 0 or 1. When “n′4” is 0, the structure is a benzene ring, and when “n′4” is 1, it is a naphthalene ring; however, from the viewpoint of solvent solubility, it is preferable that “n′4” is 0, giving a benzene ring. “n′5” represents an integer from 0 to 5. From the viewpoint of raw material procurement, “n′5” is preferably 0, 1, or 2.

In the general formula (1B-1), R′³ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl groups and the hydrocarbyl moieties of the hydrocarbyloxy groups and the hydrocarbylthio groups may be saturated or unsaturated and may be linear, branched, or cyclic. Specific examples thereof include the same ones as those exemplified as the hydrocarbyl group represented by R′¹, but are not limited to these. When “n′3” is 2 to 5, each R′³ may be the same as or different from each other, and two R′³ may be bonded to each other to form a ring together with the carbon atoms to which they are bonded.

Specific examples of the cation represented by the general formula (1B) include, but are not limited to, the following. Note that in the following formulae, Me represents a methyl group.

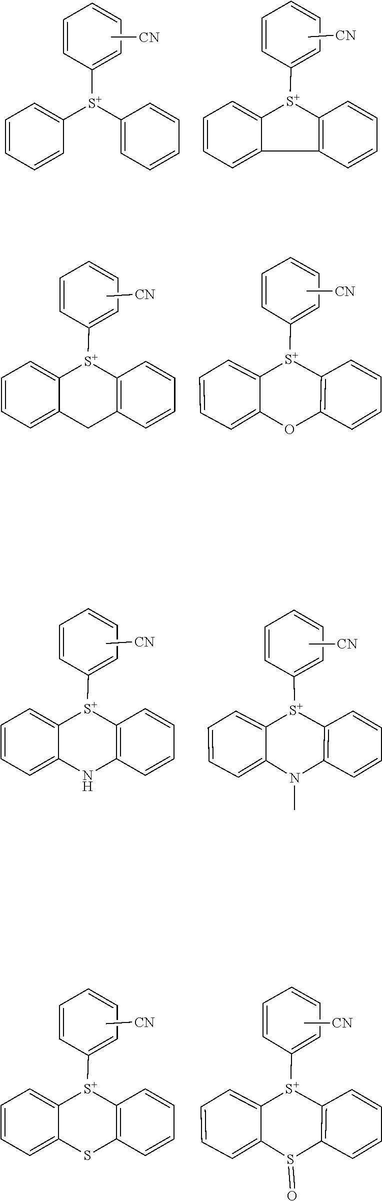

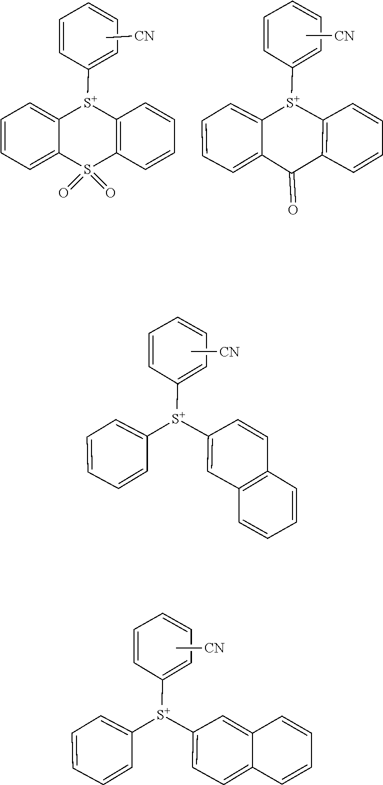

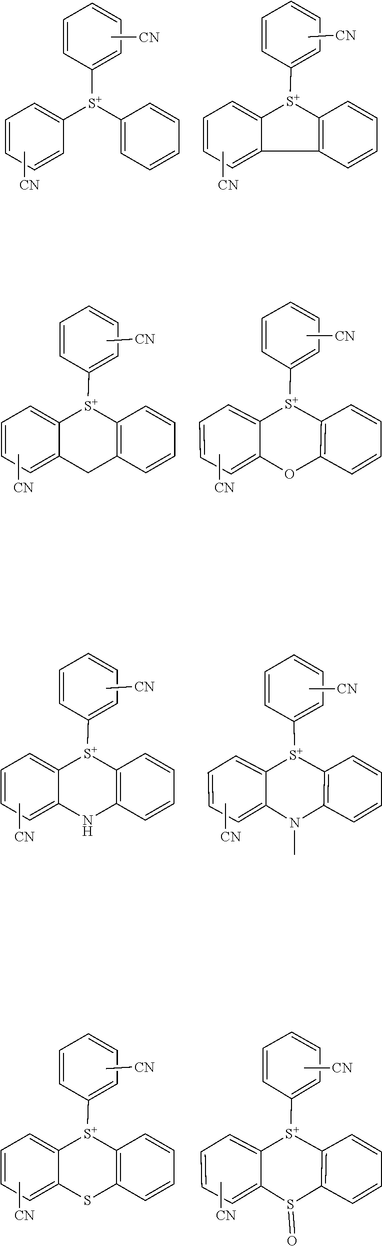

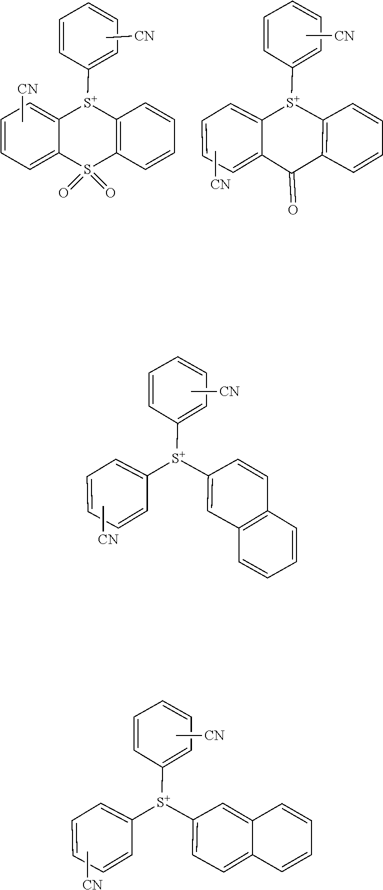

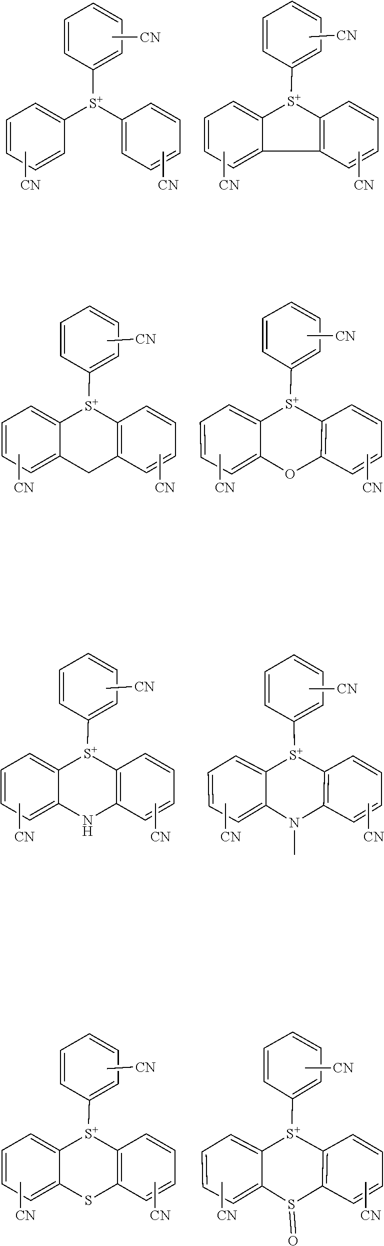

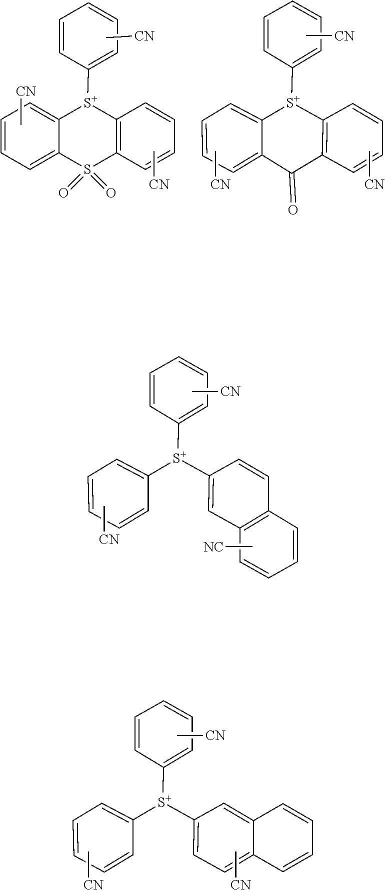

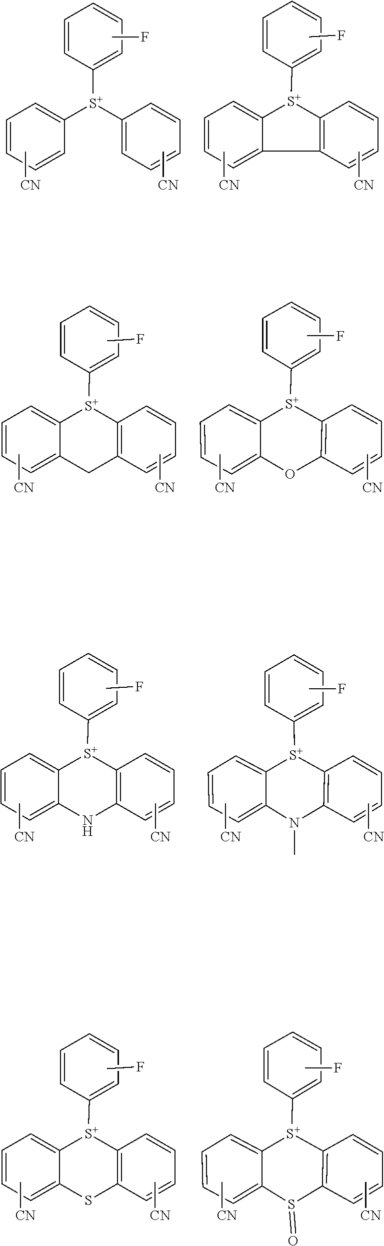

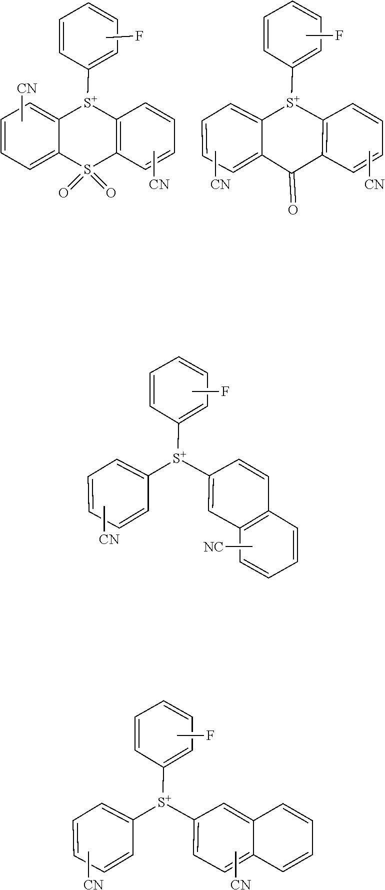

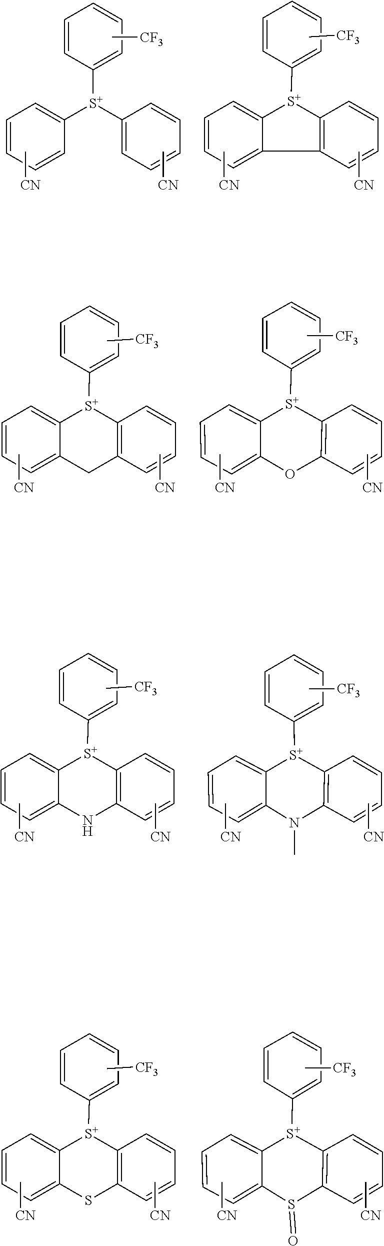

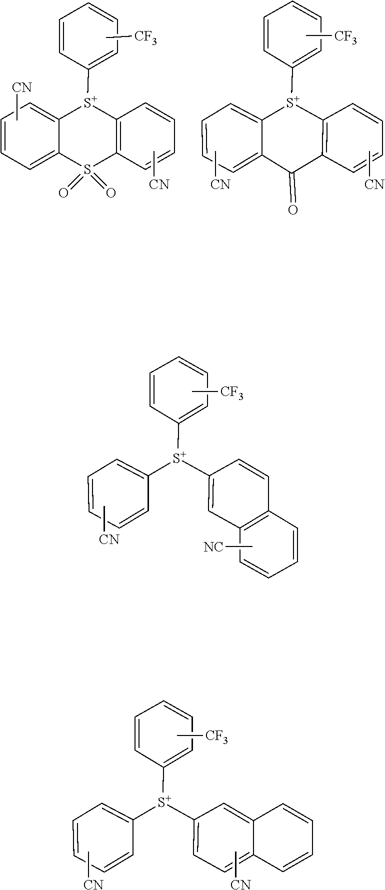

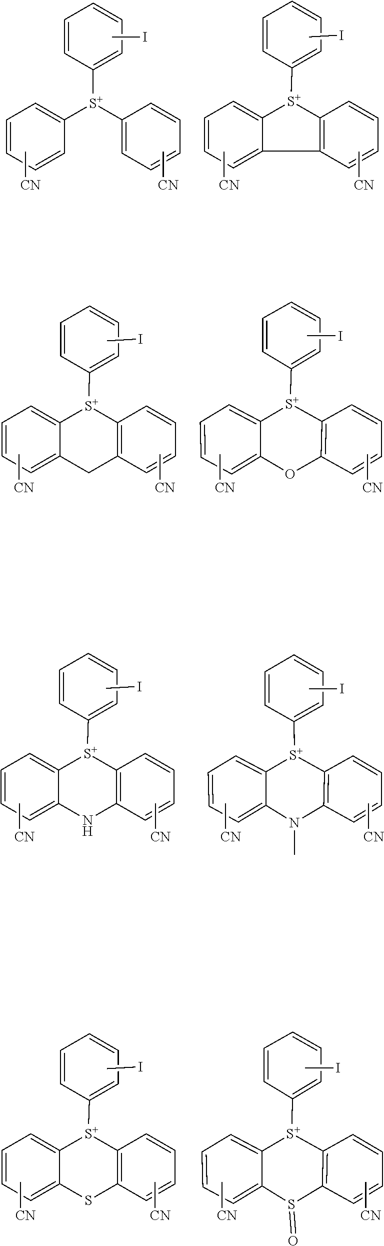

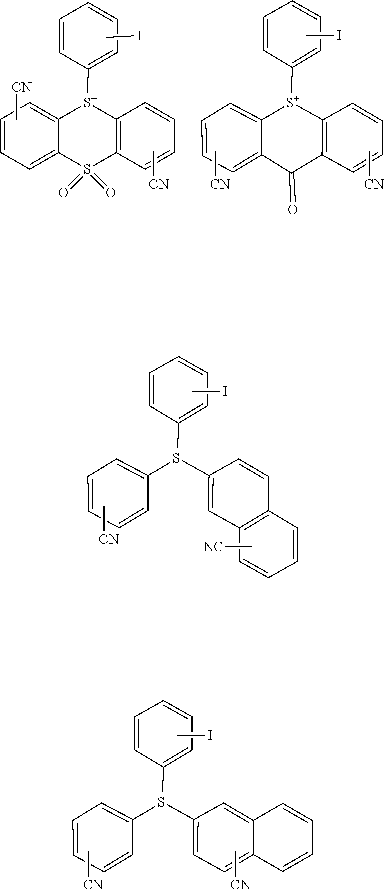

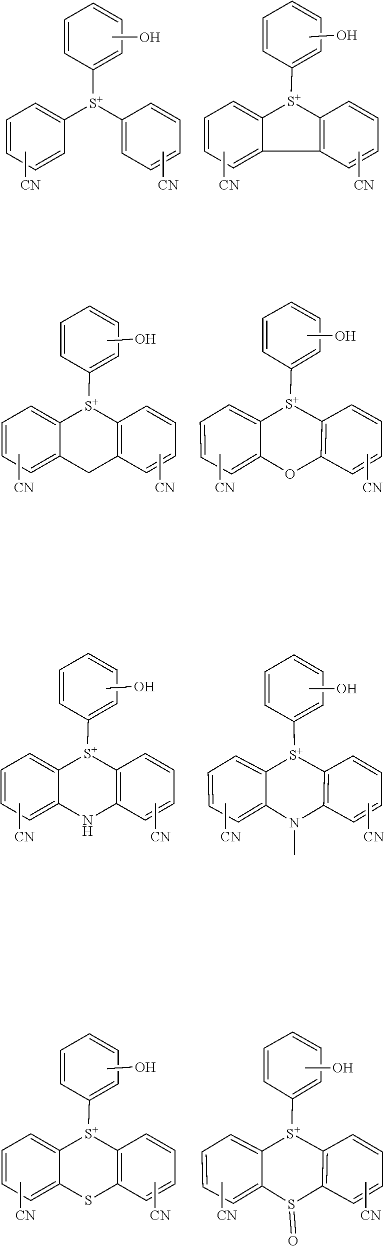

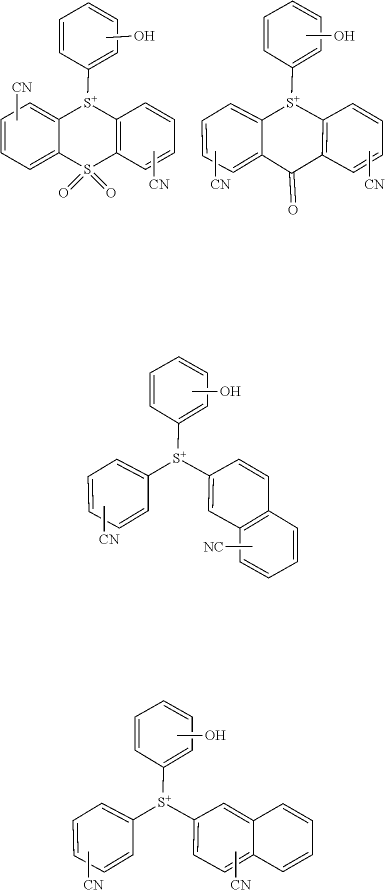

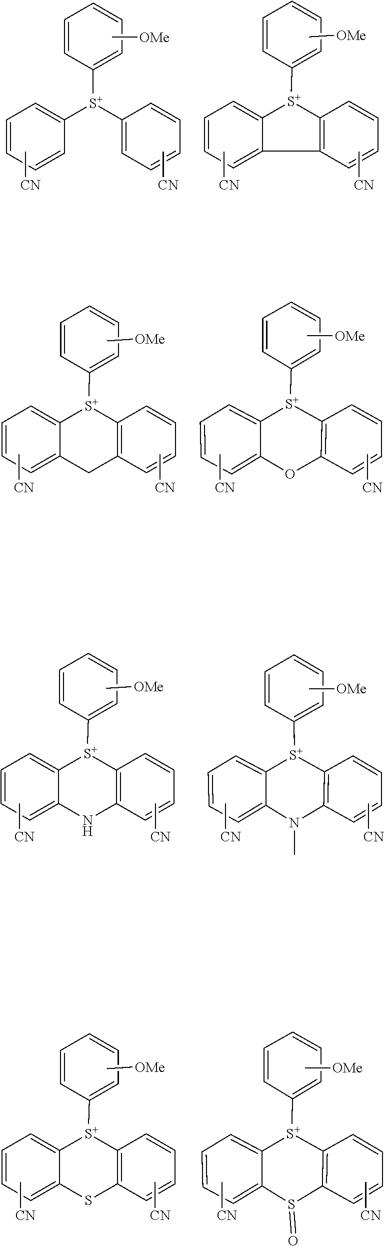

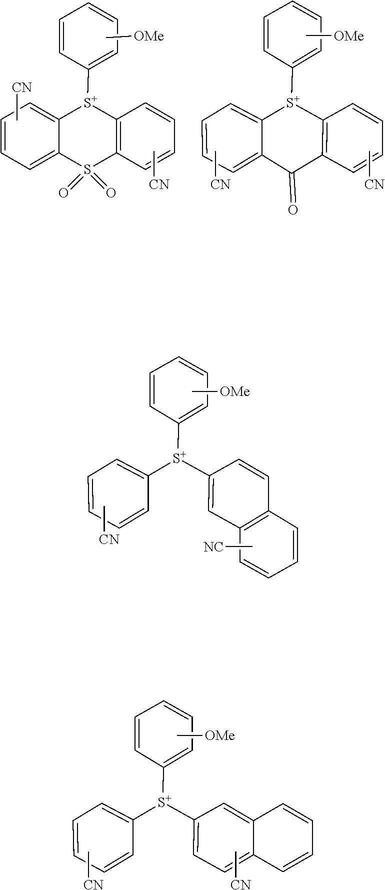

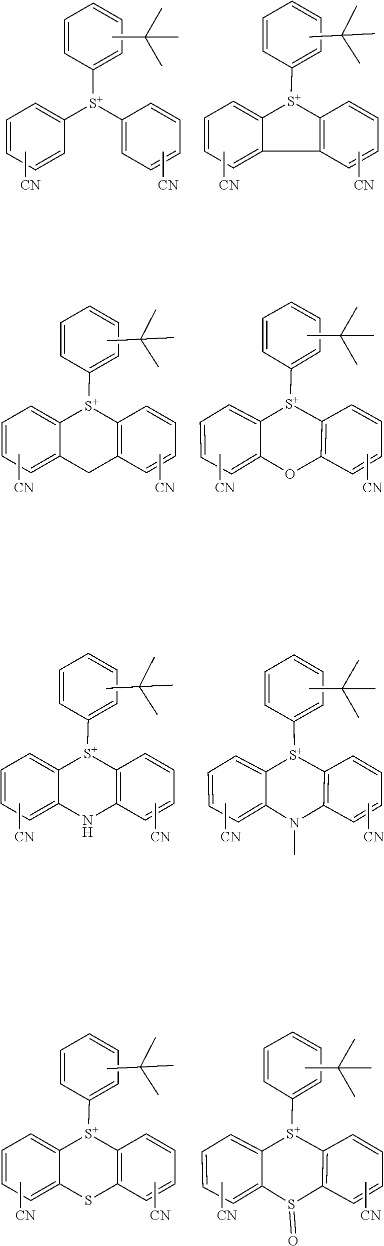

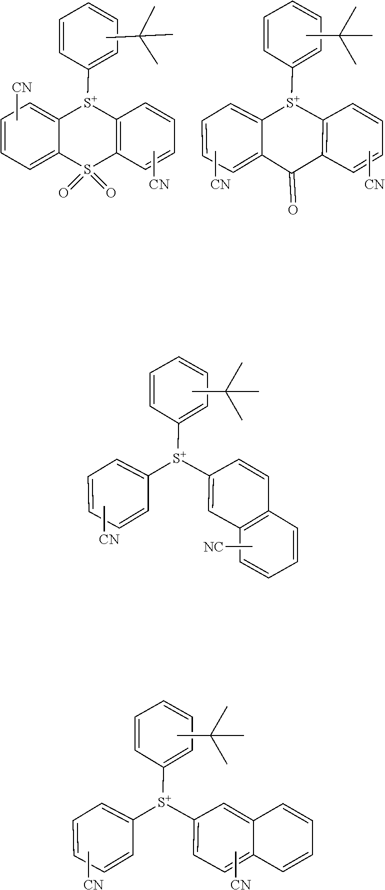

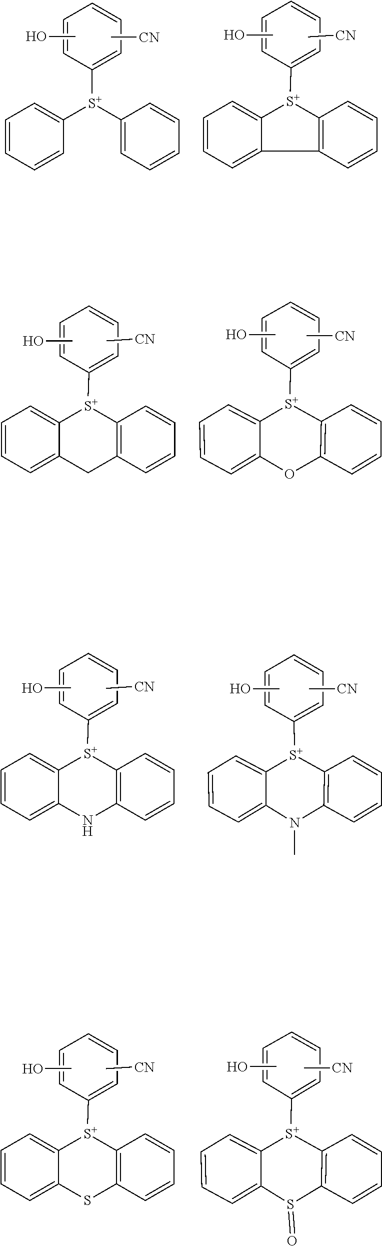

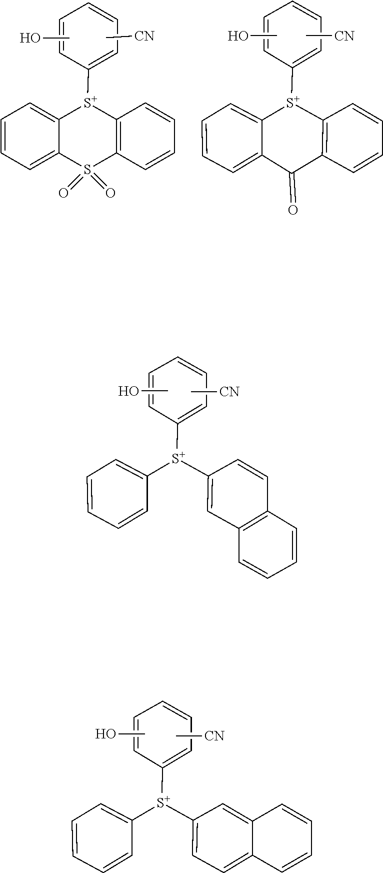

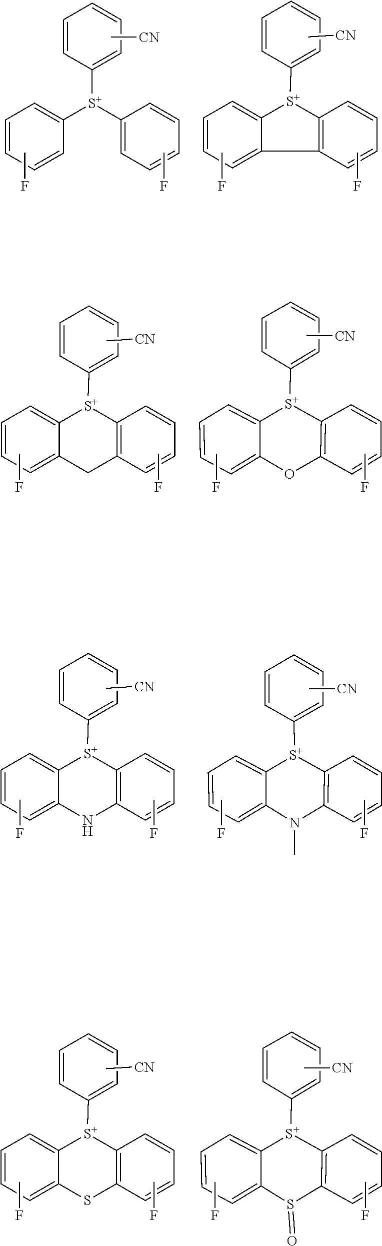

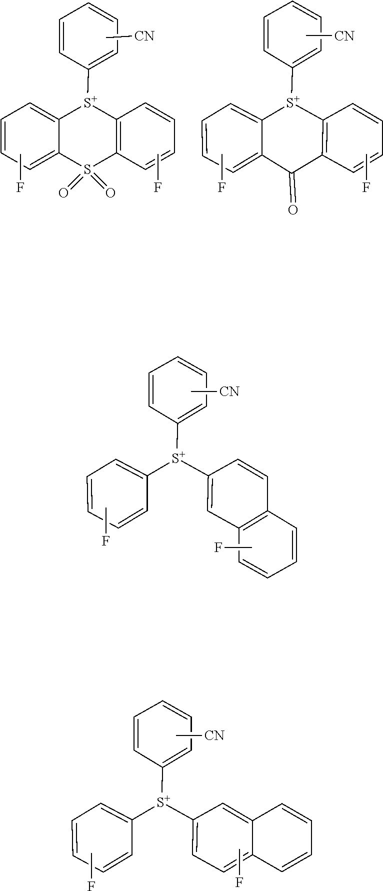

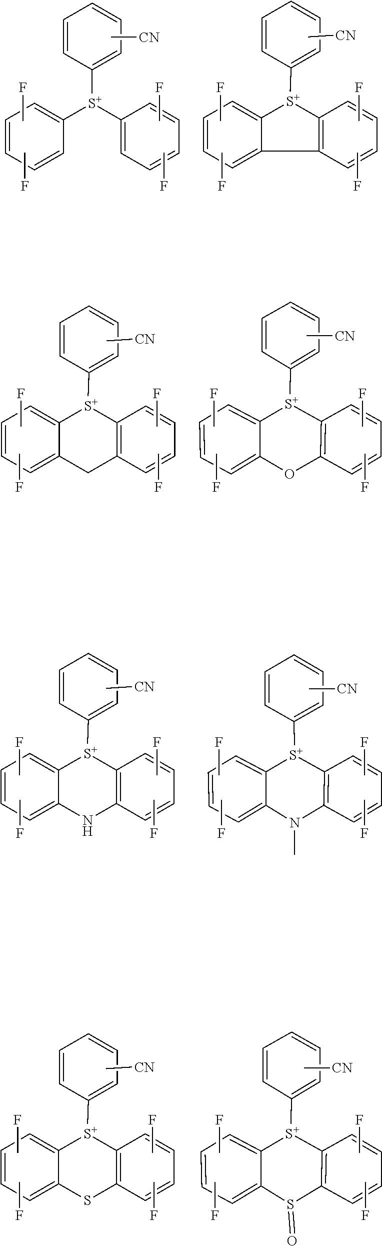

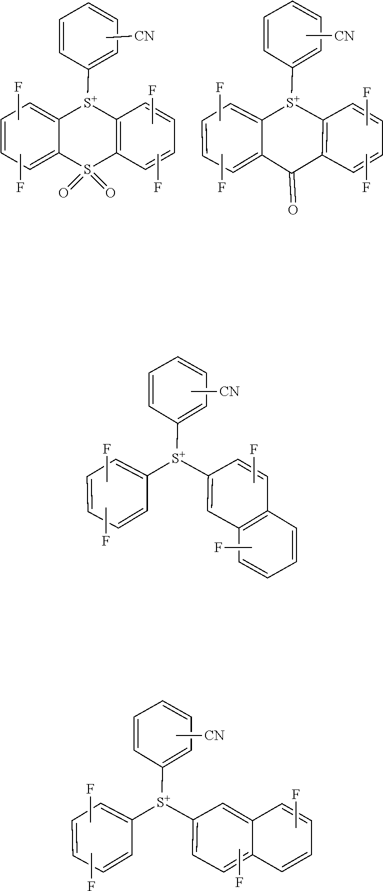

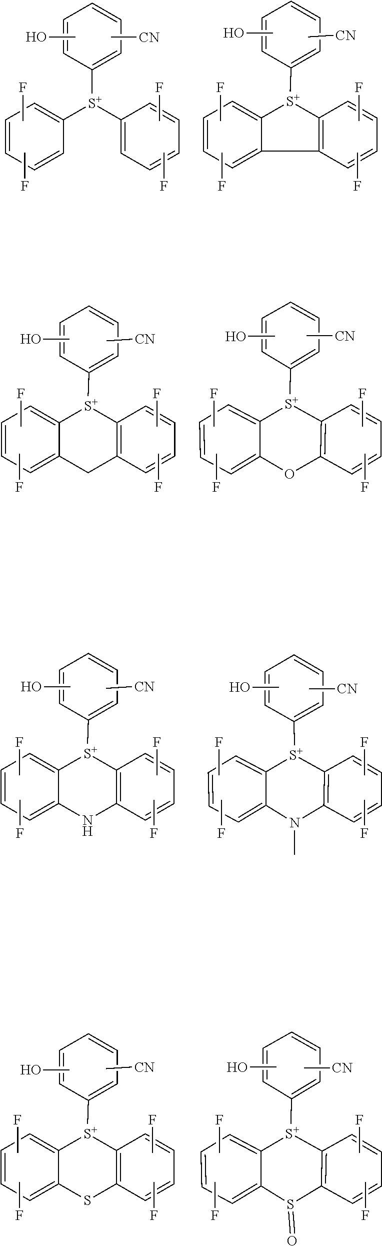

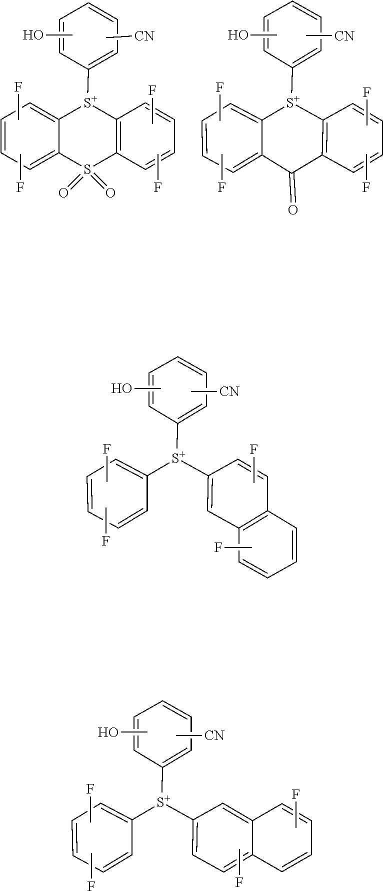

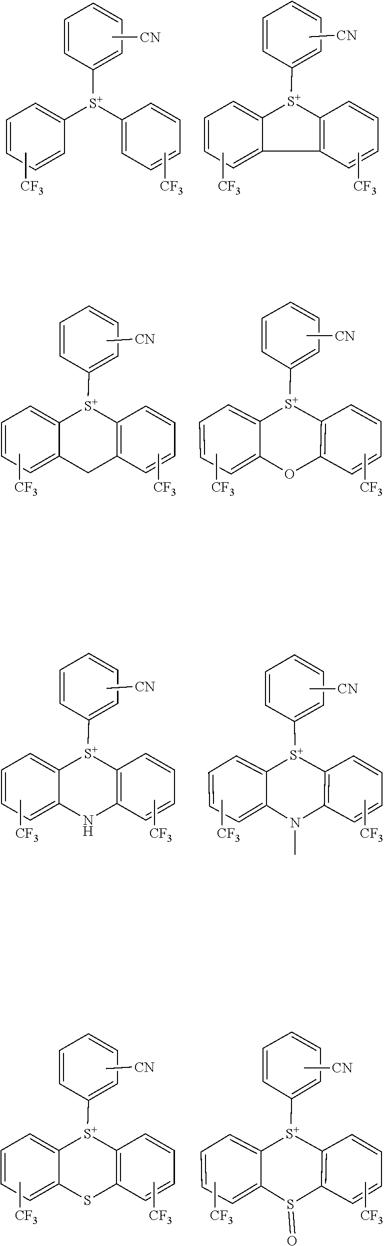

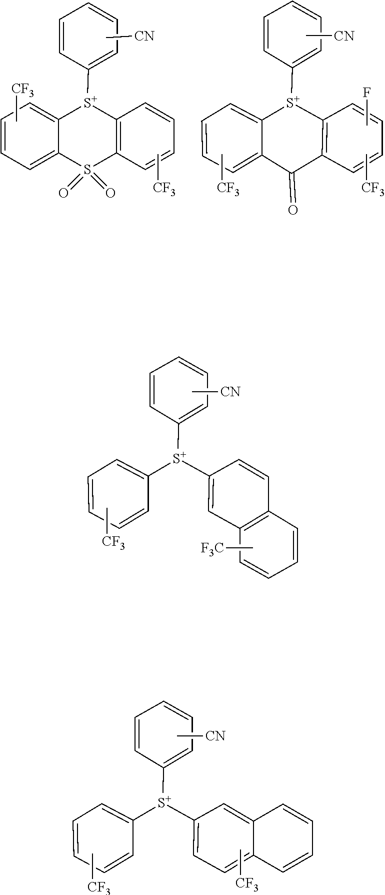

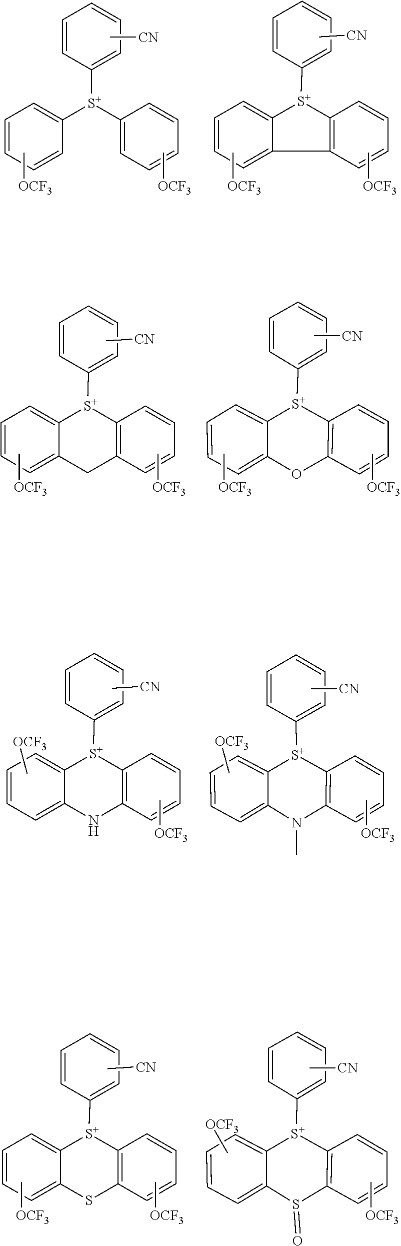

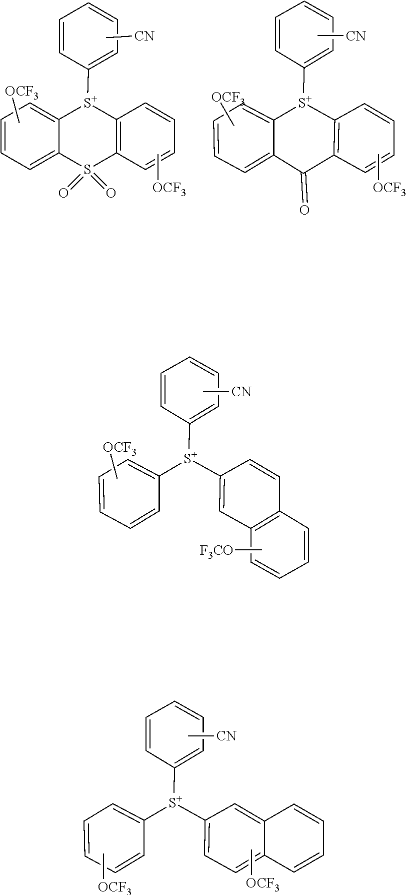

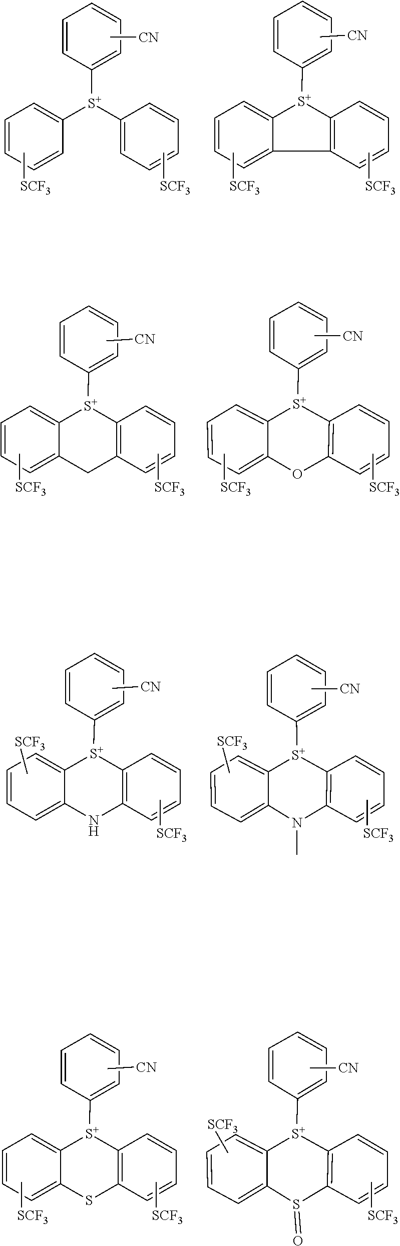

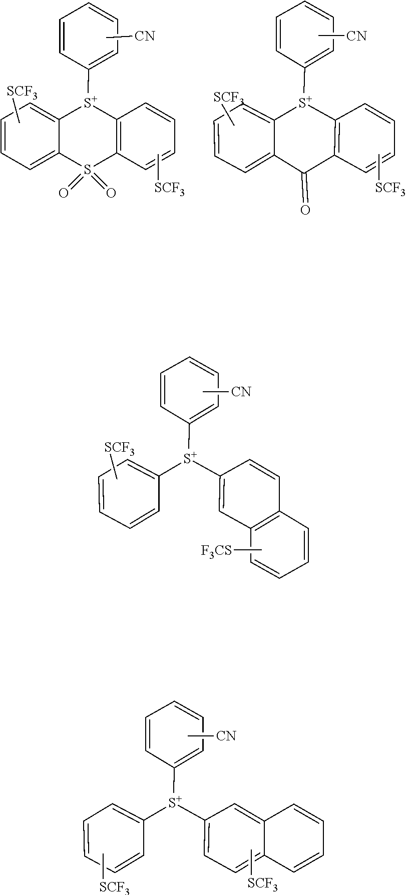

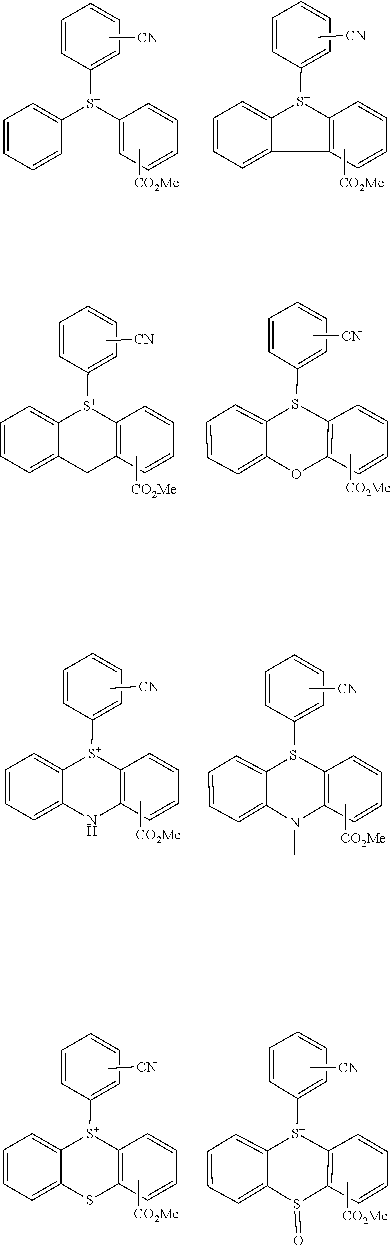

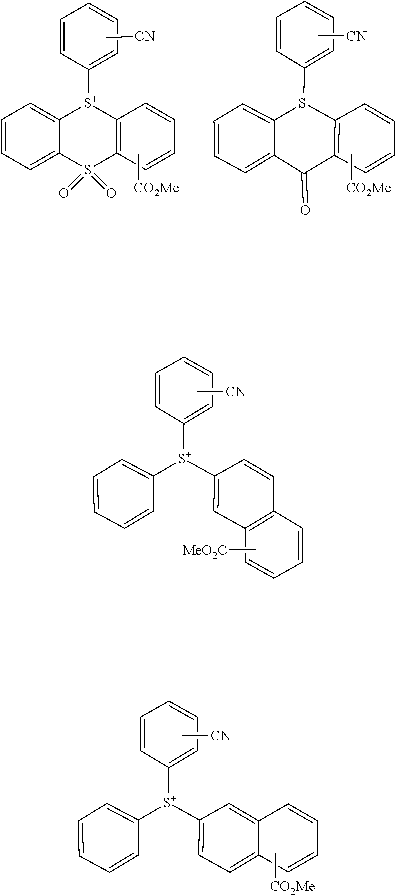

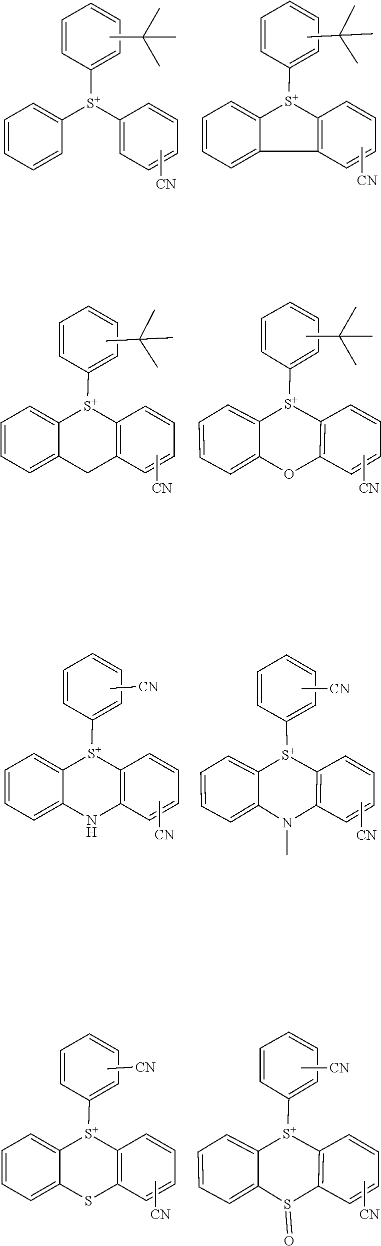

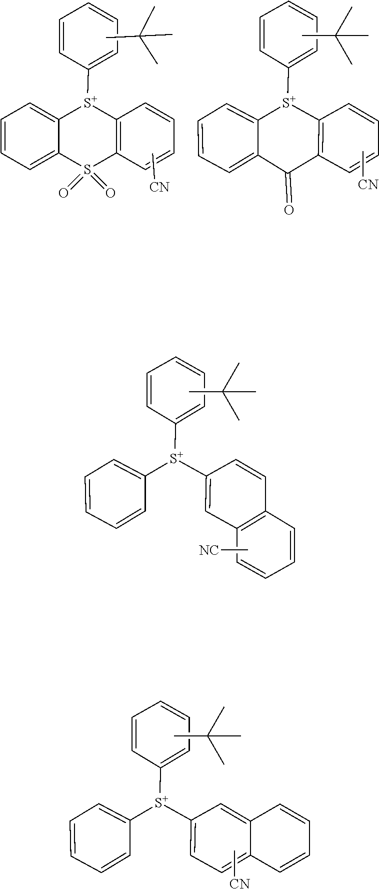

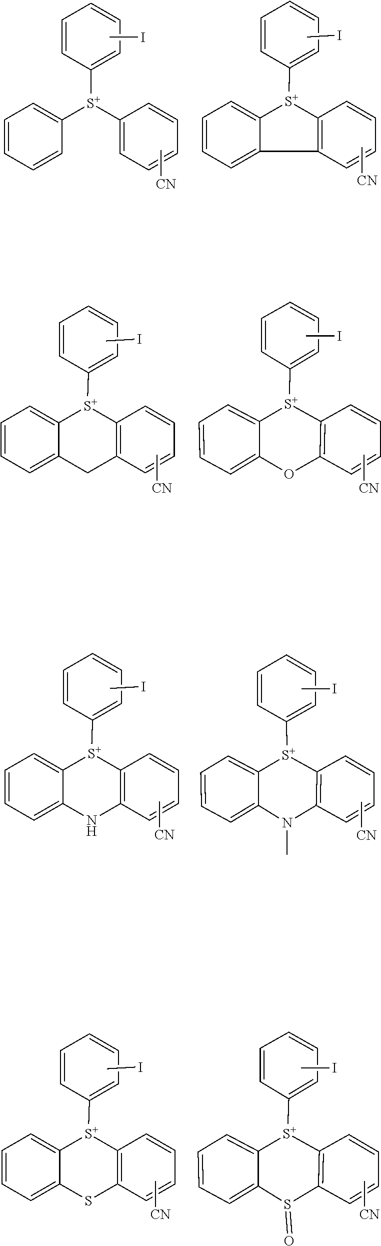

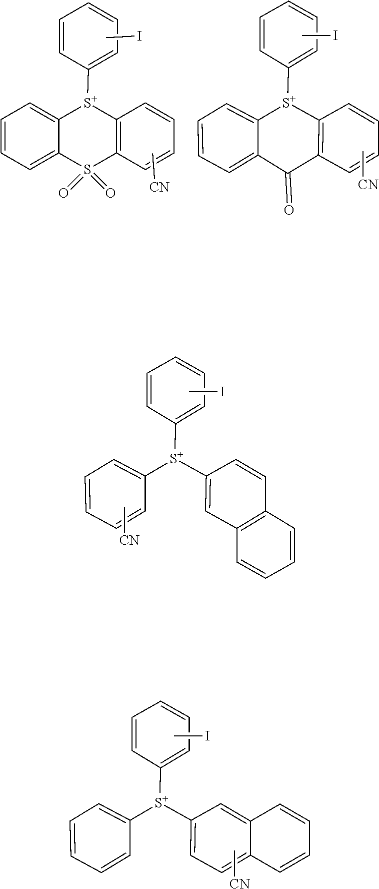

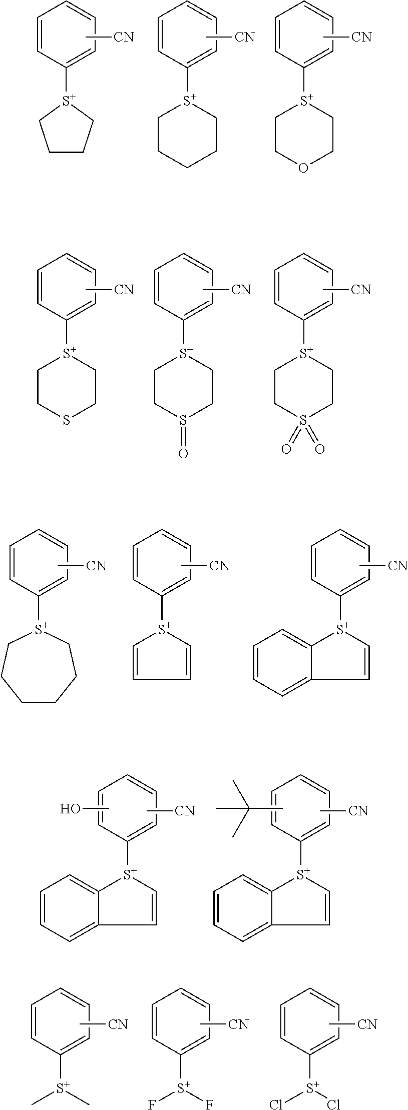

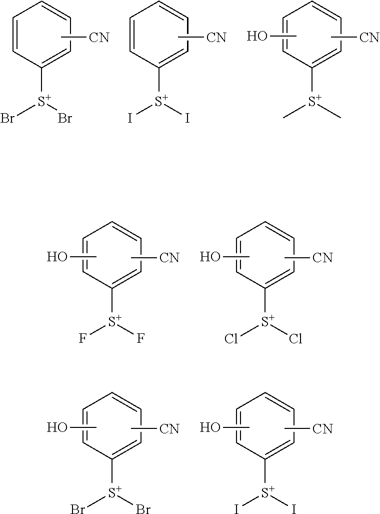

Specific examples of the onium salt of the present invention include any combination of the above-mentioned anions and cations.

Methods for synthesizing the onium salt of the present invention include methods described in JP2010-155824A, JP7067271B2, etc.; however, the above-mentioned production methods are merely examples, and the production method of the onium salt of the present invention is not limited to these.

The characteristic structural feature of the onium salt of the present invention is that it has an aromatic sulfonate anion substituted with a hydrocarbyl group containing at least one aromatic ring, and a sulfonium cation containing a cyano group. It is known that secondary electrons are emitted when the base polymer is irradiated with EUV light, and the cyano group of the sulfonium cation lowers the energy level of the lowest unoccupied molecular orbital (LUMO) in frontier orbital theory due to its electron-withdrawing effect, making it easier to receive the generated secondary electrons, promoting the decomposition of the cation, and effectively generating acid. Furthermore, since the nitrogen atom of the cyano group has a lone pair of electrons, it is expected to function as an acid diffusion suppressing group by interacting with the protons of the generated acid. On the other hand, the hydrocarbyl group containing at least one aromatic ring has a large excluded volume and acts as a bulky substituent, highly suppressing the diffusion of the generated acid. In particular, such effects are easily obtained when the hydrocarbyl group containing at least one aromatic ring is an aromatic ring structure having a substituent represented by the general formula (W-1) or a condensed ring structure having a substituent represented by the general formula (W-2). In addition, its resistance to alkaline developers reduces film loss in the unexposed areas of the pattern. On the other hand, the aromatic sulfonate structure generates a rigid acid structure, which has the effect of suppressing acid diffusion. The aromatic ring forming the aromatic sulfonate structure preferably contains a fluorine atom or a sulfonate ester bond having electron-withdrawing properties as a linking group, which increases the acidity of the generated acid and enables the acid-labile groups of the base polymer to be efficiently deprotected. In addition, fluorine atoms are an element that has a high absorption effect on EUV light, although not as effectively as iodine atoms; therefore, an increase in the number of fluorine atoms increases the amount of secondary electrons generated, promoting the decomposition of cations and contributing to higher sensitivity. JP7109178B2 describes an alkanesulfonic acid type photoacid generator having 2 to 4 fluorine atoms, but because it is an alkanesulfonic acid, acid diffusion is relatively large and solvent solubility is poor, raising concerns about development defects. Due to these synergistic effects, the resist composition containing the onium salt of the present invention has high sensitivity and low acid diffusibility, which enables the formation of patterns that are excellent in terms of LWR of line patterns and CDU of hole patterns and are resistant to pattern collapse, making it ideal for forming fine patterns.

### [Photoacid Generator]

The present invention also provides a photoacid generator consisting of the onium salt described above. That is, the onium salt can be suitably used as a photoacid generator.

### [Chemically Amplified Resist Composition]

The present invention also provides a chemically amplified resist composition, which contains the above-described photoacid generator (A).

### [Photoacid Generator (A)]

The chemically amplified resist composition of the present invention contains as an essential component a photoacid generator consisting of the above-mentioned onium salt, as the photoacid generator (A).

In the chemically amplified resist composition of the present invention, the content of component (A), the photoacid generator consisting of the onium salt, is preferably 0.1 to 40 parts by mass, and more preferably 0.5 to 30 parts by mass, per 80 parts by mass of the base polymer (B) described below. When the content of component (A) is within the above range, the sensitivity and resolution are good, and there is no risk of problems with foreign matter occurring after development of or during stripping of the resist film. This is therefore preferable. The photoacid generator (A) may be used alone or in a combination of two or more types.

### [Base Polymer (B)]

The chemically amplified resist composition of the present invention may further contain a base polymer as component (B). The base polymer (B) contains a repeating unit represented by the following general formula (a1) (hereinafter also referred to as “repeating unit-a1”) and/or a repeating unit represented by the following general formula (a2) (hereinafter also referred to as “repeating unit-a2”). That is, it is preferable that the chemically amplified resist composition further contains a base polymer containing a repeating unit represented by either one or both of the following general formulae (a1) and (a2).

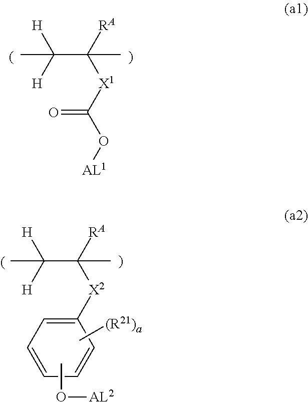

In the formulae, R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
* represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a” represents an integer from 0 to 4.

In the general formulae (a1) and (a2), R^{A} each independently represents a hydrogen atom, a fluorine atom, a methyl group, or a trifluoromethyl group.

In the general formula (a1), X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom. X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring; and * represents an attachment point to a main chain carbon atom.

In the general formula (a2), X² represents a single bond or *—C(═O)—O—. * represents an attachment point to a main chain carbon atom. R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “a” represents an integer from 0 to 4, and is preferably 0 or 1.

In the general formulae (a1) and (a2), AL¹ and AL² each independently represent an acid-labile group. Specific examples of the acid-labile group include those described in JP2013-080033A or JP2013-083821A.

Typical specific examples of the acid-labile group include those represented by the following general formulae (AL-1) to (AL-3).

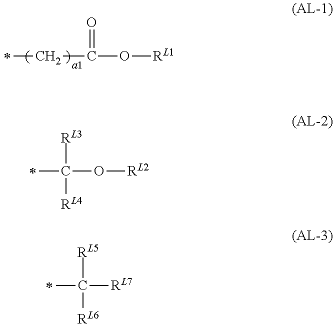

In the formulae, a broken line represents an attachment point.

In the general formulae (AL-1) and (AL-2), R^{L1} and R^{L2} each independently represent a hydrocarbyl group having 1 to 40 carbon atoms and optionally containing a heteroatom such as an oxygen atom, a sulfur atom, a nitrogen atom, or a fluorine atom. The hydrocarbyl group may be saturated or unsaturated and may be linear, branched, or cyclic. The hydrocarbyl group preferably has 1 to 20 carbon atoms.

In the general formula (AL-1), “a1” represents an integer from 0 to 10, and preferably an integer from 1 to 5.

In the general formula (AL-2), R^{L3} and R^{L4} each independently represent a hydrogen atom or a hydrocarbyl group having 1 to 20 carbon atoms and optionally containing a heteroatom such as an oxygen atom, a sulfur atom, a nitrogen atom, or a fluorine atom. The hydrocarbyl group may be saturated or unsaturated and may be linear, branched, or cyclic. The hydrocarbyl group preferably has 1 to 20 carbon atoms. Also, any two of R^{L2}, R^{L3}, and R^{L4} may be bonded to each other to form a ring having 3 to 20 carbon atoms together with the carbon atom, or the carbon atom and oxygen atom, to which they are bonded. The ring is preferably a ring having 4 to 16 carbon atoms, and particularly preferably an alicyclic ring.

In the general formula (AL-3), R^{L5}, R^{L6}, and R^{L7} each independently represent a hydrocarbyl group having 1 to 20 carbon atoms, and optionally containing a heteroatom such as an oxygen atom, a sulfur atom, a nitrogen atom, or a fluorine atom. The hydrocarbyl group may be saturated or unsaturated and may be linear, branched, or cyclic. The hydrocarbyl group preferably has 1 to 20 carbon atoms. Also, any two of R^{L5}, R^{L6}, and R^{L7} may be bonded to each other to form a ring having 3 to 20 carbon atoms together with the carbon atom to which they are bonded. The ring is preferably a ring having 4 to 16 carbon atoms, and particularly preferably an alicyclic ring.

Specific examples of the repeating unit-a1 include, but are not limited to, the following. Note that in the following formulae, R^{A} and AL¹ are as defined above.

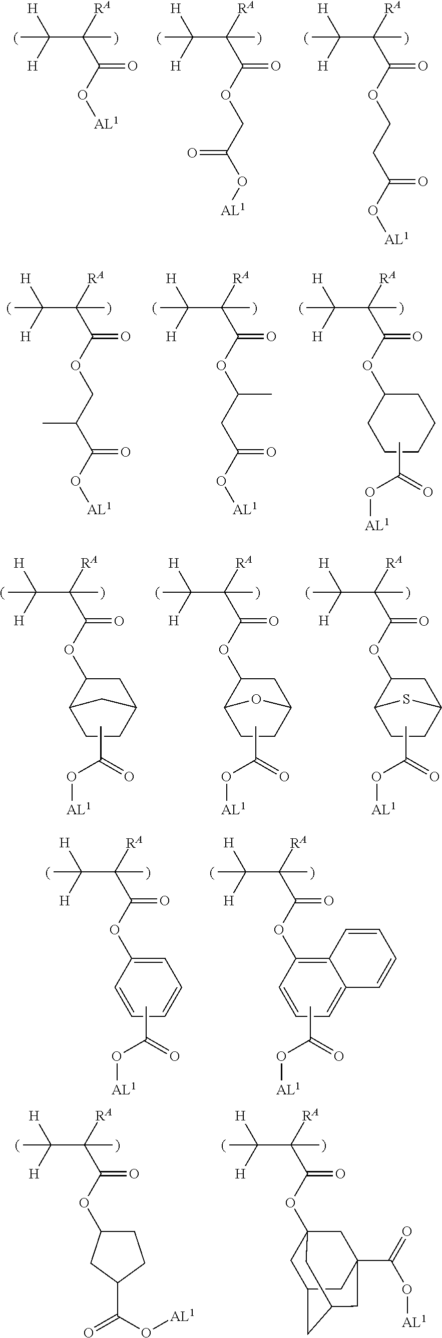

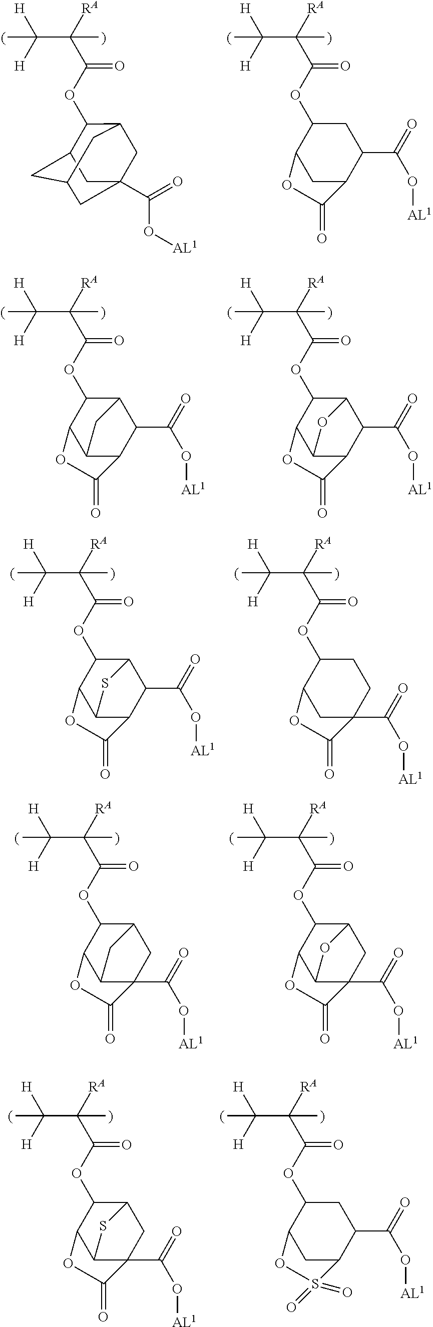

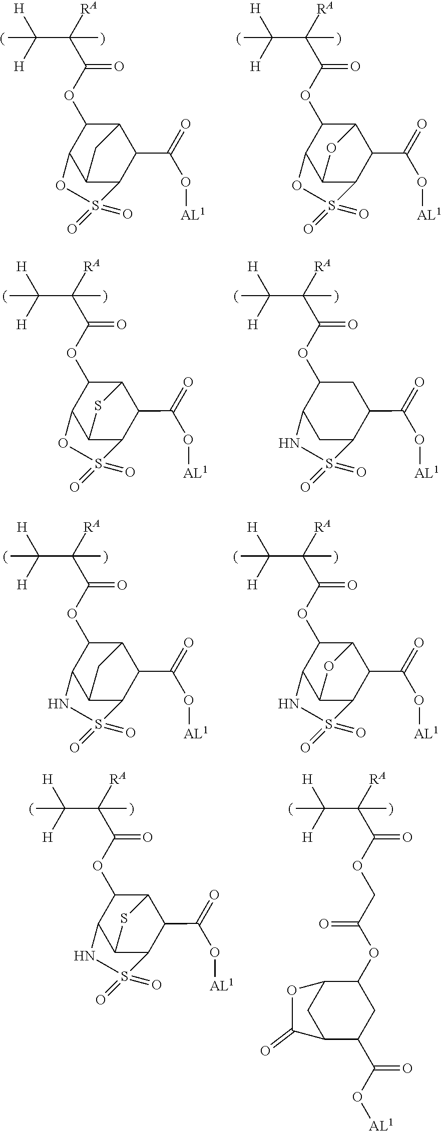

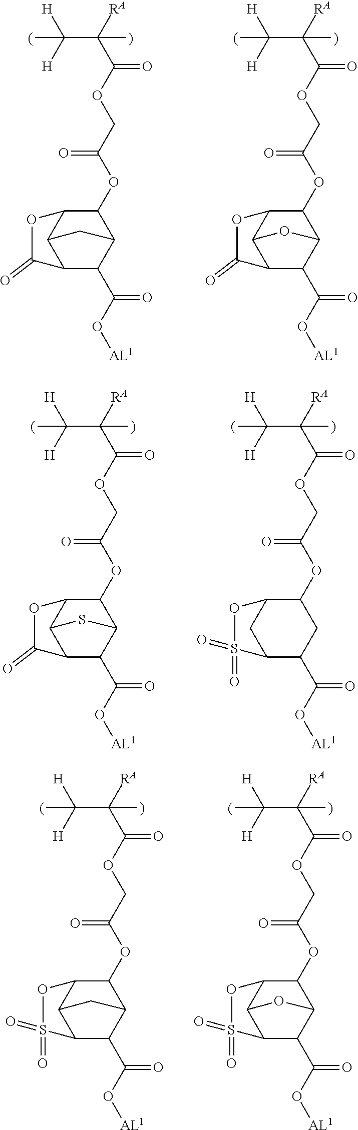

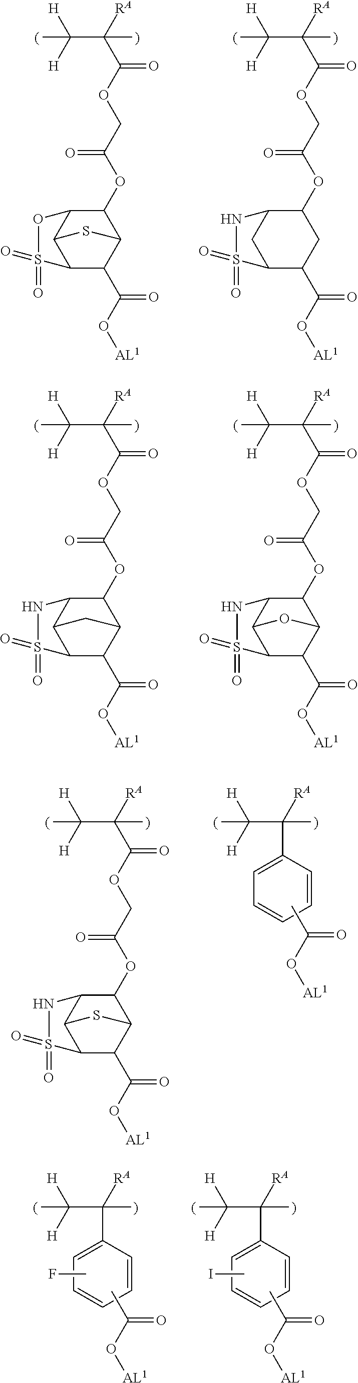

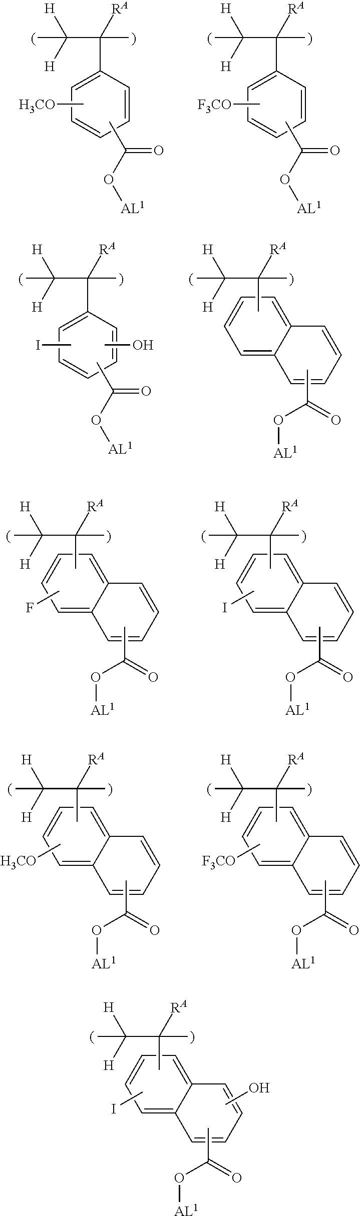

Specific examples of the repeating unit-a2 include, but are not limited to, the following. Note that in the following formulae, R^{A} and AL² are as defined above.

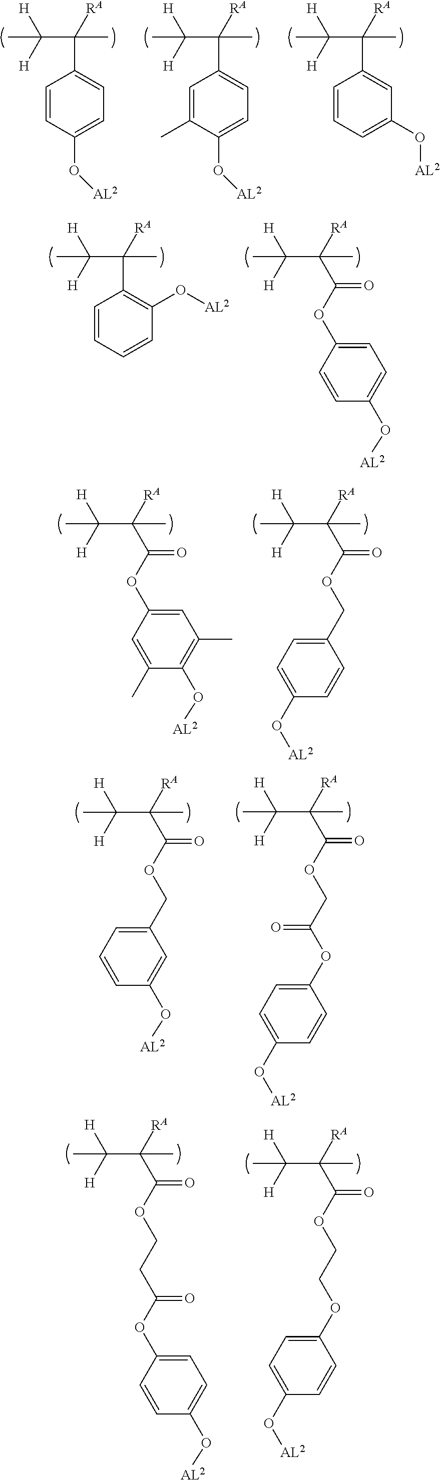

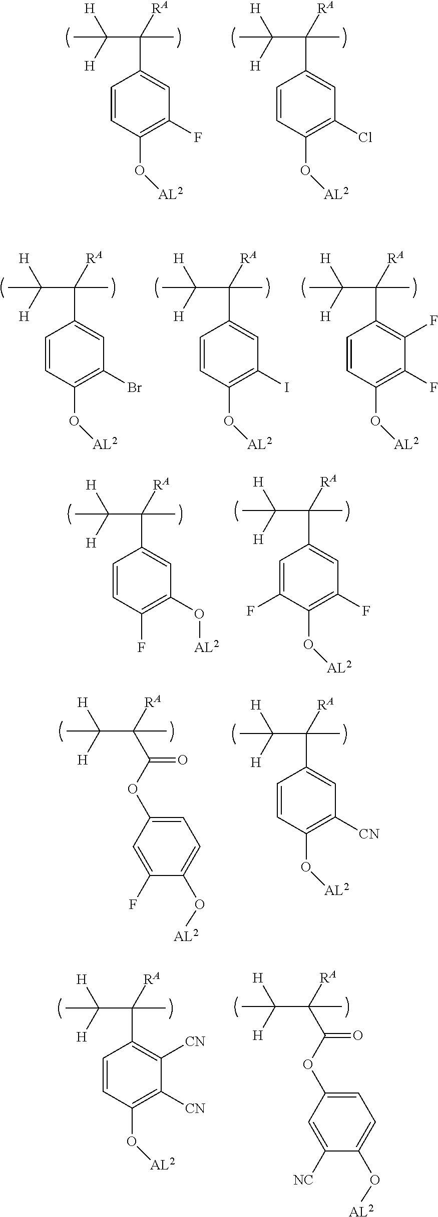

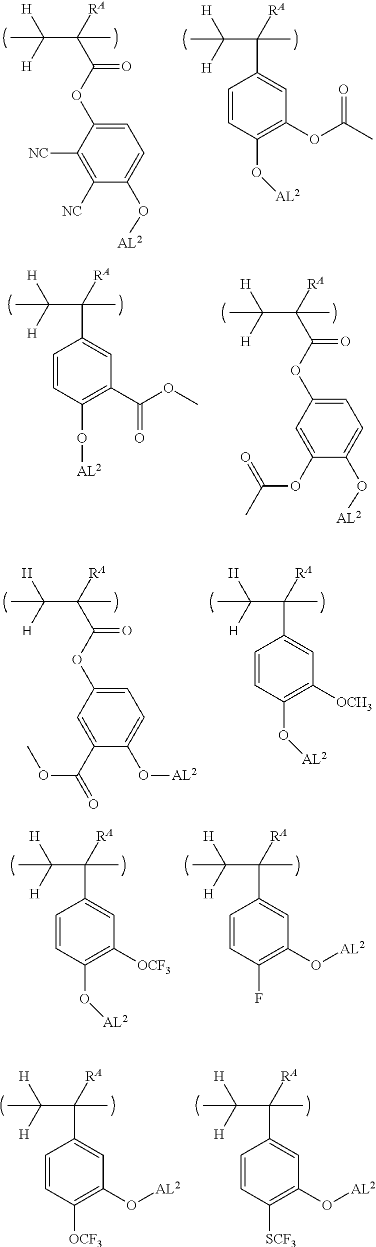

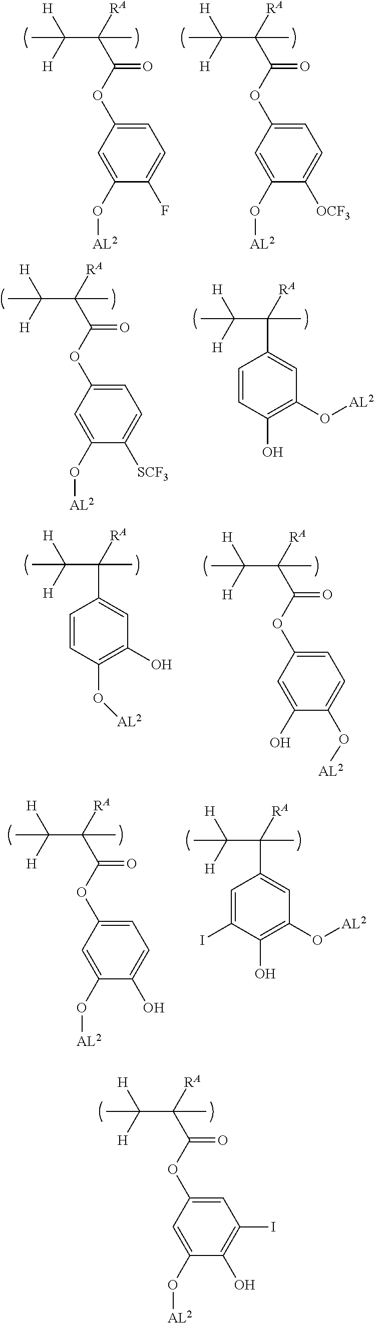

The base polymer may further contain a repeating unit represented by the following general formula (a3) (hereinafter also referred to as “repeating unit-a3”). That is, it is preferable that the base polymer further contains a repeating unit represented by the following general formula (a3).

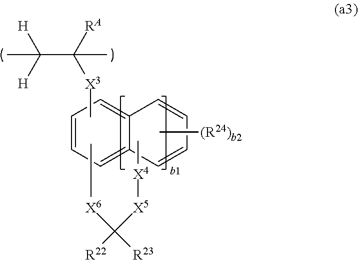

In the formula, “b1” represents 0 or 1; “b2” represents an integer from 0 to 3 when “b1” is 0, and an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; * represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; however, X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; furthermore, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, a plurality of R²⁴ may be bonded to each other to form a ring together with the carbon atoms of the aromatic ring to which they are bonded.

In the general formula (a3), “b1” represents 0 or 1. When “b1” is 0, the structure is a benzene ring, and when “b1” is 1, it is a naphthalene ring; however, from the viewpoint of solvent solubility, it is preferable that “b1” is 0, giving a benzene ring. “b2” represents an integer from 0 to 3 when “b1” is 0, and represents an integer from 0 to 5 when “b1” is 1. From the viewpoint of raw material procurement, “b2” is preferably 0, 1, 2, or 3, and more preferably 0, 1, or 2.

In the general formula (a3), R^{A} represents a hydrogen atom, a fluorine atom, a methyl group, or a trifluoromethyl group. Among these, a hydrogen atom or a methyl group is preferable, and a hydrogen atom is more preferable.

In the general formula (a3), X³ represents a single bond, *—C(═O)—O—, or *—C(═O)—NH—. * represents an attachment point to a main chain carbon atom. Among these, a single bond or *—C(═O)—O— is preferable, and a single bond is more preferable.

In the general formula (a3), X⁴ represents a single bond, an aliphatic hydrocarbylene group having 1 to 4 carbon atoms, a carbonyl group, a sulfonyl group, or a group obtained by combining these. Among these, from the viewpoint of raw material procurement, a single bond, a carbonyl group, or a sulfonyl group is preferable, and from the viewpoint of a polar group generated after the reaction, a single bond or a carbonyl group is more preferable.

In the general formula (a3), X⁵ and X⁶ each independently represent an oxygen atom or a sulfur atom. However, X⁴ and X⁶ are bonded to adjacent carbon atoms of an aromatic ring. X⁵ and X⁶ may be the same as or different from each other, but from the viewpoint of reactivity, X⁵ and X⁶ are both preferably oxygen atoms.

In the general formula (a3), R²² and R²³ each independently represent a hydrogen atom or a hydrocarbyl group having 1 to 20 carbon atoms and optionally containing a heteroatom. The hydrocarbyl group may be saturated or unsaturated and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, allyl, propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl and naphthyl groups; aralkyl groups having 7 to 20 carbon atoms, such as benzyl, 1-phenylethyl, and 2-phenylethyl groups; and groups obtained by combining these, etc. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Furthermore, R²² and R²³ may be bonded to each other to form a ring together with the carbon atom to which they are bonded. Specific examples of the ring formed in this case include a cyclopropane ring, a cyclobutane ring, a cyclopentane ring, a cyclohexane ring, a norbornane ring, an adamantane ring, etc. In addition, some or all of the hydrogen atoms of the above-described rings may be substituted with a group containing a heteroatom such as an oxygen atom, a sulfur atom, a nitrogen atom, or a halogen atom, and some of the —CH₂— of the above-described rings may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In the general formula (a3), R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}) R^{24A} and R^{24B} each independently represent a hydrogen atom or a hydrocarbyl group having 1 to 6 carbon atoms. The halogen atom is preferably a fluorine atom, a chlorine atom, a bromine atom, or an iodine atom, and more preferably a fluorine atom or an iodine atom. The hydrocarbyl groups, and the hydrocarbyl moieties of the hydrocarbyloxy groups, the hydrocarbyloxycarbonyl groups, and the hydrocarbylthio groups may be saturated or unsaturated and may be linear, branched, or cyclic. Specific examples thereof include the same ones as those exemplified as the hydrocarbyl group represented by R²² and R²³. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. When “b2” is 2 or more, each R²⁴ may be the same as or different from each other.

Also, when “b2” is 2 or more, a plurality of R²⁴ may be bonded to each other to form a ring together with the carbon atoms of the aromatic ring to which they are bonded. Specific examples of the ring formed in this case include a cyclopropane ring, a cyclobutane ring, a cyclopentane ring, a cyclohexane ring, a norbornane ring, an adamantane ring, etc. In addition, some or all of the hydrogen atoms of the above-described rings may be substituted with a group containing a heteroatom such as an oxygen atom, a sulfur atom, a nitrogen atom, or a halogen atom, and some of the —CH₂— of the above-described rings may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the repeating unit-a3 include, but are not limited to, the following. Note that in the following formulae, R^{A} is as defined above, and Me represents a methyl group. In addition, the bonding positions of the various substituents on the aromatic ring may be interchanged.

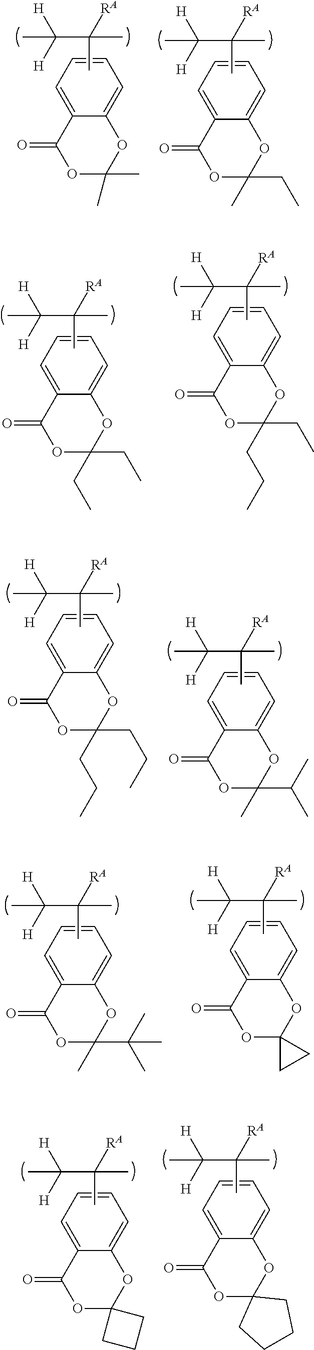

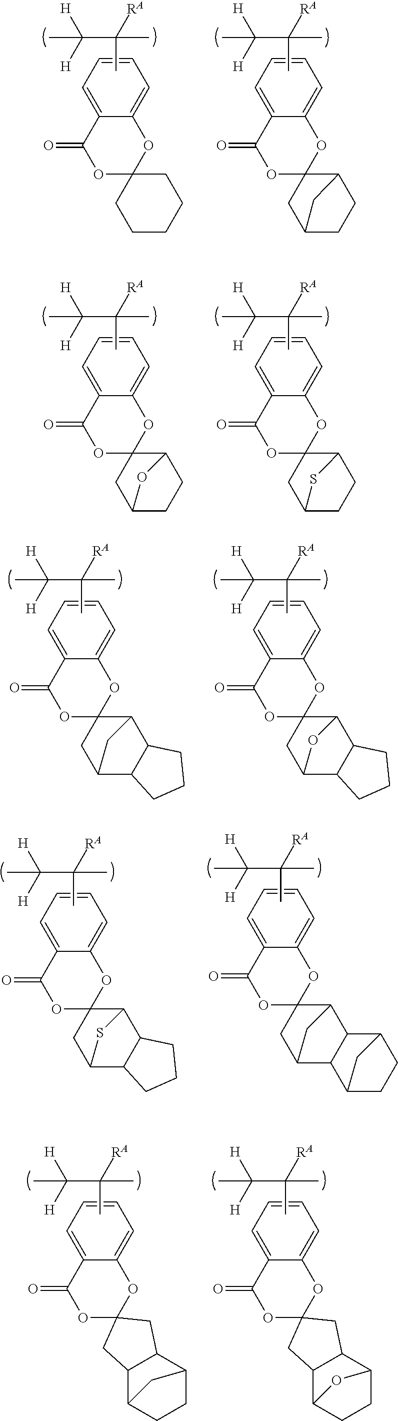

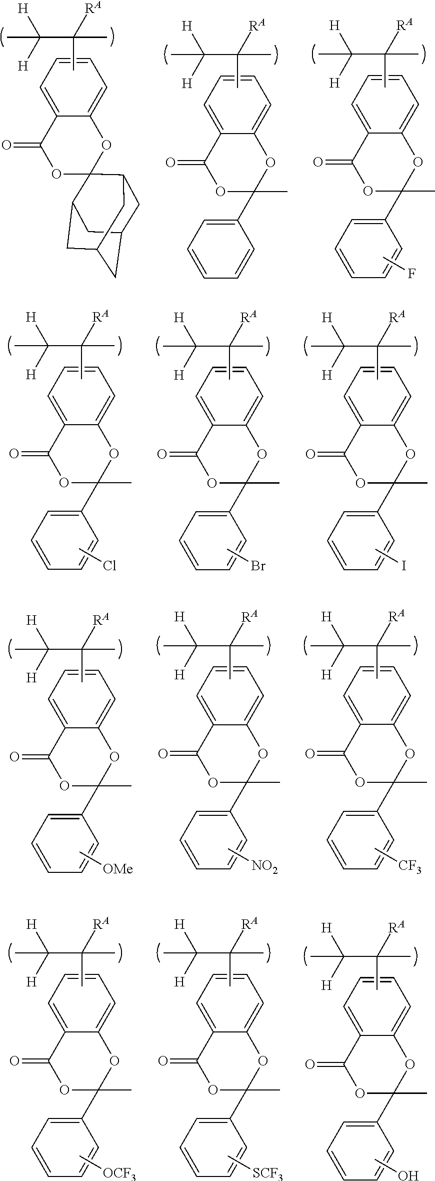

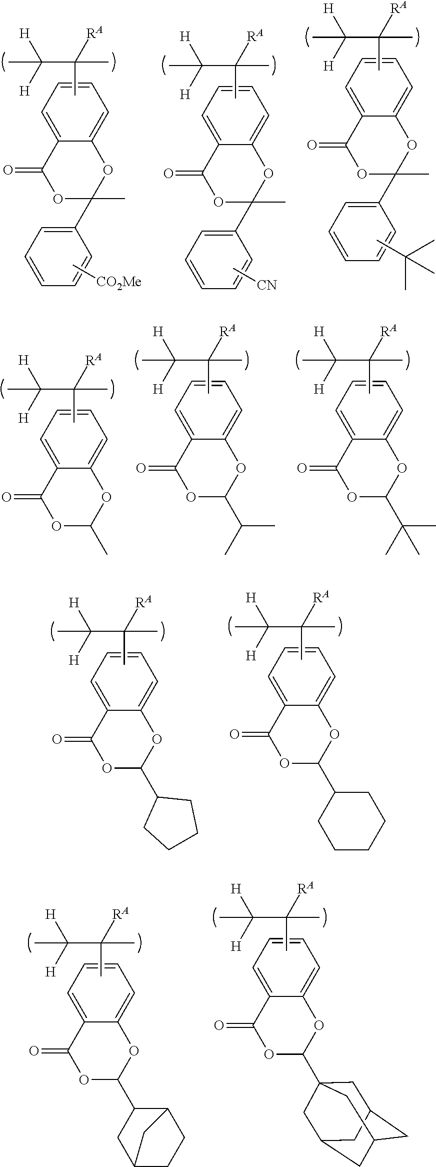

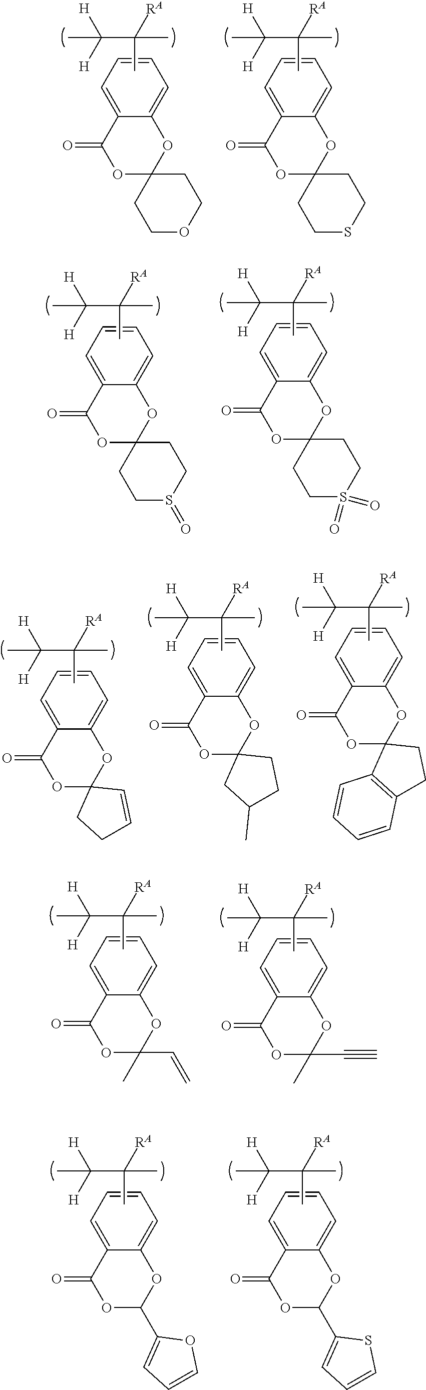

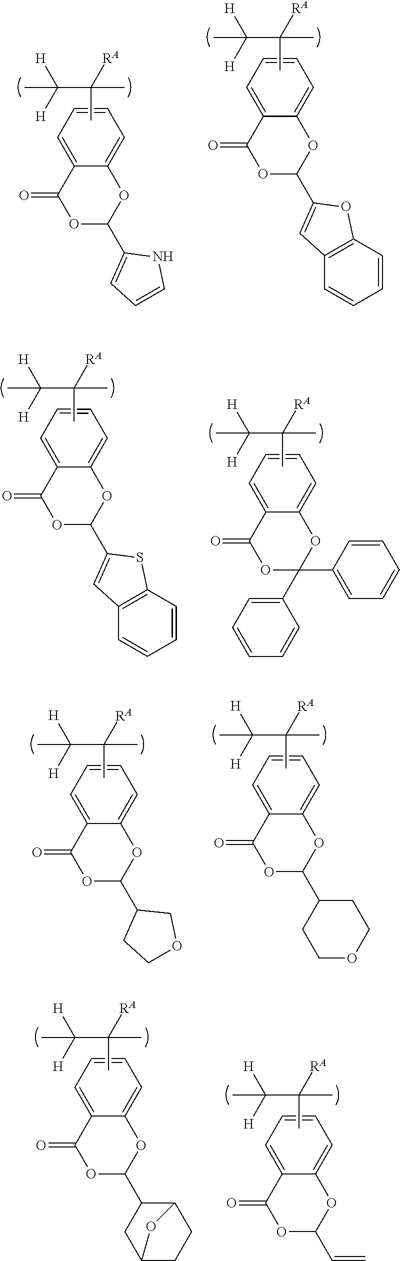

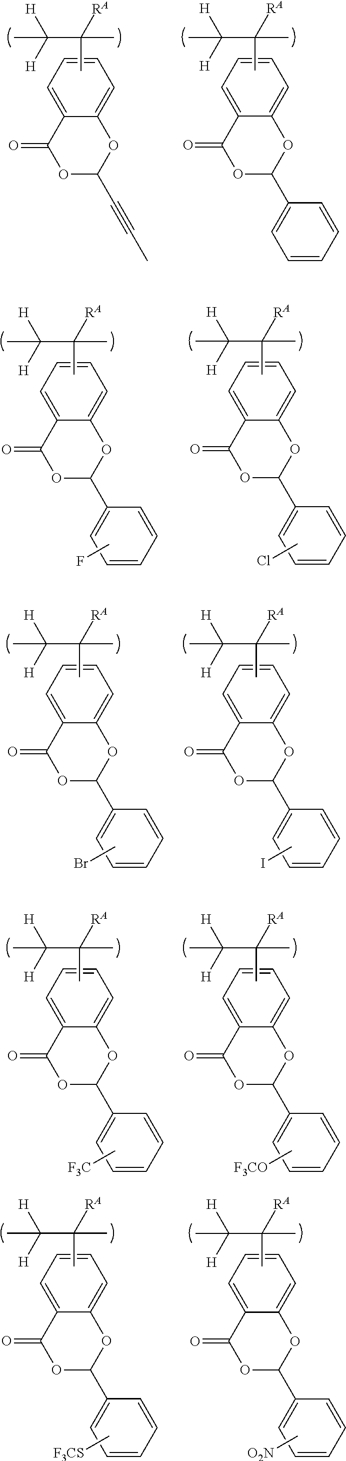

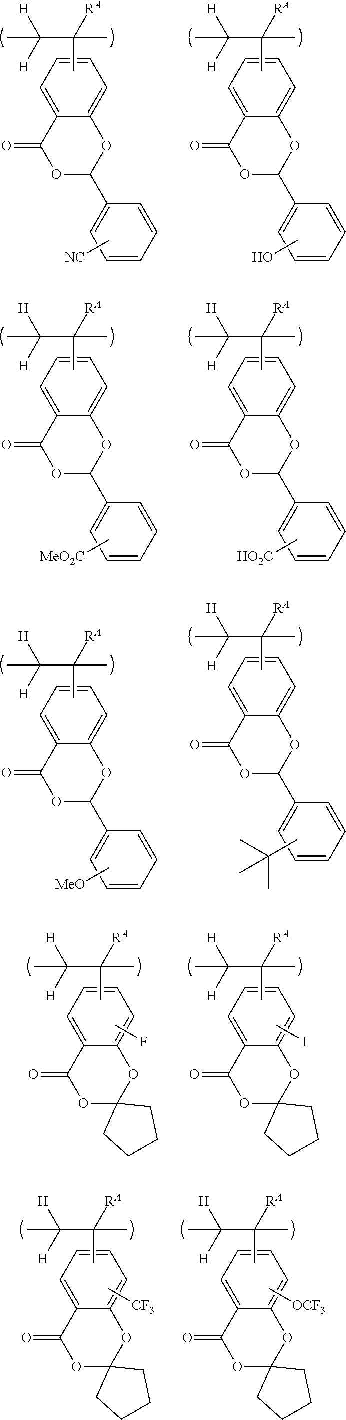

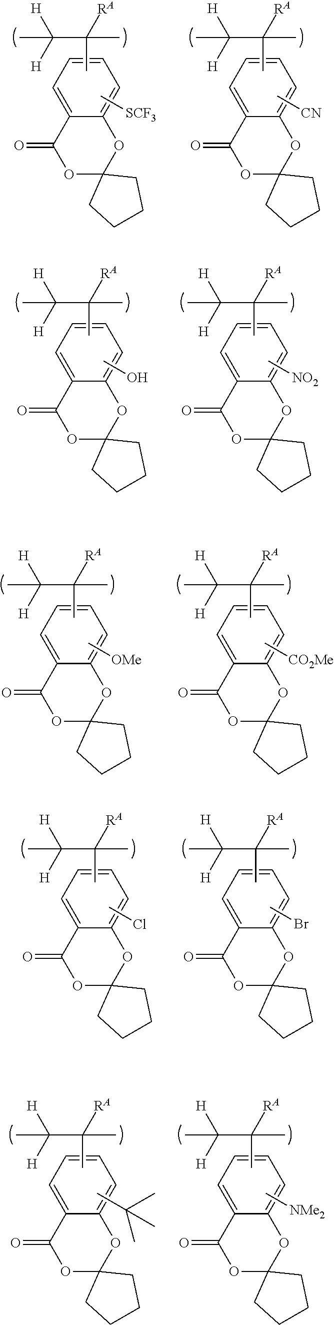

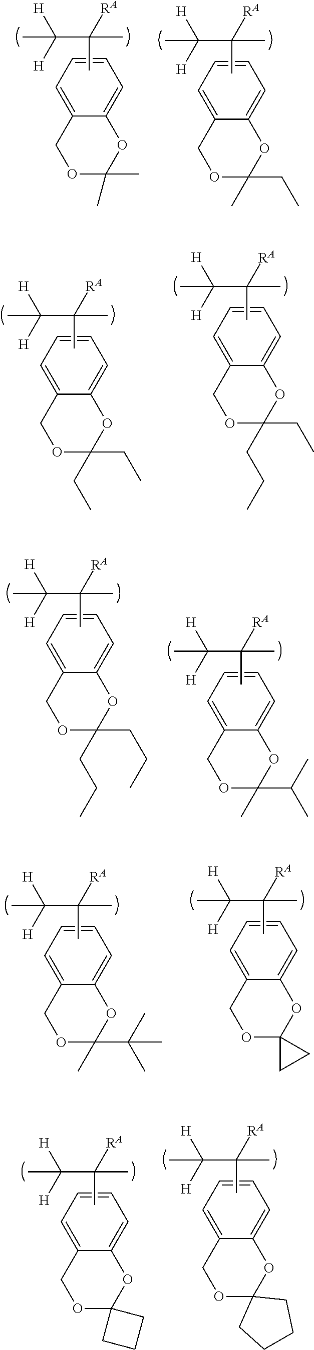

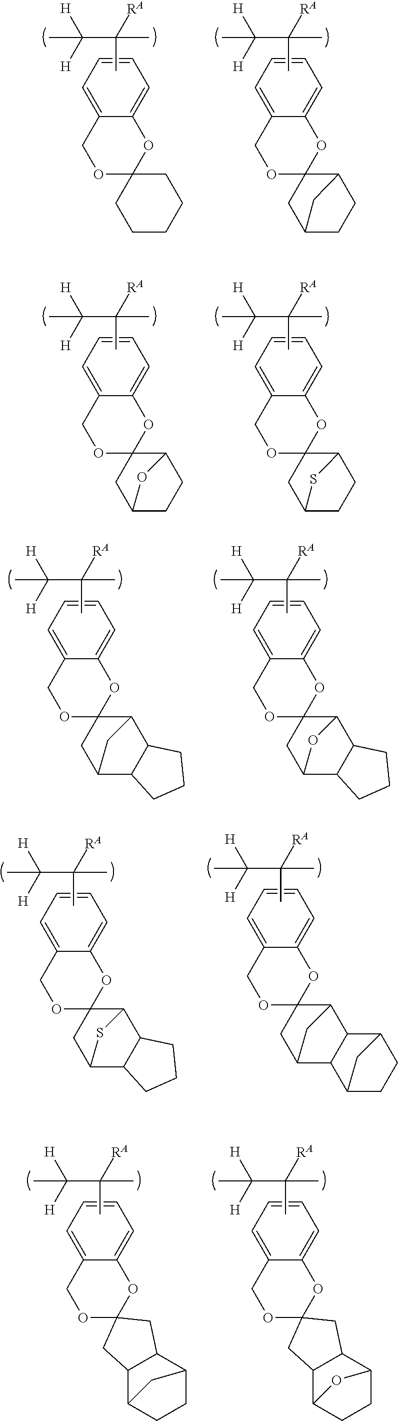

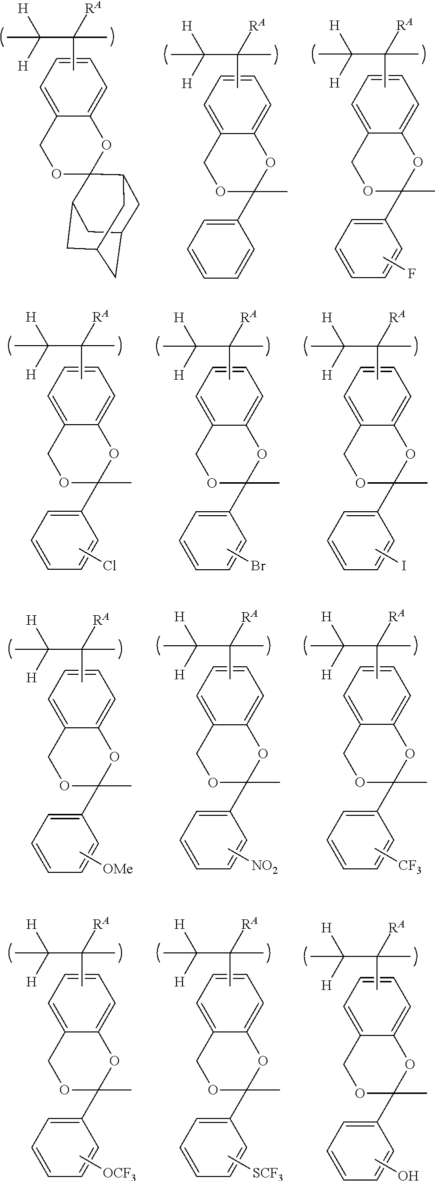

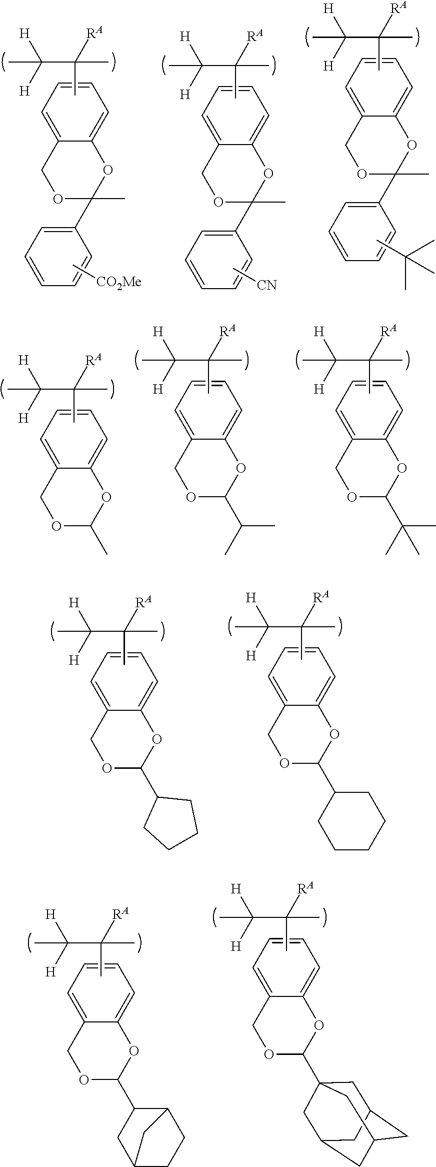

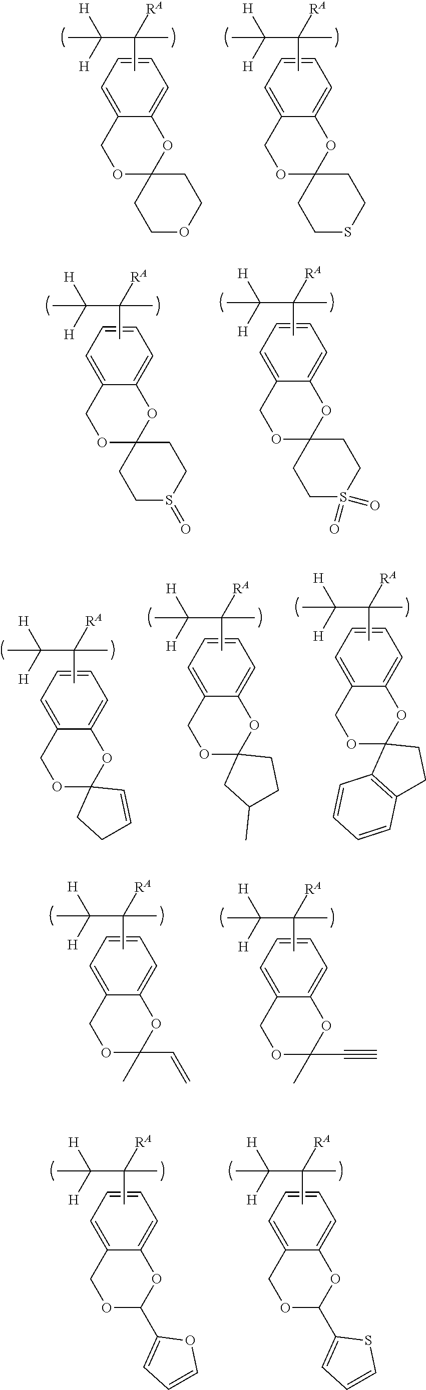

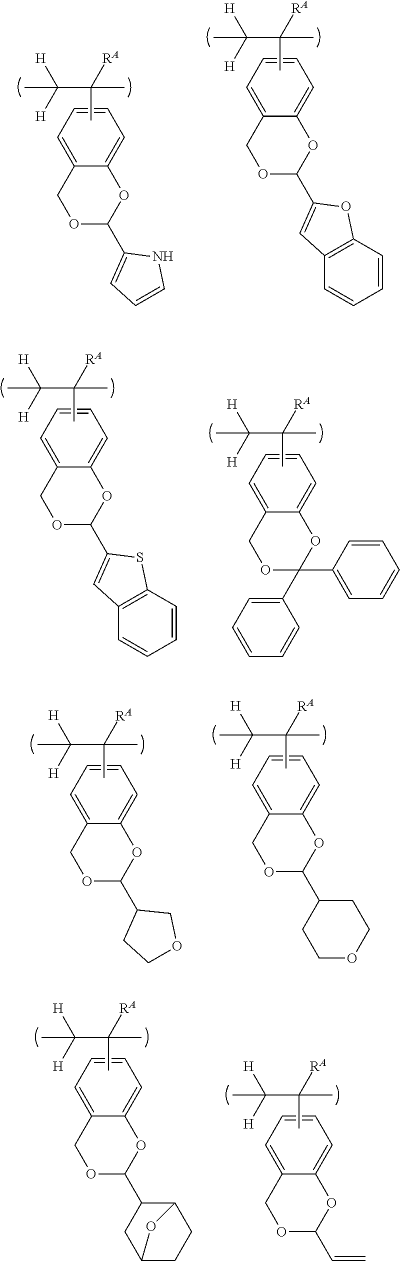

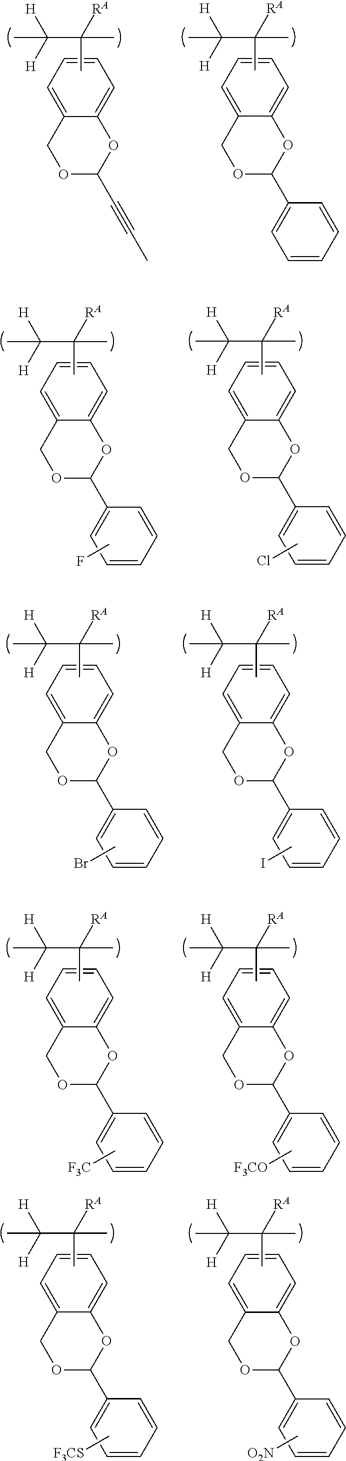

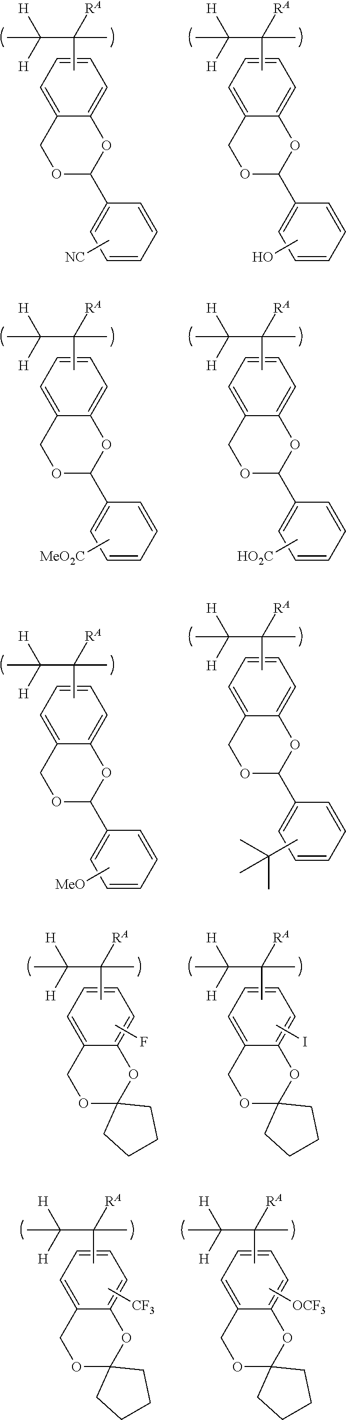

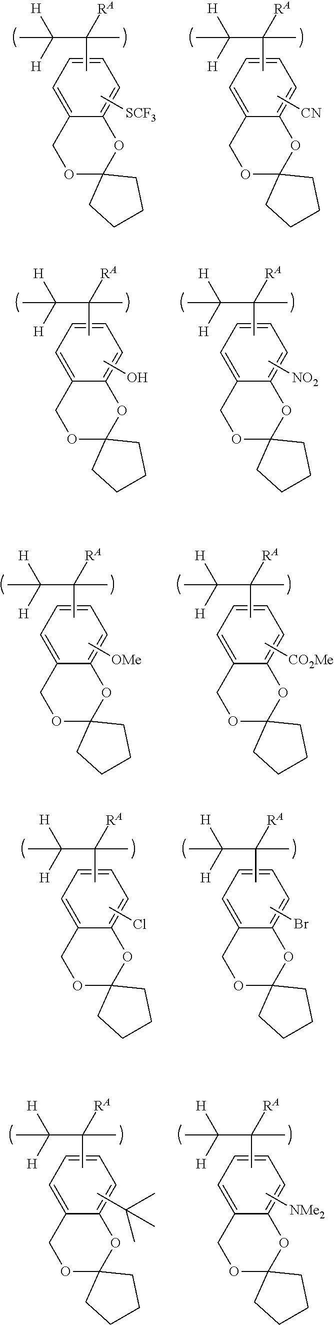

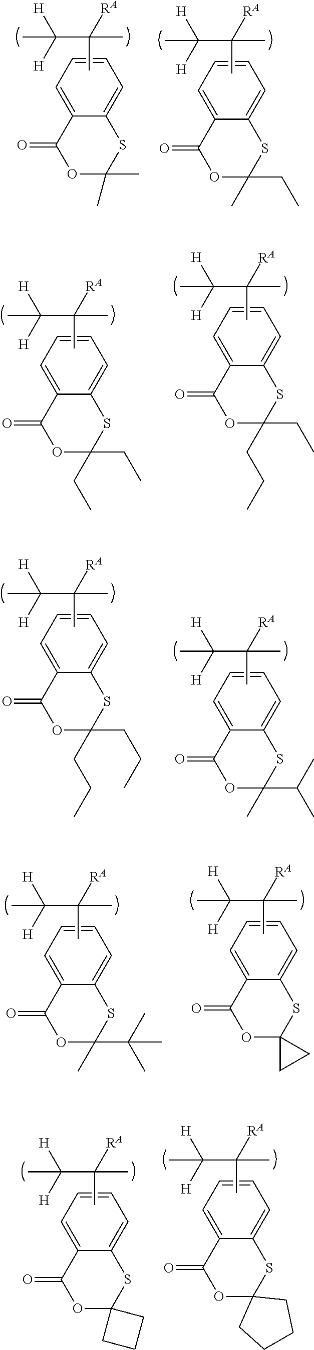

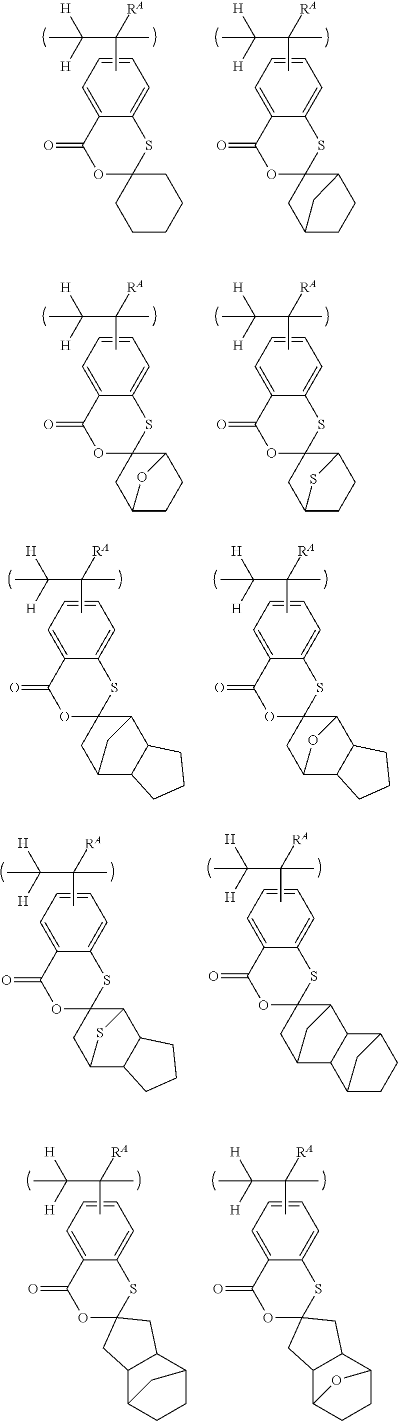

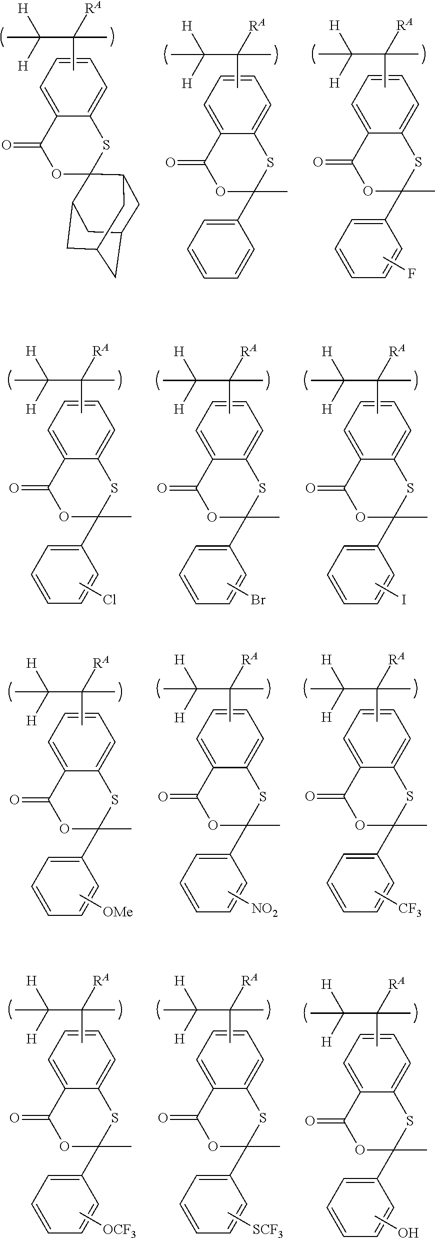

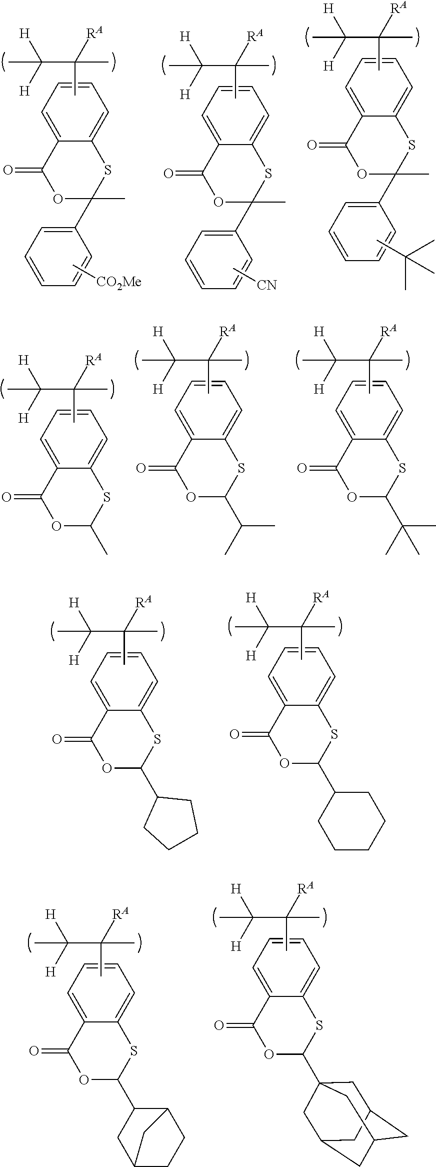

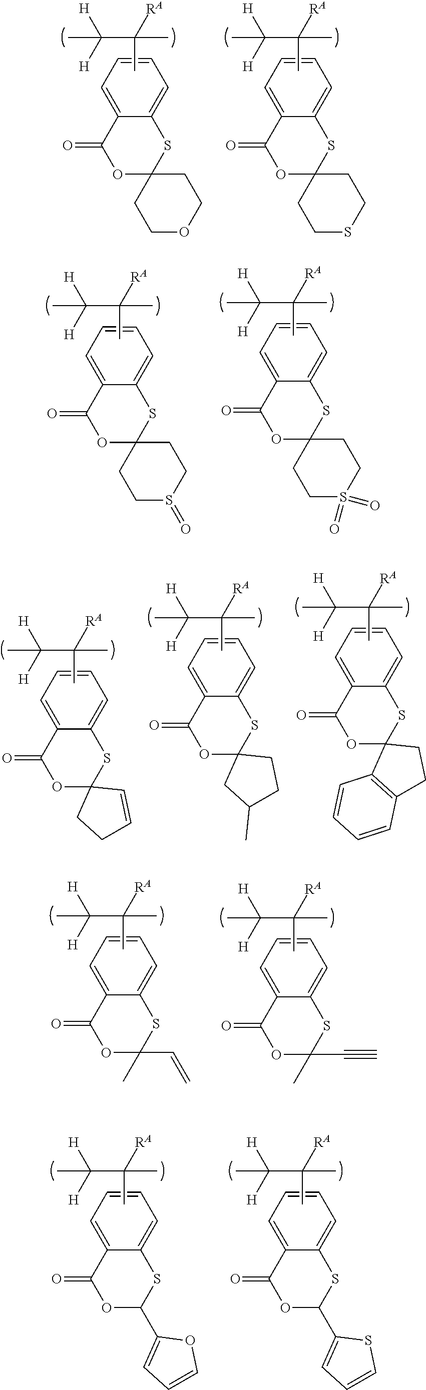

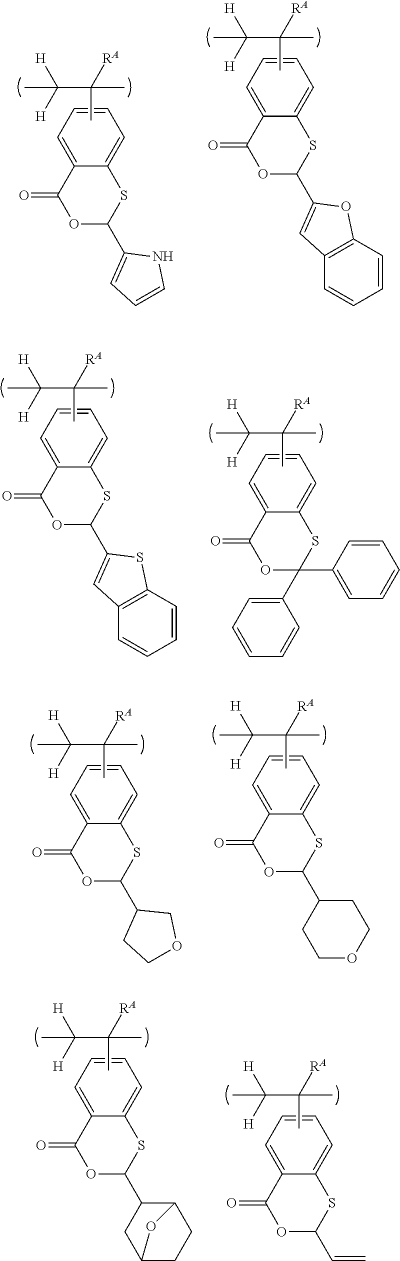

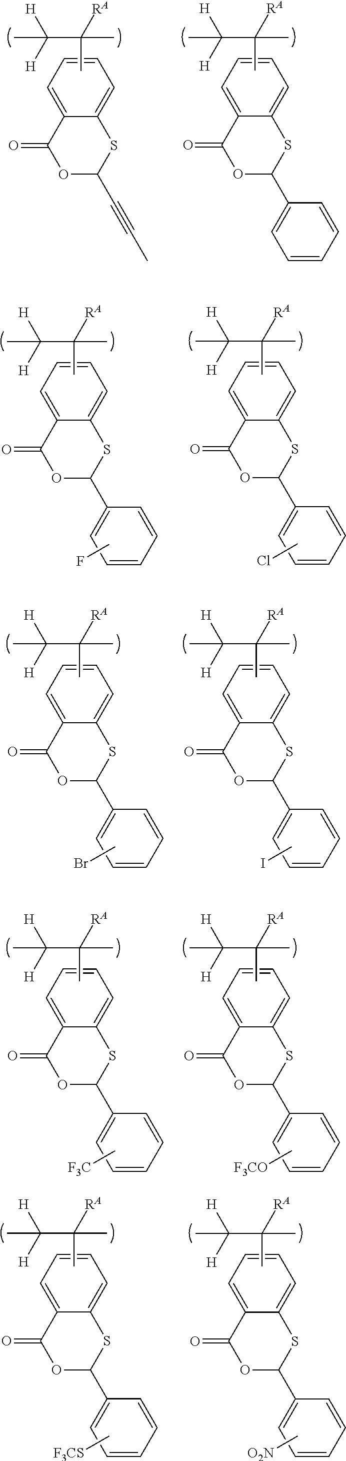

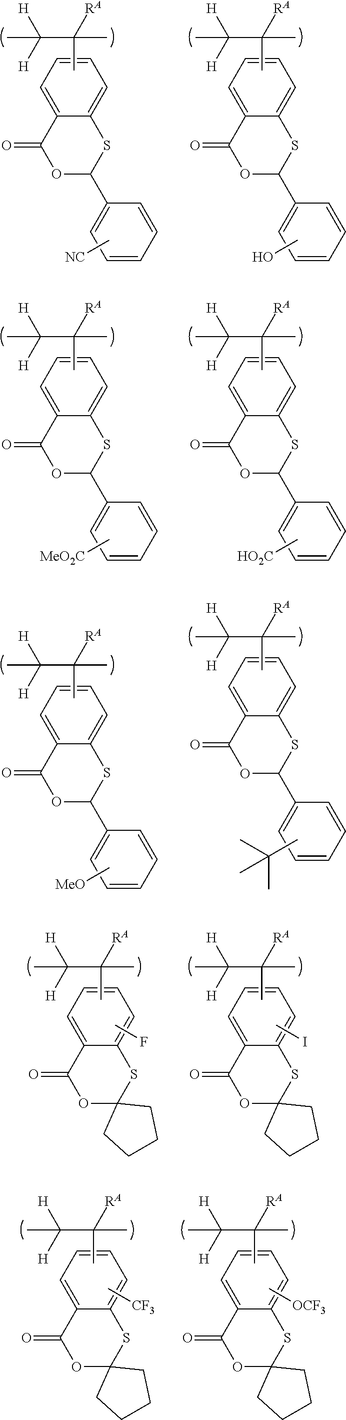

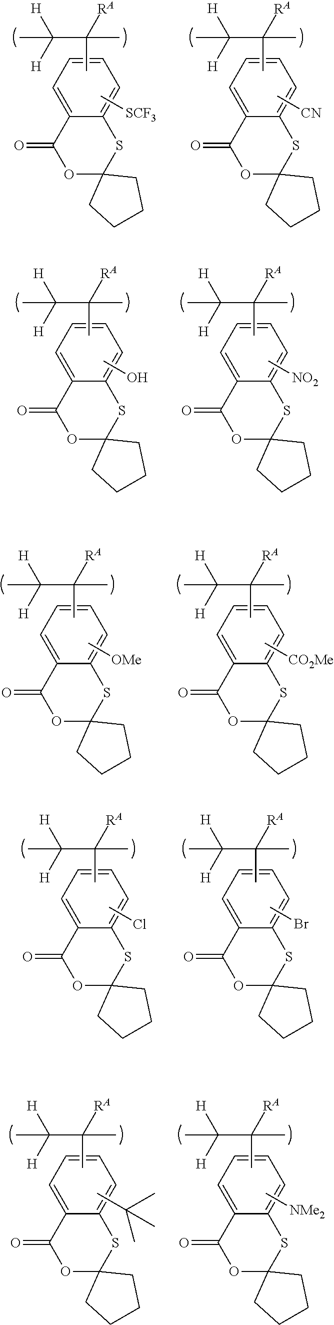

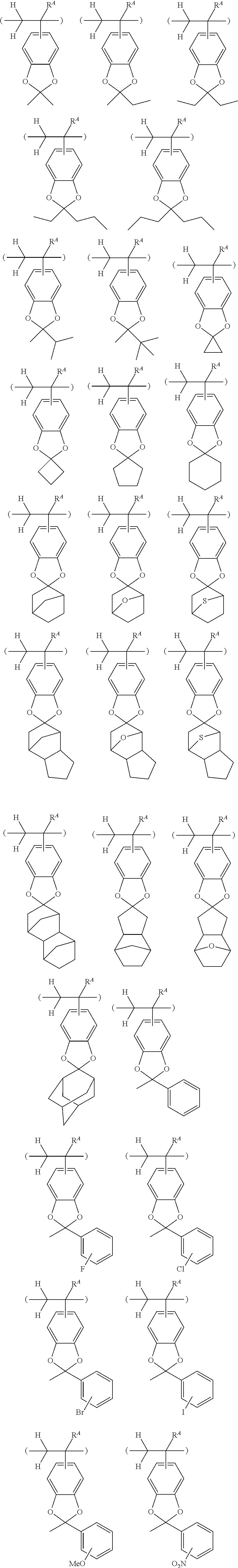

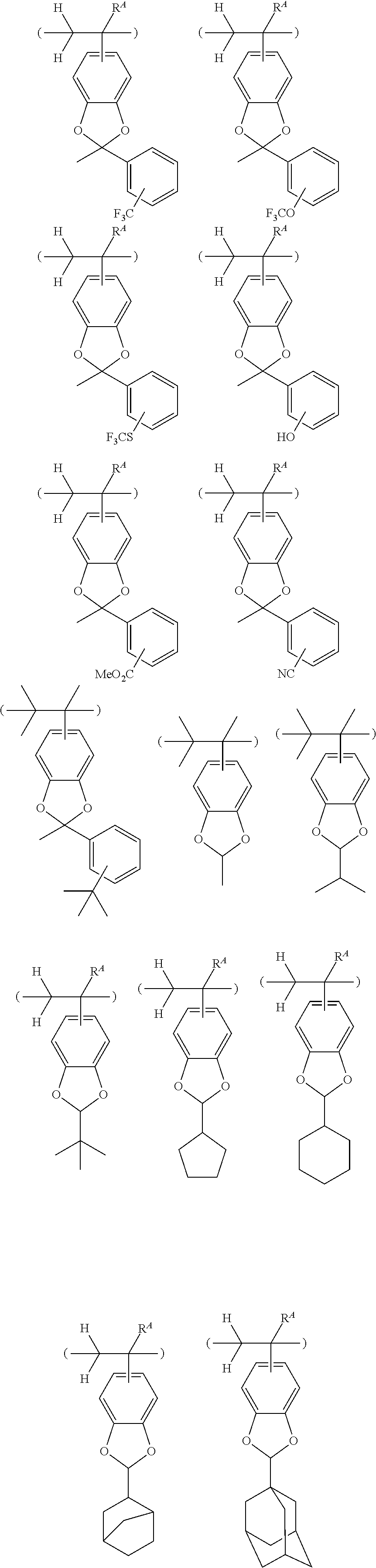

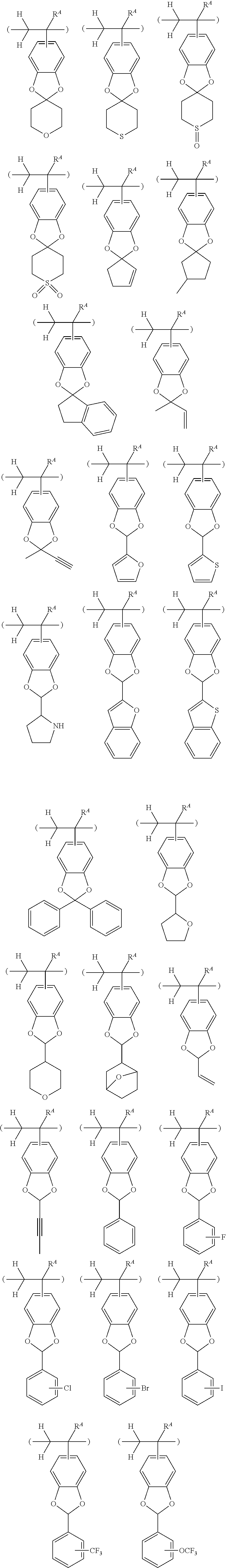

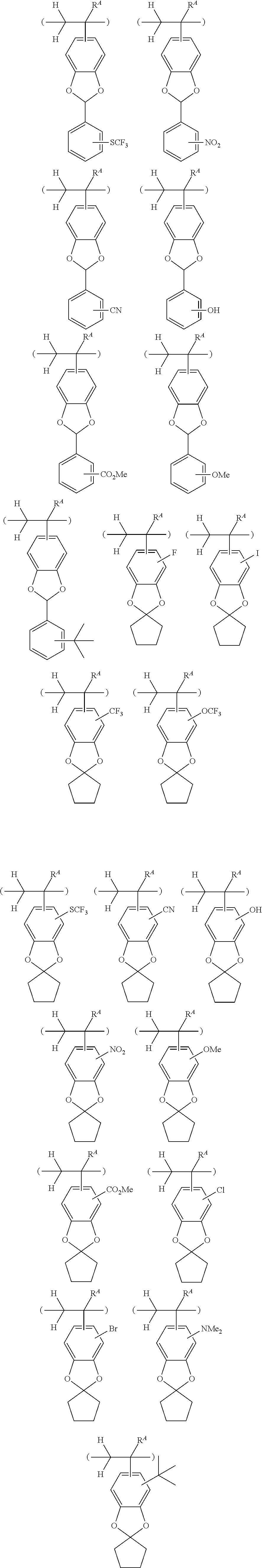

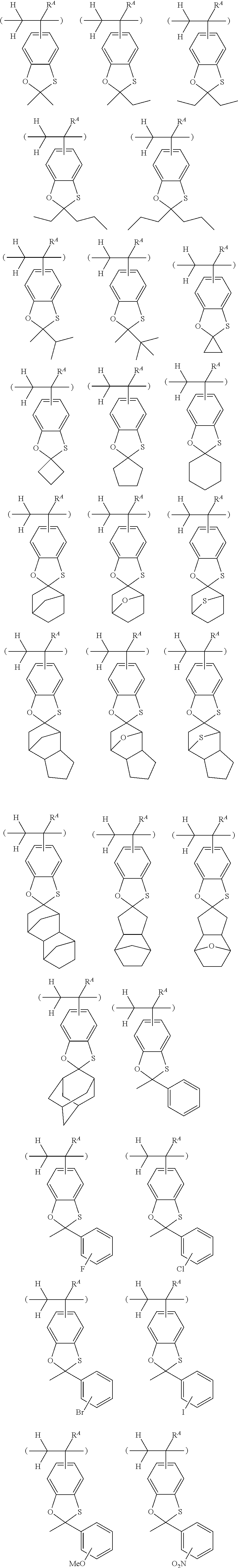

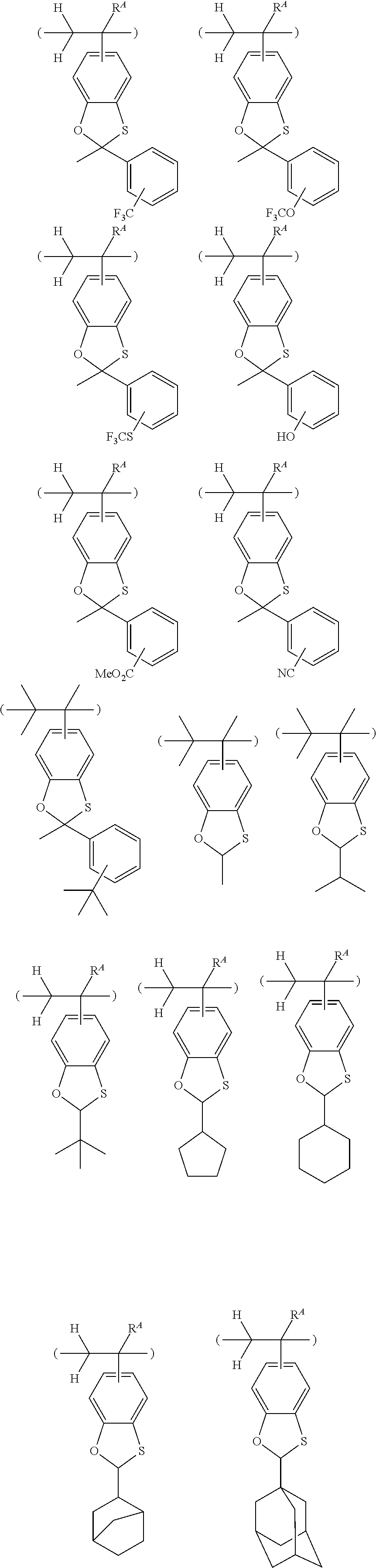

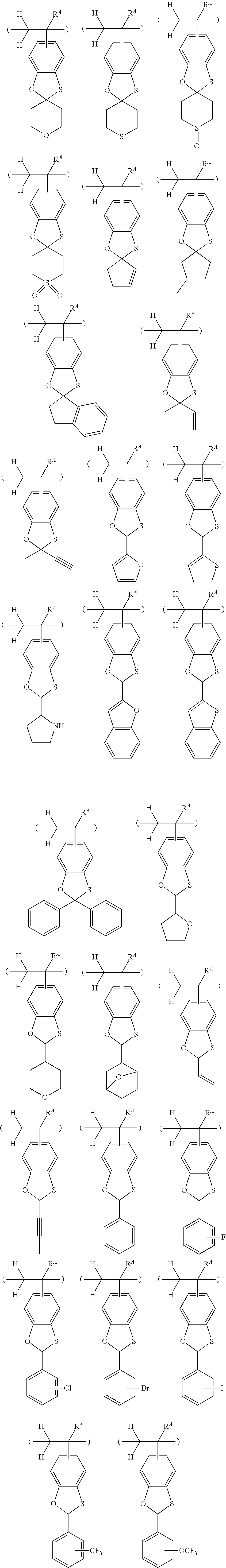

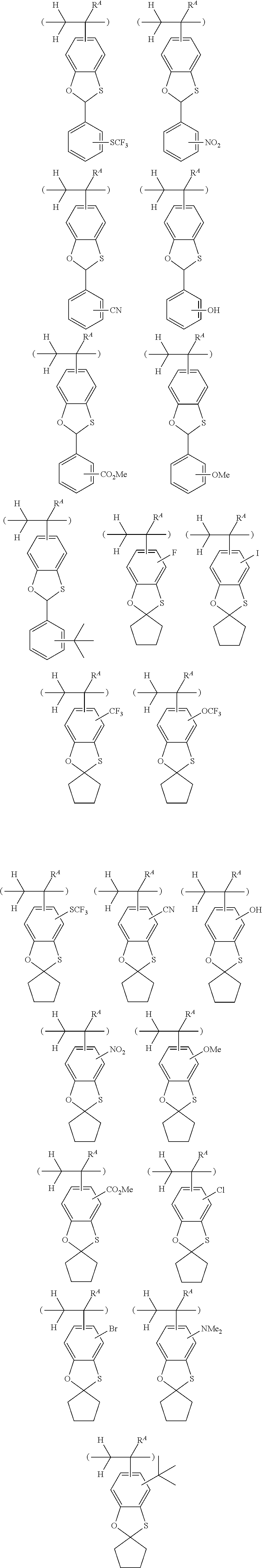

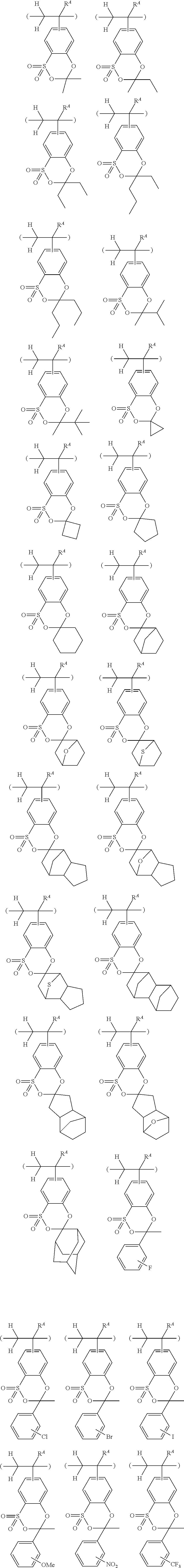

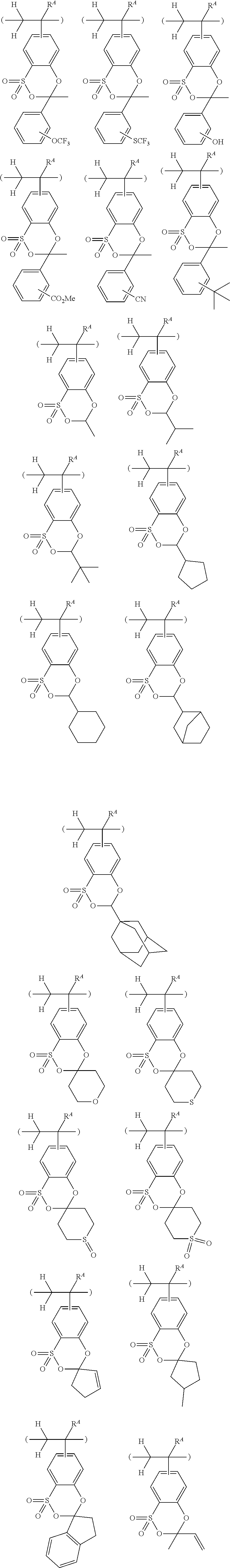

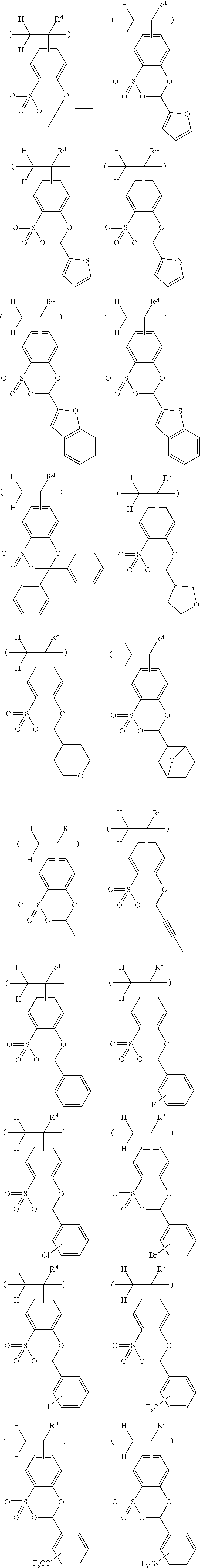

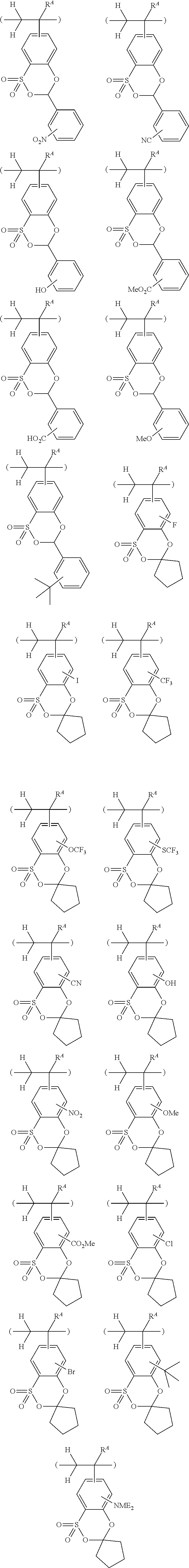

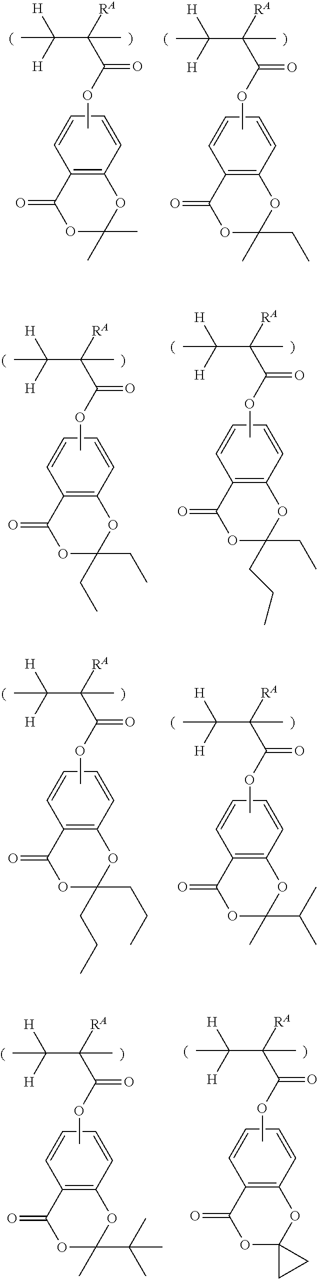

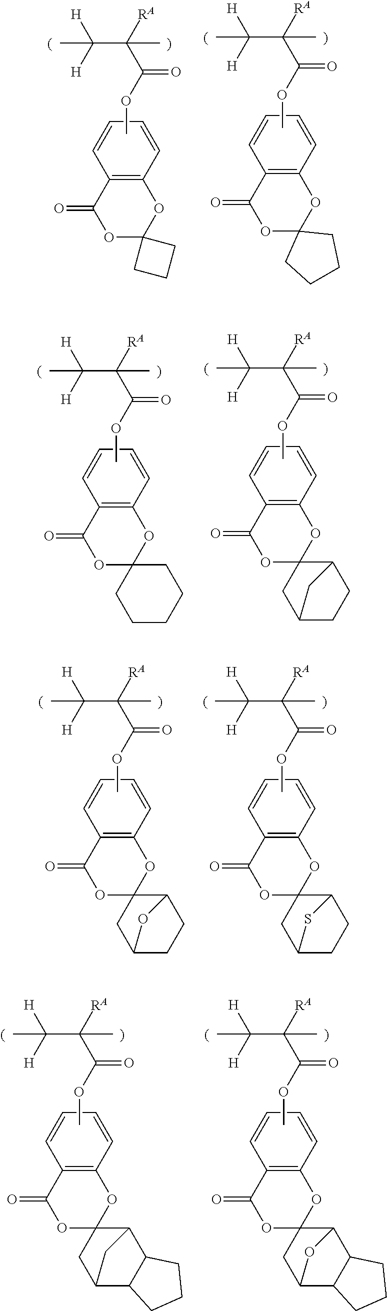

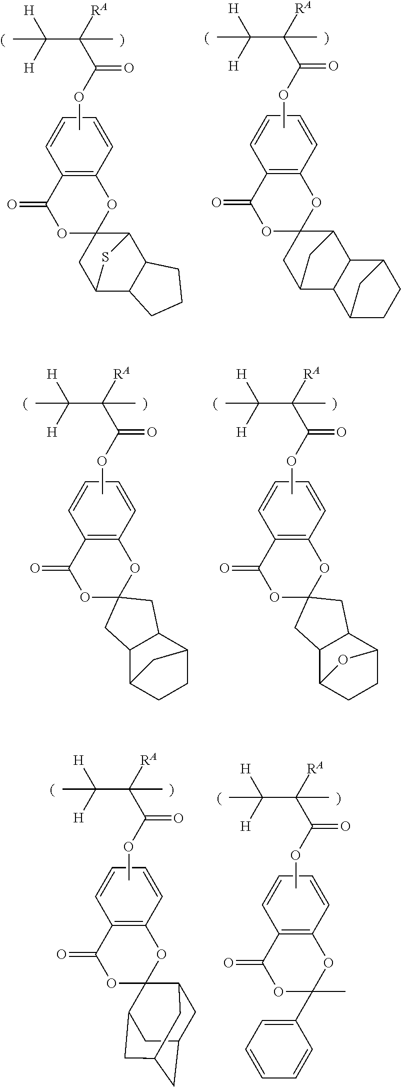

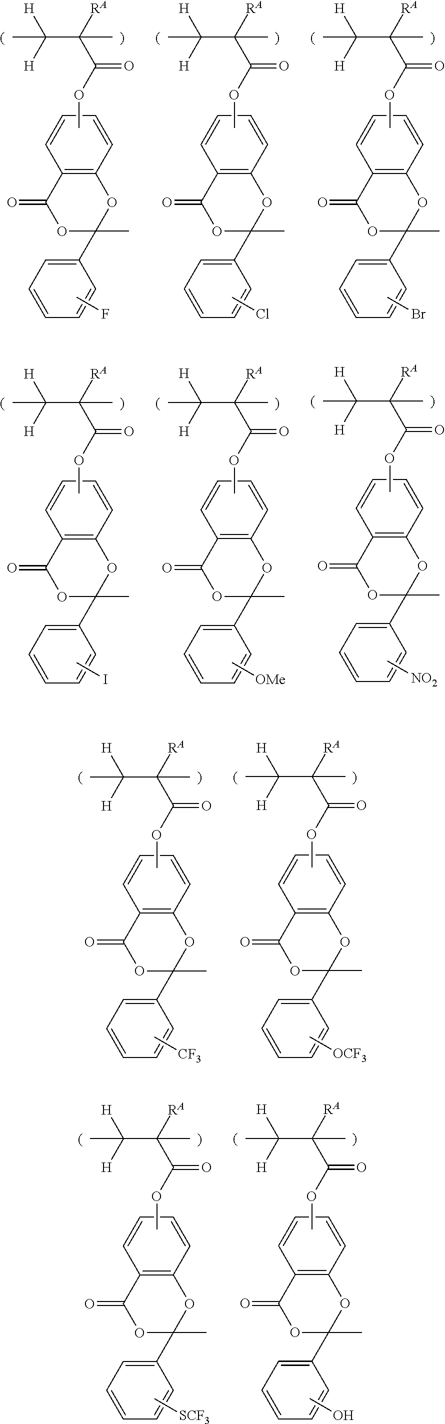

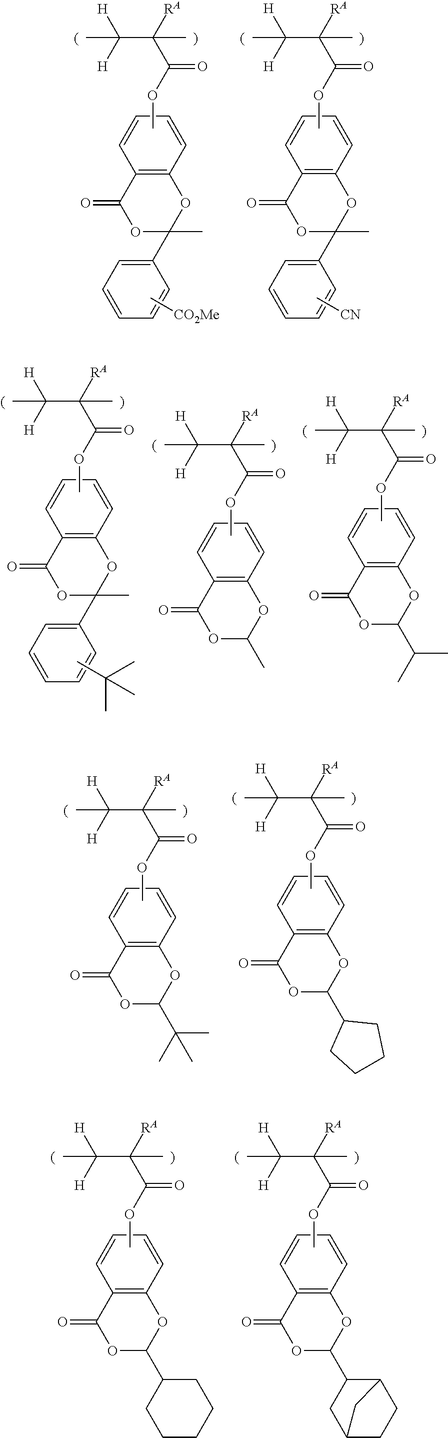

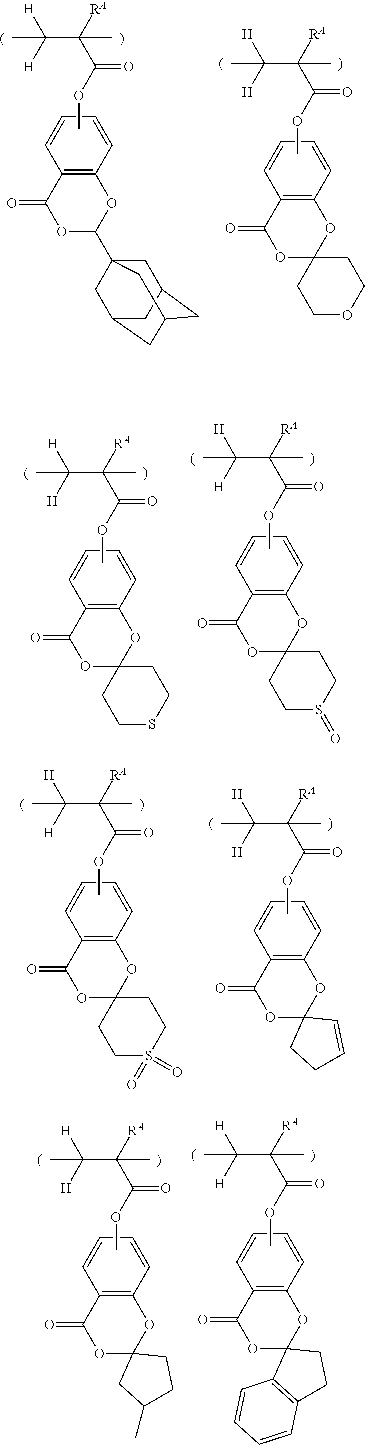

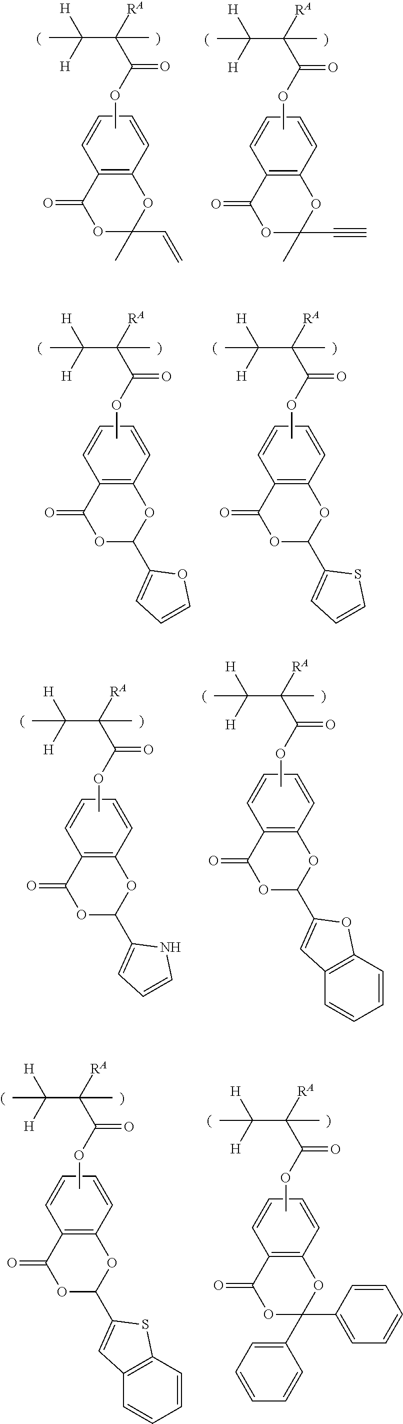

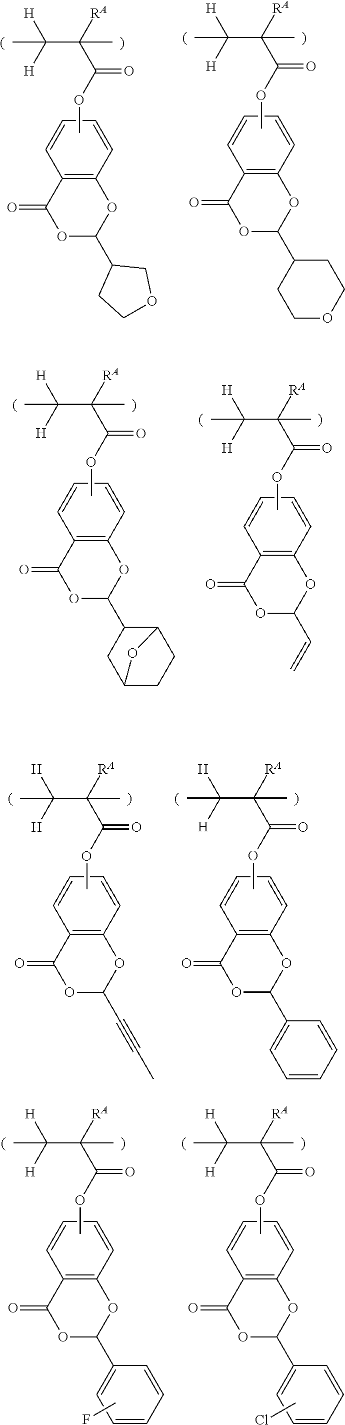

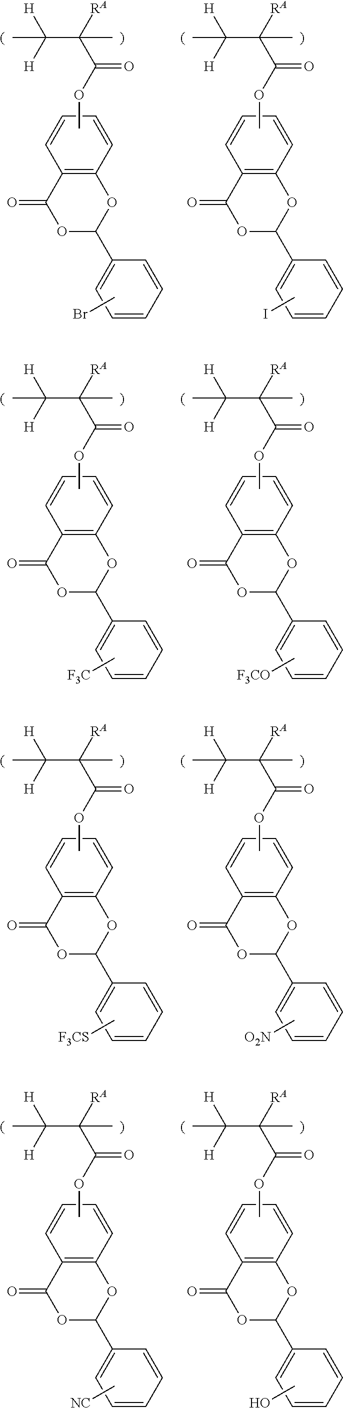

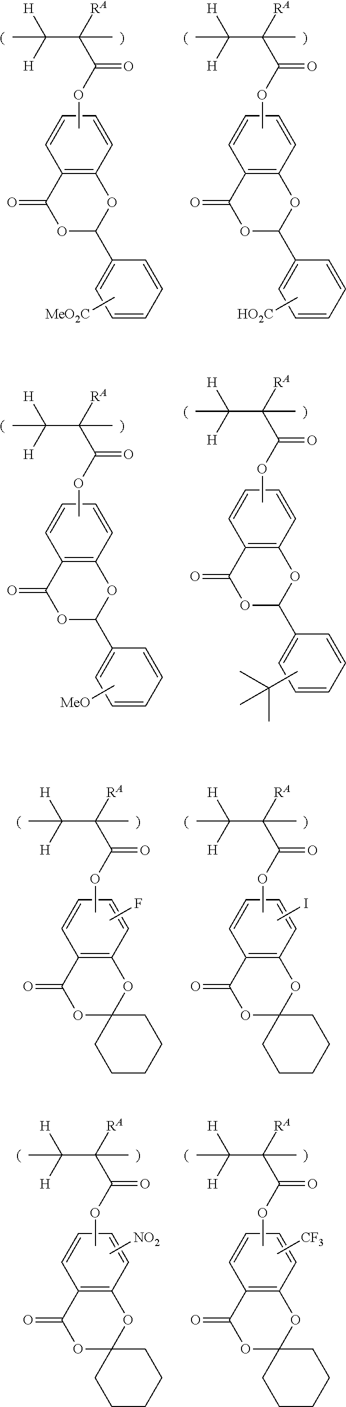

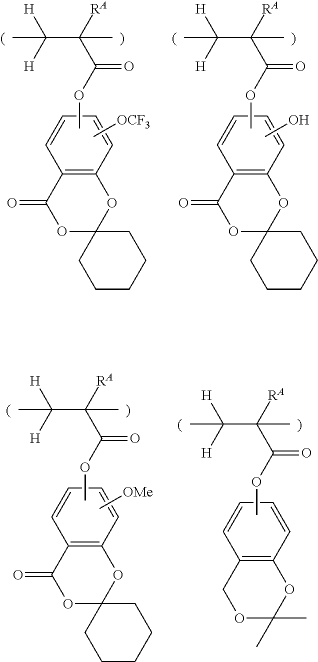

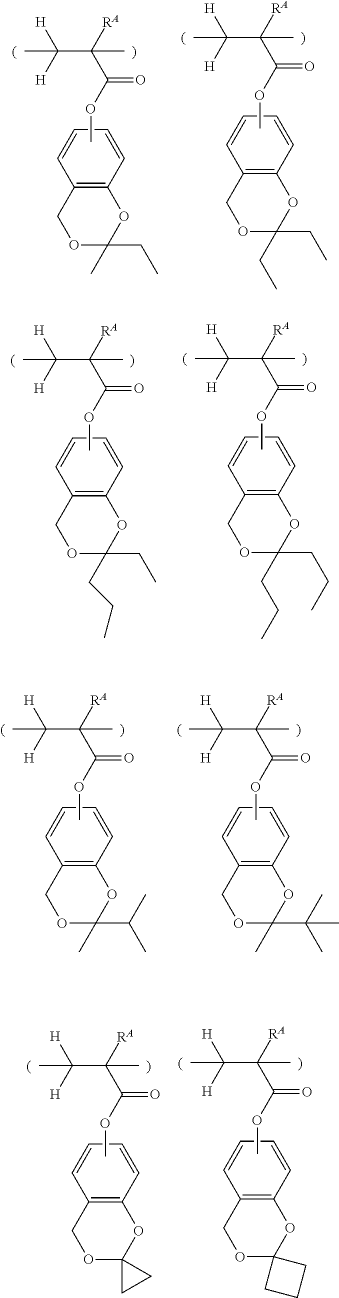

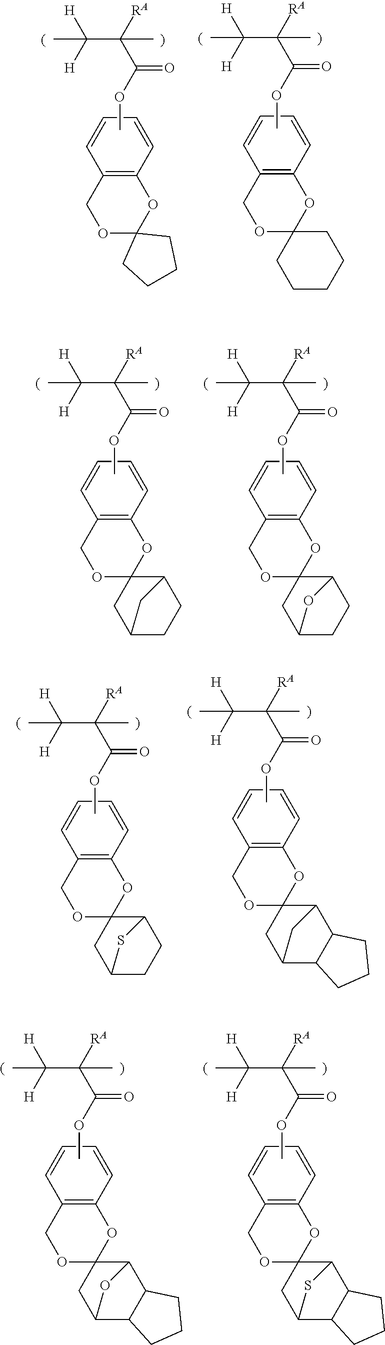

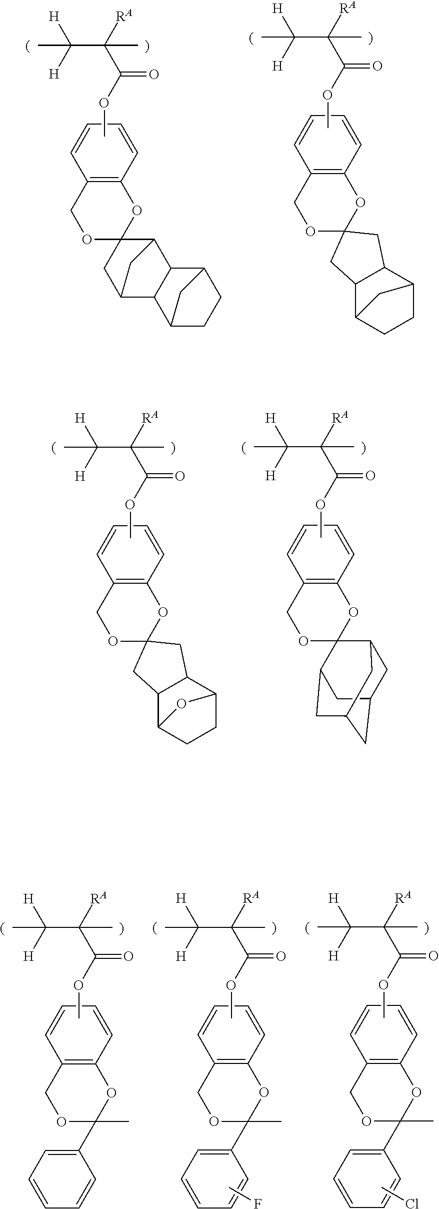

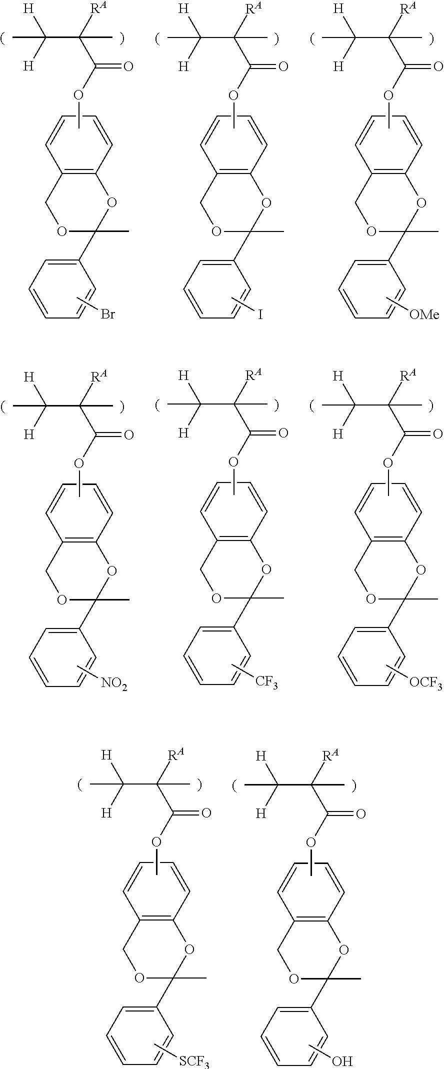

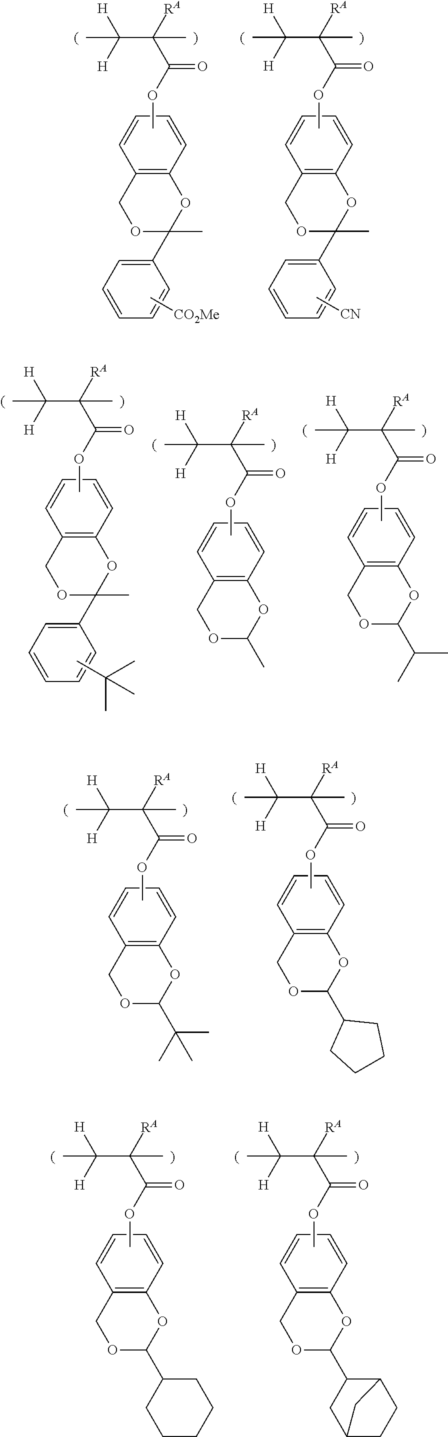

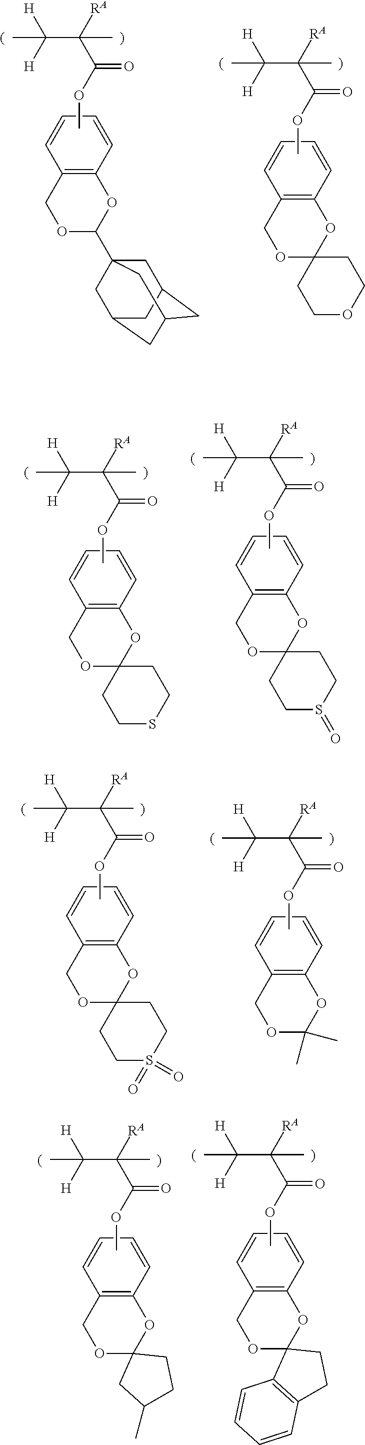

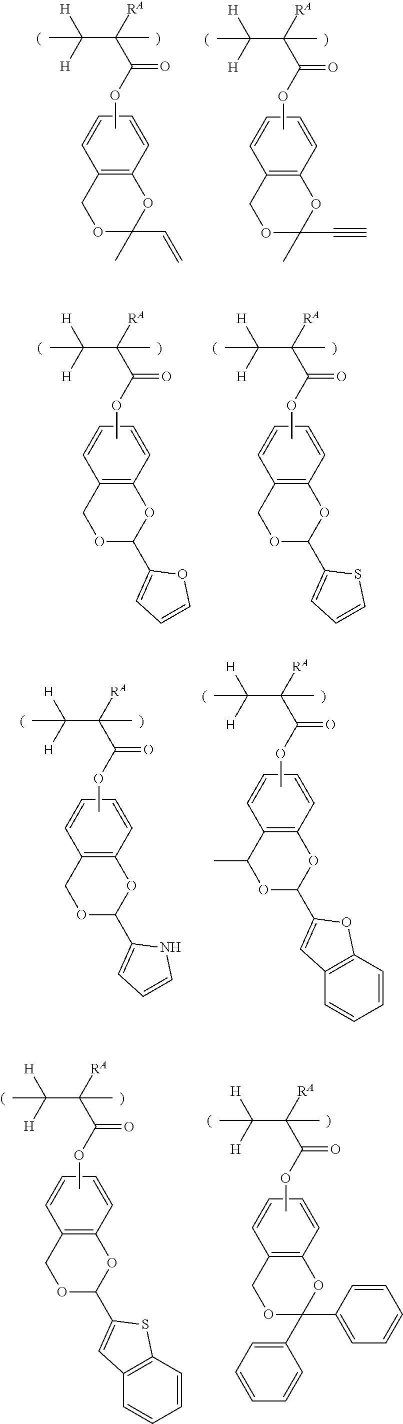

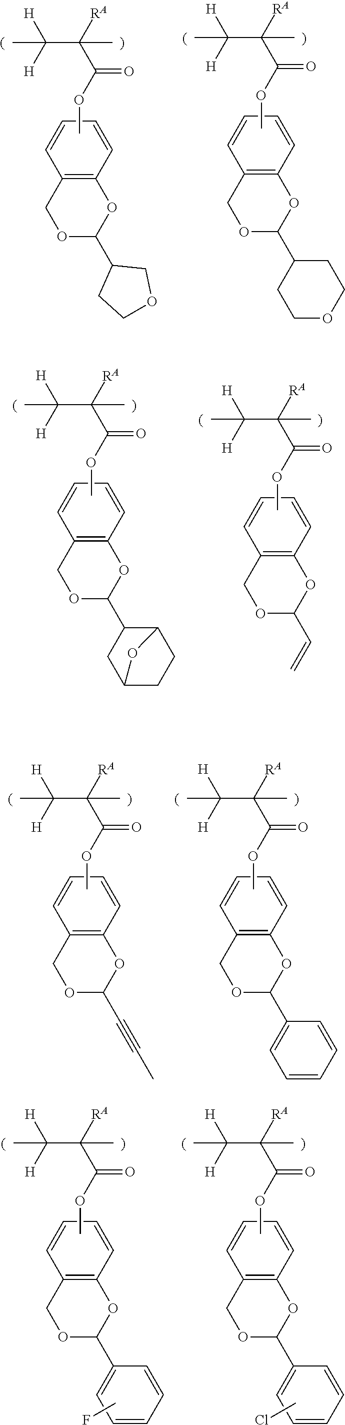

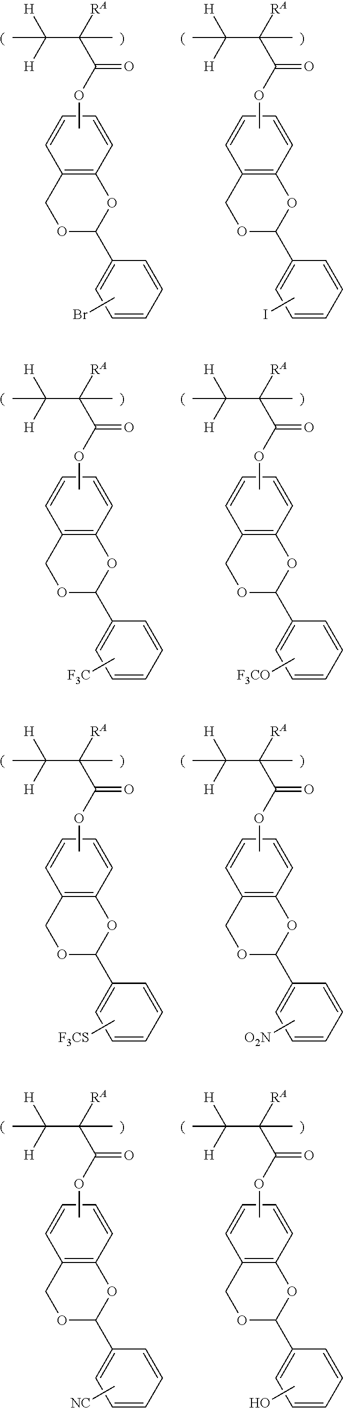

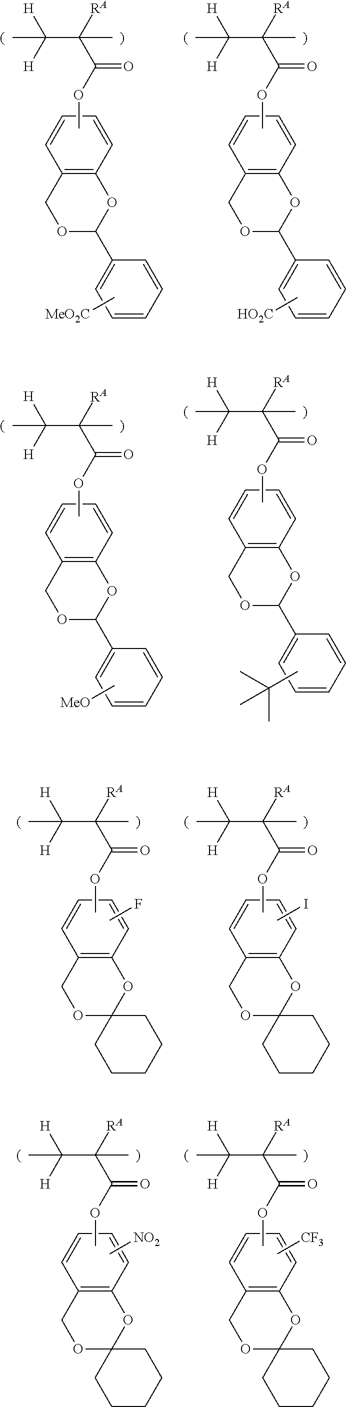

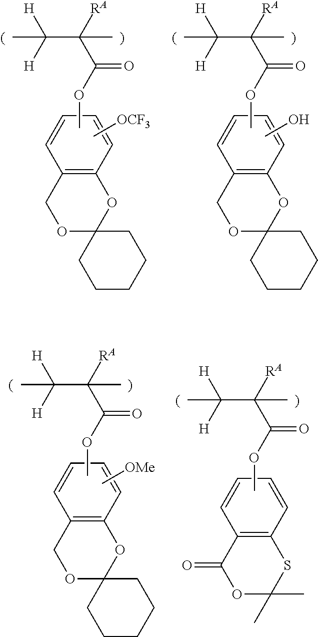

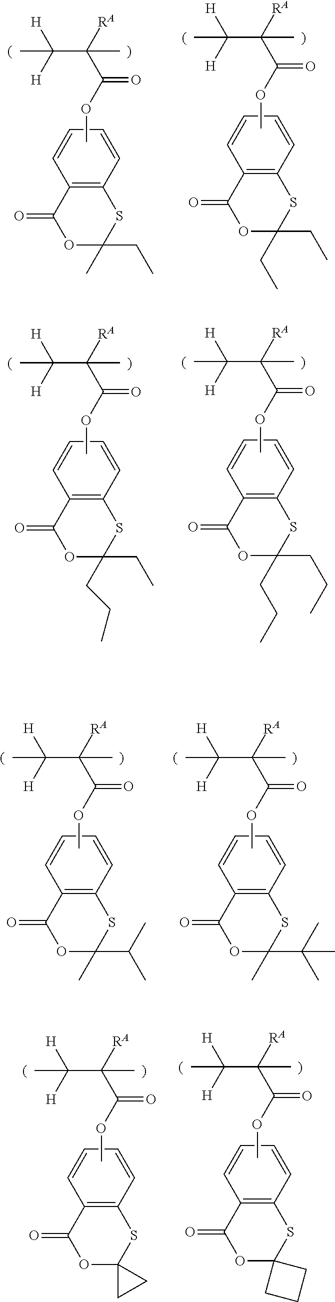

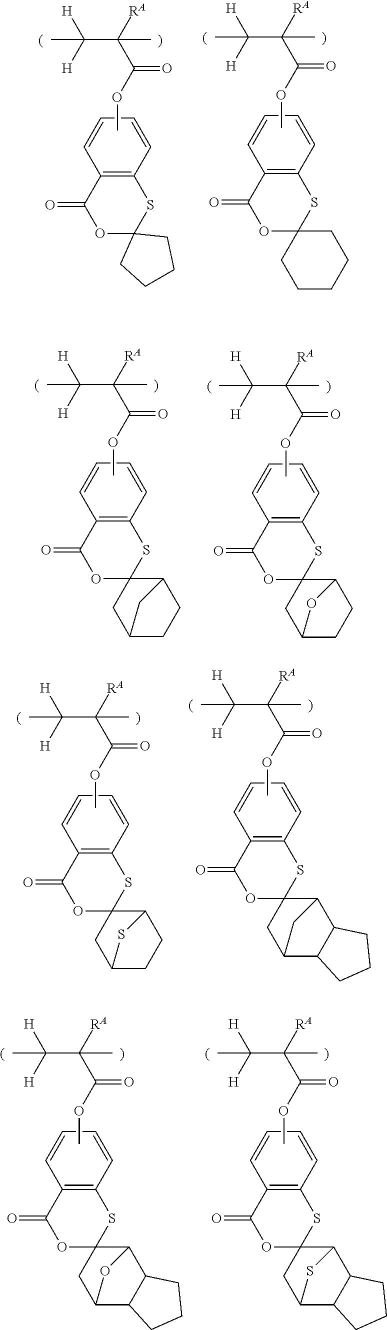

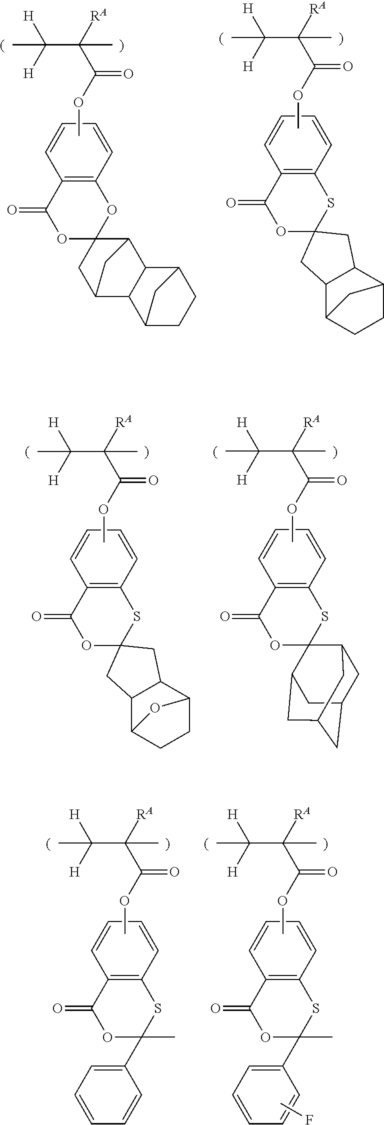

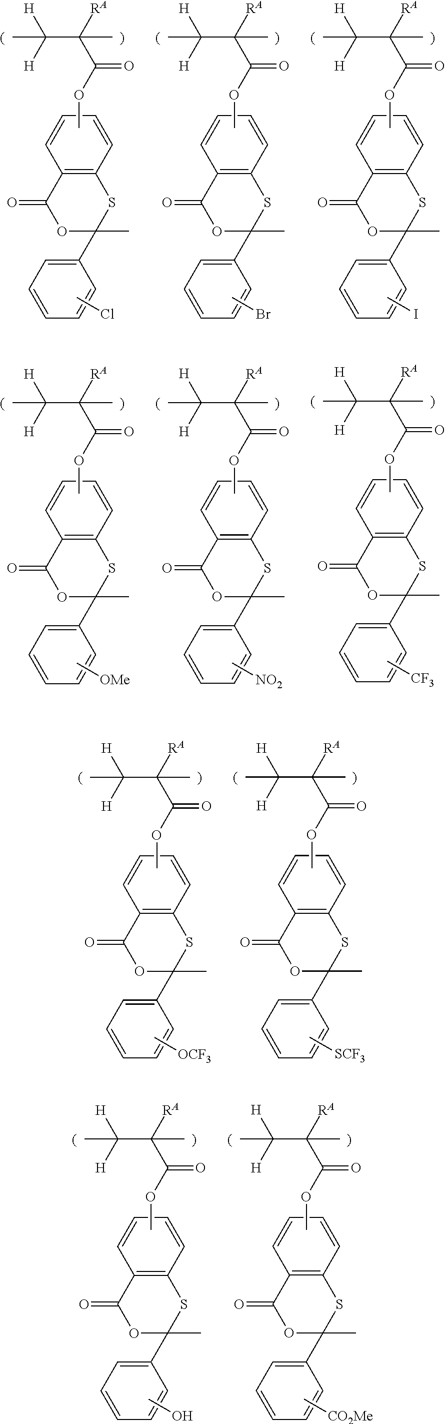

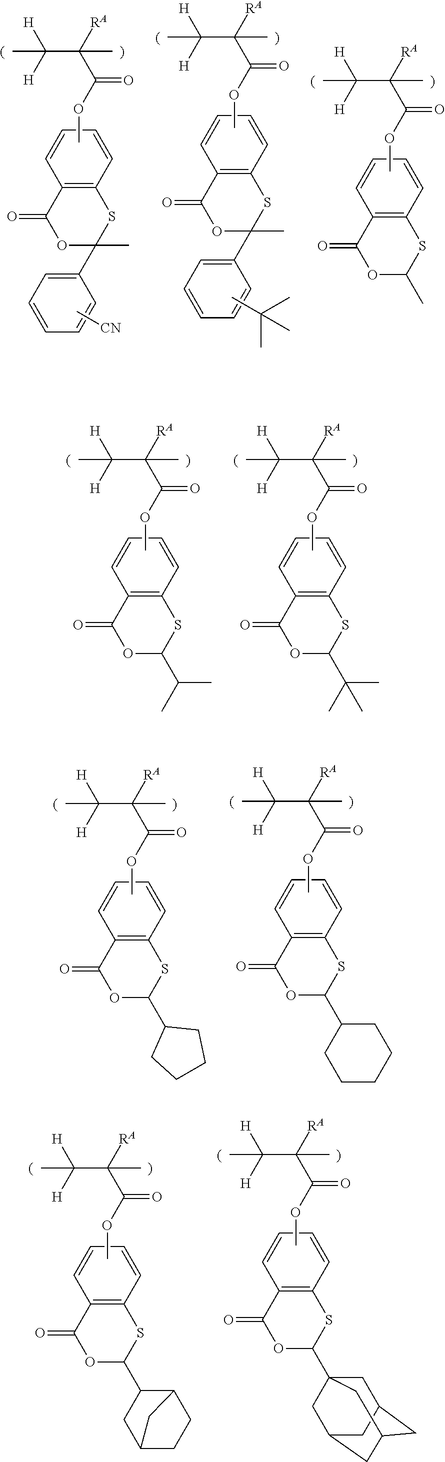

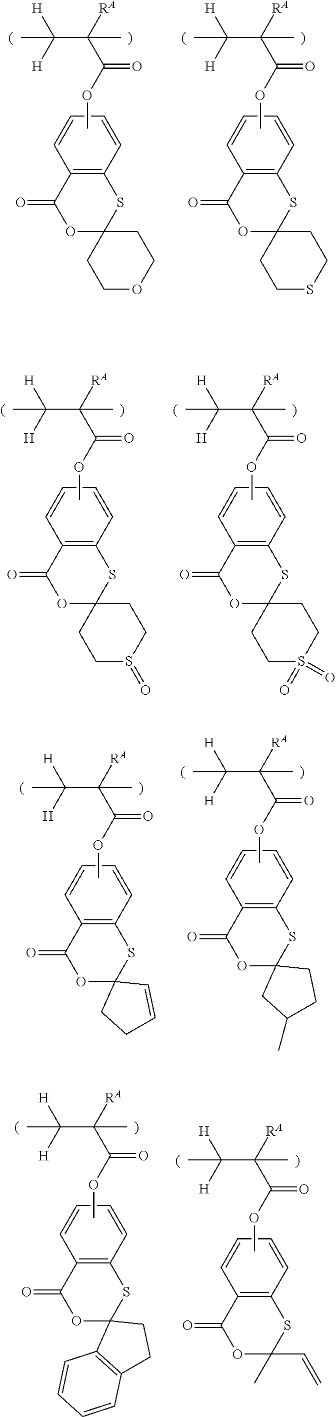

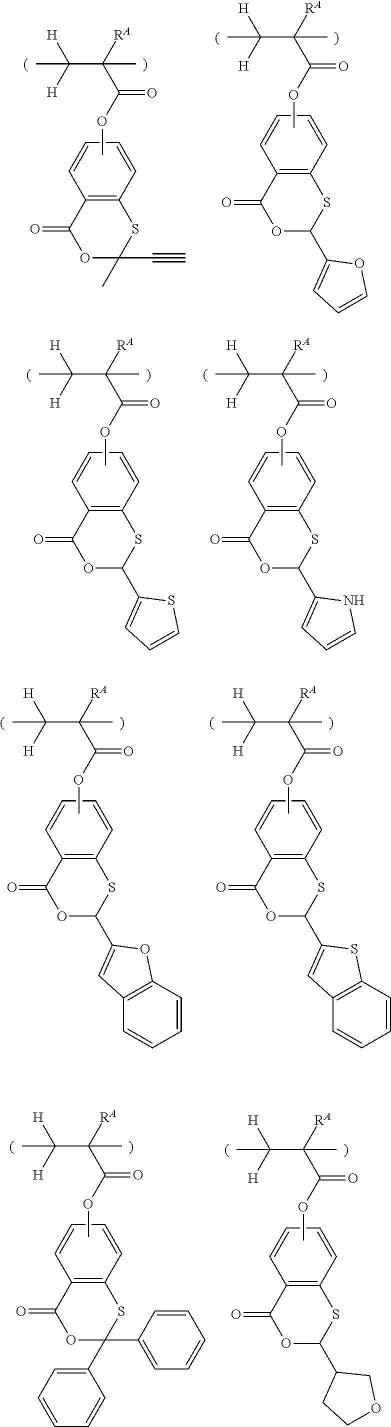

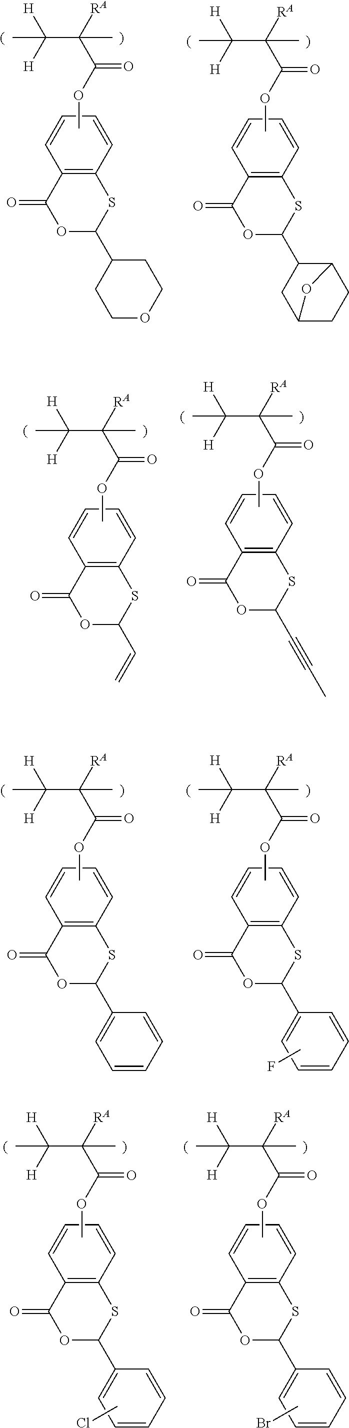

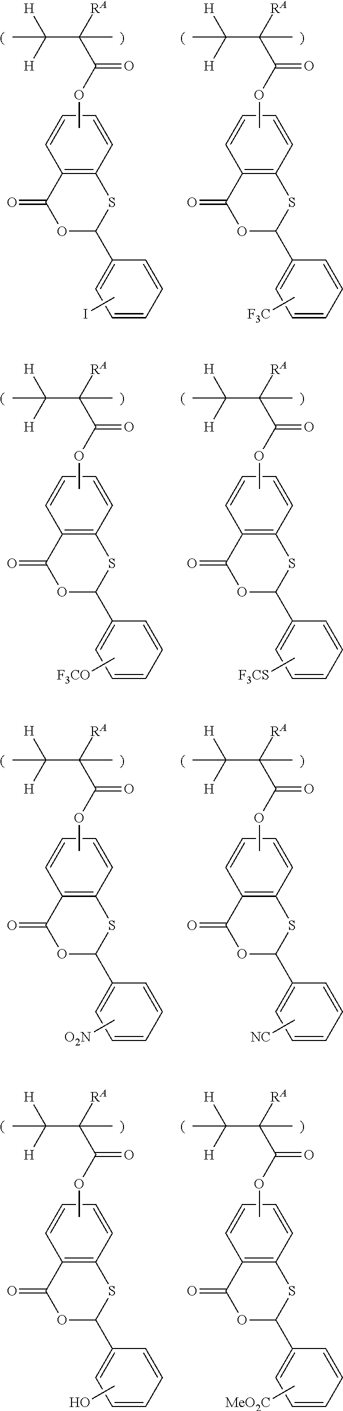

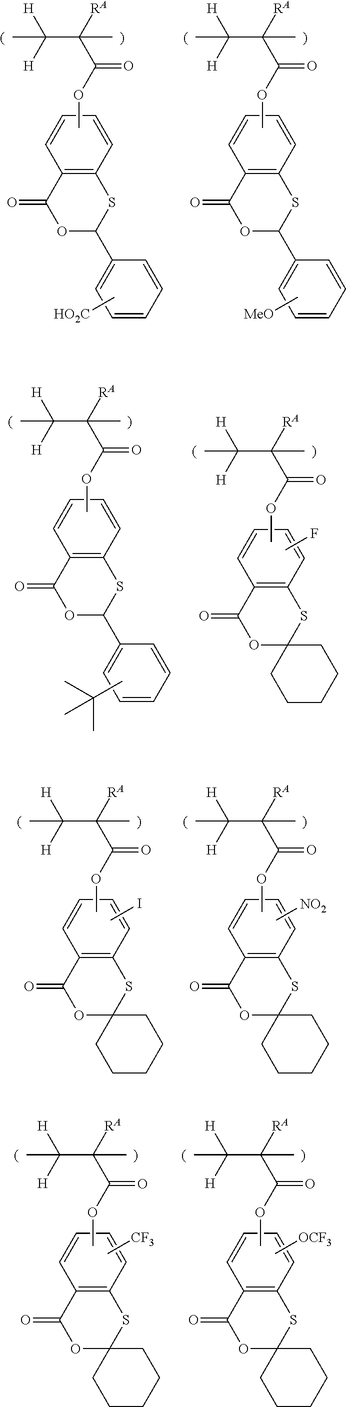

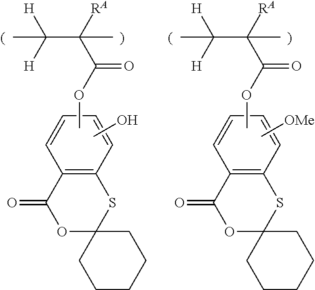

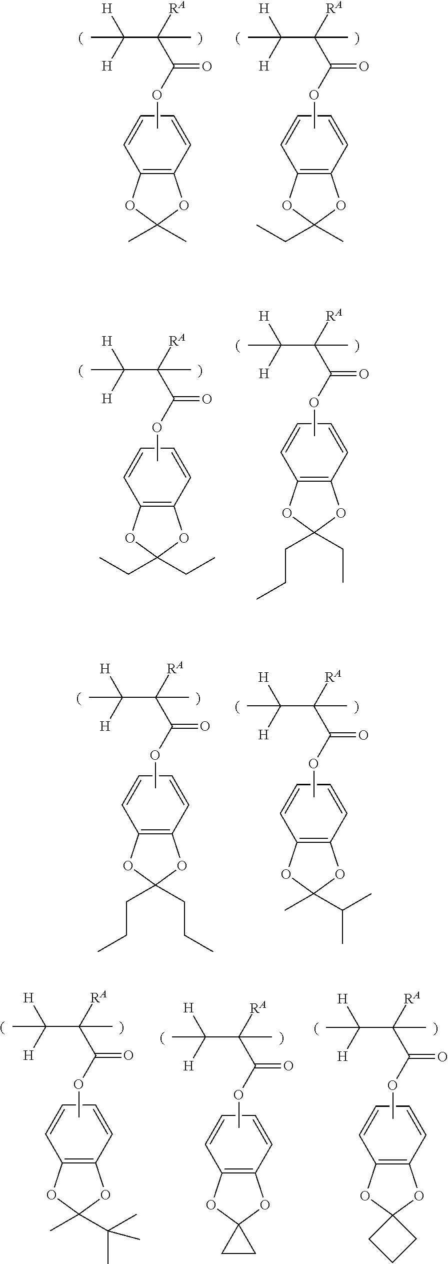

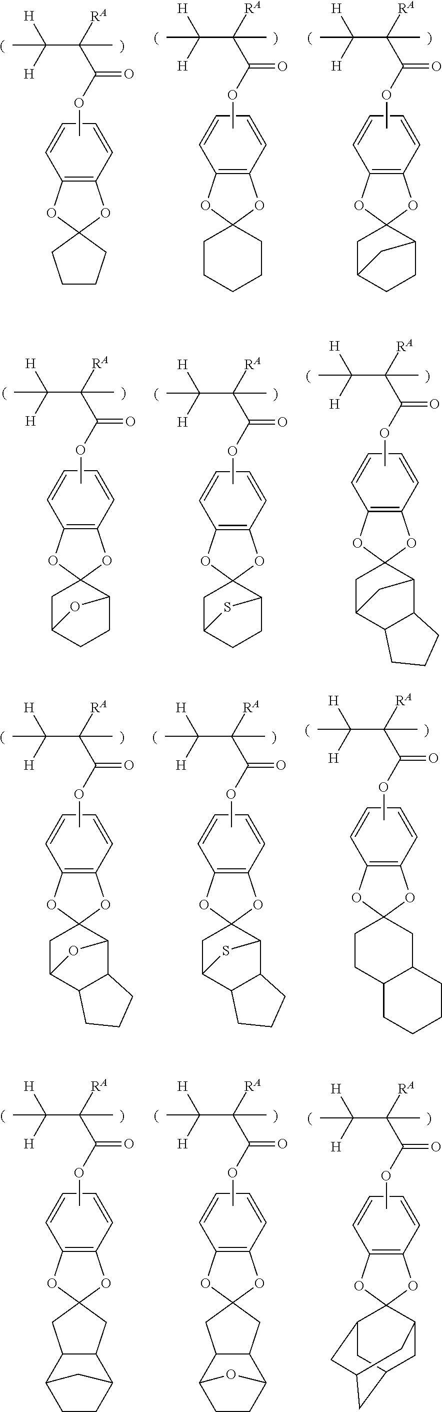

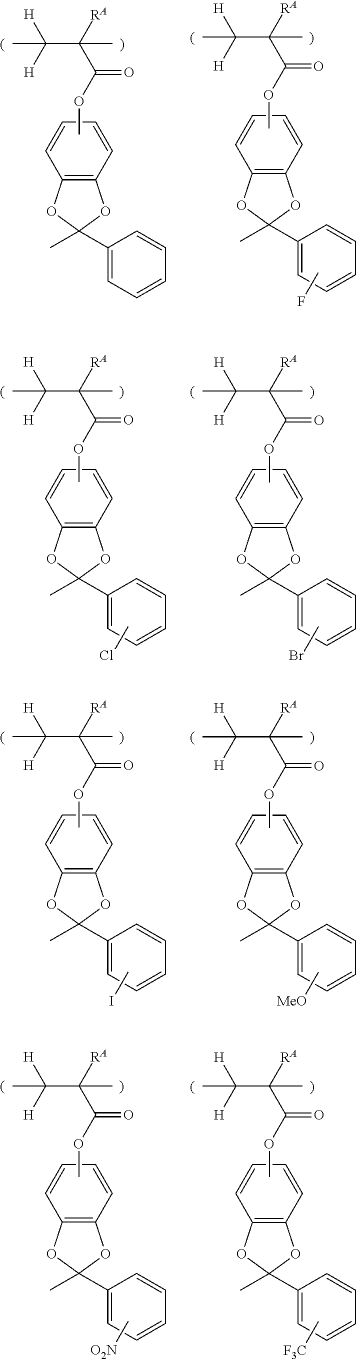

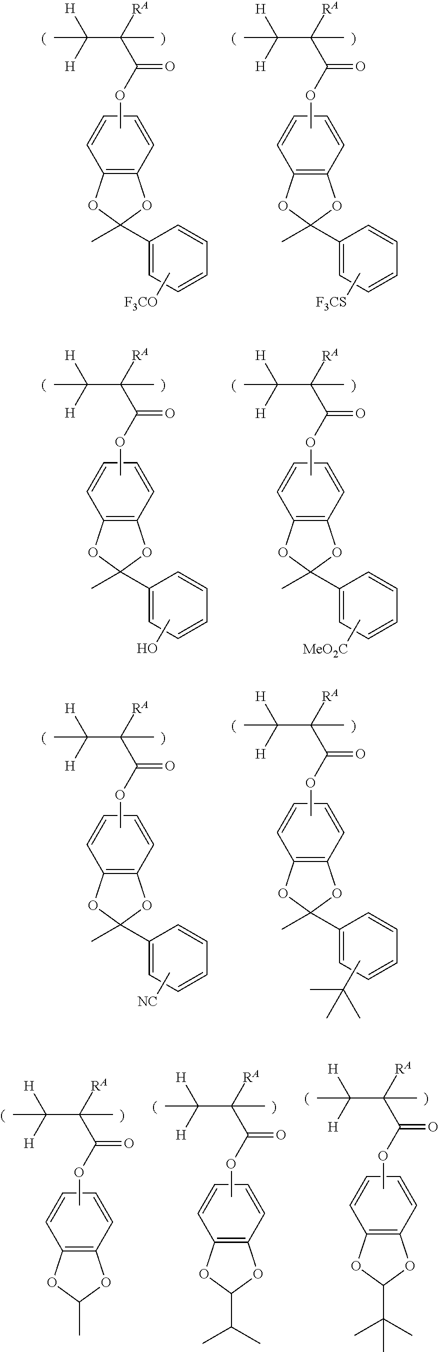

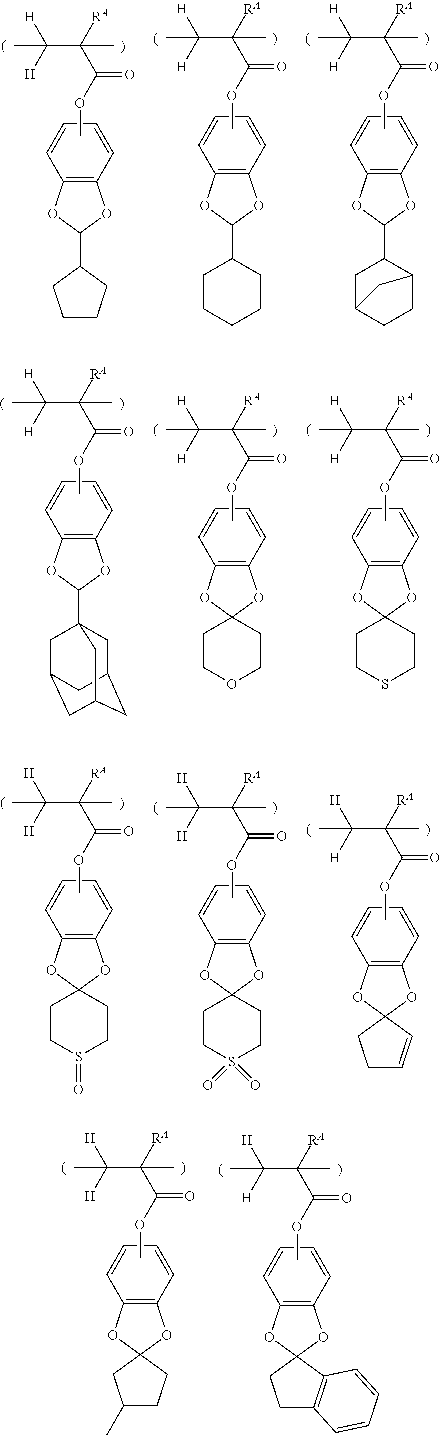

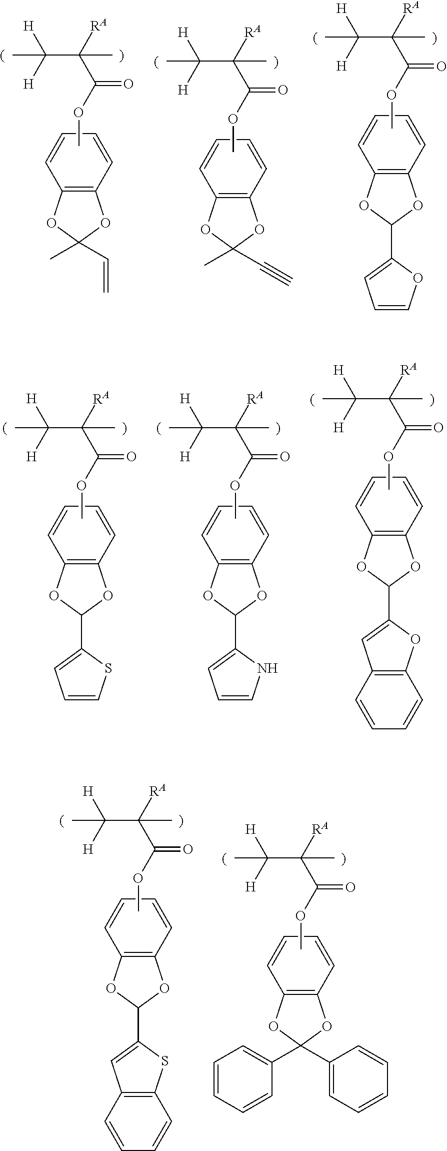

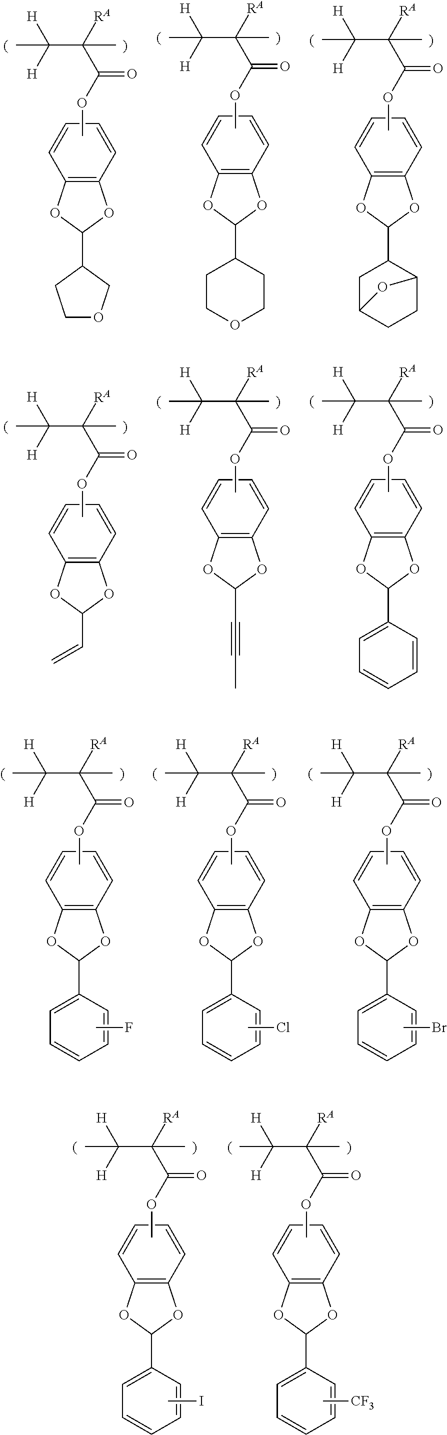

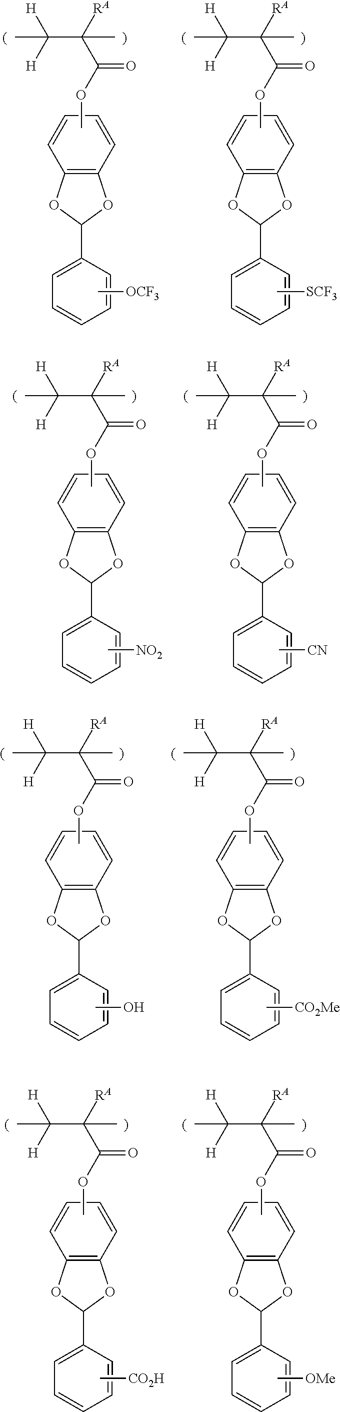

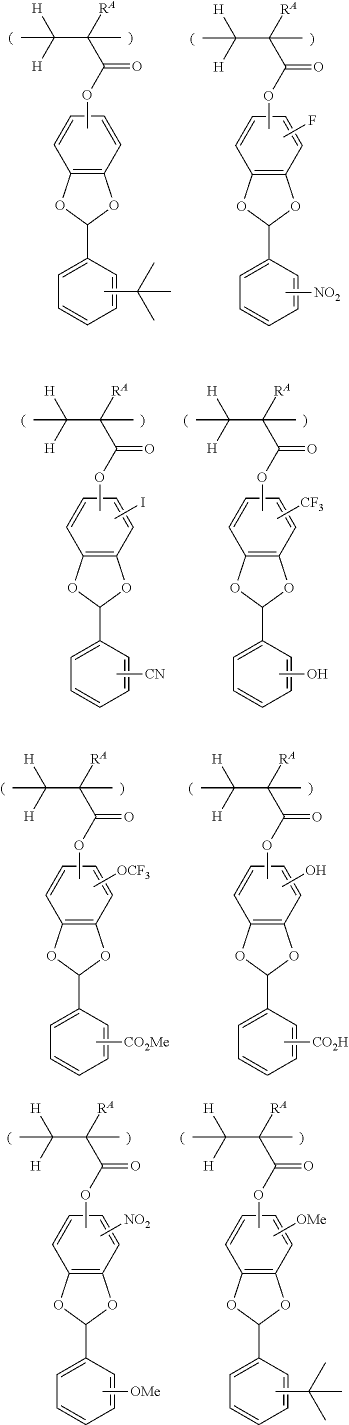

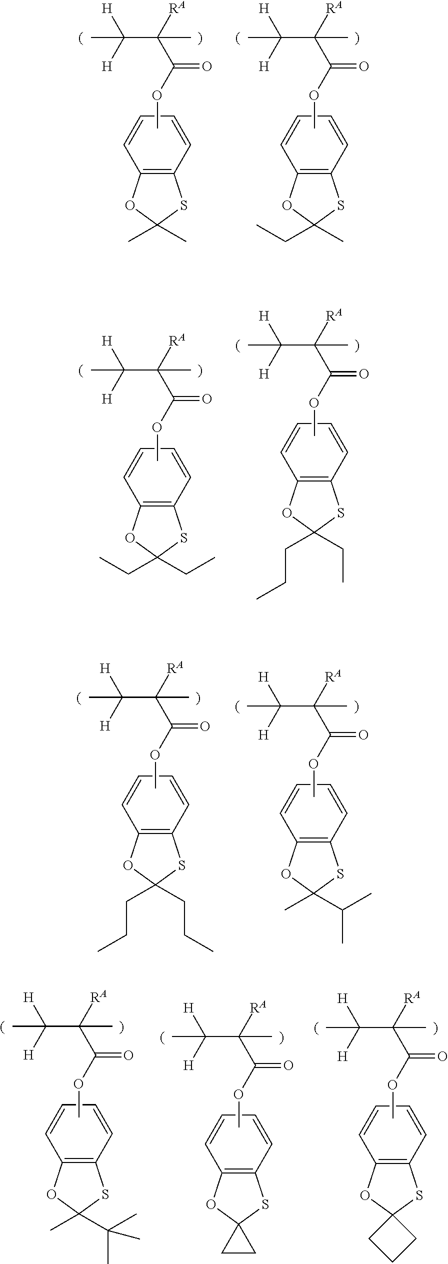

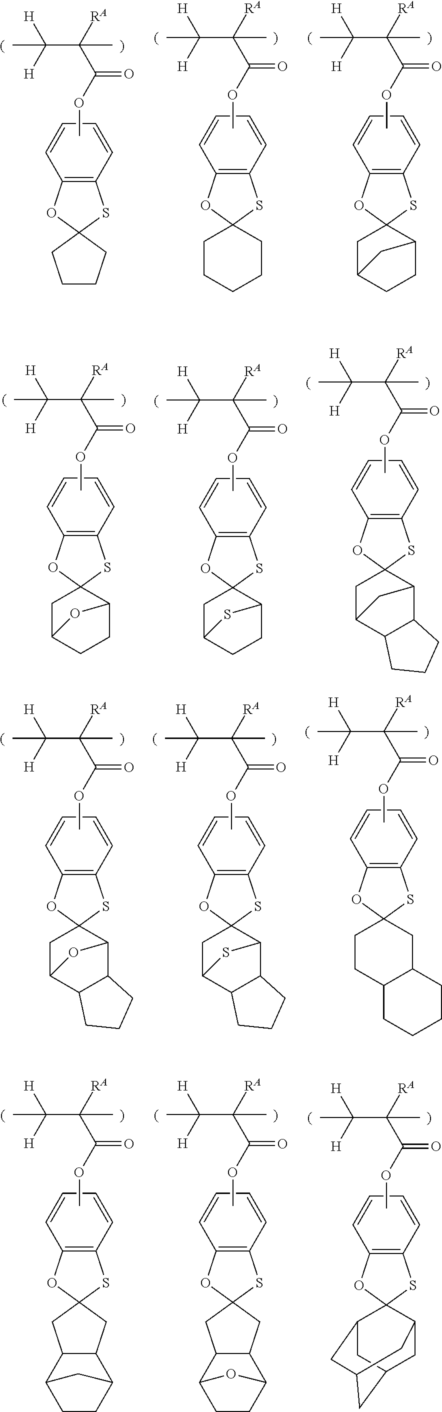

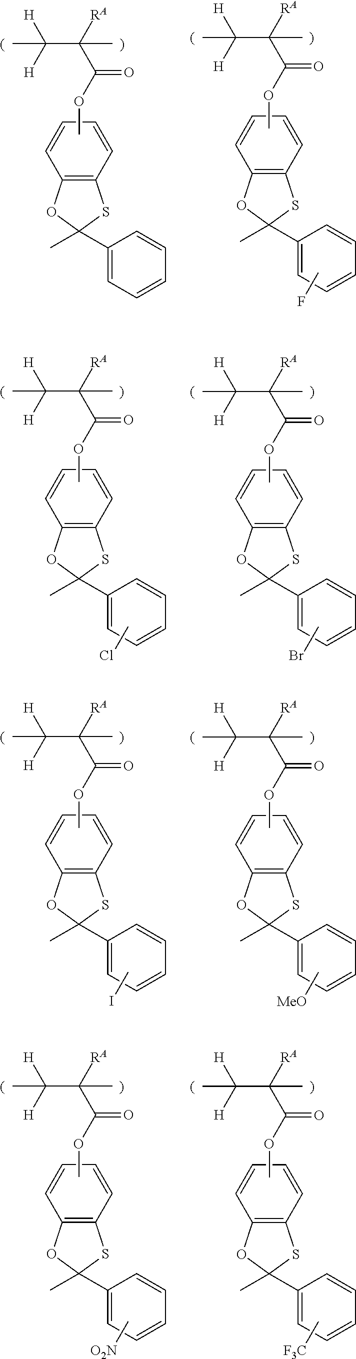

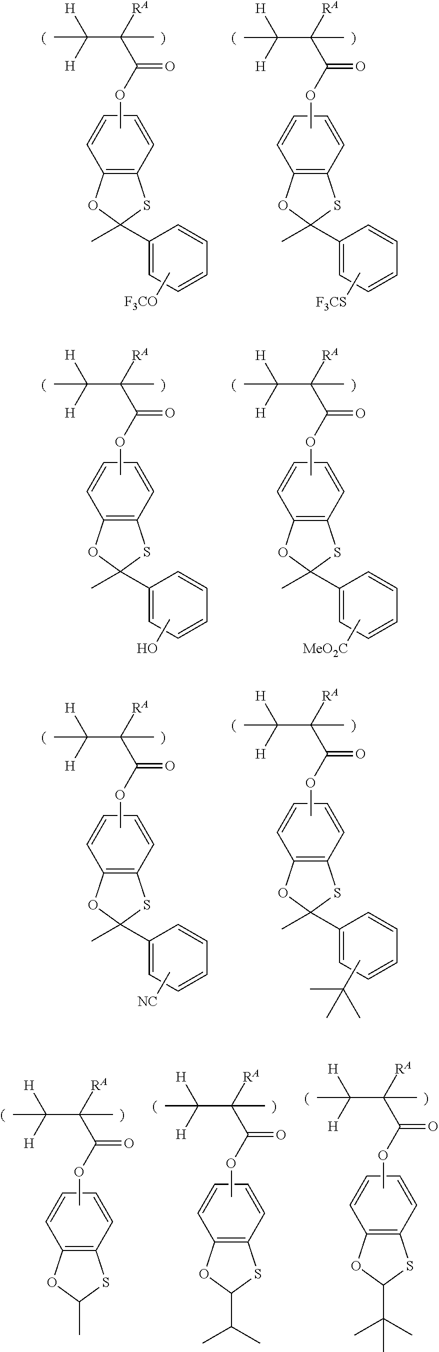

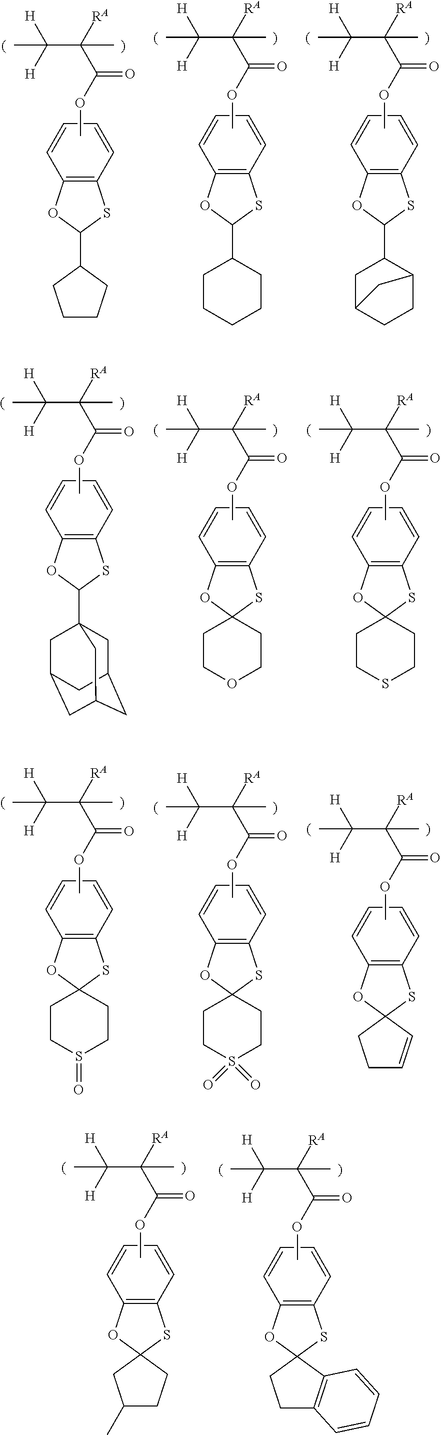

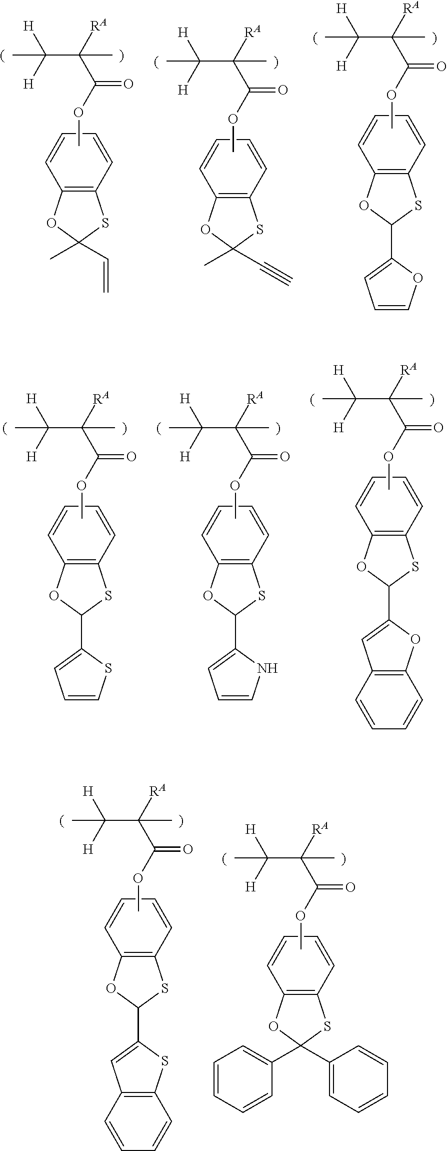

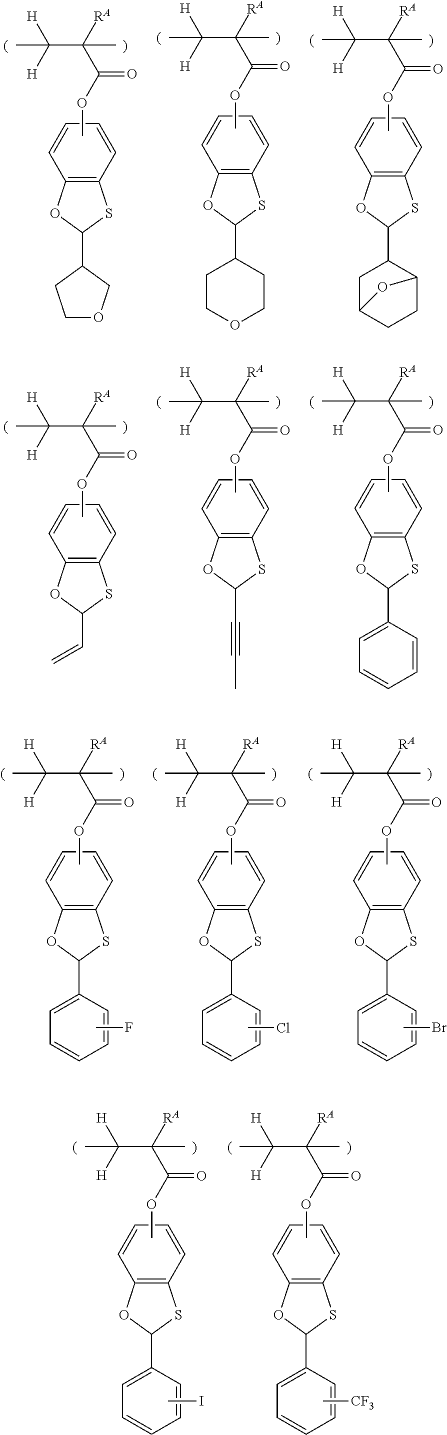

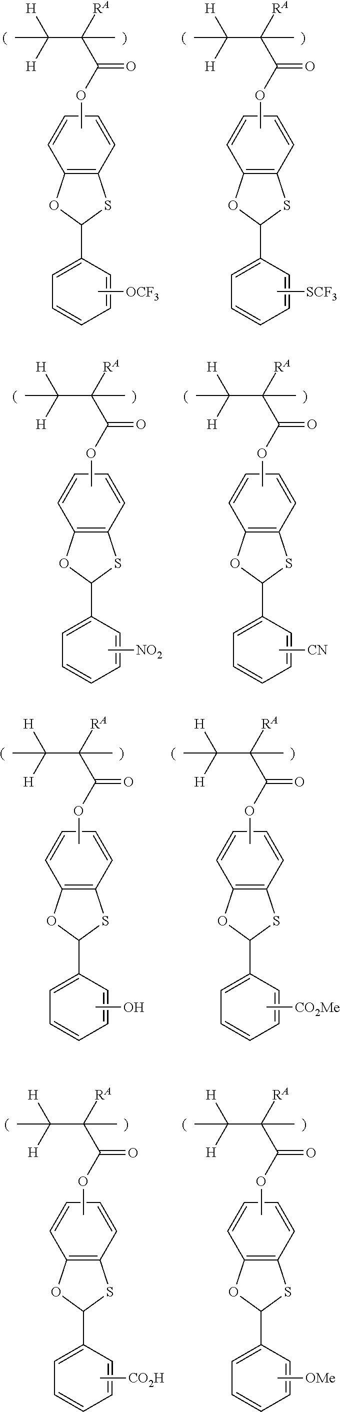

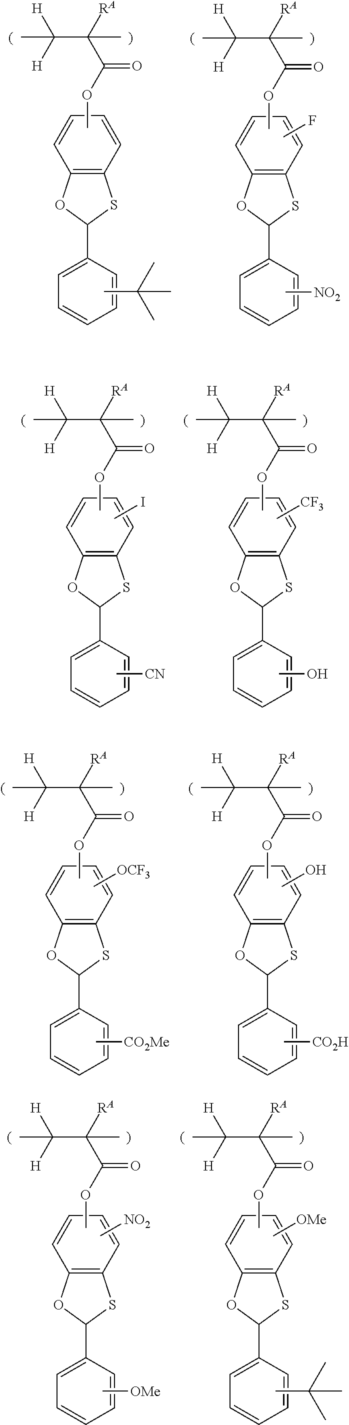

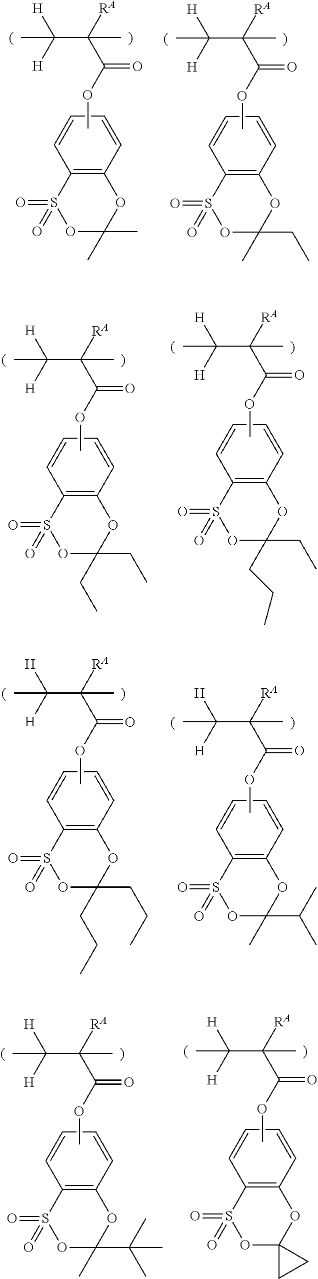

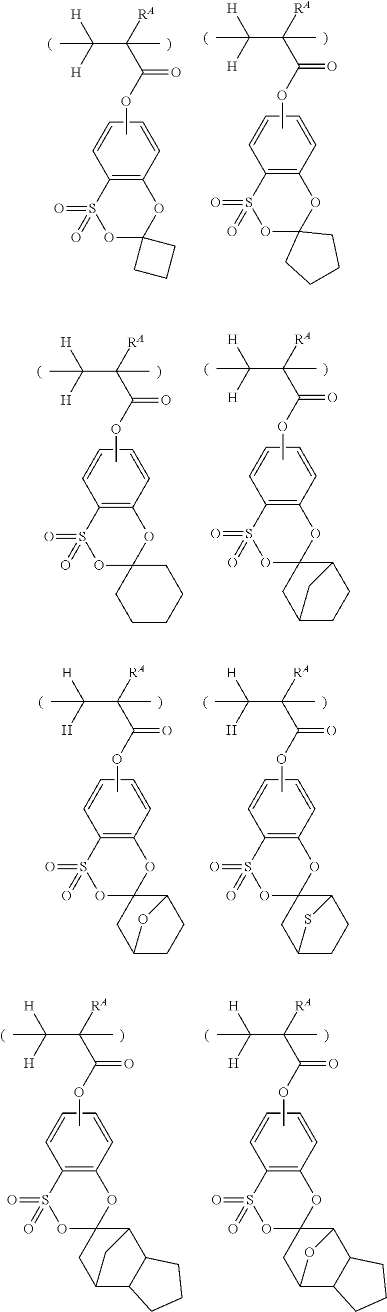

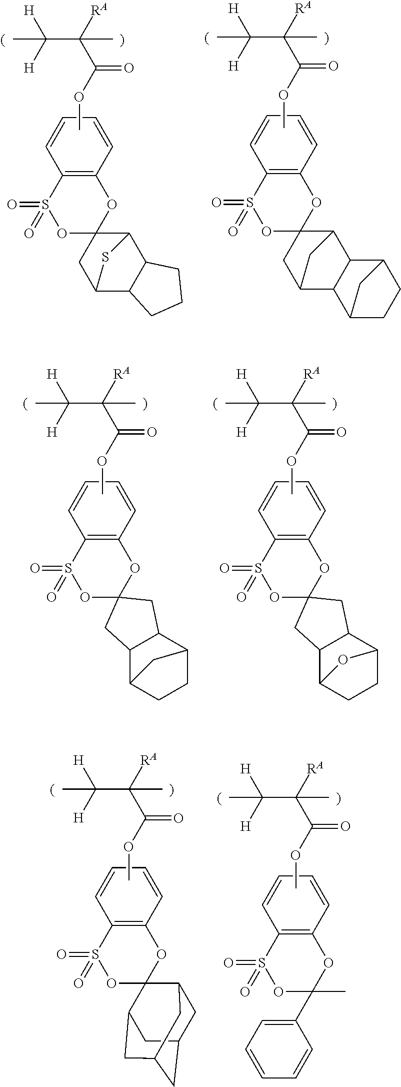

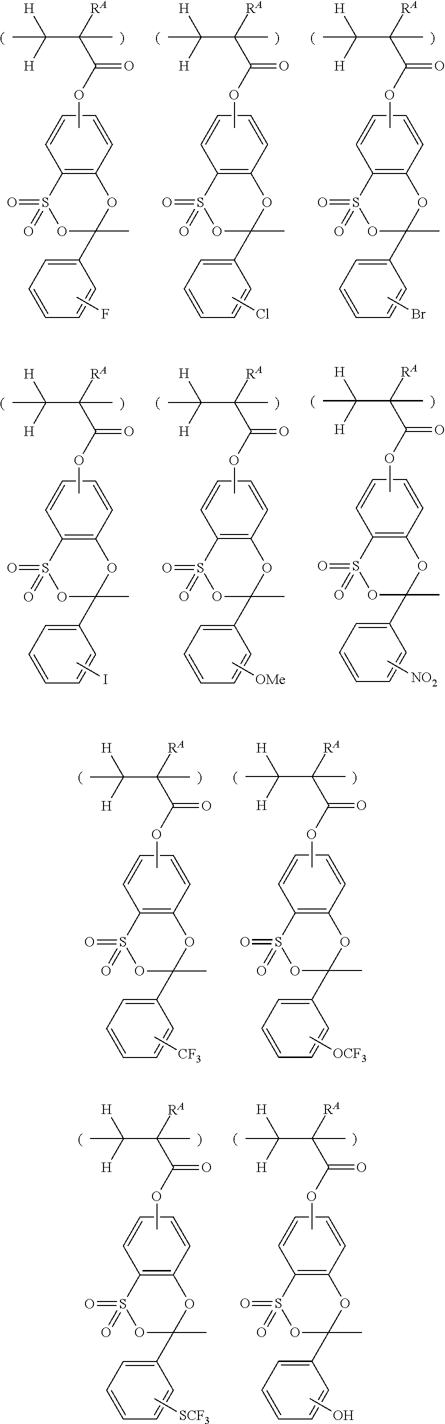

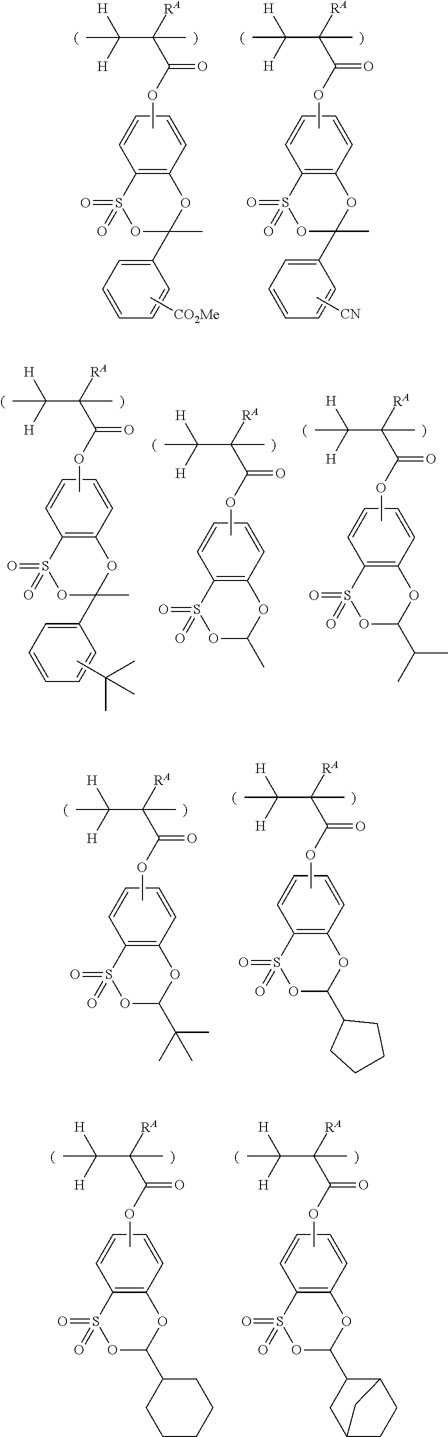

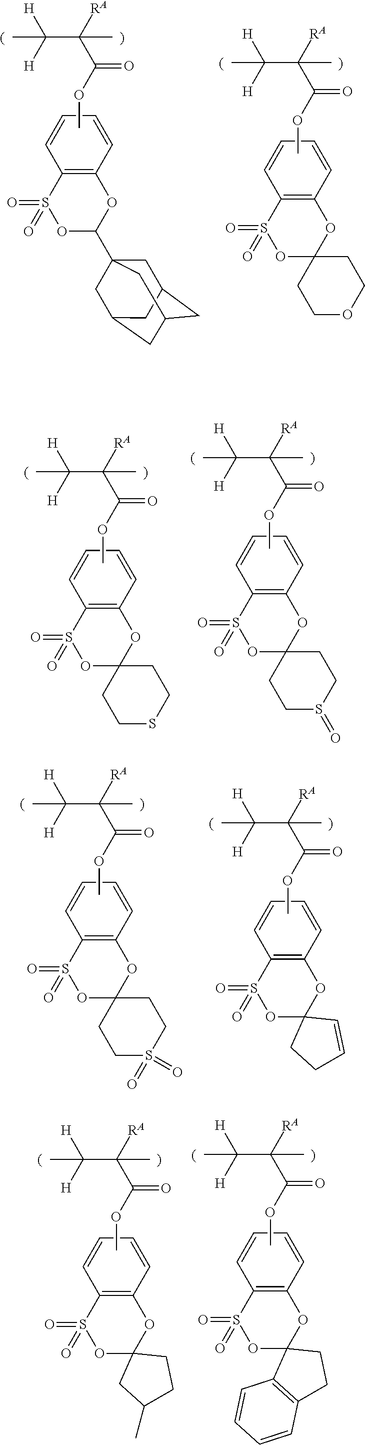

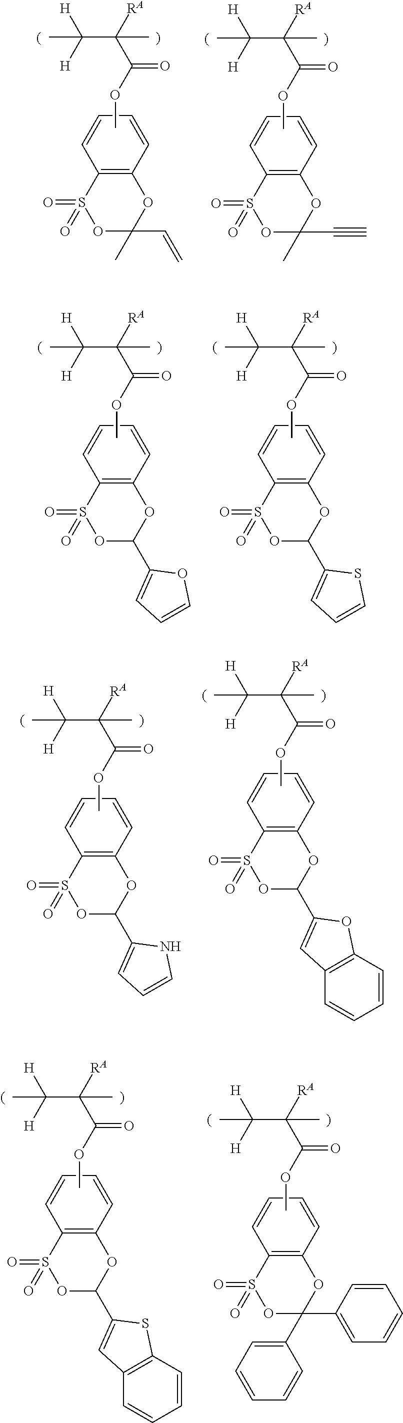

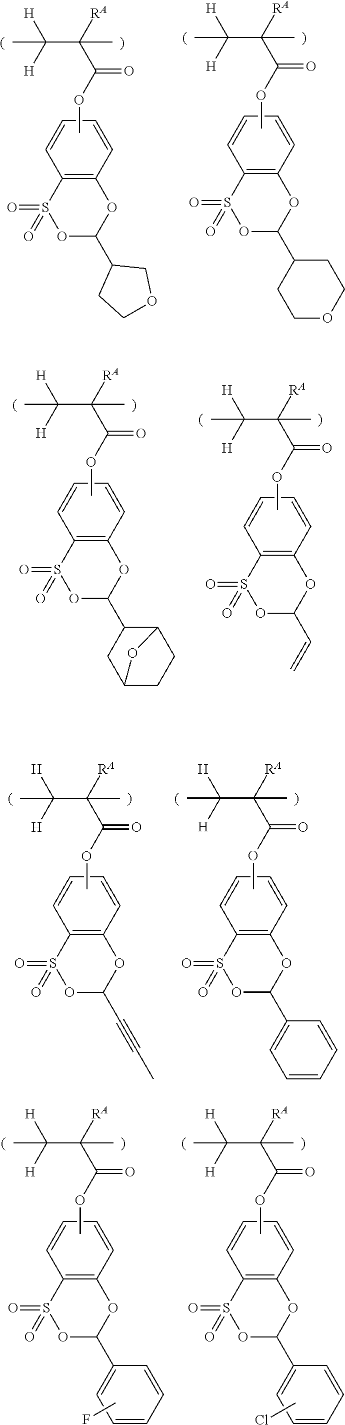

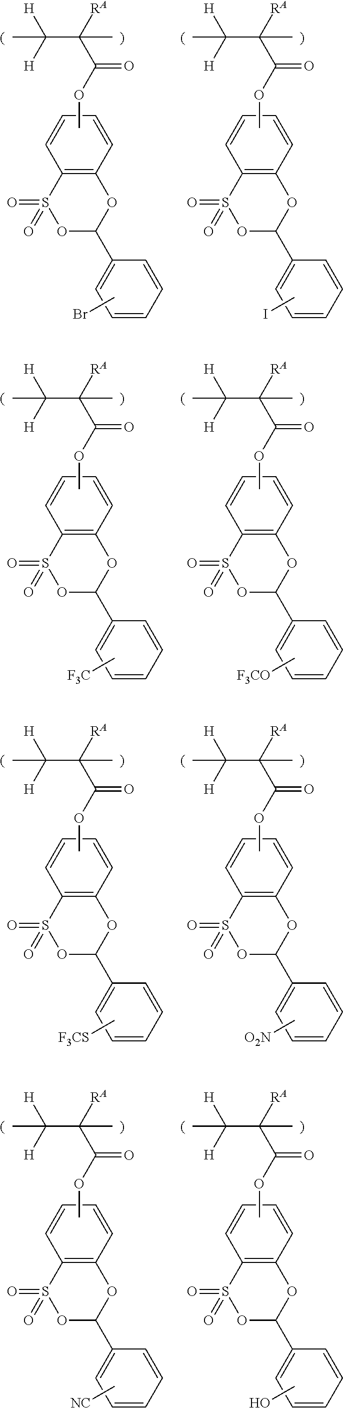

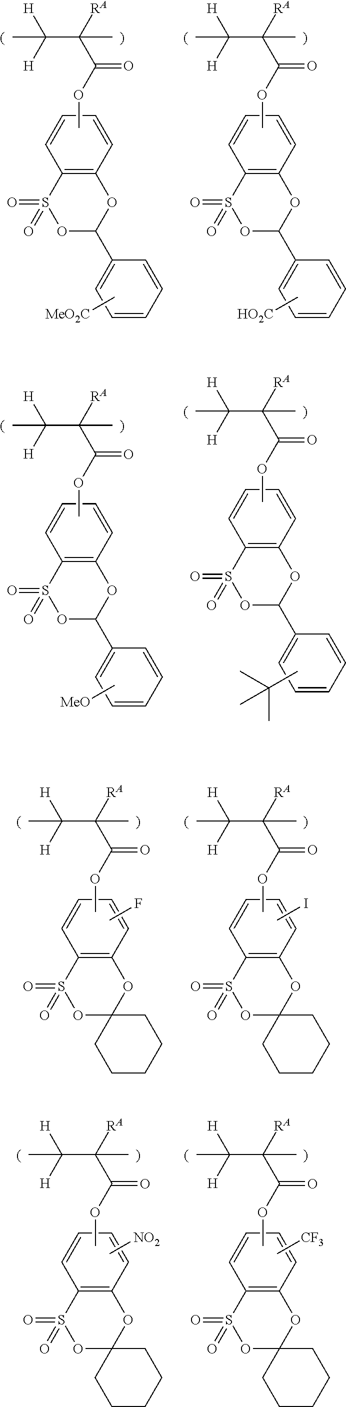

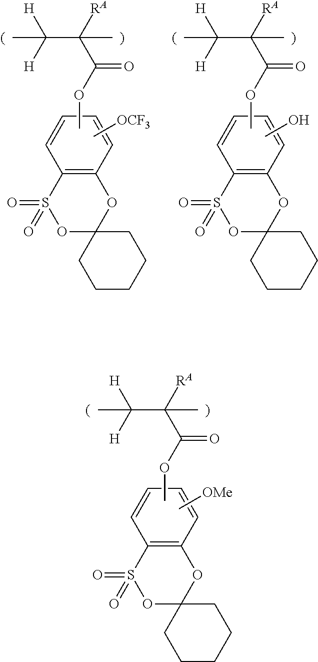

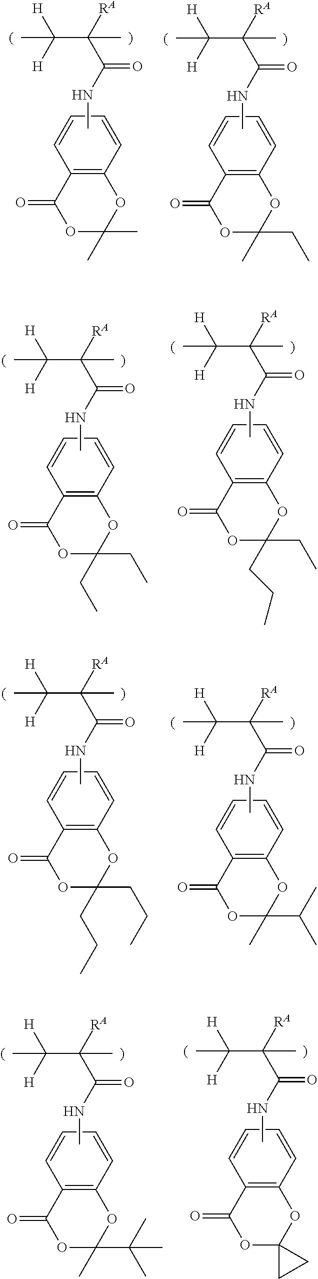

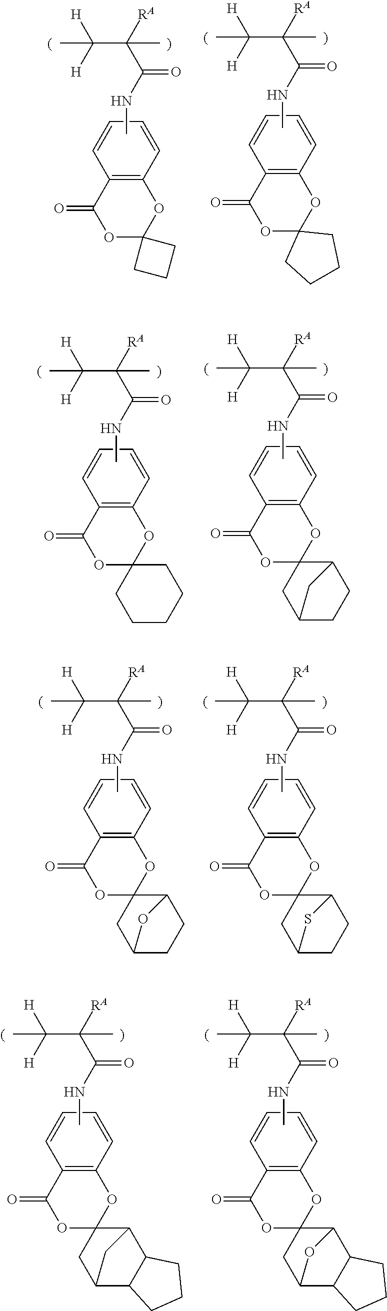

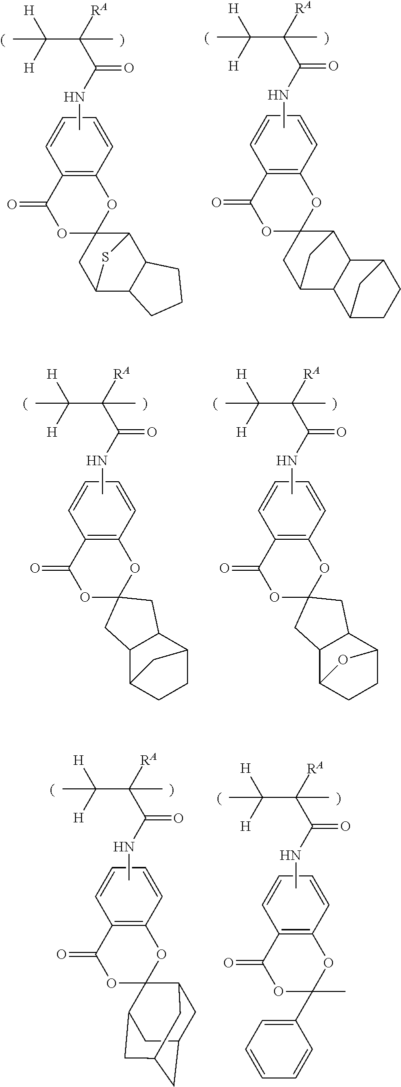

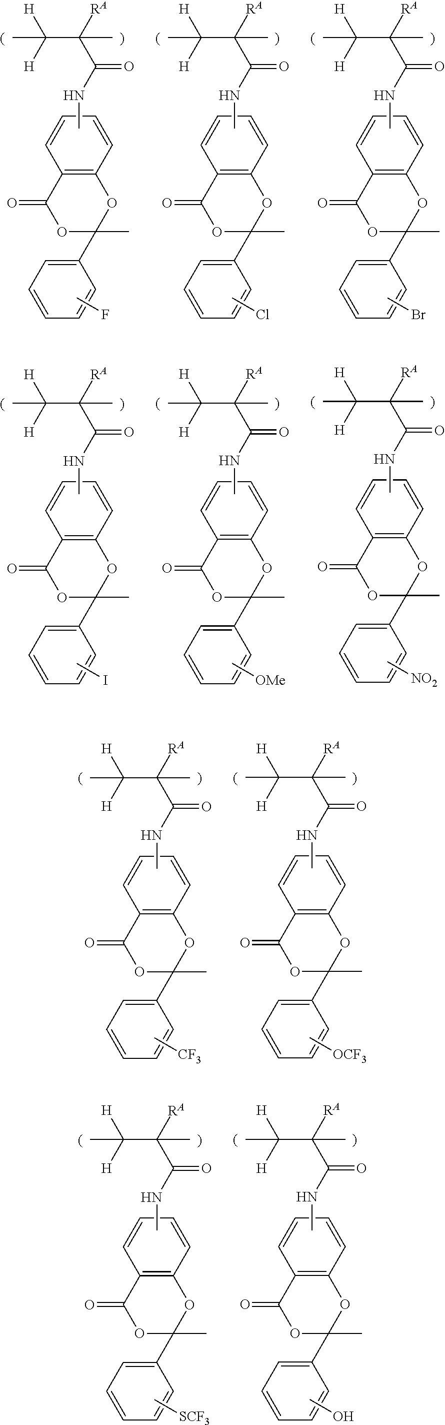

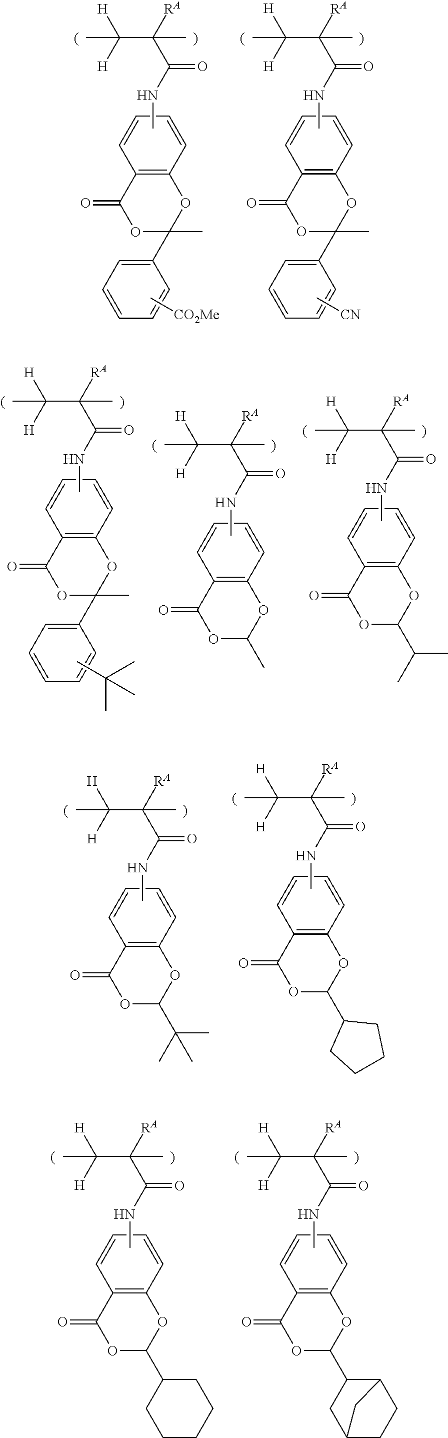

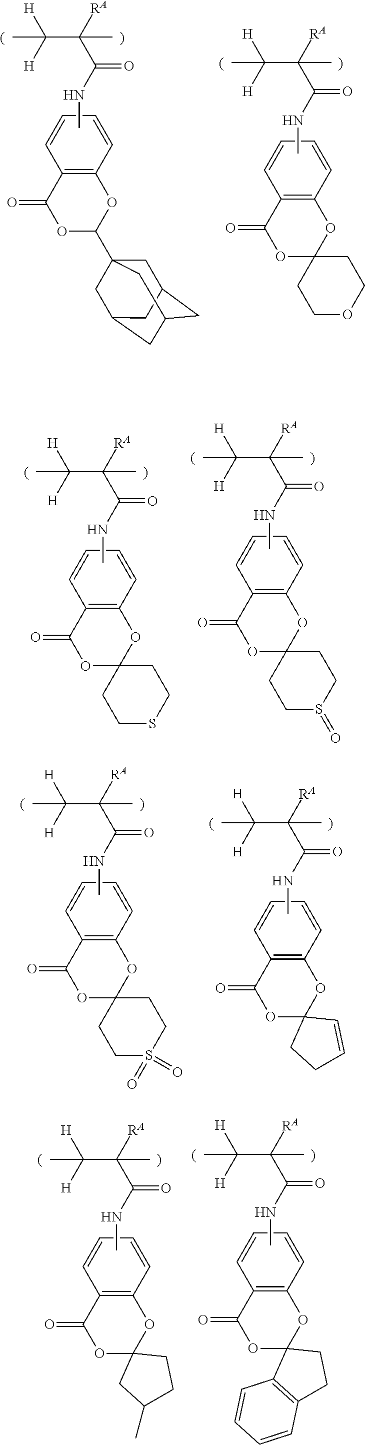

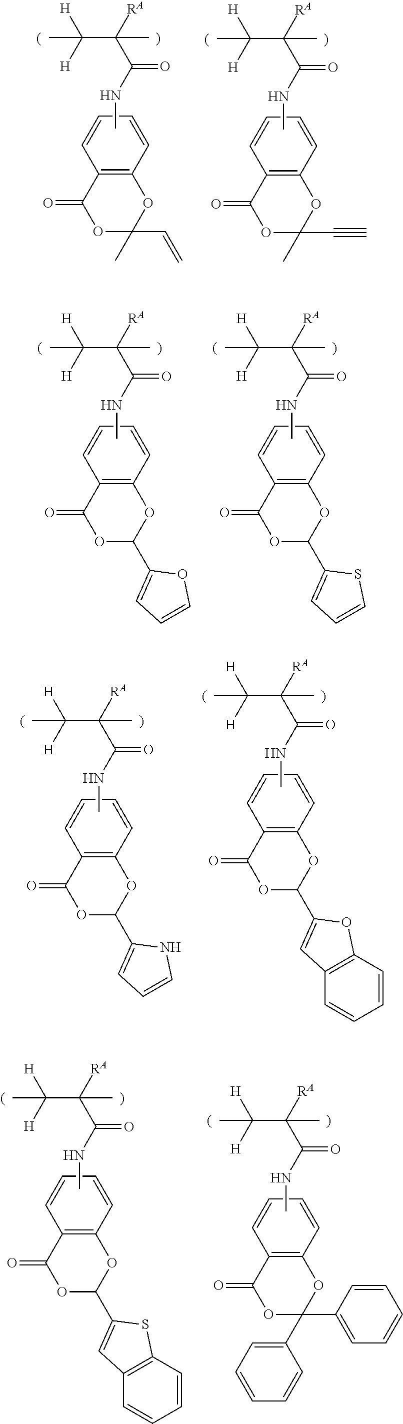

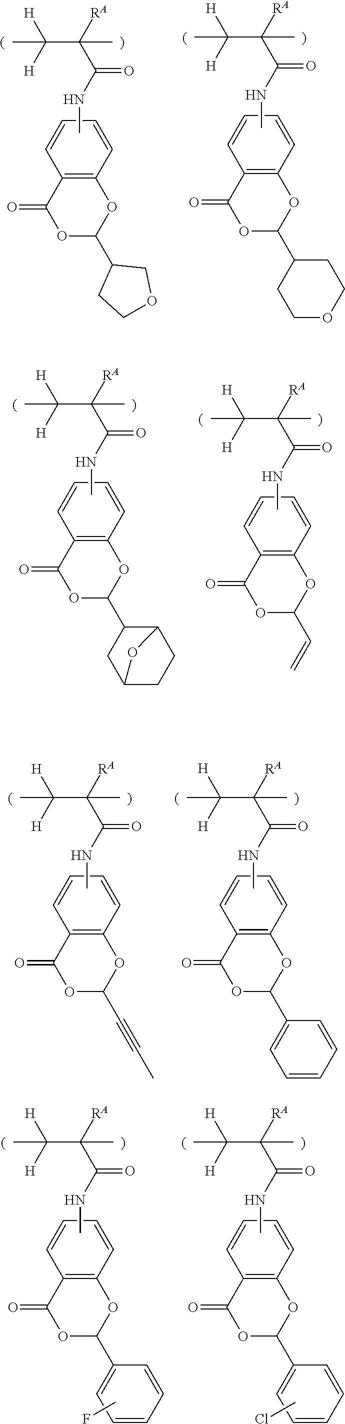

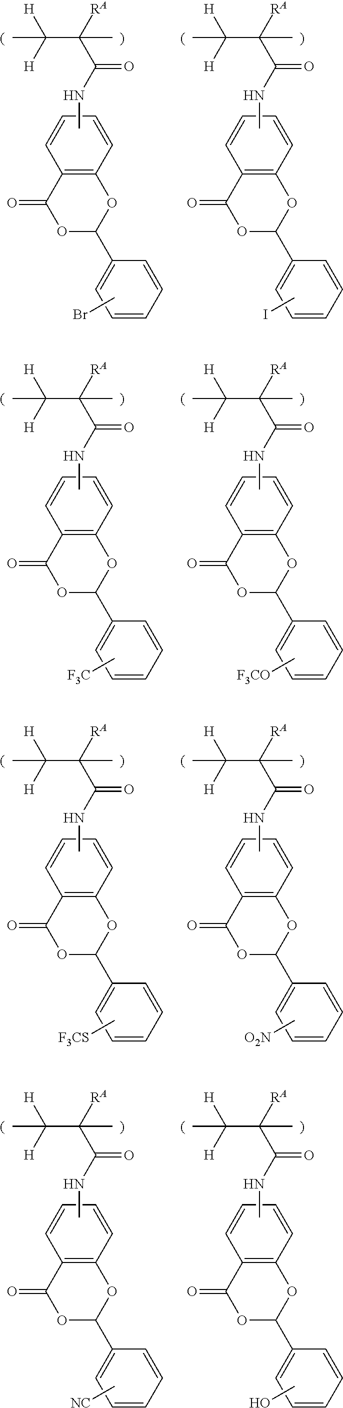

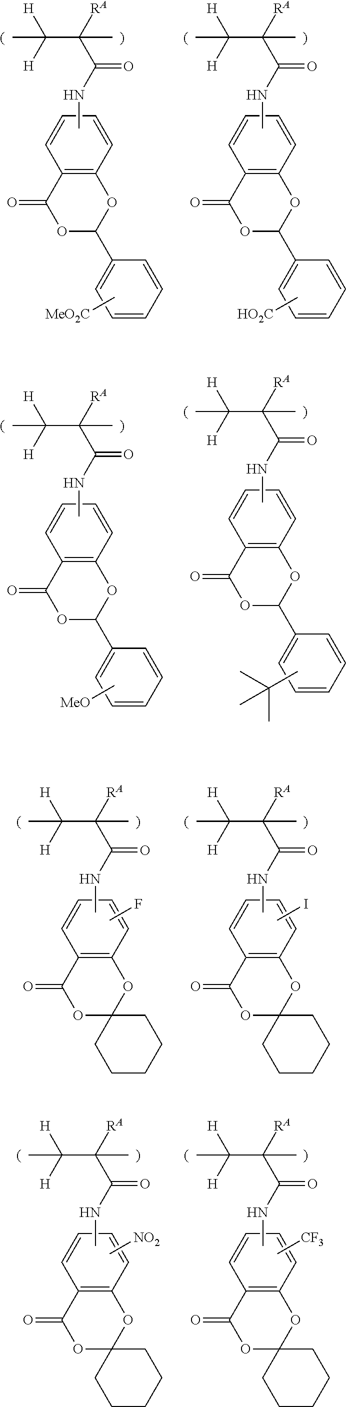

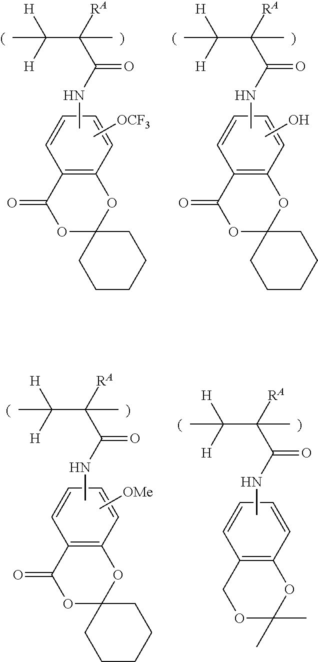

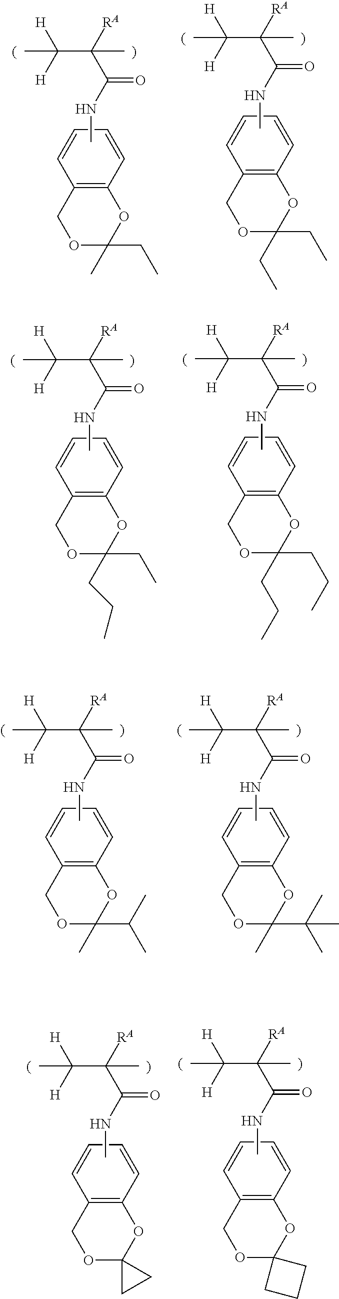

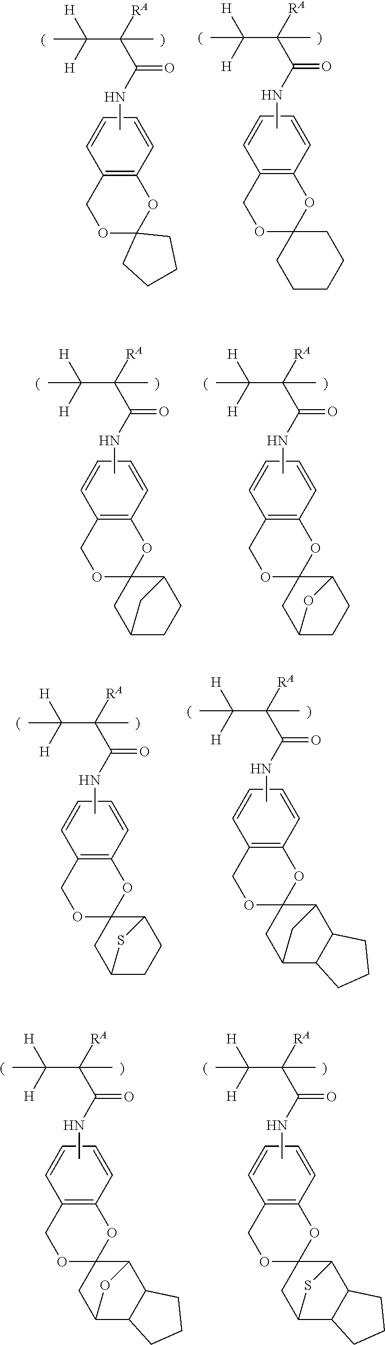

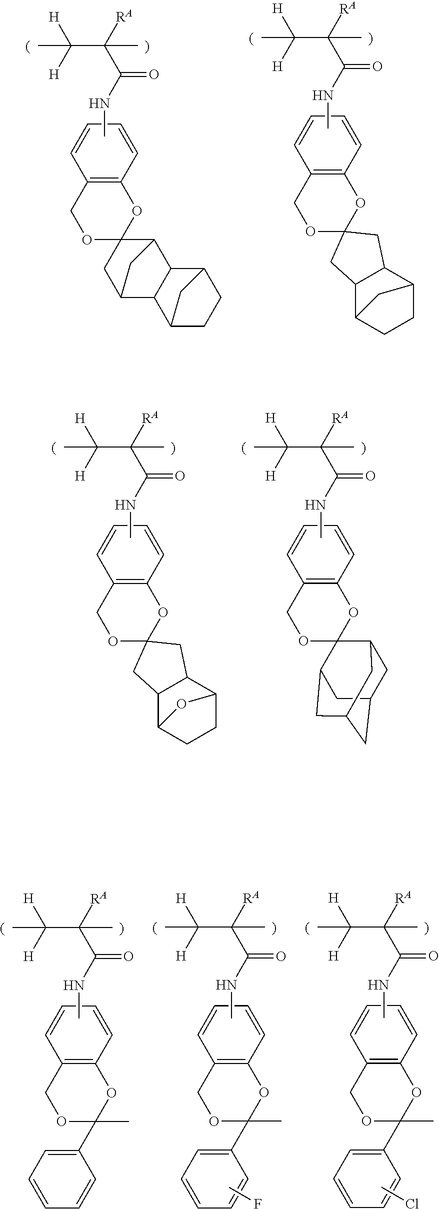

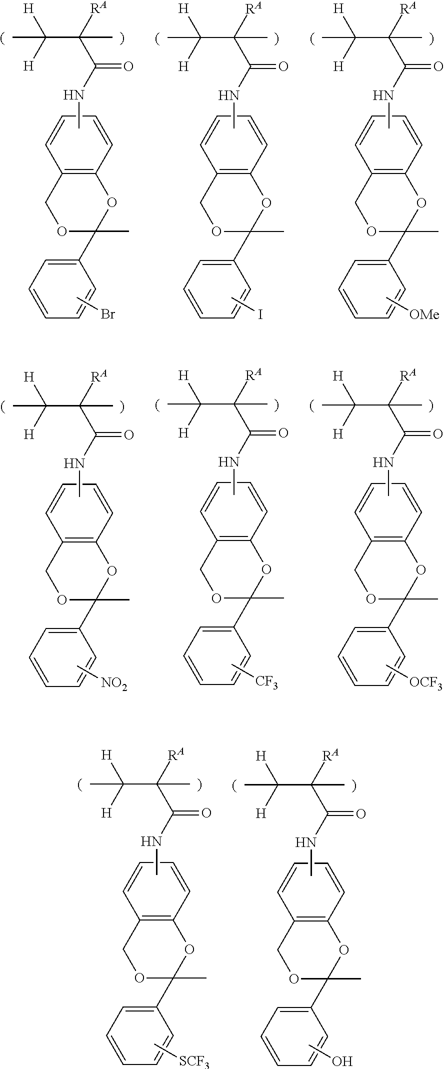

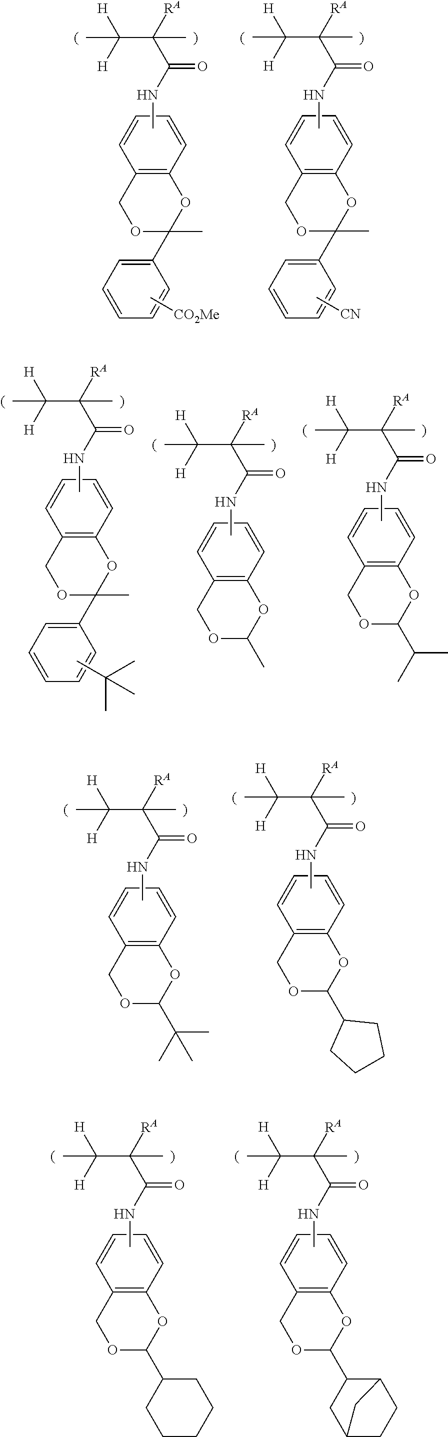

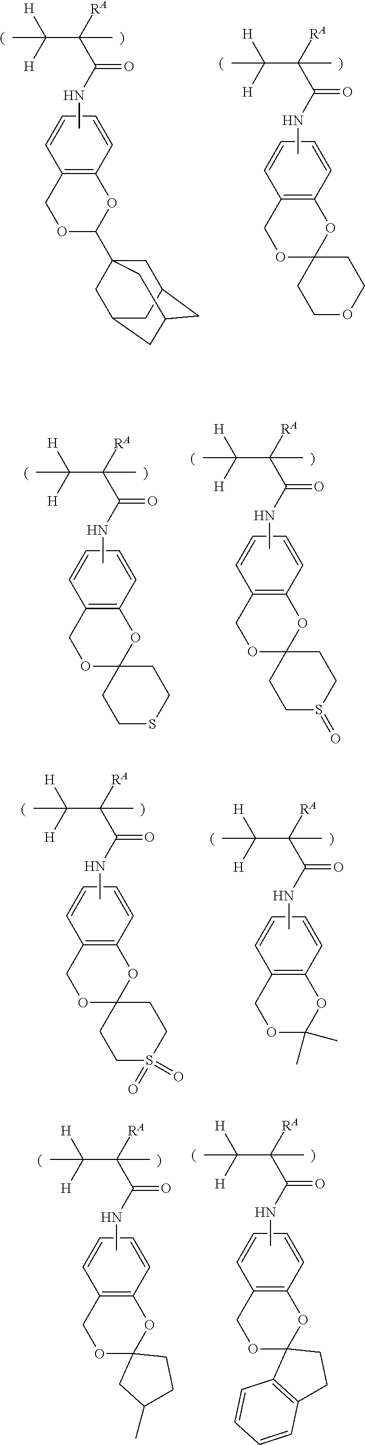

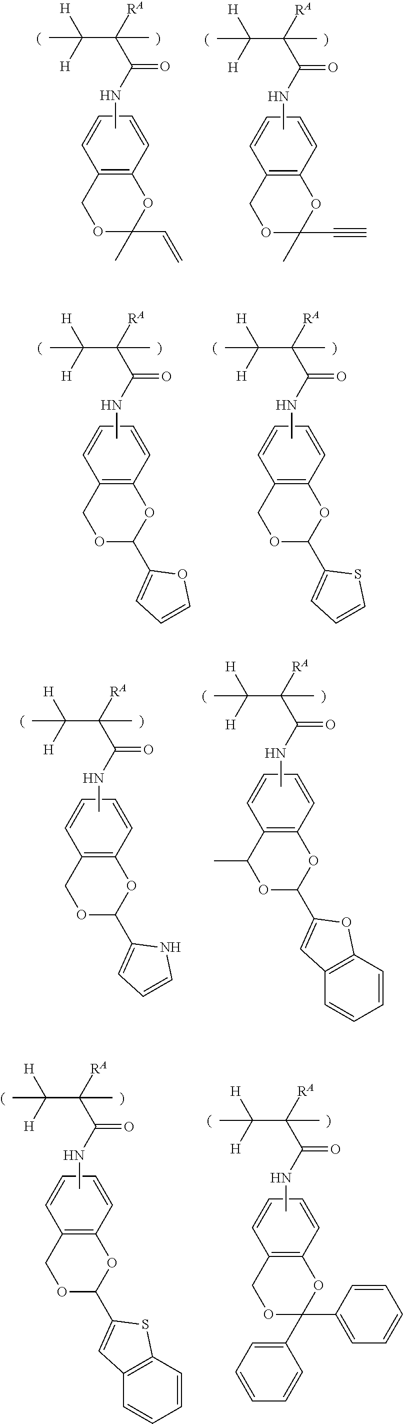

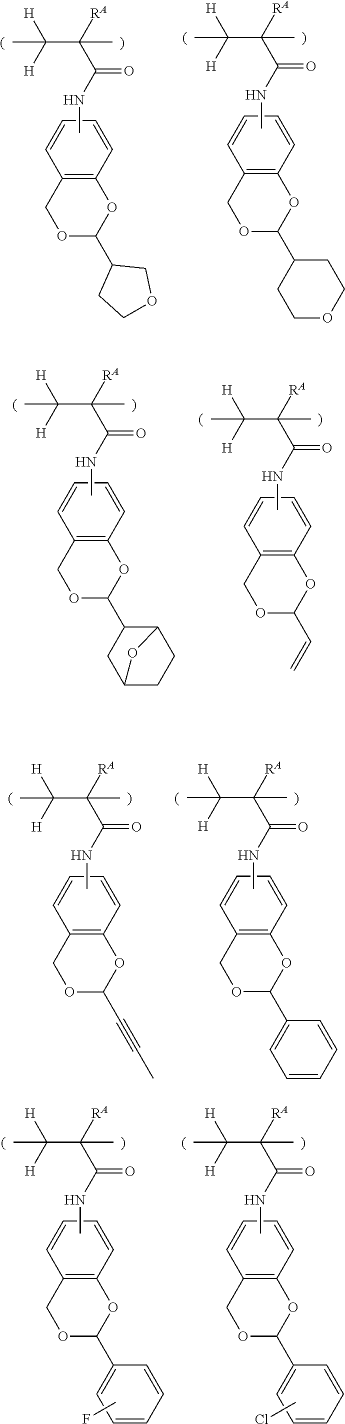

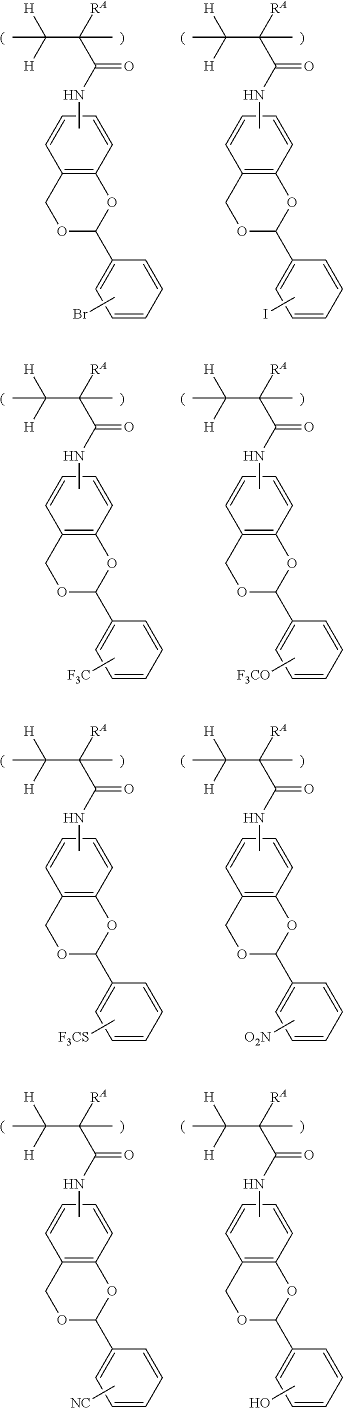

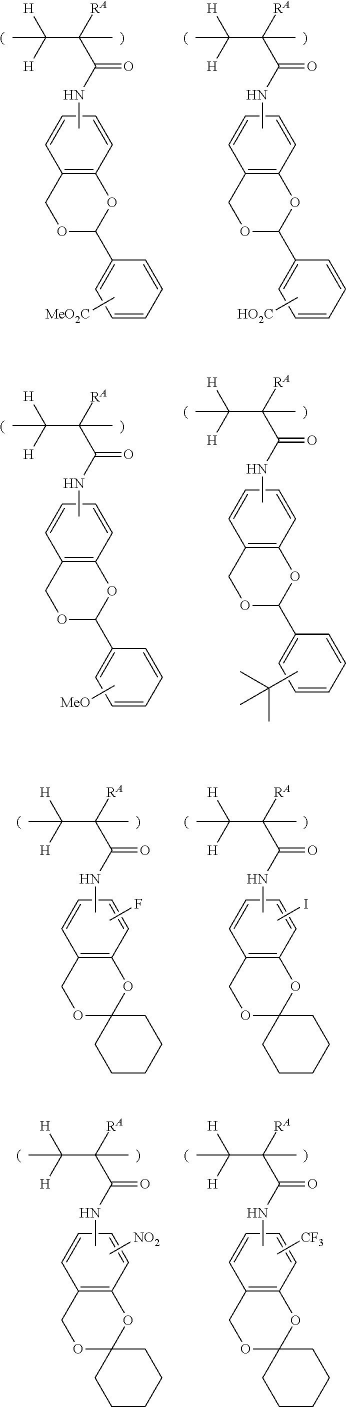

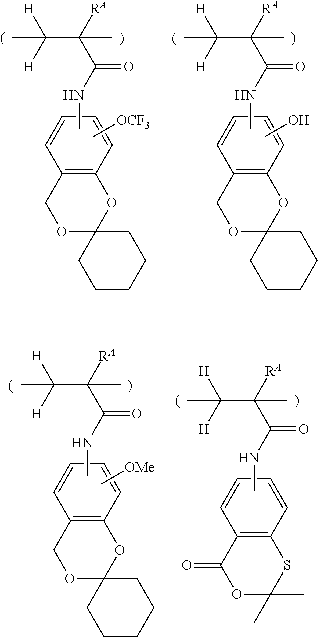

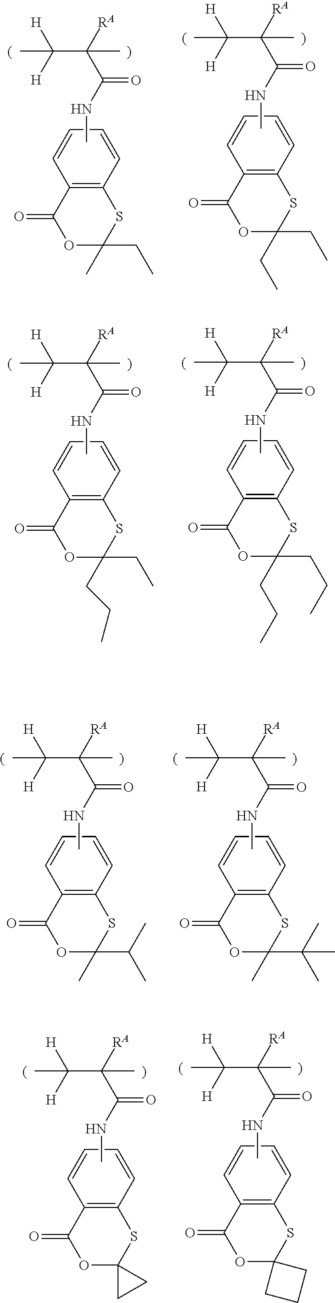

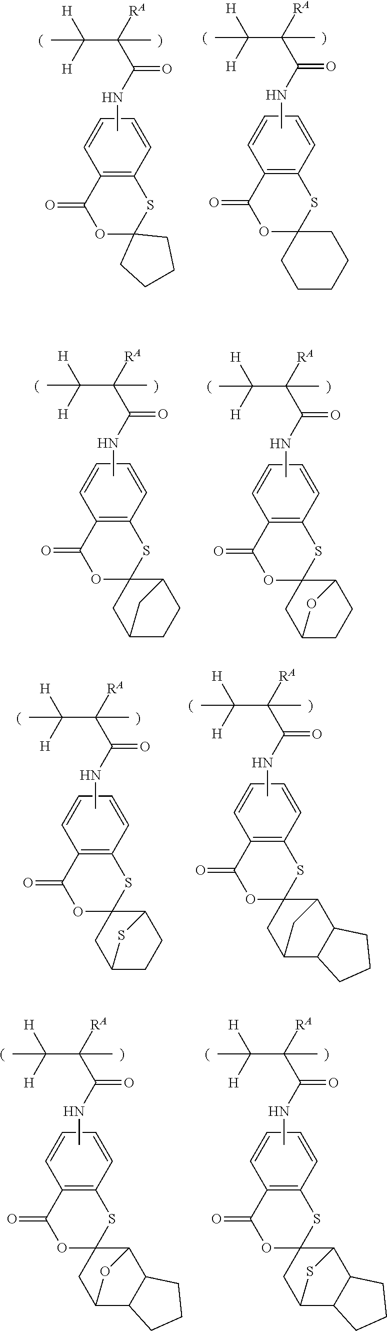

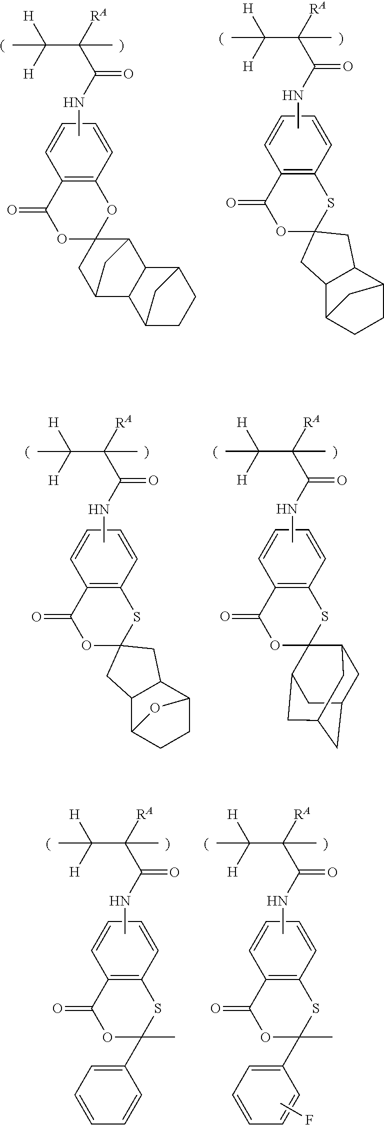

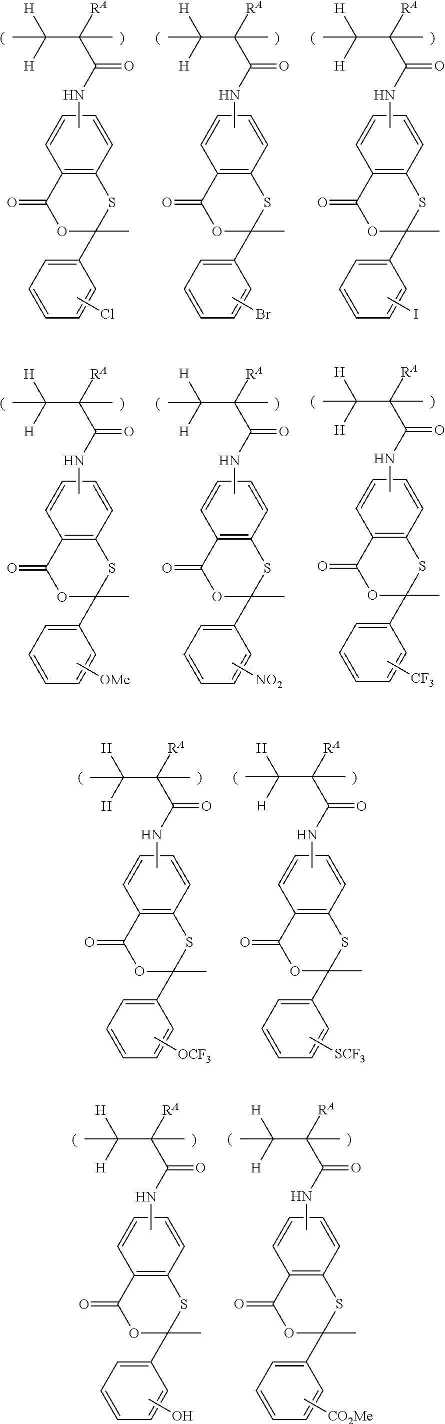

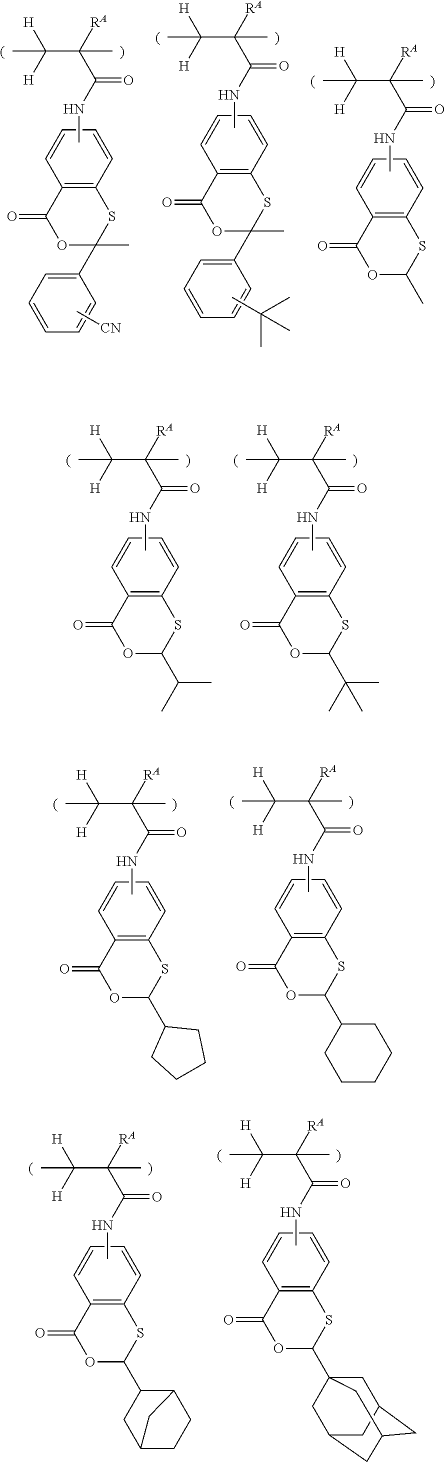

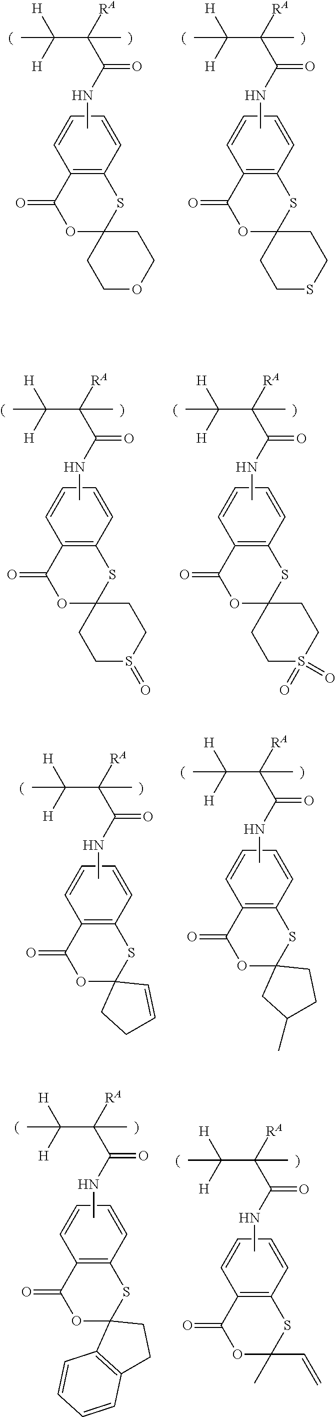

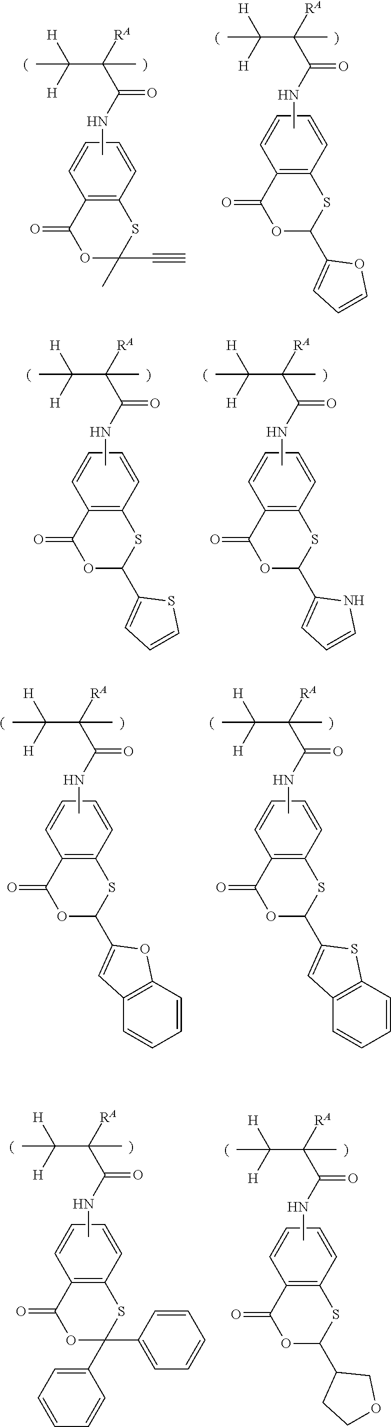

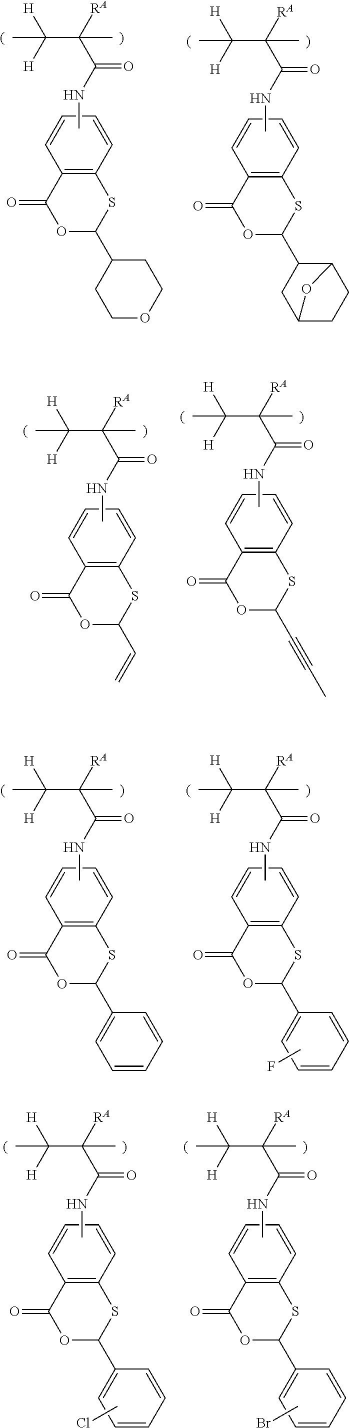

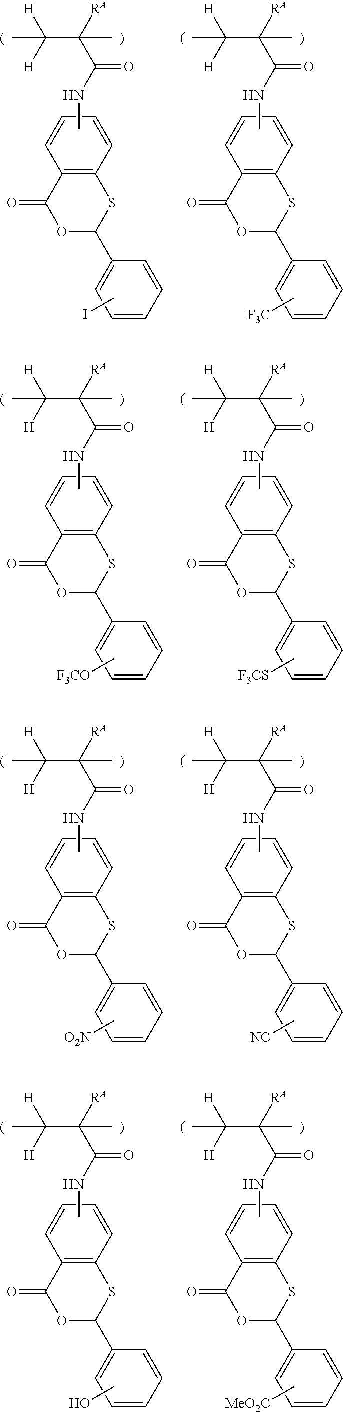

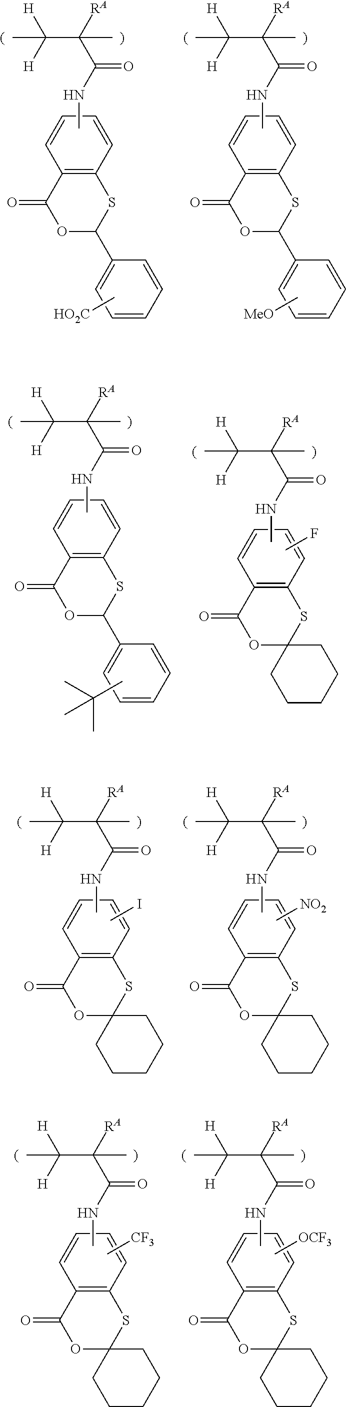

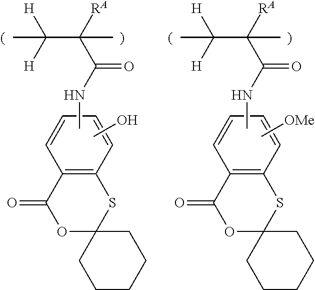

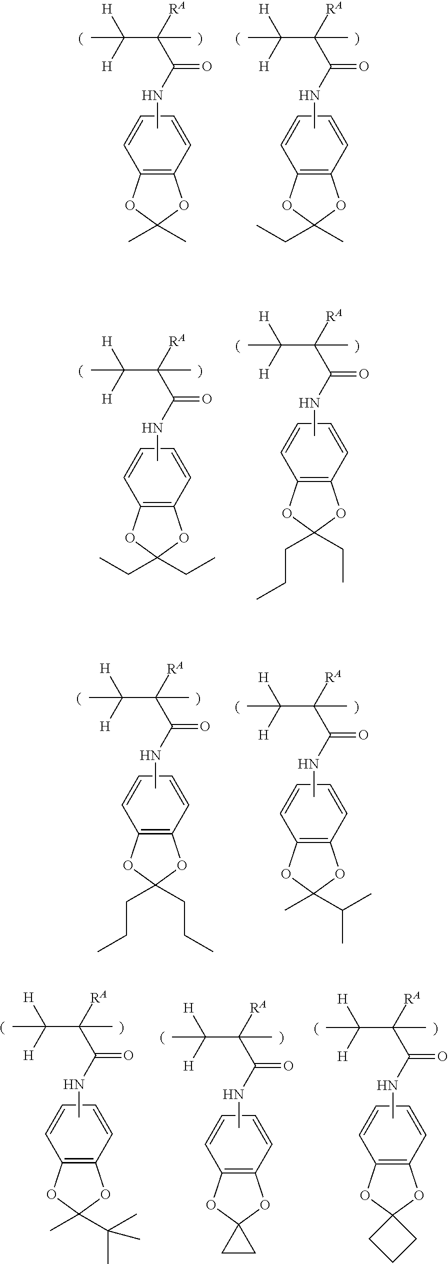

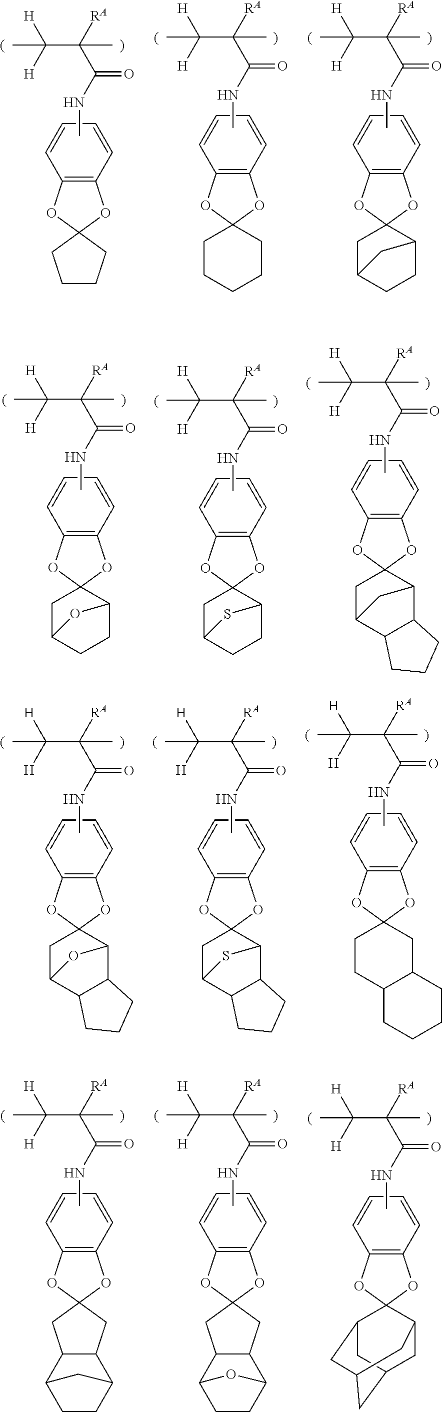

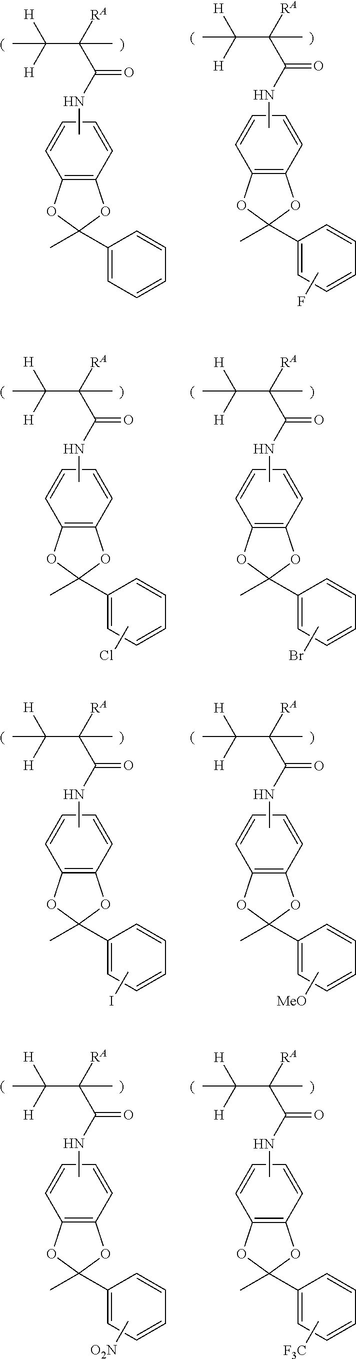

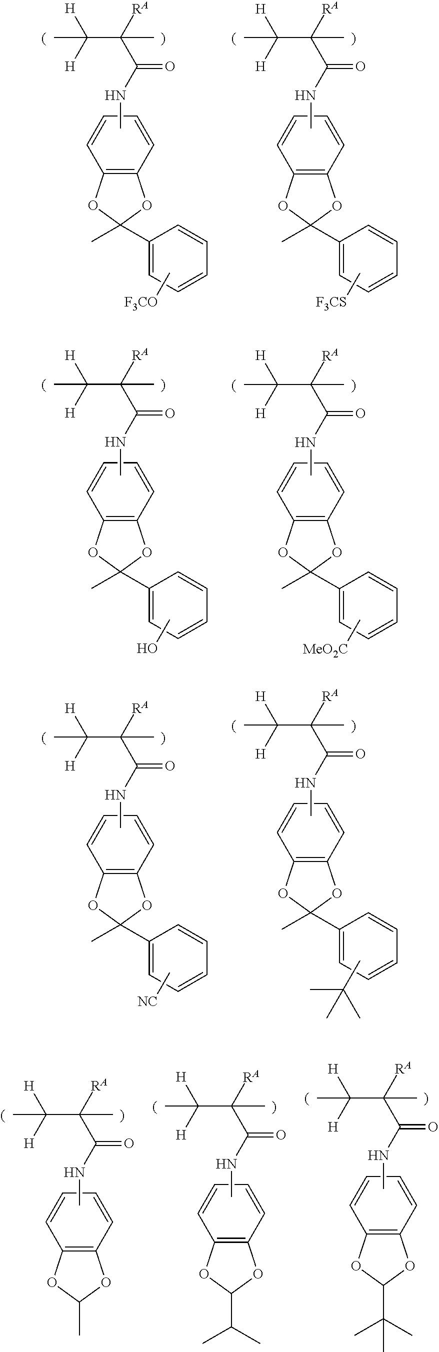

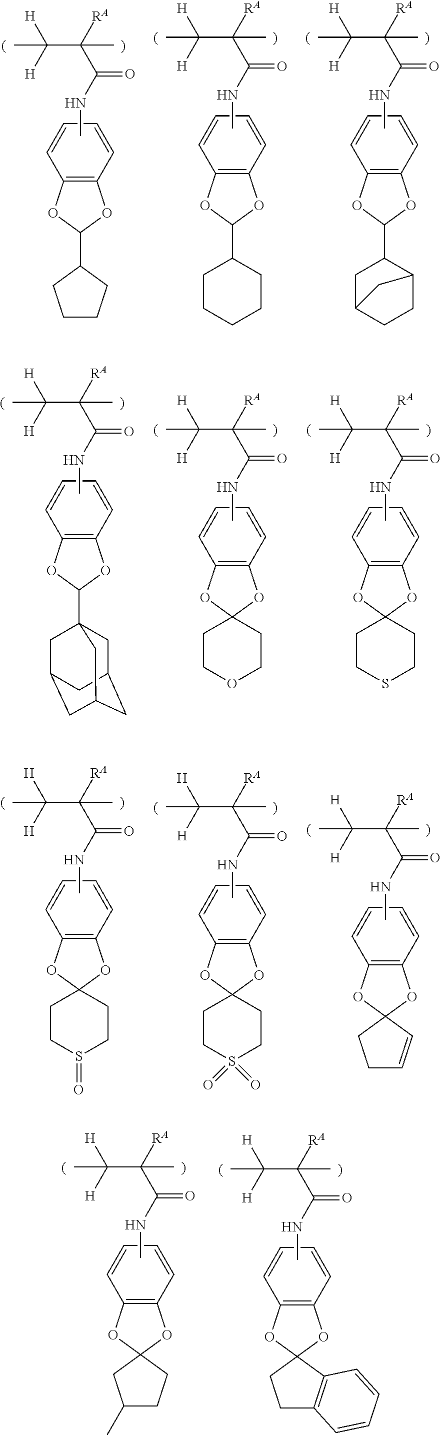

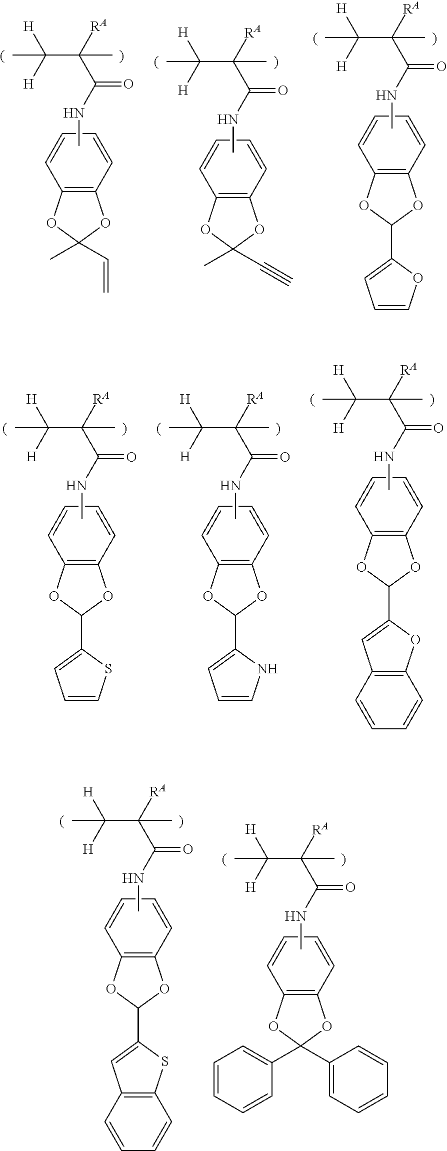

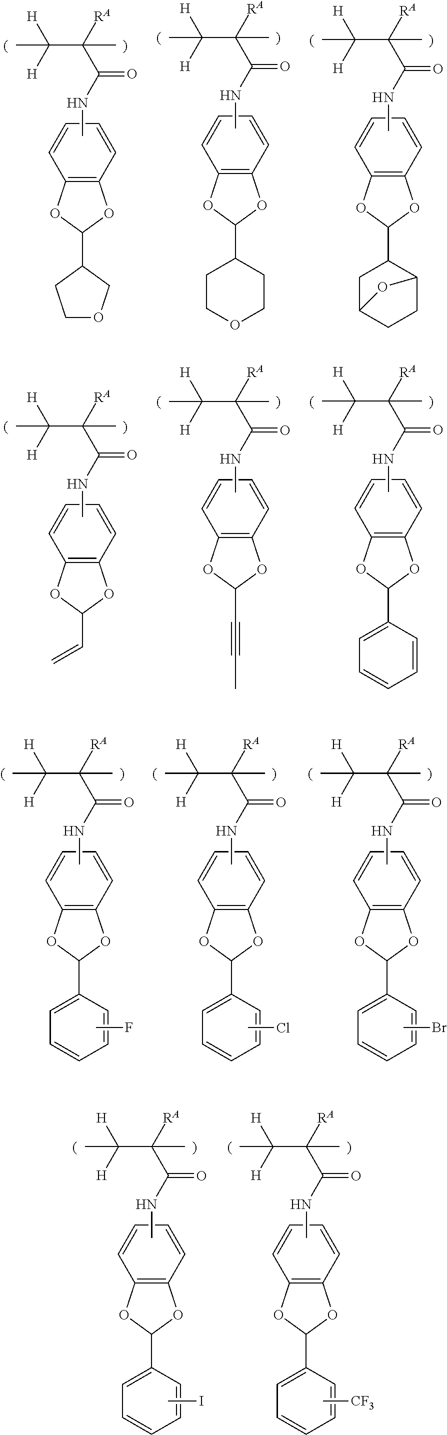

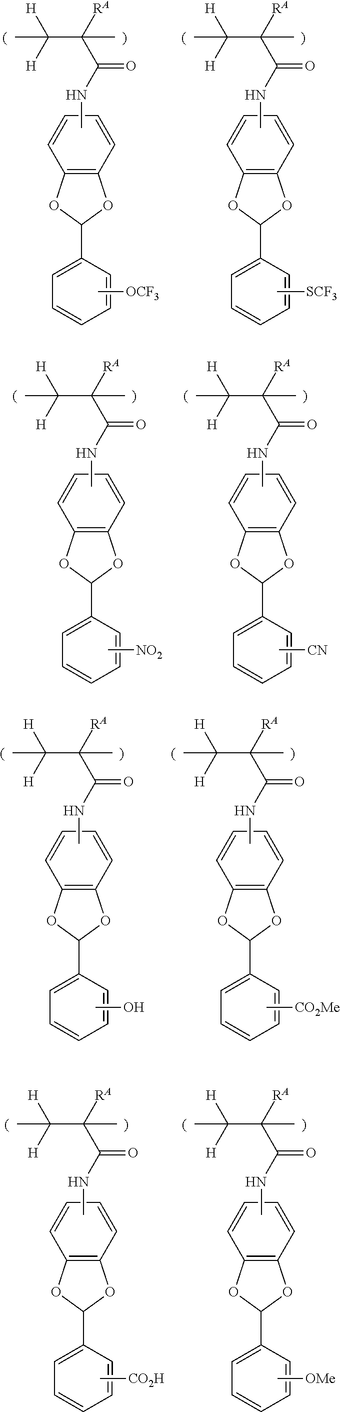

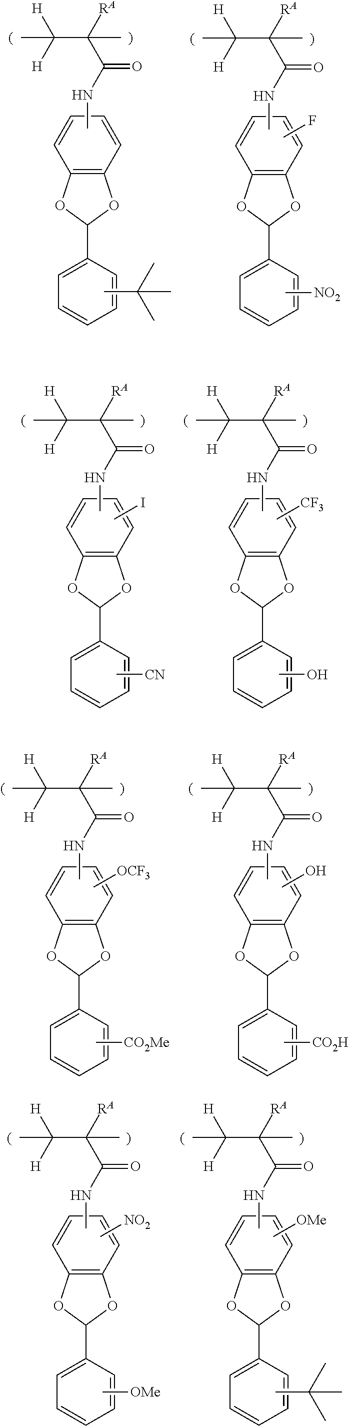

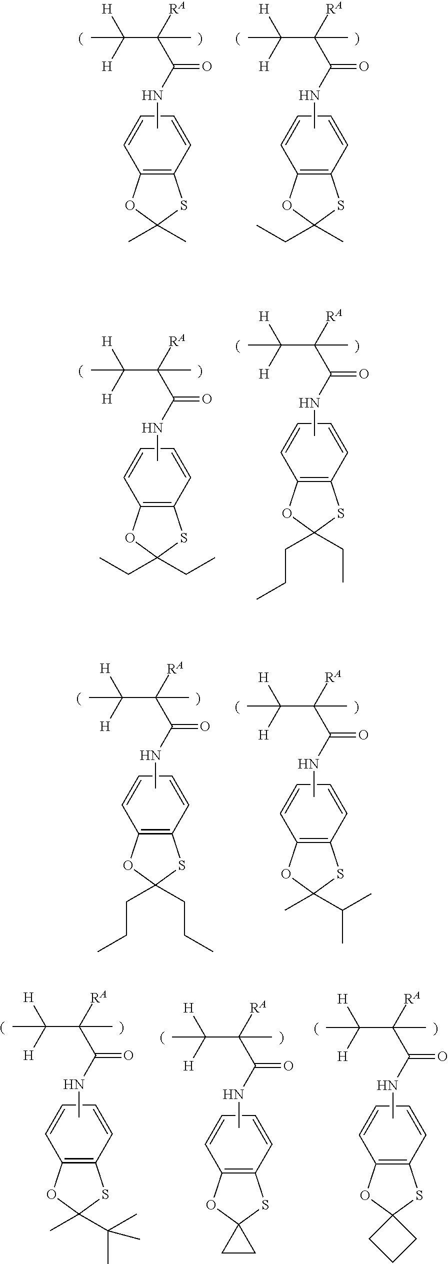

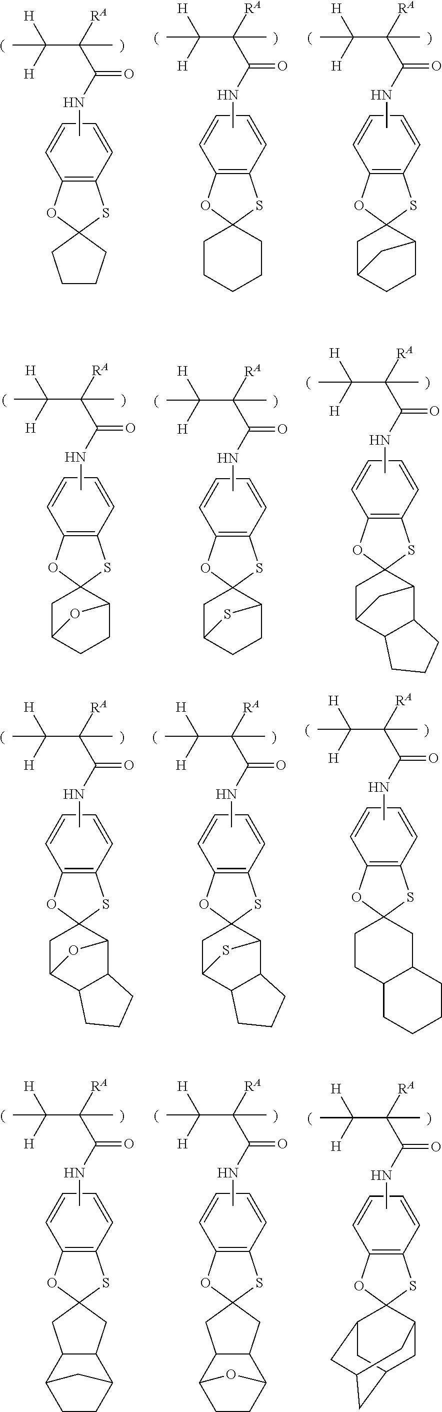

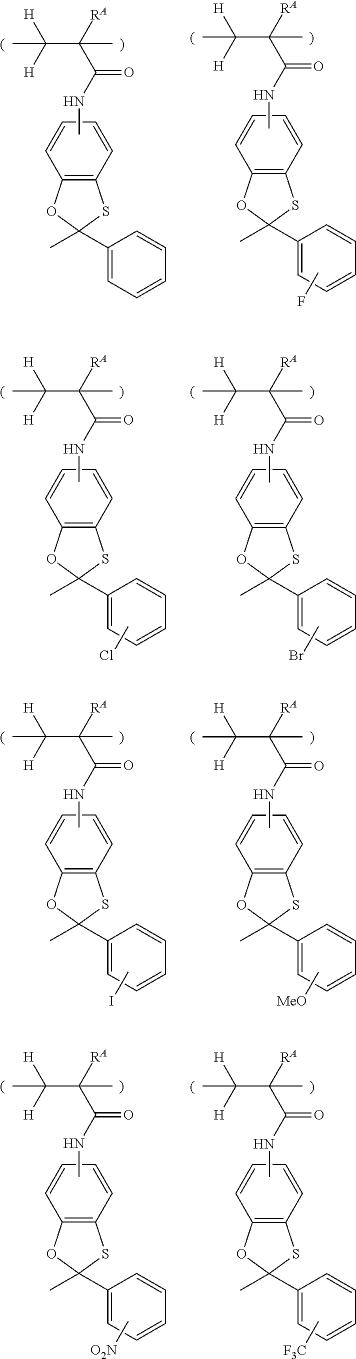

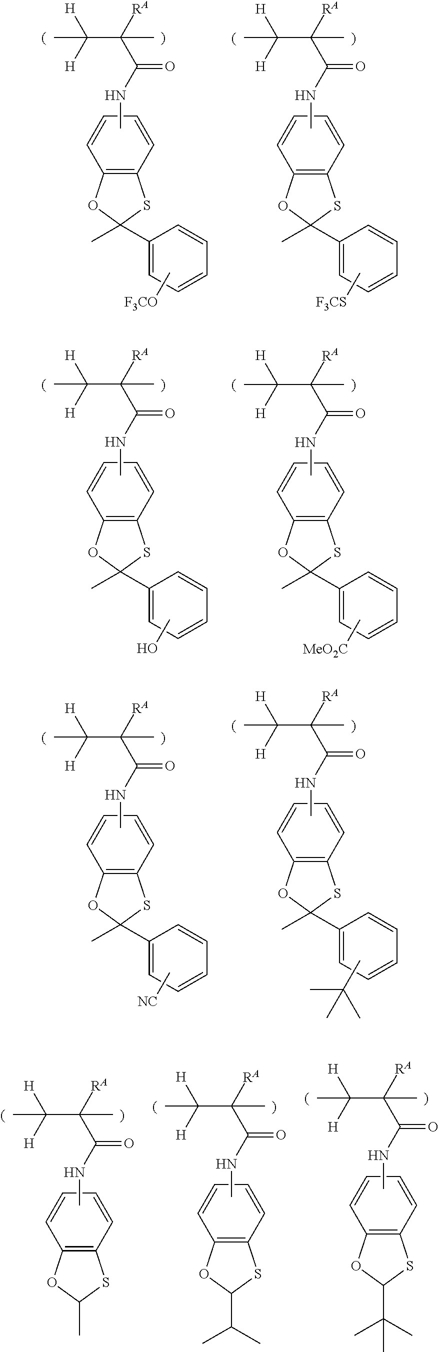

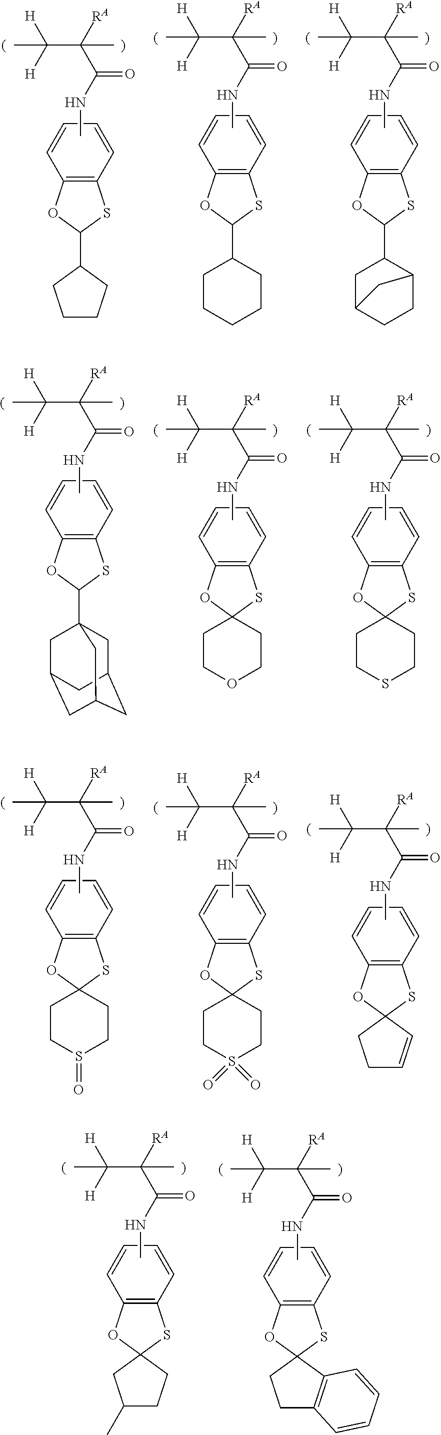

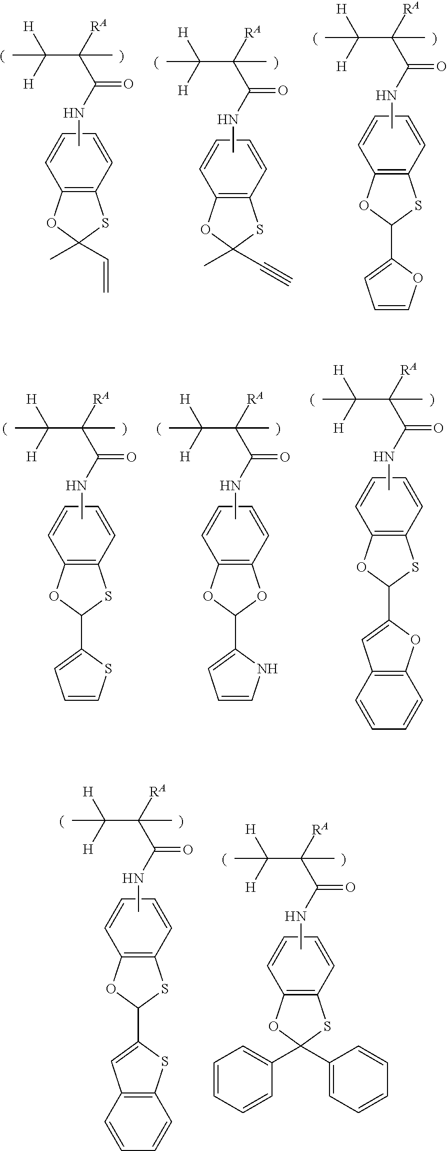

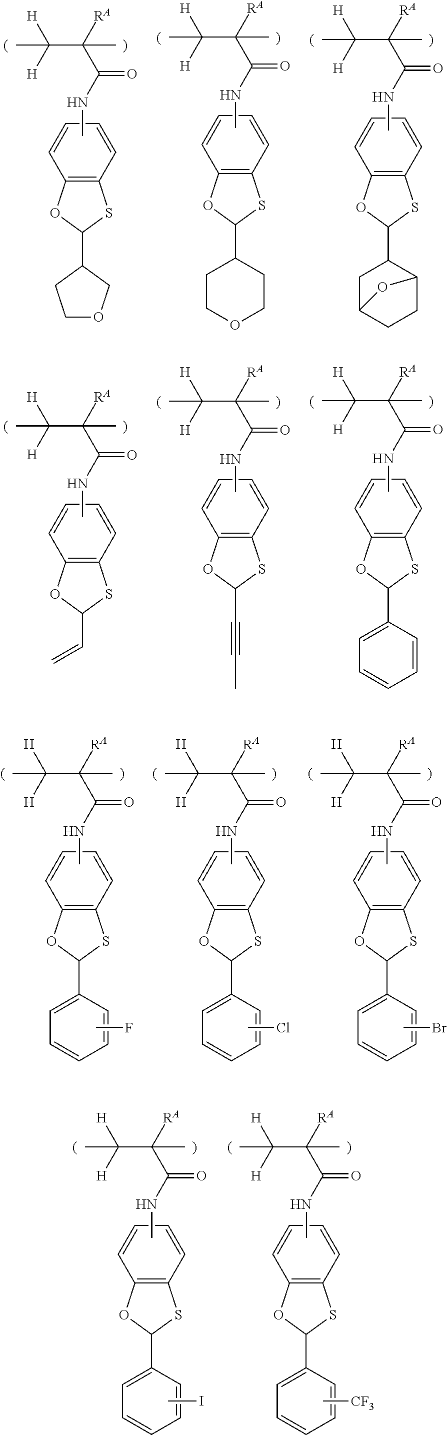

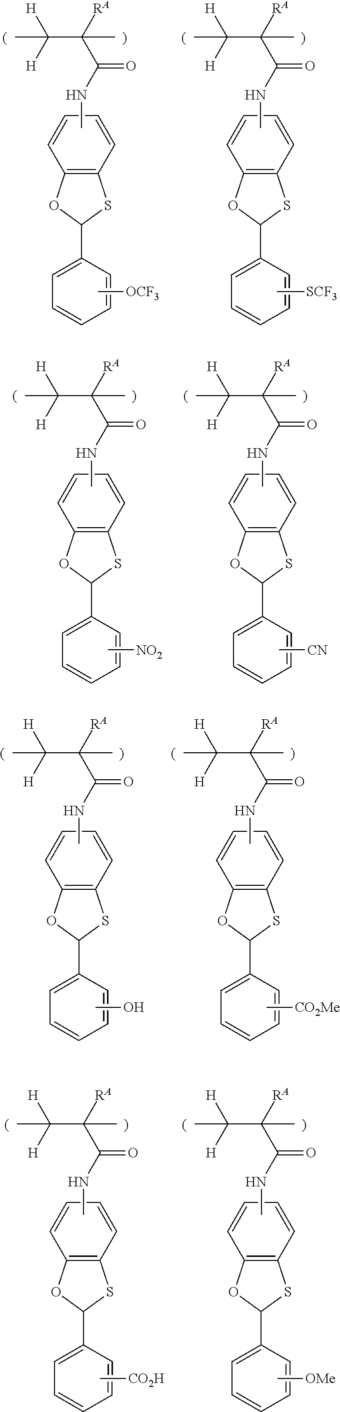

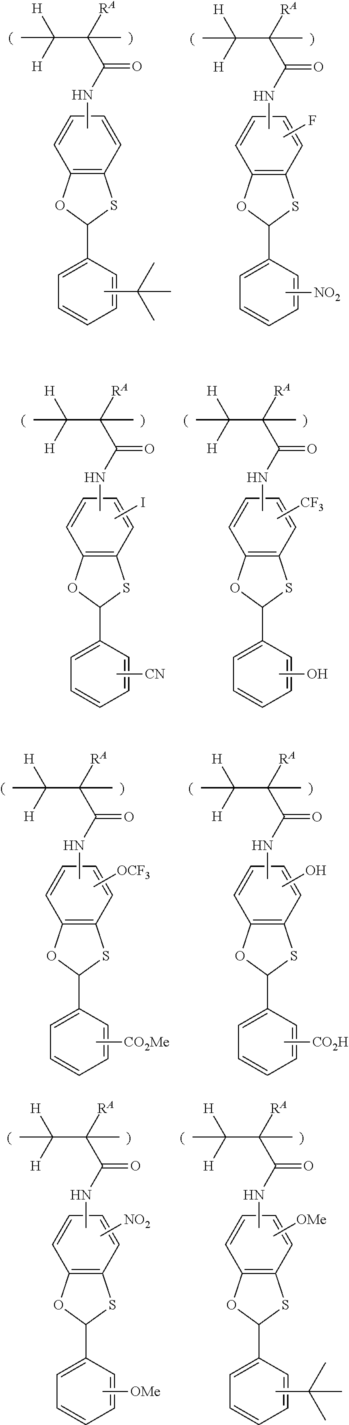

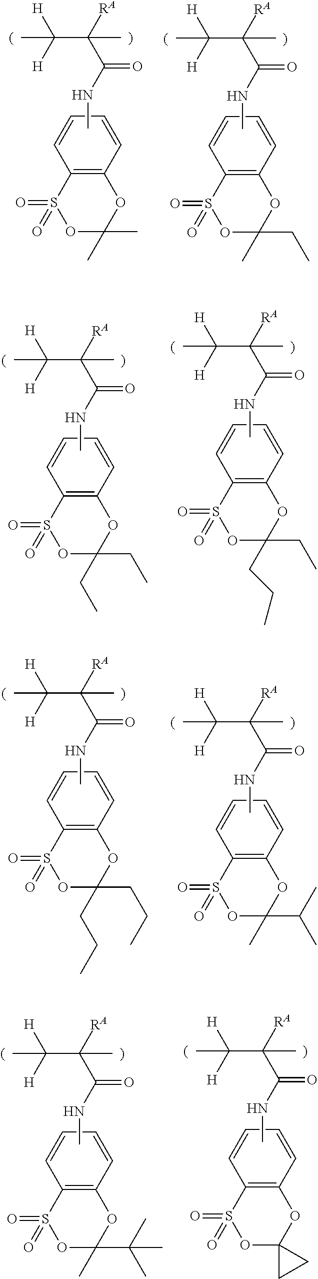

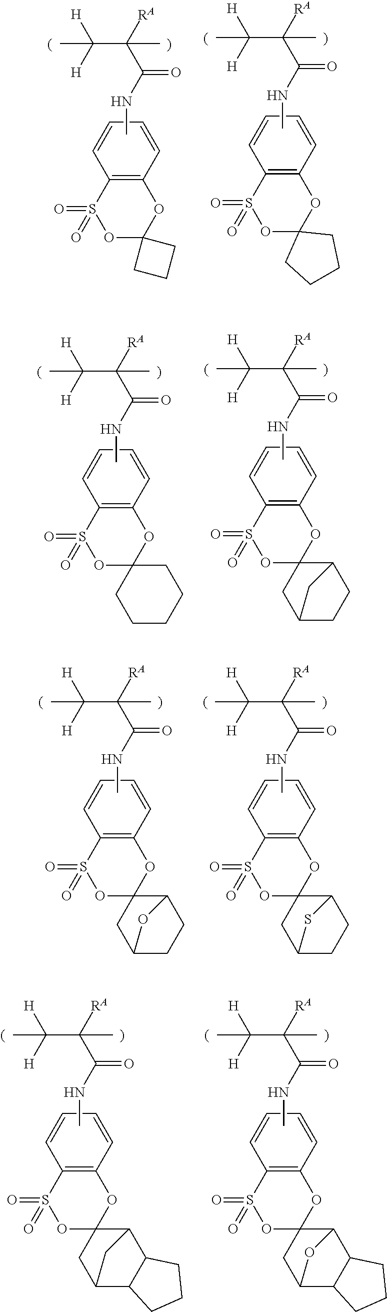

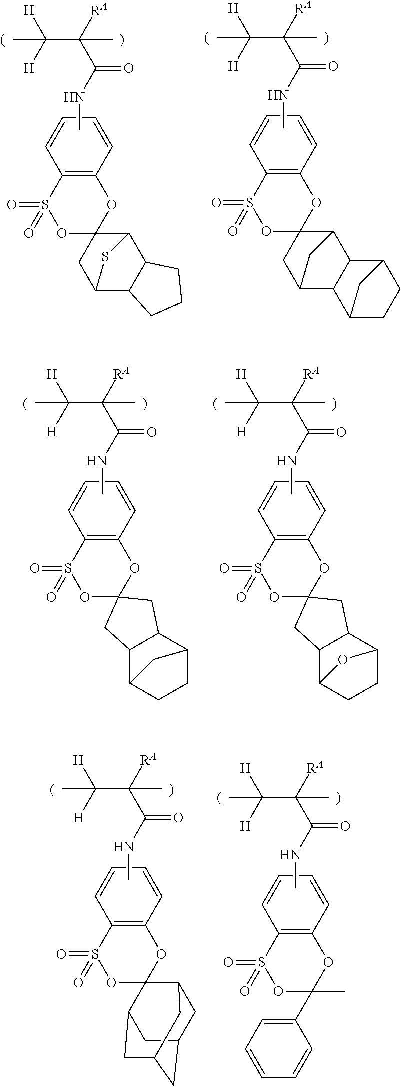

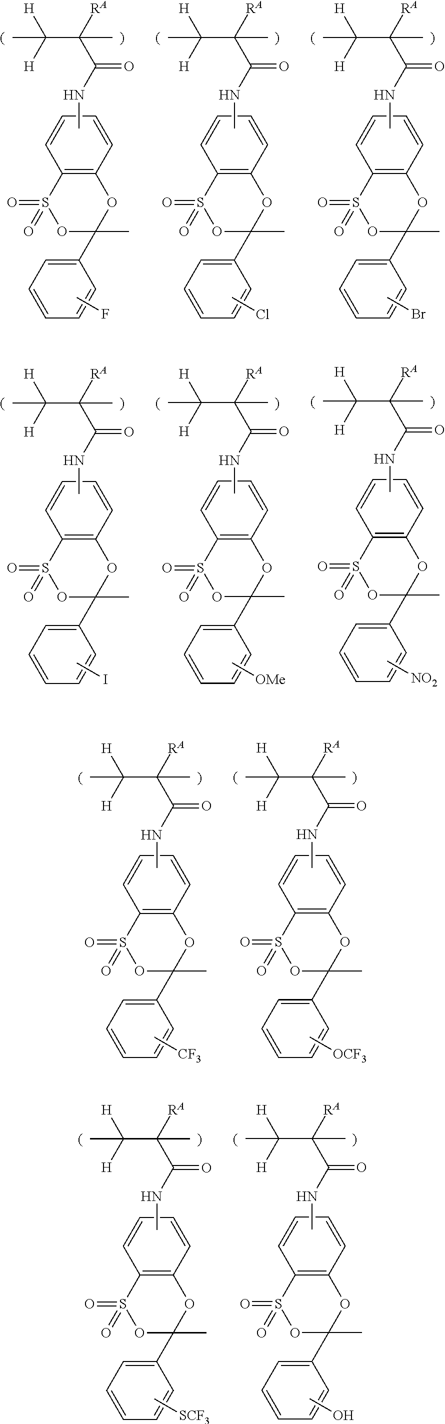

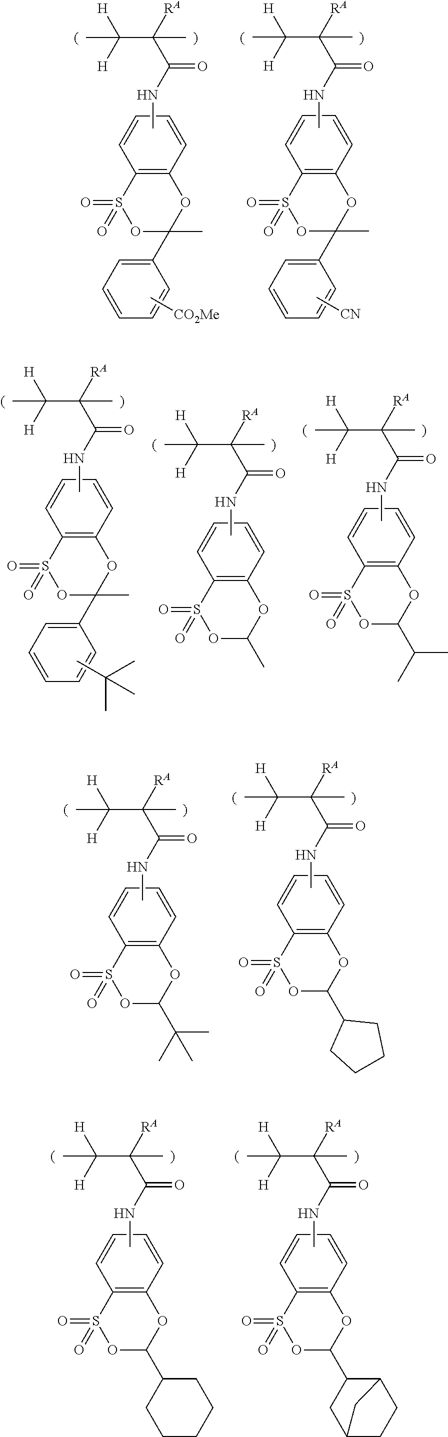

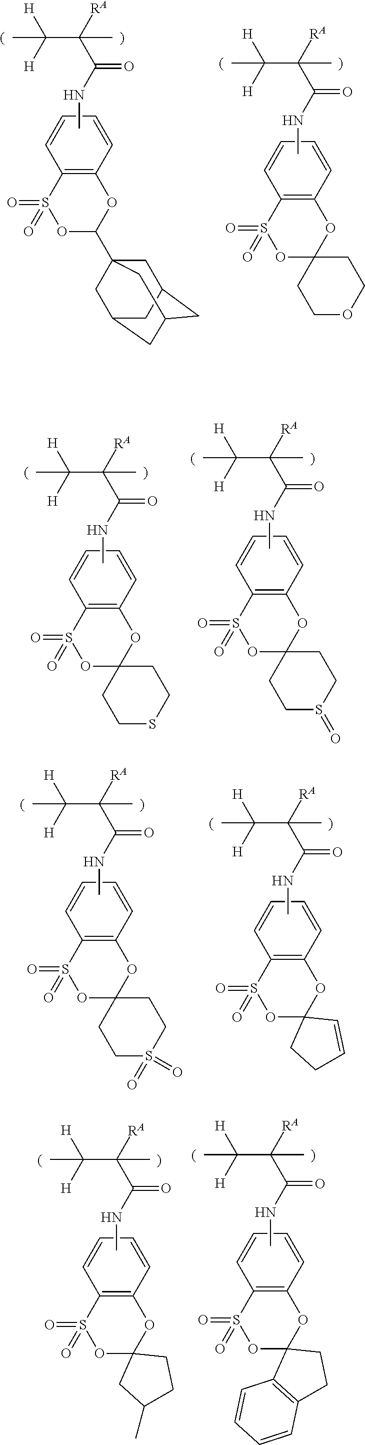

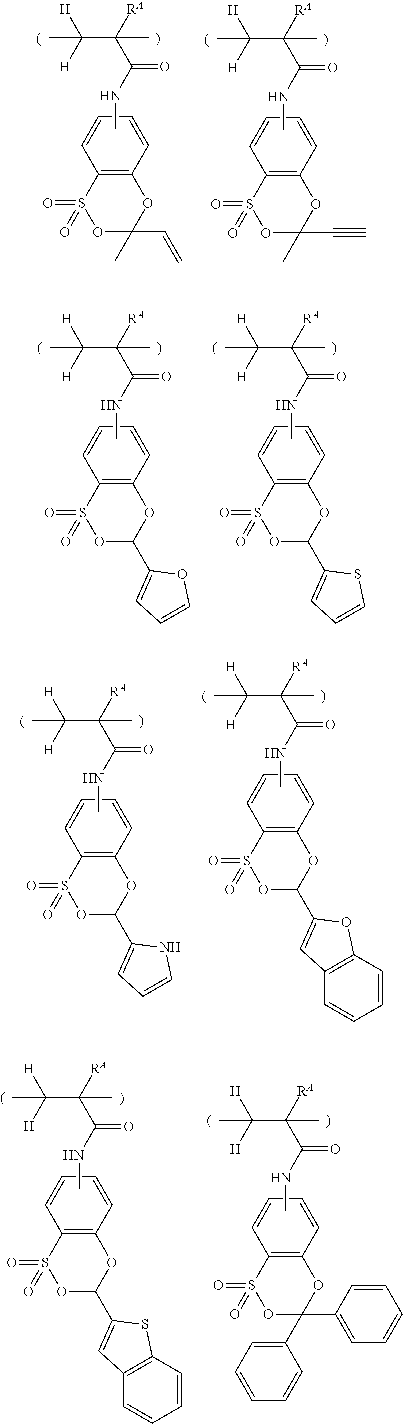

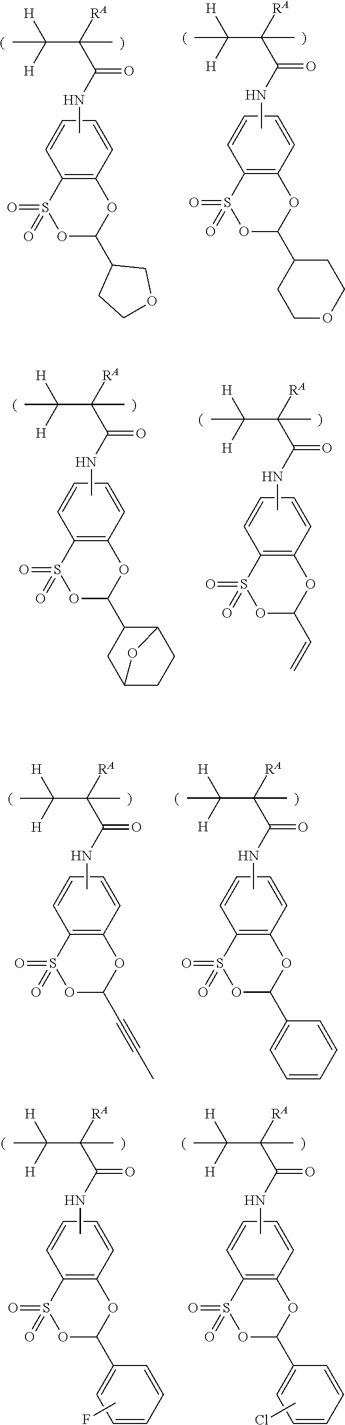

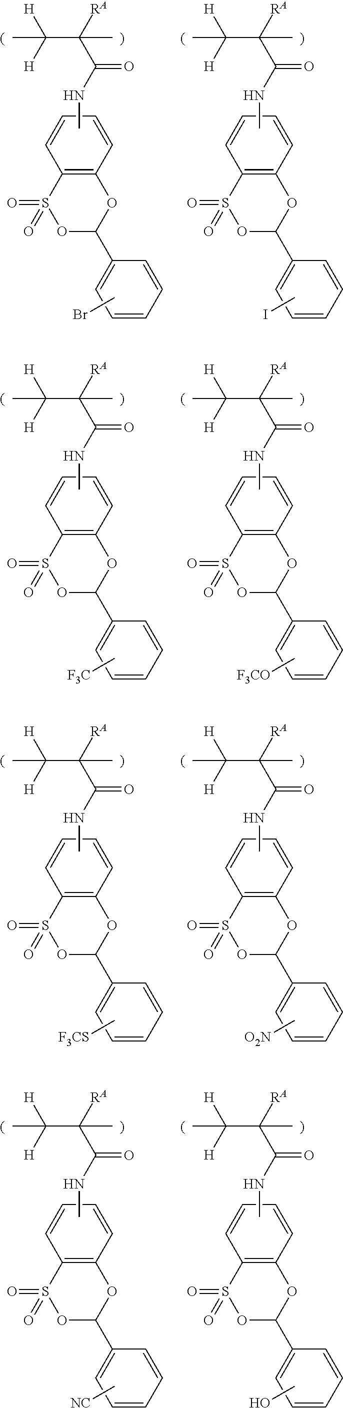

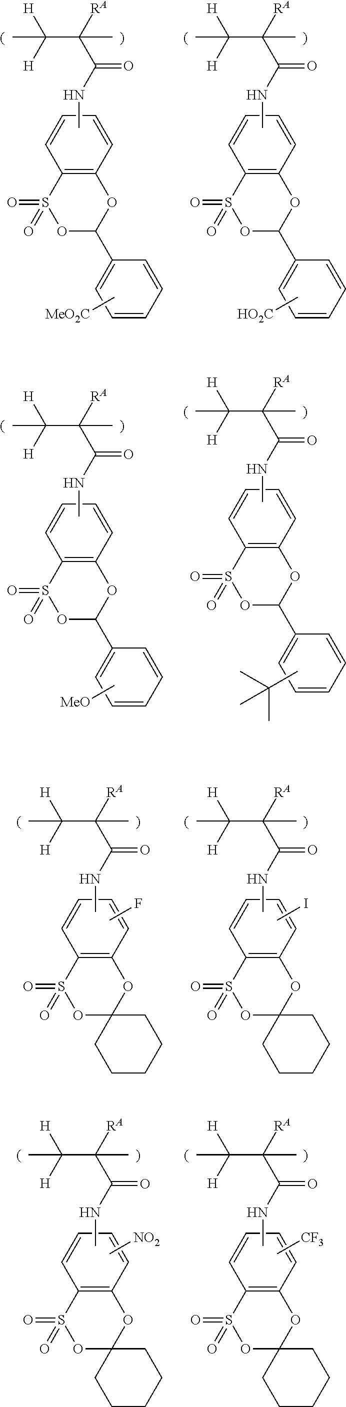

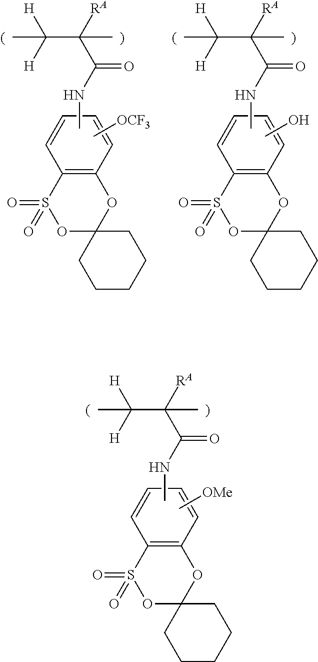

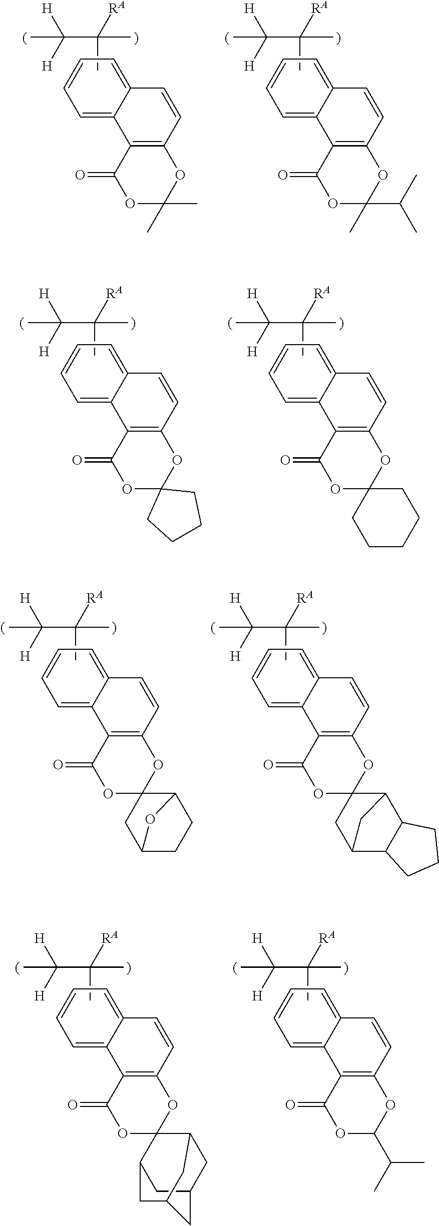

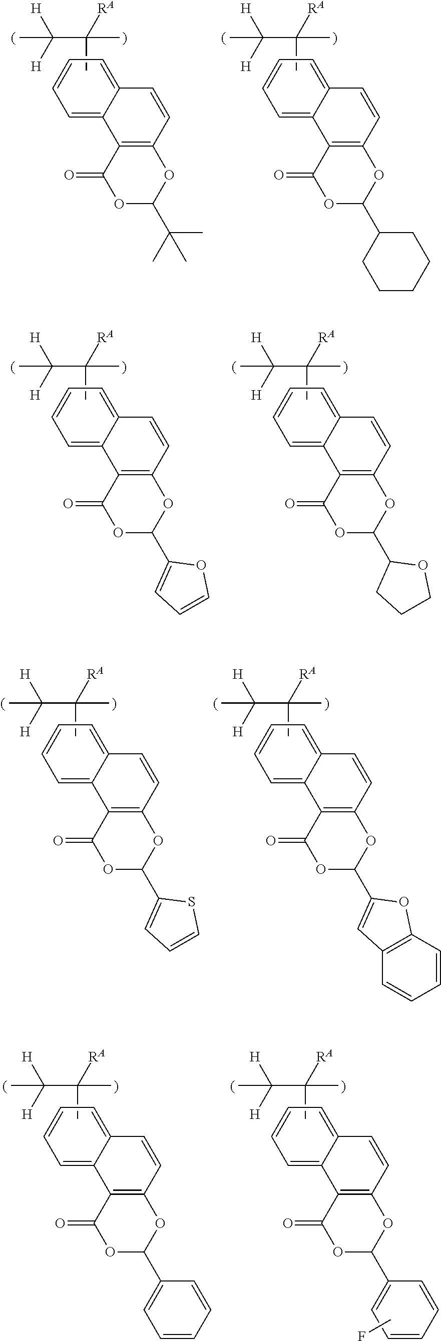

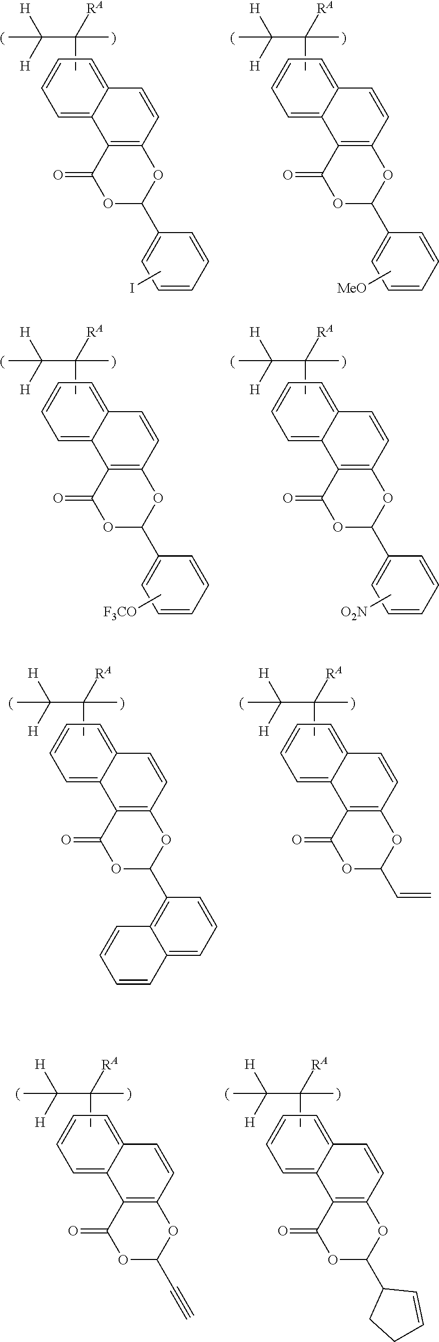

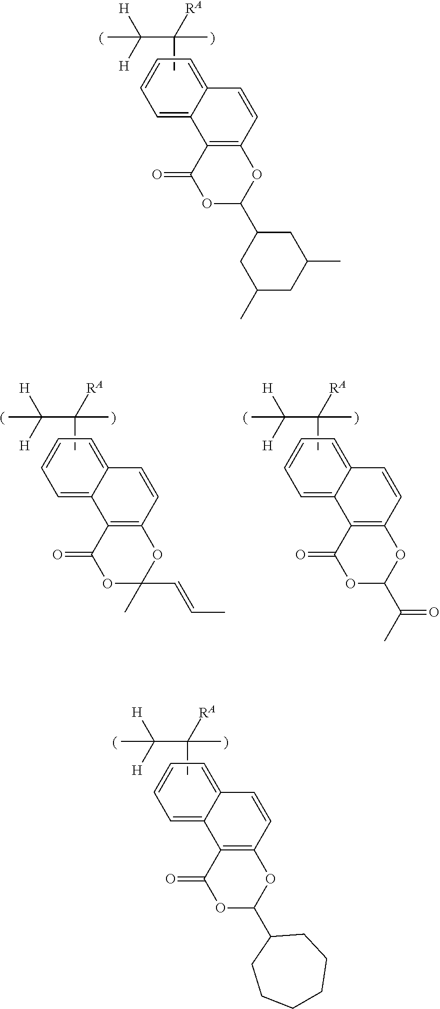

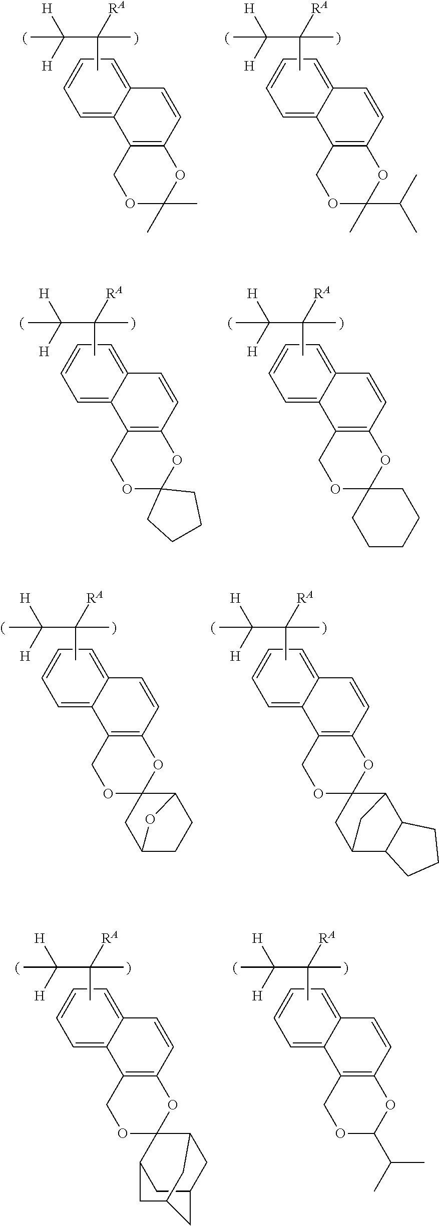

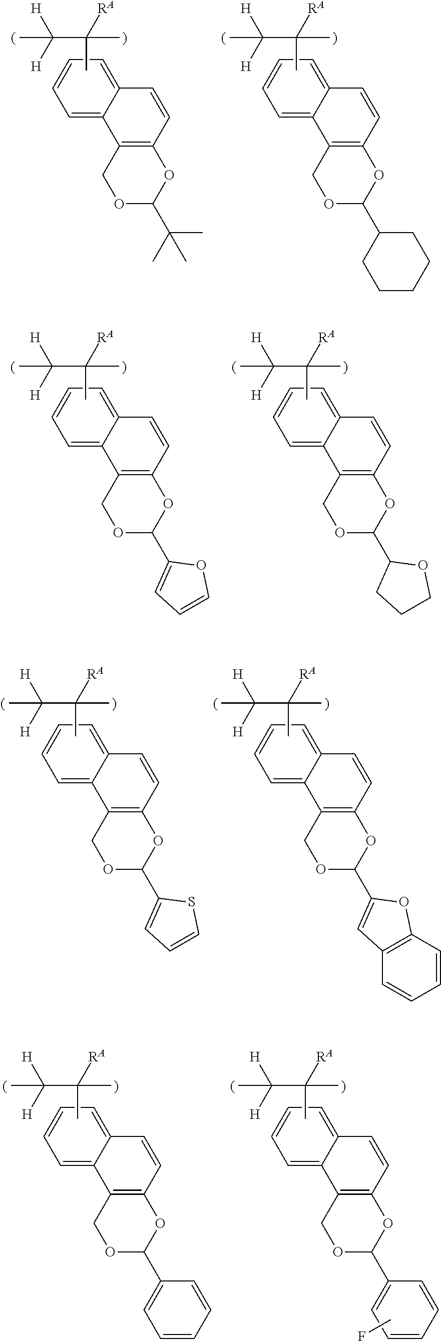

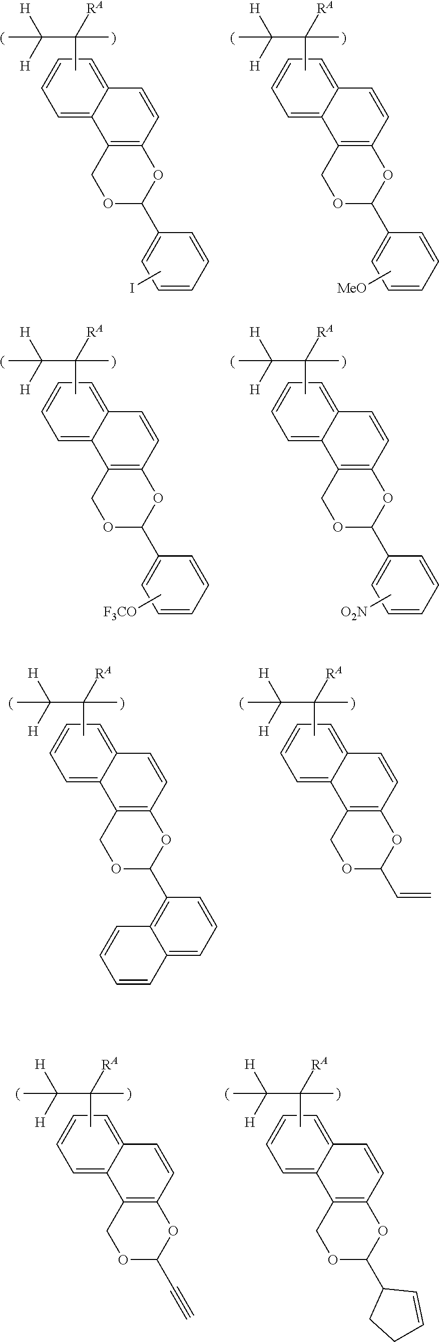

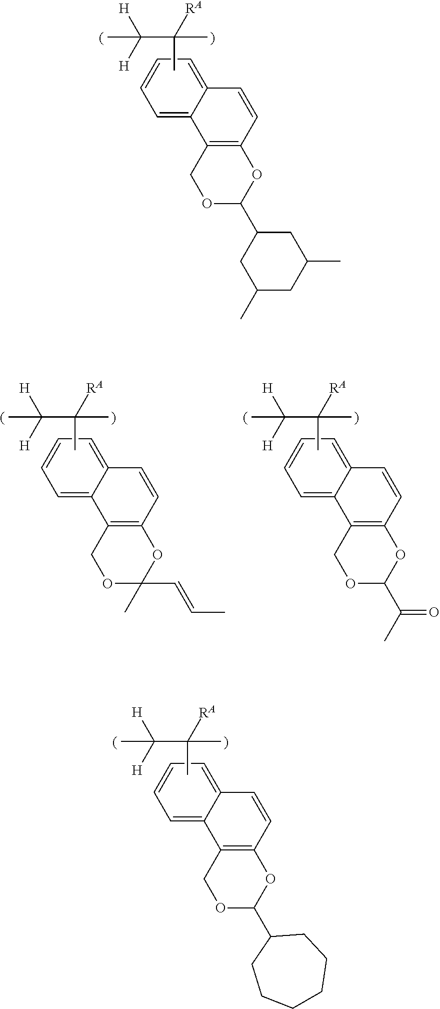

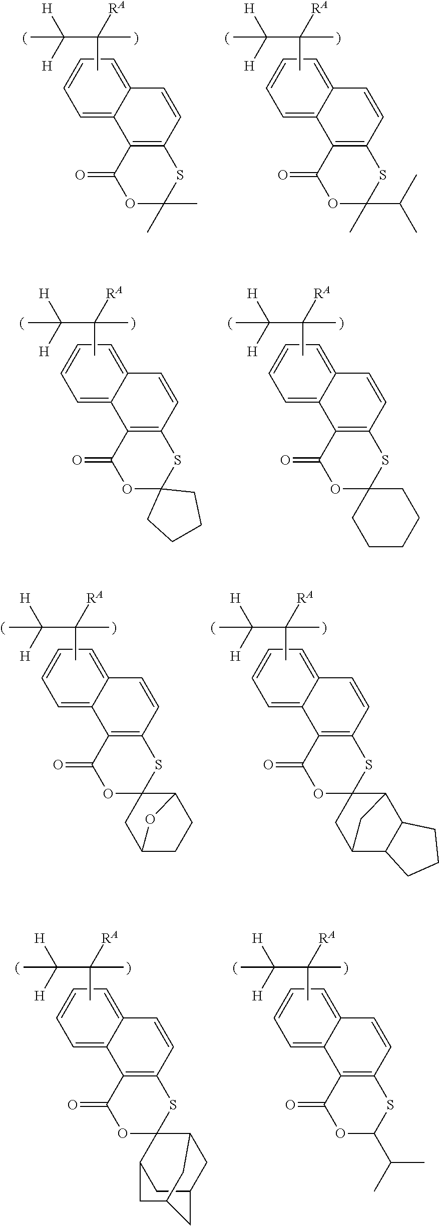

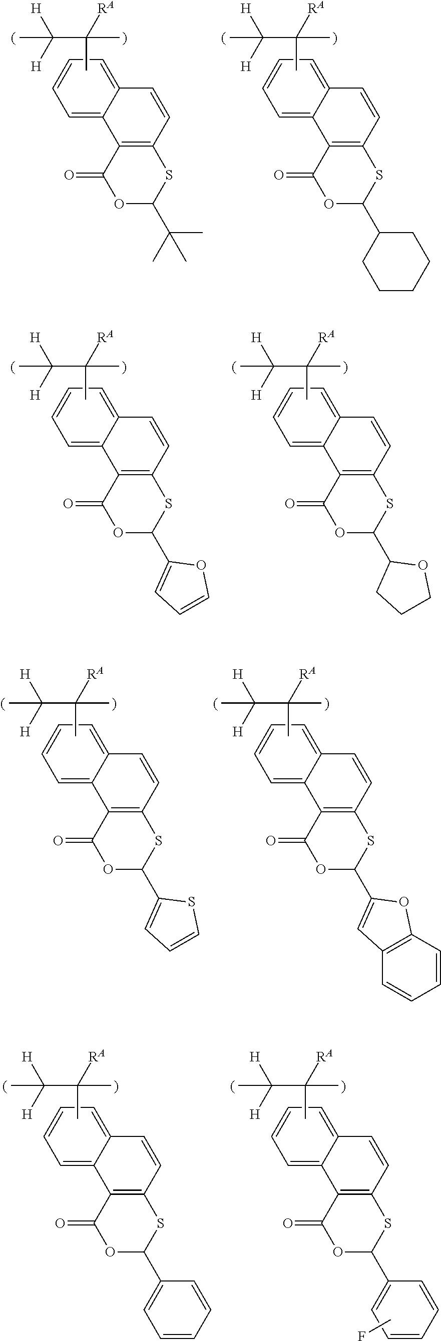

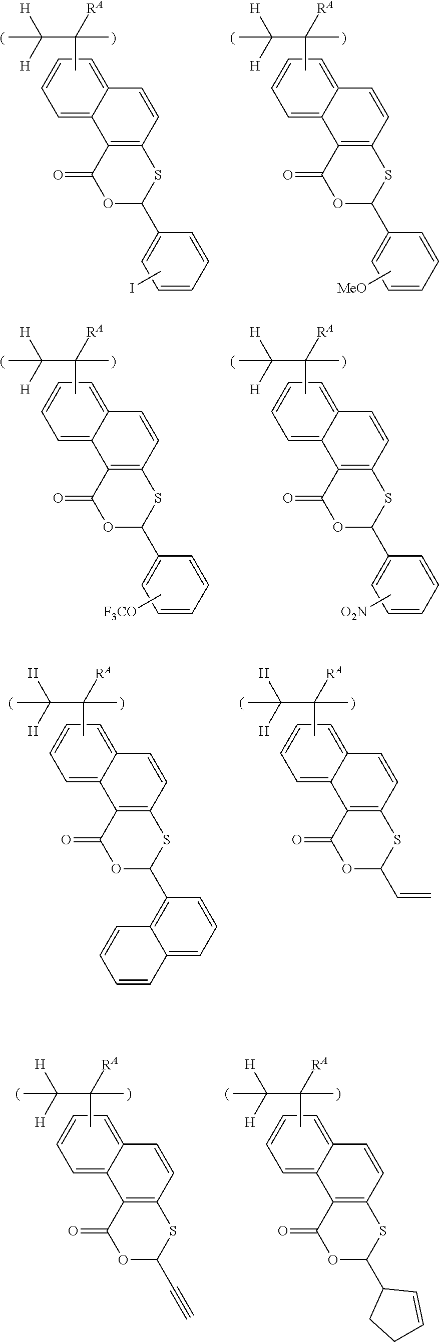

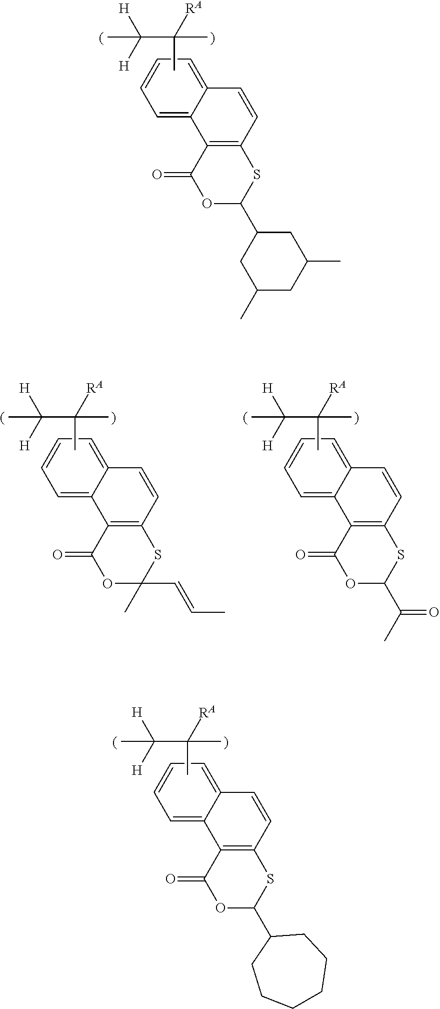

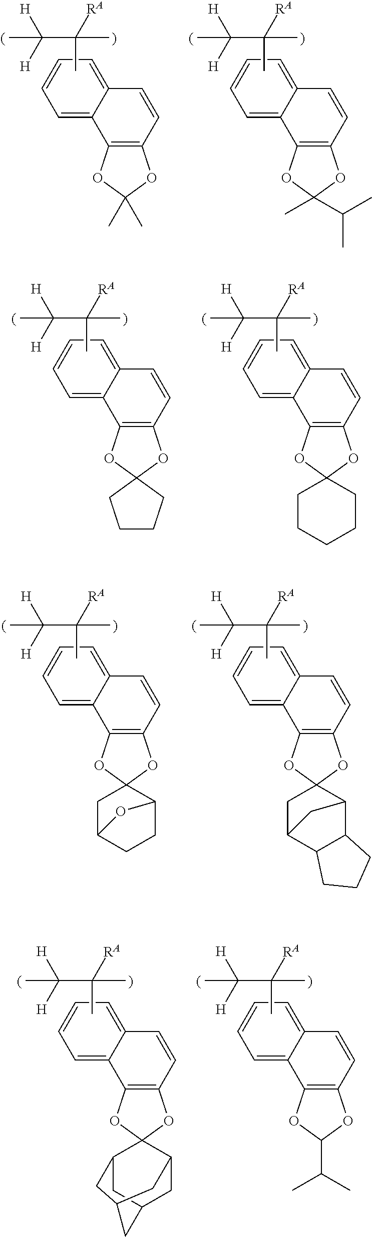

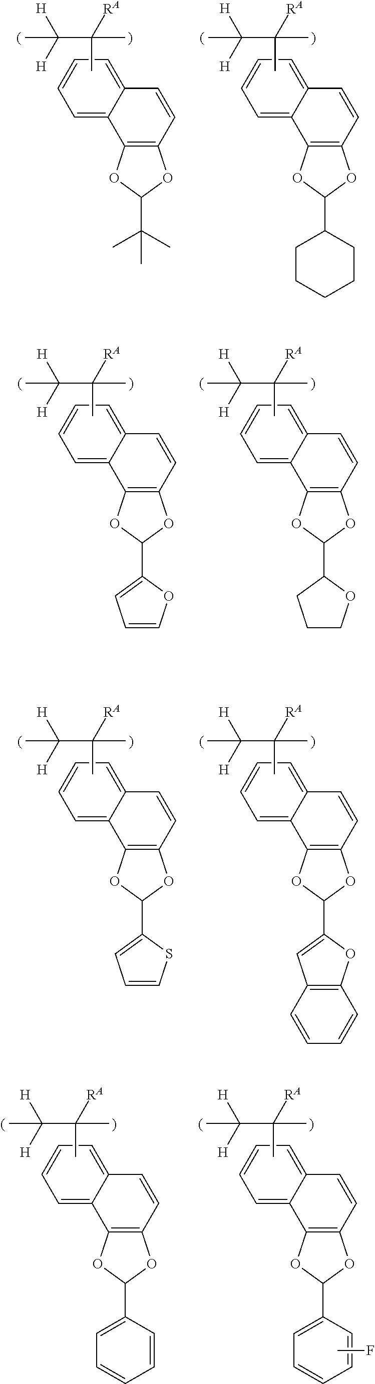

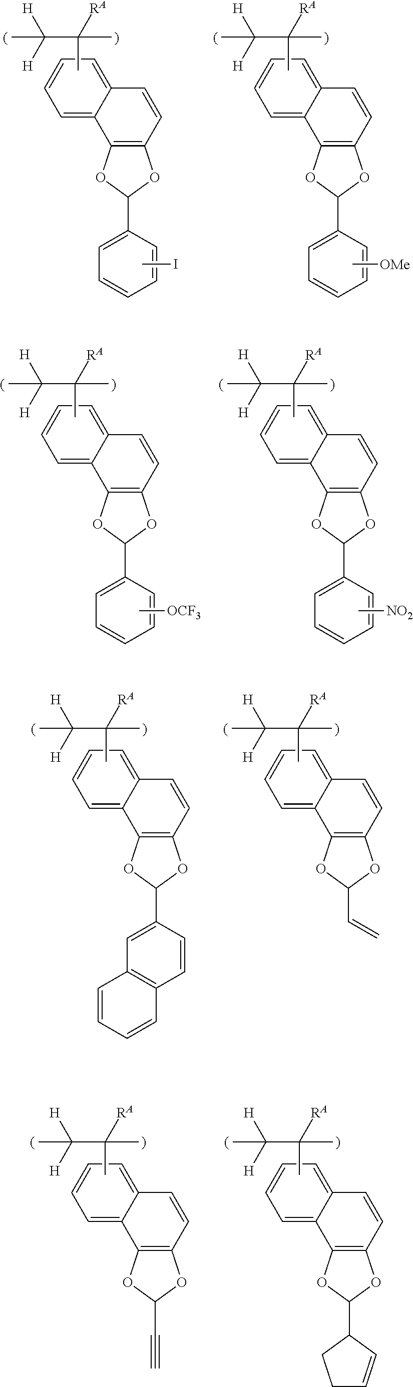

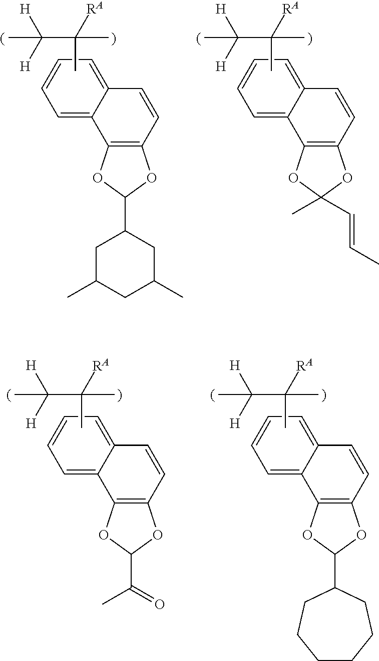

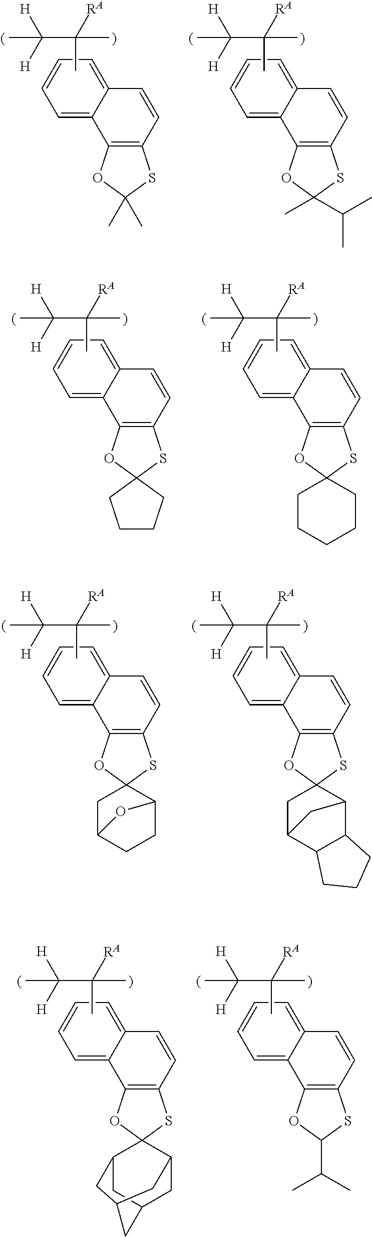

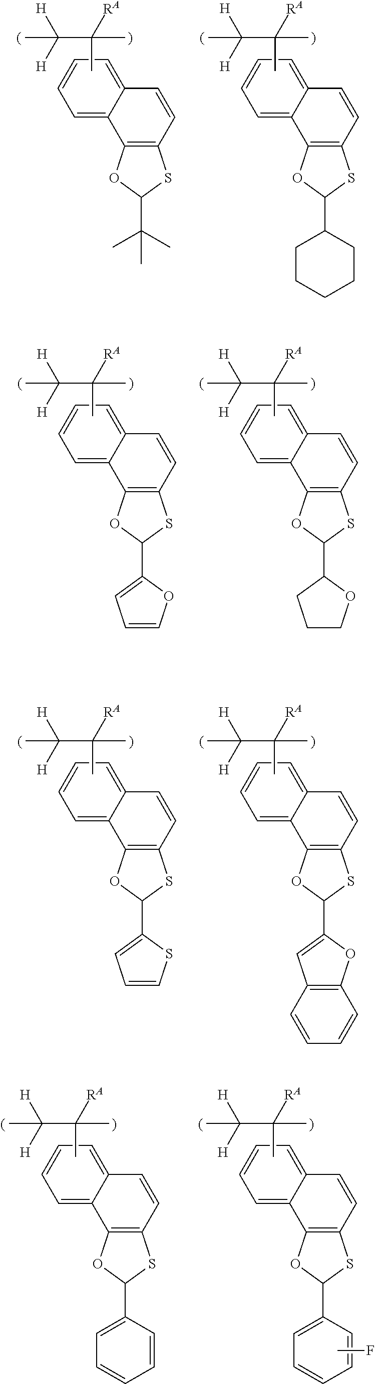

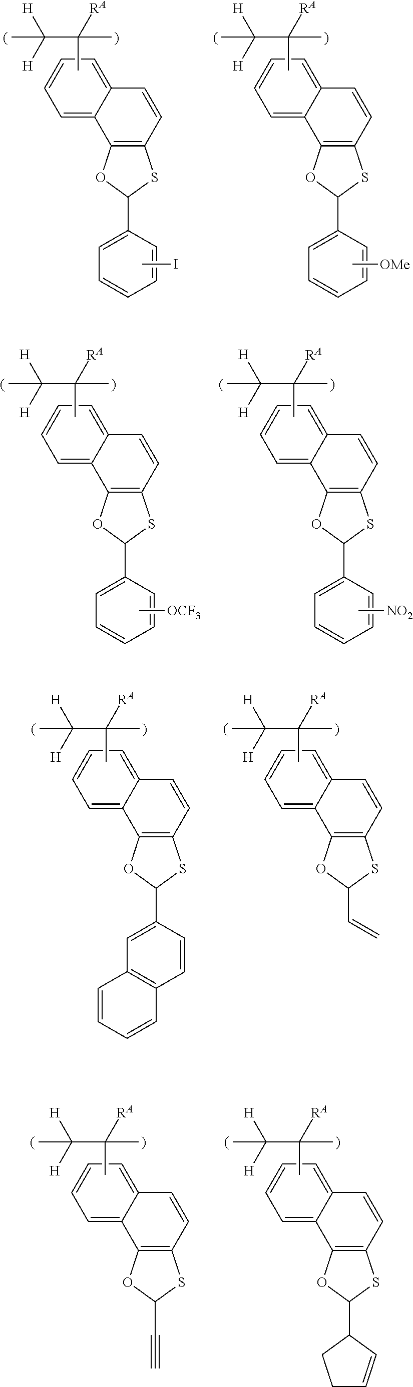

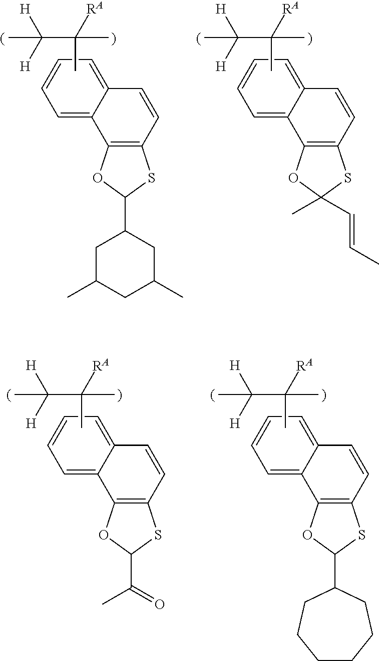

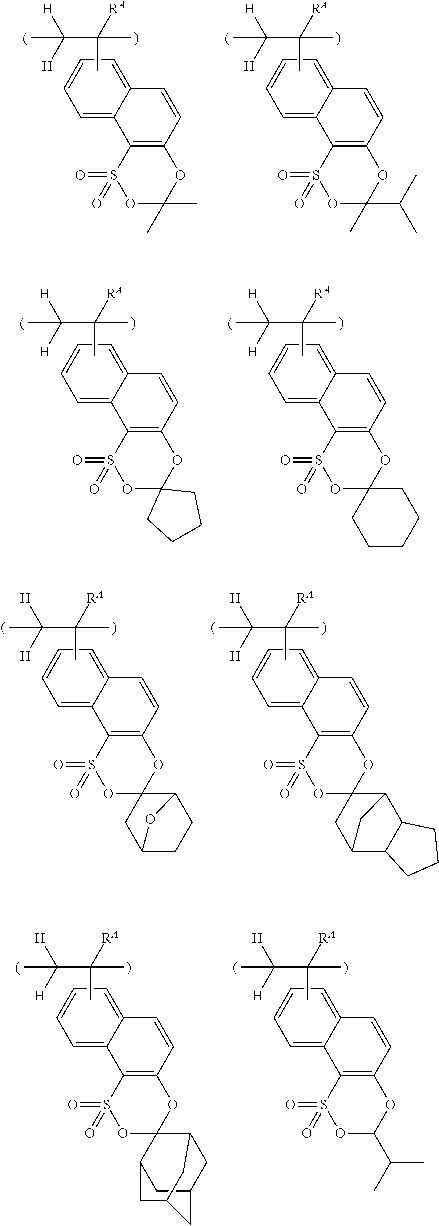

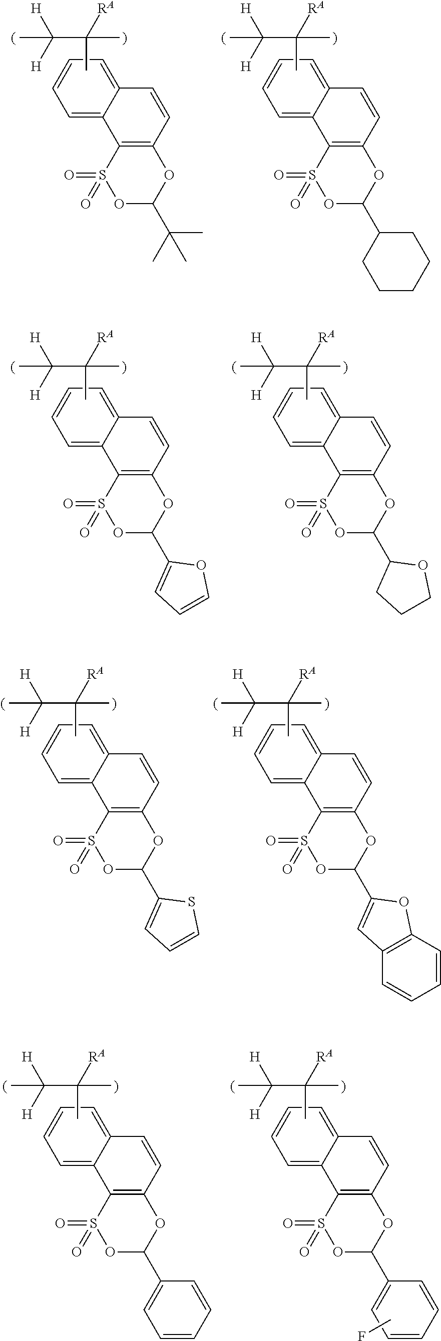

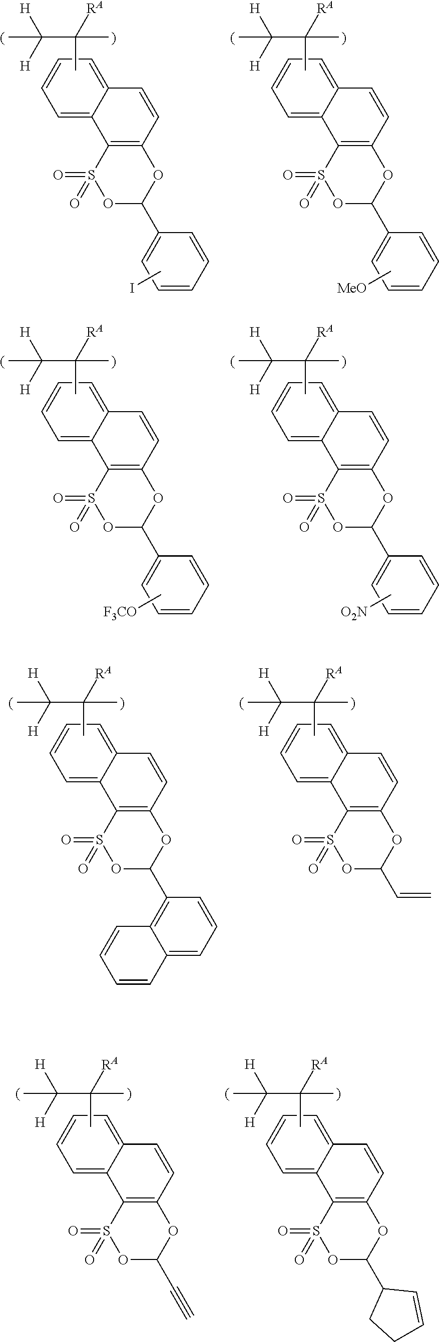

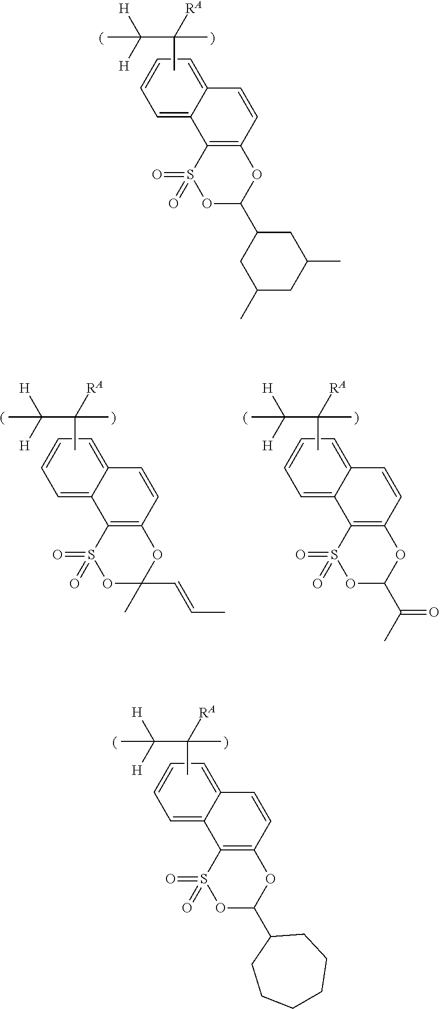

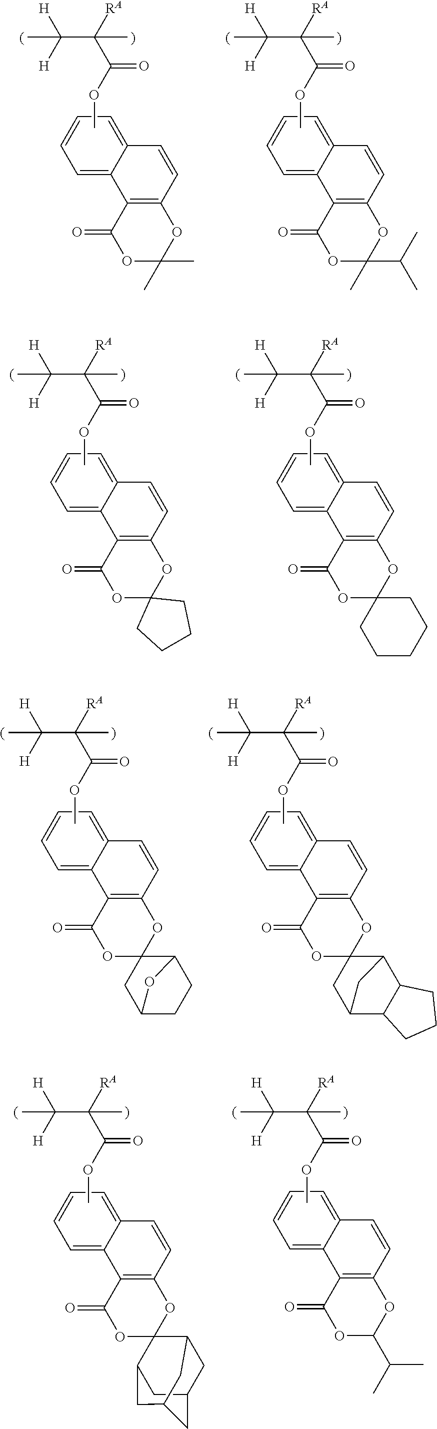

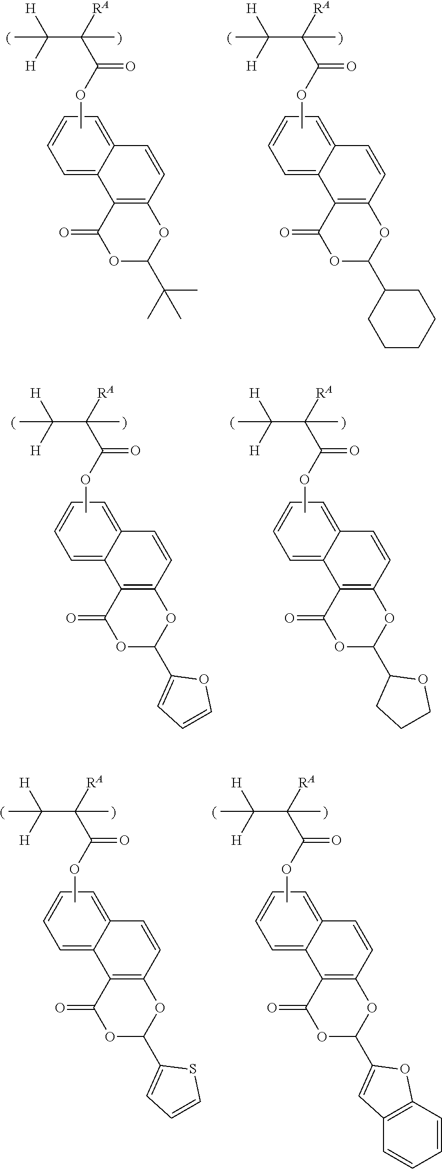

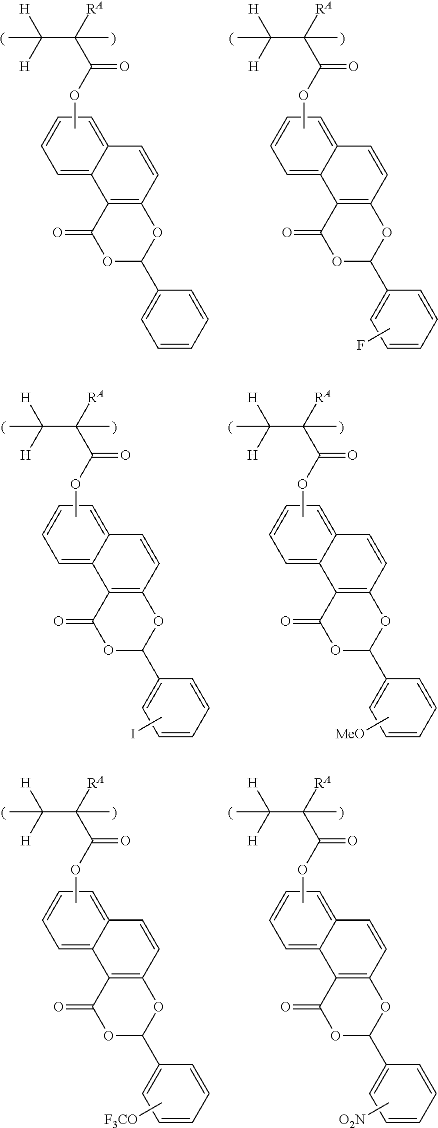

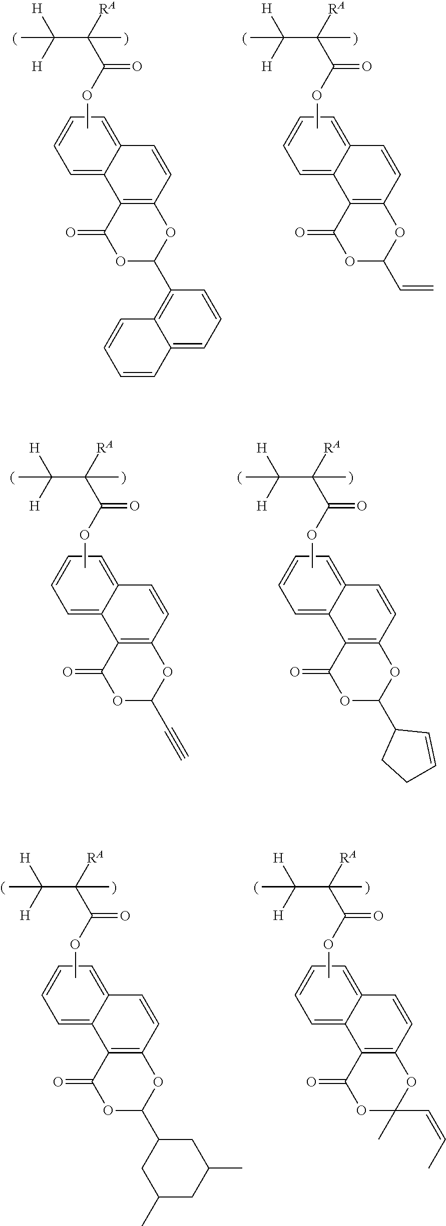

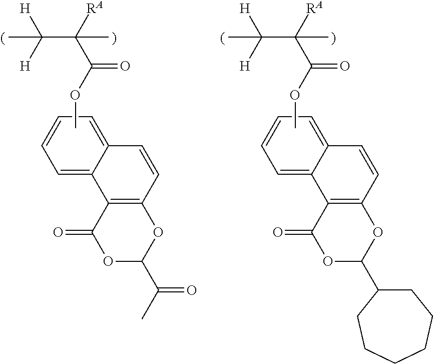

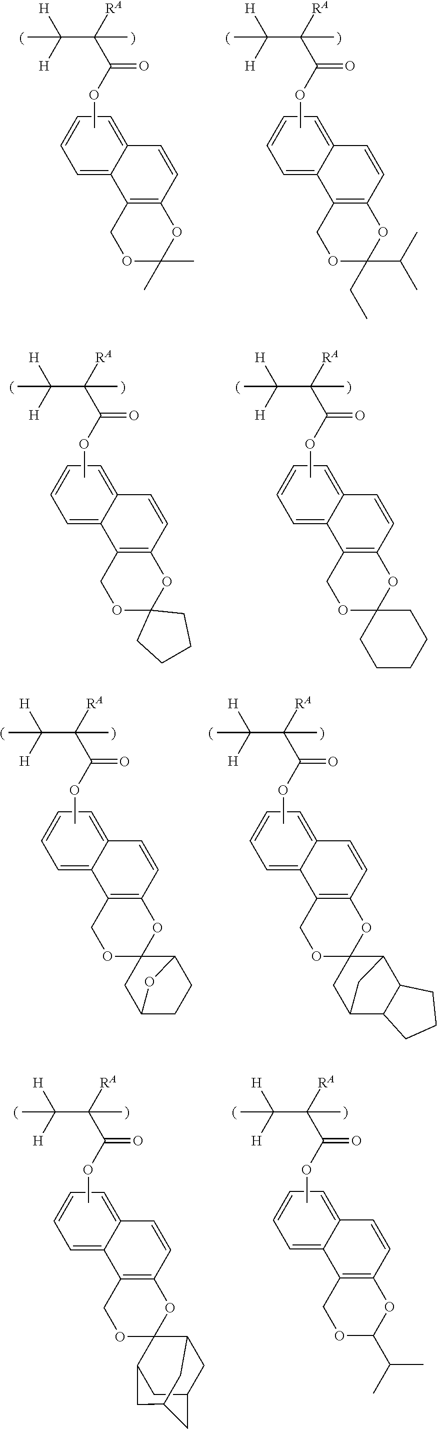

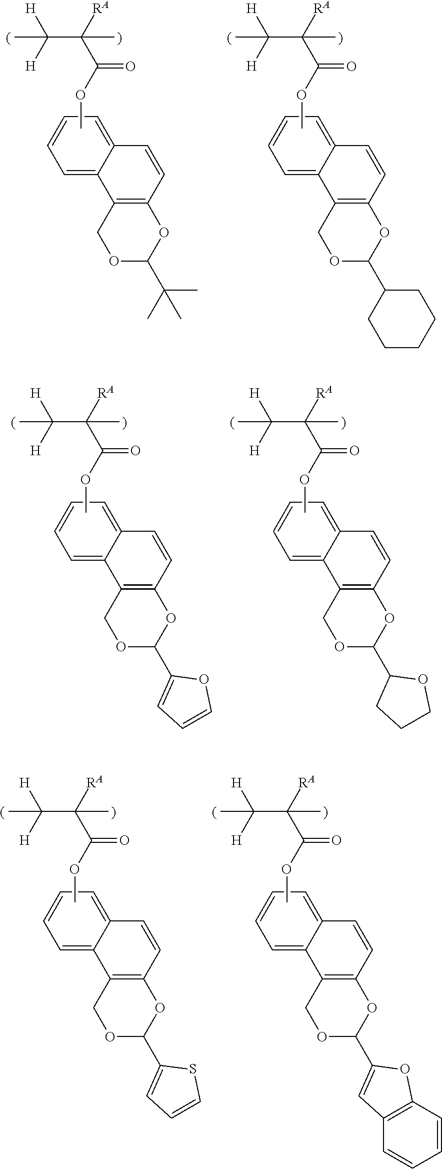

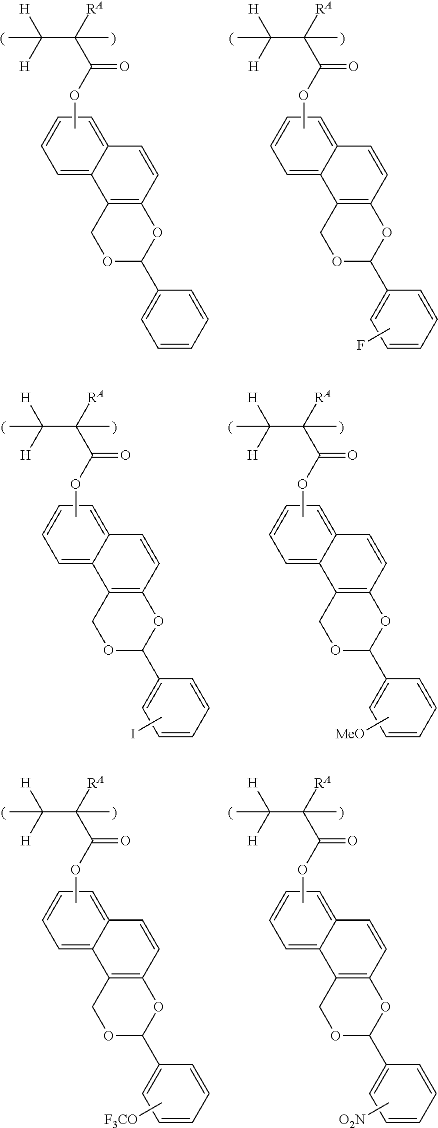

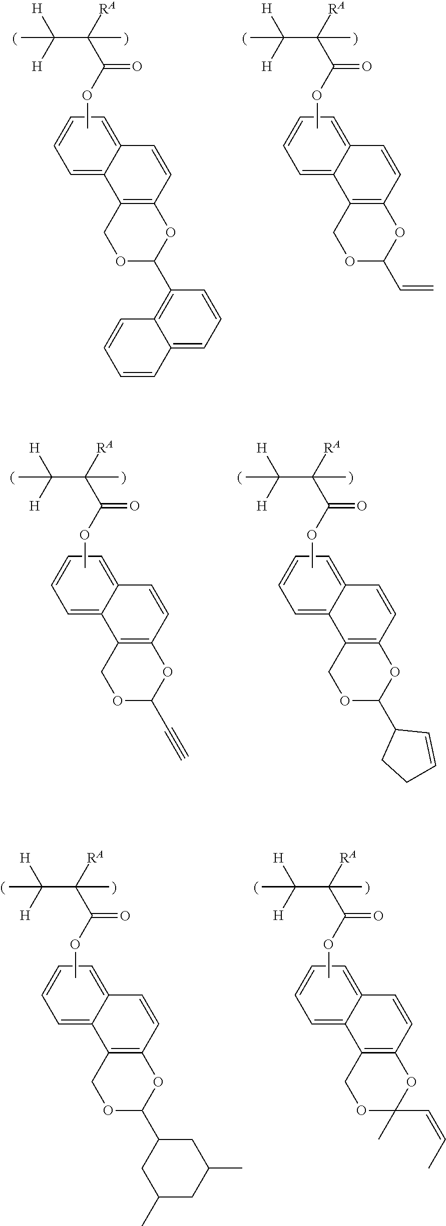

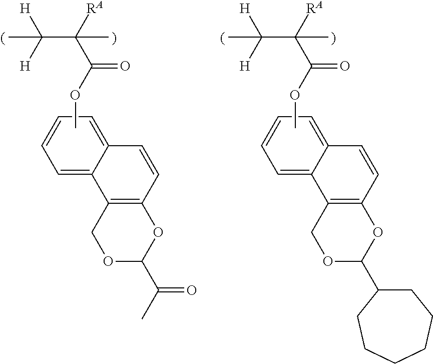

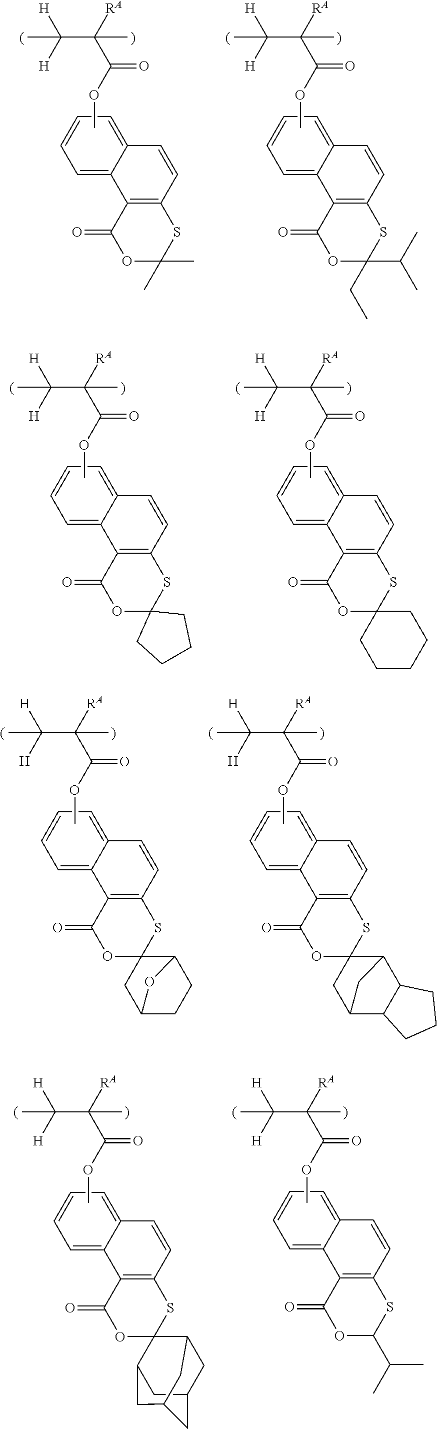

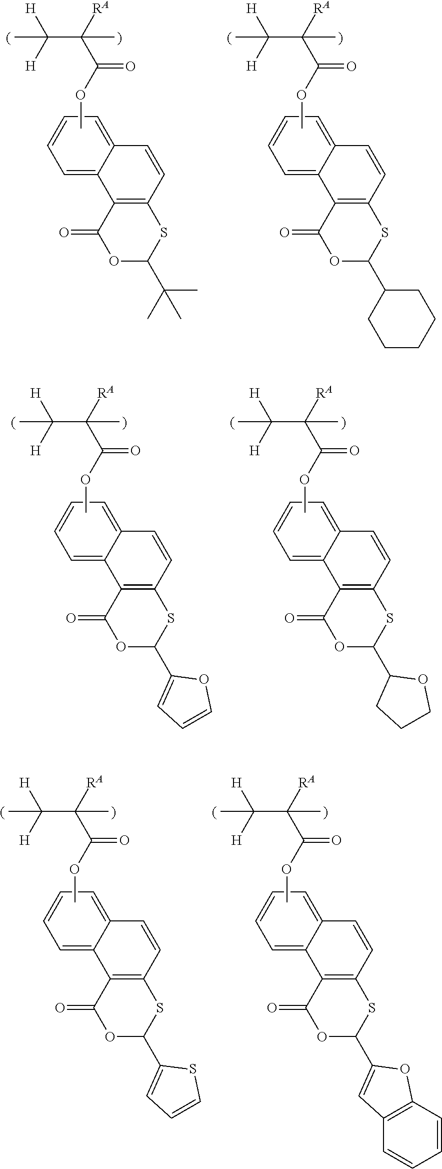

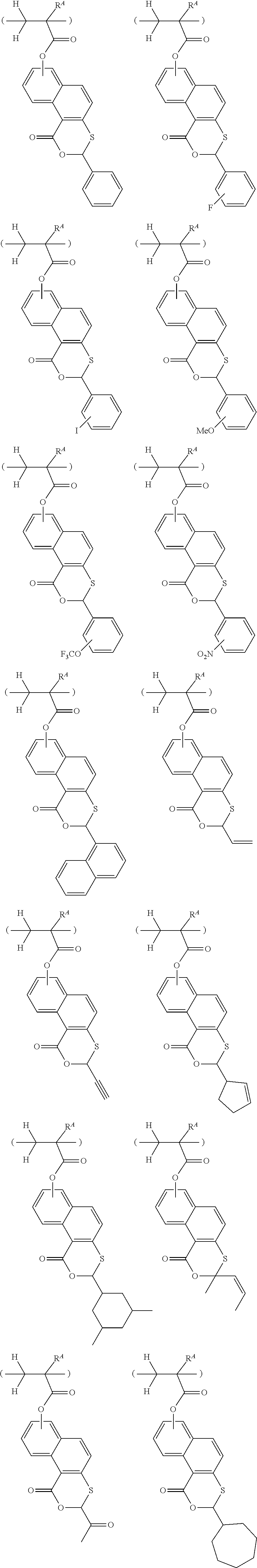

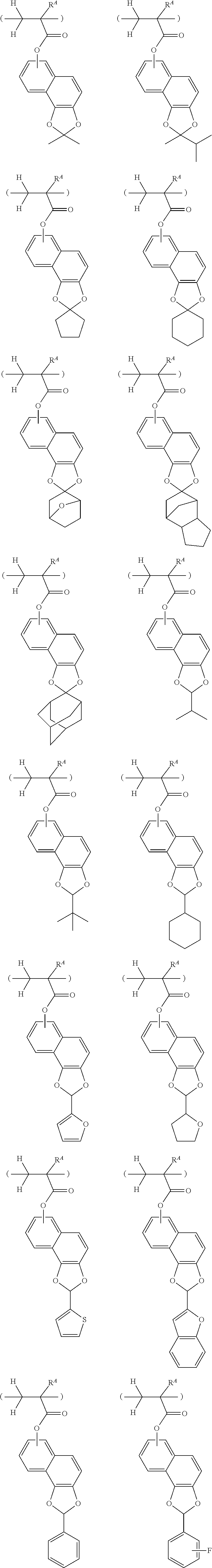

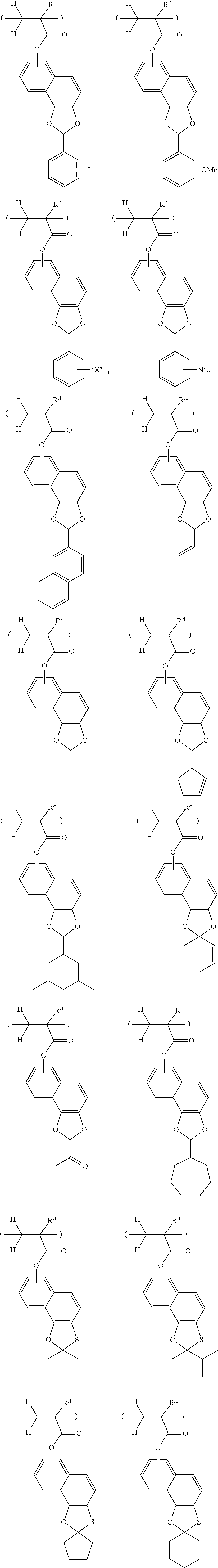

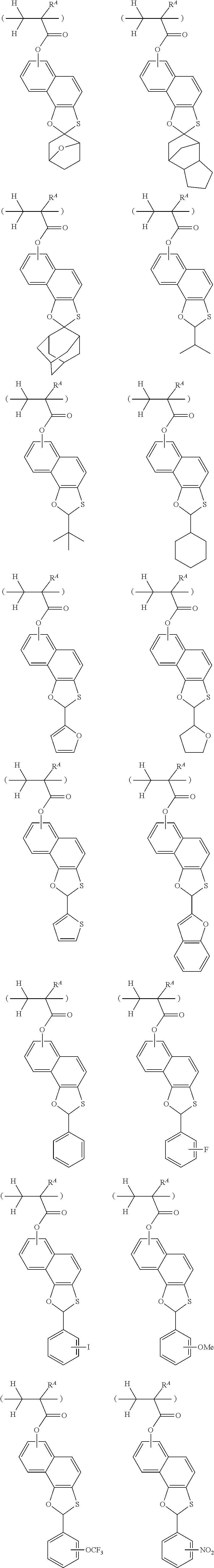

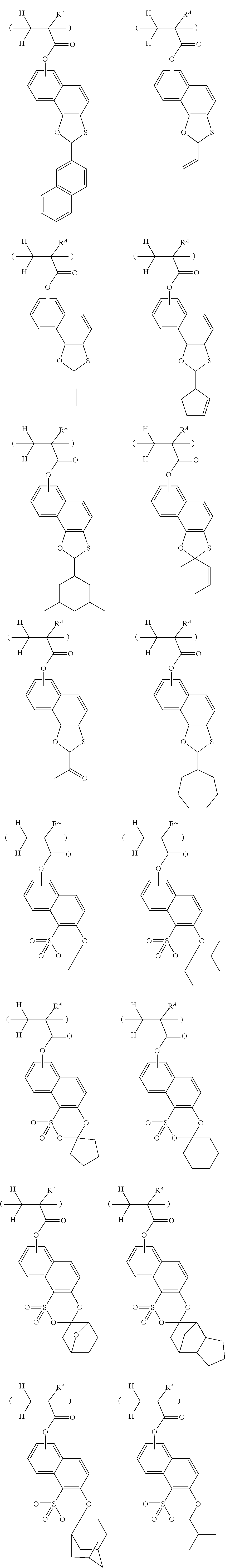

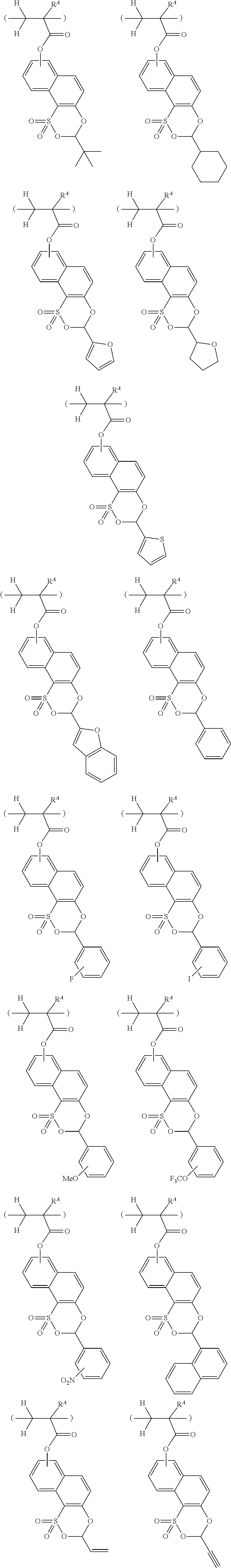

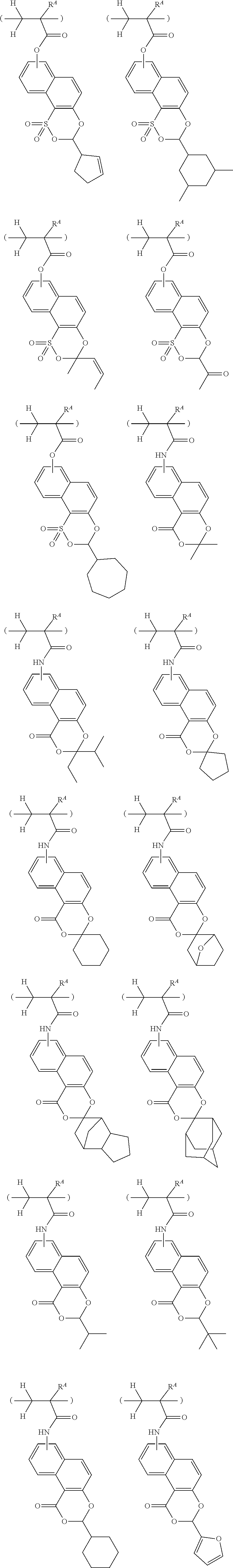

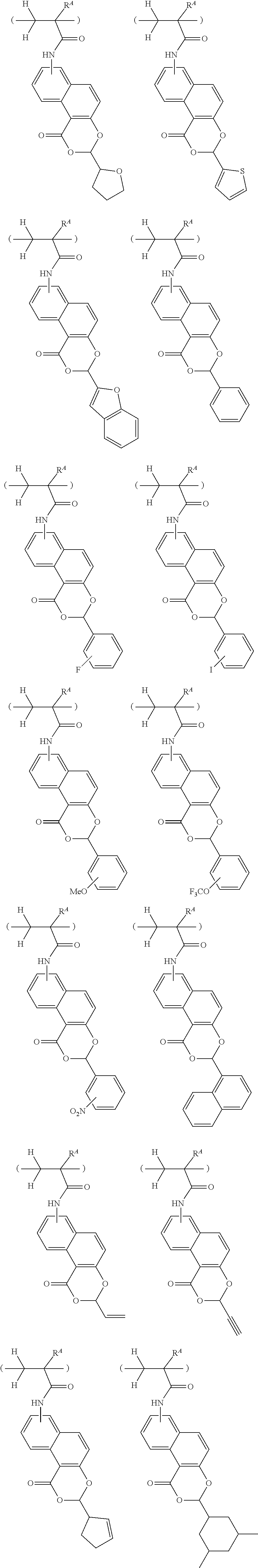

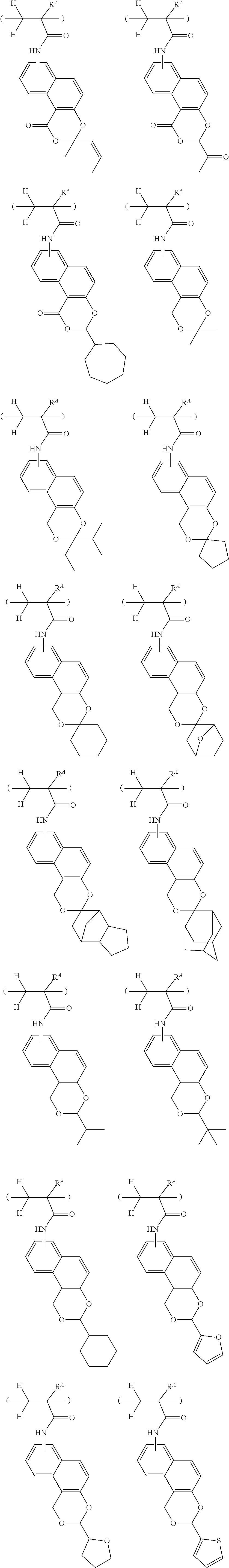

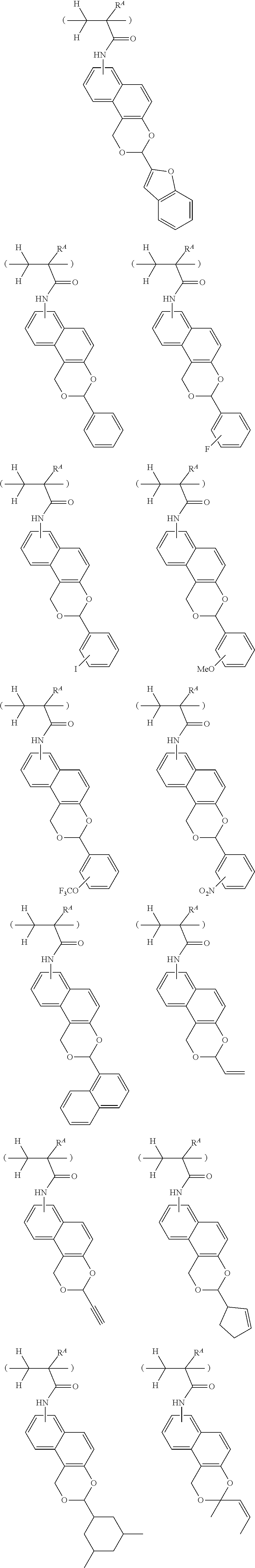

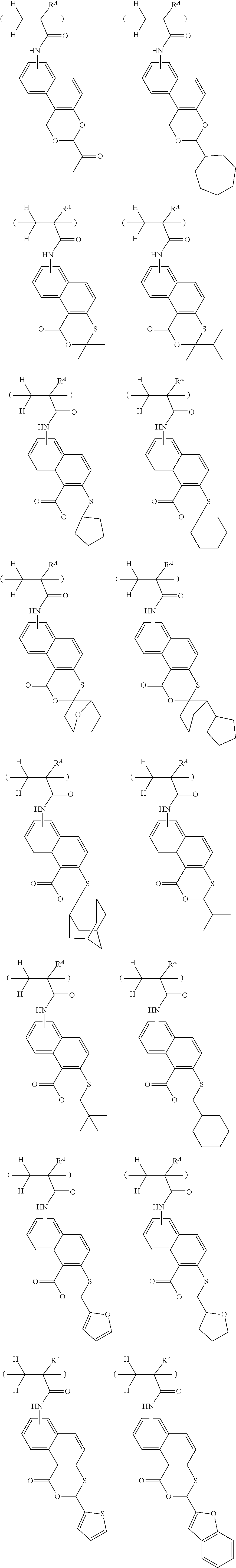

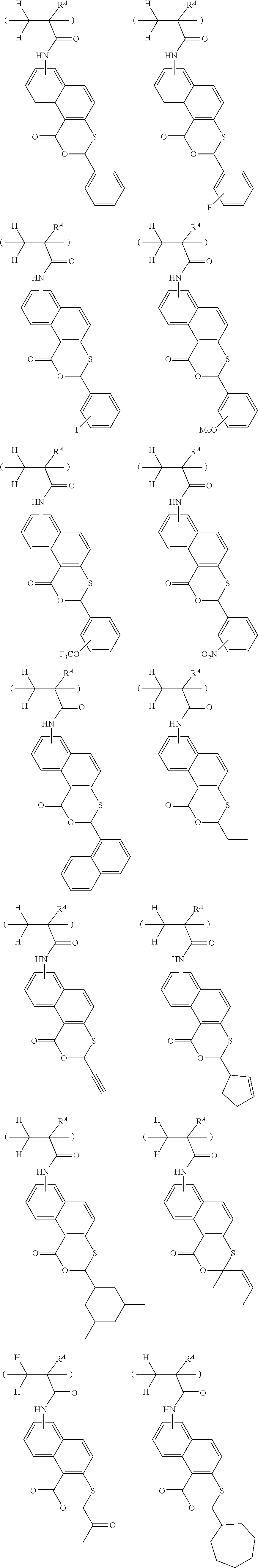

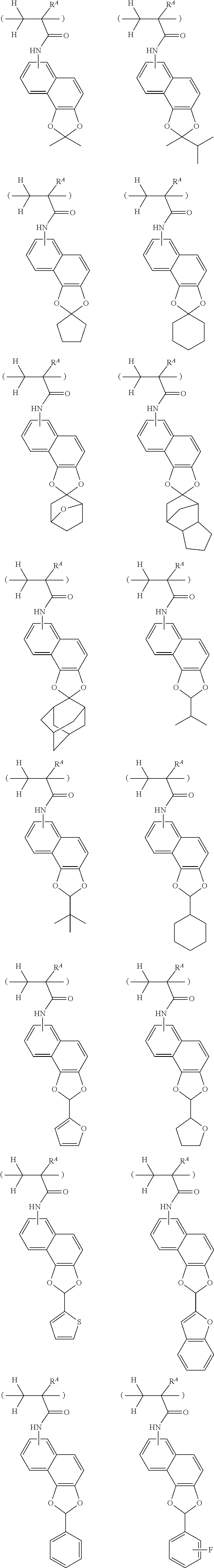

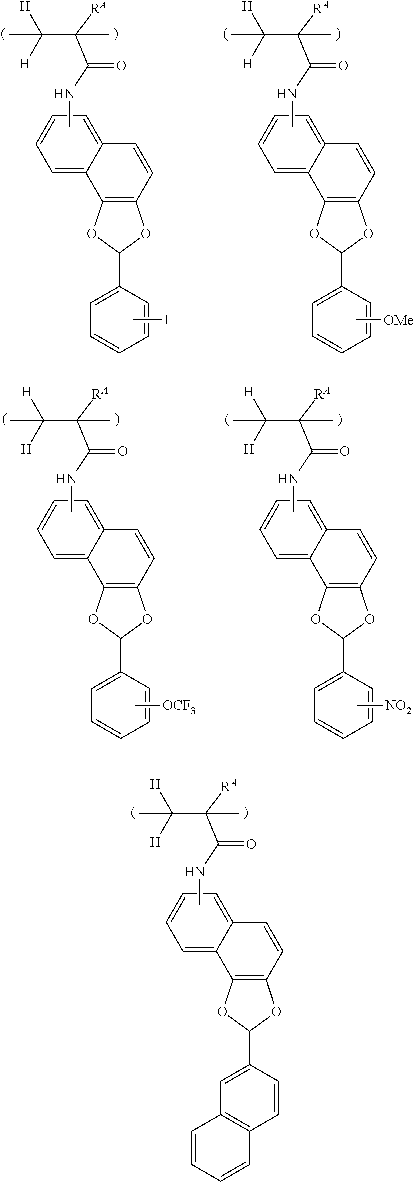

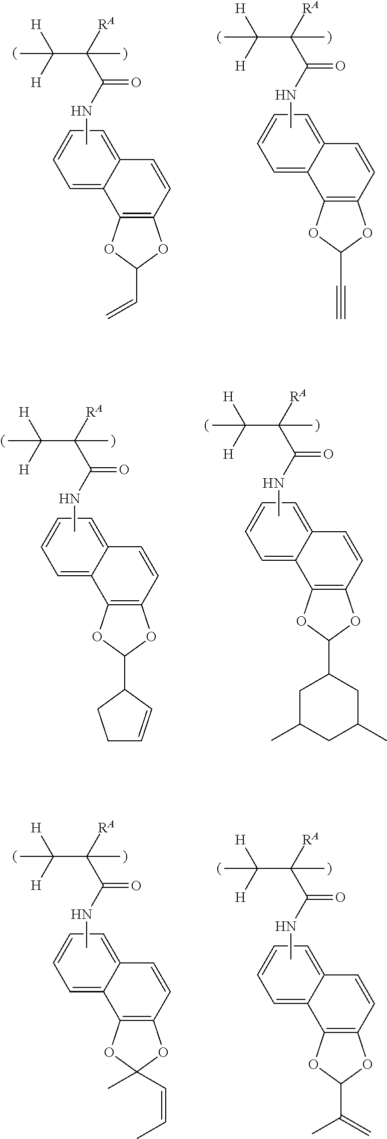

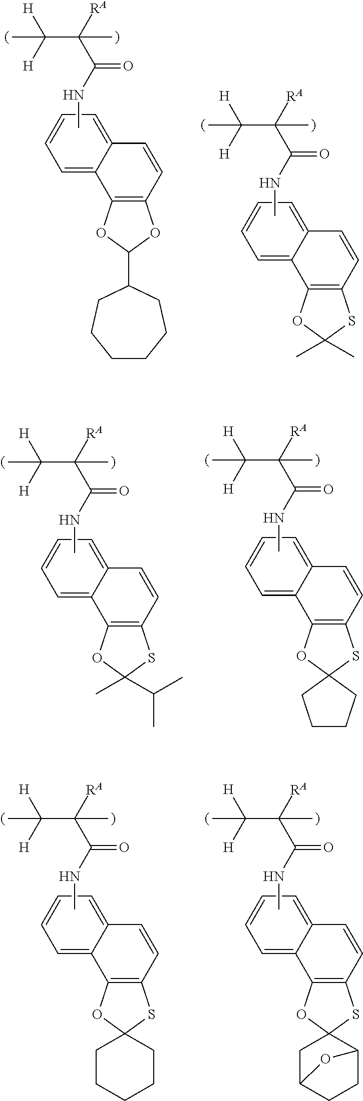

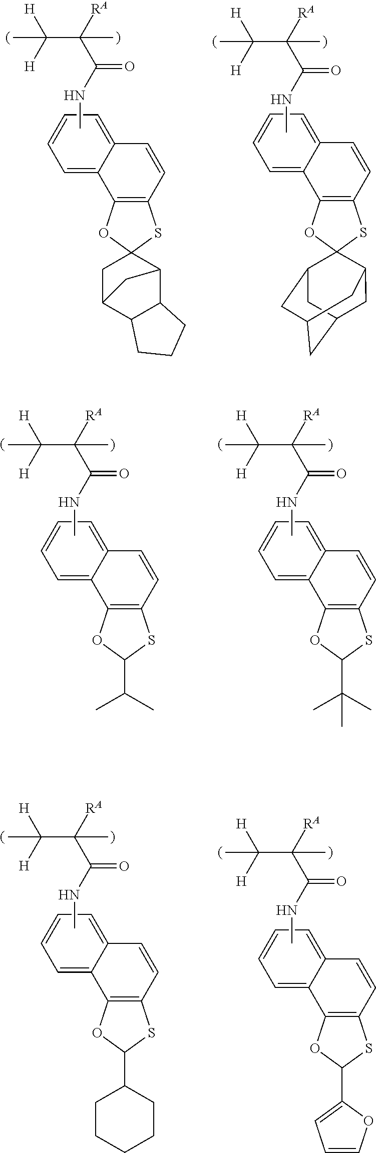

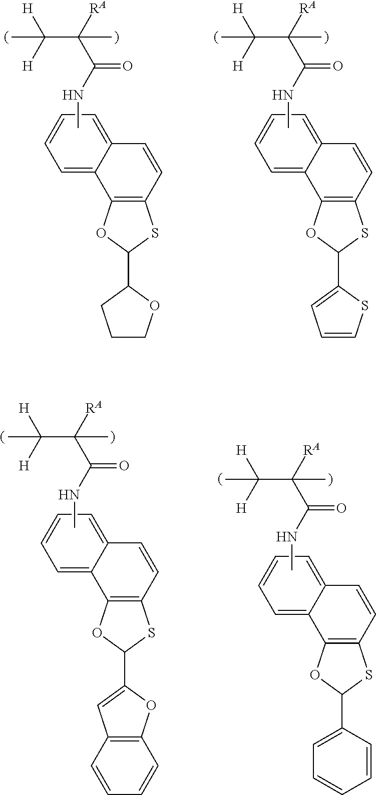

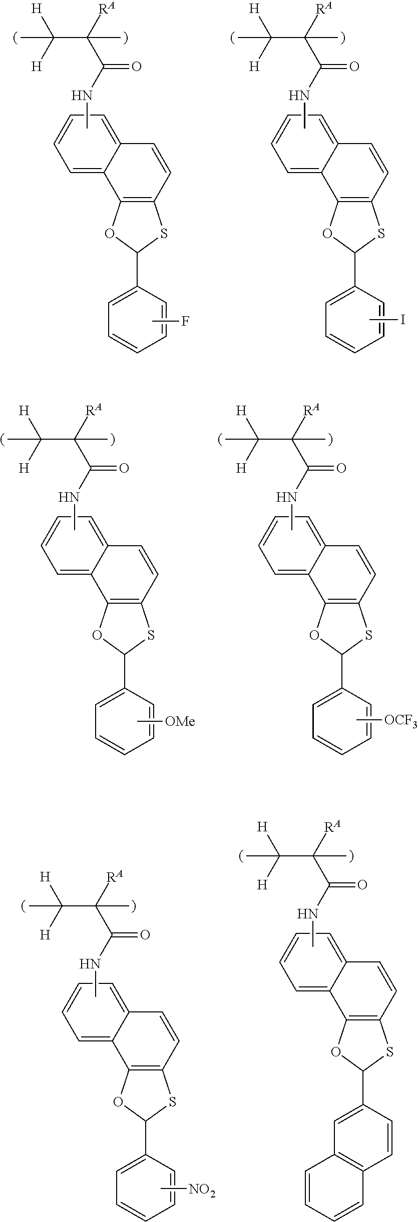

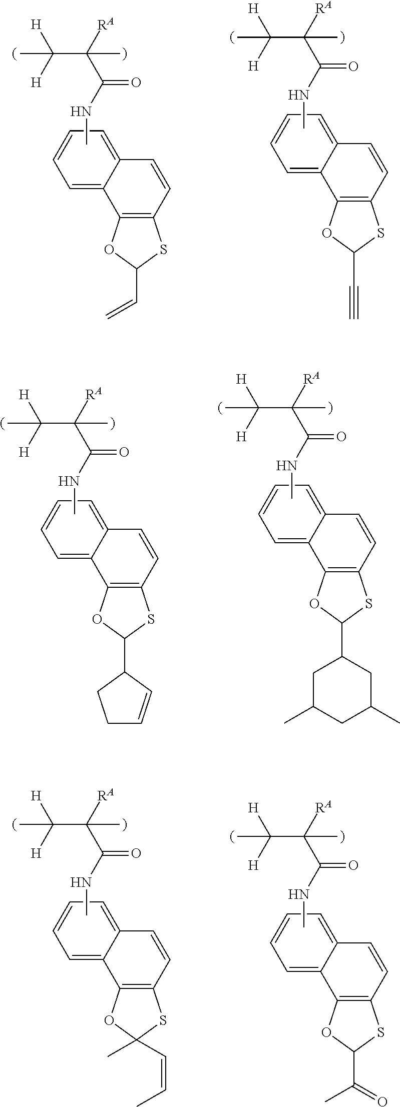

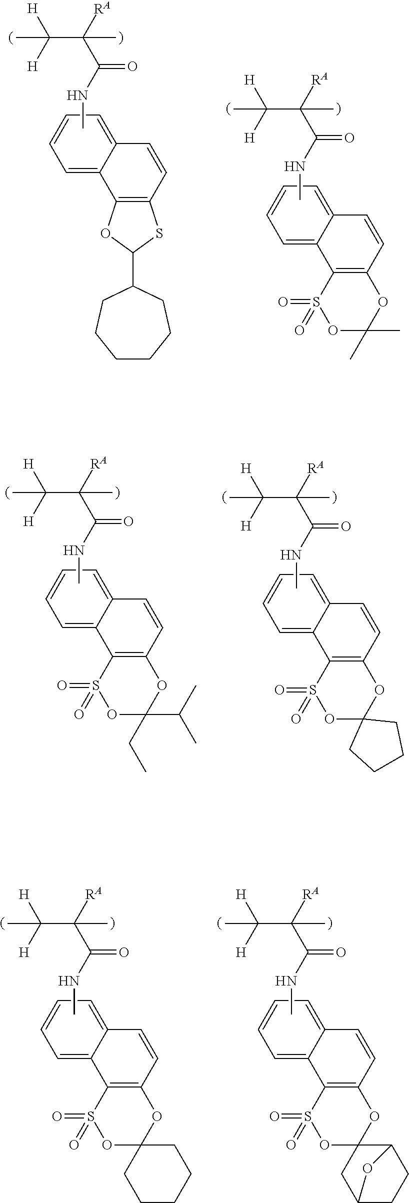

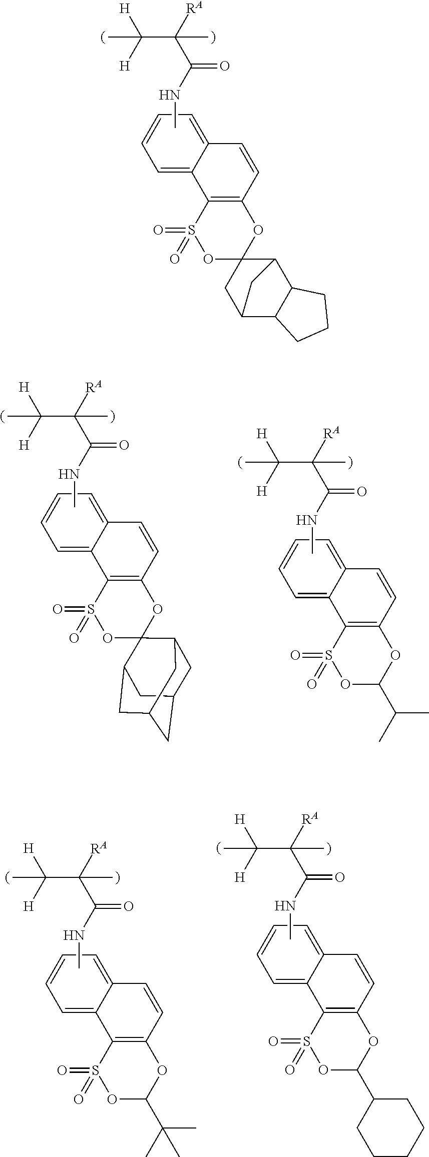

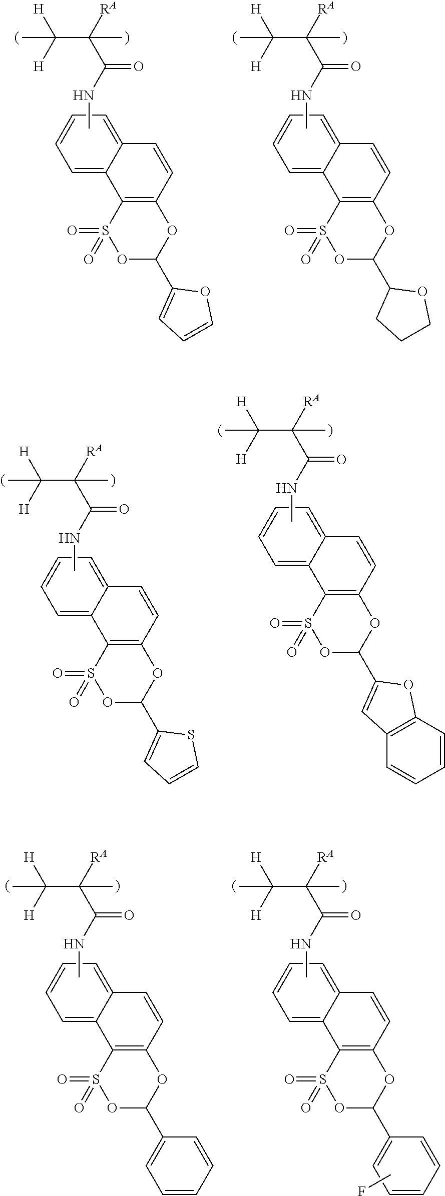

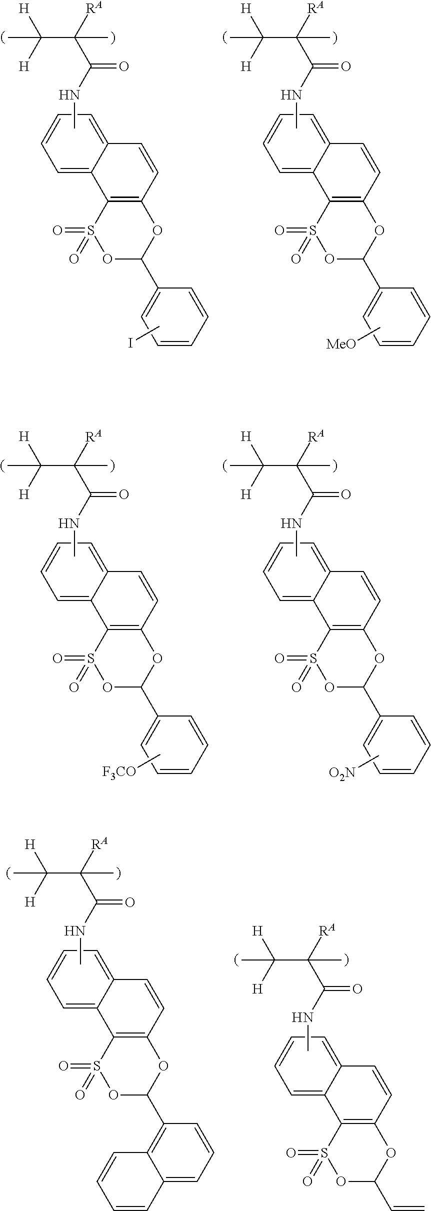

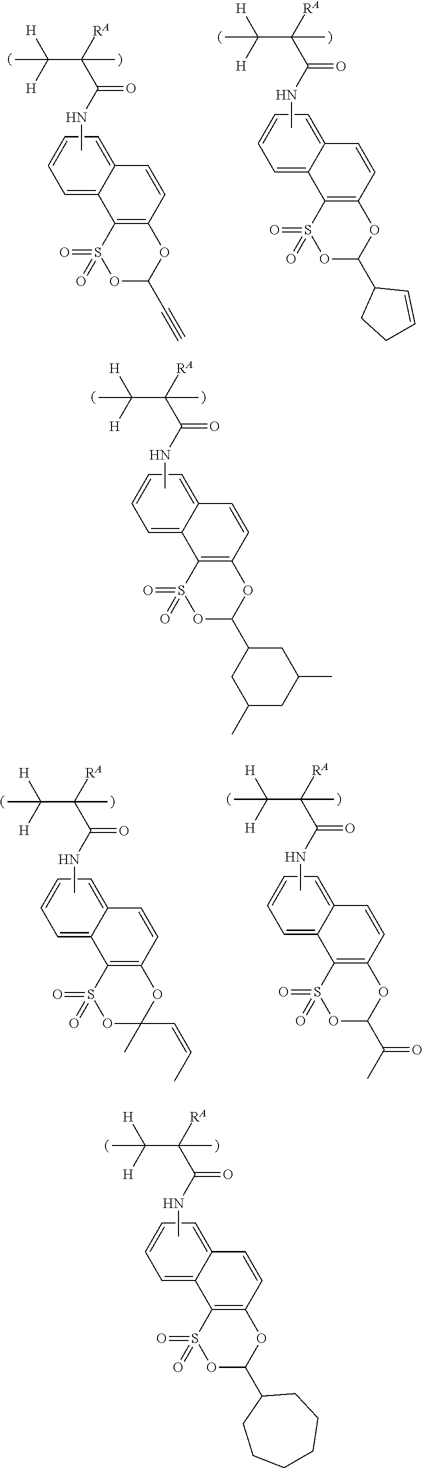

It is preferable that the base polymer further contains a repeating unit represented by the following general formula (b1) (hereinafter also referred to as “repeating unit-b1”) and/or a repeating unit represented by the following general formula (b2) (hereinafter also referred to as “repeating unit-b2”). That is, it is also preferable that the base polymer further contains a repeating unit represented by either one or both of the following general formulae (b1) and (b2).

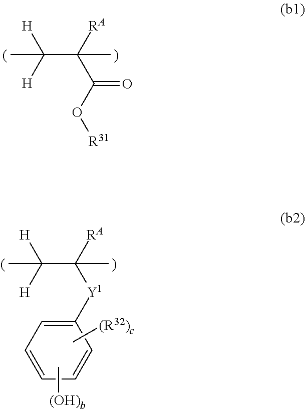

wherein R^{A} each independently represents a hydrogen atom, a fluorine atom, a methyl group, or a trifluoromethyl group;
Y¹ represents a single bond or *—C(═O)—O—; * represents an attachment point to a main chain carbon atom;
R³¹ represents a hydrogen atom, or a group having 1 to 20 carbon atoms and containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic acid anhydride (—C(═O)—O—C(═O)—);
R³² represents a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “c” is 2 or more, each R³² may be the same as or different from each other;
“b” represents an integer from 1 to 4; and “c” represents an integer from 0 to 4; however, 1≤b+c≤5.

In the general formulae (b1) and (b2), R^{A} each independently represents a hydrogen atom, a fluorine atom, a methyl group, or a trifluoromethyl group. Y¹ represents a single bond or *—C(═O)—O—. * represents an attachment point to a main chain carbon atom. R³¹ represents a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—). R³² represents a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “b” represents an integer from 1 to 4. “c” represents an integer from 0 to 4. However, 1≤b+c≤5.

Specific examples of the repeating unit-b1 include, but are not limited to, the following. Note that in the following formulae, R^{A} is as defined above.

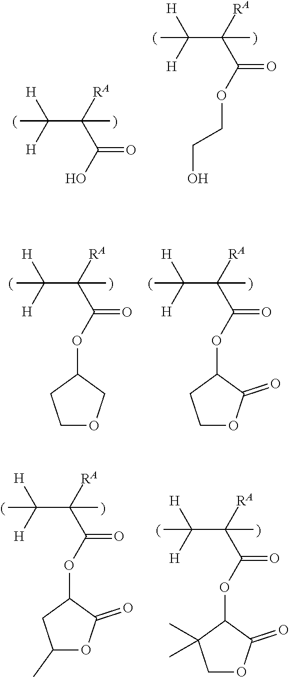

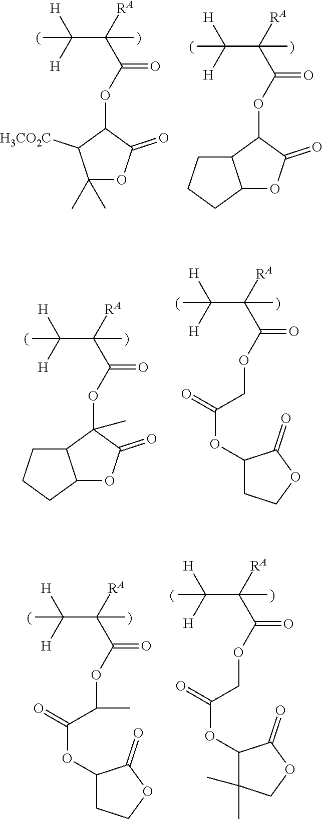

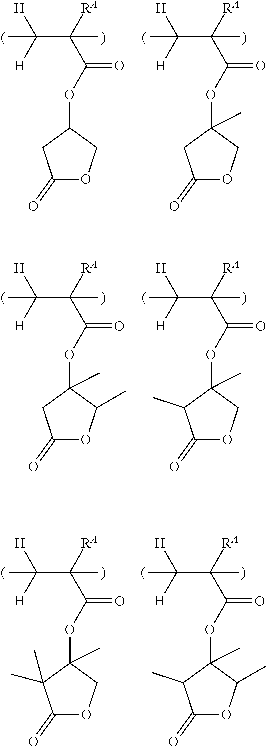

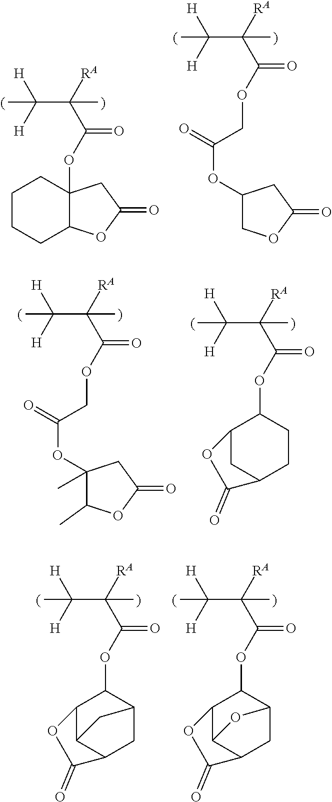

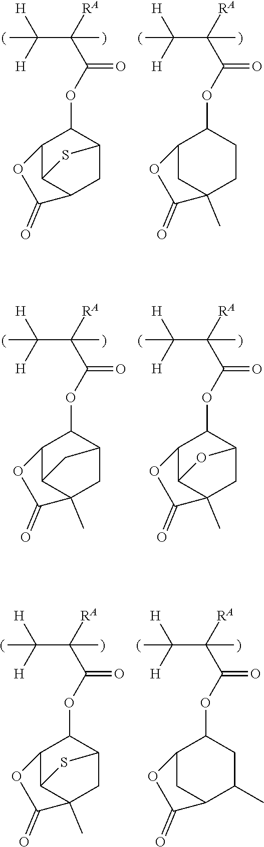

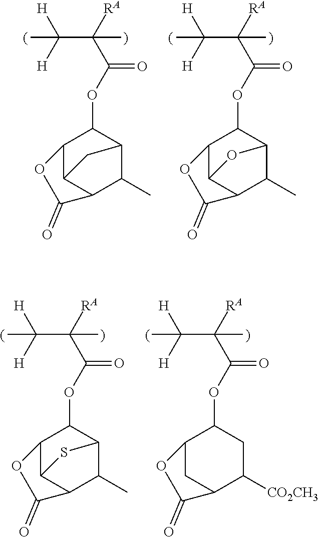

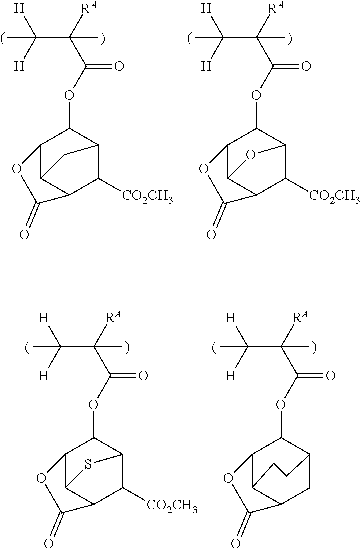

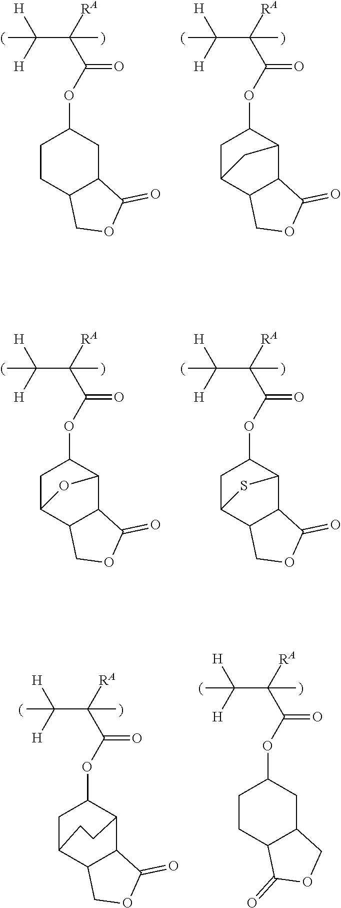

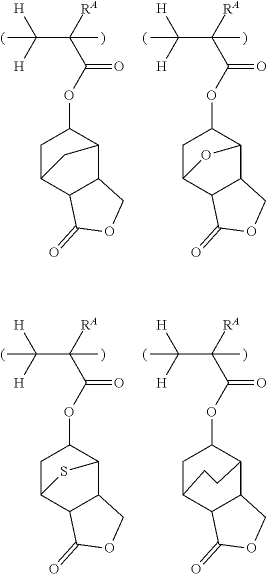

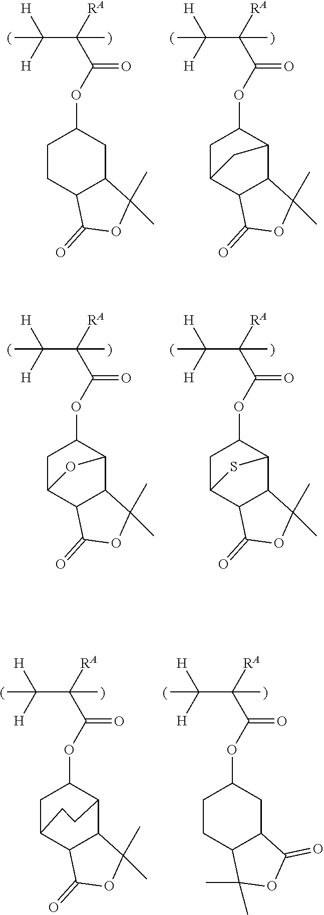

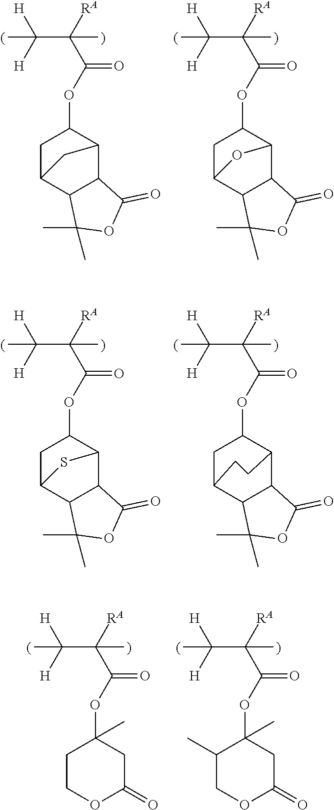

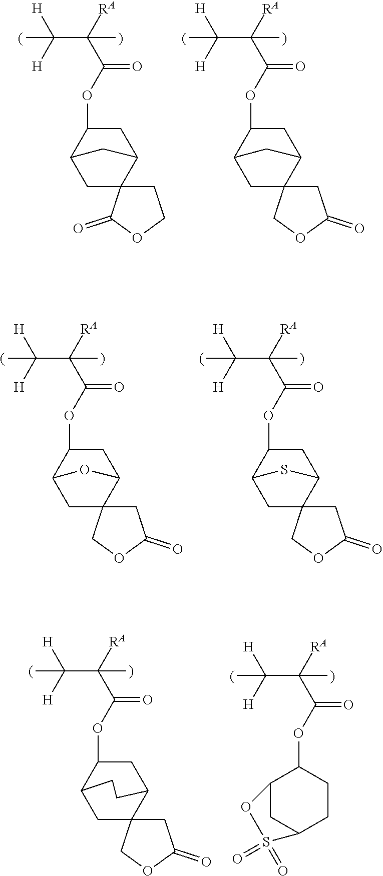

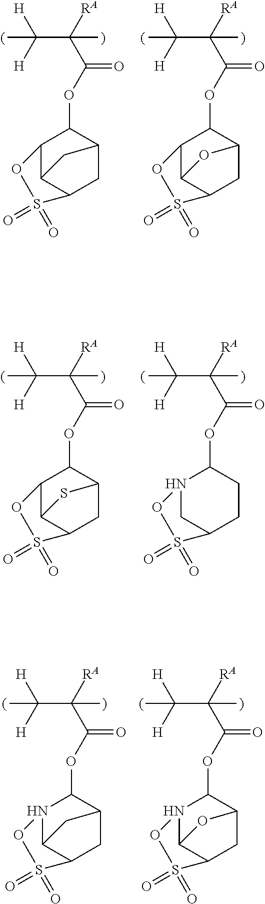

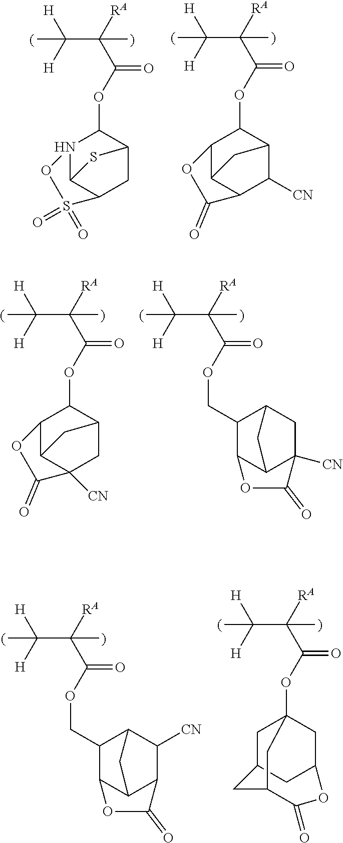

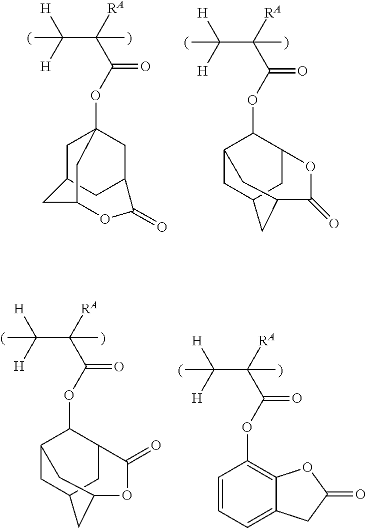

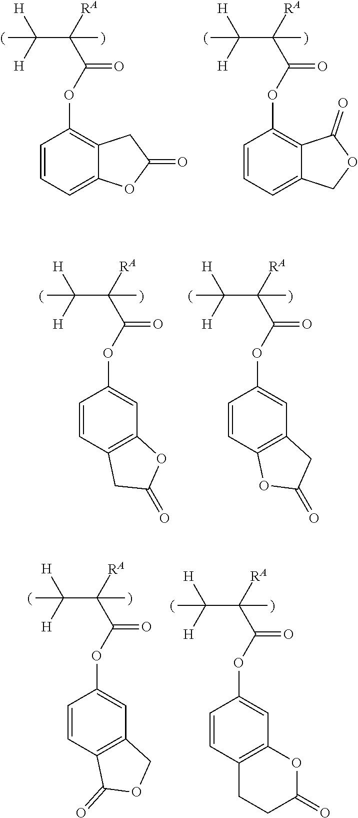

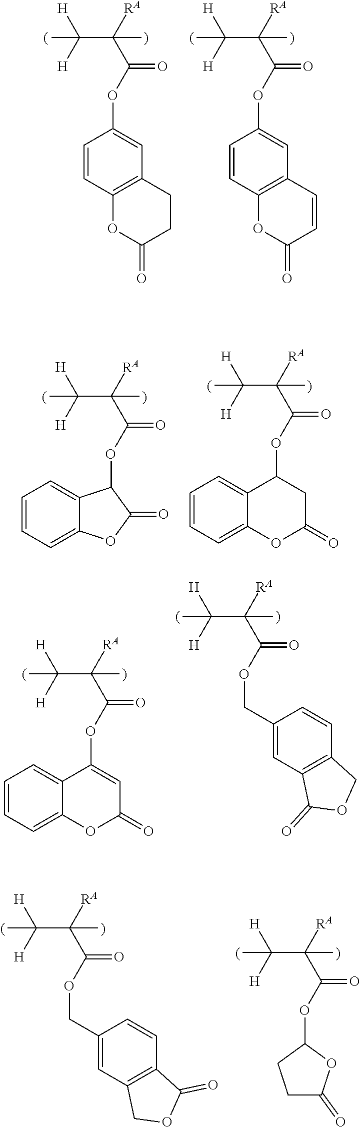

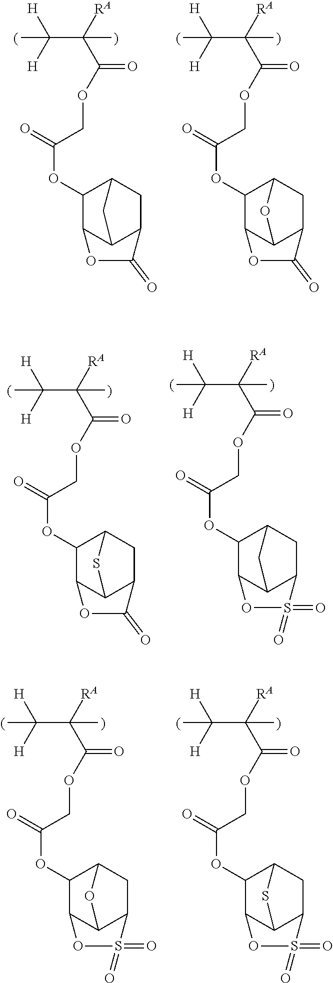

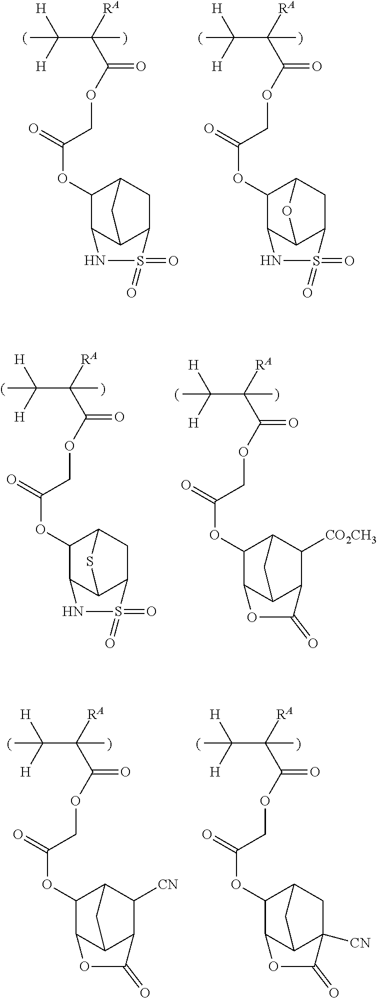

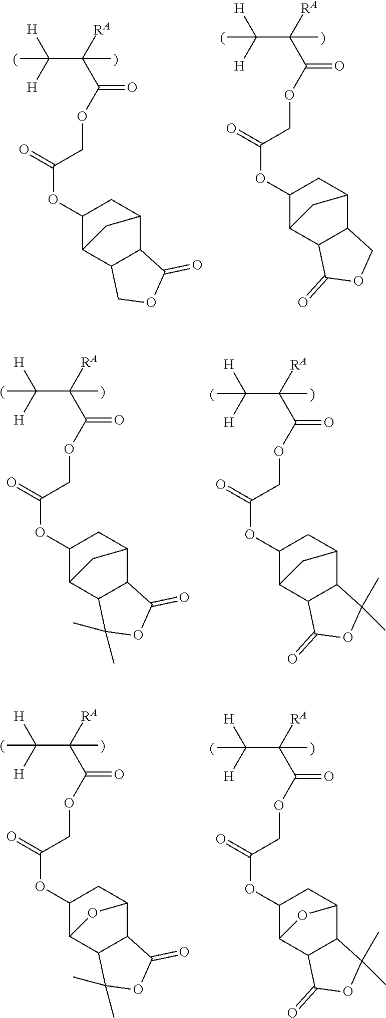

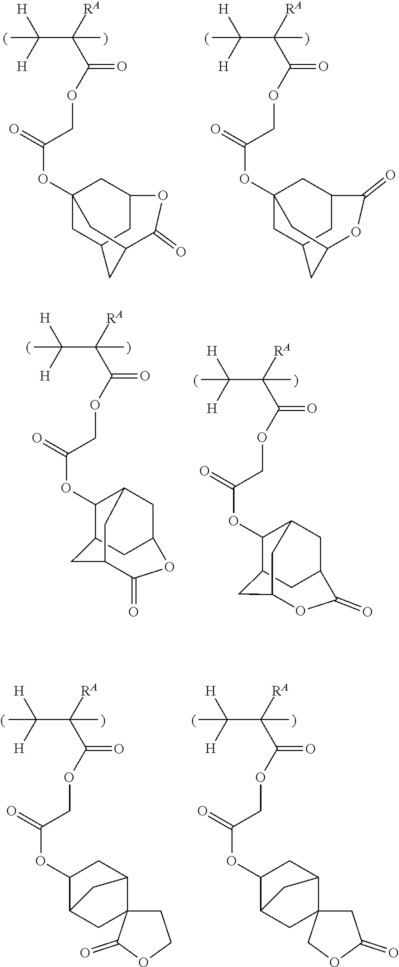

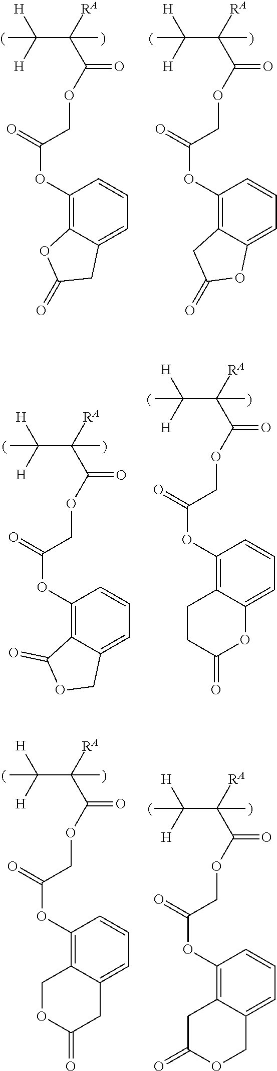

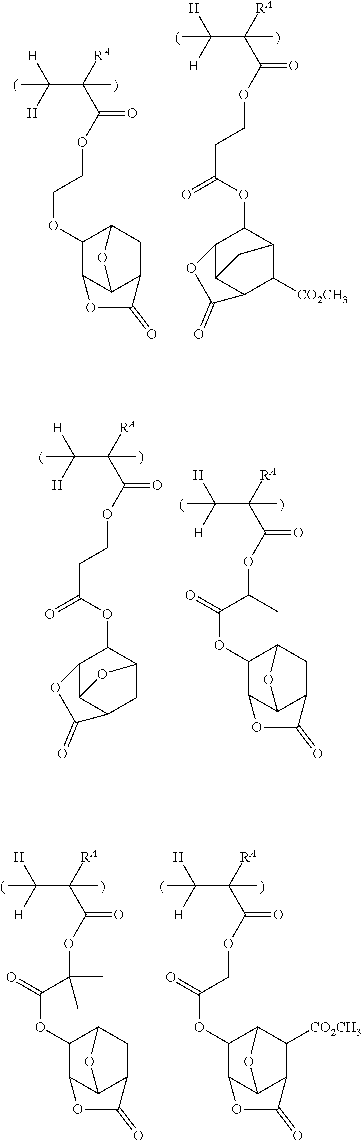

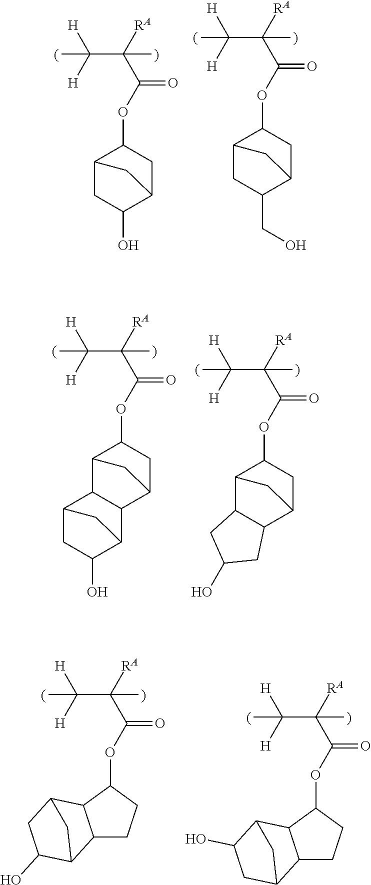

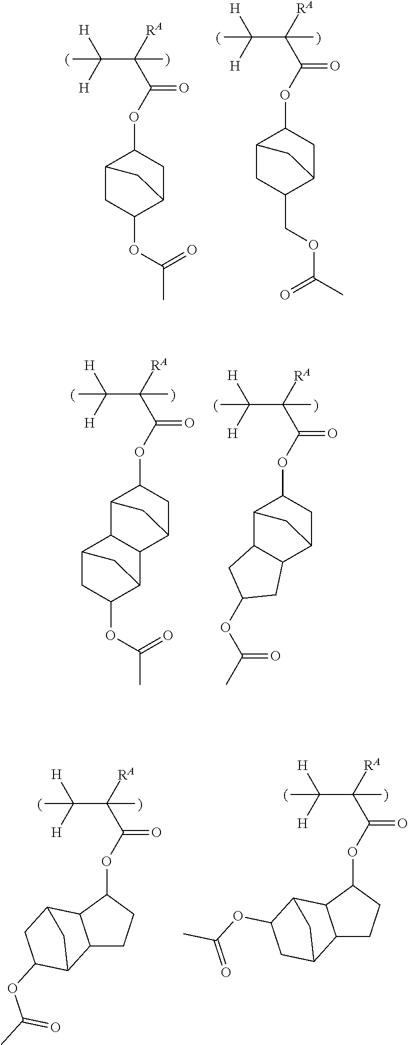

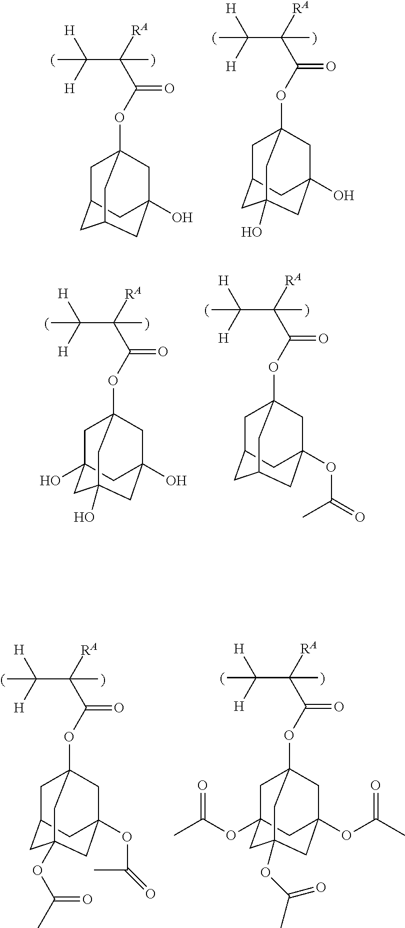

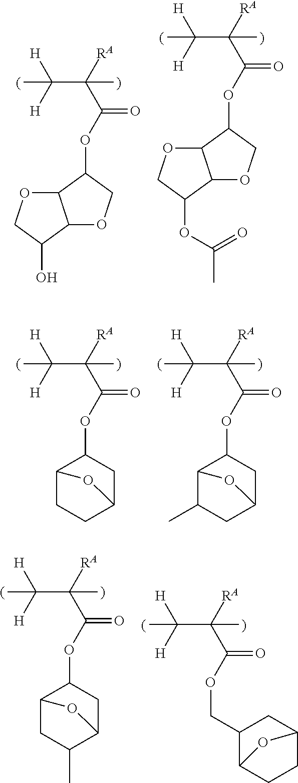

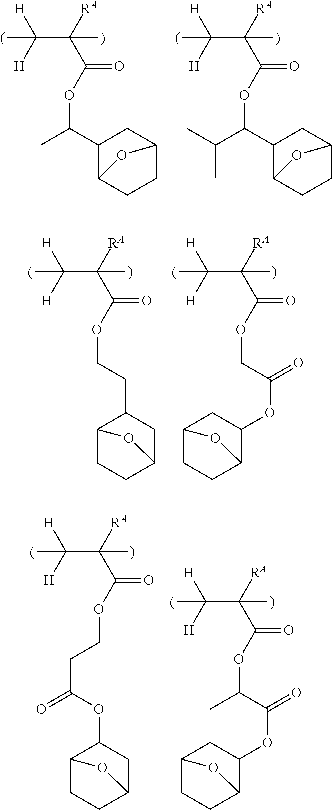

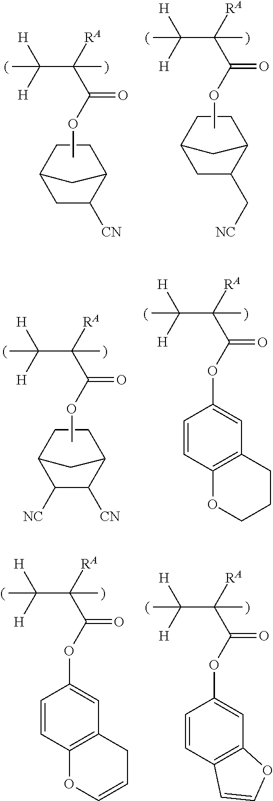

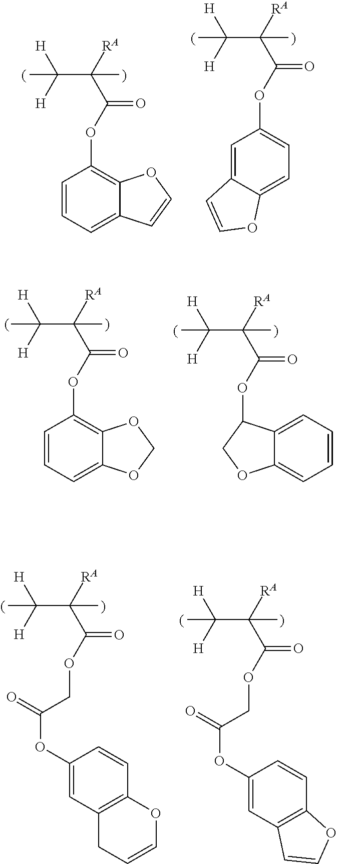

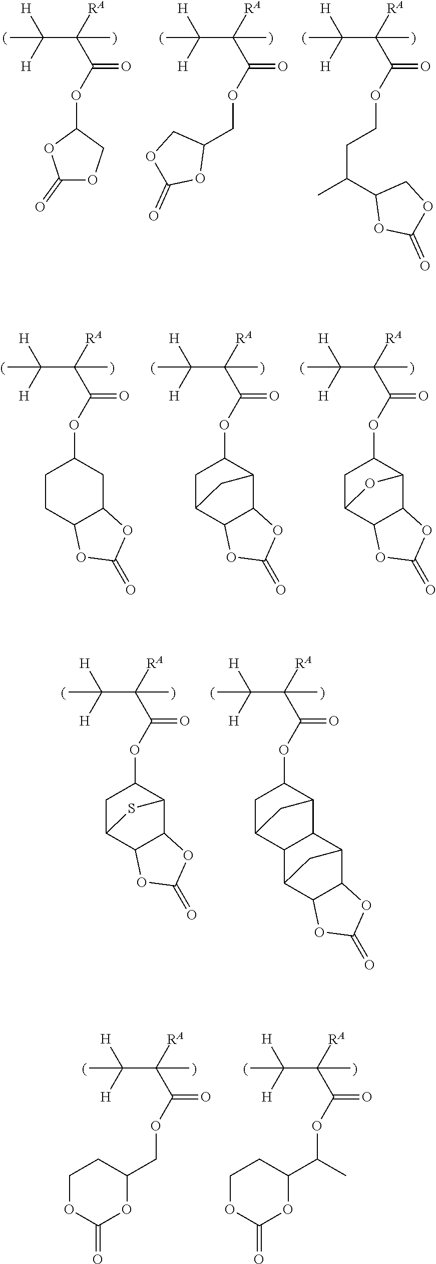

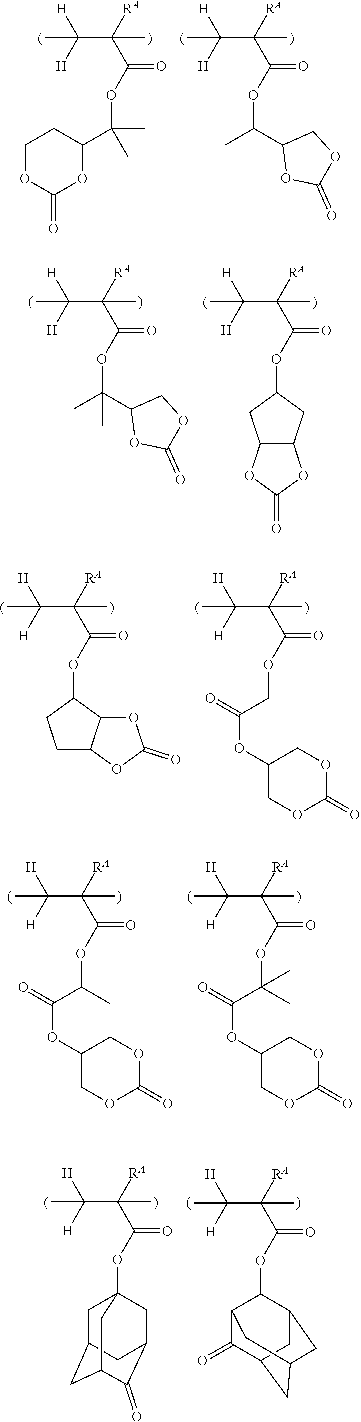

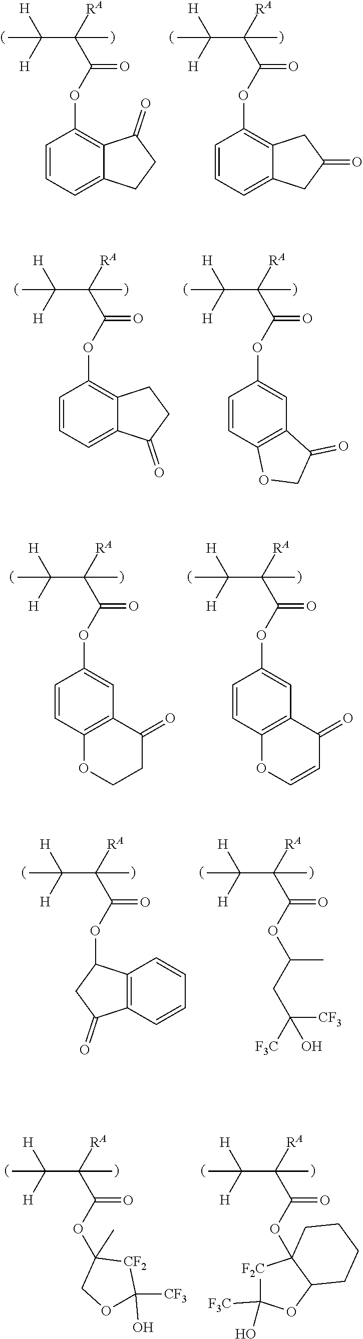

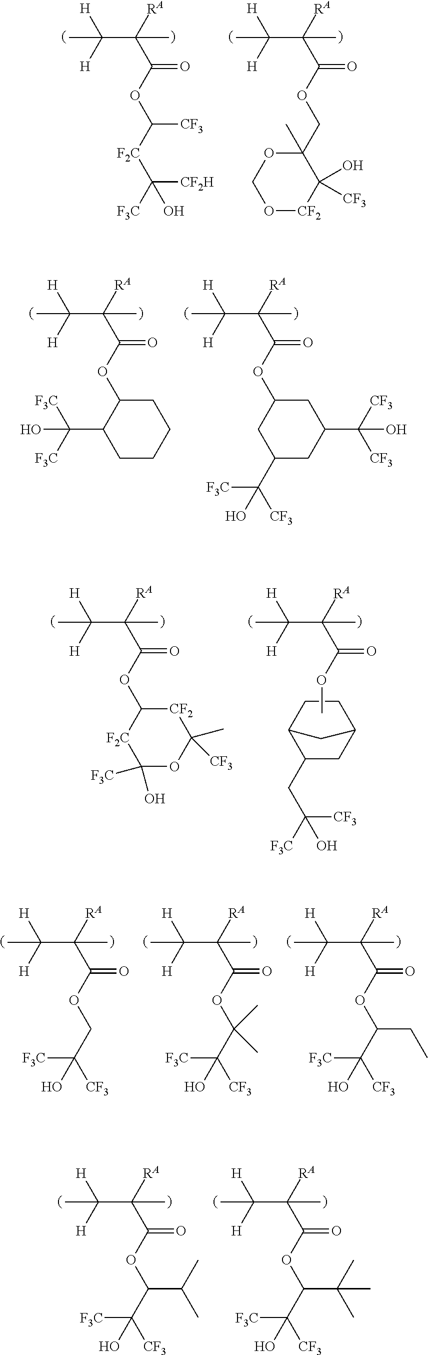

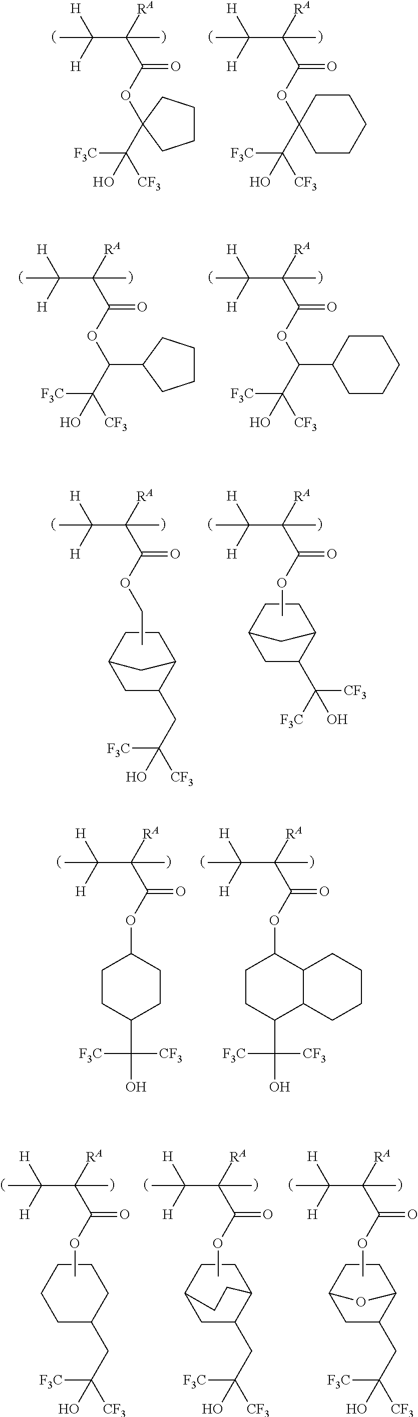

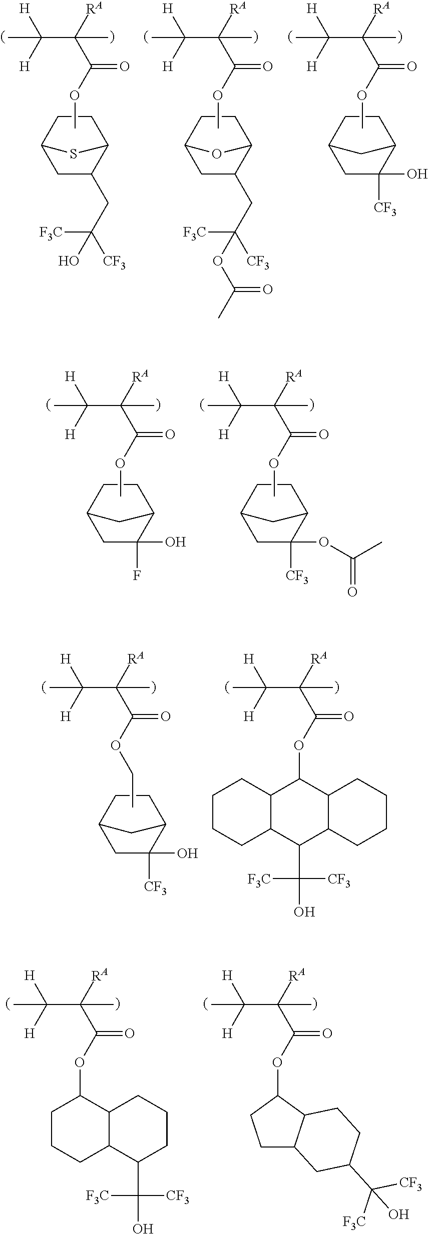

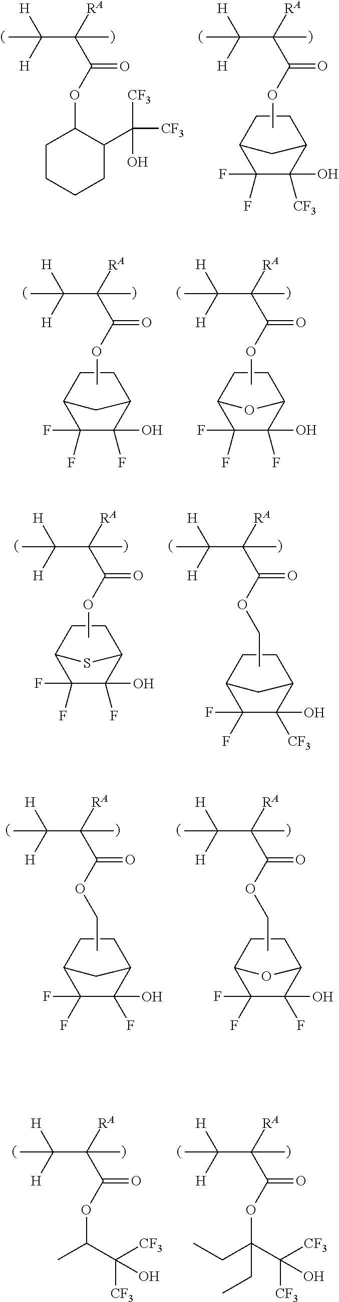

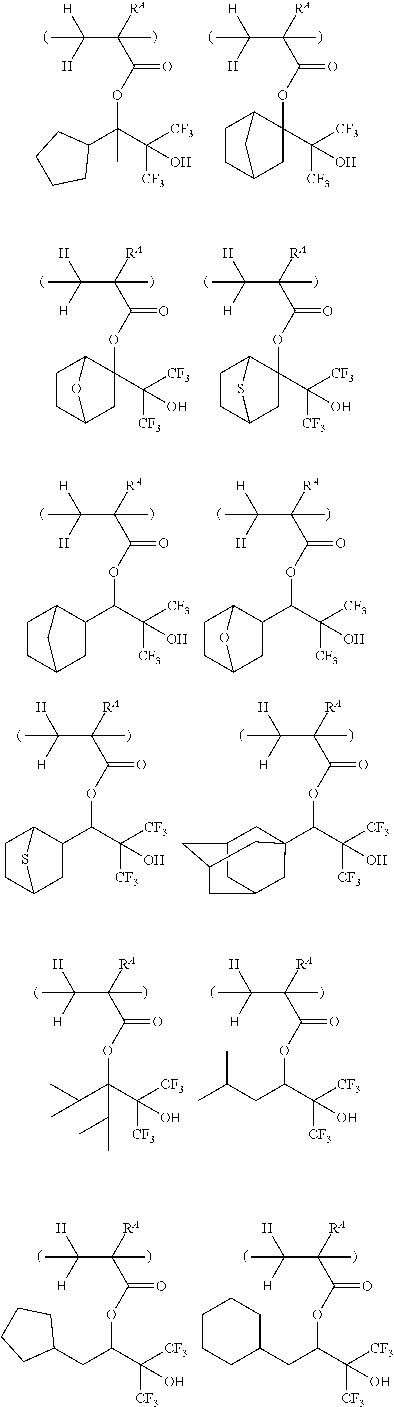

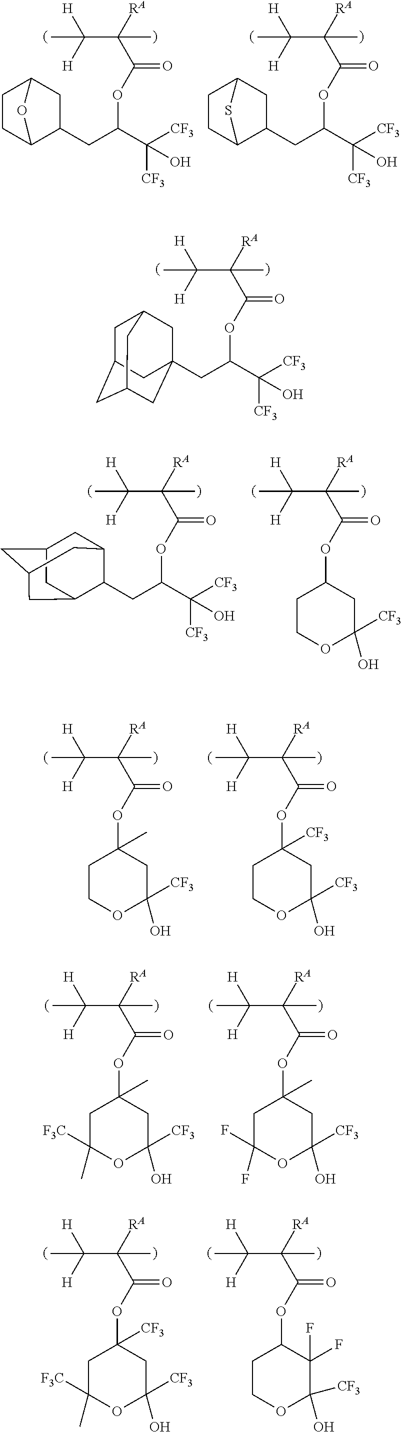

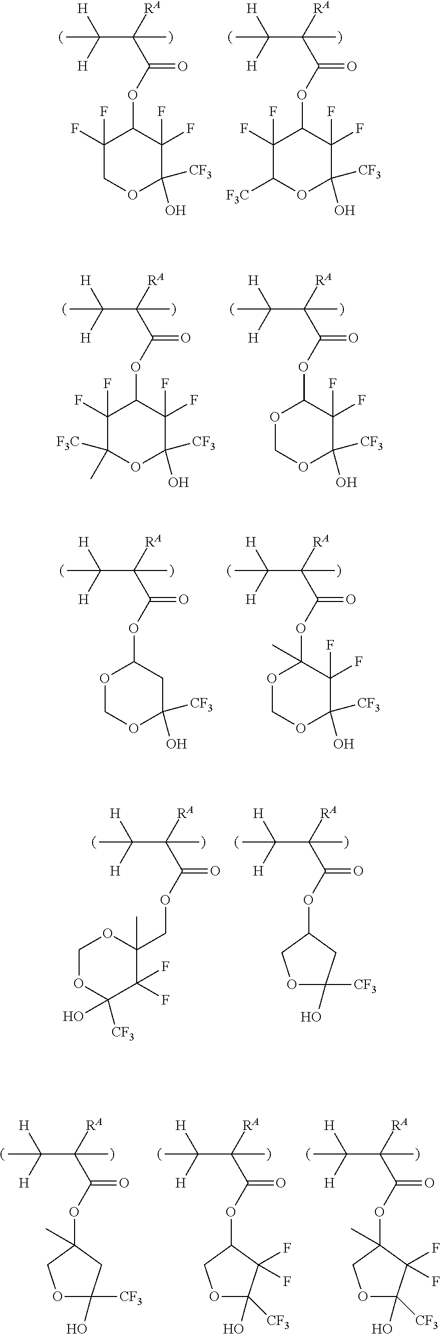

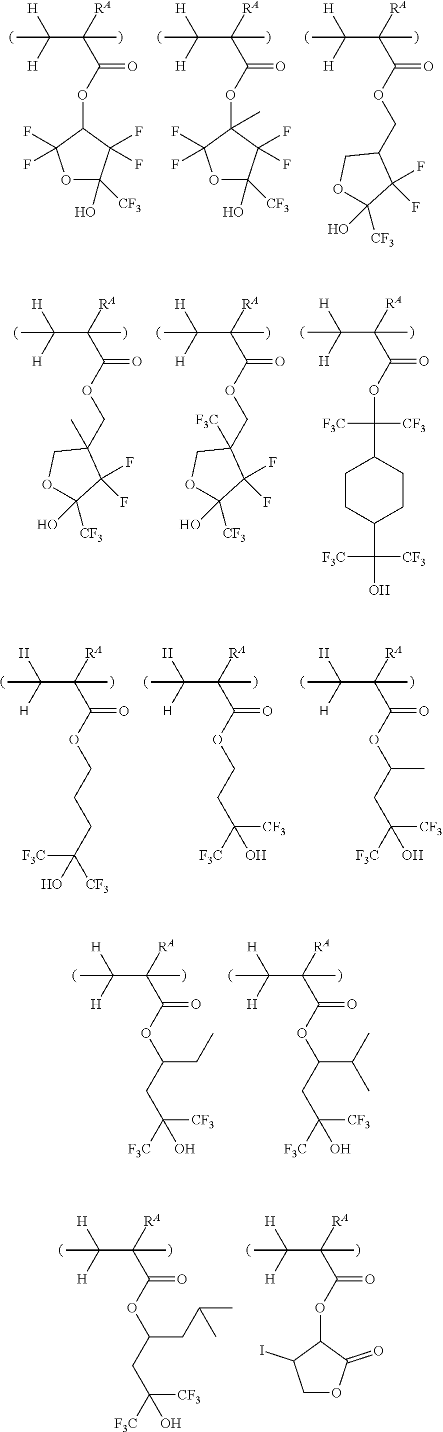

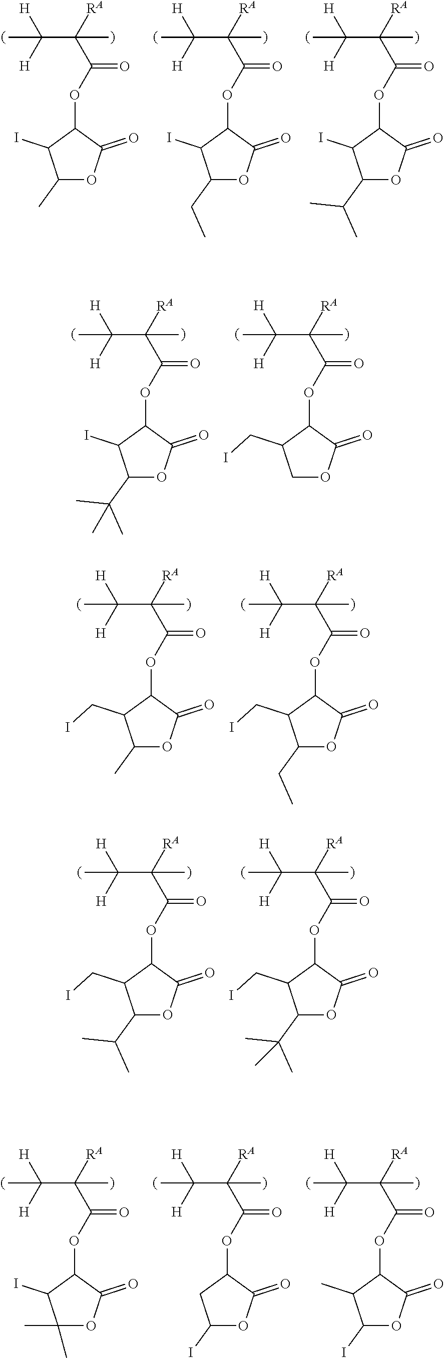

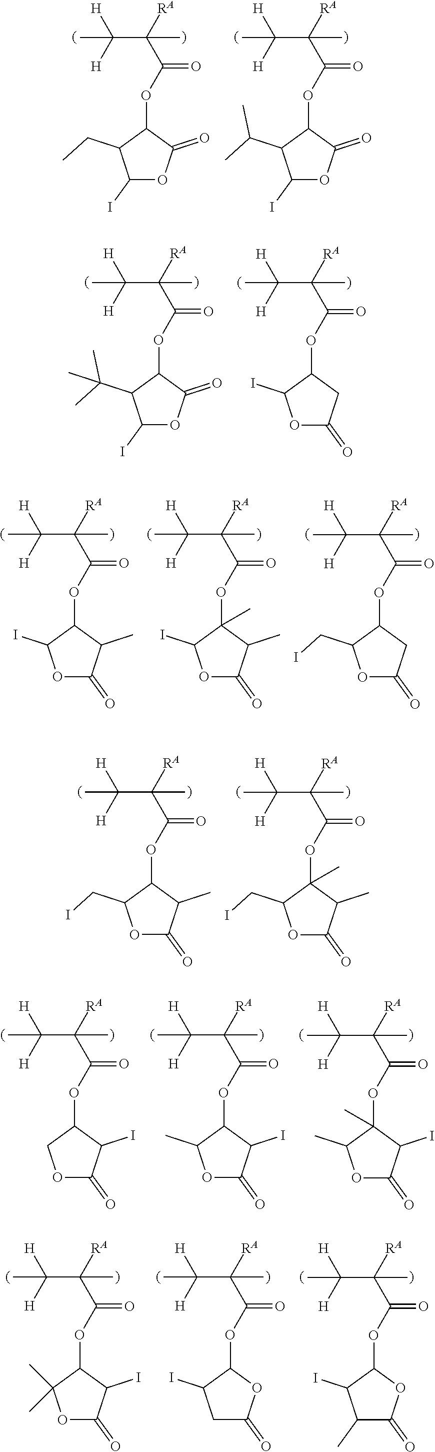

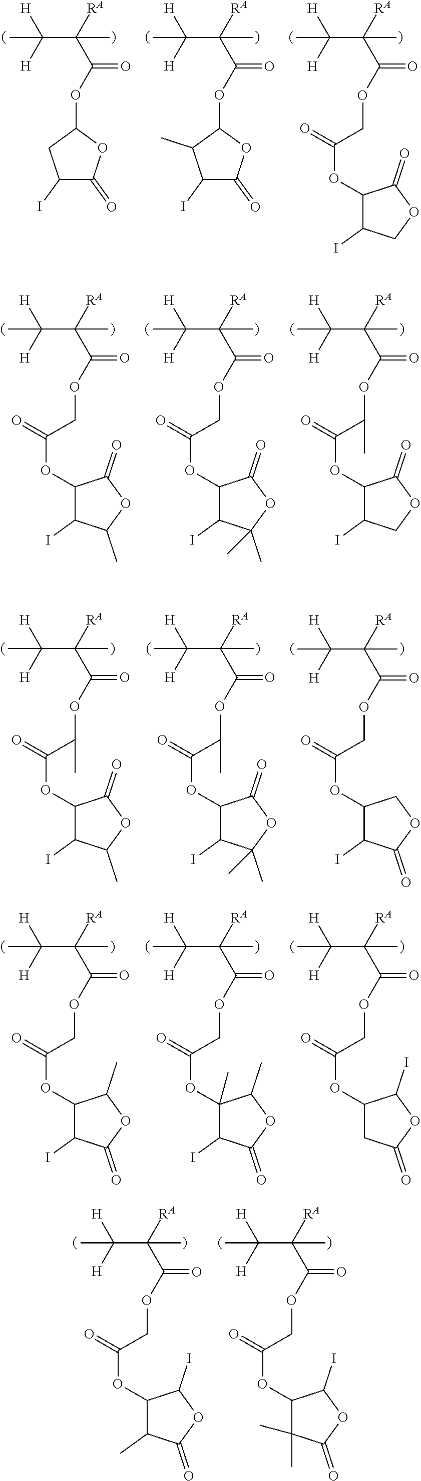

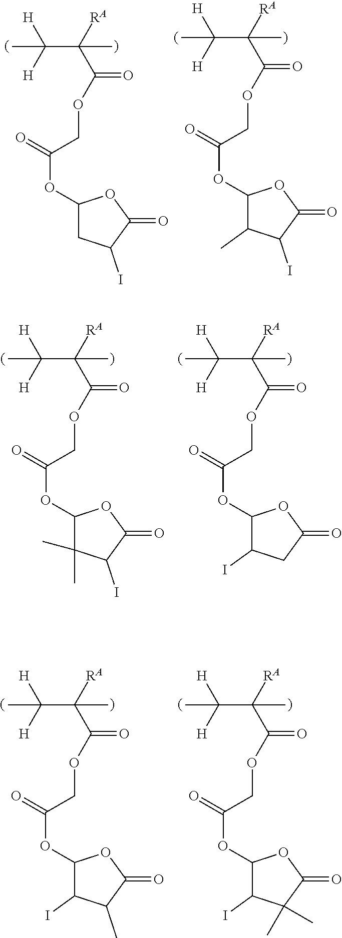

Specific examples of the repeating unit-b2 include, but are not limited to, the following. Note that in the following formulae, R^{A} is as defined above.

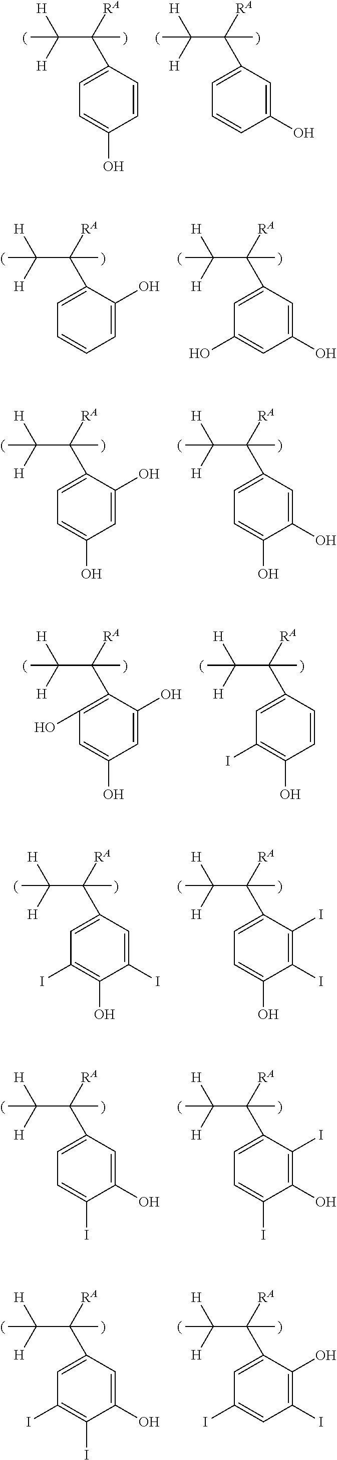

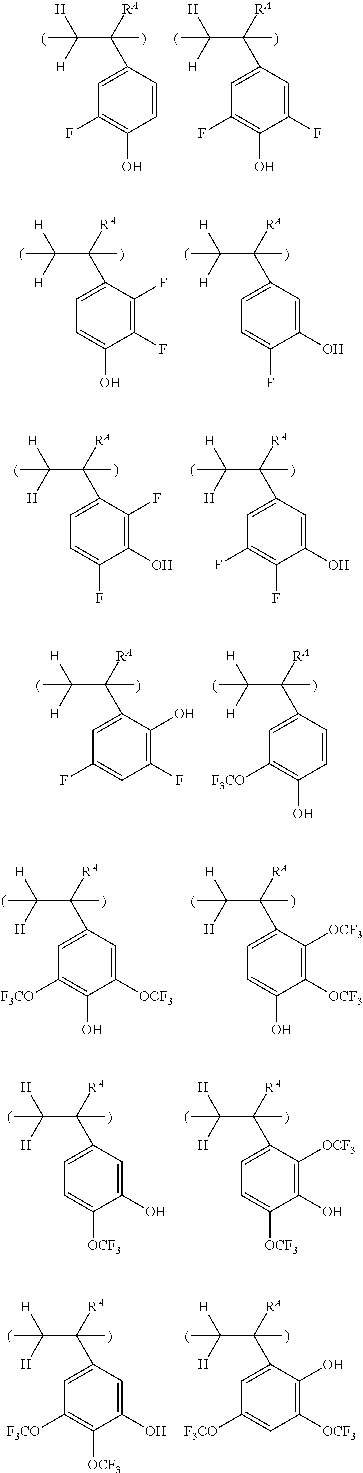

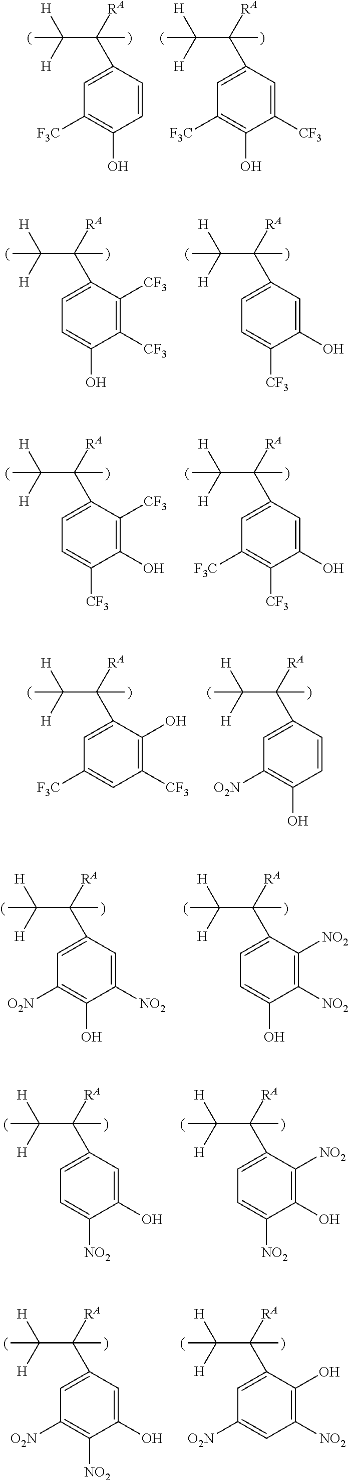

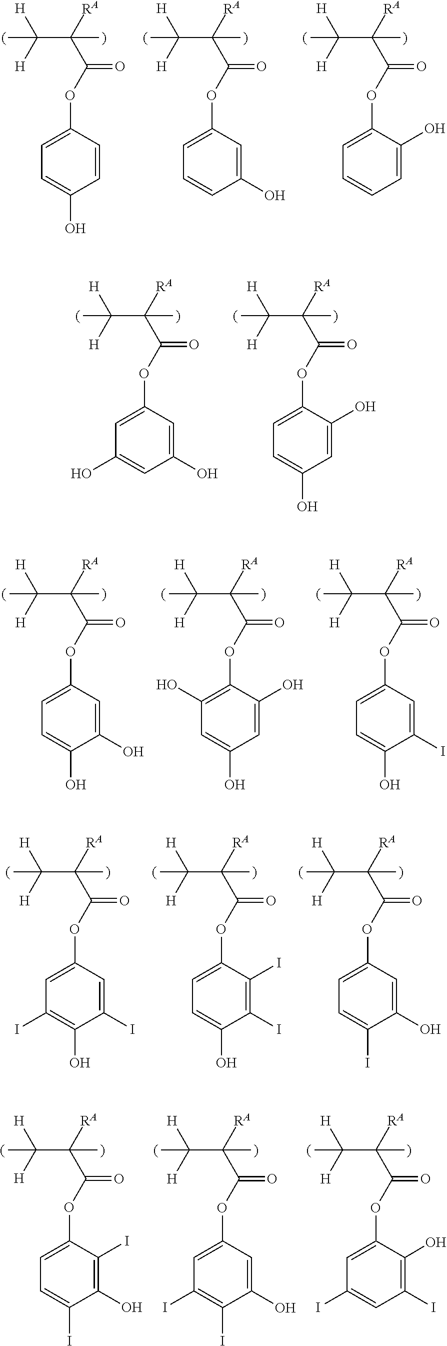

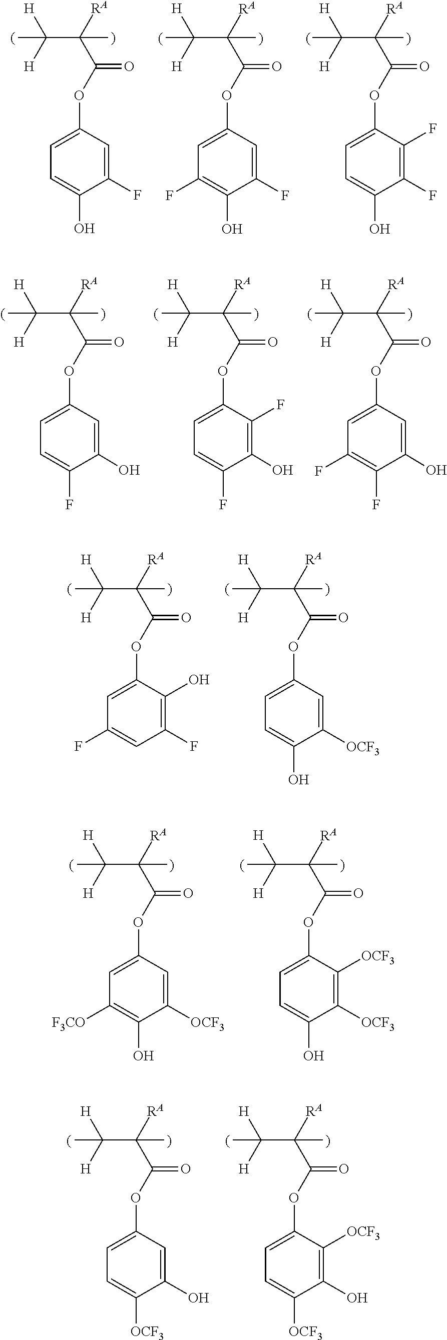

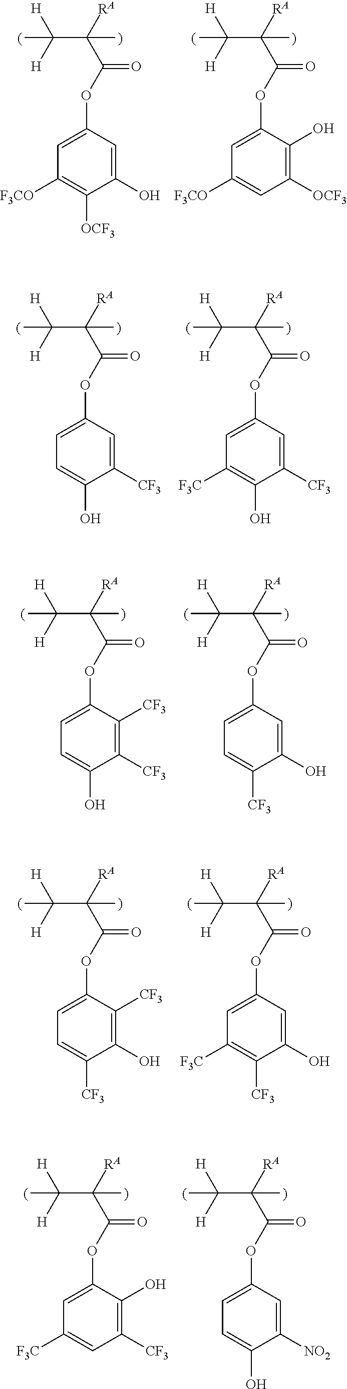

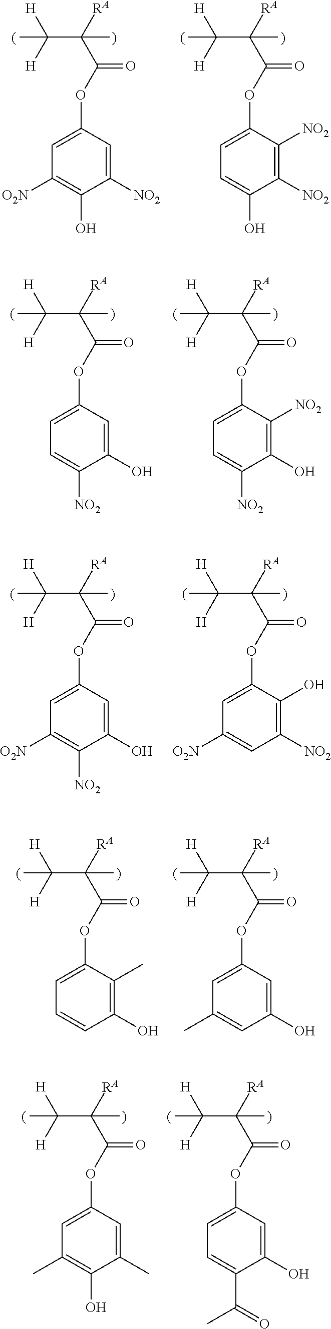

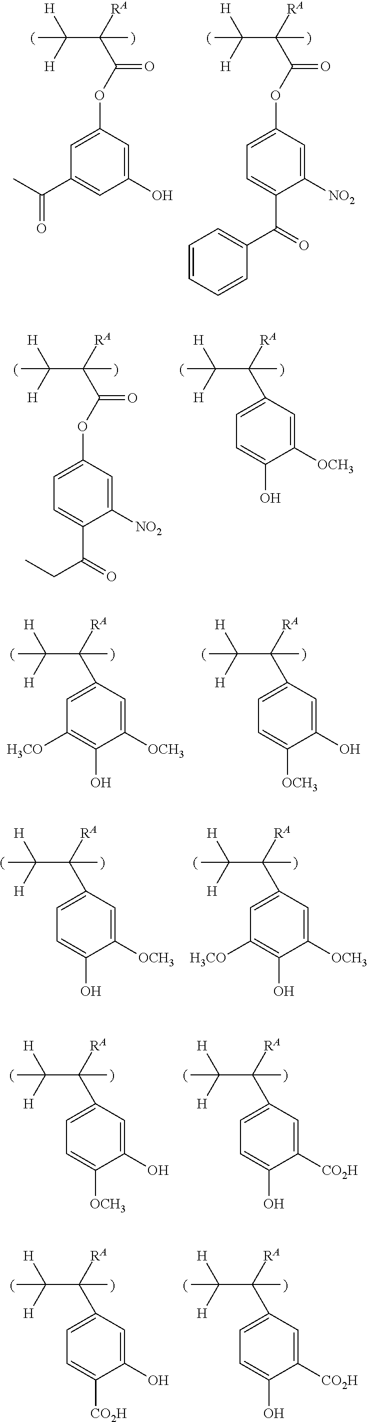

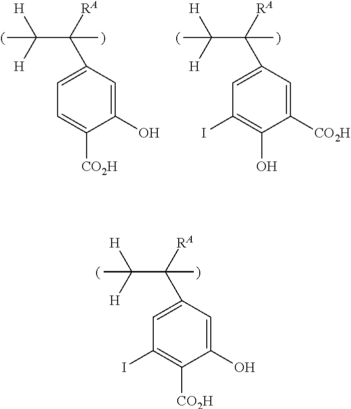

As the repeating unit-b1 or b2, in ArF lithography, it is particularly preferable that the repeating unit has a lactone ring as a polar group, and in KrF lithography, EB lithography, and EUV lithography, it is preferable that the repeating unit has a phenol moiety.

It is preferable that the base polymer further contains at least one repeating unit selected from the group consisting of a repeating unit represented by the following general formula (c1) (hereinafter also referred to as “repeating unit-c1”), a repeating unit represented by the following general formula (c2) (hereinafter also referred to as “repeating unit-c2”), a repeating unit represented by the following general formula (c3) (hereinafter also referred to as “repeating unit-c3”), a repeating unit represented by the following general formula (c4) (hereinafter also referred to as “repeating unit-c4”), and a repeating unit represented by the following general formula (c5) (hereinafter also referred to as “repeating unit-c5”).

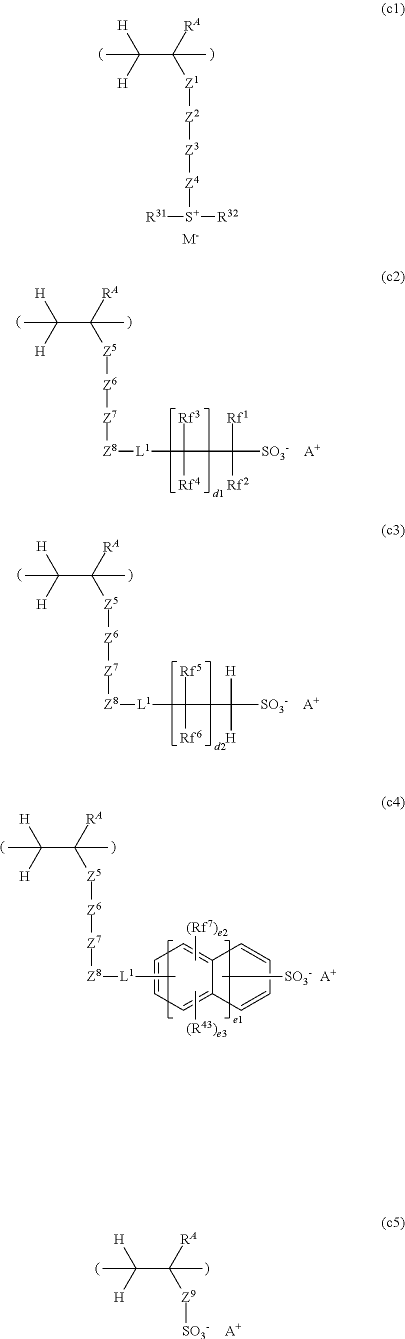

wherein “d1” and “d2” each independently represent an integer from 0 to 3;
“e1” represents 0 or 1; “e2” represents an integer from 0 to 4; “e3” represents an integer from 0 to 4; however, when “e1” represents 0, 0≤e2+e3≤4, and when “e1” represents 1, 0≤e2+e3≤6;
R^{A} each independently represents a hydrogen atom, a fluorine atom, a methyl group, or a trifluoromethyl group;
Z¹ represents a single bond or a phenylene group which may have a substituent;
Z² represents a single bond, **—C(═O)—O—Z²¹—, **—C(═O)—NH—Z²¹—, or **—O—Z²¹—; Z²1 represents an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ represents a single bond, an ether bond, an ester bond, a sulfonate ester bond, an amide bond, a sulfonamide bond, a carbonate bond, or a carbamate bond;
Z⁴ represents a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ each independently represents a single bond, a phenylene group or naphthylene group which may have a substituent, or *—C(═O)—O—Z⁵¹—; Z⁵¹ represents an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ represents a single bond, an ether bond, an ester bond, a sulfonate ester bond, an amide bond, a sulfonamide bond, a carbonate bond, or a carbamate bond;
Z⁷ each independently represents a single bond, ***—Z⁷¹—C(═O)—O—, ***—C(═O)—NH—Z⁷¹—, or ***—O—Z⁷¹—; Z⁷ represents a hydrocarbylene group having 1 to 20 carbon atoms and optionally containing a heteroatom;
Z⁸ each independently represents a single bond, ****—Z⁸¹—C(═O)—O—, ****—C(═O)—NH—Z⁸¹—, or ****—O—Z⁸¹—; Z⁸¹ represents a hydrocarbylene group having 1 to 20 carbon atoms and optionally containing a heteroatom;
Z⁹ represents a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—; Z⁹¹ represents an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
* represents an attachment point to a main chain carbon atom; “**” represents an attachment point to Z¹; “***” represents an attachment point to Z⁶; “****” represents an attachment point to Z⁷;
L¹ represents a single bond, an ether bond, an ester bond, a carbonyl group, a sulfonate ester bond, a sulfonamide bond, a carbonate bond, or a carbamate bond;
Rf¹ and Rf² each independently represent a fluorine atom or a fluorinated saturated hydrocarbyl group having 1 to 6 carbon atoms;
Rf³ and Rf⁴ each independently represent a hydrogen atom, a fluorine atom, or a fluorinated saturated hydrocarbyl group having 1 to 6 carbon atoms;
Rf⁵ and Rf⁶ each independently represent a hydrogen atom, a fluorine atom, or a fluorinated saturated hydrocarbyl group having 1 to 6 carbon atoms; however, all Rf⁵ and Rf⁶ cannot simultaneously become a hydrogen atom;
Rf⁷ represents a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² each independently represent a hydrocarbyl group having 1 to 20 carbon atoms and optionally containing a heteroatom; furthermore, R⁴¹ and R⁴² may be bonded to each other to form a ring together with the sulfur atom to which they are bonded;
R⁴³ represents a halogen atom other than a fluorine atom, or a hydrocarbyl group having 1 to 20 carbon atoms and optionally containing a heteroatom; when “e3” is 2, 3, or 4, a plurality of R⁴³ may be bonded to each other to form a ring together with the carbon atoms to which they are bonded;
M⁻ represents a non-nucleophilic counterion; and
A⁺ represents an onium cation.

In the general formulae (c1) to (c5), R^{A} each independently represents a hydrogen atom, a fluorine atom, a methyl group, or a trifluoromethyl group. Z¹ represents a single bond, or a phenylene group which may have a substituent. Z² represents a single bond, **—C(═O)—O—Z²¹—, **—C(═O)—NH—Z²¹—, or **—O—Z²¹—. Z²¹ represents an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group. Z³ represents a single bond, an ether bond, an ester bond, a sulfonate ester bond, an amide bond, a sulfonamide bond, a carbonate bond, or a carbamate bond. Z⁴ represents a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group. Z⁵ each independently represents a single bond, a phenylene group or a naphthylene group which may have a substituent, or *—C(═O)—O—Z⁵¹—. Z⁵¹ represents an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring. Z⁶ represents a single bond, an ether bond, an ester bond, a sulfonate ester bond, an amide bond, a sulfonamide bond, a carbonate bond, or a carbamate bond. Z⁷ each independently represents a single bond, ***—Z⁷¹—C(═O)—O—, ***—C(═O)—NH—Z⁷¹—, or ***—O—Z⁷¹—. Z⁷?represents a hydrocarbylene group having 1 to 20 carbon atoms and optionally containing a heteroatom. Z⁸ each independently represents a single bond, ****—Z—C(═O)—O—, ****—C(═O)—NH—Z⁸¹—, or ****—O—Z¹—. Z⁸¹ represents a hydrocarbylene group having 1 to 20 carbon atoms and optionally containing a heteroatom. Z⁹ represents a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—. Z⁹¹ represents an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group. * represents an attachment point to a main chain carbon atom. ** represents an attachment point to Z¹. *** represents an attachment point to Z⁶. **** represents an attachment point to Z⁷.

The aliphatic hydrocarbylene group represented by Z²¹, Z⁵¹, and Z⁹¹ may be linear, branched, or cyclic, and specific examples thereof include alkanediyl groups of methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl groups; cycloalkanediyl groups such as cyclopropanediyl, cyclobutanediyl, cyclopentanediyl, and cyclohexanediyl groups; and groups obtained by combining these, etc.

The hydrocarbylene group optionally containing a heteroatom represented by Z⁷¹ and Z⁸¹ may be saturated or unsaturated, and may be linear, branched, or cyclic. Specific examples thereof include, but are not limited to, the following.

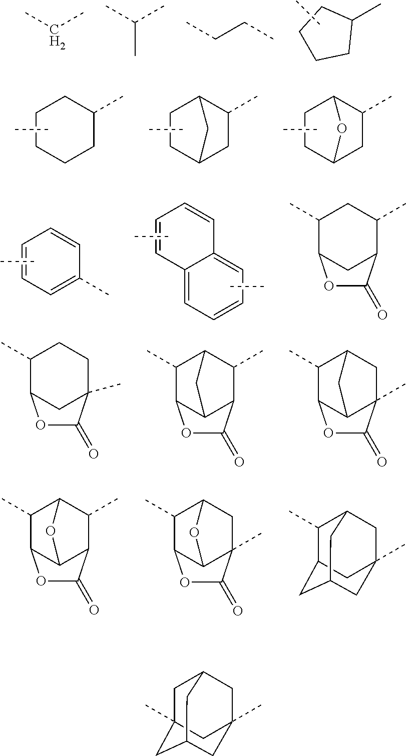

In the formulae, a broken line represents an attachment point.

In the general formula (c1), R⁴¹ and R⁴² each independently represent a hydrocarbyl group having 1 to 20 carbon atoms and optionally containing a heteroatom. The hydrocarbyl group may be saturated or unsaturated and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, 1-propenyl, 2-propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl, naphthyl, and thienyl groups; aralkyl groups having 7 to 20 carbon atoms, such as benzyl, 1-phenylethyl, and 2-phenylethyl groups; and groups obtained by combining these, etc.; however, an aryl group is preferable. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Furthermore, R⁴¹ and R⁴² may be bonded to each other to form a ring together with the sulfur atom to which they are bonded. In this case, specific examples of the ring include those represented by the following formulae, etc.

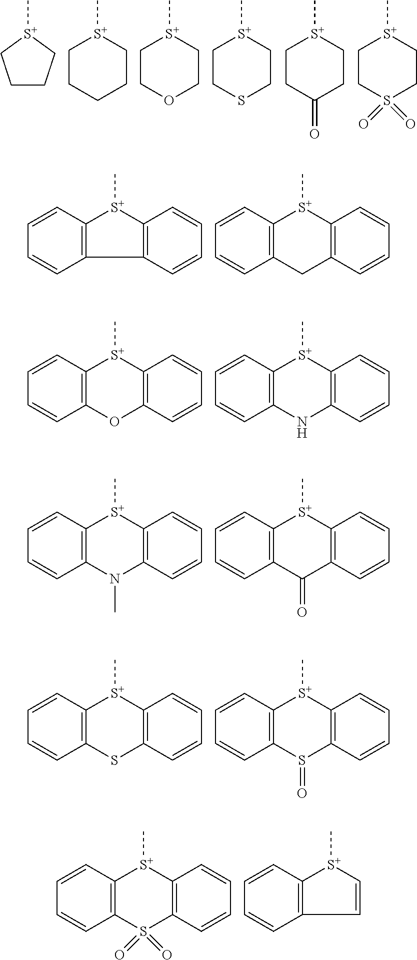

In the formulae, a broken line represents an attachment point to Z⁴.

Specific examples of the cation of the repeating unit-c1 include, but are not limited to, the following. Note that in the following formulae, R^{A} is as defined above.

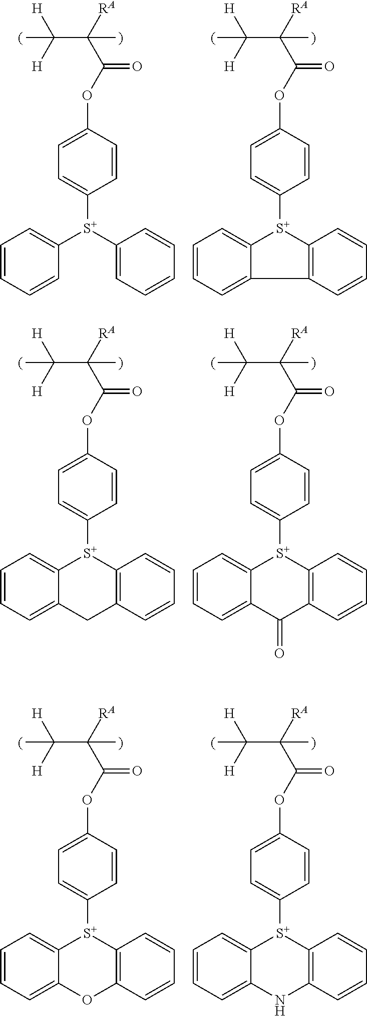

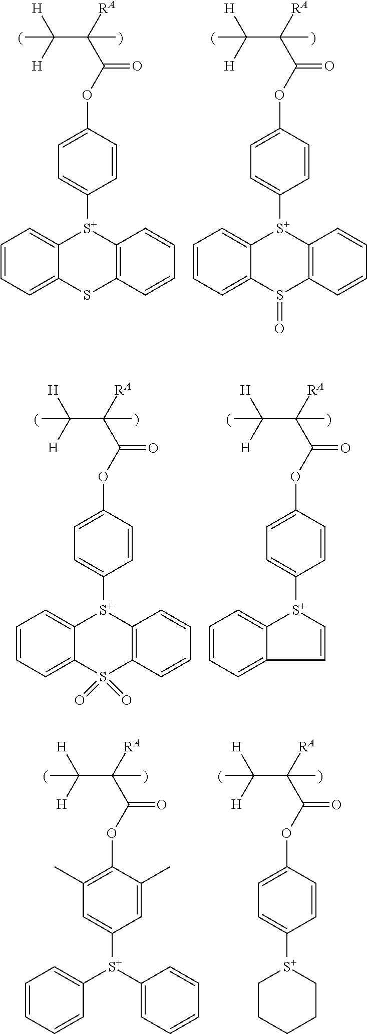

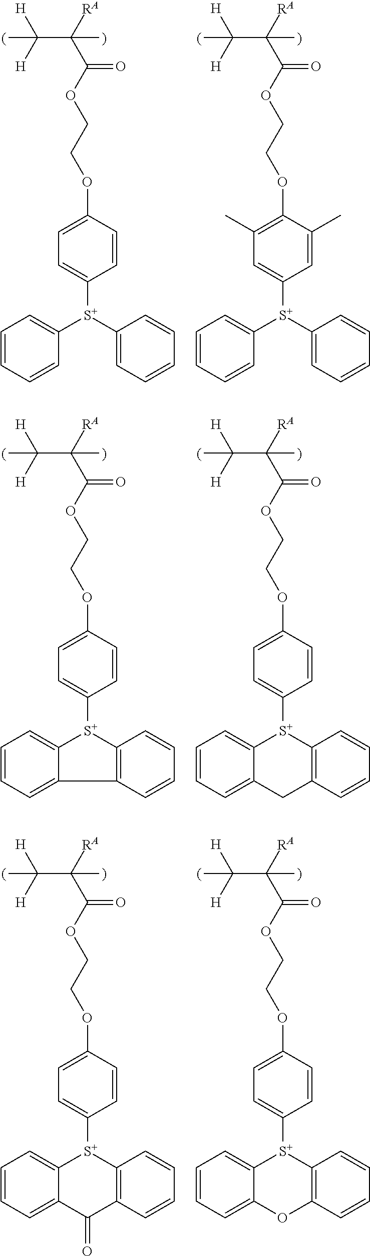

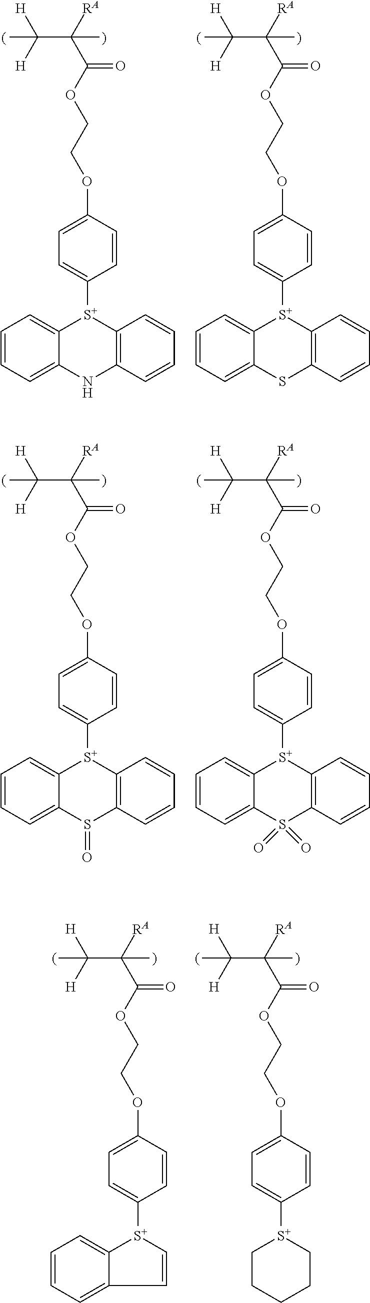

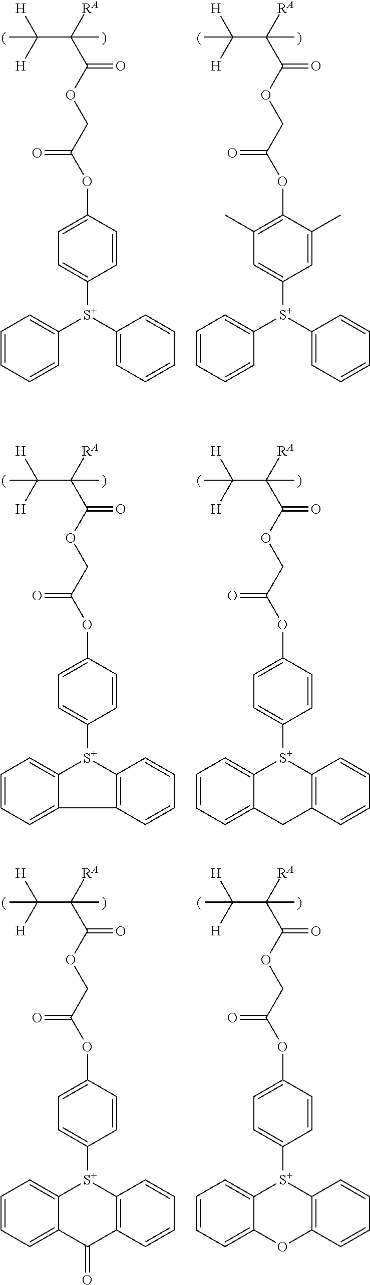

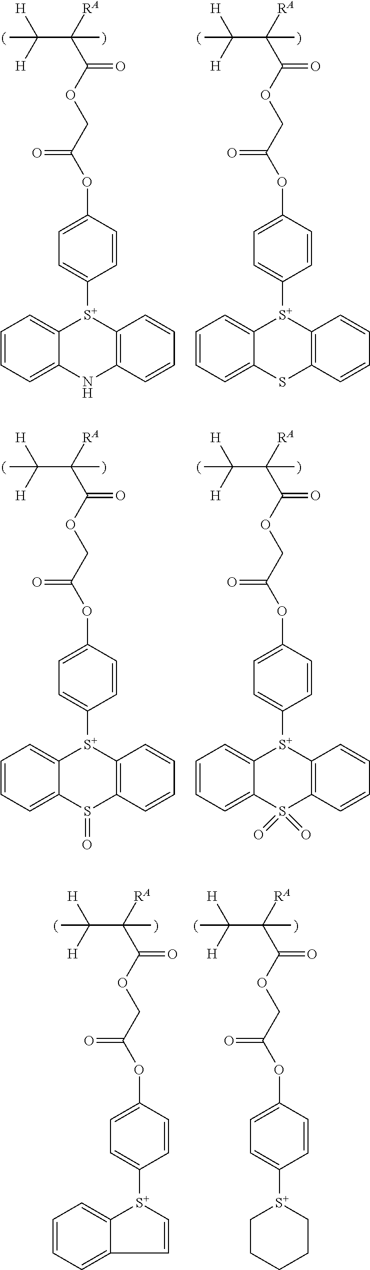

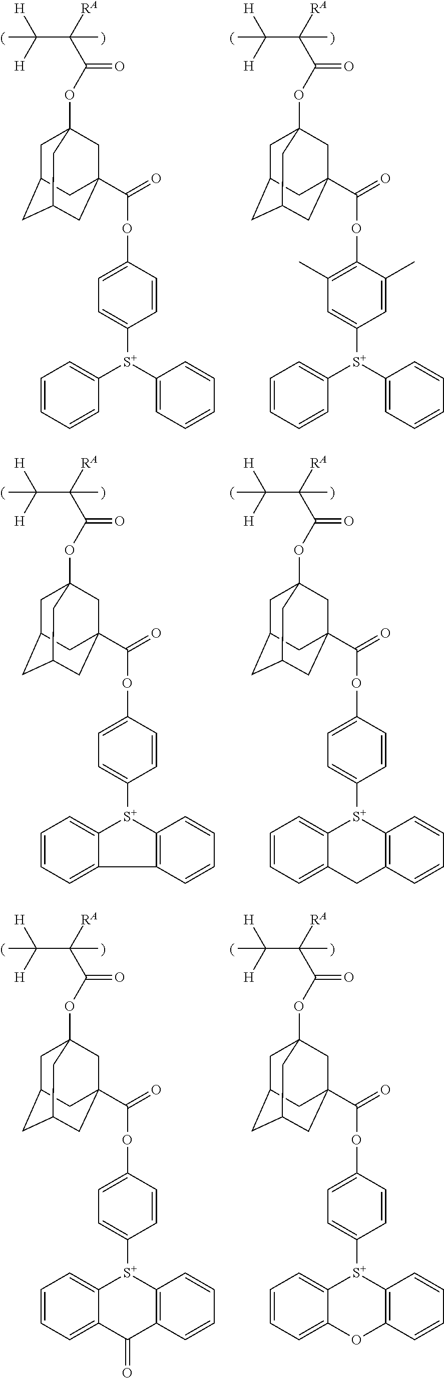

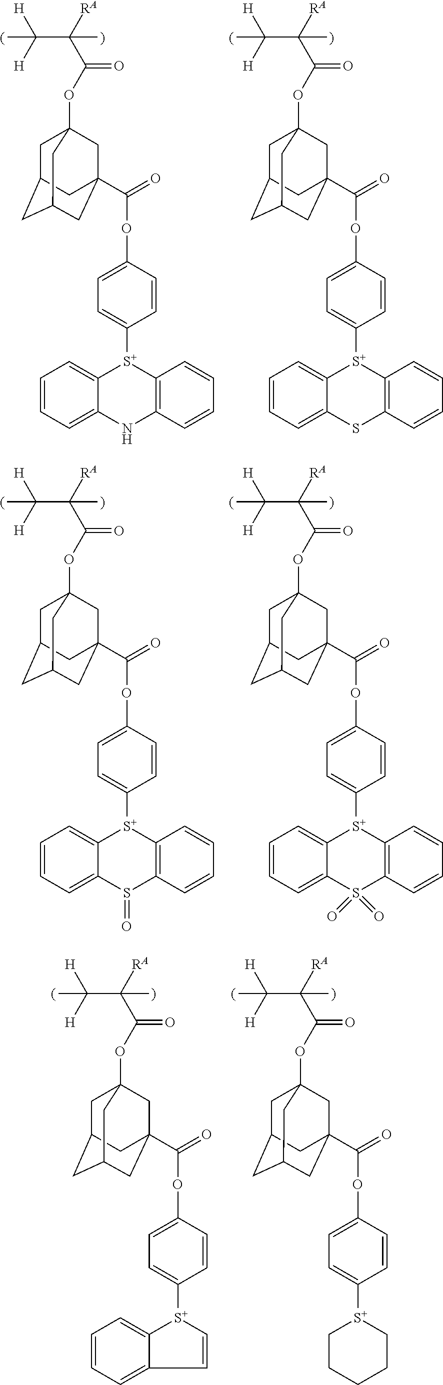

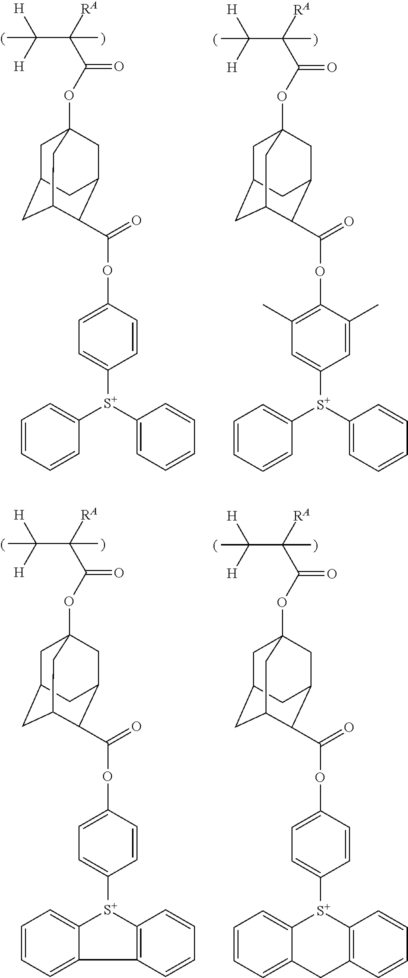

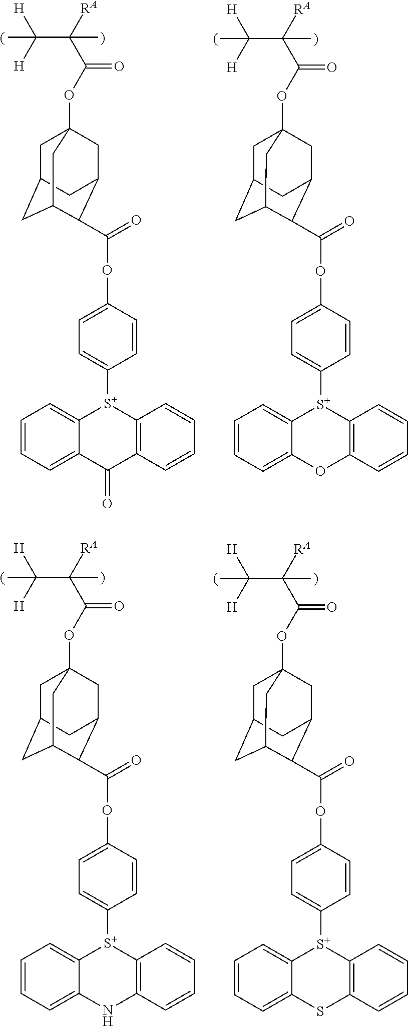

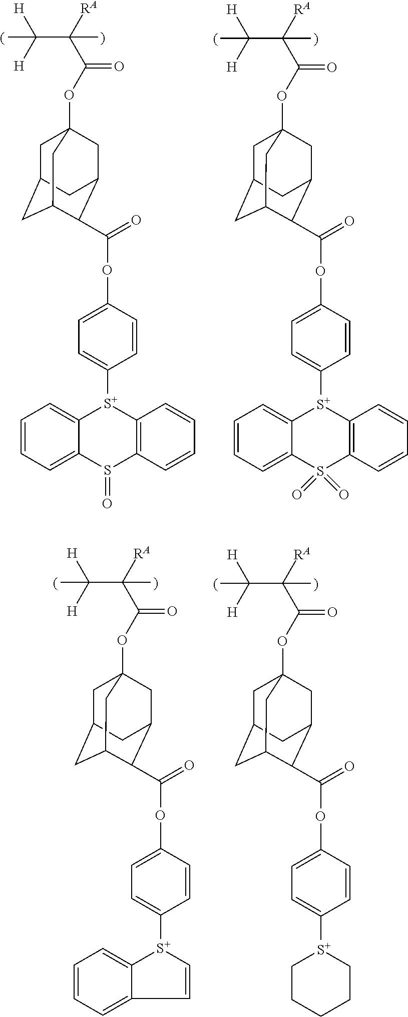

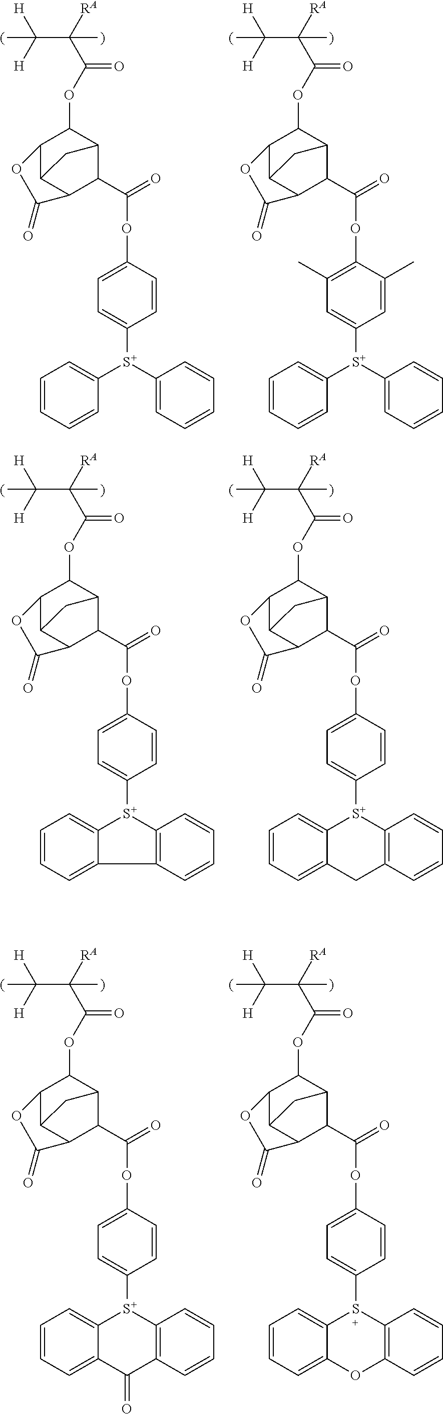

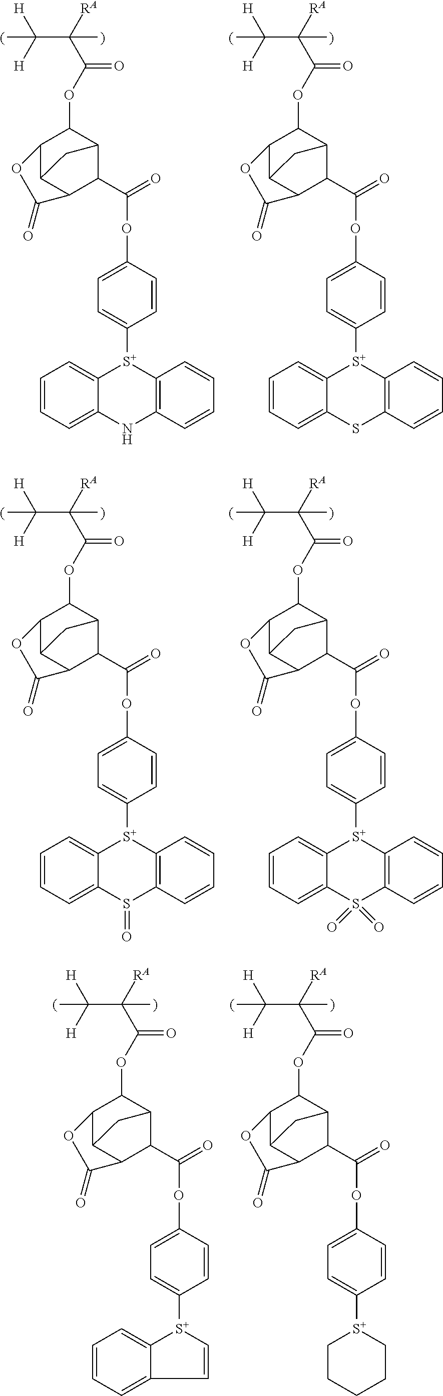

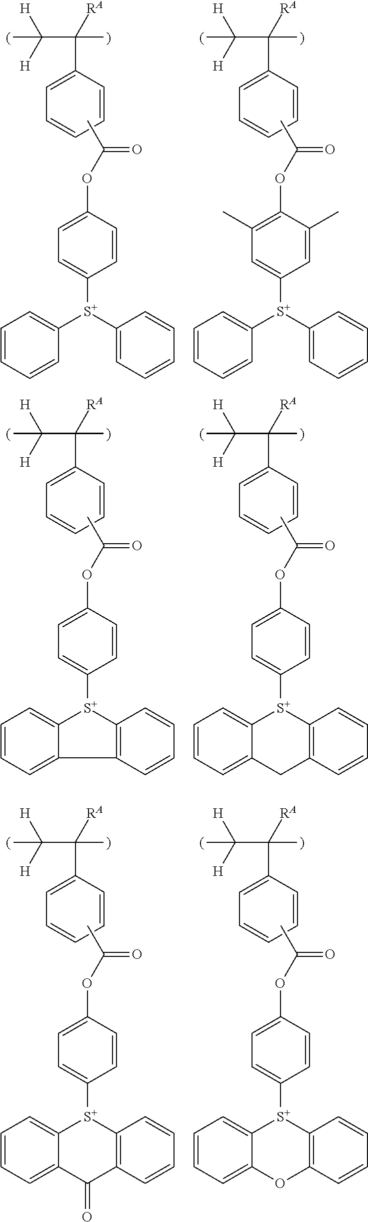

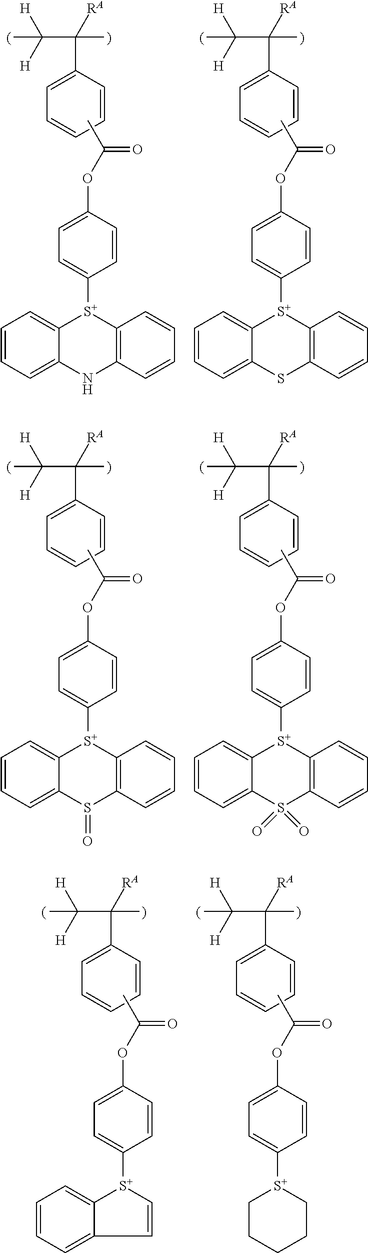

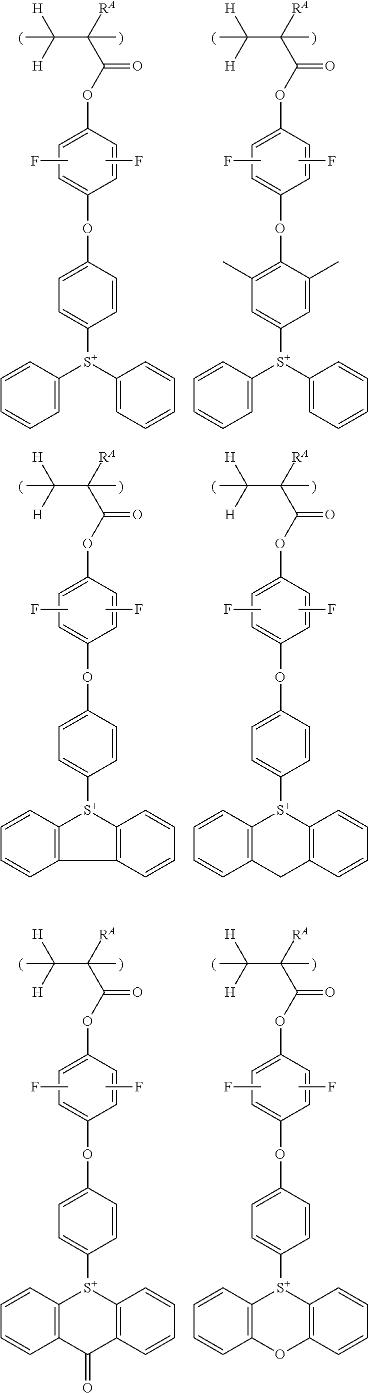

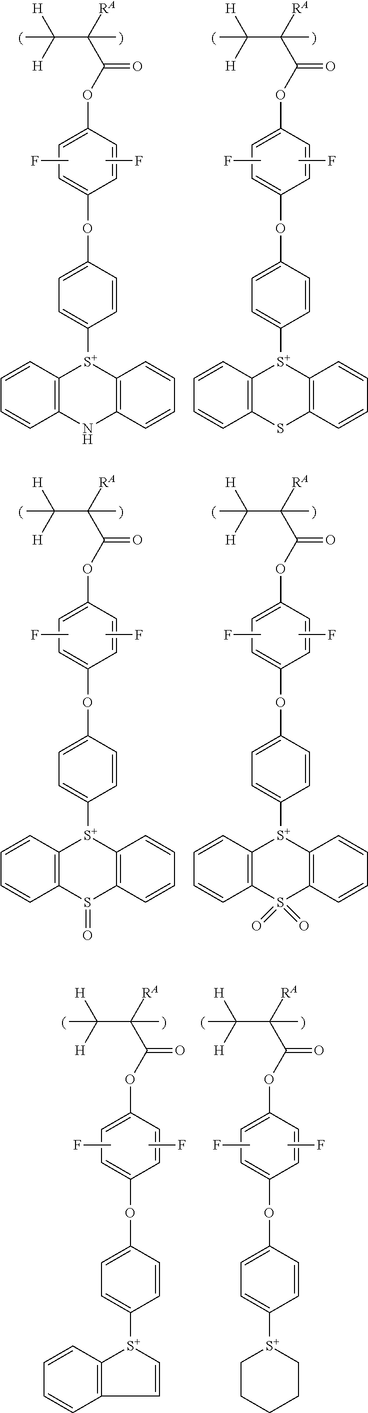

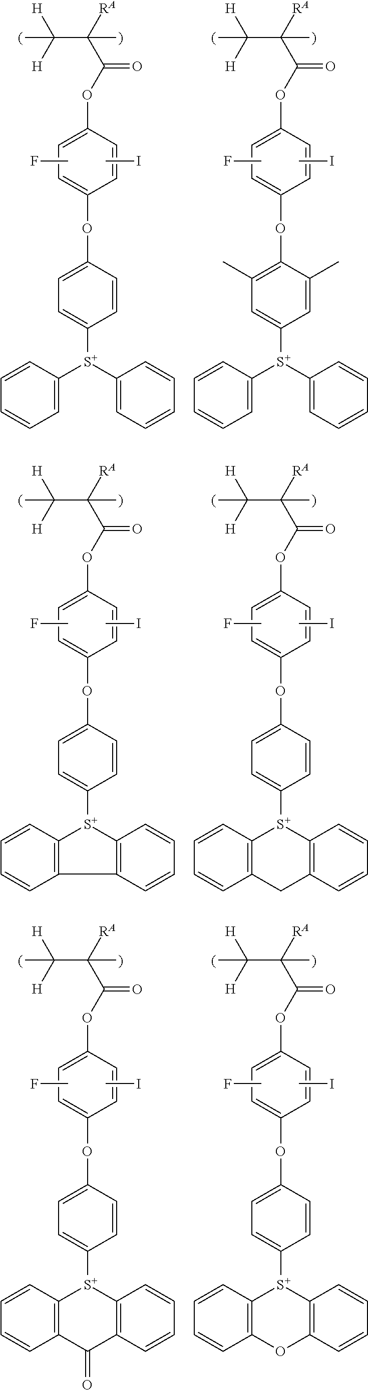

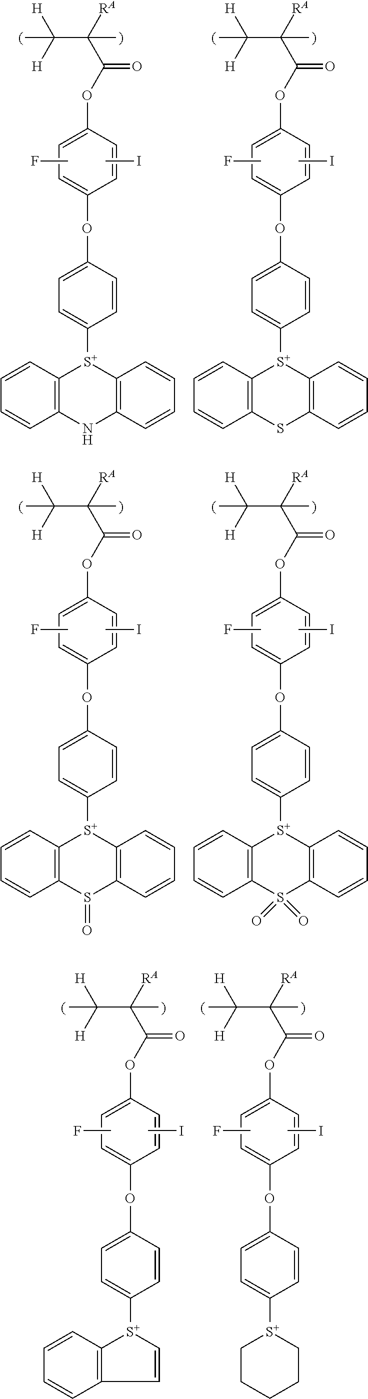

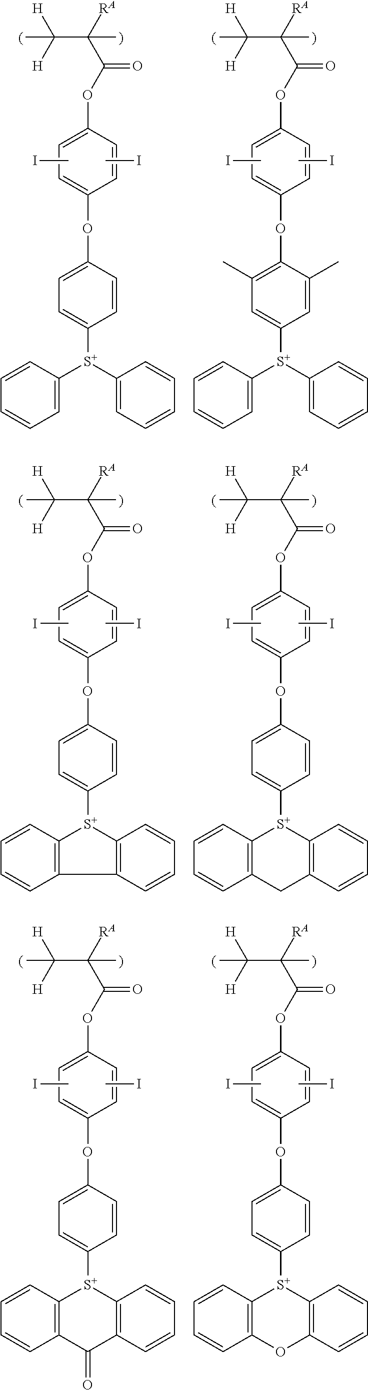

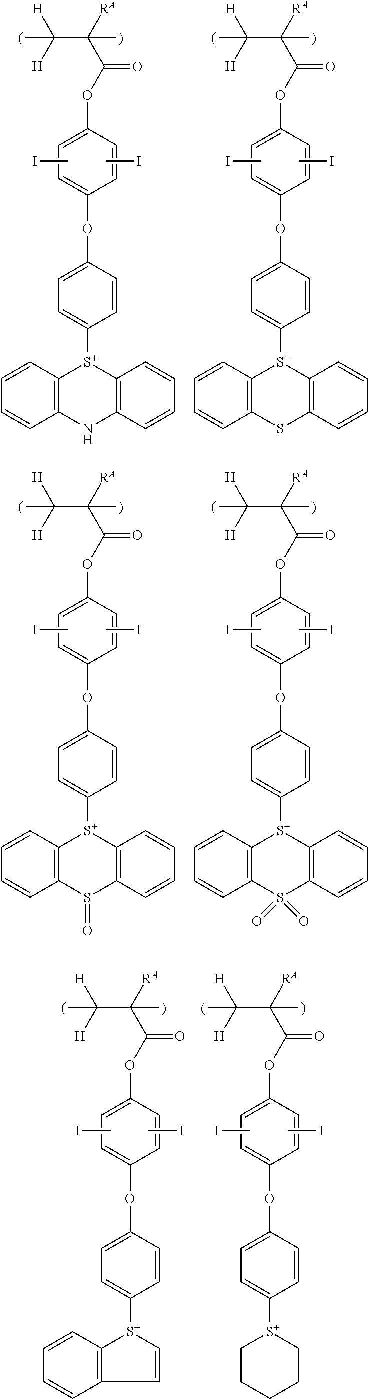

In the general formula (c1), M⁻ represents a non-nucleophilic counterion. The non-nucleophilic counterion is preferably a halide ion, a sulfonate anion, an imidic acid anion, or a methide acid anion. Specific examples of the halide ion include a chloride ion, a bromide ion, etc. Specific examples of the sulfonate anion (sulfonate ion) include fluoroalkylsulfonate ions such as a triflate ion, a 1,1,1-trifluoroethanesulfonate ion, and a nonafluorobutanesulfonate ion; arylsulfonate ions such as a tosylate ion, a benzenesulfonate ion, a 4-fluorobenzenesulfonate ion, and a 1,2,3,4,5-pentafluorobenzenesulfonate ion; and alkylsulfonate ions such as a mesylate ion and a butanesulfonate ion. Specific examples of the imide acid anion (imide ion) include a bis(trifluoromethylsulfonyl)imide ion, a bis(perfluoroethylsulfonyl)imide ion, a bis(perfluorobutylsulfonyl)imide ion, etc. Specific examples of the methide acid anion (methide ion) include a tris(trifluoromethylsulfonyl)methide ion, a tris(perfluoroethylsulfonyl)methide ion, etc.

Other examples of the non-nucleophilic counterion include anions represented by any of the following general formulae (c1-1) to (c1-4).

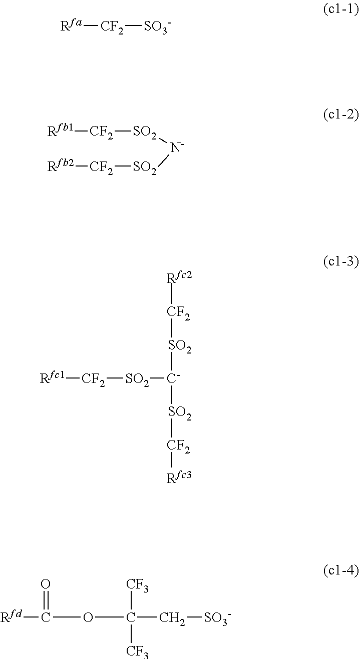

In the general formula (c1-1), R^{fa} represents a fluorine atom, or a hydrocarbyl group having 1 to 40 carbon atoms and optionally containing a heteroatom. The hydrocarbyl group may be saturated or unsaturated and may be linear, branched, or cyclic. Specific examples thereof include the same ones as those exemplified as the hydrocarbyl group represented by R^{fa1} in the general formula (c1-1-1) described below.

The anion represented by the general formula (c1-1) is preferably represented by the following general formula (c1-1-1).

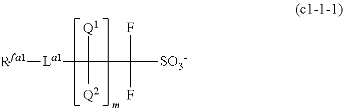

In the general formula (c1-1-1), Q¹ and Q² each independently represent a hydrogen atom, a fluorine atom, or a fluorinated saturated hydrocarbyl group having 1 to 6 carbon atoms, but in order to improve solvent solubility, it is preferable that at least one of them is a trifluoromethyl group. “m” represents 0, 1, 2, 3 or 4, with 1 being particularly preferable. R^{fa1} represents a hydrocarbyl group having 1 to 35 carbon atoms and optionally containing a heteroatom. The heteroatom is preferably an oxygen atom, a nitrogen atom, a sulfur atom, a halogen atom, etc., and more preferably an oxygen atom. The hydrocarbyl group particularly preferably has 6 to 30 carbon atoms, from the viewpoint of obtaining high resolution in fine pattern formation.

In the general formula (c1-1-1), the hydrocarbyl group having 1 to 35 carbon atoms represented by R^{fa1} may be saturated or unsaturated, and may be linear, branched, or cyclic. Specific examples thereof include alkyl groups having 1 to 35 carbon atoms, such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl groups; cyclic saturated hydrocarbyl groups having 3 to 35 carbon atoms, such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, dicyclohexylmethyl groups; unsaturated aliphatic hydrocarbyl groups having 2 to 35 carbon atoms, such as 2-propenyl and 3-cyclohexenyl groups; aryl groups having 6 to 35 carbon atoms, such as phenyl, 1-naphthyl, 2-naphthyl, 9-fluorenyl groups; aralkyl groups having 7 to 35 carbon atoms, such as benzyl and diphenylmethyl groups; and groups obtained by combining these, etc.

In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. Specific examples of hydrocarbyl groups containing heteroatoms include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups, etc.

In the general formula (c1-1-1), L^{a1} represents a single bond, an ether bond, an ester bond, a sulfonate ester bond, a carbonate bond, or a carbamate bond, but from the viewpoint of synthesis, it is preferably an ether bond or an ester bond, and more preferably an ester bond.

Specific examples of the anion represented by the general formula (c1-1) include, but are not limited to, the following. Note that in the following formulae, Q¹ is as defined above, and Ac represents an acetyl group.

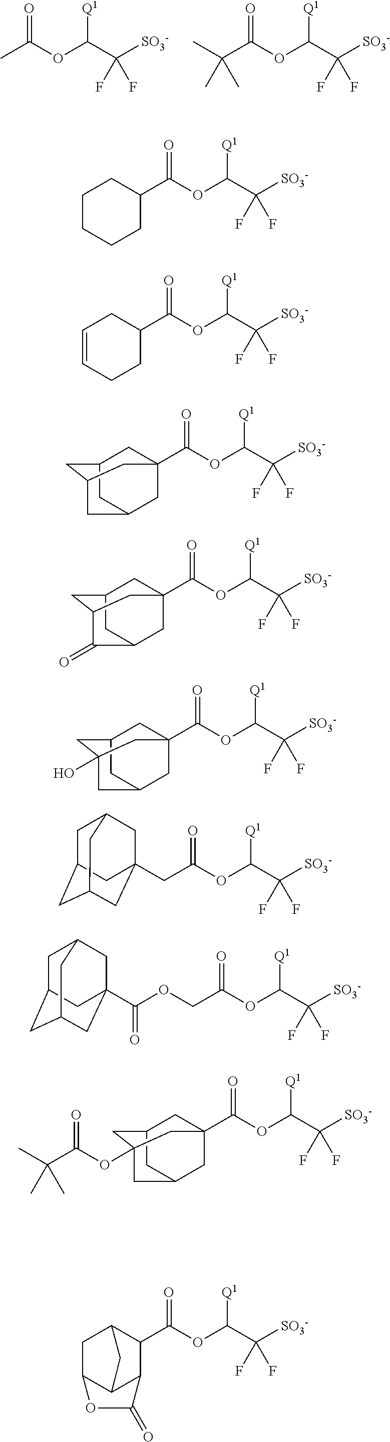

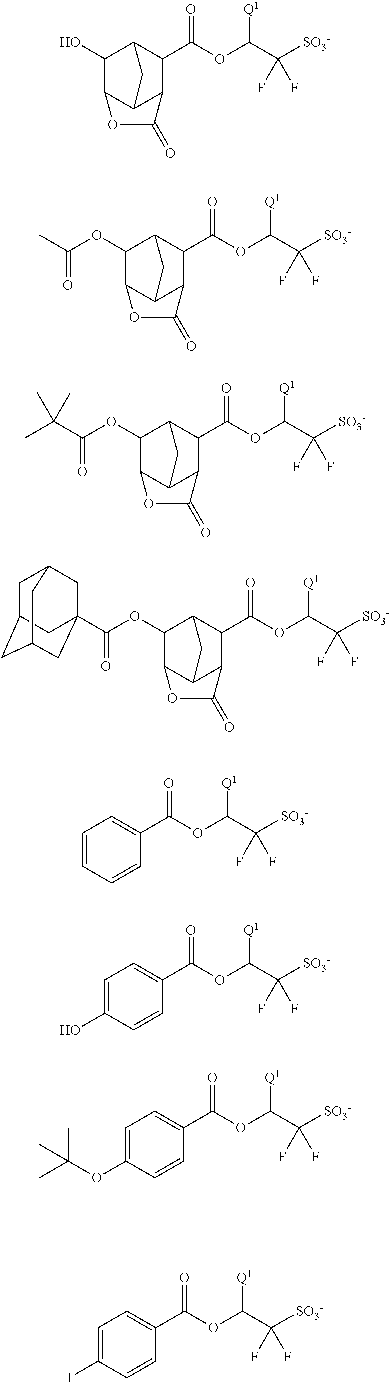

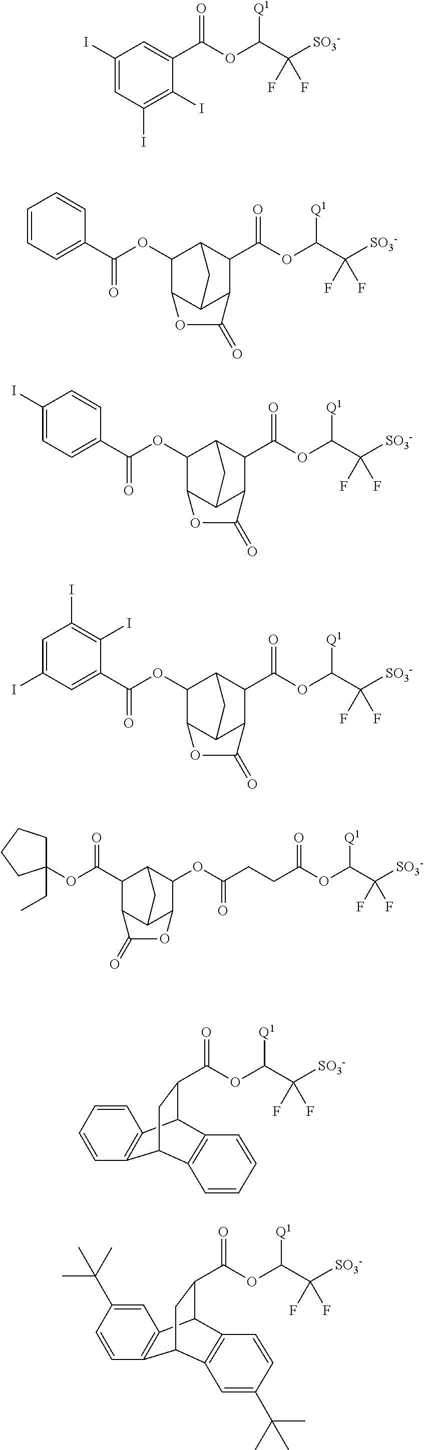

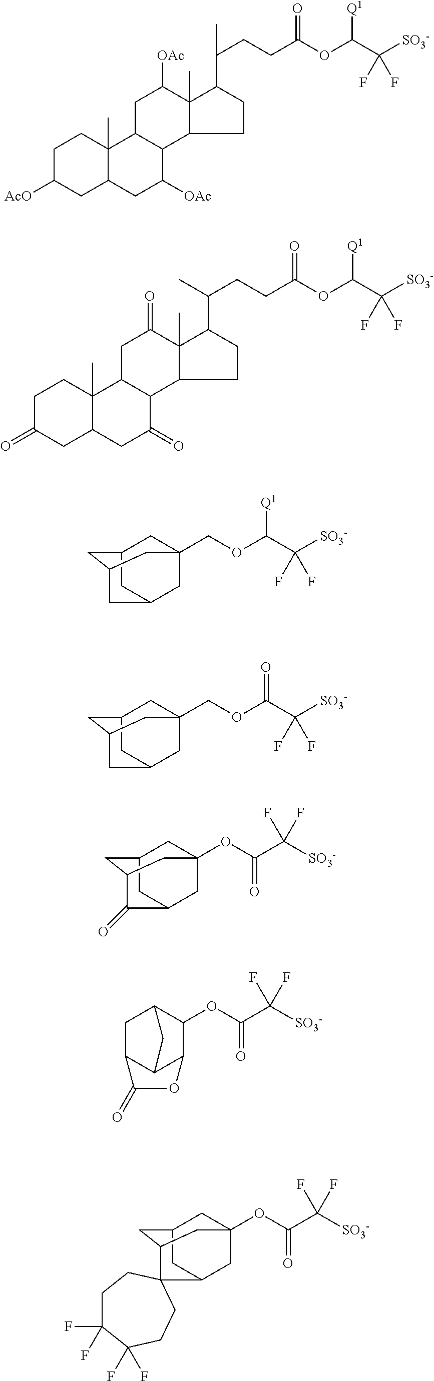

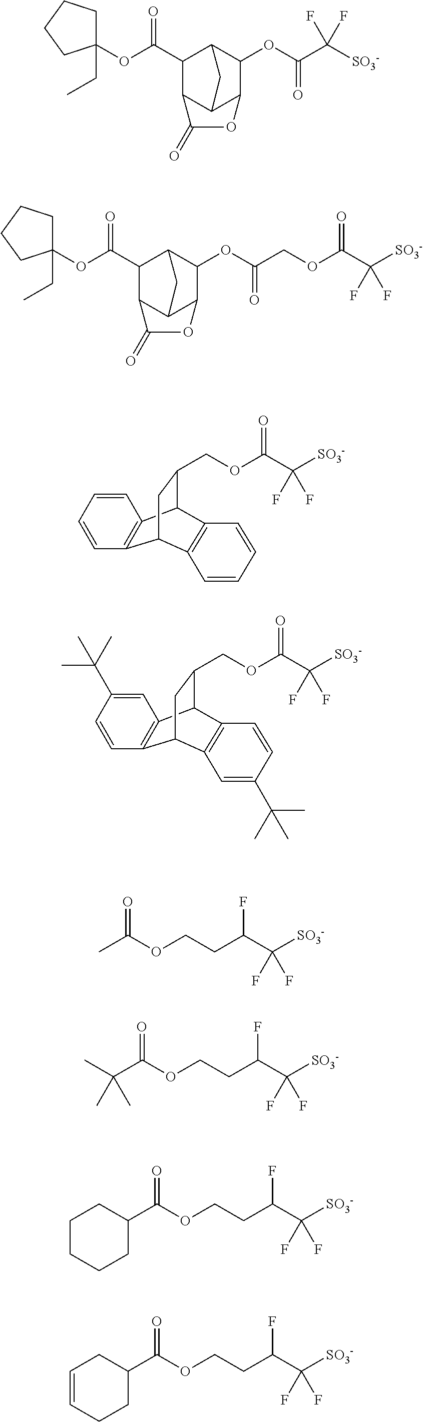

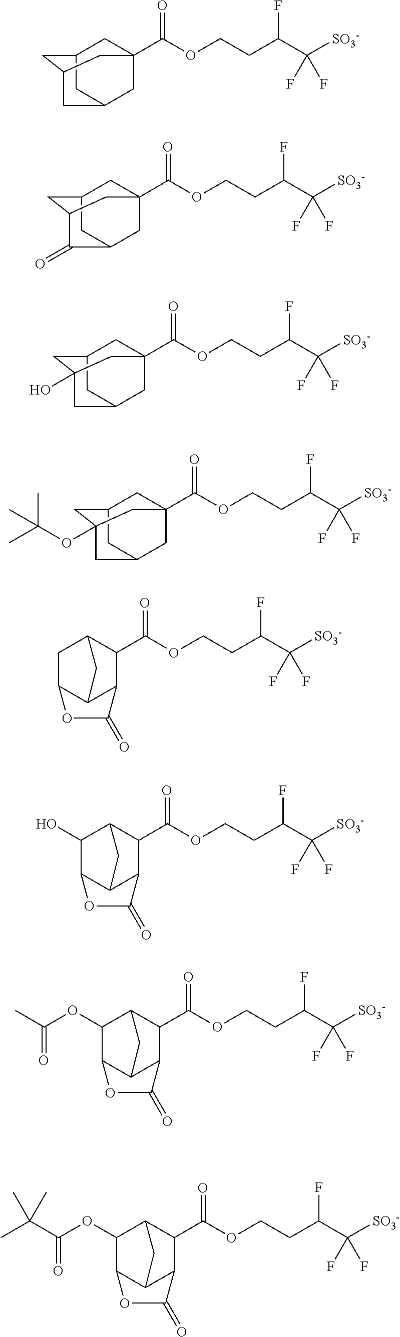

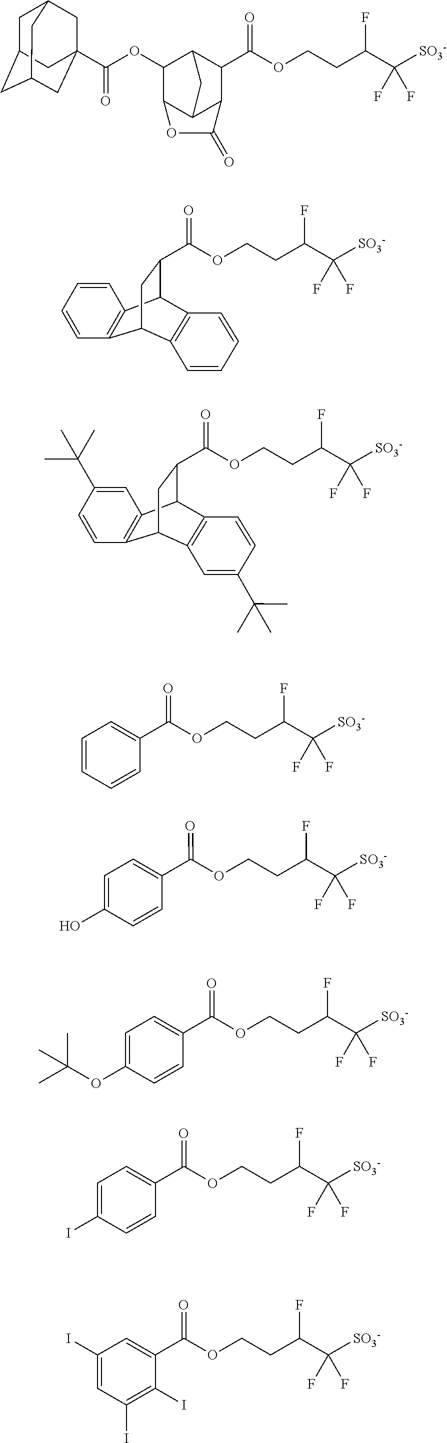

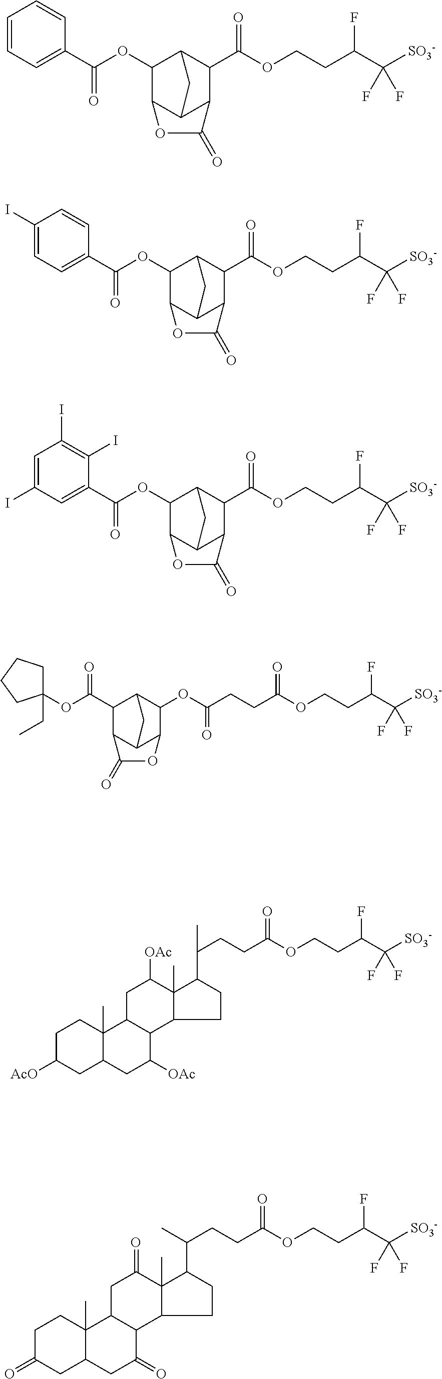

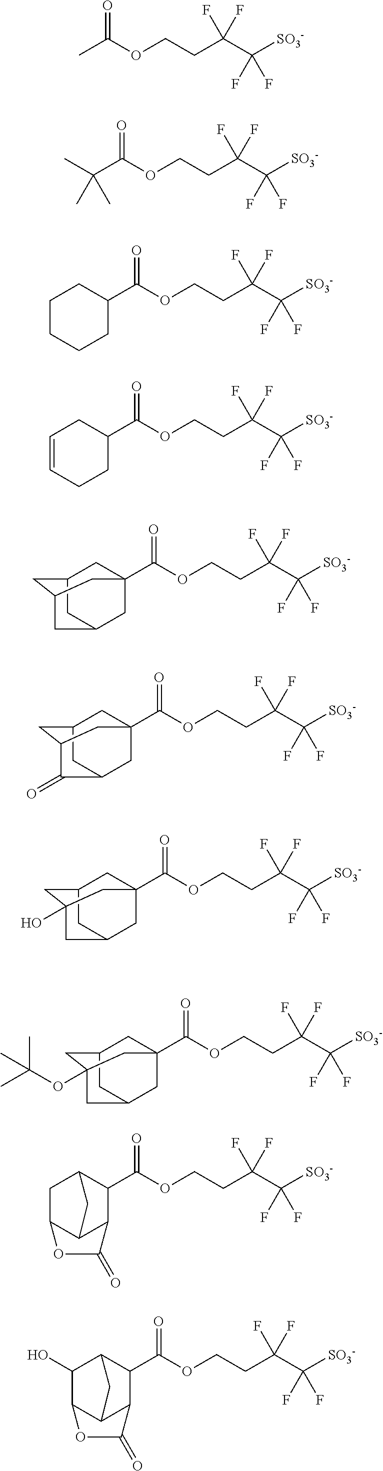

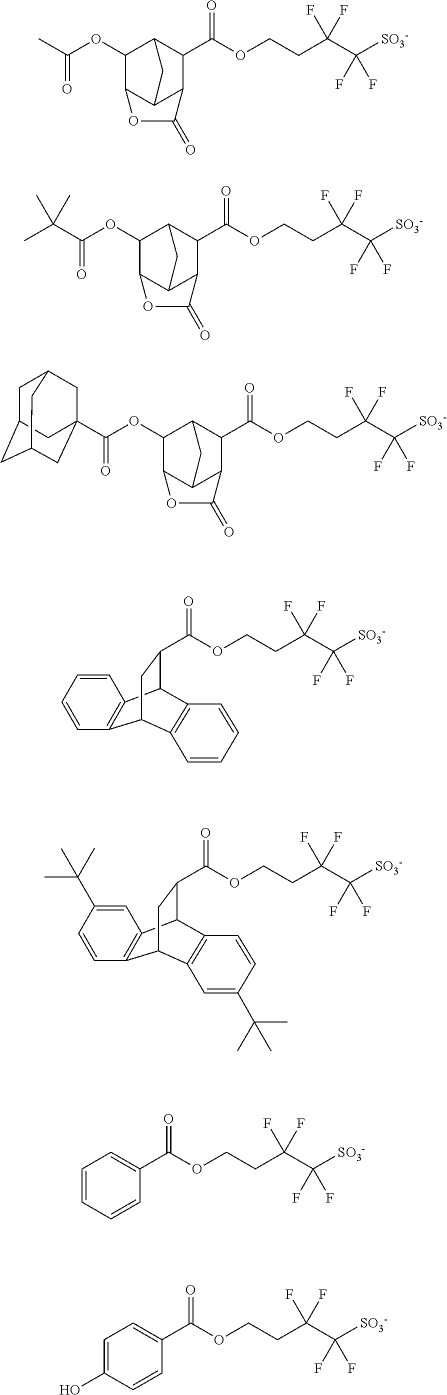

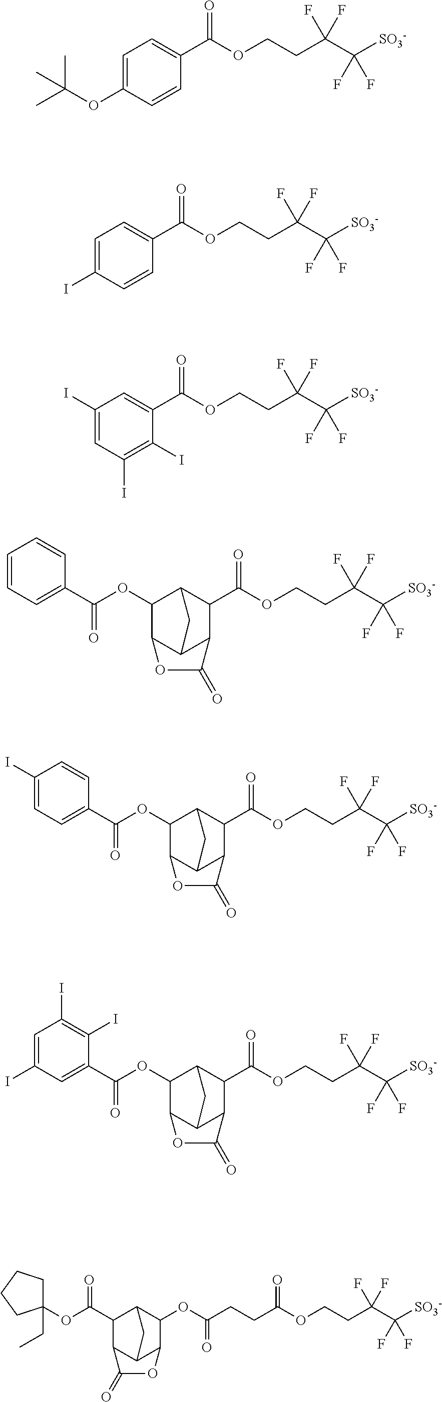

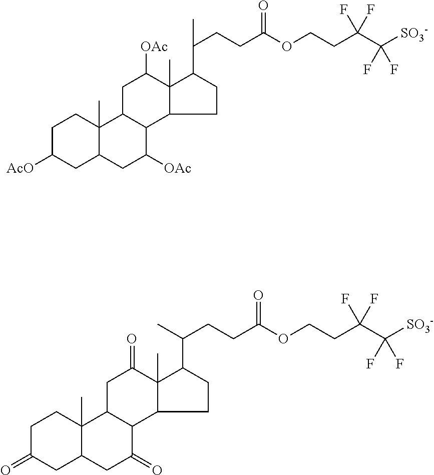

In the general formula (c1-2), R^{fb1} and R^{fb2} each independently represent a fluorine atom, or a hydrocarbyl group having 1 to 40 carbon atoms and optionally containing a heteroatom. The hydrocarbyl group may be saturated or unsaturated and may be linear, branched, or cyclic. Specific examples thereof include the same ones as those exemplified as the hydrocarbyl group represented by Ral in the general formula (c1-1-1). R^{fb1} and R^{fb2} are preferably a fluorine atom or a linear fluorinated alkyl group having 1 to 4 carbon atoms. Furthermore, R^{fb1} and Rf^{b2} may be bonded to each other to form a ring together with the group (—CF₂—SO₂—N—SO₂—CF₂—) to which they are bonded, in which case the group obtained by R^{fb1} and R^{fb2} bonding to each other is preferably a fluorinated ethylene group or a fluorinated propylene group.

In the general formula (c1-3), R^{fc1}, R^{fc2}, and R^{fc3} each independently represent a fluorine atom, or a hydrocarbyl group having 1 to 40 carbon atoms and optionally containing a heteroatom. The hydrocarbyl group may be saturated or unsaturated and may be linear, branched, or cyclic. Specific examples thereof include the same ones as those exemplified as the hydrocarbyl group represented by R^{fa1} in the general formula (c1-1-1). R^{fc1}, R^{fc2}, and R^{fc3} is preferably a fluorine atom or a linear fluorinated alkyl group having 1 to 4 carbon atoms. Furthermore, R^{fc1} and R^{fc2} may be bonded to each other to form a ring together with the group (—CF₂—SO₂—C—SO₂—CF₂—) to which they are bonded, in which case the group obtained by R^{fc1} and R^{fc2} bonding to each other is preferably a fluorinated ethylene group or a fluorinated propylene group.

In the general formula (c1-4), R^{fd} represents a hydrocarbyl group having 1 to 40 carbon atoms and optionally containing a heteroatom. The hydrocarbyl group may be saturated or unsaturated and may be linear, branched, or cyclic. Specific examples thereof include the same ones as those exemplified as the hydrocarbyl group represented by R^{fa1} in the general formula (c1-1-1).

Specific examples of the anion represented by the general formula (c1-4) include, but are not limited to, the following.

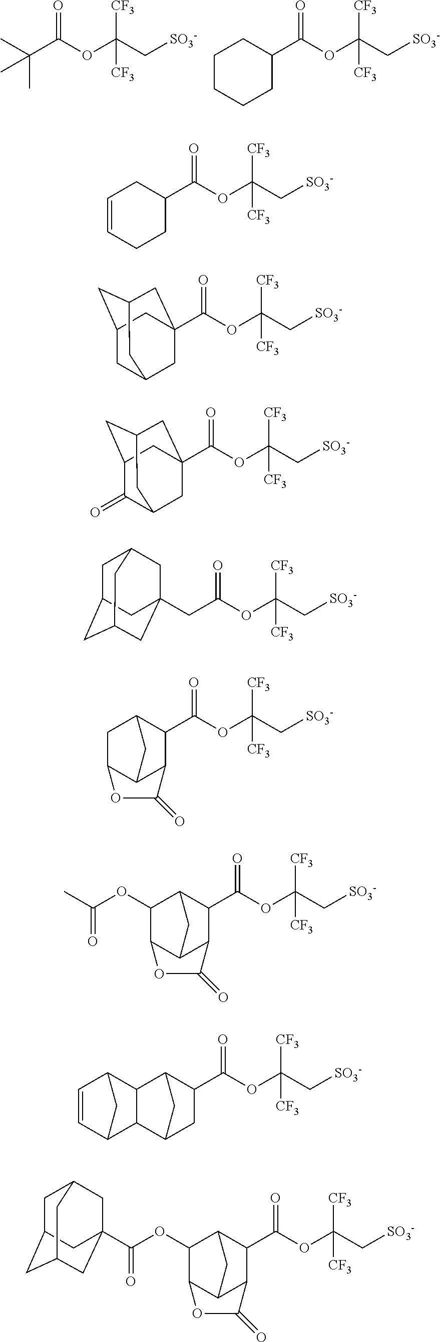

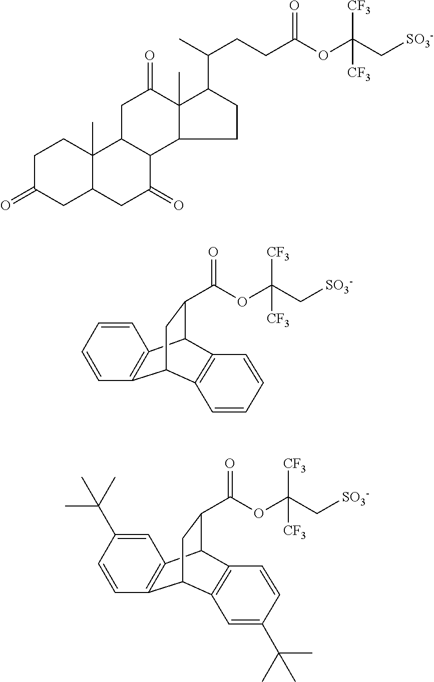

Further examples of the non-nucleophilic counterion include an anion having an aromatic ring substituted with an iodine atom or a bromine atom. Specific examples of such an anion include those represented by the following general formula (c1-5).

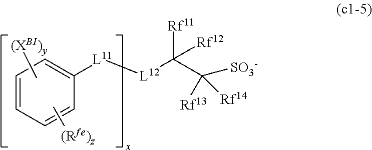

In the general formula (c1-5), “x” represents 1, 2 or 3. “y” represents 1, 2, 3, 4 or 5. “z” represents 0, 1, 2 or 3. However, 1≤y+z≤5. “y” is preferably 1, 2 or 3, and more preferably 2 or 3. “z” is preferably 0, 1 or 2.

In the general formula (c1-5), X^{BI} is an iodine atom or a bromine atom, and when “x” and/or “y” are 2 or more, they may be the same as or different from each other.

In the general formula (c1-5), L¹¹ represents a single bond, an ether bond, an ester bond, or a saturated hydrocarbylene group having 1 to 6 carbon atoms and optionally containing an ether bond or an ester bond. The saturated hydrocarbylene group may be linear, branched, or cyclic.

In the general formula (c1-5), L¹² represents a single bond or a divalent linking group having 1 to 20 carbon atoms when “x” is 1, and represents an (x+1)-valent linking group having 1 to 20 carbon atoms when “x” is 2 or 3, and the linking group may contain an oxygen atom, a sulfur atom, or a nitrogen atom.

In the general formula (c1-5), R^{fe} represents a hydroxy group, a carboxy group, a fluorine atom, a chlorine atom, a bromine atom, an amino group, or a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, and optionally containing a fluorine atom, a chlorine atom, a bromine atom, a hydroxy group, an amino group, or an ether bond, or —N(R^{feA}) (R^{feB}), —N(R^{feC})—C(═O)—R^{feD}, or —N(R^{feC})—C(═O)—O—R^{feD}. R^{feA} and R^{feB} each independently represent a hydrogen atom or a saturated hydrocarbyl group having 1 to 6 carbon atoms. R^{feC} represents a hydrogen atom or a saturated hydrocarbyl group having 1 to 6 carbon atoms, and optionally containing a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{feD} represents an aliphatic hydrocarbyl group having 1 to 16 carbon atoms, an aryl group having 6 to 12 carbon atoms, or an aralkyl group having 7 to 15 carbon atoms, and optionally containing a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When “x” and/or “z” are 2 or more, each R^{fe} may be the same as or different from each other.

Of these, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, etc.

In the general formula (c1-5), Rf¹¹ to Rf¹⁴ each independently represent a hydrogen atom, a fluorine atom, or a trifluoromethyl group, provided that at least one of them is a fluorine atom or a trifluoromethyl group. Furthermore, Rf¹¹ and Rf¹² may combine to form a carbonyl group. In particular, Rf¹³ and Rf¹⁴ are both preferably a fluorine atom.

Specific examples of the anion represented by the general formula (c1-5) include, but are not limited to, the following. Note that in the following formulae, X^{BI} is as defined above.

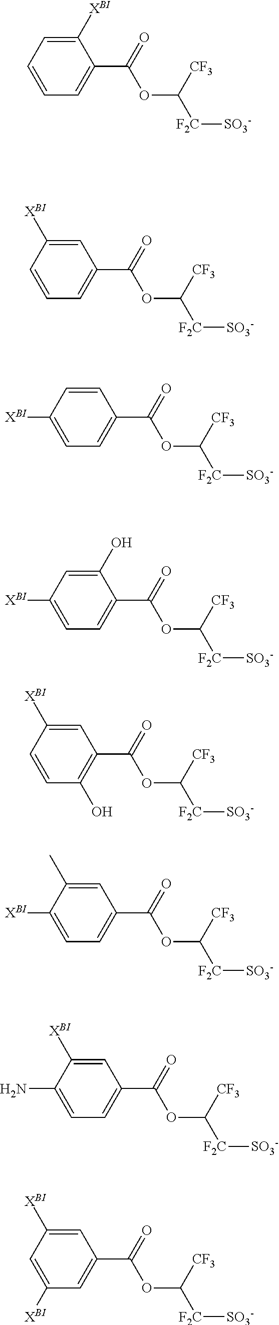

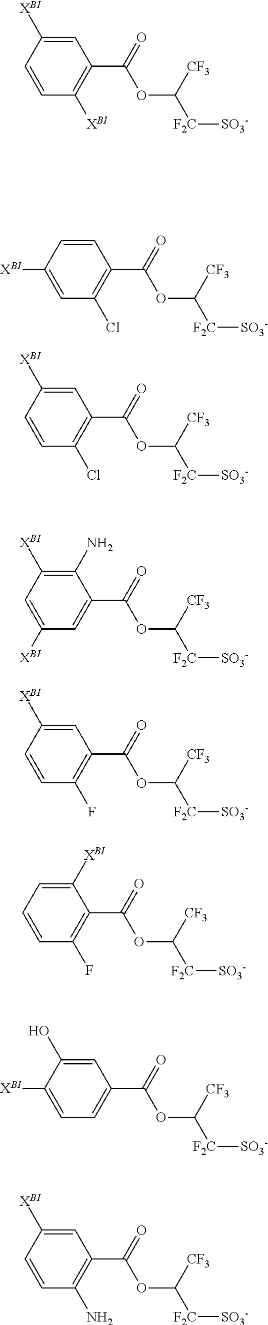

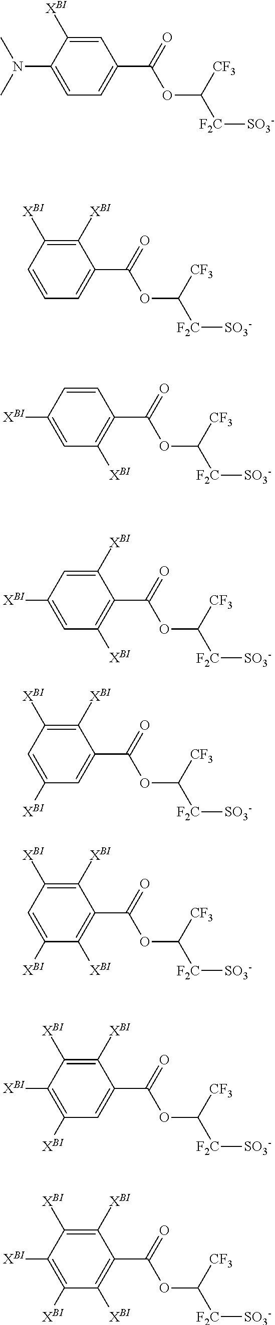

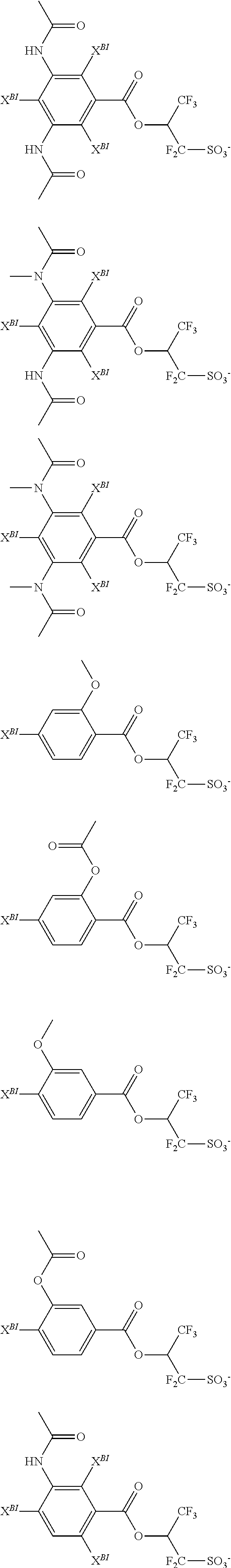

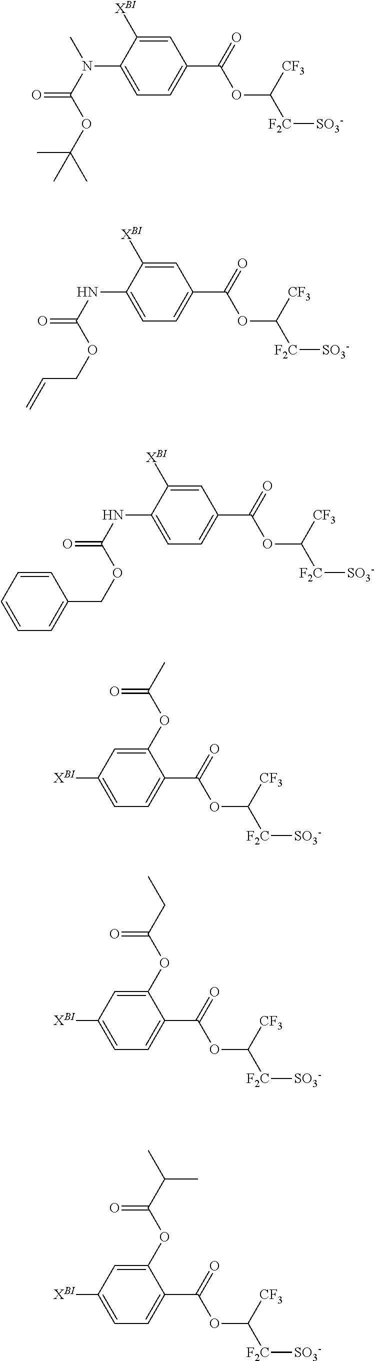

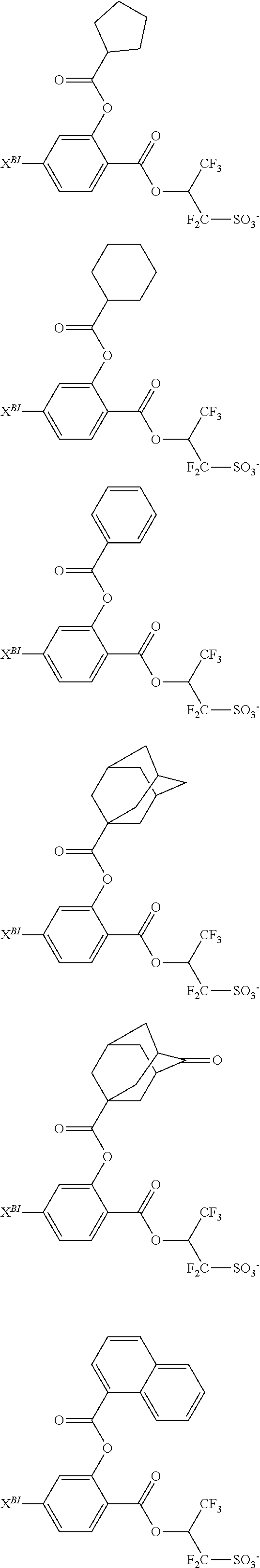

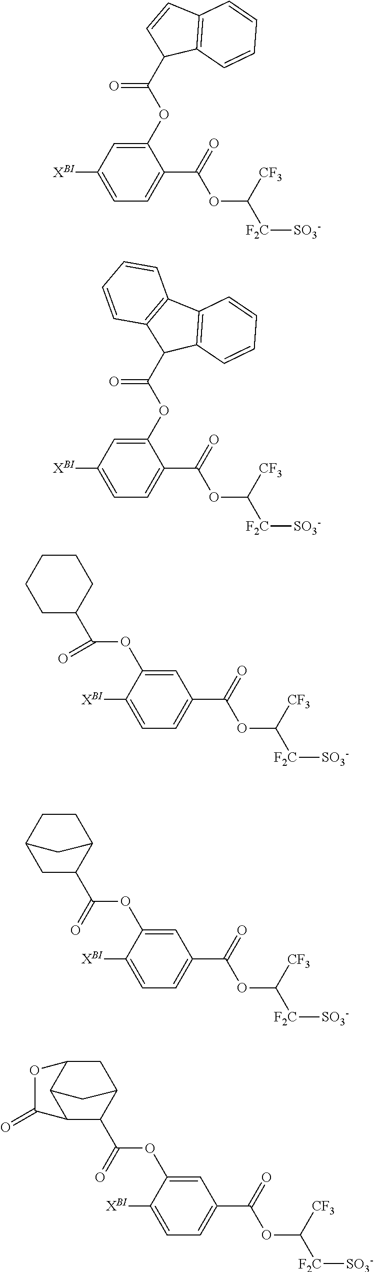

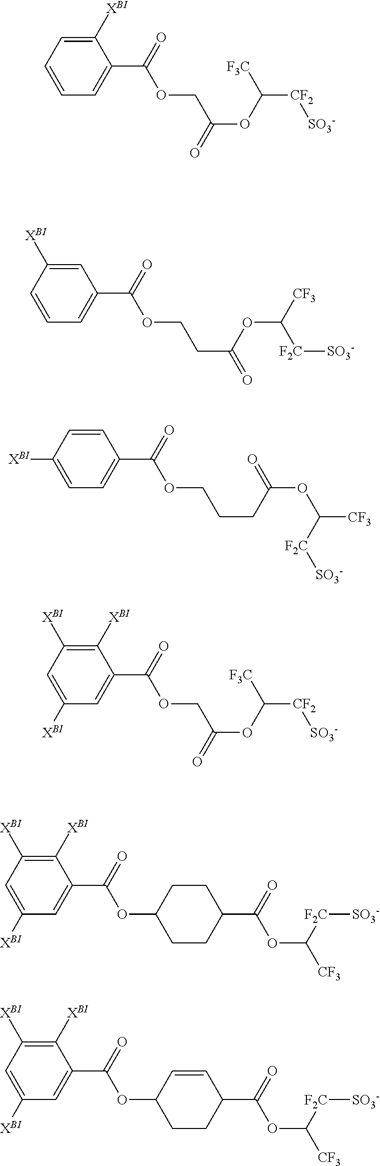

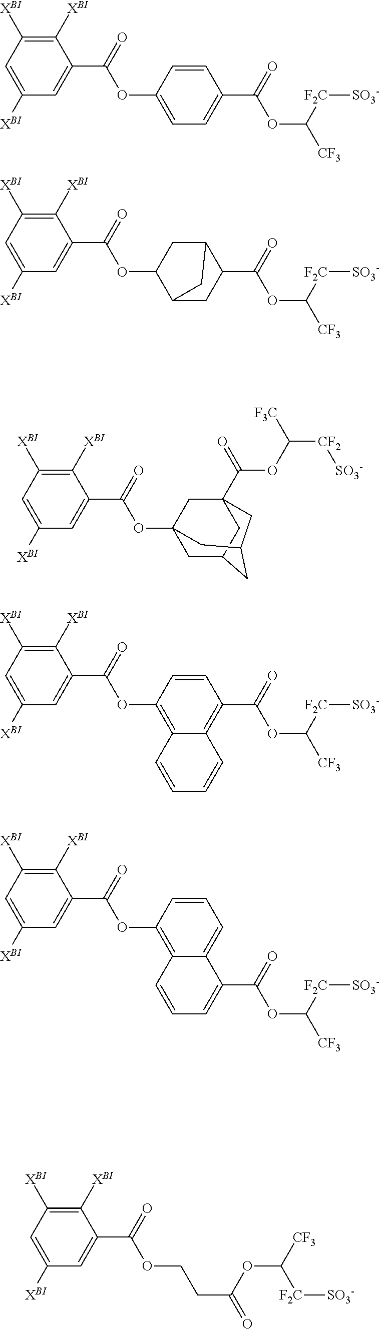

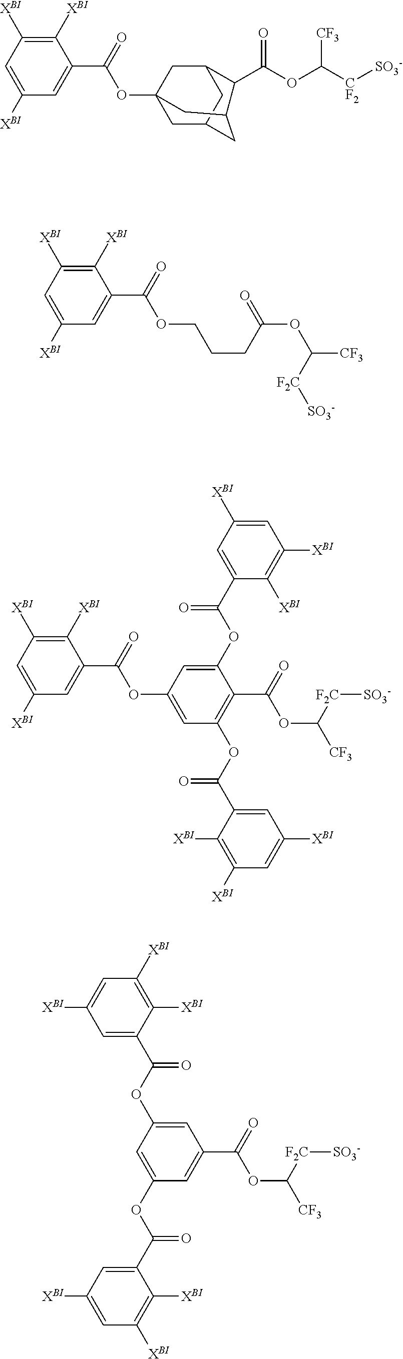

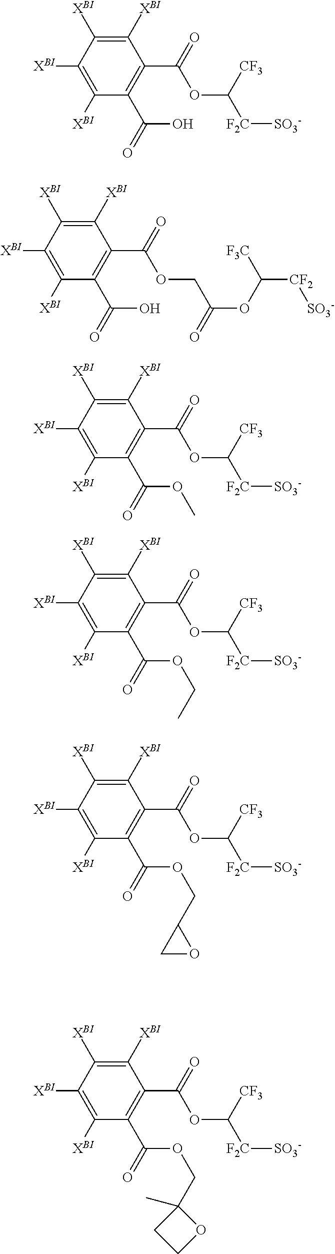

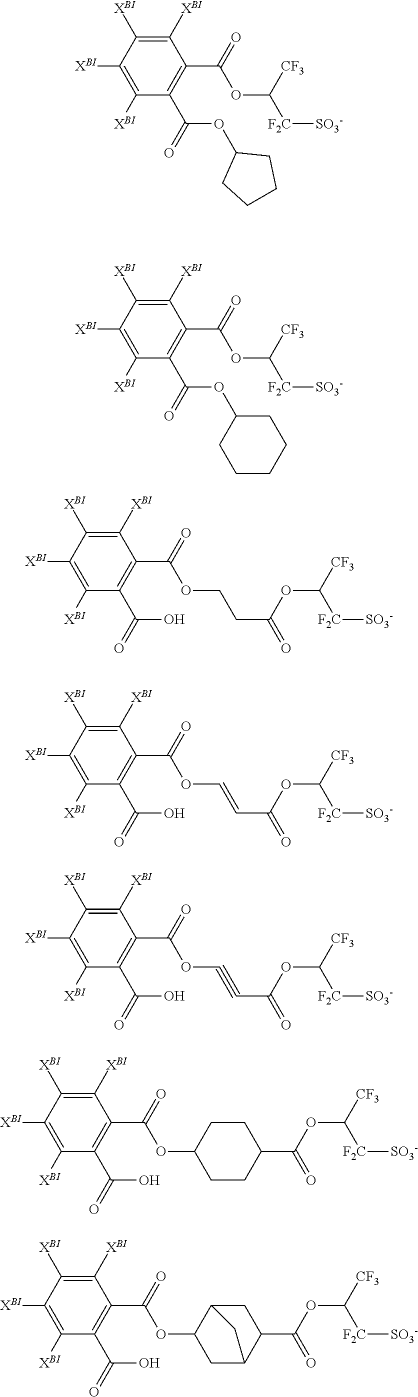

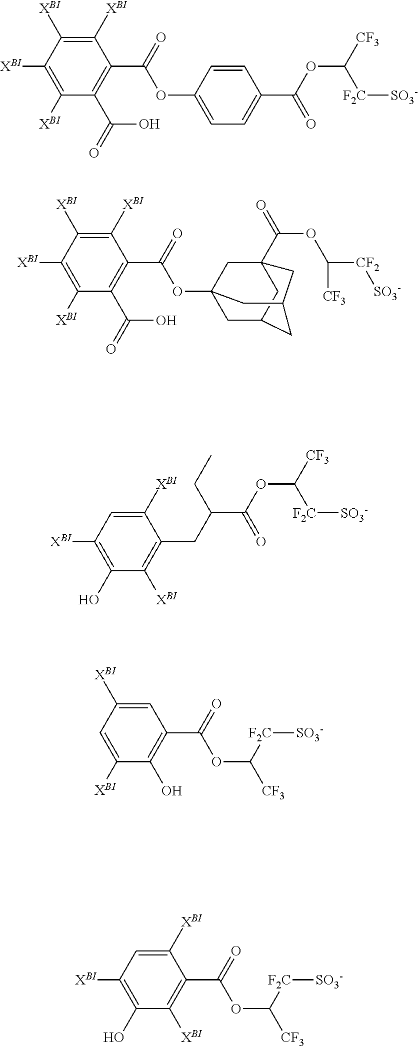

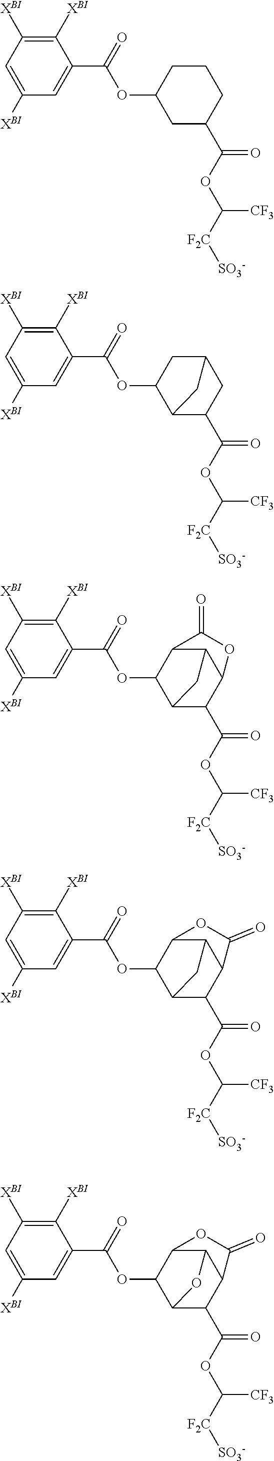

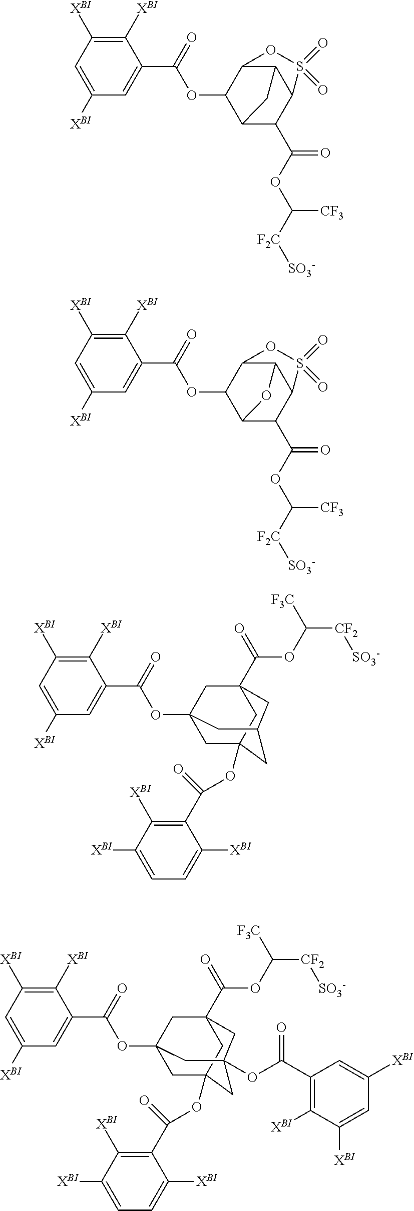

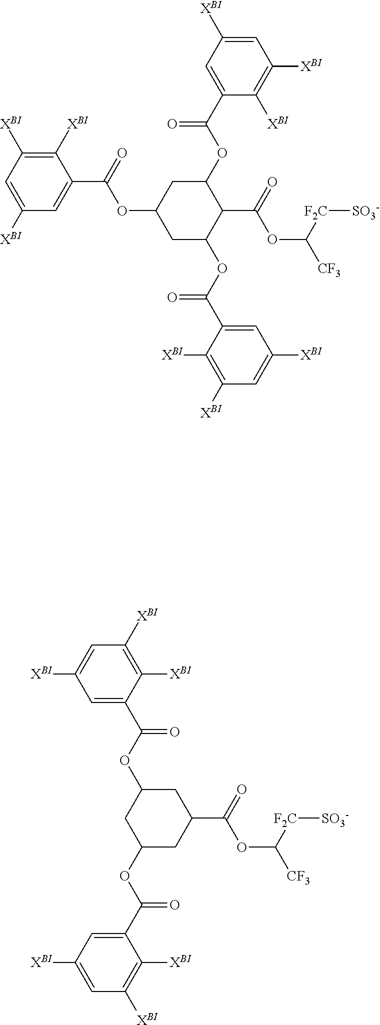

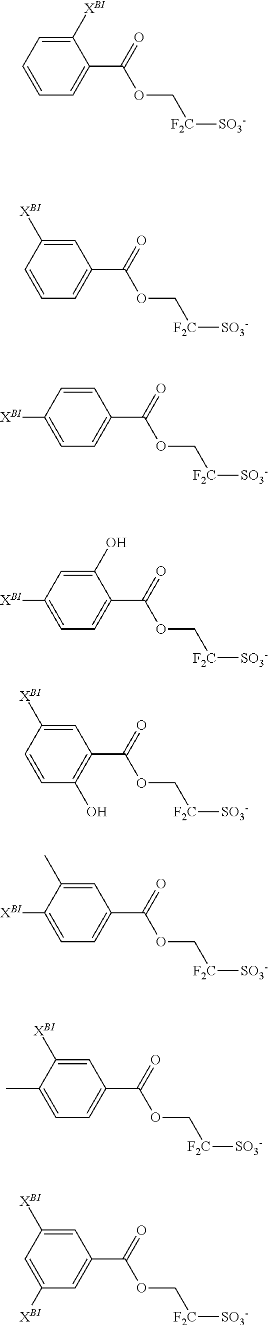

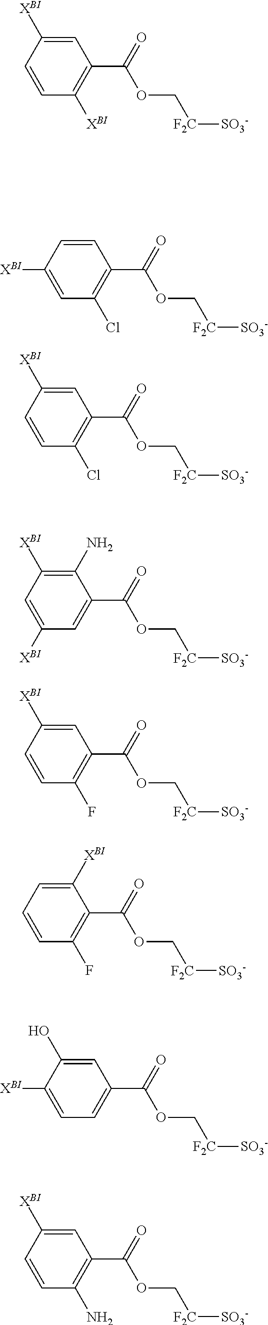

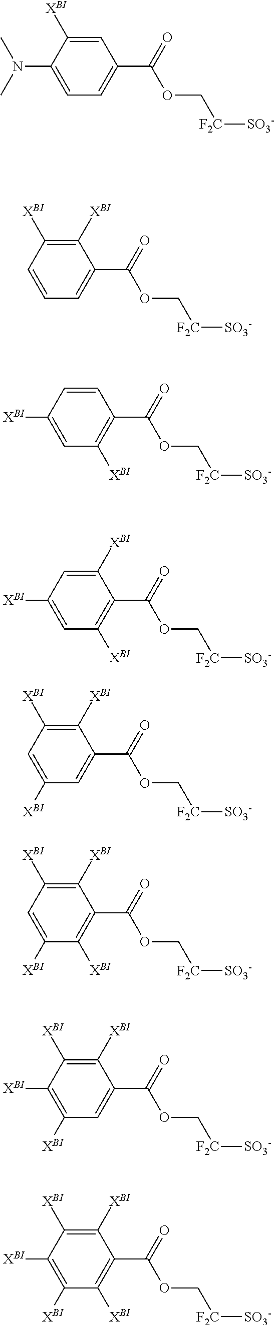

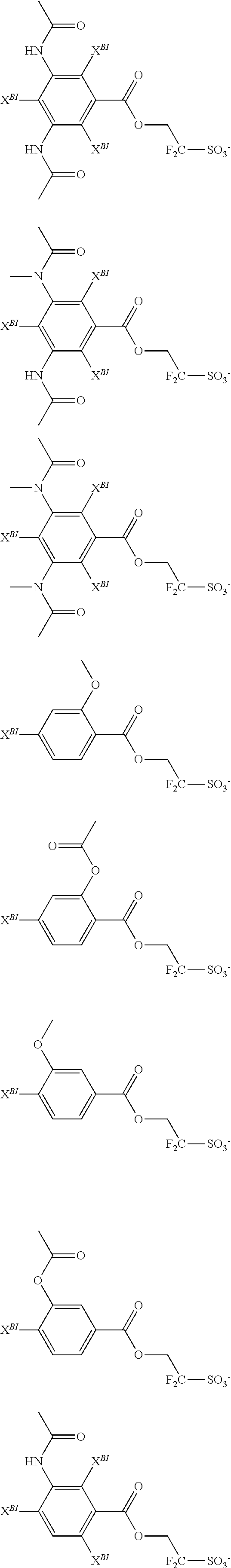

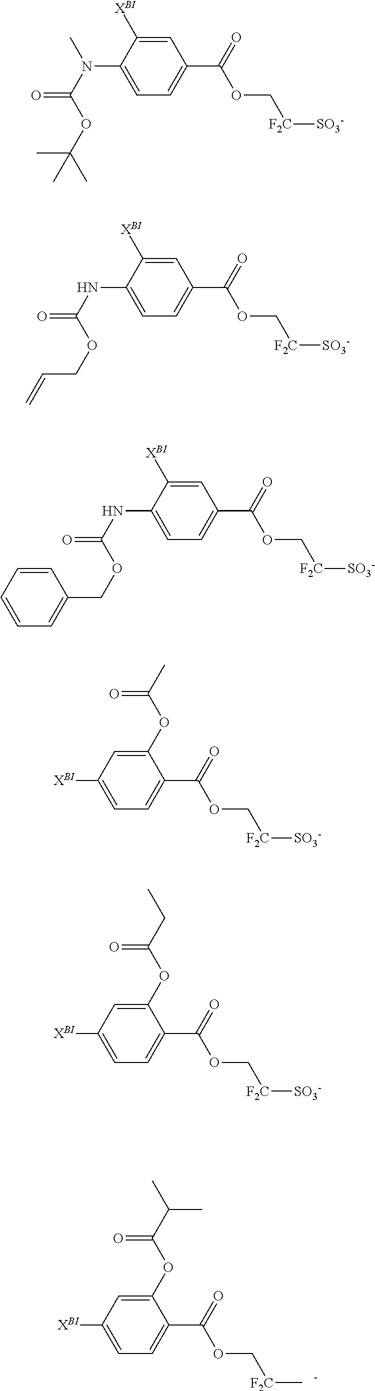

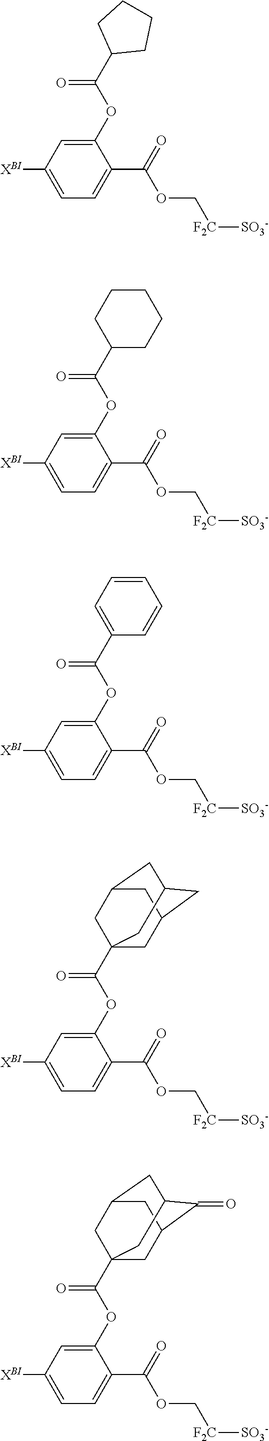

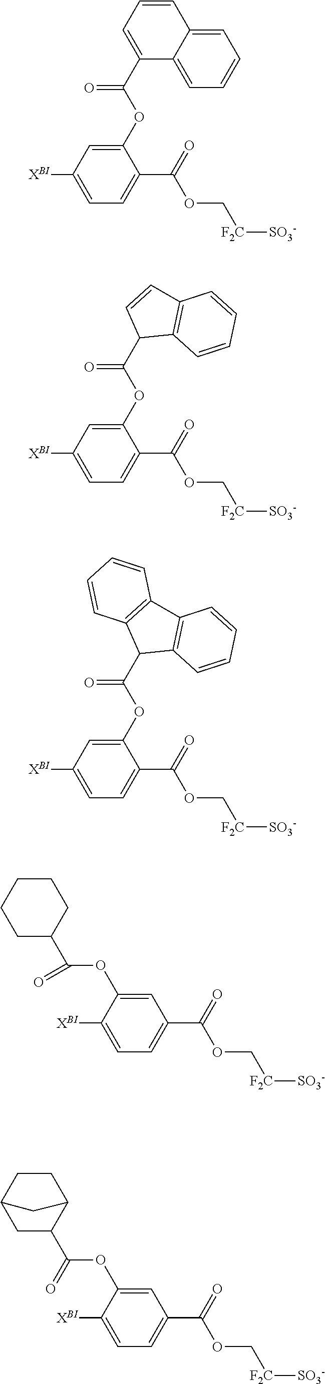

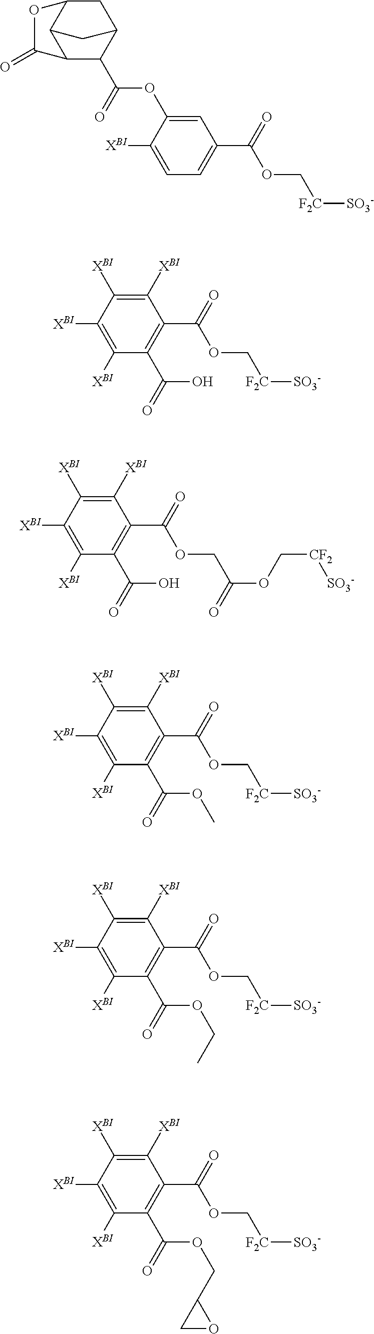

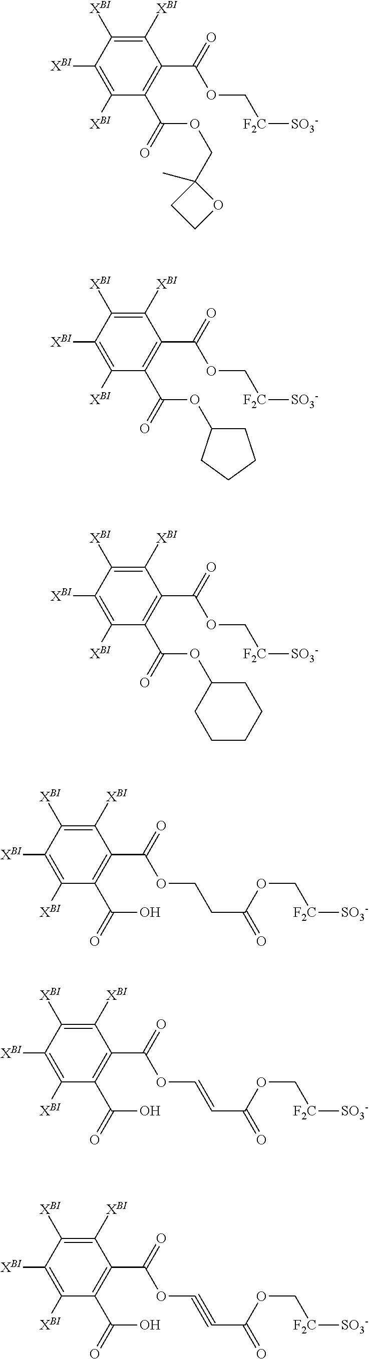

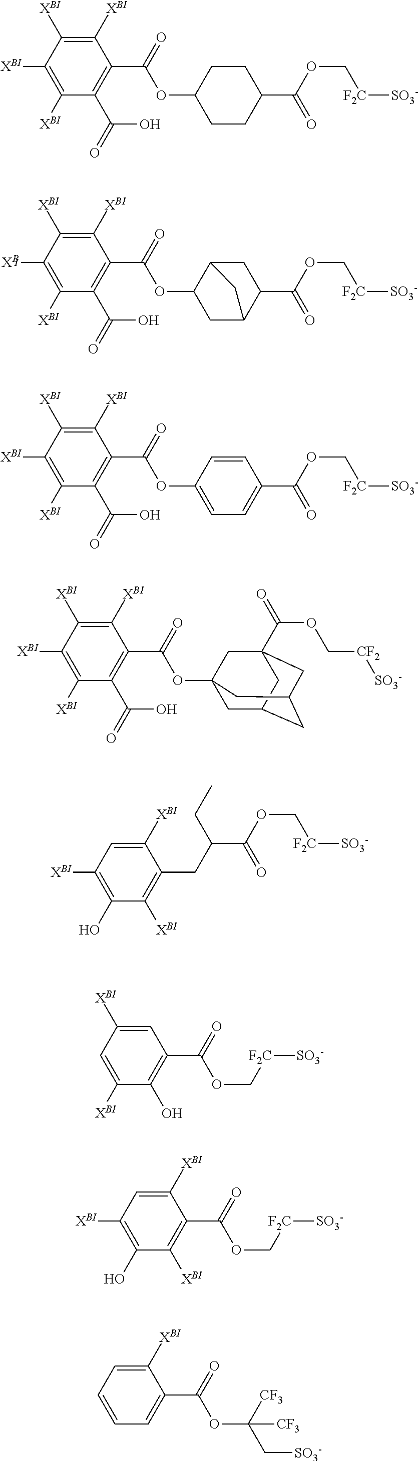

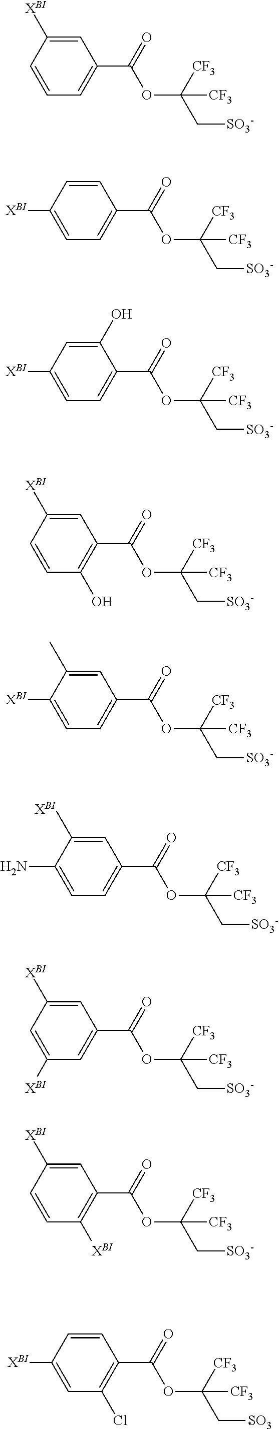

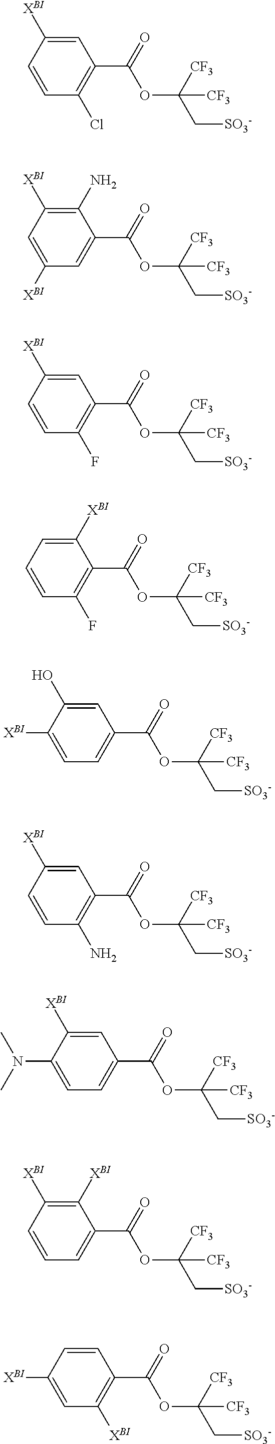

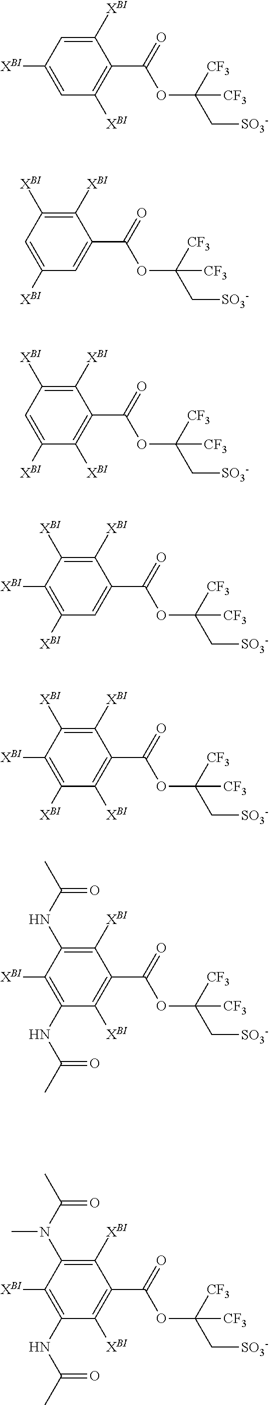

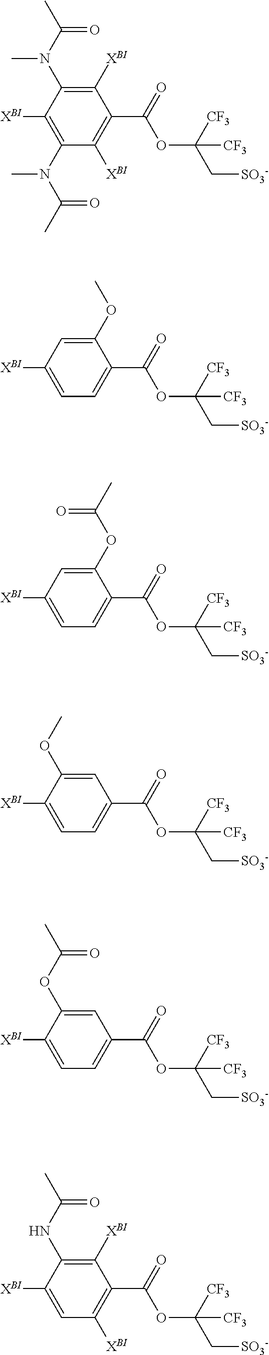

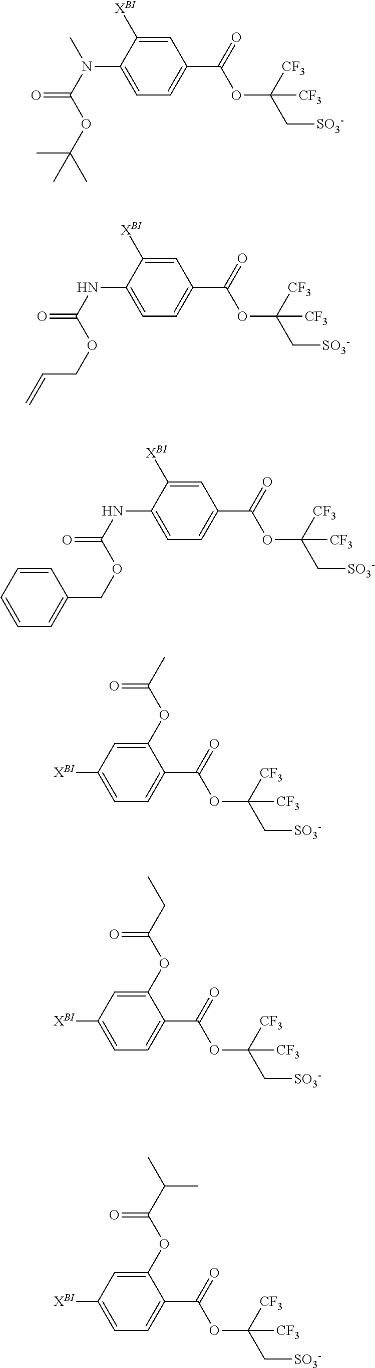

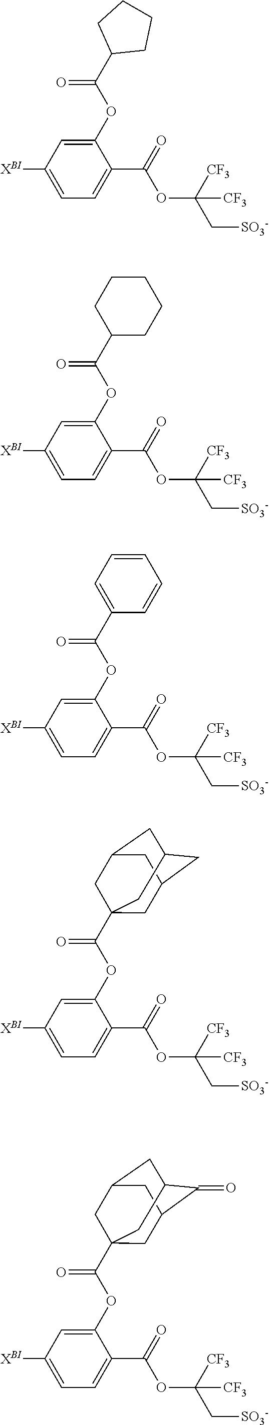

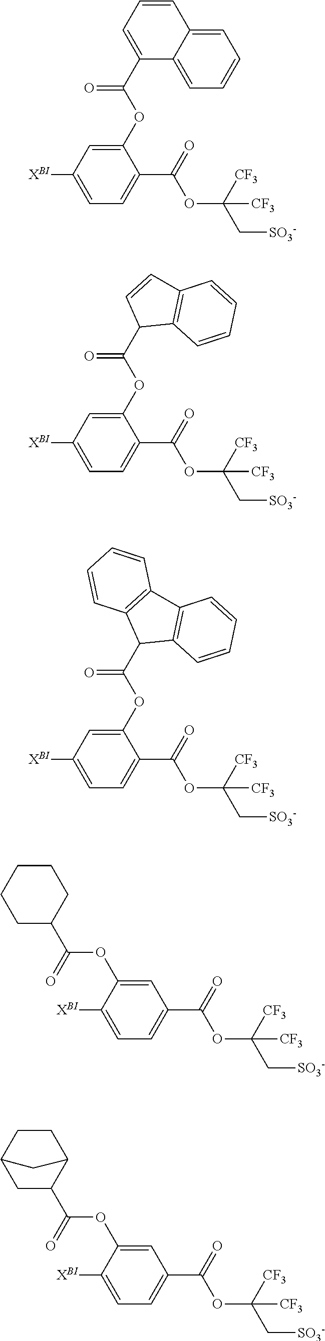

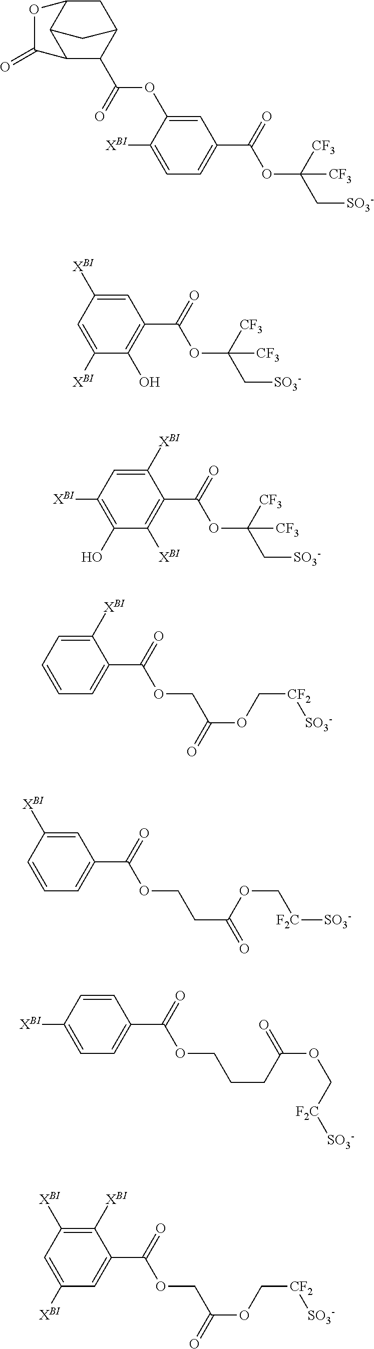

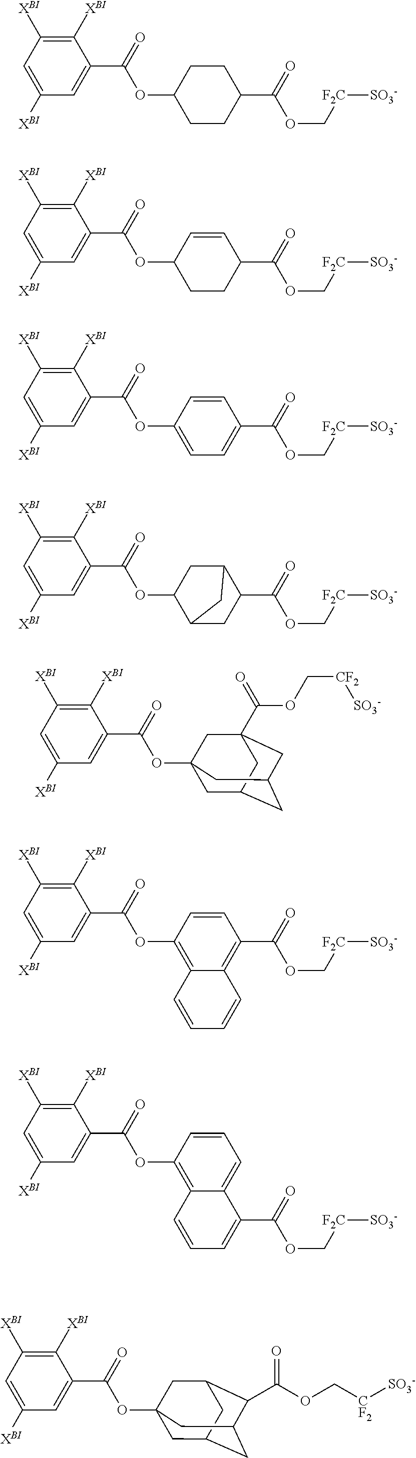

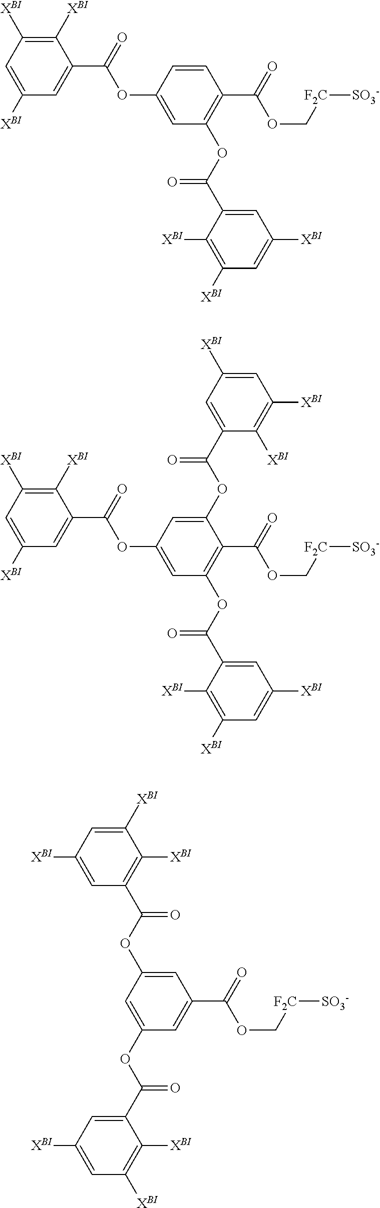

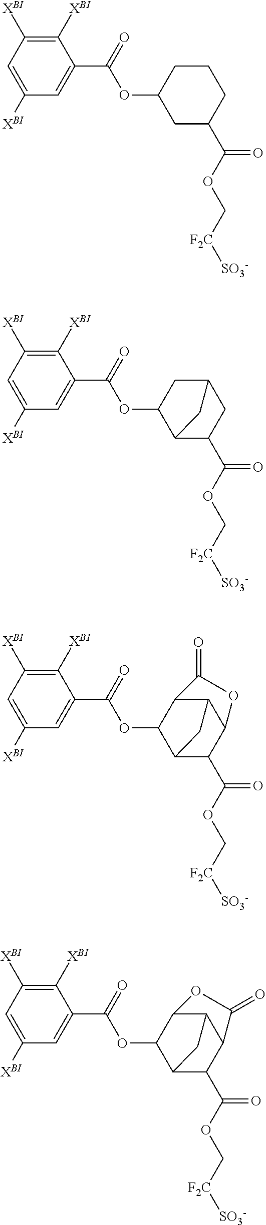

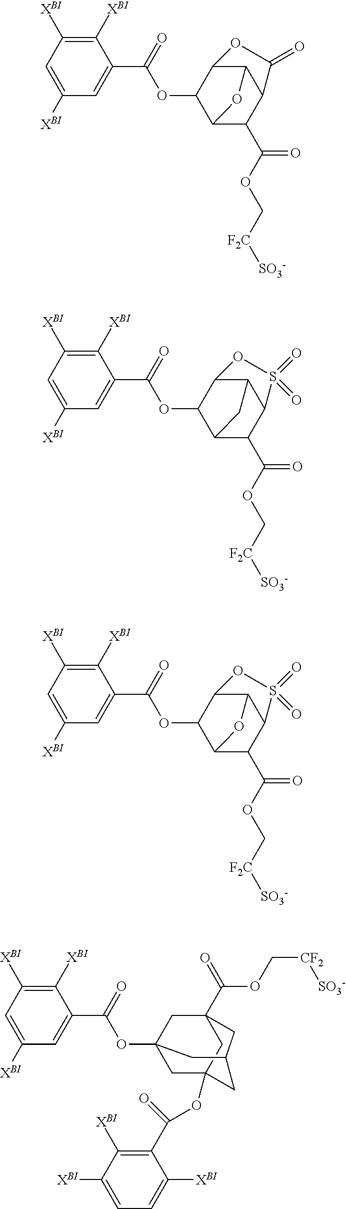

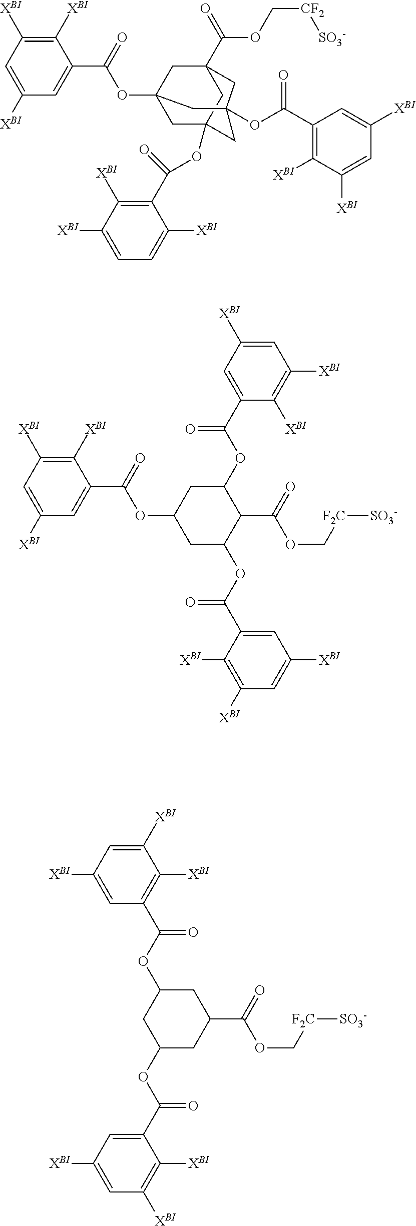

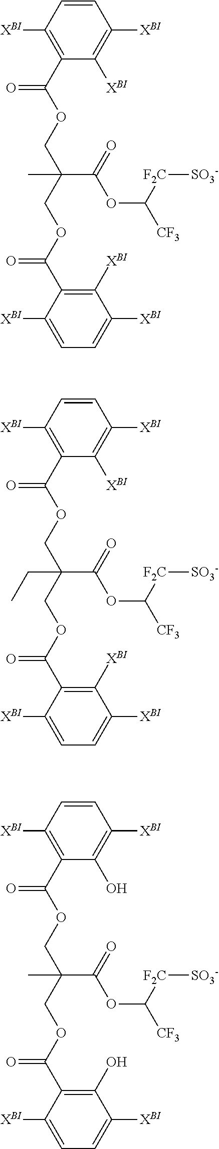

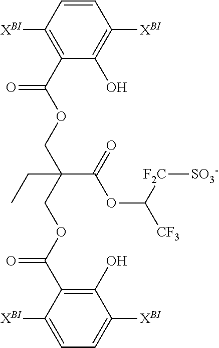

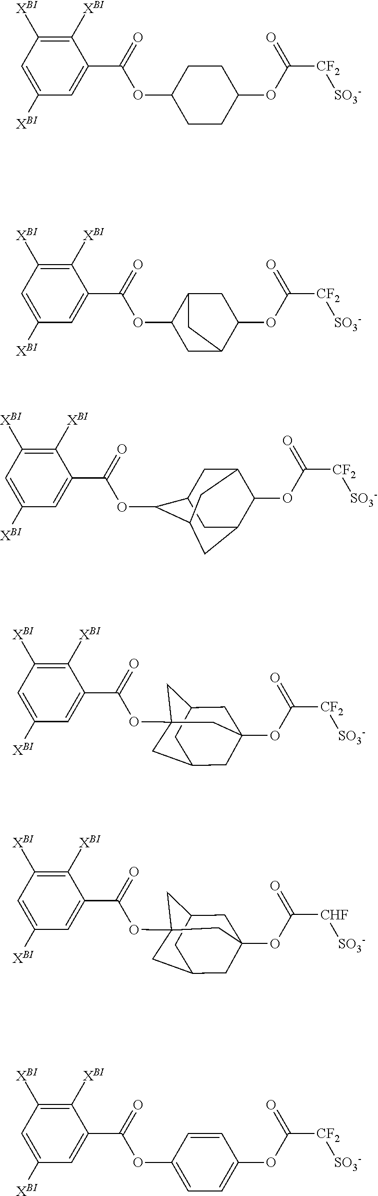

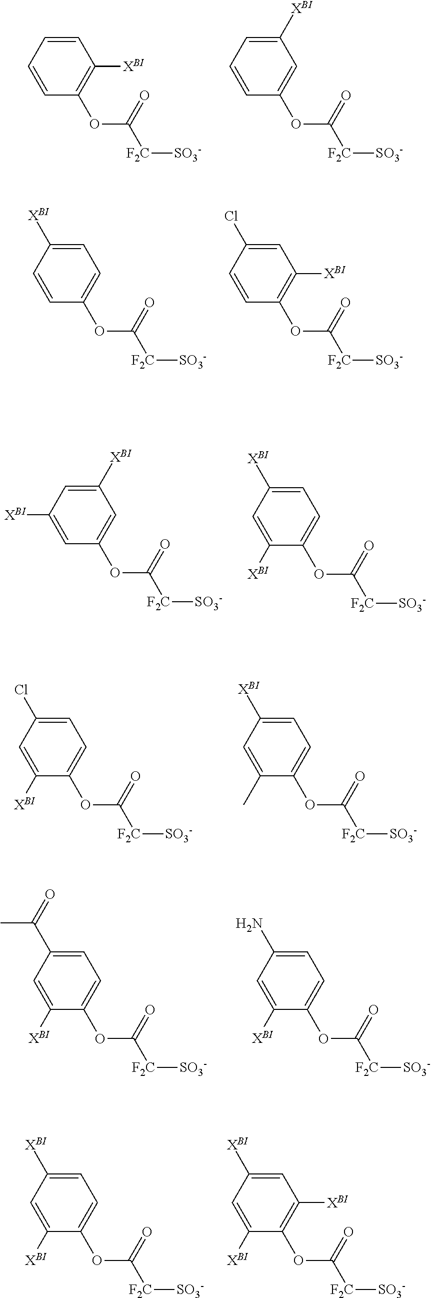

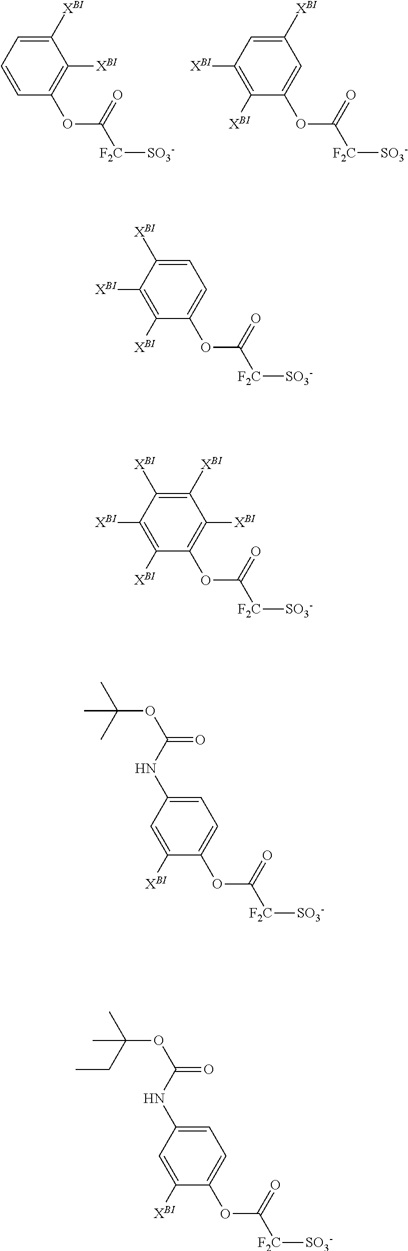

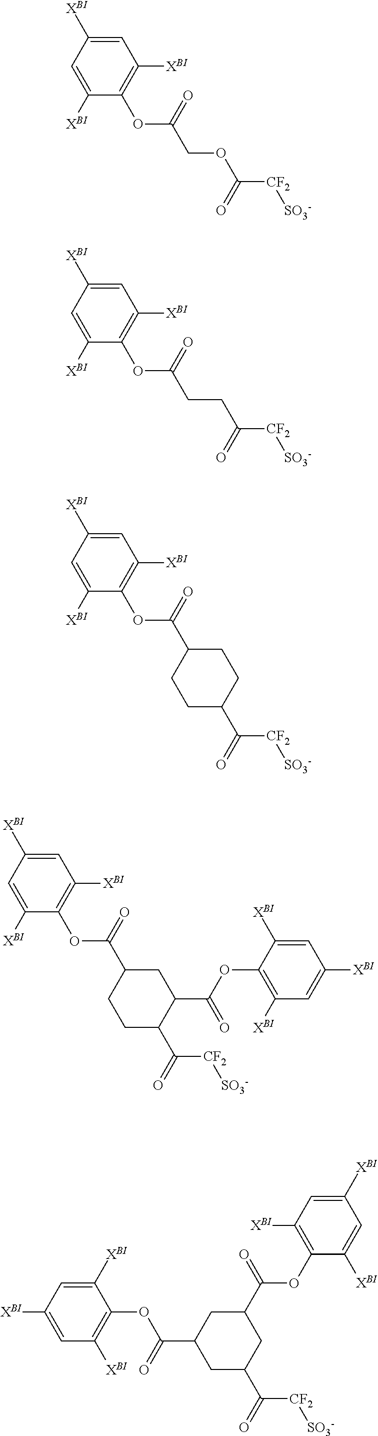

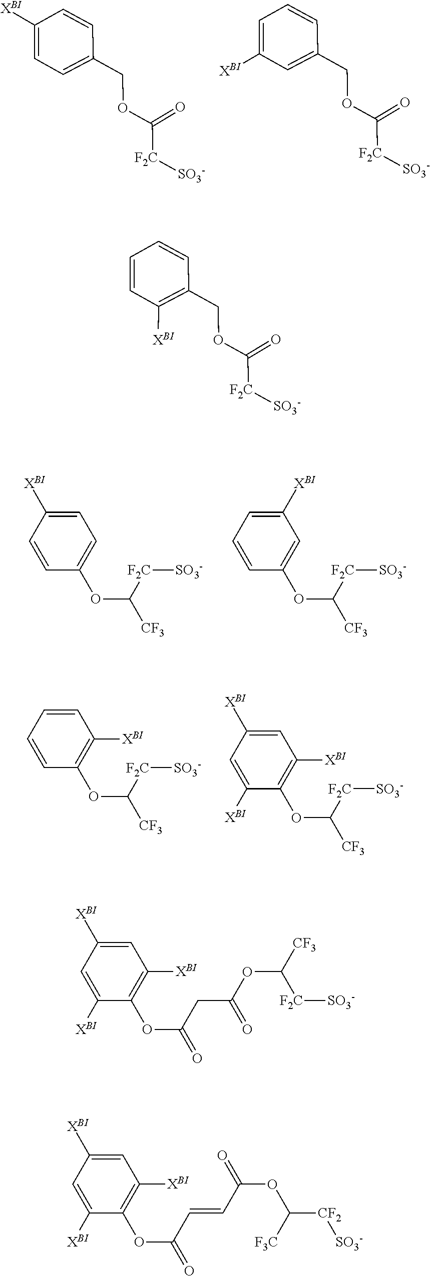

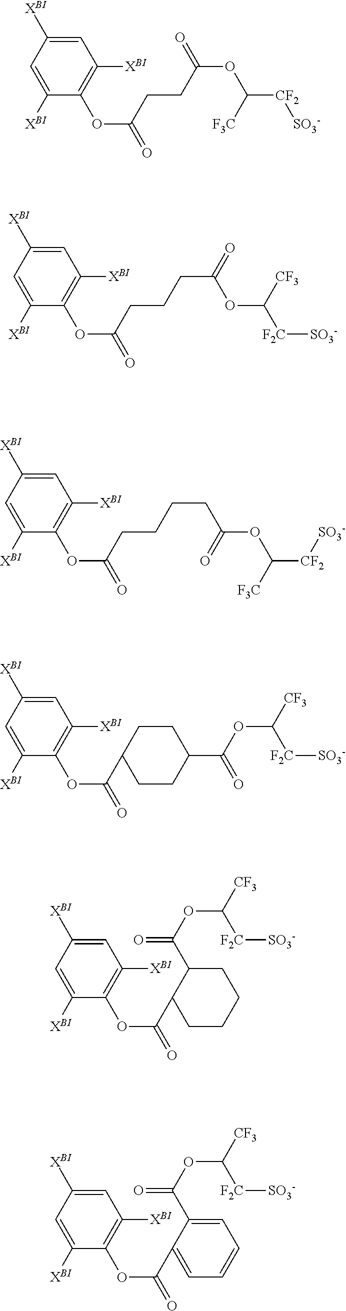

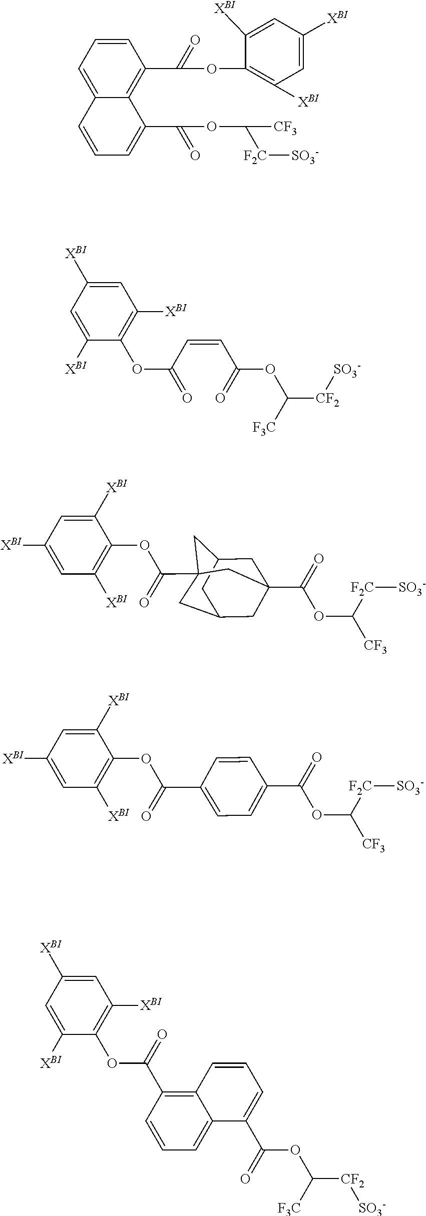

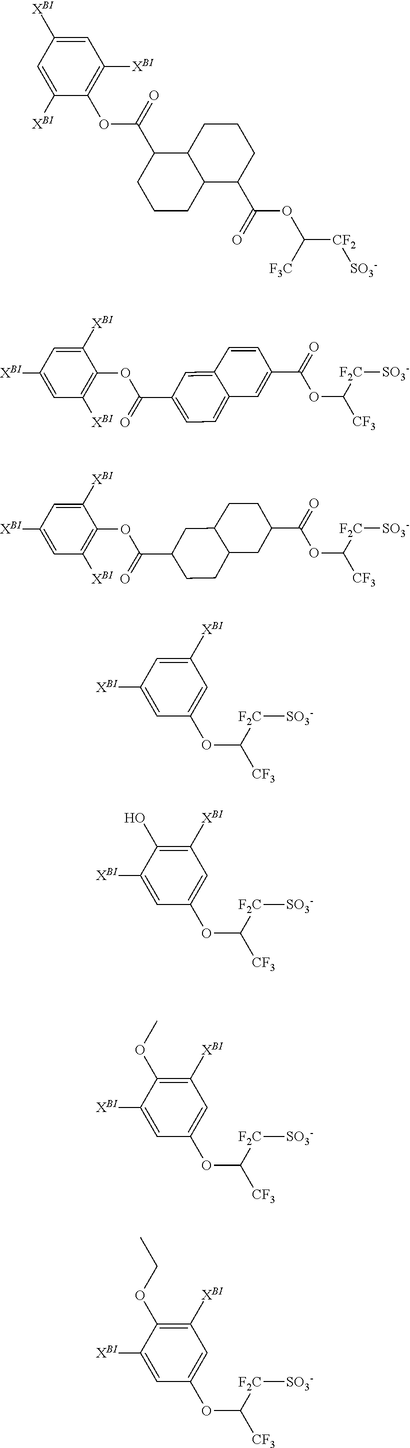

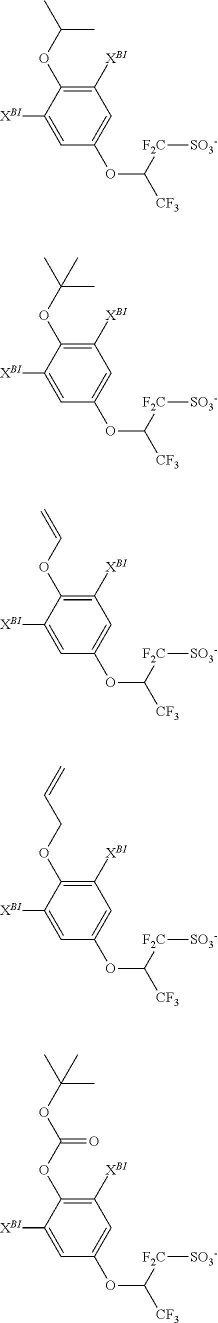

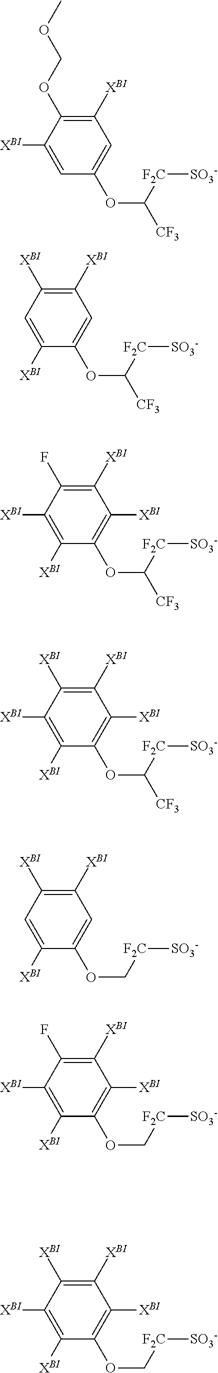

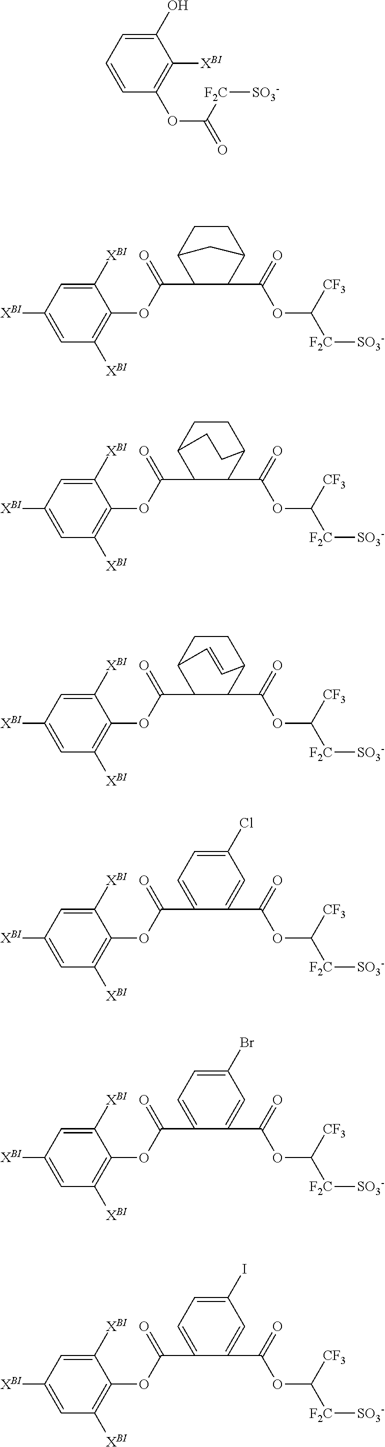

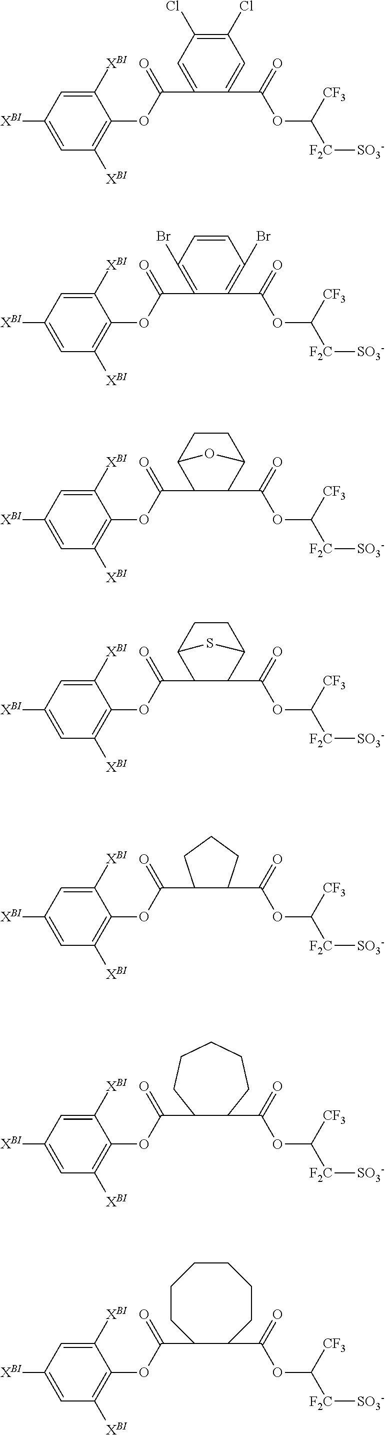

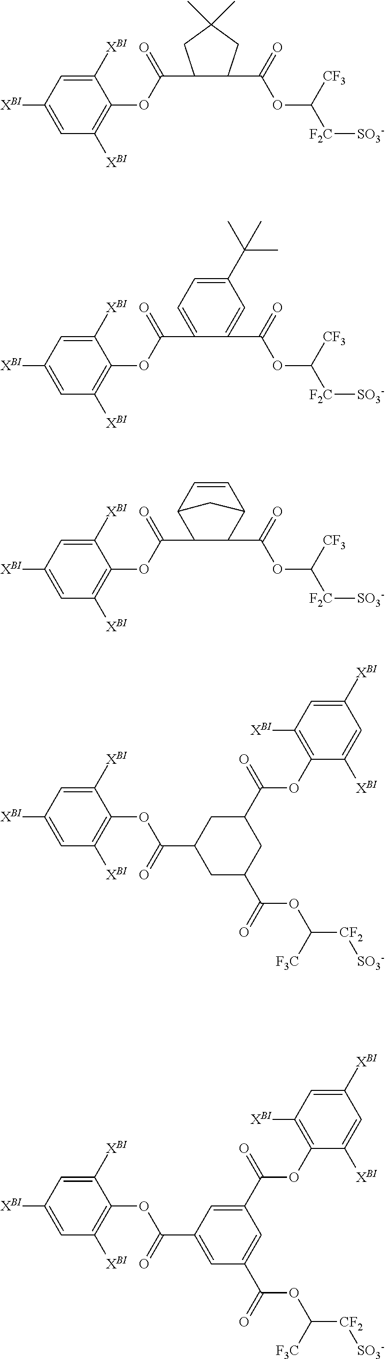

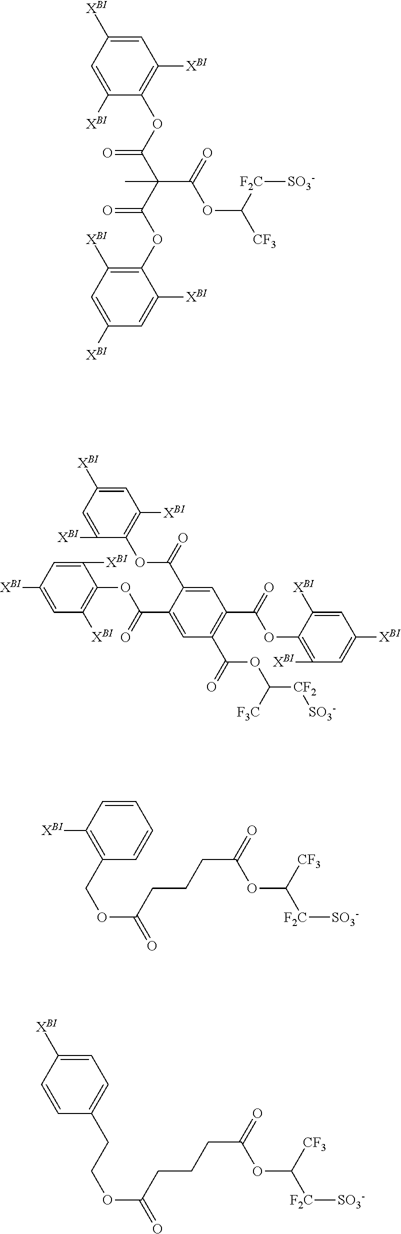

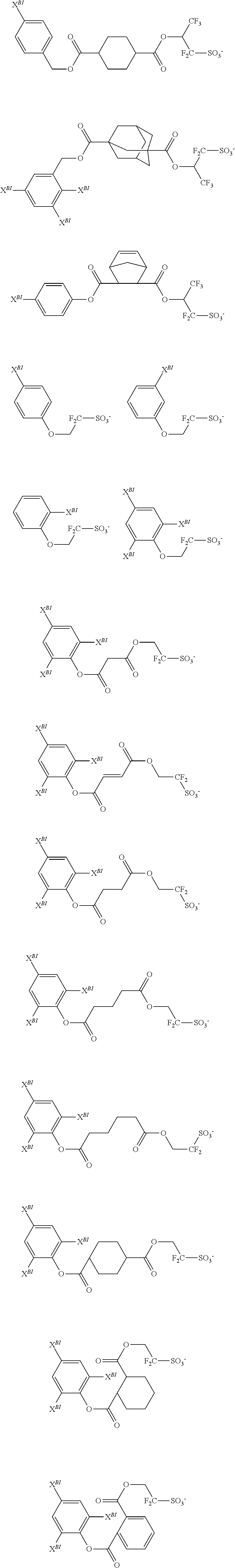

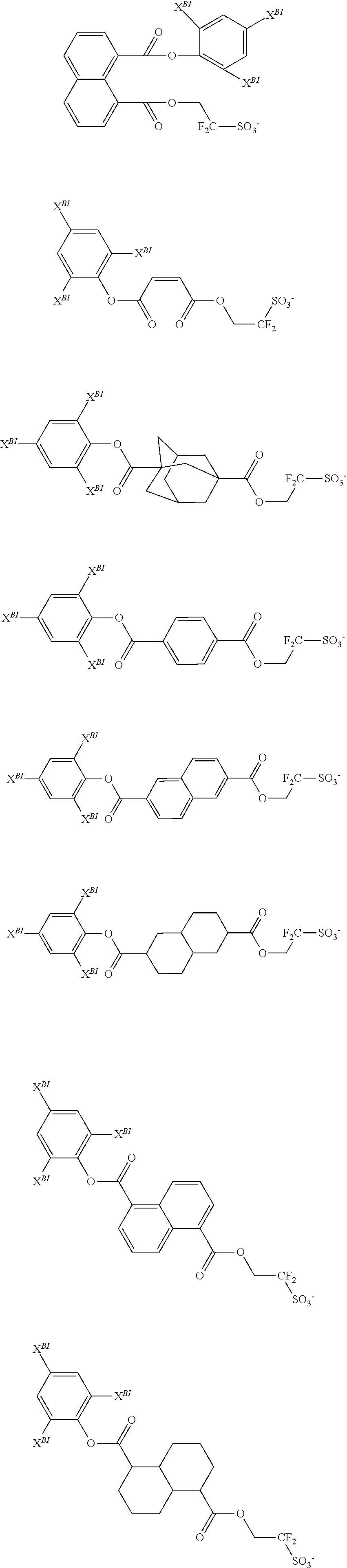

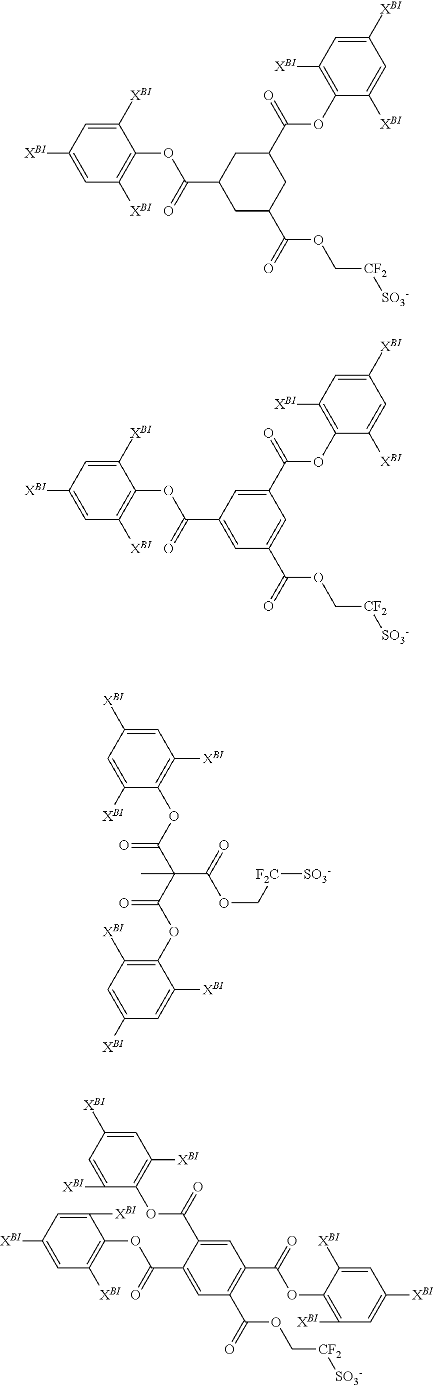

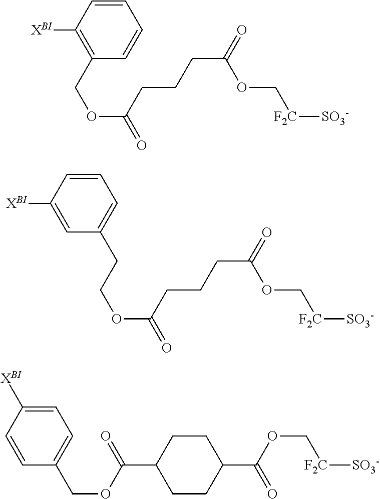

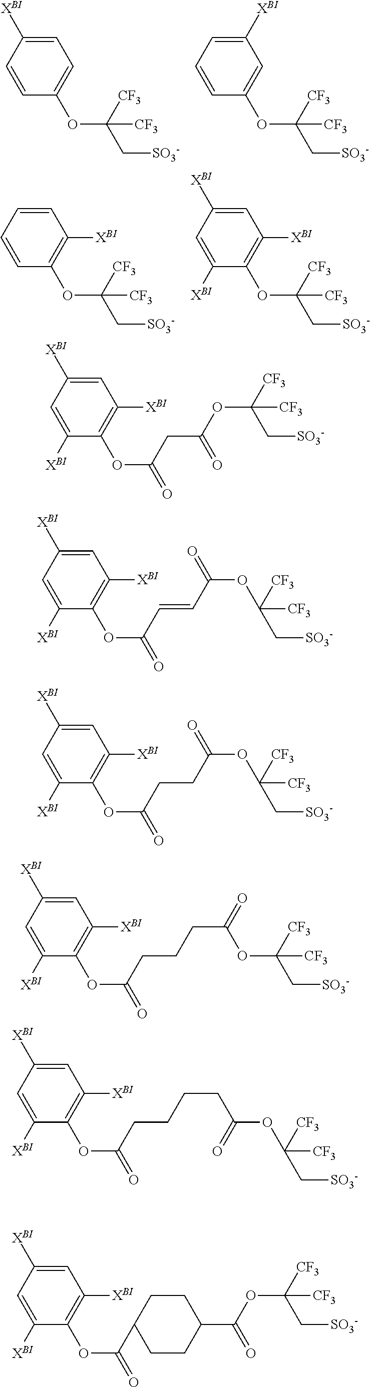

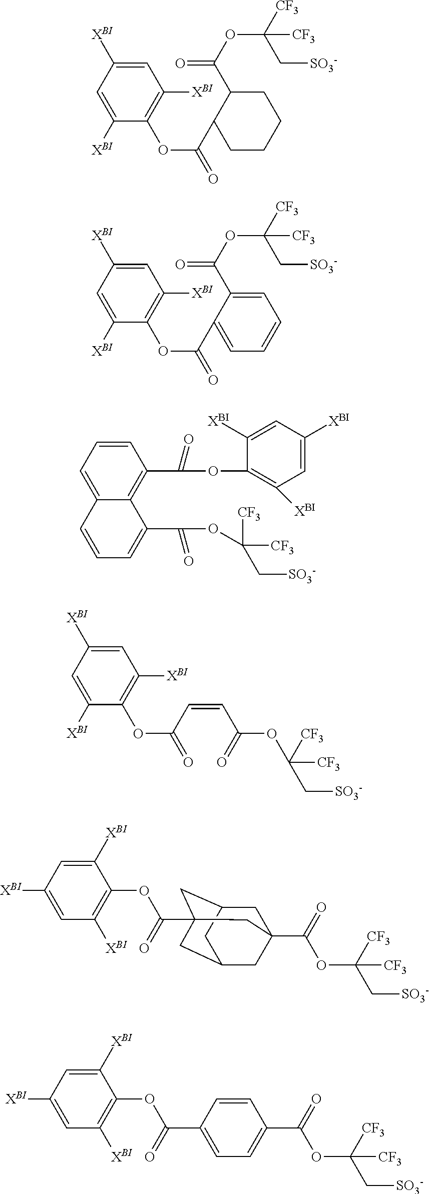

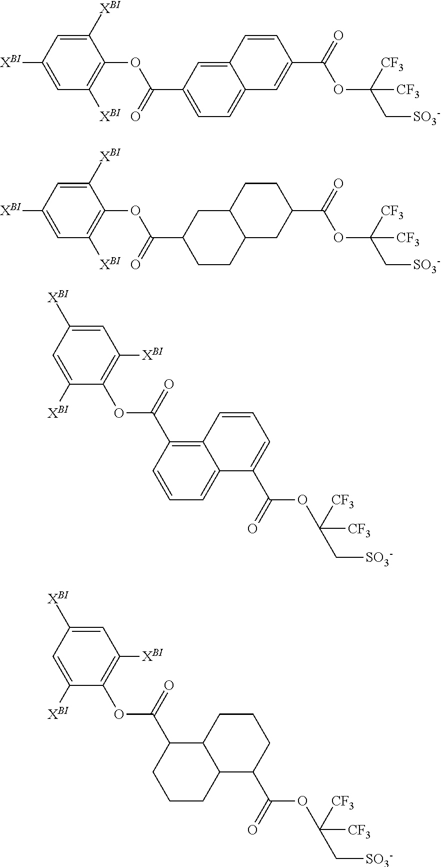

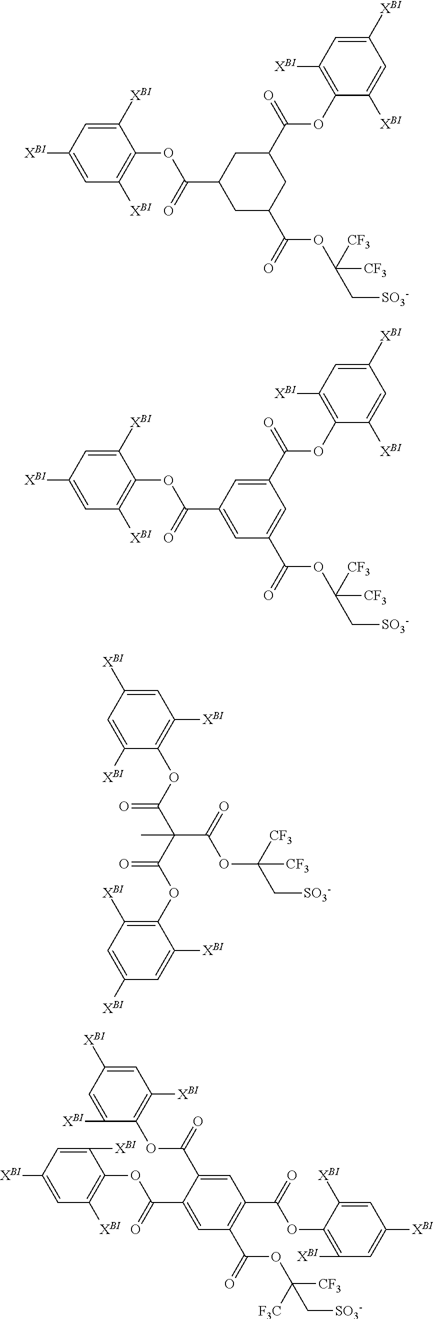

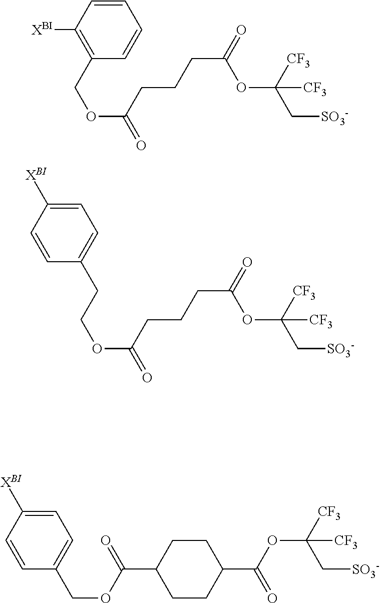

For the non-nucleophilic counterion, the following can also be used: a fluorobenzenesulfonate anion bonded to an aromatic group containing an iodine atom as described in JP6648726B2, an anion having a mechanism of being decomposed by an acid as described in WO2021/200056A1 or JP2021-070692A, an anion having a cyclic ether group as described in JP2018-180525A or JP2021-035935A, and an anion as described in JP2018-092159A.

Furthermore, for the non-nucleophilic counterion, the following can also be used: an anion of a bulky benzenesulfonate derivative not containing a fluorine atom as described in JP2006-276759A, JP2015-117200A, JP2016-065016A, and JP2019-202974A, and a benzenesulfonate anion and an alkylsulfonate anion not containing a fluorine atom bonded to an aromatic group containing an iodine atom as described in JP6645464B2.

Furthermore, for the non-nucleophilic counterion, the following can also be used: an anion of bis-sulfonic acid as described in JP2015-206932A, an anion of having a sulfonic acid on one side and having a sulfonamide or a sulfonimide a different one on the other side as described in WO2020/158366A1, or an anion having a sulfonic acid on one side and a carboxylic acid on the other side as described in JP2015-024989A.

In the general formulae (c2) and (c3), “d1” and “d2” each independently represent an integer from 0 to 3, and are preferably 1.

In the general formula (c4), “e1” represents 0 or 1. “e2” represents an integer from 0 to 4. “e3” represents an integer from 0 to 4. However, when “e1” is 0, 0≤e2+e3≤4, and when “e1” is 1, 0≤e2+e3≤6.

In the general formulae (c2), (c3), and (c4), L¹ represents a single bond, an ether bond, an ester bond, a carbonyl group, a sulfonate ester bond, a sulfonamide bond, a carbonate bond, or a carbamate bond. Among these, from the viewpoint of synthesis, an ether bond, an ester bond, or a carbonyl group is preferable, and an ester bond or a carbonyl group is more preferable.

In the general formula (c2), Rf¹ and Rf² each independently represent a fluorine atom or a fluorinated saturated hydrocarbyl group having 1 to 6 carbon atoms. Of these, in order to increase the acid strength of the generated acid, Rf¹ and Rf² are both preferably a fluorine atom. Rf³ and Rf⁴ each independently represent a hydrogen atom, a fluorine atom, or a fluorinated saturated hydrocarbyl group having 1 to 6 carbon atoms. Of these, in order to improve solution solubility, at least one of Rf³ and Rf⁴ is preferably a trifluoromethyl group.

In the general formula (c3), Rf⁵ and Rf⁶ each independently represent a hydrogen atom, a fluorine atom, or a fluorinated saturated hydrocarbyl group having 1 to 6 carbon atoms. However, all Rf⁵ and Rf⁶ cannot simultaneously become a hydrogen atom. Of these, in order to improve solution solubility, at least one of Rf⁵ and Rf⁶ is preferably a trifluoromethyl group.

In the general formula (c4), Rf⁷ represents a fluorine atom, a fluorinated alkyl group having 1 to 6 carbon atoms, a fluorinated alkoxy group having 1 to 6 carbon atoms, or a fluorinated alkylthio group having 1 to 6 carbon atoms. Rf⁷ is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. When “e2” is 2, 3 or 4, each Rf⁷ may be the same as or different from each other.

In the general formula (c4), R⁴³ represents a halogen atom other than a fluorine atom, or a hydrocarbyl group having 1 to 20 carbon atoms and optionally containing a heteroatom. The hydrocarbyl group may be saturated or unsaturated and may be linear, branched, or cyclic. Specific examples thereof include the same ones as those exemplified as the hydrocarbyl group represented by R¹ in the explanation of the general formula (1A), but are not limited to these. Also, when “e3” is 2, 3 or 4, each R⁴³ may be the same as or different from each other.

Also, when “e3” is 2, 3 or 4, a plurality of R⁴³ may be bonded to each other to form a ring together with the carbon atoms to which they are bonded. Specific examples of the ring formed in this case include a cyclopropane ring, a cyclobutane ring, a cyclopentane ring, a cyclohexane ring, a norbornane ring, an adamantane ring, etc. In addition, some or all of the hydrogen atoms of the above-described rings may be substituted with a group containing a heteroatom such as an oxygen atom, a sulfur atom, a nitrogen atom, or a halogen atom, and some of the —CH₂— of the above-described rings may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the anion of the repeating unit-c2 include, but are not limited to, the following. Note that in the following formulae, R^{A} is as defined above, and Me represents a methyl group.

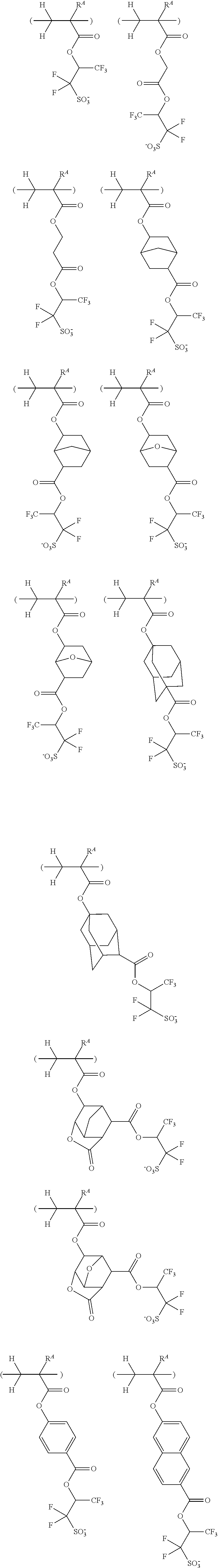

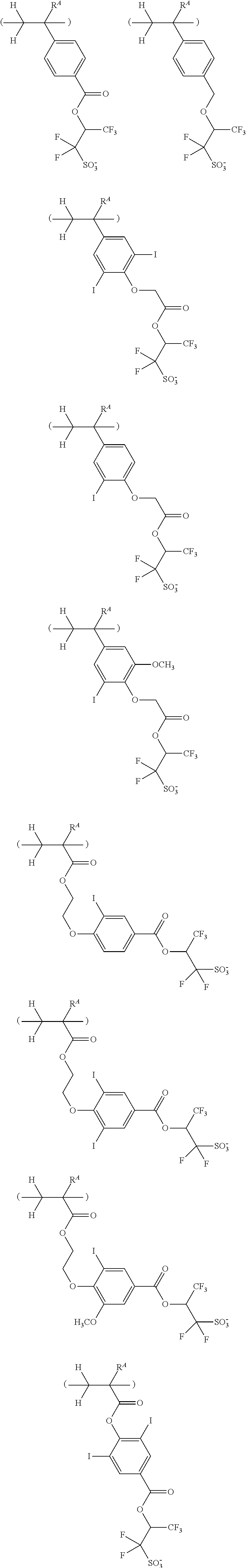

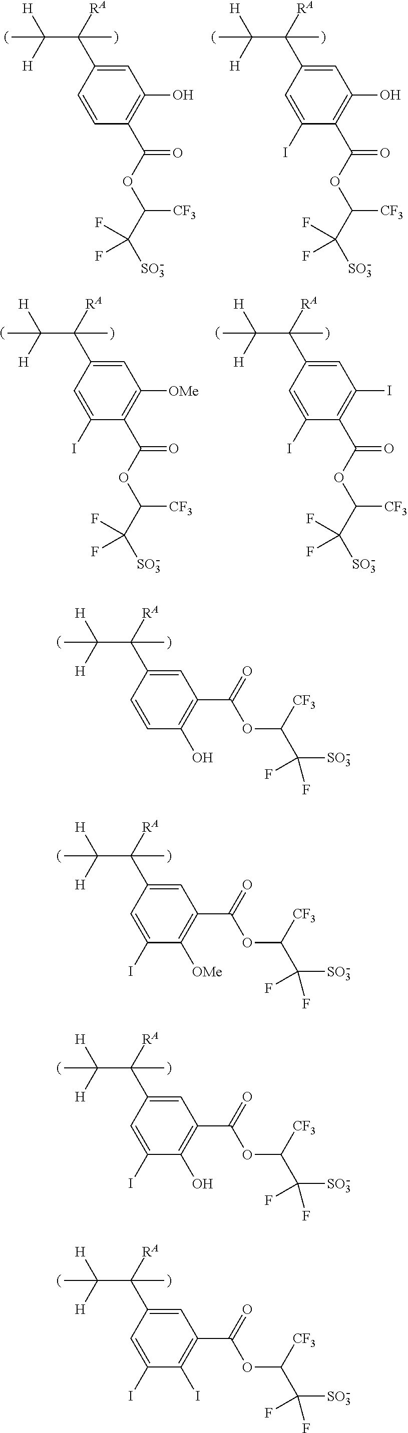

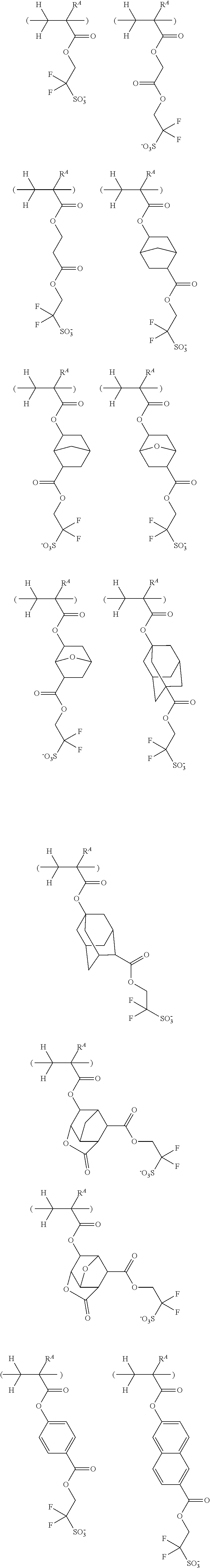

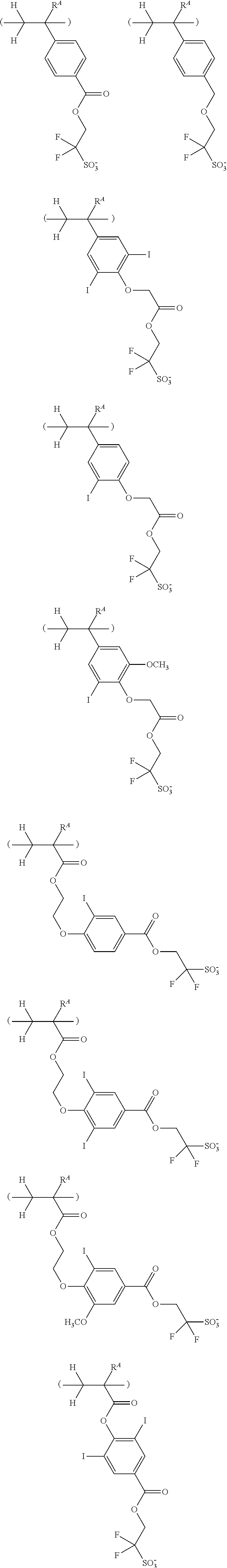

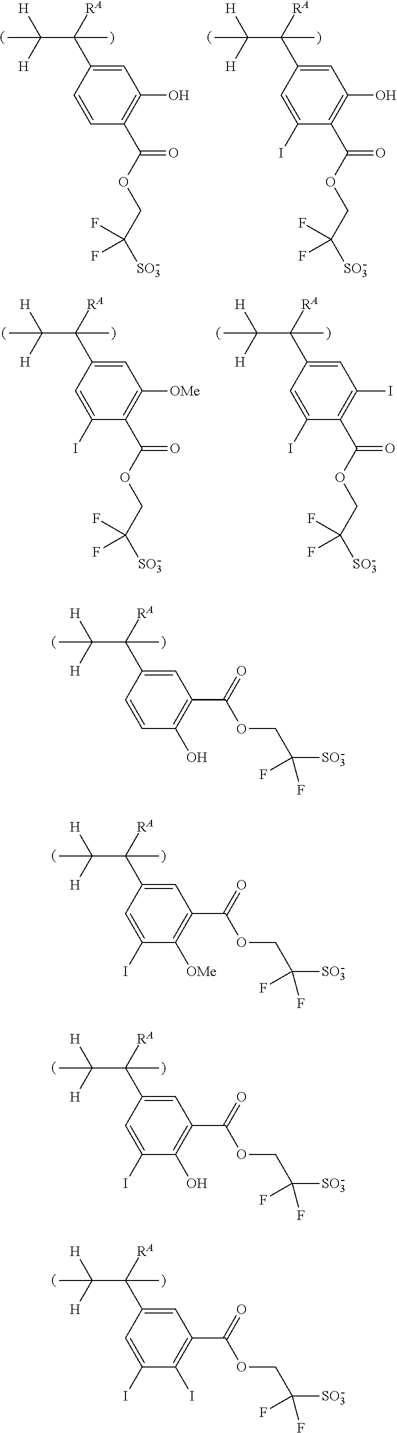

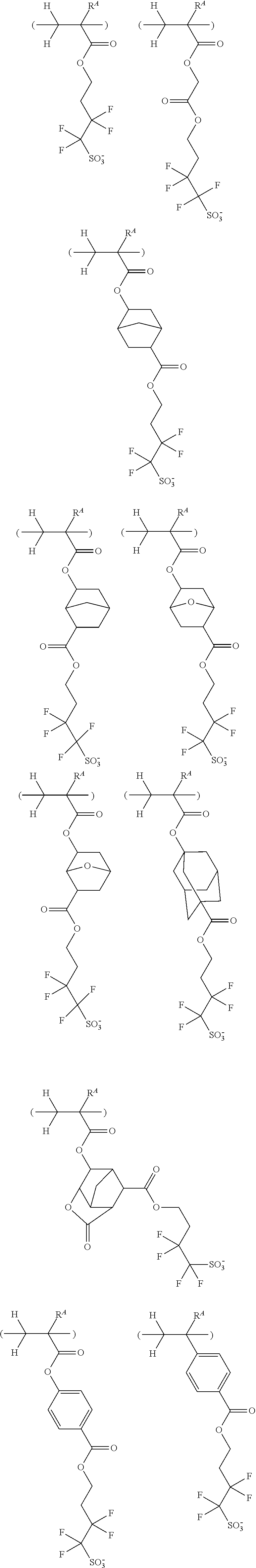

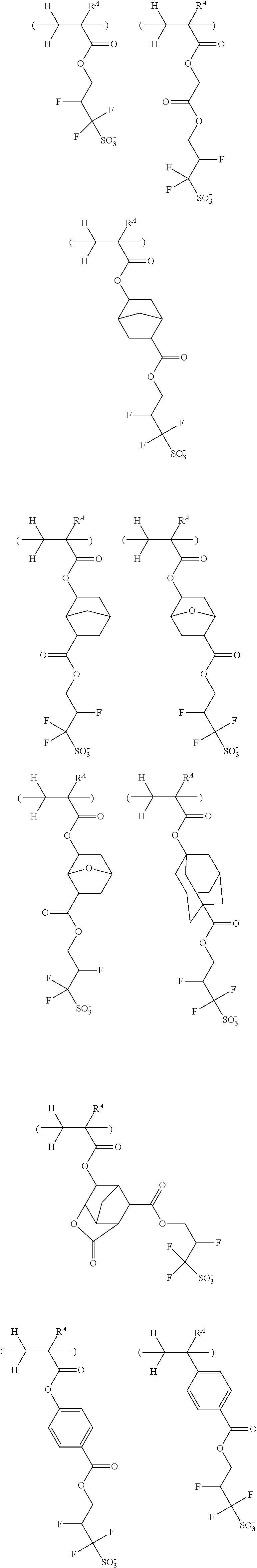

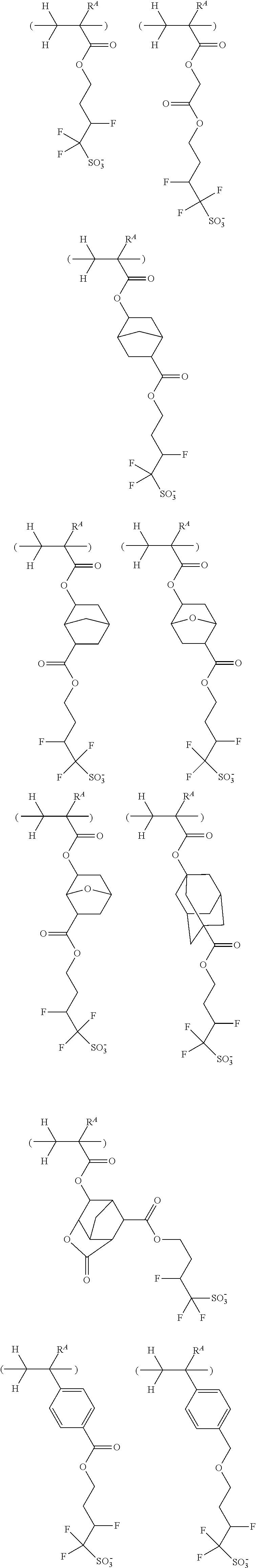

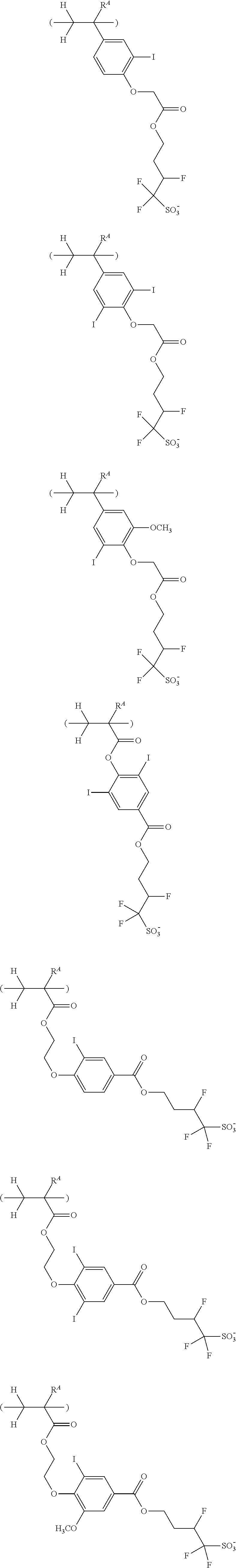

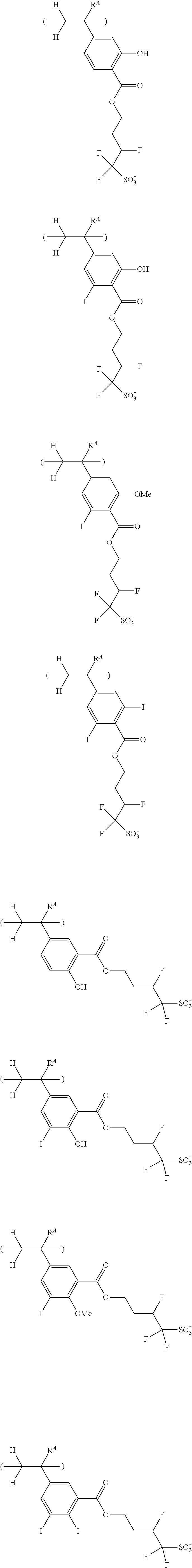

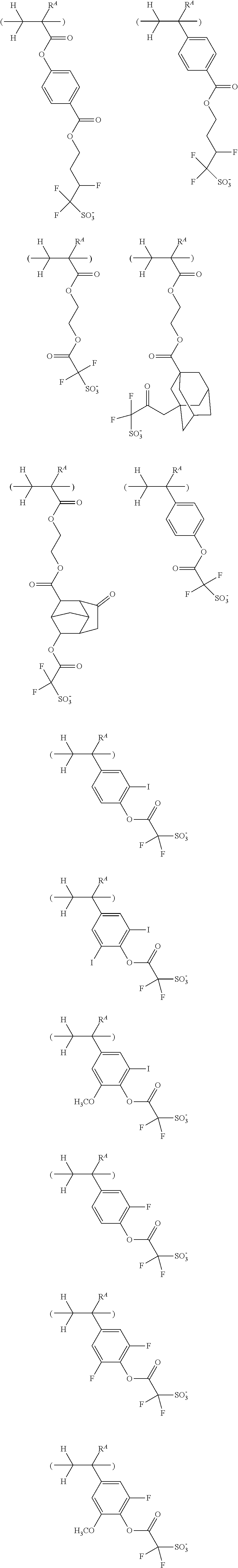

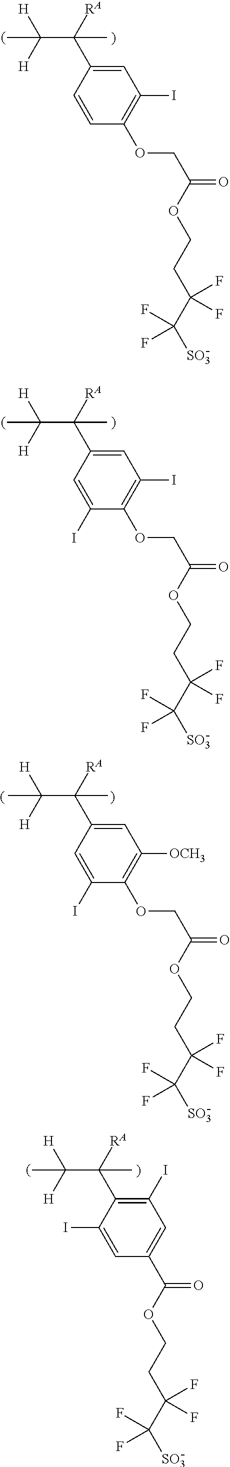

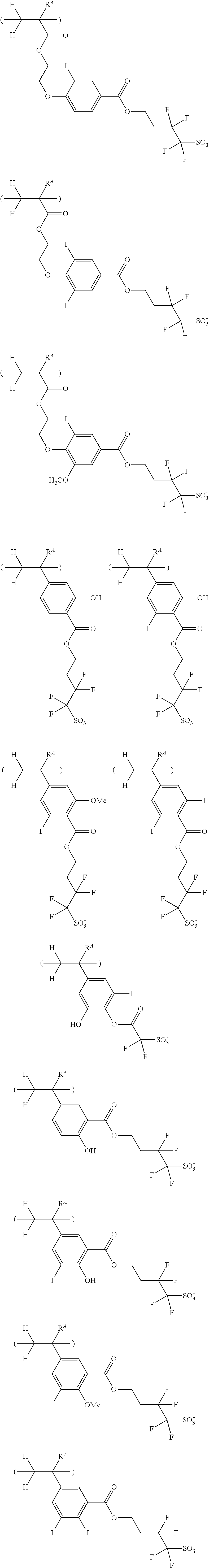

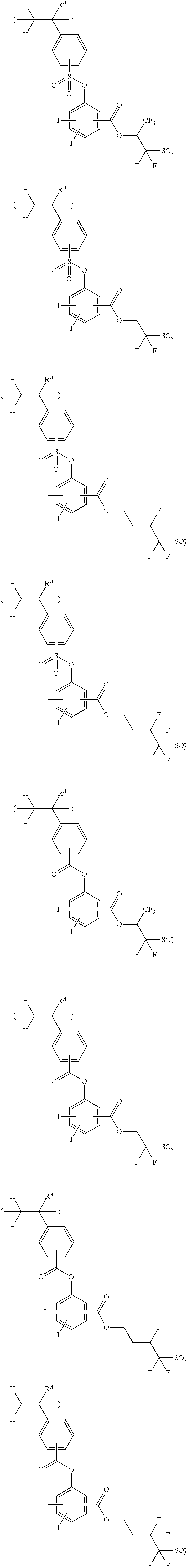

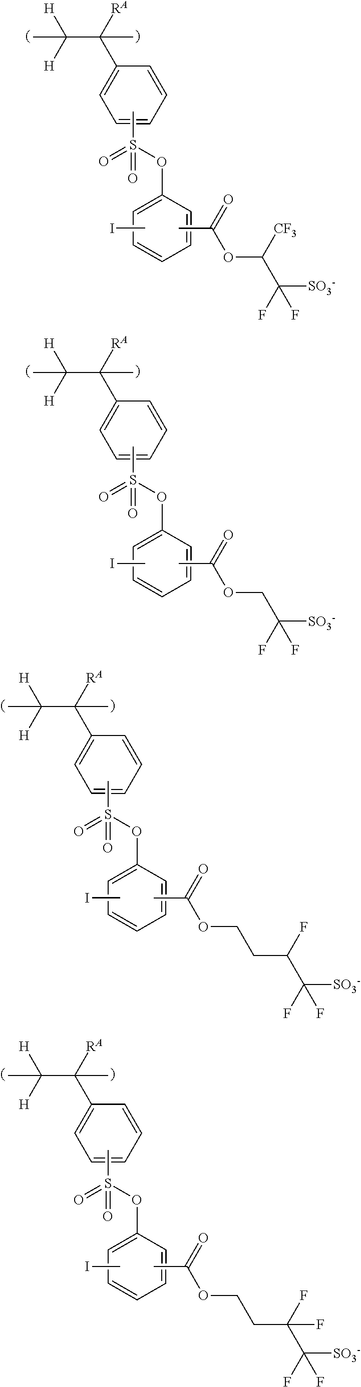

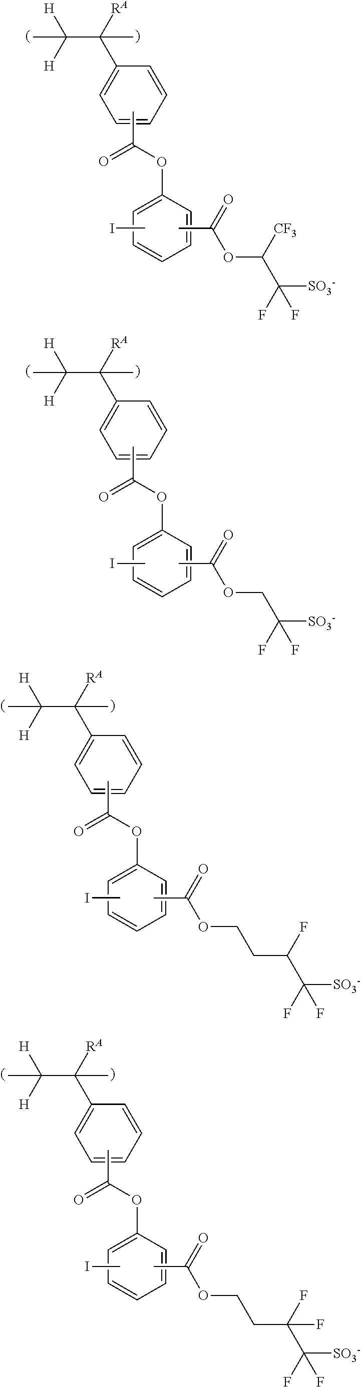

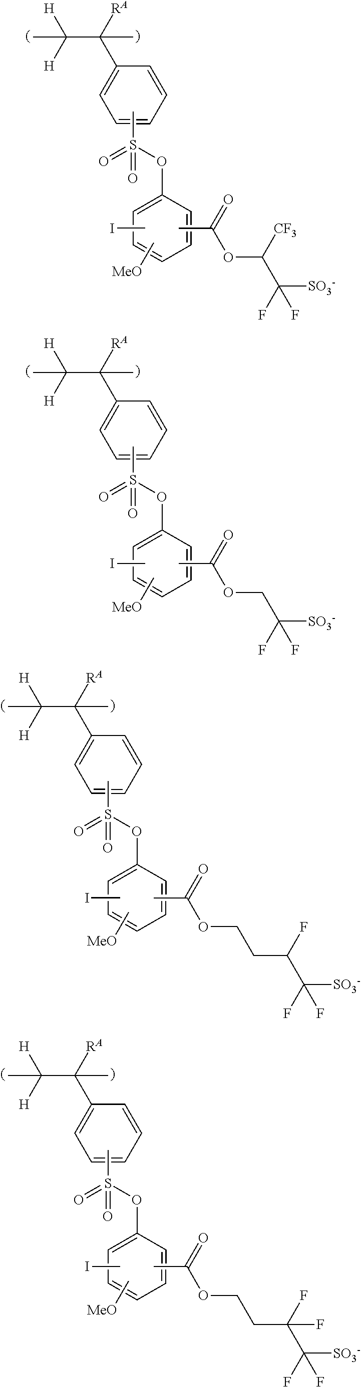

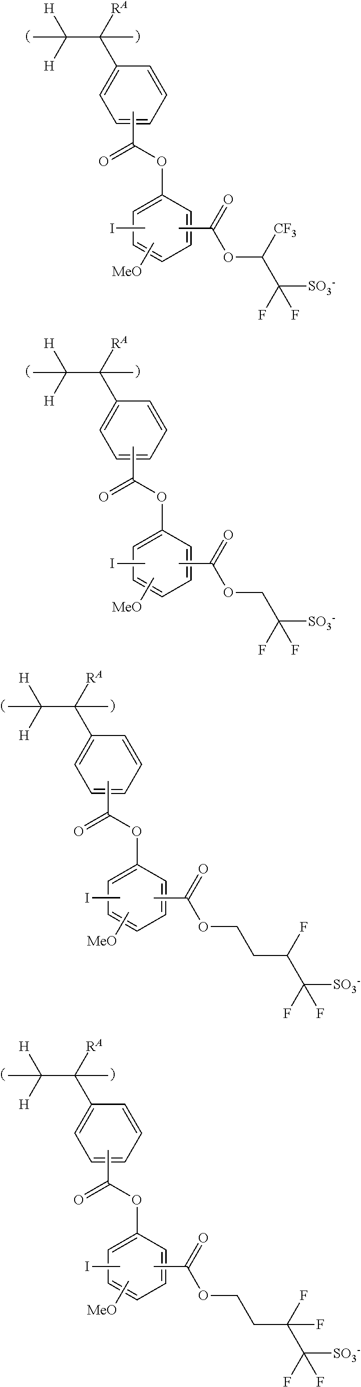

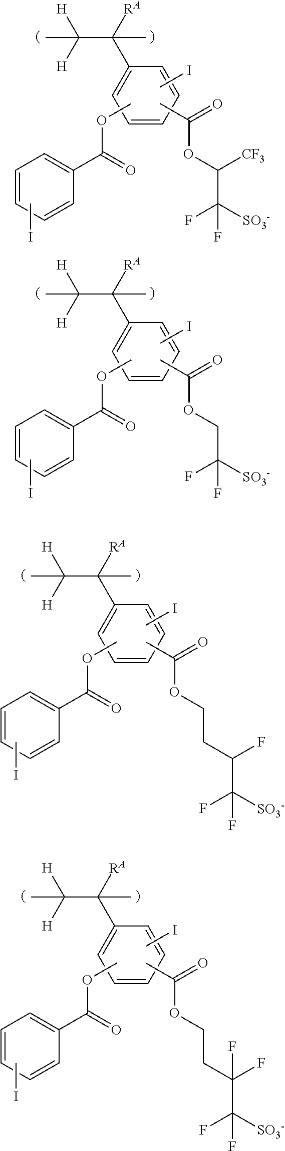

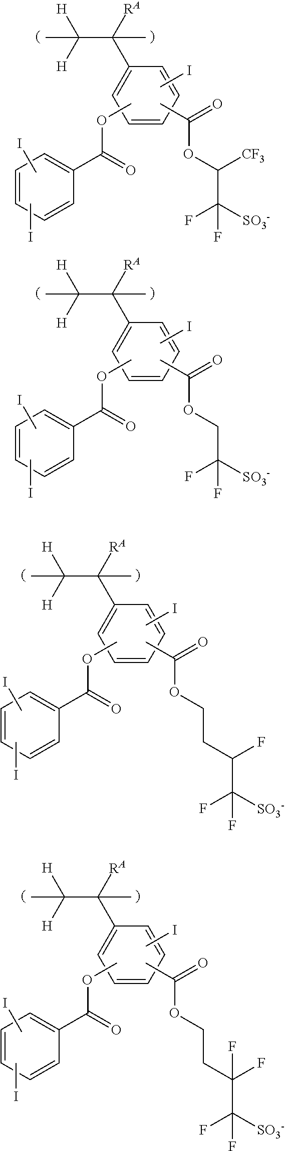

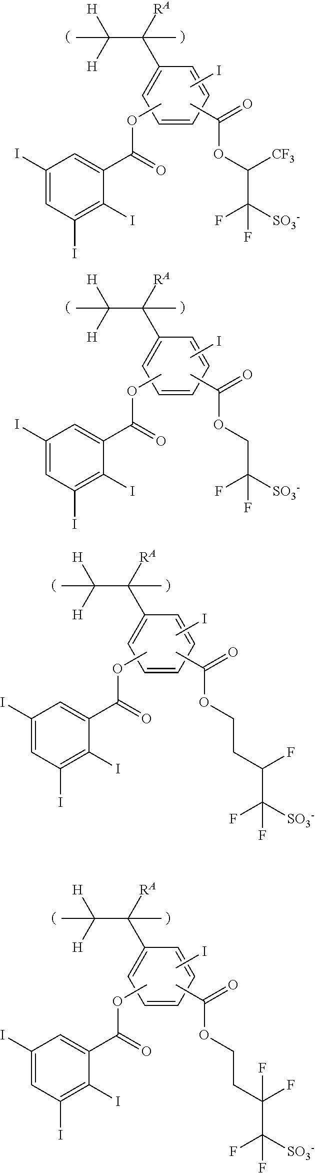

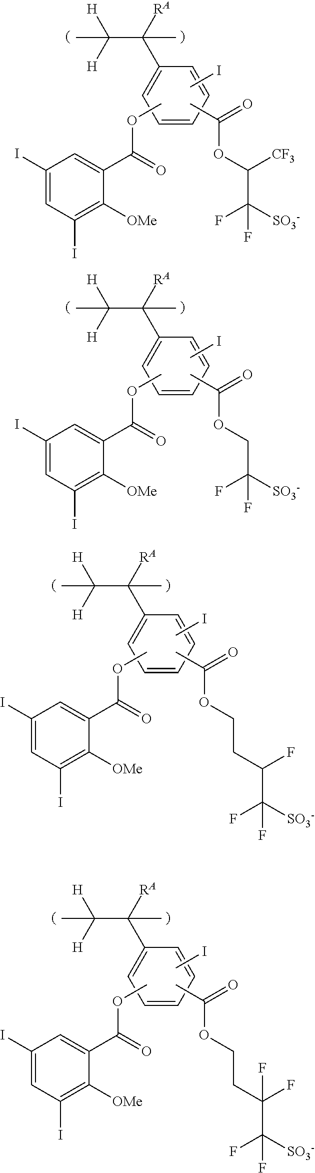

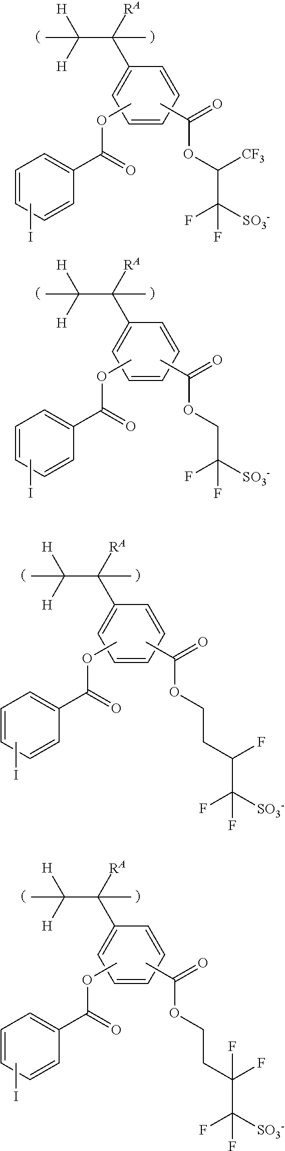

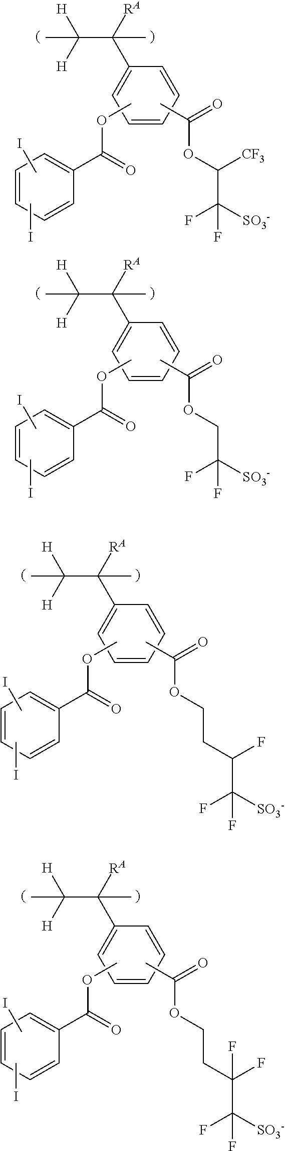

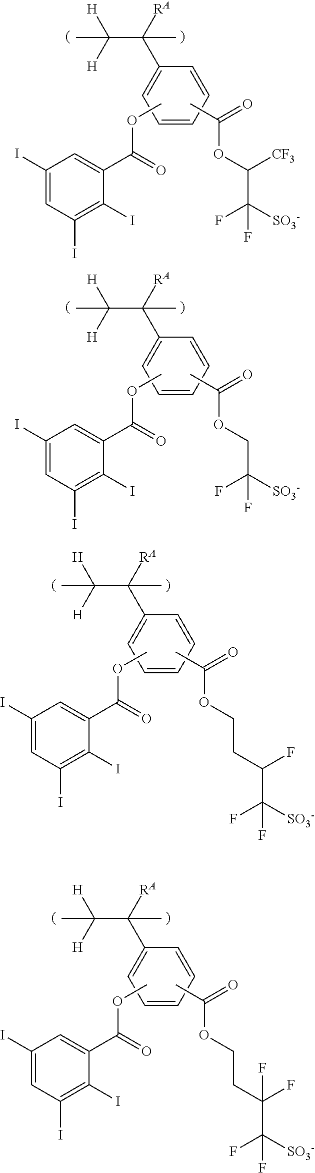

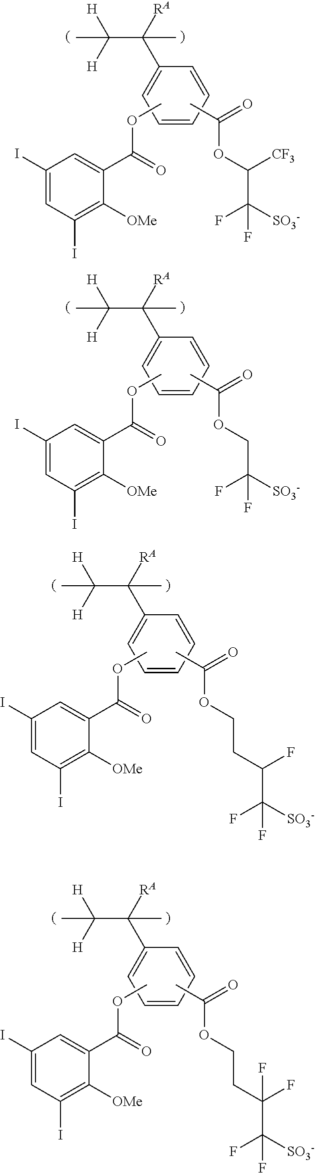

Specific examples of the anion of the repeating unit-c3 include, but are not limited to, the following. Note that in the following formulae, R^{A} is as defined above, and Me represents a methyl group.

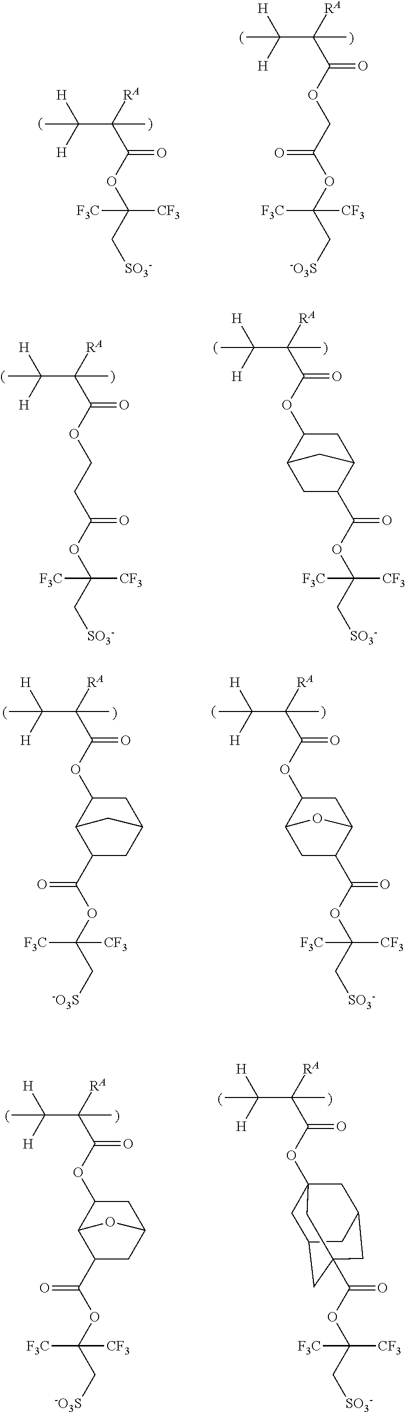

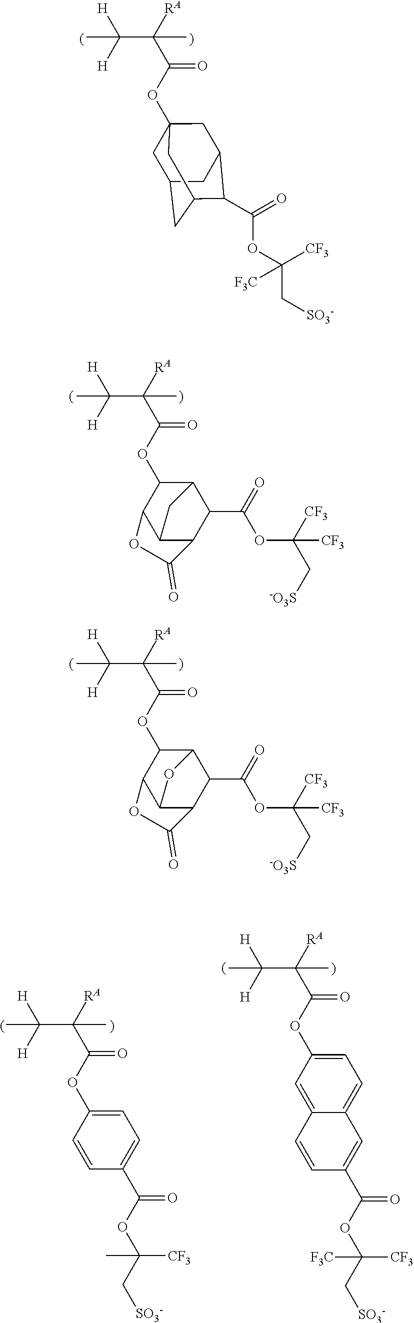

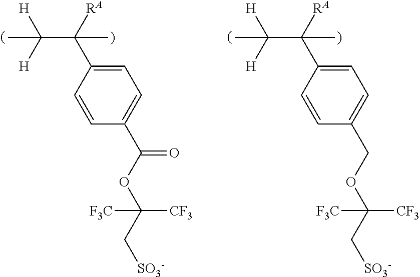

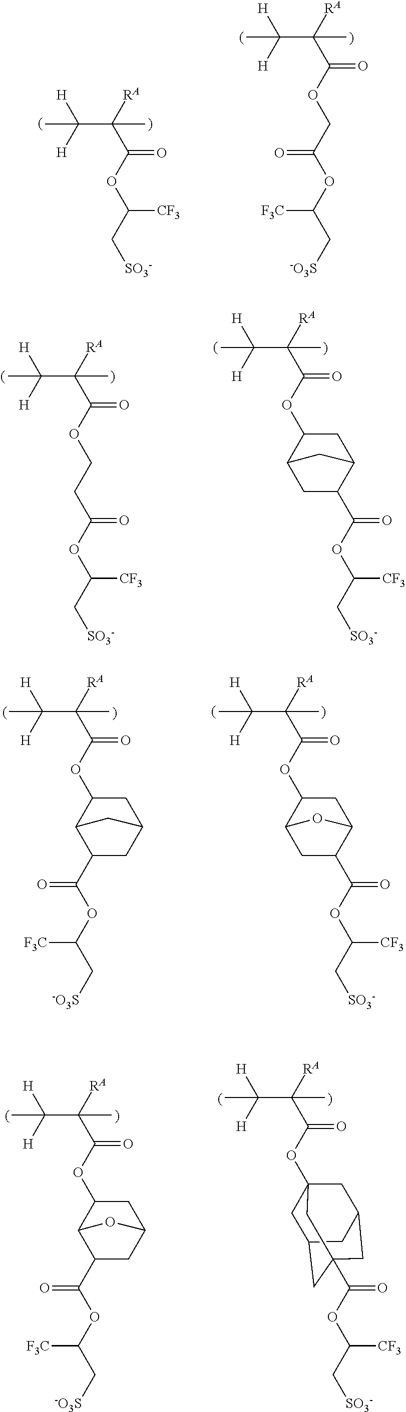

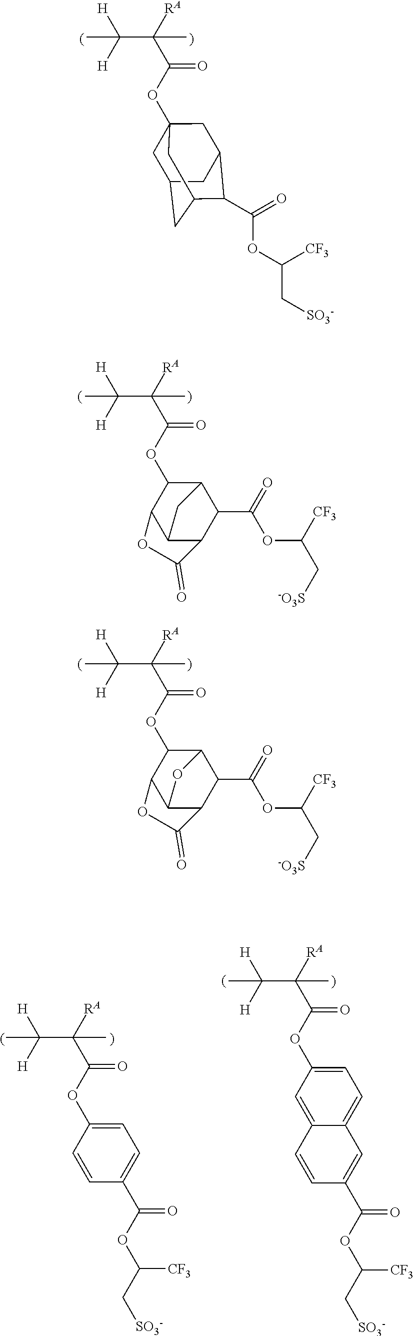

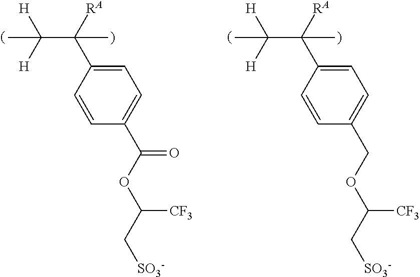

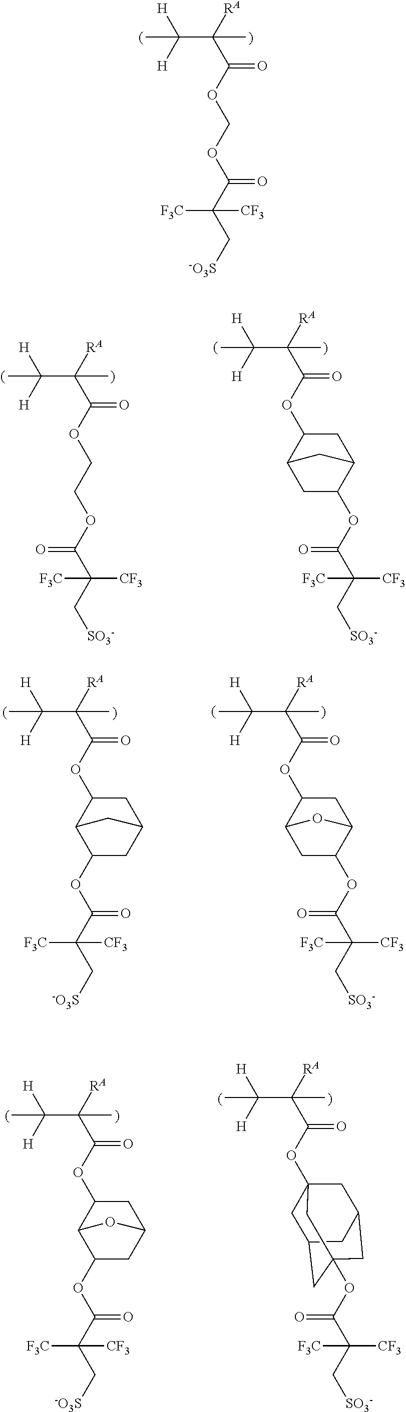

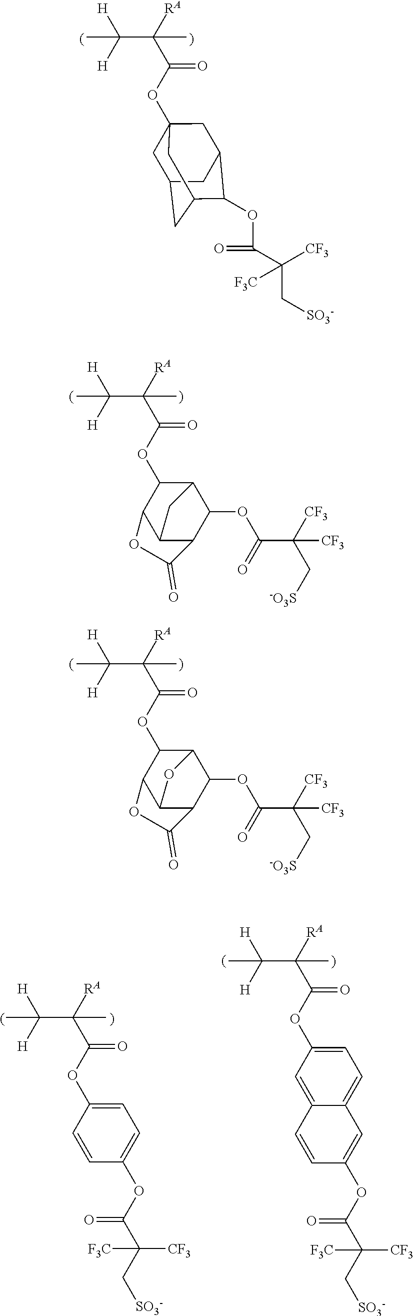

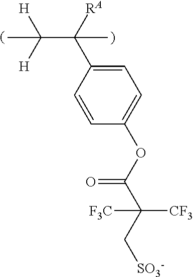

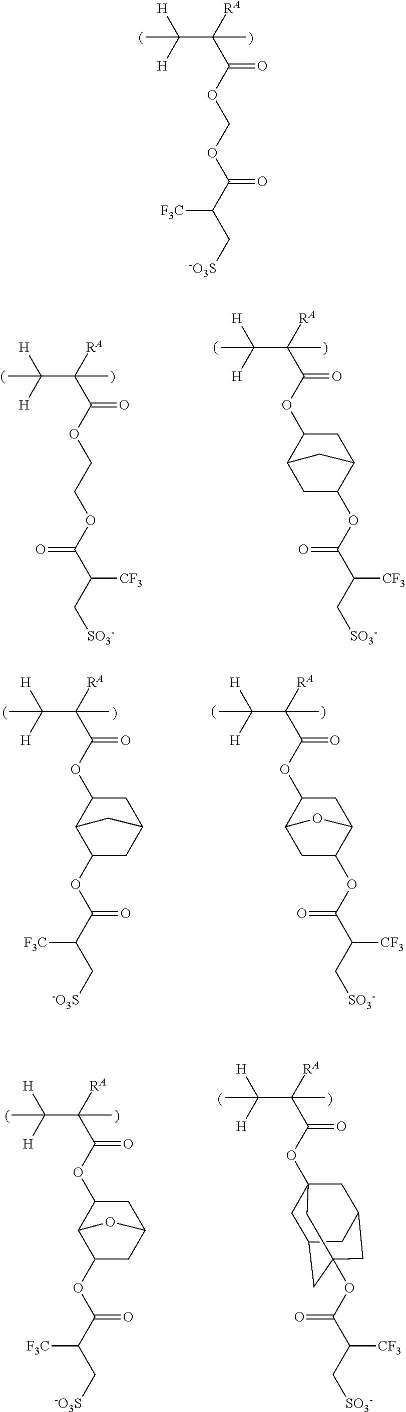

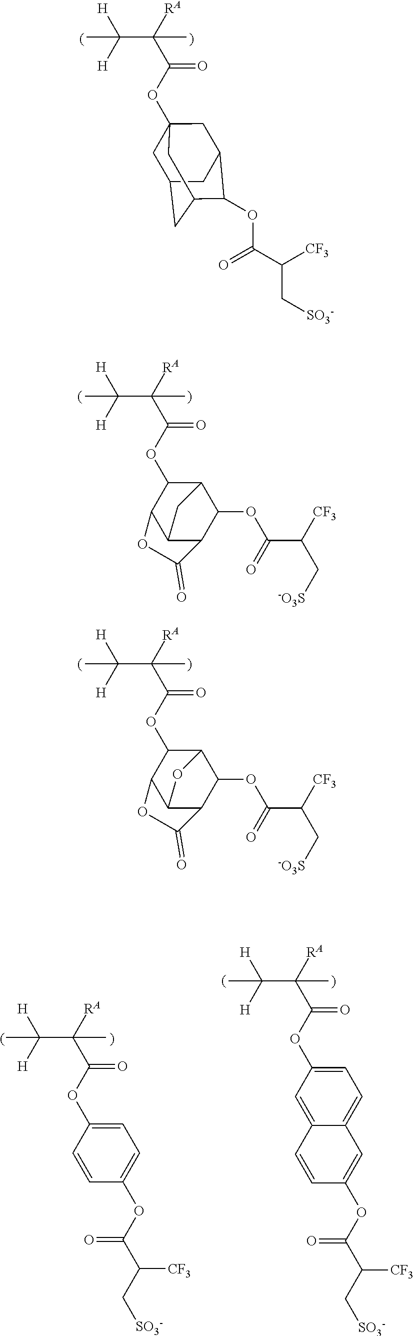

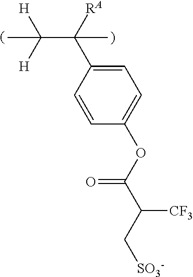

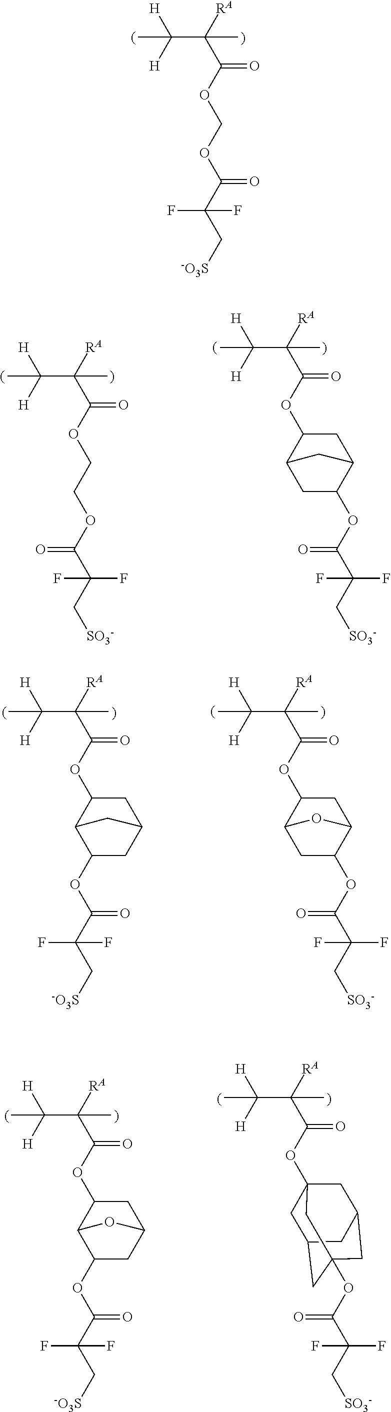

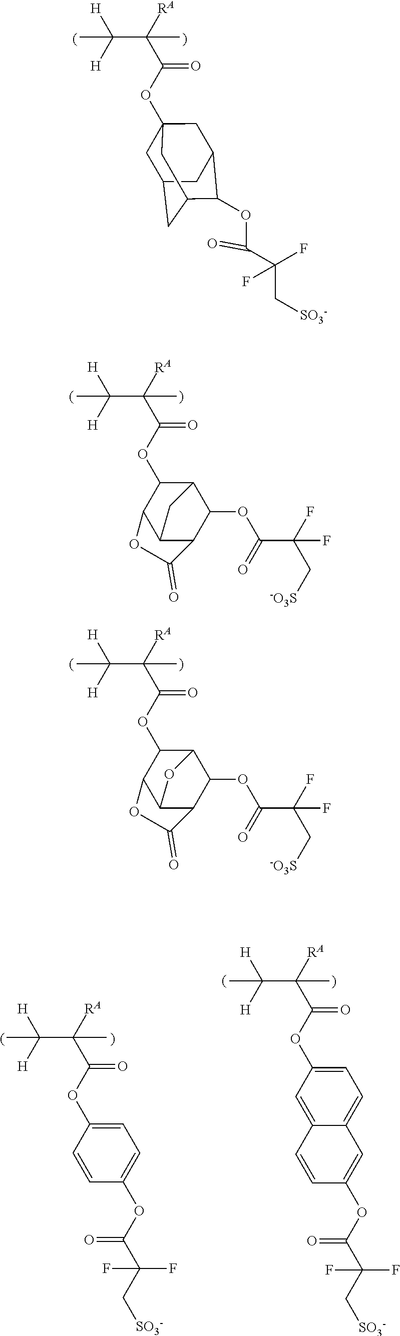

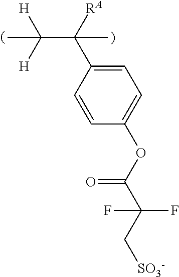

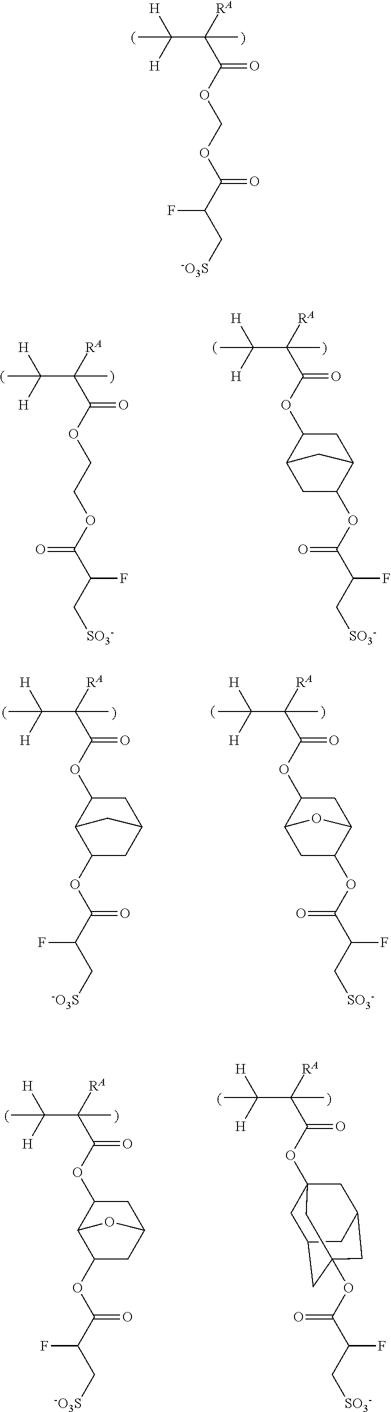

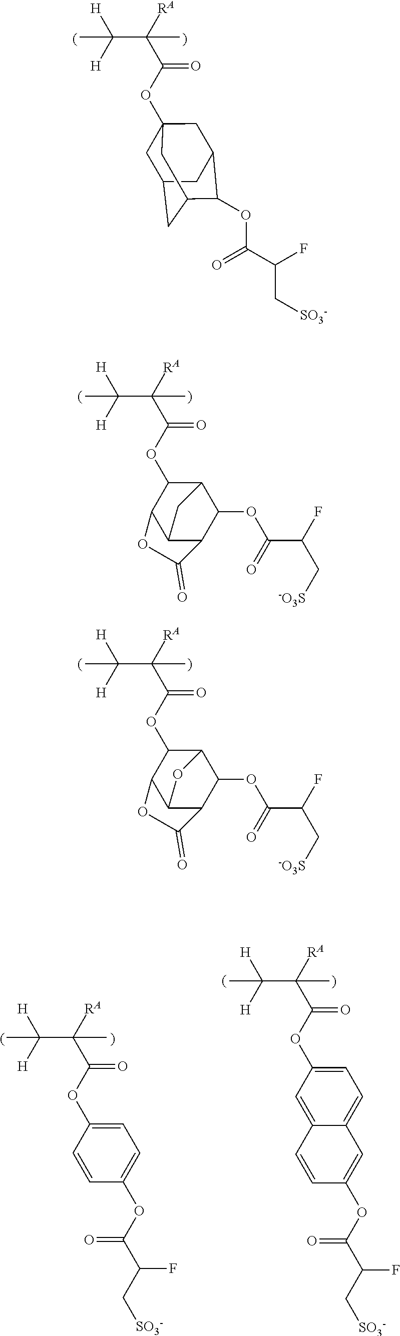

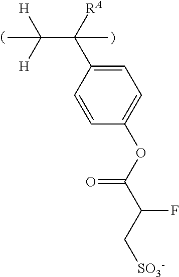

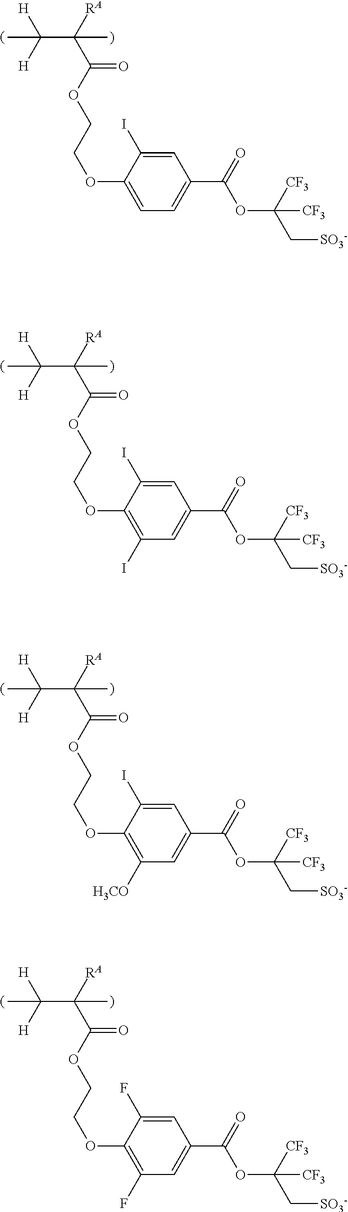

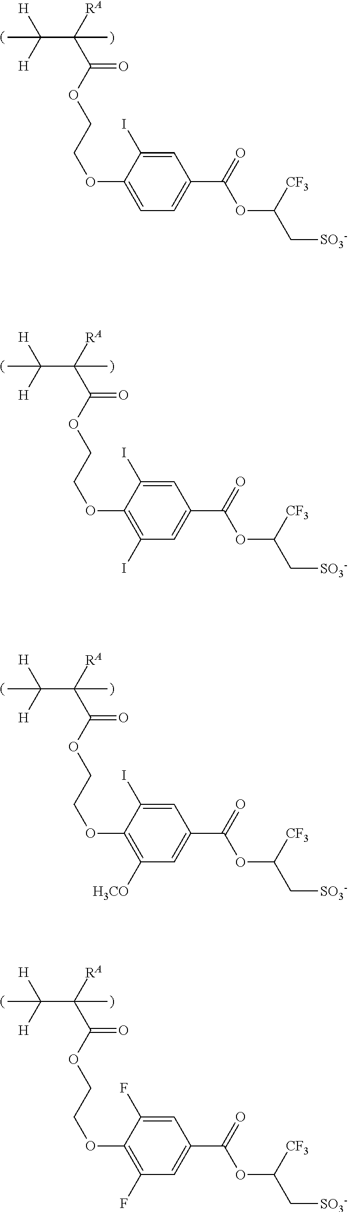

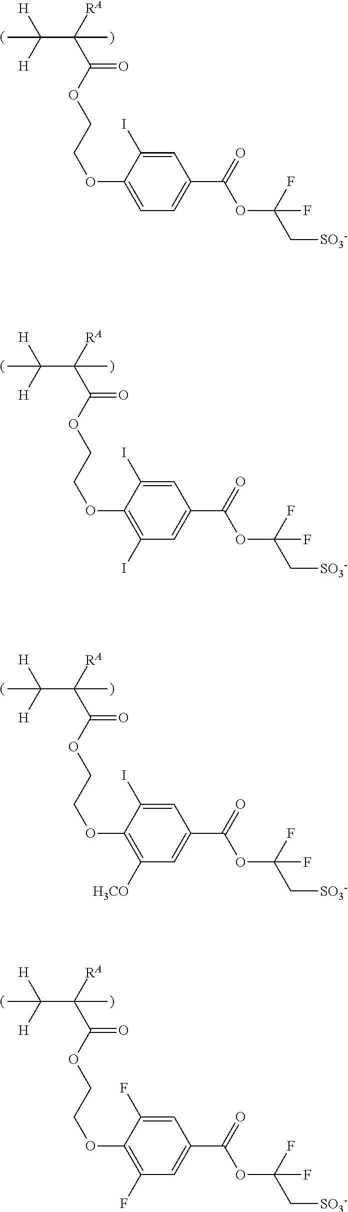

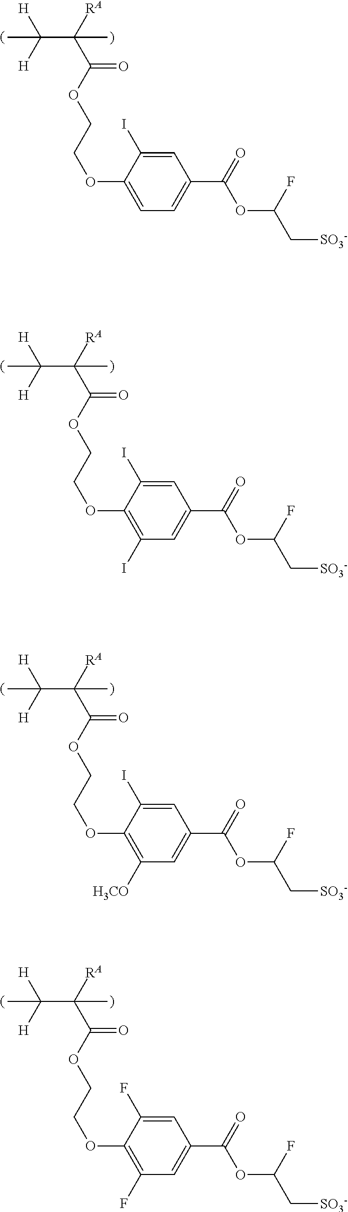

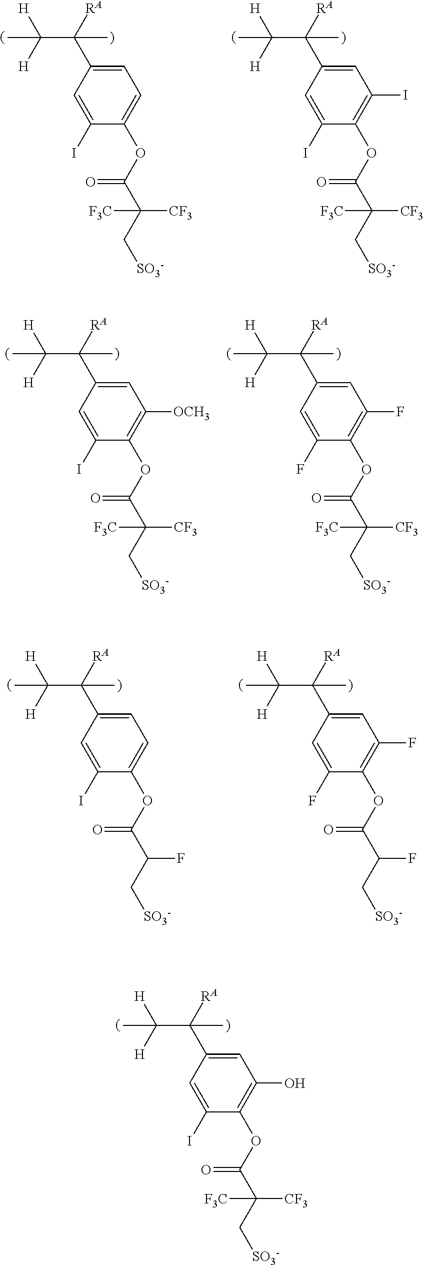

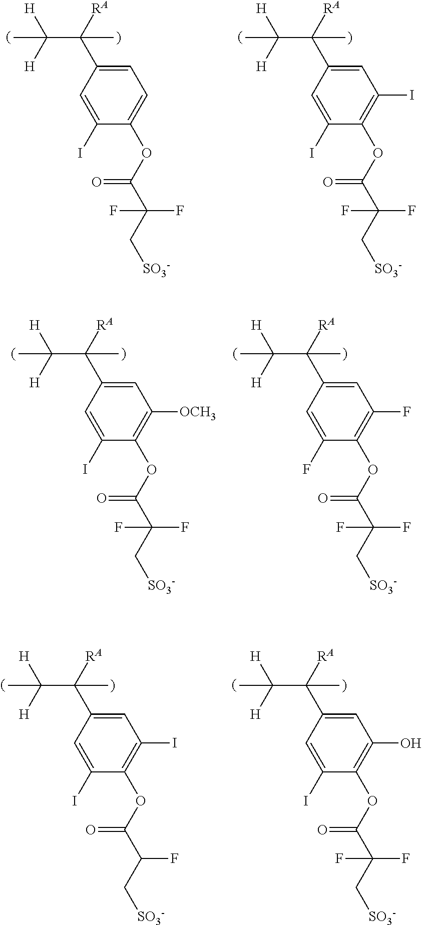

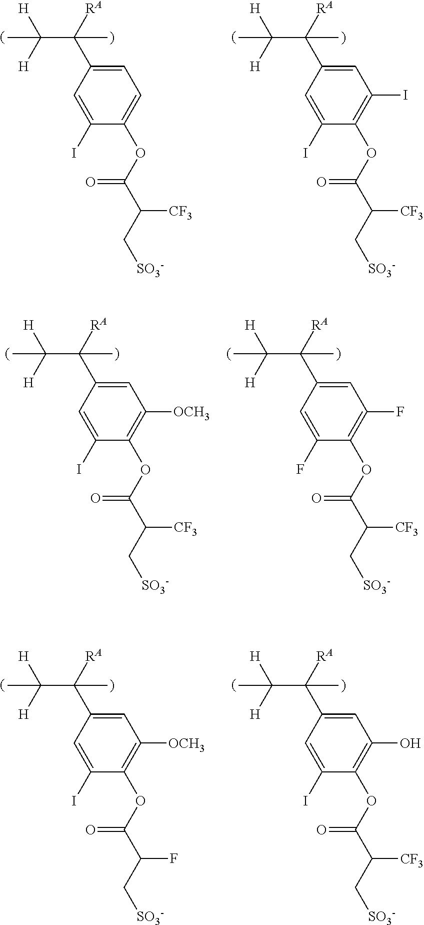

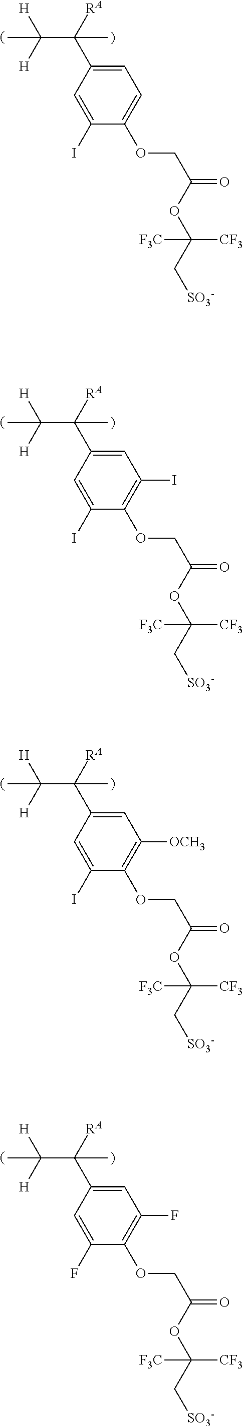

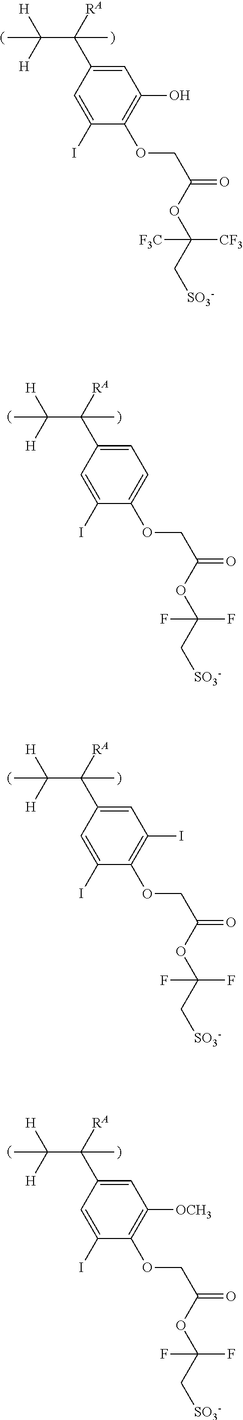

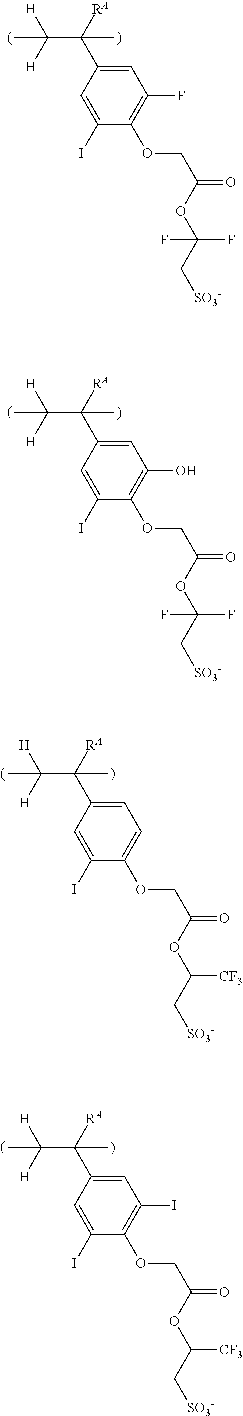

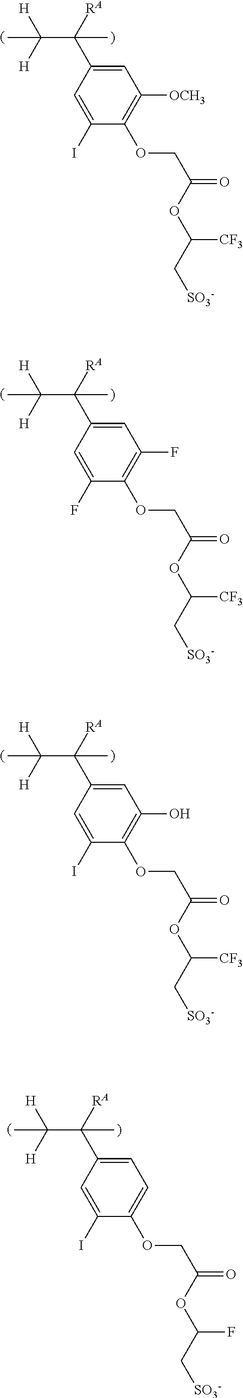

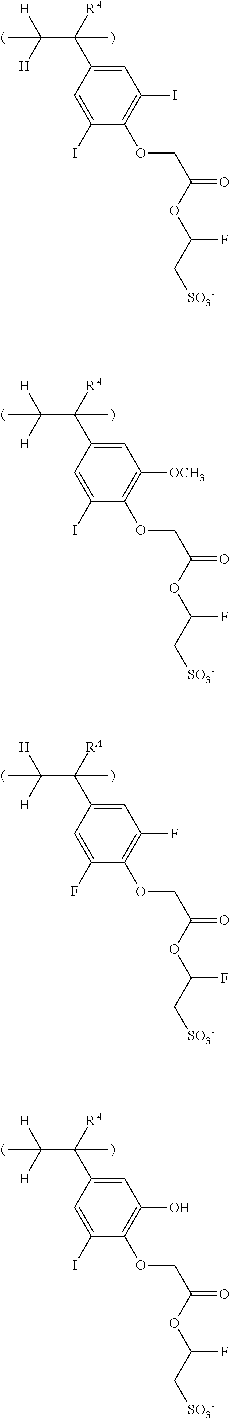

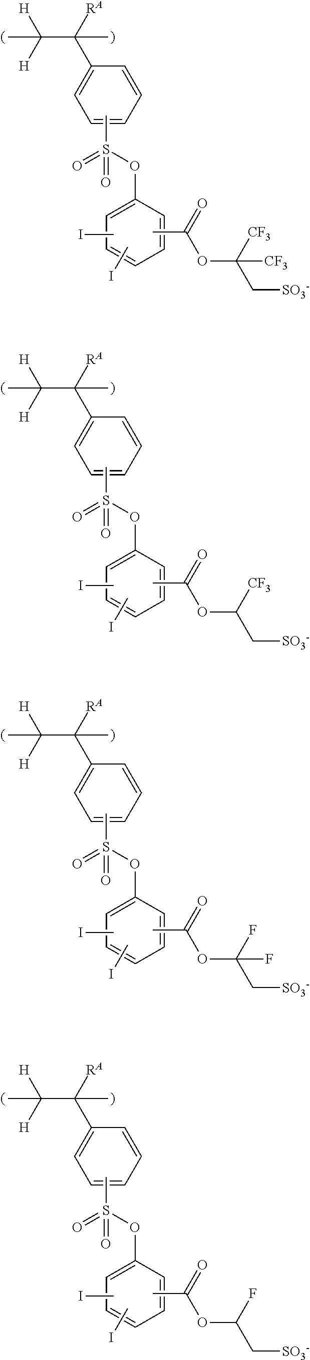

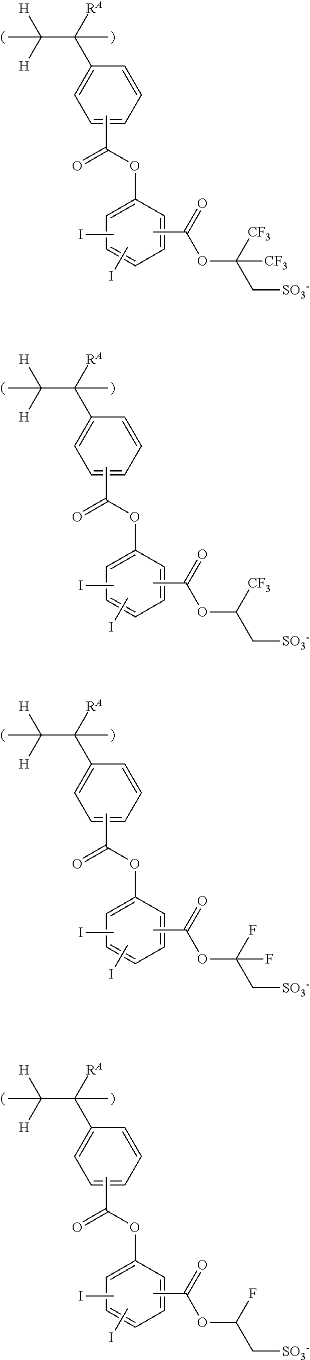

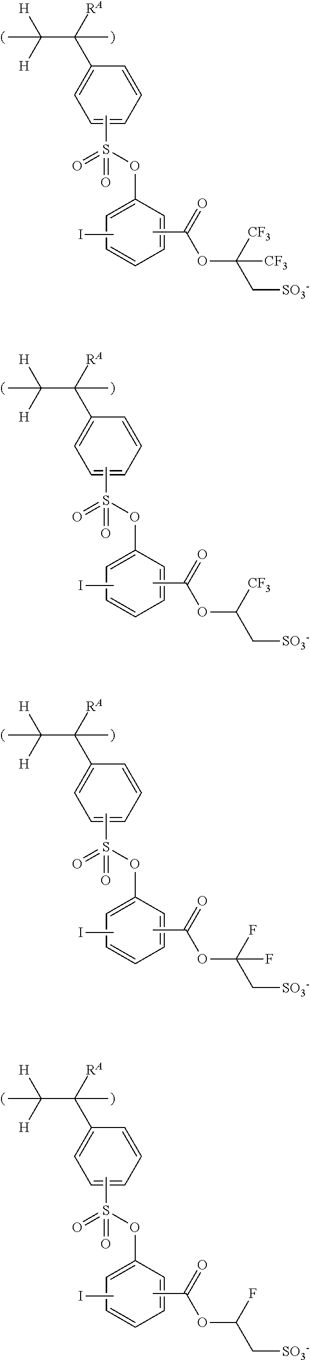

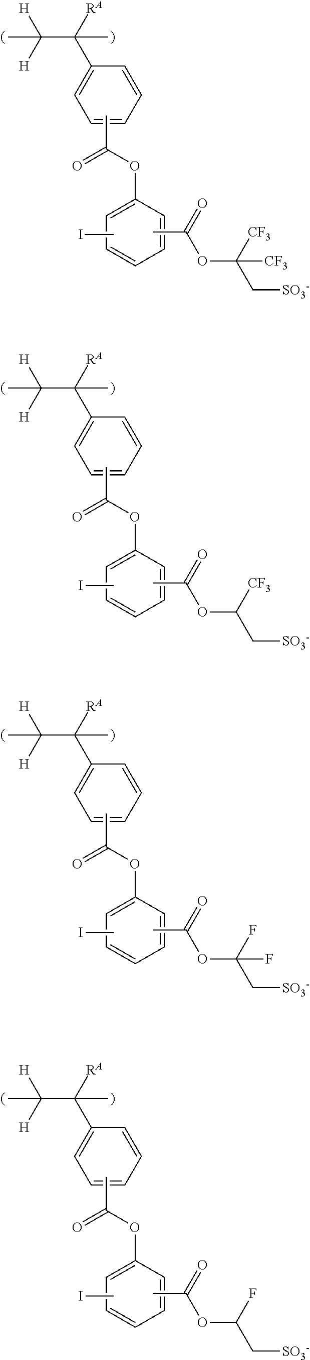

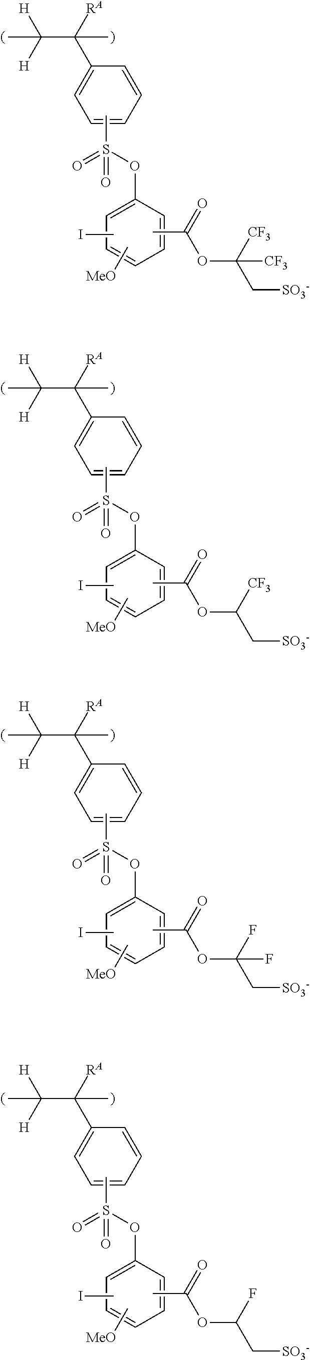

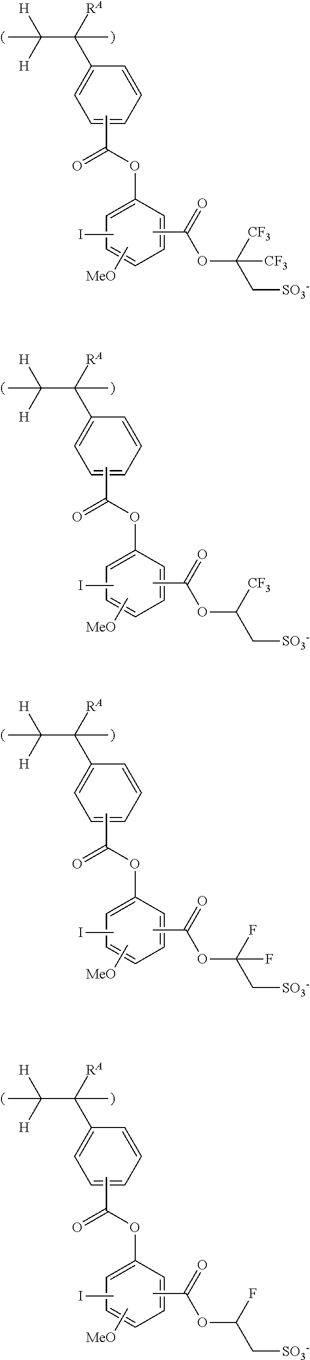

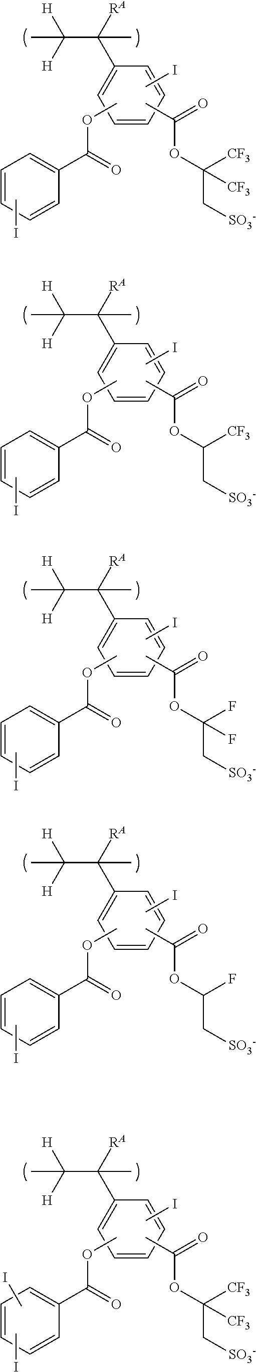

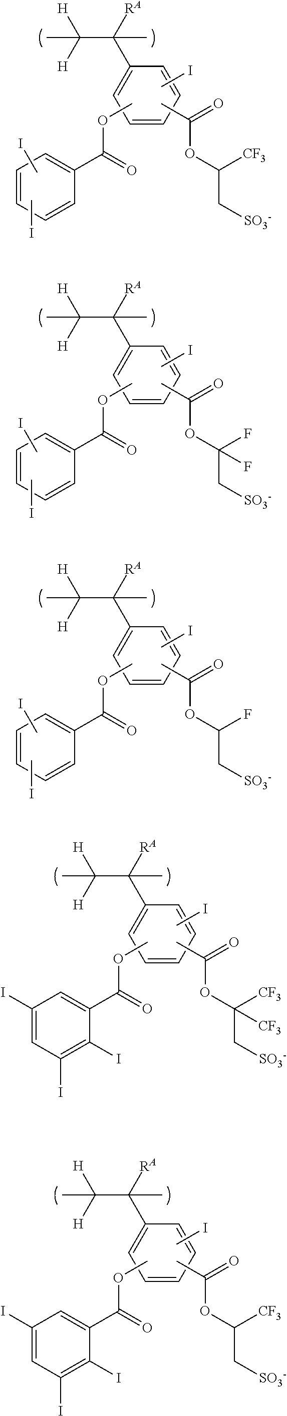

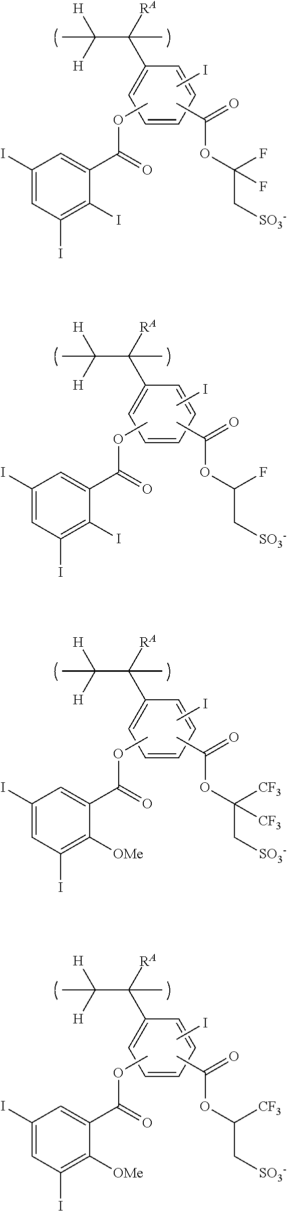

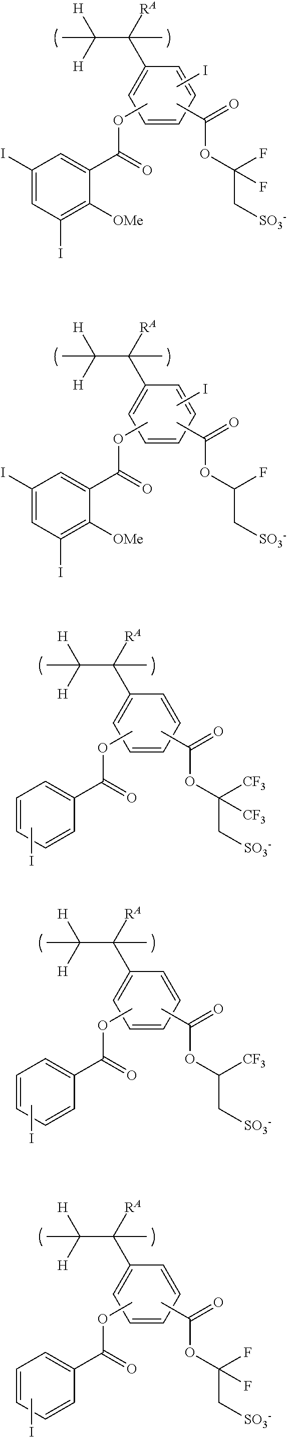

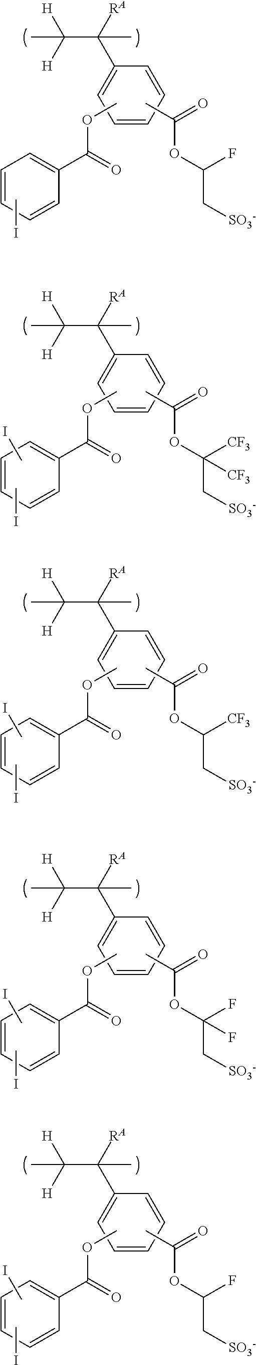

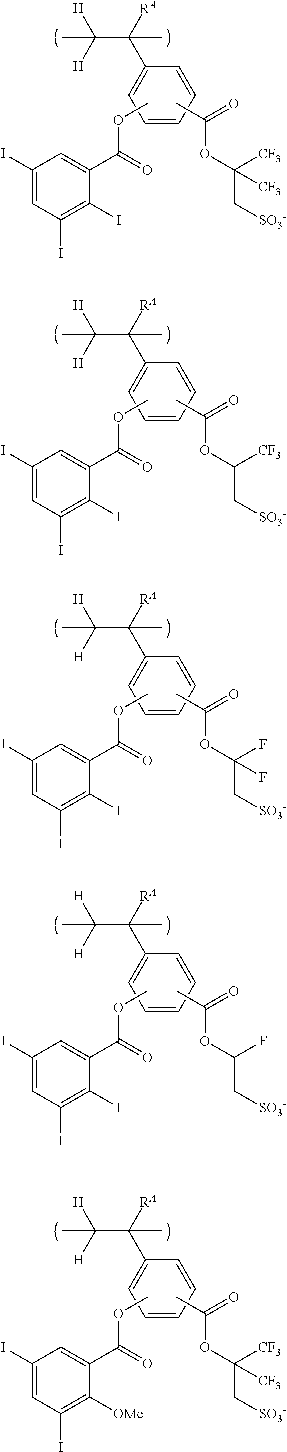

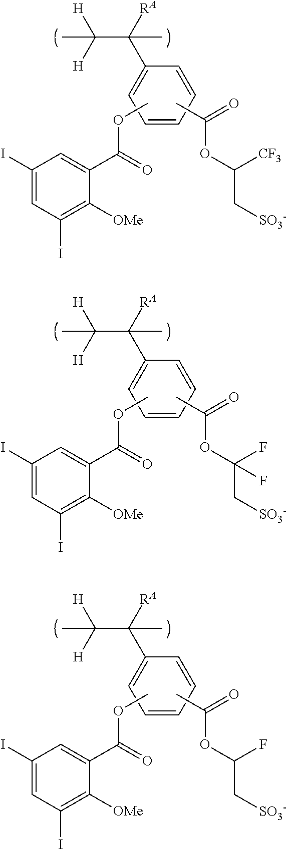

Specific examples of the anion of the repeating unit-c4 include, but are not limited to, the following. Note that in the following formulae, R^{A} is as defined above, and Me represents a methyl group.

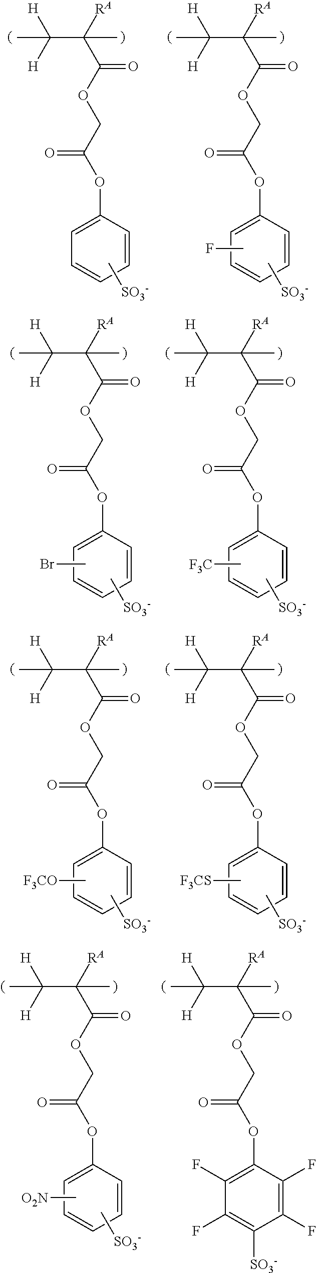

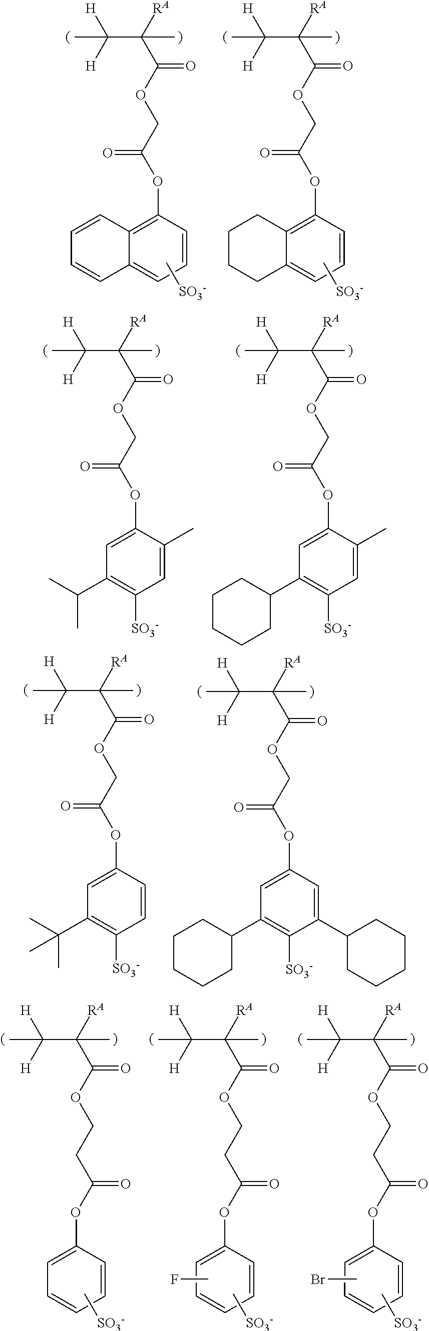

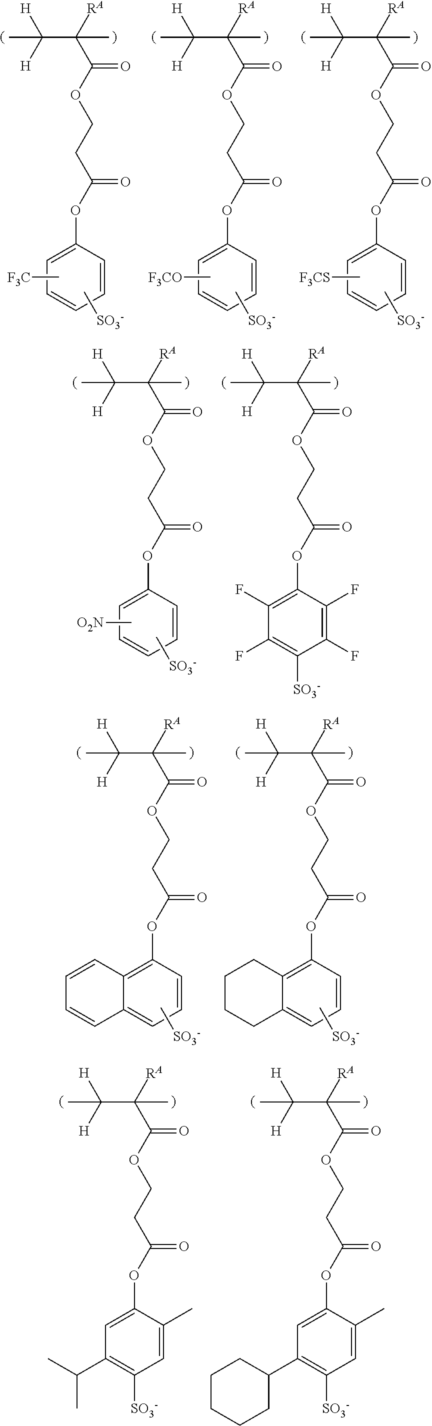

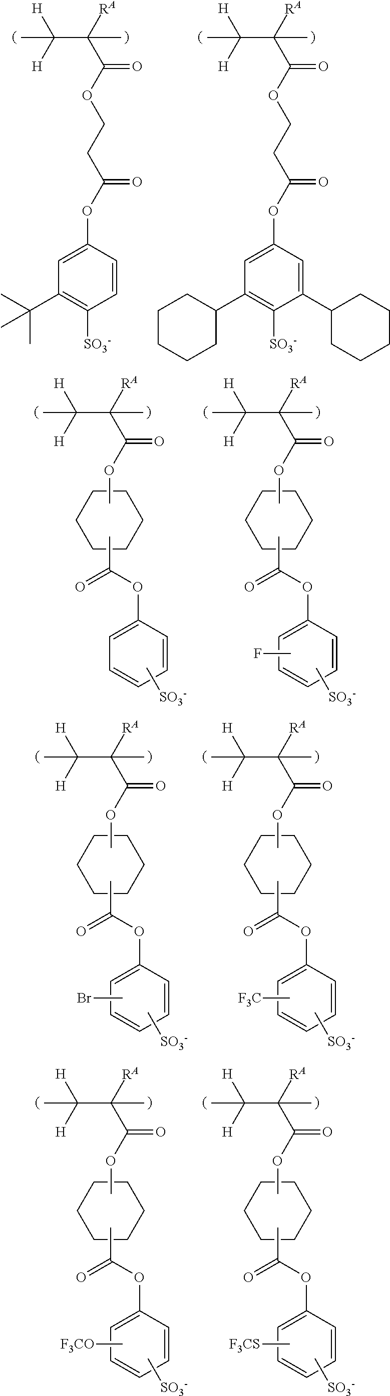

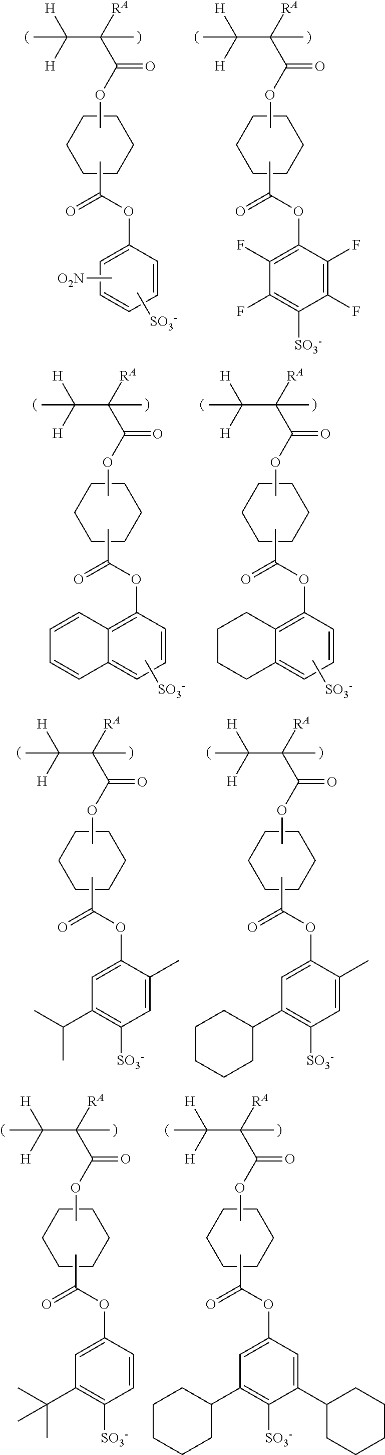

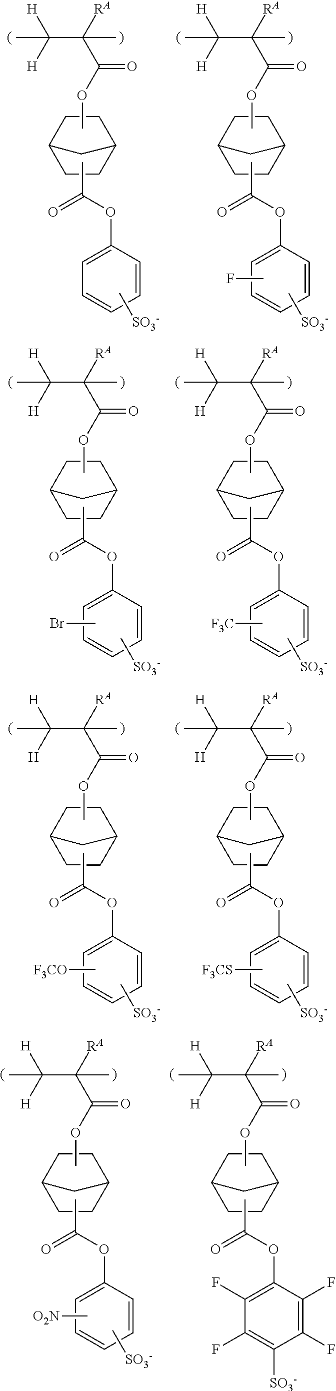

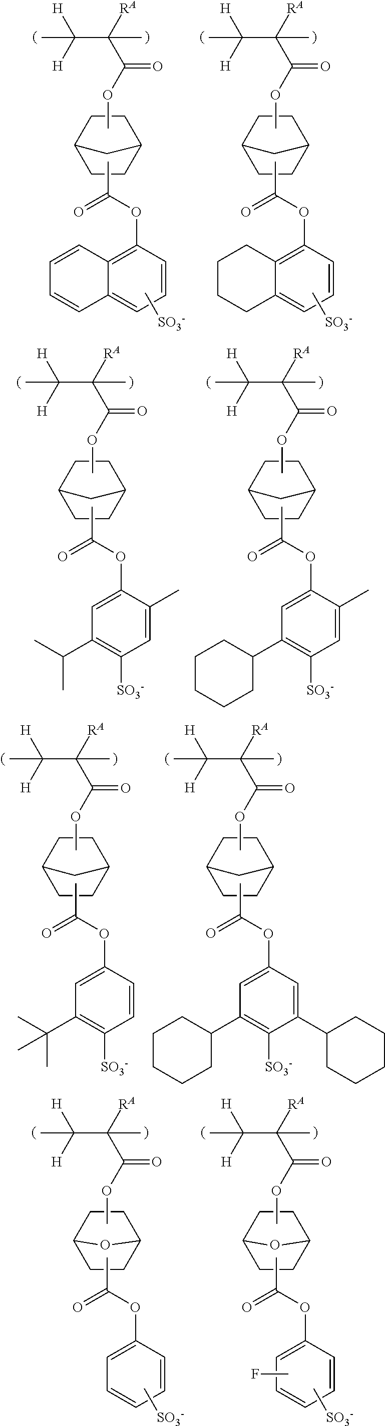

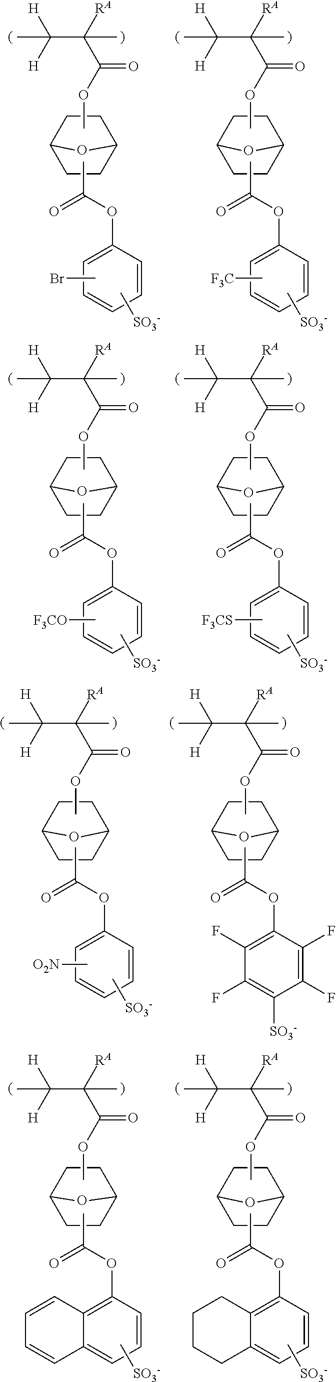

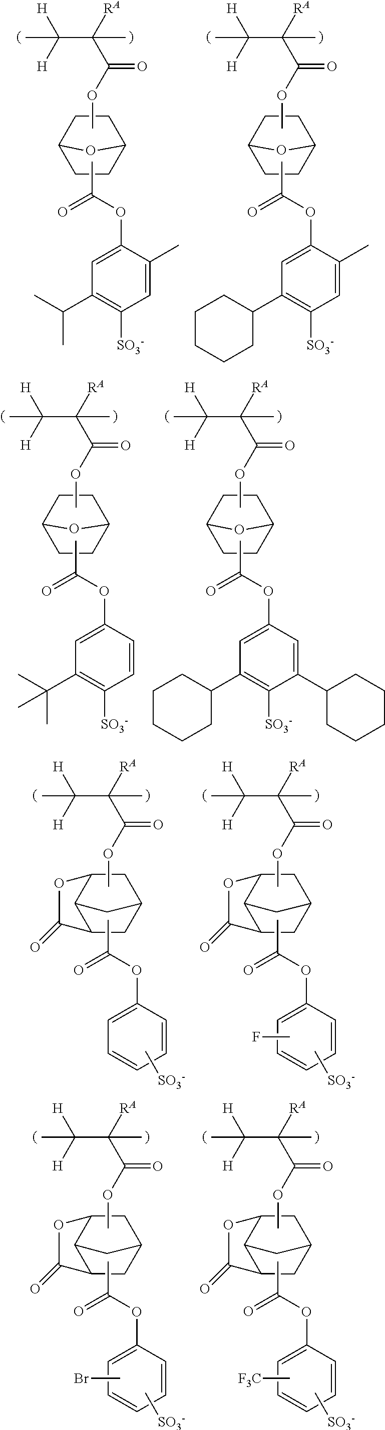

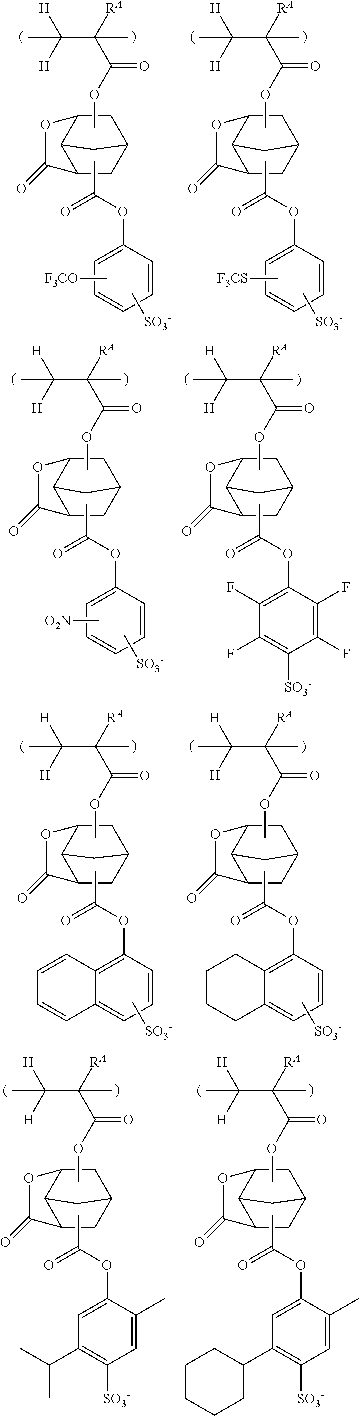

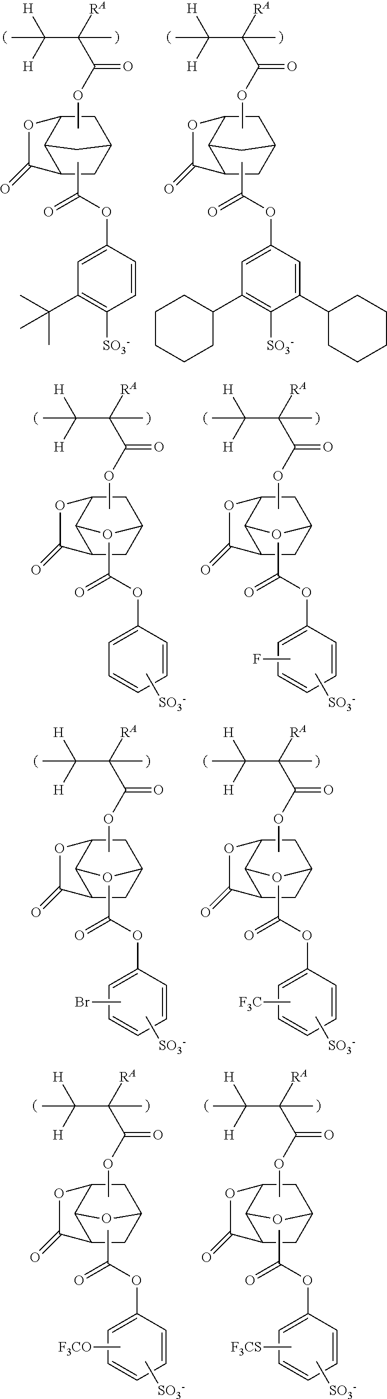

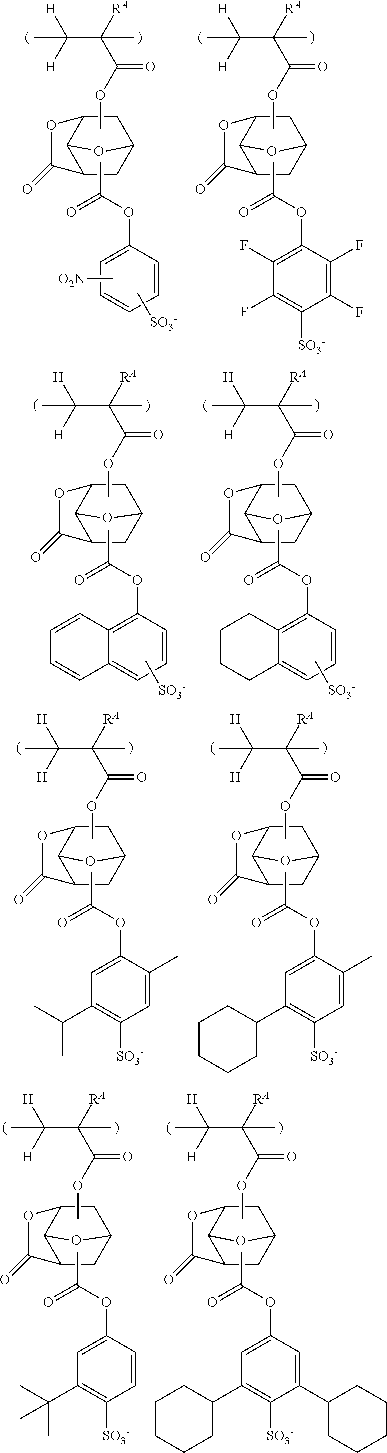

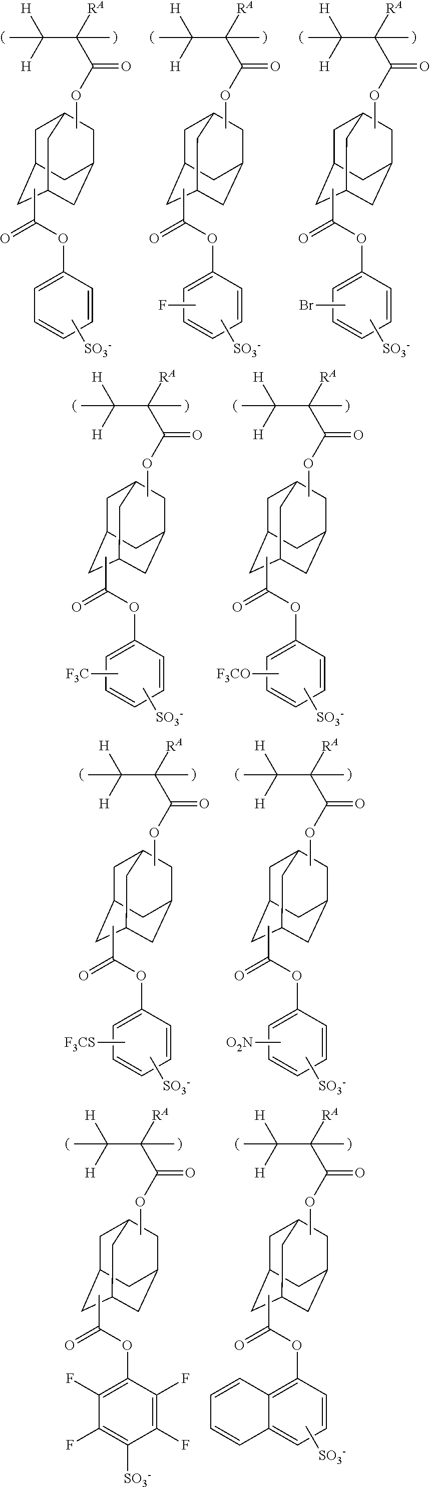

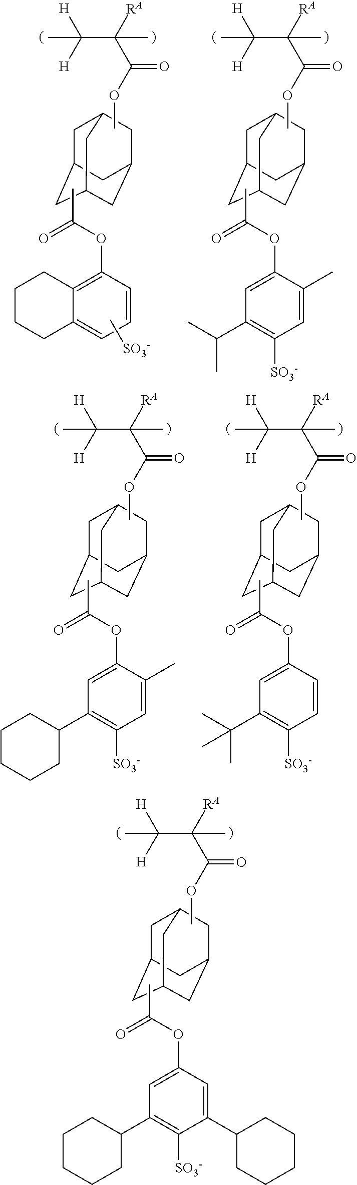

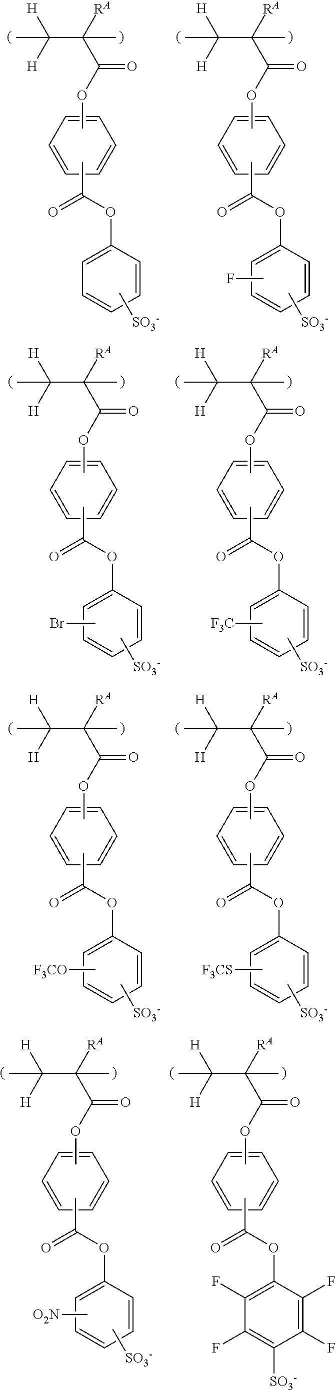

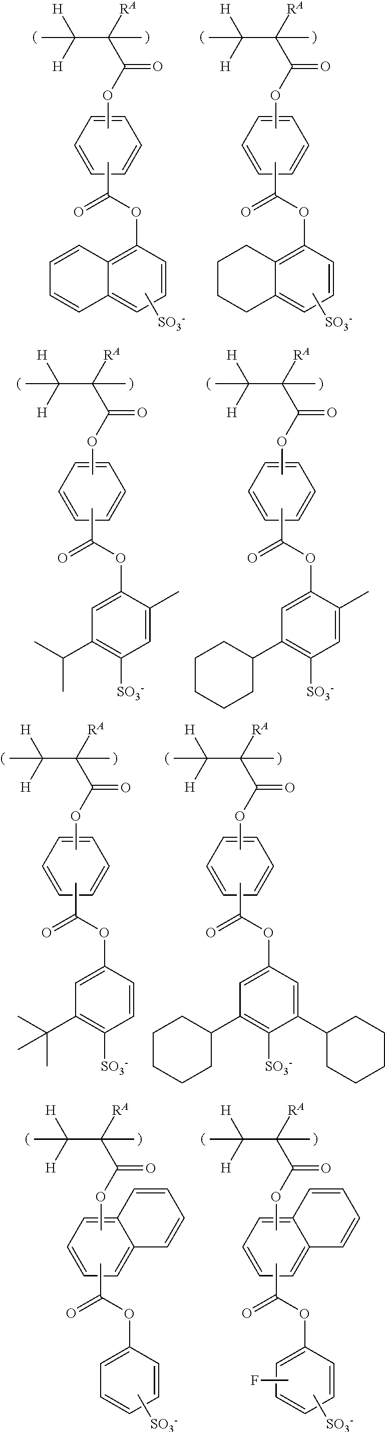

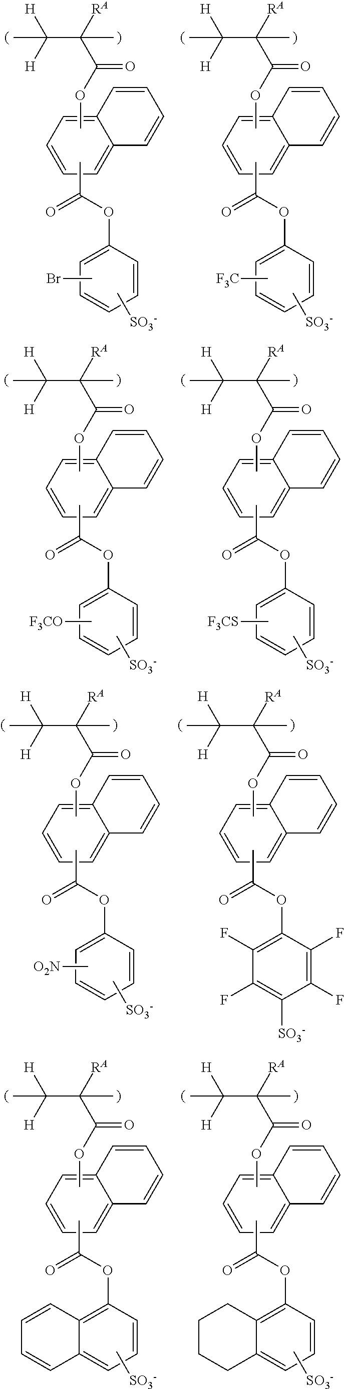

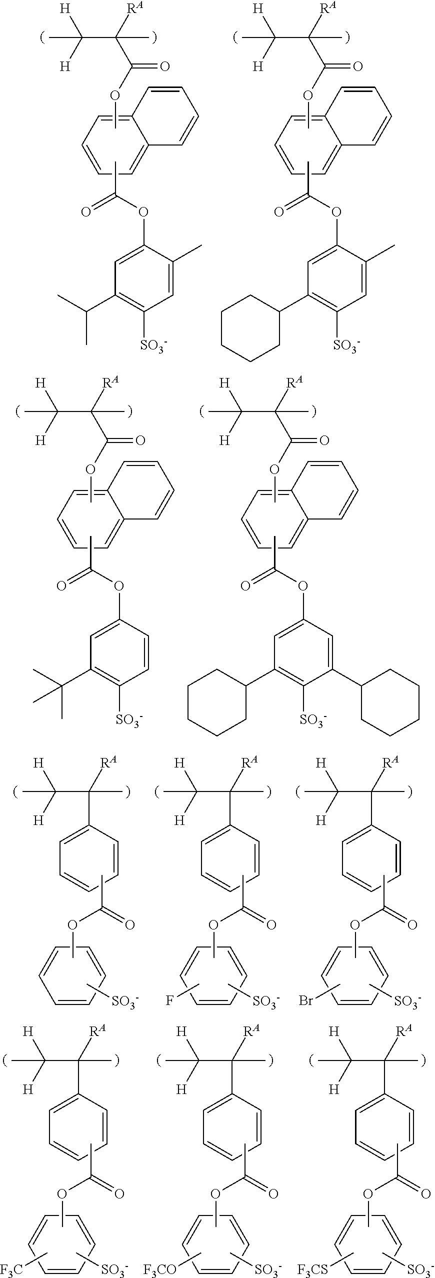

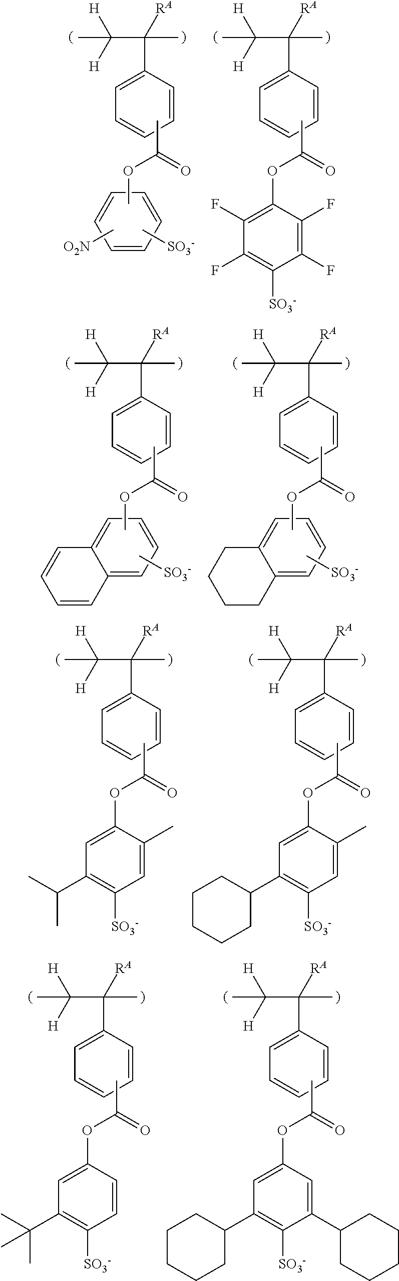

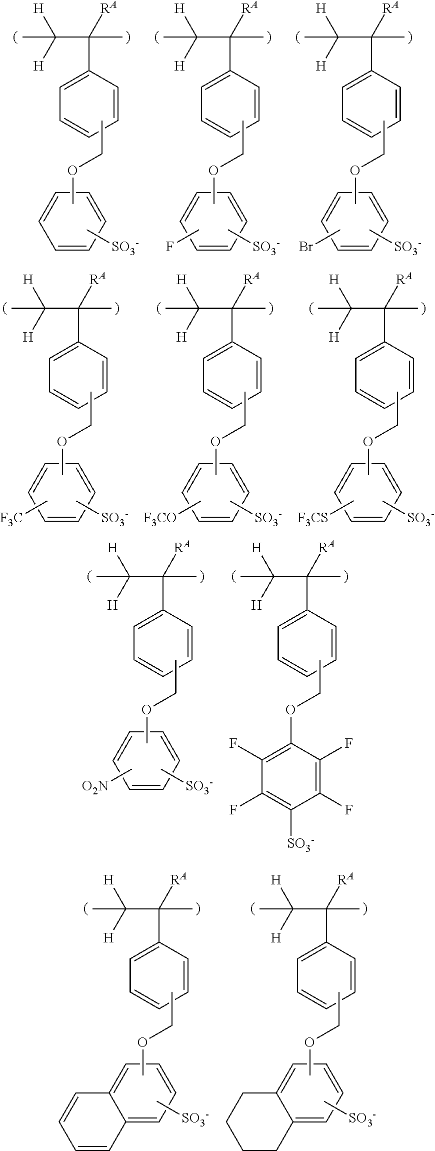

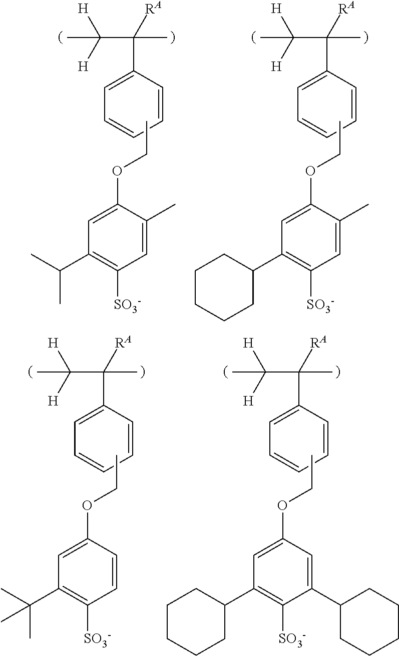

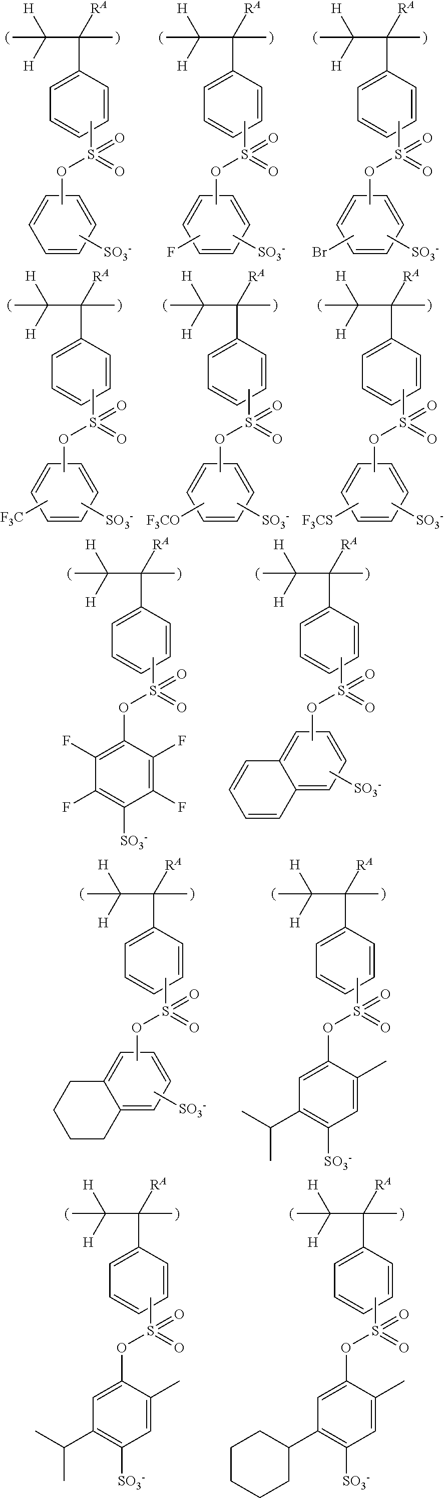

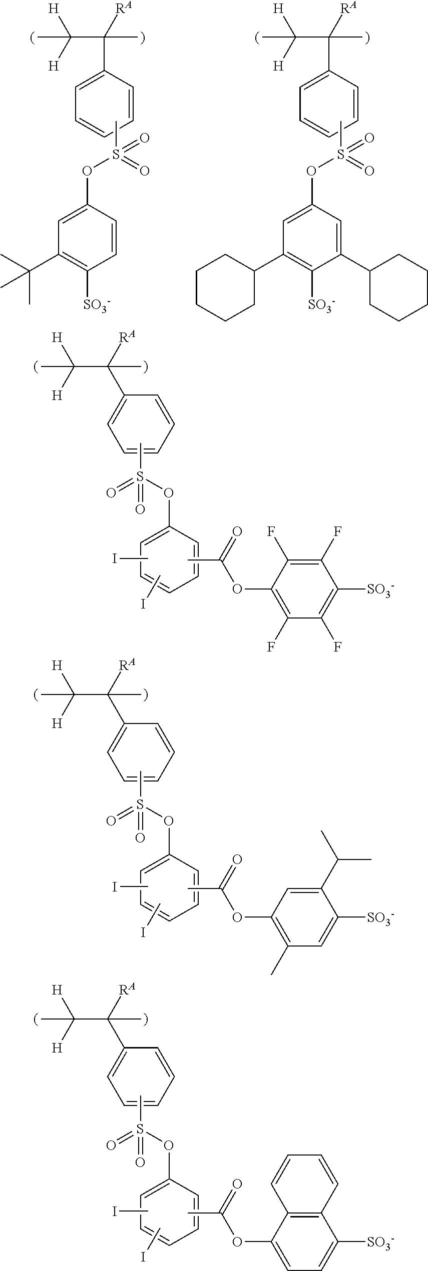

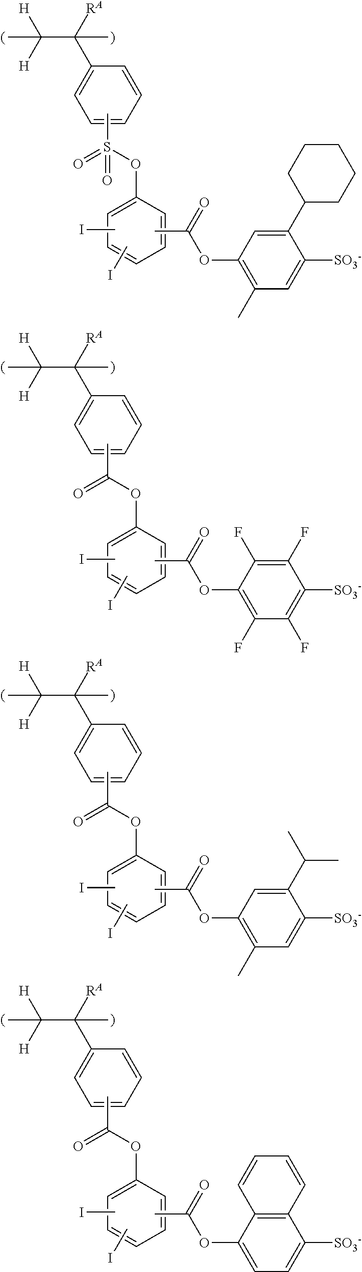

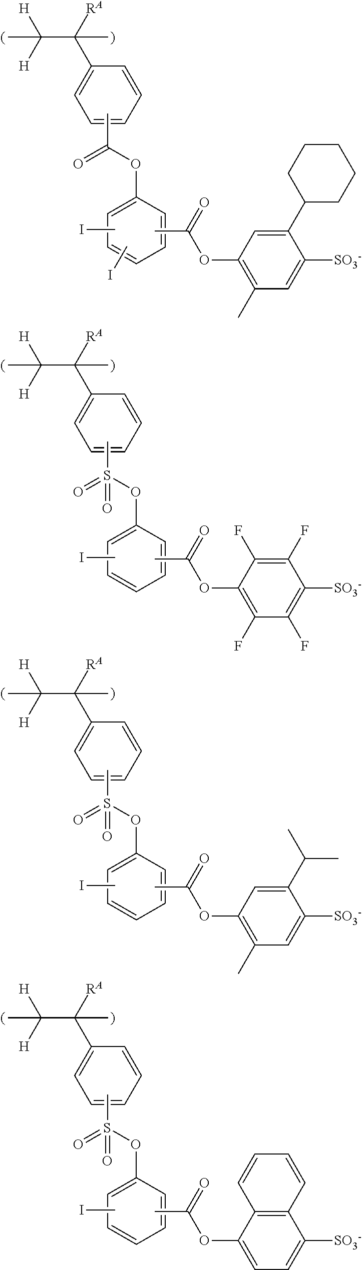

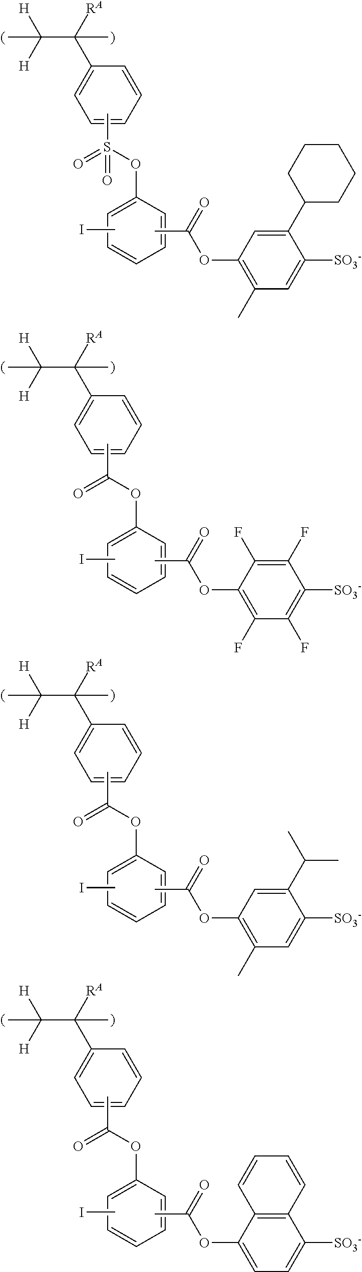

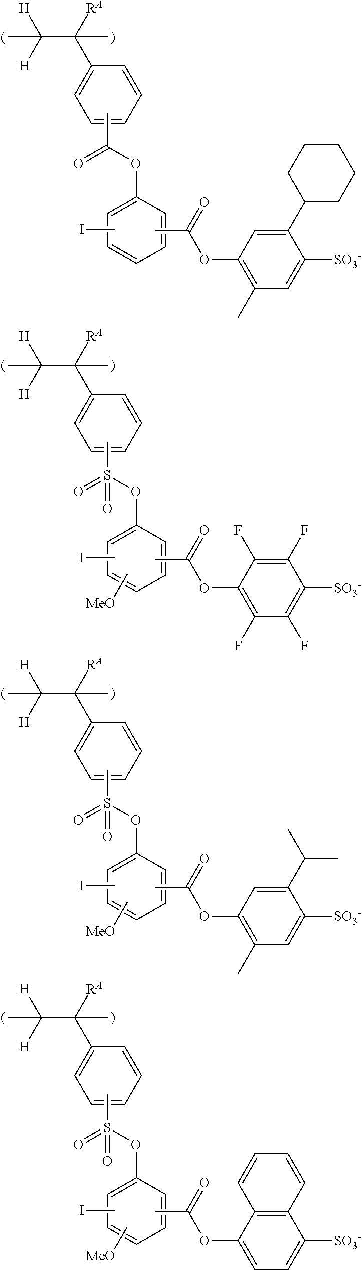

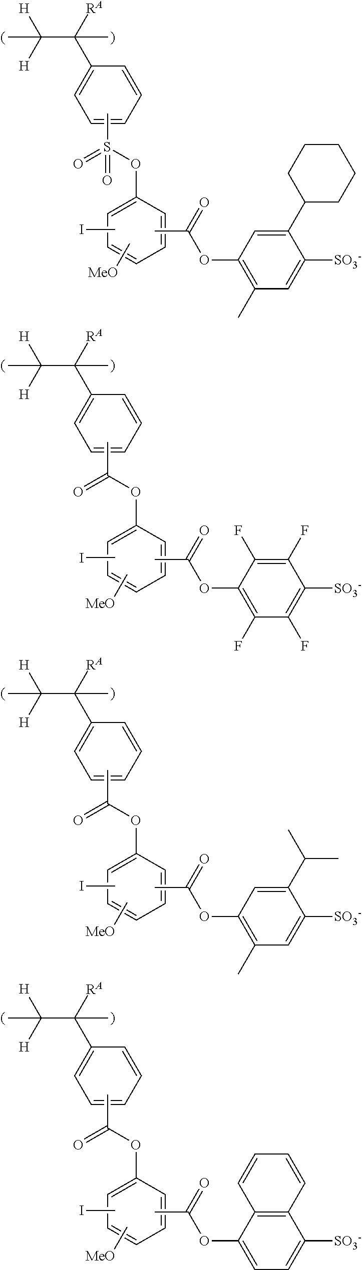

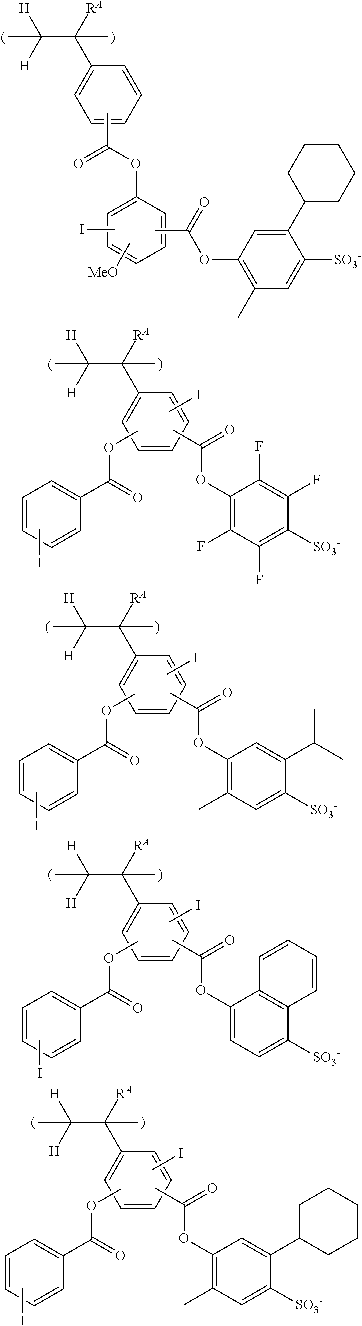

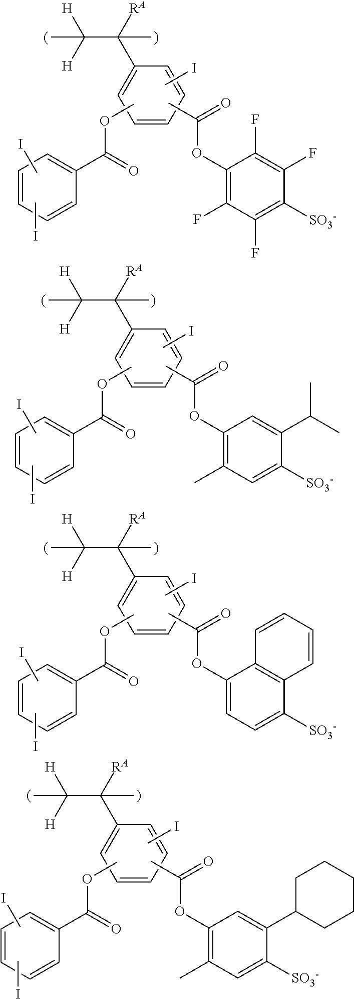

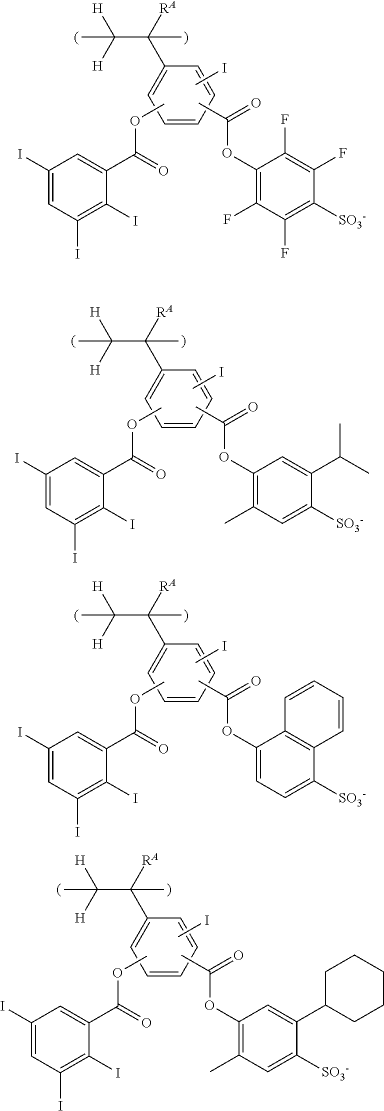

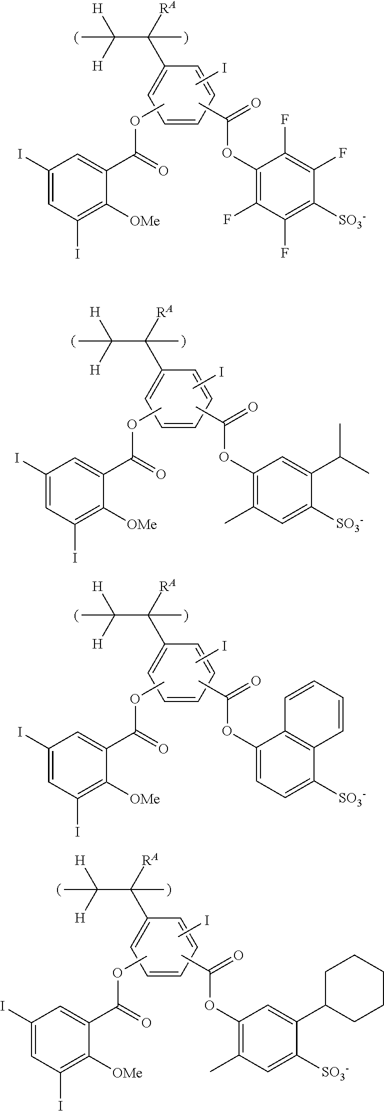

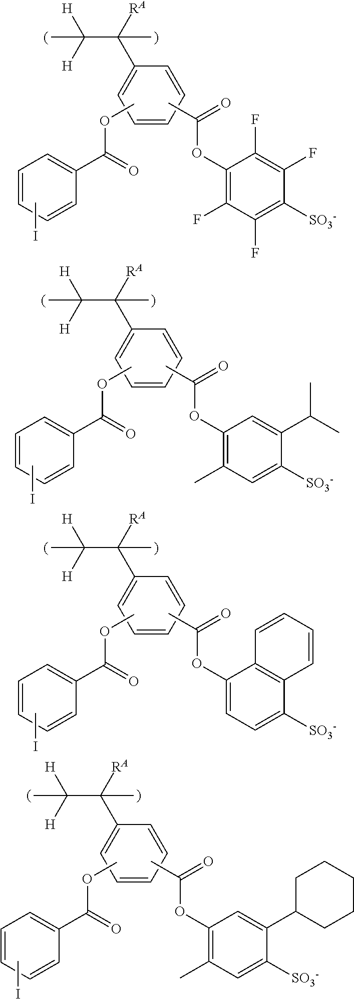

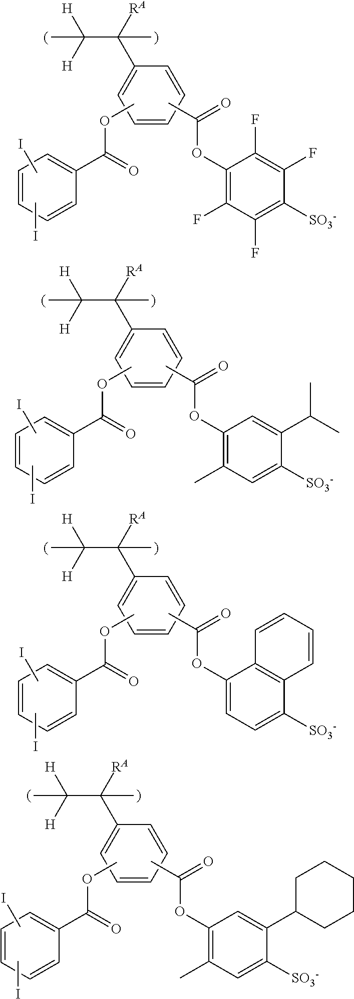

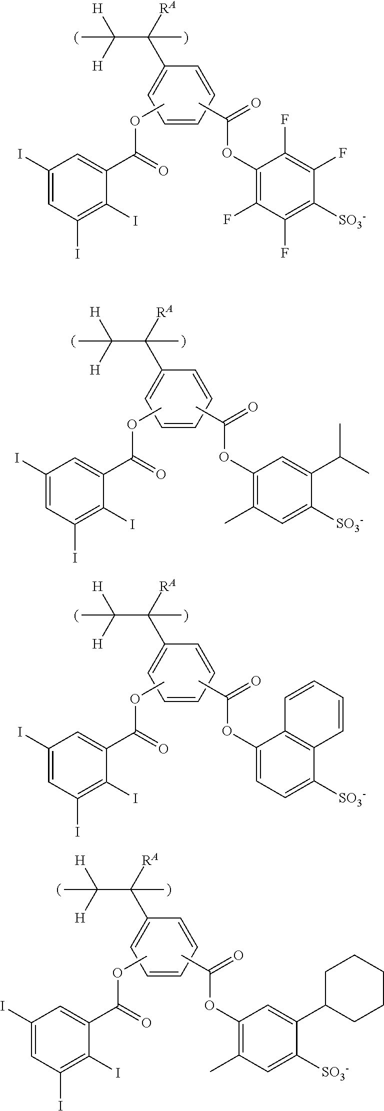

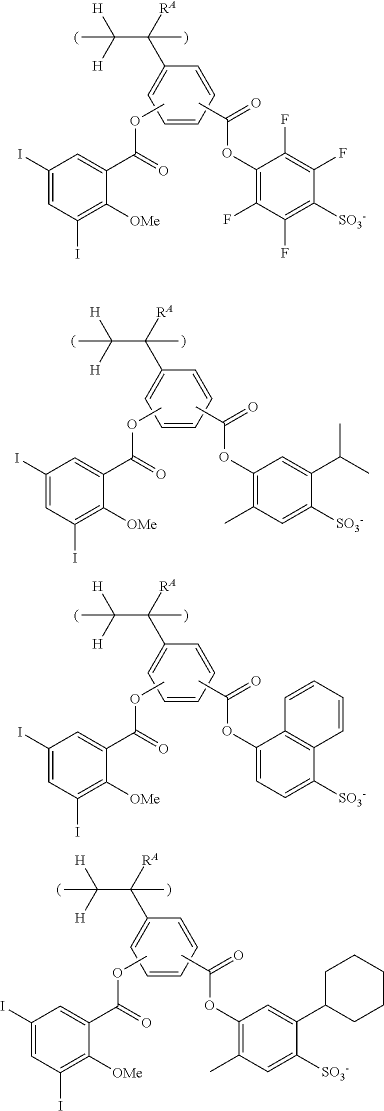

Specific examples of the anion of the repeating unit-c5 include, but are not limited to, the following. Note that in the following formulae, R^{A} is as defined above.

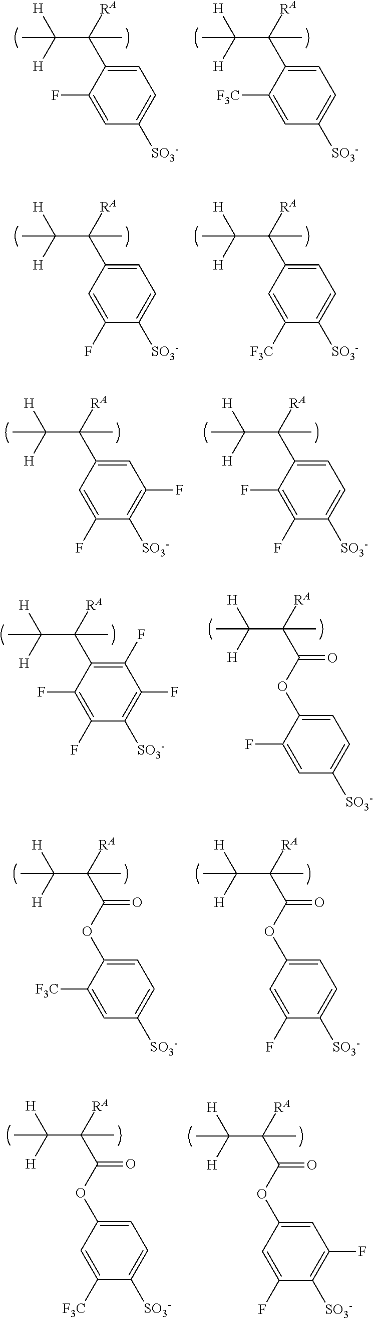

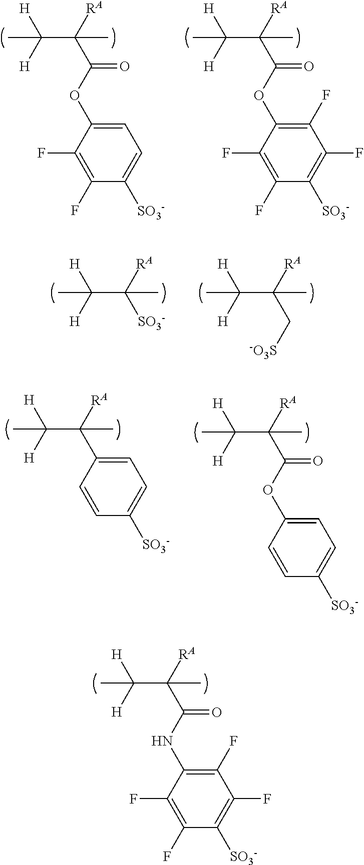

In the general formulae (c2) to (c5), A⁺ represents an onium cation. The onium cation includes a sulfonium cation, an iodonium cation, an ammonium cation, etc., and is preferably a sulfonium cation or an iodonium cation. Specific examples of the sulfonium cation include those exemplified as specific examples of the sulfonium cation in the general formula (1B), those described in paragraphs [0102] to [0125] of JP2024-003744A, those described in paragraphs [0044] to [0049] of WO2024/128017A1, and those described in paragraphs [0035] to [0046] of JP7491173B2, but are not limited thereto.

For the sulfonium cation, the sulfonium cation represented by the following general formula (d3) is also preferable.

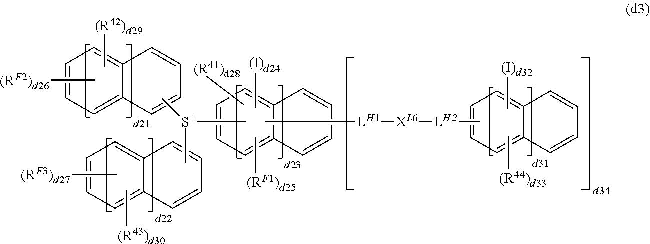

In the general formula (d3), “d21” represents 0 or 1. When “d21” is 0, the structure is a benzene ring, and when “d21” is 1, it is a naphthalene ring; however, from the viewpoint of solvent solubility, it is preferable that “d21” is 0, giving a benzene ring. “d22” represents 0 or 1. When “d22” is 0, the structure is a benzene ring, and when “d22” is 1, it is a naphthalene ring; however, from the viewpoint of solvent solubility, it is preferable that “d22” is 0, giving a benzene ring. “d23” represents 0 or 1. When “d23” is 0, the structure is a benzene ring, and when “d23” is 1, it is a naphthalene ring; however, from the viewpoint of solvent solubility, it is preferable that “d23” is 0, giving a benzene ring.

In the general formula (d3), “d24” represents an integer from 0 to 4. The greater the number of iodine atoms in the cationic structure, the greater the absorption, particularly of EUV, but the poorer the solvent solubility, and there is a risk of precipitation in the resist composition, so “d24” is preferably 0, 1, 2 or 3, and more preferably 0, 1 or 2.

In the general formula (d3), “d25” represents an integer from 0 to 4. From the viewpoint of raw material procurement, “d25” is preferably 0, 1, 2 or 3, and more preferably 0, 1 or 2. “d26” represents an integer from 0 to 6. From the viewpoint of raw material procurement, “d26” is preferably 0, 1, 2 or 3, and more preferably 0, 1 or 2. “d27” represents an integer from 0 to 6. From the viewpoint of raw material procurement, “d27” is preferably 0, 1, 2 or 3, and more preferably 0, 1 or 2.

In the general formula (d3), “d28” represents 0, 1 or 2. From the viewpoint of raw material procurement, “d28” is preferably 0 or 1. “d29” represents 0, 1 or 2. From the viewpoint of raw material procurement, “d29” is preferably 0 or 1. “d30” represents 0, 1 or 2. From the viewpoint of raw material procurement, “d30” is preferably 0 or 1.

In the general formula (d3), “d31” represents 0 or 1. When “d31” is 0, the structure is a benzene ring, and when “d31” is 1, it is a naphthalene ring; however, from the viewpoint of solvent solubility, it is preferable that “d31” is 0, giving a benzene ring.

In the general formula (d3), “d32” represents an integer from 0 to 4. The greater the number of iodine atoms in the cationic structure, the greater the absorption, particularly of EUV, but the poorer the solvent solubility, and there is a risk of precipitation in the resist composition, so “d32” is preferably 0, 1, 2 or 3, and more preferably 0, 1 or 2.

In the general formula (d3), “d33” represents 0, 1 or 2. From the viewpoint of raw material procurement, “d33” is preferably 0 or 1. “d34” represents 0, 1 or 2. From a synthetic standpoint, “d34” is preferably 0 or 1.

However, when “d21” is 0, 0≤d26+d29≤4, and when “d21” is 1, 0≤d26+d29≤6. When “d22” is 0, 0≤d27+d30≤4, and when “d22” is 1, 0≤d27+d30≤6. When “d23” is 0, 1≤d24+d25+d28+d34≤4, and when “d23” is 1, 1≤d24+d25+d28+d34≤6. When “d31” is 0, 0≤d32+d33≤4, and when “d31” is 1, 0≤d32+d33≤6. Also, 1≤d24+d32.

In the general formula (d3), R^{F1} to RF³ each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Of these, a trifluoromethyl group, a trifluoromethoxy group, or a trifluorothiomethoxy group is preferable. When “d25” is 2 or more, each R^{F1} may be the same as or different from each other, when “d26” is 2 or more, each RF² may be the same as or different from each other, and when “d27” is 2 or more, each RF³ may be the same as or different from each other.

In the general formula (d3), R⁴¹ to R⁴⁴ represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl groups and the hydrocarbyl moieties of the hydrocarbyloxy groups and the hydrocarbylthio groups may be saturated or unsaturated and may be linear, branched, or cyclic. Specific examples thereof include the same ones as those exemplified as the hydrocarbyl group represented by R¹ in the explanation of the general formula (1A). In addition, some or all of the hydrogen atoms of the hydrocarbyl group and the hydrocarbyl moiety of the hydrocarbyloxy group and the hydrocarbylthio group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Also, when “d28” is 2, two R⁴¹ may be the same as or different from each other, and two R⁴¹ may be bonded to each other to form a ring together with the carbon atoms to which they are bonded, when “d29” is 2, two R⁴² may be the same as or different from each other, and two R⁴² may be bonded to each other to form a ring together with the carbon atoms to which they are bonded, when “d30” is 2, two R⁴³ may be the same as or different from each other, and two R⁴³ may be bonded to each other to form a ring together with the carbon atoms to which they are bonded, and when “d33” is 2, two R⁴⁴ may be the same as or different from each other, and two R⁴⁴ may be bonded to each other to form a ring together with the carbon atoms to which they are bonded. Specific examples of the ring formed in this case include a cyclopropane ring, a cyclobutane ring, a cyclopentane ring, a cyclohexane ring, a norbornane ring, an adamantane ring, etc. In addition, some or all of the hydrogen atoms of the above-described rings may be substituted with a group containing a heteroatom such as an oxygen atom, a sulfur atom, a nitrogen atom, or a halogen atom, and some of the —CH₂— of the above-described rings may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Moreover, the aromatic rings directly bonded to S⁺ in the sulfonium cation represented by the general formula (d3) may form a ring together with the S⁺ by bonding to each other. In this case, specific examples of the ring structure include those represented by the following formulae, etc.

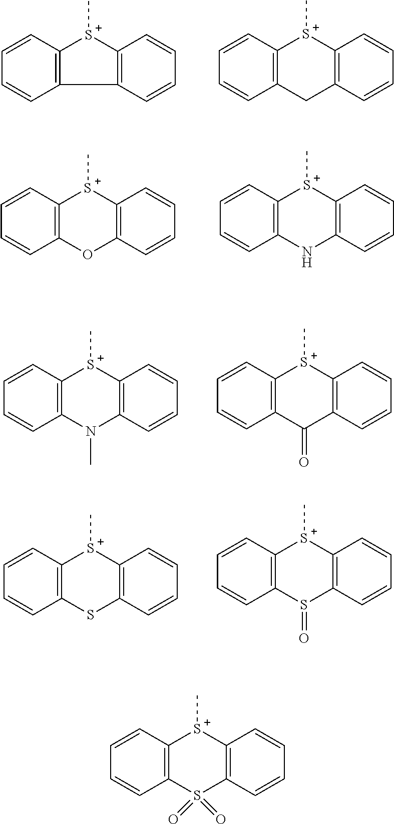

In the formulae, a broken line represents an PGP attachment point.

In the general formula (d3), L^{H1} and L^{H2} each independently represent a single bond, an ether bond, an ester bond, an amide bond, a sulfonate ester bond, a sulfonate amide bond, a carbonate bond, or a carbamate bond. Of these, L^{H1} is preferably a single bond, an ether bond, an ester bond, or a sulfonate ester bond, and more preferably an ester bond or a sulfonate ester bond. L^{H2} is preferably a single bond, an ether bond, or an ester bond, and more preferably a single bond.

In the general formula (d3), X^{L6} represents a single bond, or a hydrocarbylene group having 1 to 40 carbon atoms and optionally containing a heteroatom. The hydrocarbylene group may be either linear, branched, or cyclic, and specific examples thereof include an alkanediyl group, a cyclic saturated hydrocarbylene group, an arylene group, etc. Specific examples of the heteroatom include an oxygen atom, a nitrogen atom, a sulfur atom, etc. Specific examples of the hydrocarbylene group having 1 to 40 carbon atoms and optionally containing a heteroatom represented by X^{L6} are X^{L}-0 to X^{L}-58 exemplified as specific examples of the hydrocarbylene group having 1 to 40 carbon atoms and optionally containing a heteroatom represented by X^{L1} in the explanation of the general formula (1A). Of these, X^{L}-0 to X^{L}-22, X^{L}-29 to X^{L}-34, and X^{L}-47 to X^{L}-58 are preferable.

The sulfonium cation represented by the general formula (d3) is preferably one represented by the following general formula (d3-1).

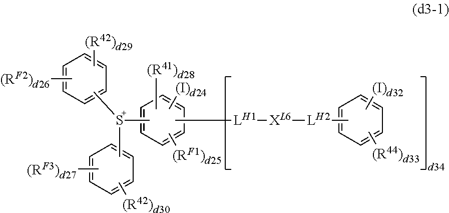

In the formula, “d24” to “d30”, “d32” to “d34”, R^{F1} to R^{F3}, R⁴¹ to R⁴⁴, L^{H1}, L^{H2}, and X^{L} are as defined above.

The sulfonium cation represented by the general formula (d3-1) is preferably one represented by the following general formula (d3-2).

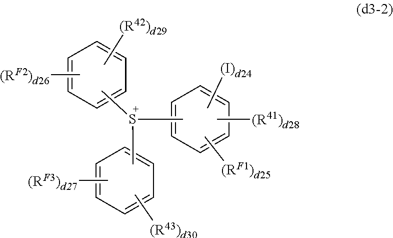

In the formula, “d24” to “d30”, R^{F1} to R^{F3}, and R⁴¹ to R⁴³ are as defied above.

Specific examples of the sulfonium cation represented by the general formula (d3) include, but are not limited to, the following. Note that in the following formulae, Me represents a methyl group.

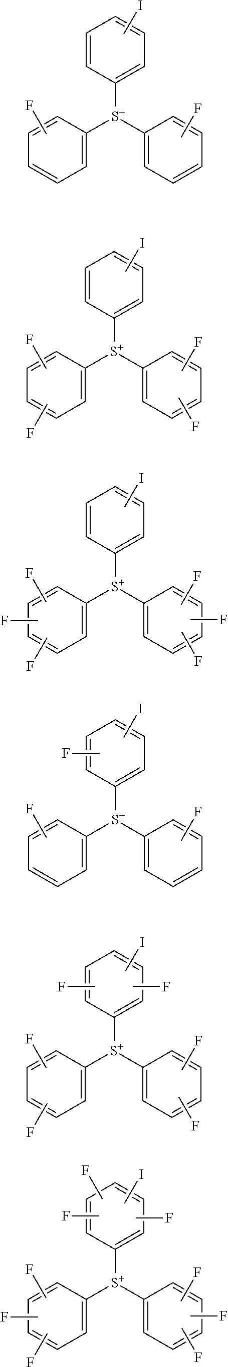

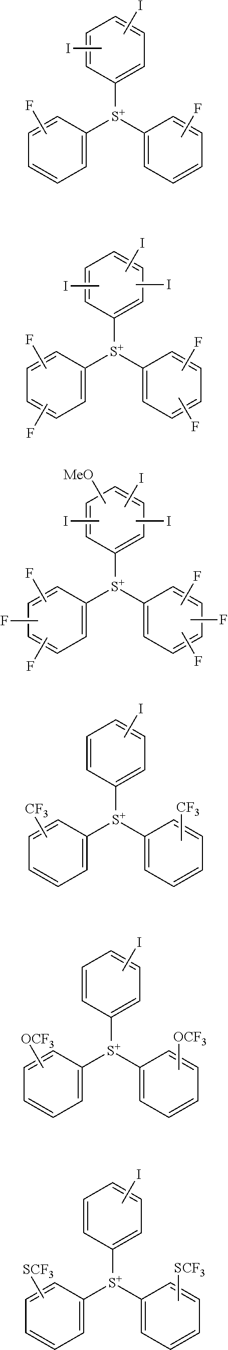

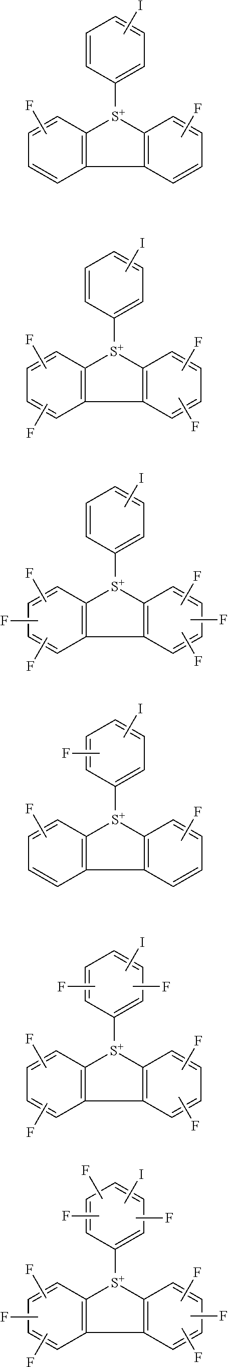

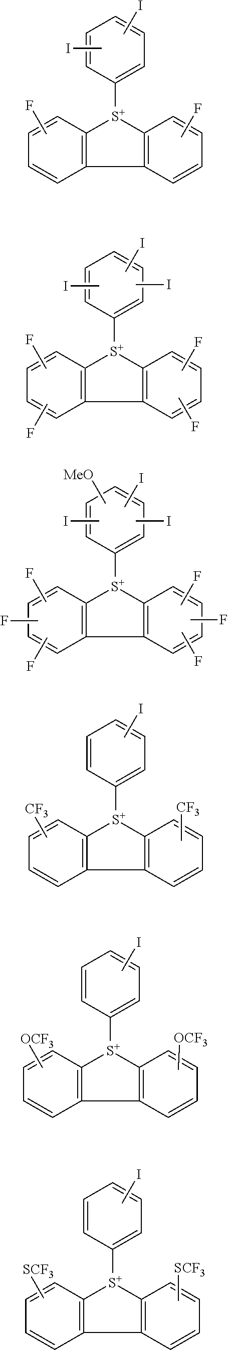

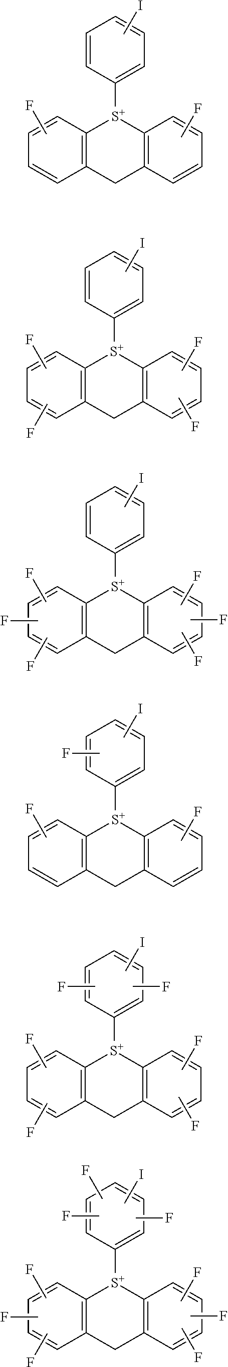

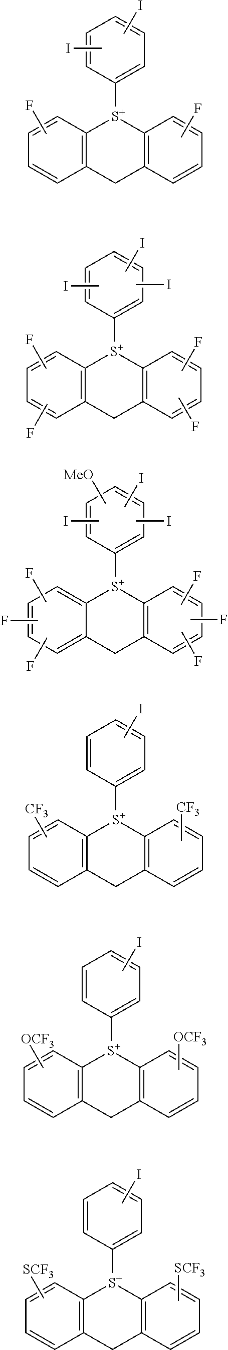

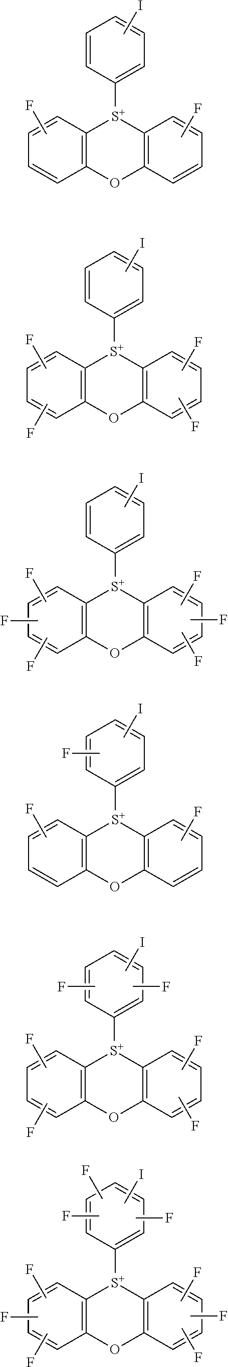

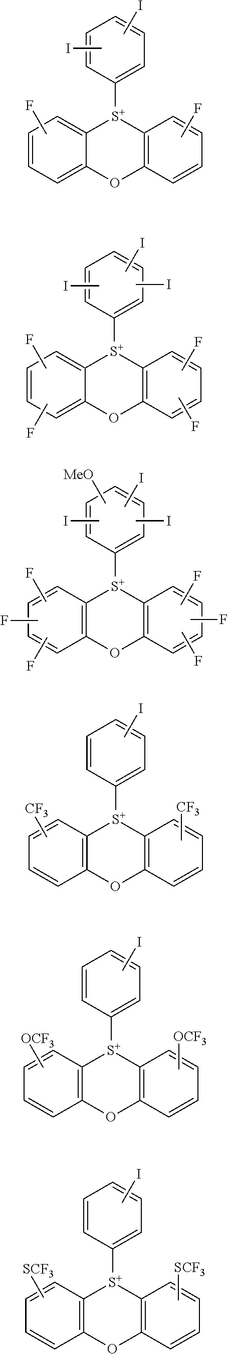

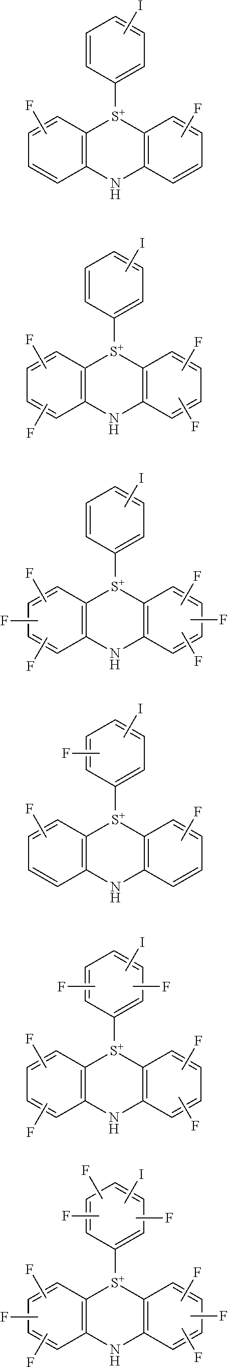

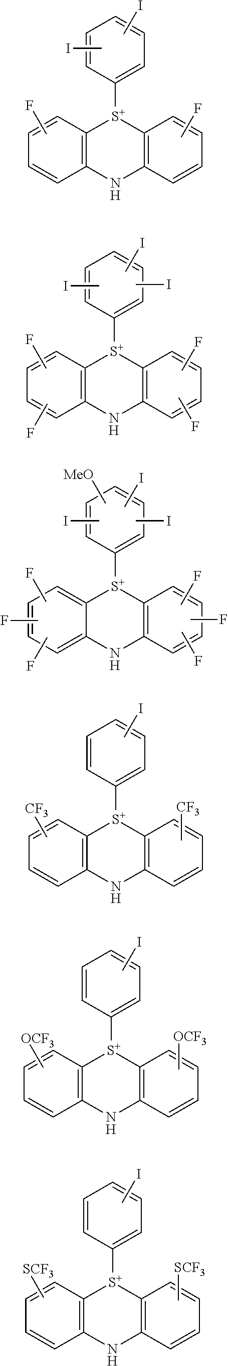

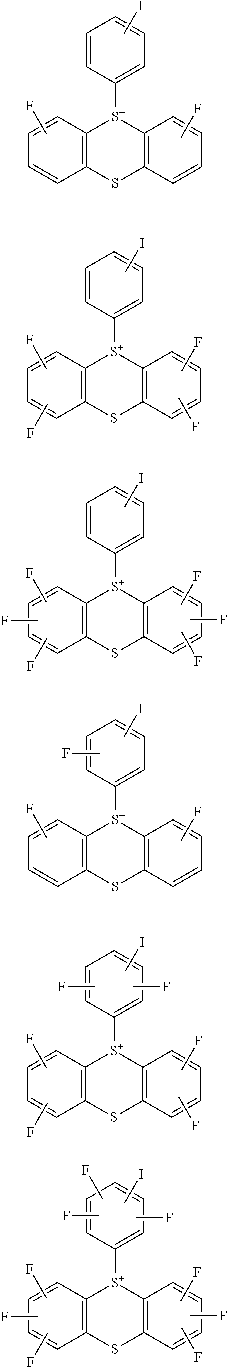

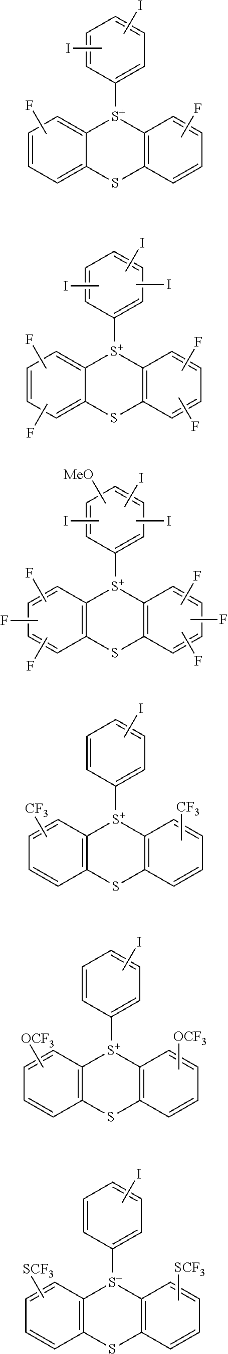

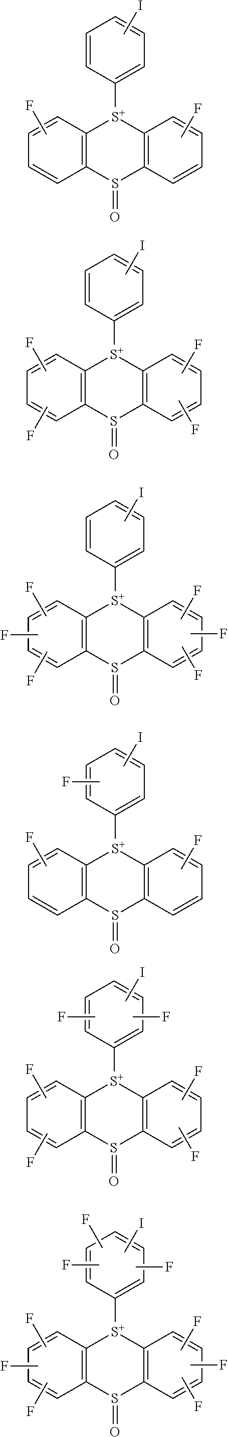

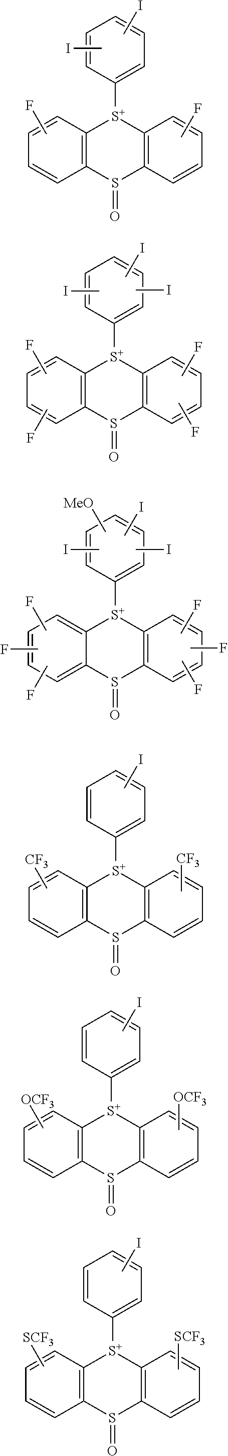

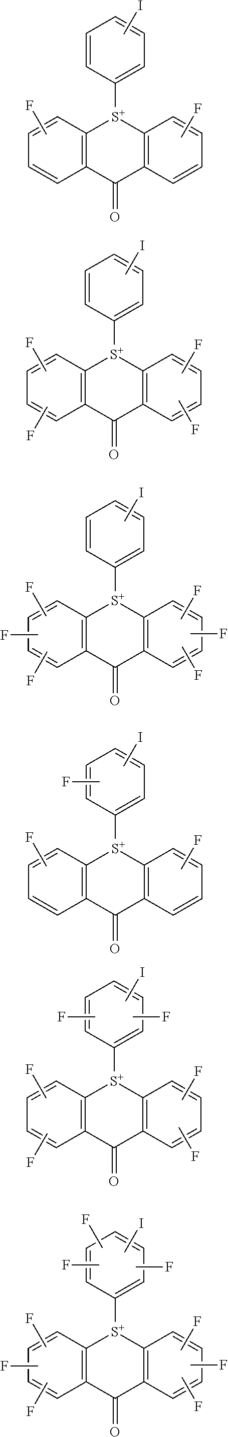

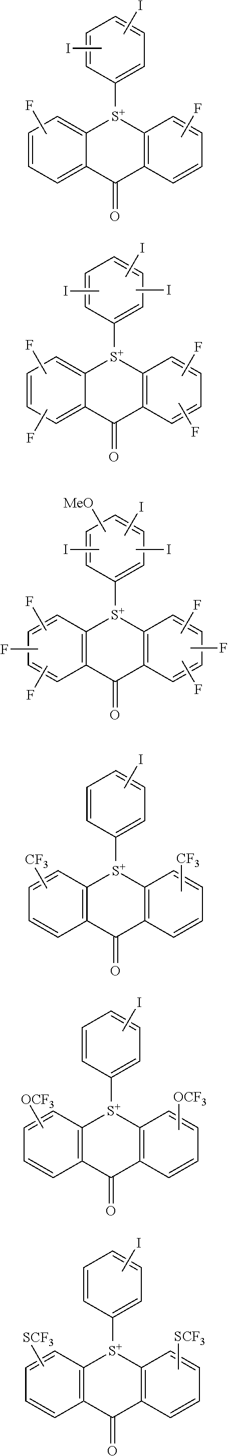

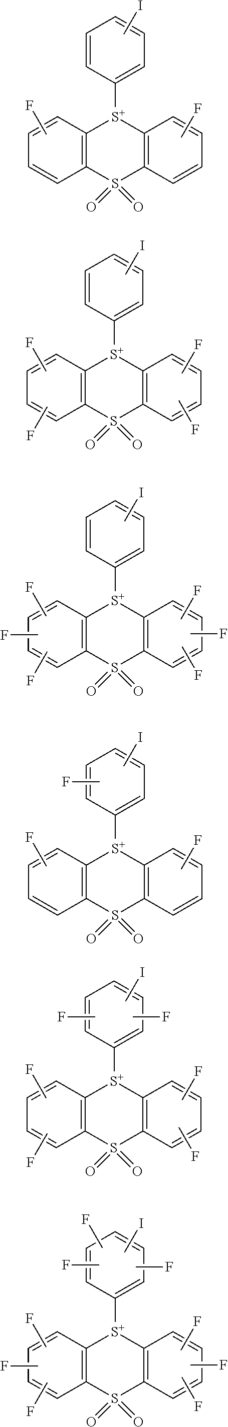

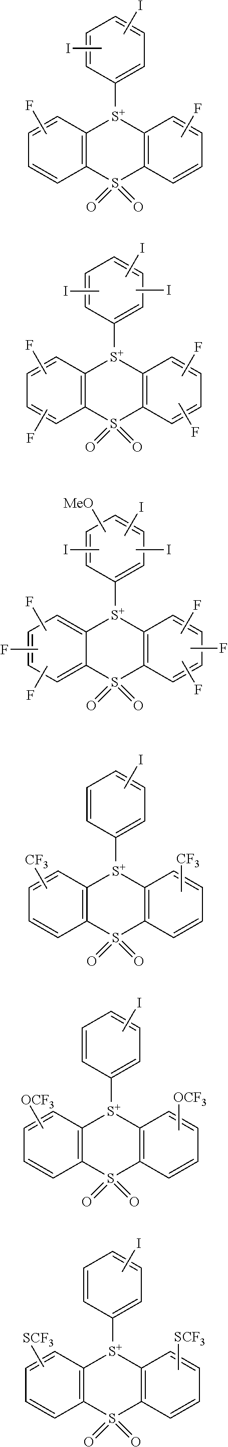

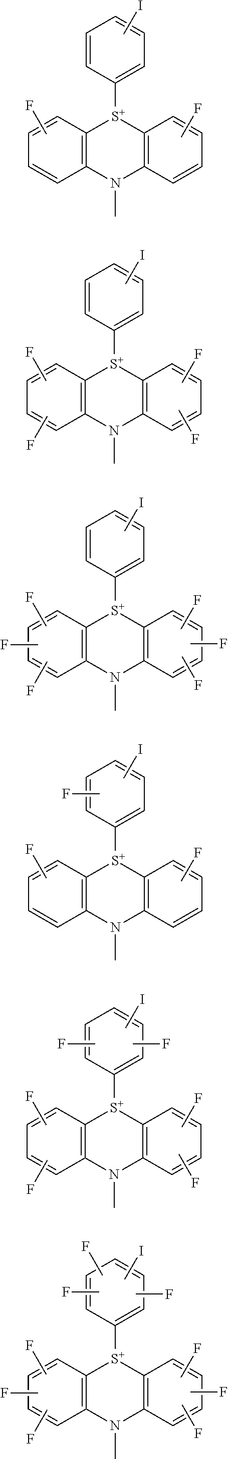

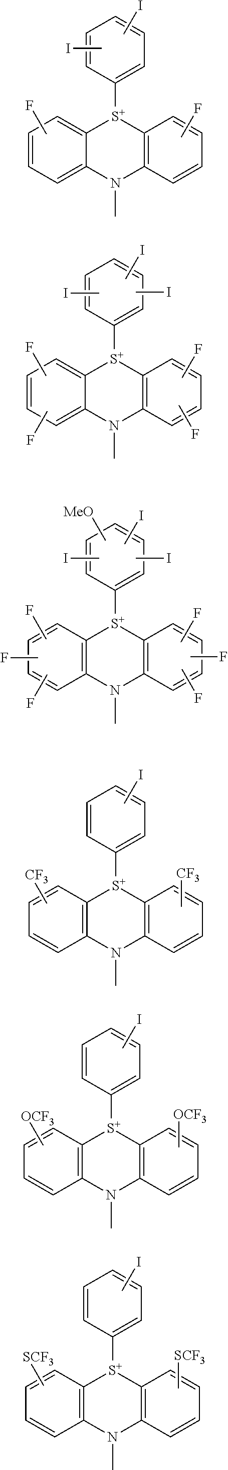

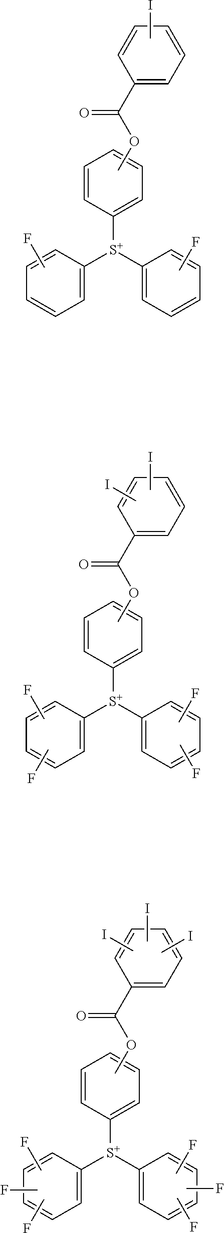

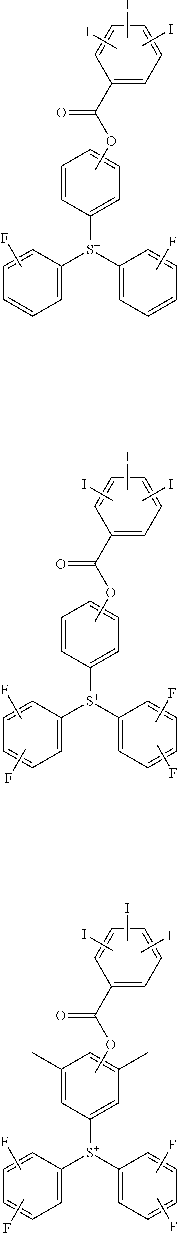

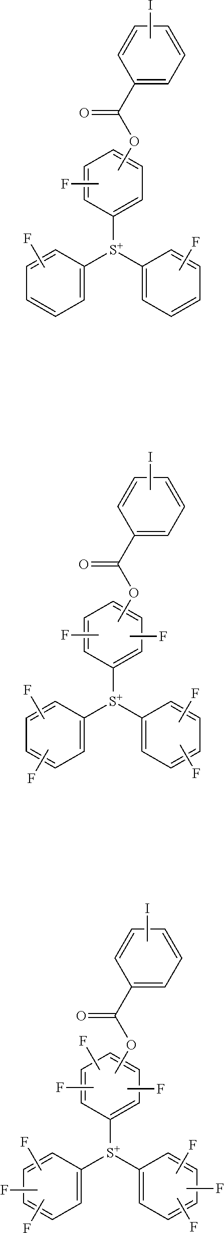

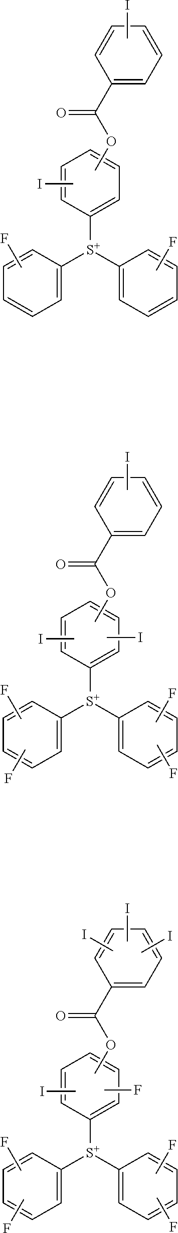

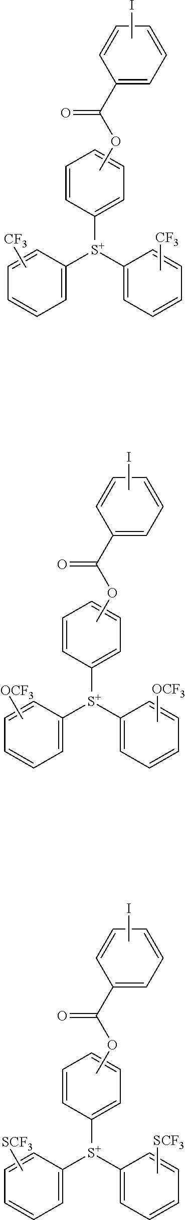

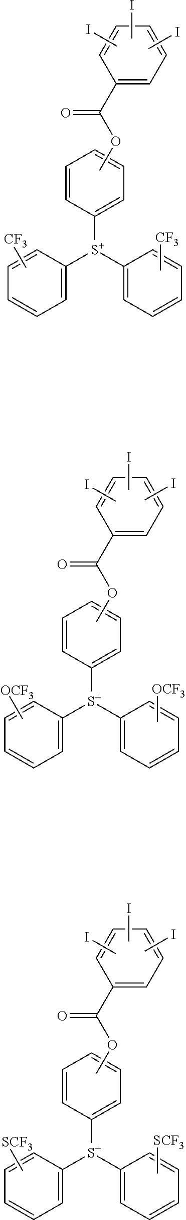

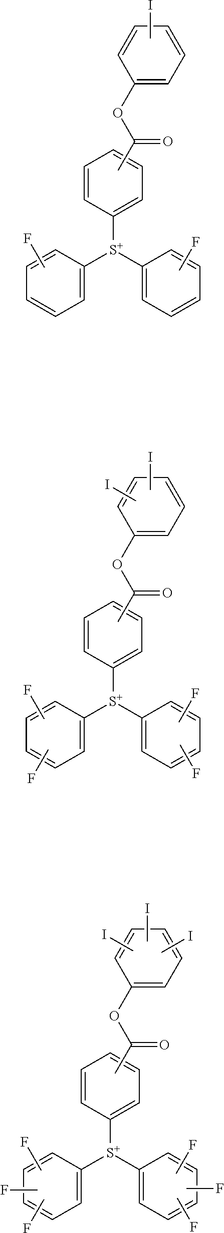

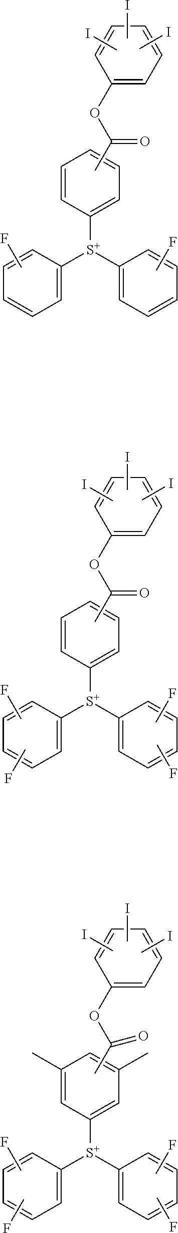

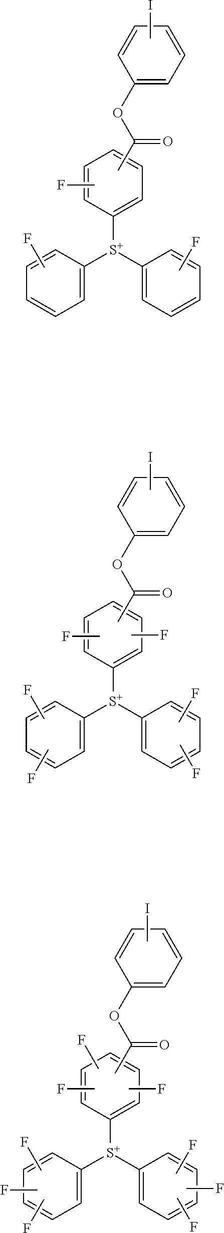

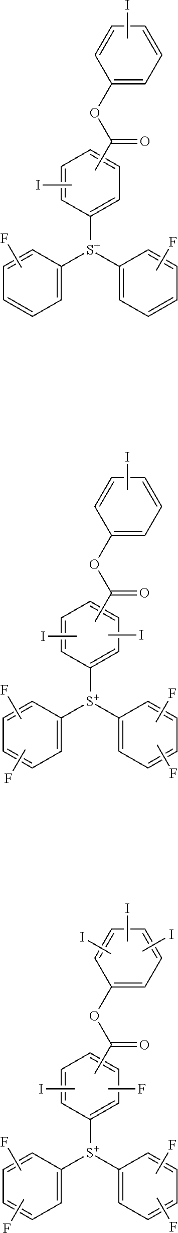

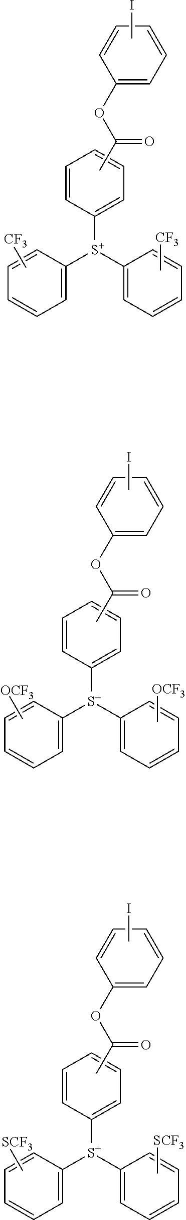

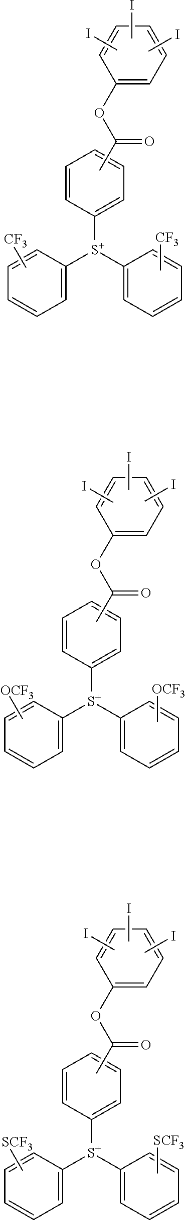

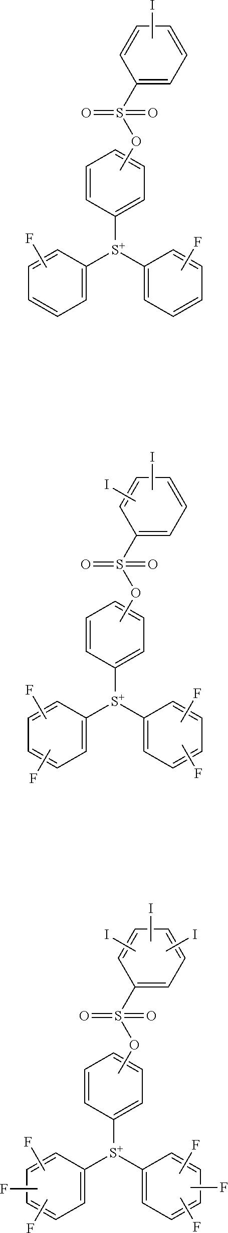

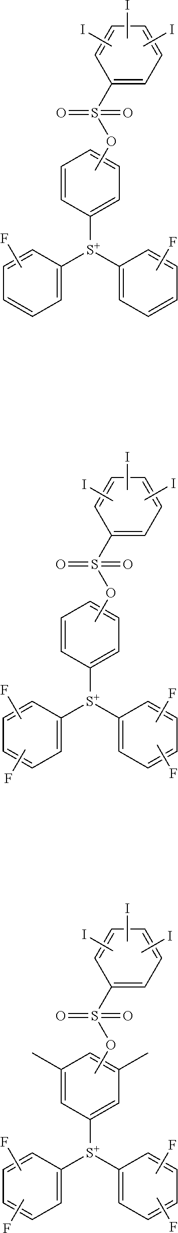

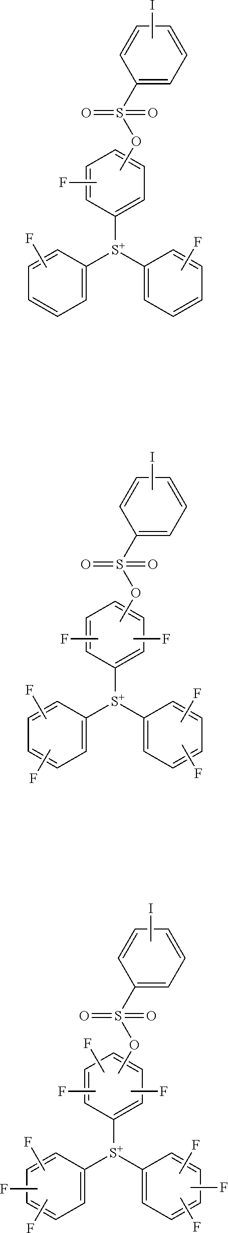

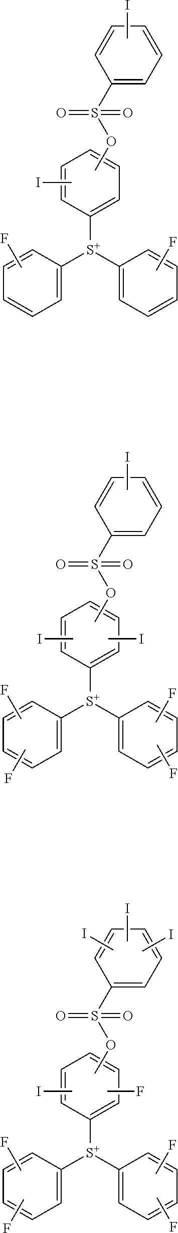

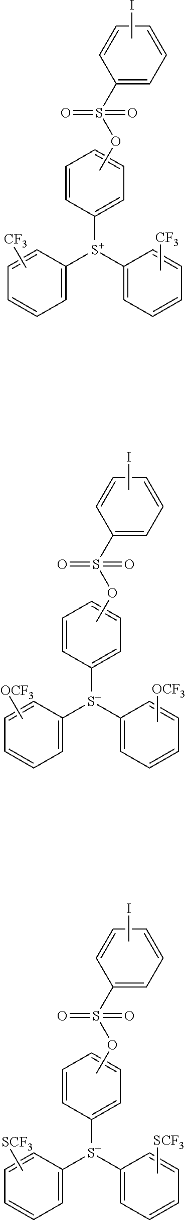

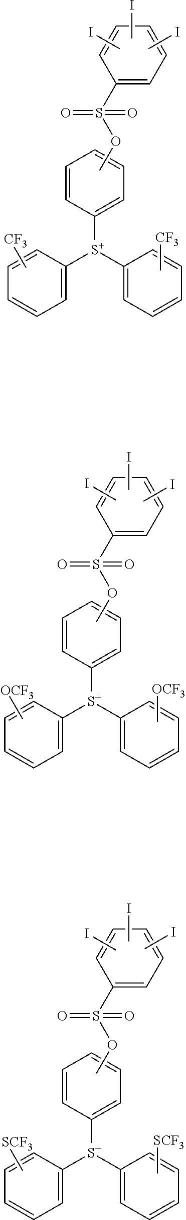

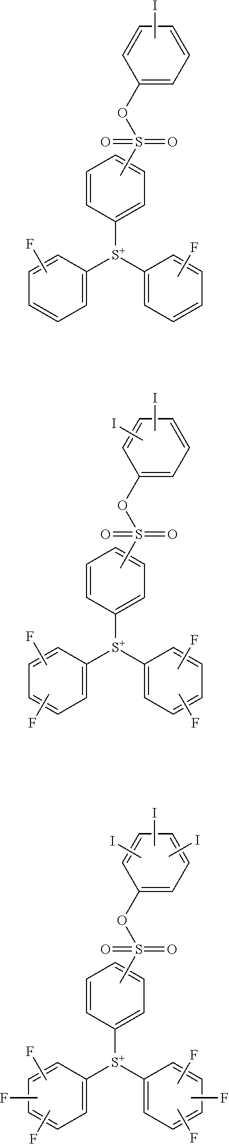

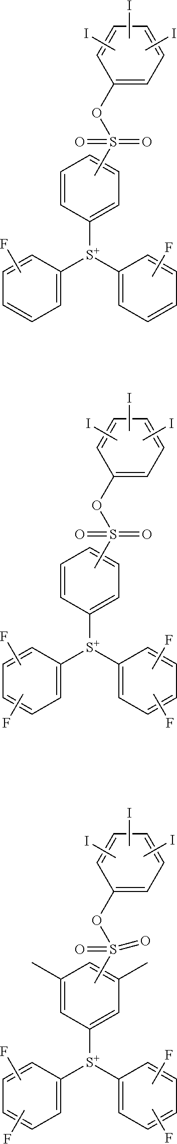

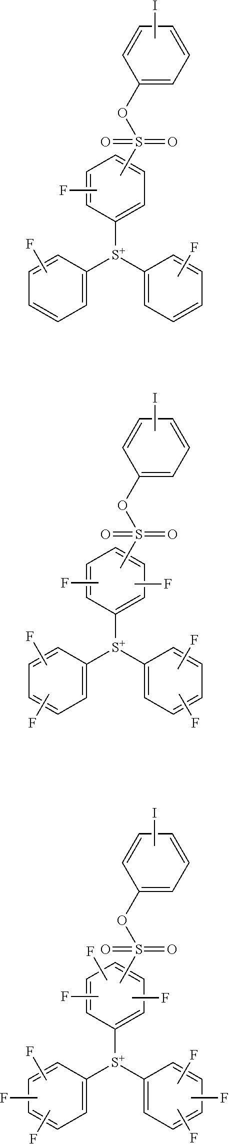

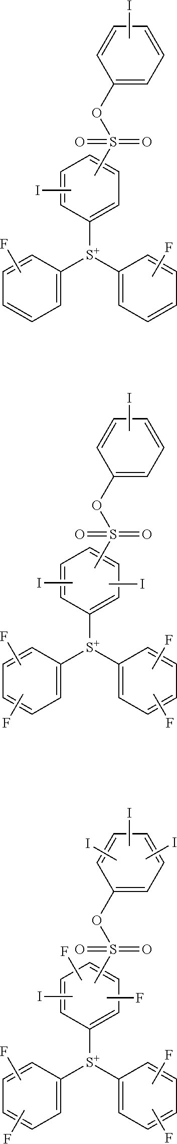

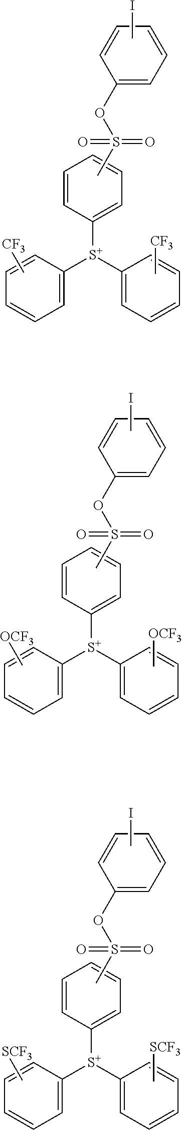

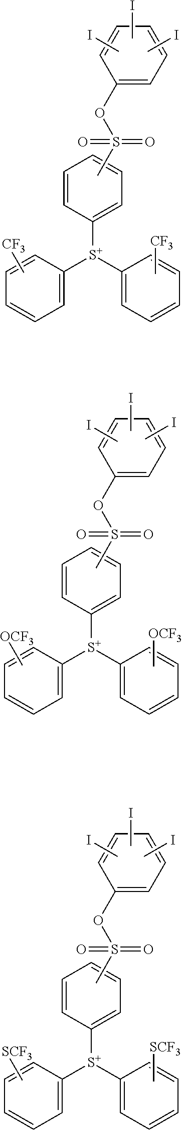

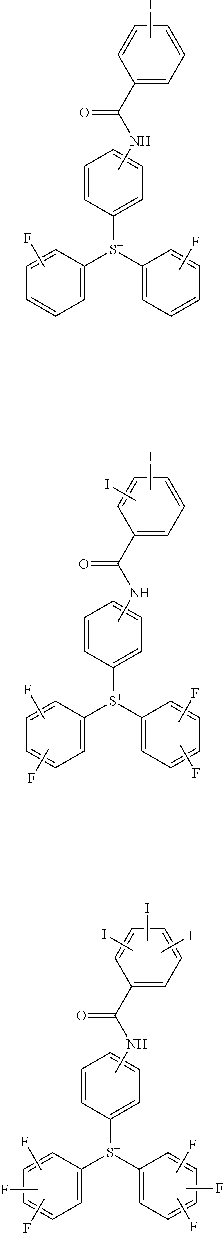

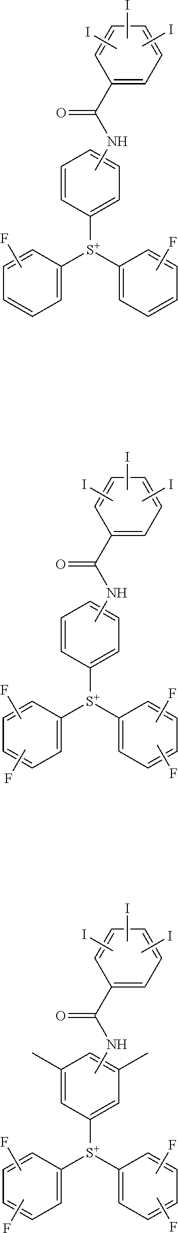

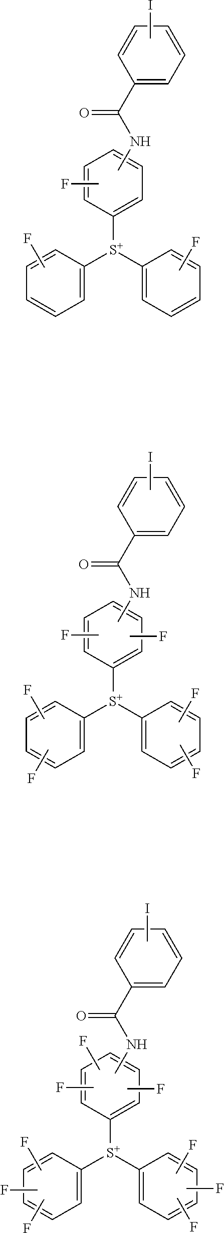

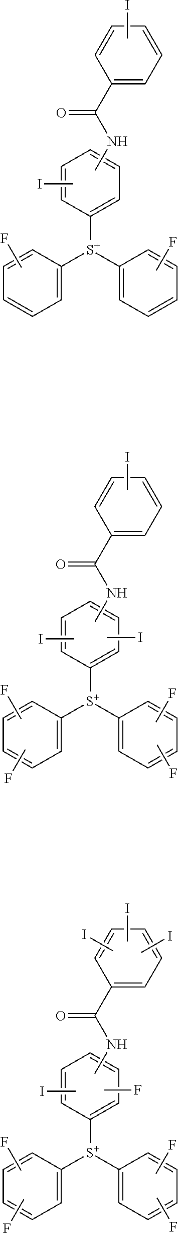

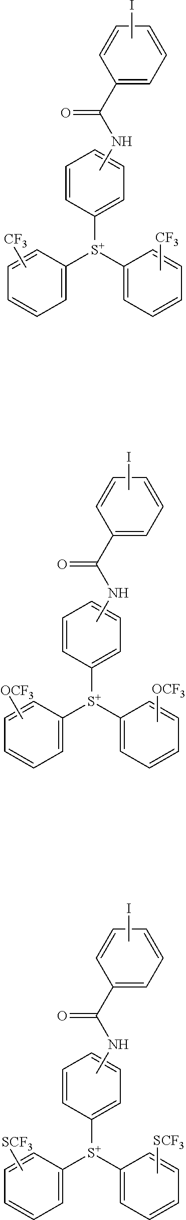

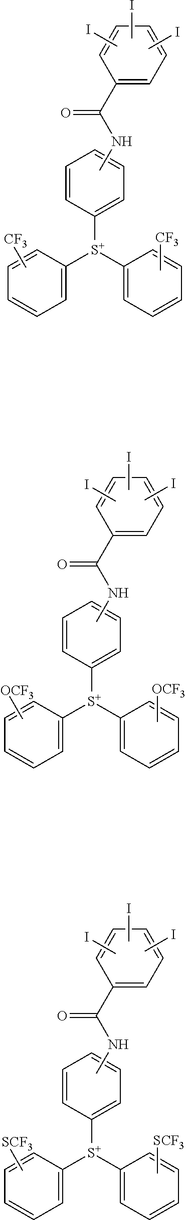

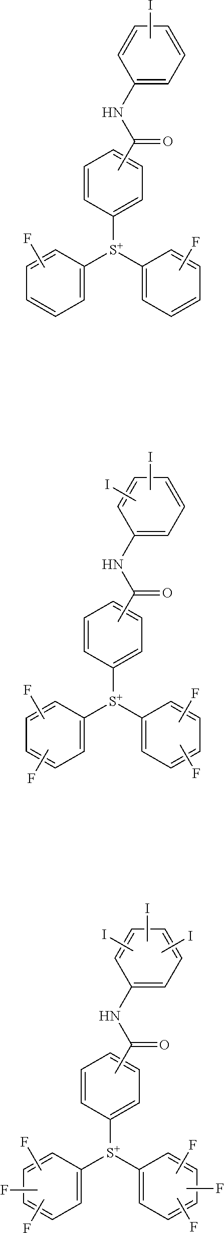

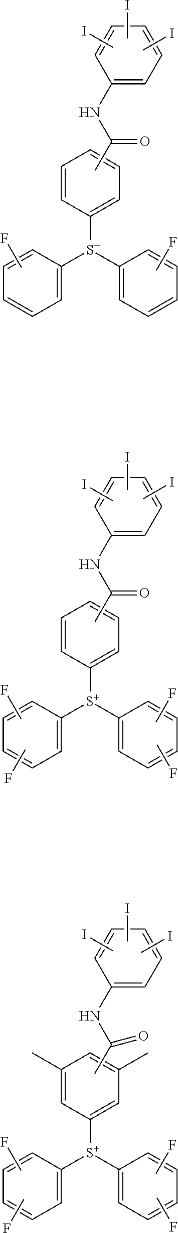

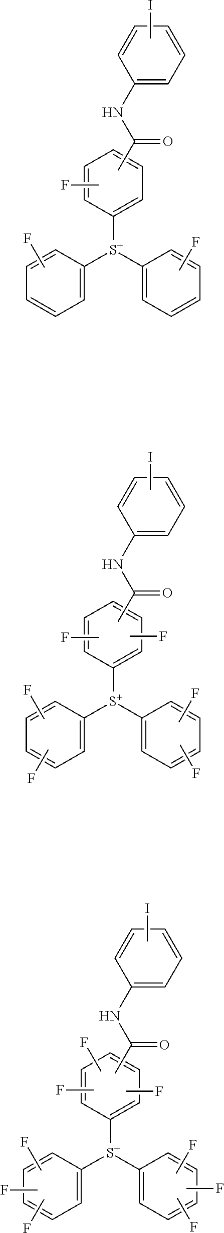

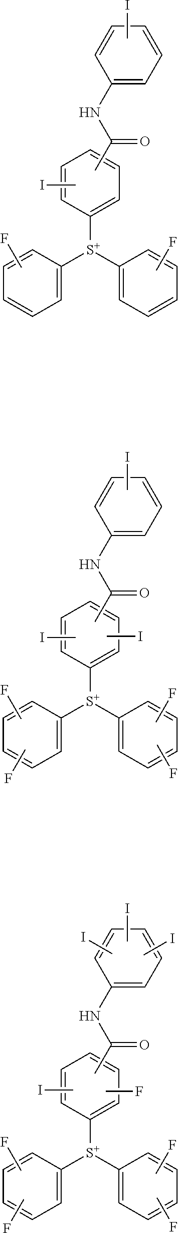

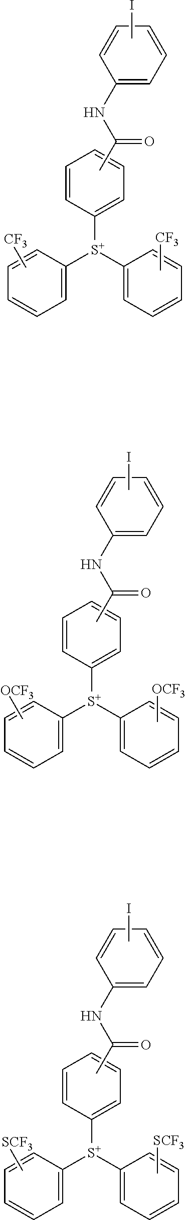

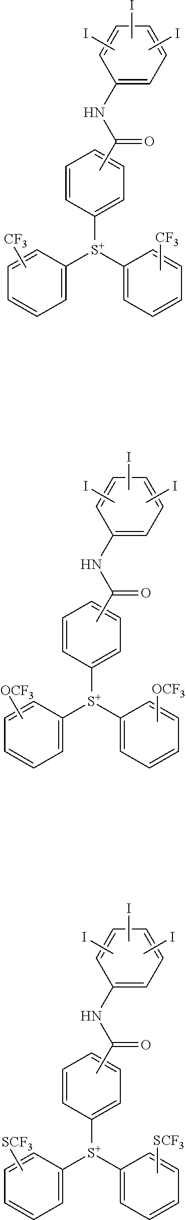

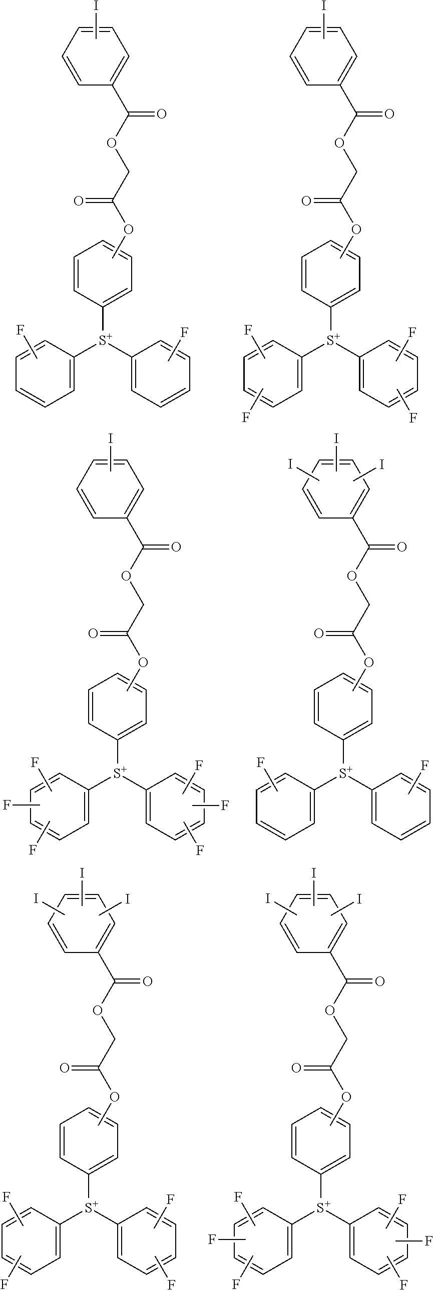

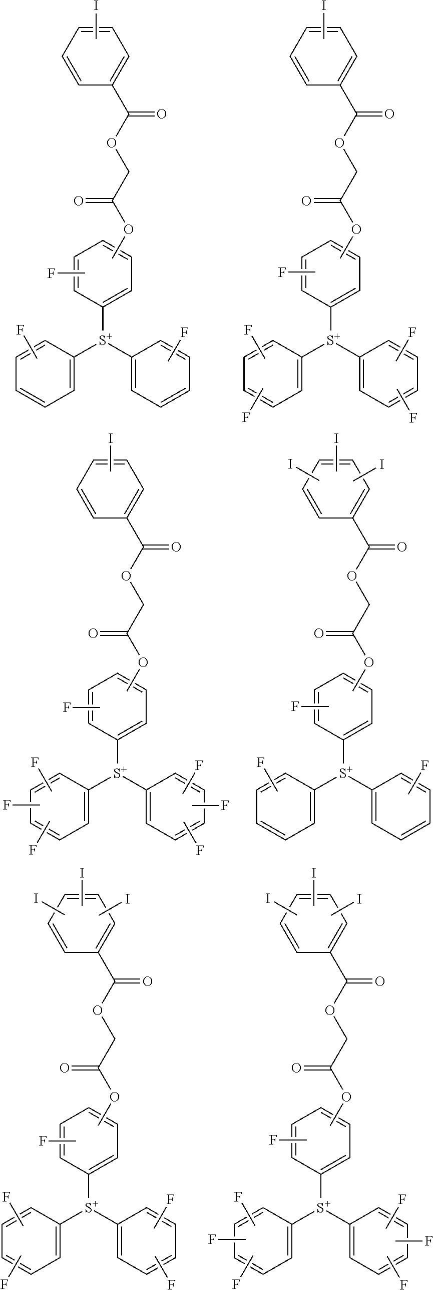

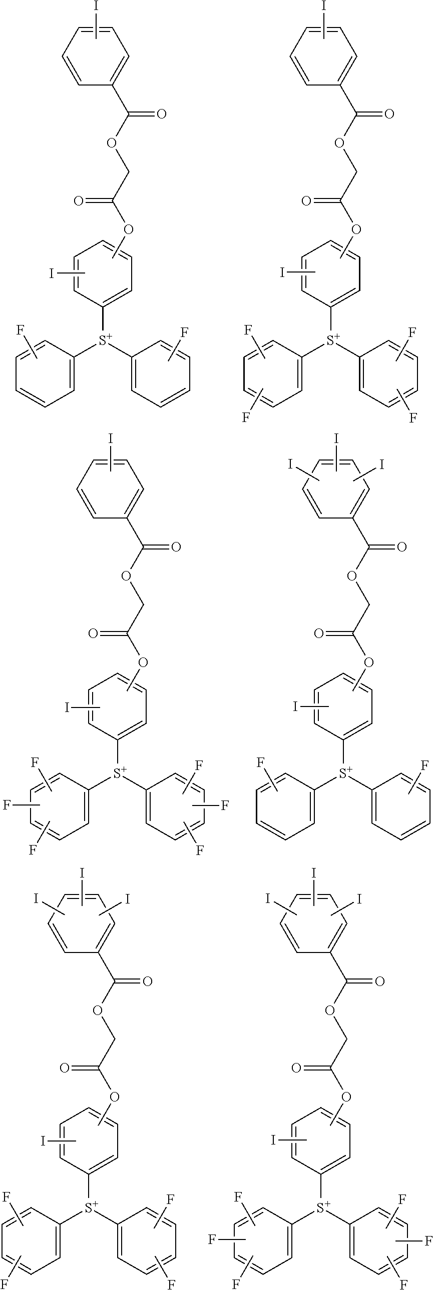

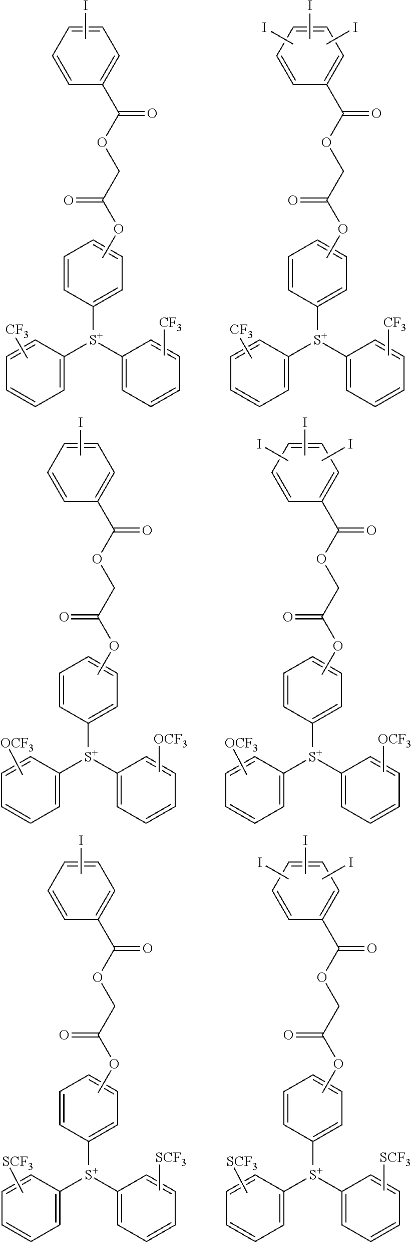

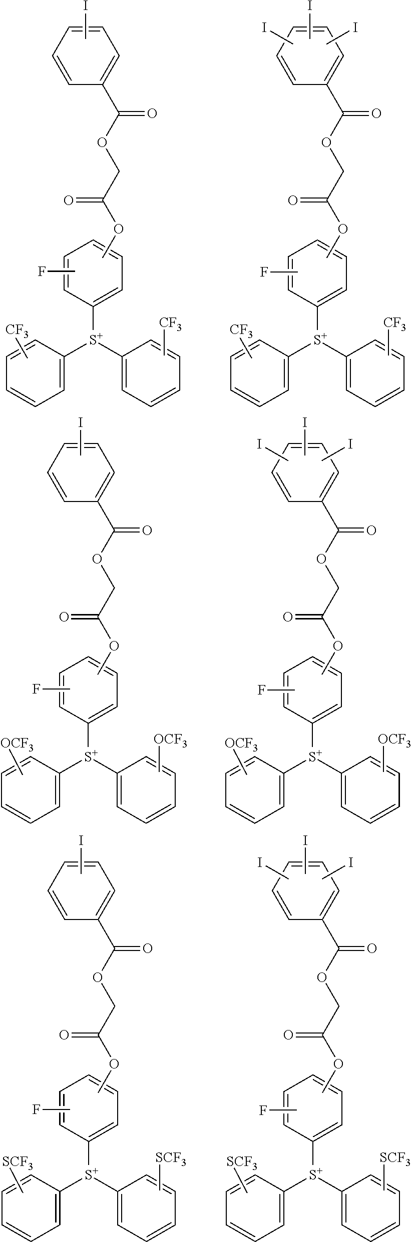

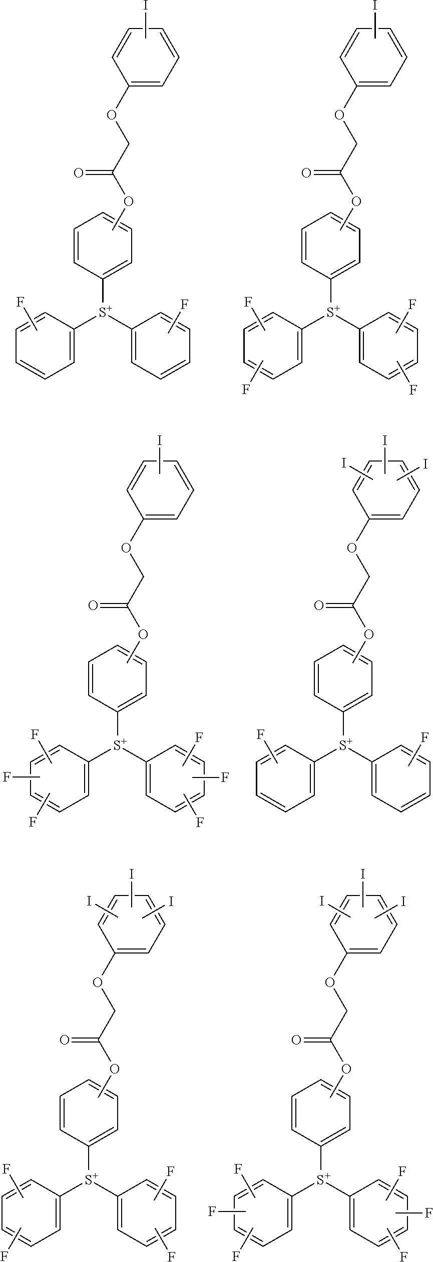

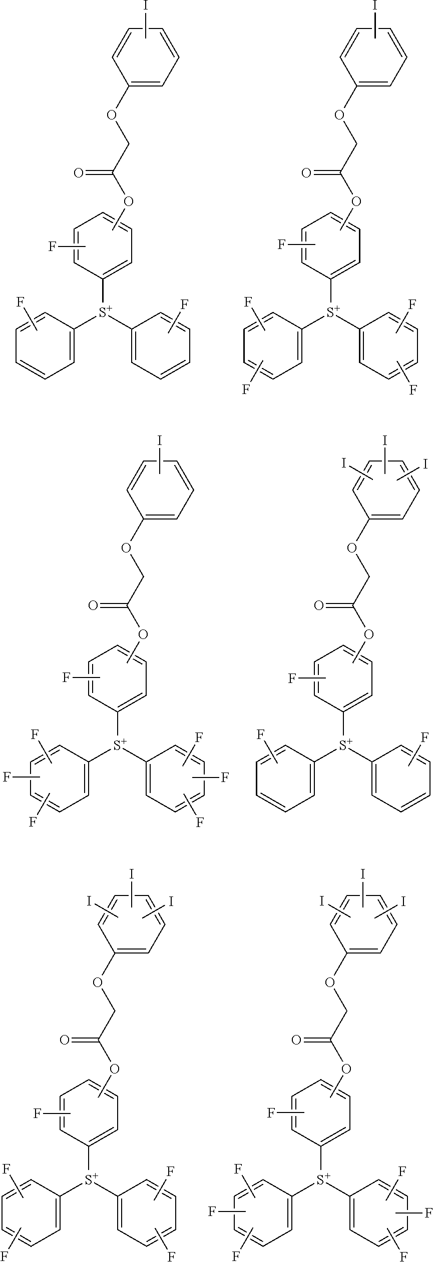

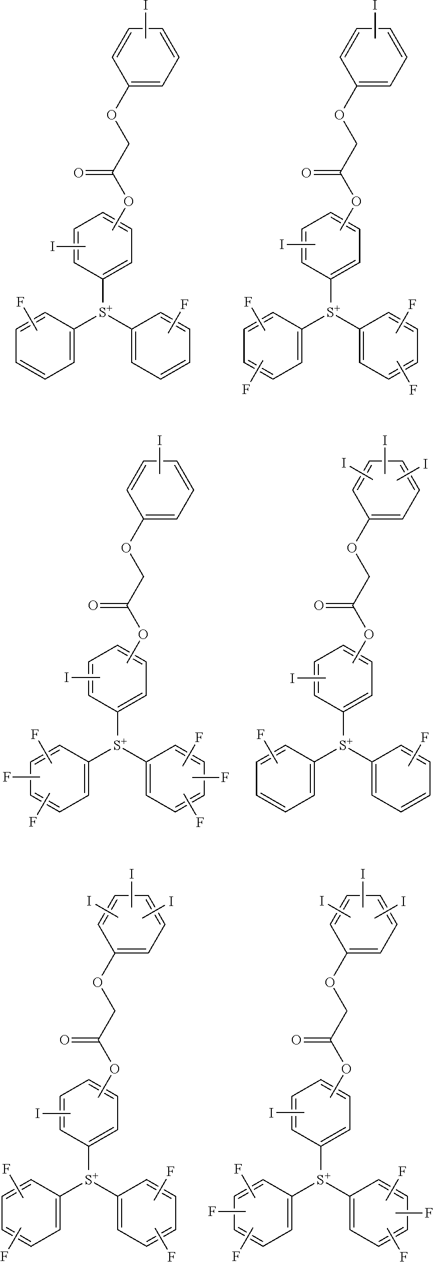

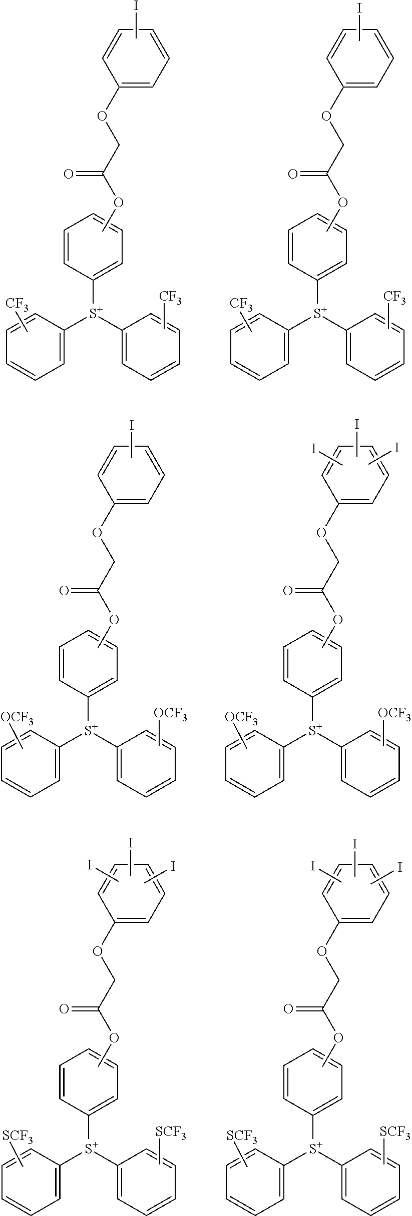

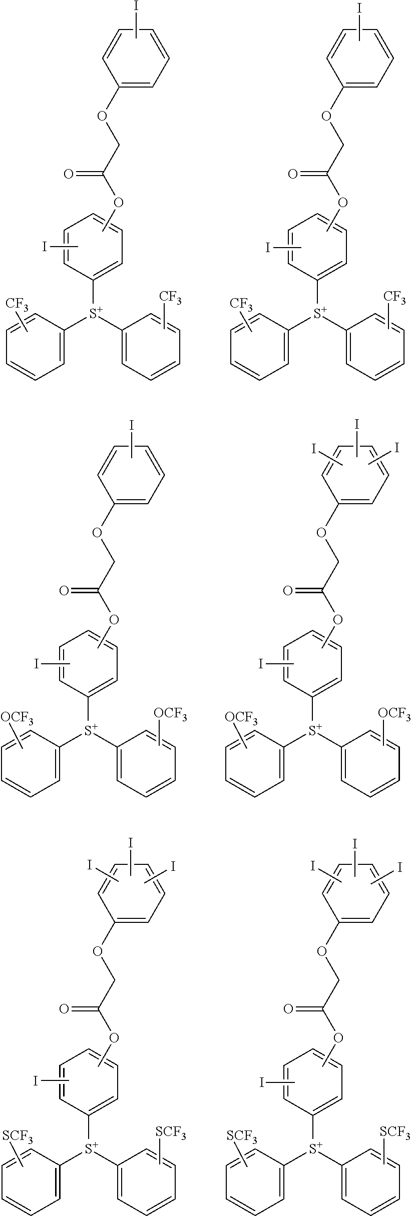

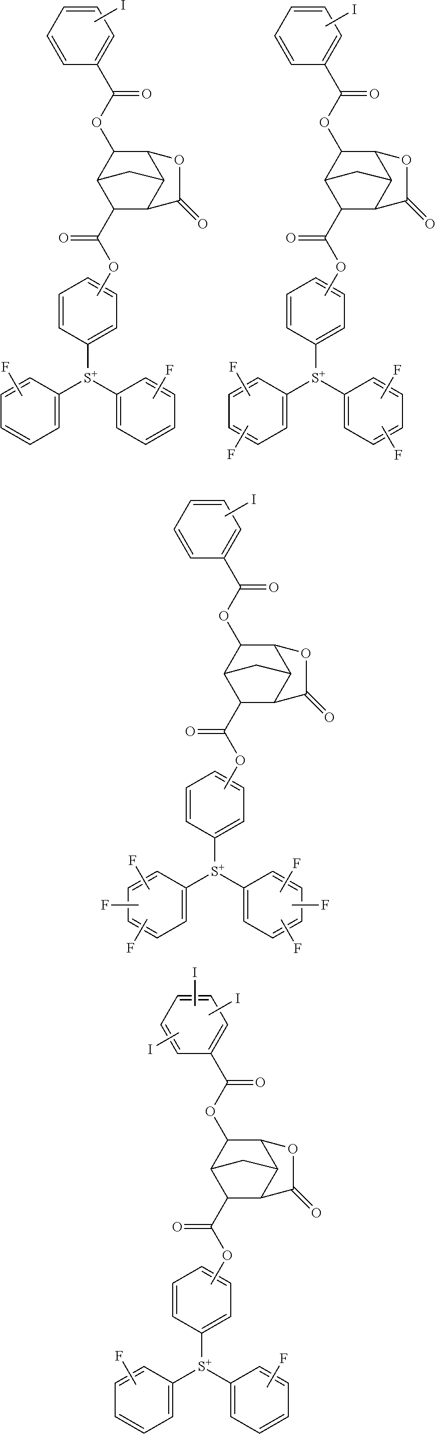

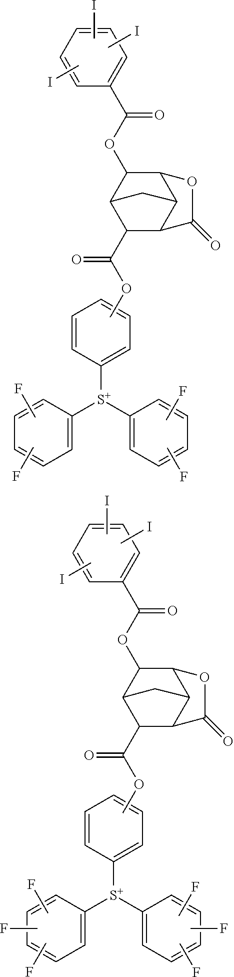

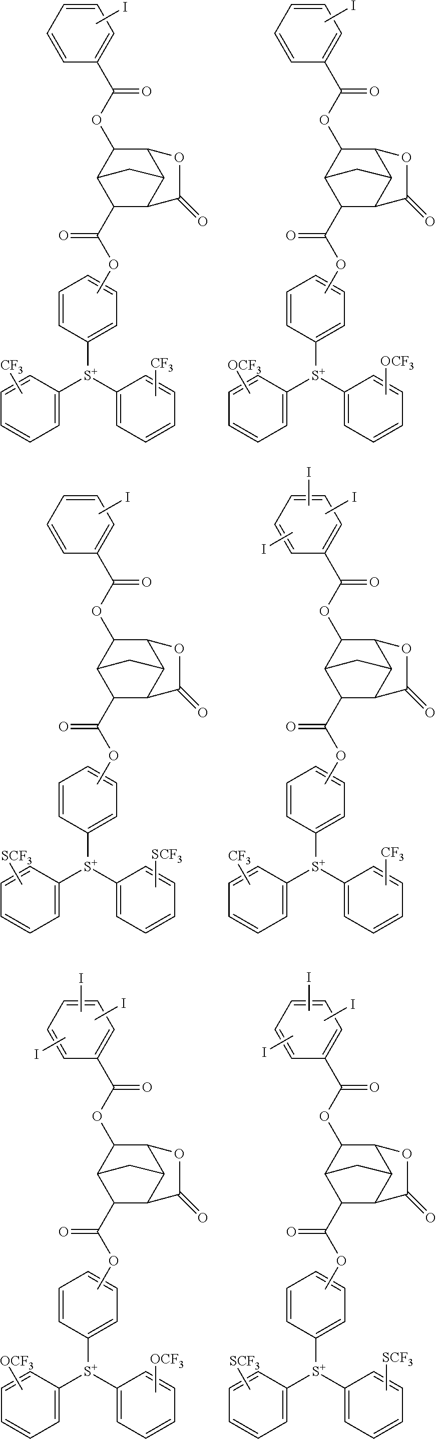

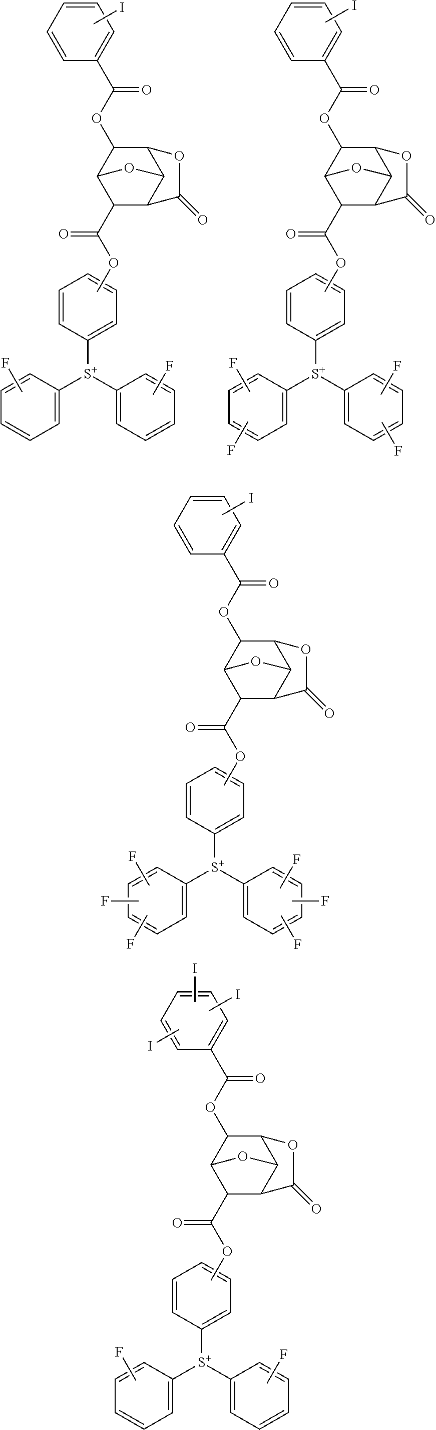

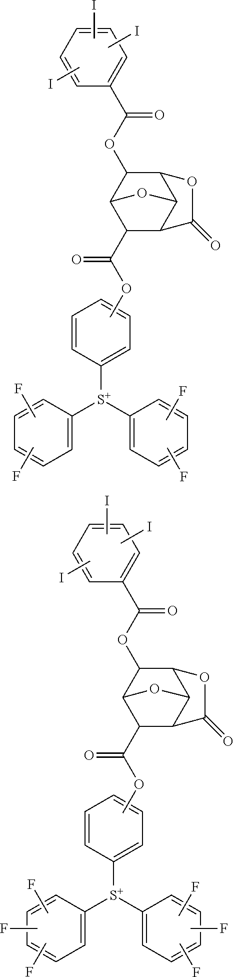

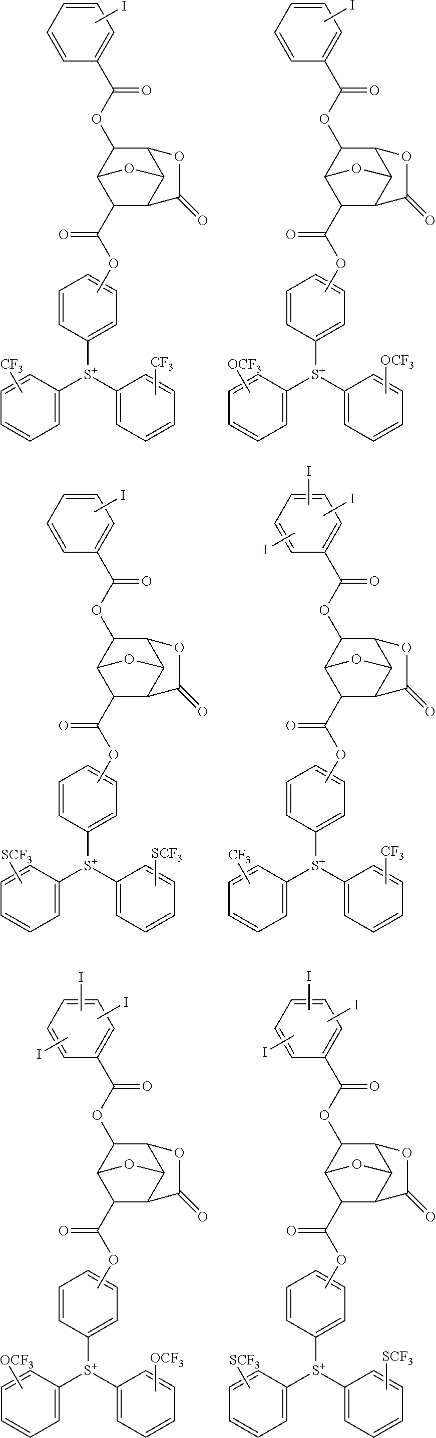

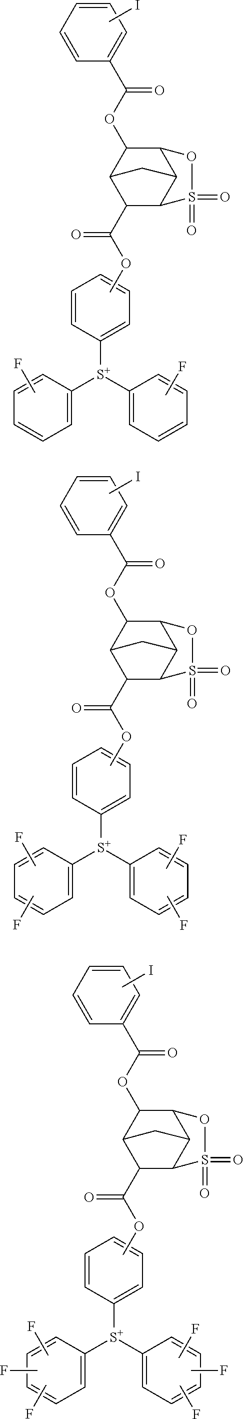

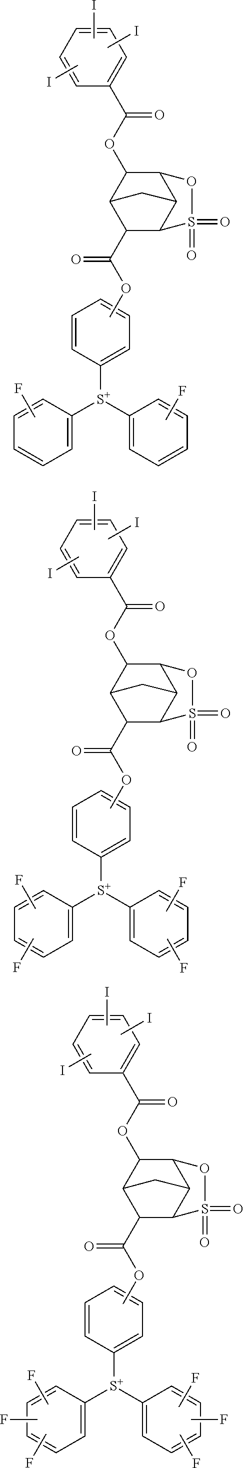

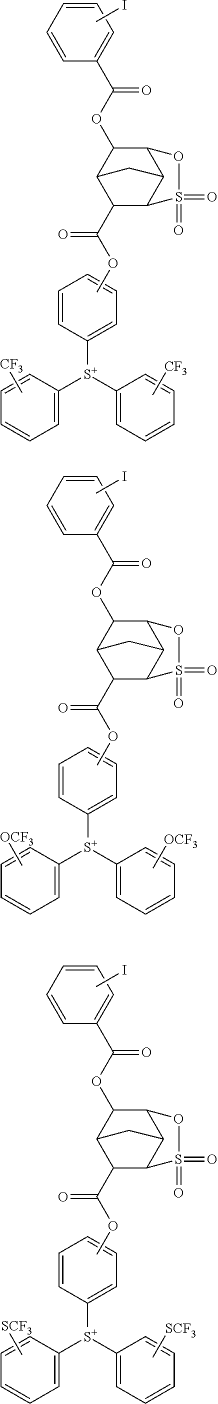

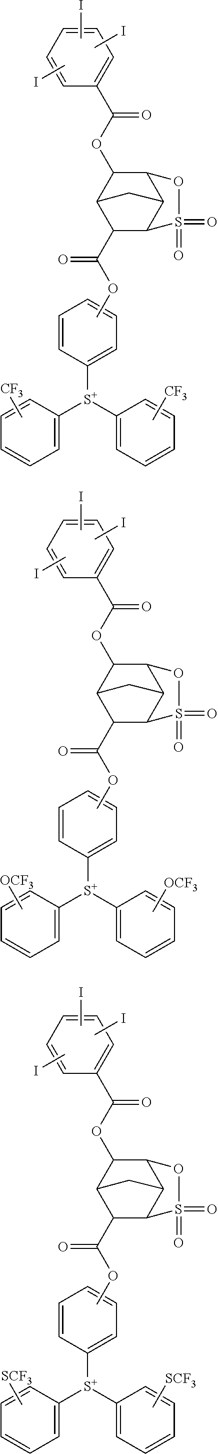

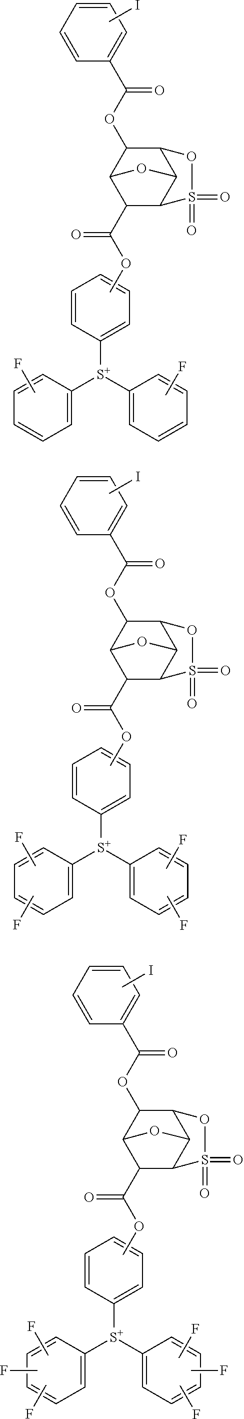

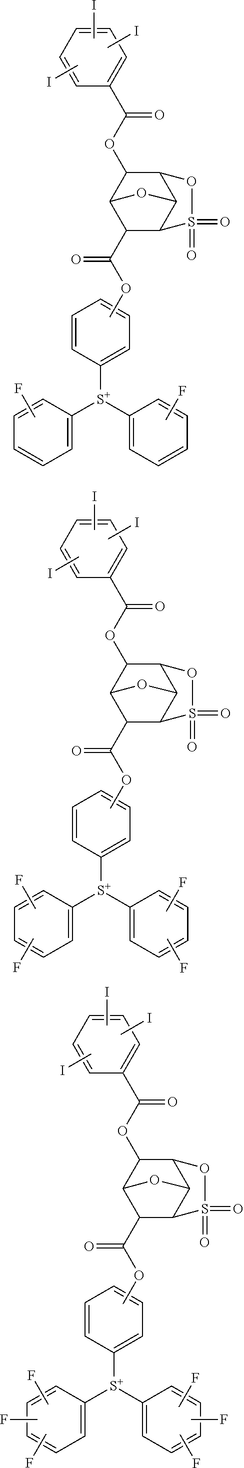

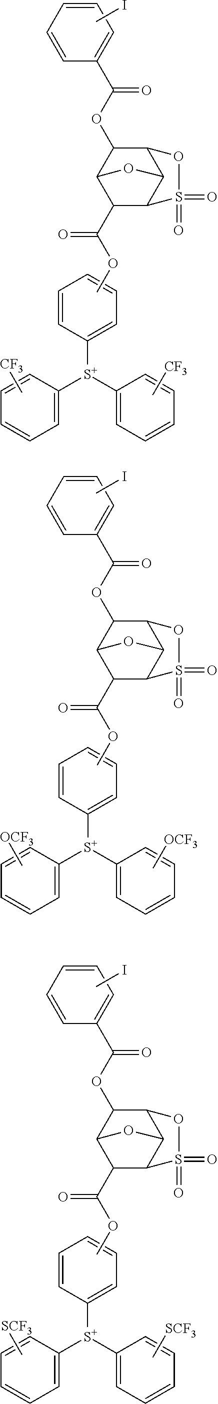

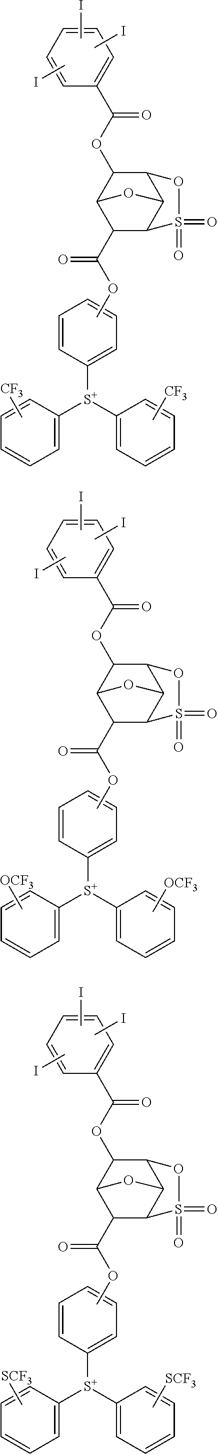

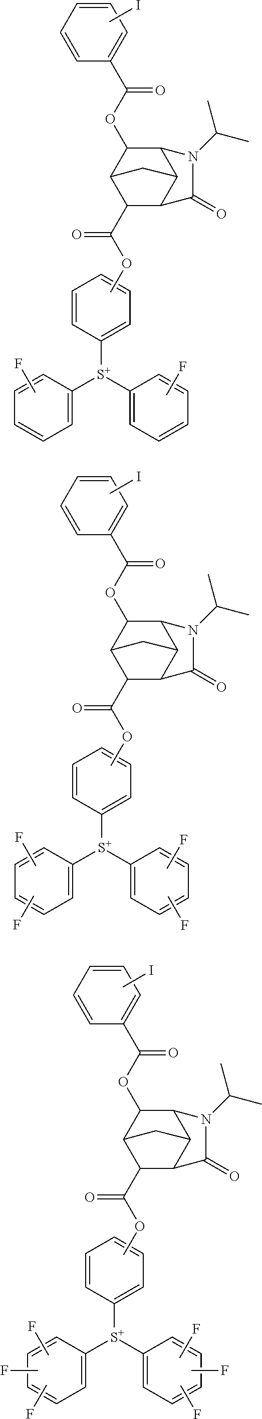

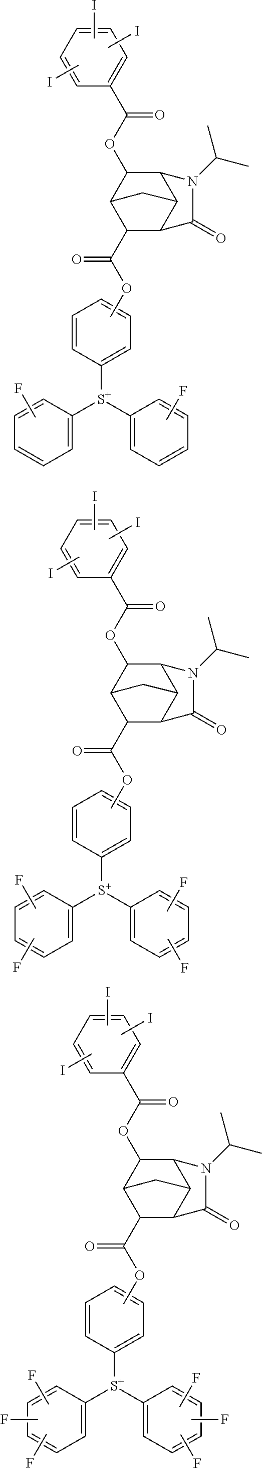

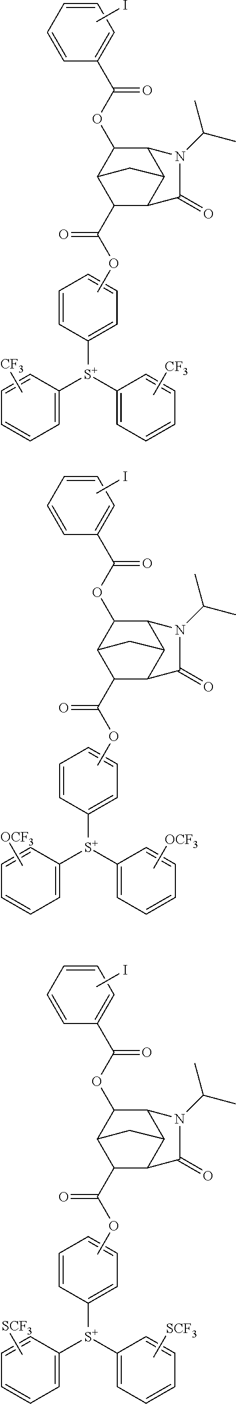

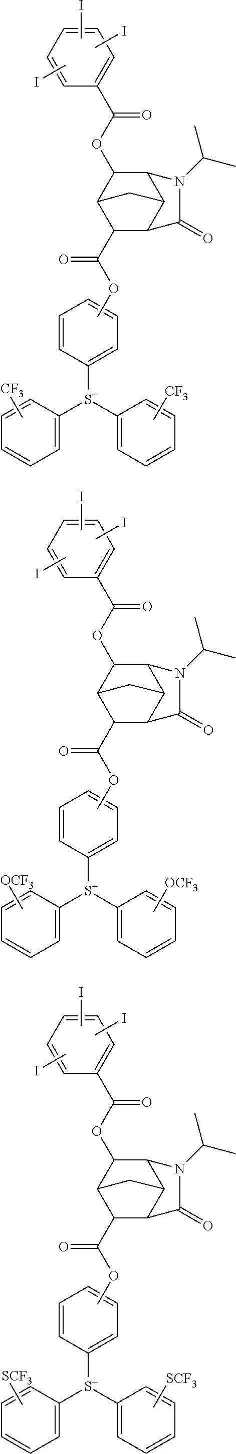

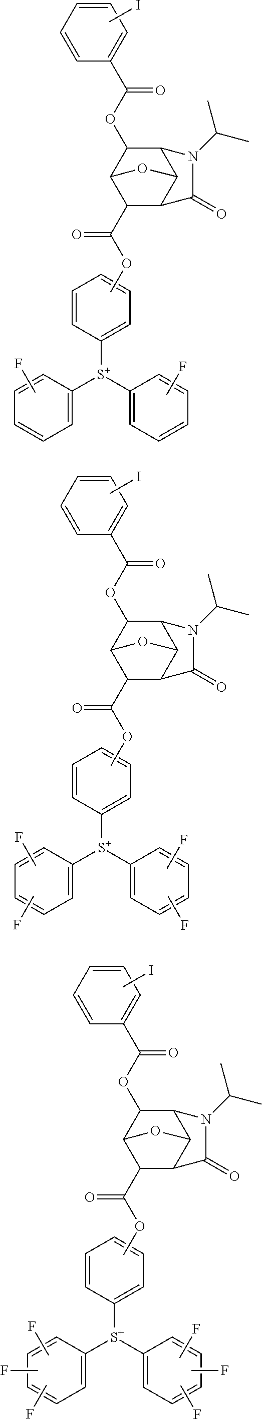

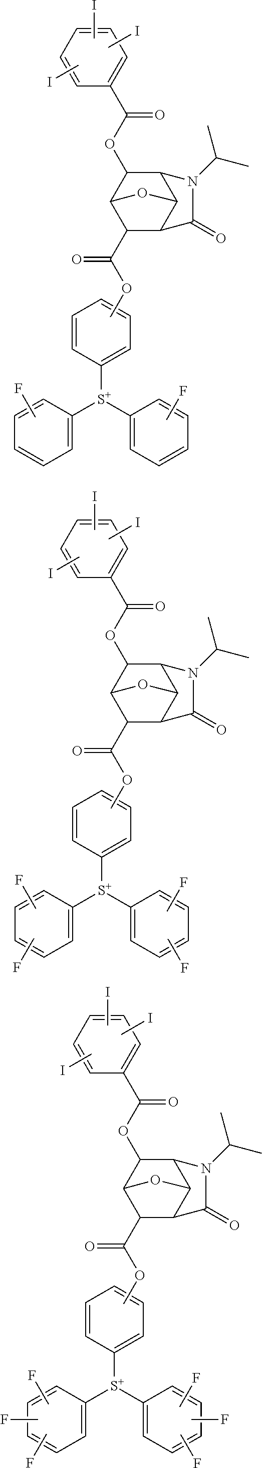

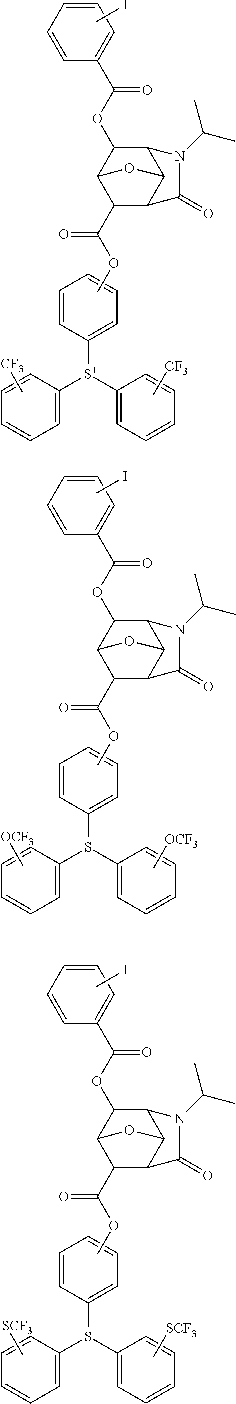

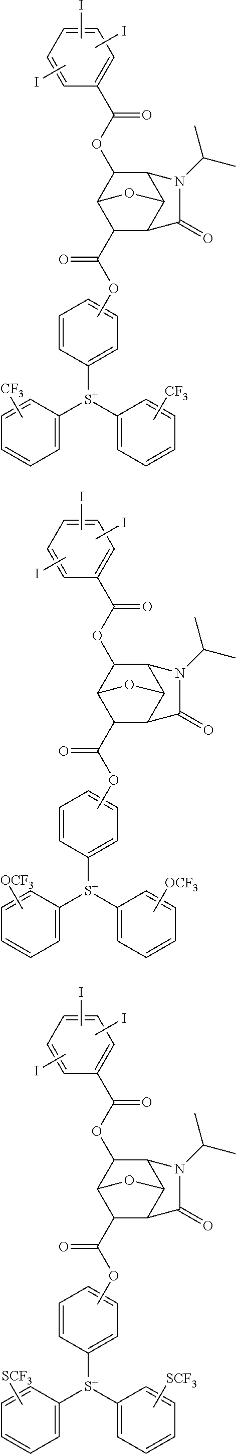

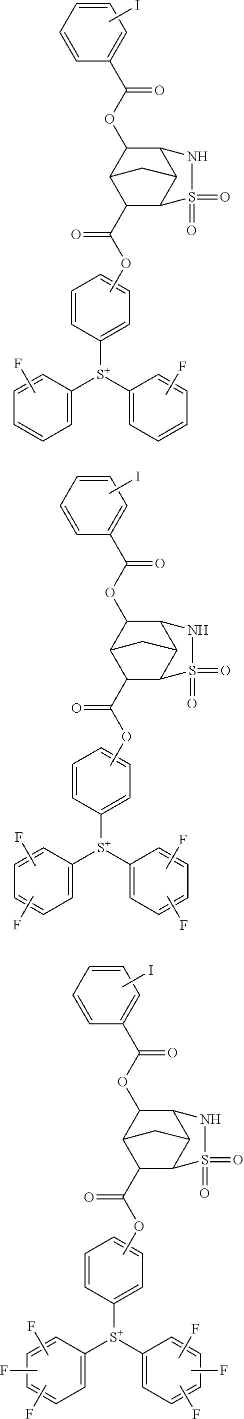

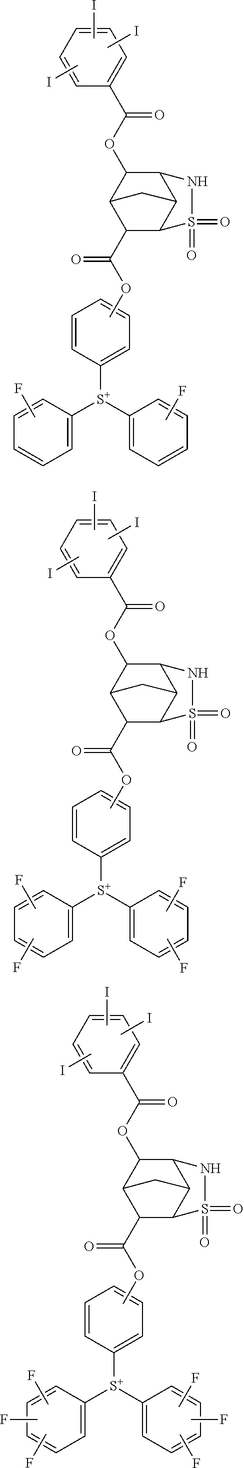

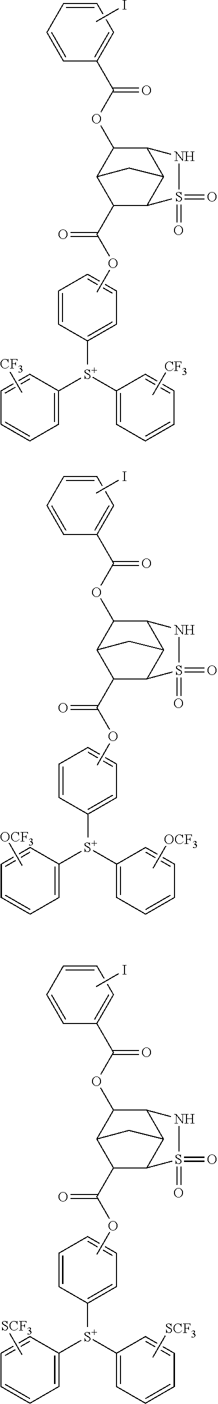

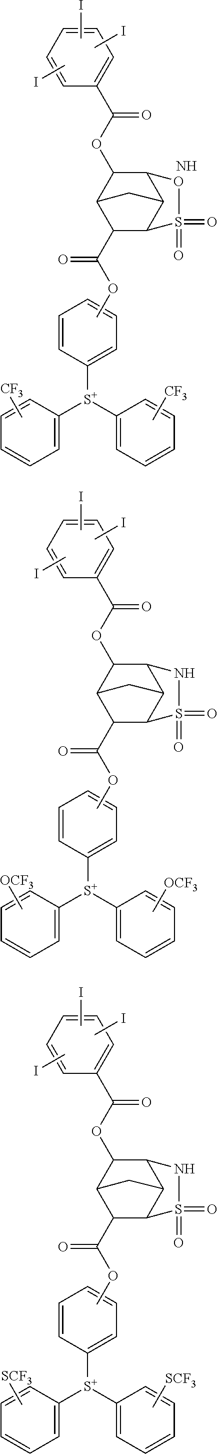

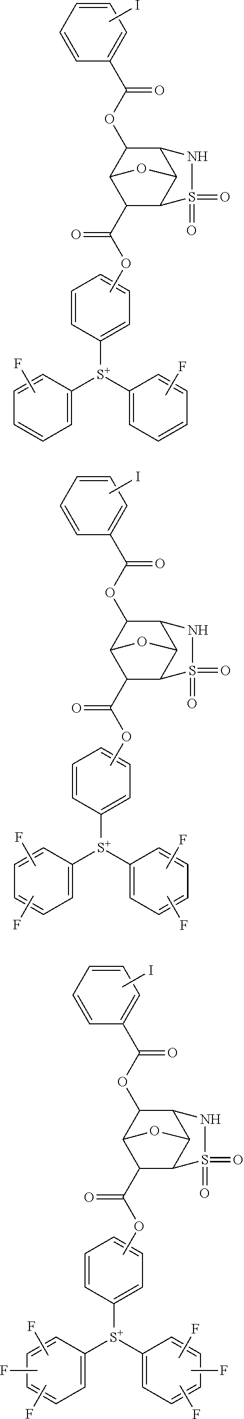

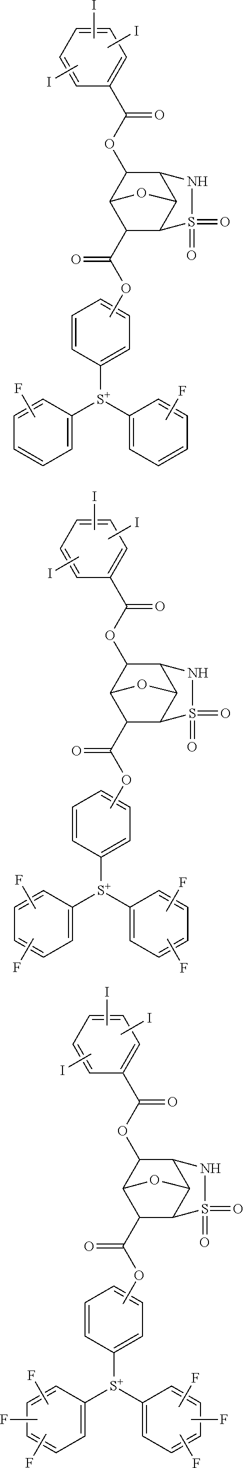

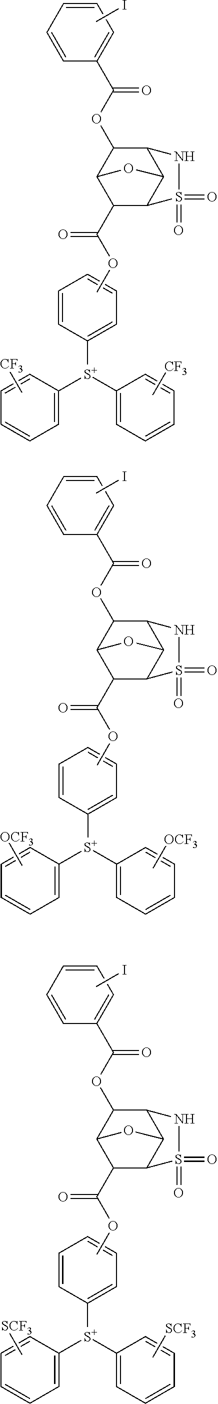

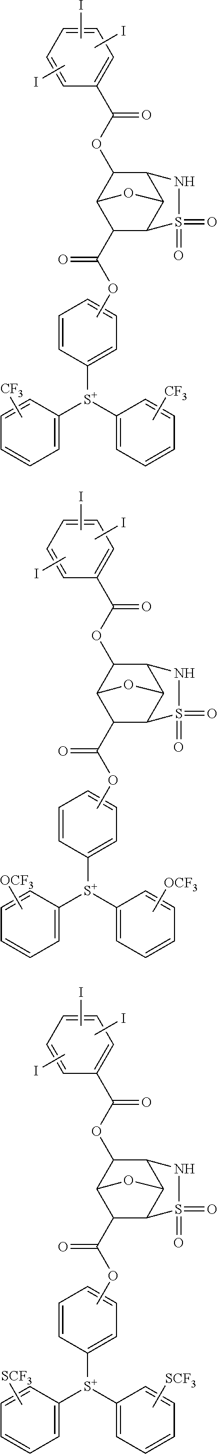

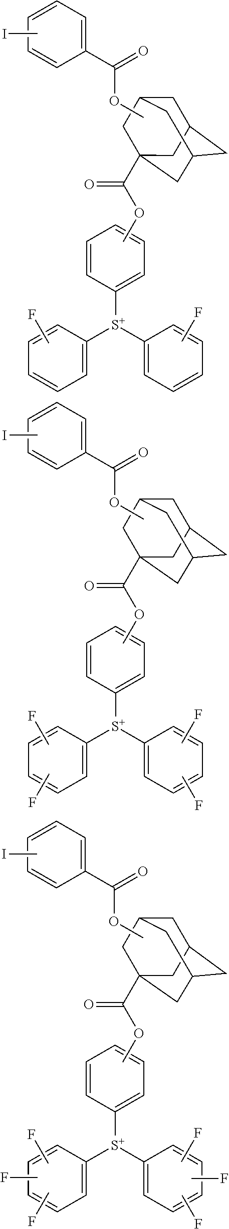

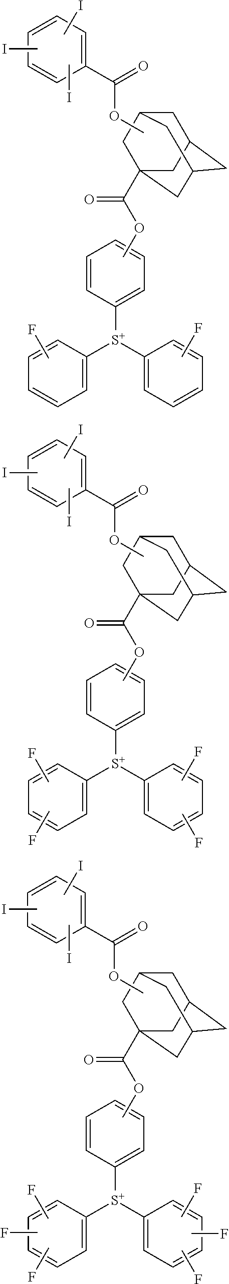

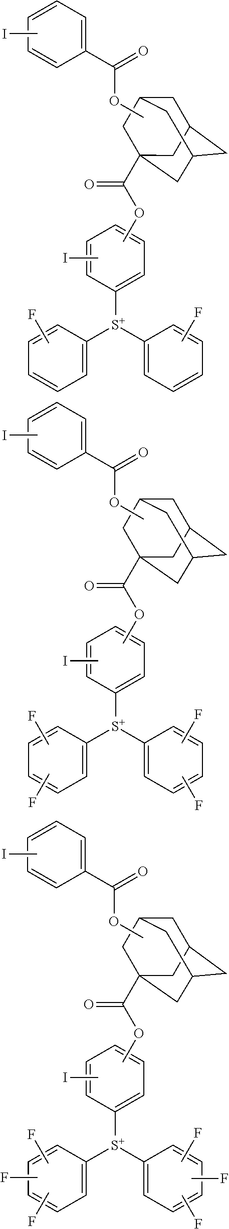

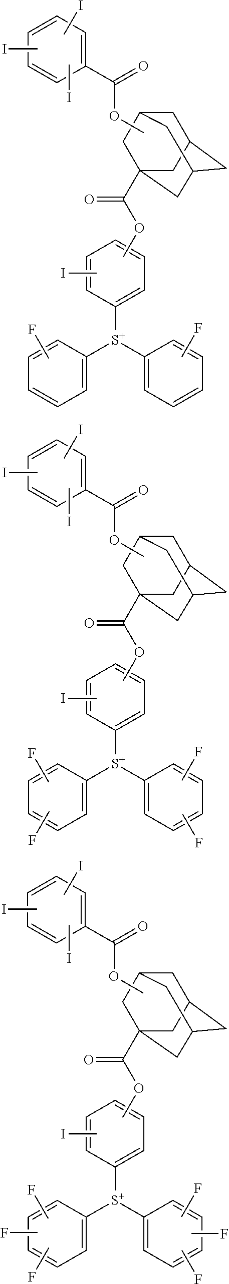

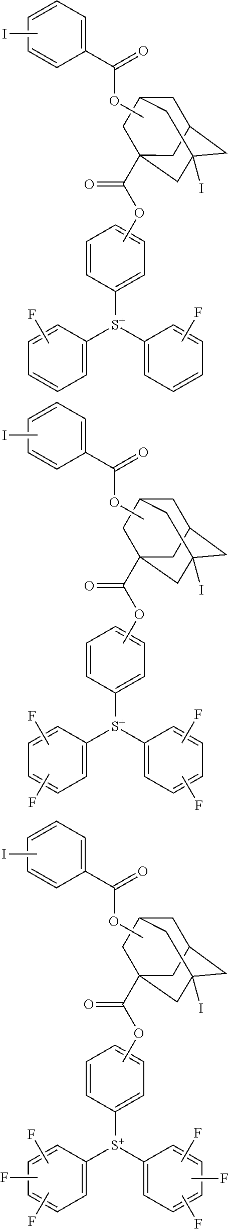

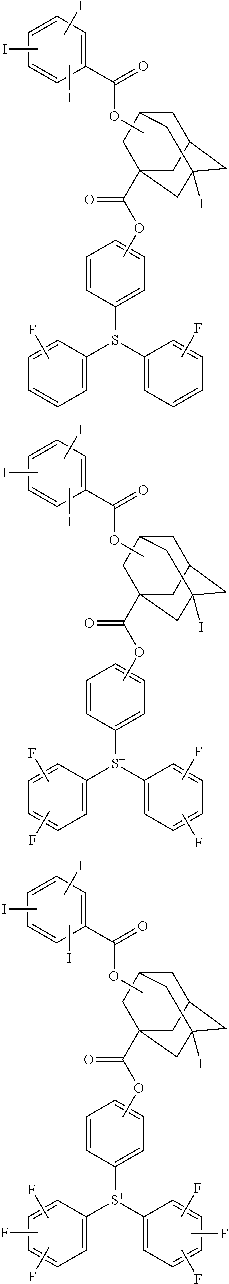

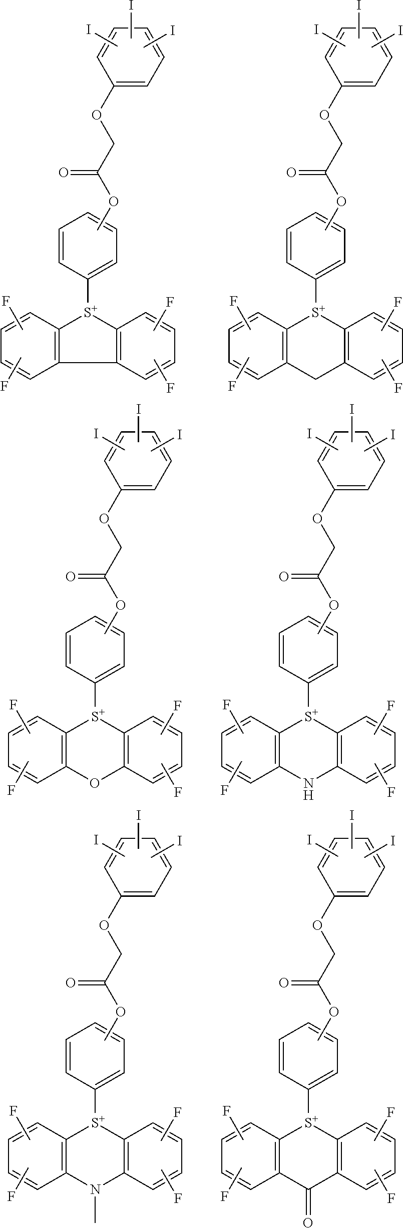

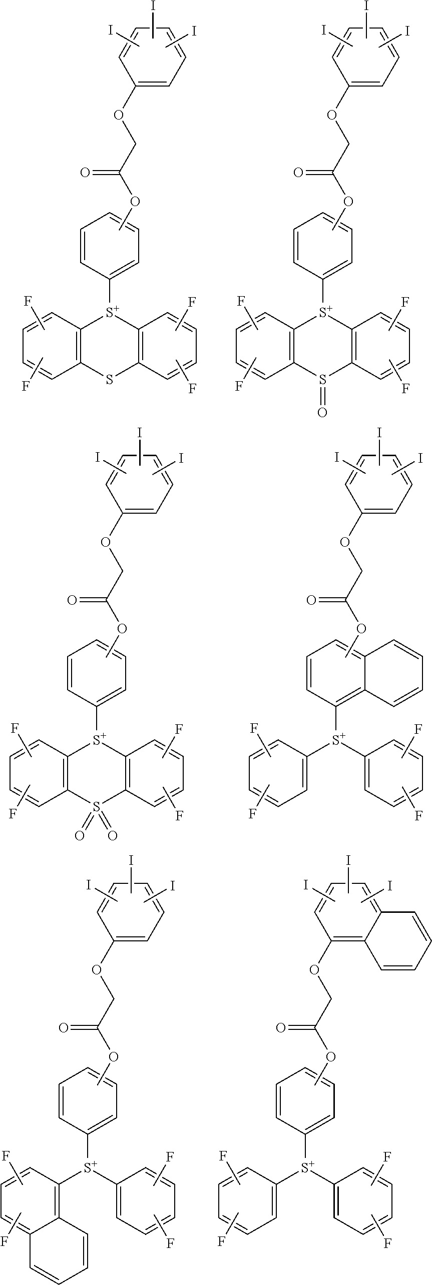

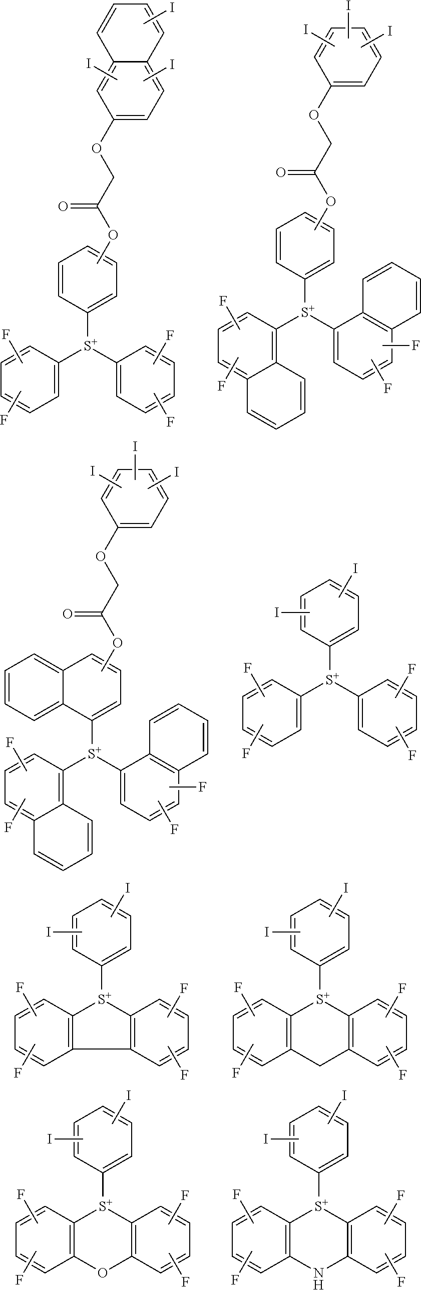

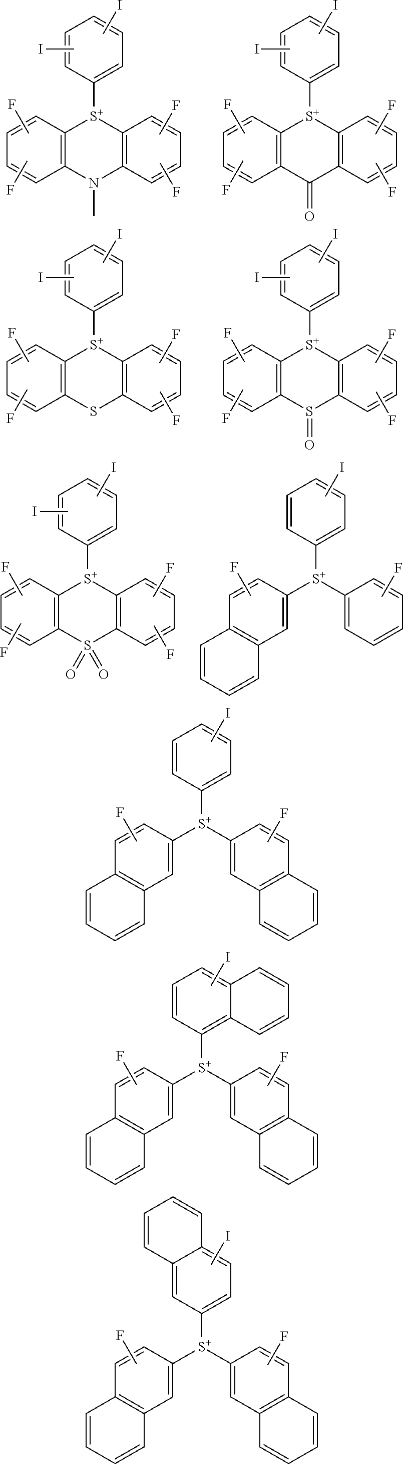

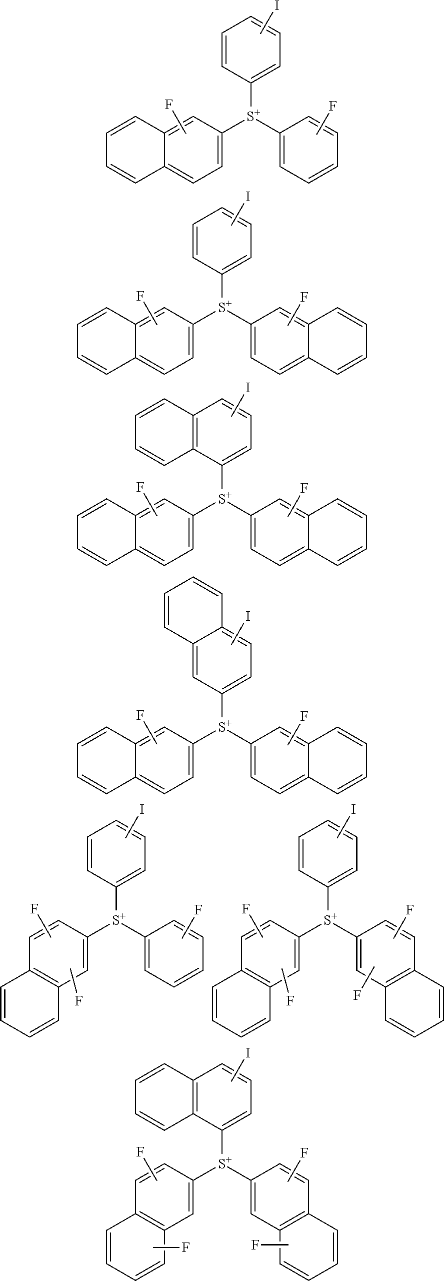

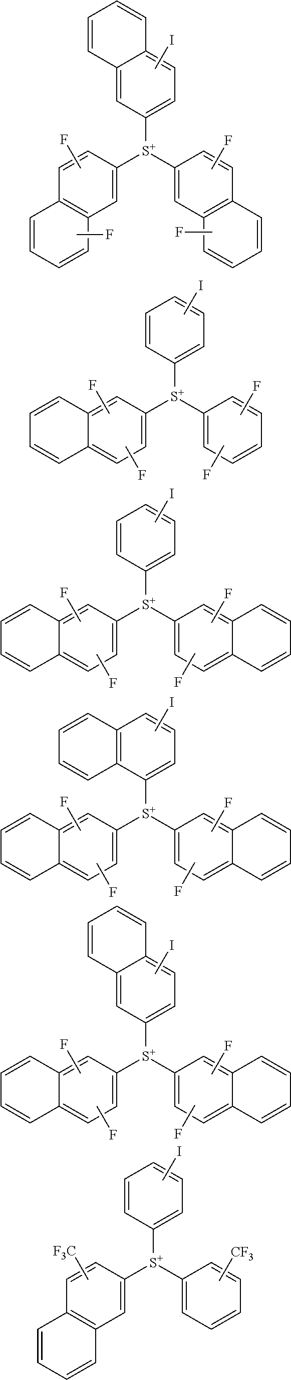

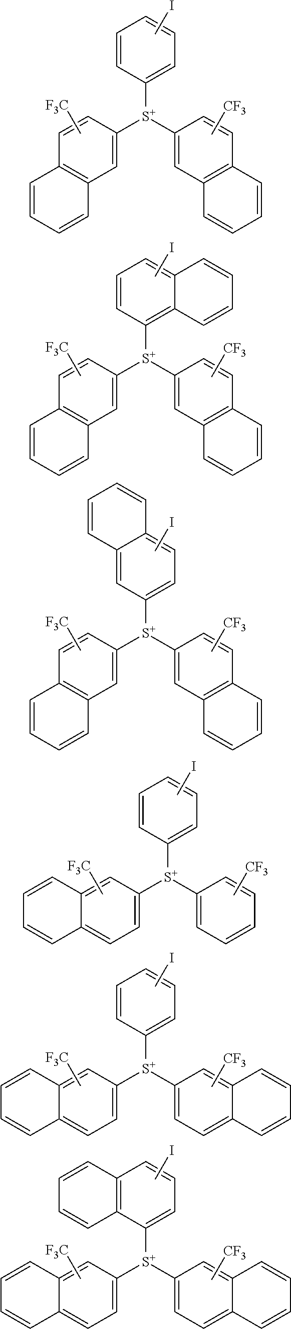

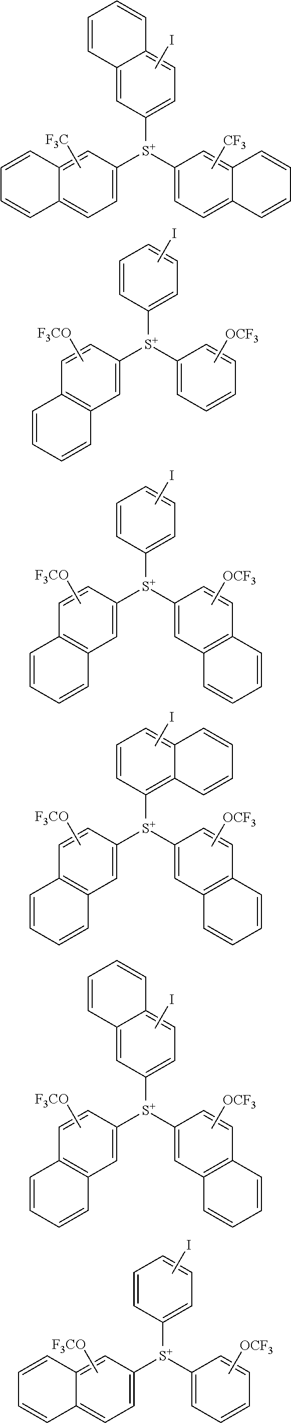

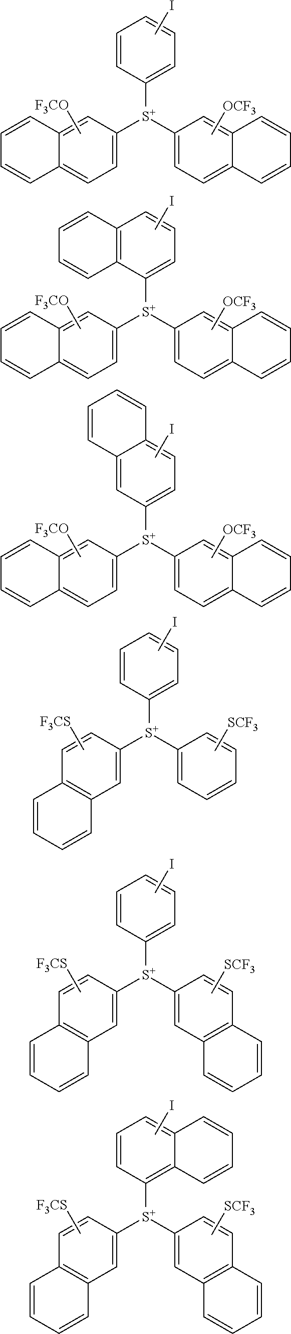

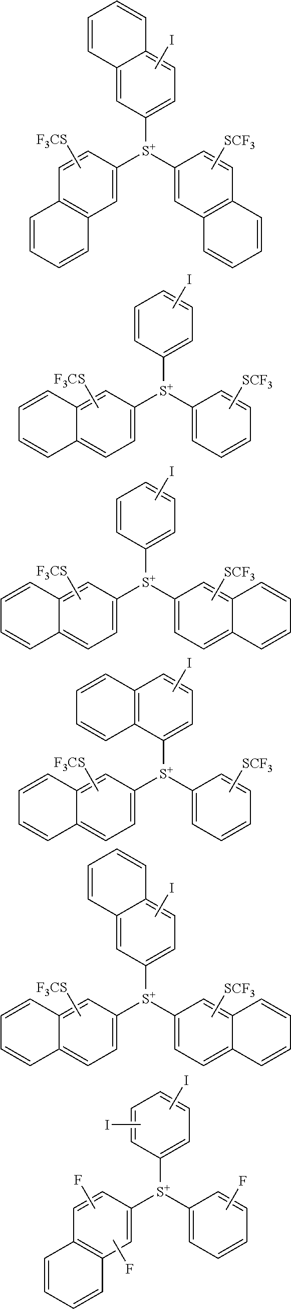

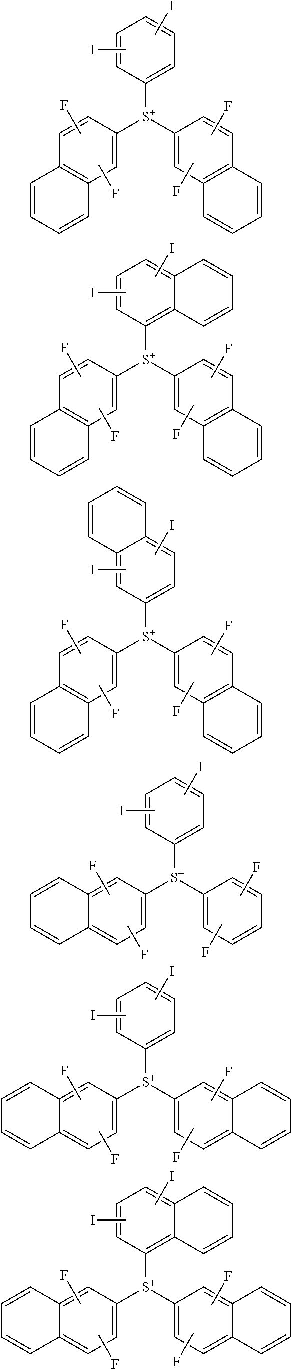

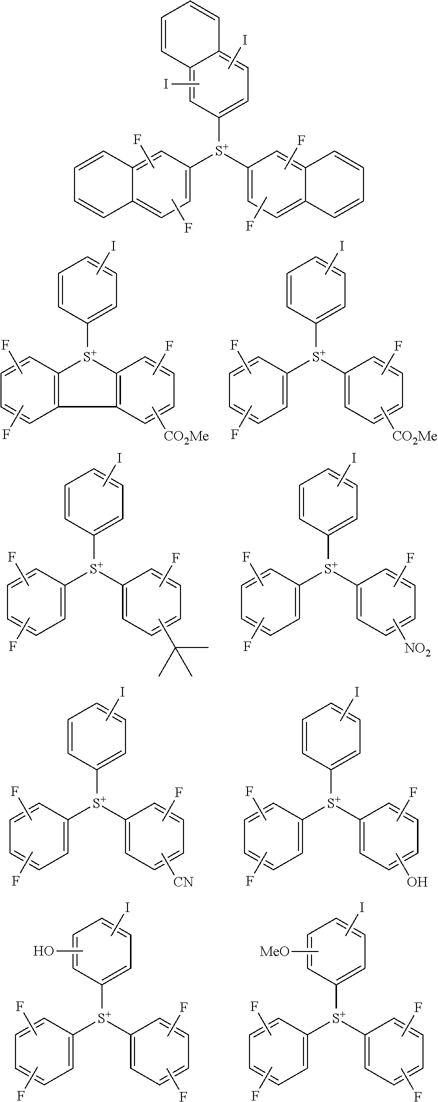

Specific examples of the iodonium cation include, but are not limited to, those described in paragraph [0181] of JP2024-000259A.

Specific examples of the ammonium cation include those represented by the following general formula (am-1).

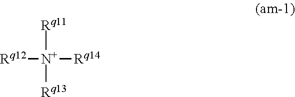

In the general formula (am-1), R^{q11} to R^{q14} each independently represent a hydrocarbyl group having 1 to 40 carbon atoms and optionally containing a heteroatom. Furthermore, R^{q11} and R^{q12} may be bonded to each other to form a ring together with the nitrogen atom to which they are bonded. Specific examples of the hydrocarbyl group include the same ones as those exemplified as the hydrocarbyl group represented by R¹ in the explanation of the general formula (1A).

Specific examples of the ammonium cation represented by the general formula (am-1) include, but are not limited to, the following.

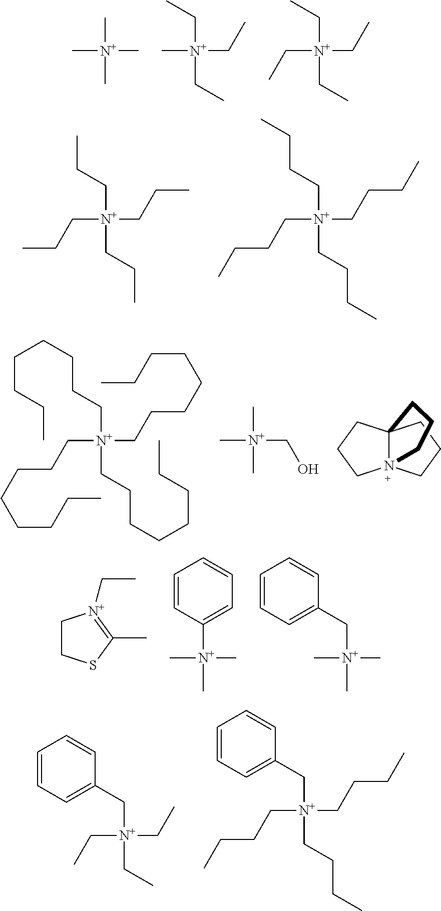

Specific structures of the repeating unit-c1 to c5 include any combination of the above-mentioned anions and cations.

Among the repeating unit-c1 to c5, the repeating unit-c2 to c5 are preferable from the viewpoint of controlling acid diffusion, the repeating unit-c2, c4, and c5 are more preferable from the viewpoint of the acid strength of the generated acid, and the repeating unit-c2 is further preferable from the viewpoint of solvent solubility.

The base polymer may further include a repeating unit having a structure in which a hydroxy group is protected by an acid-labile group (hereinafter also referred to as “repeating unit-d”). The repeating unit-d is not particularly limited as long as it has one or more structures in which a hydroxy group is protected and as long as the protecting group is decomposed by the action of an acid to generate a hydroxy group, but it is preferably one represented by the following general formula (d1).

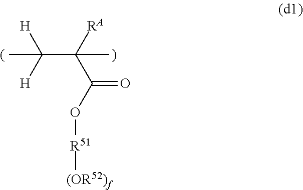

In the general formula (d1), R^{A} is as defined above. R⁵¹ represents an (f+1)-valent hydrocarbon group having 1 to 30 carbon atoms and optionally containing a heteroatom. R⁵² represents an acid-labile group. “f” represents an integer from 1 to 4.

In the general formula (d1), the acid-labile group represented by R⁵² may be any group that can be deprotected by the action of an acid to generate a hydroxy group. The structure of R⁵² is not particularly limited, but an acetal structure, a ketal structure, an alkoxycarbonyl group, an alkoxymethyl group represented by the following general formula (d2), etc. are preferable, and an alkoxymethyl group represented by the following general formula (d2) is particularly preferable.

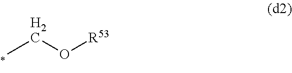

In the formula, * represents an attachment point; and R⁵³ represents a hydrocarbyl group having 1 to 15 carbon atoms.

Specific examples of the acid-labile group represented by R⁵², the alkoxymethyl group represented by the general formula (d2), and the repeating unit-d include the same ones as those exemplified in the explanation of the repeating unit-d described in JP2020-111564A.

The base polymer may further include a repeating unit-e derived from indene, benzofuran, benzothiophene, acenaphthylene, chromone, coumarin, norbornadiene, or derivatives thereof. Specific examples of monomers that provide the repeating unit-e include, but are not limited to, the following.

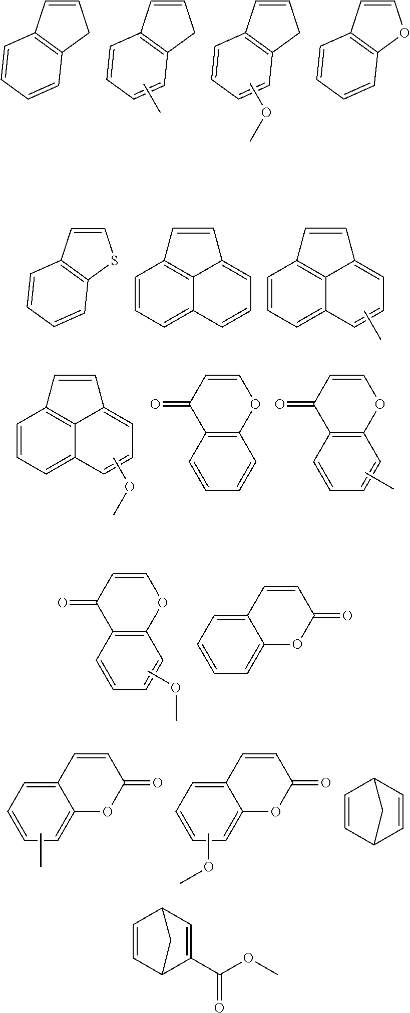

The base polymer may further include a repeating unit-f derived from indane, vinylpyridine, or vinylcarbazole.

In the base polymer, the content ratio of the repeating units-a1, a2, a3, b1, b2, c1 to c5, d, e, and f are preferably 0≤a1≤0.8, 0≤a2≤0.8, 0≤a3≤0.6, 0≤b1≤0.6, 0≤b2≤0.6, 0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3, and more preferably 0≤a1≤0.7, 0≤a2≤0.7, 0≤a3≤0.5, 0≤b1≤0.5, 0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3. However, 0≤a1+a2≤1.0 and a1+a2+a3+b1+b2+c1+c2+c3+c4+d+e+f≤1.0.

The weight-average molecular weight (Mw) of the base polymer is preferably 1,000 to 500,000, and more preferably 3,000 to 100,000. When Mw is within this range, sufficient etching resistance is obtained, and there is no risk of a decrease in resolution due to an inability to ensure a difference in dissolution rate before and after exposure. In the present invention, Mw is a polystyrene-equivalent measurement value obtained through gel permeation chromatography (GPC) using THF or N,N-dimethylformamide (DMF) as a solvent.

Furthermore, since the influence of Mw/Mn on the molecular weight distribution of the base polymer tends to become greater as the pattern rules become finer, it is preferable that Mw/Mn is a narrow distribution of 1.0 to 2.0 in order to obtain a resist composition that is suitable for use with fine pattern dimensions. Within the above range, there are few polymers with low or high molecular weights compared to Mw, and there is no risk of foreign matter being found on the pattern or the shape of the pattern deteriorating after exposure.

To synthesize the base polymer, for example, a monomer that provides the repeating unit described above may be polymerized in an organic solvent by adding a radical polymerization initiator and heating the resulting mixture.

Specific examples of the organic solvent used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL), etc. Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide, etc. The amount of these initiators added is preferably 0.01 to 25 mol % based on the total amount of monomers to be polymerized. The reaction temperature is preferably 50 to 150° C., and more preferably 60 to 100° C. The reaction time is preferably 2 to 24 hours, and from the viewpoint of production efficiency, more preferably 2 to 12 hours.

The polymerization initiator may be added to the monomer solution and then fed into the reaction vessel, or an initiator solution may be prepared separately from the monomer solution, and each of them may be fed independently into the reaction vessel. During the waiting time, radicals generated from the initiator may cause the polymerization reaction to proceed, resulting in the production of ultra-high molecular weight molecules; therefore, from the viewpoint of quality control, it is preferable to prepare and dropwise add the monomer solution and the initiator solution separately. The acid-labile group may be used as is after being introduced into the monomer, or may be protected or partially protected after polymerization. In addition, a known chain transfer agent such as dodecyl mercaptan or 2-mercaptoethanol may be used in combination to adjust the molecular weight. In this case, the amount of these chain transfer agents added is preferably 0.01 to 20 mol % based on the total amount of monomers to be polymerized.

In the case of monomers containing hydroxy groups, the hydroxy groups may be substituted with acetal groups such as ethoxyethoxy groups, which are easily deprotected by acids, during polymerization, and then deprotected with weak acid and water after polymerization; alternatively, the hydroxy groups may be substituted with acetyl groups, formyl groups, pivaloyl groups, etc., and then subjected to alkaline hydrolysis after polymerization.

When copolymerizing hydroxystyrene or hydroxyvinylnaphthalene, hydroxystyrene or hydroxyvinylnaphthalene may be polymerized by heating with other monomers in an organic solvent after adding a radical polymerization initiator, or it is possible to use acetoxystyrene or acetoxyvinylnaphthalene and, after polymerization, deprotect the acetoxy group by alkaline hydrolysis to produce polyhydroxystyrene or hydroxypolyvinylnaphthalene.

For the base for alkaline hydrolysis, ammonia water, triethylamine, etc. can be used. Furthermore, the reaction temperature is preferably −20 to 100° C., and more preferably 0 to 60° C. The reaction time is preferably 0.2 to 100 hours, and more preferably 0.5 to 20 hours.

The amount of each monomer in the monomer solution may be appropriately set so as to obtain the preferable content ratio of the repeating units described above.

For the base polymer obtained by the above-mentioned production method, a reaction solution obtained by a polymerization reaction may be used as the final product, or a powder obtained through a purification process such as a reprecipitation method in which a polymerization solution is added to a poor solvent to obtain a powder may be used as the final product; however, from the viewpoint of work efficiency and quality stabilization, it is preferable to use as the final product a base polymer solution obtained by dissolving the powder obtained through the purification process in a solvent.

Specific examples of the solvent to be used in this case, as described in paragraphs [0144] to [0145] of JP2008-111103A, include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling-point alcohol solvents such as diethylene glycol, propylene glycol, glycerin, 1,4-butanediol, and 1,3-butanediol; and mixed solvents thereof.

In the base polymer solution, the concentration of the base polymer is preferably 0.01 to 30% by mass, and more preferably 0.1 to 20% by mass.

The reaction solution and the base polymer solution are preferably filtered. Filtering can remove foreign matter and gels that may cause defects, and is effective for stabilizing quality.

The filter material used for the above-mentioned filtration may be a fluorocarbon-based, cellulose-based, nylon-based, polyester-based, or hydrocarbon-based material, but in the filtration step of the resist composition, a fluorocarbon-based filter known as Teflon (registered trademark), a hydrocarbon-based filter such as polyethylene or polypropylene, or a filter made of nylon is preferable. The pore size of the filter can be appropriately selected according to the desired degree of cleanliness, but is preferably 100 nm or less, and more preferably 20 nm or less. These filters may be used alone or in combination with a plurality of filters. The filtration method may involve passing the solution through the filter only once, but it is more preferable to circulate the solution to filter it a plurality of times. The filtration step can be performed in any order and any number of times in the production process of the base polymer, but it is preferable to filter the reaction solution after the polymerization reaction, the base polymer solution, or both.

The base polymer (B) may be used alone or in a combination of two or more different polymers with different composition ratios, Mw, and/or Mw/Mn. Furthermore, the base polymer (B) may contain, in addition to the above-mentioned base polymer, a hydrogenated product of a ring-opening metathesis polymer, and the one described in JP2003066612A can be used for this purpose.

In addition, the chemically amplified resist composition of the present invention preferably further contains at least one constituent selected from an organic solvent, a quencher, a photoacid generator other than the above-mentioned photoacid generator, and a surfactant. Each component is explained in detail below.

### [Organic Solvent (C)]

The chemically amplified resist composition of the present invention may contain an organic solvent as component (C). The organic solvent (C) is not particularly limited as long as it can dissolve each of the components described above and below. Specific examples of such organic solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and mixed solvents thereof, etc.

Among these organic solvents, 1-ethoxy-2-propanol, PGMEA, cyclohexanone, GBL, DAA, and mixed solvents thereof are preferable, as they have particularly excellent solubility for component (B), the base polymer.

In the chemically amplified resist composition of the present invention, the content of the organic solvent (C) is preferably 200 to 5,000 parts by mass, and more preferably 400 to 3,500 parts by mass, per 80 parts by mass of the base polymer (B). The organic solvent (C) may be used alone or in a combination of two or more types.

### [Quencher (D)]

The chemically amplified resist composition of the present invention may contain a quencher as component (D). Note that in the present invention, a quencher is a material that traps the acid generated by the photoacid generator in the chemically amplified resist composition, thereby preventing the acid from diffusing into unexposed areas, so that a desired pattern is formed.

The quencher (D) includes an onium salt represented by the following general formula (2) or (3).

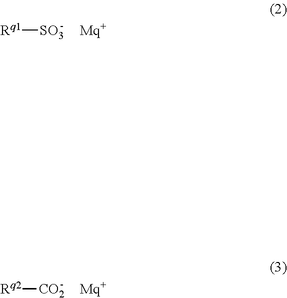

In the general formula (2), R^{q1} represents a hydrogen atom, or a hydrocarbyl group having 1 to 40 carbon atoms and optionally containing a heteroatom, except for those in which the hydrogen atom bonded to the carbon atom at the α-position of the sulfo group is substituted with a fluorine atom or a fluoroalkyl group. In the general formula (3), R^{q2} represents a hydrogen atom or a hydrocarbyl group having 1 to 40 carbon atoms and optionally containing a heteroatom.

Specific examples of the hydrocarbyl group having 1 to 40 carbon atoms represented by R^{q1} include alkyl groups having 1 to 40 carbon atoms, such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl groups; cyclic saturated hydrocarbyl groups having 3 to 40 carbon atoms, such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}] decyl, and adamantyl groups; and aryl groups with 6 to 40 carbon atoms, such as phenyl, naphthyl, and anthracenyl groups. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the hydrocarbyl group represented by R^{q2} include, in addition to the substituents exemplified as specific examples of R^{q1}, fluorinated saturated hydrocarbyl groups such as a trifluoromethyl group and a trifluoroethyl group; and fluorinated aryl groups such as a pentafluorophenyl group and a 4-trifluoromethylphenyl group.

Specific examples of the anion of the onium salt represented by the general formula (2) include, but are not limited to, the following.

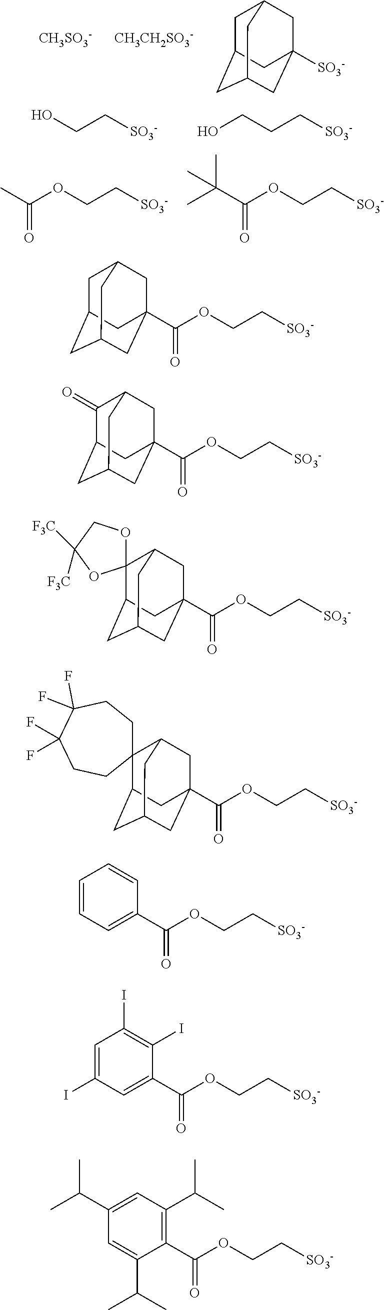

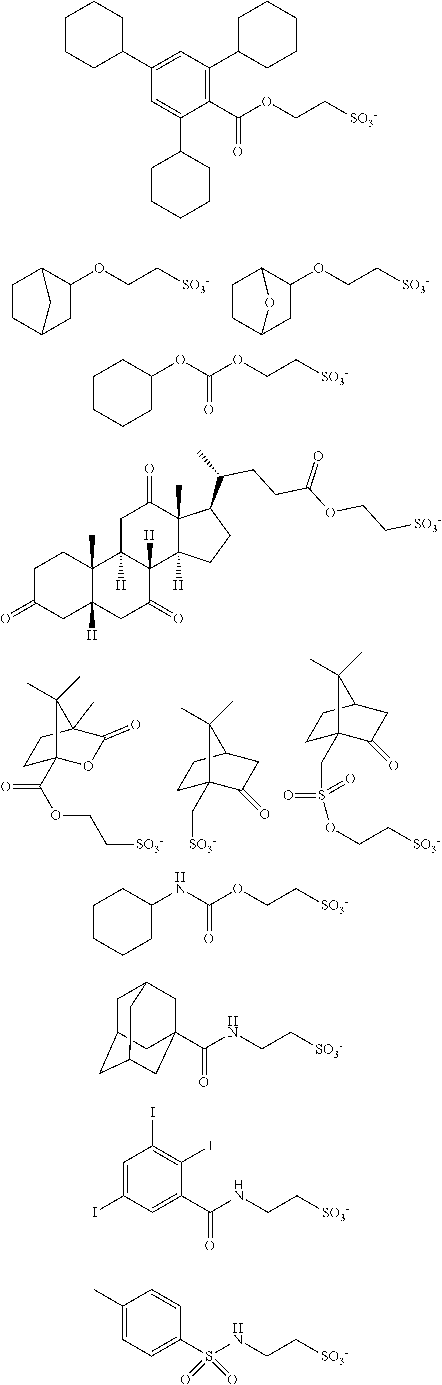

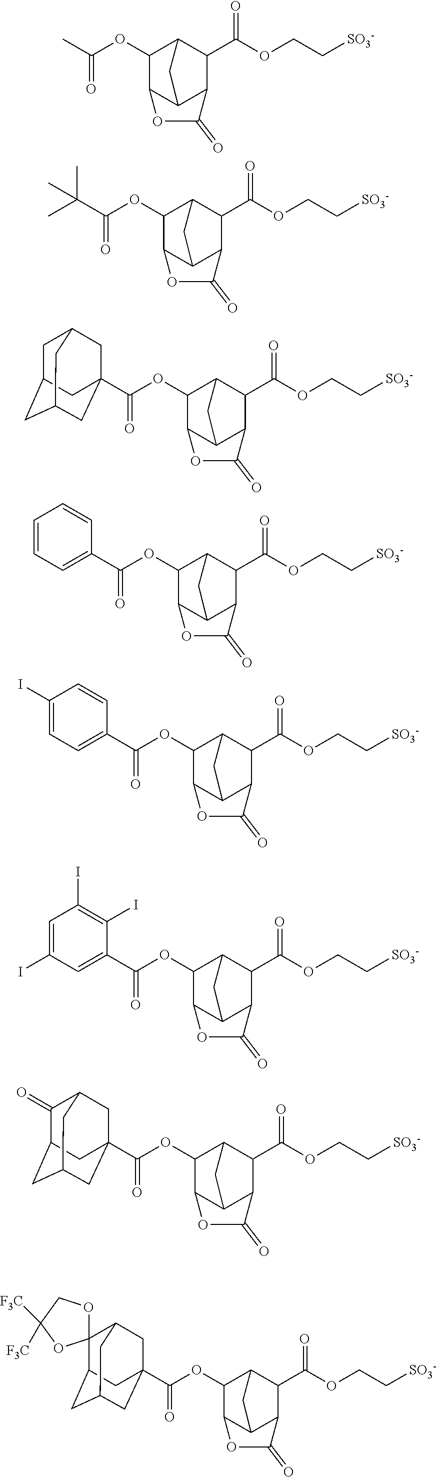

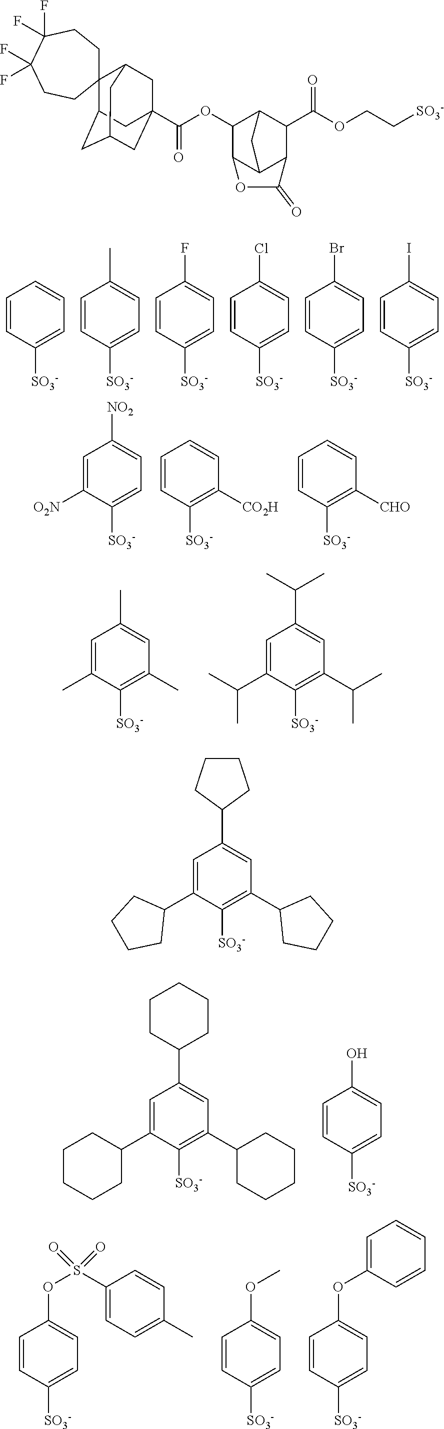

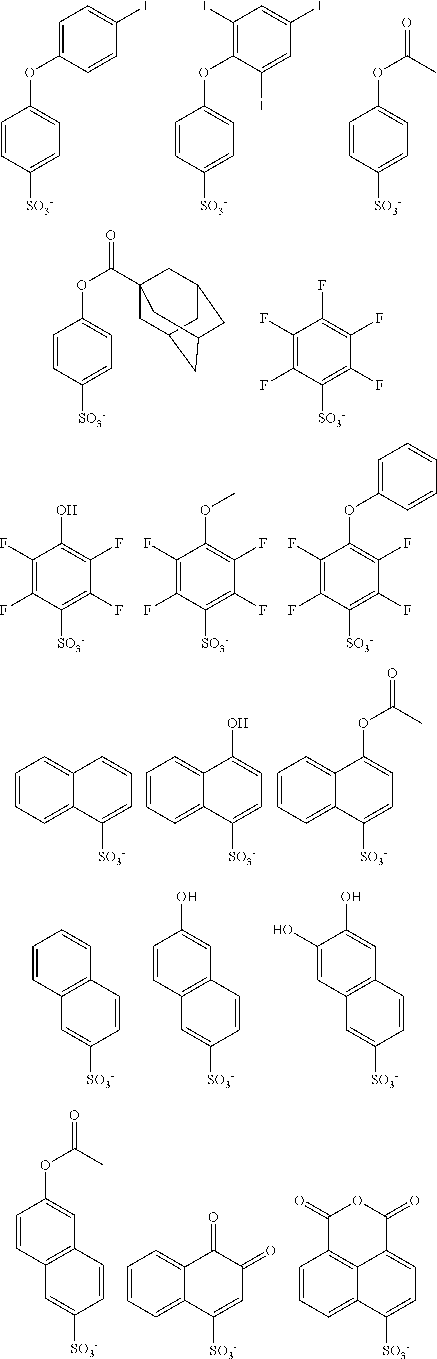

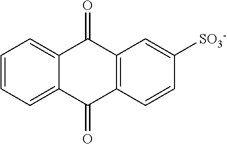

Specific examples of the anion of the onium salt represented by the general formula (3) include, but are not limited to, the following.

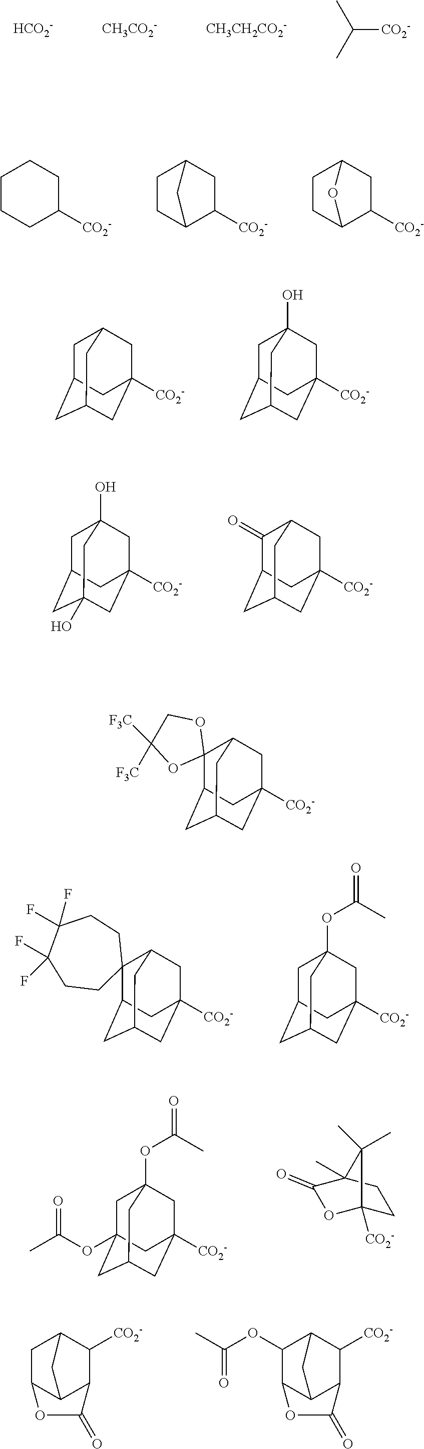

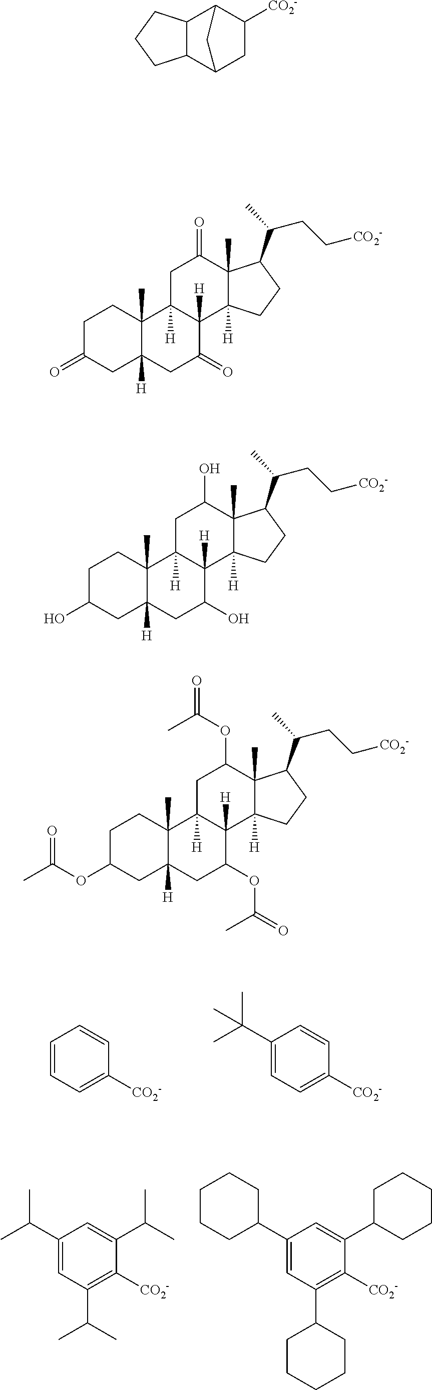

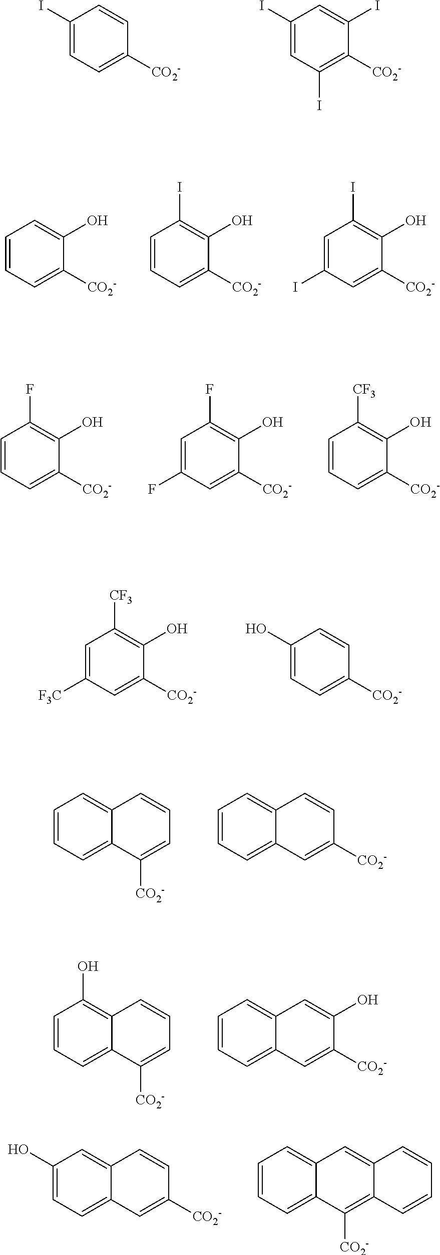

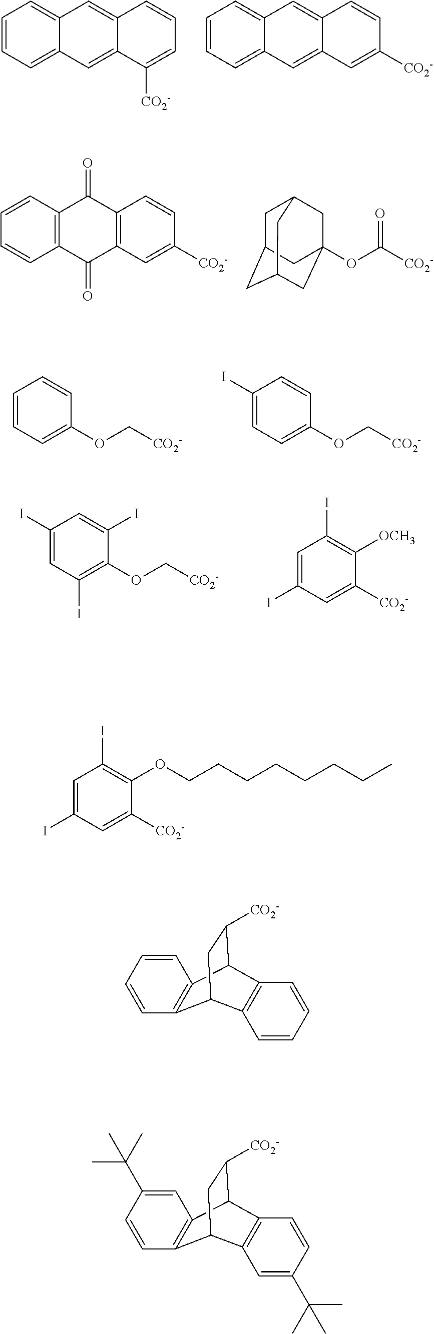

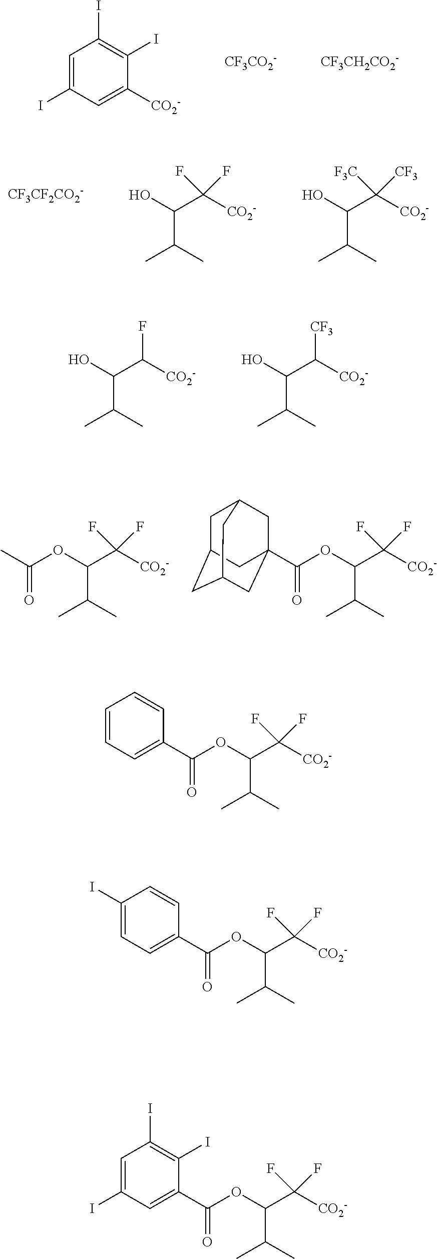

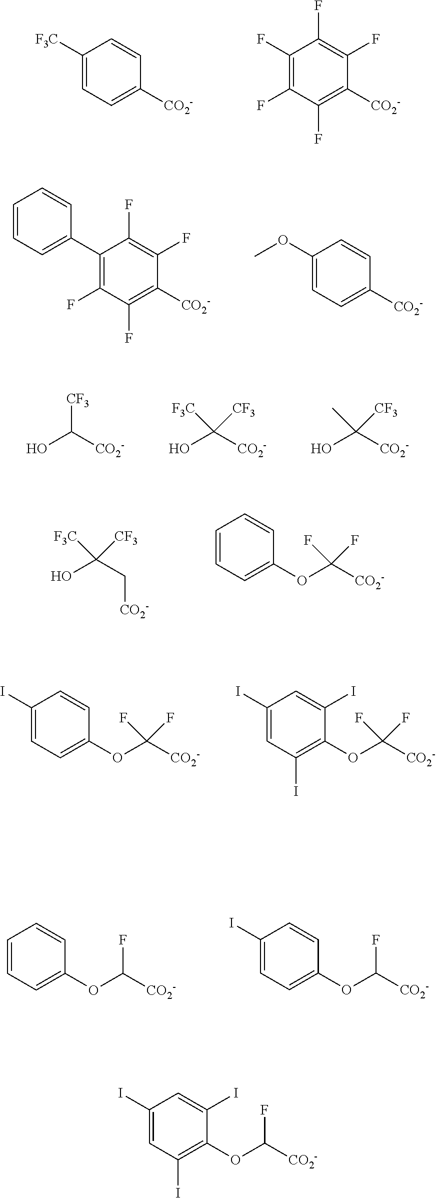

In the general formulae (2) and (3), Mq⁺ represents an onium cation. Examples of the onium cation include a sulfonium cation, an iodonium cation, and an ammonium cation. Specific examples of the sulfonium cation include those exemplified as specific examples of the sulfonium cation in the general formula (1B), those described in paragraphs [0102] to [0125] of JP2024-003744A, those described in paragraphs [0044] to [0049] of WO2024/128017A1, those described in paragraphs [0035] to [0046] of JP7491173B2, and those exemplified as specific examples of the sulfonium cation in the general formula (d3), etc., but are not limited thereto. Specific examples of the iodonium cation include, but are not limited to, those described in paragraph [0181] of JP2024-000259A. Specific examples of the ammonium cation include, but are not limited to, those represented by the general formula (am-1).

Specific examples of the onium salt represented by the general formula (2) or (3) include any combination of the above-mentioned anions and cations. These onium salts can be easily prepared by ion exchange reactions using known organic chemical methods. For the ion exchange reactions, for example, JP2007-145797A can be referred to.

The onium salt represented by the general formula (2) or (3) acts as a quencher in the chemically amplified resist composition of the present invention. This is because each counter anion of the onium salt is the conjugate base of a weak acid. The term “weak acid” used herein means an acidity that is not capable of deprotecting the acid-labile group of the acid-labile group-containing unit used in the base polymer. The onium salt represented by the general formula (2) or (3) functions as a quencher when used in combination with an onium salt-type photoacid generator having, as a counter anion, a conjugate base of a strong acid such as a sulfonic acid whose α-position is fluorinated. That is, when an onium salt that generates a strong acid such as a sulfonic acid whose α-position is fluorinated is mixed with an onium salt that generates a weak acid such as a non-fluorinated sulfonic acid or carboxylic acid, when the strong acid generated from the photoacid generator by irradiation with high-energy beams collides with an onium salt having an unreacted weak acid anion, the weak acid is released by salt exchange and an onium salt having a strong acid anion is generated. In this process, the strong acid is exchanged for a weaker acid with lower catalytic ability, and the acid becomes deactivated, allowing control of acid diffusion.

Furthermore, for the quencher (D), the following can also be used: onium salts having a sulfonium cation and a phenoxide anion moiety in the same molecule, as described in JP6848776B2, onium salts having a sulfonium cation and a carboxylate anion moiety in the same molecule, as described in JP6583136B2 and JP2020-200311A, and onium salts having an iodonium cation and a carboxylate anion moiety in the same molecule, as described in JP6274755B2.

Here, when the photoacid generator that generates a strong acid is an onium salt, the strong acid generated by irradiation with high-energy beams can be exchanged for a weak acid as described above; however, on the other hand, the weak acid generated by irradiation with high-energy beams is thought to collide with the unreacted onium salt that generates the strong acid, making it difficult to carry out salt exchange. This is due to the phenomenon that the onium cation tends to form an ion pair with the anion of a stronger acid.

When the chemically amplified resist composition of the present invention contains an onium salt represented by general formula (2) or (3) as the quencher (D), the content thereof is preferably 0.1 to 20 parts by mass, and more preferably 0.1 to 10 parts by mass, per 80 parts by mass of the base polymer (B). When the onium salt type quencher of component (D) is within the above range, the resolution is good and there is no significant decrease in sensitivity. This is therefore preferable. The onium salts represented by the general formula (2) or (3) may be used alone or in a combination of two or more types.

The chemically amplified resist composition of the present invention may contain a nitrogen-containing compound as a quencher (D). Examples of component (D), the nitrogen-containing compound, include primary, secondary, or tertiary amine compounds described in paragraphs [0146] to [0164] of JP2008-111103A, particularly amine compounds having a hydroxy group, an ether bond, an ester bond, a lactone ring, a cyano group, or a sulfonate ester bond. Further examples include compounds in which a primary or secondary amine is protected with a carbamate group, such as the compound described in JP3790649B2.

Furthermore, a sulfonium salt of a sulfonic acid having a nitrogen-containing substituent may be used as the nitrogen-containing compound. Such compounds function as quenchers in unexposed areas, and lose their quenching ability by neutralizing with the acid generated by themselves in exposed areas, functioning as so-called photodegradable bases. By using a photodegradable base, the contrast between exposed and unexposed areas can be further enhanced. Regarding the photodegradable base, reference can be made to, for example, JP2009-109595A or JP2012-046501A.

When the chemically amplified resist composition of the present invention contains a nitrogen-containing compound as a quencher (D), the content thereof is preferably 0.001 to 12 parts by mass, and more preferably 0.01 to 8 parts by mass, per 80 parts by mass of the base polymer (B). The nitrogen-containing compounds may be used alone or in a combination of two or more types.

### [Another Photoacid Generator (E)]

The chemically amplified resist composition of the present invention may contain, as component (E), a photoacid generator other than component (A) (hereinafter also referred to as “another photoacid generator”). The choice for another photoacid generator is not particularly limited as long as they are compounds that generate acid when irradiated with high-energy beams. Suitable choices for another photoacid generator include those represented by the following general formula (4) or (5).

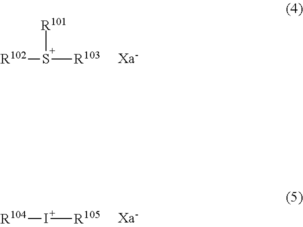

In the general formula (4), R¹⁰¹ to R¹⁰⁵ each independently represent a hydrocarbyl group having 1 to 20 carbon atoms and optionally containing a heteroatom. Also, any two of R¹⁰¹, R¹⁰² and R¹⁰³ may be bonded to each other to form a ring together with the sulfur atom to which they are bonded.

Examples of the cation of the sulfonium salt represented by the general formula (4) include those exemplified as specific examples of the sulfonium cation in the general formula (1B), those described in paragraphs [0102] to [0125] of JP2024-003744A, those described in paragraphs [0044] to [0049] of WO2024/128017A1, those described in paragraphs [0035] to [0046] of JP7491173B2, and those exemplified as specific examples of the sulfonium cation in the general formula (d3), but are not limited thereto. Specific examples of the cation of the iodonium salt represented by the general formula (5) include, but are not limited to, those described in paragraph [0181] of JP2024-000259A.

In the general formulae (4) and (5), Xa⁻ represents the anion of a strong acid. Examples of the anion of the strong acid include those represented by any of the general formulae (c1-1) to (c1-5).

Furthermore, as component (E), another photoacid generator, a compound represented by the following general formula (6) is also preferable.

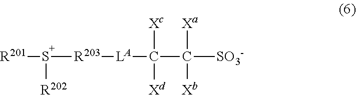

In the general formula (6), R²⁰¹ and R²⁰² each independently represent a hydrocarbyl group having 1 to 30 carbon atoms and optionally containing a heteroatom. R²⁰³ represents a hydrocarbylene group having 1 to 30 carbon atoms and optionally containing a heteroatom. Also, any two of R²⁰¹, R²⁰² and R²⁰³ may be bonded to each other to form a ring together with the sulfur atom to which they are bonded.

The hydrocarbyl group having 1 to 30 carbon atoms represented by R²⁰¹ and R²⁰² may be saturated or unsaturated, and may be linear, branched, or cyclic. Specific examples thereof include alkyl groups with 1 to 30 carbon atoms, such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl groups; cyclic saturated hydrocarbyl groups having 3 to 30 carbon atoms, such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}] decyl, and adamantyl groups; aryl groups having 6 to 30 carbon atoms, such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl groups; and groups obtained by combining these, etc. In addition, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

The hydrocarbylene group having 1 to 30 carbon atoms represented by R²⁰³ may be saturated or unsaturated, and may be linear, branched, or cyclic. Specific examples thereof include alkanediyl groups having 1 to 30 carbon atoms, such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl groups; cyclic saturated hydrocarbylene groups with 3 to 30 carbon atoms, such as cyclopentanediyl, cyclohexanediyl, norbornanediyl, and adamantanediyl groups; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene groups. In addition, some or all of the hydrogen atoms of the hydrocarbylene group may be substituted with a group containing a heteroatom such as an oxygen atom, a sulfur atom, a nitrogen atom, or a halogen atom, and some of the —CH₂— of the hydrocarbylene group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. The heteroatom is preferably an oxygen atom.

In the general formula (6), L^{A} represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include the same ones as those exemplified as the hydrocarbylene group represented by R²⁰³.

In the general formula (6), X^{a}, X^{b}, X^{c}, and X^{d} each independently represent a hydrogen atom, a fluorine atom, or a trifluoromethyl group. However, at least one of X^{a}, X^{b}, X^{c}, and X^{d} represents a fluorine atom or a trifluoromethyl group.

For the photoacid generator represented by the general formula (6), one represented by the following general formula (6′) is preferable.

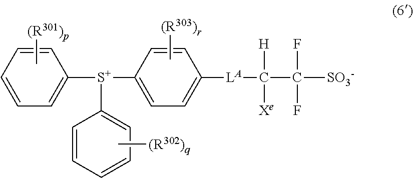

In the general formula (6′), L^{A} is as defined above. X^{e} represents a hydrogen atom or a trifluoromethyl group, and is preferably a trifluoromethyl group. R³⁰¹, R³⁰² and R³⁰³ each independently represent a hydrogen atom, or a hydrocarbyl group having 1 to 20 carbon atoms and optionally containing a heteroatom. The hydrocarbyl group may be saturated or unsaturated and may be linear, branched, or cyclic. Specific examples thereof include the same ones as those exemplified as the hydrocarbyl group represented by R^{fa1} in the general formula (c1-1-1). “p” and “q” each independently represent an integer from 0 to 5, and “r” represents an integer from 0 to 4.

Examples of the photoacid generator represented by the general formula (6) include the same ones as those exemplified as the photoacid generator represented by formula (2) in JP2017-026980A.

Among the other photoacid generators, those containing the anion represented by the general formula (c1-1-1) or (c1-4) are particularly preferable because they have low acid diffusion and excellent solubility in solvents. Moreover, ones represented by the general formula (6′) are particularly preferable because they have extremely low acid diffusion.

When the chemically amplified resist composition of the present invention contains another photoacid generator (E), the content thereof is preferably 0.1 to 40 parts by mass, and more preferably 0.5 to 20 parts by mass, per 80 parts by mass of the base polymer (B). When the amount of the component (E) photoacid generator added is within the above range, the resolution is good and there is no risk of problems with foreign matter occurring after development or during stripping of the resist film. This is therefore preferable. Another photoacid generator (E) may be used alone or in a combination of two or more types.

### [Surfactant (F)]

The chemically amplified resist composition of the present invention may further contain a surfactant as component (F). The surfactant (F) is preferably a surfactant that is insoluble or poorly soluble in water and soluble in an alkaline developer, or a surfactant that is insoluble or poorly soluble in water and an alkaline developer. For such surfactants, reference can be made to those described in JP2010-215608A and JP2011-016746A.

As surfactants that are insoluble or poorly soluble in water and alkaline developers, among the surfactants described in the above publications, preferable include FC-4430 (manufactured by 3M), Surflon (registered trademark), S-381 (manufactured by AGC Seimi Chemical Co., Ltd.), OLFINE (registered trademark) E1004 (manufactured by Nissin Chemical Co., Ltd.), KH-20 and KH-30 (manufactured by AGC Seimi Chemical Co., Ltd.), and oxetane ring-opening polymers represented by the following general formula (surf-1).

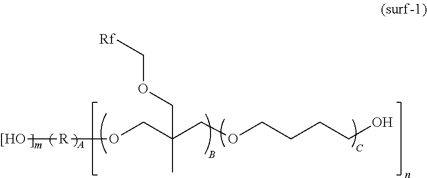

Here, R, Rf, A, B, C, “m”, and “n” apply only to the general formula (surf-1), regardless of the above description. R represents a divalent to tetravalent aliphatic group having 2 to 5 carbon atoms. For the aliphatic group, examples of divalent groups include an ethylene group, a 1,4-butylene group, a 1,2-propylene group, a 2,2-dimethyl-1,3-propylene group, and a 1,5-pentylene group, and examples of trivalent or tetravalent groups include the following.

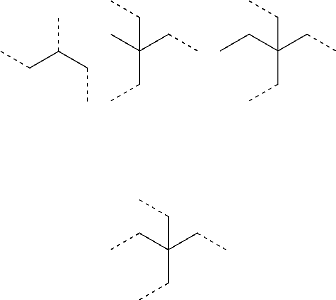

In the formulae, the dashed lines represent attachment points and are partial structures derived from glycerol, trimethylolethane, trimethylolpropane, and pentaerythritol, respectively.

Among these, 1,4-butylene group, 2,2-dimethyl-1,3-propylene group, etc. are preferable.

Rf represents a trifluoromethyl group or a pentafluoroethyl group, and is preferably a trifluoromethyl group. “m” represents an integer from 0 to 3, “n” represents an integer from 1 to 4, and the sum of “n” and “m” represents the valence of R and represents an integer from 2 to 4. A represents 1. B represents an integer from 2 to 25, and preferably an integer from 4 to 20. C represents an integer from 0 to 10, and is preferably 0 or 1. Furthermore, the order of the constituent units in the general formula (surf-1) is not specified, and they may be bonded in blocks or randomly. The production of part-fluorinated oxetane ring-opening polymer-based surfactants is described in detail in U.S. Pat. No. 5,650,483A.

Surfactants that are insoluble or poorly soluble in water but soluble in alkaline developers have the function of reducing water penetration and leaching by orienting themselves on the surface of the resist film when a resist protective film is not used in ArF immersion lithography. Therefore, they are useful for suppressing the elution of water-soluble components from the resist film and reducing damage to exposure equipment, and are also useful because they become soluble during alkaline aqueous solution development after exposure or post-exposure bake (PEB), making them less likely to become foreign matter that causes defects. Such surfactants are insoluble or poorly soluble in water and soluble in an alkaline developer, and are polymer-type surfactants that are also called hydrophobic resins; in particular, those that have high water repellency and improve water slippability are preferable.

Specific examples of such polymer-type surfactants include those containing at least one repeating unit selected from those represented by any of the following general formulae (7A) to (7E).

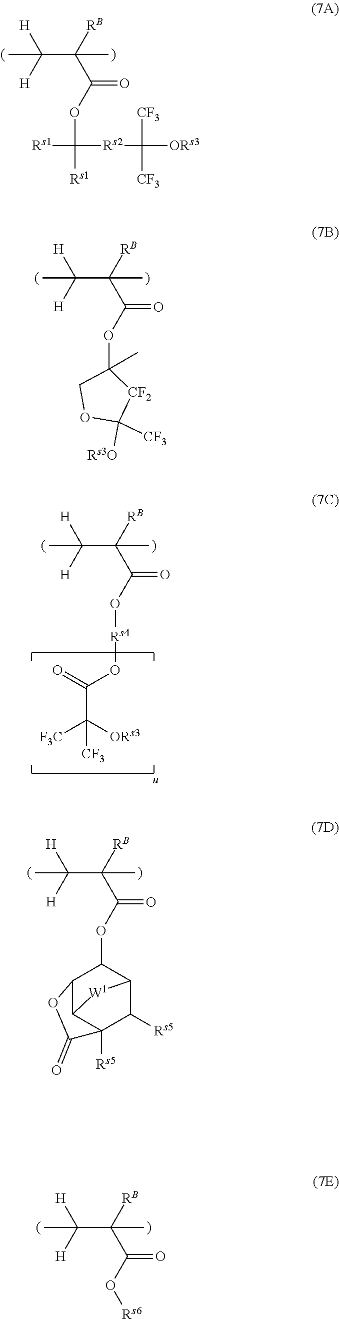

In the general formulae (7A) to (7E), R^{B} represents a hydrogen atom, a fluorine atom, a methyl group, or a trifluoromethyl group. W¹ represents —CH₂—, —CH₂CH₂—, —O—, or two separated —H. R^{s1} each independently represents a hydrogen atom, or a hydrocarbyl group having 1 to 10 carbon atoms. R^{s2} represents a single bond, or a linear or branched hydrocarbylene group having 1 to 5 carbon atoms. R^{s3} each independently represents a hydrogen atom, a hydrocarbyl group or a fluorinated hydrocarbyl group having 1 to 15 carbon atoms, or an acid-labile group. When R^{s3} is a hydrocarbyl group or a fluorinated hydrocarbyl group, an ether bond or a carbonyl group may be present between the carbon-carbon bonds. R^{s4} represents a (u+1)-valent hydrocarbon group or fluorinated hydrocarbon group having 1 to 20 carbon atoms. “u” represents an integer from 1 to 3. R^{s5} each independently represents a hydrogen atom, or a group represented by —C(═O)—O—R^{sa}. R^{sa} represents a fluorinated hydrocarbyl group having 1 to 20 carbon atoms. R^{s6} represents a hydrocarbyl group or a fluorinated hydrocarbyl group having 1 to 15 carbon atoms, and an ether bond or a carbonyl group may be present between the carbon-carbon bonds.

The hydrocarbyl group having 1 to 10 carbon atoms represented by R^{s1} is preferably a saturated hydrocarbyl group, and may be linear, branched, or cyclic. Specific examples thereof include alkyl groups having 1 to 10 carbon atoms, such as methyl, ethyl, n-propyl, isopropyl, n-butyl, isobutyl, sec-butyl, tert-butyl, n-pentyl, n-hexyl, n-heptyl, n-octyl, n-nonyl, and n-decyl groups; and cyclic saturated hydrocarbyl groups having 3 to 10 carbon atoms, such as cyclopropyl, cyclobutyl, cyclopentyl, cyclohexyl, adamantyl, and norbornyl groups. Of these, those having 1 to 6 carbon atoms are preferable.

The hydrocarbylene group represented by R^{s2} is preferably a saturated hydrocarbylene group, and may be linear, branched, or cyclic. Specific examples include a methylene group, an ethylene group, a propylene group, a butylene group, and a pentylene group.

The hydrocarbyl group represented by R^{s3} and R^{s6} may be saturated or unsaturated, and may be linear, branched, or cyclic. Specific examples thereof include saturated hydrocarbyl groups, or aliphatic unsaturated hydrocarbyl groups such as alkenyl groups and alkynyl groups, with saturated hydrocarbyl groups being preferable. Examples of the saturated hydrocarbyl group include, in addition to those exemplified as hydrocarbyl groups represented by R^{s1}, an undecyl group, a dodecyl group, a tridecyl group, a tetradecyl group, and a pentadecyl group. Examples of the fluorinated hydrocarbyl group represented by R^{s3} and R^{s6} include groups in which some or all of the hydrogen atoms bonded to carbon atoms of the aforementioned hydrocarbyl group have been substituted with fluorine atoms. As mentioned above, an ether bond or a carbonyl group may be present between these carbon-carbon bonds.

Specific examples of the acid-labile group represented by R^{s3} include groups represented by the general formulae (AL-1) to (AL-3), a trialkylsilyl group in which each alkyl group has 1 to 6 carbon atoms, and an oxo group-containing alkyl group having 4 to 20 carbon atoms.

The (u+1)-valent hydrocarbon group or fluorinated hydrocarbon group represented by R^{s4} may be linear, branched, or cyclic, and specific examples thereof include groups obtained by further desorbing u hydrogen atoms from the above-mentioned hydrocarbyl groups or fluorinated hydrocarbyl groups, etc.

The fluorinated hydrocarbyl group represented by R^{sa} is preferably saturated and may be linear, branched, or cyclic. Specific examples thereof include those in which some or all of the hydrogen atoms of the hydrocarbyl groups have been substituted with fluorine atoms, and specific examples thereof include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl groups.

Specific examples of the repeating units represented by any of the general formulae (7A) to (7E) include, but are not limited to, the following. Note that in the following formulae, R^{B} is as defined above.

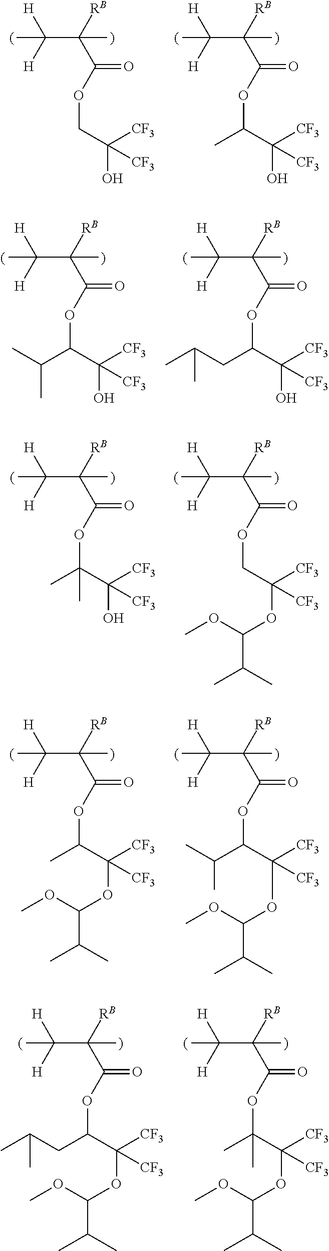

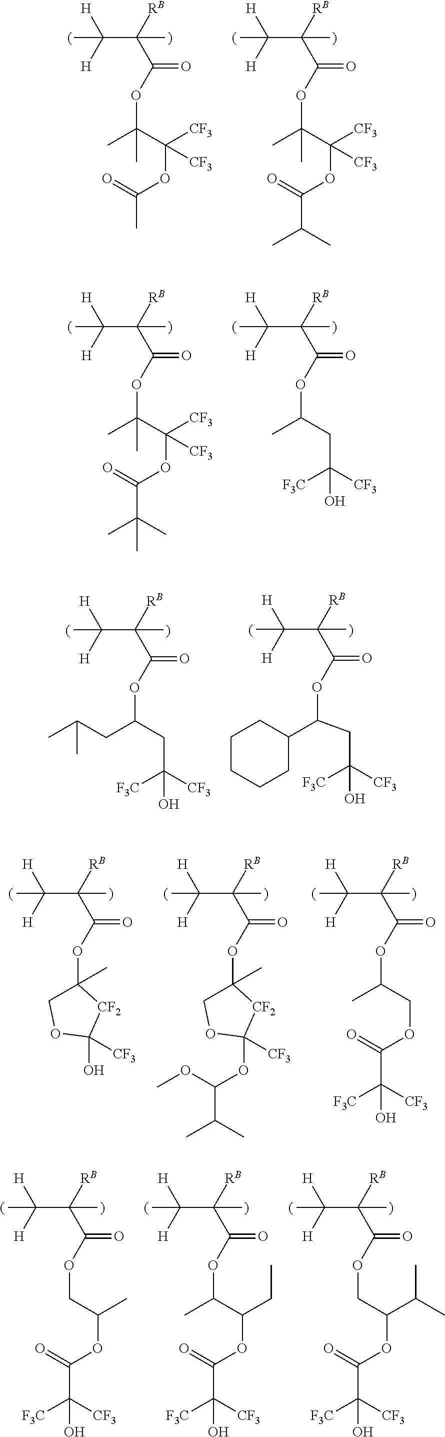

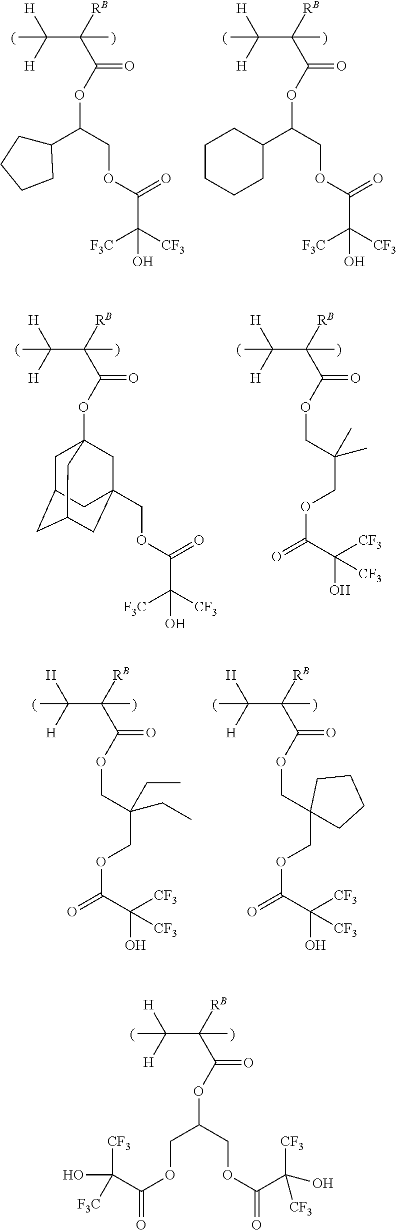

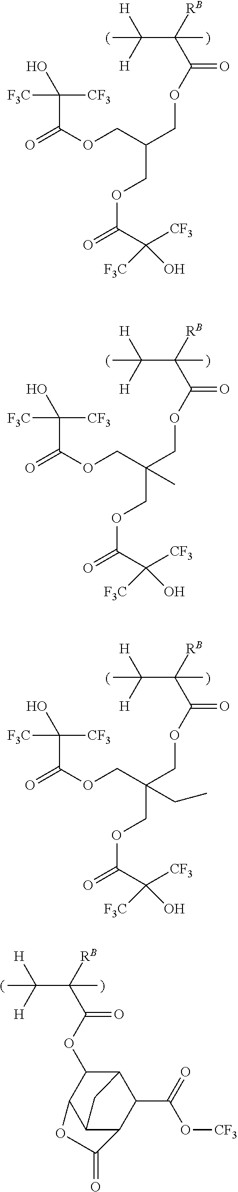

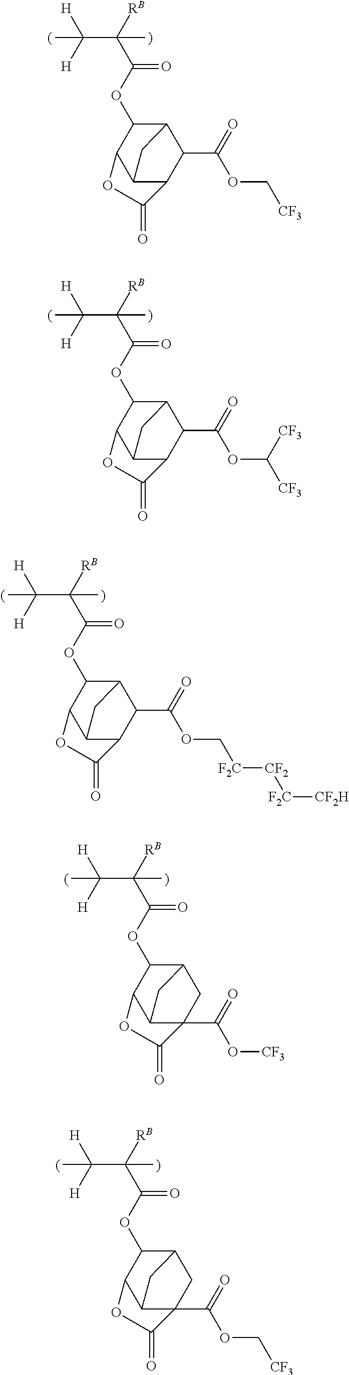

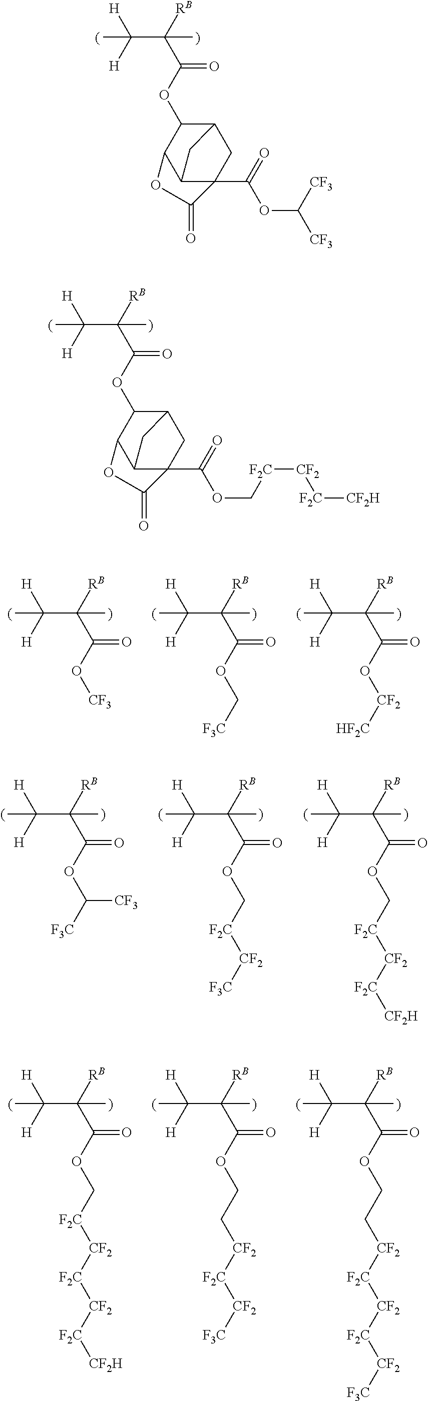

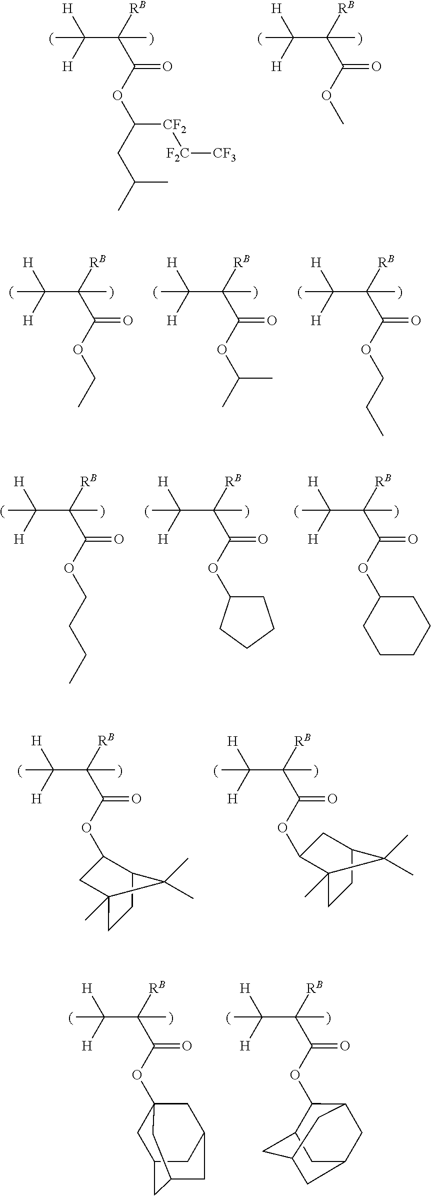

The polymer-type surfactant may further contain other repeating units other than the repeating units represented by the general formulae (7A) to (7E). Other repeating units include those derived from methacrylic acid and α-trifluoromethylacrylic acid derivatives, etc. In the polymer-type surfactant, the content of the repeating units represented by the general formulae (7A) to (7E) is preferably 20 mol % or more, more preferably 60 mol % or more, and even more preferably 100 mol %, of all repeating units.

The Mw of the polymer-type surfactant is preferably 1,000 to 500,000, and more preferably 3,000 to 100,000. Mw/Mn is preferably 1.0 to 2.0, and more preferably 1.0 to 1.6.

The polymer-type surfactant can be synthesized by heating a monomer containing an unsaturated bond that gives the repeating units represented by the general formulae (7A) to (7E) and, if necessary, other repeating units in an organic solvent in the presence of a radical initiator to polymerize the monomer. The organic solvents used during polymerization include toluene, benzene, THF, diethyl ether, dioxane, etc. Polymerization initiators include AIBN, 2,2′-azobis(2,4-dimethylvaleronitrile), dimethyl 2,2-azobis(2-methylpropionate), benzoyl peroxide, lauroyl peroxide, etc. The reaction temperature is preferably 50 to 100° C. The reaction time is preferably 4 to 24 hours. The acid-labile group may be used as is after being introduced into the monomer, or may be protected or partially protected after polymerization.

When synthesizing the polymer-type surfactant, a known chain transfer agent such as dodecyl mercaptan or 2-mercaptoethanol may be used to adjust the molecular weight. In this case, the amount of these chain transfer agents added is preferably 0.01 to 10 mol % based on the total number of moles of the monomers to be polymerized.

When the chemically amplified resist composition of the present invention contains the surfactant (F), the content thereof is preferably 0.1 to 50 parts by mass, and more preferably 0.5 to 10 parts by mass, per 80 parts by mass of the base polymer (B). When the content of the surfactant (F) is 0.1 parts by mass or more, the receding contact angle between the resist film surface and water is sufficiently improved, and when it is 50 parts by mass or less, the dissolution rate of the resist film surface in the developer is low, and the height of the formed fine pattern is sufficiently maintained. The surfactants (F) may be used alone or in a combination of two or more types.

### [Other Components (G)]

The chemically amplified resist composition of the present invention may contain, as other components (G), a compound that decomposes when exposed to acid to generate acid (acid amplifying compound), an organic acid derivative, a fluoro-substituted alcohol, a compound having an Mw of 3,000 or less whose solubility in a developer changes due to the action of acid (dissolution inhibitor), etc. For the acid amplifying compound, reference can be made to the compounds described in JP2009-269953A or JP2010-215608A. When the acid amplifying compound is included, the content thereof is preferably 0 to 5 parts by mass, and more preferably 0 to 3 parts by mass, per 80 parts by mass of the base polymer (B). When the content falls within this range, it is possible to control acid diffusion, and degradation of resolution and pattern shape is unlikely to occur. For the organic acid derivative, fluoro-substituted alcohol, and dissolution inhibitor, reference can be made to the compounds described in JP2009-269953A or JP2010-215608A.

### [Patterning Process]

The present invention also provides a patterning process, comprising the steps of: forming a resist film on a substrate using the above-described chemically amplified resist composition; exposing the resist film with a high-energy beam; and developing the exposed resist film using a developer.

For the substrate, for example, a substrate for manufacturing integrated circuits (Si, SiO₂, SiN, SiON, TiN, WSi, BPSG, SOG, organic anti-reflective coating, etc.) or a substrate for mask circuit manufacturing (Cr, CrO, CrON, MoSi₂, SiO₂, etc.) can be used.

The resist film can be formed, for example, by applying the chemically amplified resist composition onto a substrate by a method such as spin coating so that the film thickness becomes preferably 0.05 to 2 μm, and then pre-baking the applied composition on a hot plate preferably at 60 to 150° C. for 1 to 10 minutes, and more preferably at 80 to 140° C. for 1 to 5 minutes.

As the high-energy beam used for exposing the resist film, it is preferable to use KrF excimer laser light, ArF excimer laser light, electron beam (EB), or extreme ultraviolet (EUV) having a wavelength of 3 to 15 nm. When using KrF excimer laser light, ArF excimer laser light, or EUV for exposure, a mask can be used to form the desired pattern, and irradiation can be performed so that the exposure amount is preferably 1 to 200 mJ/cm², and more preferably 10 to 100 mJ/cm². When using EB, irradiation is performed using a mask for forming the desired pattern or directly so that the exposure amount is preferably 1 to 300 μC/cm², and more preferably 10 to 200 μC/cm².

In addition to the usual exposure methods, an immersion method can also be used for exposure, in which a liquid with a refractive index of 1.0 or more is placed between the resist film and the projection lens. In that case, it is possible to use a water-insoluble protective film.

The water-insoluble protective films are used to prevent substances from eluting from the resist film and to increase the water slippability of the film surface, and are broadly divided into two types. One is an organic solvent stripping type that needs to be stripped before alkaline aqueous solution development using an organic solvent that does not dissolve the resist film, and the other is an alkaline aqueous solution soluble type that is soluble in alkaline developer and removes the protective film along with removing the soluble parts of the resist film. For the latter, a particularly preferable choice is a material based on a polymer having a 1,1,1,3,3,3-hexafluoro-2-propanol residue, which is insoluble in water but soluble in an alkaline developer, and dissolved in an alcohol solvent having 4 or more carbon atoms, an ether solvent having 8 to 12 carbon atoms, or a mixed solvent of these. For the above-mentioned water-insoluble, alkaline developer-soluble surfactant, materials can also be used that have been dissolved in an alcohol solvent having 4 or more carbon atoms, an ether solvent having 8 to 12 carbon atoms, or a mixed solvent of these.

After exposure, PEB may be performed. PEB can be performed, for example, by heating on a hot plate, preferably at 60 to 150° C. for 1 to 5 minutes, and more preferably at 80 to 140° C. for 1 to 3 minutes.

The development is carried out, for example, using a developer that is an alkaline aqueous solution such as tetramethylammonium hydroxide (TMAH) of preferably 0.1 to 5% by mass, and more preferably 2 to 3% by mass, and by a conventional method such as a dip method, a puddle method, or a spray method for preferably 0.1 to 3 minutes, and more preferably 0.5 to 2 minutes, so that the exposed area dissolves and the desired pattern is formed on the substrate.

After the resist film is formed, a pure water rinse may be performed to extract the acid generator, etc. from the film surface or to wash away particles, and a rinse may be performed to remove water remaining on the film after exposure.

Furthermore, patterns may be formed by a double patterning method. Examples of the double patterning method include the trench method, in which a first exposure and etching process processes the base of a 1:3 trench pattern, then the position is shifted and a second exposure forms a 1:3 trench pattern to form a 1:1 pattern, and the line method, in which a first base of a 1:3 isolated left pattern is processed by a first exposure and etching process, then a position shift and a second exposure forms a 1:3 isolated left pattern under the first base to form a second base, and then the second base is processed to form a 1:1 pattern with half the pitch.

In the patterning process of the present invention, a negative tone development method may be used, in which an organic solvent is used as a developer instead of the alkaline aqueous solution to dissolve the unexposed areas.

For the negative tone development, the following developers can be used: 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, etc. These organic solvents may be used alone or in a combination of two or more types.

### EXAMPLE

The present invention will be described in detail below with reference to Synthesis Examples, Examples, and Comparative Examples, but the present invention is not limited to the following Examples. The equipment used is as follows:
MALDI TOF-MS: S3000 manufactured by JEOL Ltd.

### [1] Synthesis of Onium Salts

### [Example 1-1] Synthesis of Onium Salt PAG-1

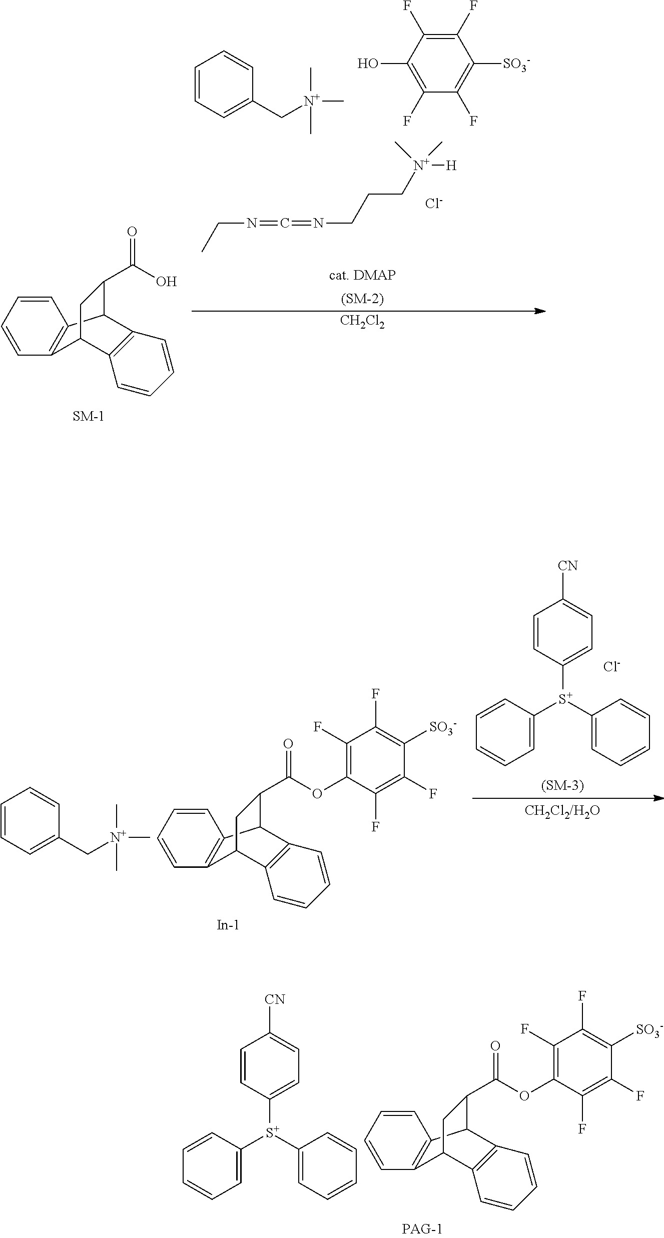

### (1) Synthesis of Intermediate In-1

Under a nitrogen atmosphere, raw materials SM-1 (6.3 g), SM-2 (10.4 g), DMAP (4-dimethylaminopyridine, 0.3 g), and methylene chloride (50 g) were added to a reaction vessel and cooled in an ice bath. While maintaining the temperature inside the reaction vessel at 20° C. or less, 1-ethyl-3-(3-dimethylaminopropyl)carbodiimide hydrochloride (5.8 g) was added as a powder. After the addition, the temperature was raised to room temperature and aged for 12 hours. After aging, water was added to stop the reaction, the reaction was then subjected to the usual aqueous work-up, then after the solvent was distilled off, diisopropyl ether was added to wash the residue, yielding 20.4 g of intermediate In-1 as an oily substance (yield: 92%).

### (2) Synthesis of PAG-1

Under a nitrogen atmosphere, intermediate In-1 (14.4 g), an aqueous solution of raw material SM-3 (30.5 g, equivalent to 25.1 mol), and methylene chloride (50 g) were added and stirred for 15 minutes, and then the organic layer was separated and washed with water, and then concentrated under reduced pressure. Methyl isobutyl ketone (50 g) was added to the concentrated liquid and azeotropic dehydration was carried out, and diisopropyl ether was further added to crystallize it, obtaining 16.7 g of the target product, PAG-1, as white crystals (yield: 95%).
MALDI TOF-MS: POSITIVE M⁺ 288 (C₁₉H₁₄NS⁺ equivalent)
NEGATIVE M⁻ 477 (C₂₃H₁₃F₄O₅S⁻ equivalent)

### [Examples 1-2 to 1-9] Synthesis of Onium Salts PAG-2 to PAG-9

Using the corresponding raw materials and known organic synthesis reactions, onium salts PAG-2 to PAG-9 represented by the following formulae were synthesized.

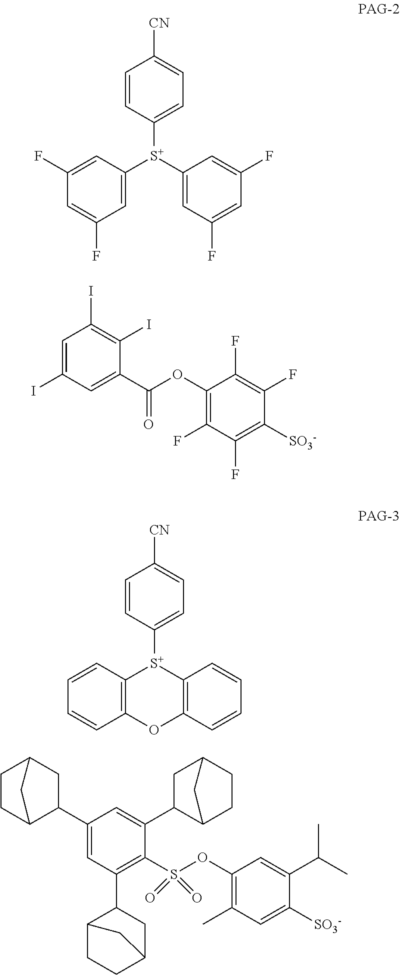

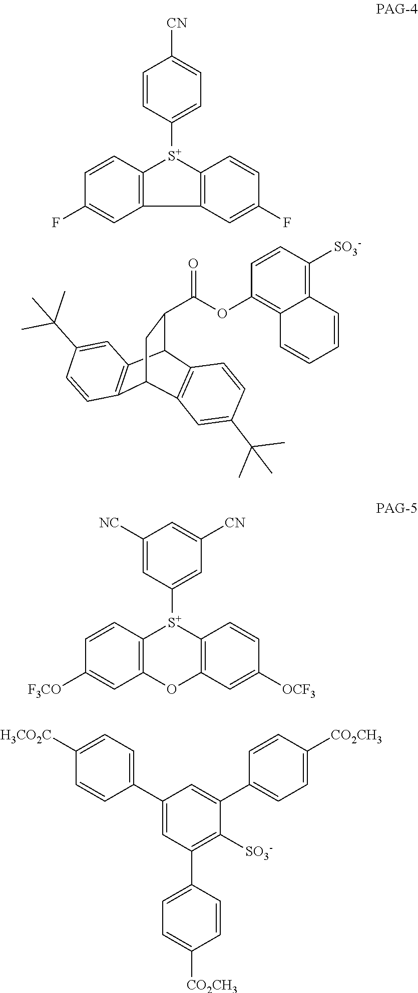

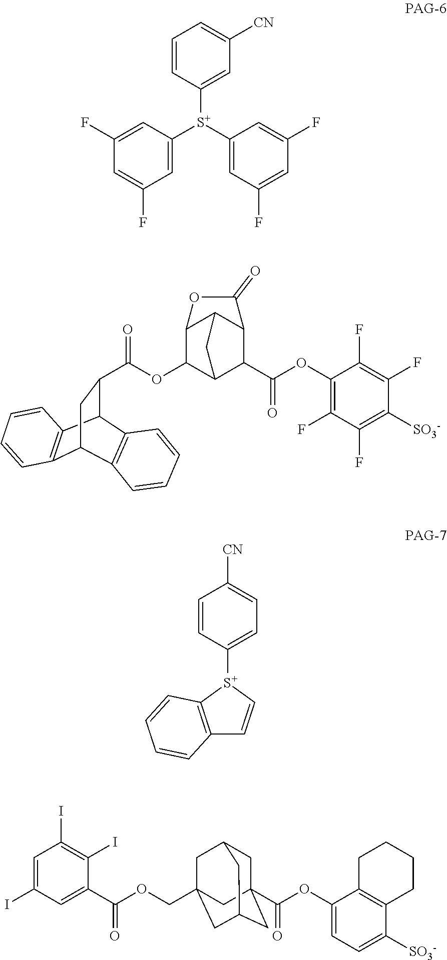

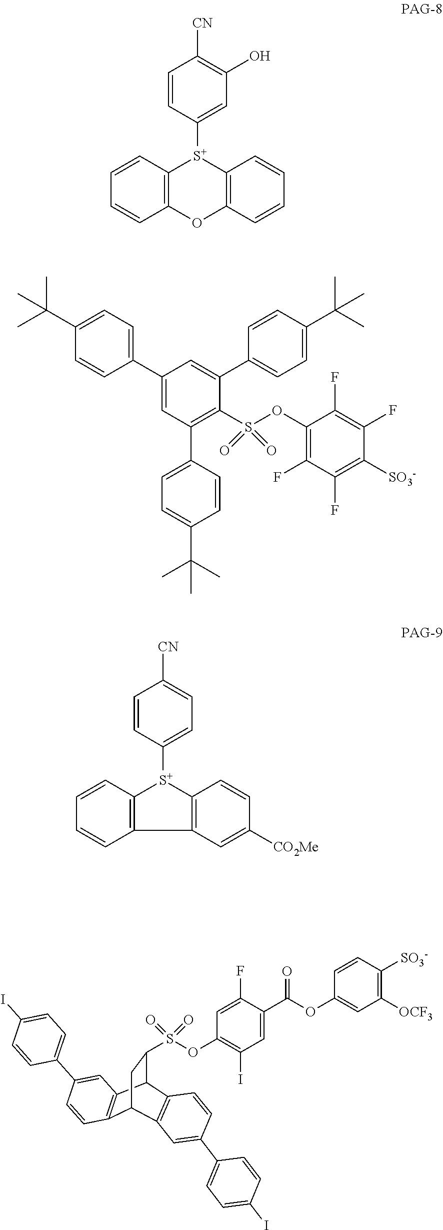

### [2] Synthesis of Base Polymer

### [Synthesis Examples 1 to 5] Synthesis of Base Polymers (P-1 to P-5)

Each monomer was combined and copolymerized in MEK, which is a solvent, and the reaction solution was poured into hexane; the precipitated solid was washed with hexane, isolated, and dried to obtain the base polymers (P-1 to P-5) with the compositions shown below. The compositions of the resulting base polymers were confirmed by 1H-NMR, and their Mw and Mw/Mn were confirmed by GPC (solvent: THF, standard: polystyrene).

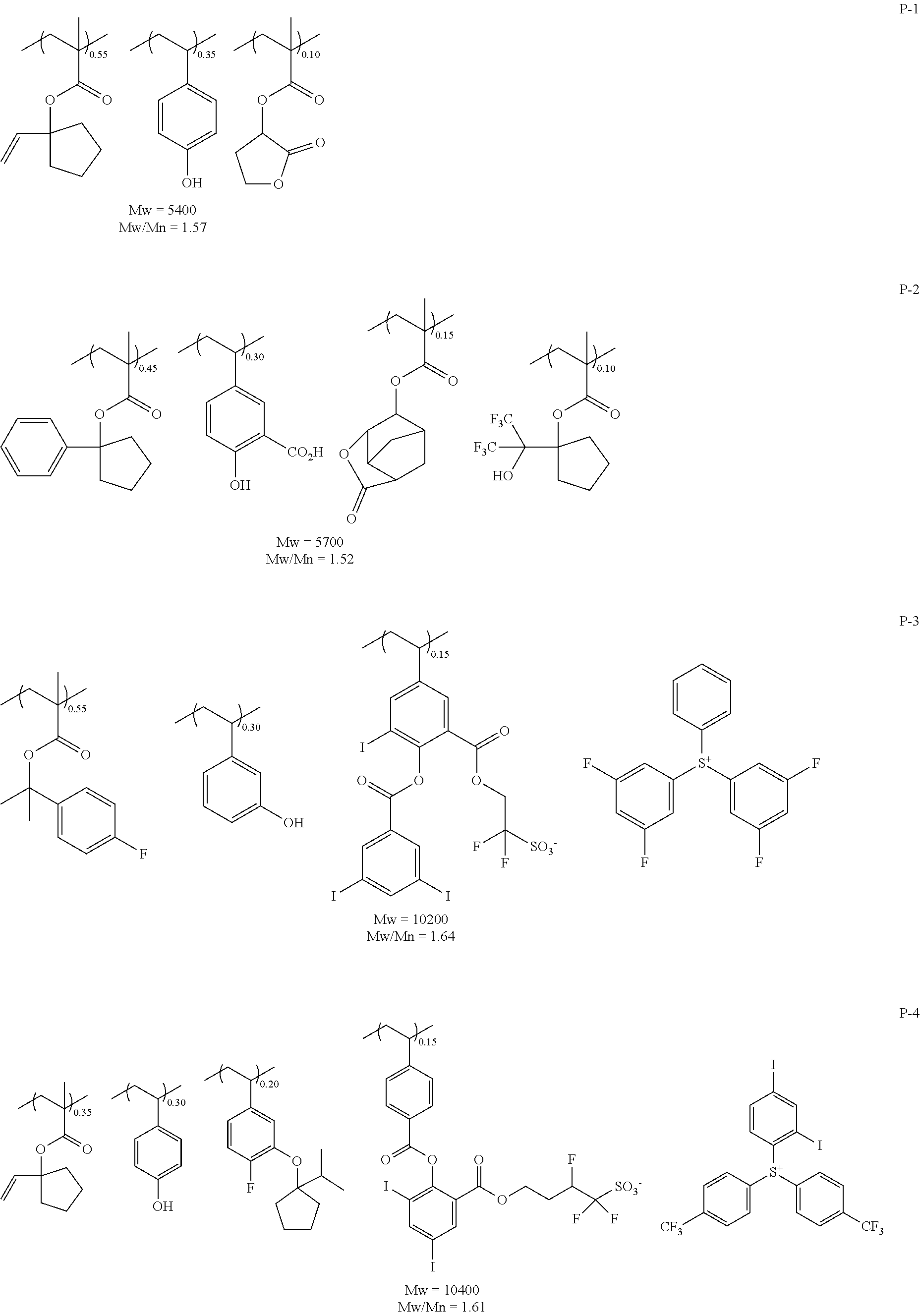

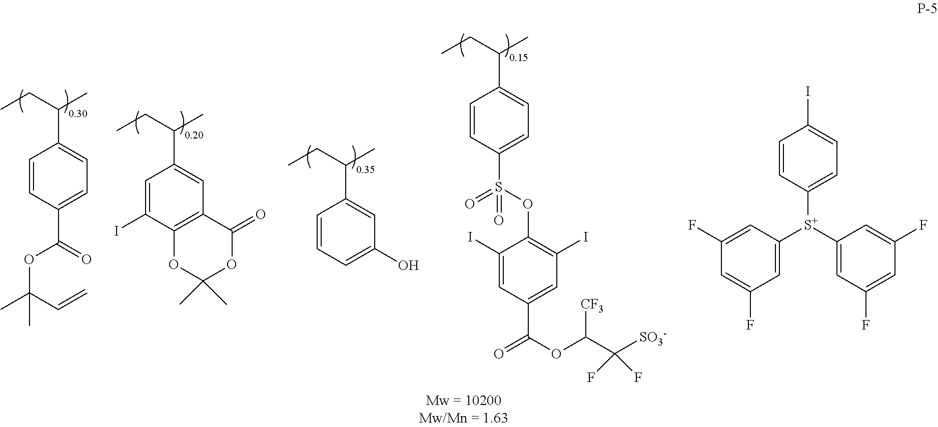

### [3] Preparation of Chemically Amplified Resist

### Composition

### Examples 2-1 to 2-30, Comparative Examples 1-1 to 1-20

Photoacid generators (PAG-1 to PAG-9) consisting of onium salts of the present invention, comparative photoacid generators (PAG-A to PAG-E), another photoacid generator (PAG-X), base polymers (P-1 to P-5), and quenchers (Q-1 to Q-4) were dissolved in a solvent containing 0.01% by mass of surfactant A (OMNOVA) with the compositions shown in Tables 1 and 2 below to prepare solutions, which were then filtered through a 0.2 μm Teflon (registered trademark)-type filter to prepare chemically amplified resist compositions (R-1 to R-30 and CR-1 to CR-20).

TABLE 1

| | | Base | | Another | | Solvent | Solvent | Solvent |
|---|---|---|---|---|---|---|---|---|
| | | polymer | Photoacid | photoacid | | 1 | 2 | 3 |
| | | (parts | generator | generator | Quencher | (parts | (parts | (parts |
| | Resist | by | (parts by | (parts by | (parts by | by | by | by |
| | composition | mass) | mass) | mass) | mass) | mass) | mass) | mass) |
| Example | R-1 | P-1 (80) | PAG-1 (28) | — | Q-1 (8.0) | PGMEA | EL | DAA |
| 2-1 | | | | | | (2250) | (2800) | (550) |
| Example | R-2 | P-1 (80) | PAG-2 (28) | — | Q-1 (7.8) | PGMEA | EL | DAA |
| 2-2 | | | | | | (2250) | (2800) | (550) |
| Example | R-3 | P-1 (80) | PAG-3 (29) | — | Q-1 (7.4) | PGMEA | EL | DAA |
| 2-3 | | | | | | (2250) | (2800) | (550) |
| Example | R-4 | P-1 (80) | PAG-4 (27) | — | Q-1 (8.0) | PGMEA | EL | DAA |
| 2-4 | | | | | | (2250) | (2800) | (550) |
| Example | R-5 | P-1 (80) | PAG-5 (15) | PAG-X (10) | Q-1 (8.0) | PGMEA | EL | DAA |
| 2-5 | | | | | | (2250) | (2800) | (550) |
| Example | R-6 | P-1 (80) | PAG-6 (28) | — | Q-1 (8.0) | PGMEA | EL | DAA |
| 2-6 | | | | | | (2250) | (2800) | (550) |
| Example | R-7 | P-1 (80) | PAG-7 (28) | — | Q-1 (7.8) | PGMEA | EL | DAA |
| 2-7 | | | | | | (2250) | (2800) | (550) |
| Example | R-8 | P-1 (80) | PAG-8 (29) | — | Q-1 (7.4) | PGMEA | EL | DAA |
| 2-8 | | | | | | (2250) | (2800) | (550) |
| Example | R-9 | P-1 (80) | PAG-9 (15) | PAG-X (8) | Q-1 (8.0) | PGMEA | EL | DAA |
| 2-9 | | | | | | (2250) | (2800) | (550) |
| Example | R-10 | P-2 (80) | PAG-1 (29) | — | Q-1 (8.0) | PGMEA | EL | DAA |
| 2-10 | | | | | | (2250) | (2800) | (550) |
| Example | R-11 | P-2 (80) | PAG-2 (30) | — | Q-3 (7.8) | PGMEA | EL | DAA |
| 2-11 | | | | | | (2250) | (2800) | (550) |
| Example | R-12 | P-2 (80) | PAG-4 (28) | — | Q-1 (8.0) | PGMEA | EL | DAA |
| 2-12 | | | | | | (2250) | (2800) | (550) |
| Example | R-13 | P-2 (80) | PAG-5 (18) | PAG-X (7) | Q-1 (8.0) | PGMEA | EL | DAA |
| 2-13 | | | | | | (2250) | (2800) | (550) |
| Example | R-14 | P-2 (80) | PAG-7 (28) | — | Q-2 (4.0) | PGMEA | EL | DAA |
| 2-14 | | | | | Q-4 (4.0) | (2250) | (2800) | (550) |
| Example | R-15 | P-3 (80) | PAG-1 (10) | — | Q-1 (8.0) | PGMEA | EL | DAA |
| 2-15 | | | | | | (2250) | (2800) | (550) |
| Example | R-16 | P-3 (80) | PAG-2 (9) | — | Q-3 (7.6) | PGMEA | EL | DAA |
| 2-16 | | | | | | (2250) | (2800) | (550) |
| Example | R-17 | P-3 (80) | PAG-5 (8) | PAG-X (4) | Q-1 (7.8) | PGMEA | EL | DAA |
| 2-17 | | | | | | (2250) | (2800) | (550) |
| Example | R-18 | P-3 (80) | PAG-7 (9) | — | Q-3(8.0) | PGMEA | EL | DAA |
| 2-18 | | | | | | (2250) | (2800) | (550) |
| Example | R-19 | P-3 (80) | PAG-9 (8) | PAG-X (4) | Q-2 (8.0) | PGMEA | EL | DAA |
| 2-19 | | | | | | (2250) | (2800) | (550) |
| Example | R-20 | P-4 (80) | PAG-1 (10) | — | Q-1 (8.0) | PGMEA | EL | DAA |
| 2-20 | | | | | | (2250) | (2800) | (550) |
| Example | R-21 | P-4 (80) | PAG-3 (10) | — | Q-1 (7.6) | PGMEA | EL | DAA |
| 2-21 | | | | | | (2250) | (2800) | (550) |
| Example | R-22 | P-4 (80) | PAG-4 (8) | PAG-X (4) | Q-3 (8.0) | PGMEA | EL | DAA |
| 2-22 | | | | | | (2250) | (2800) | (550) |
| Example | R-23 | P-4 (80) | PAG-6 (10) | — | Q-2(8.0) | PGMEA | EL | DAA |
| 2-23 | | | | | | (2250) | (2800) | (550) |
| Example | R-24 | P-4 (80) | PAG-8 (8) | — | Q-3 (4.0) | PGMEA | EL | DAA |
| 2-24 | | | | | Q-4 (4.0) | (2250) | (2800) | (550) |
| Example | R-25 | P-5 (80) | PAG-1 (10) | — | Q-1 (8.0) | PGMEA | EL | DAA |
| 2-25 | | | | | | (2250) | (2800) | (550) |
| Example | R-26 | P-5 (80) | PAG-2 (8) | — | Q-2 (8.0) | PGMEA | EL | DAA |
| 2-26 | | | | | | (2250) | (2800) | (550) |
| Example | R-27 | P-5 (80) | PAG-4 (8) | — | Q-3 (7.8) | PGMEA | EL | DAA |
| 2-27 | | | | | | (2250) | (2800) | (550) |
| Example | R-28 | P-5 (80) | PAG-6 (10) | — | Q-1 (4.0) | PGMEA | EL | DAA |
| 2-28 | | | | | Q-4 (4.0) | (2250) | (2800) | (550) |
| Example | R-29 | P-5 (80) | PAG-7 (10) | — | Q-3 (8.0) | PGMEA | EL | DAA |
| 2-29 | | | | | | (2250) | (2800) | (550) |
| Example | R-30 | P-5 (80) | PAG-9 (8) | PAG-X (4) | Q-2 (7.6) | PGMEA | EL | DAA |
| 2-30 | | | | | | (2250) | (2800) | (550) |

TABLE 2

| | | Base | | Another | | Solvent | Solvent | Solvent |
|---|---|---|---|---|---|---|---|---|
| | | polymer | Photoacid | photoacid | | 1 | 2 | 3 |
| | | (parts | generator | generator | Quencher | (parts | (parts | (parts |
| | Resist | by | (parts by | (parts by | (parts | by | by | by |
| | composition | mass ) | mass) | mass) | by mass) | mass) | mass) | mass) |
| Comparative | CR-1 | P-1 (80) | PAG-A (28) | — | Q-1 (8.0) | PGMEA | EL | DAA |
| Example 1-1 | | | | | | (2250) | (2800) | (550) |
| Comparative | CR-2 | P-1 (80) | PAG-B (28) | — | Q-1 (7.8) | PGMEA | EL | DAA |
| Example 1-2 | | | | | | (2250) | (2800) | (550) |
| Comparative | CR-3 | P-1 (80) | PAG-C (29) | — | Q-1 (7.8) | PGMEA | EL | DAA |
| Example 1-3 | | | | | | (2250) | (2800) | (550) |
| Comparative | CR-4 | P-1 (80) | PAG-D (27) | — | Q-1 (8.2) | PGMEA | EL | DAA |
| Example 1-4 | | | | | | (2250) | (2800) | (550) |
| Comparative | CR-5 | P-1 (80) | PAG-E (15) | PAG-X (10) | Q-1 (8.2) | PGMEA | EL | DAA |
| Example 1-5 | | | | | | (2250) | (2800) | (550) |
| Comparative | CR-6 | P-2 (80) | PAG-A (27) | — | Q-2 (8.0) | PGMEA | EL | DAA |
| Example 1-6 | | | | | | (2250) | (2800) | (550) |
| Comparative | CR-7 | P-2 (80) | PAG-B (25) | — | Q-1 (8.0) | PGMEA | EL | DAA |
| Example 1-7 | | | | | | (2250) | (2800) | (550) |
| Comparative | CR-8 | P-2 (80) | PAG-E (18) | PAG-X (7) | Q-1 (8.0) | PGMEA | EL | DAA |
| Example 1-8 | | | | | | (2250) | (2800) | (550) |
| Comparative | CR-9 | P-3 (80) | PAG-A (10) | — | Q-1 (7.8) | PGMEA | EL | DAA |
| Example 1-9 | | | | | | (2250) | (2800) | (550) |
| Comparative | CR-10 | P-3 (80) | PAG-B (9) | — | Q-3 (8.0) | PGMEA | EL | DAA |
| Example 1-10 | | | | | | (2250) | (2800) | (550) |
| Comparative | CR-11 | P-3 (80) | PAG-C (10) | — | Q-1 (4.0) | PGMEA | EL | DAA |
| Example 1-11 | | | | | Q-4 (4.0) | (2250) | (2800) | (550) |
| Comparative | CR-12 | P-3 (80) | PAG-D (8) | — | Q-2 (7.6) | PGMEA | EL | DAA |
| Example 1-12 | | | | | | (2250) | (2800) | (550) |
| Comparative | CR-13 | P-4 (80) | PAG-A (10) | — | Q-1 (7.8) | PGMEA | EL | DAA |
| Example 1-13 | | | | | | (2250) | (2800) | (550) |
| Comparative | CR-14 | P-4 (80) | PAG-B (10) | — | Q-2 (8.0) | PGMEA | EL | DAA |
| Example 1-14 | | | | | | (2250) | (2800) | (550) |
| Comparative | CR-15 | P-4 (80) | PAG-C (10) | — | Q-3 (4.0) | PGMEA | EL | DAA |
| Example 1-15 | | | | | Q-4 (4.0) | (2250) | (2800) | (550) |
| Comparative | CR-16 | P-4 (80) | PAG-E (8) | PAG-X (4) | Q-1 (8.0) | PGMEA | EL | DAA |
| Example 1-16 | | | | | | (2250) | (2800) | (550) |
| Comparative | CR-17 | P-5 (80) | PAG-A (10) | — | Q-1 (8.0) | PGMEA | EL | DAA |
| Example 1-17 | | | | | | (2250) | (2800) | (550) |
| Comparative | CR-18 | P-5 (80) | PAG-B (10) | — | Q-3 (7.6) | PGMEA | EL | DAA |
| Example 1-18 | | | | | | (2250) | (2800) | (550) |
| Comparative | CR-19 | P-5 (80) | PAG-C (8) | — | Q-1 (4.0) | PGMEA | EL | DAA |
| Example 1-19 | | | | | Q-4 (4.0) | (2250) | (2800) | (550) |
| Comparative | CR-20 | P-5 (80) | PAG-E (8) | PAG-X (4) | Q-2 (8.0) | PGMEA | EL | DAA |
| Example 1-20 | | | | | | (2250) | (2800) | (550) |

In Tables 1 and 2, the solvents, another photoacid generator PAG-X, comparative photoacid generators PAG-A to PAG-E, quenchers Q-1 to Q-4, and surfactant A are as follows.

### Solvent: PGMEA (Propylene Glycol Monomethyl Ether Acetate)

EL (Ethyl lactate)
DAA (Diacetone alcohol)
   Another photoacid generator: PAG-X

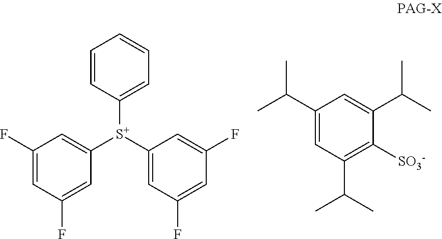

### Comparative Photoacid Generators: PAG-A to PAG-E

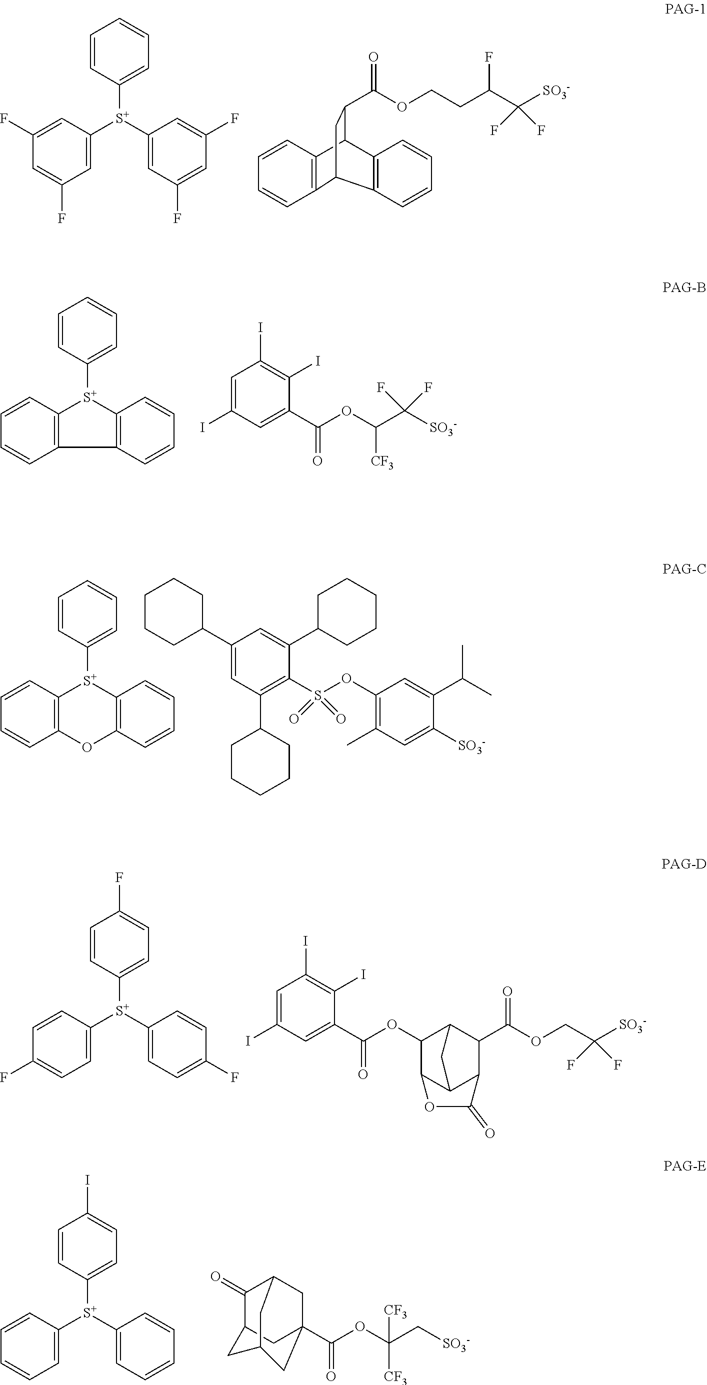

### Quenchers: Q-1 to Q-4

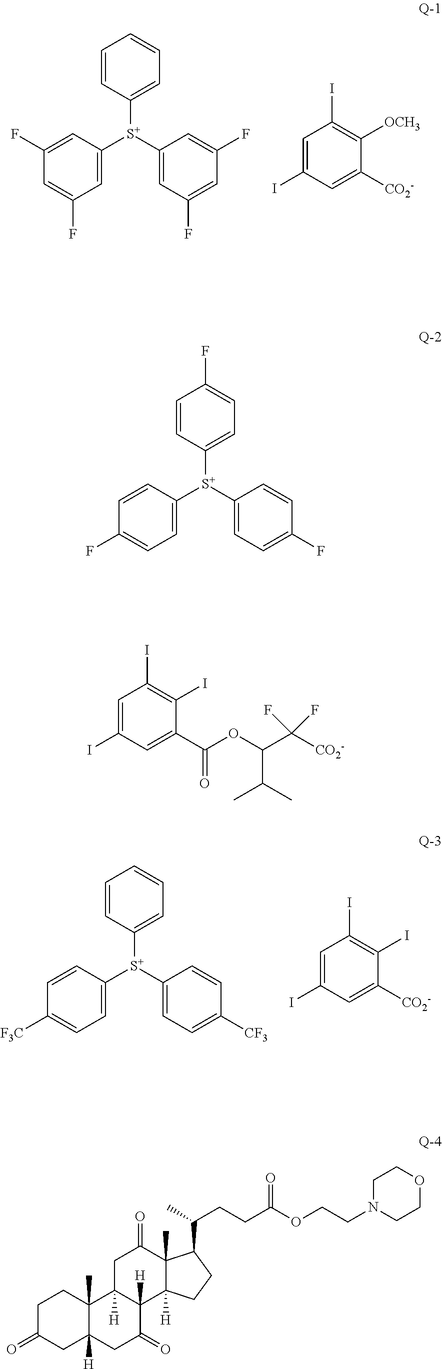

Surfactant A: Copolymer of 3-methyl-3-(2,2,2-trifluoroethoxymethyl)oxetane, tetrahydrofuran, and 2,2-dimethyl-1,3-propanediol (OMNOVA)

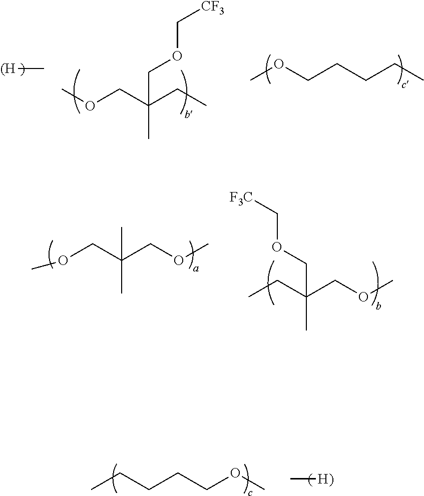

$$a : ( b + {b}^{′} ) : ( c + {c}^{′} ) = 1 : 4 to 7 : 0.01 to 1 ( molar ratio )$$

$$Mw = 1 , 500$$

### [4] EUV Lithography Evaluation (1)

### Examples 3-1 to 3-30, Comparative Examples 2-1 to 2-20

Each chemically amplified resist composition (R-1 to R-30, CR-1 to CR-20) shown in Tables 1 and 2 was spin-coated onto an Si substrate on which a 20 nm-thick silicon-containing spin-on hard mask SHB-A940 (silicon content: 43% by mass) manufactured by Shin-Etsu Chemical Co., Ltd. had been formed, and the substrate was pre-baked at 100° C. for 60 seconds using a hot plate to produce a resist film with a thickness of 50 nm. The resist film was exposed to an LS pattern with an on-wafer dimension of 18 nm and a pitch of 36 nm using an EUV scanner NXE:3400 (NA 0.33, σ 0.9/0.6, dipole illumination) manufactured by ASML, while changing the exposure amount and focus (exposure amount pitch: 1 mJ/cm², focus pitch: 0.020 μm); and after the exposure, PEB was performed for 60 seconds at the temperatures shown in Tables 3 and 4. After that, paddle development was performed for 30 seconds using a 2.38% by mass TMAH aqueous solution, followed by rinsing with a surfactant-containing rinse material and spin drying to obtain a positive pattern.

The obtained LS pattern was observed with a CD-SEM (CG6300) manufactured by Hitachi High-Tech Corporation, and the sensitivity, EL, LWR, depth of focus (DOF), and tilt limit were evaluated according to the following methods. In addition, the development defects of the obtained LS patterns were evaluated. The results are shown in Tables 3 and 4.

### [Sensitivity Evaluation]

The optimal exposure amount Eop (mJ/cm²) for obtaining an LS pattern with a line width of 18 nm and a pitch of 36 nm was calculated and used as the sensitivity. The smaller this value, the higher the sensitivity.

### [EL Evaluation]

The EL (unit: %) was calculated from the exposure amount formed within the range of ±10% (16.2 to 19.8 nm) of the 18 nm space width in the LS pattern using the following formula. The higher this value, the better the performance.

$$EL ( \% ) = ( \vert {E}_{1} - {E}_{2} \vert / Eop ) \times 100$$

E₁: Optimal exposure amount for LS pattern with line width of 16.2 nm and pitch of 36 nm
E₂: Optimal exposure amount for LS pattern with line width of 19.8 nm and pitch of 36 nm
Eop: Optimal exposure amount for LS pattern with line width of 18 nm and pitch of 36 nm

### [LWR Evaluation]

The dimensions of the LS pattern obtained by irradiating with Eop were measured at 10 points along the longitudinal direction of the line, the standard deviation (σ) was calculated, its value was tripled (3σ), and the result was used as the LWR. The smaller this value, the less roughness the pattern that can be obtained will have and the more uniform its line width will be.

### [DOF Evaluation]

To evaluate the depth of focus, the focus range formed within ±10% (16.2 to 19.8 nm) of the 18 nm dimension in the LS pattern was obtained. The larger this value, the wider the depth of focus.

### [Line Pattern Tilt Limit Evaluation]

The line dimensions of the LS pattern at each exposure amount at the optimal focus were measured at 10 points in the longitudinal direction. The thinnest line dimension that could be obtained without collapse was taken as the tilt limit dimension. The smaller this value, the better the tilt limit.

### [Development Defect Evaluation]

The LS pattern with a line width of 18 nm and a pitch of 36 nm formed with the above-mentioned optimal exposure amount was inspected using a defect inspection device KLA2360 (product name) manufactured by KLA Corporation by setting the pixel size of the defect inspection device to 0.16 μm and the threshold value to 20, the number of defects (items/cm²) extracted from the difference caused by overlaying the comparison image on a pixel-by-pixel basis was counted, and the number of defects per unit area (items/cm²) was calculated. After that, a defect review was performed to classify and extract development defects from all defects, and the number of development defects per unit area (items/cm²) was calculated. Values less than 0.5 were rated A, values of 0.5 or more and less than 1.0 were rated B, values of 1.0 or more and less than 5.0 were rated C, and values of 5.0 or more were rated D. The smaller the value, the better the performance.

TABLE 3

| | | | Optimal | | | | | |
|---|---|---|---|---|---|---|---|---|
| | | PEB | exposure | | | | Tilt | |
| | Resist | temperature | amount | EL | LWR | DOF | limit | Development |
| | composition | (° C.) | (mJ/cm²) | (%) | (nm) | (nm) | (nm) | defect |
| Example 3-1 | R-1 | 100 | 32 | 19 | 2.4 | 120 | 10.5 | A |
| Example 3-2 | R-2 | 100 | 33 | 18 | 2.5 | 120 | 10.7 | A |
| Example 3-3 | R-3 | 95 | 34 | 17 | 2.4 | 120 | 10.8 | A |
| Example 3-4 | R-4 | 105 | 32 | 17 | 2.5 | 110 | 10.6 | A |
| Example 3-5 | R-5 | 105 | 34 | 18 | 2.5 | 100 | 10.5 | A |
| Example 3-6 | R-6 | 95 | 32 | 19 | 2.3 | 110 | 11.2 | A |
| Example 3-7 | R-7 | 105 | 33 | 17 | 2.3 | 120 | 11.1 | A |
| Example 3-8 | R-8 | 105 | 32 | 18 | 2.3 | 120 | 11.1 | A |
| Example 3-9 | R-9 | 100 | 33 | 18 | 2.4 | 120 | 10.9 | A |
| Example 3-10 | R-10 | 95 | 35 | 17 | 2.4 | 120 | 10.8 | A |
| Example 3-11 | R-11 | 100 | 32 | 18 | 2.4 | 100 | 10.9 | A |
| Example 3-12 | R-12 | 105 | 32 | 19 | 2.3 | 110 | 11.2 | A |
| Example 3-13 | R-13 | 105 | 33 | 18 | 2.4 | 110 | 10.7 | A |
| Example 3-14 | R-14 | 100 | 34 | 19 | 2.5 | 110 | 11.2 | A |
| Example 3-15 | R-15 | 100 | 33 | 18 | 2.3 | 120 | 10.9 | A |
| Example 3-16 | R-16 | 100 | 33 | 17 | 2.5 | 120 | 10.9 | A |
| Example 3-17 | R-17 | 105 | 32 | 18 | 2.3 | 110 | 11.1 | A |
| Example 3-18 | R-18 | 95 | 34 | 18 | 2.4 | 120 | 10.9 | A |
| Example 3-19 | R-19 | 100 | 32 | 17 | 2.5 | 110 | 10.8 | A |
| Example 3-20 | R-20 | 105 | 33 | 18 | 2.4 | 100 | 11.2 | A |
| Example 3-21 | R-21 | 95 | 34 | 17 | 2.5 | 120 | 11.1 | A |
| Example 3-22 | R-22 | 100 | 33 | 19 | 2.3 | 110 | 10.7 | A |
| Example 3-23 | R-23 | 105 | 33 | 18 | 2.4 | 120 | 11.1 | A |
| Example 3-24 | R-24 | 95 | 33 | 18 | 2.4 | 120 | 11.2 | A |
| Example 3-25 | R-25 | 100 | 32 | 18 | 2.5 | 110 | 10.8 | A |
| Example 3-26 | R-26 | 95 | 33 | 19 | 2.3 | 120 | 10.7 | A |
| Example 3-27 | R-27 | 100 | 34 | 18 | 2.4 | 100 | 10.8 | A |
| Example 3-28 | R-28 | 100 | 33 | 17 | 2.4 | 120 | 11.2 | A |
| Example 3-29 | R-29 | 100 | 32 | 17 | 2.6 | 110 | 11.3 | A |
| Example 3-30 | R-30 | 95 | 34 | 19 | 2.4 | 110 | 11.1 | A |

TABLE 4

| | | | Optimal | | | | | |
|---|---|---|---|---|---|---|---|---|
| | | PEB | exposure | | | | Tilt | |
| | Resist | temperature | amount | EL | LWR | DOF | limit | Development |
| | composition | (° C.) | (mJ/cm²) | (%) | (nm) | (nm) | (nm) | defect |
| Comparative | CR-1 | 100 | 38 | 14 | 3.1 | 80 | 11.7 | B |
| Example 2-1 | | | | | | | | |
| Comparative | CR-2 | 100 | 39 | 14 | 2.7 | 90 | 12.1 | C |
| Example 2-2 | | | | | | | | |
| Comparative | CR-3 | 105 | 40 | 15 | 3.0 | 100 | 12.2 | B |
| Example 2-3 | | | | | | | | |
| Comparative | CR-4 | 100 | 42 | 15 | 3.2 | 80 | 12.2 | B |
| Example 2-4 | | | | | | | | |
| Comparative | CR-5 | 100 | 41 | 14 | 2. | 70 | 12.1 | C |
| Example 2-5 | | | | | | | | |
| Comparative | CR-6 | 100 | 37 | 13 | 3.3 | 80 | 12.1 | C |
| Example 2-6 | | | | | | | | |
| Comparative | CR-7 | 100 | 37 | 15 | 2.8 | 90 | 12.2 | B |
| Example 2-7 | | | | | | | | |
| Comparative | CR-8 | 105 | 38 | 14 | 3.3 | 80 | 12.1 | C |
| Example 2-8 | | | | | | | | |
| Comparative | CR-9 | 100 | 41 | 13 | 2.9 | 100 | 12.3 | C |
| Example 2-9 | | | | | | | | |
| Comparative | CR-10 | 100 | 40 | 13 | 3.0 | 90 | 12.2 | B |
| Example 2-10 | | | | | | | | |
| Comparative | CR-11 | 95 | 41 | 14 | 2.8 | 80 | 12.1 | C |
| Example 2-11 | | | | | | | | |
| Comparative | CR-12 | 100 | 37 | 15 | 2.7 | 80 | 12.1 | B |
| Example 2-12 | | | | | | | | |
| Comparative | CR-13 | 100 | 38 | 16 | 3.1 | 70 | 12.0 | C |
| Example 2-13 | | | | | | | | |
| Comparative | CR-14 | 100 | 38 | 15 | 2.8 | 90 | 11.6 | B |
| Example 2-14 | | | | | | | | |
| Comparative | CR-15 | 100 | 41 | 14 | 2.9 | 80 | 12.2 | C |
| Example 2-15 | | | | | | | | |
| Comparative | CR-16 | 95 | 40 | 15 | 3.0 | 90 | 11.9 | B |
| Example 2-16 | | | | | | | | |
| Comparative | CR-17 | 100 | 37 | 13 | 3.1 | 80 | 12.1 | C |
| Example 2-17 | | | | | | | | |
| Comparative | CR-18 | 105 | 38 | 15 | 2.7 | 80 | 11.8 | B |
| Example 2-18 | | | | | | | | |
| Comparative | CR-19 | 100 | 37 | 14 | 3.1 | 90 | 11.9 | B |
| Example 2-19 | | | | | | | | |
| Comparative | CR-20 | 100 | 39 | 15 | 2.8 | 80 | 12.2 | C |
| Example 2-20 | | | | | | | | |

The results shown in Tables 3 and 4 show that the chemically amplified resist composition containing a photoacid generator made of an onium salt of the present invention has good sensitivity and is excellent in terms of EL, LWR, and DOF. In addition, it was confirmed that the tilt limit value was small, and that the pattern was resistant to tilting even in the formation of fine patterns. Furthermore, it was confirmed that development defects were also suppressed. Therefore, it was shown that the chemically amplified resist composition of the present invention is suitable as a material for EUV lithography.

### [5] EUV Lithography Evaluation (2)

### Examples 4-1 to 4-30, Comparative Examples 3-1 to 3-20

Each chemically amplified resist composition (R-1 to R-30, CR-1 to CR-20) shown in Tables 1 and 2 was spin-coated onto an Si substrate on which a 20-nm-thick silicon-containing spin-on hard mask SHB-A940 (silicon content: 43% by mass) manufactured by Shin-Etsu Chemical Co., Ltd. had been formed, and the substrate was pre-baked at 105° C. for 60 seconds using a hot plate to produce a resist film with a thickness of 50 nm. The resist film was exposed using an EUV scanner NXE:3400 (NA 0.33, σ 0.9/0.6, quadrupole illumination, on-wafer dimension pitch 46 nm, +20% bias hole pattern mask) manufactured by ASML, PEB was performed for 60 seconds using a hot plate at the temperatures listed in Tables 5 and 6, and development was performed for 30 seconds with a 2.38% by mass TMAH aqueous solution to form a hole pattern with a dimension of 23 nm.

Using a CD-SEM (CG6300) manufactured by Hitachi High-Tech Corporation, the exposure amount when a hole dimension of 23 nm was formed was measured, and this was used as the sensitivity; furthermore, the dimensions of 50 holes at this time were also measured, the standard deviation (σ) was calculated, its value was tripled (3σ), and the result was used as the CDU. The results are shown in Tables 5 and 6.

TABLE 5

| | | | Optimal | |
|---|---|---|---|---|
| | | PEB | exposure | |
| | Resist | temperature | amount | CDU |
| | composition | (° C.) | (mJ/cm²) | (nm) |
| Example 4-1 | R-1 | 95 | 22 | 2.2 |
| Example 4-2 | R-2 | 95 | 23 | 2.3 |
| Example 4-3 | R-3 | 95 | 23 | 2.4 |
| Example 4-4 | R-4 | 90 | 22 | 2.3 |
| Example 4-5 | R-5 | 90 | 23 | 2.5 |
| Example 4-6 | R-6 | 95 | 25 | 2.4 |
| Example 4-7 | R-7 | 90 | 24 | 2.5 |
| Example 4-8 | R-8 | 95 | 23 | 2.2 |
| Example 4-9 | R-9 | 90 | 24 | 2.3 |
| Example 4-10 | R-10 | 95 | 23 | 2.4 |
| Example 4-11 | R-11 | 90 | 25 | 2.3 |
| Example 4-12 | R-12 | 90 | 23 | 2.3 |
| Example 4-13 | R-13 | 95 | 24 | 2.3 |
| Example 4-14 | R-14 | 95 | 24 | 2.4 |
| Example 4-15 | R-15 | 95 | 23 | 2.5 |
| Example 4-16 | R-16 | 95 | 23 | 2.4 |
| Example 4-17 | R-17 | 90 | 22 | 2.4 |
| Example 4-18 | R-18 | 90 | 25 | 2.3 |
| Example 4-19 | R-19 | 95 | 25 | 2.3 |
| Example 4-20 | R-20 | 90 | 23 | 2.4 |
| Example 4-21 | R-21 | 95 | 22 | 2.5 |
| Example 4-22 | R-22 | 90 | 23 | 2.4 |
| Example 4-23 | R-23 | 95 | 23 | 2.3 |
| Example 4-24 | R-24 | 90 | 23 | 2.3 |
| Example 4-25 | R-25 | 95 | 22 | 2.5 |
| Example 4-26 | R-26 | 90 | 24 | 2.4 |
| Example 4-27 | R-27 | 95 | 23 | 2.5 |
| Example 4-28 | R-28 | 90 | 25 | 2.3 |
| Example 4-29 | R-29 | 90 | 24 | 2.4 |
| Example 4-30 | R-30 | 95 | 24 | 2.4 |

TABLE 6

| | | | Optimal | |
|---|---|---|---|---|
| | | PEB | exposure | |
| | Resist | temperature | amount | CDU |
| | composition | (° C.) | (mJ/cm²) | (nm) |
| Comparative | CR-1 | 90 | 27 | 2.9 |
| Example 3-1 | | | | |
| Comparative | CR-2 | 90 | 26 | 2.8 |
| Example 3-2 | | | | |
| Comparative | CR-3 | 85 | 27 | 2.7 |
| Example 3-3 | | | | |
| Comparative | CR-4 | 90 | 26 | 2.9 |
| Example 3-4 | | | | |
| Comparative | CR-5 | 95 | 27 | 3.1 |
| Example 3-5 | | | | |
| Comparative | CR-6 | 90 | 28 | 3.0 |
| Example 3-6 | | | | |
| Comparative | CR-7 | 85 | 29 | 2.8 |
| Example 3-7 | | | | |
| Comparative | CR-8 | 90 | 28 | 3.0 |
| Example 3-8 | | | | |
| Comparative | CR-9 | 90 | 27 | 2.9 |
| Example 3-9 | | | | |
| Comparative | CR-10 | 90 | 29 | 2.8 |
| Example 3-10 | | | | |
| Comparative | CR-11 | 85 | 30 | 3.1 |
| Example 3-11 | | | | |
| Comparative | CR-12 | 90 | 29 | 3.2 |
| Example 3-12 | | | | |
| Comparative | CR-13 | 90 | 30 | 3.0 |
| Example 3-13 | | | | |
| Comparative | CR-14 | 90 | 31 | 3.1 |
| Example 3-14 | | | | |
| Comparative | CR-15 | 90 | 27 | 2.8 |
| Example 3-15 | | | | |
| Comparative | CR-16 | 85 | 28 | 2.7 |
| Example 3-16 | | | | |
| Comparative | CR-17 | 95 | 29 | 2.9 |
| Example 3-17 | | | | |
| Comparative | CR-18 | 90 | 27 | 2.8 |
| Example 3-18 | | | | |
| Comparative | CR-19 | 90 | 29 | 2.9 |
| Example 3-19 | | | | |
| Comparative | CR-20 | 90 | 28 | 3.1 |
| Example 3-20 | | | | |

The results shown in Tables 5 and 6 confirm that the chemically amplified resist composition containing a photoacid generator made of an onium salt of the present invention has good sensitivity and is excellent in terms of CDU.

The present description includes the following embodiments.

[1]: An onium salt, wherein the onium salt comprises an anion represented by the following general formula (1A) and a cation represented by the following general formula (1B),

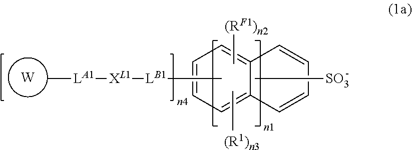

wherein “n1” represents 0 or 1; “n2” represents an integer from 0 to 4; “n3” represents an integer from 0 to 4; however, when “n1” represents 0, 0≤n2+n3≤4, and when “n1” represents 1, 0≤n2+n3≤6; “n4” represents 0 or 1;
W represents a hydrocarbyl group having 6 to 60 carbon atoms and containing at least one aromatic ring, and the hydrocarbyl group may contain a heteroatom;
R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” represents 2, 3, or 4, each R¹ may be the same as or different from each other, and a plurality of R¹ may be bonded to each other to form a ring together with the carbon atoms to which they are bonded;
L^{A1} and L^{B1} each independently represent a single bond, an ether bond, an ester bond, a sulfonate ester bond, an amide bond, a sulfonamide bond, a carbonate bond, or a carbamate bond; and
X^{L1} represents a single bond or a hydrocarbylene group having 1 to 40 carbon atoms and optionally containing a heteroatom;

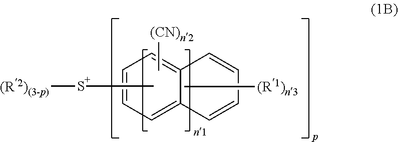

wherein “p” represents 1, 2, or 3; “n′1” represents 0 or 1; “n′2” represents 1 or 2; “n′3” represents an integer from 0 to 6; however, when “n′1” represents 0, 1≤n′2+n′3≤5, and when “n′1” represents 1, 1≤n′2+n′3≤7;
R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n′3” represents 2 to 6, each R′¹ may be the same as or different from each other, and two R′¹ may be bonded to each other to form a ring together with the carbon atoms to which they are bonded;
R′² represents a halogen atom or a hydrocarbyl group having 1 to 30 carbon atoms and optionally containing a heteroatom; when “p” represents 1, two R′² may be the same as or different from each other; and two of the three substituents bonded to S⁺ may be bonded to each other to form a ring together with the sulfur atom to which they are bonded.

[2]: The onium salt according to [1], wherein the W is represented by the following general formulae (W-1) or (W-2),

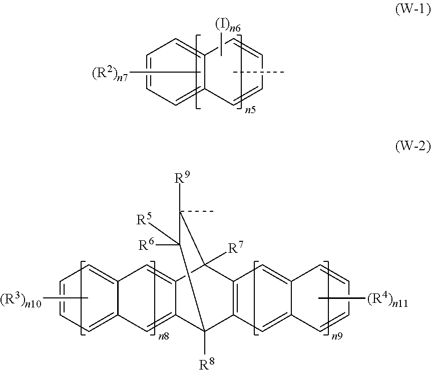

wherein “n5” represents 0 or 1; “n6” represents an integer from 0 to 4; “n7” represents an integer from 1 to 4; however, when “n5” represents 0, 1≤n6+n7≤5, and when “n5” represents 1, 1≤n6+n7≤7; “n8” represents 0 or 1; “n9” represents 0 or 1; “n10” represents an integer from 0 to 4; “n11” represents an integer from 0 to 4;
R² each independently represents a hydrogen atom, a halogen atom other than an iodine atom, or a hydrocarbyl group having 1 to 20 carbon atoms and optionally containing a heteroatom;
R³ and R⁴ each independently represent a hydrogen atom, a halogen atom, or a hydrocarbyl group having 1 to 20 carbon atoms and optionally containing a heteroatom;
R⁵ to R⁹ each independently represent a hydrogen atom, a halogen atom, or a hydrocarbyl group having 1 to 20 carbon atoms and optionally containing a heteroatom; and
a broken line represents an attachment point to L^{A1}.

[3]: The onium salt according to [1] or [2], wherein the anion is represented by the following general formula (1A-1),

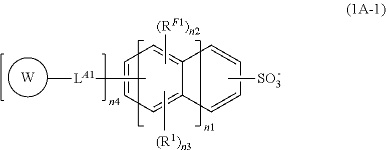

wherein “n1” to “n4”, W, R^{F1}, R¹, and L^{A1} are as defined above.

[4]: The onium salt according to any one of [1] to [3], wherein the cation is represented by the following general formula (1B-1),

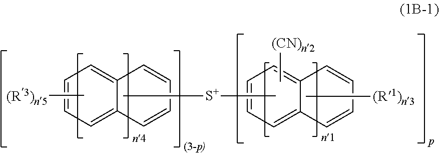

wherein “p”, “n′1” to “n′3”, and R′¹ are as defined above;
“n′4” represents 0 or 1; “n′5” represents an integer from 0 to 5;
R′³ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and when “n′5” represents 2 to 5, each R′³ may be the same as or different from each other, and two R′³ may be bonded to each other to form a ring together with the carbon atoms to which they are bonded.

[5]: A photoacid generator, comprising the onium salt according to any one of [1] to [4].

[6]: A chemically amplified resist composition, comprising the photoacid generator according to [5].

[7]: The chemically amplified resist composition according to [6], further comprising a base polymer containing a repeating unit represented by either one or both of the following general formulas (a1) and (a2),

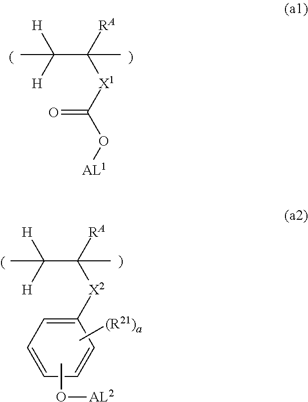

wherein R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
* represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a” represents an integer from 0 to 4.

[8]: The chemically amplified resist composition according to [7], wherein the base polymer further contains a repeating unit represented by the following general formula (a3),

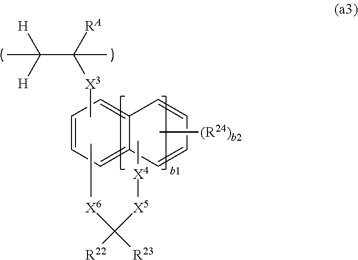

wherein “b1” represents 0 or 1; “b2” represents an integer from 0 to 3 when “b1” is 0, and an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; * represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; however, X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; furthermore, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, a plurality of R²⁴ may be bonded to each other to form a ring together with the carbon atoms of the aromatic ring to which they are bonded.

[9]: The chemically amplified resist composition according to [7] or [8], wherein the base polymer further contains a repeating unit represented by either one or both of the following general formulas (b1) and (b2),

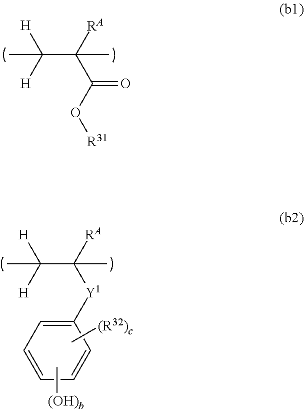

wherein R^{A} each independently represents a hydrogen atom, a fluorine atom, a methyl group, or a trifluoromethyl group;
Y¹ represents a single bond or *—C(═O)—O—; * represents an attachment point to a main chain carbon atom;
R³¹ represents a hydrogen atom, or a group having 1 to 20 carbon atoms and containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic acid anhydride (—C(═O)—O—C(═O)—);
R³² represents a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “c” is 2 or more, each R³² may be the same as or different from each other;
“b” represents an integer from 1 to 4; and “c” represents an integer from 0 to 4; however, 1≤b+c≤5.
[10]: The chemically amplified resist composition according to any one of [7] to [9], wherein the base polymer further comprises at least one repeating unit selected from the group consisting of a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5),

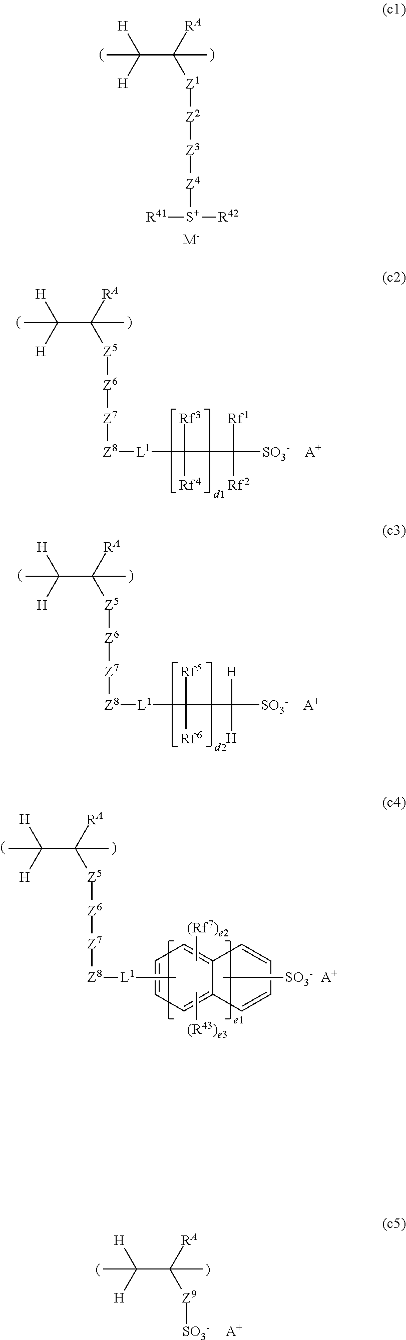

wherein “d1” and “d2” each independently represent an integer from 0 to 3;
“e1” represents 0 or 1; “e2” represents an integer from 0 to 4; “e3” represents an integer from 0 to 4; however, when “e1” represents 0, 0≤e2+e3≤4, and when “e1” represents 1, 0≤e2+e3≤6;
R^{A} each independently represents a hydrogen atom, a fluorine atom, a methyl group, or a trifluoromethyl group;
Z¹ represents a single bond or a phenylene group which may have a substituent;
Z² represents a single bond, **—C(═O)—O—Z²¹—, **—C(═O)—NH—Z²¹—, or **—O—Z²¹—; Z²¹ represents an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ represents a single bond, an ether bond, an ester bond, a sulfonate ester bond, an amide bond, a sulfonamide bond, a carbonate bond, or a carbamate bond;
Z⁴ represents a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ each independently represents a single bond, a phenylene group or naphthylene group which may have a substituent, or *—C(═O)—O—Z⁵¹—; Z⁵¹ represents an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ represents a single bond, an ether bond, an ester bond, a sulfonate ester bond, an amide bond, a sulfonamide bond, a carbonate bond, or a carbamate bond;
Z⁷ each independently represents a single bond, ***—Z⁷¹—C(O)—O—, ***—C(═O)—NH—Z⁷¹—, or ***—O—Z⁷¹; Z⁷?represents a hydrocarbylene group having 1 to 20 carbon atoms and optionally containing a heteroatom;
Z⁸ each independently represents a single bond, ****—Z⁸¹—C(═O)—O—, ****—C(═O)—NH—Z⁸¹—, or ****—O—Z⁸¹—; Z⁸¹ represents a hydrocarbylene group having 1 to 20 carbon atoms and optionally containing a heteroatom;
Z⁹ represents a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—; Z⁹¹ represents an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
* represents an attachment point to a main chain carbon atom; “**” represents an attachment point to Z¹; “***” represents an attachment point to Z⁶; “****” represents an attachment point to Z⁷;
L¹ represents a single bond, an ether bond, an ester bond, a carbonyl group, a sulfonate ester bond, a sulfonamide bond, a carbonate bond, or a carbamate bond; Rf¹ and Rf² each independently represent a fluorine atom or a fluorinated saturated hydrocarbyl group having 1 to 6 carbon atoms;
Rf³ and Rf⁴ each independently represent a hydrogen atom, a fluorine atom, or a fluorinated saturated hydrocarbyl group having 1 to 6 carbon atoms;
Rf⁵ and Rf⁶ each independently represent a hydrogen atom, a fluorine atom, or a fluorinated saturated hydrocarbyl group having 1 to 6 carbon atoms; however, all Rf⁵ and Rf⁶ cannot simultaneously become a hydrogen atom;
Rf⁷ represents a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² each independently represent a hydrocarbyl group having 1 to 20 carbon atoms and optionally containing a heteroatom; furthermore, R⁴¹ and R⁴² may be bonded to each other to form a ring together with the sulfur atom to which they are bonded;
R⁴³ represents a halogen atom other than a fluorine atom, or a hydrocarbyl group having 1 to 20 carbon atoms and optionally containing a heteroatom; when “e3” is 2, 3, or 4, a plurality of R⁴³ may be bonded to each other to form a ring together with the carbon atoms to which they are bonded;
M⁻ represents a non-nucleophilic counterion; and
A⁺ represents an onium cation.

[11]: The chemically amplified resist composition according to any one of [6] to [10], further comprising at least one selected from the group consisting of an organic solvent, a quencher, a photoacid generator other than the photoacid generator, and a surfactant.

[12]: A patterning process, comprising the steps of: forming a resist film on a substrate using the chemically amplified resist composition according to any one of [6] to [11]; exposing the resist film with a high-energy beam; and developing the exposed resist film using a developer.

[13]: The patterning process according to [12], wherein the high-energy beam is KrF excimer laser light, ArF excimer laser light, an electron beam, or extreme ultraviolet having a wavelength of 3 to 15 nm.

It should be noted that the present invention is not limited to the above-described embodiments. The embodiments are just examples, and any examples that have substantially the same features and demonstrate the same functions and effects as those in the technical concepts disclosed in the claims of the present invention are included in the technical scope of the present invention.

1. An onium salt, wherein the onium salt comprises an anion represented by the following general formula (1A) and a cation represented by the following general formula (1B), 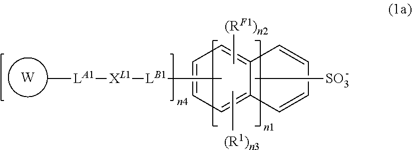
wherein “n1” represents 0 or 1; “n2” represents an integer from 0 to 4; “n3” represents an integer from 0 to 4; however, when “n1” represents 0, 0≤n2+n3≤4, and when “n1” represents 1, 0≤n2+n3≤6; “n4” represents 0 or 1;
W represents a hydrocarbyl group having 6 to 60 carbon atoms and containing at least one aromatic ring, and the hydrocarbyl group may contain a heteroatom;
R^{F1} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ represents a halogen atom other than fluorine, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” represents 2, 3, or 4, each R¹ may be the same as or different from each other, and a plurality of R¹ may be bonded to each other to form a ring together with the carbon atoms to which they are bonded;
L^{A1} and L^{B1} each independently represent a single bond, an ether bond, an ester bond, a sulfonate ester bond, an amide bond, a sulfonamide bond, a carbonate bond, or a carbamate bond; and
X^{L1} represents a single bond or a hydrocarbylene group having 1 to 40 carbon atoms and optionally containing a heteroatom; 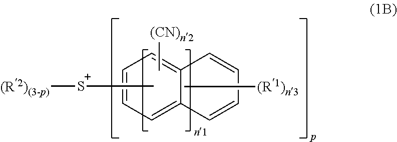
wherein “p” represents 1, 2, or 3; “n′1” represents 0 or 1; “n′2” represents 1 or 2; “n′3” represents an integer from 0 to 6; however, when “n′1” represents 0, 1≤n′2+n′3≤5, and when “n′1” represents 1, 1≤n′2+n′3≤7;
R′¹ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n′3” represents 2 to 6, each R′¹ may be the same as or different from each other, and two R′¹ may be bonded to each other to form a ring together with the carbon atoms to which they are bonded;
R′² represents a halogen atom or a hydrocarbyl group having 1 to 30 carbon atoms and optionally containing a heteroatom; when “p” represents 1, two R′² may be the same as or different from each other; and two of the three substituents bonded to S⁺ may be bonded to each other to form a ring together with the sulfur atom to which they are bonded.

2. The onium salt according to claim 1, wherein the W is represented by the following general formulae (W-1) or (W-2), 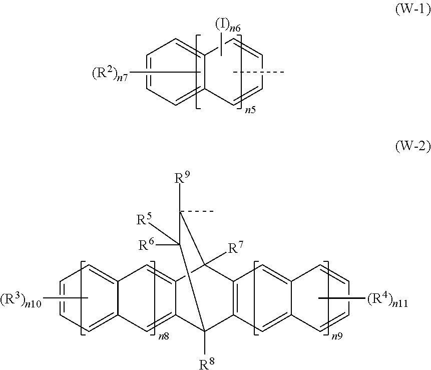
wherein “n5” represents 0 or 1; “n6” represents an integer from 0 to 4; “n7” represents an integer from 1 to 4; however, when “n5” represents 0, 1≤n6+n7≤5, and when “n5” represents 1, 1≤n6+n7≤7; “n8” represents 0 or 1; “n9” represents 0 or 1; “n10” represents an integer from 0 to 4; “n11” represents an integer from 0 to 4;
R² each independently represents a hydrogen atom, a halogen atom other than an iodine atom, or a hydrocarbyl group having 1 to 20 carbon atoms and optionally containing a heteroatom;
R³ and R⁴ each independently represent a hydrogen atom, a halogen atom, or a hydrocarbyl group having 1 to 20 carbon atoms and optionally containing a heteroatom;
R⁵ to R⁹ each independently represent a hydrogen atom, a halogen atom, or a hydrocarbyl group having 1 to 20 carbon atoms and optionally containing a heteroatom; and
a broken line represents an attachment point to L^{A1}.

3. The onium salt according to claim 1, wherein the anion is represented by the following general formula (1A-1), 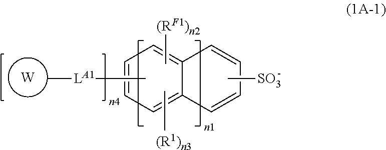
wherein “n1” to “n4”, W, R^{F1}, R¹, and L^{A1} are as defined above.

4. The onium salt according to claim 1, wherein the cation is represented by the following general formula (1B-1), 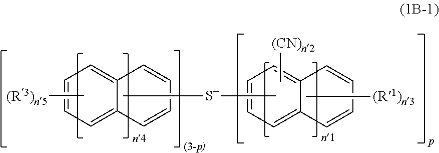
wherein “p”, “n′1” to “n′3”, and R′¹ are as defined above;
“n′4” represents 0 or 1; “n′5” represents an integer from 0 to 5;
R′³ represents a halogen atom, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and when “n′5” represents 2 to 5, each R′³ may be the same as or different from each other, and two R′³ may be bonded to each other to form a ring together with the carbon atoms to which they are bonded.

5. A photoacid generator, comprising the onium salt according to claim 1.

6. A chemically amplified resist composition, comprising the photoacid generator according to claim 5.

7. The chemically amplified resist composition according to claim 6, further comprising a base polymer containing a repeating unit represented by either one or both of the following general formulas (a1) and (a2), 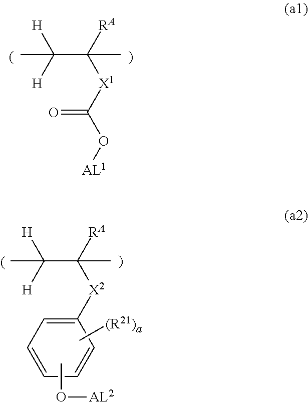
wherein R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
* represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a” represents an integer from 0 to 4.

8. The chemically amplified resist composition according to claim 7, wherein the base polymer further contains a repeating unit represented by the following general formula (a3), 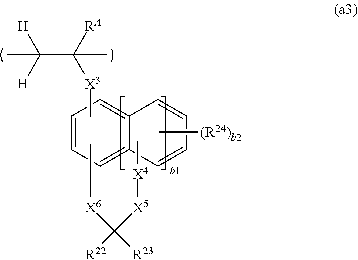
wherein “b1” represents 0 or 1; “b2” represents an integer from 0 to 3 when “b1” is 0, and an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; * represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; however, X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; furthermore, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, a plurality of R²⁴ may be bonded to each other to form a ring together with the carbon atoms of the aromatic ring to which they are bonded.

9. The chemically amplified resist composition according to claim 7, wherein the base polymer further contains a repeating unit represented by either one or both of the following general formulas (b1) and (b2), 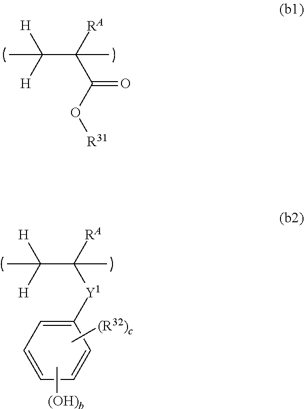
wherein R^{A} each independently represents a hydrogen atom, a fluorine atom, a methyl group, or a trifluoromethyl group;
Y¹ represents a single bond or *—C(═O)—O—; * represents an attachment point to a main chain carbon atom;
R³¹ represents a hydrogen atom, or a group having 1 to 20 carbon atoms and containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic acid anhydride (—C(═O)—O—C(═O)—);
R³² represents a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “c” is 2 or more, each R³² may be the same as or different from each other;
“b” represents an integer from 1 to 4; and “c” represents an integer from 0 to 4; however, 1≤b+c≤5.

10. The chemically amplified resist composition according to claim 7, wherein the base polymer further comprises at least one repeating unit selected from the group consisting of a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5), 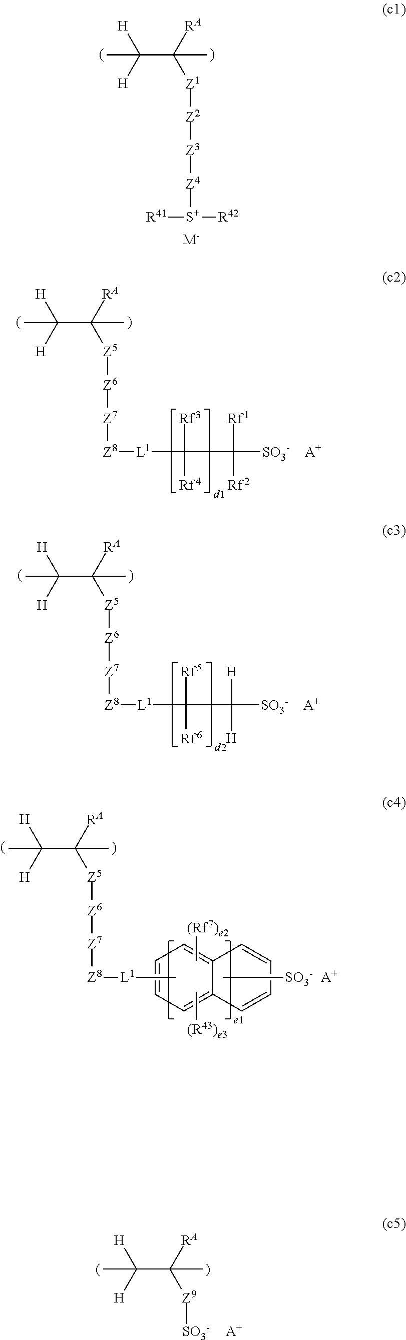
wherein “d1” and “d2” each independently represent an integer from 0 to 3;
“e1” represents 0 or 1; “e2” represents an integer from 0 to 4; “e3” represents an integer from 0 to 4;
however, when “e1” represents 0, 0≤e2+e3≤4, and when “e1” represents 1, 0≤e2+e3≤6;
R^{A} each independently represents a hydrogen atom, a fluorine atom, a methyl group, or a trifluoromethyl group;
Z¹ represents a single bond or a phenylene group which may have a substituent;
Z² represents a single bond, **—C(═O)—O—Z²¹—, **—C(═O)—NH—Z²¹—, or **—O—Z²¹—; Z²¹ represents an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ represents a single bond, an ether bond, an ester bond, a sulfonate ester bond, an amide bond, a sulfonamide bond, a carbonate bond, or a carbamate bond;
Z⁴ represents a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ each independently represents a single bond, a phenylene group or naphthylene group which may have a substituent, or *—C(═O)—O—Z⁵¹—; Z⁵¹ represents an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ represents a single bond, an ether bond, an ester bond, a sulfonate ester bond, an amide bond, a sulfonamide bond, a carbonate bond, or a carbamate bond;
Z⁷ each independently represents a single bond, ***—Z⁷¹—C(═O)—O—, ***—C(═O)—NH—Z⁷¹—, or ***—O—Z⁷¹—; Z⁷ represents a hydrocarbylene group having 1 to 20 carbon atoms and optionally containing a heteroatom;
Z⁸ each independently represents a single bond, ****—Z⁸¹—C(═O)—O—, ****—C(═O)—NH—Z⁸¹—, or ****—O—Z⁸¹—; Z⁸¹ represents a hydrocarbylene group having 1 to 20 carbon atoms and optionally containing a heteroatom;
Z⁹ represents a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—; Z⁹¹ represents an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
* represents an attachment point to a main chain carbon atom; “**” represents an attachment point to Z¹; “***” represents an attachment point to Z⁶; “****” represents an attachment point to Z⁷;
L¹ represents a single bond, an ether bond, an ester bond, a carbonyl group, a sulfonate ester bond, a sulfonamide bond, a carbonate bond, or a carbamate bond;
Rf¹ and Rf² each independently represent a fluorine atom or a fluorinated saturated hydrocarbyl group having 1 to 6 carbon atoms;
Rf³ and Rf⁴ each independently represent a hydrogen atom, a fluorine atom, or a fluorinated saturated hydrocarbyl group having 1 to 6 carbon atoms;
Rf⁵ and Rf⁶ each independently represent a hydrogen atom, a fluorine atom, or a fluorinated saturated hydrocarbyl group having 1 to 6 carbon atoms; however, all Rf⁵ and Rf⁶ cannot simultaneously become a hydrogen atom;
Rf⁷ represents a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² each independently represent a hydrocarbyl group having 1 to 20 carbon atoms and optionally containing a heteroatom; furthermore, R⁴¹ and R⁴² may be bonded to each other to form a ring together with the sulfur atom to which they are bonded;
R⁴³ represents a halogen atom other than a fluorine atom, or a hydrocarbyl group having 1 to 20 carbon atoms and optionally containing a heteroatom; when “e3” is 2, 3, or 4, a plurality of R⁴³ may be bonded to each other to form a ring together with the carbon atoms to which they are bonded;
M⁻ represents a non-nucleophilic counterion; and
A⁺ represents an onium cation.

11. The chemically amplified resist composition according to claim 6, further comprising at least one selected from the group consisting of an organic solvent, a quencher, a photoacid generator other than the photoacid generator, and a surfactant.

12. A patterning process, comprising the steps of: forming a resist film on a substrate using the chemically amplified resist composition according to claim 6; exposing the resist film with a high-energy beam; and developing the exposed resist film using a developer.

13. The patterning process according to claim 12, wherein the high-energy beam is KrF excimer laser light, ArF excimer laser light, an electron beam, or extreme ultraviolet having a wavelength of 3 to 15 nm.
